(19) United States
(12) Patent Application Publication
(10) Pub. No.: US 2024/0182464 A1
(43) Pub. Date: Jun. 6, 2024

(54) SELECTIVE BCL-XL PROTAC COMPOUNDS AND METHODS OF USE

(71) Applicants: Les Laboratoirens Serviece, Suresnes Cedex (FR); Novartis AG, Basel (CH)

(72) Inventors: András Herner, Tata (HU); Piroska Markacz, Budapest (HU); Gang Liu, Cambridge, MA (US); Katsumasa Nakajima, Cambridge, MA (US); Tibor Novak, Budapest (HU); Georges-Alain Franzetti, Paris (FR); Jérôme-Benoît Starck, Rueil-Malmaison (FR); Frédéric Colland, Puisux-en-France (FR); Patrice Desos, Suresnes (FR); Attila Paczal, Budapest (HU); Bianka Szokol, Dorog (HU); Mátyás Pál Timari, Budapest (HU); Vilibald Kun, Tárnok (HU); Matthew T. Burger, Cambridge, MA (US); Joseph Anthony D'Alessio, Cambridge, MA (US); Katherine Elizabeth Seiss, Cambridge, MA (US); Rany Mathew Thomas, Cambridge, MA (US); Liang Zhao, Cambridge, MA (US)

(21) Appl. No.: 18/275,379

(22) PCT Filed: Feb. 1, 2022
(86) PCT No.: PCT/US2022/014790

§ 371 Date: Aug. 1, 2023

Related U.S. Application Data
(60) Provisional application No. 63/144,577, filed on Feb. 2, 2021.

(51) Int. Cl. C07D 471/04; A61P 35/00; C07D 417/14

(52) U.S. Cl. CPC C07D 471/04; A61P 35/00; C07D 417/14

(57) ABSTRACT

The present disclosure provides PROTAC compounds represented by Formula (A): D-L-DSM (A), or an enantiomer, a diastereoisomer, and/or a pharmaceutically acceptable salt of any one of the foregoing, wherein: DSM is a degradation signaling compound {e.g., an E3 ubiquitin ligase recruitment ligand, such as a CRBN ligand or a VHL ligand) covalently attached to a linker L; L is a linker that covalently attaches DSM to D; and D is a Bcl-xL inhibitor compound of Formula (I) or Formula (II) covalently attached to the linker L: (I); (II) wherein the definitions for the variables are described herein. Also provided are pharmaceutical compositions comprising the PROTAC compounds of the present disclosure and methods of use and methods of making thereof.

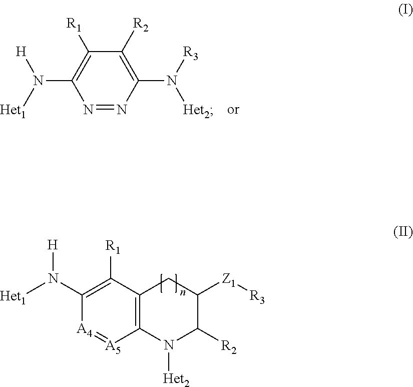

### RELATED APPLICATION

This application claims the benefit of and priority to the filing date under 35 U.S.C. § 119(e) of U.S. Provisional Application No. 63/144,577, filed on Feb. 2, 2021, the entire content of which is incorporated herein by reference in its entirety.

### FIELD OF THE INVENTION

The present disclosure relates to compounds comprising a Bcl-xL inhibiting moiety covalently linked to a degradation signaling moiety (DSM) that binds to a degradation protein or degradation protein complex, e.g., an E3 ubiquitin ligase or an E3 ubiquitin ligase complex. The disclosure further relates to methods and compositions useful in the treatment of cancers that are responsive to decrease in Bcl-xL expression and/or activity.

### BACKGROUND OF THE INVENTION

Apoptosis (programmed cell death) is an evolutionarily conserved pathway essential for tissue homeostasis, development and removal of damaged cells. Deregulation of apoptosis contributes to human diseases, including malignancies, neurodegenerative disorders, diseases of the immune system and autoimmune diseases (Hanahan and Weinberg, Cell. 2011 Mar. 4; 144(5):646-74; Marsden and Strasser, Annu Rev Immunol. 2003; 21:71-105; Vaux and Flavell, Curr Opin Immunol. 2000 December; 12(6):719-24). Evasion of apoptosis is recognized as a hallmark of cancer, participating in the development as well as the sustained expansion of tumors and the resistance to anti-cancer treatments (Hanahan and Weinberg, Cell. 2000 Jan. 7; 100(1):57-70).

The Bcl-2 protein family comprises key regulators of cell survival which can suppress (e.g., Bcl-2, Bcl-xL, Mcl-1) or promote (e.g., Bad, Bax) apoptosis (Gross et al., Genes Dev. 1999 Aug. 1; 13(15):1899-911, Youle and Strasser, Nat. Rev. Mol. Cell Biol. 2008 January; 9(1):47-59). In the face of stress stimuli, whether a cell survives or undergoes apoptosis is dependent on the extent of pairing between the Bcl-2 family members that promote cell death with family members that promote cell survival. For the most part, these interactions involve the docking of the Bcl-2 homology 3 (BH3) domain of proapoptotic family members into a groove on the surface of pro-survival members. The presence of Bcl-2 homology (BH) domain defines the membership of the Bcl-2 family, which is divided into three main groups depending upon the particular BH domains present within the protein. The prosurvival members such as Bcl-2, Bcl-xL, and Mcl-1 contain BH domains 1-4, whereas Bax and Bak, the proapoptotic effectors of mitochondrial outer membrane permeabilization during apoptosis, contain BH domains 1-3 (Youle and Strasser, Nat. Rev. Mol. Cell Biol. 2008 January; 9(1):47-59).

Overexpression of the prosurvival members of the Bcl-2 family is a hallmark of cancer and it has been shown that these proteins play an important role in tumor development, maintenance and resistance to anticancer therapy (Czabotar et al., Nat. Rev. Mol. Cell Biol. 2014 January; 15(1):49-63). Bcl-xL (also named BCL2L1, from BCL2-like 1) is frequently amplified in cancer (Beroukhim et al., Nature 2010 Feb. 18; 463(7283):899-905) and it has been shown that its expression inversely correlates with sensitivity to more than 120 anti-cancer therapeutic molecules in a representative panel of cancer cell lines (NCI-60) (Amundson et al., Cancer Res. 2000 Nov. 1; 60(21):6101-10).

In addition, several studies using transgenic knockout mouse models and transgenic overexpression of Bcl-2 family members highlighted the importance of these proteins in the diseases of the immune system and autoimmune diseases (for a review, see Merino et al., Apoptosis 2009 April; 14(4):570-83. doi: 10.1007/s10495-008-0308-4.PMID: 19172396). Transgenic overexpression of Bcl-xL within the T-cell compartment resulted in resistance to apoptosis induced by glucocorticoid, g-radiation and CD3 crosslinking, suggesting that transgenic Bcl-xL overexpression can reduce apoptosis in resting and activated T-cells (Droin et al., Biochim Biophys Acta 2004 Mar. 1; 1644(2-3):179-88. doi: 10.1016/j.bbamcr.2003.10.011. PMID: 14996502).

In patient samples, persistent or high expression of anti-apoptotic Bcl-2 family proteins has been observed (Pope et al., Nat Rev Immunol. 2002 July; 2(7):527-35. doi: 10.1038/nri846.PMID: 12094227). In particular, T-cells isolated from the joints of rheumatoid arthritis patients exhibited increased Bcl-xL expression and were resistant to spontaneous apoptosis (Salmon et al., J Clin Invest. 1997 Feb. 1; 99(3):439-46. doi: 10.1172/JCI119178.PMID: 9022077). The use of BH3 mimetics has also shown benefit in pre-clinical models of diseases of the immune system and autoimmune diseases. Treatment with ABT-737 (Bcl-2, Bcl-xL, and Bcl-w inhibitor) resulted in potent inhibition of lymphocyte proliferation in vitro. Importantly, mice treated with ABT-737 in animal models of arthritis and lupus showed a significant decrease in disease severity (Bardwell et al., J Clin Invest. 1997 Feb. 1; 99(3):439-46. doi: 10.1172/JCI119178.PMID: 9022077). In addition, it has been shown that ABT-737 prevented allogeneic T-cell activation, proliferation, and cytotoxicity in vitro and inhibited allogeneic T- and B-cell responses after skin transplantation with high selectivity for lymphoid cells (Cippa et al., Transpl Int. 2011 July; 24(7):722-32. doi: 10.1111/j.1432-2277.2011.01272.x. Epub 2011 May 25.PMID: 21615547).

The findings indicated above motivated the discovery and development of a new class of drugs named BH3 mimetics. These molecules are able to disrupt the interaction between the proapoptotic and anti-apoptotic members of the Bcl-2 family and are potent inducers of apoptosis. This new class of drugs includes inhibitors of Bcl-2, Bcl-xL, Bcl-w and Mcl-1. The first BH3 mimetics described were ABT-737 and ABT-263, targeting Bcl-2, Bcl-xL and Bcl-w (Park et al., J. Med. Chem. 2008 Nov. 13; 51(21):6902-15; Roberts et al., J. Clin. Oncol. 2012 Feb. 10; 30(5):488-96). After that, selective inhibitors of Bcl-2 (ABT-199 and S55746—Souers et al., Nat Med. 2013 February; 19(2):202-8; Casara et al., Oncotarget 2018 Apr. 13; 9(28):20075-20088), Bcl-xL (A-1155463 and A-1331852—Tao et al., ACS Med Chem Lett. 2014 Aug. 26; 5(10):1088-93; Leverson et al., Sci Transl Med. 2015 Mar. 18; 7(279):279ra40) and Mcl-1 (A-1210477, S63845, S64315, AMG-176 and AZD-5991—Leverson et al., Cell Death Dis. 2015 Jan. 15; 6:e1590.; Kotschy et al., Nature 2016, 538, 477-482; Maragno et al., AACR2019, Poster #4482; Kotschy et al., WO 2015/097123; Caenepeel et al., Cancer Discov. 2018 December; 8(12):1582-1597; Tron et al., Nat. Commun. 2018 Dec. 17; 9(1):5341) were also discovered. The selective Bcl-2 inhibitor ABT-199 is now approved for the treatment of patients with CLL and AML in combination therapy, while the other inhibitors are still under pre-clinical or clinical development. In pre-clinical models, ABT-263 has shown activity in several hematological malignancies and solid tumors (Shoemaker et al., Clin. Cancer Res. 2008 Jun. 1; 14(11):3268-77; Ackler et al., Cancer Chemother. Pharmacol. 2010 October; 66(5):869-80; Chen et al., Mol. Cancer Ther. 2011 December; 10(12):2340-9). In clinical studies, ABT-263 exhibited objective antitumor activity in lymphoid malignancies (Wilson et al., Lancet Oncol. 2010 December; 11(12):1149-59; Roberts et al., J. Clin. Oncol. 2012 Feb. 10; 30(5):488-96) and its activity is being investigated in combination with several therapies in solid tumors. The selective Bcl-xL inhibitors, A-1155463 or A-1331852, exhibited in vivo activity in pre-clinical models of T-ALL (T-cell Acute Lymphoblastic Leukemia) and different types of solid tumors (Tao et al., ACS Med. Chem. Lett. 2014 Aug. 26; 5(10):1088-93; Leverson et al., Sci. Transl. Med. 2015 Mar. 18; 7(279):279ra40). The Mcl-1 selective inhibitors have shown promising in vivo activity in several types of hematological cell malignancies in preclinical models and three of them, S64315, AMG176 and AZD5991, are currently being investigated in clinical trials (Yang et al., Eur. J. Med. Chem. 2019 May 8; 177:63-75). Therefore, BH3 mimetics represent a highly attractive approach for the development of novel therapies in oncology and in the field of immune and autoimmune diseases.

To further the development of alternative BH3 mimetics, a new approach could involve their use as both protein binders and inhibitors. Indeed BH3 mimetics, and more specifically Bcl-xL inhibitors due to the medical need, can be upgraded by converting their mechanism of action from inhibition to degradation via PROTAC (Proteolysis-targeting chimeras). These degraders are small molecules including (i) a ligand targeting a protein of interest (BH3) to be degraded, (ii) an E3 ubiquitin ligase recruitment ligand (mainly Cereblon (CRBN) or Von Hippel-Lindau (VHL) ligands), and (iii) a chemical linker connecting the two ligands. Upon PROTAC-mediated heterodimerization of the two bound proteins, the target protein is ubiquitinated and degraded by the proteasome (Sakamoto et al., PNAS 2001 17; 98(15):8554-8559; Schneekloth et al., J. Am. Chem. Soc. 2004 31; 126(12): 3748-3754.).

This method has several advantages. First, potency could be improved by using the potential benefit from the catalytic process of the PROTAC approach compared to a stoichiometric inhibition (Bondeson et al., Cell Chem. Biol. 2018 Jan. 18; 25(1):78-87; Qin et al., J. Med. Chem. 2018 61(15) 6685-6704). Moreover, selectivity can be achieved due to the site-specific expression of E3 ligases (Schapira et al., Nat. Rev. Drug Discov. 2019 December; 18(12):949-963). Finally, the first PROTAC ARV-110 entered in clinical trials in Q1-2019. This orally available compound is designed to degrade the androgen receptor (AR) in prostate cancer. Another compound (ARV-471) designed to degrade the Estrogen receptor also entered in clinical trials in Q3-2019.

Several BH3 mimetics have been converted into PROTACs to date, such as Bcl-xL/Bcl-2 dual inhibitors (DT2216 and XZ-739 based on ABT-263 scaffold, Khan et al., Nat Med 2019 December; 25(12):1938-1947; Zhang et al., Eur. J. of Med. Chem. 2020 Apr. 15; 192:112186), Bcl-xL inhibitors (XZ-424 based on A-1155463 scaffold, Zhang et al., Chem. Commun. 2019 Dec. 5; 55(98):14765-14768), Mcl-1 inhibitors (dMCL1-2 based on A-1210477 scaffold, Papatzimas et al., J. Med. Chem. 2019 62(11):5522-5540), and Bcl-2/Mcl-1 dual inhibitors (Wang et al., J. Med. Chem. 2019 Sep. 12; 62(17):8152-8163). Interestingly, because VHL and CRBN E3 ligases are minimally expressed in platelets, a Bcl-xL PROTAC based on ABT-263 can selectively induce Bcl-xL degradation in various cancer cells but not in platelets; thus, potentially sparing dose-limiting platelet toxicity.

Therefore, BH3 mimetics-based degraders represent a highly attractive approach for developing novel therapies in oncology and in the field of immune and autoimmune diseases. In particular, a need exists for potent PROTACs that selectively degrade the Bcl-xL protein. As described herein, the present disclosure fulfills this need.

### SUMMARY OF THE INVENTION

The present disclosure provides, in part, potent and selective Bcl-xL PROTAC degraders of formula (A), as defined below. Based on their pro-apoptotic properties, these compounds could be of interest for treatment of pathologies involving a deregulation of apoptosis such as, for example, cancer, autoimmune diseases and disease of the immune system. These compounds may slow, inhibit, and/or reverse tumor growth in mammals, and/or may be useful for treating human cancer patients. The present disclosure more specifically relates, in some embodiments, to pro-apoptotic agents that are capable of binding and killing cancer cells. In some embodiments, the pro-apoptotic agents are PROTAC compounds comprising a linker that attaches a Bcl-xL inhibitor to an E3 ubiquitin ligase recruitment ligand.

In a first embodiment of the present disclosure, PROTAC compounds of the present disclosure can be represented by Formula (A):

D-L-DSM (A),

or an enantiomer, a diastereoisomer, and/or a pharmaceutically acceptable salt of any one of the foregoing, wherein:
DSM is a degradation signaling compound covalently attached to the linker L;
L is a linker that covalently attaches DSM to D; and
D is a Bcl-xL inhibitor compound of Formula (I) or Formula (II) covalently attached to the linker L:

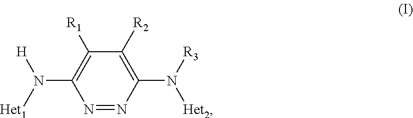

or an enantiomer, a diastereoisomer, and/or a pharmaceutically acceptable salt of any one of the foregoing, wherein:
R₁ and R₂ independently of one another represent a group selected from the group consisting of: hydrogen; a linear or branched C₁-C₆alkyl optionally substituted by a hydroxyl or a C₁-C₆alkoxy group; a C₃-C₆cycloalkyl; a trifluoromethyl; and a linear or branched C₁-C₆alkylene-heterocycloalkyl wherein the heterocycloalkyl group is optionally substituted by a linear or branched C₁-C₆alkyl group;
   or R₁ and R₂ form with the carbon atoms carrying them a C₃-C₆cycloalkylene group,
R₃ represents a group selected from the group consisting of: hydrogen; a C₃-C₆cycloalkyl; a linear or branched C₁-C₆alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:
—X₁-≡CH,
Rₐ and R_{b} independently of one another represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl groups; a C₁-C₆alkylene-SO₂OH; a C₁-C₆alkylene-SO₂O—; a C₁-C₆alkylene-COOH; a C₁-C₆alkylene-PO(OH)₂; a C₁-C₆alkylene-NR_{d}Rₑ; a C₁-C₆alkylene-N⁺R_{d}RₑR_{f}; a C₁-C₆alkylene-phenyl wherein the phenyl may be substituted by a C₁-C₆alkoxy group; and the group:

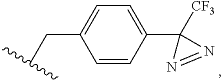 [SMILES: *Cc1ccc(C2(C(F)(F)F)N=N2)cc1]

or Rₐ and R_{b} form with the nitrogen atom carrying them a cycle B₁;
or Rₐ, R_{b} and R_{c} form with the nitrogen atom carrying them a bridged C₃-C₅hetero cycloalkyl,
R_{c}, R_{d}, Rₑ, R_{f}, independently of one another represents a hydrogen or a linear or branched C₁-C₆alkyl group,
   or R_{d} and Rₑ form with the nitrogen atom carrying them a cycle B₂,
   or R_{d}, Rₑ and R_{f} form with the nitrogen atom carrying them a bridged C₃-C₅hetero cycloalkyl,
Het₁ represents a group selected from the group consisting of:

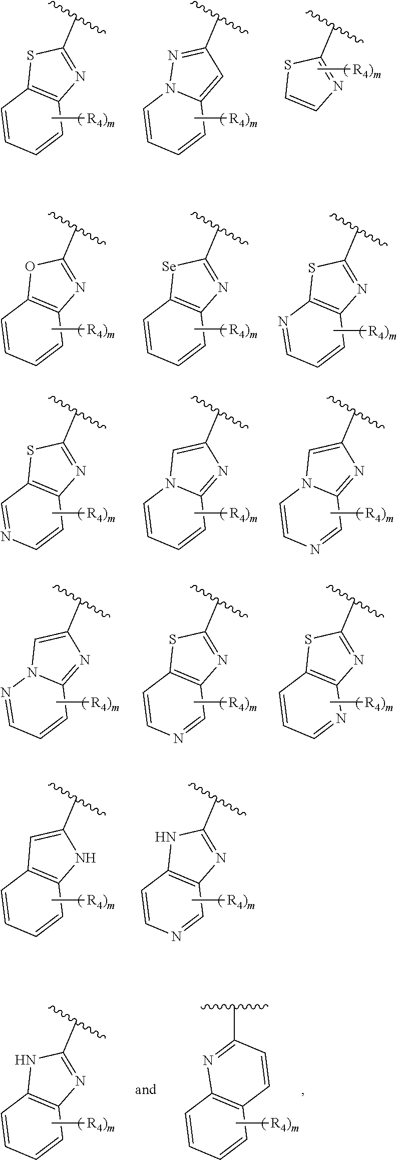

Het₂ represents a group selected from the group consisting of:

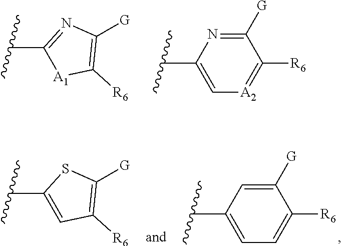

A₁ is —NH—, —N(C₁-C₃alkyl), O, S or Se,
A₂ is N, CH or C(R₅),
G is selected from the group consisting of: —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, —C₁-C₆alkyl optionally substituted by a hydroxyl group, a halogen, —NO₂, and —CN, in which:
R_{G1} and R_{G2} at each occurrence are each independently selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂-C₆alkynyl, a C₃-C₆cycloalkyl, phenyl and —(CH₂)₁₋₄-phenyl;
R_{G3} is selected from the group consisting of a C₁-C₆alkyl optionally substituted by 1 to 3 halogen atoms, a C₂-C₆alkenyl, a C₂-C₆alkynyl, a C₃-C₆cycloalkyl, phenyl and —(CH₂)₁₋₄-phenyl; or R_{G1} and R_{G2}, together with the atom to which each is attached are combined to form a C₃-C₈heterocycloalkyl; or in the alternative, G is selected from the group consisting of:

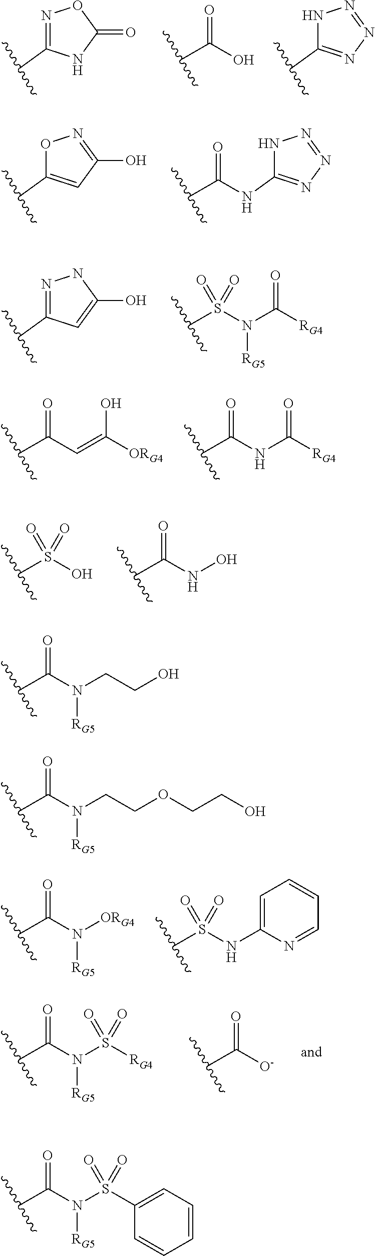

wherein R_{G4} is selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂-C₆alkynyl and a C₃-C₆cycloalkyl,
   and R_{G5} represents a hydrogen atom or a C₁-C₆alkyl group optionally substituted by 1 to 3 halogen atoms,
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a C₂-C₆alkenyl; a C₂-C₆alkynyl; a halogen; and —CN,
R₆ represents a group selected from the group consisting of: hydrogen;
a linear or branched —C₁-C₆alkylene-R₈ group;
a —C₂-C₆alkenyl;
—X₂—O—R⁷;

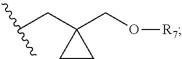

—X₂—NSO₂—R₇;
—C═C(R₉)—Y₁—O—R⁷;
a C₃-C₆cycloalkyl;
a C₃-C₆heterocycloalkyl optionally substituted by a hydroxyl group;
a C₃-C₆cycloalkylene-Y₂—R₇;
a C₃-C₆heterocycloalkylene-Y₂—R₇ group, and
a heteroarylene-R₇ group optionally substituted by a linear or branched C₁-C₆alkyl group,
R₇ represents a group selected from the group consisting of: a linear or branched C₁-C₆alkyl group; a (C₃-C₆)cycloalkylene-R₈;

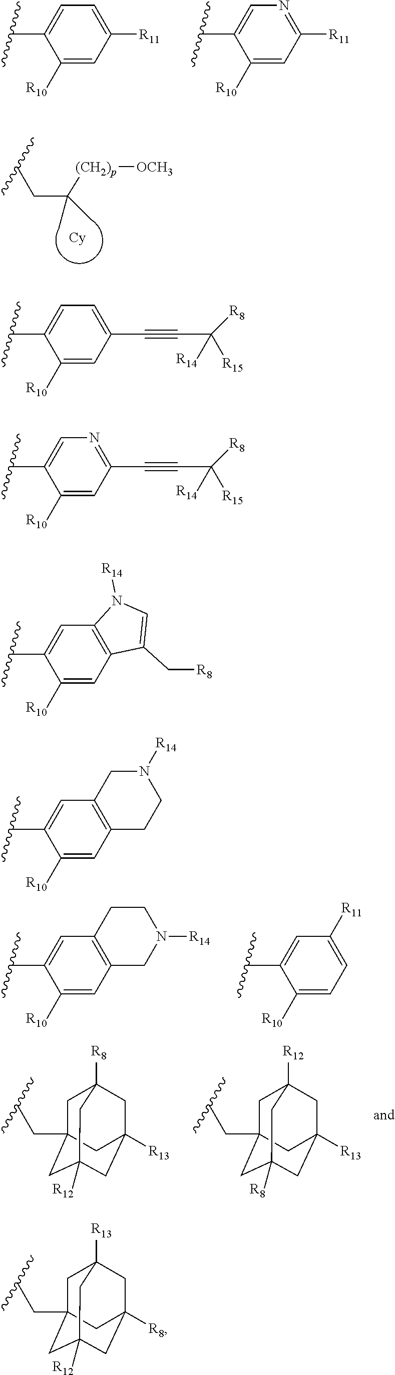

wherein Cy represents a C₃-C₈cycloalkyl,
R₈ represents a group selected from the group consisting of: hydrogen; a linear or branched C₁-C₆alkyl, —NR′ₐR′_{b}; —NR′ₐCO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}; —X′₂—NR′ₐR′_{b}; —NR′_{c}—X′₂—N₃ and
R₉ represents a group selected from the group consisting of a linear or branched C₁-C₆alkyl, trifluoromethyl, hydroxyl, halogen, and a C₁-C₆alkoxy,
R₁₀ represents a group selected from the group consisting of hydrogen, fluorine, chlorine, bromine, —CF₃ and methyl,
R₁₈ represents a group selected from the group consisting of hydrogen, a C₁-C₃alkylene-R₈, a —O—C₁-C₆alkylene-R₈, —CO—NRₕRᵢ and a —CH═CH—C₁-C₄alkylene-NRₕRᵢ, —CH═CH—CHO, a C₃-C₈cycloalkylene-CH₂—R₈, and a C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ and X₂ independently of one another, represent a linear or branched C₁-C₆alkylene group optionally substituted by one or two groups selected from the group consisting of trifluoromethyl, hydroxyl, a halogen, and a C₁-C₆alkoxy,
X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O—; a C₁-C₆alkylene-COOH; a C₁-C₆alkylene-PO(OH)₂; a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy group; and the group:

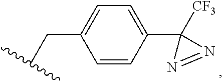 [SMILES: *Cc1ccc(C2(C(F)(F)F)N=N2)cc1]

or R′ₐ and R′_{b} form with the nitrogen atom carrying them a cycle B₃, or R′ₐ, R′_{b} and R′_{c} form with the nitrogen atom carrying them a bridged C₃-C₈ hetero cycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄, or R′_{d}, R′ₑ and R′_{f} form with the nitrogen atom carrying them a bridged C₃-C_{8 D}heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, or —NH—SO₂—,
m=0, 1 or 2,
B₁, B₂, B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl,
   wherein one of the R₃ and R₈ groups, if present, is covalently attached to the linker, and wherein the valency of an atom is not exceeded by virtue of one or more substituents bonded thereto; or

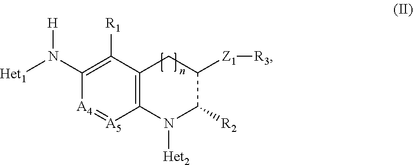

or an enantiomer, a diastereoisomer, and/or a pharmaceutically acceptable salt of any one of the foregoing, wherein:
n=0, 1 or 2,
------ represents a single or a double bond,
A₄ and A₅ independently of one another represent a carbon or a nitrogen atom,
Z₁ represents a bond, —N(R)—, or —O—, wherein R represents a hydrogen or a linear or branched C₁-C₆alkyl,
R₁ represents a group selected from the group consisting of: hydrogen; a linear or branched C₁-C₆alkyl optionally substituted by a hydroxyl or a C₁-C₆alkoxy group; a C₃-C₆cycloalkyl; trifluoromethyl; and a linear or branched C₁-C₆alkylene-heterocycloalkyl wherein the heterocycloalkyl group is optionally substituted by a linear or branched C₁-C₆alkyl group;
R₂ represents a hydrogen or a methyl;
R₃ represents a group selected from the group consisting of: hydrogen; a linear or branched C₁-C₄alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:
—X₁-≡C
Rₐ and R_{b} independently of one another represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl groups; a C₁-C₆alkylene-SO₂OH; a C₁-C₆alkylene-SO₂O—; a C₁-C₆alkylene-COOH; a C₁-C₆alkylene-PO(OH)₂; a C₁-C₆alkylene-NR_{d}Rₑ; a C₁-C₆alkylene-N⁺R_{d}RₑR_{f}; a C₁-C₆alkylene-phenyl wherein the phenyl may be substituted by a C₁-C₆alkoxy group; and the group:

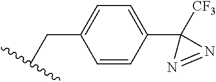 [SMILES: *Cc1ccc(C2(C(F)(F)F)N=N2)cc1]

or Rₐ and R_{b} form with the nitrogen atom carrying them a cycle B₁;
or Rₐ, R_{b} and R_{c} form with the nitrogen atom carrying them a bridged C₃-C₈ heterocycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a a cycle B₂, or R_{d}, Rₑ and R_{f} form with the nitrogen atom carrying them a bridged C₃-C₈ heterocycloalkyl,
Het₁ represents a group selected from the group consisting of:

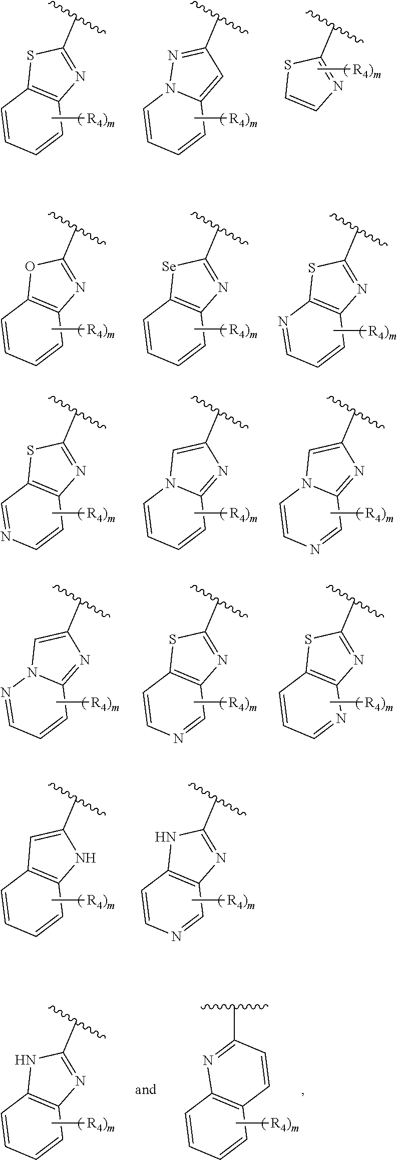

Het₂ represents a group selected from the group consisting of:

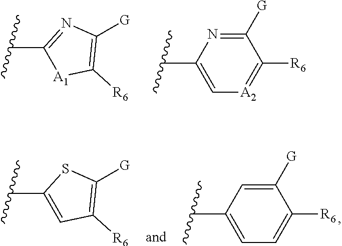

A₁ is —NH—, —N(C₁-C₆alkyl), O, S or Se,
A₂ is N, CH or C(R₅),
G is selected from the group consisting of:
   —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, —C₁-C₆alkyl optionally substituted by a hydroxyl group, halogen, —NO₂, and —CN, in which:
R_{G1} and R_{G2} at each occurrence are each independently selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂-C₆ alkynyl, a C₃-C₆cycloalkyl, phenyl and —(CH₂)₁₋₄-phenyl;
R_{G3} is selected from the group consisting of a C₁-C₆alkyl optionally substituted by 1 to 3 halogen atoms, a C₂-C₆alkenyl, a C₂-C₆alkynyl, a C₃-C₆cycloalkyl, phenyl and —(CH₂)₁₋₄-phenyl; or R_{G1} and R_{G2}, together with the atom to which each is attached are combined to form a C₃-C₆heterocycloalkyl; or in the alternative, G is selected from the group consisting of:

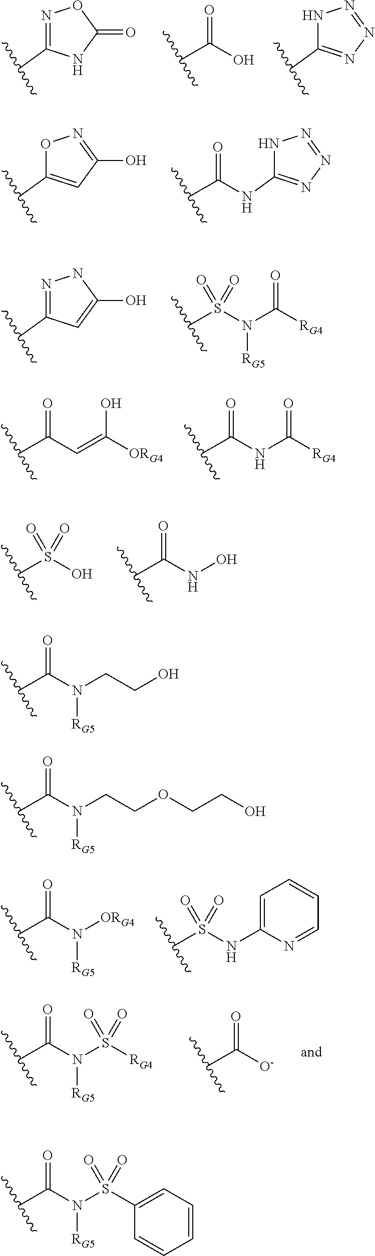

wherein R_{G4} is selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆ alkenyl, a C₂-C₆alkynyl and a C₃-C₆cycloalkyl, and R_{G5} represents a hydrogen atom or a C₁-C₆alkyl group optionally substituted by 1 to 3 halogen atoms,
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a C₂-C₆alkenyl; a C₂-C₆alkynyl; a halogen; and —CN,
R₆ represents a group selected from the group consisting of:
hydrogen;
a linear or branched —C₁-C₆alkylene-R₈ group;
a —C₂-C₆alkenyl;
—X₂—O—R⁷;

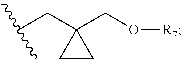

—X₂—NSO₂—R₇;
—C═C(R₉)—Y₁—O—R₇;
a C₃-C₆cycloalkyl;
a C₃-C₆heterocycloalkyl optionally substituted by a hydroxyl group;
a C₃-C₆cycloalkylene-Y₂—R₇;
a C₃-C₆heterocycloalkylene-Y₂—R₇ group, and
a heteroarylene-R₇ group optionally substituted by a linear or branched C₁-C₆alkyl group,
R₇ represents a group selected from the group consisting of: a linear or branched C₁-C₆alkyl group; a (C₃-C₆)cycloalkylene-R₈;

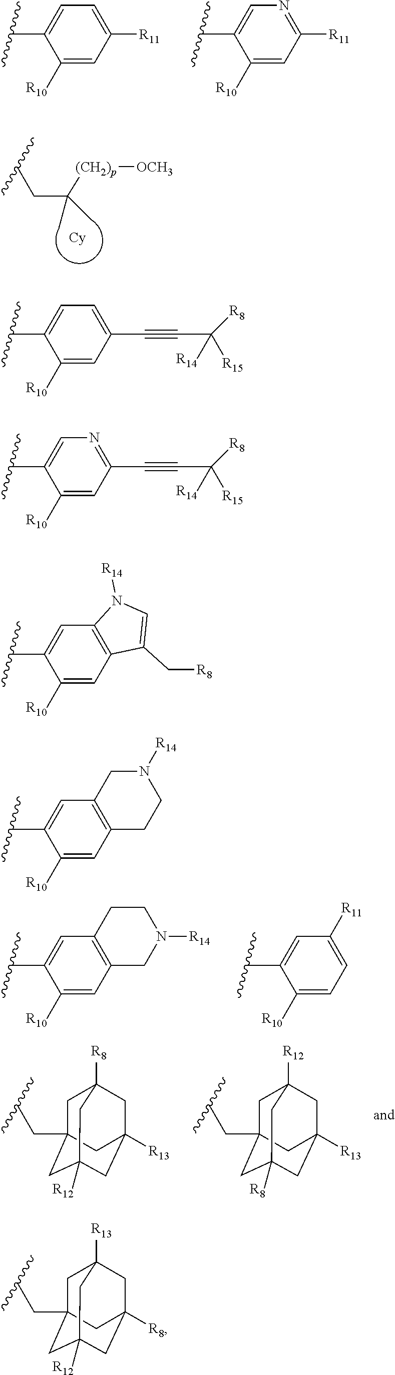

wherein Cy represents a C₃-C₈cycloalkyl,
R₈ represents a group selected from the group consisting of: hydrogen; a linear or branched C₁-C₆alkyl, —NR′ₐR′_{b}; —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}, —X′₂—NR′ₐR′_{b}, —NR′_{c}—X′₂—N₃ and:
   —NR′_{c}—X′₂-≡CH,
R₉ represents a group selected from the group consisting of a linear or branched C₁-C₆alkyl, trifluoromethyl, hydroxyl, a halogen, and a C₁-C₆alkoxy,
R₁₀ represents a group selected from the group consisting of hydrogen, fluorine, chlorine, bromine, —CF₃ and methyl,
R₁₁ represents a group selected from the group consisting of hydrogen, a halogen, a C₁-C₃alkylene-R₈, a —O—C₁-C₃alkylene-R₈, —CO—NRₕRᵢ and a —CH═CH—C₁-C₄alkylene-NRₕRᵢ, —CH═CH—CHO, a C₃-C₈cycloalkylene-CH₂—R₈, and a C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ represents a linear or branched C₁-C₄alkylene group optionally substituted by one or two groups selected from the group consisting of trifluoromethyl, hydroxyl, a halogen, and a C₁-C₆alkoxy,
X₂ represents a linear or branched C₁-C₆alkylene group optionally substituted by one or two groups selected from the group consisting of trifluoromethyl, hydroxyl, a halogen, and a C₁-C₆alkoxy,
X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O—; a C₁-C₆alkylene-COOH; a C₁-C₆alkylene-PO(OH)₂; a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy group; and the group:

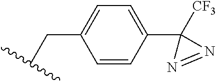 [SMILES: *Cc1ccc(C2(C(F)(F)F)N=N2)cc1]

or R′ₐ and R′_{b} form with the nitrogen atom carrying them a cycle B₃,
or R′ₐ, R′_{b} and R′_{c} form with the nitrogen atom carrying them a bridged C₃-C₈ heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄, or R′_{d}, R′ₑ and R′_{f} form with the nitrogen atom carrying them a bridged C₃-C₈ heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, or —NH—SO₂—,
m=0, 1 or 2,
B₁, B₂, B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl,
   wherein one of the R₃, R₈ and G groups, if present, is covalently attached to the linker, and wherein the valency of an atom is not exceeded by virtue of one or more substituents bonded thereto.

In a second embodiment of the present disclosure, PROTAC compounds of the present disclosure can be represented by Formula (A):

D-L-DSM (A),

or an enantiomer, a diastereoisomer, and/or a pharmaceutically acceptable salt of any one of the foregoing, wherein:
DSM is a degradation signaling compound (e.g., an E3 ubiquitin ligase recruitment ligand, such as a CRBN ligand or a VHL ligand) covalently attached to a linker L;
L is a linker that covalently attaches DSM to D; and
D is a Bcl-xL inhibitor compound of Formula (I) or Formula (II) covalently attached to the linker L:

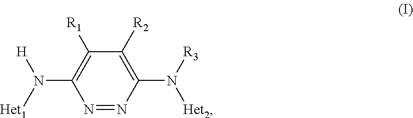

or an enantiomer, a diastereoisomer, and/or a pharmaceutically acceptable salt of any one of the foregoing, wherein:
R₁ and R₂ independently of one another represent a group selected from: hydrogen; linear or branched C₁-C₆alkyl optionally substituted by a hydroxyl or a C₁-C₆alkoxy group; C₃-C₆cycloalkyl; trifluoromethyl; linear or branched C₁-C₆alkylene-heterocycloalkyl wherein the heterocycloalkyl group is optionally substituted by a linear or branched C₁-C₆alkyl group;
   or R₁ and R₂ form with the carbon atoms carrying them a C₃-C₆cycloalkylene group,
R₃ represents a group selected from: hydrogen; C₃-C₆cycloalkyl; linear or branched C₁-C₆alkyl; —X₁—NRₐR_{b}; —X₁—N⁺RₐR_{b}R_{c}; —X₁—O—R₁; —X₁—COOR_{c}; —X₁—PO(OH)₂; —X₁—SO₂(OH); —X₁—N₃ and:
   —X₁-≡CH,
Rₐ and R_{b} independently of one another represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl groups; C₁-C₆alkylene-SO₂OH; C₁-C₆alkylene-SO₂O—; C₁-C₆alkylene-COOH; C₁-C₆alkylene-PO(OH)₂; C₁-C₆alkylene-NR_{d}Rₑ; C₁-C₆alkylene-N⁺R_{d}RₑR_{f}; C₁-C₆alkylene-phenyl wherein the phenyl may be substituted by a C₁-C₆alkoxy group; the group:

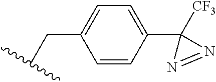 [SMILES: *Cc1ccc(C2(C(F)(F)F)N=N2)cc1]

or Rₐ and R_{b} form with the nitrogen atom carrying them a cycle B₁; or Rₐ, R_{b} and R_{c} form with the nitrogen atom carrying them a bridged C₃-C₈hetero cycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a a cycle B₂,
or R_{d}, Rₑ and R_{f} form with the nitrogen atom carrying them a bridged C₃-C₅hetero cycloalkyl,
Het₁ represents a group selected from:

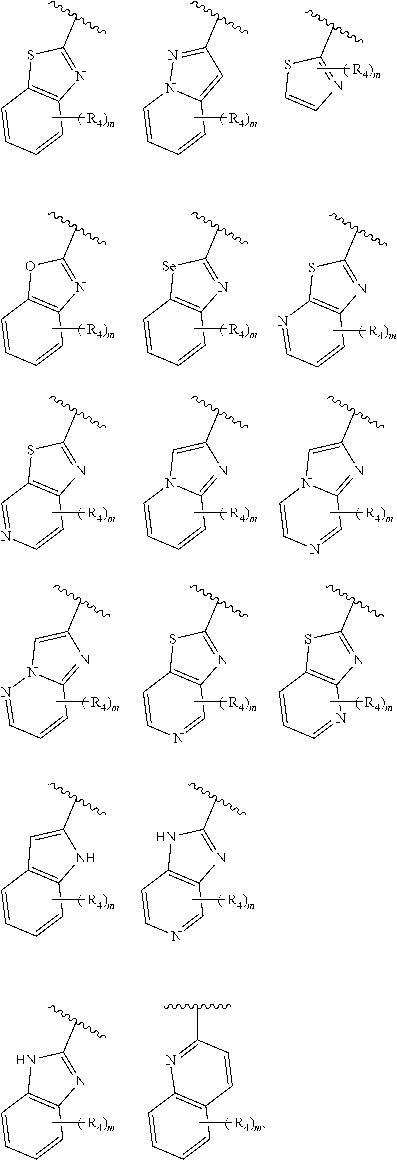

Het₂ represents a group selected from:

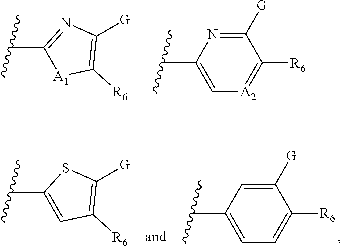

A₁ is —NH—, —N(C₁-C₃alkyl), O, S or Se,
A₂ is N, CH or C(R₅),
G is selected from the group consisting of: —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, halogen, —NO₂, and —CN, in which:
R_{G1} and R_{G2} at each occurrence are each independently selected from the group consisting of hydrogen, C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl;
R_{G3} is selected from the group consisting of C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl; or
   R_{G1} and R_{G2}, together with the atom to which each is attached are combined to form a C₃-C₈heterocycloalkyl; or in the alternative, G is selected from the group consisting of:

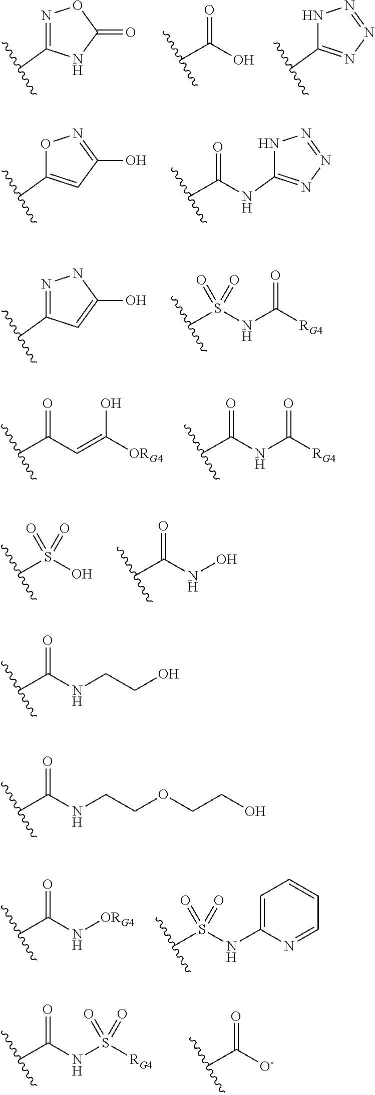

wherein R_{G4} is selected from C₁-C₆alkyl optionally substituted by 1 to 3 halogen atoms, C₂-C₆alkenyl, C₂-C₆alkynyl and C₃-C₆cycloalkyl,
R⁴ represents a hydrogen, fluorine, chlorine or bromine atom, a methyl, a hydroxyl or a methoxy group,
R₅ represents a group selected from: C₁-C₆alkyl optionally substituted by 1 to 3 halogen atoms; C₂-C₆alkenyl; C₂-C₆alkynyl; halogen or —CN,
R₆ represents a group selected from: hydrogen;
a linear or branched —C₁-C₆alkylene-R₈ group;
—C₂-C₆alkenyl;
—X₂—O—R⁷;

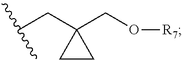

—X₂—NSO₂—R₇;
—C═C(R₉)—Y₁—O—R₇;
C₃-C₆cycloalkyl;
C₃-C₆heterocycloalkyl optionally substituted by a hydroxyl group;
C₃-C₆cycloalkylene-Y₂—R₇;
C₃-C₆heterocycloalkylene-Y₂—R₇ group,
a heteroarylene-R₇ group optionally substituted by a linear or branched C₁-C₆alkyl group,
R₇ represents a group selected from: linear or branched C₁-C₆alkyl group; (C₃-C₆)cycloalkylene-R₈; or:

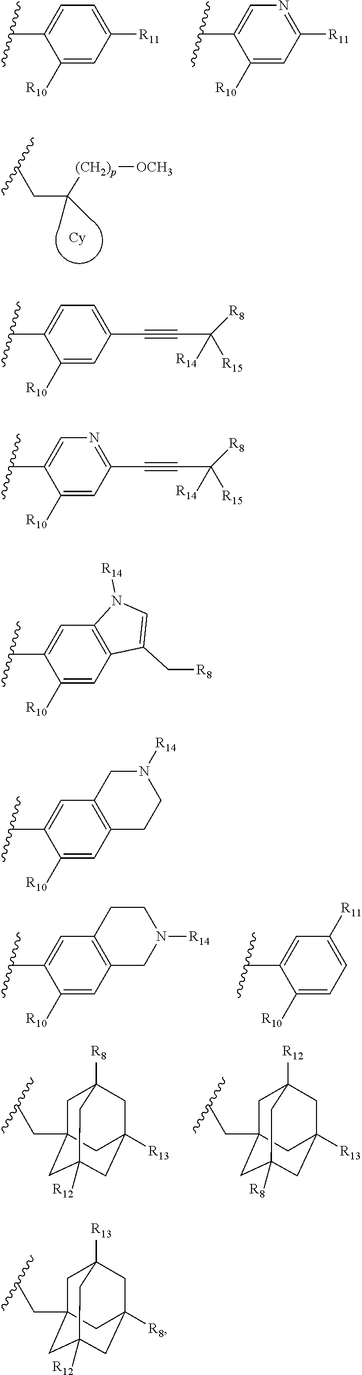

wherein Cy represents a C₃-C₈cycloalkyl,
R₈ represents a group selected from: hydrogen; linear or branched C₁-C₆alkyl, —NR′ₐR′_{b};
—NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}, —X′₂—NR′ₐR′_{b}, —NR′_{c}—X′₂—N₃ and: —NR′_{c}—X′₂-≡CH,
R₉ represents a group selected from linear or branched C₁-C₆alkyl, trifluoromethyl, hydroxyl, halogen, C₁-C₆alkoxy,
R₁₀ represents a group selected from hydrogen, fluorine, chlorine, bromine, —CF₃ and methyl,
R₁₁ represents a group selected from hydrogen, C₁-C₆alkylene-R₈, —O—C₁-C₃alkylene-R₈, —CO—NRₕRᵢ and —CH═CH—C₁-C₄alkylene-NRₕRᵢ, —CH═CH—CHO, C₃-C₈cycloalkylene-CH₂—R₈, C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ and X₂ independently of one another, represent a linear or branched C₁-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl or C₁-C₆alkoxy groups; C₁-C₆alkylene-SO₂OH; C₁-C₆alkylene-SO₂O—; C₁-C₆alkylene-COOH; C₁-C₆alkylene-PO(OH)₂; C₁-C₆alkylene-NR′_{d}R′ₑ; C₁-C₆alkylene-N⁺R′_{d}R′ₑR′_{f}; C₁-C₆alkylene-O—C₁-C₆alkylene-OH; C₁-C₆alkylene-phenyl wherein the phenyl may be substituted by a hydroxyl or a C₁-C₆alkoxy group; the group:

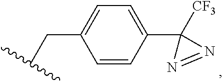 [SMILES: *Cc1ccc(C2(C(F)(F)F)N=N2)cc1]

or R′ₐ and R′_{b} form with the nitrogen atom carrying them a cycle B₃,
or R′ₐ, R′_{b} and R′_{c} form with the nitrogen atom carrying them a bridged C₃-C₈ hetero cycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈ heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, or —NH—SO₂—,
m=0, 1 or 2,
B₁, B₂, B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from: fluorine, bromine, chlorine, linear or branched C₁-C₆alkyl, hydroxyl, —NH₂, oxo or piperidinyl,
   wherein one of the R₃ and R₈ groups, if present, is covalently attached to the linker, and wherein the valency of an atom is not exceeded by virtue of one or more substituents bonded thereto; or

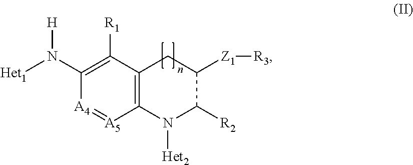

or an enantiomer, a diastereoisomer, and/or a pharmaceutically acceptable salt of any one of the foregoing, wherein:
n=0, 1 or 2,
------ represents a single or a double bond,
A₄ and A₅ independently of one another represent a carbon or a nitrogen atom,
Z₁ represents a bond, —N(R)—, or —O—, wherein R represents a hydrogen or a linear or branched C₁-C₆alkyl,
R₁ represents a group selected from: hydrogen; linear or branched C₁-C₆alkyl optionally substituted by a hydroxyl or a C₁-C₆alkoxy group; C₃-C₆cycloalkyl; trifluoromethyl; linear or branched C₁-C₆alkylene-heterocycloalkyl wherein the heterocycloalkyl group is optionally substituted by a a linear or branched C₁-C₆alkyl group;
R₂ represents a hydrogen or a methyl;
R₃ represents a group selected from: hydrogen; linear or branched C₁-C₄alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:
   —X₁-≡CH,
Rₐ and R_{b} independently of one another represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl groups; C₁-C₆alkylene-SO₂OH; C₁-C₆alkylene-SO₂O—; C₁-C₆alkylene-COOH; C₁-C₆alkylene-PO(OH)₂; C₁-C₆alkylene-NR_{d}Rₑ; C₁-C₆alkylene-N⁺R_{d}RₑR_{f}; C₁-C₆alkylene-phenyl wherein the phenyl may be substituted by a C₁-C₆alkoxy group; the group:

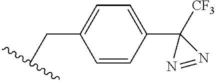 [SMILES: *Cc1ccc(C2(C(F)(F)F)N=N2)cc1]

or Rₐ and R_{b} form with the nitrogen atom carrying them a cycle B₁;
or Rₐ, R_{b} and R_{c} form with the nitrogen atom carrying them a bridged C₃-C₈ heterocycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a a cycle B₂, or R_{d}, Rₑ and R_{f} form with the nitrogen atom carrying them a bridged C₃-C₈ heterocycloalkyl,
Het₁ represents a group selected from:

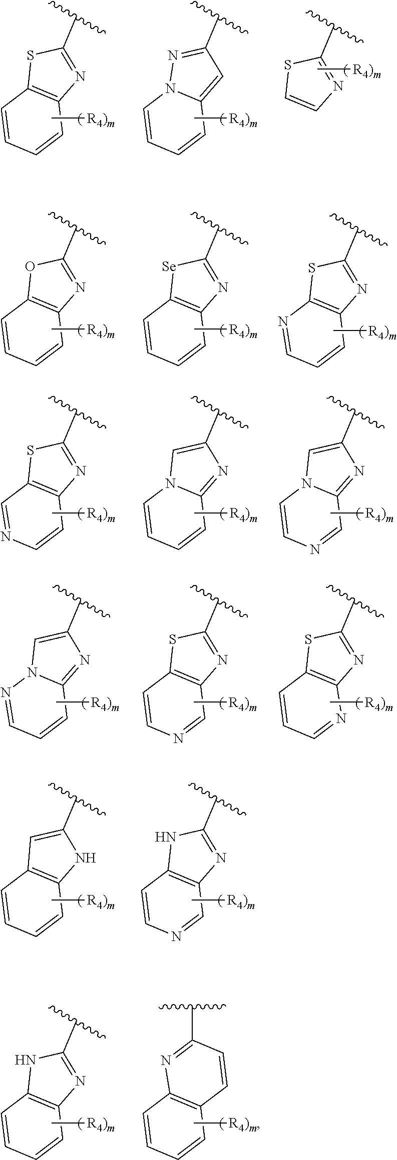

Het₂ represents a group selected from:

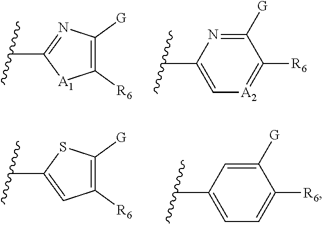

A₁ is —NH—, —N(C₁-C₃alkyl), O, S or Se,
A₂ is N, CH or C(R₅),
G is selected from the group consisting of:
   —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, halogen, —NO₂, and —CN, in which:
R_{G1} and R_{G2} at each occurrence are each independently selected from the group consisting of hydrogen, C₁-C₆alkyl optionally substituted by 1 to 3 halogen atoms, C₂-C₆alkenyl, C₂—C alkynyl, C₃-C₆cycloalkyl, phenyl and —(CH₂)₁₋₄-phenyl;
R_{G3} is selected from the group consisting of C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl; or R_{G1} and R_{G2}, together with the atom to which each is attached are combined to form a C₃-C₈heterocycloalkyl; or in the alternative, G is selected from the group consisting of:

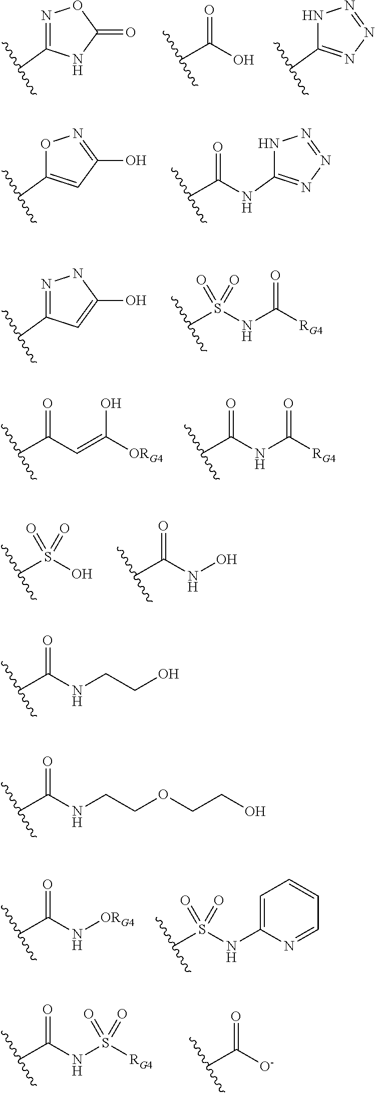

wherein R_{G4} is selected from C₁-C₆alkyl optionally substituted by 1 to 3 halogen atoms, C₂-C₆ alkenyl, C₂-C₆alkynyl and C₃-C₆cycloalkyl,
R₄ represents a hydrogen, fluorine, chlorine or bromine atom, a methyl, a hydroxyl or a methoxy group,
R₅ represents a group selected from: C₁-C₆alkyl optionally substituted by 1 to 3 halogen atoms; C₂-C₆alkenyl; C₂-C₆alkynyl; halogen or —CN,
R₆ represents a group selected from: hydrogen;
a linear or branched —C₁-C₆alkylene-R₈ group;
   —C₂-C₆alkenyl;
   —X₂—O—R⁷;

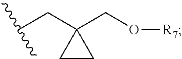

—X₂—NSO₂—R₇;
—C═C(R₉)—Y₁—O—R₇;
C₃-C₆cycloalkyl;
C₃-C₆heterocycloalkyl optionally substituted by a hydroxyl group;
C₃-C₆cycloalkylene-Y₂—R₇;
C₃-C₆heterocycloalkylene-Y₂—R₇ group,
a heteroarylene-R₇ group optionally substituted by a linear or branched C₁-C₆alkyl group,
R₇ represents a group selected from: linear or branched C₁-C₆alkyl group; (C₃-C₆)cycloalkylene-R₈; or:

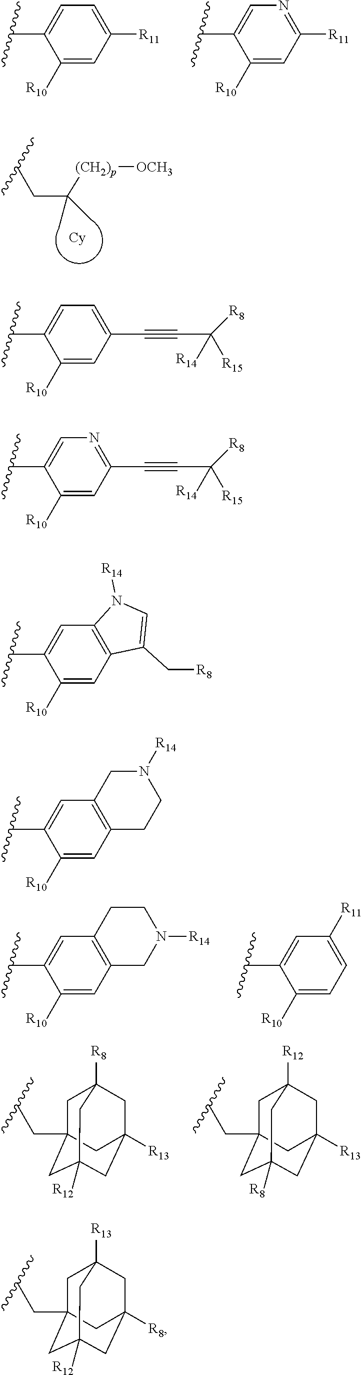

wherein Cy represents a C₃-C₈cycloalkyl,
R₈ represents a group selected from: hydrogen; linear or branched C₁-C₆alkyl, —NR′ₐR′_{b}; —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}, —X′₂—NR′ₐR′_{b}, —NR′_{c}—X′₂—N₃ and
   —NR′_{c}—X′₂-≡CH,
R₉ represents a group selected from linear or branched C₁-C₆alkyl, trifluoromethyl, hydroxyl, halogen, C₁-C₆alkoxy,
R₁₀ represents a group selected from hydrogen, fluorine, chlorine, bromine, —CF₃ and methyl,
R₁₁ represents a group selected from hydrogen, halogen, C₁-C₃alkylene-R₈, —O—C₁-C₃alkylene-R₈, —CO—NRₕRᵢ and —CH═CH—C₁-C₄alkylene-NRₕRᵢ, —CH═CH—CHO, C₃-C₈cycloalkylene-CH₂—R₈, C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ with the carbon atom carrying them form a cyclohexyl,
Rₕ and Rᵢ, independently of one another, represent a hydrogen or a linear or branched C₁-C₆alkyl group,
X₁ represents a linear or branched
C₁-C₄alkylene group optionally substituted by one or two groups selected from trifluoromethyl, hydroxyl, halogen, C₁-C₆alkoxy,
X₂ represents a linear or branched
C₁-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl or C₁-C₆alkoxy groups; C₁-C₆alkylene-SO₂OH; C₁-C₆alkylene-SO₂O⁻;
   C₁-C₆alkylene-COOH; C₁-C₆alkylene-PO(OH)₂; C₁-C₆alkylene-NR′_{d}R′ₑ; C₁-C₆alkylene-N⁺R′_{d}R′ₑR′_{f}; C₁-C₆alkylene-O—C₁-C₆alkylene-OH; C₁-C₆alkylene-phenyl
      wherein the phenyl may be substituted by a hydroxyl or a C₁-C₆alkoxy group; the group:

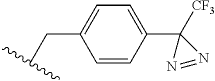 [SMILES: *Cc1ccc(C2(C(F)(F)F)N=N2)cc1]

or R′ₐ and R′_{b} form with the nitrogen atom carrying them a cycle B₃,
or R′ₐ, R′_{b} and R′_{c} form with the nitrogen atom carrying them a bridged C₃-C₈ heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈ heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, —NH—SO₂—,
m=0, 1 or 2,
B₁, B₂, B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from: fluorine, bromine, chlorine, linear or branched C₁-C₆alkyl, hydroxyl, —NH₂, oxo or piperidinyl,
   wherein one of the R₃ and R₈ groups, if present, is covalently attached to the linker, and wherein the valency of an atom is not exceeded by virtue of one or more substituents bonded thereto.

In a third embodiment of the present disclosure, for the compound of formula (A), or an enantiomer, a diastereomer, and/or a pharmaceutically acceptable salt thereof, the linker L comprises at least one group selected from the group consisting of: a linear or branched C₁-C₂₀alkylene optionally substituted by one to three groups selected from the group consisting of a C₁-C₆alkyl, a C₃-C₈cycloalkyl, trifluoromethyl, hydroxyl, a halogen, and a C₁-C₆alkoxy; a C₃-C₁₀cycloalkylene; a C₃-C₆heterocycloalkylene; —C(O)—; —O—; —S—; —N(R₁₆)—; —N(R₁₆)—C(O)—; —C(O)—N(R₁₆)—; —CH₂—C(O)—N(R₁₆)—; —N(R₁₆)—C(O)—CH₂—; a polyoxyethylene (PEG) group; an arylene group optionally substituted by one or two groups selected from the group consisting of a C₁-C₆alkyl, trifluoromethyl, hydroxyl, a halogen, and a C₁-C₆alkoxy; and a heteroarylene group, wherein R₁₆ represents hydrogen or C₁-C₆alkyl; and the remaining variables are as described in the first or second embodiment.

In a fourth embodiment of the present disclosure, for the compound of formula (A), or an enantiomer, a diastereomer, and/or a pharmaceutically acceptable salt thereof, the linker L of formula (A) comprises at least one group selected from: a linear or branched C₁-C₂₀alkylene optionally substituted by one or two groups selected from C₁-C₆alkyl, C₃-C₈cycloalkyl, trifluoromethyl, hydroxyl, halogen, and C₁-C₆alkoxy; a C₃-C₁₀cycloalkylene; —C(O)—; —O—; —S—; —N(R₁₆)—; —N(R₁₆)—C(O)—; —C(O)—N(R₁₆)—; —CH₂—C(O)—N(R₁₆)—; —N(R₁₆)—C(O)—CH₂—; a polyoxyethylene (PEG) group; an arylene group optionally substituted by one or two groups selected from C₁-C₆alkyl, trifluoromethyl, hydroxyl, halogen, and C₁-C₆alkoxy; and a heteroarylene group, wherein R₁₆ represents hydrogen or C₁-C₆alkyl; and the remaining variables are as described in the first or second embodiment.

In a fifth embodiment of the present disclosure, for the compound of formula (A), or the enantiomer, the diastereomer, and/or the pharmaceutically acceptable salt thereof, the linker L of formula (A) comprises a 1,2,3-triazolene group formed by reacting an azide-containing precursor with an alkyne-containing precursor; and the remaining variables are as described in the first, second, third or fourth embodiment.

In a sixth embodiment of the present disclosure, for the compound of formula (A), or an enantiomer, a diastereomer, and/or a pharmaceutically acceptable salt thereof, -L- is represented by formula (i), (ii), (iii), (iv), (v), (vi) or (vii):

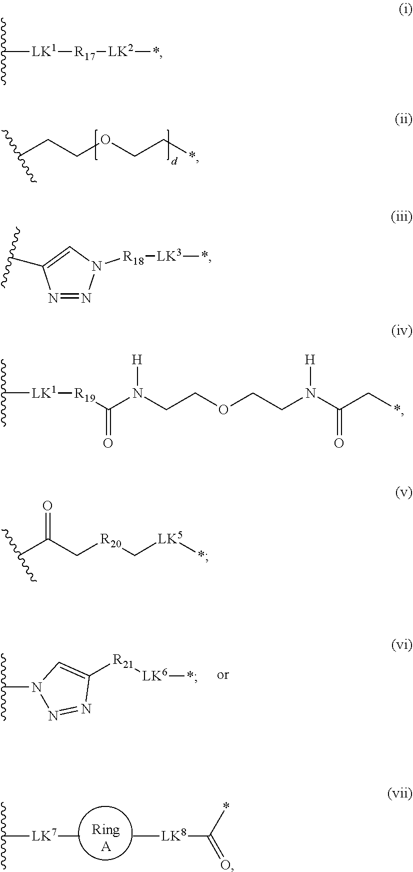

wherein:
LK¹ is a bond, —NR¹⁶— or —C(O)—;
LK² is a bond, —C(O)— or —N(R₁₆)—C(O)—CH₂—*;
LK³ is —C(O)— or —N(R₁₆)—C(O)—CH₂—*;
LK⁴ is a bond or —C(O)—;
LK⁵ is a bond or —C(O)—;
LK⁶ is a bond, —C(O)—, —O—CH₂—C(O)—*, or —N(R₁₆)—C(O)—CH₂—*;
LK⁷ is a bond or —NR¹⁶—;
LK⁸ is a bond, —R₂₂—, —O—R₂₂— or —C(O)—R₂₂—;
Ring A is a C₃-C₈ heterocyloalkylene;
R₁₆ is H or methyl;
R₁₇ is a C₁-C₂₀alkylene, a C₃₋₁₀cycloalkylene, a C₃₋₁₀cycloalkylene-CH₂—**, phenylene, —C₁-C₂₀alkylene-OCH₂CH₂—**, —C₁-C₂₀alkylene-OCH₂—**, —CH₂—(OCH₂CH₂)ₚ—OCH₂—** or —(CH₂CH₂O)ₚ—(C₁-C₃alkylene)-**, wherein the C₁-C₂₀alkylene or the phenylene is optionally substituted with one or two R₁₇ₐ; and ** indicates the attachment point to LK²;
R₁₇ₐ, for each occurrence, is independently a linear or branched C₁-C₆alkyl or a halogen, or two R₁₇ₐ together with the carbon atom from which they are attached form a C₃-C₆cycloalkyl;
R₁₈ is a C₁-C₂₀alkylene or —CH₂CH₂—(OCH₂CH₂)ₚ—**, wherein ** indicates the attachment point to LK³;
R₁₉ is a C₁-C₆alkylene;
R₂₀ is a C₃-C₁₀cycloalkylene, phenylene, —S— or —N(R₁₆)—;
R₂₁ is a C₁-C₂₀alkylene or —CH₂—(OCH₂CH₂)ₚ—**, wherein ** indicates the attachment point to LK⁶;
R₂₂ is a C₁-C₆alkylene;
p is an integer from 1 to 7;
d is an integer from 1 to 7;

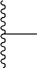

is a bond to the Bcl-xL inhibitor compound; and
-* is a bond to DSM;
   and the remaining variables are as described in the first, second, third or second embodiment.

In a seventh embodiment of the present disclosure, for the compound of formula (A), or an enantiomer, a diastereomer, and/or a pharmaceutically acceptable salt thereof, the linker L of formula (A) is represented by formula (i), (ii), (iii), (iv), (v), or (vi):

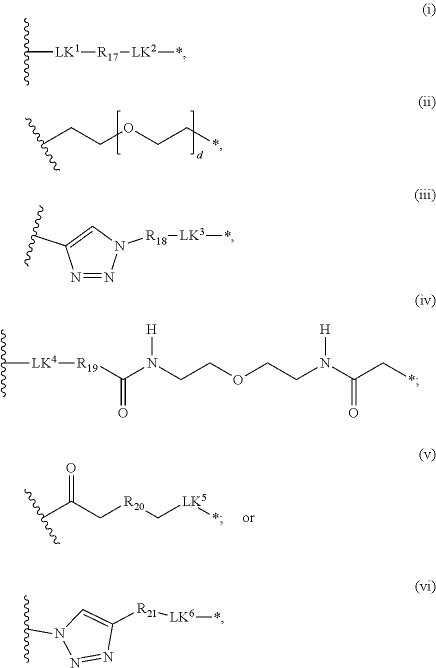

wherein:
LK¹ is a bond or —C(O)—;
LK² is a bond, —C(O)— or —N(R₁₆)—C(O)—CH₂—*;
LK³ is —C(O)— or —N(R₁₆)—C(O)—CH₂—*;
LK⁴ is a bond or —C(O)—;
LK⁵ is a bond or —C(O)—;
LK⁶ is a bond, —O—CH₂—C(O)—*, or —N(R₁₆)—C(O)—CH₂—*;
R₁₆ is H or methyl;
R₁₇ is C₁-C₂₀alkylene, C₃₋₁₀cycloalkylene, phenylene, —CH₂—(OCH₂CH₂)ₚ—OCH₂—, wherein C₁-C₁₅alkylene or phenylene is optionally substituted with one or two R₁₇ₐ;
R₁₇ₐ, for each occurrence, is independently a linear or branched C₁₋₆alkyl or a halogen, or two R₁₇ₐ together with the carbon atom from which they are attached form a C₃₋₆ cycloalkyl;
R₁₈ is C₁₋₂₀alkylene or —CH₂CH₂—(OCH₂CH₂)ₚ—**, wherein ** indicates the attachment point to LK³;
R₁₉ is C₁₋₆alkylene;
R₂₀ is C₃₋₁₀cycloalkylene, phenylene, —S— or —N(R₁₆)—;
R₂₁ is C₁₋₂₀alkylene or —CH₂—(OCH₂CH₂)ₚ—**, wherein ** indicates the attachment point to LK⁶;
p is an integer from 1 to 7;
d is an integer from 1 to 7;

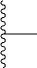

is a bond to the Bcl-xL inhibitor compound; and
-* is a bond to DSM;
   and the remaining variables are as described in the first, second, third or fourth embodiment.

In an eighth embodiment of the present disclosure, for the compound of formula (A), or an enantiomer, a diastereomer, and/or a pharmaceutically acceptable salt thereof, the linker L of formula (A) is selected from:

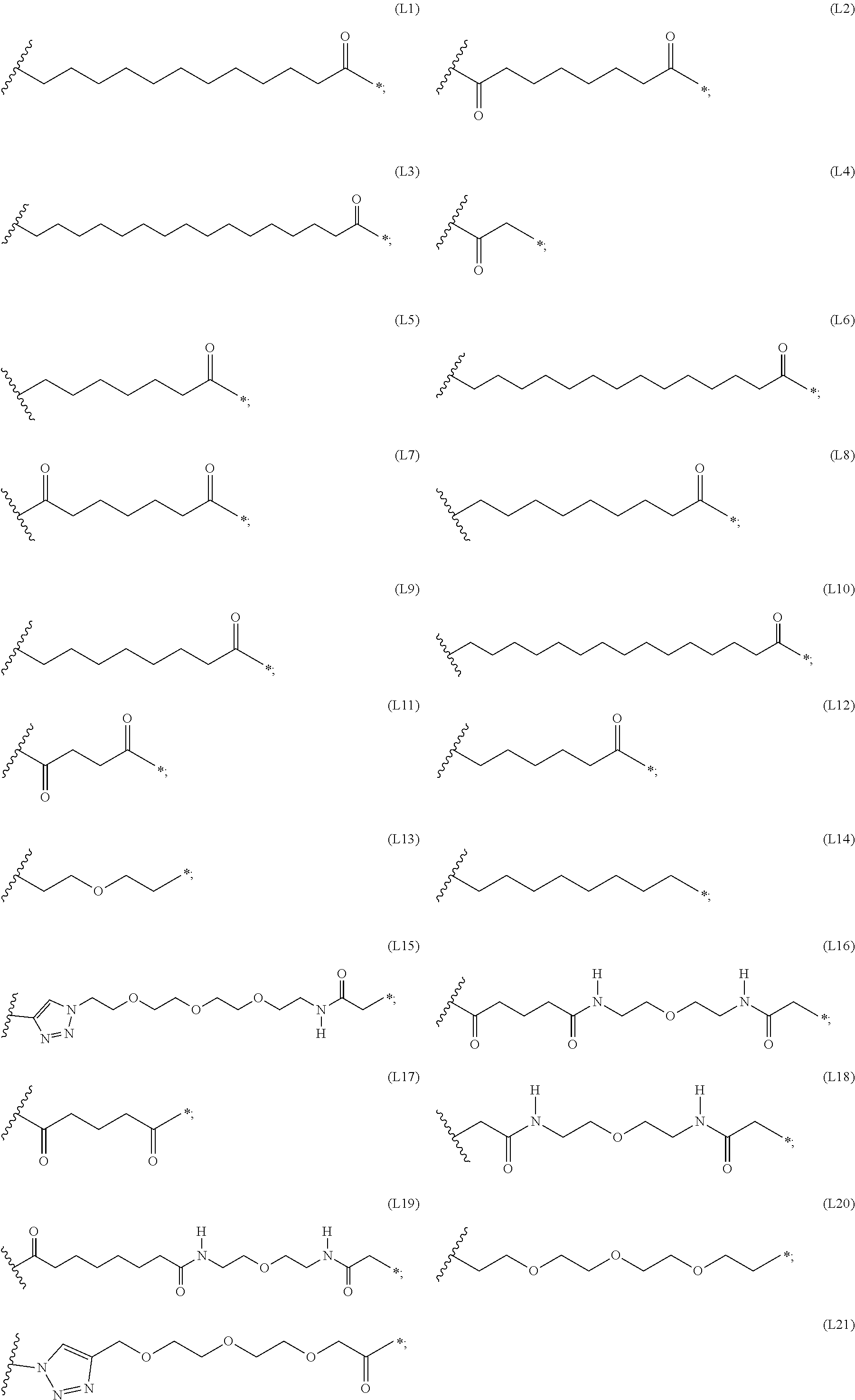

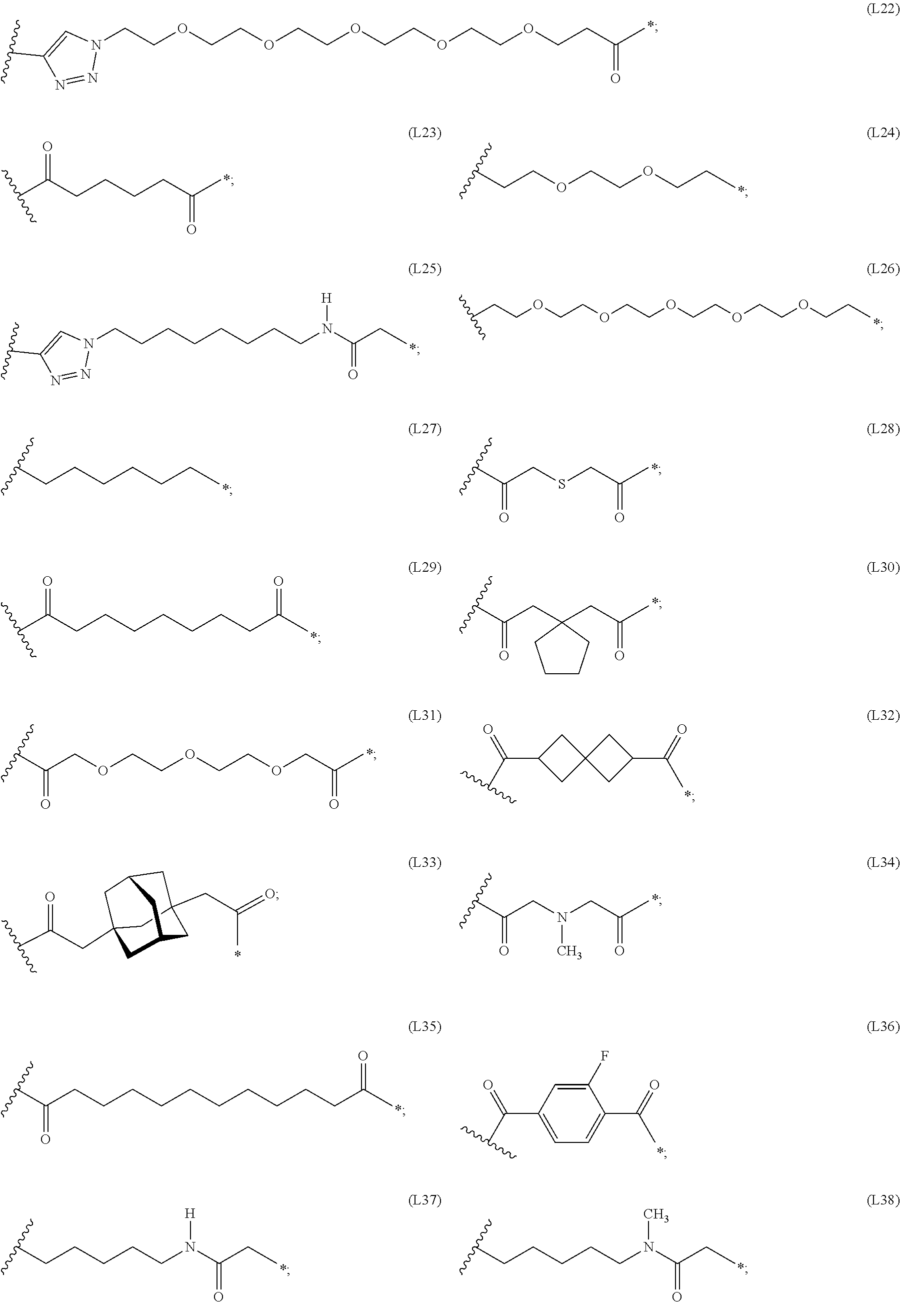

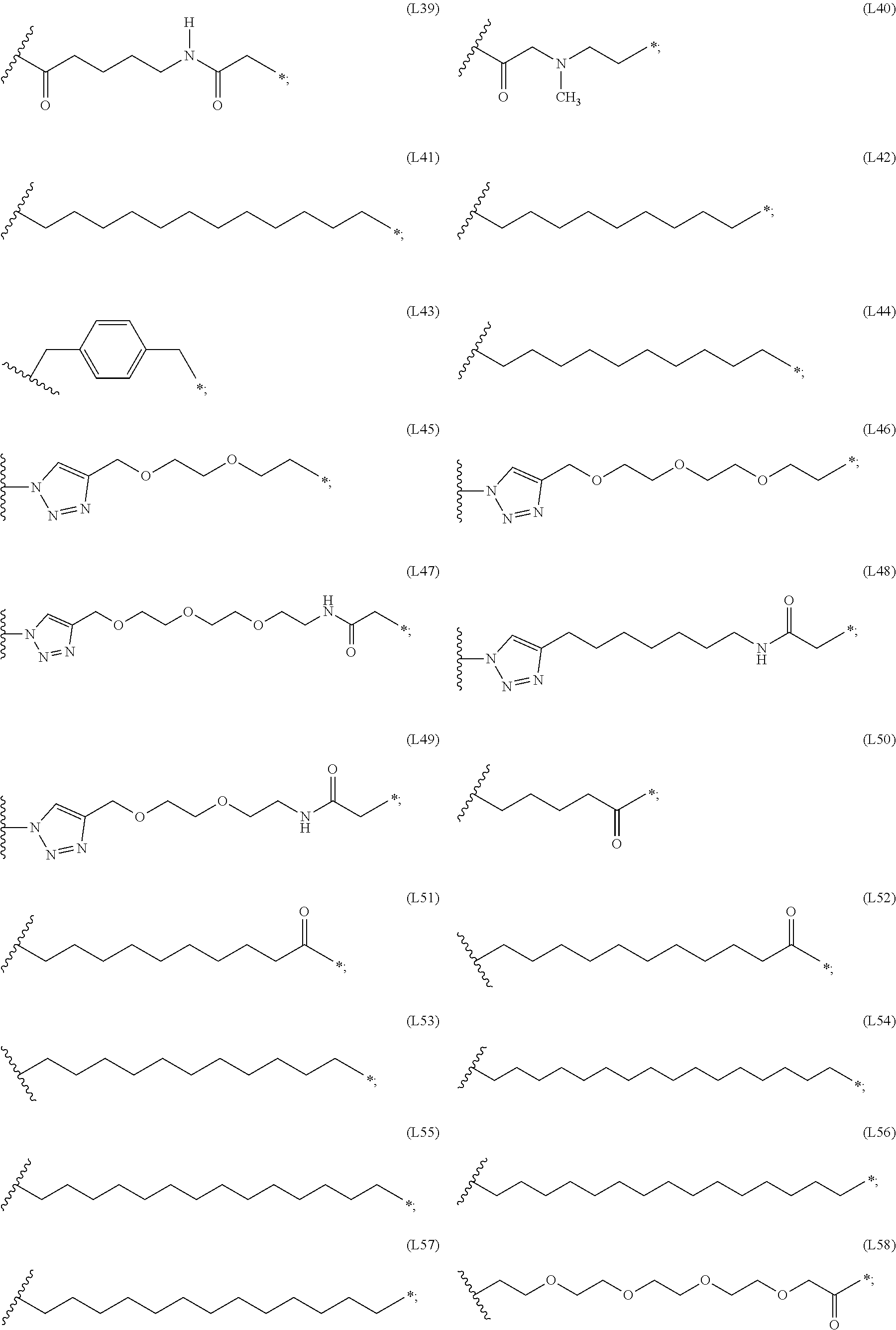

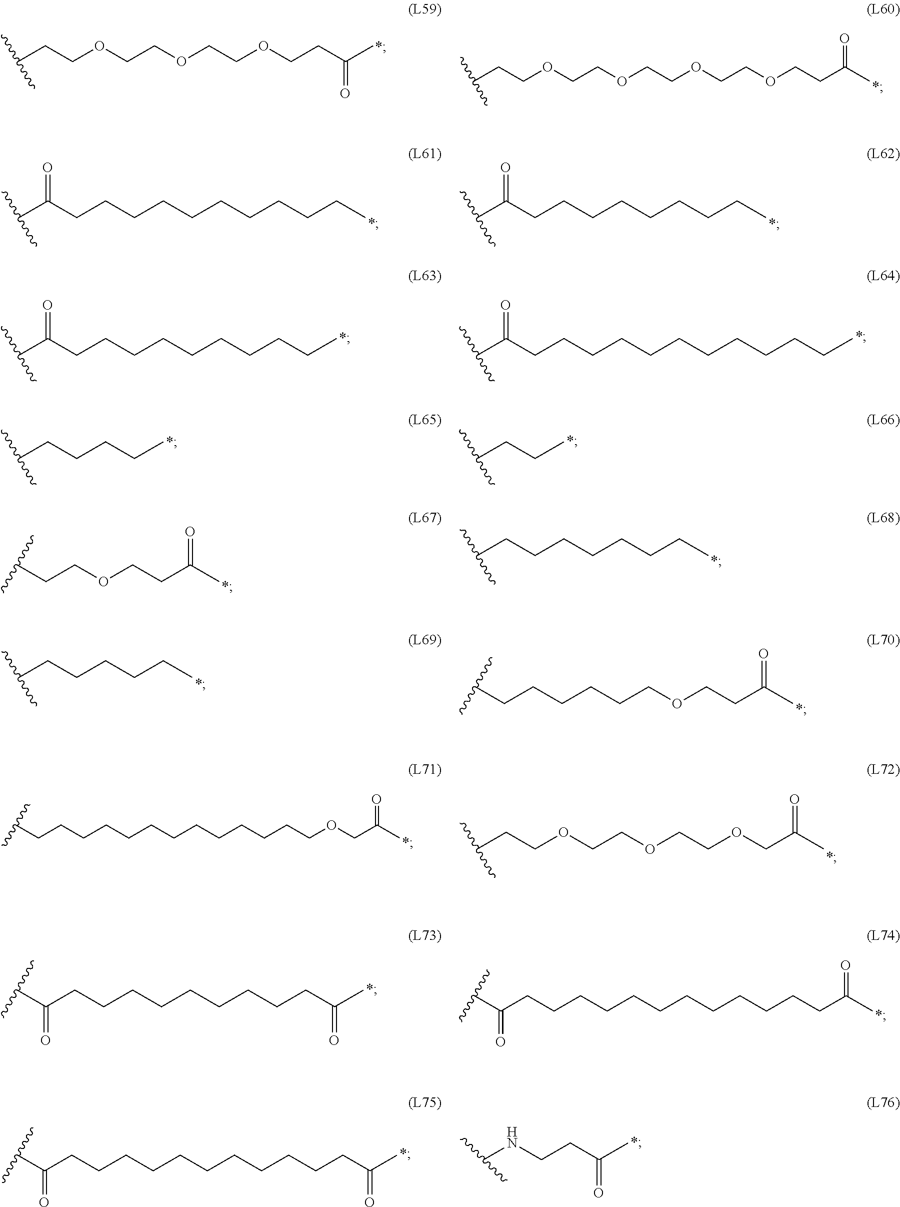

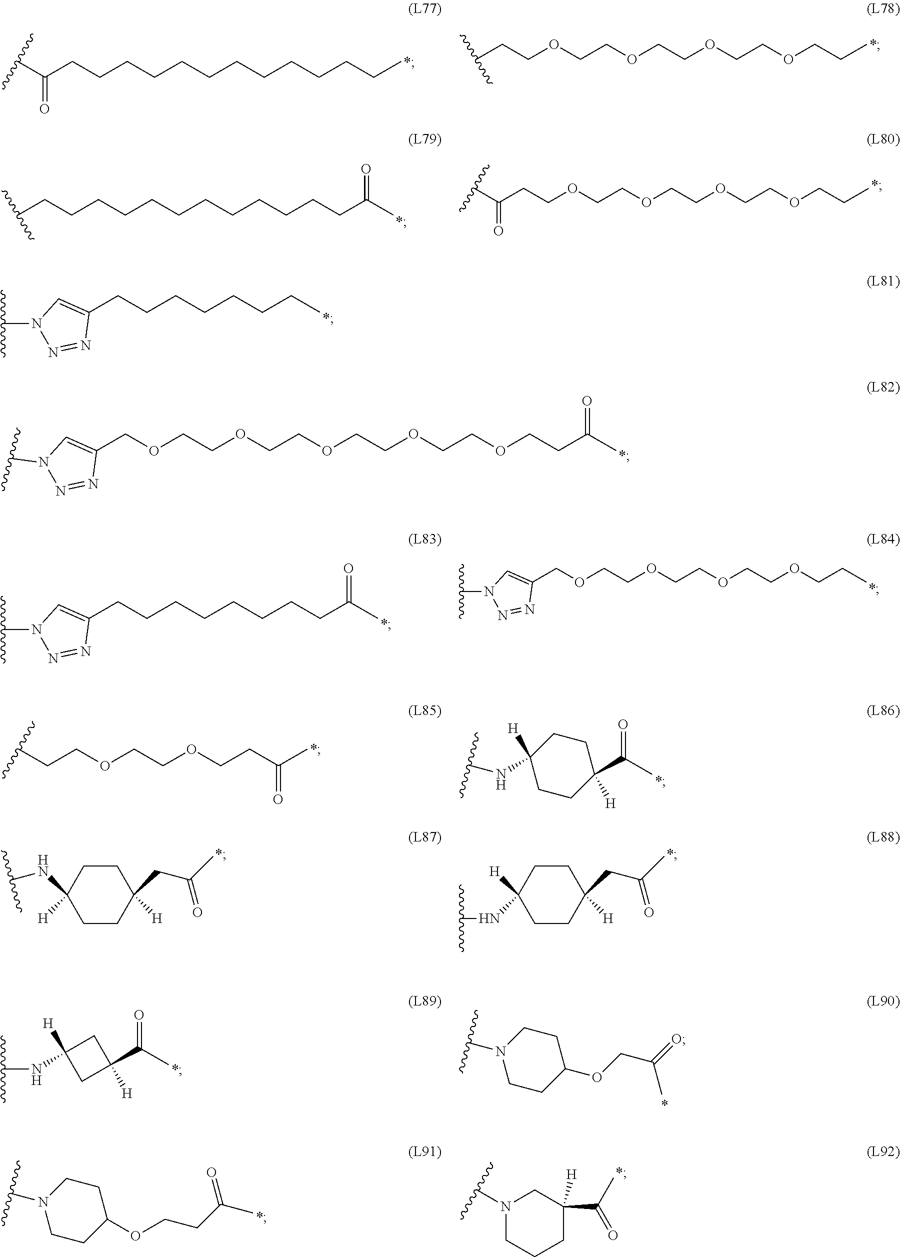

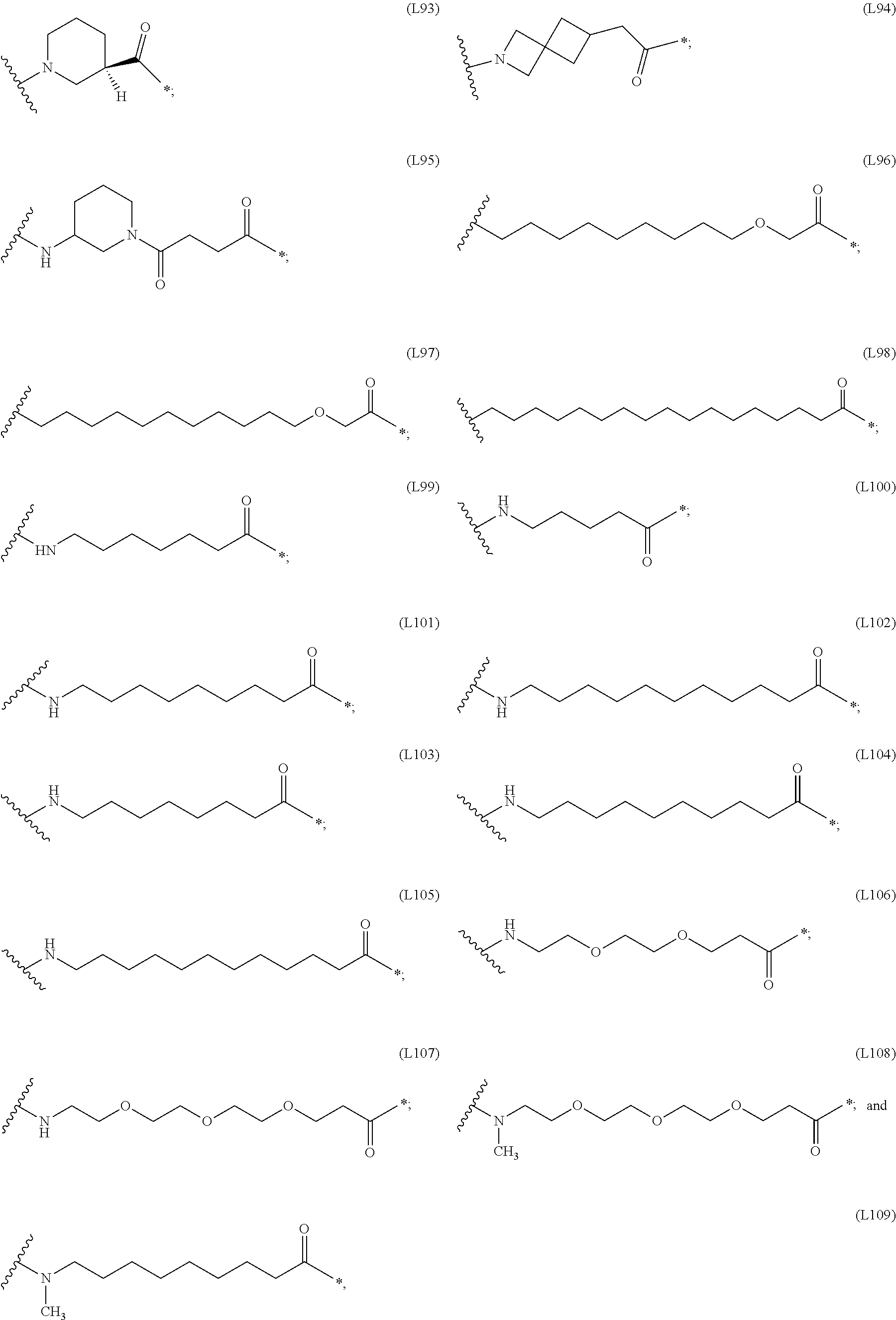

wherein:

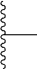

is a bond to the Bcl-xL inhibitor compound; and
-* is a bond to DSM;
   and the remaining variables are as described in the first, second, third or fourth embodiment. In some embodiments, the linker L of formula (A) is selected from (L1)-(L50).

In a ninth embodiment of the present disclosure, for the compound of formula (A), or an enantiomer, a diastereomer, and/or a pharmaceutically acceptable salt thereof, D comprises a compound of Formula (I):

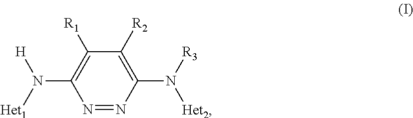

or an enantiomer, a diastereoisomer, and/or a pharmaceutically acceptable salt of any one of the foregoing, wherein:
R₁ and R₂ independently of one another represent a group selected from the group consisting of: hydrogen; a linear or branched C₁-C₆alkyl optionally substituted by a hydroxyl or a C₁-C₆alkoxy group; a C₃-C₆cycloalkyl; trifluoromethyl; and a linear or branched C₁-C₆alkylene-heterocycloalkyl wherein the heterocycloalkyl group is optionally substituted by a linear or branched C₁-C₆alkyl group; or R₁ and R₂ form with the carbon atoms carrying them a C₃-C₆cycloalkylene group,
R₃ represents a group selected from the group consisting of: hydrogen; a C₃-C₆cycloalkyl; a linear or branched C₁-C₆alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:
   —X₁-≡CH,
Rₐ and R_{b} independently of one another represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl groups; a C₁-C₆alkylene-SO₂OH; a C₁-C₆alkylene-SO₂O—; a C₁-C₆alkylene-COOH; a C₁-C₆alkylene-PO(OH)₂; a C₁-C₆alkylene-NR_{d}Rₑ; a C₁-C₆alkylene-N⁺R_{d}RₑR_{f}; a C₁-C₆alkylene-phenyl wherein the phenyl may be substituted by a C₁-C₆alkoxy group; and the group:

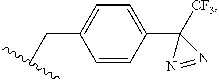 [SMILES: *Cc1ccc(C2(C(F)(F)F)N=N2)cc1]

or Rₐ and R_{b} form with the nitrogen atom carrying them a cycle B₁; or Rₐ, R_{b} and R_{c} form with the nitrogen atom carrying them a bridged C₃-C₈hetero cycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a a cycle B₂,
or R_{d}, Rₑ and R_{f} form with the nitrogen atom carrying them a bridged C₃-C₆hetero cycloalkyl,
Het₁ represents a group selected from the group consisting of:

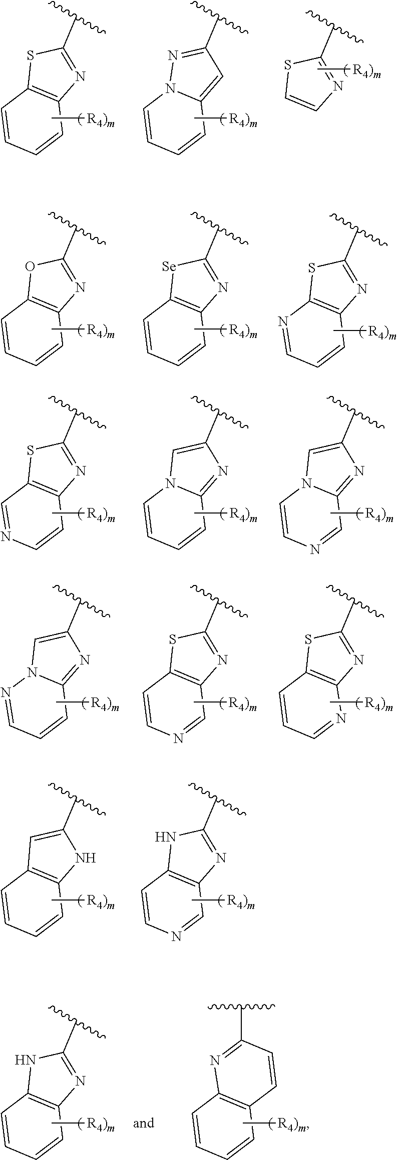

Het₂ represents a group selected from the group consisting of:

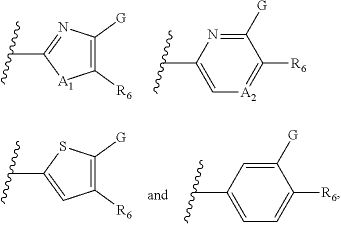

A₁ is —NH—, —N(C₁-C₆alkyl), O, S or Se,
A₂ is N, OH or C(R₅),
G is selected from the group consisting of:
   —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, —C₁-C₆alkyl optionally substituted by a hydroxyl group, a halogen, —NO₂, and —CN, in which:
R_{G1} and R_{G2} at each occurrence are each independently selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂-C₆alkynyl, a C₃-C₆cycloalkyl, phenyl and —(CH₂)₁₋₄-phenyl;
R_{G3} is selected from the group consisting of a C₁-C₆alkyl optionally substituted by 1 to 3 halogen atoms, a C₂-C₆alkenyl, a C₂-C₆alkynyl, a C₃-C₆cycloalkyl, phenyl and —(CH₂)₁₋₄-phenyl; or R_{G1} and R_{G2}, together with the atom to which each is attached are combined to form a C₃-C₈heterocycloalkyl; or in the alternative, G is selected from the group consisting of:

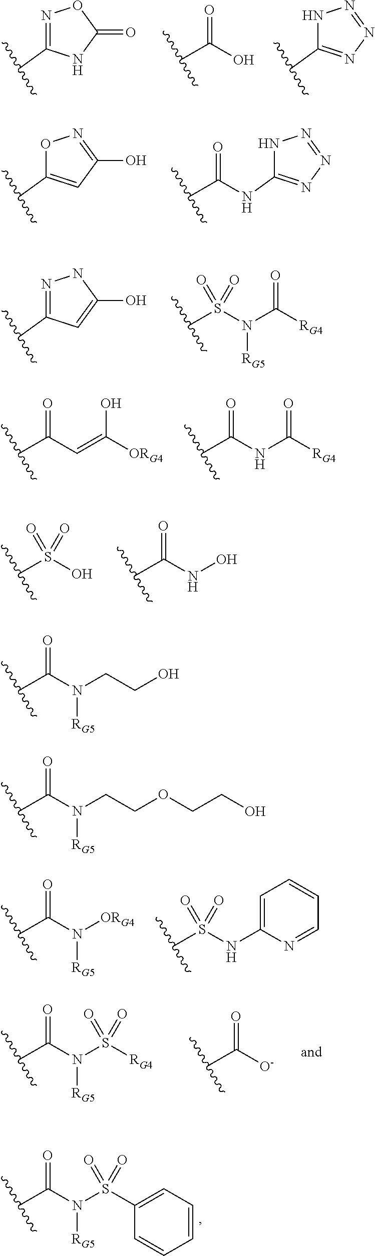

wherein R_{G4} is selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂-C₆alkynyl and a C₃-C₆cycloalkyl, and R_{G5} represents a hydrogen atom or a C₁-C₆alkyl group optionally substituted by 1 to 3 halogen atoms,
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a C₂-C₆alkenyl; a C₂-C₆alkynyl; a halogen; and —CN,
R₆ represents a group selected from the group consisting of:
hydrogen;
a linear or branched —C₁-C₆alkylene-R₈ group;
a —C₂-C₆alkenyl;
   —X₂—O—R⁷;

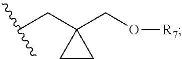

X₂—NSO₂—R₇;
—C═C(R₉)—Y₁—O—R₇;
a C₃-C₆cycloalkyl;
a C₃-C₆heterocycloalkyl optionally substituted by a hydroxyl group;
a C₃-C₆cycloalkylene-Y₂—R₇;
a C₃-C₆heterocycloalkylene-Y₂—R₇ group, and
a heteroarylene-R₇ group optionally substituted by a linear or branched C₁-C₆alkyl group,
R₇ represents a group selected from the group consisting of: a linear or branched C₁-C₆alkyl group; a (C₃-C₆)cycloalkylene-R₈;

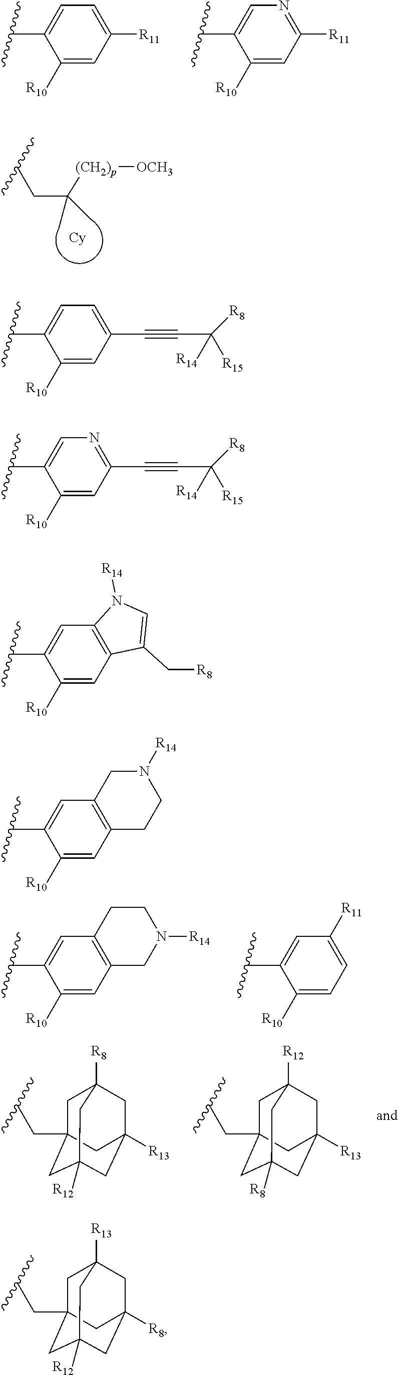

wherein Cy represents a C₃-C₈cycloalkyl,
R₈ represents a group selected from the group consisting of: hydrogen; linear or branched C₁-C₆alkyl, —NR′ₐR′_{b}; —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}; —X′₂—NR′ₐR′_{b}; —NR′_{c}—X′₂—N₃ and:
   —NR′_{c}—X′₂-≡CH,
R₉ represents a group selected from the group consisting of a linear or branched C₁-C₆alkyl, trifluoromethyl, hydroxyl, a halogen, and a C₁-C₆alkoxy,
R₁₀ represents a group selected from the group consisting of hydrogen, fluorine, chlorine, bromine, —CF₃ and methyl,
R₁₁ represents a group selected from the group consisting of hydrogen, a C₁-C₃alkylene-R₈, a —O—C₁-C₃alkylene-R₈, —CO—NRₕRᵢ and a —CH═CH—C₁-C₄alkylene-NRₕRᵢ, —CH═CH—CHO, a C₃-C₈cycloalkylene-CH₂—R₈, and a C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ and X₂ independently of one another, represent a linear or branched C₁-C₆alkylene group optionally substituted by one or two groups selected from the group consisting of trifluoromethyl, hydroxyl, a halogen, and a C₁-C₆alkoxy,
X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O—; a C₁-C₆alkylene-COOH; a C₁-C₆alkylene-PO(OH)₂; a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy group; and the group:

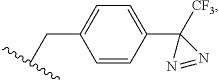 [SMILES: *Cc1ccc(C2(C(F)(F)F)N=N2)cc1]

or R′ₐ and R′_{b} form with the nitrogen atom carrying them a cycle B₃,
or R′ₐ, R′_{b} and R′_{c} form with the nitrogen atom carrying them a bridged C₃-C₈ hetero cycloalkyl,
R′_{c}, R′_{d}, R′ₑ, R′_{f}, independently of one another, represents a hydrogen or a linear or branched C₁-C₆alkyl group,
   or R′_{d} and R′ₑ form with the nitrogen atom carrying them a cycle B₄,
or R′_{d}, R′ₑ and R′_{f} form with the nitrogen atom carrying them a bridged C₃-C₈ heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, or —NH—SO₂—,
m=0, 1 or 2,
B₁, B₂, B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl,
   wherein one of the R₃ and R₈ groups, if present, is covalently attached to the linker, and wherein the valency of an atom is not exceeded by virtue of one or more substituents bonded thereto; and the remaining variables are as described in the first, second, third, fourth, fifth, sixth, seventh or eighth embodiment.

In a tenth embodiment of the present disclosure, for the compound of formula (A), or an enantiomer, a diastereomer, and/or a pharmaceutically acceptable salt thereof, D in Formula (A) comprises a compound of Formula (I):

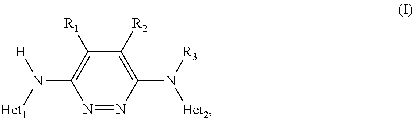

wherein:
R₁ and R₂ independently of one another represent a group selected from: hydrogen; linear or branched C₁-C₆alkyl optionally substituted by a hydroxyl or a C₁-C₆alkoxy group; C₃-C₆cycloalkyl; trifluoromethyl; linear or branched C₁-C₆alkylene-heterocycloalkyl wherein the heterocycloalkyl group is optionally substituted by a a linear or branched C₁-C₆alkyl group; or R₁ and R₂ form with the carbon atoms carrying them a C₃-C₆cycloalkylene group,
R₃ represents a group selected from: hydrogen; C₃-C₆cycloalkyl; linear or branched C₁-C₆alkyl; —X₁—NRₐR_{b}; —X₁—N⁺RₐR_{b}R_{c}; —X₁—O—R₁; —X₁—COOR_{c}; —X₁—PO(OH)₂; —X₁—SO₂(OH); —X₁—N₃ and:
   —X₁-≡CH,
Rₐ and R_{b} independently of one another represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl groups; C₁-C₆alkylene-SO₂OH; C₁-C₆alkylene-SO₂O—; C₁-C₆alkylene-COOH; C₁-C₆alkylene-PO(OH)₂; C₁-C₆alkylene-NR_{d}Rₑ; C₁-C₆alkylene-N⁺R_{d}RₑR_{f}; C₁-C₆alkylene-phenyl wherein the phenyl may be substituted by a C₁-C₆alkoxy group; the group:

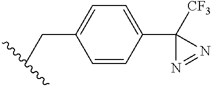 [SMILES: *Cc1ccc(C2(C(F)(F)F)N=N2)cc1]

or Rₐ and R_{b} form with the nitrogen atom carrying them a cycle B₁; or Rₐ, R_{b} and R_{c} form with the nitrogen atom carrying them a bridged C₃-C₈hetero cycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a a cycle B₂, or R_{d}, Rₑ and R_{f} form with the nitrogen atom carrying them a bridged C₃-C₅hetero cycloalkyl,
Het₁ represents a group selected from:

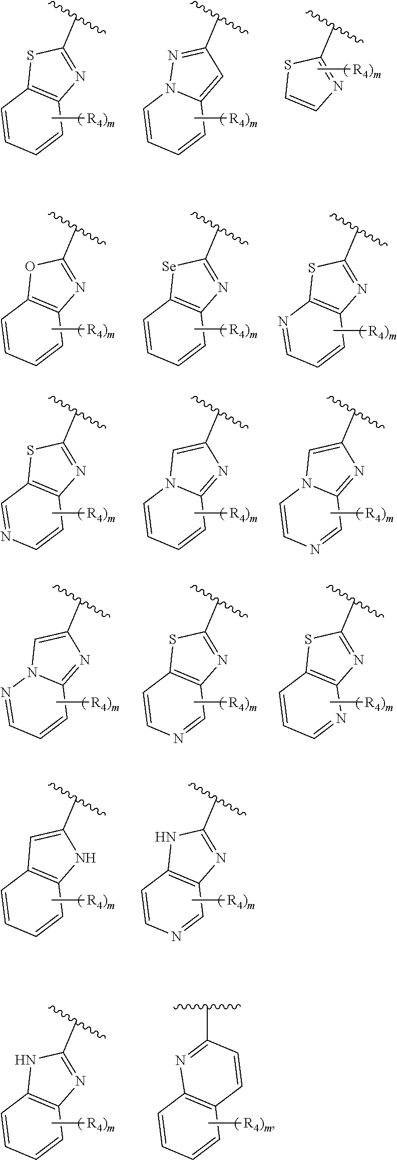

Het₂ represents a group selected from:

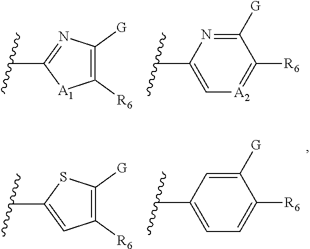

A₁ is —NH—, —N(C₁-C₃ alkyl), O, S or Se,
A₂ is N, CH or C(R₅),
G is selected from the group consisting of:
   —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2},
   —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, halogen, —NO₂, and —CN, in which:
R_{G1} and R_{G2} at each occurrence are each independently selected from the group consisting of hydrogen, C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl;
R_{G3} is selected from the group consisting of C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl; or R_{G1} and R_{G2}, together with the atom to which each is attached are combined to form a C₃-C₈heterocycloalkyl; or in the alternative, G is selected from the group consisting of:

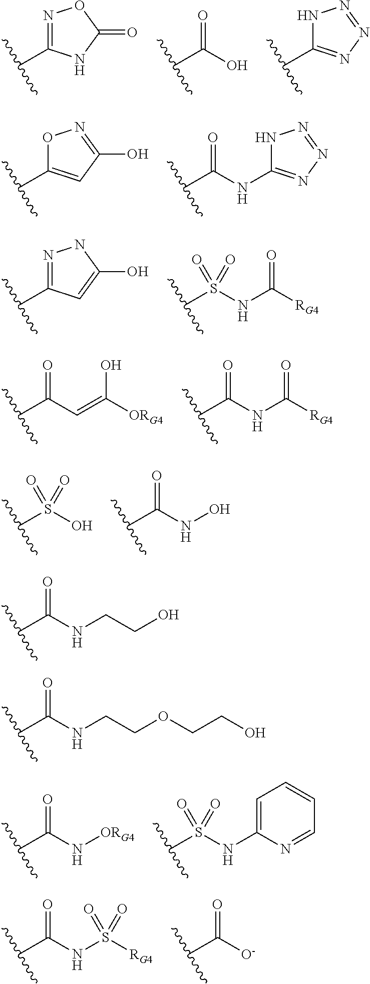

wherein R_{G4} is selected from C₁-C₆alkyl optionally substituted by 1 to 3 halogen atoms, C₂-C₆alkenyl, C₂-C₆alkynyl and C₃-C₆cycloalkyl,
R₄ represents a hydrogen, fluorine, chlorine or bromine atom, a methyl, a hydroxyl or a methoxy group,
R₅ represents a group selected from: C₁-C₆alkyl optionally substituted by 1 to 3 halogen atoms; C₂-C₆alkenyl; C₂-C₆alkynyl; halogen or —CN,
R₆ represents a group selected from:
hydrogen;
a linear or branched —C₁-C₆alkylene-Ra group;
   —C₂-C₆alkenyl;
   λ₂—O—R⁷;

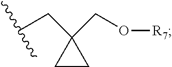

—X₂—NSO₂—R₇;
—C═C(R₉)—Y₁—O—R₇;
C₃-C₆cycloalkyl;
C₃-C₆heterocycloalkyl optionally substituted by a hydroxyl group;
C₃-C₆cycloalkylene-Y₂—R₇;
C₃-C₆heterocycloalkylene-Y₂—R₇ group,
a heteroarylene-R₇ group optionally substituted by a linear or branched C₁-C₆alkyl group,
R₇ represents a group selected from: linear or branched C₁-C₆alkyl group; (C₃-C₆)cycloalkylene-R₈; or:

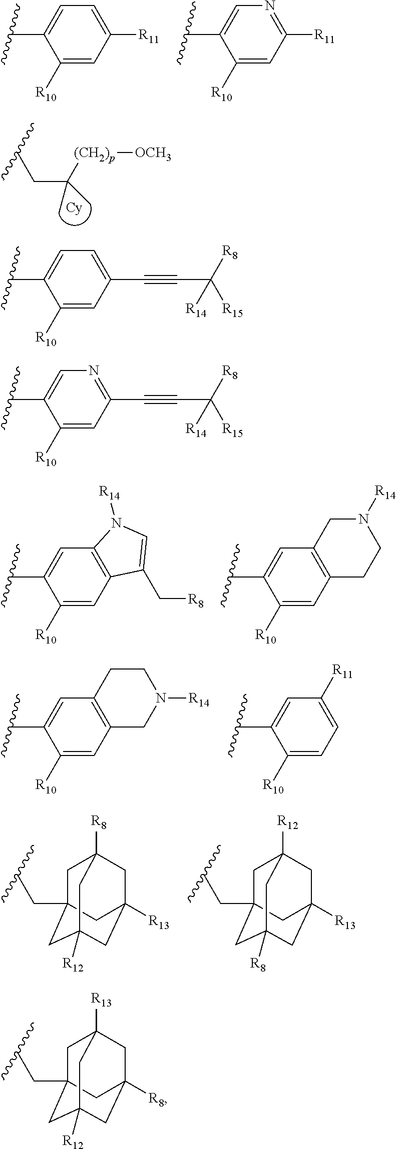

wherein Cy represents a C₃-C₈cycloalkyl,
R₈ represents a group selected from: hydrogen; linear or branched C₁-C₆alkyl, —NR′ₐR′_{b};
   —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}, —X′₂—NR′ₐR′_{b}, —NR′_{c}—X′₂—N₃ and —NR′_{c}—X′₂
R₉ represents a group selected from linear or branched C₁-C₆alkyl, trifluoromethyl, hydroxyl, halogen, C₁-C₆alkoxy,
R₁₀ represents a group selected from hydrogen, fluorine, chlorine, bromine, —CF₃ and methyl,
R₁₁ represents a group selected from hydrogen, C₁-C₆alkylene-R₈, —O—C₁-C₃alkylene-R₈, —CO—NRₕRᵢ and —CH═CH—C₁-C₄alkylene-NRₕRᵢ, —CH═CH—CHO, C₃-C₈cycloalkylene-CH₂—R₈, C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ with the carbon atom carrying them form a cyclohexyl,
Rₕ and Rᵢ, independently of one another, represent a hydrogen or a linear or branched C₁-C₆alkyl group,
X₁ and X₂ independently of one another, represent a linear or branched C₁-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl or C₁-C₆alkoxy groups; C₁-C₆alkylene-SO₂OH; C₁-C₆alkylene-SO₂O—; C₁-C₆alkylene-COOH; C₁-C₆alkylene-PO(OH)₂; C₁-C₆alkylene-NR′_{d}R′ₑ; C₁-C₆alkylene-N⁺R′_{d}R′ₑR′_{f}; C₁-C₆alkylene-O—C₁-C₆alkylene-OH; C₁-C₆alkylene-phenyl wherein the
phenyl may be substituted by a hydroxyl or a C₁-C₆alkoxy group; the group:

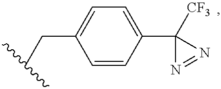 [SMILES: *Cc1ccc(C2(C(F)(F)F)N=N2)cc1]

or R′ₐ and R′_{b} form with the nitrogen atom carrying them a cycle B₃, or R′ₐ, R′_{b} and R′_{c} form with the nitrogen atom carrying them a bridged C₃-C₈ hetero cycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄, or R′_{d}, R′ₑ and R′_{f} form with the nitrogen atom carrying them a bridged C₃-C₈ heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, —NH—SO₂—,
m=0, 1 or 2,
B₁, B₂, B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from: fluorine, bromine, chlorine, linear or branched C₁-C₆alkyl, hydroxyl, —NH₂, oxo or piperidinyl,
   wherein one of the R₃ and R₈ groups, if present, is covalently attached to the linker, and wherein the valency of an atom is not exceeded by virtue of one or more substituents bonded thereto;
   and the remaining variables are as described in the first, second, third, fourth, fifth, sixth, seventh or eighth embodiment.

In an eleventh embodiment of the present disclosure, for the compound of formula (A), or or an enantiomer, a diastereomer, and/or a pharmaceutically acceptable salt thereof in the ninth or tenth embodiment, R₁ is linear or branched C₁-C₆alkyl and R₂ is H; and the remaining variables are as described in the ninth or tenth embodiment.

In a twelfth embodiment of the present disclosure, for the compound of formula (A), or an enantiomer, a diastereomer, and/or a pharmaceutically acceptable salt thereof, D comprises a compound of Formula (II):

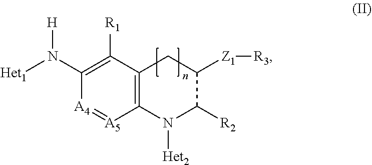

or an enantiomer, a diastereoisomer, and/or a pharmaceutically acceptable salt of any one of the foregoing, wherein:
n=0, 1 or 2,
------ represents a single or a double bond,
A₄ and A₅ independently of one another represent a carbon or a nitrogen atom,
Z₁ represents a bond, —N(R)—, or —O—, wherein R represents a hydrogen or a linear or branched C₁-C₆alkyl,
R₁ represents a group selected from the group consisting of: hydrogen; a linear or branched C₁-C₆alkyl optionally substituted by a hydroxyl or a C₁-C₆alkoxy group; a C₃-C₆cycloalkyl; trifluoromethyl; and a linear or branched C₁-C₆alkylene-heterocycloalkyl wherein the heterocycloalkyl group is optionally substituted by a linear or branched C₁-C₆alkyl group;
R₂ represents a hydrogen or a methyl;
R₃ represents a group selected from the group consisting of: hydrogen; a linear or branched C₁-C₄alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:
   —X₁-≡CH,
Rₐ and R_{b} independently of one another represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl groups; a C₁-C₆alkylene-SO₂OH; a C₁-C₆alkylene-SO₂O—; a C₁-C₆alkylene-COOH; a C₁-C₆alkylene-PO(OH)₂; a C₁-C₆alkylene-NR_{d}Rₑ; a C₁-C₆alkylene-N⁺R_{d}RₑR_{f}; a C₁-C₆alkylene-phenyl wherein the phenyl may be substituted by a C₁-C₆alkoxy group; and the group:

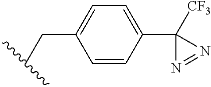 [SMILES: *Cc1ccc(C2(C(F)(F)F)N=N2)cc1]

or Rₐ and R_{b} form with the nitrogen atom carrying them a cycle B₁;
or Rₐ, R_{b} and R_{c} form with the nitrogen atom carrying them a bridged C₃-C₈ heterocycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a cycle B₂,
or R_{d}, Rₑ and R_{f} form with the nitrogen atom carrying them a bridged C₃-C₈ heterocycloalkyl,
Het₁ represents a group selected from the group consisting of:

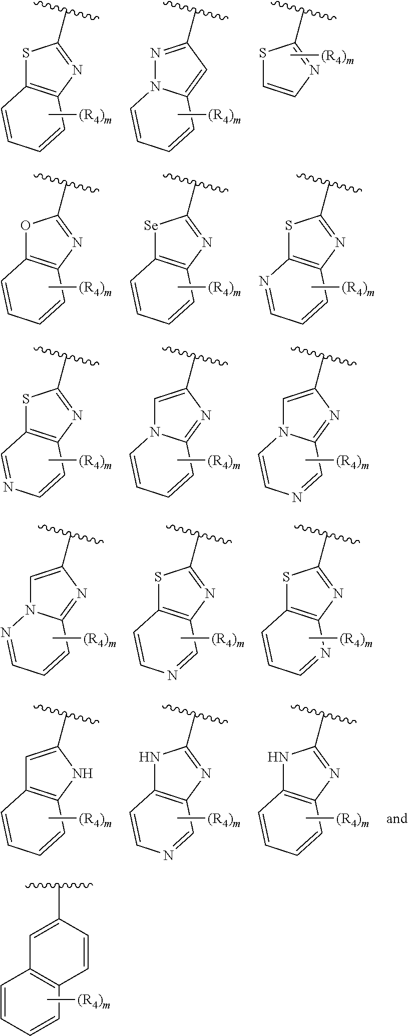

Het₂ represents a group selected from the group consisting of:

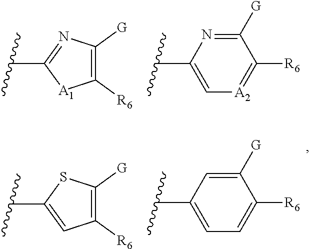

A₁ is —NH—, —N(C₁-C₃alkyl), O, S or Se,
A₂ is N, CH or C(R₅),
G is selected from the group consisting of:
   —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, —C₁-C₆alkyl optionally substituted by a hydroxyl group, a halogen, —NO₂, and —CN, in which:
R_{G1} and R_{G2} at each occurrence are each independently selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂—C alkynyl, a C₃-C₆cycloalkyl, phenyl and —(CH₂)₁₋₄-phenyl;
R_{G3} is selected from the group consisting of a C₁-C₆alkyl optionally substituted by 1 to 3 halogen atoms, a C₂-C₆alkenyl, a C₂-C₆alkynyl, a C₃-C₆cycloalkyl, phenyl and —(CH₂)₁₋₄-phenyl; or R_{G1} and R_{G2}, together with the atom to which each is attached are combined to form a C₃-C₈heterocycloalkyl; or in the alternative, G is selected from the group consisting of:

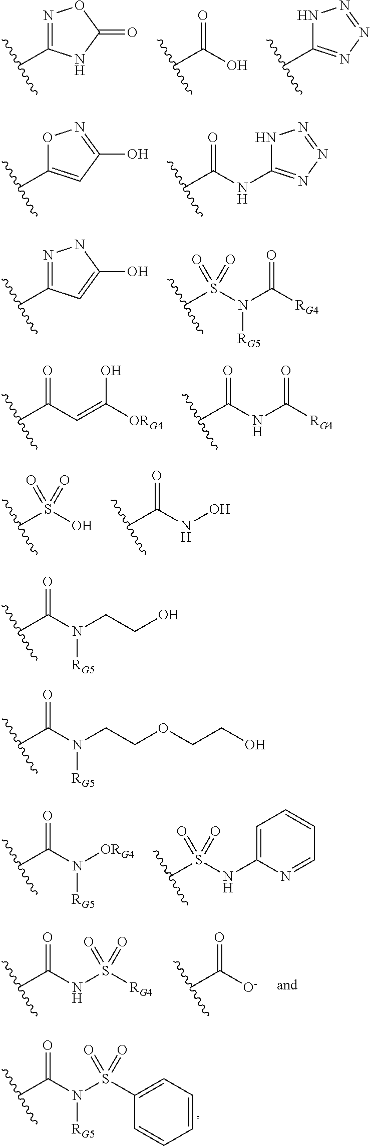

wherein R_{G4} is selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆ alkenyl, a C₂-C₆alkynyl and a C₃-C₆cycloalkyl, and R_{G5} represents a hydrogen atom or a C₁-C₆alkyl group optionally substituted by 1 to 3 halogen atoms,
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a C₂-C₆alkenyl; a C₂-C₆alkynyl; a halogen and —CN,
R₆ represents a group selected from the group consisting of: hydrogen;
a linear or branched —C₁-C₆alkylene-Rₐ group;
a —C₂-C₆alkenyl;
   —X₂—O—R⁷;

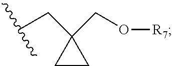

—X₂—NSO₂—R₇;
—C═C(R₉)—Y₁—O—R₇;
a C₃-C₆cycloalkyl;
a C₃-C₆heterocycloalkyl optionally substituted by a hydroxyl group;
a C₃-C₆cycloalkylene-Y₂—R₇;
a C₃-C₆heterocycloalkylene-Y₂—R₇ group, and
a heteroarylene-R₇ group optionally substituted by a linear or branched C₁-C₆alkyl group,
R₇ represents a group selected from the group consisting of: a linear or branched C₁-C₆alkyl group; a (C₃-C₆)cycloalkylene-R₈;

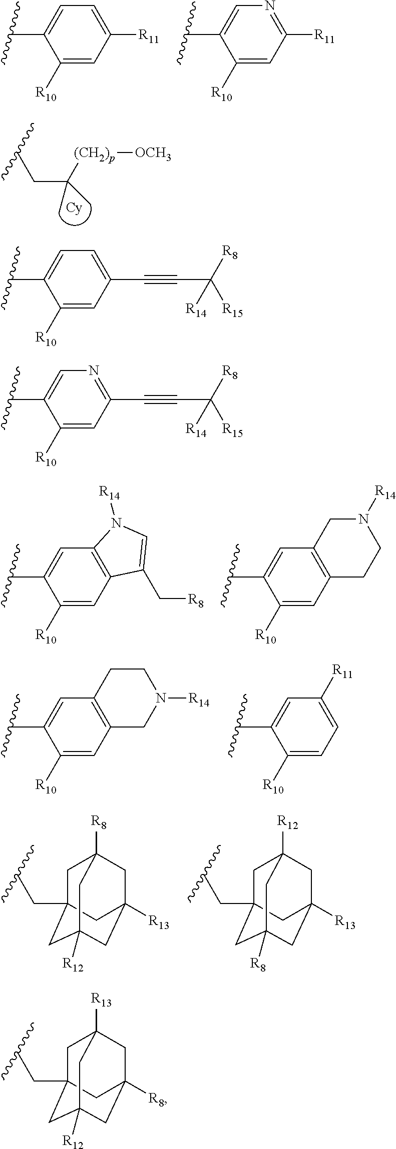

wherein Cy represents a C₃-C₈cycloalkyl,
R₈ represents a group selected from the group consisting of: hydrogen; a linear or branched C₁-C₆alkyl, —NR′ₐR′_{b}; —NR′ₐ CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}, —X′₂—NR′ₐR′_{b}, —NR′_{c}—X′₂—N₃ and
   —NR′_{c}—X′₂-≡CH,
R₉ represents a group selected from the group consisting of a linear or branched C₁-C₆alkyl, trifluoromethyl, hydroxyl, a halogen, and a C₁-C₆alkoxy,
R₁₀ represents a group selected from the group consisting of hydrogen, fluorine, chlorine, bromine, —CF₃ and methyl,
R₁₁ represents a group selected from the group consisting of hydrogen, a halogen, a C₁-C₃alkylene-R₈, a —O—C₁-C₃alkylene-R₈, —CO—NRₕRᵢ and a —CH═CH—C₁-C₄alkylene-NRₕRᵢ, —CH═CH—CHO, a C₃-C₈cycloalkylene-CH₂—R₈, and a C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ represents a linear or branched C₁-C₄alkylene group optionally substituted by one or two groups selected from the group consisting of trifluoromethyl, hydroxyl, a halogen, and a C₁-C₆alkoxy,
X₂ represents a linear or branched C₁-C₆alkylene group optionally substituted by one or two groups selected from the group consisting of trifluoromethyl, hydroxyl, a halogen, and a C₁-C₆alkoxy,
X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O—; a C₁-C₆alkylene-COOH; a C₁-C₆alkylene-PO(OH)₂; a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy group; and the group:

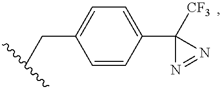 [SMILES: *Cc1ccc(C2(C(F)(F)F)N=N2)cc1]

or R′ₐ and R′_{b} form with the nitrogen atom carrying them a cycle B₃,
or R′ₐ, R′_{b} and R′_{c} form with the nitrogen atom carrying them a bridged C₃-C₈ heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈ heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, or —NH—SO₂—,
m=0, 1 or 2,
B₁, B₂, B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl,
   wherein one of the R₃, R₈ and G groups, if present, is covalently attached to the linker, and wherein the valency of an atom is not exceeded by virtue of one or more substituents bonded thereto; and the remaining variables are as described in the first, second, third, fourth, fifth, sixth, seventh or eighth embodiment.

In a thirteenth embodiment of the present disclosure, for the compound of formula (A), or an enantiomer, a diastereomer, and/or a pharmaceutically acceptable salt thereof, D in Formula (A) comprises a compound of Formula (II):

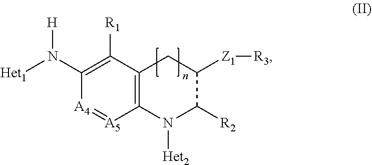

wherein:
n=0, 1 or 2,
------ represents a single or a double bond.
A₄ and A₅ independently of one another represent a carbon or a nitrogen atom,
Z₁ represents a bond, —N(R)—, or —O—, wherein R represents a hydrogen or a linear or branched C₁-C₆alkyl,
R₁ represents a group selected from: hydrogen; linear or branched C₁-C₆alkyl optionally substituted by a hydroxyl or a C₁-C₆alkoxy group; C₃-C₆cycloalkyl; trifluoromethyl; linear or branched C₁-C₆alkylene-heterocycloalkyl wherein the heterocycloalkyl group is optionally substituted by a a linear or branched C₁-C₆alkyl group;
R₂ represents a hydrogen or a methyl;
R₃ represents a group selected from: hydrogen; linear or branched C₁-C₄alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:
   —X₁-≡CH,
Rₐ and R_{b} independently of one another represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl groups; C₁-C₆alkylene-SO₂OH; C₁-C₆alkylene-SO₂O—; C₁-C₆alkylene-COOH; C₁-C₆alkylene-PO(OH)₂; C₁-C₆alkylene-NR_{d}Rₑ; C₁-C₆alkylene-N⁺R_{d}RₑR_{f}; C₁-C₆alkylene-phenyl wherein the phenyl may be substituted by a C₁-C₆alkoxy group; the group:

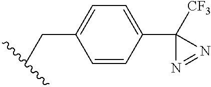 [SMILES: *Cc1ccc(C2(C(F)(F)F)N=N2)cc1]

or Rₐ and R_{b} form with the nitrogen atom carrying them a cycle B₁; or Rₐ, R_{b} and R_{c} form with the nitrogen atom carrying them a bridged C₃-C₈ heterocycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a a cycle B₂, or R_{d}, Rₑ and R_{f} form with the nitrogen atom carrying them a bridged C₃-C₈ heterocycloalkyl,
Het₁ represents a group selected from:

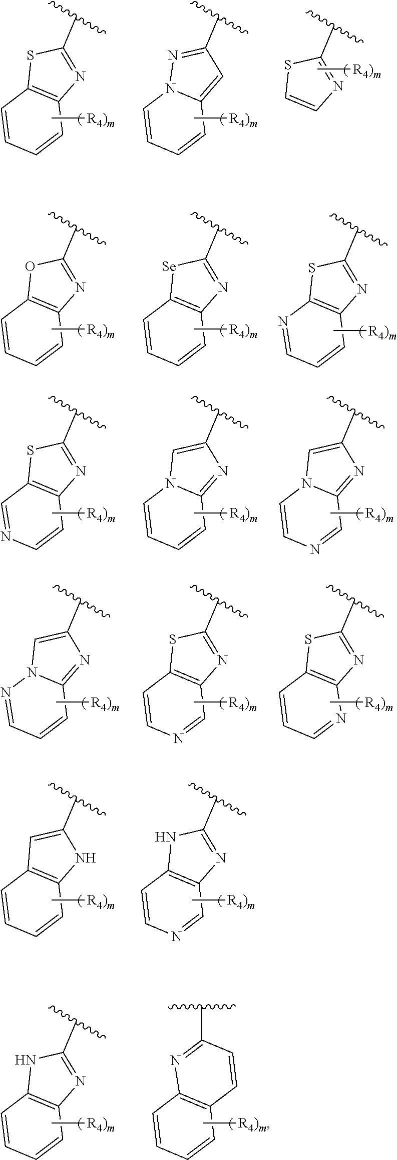

Het₂ represents a group selected from:

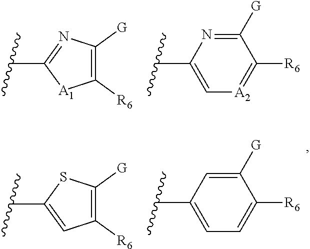

A₁ is —NH—, —N(C₁-C₃alkyl), O, S or Se,
A₂ is N, CH or C(R₅),
G is selected from the group consisting of:
   —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, halogen, —NO₂, and —CN, in which:
R_{G1} and R_{G2} at each occurrence are each independently selected from the group consisting of hydrogen, C₁-C₆alkyl optionally substituted by 1 to 3 halogen atoms, C₂-C₆alkenyl, C₂—C alkynyl, C₃-C₆cycloalkyl, phenyl and —(CH₂)₁₋₄-phenyl;
R_{G3} is selected from the group consisting of C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl; or R_{G1} and R_{G2}, together with the atom to which each is attached are combined to form a C₃-C₃heterocycloalkyl; or in the alternative, G is selected from the group consisting of:

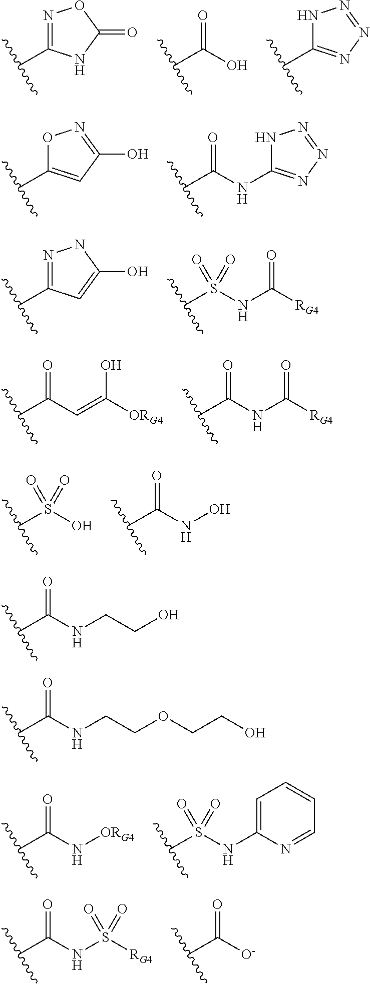

wherein R_{G4} is selected from C₁-C₆alkyl optionally substituted by 1 to 3 halogen atoms, C₂-C₆ alkenyl, C₂-C₆alkynyl and C₃-C₆cycloalkyl,
R₄ represents a hydrogen, fluorine, chlorine or bromine atom, a methyl, a hydroxyl or a methoxy group,
R₅ represents a group selected from: C₁-C₆alkyl optionally substituted by 1 to 3 halogen atoms; C₂-C₆alkenyl; C₂-C₆alkynyl; halogen or —CN,
Rₑ represents a group selected from: hydrogen;
a linear or branched —C₁-C₆alkylene-Rₐ group;
   —C₂-C₆alkenyl;
   —X₂—O—R⁷;

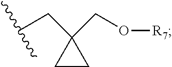

—X₂—NSO₂—R₇;
C═C(R₉)—Y₁—O—R₇;
C₃-C₆cycloalkyl;
C₃-C₆heterocycloalkyl optionally substituted by a hydroxyl group;
C₃-C₆cycloalkylene-Y₂—R₇;
C₃-C₆heterocycloalkylene-Y₂—R₇ group,
a heteroarylene-R₇ group optionally substituted by a linear or branched C₁-C₆alkyl group,
R₇ represents a group selected from: linear or branched C₁-C₆alkyl group; (C₃-C₆)cycloalkylene-R₈; or:

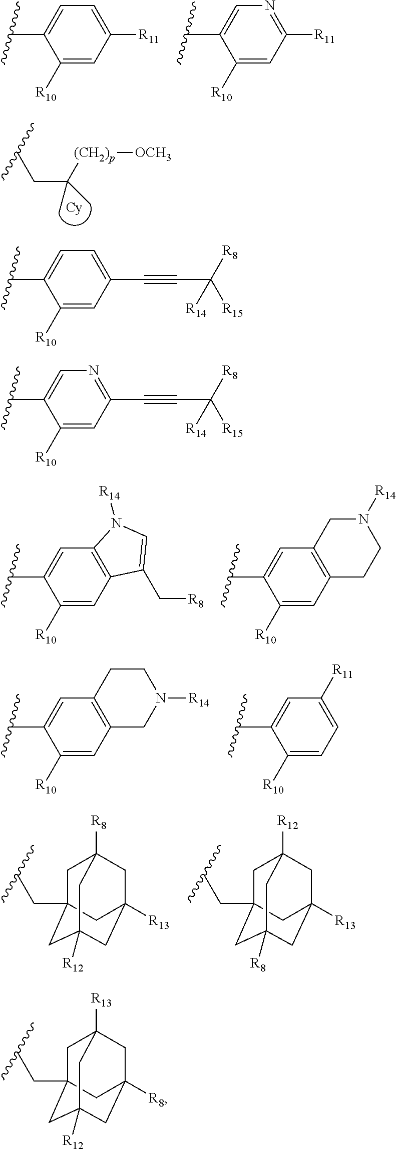

wherein Cy represents a C₃-C₈cycloalkyl,
R₈ represents a group selected from: hydrogen; linear or branched C₁-C₆alkyl, —NR′ₐR′_{b}; —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}, —X′₂—NR′ₐR′_{b}, —NR′_{c}—X′₂—N₃ and: —NR′_{c}—X′₂-≡CH,
R₉ represents a group selected from linear or branched C₁-C₆alkyl, trifluoromethyl, hydroxyl, halogen, C₁-C₆alkoxy,
R₁₀ represents a group selected from hydrogen, fluorine, chlorine, bromine, —CF₃ and methyl,
R₁₁ represents a group selected from hydrogen, halogen, C₁-C₃alkylene-R₈, —O—C₁-C₃alkylene-R₈, —CO—NRₕRᵢ and —CH═CH—C₁-C₄alkylene-NRₕRᵢ, —CH═CH—CHO, C₃-C₈cycloalkylene-CH₂-R₈, C₃-C₈heterocycloalkylene-CH₂-R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ with the carbon atom carrying them form a cyclohexyl,
Rₕ and Rᵢ, independently of one another, represent a hydrogen or a linear or branched C₁-C₆alkyl group,
X₁ represents a linear or branched
C₁-C₄alkylene group optionally substituted by one or two groups selected from trifluoromethyl, hydroxyl, halogen, C₁-C₆alkoxy,
X₂ represents a linear or branched
C₁-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl or C₁-C₆alkoxy groups; C₁-C₆alkylene-SO₂OH; C₁-C₆alkylene-SO₂O⁻;
   C₁-C₆alkylene-COOH; C₁-C₆alkylene-PO(OH)₂; C₁-C₆alkylene-NR′_{d}R′ₑ;
C₁-C₆alkylene-N⁺R′_{d}R′ₑR′_{f}; C₁-C₆alkylene-O—C₁-C₆alkylene-OH; C₁-C₆alkylene-phenyl wherein the phenyl may be substituted by a hydroxyl or a C₁-C₆alkoxy group; the group:

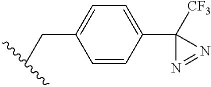 [SMILES: *Cc1ccc(C2(C(F)(F)F)N=N2)cc1]

or R′ₐ and R′_{b} form with the nitrogen atom carrying them a cycle B₃, or R′ₐ, R′_{b} and R′_{c} form with the nitrogen atom carrying them a bridged C₃-C₈ heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈ heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, —NH—SO₂—,
+m=0, 1 or 2,
B₁, B₂, B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from: fluorine, bromine, chlorine, linear or branched C₁-C₆alkyl, hydroxyl, —NH₂, oxo or piperidinyl,
   wherein one of the R₃ and R₈ groups, if present, is covalently attached to the linker, and wherein the valency of an atom is not exceeded by virtue of one or more substituents bonded thereto;
   and the remaining variables are as described in the first, second, third, fourth or fifth embodiment.

In a fourteenth embodiment of the present disclosure, for the compound of formula (A), or an enantiomer, a diastereomer, and/or a pharmaceutically acceptable salt thereof in the embodiment, A₄ and A₅ both represent a nitrogen atom, R₁ is linear or branched C₁₋₆alkyl; R₂ is H; n is 1; and ----- represents a single bond; and the remaining variables are as described in the twelfth or thirteenth embodiment.

In a fifteenth embodiment of the present disclosure, for the compound of formula (A), or an enantiomer, a diastereomer, and/or a pharmaceutically acceptable salt thereof, D comprises a compound of formula (IA) or (IIA):

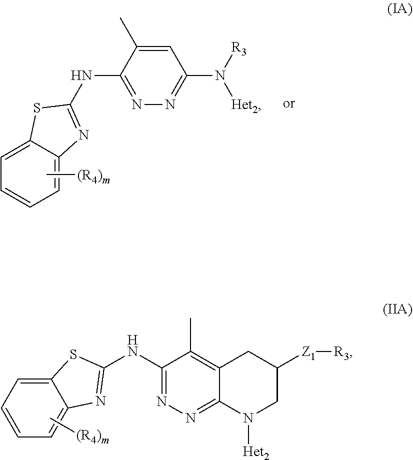

or an enantiomer, a diastereoisomer, and/or a pharmaceutically acceptable salt of any one of the foregoing, wherein:
Z₁ represents a bond or —O—,
R₃ represents a group selected from the group consisting of: hydrogen; a C₃-C₆cycloalkyl; a linear or branched C₁-C₆alkyl; —X₁—NRₐR_{b}; —X₁—N⁺RₐR_{b}R_{c}; —X₁—O—R_{c}; —X₁—N₃ and —X₁-≡CH,
Rₐ and R_{b} independently of one another represent a group selected from the group consisting of: hydrogen; a linear or branched C₁-C₆alkyl optionally substituted by one or two hydroxyl groups; and a C₁-C₆alkylene-SO₂O—,
R_{c} represents a hydrogen or a linear or branched C₁-C₆alkyl group,
Het₂ represents a group selected from the group consisting of:

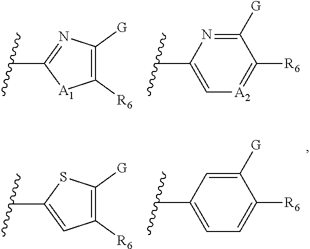

A₁ is —NH—, —N(C₁-C₆alkyl), O, S or Se,
A₂ is N, CH or C(R₅),
G is selected from the group consisting of:
   —C(O)OH, —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, —C₁-C₆alkyl optionally substituted by a hydroxyl group, —C(O)NR_{G5}S(O)₂R_{G4}, halogen, —NO₂, and —CN, in which:
R_{G1}, R_{G2}, R_{G4} and R_{G5} at each occurrence are each independently selected from the group consisting of hydrogen, and a C₁-C₆alkyl optionally substituted by 1 to 3 halogen atoms;
R_{G3} is a C₁-C₆alkyl optionally substituted by 1 to 3 halogen atoms; or
   R_{G1} and R_{G2}, together with the atom to which each is attached are combined to form a C₃-C₈heterocycloalkyl;
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a halogen and —CN,
R₆ represents a group selected from the group consisting of:
a linear or branched —C₁-C₆alkylene-R₈ group;
—X₂—O—R₇; and
a heteroarylene-R₇ group optionally substituted by a linear or branched C₁-C₆alkyl group,
R₇ represents a group selected from the group consisting of: a linear or branched C₁-C₆alkyl group; (C₃-C₆)cycloalkylene-R₈;

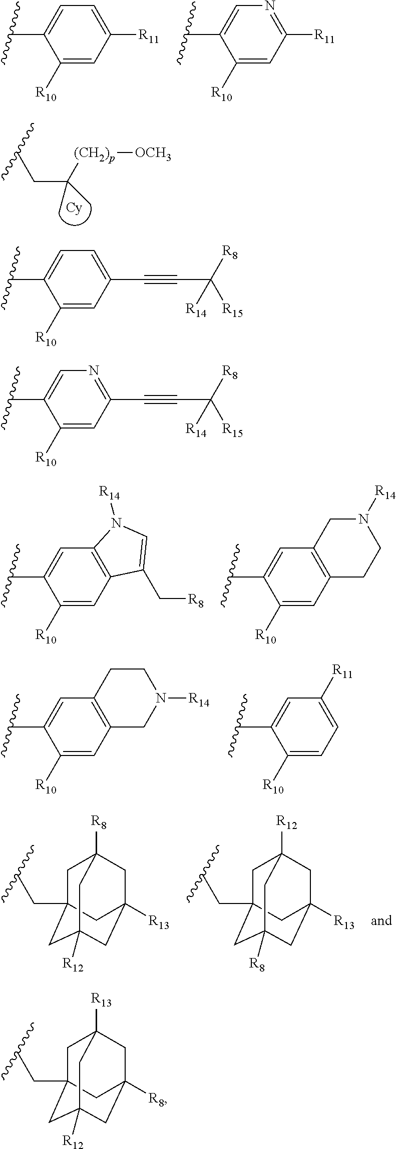

wherein Cy represents a C₃-C₈cycloalkyl,
R₈ represents a group selected from the group consisting of: hydrogen; a linear or branched C₁-C₆alkyl, —NR′ₐR′_{b}; —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}; —X′₂—NR′ₐR′_{b}; —NR′_{c}—X′₂—N₃ and
   —NR′_{c}—X′₂-≡CH,
R₁₀ represents a group selected from the group consisting of hydrogen, fluorine, chlorine, bromine, —CF₃ and methyl,
R₁₁ represents a group selected from the group consisting of hydrogen, a C₁-C₃alkylene-R₈, —O—C₁-C₆alkylene-R₈, —CO—NRₕRᵢ, —CH═CH—C₁-C₄alkylene-NRₕRᵢ, —CH═CH—CHO, a C₃-C₈cycloalkylene-CH₂—R₈, and a C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a a cyclohexyl,
Rₕ and Rᵢ, independently of one another, represent a hydrogen or a linear or branched C₁-C₆alkyl group,
X₁ and X₂ independently of one another, represent a linear or branched C₁-C₆alkylene group optionally substituted by one or two groups selected from the group consisting of trifluoromethyl, hydroxyl, a halogen, and C₁-C₆alkoxy,
X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O—; a C₁-C₆alkylene-COOH; a C₁-C₆alkylene-PO(OH)₂; a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy group; and the group:

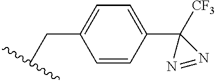 [SMILES: *Cc1ccc(C2(C(F)(F)F)N=N2)cc1]

or R′ₐ and R′_{b} form with the nitrogen atom carrying them a cycle B₃,
or R′ₐ, R′_{b} and R′_{c} form with the nitrogen atom carrying them a bridged C₃-C₆heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₆heterocycloalkyl,
m=0, 1 or 2,
p=1, 2, 3 or 4,
   B₃ and B₄, independently of one another, represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl;
and the remaining variables are as described in the first, second, third, fourth, fifth, sixth, seventh, eighth, ninth, tenth, eleventh, twelfth, thirteenth or fourteenth embodiment.

In a sixteenth embodiment of the present disclosure, for the compound of formula (A), or an enantiomer, a diastereomer, and/or a pharmaceutically acceptable salt thereof, D comprises a compound of formula (IA) or (IIA):

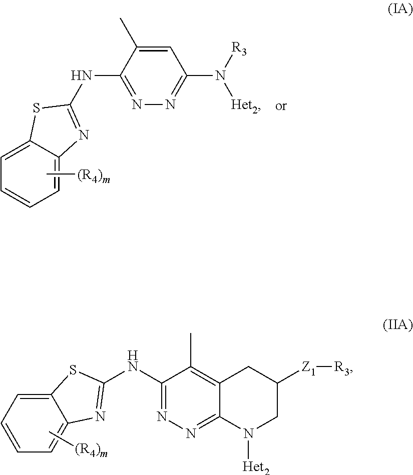

wherein:
Z₁ represents a bond or —O—,
R₃ represents a group selected from: hydrogen; C₃-C₆cycloalkyl; linear or branched C₁-C₆alkyl; —X₁—NRₐR_{b}; —X₁—N⁺RₐR_{b}R_{c}; —X₁—O—R_{c}; —X₁—N₃ and —X₁-≡CH,
Rₐ and R_{b} independently of one another represent a group selected from: hydrogen; linear or branched C₁-C₆alkyl optionally substituted by one or two hydroxyl groups; and C₁-C₆alkylene-SO₂O⁻,
R_{c} represents a hydrogen or a linear or branched C₁-C₆alkyl group,
Het₂ represents a group selected from:

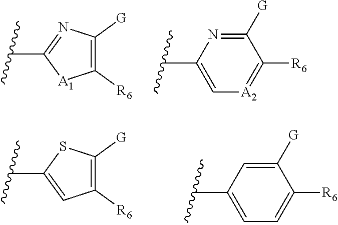

A₁ is —NH—, —N(C₁-C₆alkyl), O, S or Se,
A₂ is N, CH or C(R₅),
G is selected from the group consisting of:
—C(O)OH, —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, halogen, —NO₂, and —CN, in which:
R_{G1} and R_{G2} at each occurrence are each independently selected from the group consisting of hydrogen, and C₁-C₆alkyl optionally substituted by 1 to 3 halogen atoms;
R_{G3} is C₁-C₆alkyl optionally substituted by 1 to 3 halogen atoms; or
   R_{G1} and R_{G2}, together with the atom to which each is attached are combined to form a C₃-C₆heterocycloalkyl;
R₄ represents a hydrogen, fluorine, chlorine or bromine atom, a methyl, a hydroxyl or a methoxy group,
R₅ represents a group selected from: C₁-C₆alkyl optionally substituted by 1 to 3 halogen atoms; halogen or —CN,
R₆ represents a group selected from:
a linear or branched —C₁-C₆alkylene-R₈ group;
—X₂—O—R₇; and
a heteroarylene-R₇ group optionally substituted by a linear or branched C₁-C₆alkyl group,
R₇ represents a group selected from: linear or branched C₁-C₆alkyl group; (C₃-C₆)cycloalkylene-R₈; or:

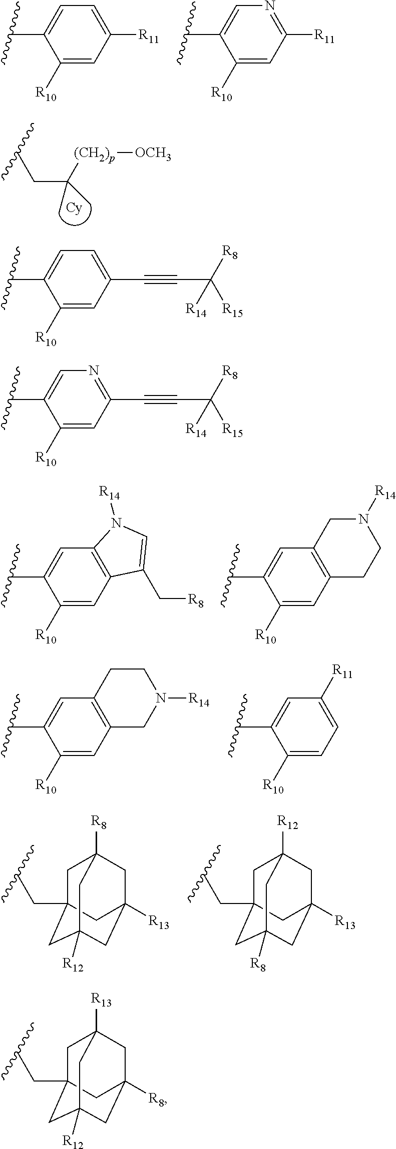

wherein Cy represents a C₃-C₈cycloalkyl,
R₈ represents a group selected from: hydrogen; linear or branched C₁-C₆alkyl, —NR′ₐR′_{b};
   —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}; —X′₂—NR′ₐR′_{b}: —NR′_{c}—X′₂—N₃ and:
   —NR′_{c}—X′₂-≡CH,
R₁₀ represents a group selected from hydrogen, fluorine, chlorine, bromine, —CF₃ and methyl,
R₁₁ represents a group selected from hydrogen, C₁-C₆alkylene-R₈, —O—C₁-C₃alkylene-R₈, —CO—NRₕRᵢ and —CH═CH—C₁-C₄alkylene-NRₕRᵢ, —CH═CH—CHO, C₃-C₈cycloalkylene-CH₂—R₈, C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a a cyclohexyl,
Rₕ and Rᵢ, independently of one another, represent a hydrogen or a linear or branched C₁-C₆alkyl group,
X₁ and X₂ independently of one another, represent a linear or branched C₀-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl or C₁-C₆alkoxy groups; C₁-C₆alkylene-SO₂OH; C₁-C₆alkylene-SO₂O—; C₁-C₆alkylene-COOH; C₁-C₆alkylene-PO(OH)₂; C₁-C₆alkylene-NR′_{d}R′ₑ; C₁-C₆alkylene-N⁺R′_{d}R′ₑR′_{f}; C₁-C₆alkylene-O—C₁-C₆alkylene-OH; C₁-C₆alkylene-phenyl wherein the phenyl may be substituted by a hydroxyl or a C₁-C₆alkoxy group;
the group:

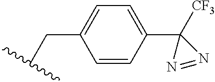 [SMILES: *Cc1ccc(C2(C(F)(F)F)N=N2)cc1]

or R′ₐ and R′_{b} form with the nitrogen atom carrying them a cycle B₃,
or R′ₐ, R′_{b} and R′_{c} form with the nitrogen atom carrying them a bridged C₃-C₆heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₆heterocycloalkyl,
m=0, 1 or 2,
B₃ and B₄, independently of one another, represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from: fluorine, bromine, chlorine, linear or branched C₁-C₆alkyl, hydroxyl, —NH₂, oxo or piperidinyl.; and the remaining variables are as described in the first, second, third, fourth, fifth, sixth, seventh, eighth, ninth, tenth, eleventh, twelfth, thirteenth or fourteenth embodiment.

In a seventeenth embodiment of the present disclosure, for the compound of formula (A), or an enantiomer, a diastereomer, and/or a pharmaceutically acceptable salt thereof, R₇ represents a group selected from: linear or branched C₁-C₆alkyl group; (C₃-C₆)cycloalkylene-R₈; or:

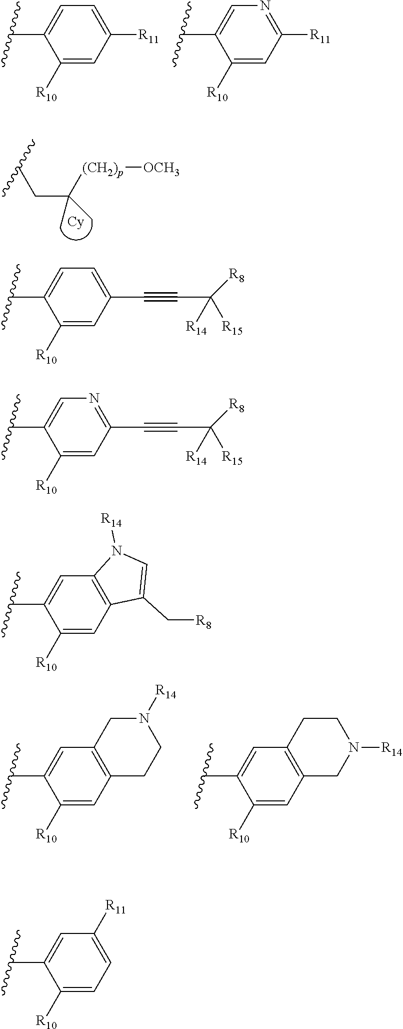

wherein:
Cy represents a C₃-C₆cycloalkyl; and
   the remaining variables are as described in the first, second, third, fourth, fifth, sixth, seventh, eighth, ninth, tenth, eleventh, twelfth, thirteenth, fourteenth, fifteenth or sixteenth embodiment.

In an eighteenth embodiment of the present disclosure, for the compound of formula (A), or an enantiomer, a diastereomer, and/or a pharmaceutically acceptable salt thereof, R₇ represents a group selected from:

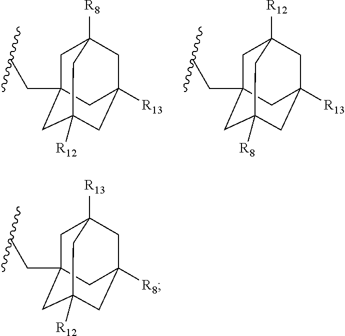

and the remaining variables are as described in the first, second, third, fourth, fifth, sixth, seventh, eighth, ninth, tenth, eleventh, twelfth, thirteenth, fourteenth, fifteenth, sixteenth or seventeenth embodiment.

In a nineteenth embodiment of the present disclosure, for the compound of formula (A), or an enantiomer, a diastereomer, and/or a pharmaceutically acceptable salt thereof, D comprises a compound of formula (IB), (IC-1), (IIB) or (IIC-1):

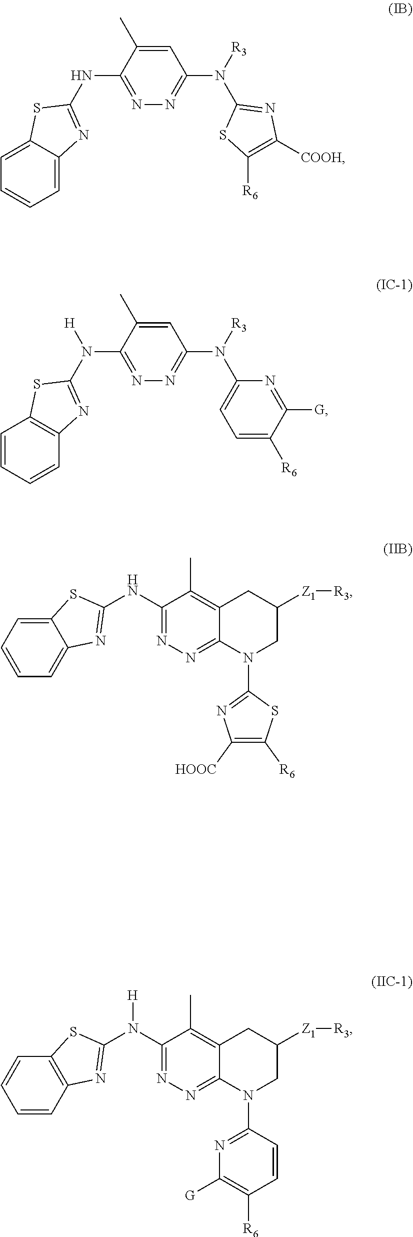

or an enantiomer, a diastereoisomer, and/or a pharmaceutically acceptable salt of any one of the foregoing, wherein:
for formula (IB) or (IC-1), R₃ represents a group selected from the group consisting of: hydrogen; a linear or branched C₁-C₆alkyl; —X₁—NRₐR_{b}; —X₁—N⁺RₐR_{b}R_{c}; —X₁—O—R_{c}; —X₁—N₃ and —X₁-≡CH,
for formula (IIB) or (IIC-1), Z₁ represents a bond, and R₃ represents hydrogen; or Z, represents —O—, and R₃ represents —X₁—NRₐR_{b},
for formula (IC-1), G is selected from the group consisting of —C(O)OH and —C(O)NR_{G1}R_{G2};
for formula (IIC-1), G is selected from the group consisting of —C(O)OH, —C(O)NR_{G1}R_{G2}, —C(O)R_{G2}, —C₁-C₆alkyl optionally substituted by a hydroxyl group and —C(O)NR_{G5}S(O)₂R_{G4}, in which R_{G1}, R_{G2}, R_{G4} and R_{G5} at each occurrence are each independently selected from the group consisting of hydrogen and a C₁-C₆alkyl optionally substituted by 1 to 3 halogen atoms;
Rₐ and R_{b} independently of one another represent a group selected from the group consisting of: hydrogen; a linear or branched C₁-C₆alkyl optionally substituted by one or two hydroxyl groups; and a C₁-C₆alkylene-SO₂O—,
R_{c} represents a hydrogen or a linear or branched C₁-C₆alkyl group
R₆ represents a linear or branched —C₁-C₆alkylene-Rₐ group, —X₂—O—R₇, or a heteroarylene-R₇ group optionally substituted by a linear or branched C₁-C₆alkyl group,
R₇ represents a group selected from the group consisting of:

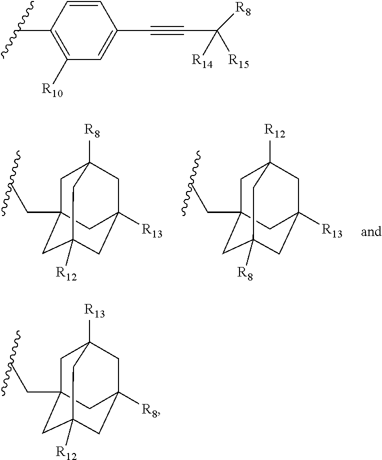

R₈ represents a group selected from the group consisting of: —NR′ₐR′_{b}; —O—X′₂—NR′ₐR′_{b}; and —X′₂—NR′ₐR′_{b},
R₁₀ represents fluorine,
R₁₂ and R₁₃, independently of one another, represent a hydrogen atom or a methyl group,
R₁₄ and R₁₅, independently of one another, represent a hydrogen or a methyl group,
X₁ and X₂ independently of one another, represent a linear or branched C₁-C₆alkylene group optionally substituted by one or two groups selected from the group consisting of trifluoromethyl, hydroxyl, a halogen, and a C₁-C₆alkoxy,
X′₂ represents a linear or branched C₁-C₆alkylene,
R′ₐ and R′_{b} independently of one another, represent a group selected from the group consisting of: hydrogen; a linear or branched C₁-C₆alkyl optionally substituted by one or two hydroxyl or C₁-C₆alkoxy groups; and a C₁-C₆alkylene-NR′_{d}R′ₑ;
or R′ₐ and R′_{b} form with the nitrogen atom carrying them a cycle B₃,
R′_{d}, R′ₑ independently of one another, represents a hydrogen or a linear or branched C₁-C₆alkyl group,
B₃ represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen and nitrogen, (iii) be substituted by one or two groups selected from the group consisting of: fluorine, bromine, chlorine, a linear or branched C₁-C₆alkyl, hydroxyl, and oxo.

In a twentieth embodiment of the present disclosure, for the compound of formula (A), or an enantiomer, a diastereomer, and/or a pharmaceutically acceptable salt thereof, D in Formula (A) comprises a compound of formula (IB), (IC), (IIB) or (IIC):

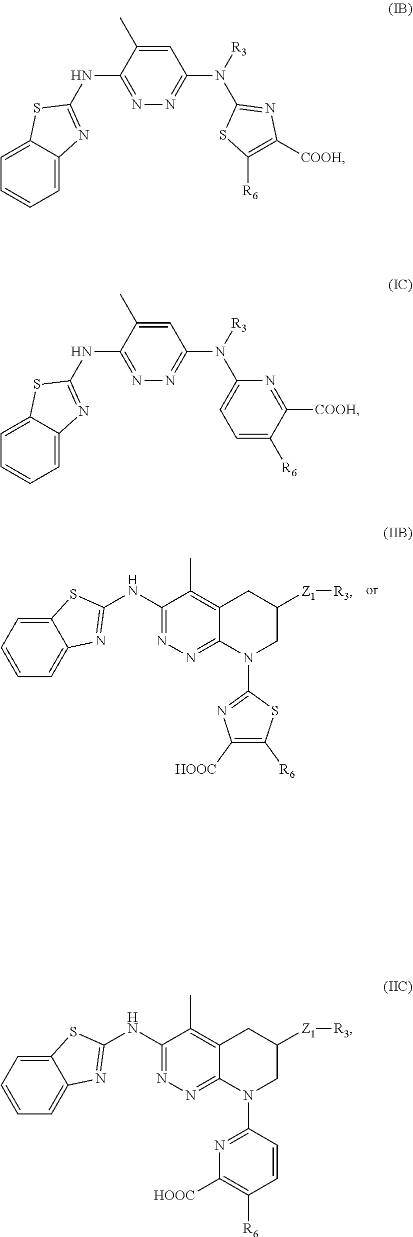

wherein:
for formula (IB) or (IC), R₃ represents a group selected from: hydrogen; linear or branched C₁-C₆alkyl; —X₁—NRₐR_{b}; —X₁—N⁺RₐR_{b}R_{c}; —X₁—O—R₁; —X₁—N₃ and
   —X₁-≡CH,
for formula (IIB) or (IIC), Z₁ represents a bond, and R₃ represents hydrogen; or Z₁ represents —O—, and R₃ represents —X₁—NRₐR_{b},
Rₐ and R_{b} independently of one another represent a group selected from: hydrogen; linear or branched C₁-C₆alkyl optionally substituted by one or two hydroxyl groups; and C₁-C₆alkylene-SO₂O—,
R_{c} represents a hydrogen or a linear or branched C₁-C₆alkyl group
R₆ represents a linear or branched —C₁-C₆alkylene-Rₐ group, —X₂—O—R₇, or a heteroarylene-R₇ group optionally substituted by a linear or branched C₁-C₆alkyl group,
R₇ represents a group selected from:

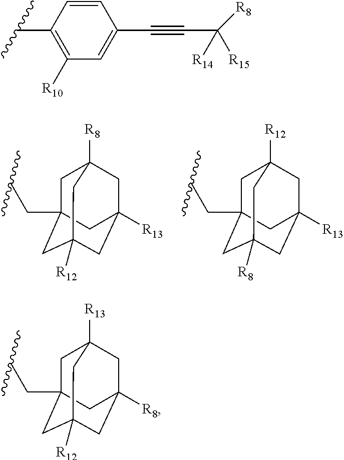

R₈ represents a group selected from: —NR′ₐR′_{b}; —O—X′₂—NR′ₐR′_{b}; and —X′₂—NR′ₐR′_{b},
R₁₀ represents fluorine,
R₁₂ and R₁₃, independently of one another, represent a hydrogen atom or a methyl group,
R₁₄ and R₁₅, independently of one another, represent a hydrogen or a methyl group,
X₁ and X₂ independently of one another, represent a linear or branched C₁-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; linear or branched C₁-C₆alkyl optionally substituted by one or two hydroxyl or C₁-C₆alkoxy groups; C₁-C₆alkylene-NR′_{d}R′ₑ;
or R′ₐ and R′_{b} form with the nitrogen atom carrying them a cycle B₃,
R′_{d}, R′ₑ independently of one another, represents a hydrogen or a linear or branched C₁-C₆alkyl group,
   B₃ represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen and nitrogen, (iii) be substituted by one or two groups selected from: fluorine, bromine, chlorine, linear or branched C₁-C₆alkyl, hydroxyl, and oxo;
and the remaining variables are as described in the first, second, third, fourth, fifth, sixth, seventh, eighth, ninth, tenth, eleventh, twelfth, thirteenth, fourteenth, fifteenth, sixteenth, seventeenth or eighteenth embodiment.

In a twenty-first embodiment of the present disclosure, for the compound of formula (A), or an enantiomer, a diastereomer, and/or a pharmaceutically acceptable salt thereof, R₆ represents —X₂—O—R₇, and R₇ represents the following group:

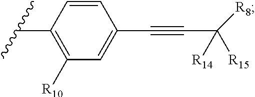

and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth or twentieth embodiment.

In a twenty-second embodiment of the present disclosure, for the compound of formula (A), or or an enantiomer, a diastereomer, and/or a pharmaceutically acceptable salt thereof, R₆ represents a heteroarylene-R₇ group optionally substituted by a linear or branched C₁-C₆alkyl group, and R₇ represents a group selected from:

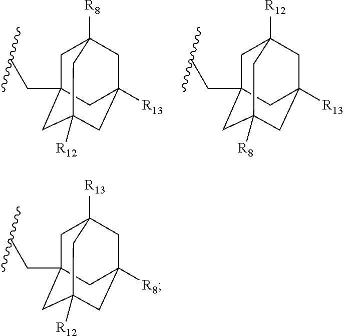

and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth or twentieth embodiment.

In a twenty-third embodiment of the present disclosure, for the compound of formula (A), or an enantiomer, a diastereomer, and/or a pharmaceutically acceptable salt thereof, B₃ represents a C₃-C₆heterocycloalkyl group selected from a pyrrolidinyl group, a piperidinyl group, a piperazinyl group, a morpholinyl group, an azepanyl group, and a 4,4-difluoropiperidin-1-yl group; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first or twenty-second embodiment.

In a twenty-fourth embodiment of the present disclosure, for the compound of formula (A), or an enantiomer, a diastereomer, and/or a pharmaceutically acceptable salt thereof, B₃ represents a pyrrolidinyl group or a piperazinyl group and the remaining variables are as described in the twenty-third embodiment.

In a twenty-fifth embodiment of the present disclosure, for the compound of formula (A), or an enantiomer, a diastereomer, and/or a pharmaceutically acceptable salt thereof, B₃ represents a piperazinyl group; and the remaining variables are as described in the twenty-third embodiment.

In a twenty-sixth embodiment of the present disclosure, for the compound of formula (A), or an enantiomer, a diastereomer, and/or a pharmaceutically acceptable salt thereof, Ra represents a group selected from the group consisting of:

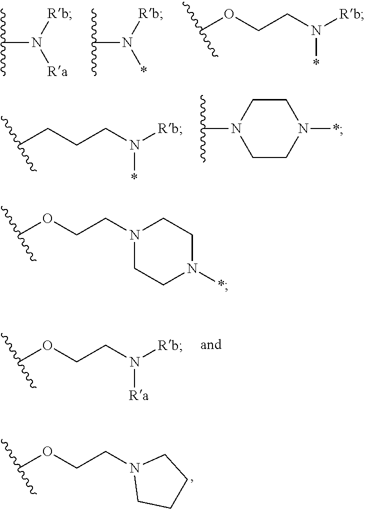

wherein:
-* is a bond to the linker; and
the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first or twenty-second embodiment.

In a twenty-seventh embodiment of the present disclosure, for the compound of formula (A), or an enantiomer, a diastereomer, and/or a pharmaceutically acceptable salt thereof, R₈ represents a group selected from:

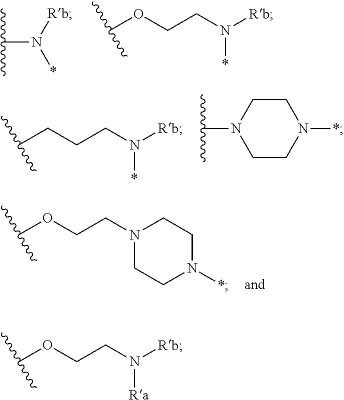

wherein:
-* is a bond to the linker; and
the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first or twenty-second embodiment.

In a twenty-eighth embodiment of the present disclosure, for the compound of formula (A), or an enantiomer, a diastereomer, and/or a pharmaceutically acceptable salt thereof:
for formula (IB) or (IC-1), R₃ represents a group selected from the group consisting of: hydrogen; a linear or branched C₁-C₆alkyl; —X₁—NRₐR_{b}; —X₁—N₃ and
   —X₁-≡CH,
for formula (IIB) or (IIC-1), Z₁ represents a bond, and R₃ represents hydrogen,
for formula (IC-1), G is selected from the group consisting of —C(O)OH and —C(O)N(CH₃)₂;
for formula (IIC-1), G is selected from the group consisting of —C(O)NHS(O)₂H, —C(O)NH₂, —C(O)NHCH₃, —C(O)NHC(CH₃)₂, —C(O)N(CH₃)₂, —C(O)OH, and —CH₂OH;
R₆ represents a linear or branched —C₁-C₆alkylene-Rₐ group, —X₂—O—R₇ or a heteroarylene-R₇ group optionally substituted by a linear or branched C₁-C₆alkyl group,
R₇ represents a group selected from the group consisting of:

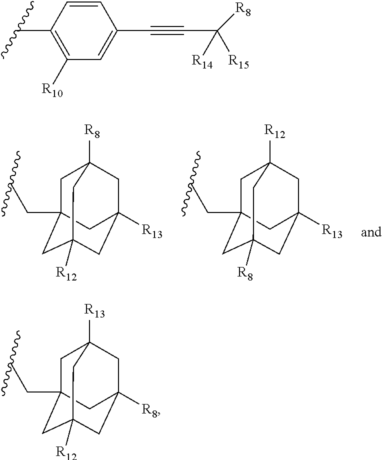

R₈ represents a group selected from the group consisting of: —NR′ₐR′_{b}; and —O—X′₂—NR′ₐR′_{b},
R₁₀ represents fluorine,
R₁₂ and R₁₃, independently of one another, represent a hydrogen atom or a methyl group,
R₁₄ and R₁₅, independently of one another, represent a hydrogen or a methyl group,
X₁ and X₂ independently of one another, represent a linear or branched C₁-C₆alkylene group optionally substituted by one or two groups selected from the group consisting of trifluoromethyl, hydroxyl, a halogen, and C₁-C₆alkoxy,
X′₂ represents a linear or branched C₁-C₆alkylene,
R′ₐ and R′_{b} independently of one another, represent a group selected from the group consisting of: hydrogen; a linear or branched C₁-C₆alkyl optionally substituted by one or two hydroxyl or C₁-C₆alkoxy groups; and a C₁-C₆alkylene-NR′_{d}R′ₑ; or R′ₐ and R′_{b} form with the nitrogen atom carrying them a cycle B₃,
R′_{d}, R′ₑ independently of one another, represents a hydrogen or a linear or branched C₁-C₆alkyl group,
B₃ represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen and nitrogen, (iii) be substituted by one or two groups selected from the group consisting of: fluorine, bromine, chlorine, linear or a branched C₁-C₆alkyl, hydroxyl, and oxo; and
the remaining variables are as described in the nineteenth embodiment.

In a twenty-ninth embodiment of the present disclosure, for the compound of formula (IB), (IC), (IIB) or (IIC), or an enantiomer, a diastereomer, and/or a pharmaceutically acceptable salt thereof, the variables are defined as:
for formula (IB) or (IC), R₃ represents a group selected from: hydrogen; linear or branched C₁-C₆alkyl; —X₁—N₃ and —X₁-≡CH,
for formula (IIB) or (IIC), Z₁ represents a bond, and R₃ represents hydrogen,
R₆ represents a linear or branched —C₁-C₆alkylene-Rₐ group, —X₂—O—R₇ or a heteroarylene-R₇ group optionally substituted by a linear or branched C₁-C₆alkyl group,
R₇ represents a group selected from:

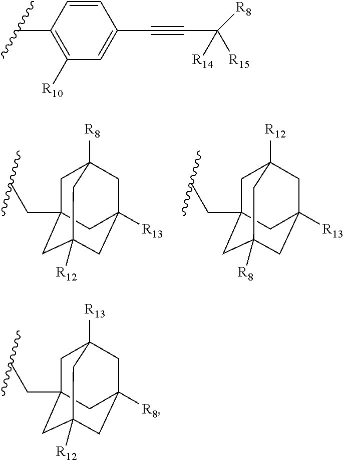

R₈ represents a group selected from: —NR′ₐR′_{b}; and —O—X′₂—NR′ₐR′_{b},
R₁₀ represents fluorine,
R₁₂ and R₁₃, independently of one another, represent a hydrogen atom or a methyl group,
R₁₄ and R₁₅, independently of one another, represent a hydrogen or a methyl group,
X₁ and X₂ independently of one another, represent a linear or branched C₁-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; linear or branched C₁-C₆alkyl optionally substituted by one or two hydroxyl or C₁-C₆alkoxy groups; C₁-C₆alkylene-NR′_{d}R′e;
   or R′ₐ and R′_{b} form with the nitrogen atom carrying them a cycle B₃,
R′_{d}, R′ₑ independently of one another, represents a hydrogen or a linear or branched C₁-C₆alkyl group,
   B₃ represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen and nitrogen, (iii) be substituted by one or two groups selected from: fluorine, bromine, chlorine, linear or branched C₁-C₆alkyl, hydroxyl, and oxo; and
the remaining variables are as described in the twentieth embodiment.

In a thirtieth embodiment of the present disclosure, for the compound of formula (A), or an enantiomer, a diastereomer, and/or a pharmaceutically acceptable salt thereof, B₃ represents pyrrolidinyl group or a piperazinyl group and the remaining variables are as described in the twenty-eighth or twenty-ninth embodiment

In a thirty-first embodiment of the present disclosure, for the compound of formula (A), (IB), (IC), (IIB), or (IIC), or an enantiomer, a diastereomer, and/or a pharmaceutically acceptable salt thereof, B₃ represents a piperazinyl group; and the remaining variables are as described in the twenty-eighth or twenty-ninth embodiment.

In a thirty-second embodiment of the present disclosure, for the compound of formula (A), or an enantiomer, a diastereomer, and/or a pharmaceutically acceptable salt thereof, Ra represents a group selected from the group consisting of:

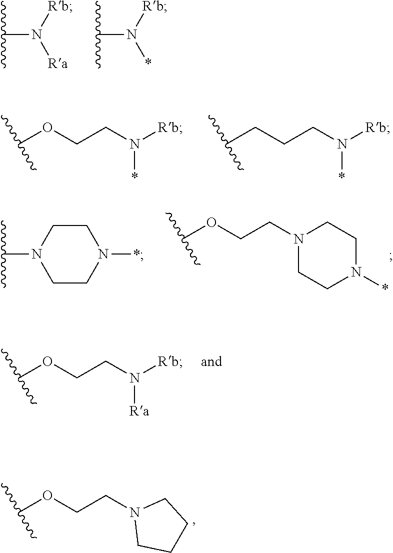

wherein:
-* is a bond to the linker; and
   the remaining variables are as described in the twenty-eighth or twenty-ninth embodiment.

In a thirty-third embodiment of the present disclosure, for the compound of formula (IB), (IC), (IIB), or (IIC), or an enantiomer, a diastereomer, and/or a pharmaceutically acceptable salt thereof, R₈ represents a group selected from:

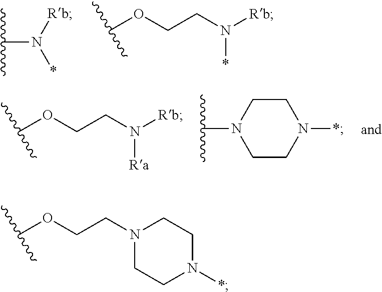

wherein:
-* is a bond to the linker; and
the remaining variables are as described in the twenty-eighth or twenty-ninth embodiment.

In a thirty-fourth embodiment of the present disclosure, for the compound of formula (A), or an enantiomer, a diastereomer, and/or a pharmaceutically acceptable salt thereof, D represents any one of the following attached to L:

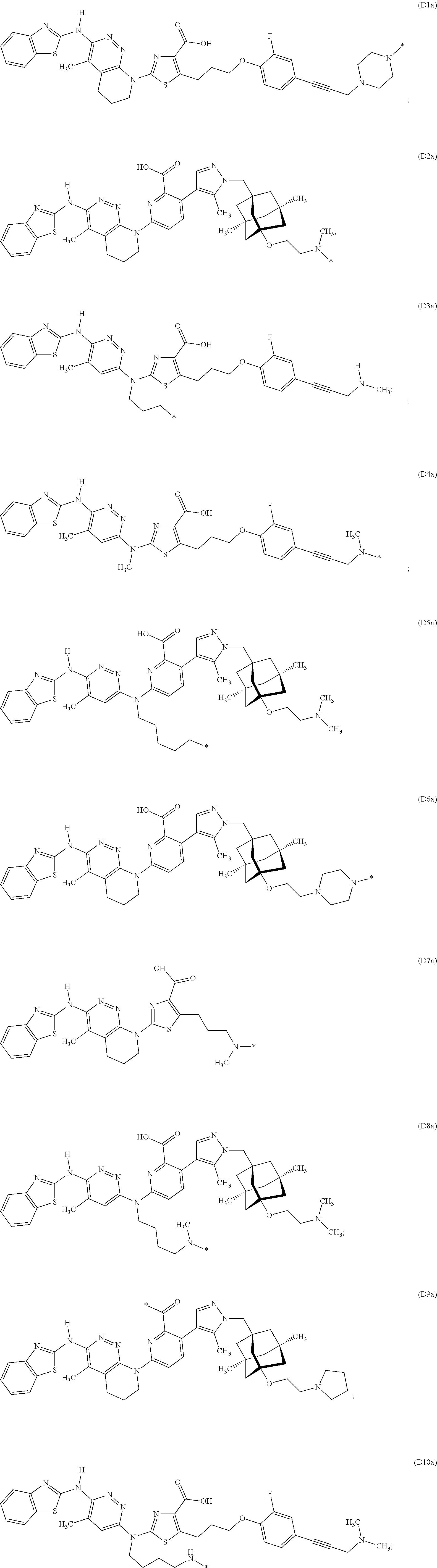

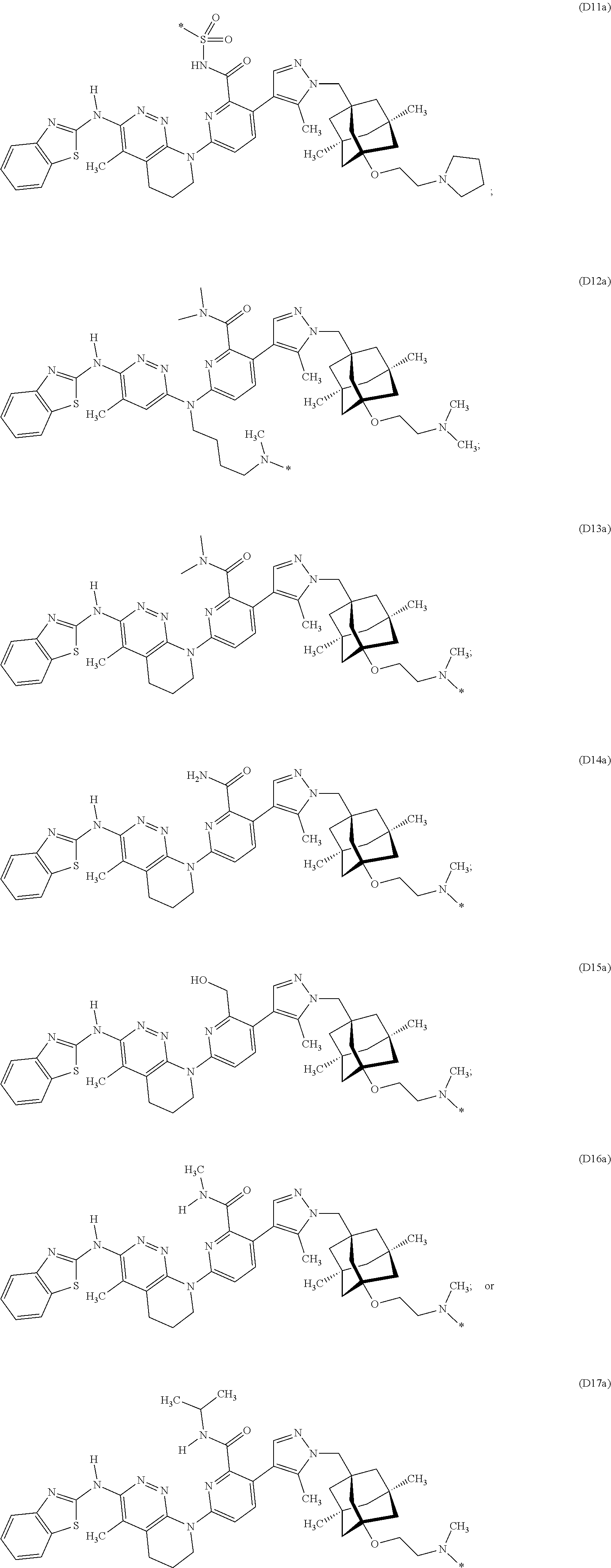

or an enantiomer, a diastereoisomer, and/or a pharmaceutically acceptable salt of any one of the foregoing, wherein:
-* is a bond to the linker;
   the remaining variables are as described in the first, second, third, fourth, fifth, sixth, seventh or eighth embodiment.

In a thirty-fifth embodiment of the present disclosure, for the compound of formula (A), or an enantiomer, a diastereomer, and/or a pharmaceutically acceptable salt thereof, D-L in Formula (A) comprises a formula selected from:

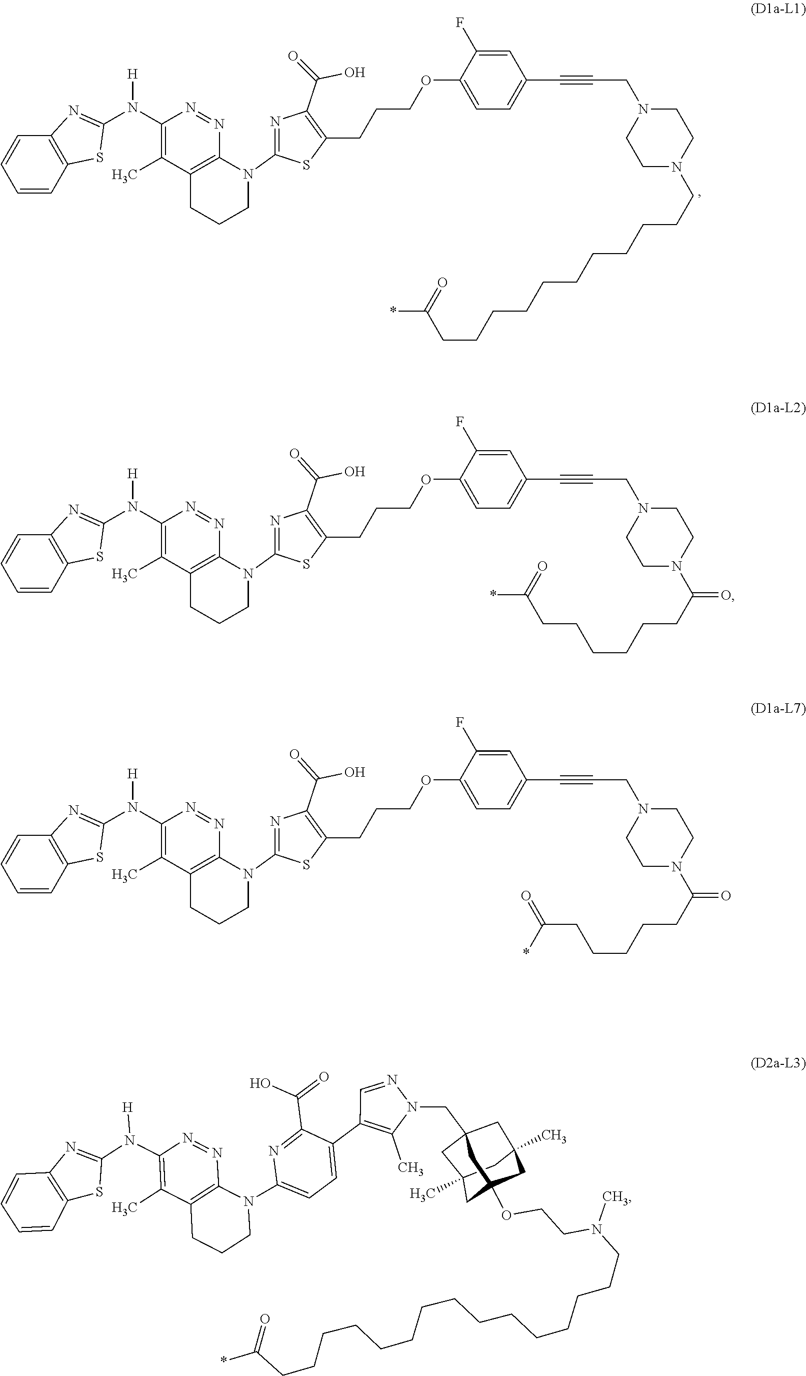

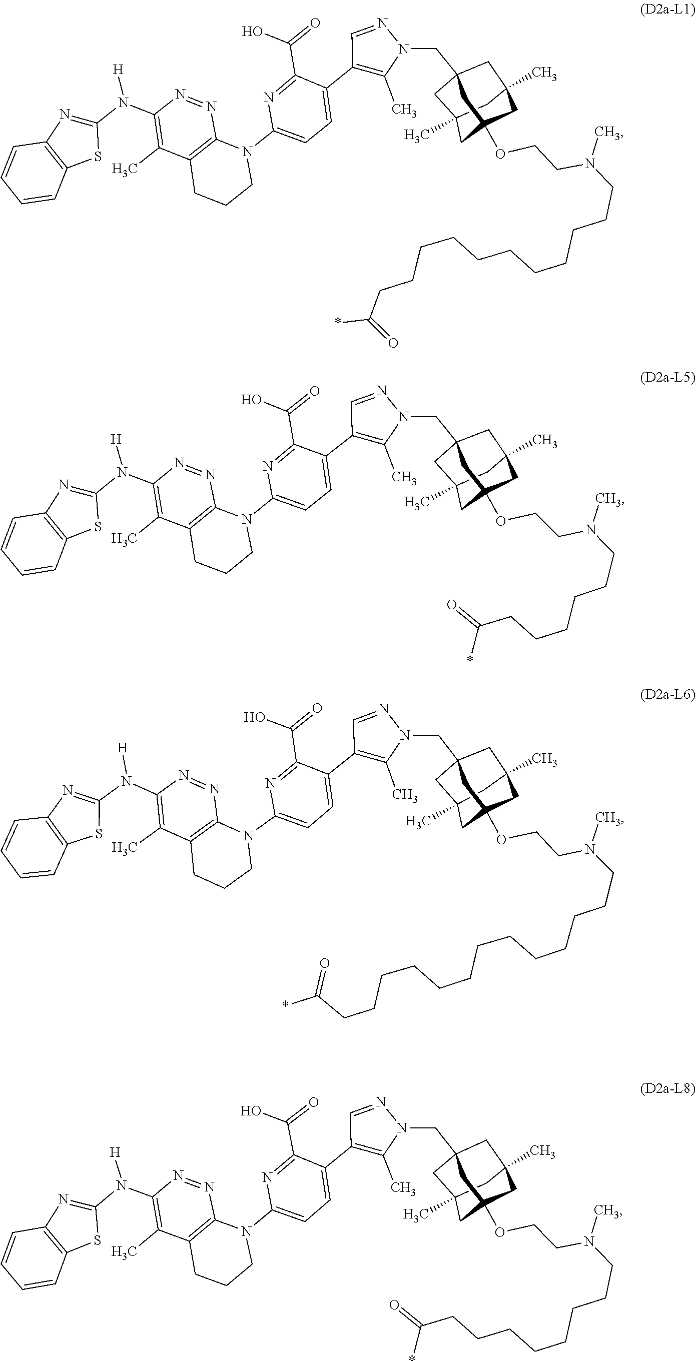

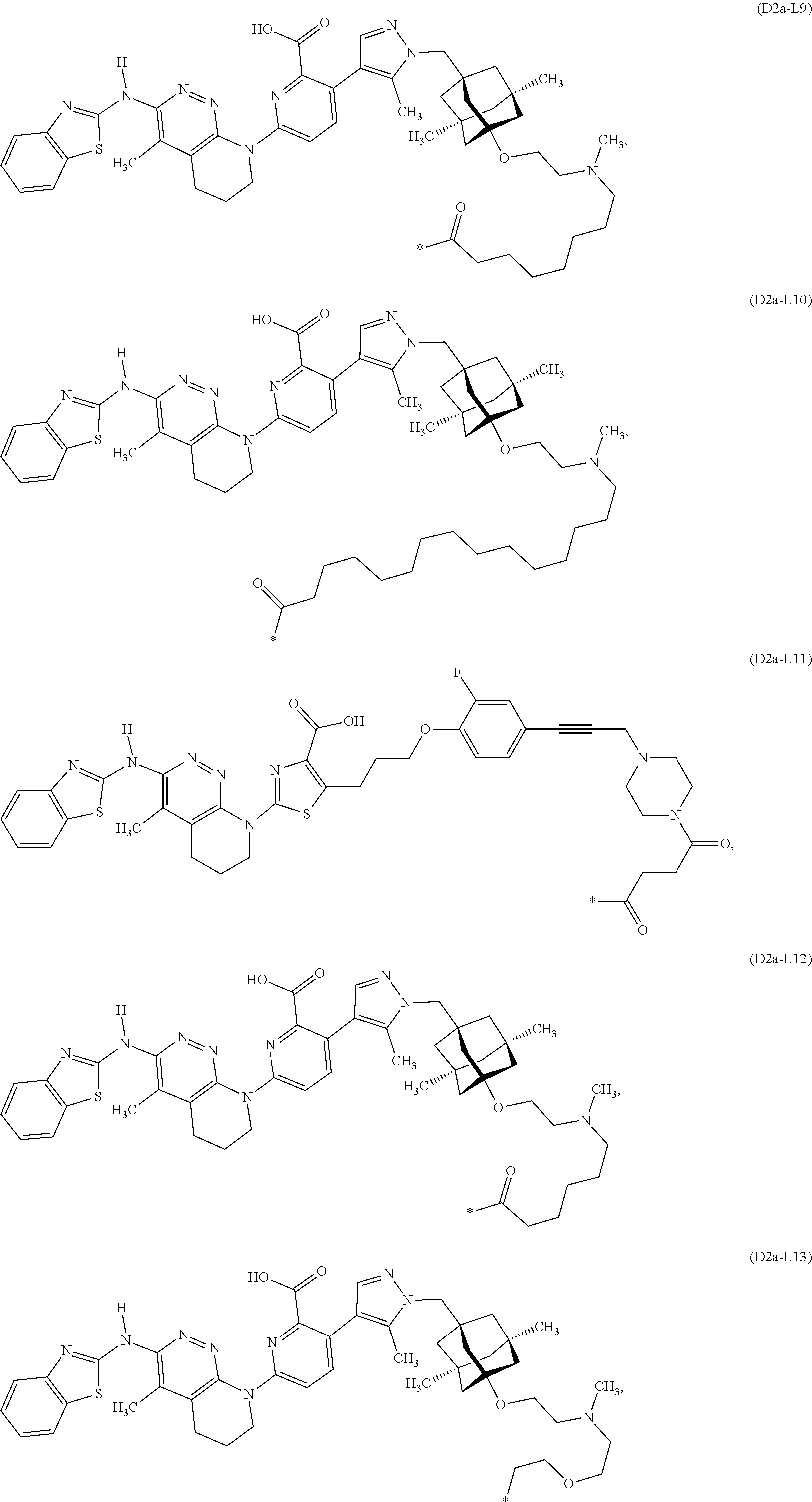

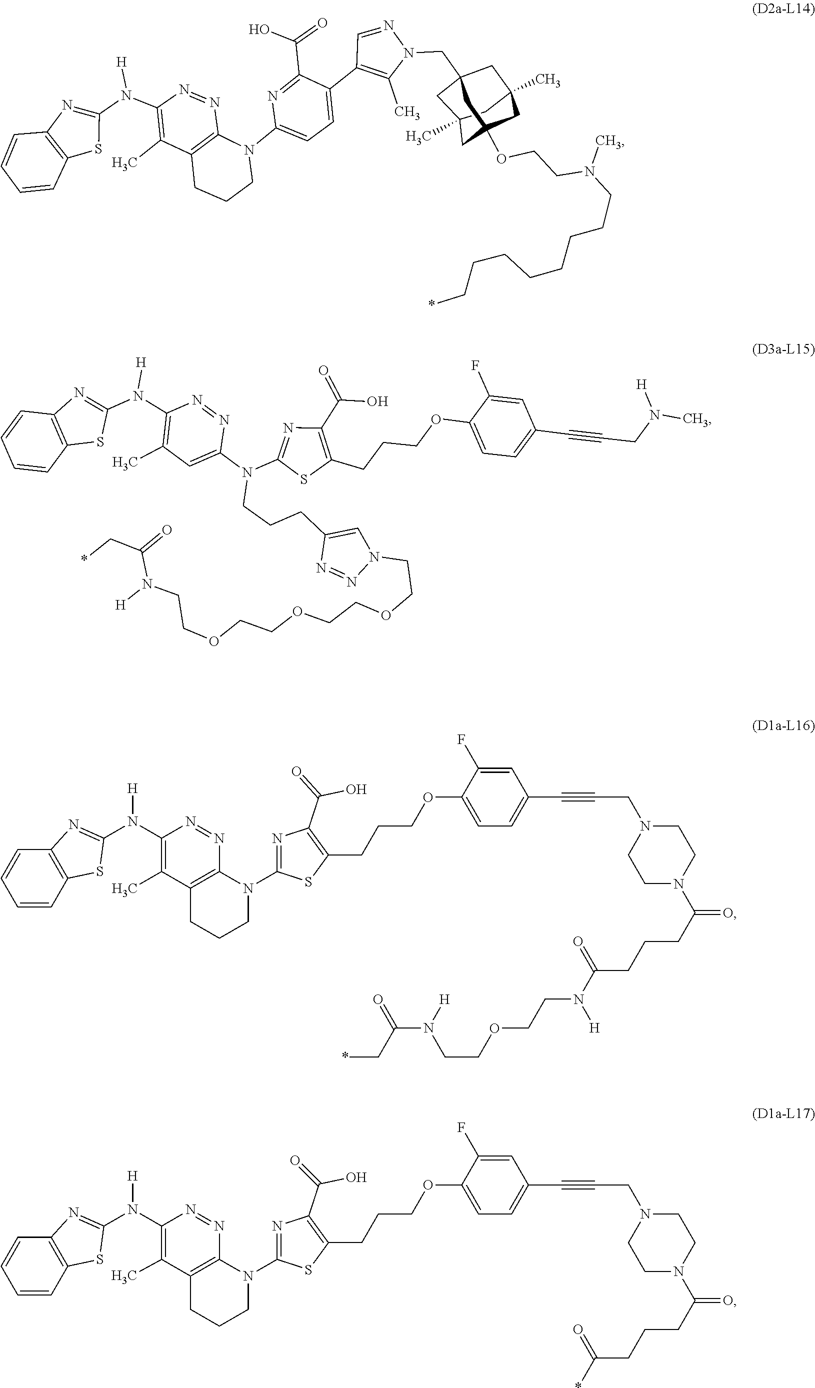

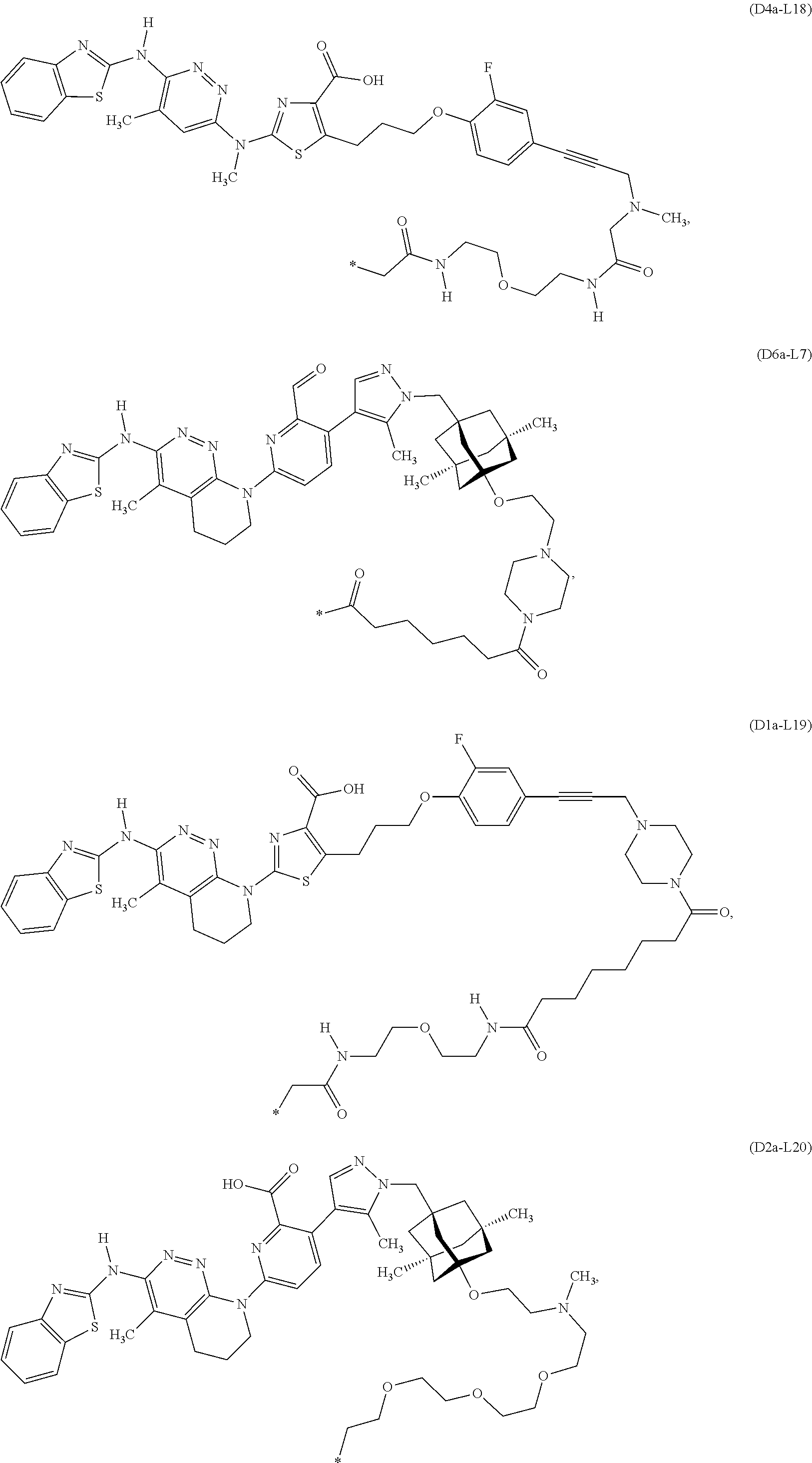

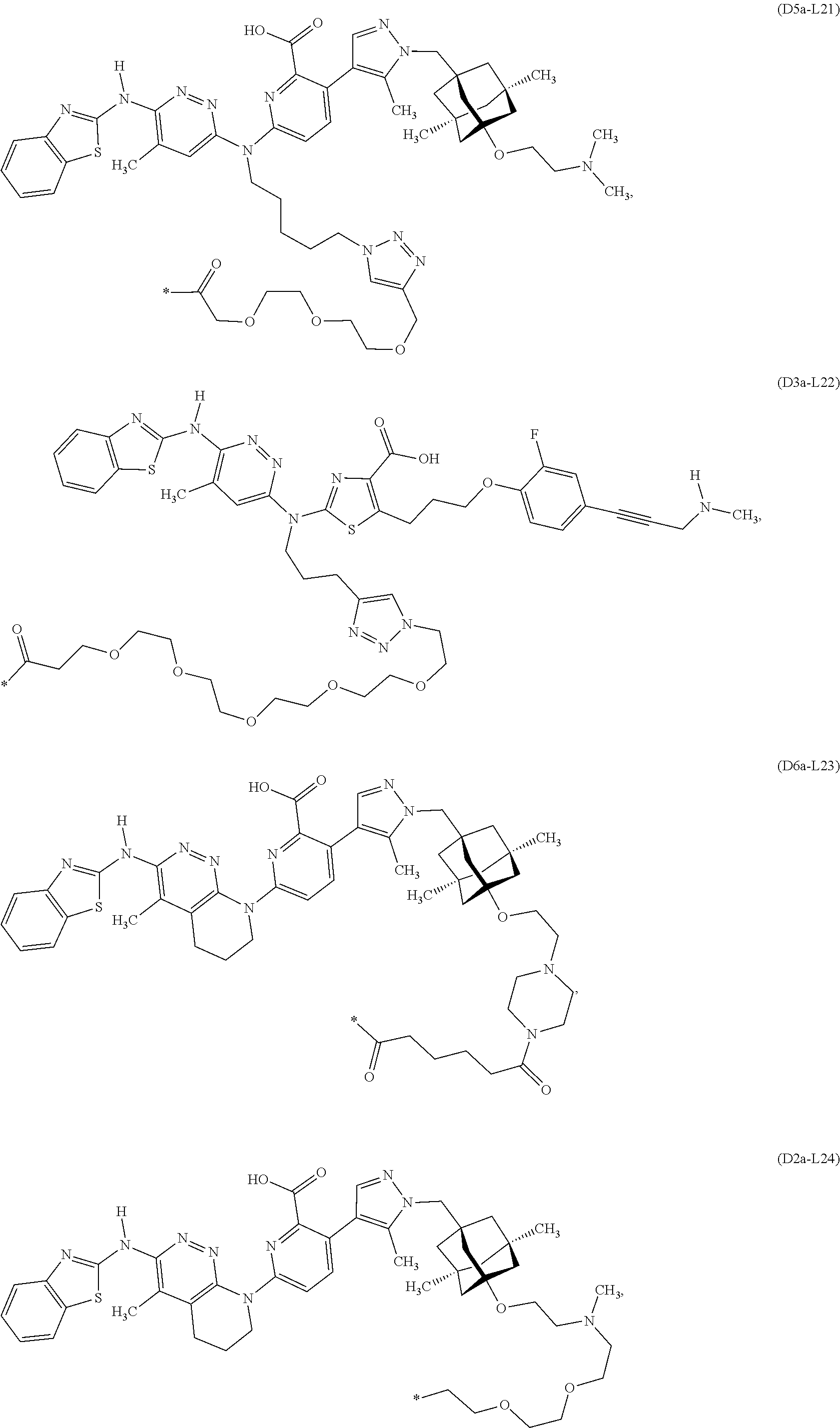

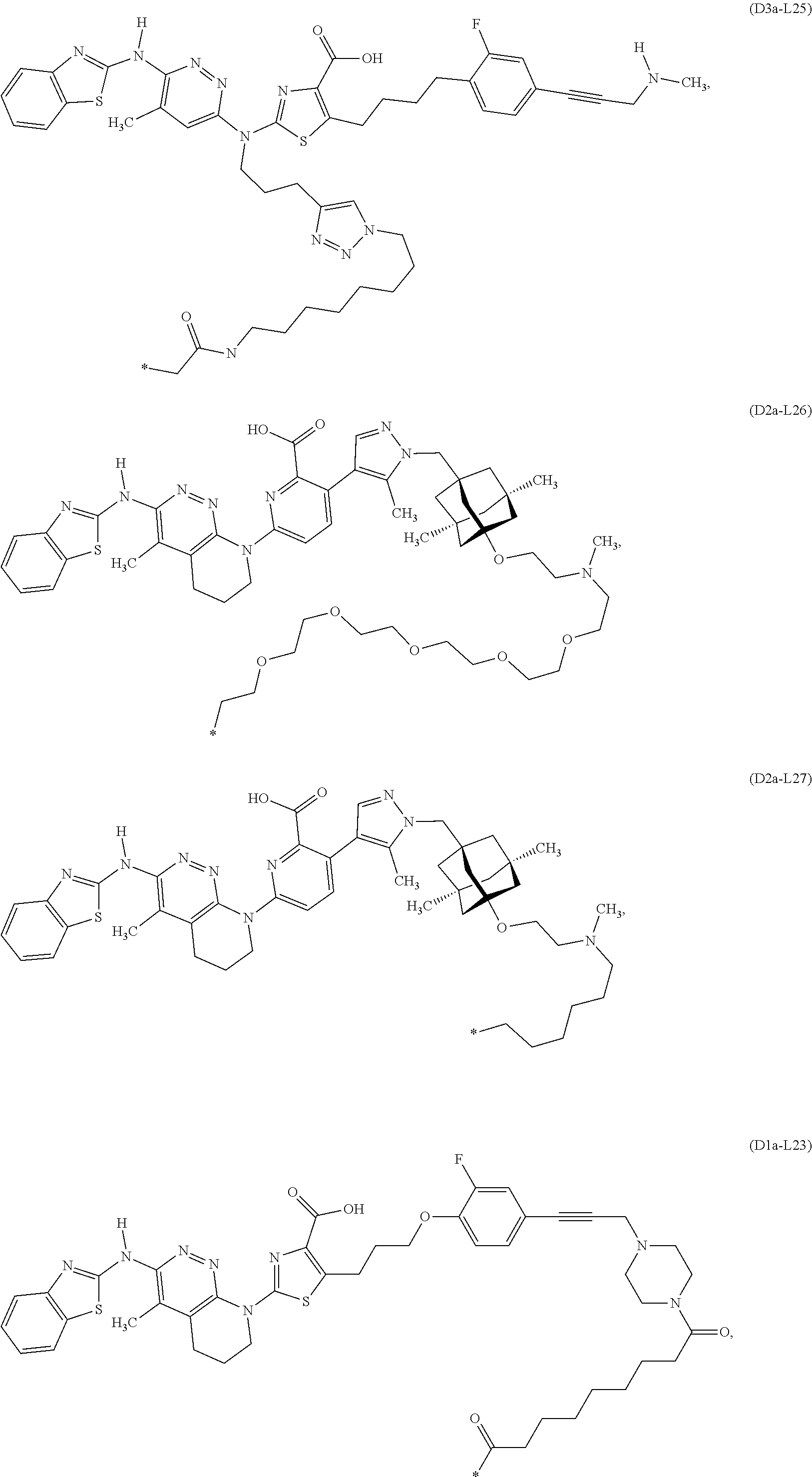

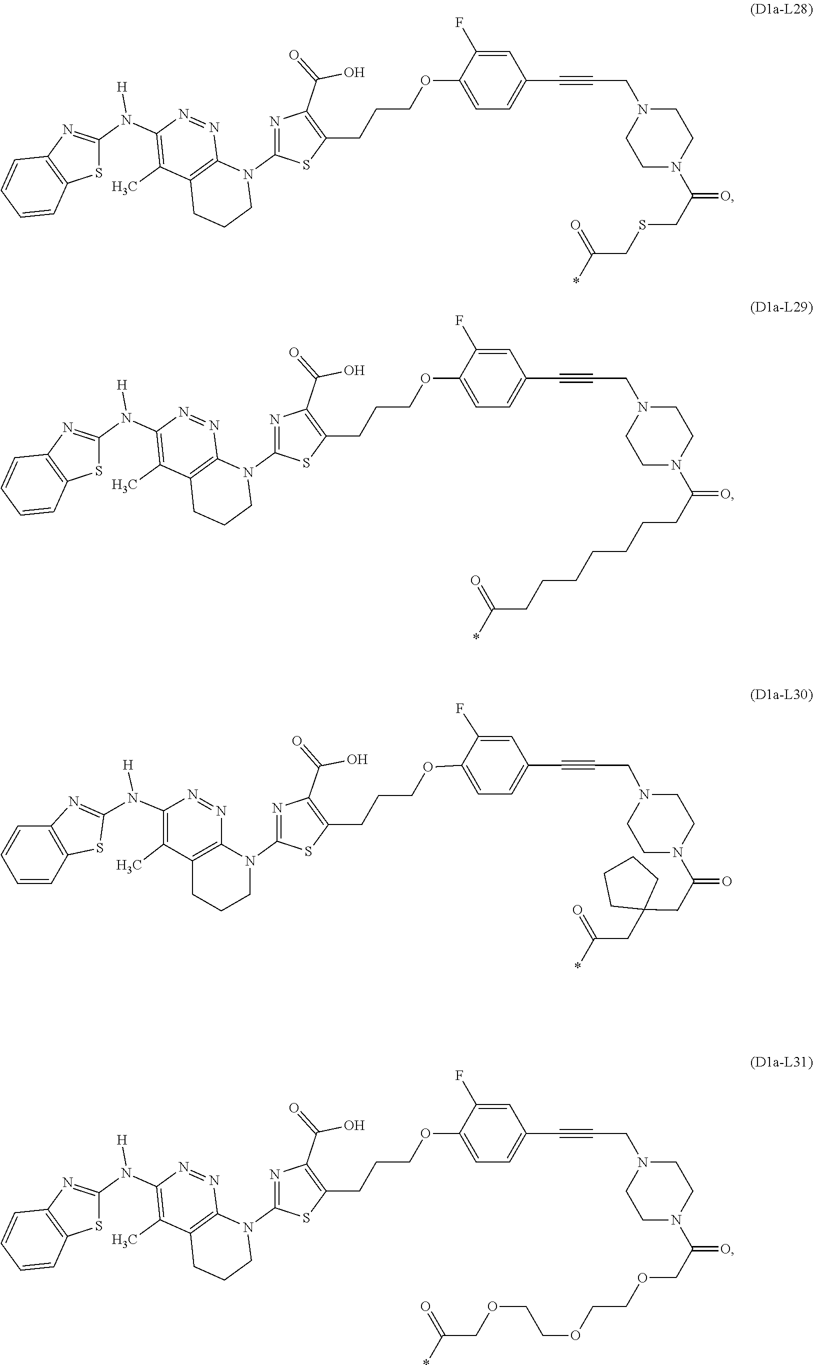 [SMILES: *C(=O)CSCC(=O)N1CCN(CC#Cc2ccc(OCCCc3sc(N4CCCc5c4nnc(Nc4nc6ccccc6s4)c5C)nc3C(=O)O)c(F)c2)CC1 ; *C(=O)CCCCCCCC(=O)N1CCN(CC#Cc2ccc(OCCCc3sc(N4CCCc5c4nnc(Nc4nc6ccccc6s4)c5C)nc3C(=O)O)c(F)c2)CC1 ; *C(=O)CC1(CC(=O)N2CCN(CC#Cc3ccc(OCCCc4sc(N5CCCc6c5nnc(Nc5nc7ccccc7s5)c6C)nc4C(=O)O)c(F)c3)CC2)CCCC1 ; *C(=O)COCCOCCOCC(=O)N1CCN(CC#Cc2ccc(OCCCc3sc(N4CCCc5c4nnc(Nc4nc6ccccc6s4)c5C)nc3C(=O)O)c(F)c2)CC1]

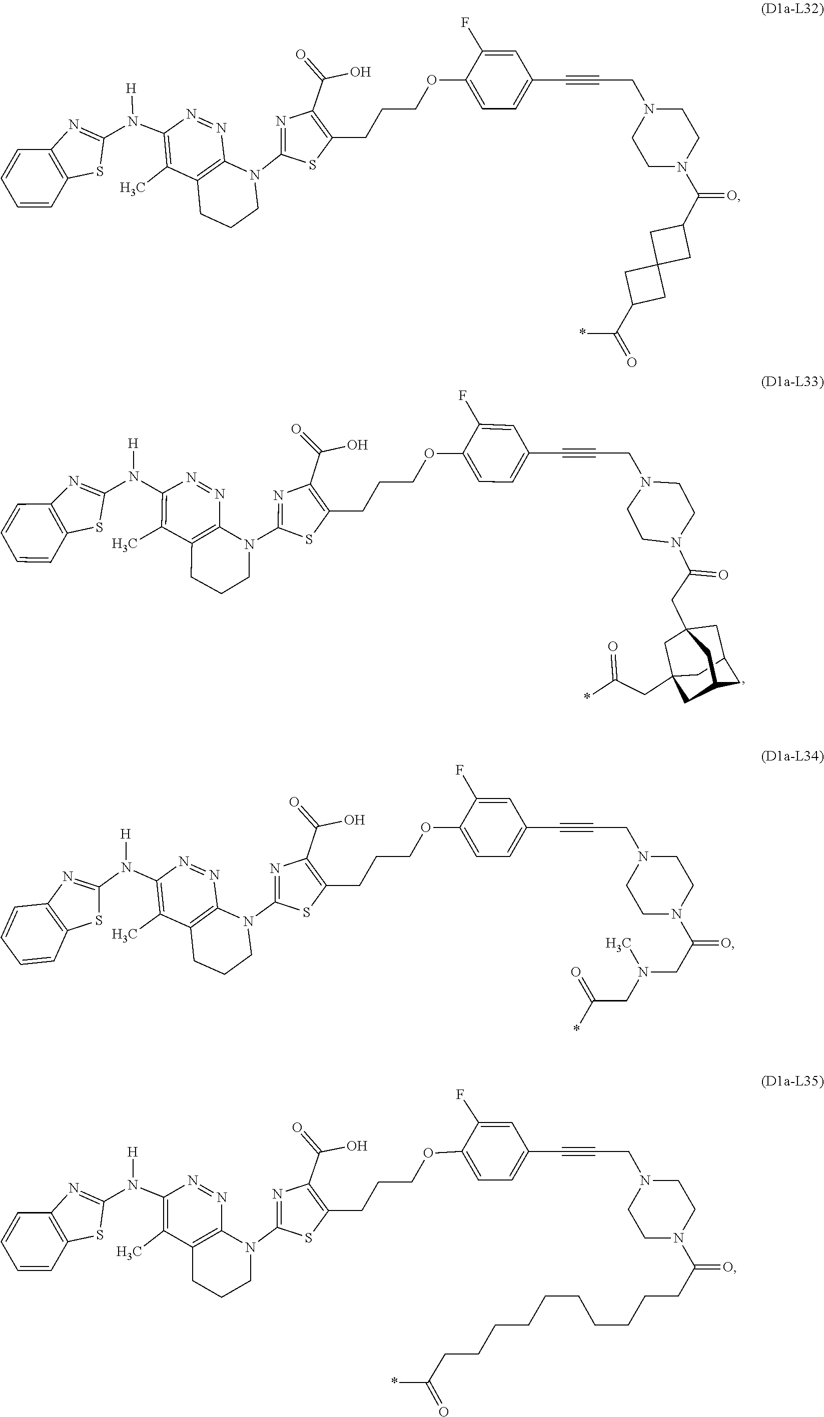

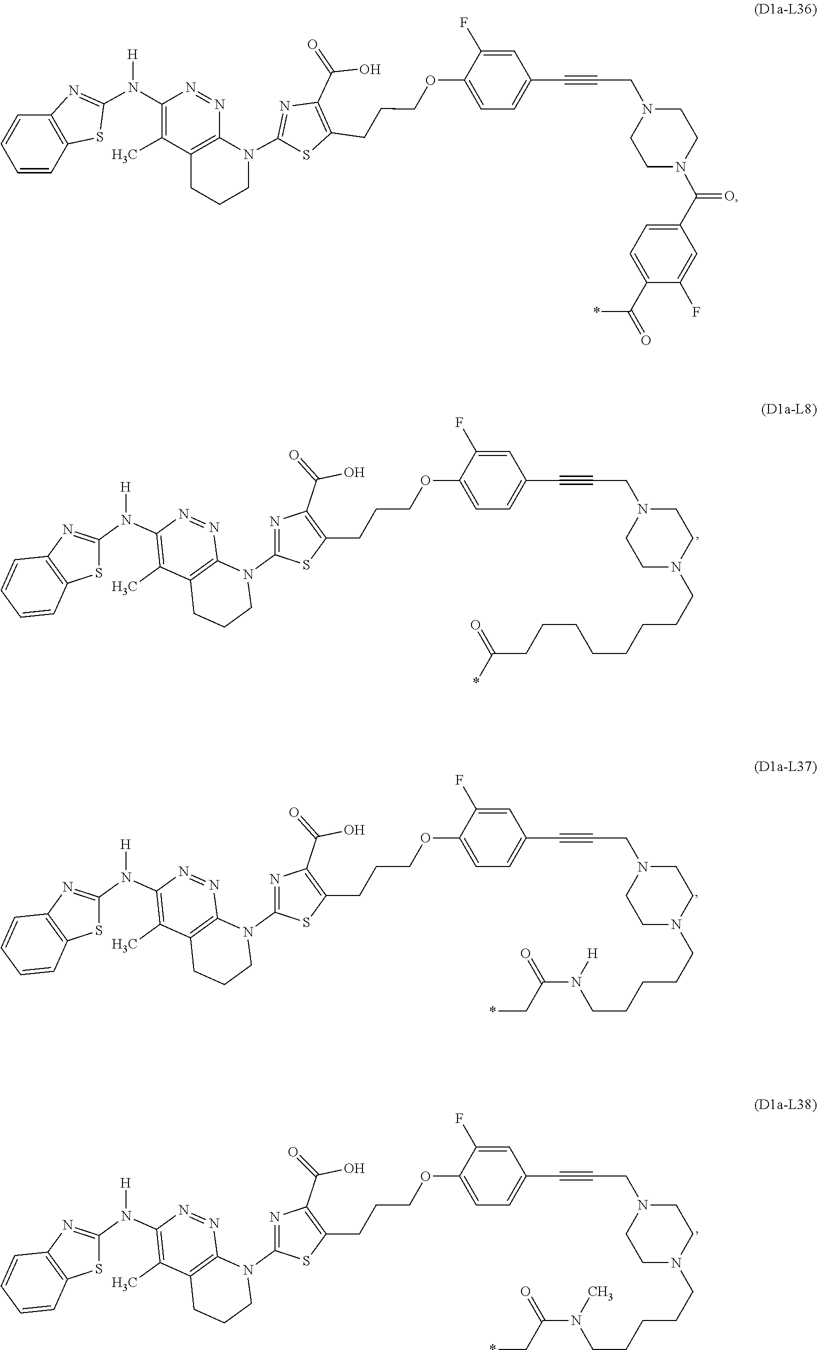 [SMILES: *C(=O)c1ccc(C(=O)N2CCN(CC#Cc3ccc(OCCCc4sc(N5CCCc6c5nnc(Nc5nc7ccccc7s5)c6C)nc4C(=O)O)c(F)c3)CC2)cc1F ; *C(=O)CCCCCCCCN1CCN(CC#Cc2ccc(OCCCc3sc(N4CCCc5c4nnc(Nc4nc6ccccc6s4)c5C)nc3C(=O)O)c(F)c2)CC1 ; *CC(=O)NCCCCCN1CCN(CC#Cc2ccc(OCCCc3sc(N4CCCc5c4nnc(Nc4nc6ccccc6s4)c5C)nc3C(=O)O)c(F)c2)CC1 ; *CC(=O)N(C)CCCCCN1CCN(CC#Cc2ccc(OCCCc3sc(N4CCCc5c4nnc(Nc4nc6ccccc6s4)c5C)nc3C(=O)O)c(F)c2)CC1]

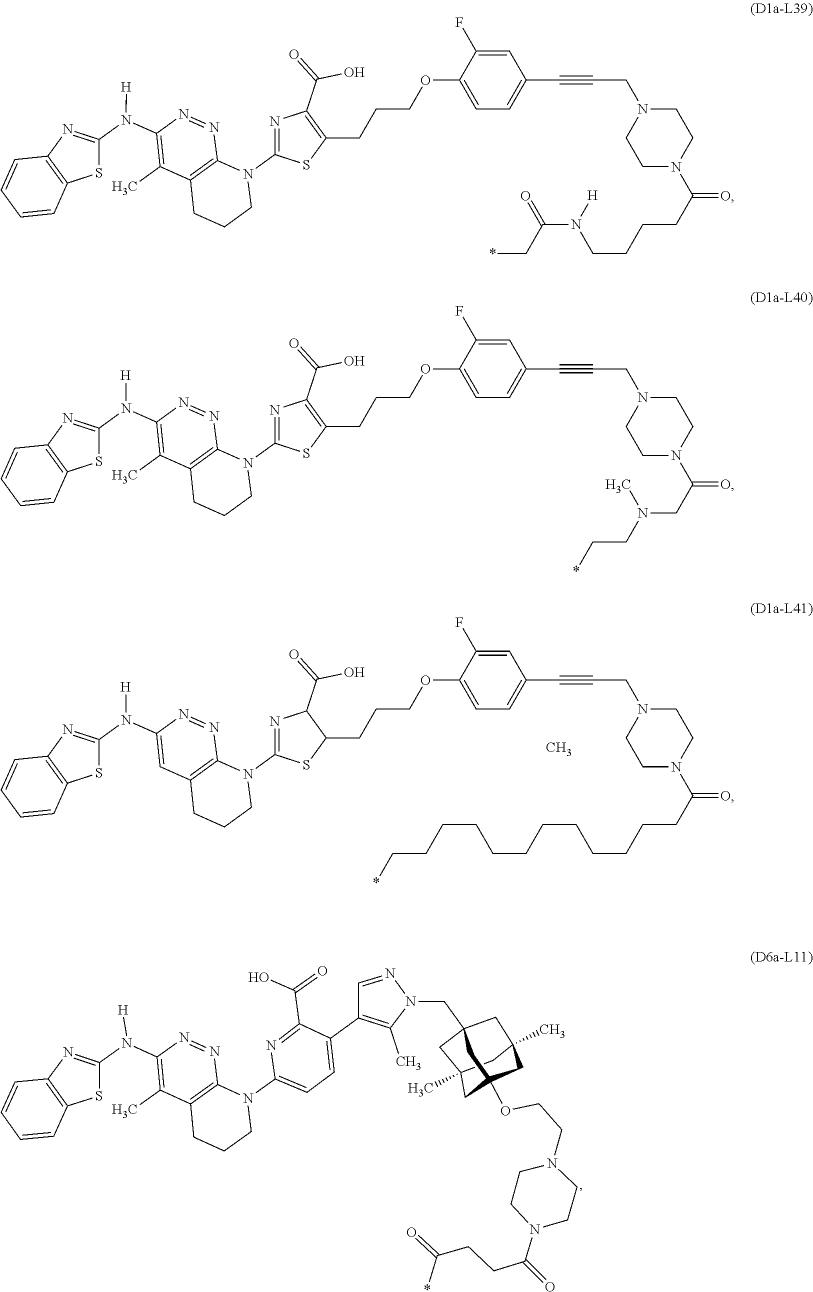

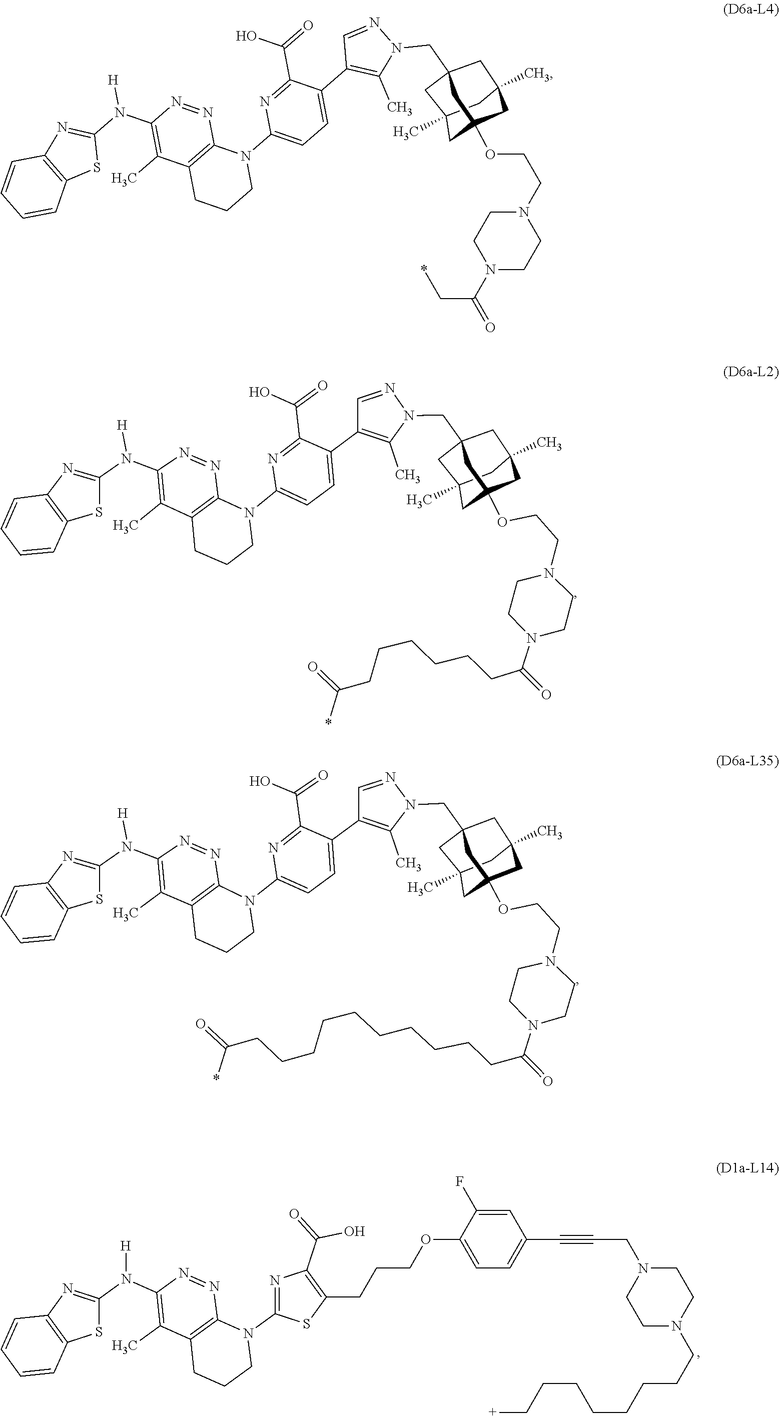

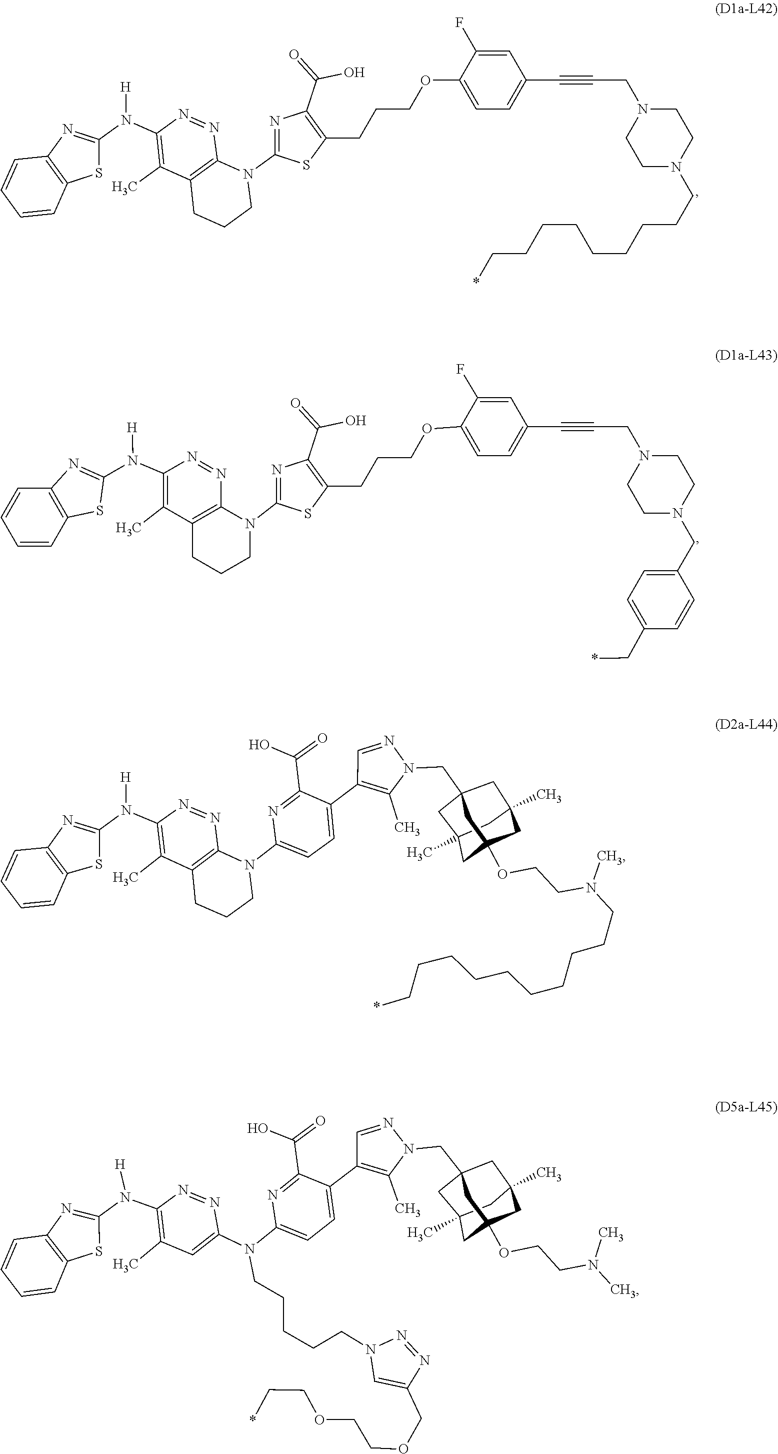

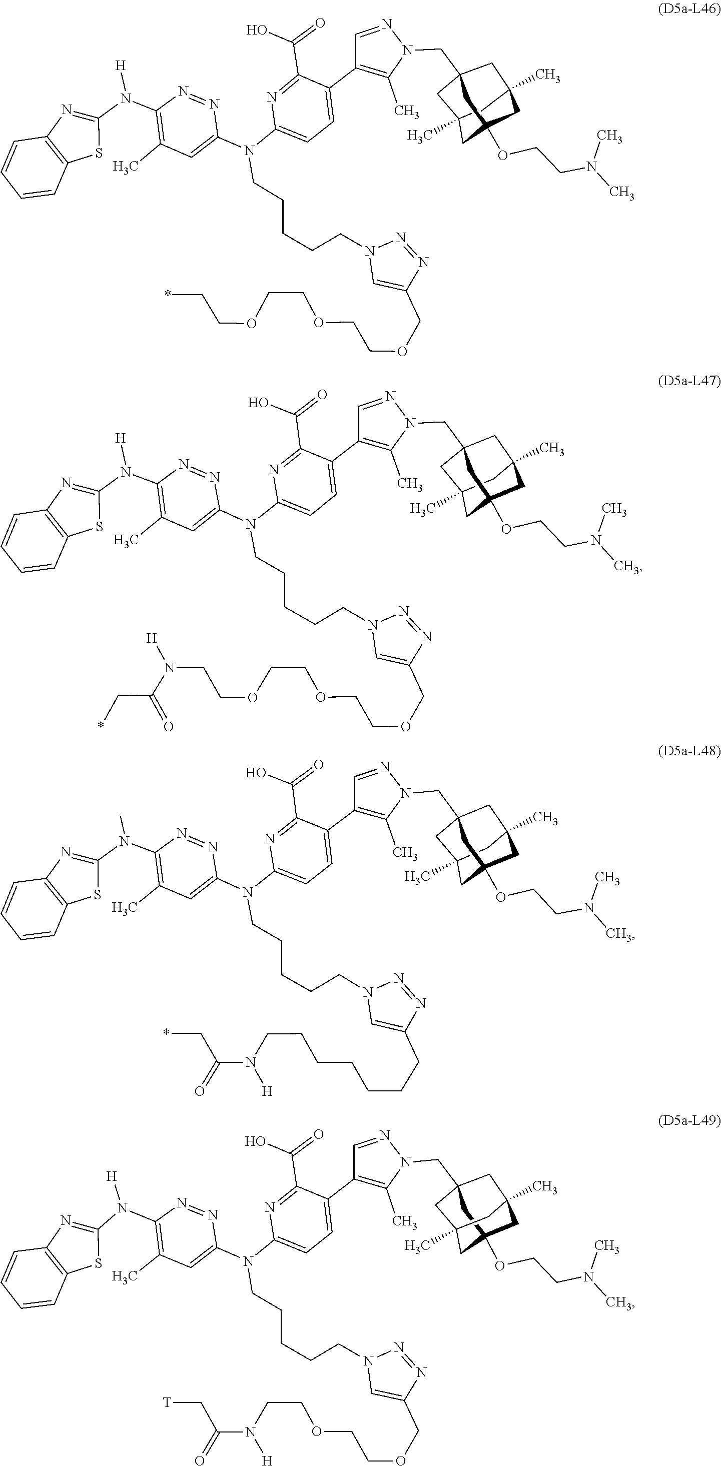

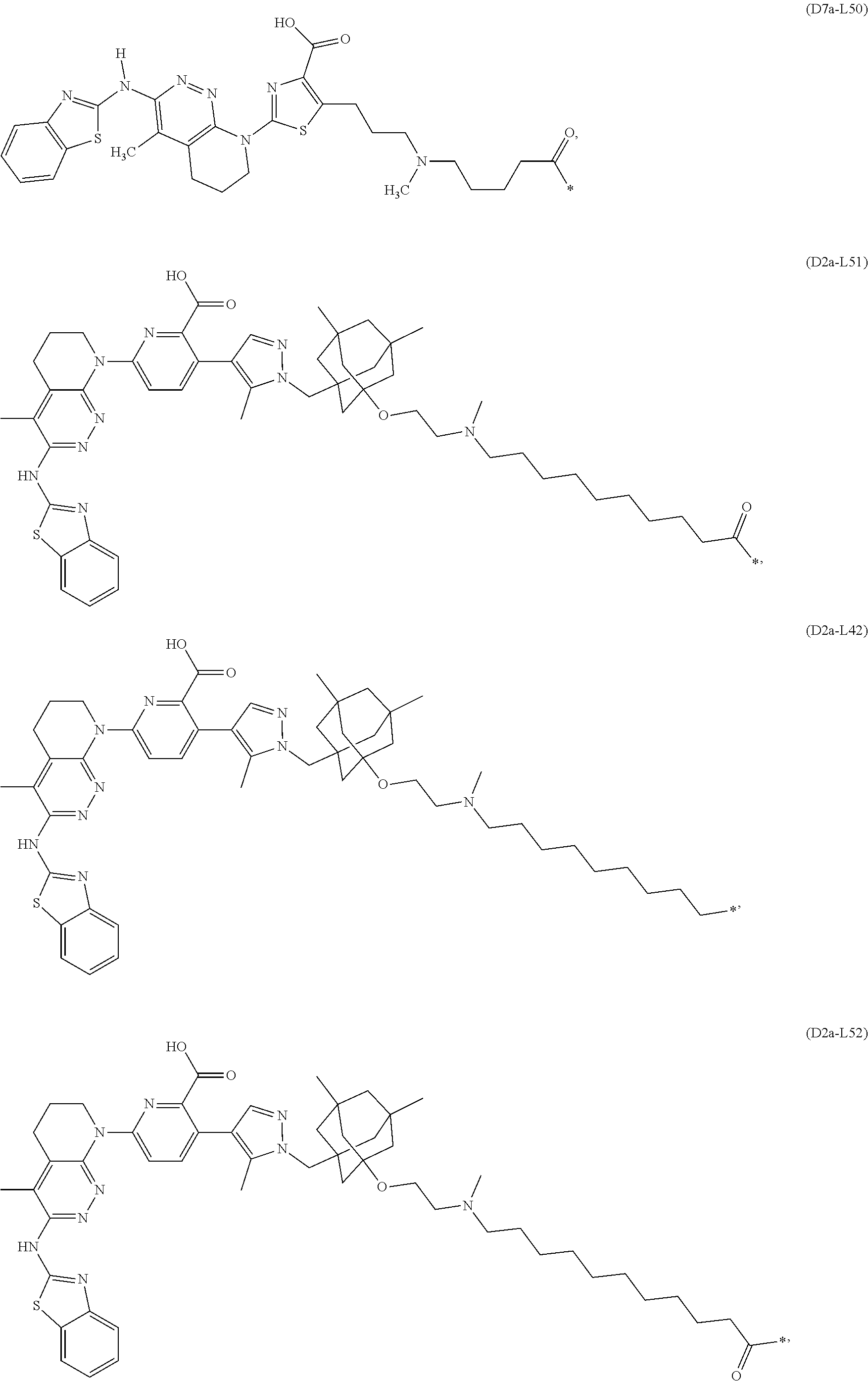 [SMILES: *C(=O)CCCCN(C)CCCc1sc(N2CCCc3c2nnc(Nc2nc4ccccc4s2)c3C)nc1C(=O)O ; *C(=O)CCCCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2 ; *CCCCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2 ; *C(=O)CCCCCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2]

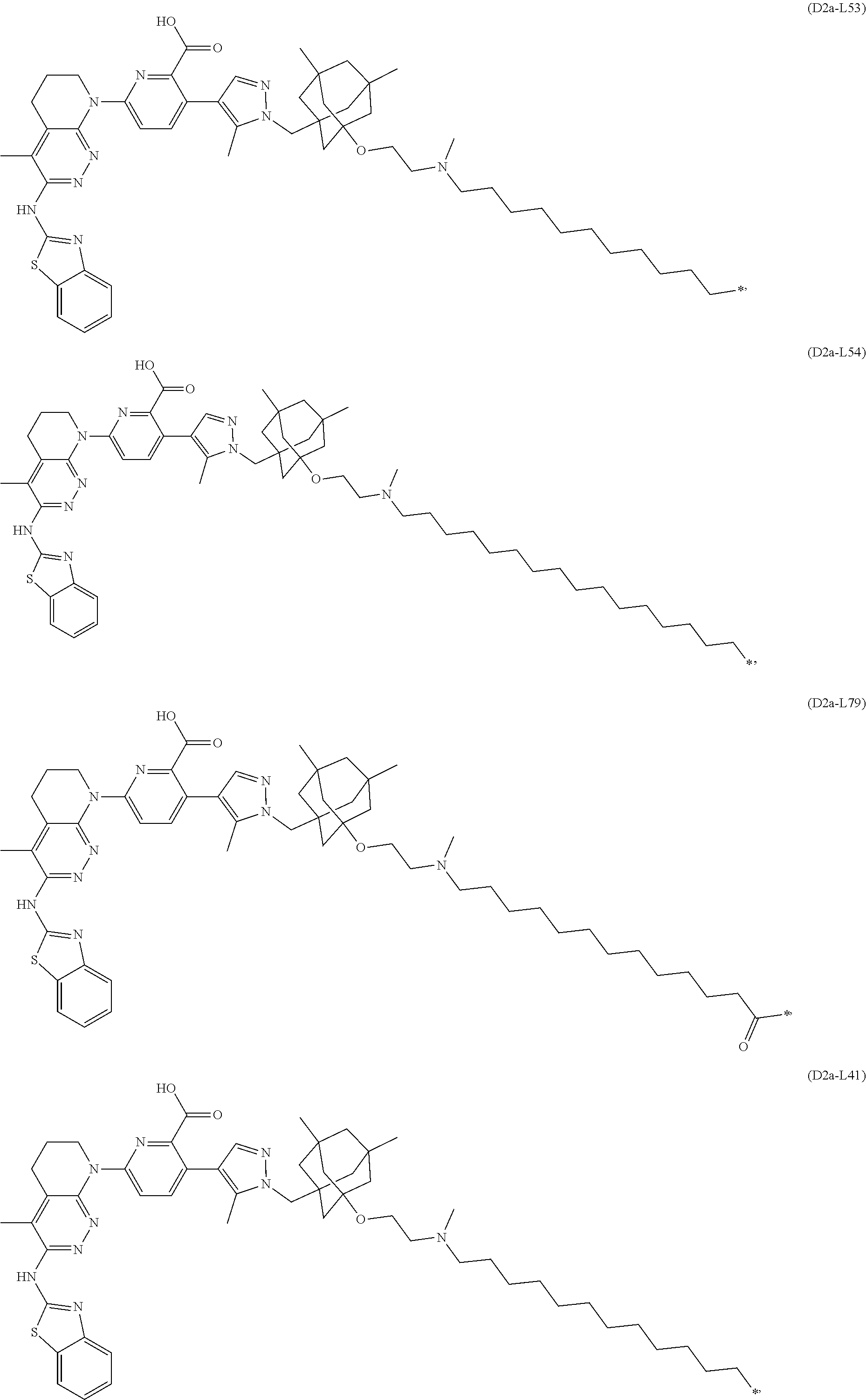

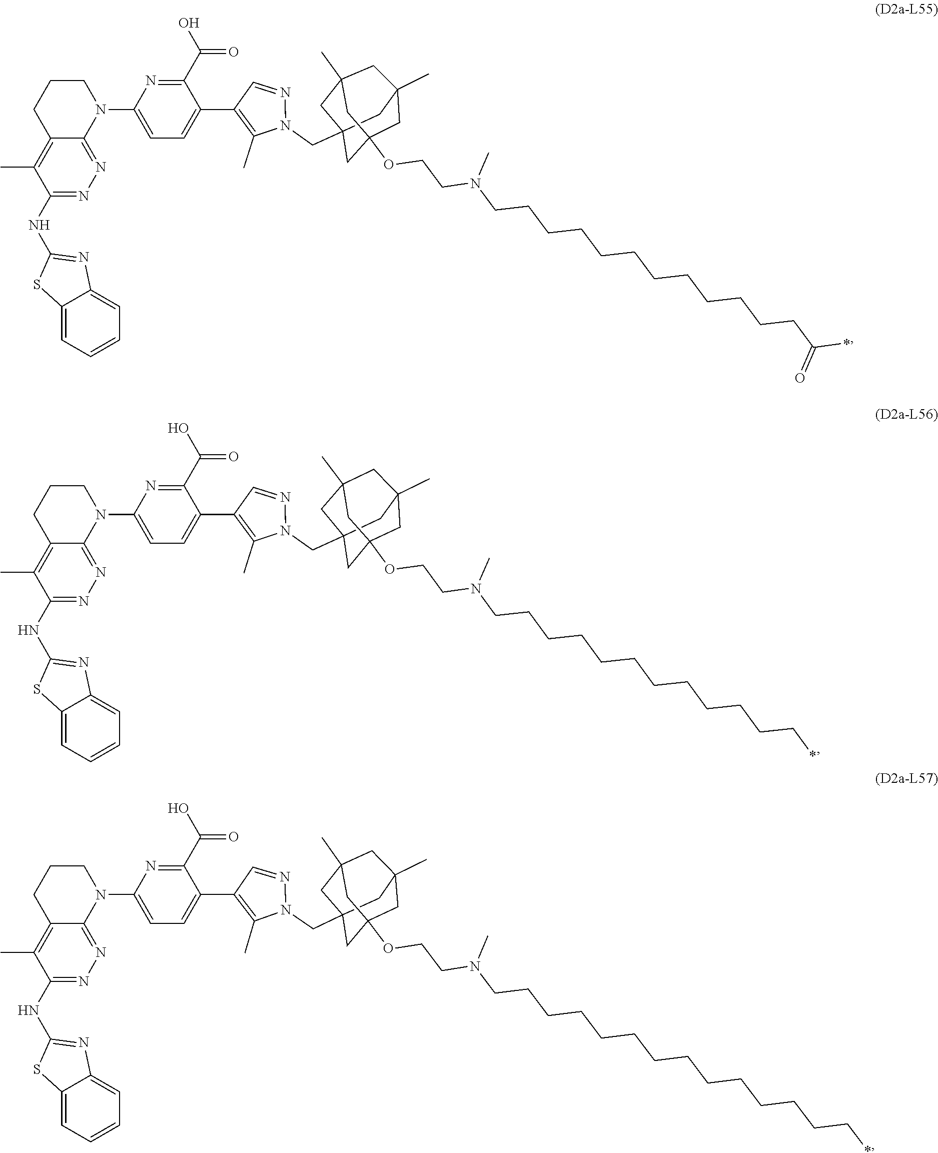

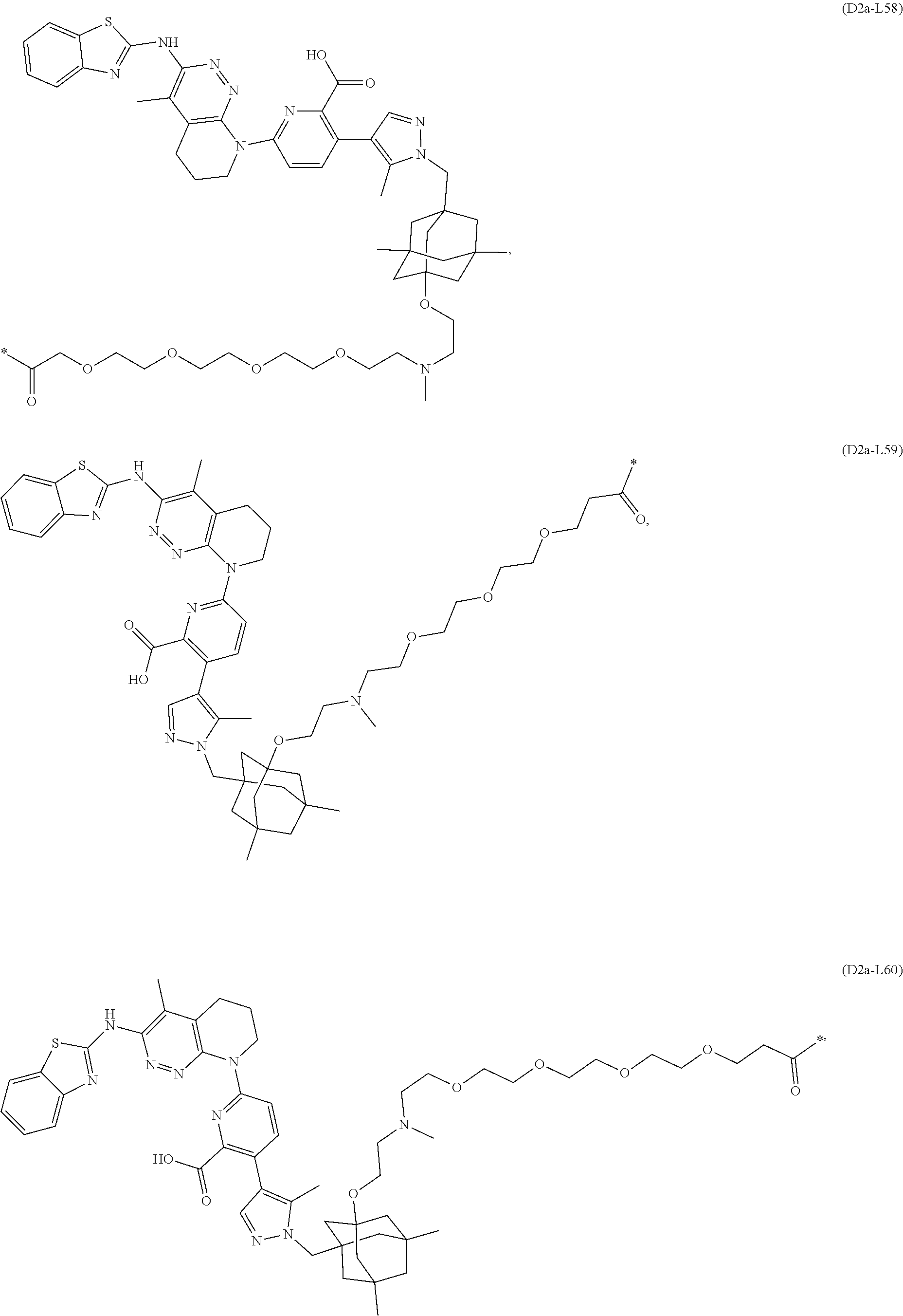

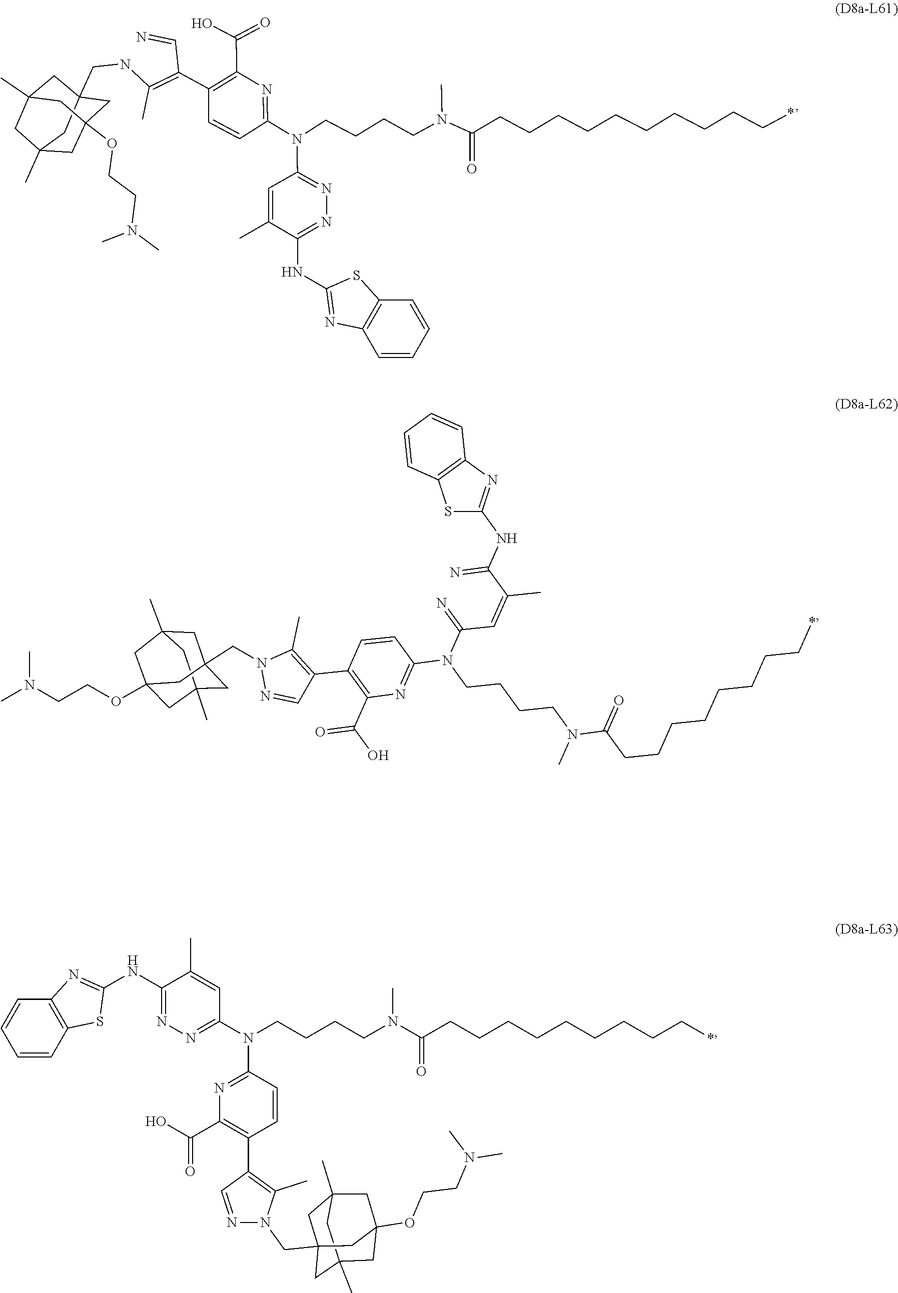

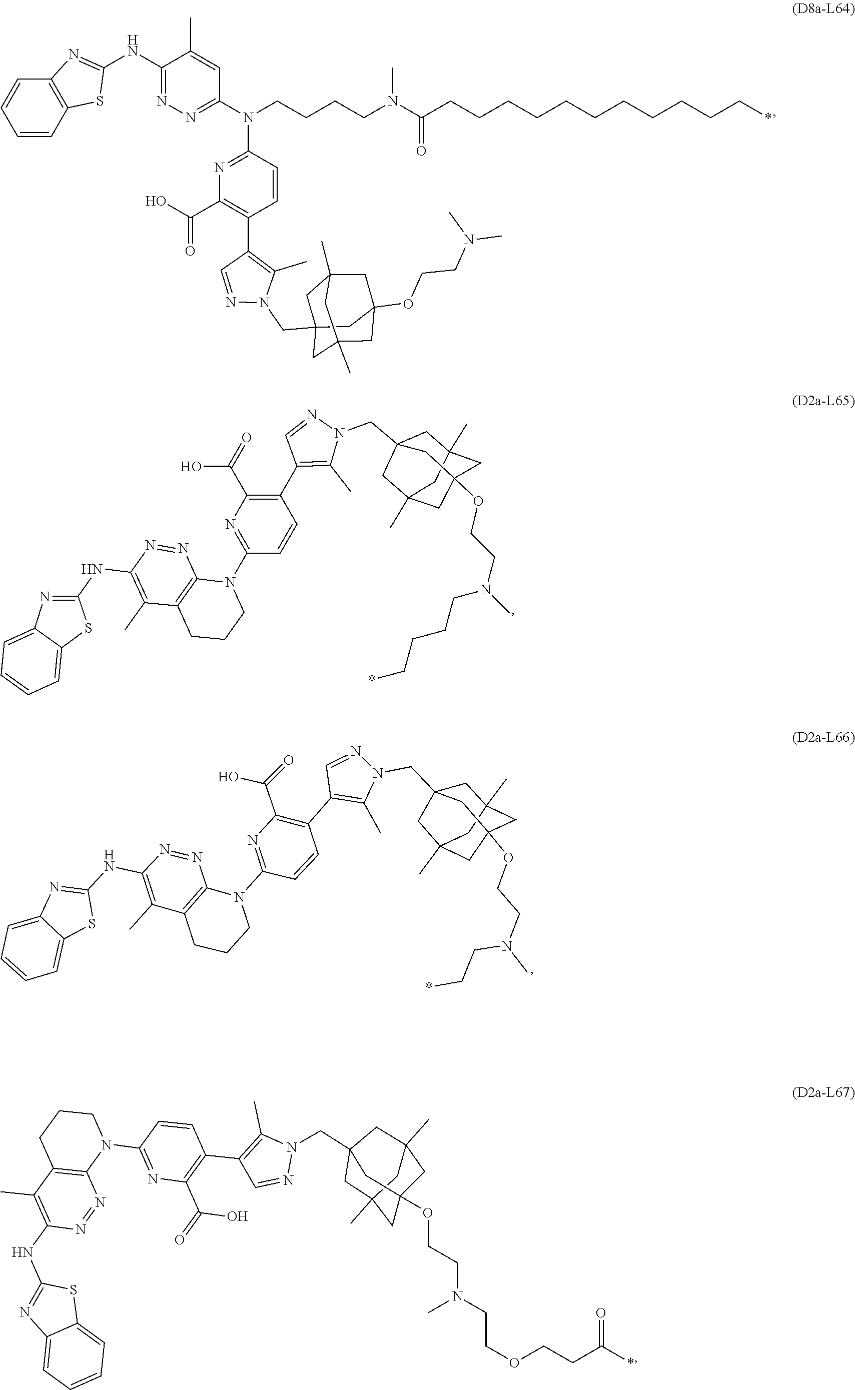

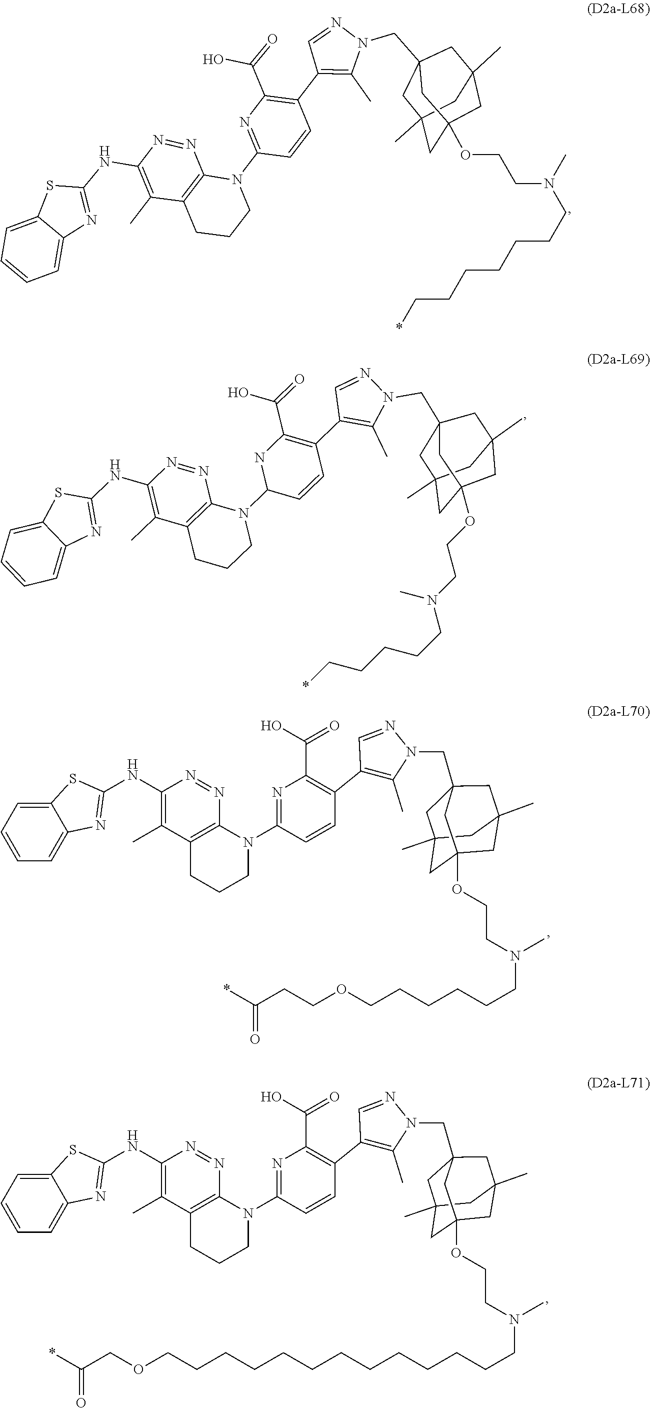

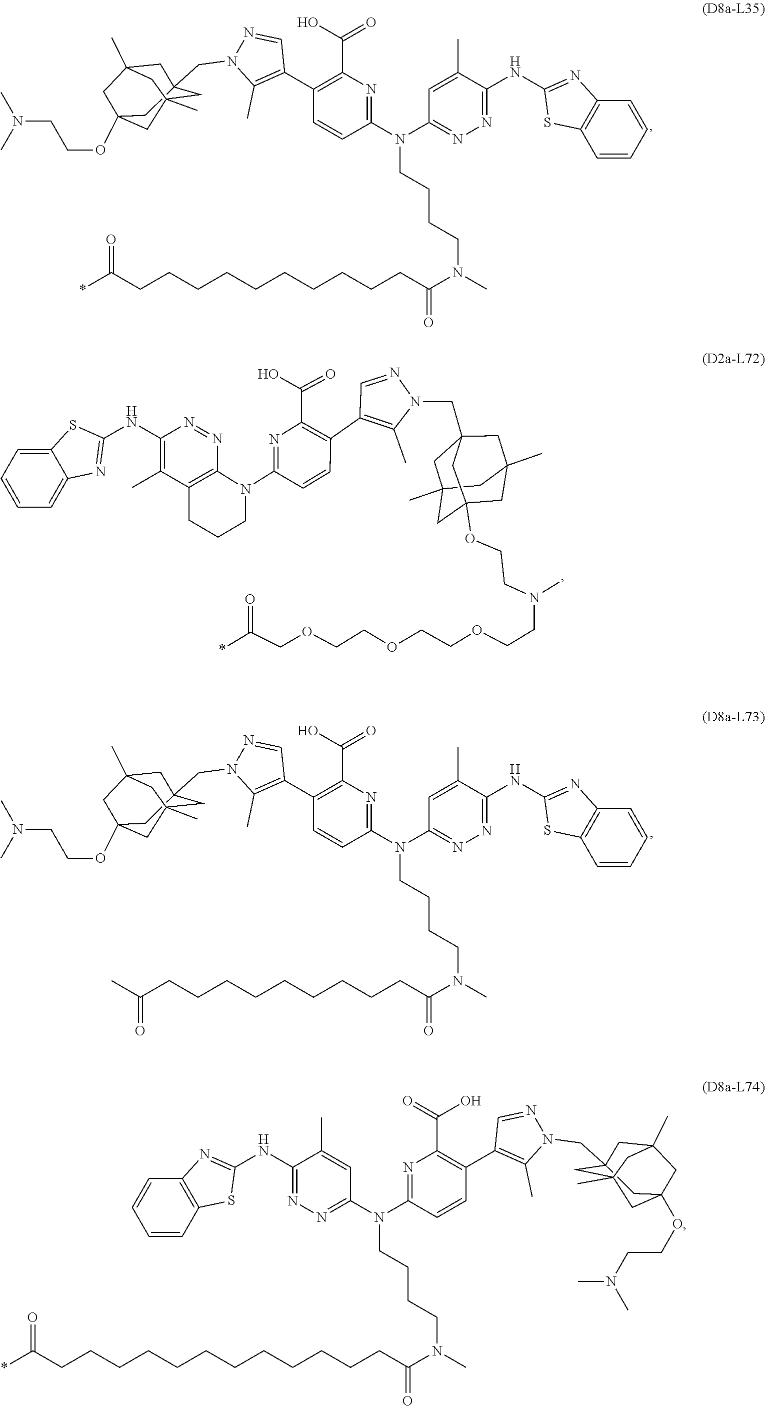

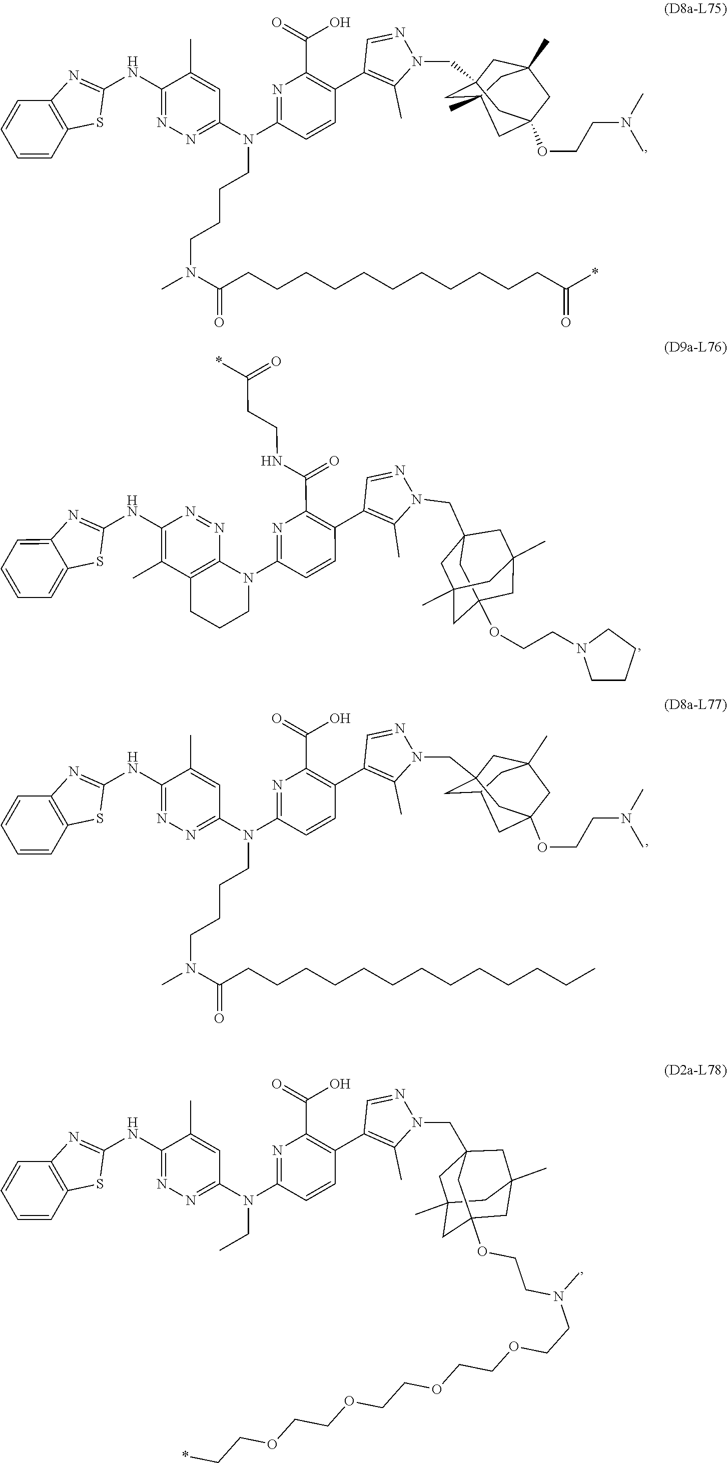

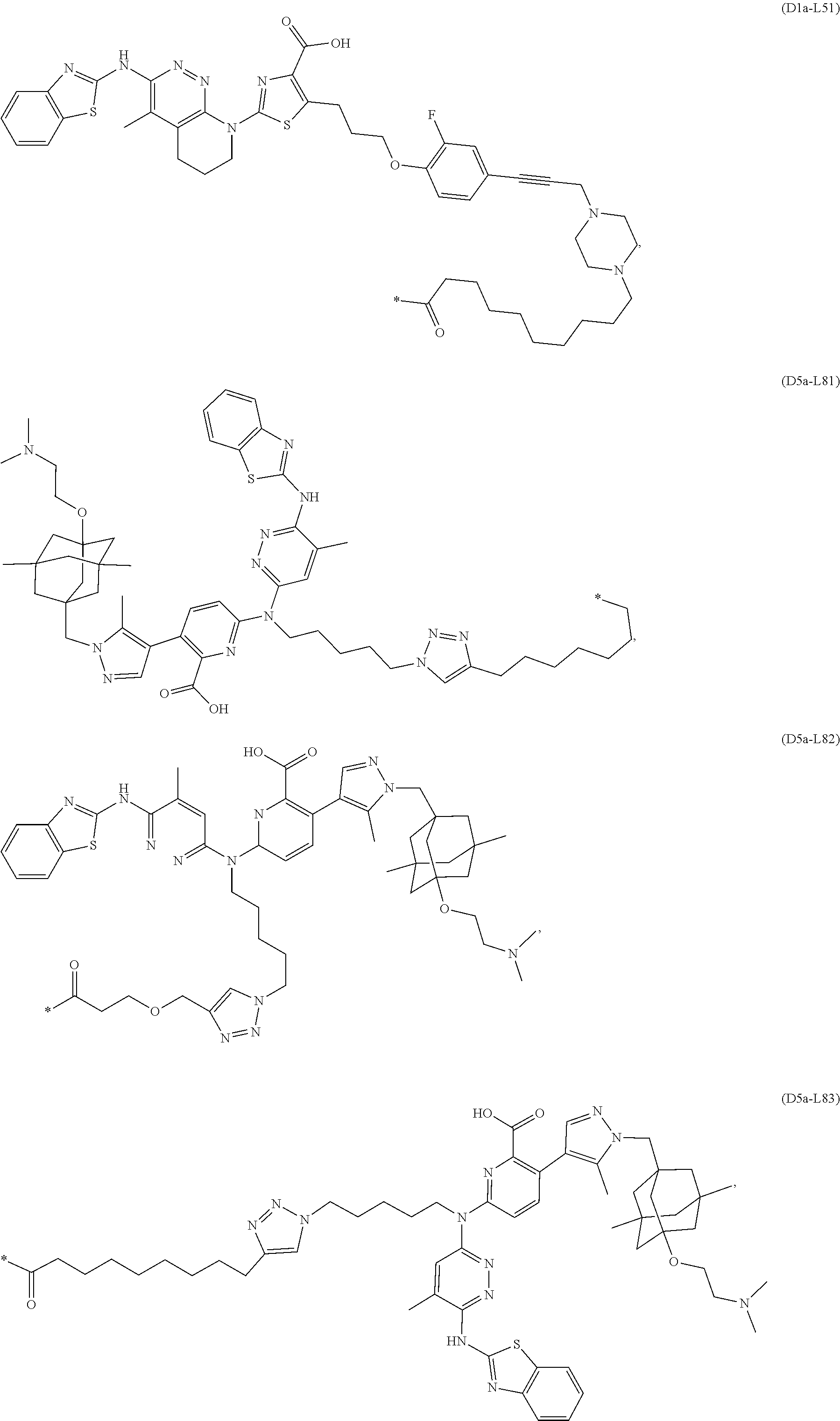

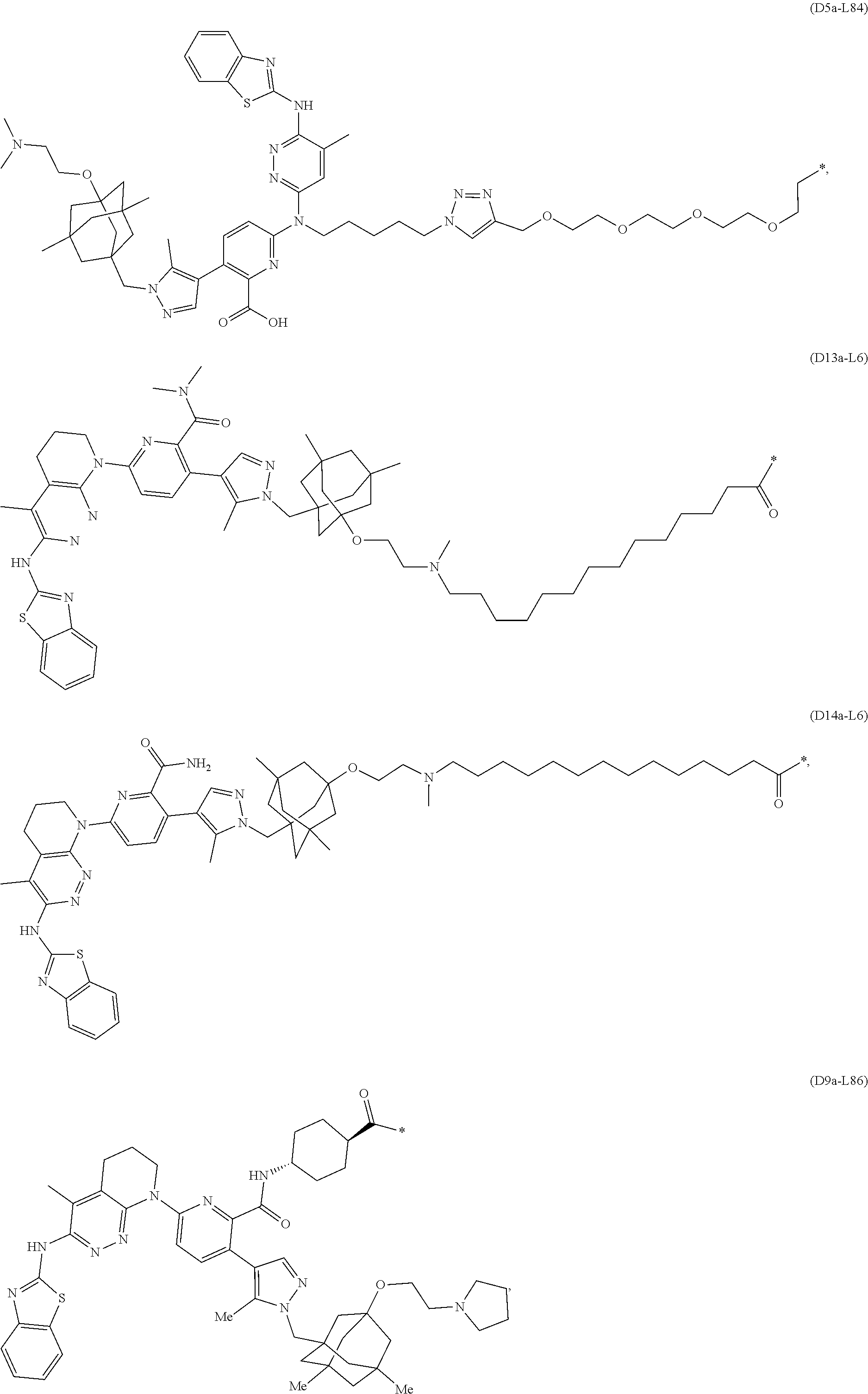

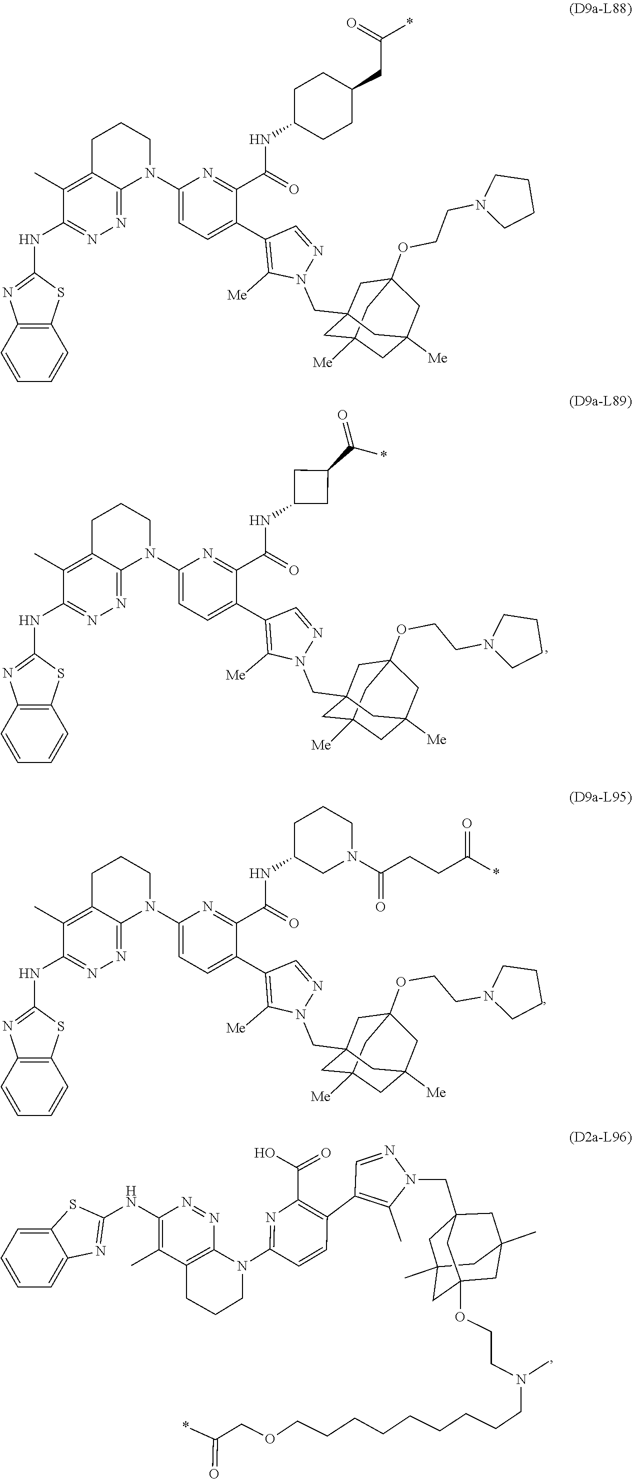 [SMILES: *C(=O)C[C@H]1CC[C@H](NC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)CC1 ; *C(=O)[C@H]1C[C@H](NC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C1 ; *C(=O)CCC(=O)N1CCC[C@@H](NC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C1 ; *C(=O)COCCCCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2]

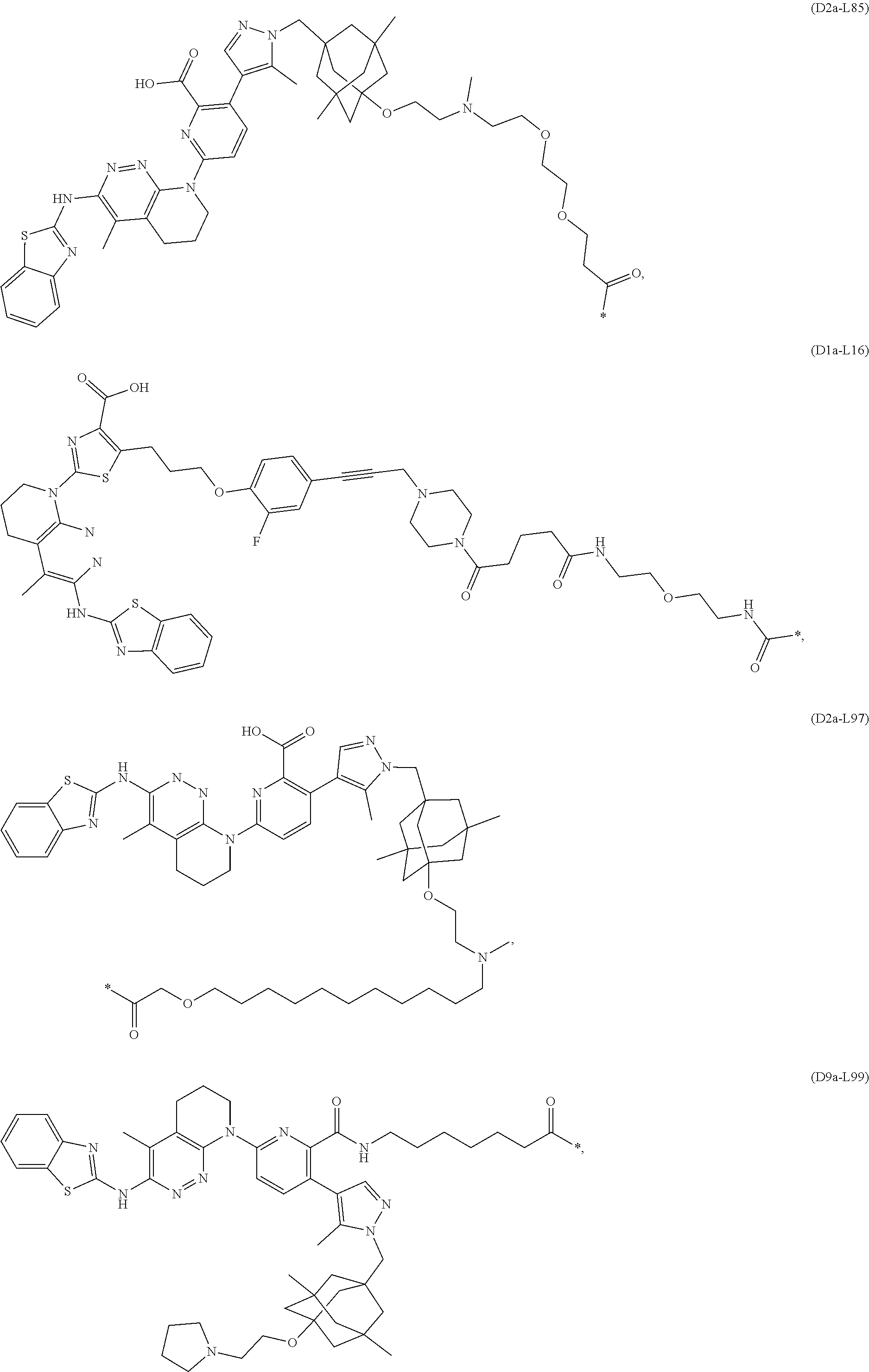

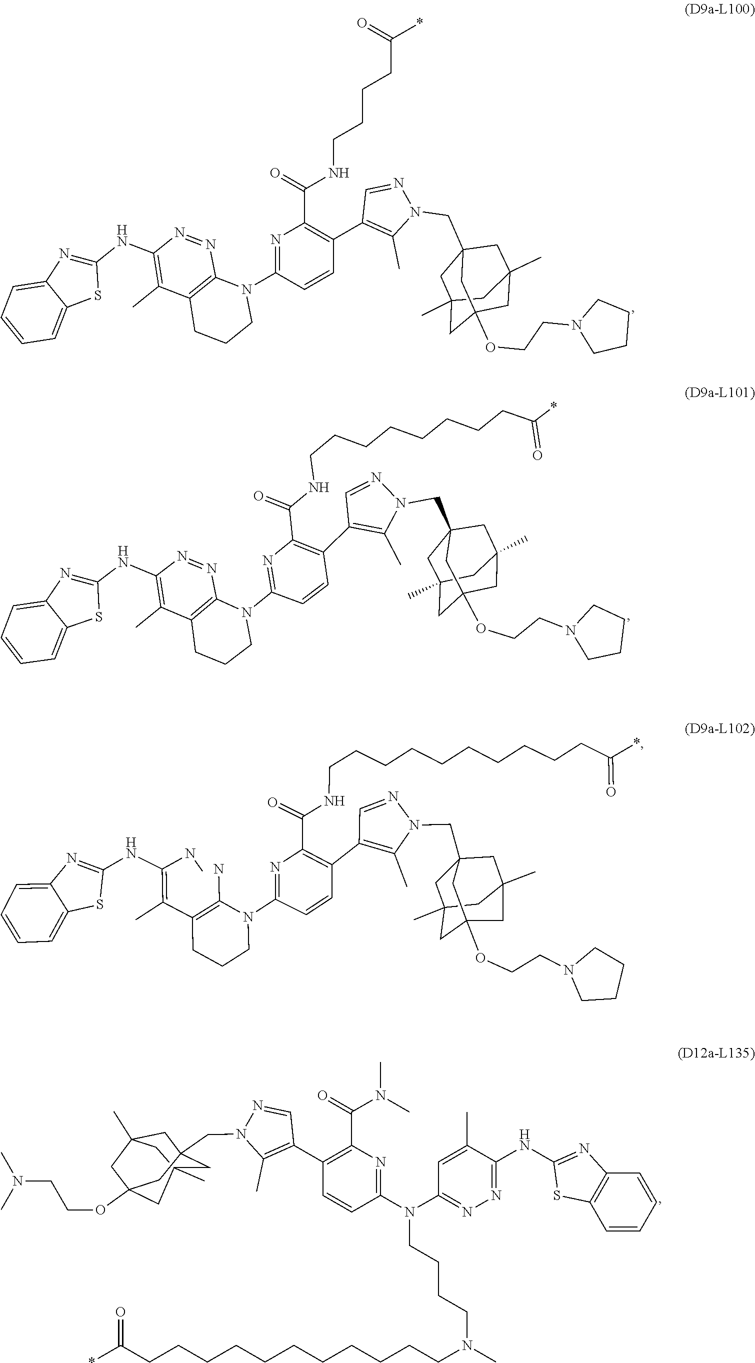

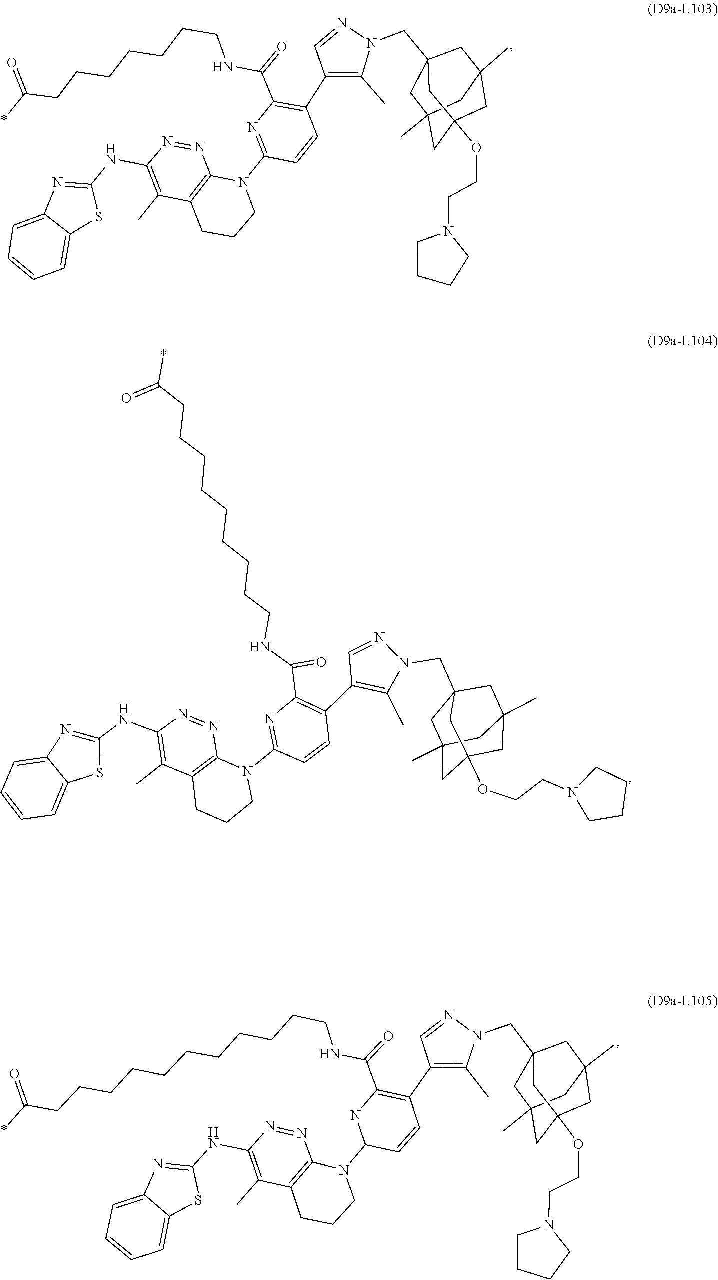

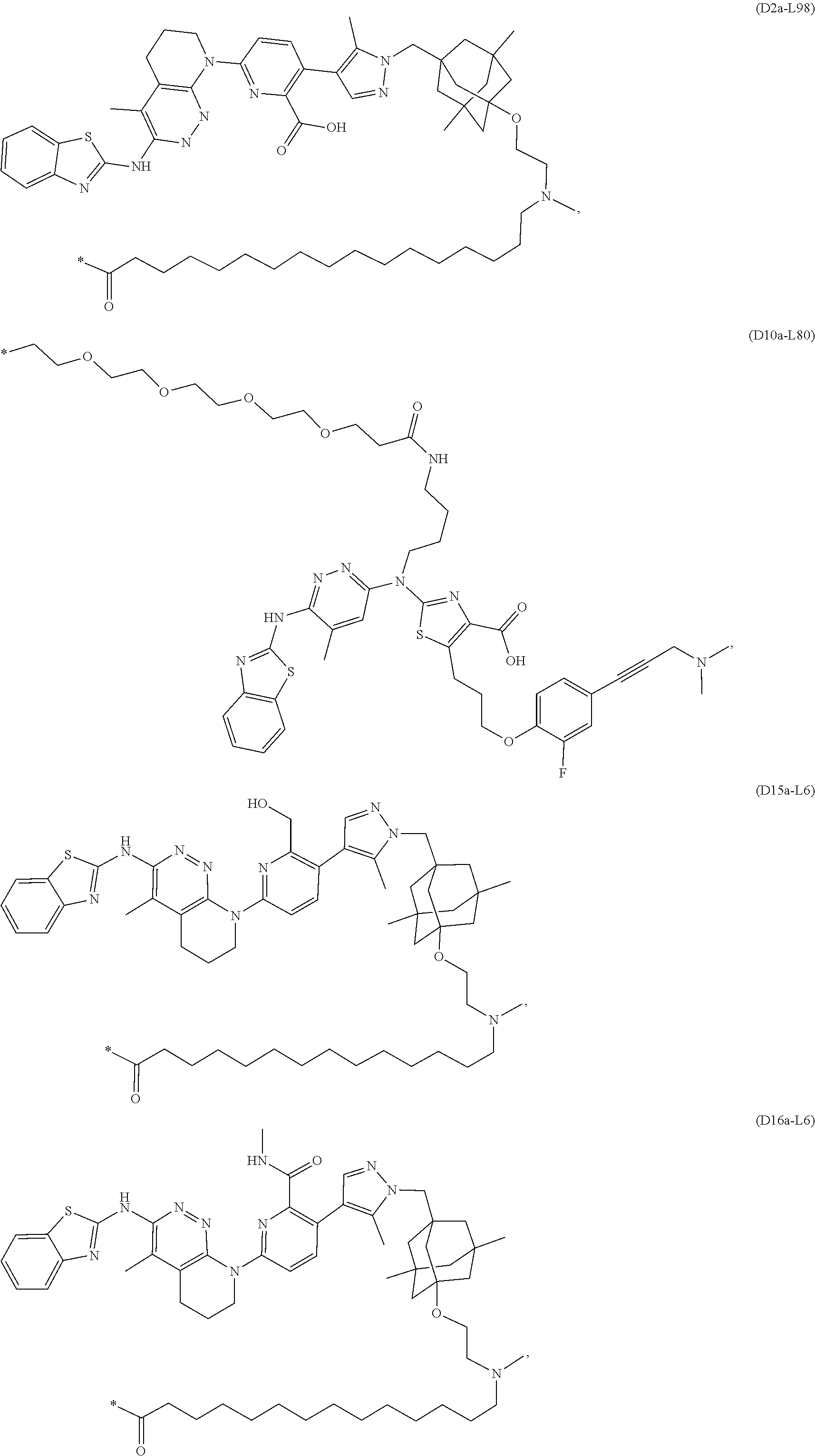

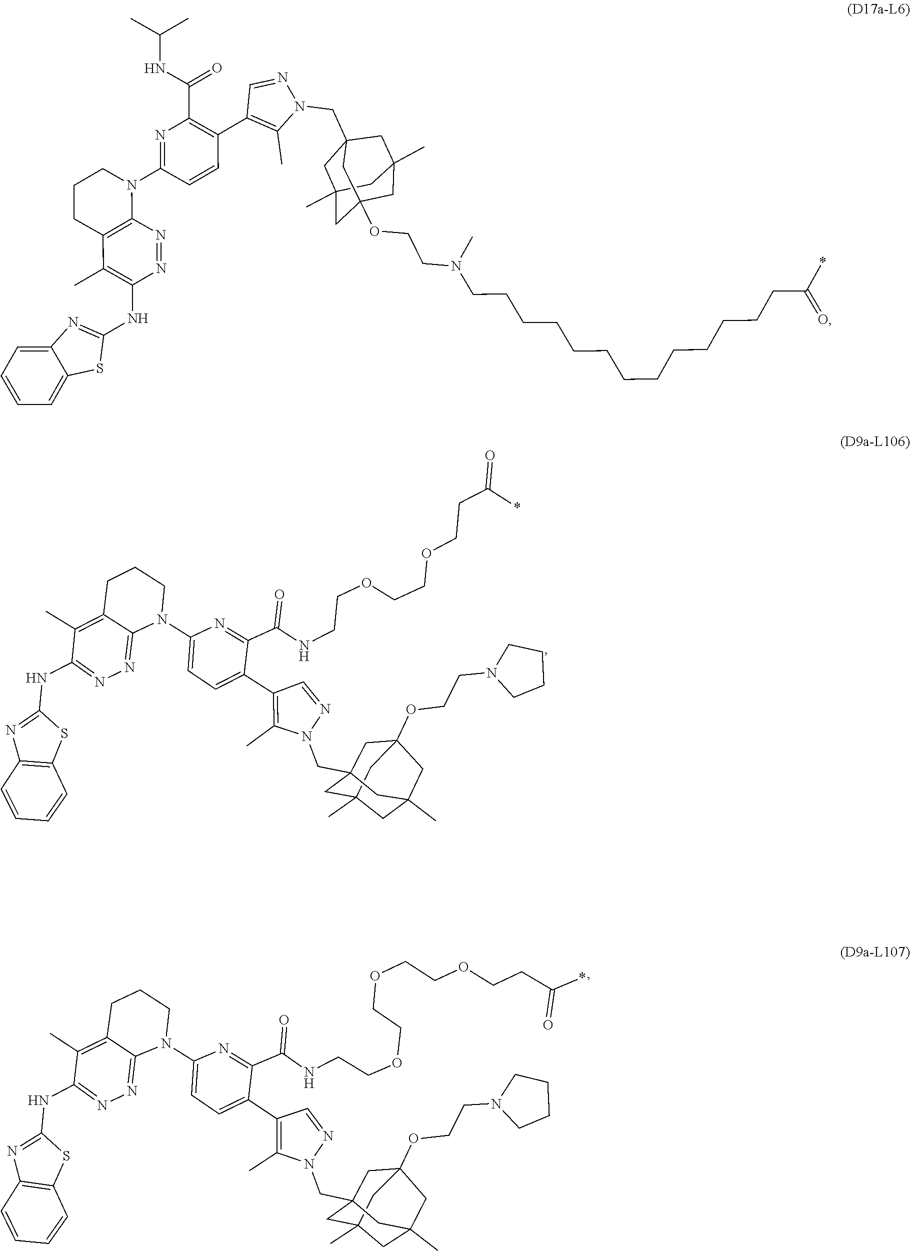 [SMILES: *C(=O)CCCCCCCCCCCCCN(C)CCOC12CC3(C)CC(C)(CC(Cn4ncc(-c5ccc(N6CCCc7c6nnc(Nc6nc8ccccc8s6)c7C)nc5C(=O)NC(C)C)c4C)(C3)C1)C2 ; *C(=O)CCOCCOCCNC(=O)c1nc(N2CCCc3c2nnc(Nc2nc4ccccc4s2)c3C)ccc1-c1cnn(CC23CC4(C)CC(C)(C2)CC(OCCN2CCCC2)(C4)C3)c1C ; *C(=O)CCOCCOCCOCCNC(=O)c1nc(N2CCCc3c2nnc(Nc2nc4ccccc4s2)c3C)ccc1-c1cnn(CC23CC4(C)CC(C)(C2)CC(OCCN2CCCC2)(C4)C3)c1C]

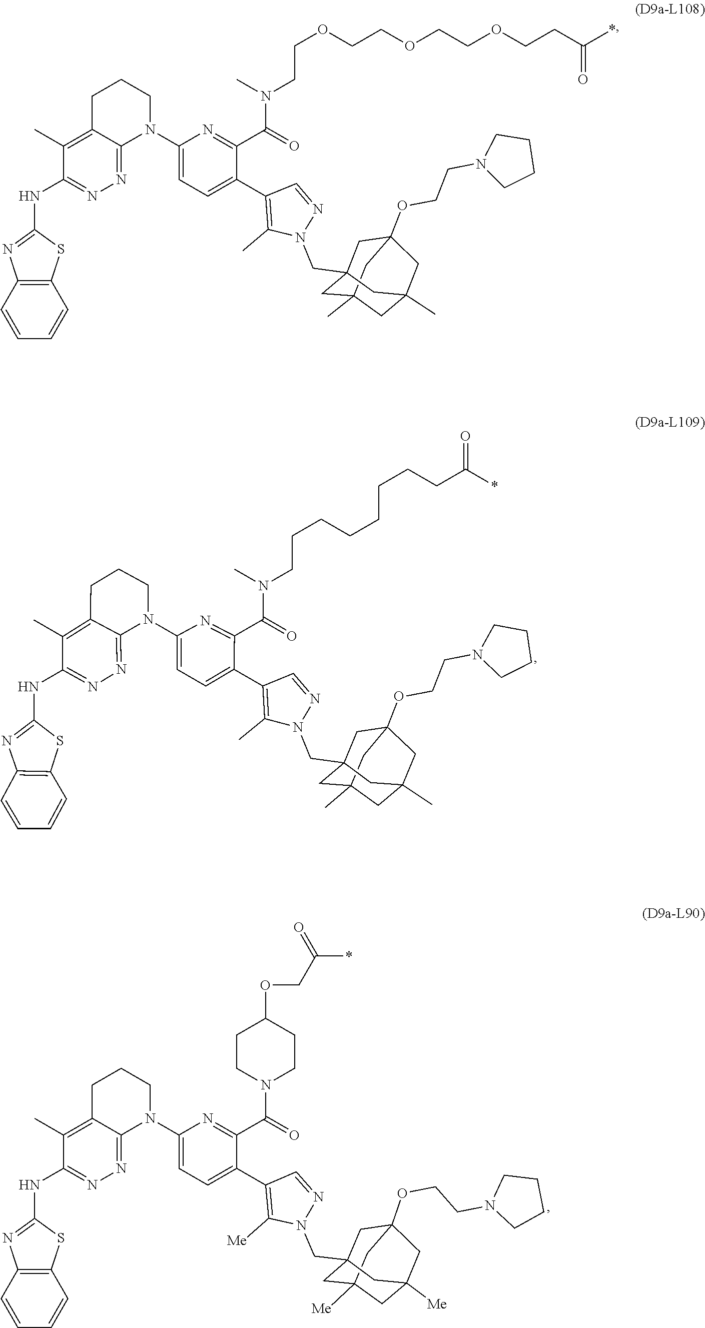 [SMILES: *C(=O)CCOCCOCCOCCN(C)C(=O)c1nc(N2CCCc3c2nnc(Nc2nc4ccccc4s2)c3C)ccc1-c1cnn(CC23CC4(C)CC(C)(C2)CC(OCCN2CCCC2)(C4)C3)c1C ; *C(=O)CCCCCCCCN(C)C(=O)c1nc(N2CCCc3c2nnc(Nc2nc4ccccc4s2)c3C)ccc1-c1cnn(CC23CC4(C)CC(C)(C2)CC(OCCN2CCCC2)(C4)C3)c1C ; *C(=O)COC1CCN(C(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)CC1]

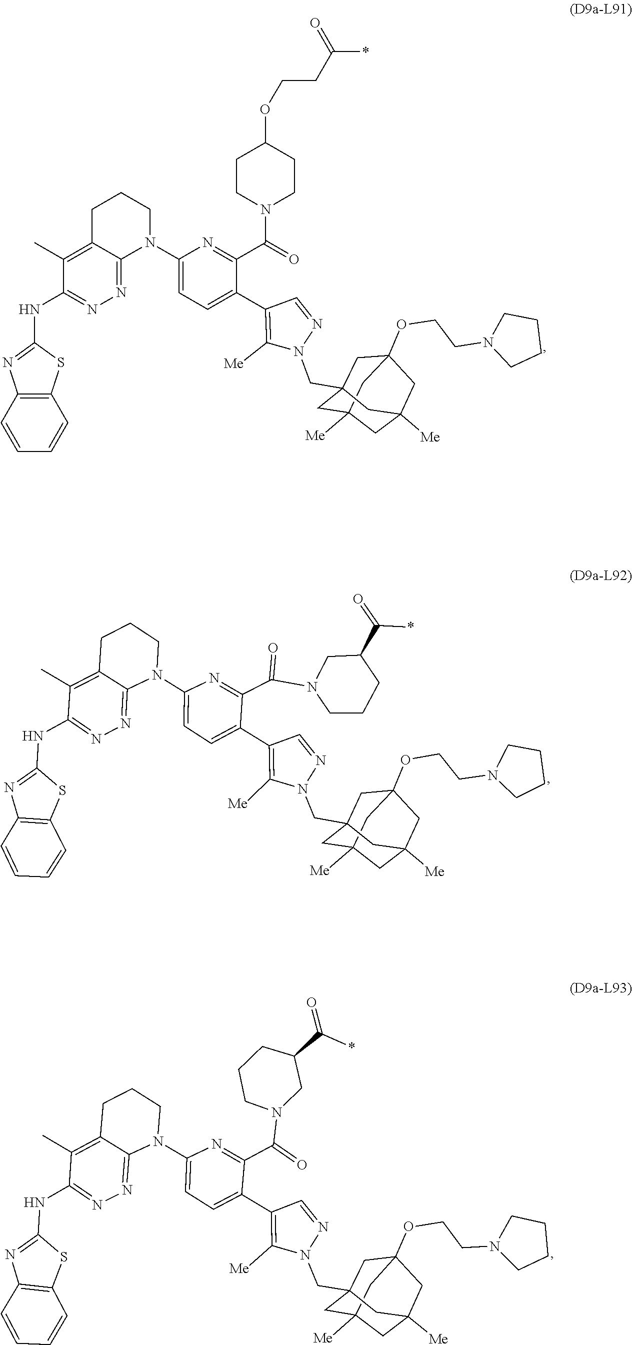

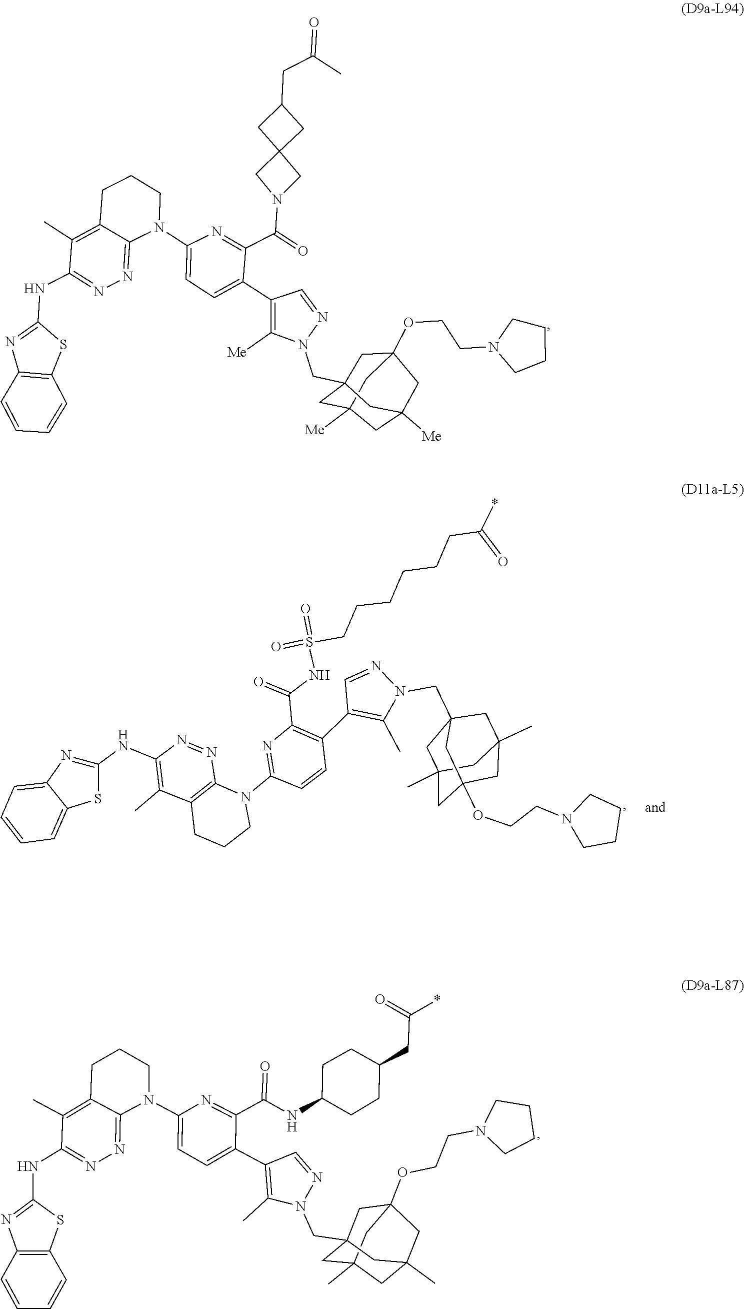 [SMILES: CC(=O)CC1CC2(C1)CN(C(=O)c1nc(N3CCCc4c3nnc(Nc3nc5ccccc5s3)c4C)ccc1-c1cnn(CC34CC5(C)CC(C)(C3)CC(OCCN3CCCC3)(C5)C4)c1C)C2 ; *C(=O)CCCCCCS(=O)(=O)NC(=O)c1nc(N2CCCc3c2nnc(Nc2nc4ccccc4s2)c3C)ccc1-c1cnn(CC23CC4(C)CC(C)(C2)CC(OCCN2CCCC2)(C4)C3)c1C ; *C(=O)C[C@H]1CC[C@@H](NC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)CC1]

wherein:
-* is a bond to the DSM;
and the remaining variable are as described in the first embodiment.

In a thirty-sixth embodiment of the present disclosure, for the compound of formula (A), or an enantiomer, a diastereomer, and/or a pharmaceutically acceptable salt thereof, DSM in Formula (A) is a E3 ligase recognition agent; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth or thirty-fifth embodiment.

In a thirty-seventh embodiment of the present disclosure, for the compound of formula (A), or the enantiomer, the diastereomer, and/or the pharmaceutically acceptable salt thereof, DSM in Formula (A) is a VHL ligand, a thalidomide cereblon binder or an inhibitor of apoptosis (IAP) E3 ligases; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth or thirty-fifth embodiment.

In a thirty-eighth embodiment of the present disclosure, for the compound of formula (A), or the enantiomer, the diastereomer, and/or the pharmaceutically acceptable salt thereof, DSM in Formula (A) represents any one of the following attached to L:

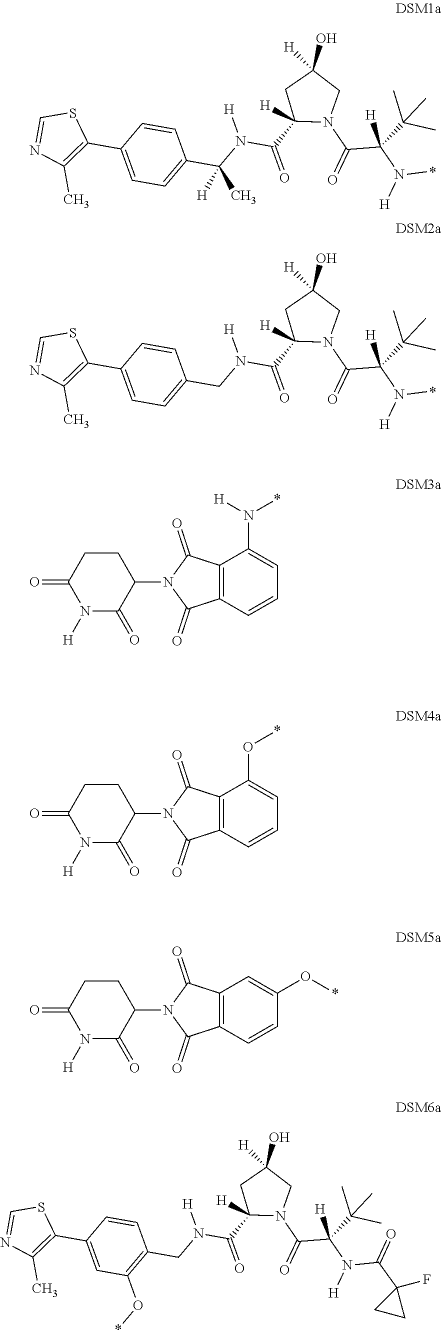 [SMILES: *N[C@H](C(=O)N1C[C@H](O)C[C@H]1C(=O)N[C@@H](C)c1ccc(-c2scnc2C)cc1)C(C)(C)C ; *N[C@H](C(=O)N1C[C@H](O)C[C@H]1C(=O)NCc1ccc(-c2scnc2C)cc1)C(C)(C)C ; *Nc1cccc2c1C(=O)N(C1CCC(=O)NC1=O)C2=O ; *Oc1cccc2c1C(=O)N(C1CCC(=O)NC1=O)C2=O ; *Oc1ccc2c(c1)C(=O)N(C1CCC(=O)NC1=O)C2=O ; *Oc1cc(-c2scnc2C)ccc1CNC(=O)[C@@H]1C[C@@H](O)CN1C(=O)[C@@H](NC(=O)C1(F)CC1)C(C)(C)C]

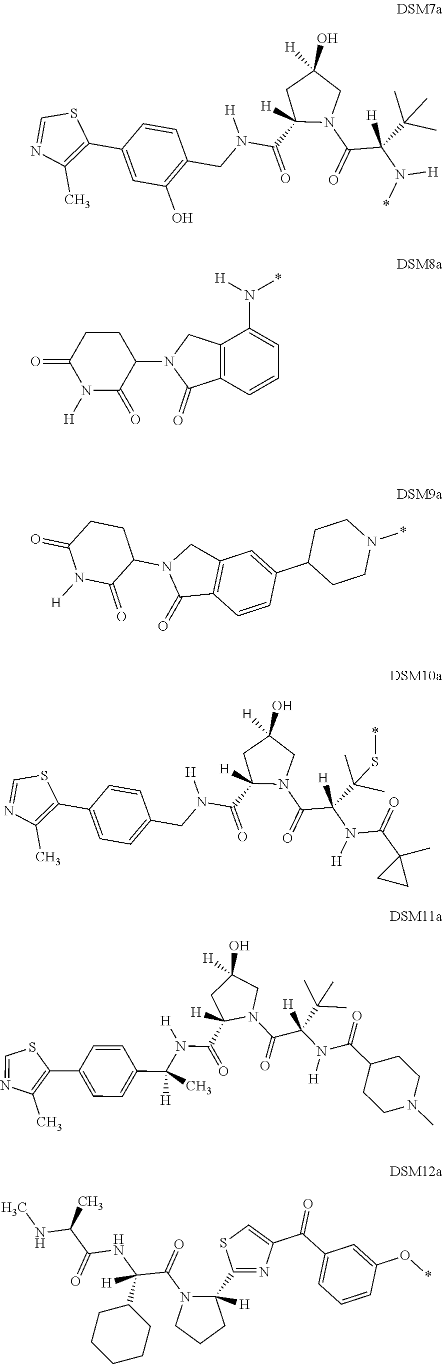

or an enantiomer, a diastereoisomer, and/or a pharmaceutically acceptable salt of any one of the foregoing, wherein:
-* represents a bond to the linker (L); and
the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth or thirty-fifth embodiment.

In a thirty-ninth embodiment of the present disclosure, for the compound of formula (A), or the enantiomer, the diastereomer, and/or the pharmaceutically acceptable salt thereof, DSM represents the following attached to L:

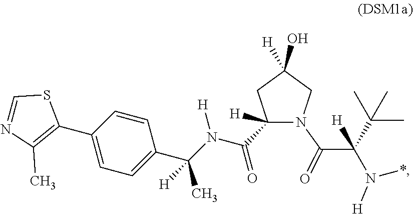

or an enantiomer, a diastereoisomer, and/or a pharmaceutically acceptable salt of any one of the foregoing, wherein:
-* represents a bond to the linker (L); and
the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth or thirty-fifth embodiment.

In a fortieth embodiment, the compound of the present disclosure is any one of the compounds in Table 7, or an enantiomer, a diastereomer, and/or a pharmaceutically acceptable salt thereof.

The present disclosure also provides pharmaceutical compositions comprising a PROTAC compounds describe herein (e.g., the compound of the first to twenty-eighth embodiments described above) and a pharmaceutically acceptable carrier.

The present disclosure also relates to a method of treating a subject having or suspected of having a cancer comprises administering to the subject a therapeutically effective amount of a compound described herein (e.g., the compound of the first to twenty-eighth embodiments described above) or a pharmaceutical composition thereof.

In some embodiments, the cancer is a solid tumor or a hematological cancer.

In some embodiments, the cancer is a breast cancer, multiple myeloma, plasma cell myeloma, leukemia, lymphoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, chronic lymphocytic leukemia, prostate cancer, small cell lung cancer, or spleen cancer.

In some embodiments, the PROTAC compound is administered as monotherapy.

In some embodiments, the PROTAC compound is administered adjunctive to another therapeutic agent or radiation therapy.

In some embodiments, the PROTAC compound is administered in an amount effective to sensitize the tumor cells to one or more additional therapeutic agents and/or radiation therapy.

In some embodiments, the methods described above further comprise administering to the subject in need thereof at least one additional therapeutic agent.

In some embodiments, the additional therapeutic agent is a Bcl-2 inhibitor, a taxane, a MEK inhibitor, an ERK inhibitor, or a RAF inhibitor.

Also included in the present disclosure is a PROTAC compound of Formula (A) for use in a method described above (e.g., a method of treating a subject having or suspected of having a cancer). The present disclosure also relates to the use of a PROTAC compound of Formula (A) for the manufacture of a medicament for treating a subject having or suspected of having a cancer.

### DETAILED DESCRIPTION

The disclosed compositions and methods may be understood more readily by reference to the following detailed description.

Throughout this text, the descriptions refer to compositions and methods of using the compositions. Where the disclosure describes or claims a feature or embodiment associated with a composition, such a feature or embodiment is equally applicable to the methods of using the composition. Likewise, where the disclosure describes or claims a feature or embodiment associated with a method of using a composition, such a feature or embodiment is equally applicable to the composition.

When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise. All references cited herein are incorporated by reference for any purpose. Where a reference and the specification conflict, the specification will control.

Unless the context of a description indicates otherwise, e.g., in the absence of symbols indicating specific point(s) of connectivity, when a structure or fragment of a structure is drawn, it may be used on its own or attached to other components of a compound, and it may do so with any orientation, e.g., with the DSM (degradation signaling moiety) attached at any suitable attachment point to a chemical moiety such as a linker. Where indicated, however, components of the PROTAC compounds described herein are attached in the orientation shown in a given formula.

It is to be appreciated that certain features of the disclosed compositions and methods, which are, for clarity, described herein in the context of separate embodiments, may also be provided in combination in a single embodiment. Conversely, various features of the disclosed compositions and methods that are, for brevity, described in the context of a single embodiment, may also be provided separately or in any sub-combination.

As used throughout this application, PROTAC compounds can be identified using a naming convention in the general format of “DSM-linker-Bcl-xL inhibitor compound.” For example only, if a compound is referred to as “DSM1a-L1-D1a”, such a compound would comprise a DSM designated as DSM1a, a linker designated as L1, and a Bcl-xL inhibitor compound moiety designated as D1a. Similar designation can be used to identify components or moieties in the PROTAC compounds described herein.

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of this disclosure include, for example, isotopes of hydrogen, carbon, nitrogen, oxygen, fluorine, and chlorine, such as ³H, ¹¹C, ¹³C, ¹⁴C ¹⁵N, ¹⁸F, and ³⁶Cl. Accordingly, it should be understood that the present disclosure includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an 18F or labeled compound may be particularly desirable for PET or SPECT studies. Isotopically-labeled compounds can generally be prepared by conventional techniques known to those skilled in the art, e.g., using an appropriate isotopically-labeled reagents in place of the non-labeled reagent previously employed.

### Definitions

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include plural forms unless the context clearly dictates otherwise. The terms “comprising”, “having”, “being of” as in “being of a chemical formula”, “including”, and “containing” are to be construed as open terms (i.e., meaning “including but not limited to”) unless otherwise noted. Additionally whenever “comprising” or another open-ended term is used in an embodiment, it is to be understood that the same embodiment can be more narrowly claimed using the intermediate term “consisting essentially of” or the closed term “consisting of”.

The term “alkyl”, as used herein, refers to a straight or branched hydrocarbon chain radical consisting solely of carbon and hydrogen atoms, containing no unsaturation. The term “C₁-C₆alkyl”, as used herein, refers to a straight or branched hydrocarbon chain radical consisting solely of carbon and hydrogen atoms, containing no unsaturation, having from one to six carbon atoms, and which is attached to the rest of the molecule by a single bond. Non-limiting examples of “C₁-C₆alkyl” groups include methyl (a C₁alkyl), ethyl (a C₂alkyl), 1-methylethyl (a C₆alkyl), n-propyl (a C₆alkyl), isopropyl (a C₆alkyl), n-butyl (a C₄alkyl), isobutyl (a C₄alkyl), sec-butyl (a C₄alkyl), tert-butyl (a C₄alkyl), n-pentyl (a C₆alkyl), isopentyl (a C₆alkyl), neopentyl (a C₆alkyl) and hexyl (a C₆alkyl).

The term “alkenyl”, as used herein, refers to a straight or branched hydrocarbon chain radical group consisting solely of carbon and hydrogen atoms, containing at least one double bond. The term “C₂-C₆alkenyl”, as used herein, refers to a straight or branched hydrocarbon chain radical group consisting solely of carbon and hydrogen atoms, containing at least one double bond, having from two to six carbon atoms, which is attached to the rest of the molecule by a single bond. Non-limiting examples of “C₂-C₆alkenyl” groups include ethenyl (a C₂alkenyl), prop-1-enyl (a C₃alkenyl), but-1-enyl (a C₄alkenyl), pent-1-enyl (a C₅alkenyl), pent-4-enyl (a C₅alkenyl), penta-1,4-dienyl (a C₅alkenyl), hexa-1-enyl (a C₆alkenyl), hexa-2-enyl (a C₆alkenyl), hexa-3-enyl (a C₆alkenyl), hexa-1-,4-dienyl (a C₆alkenyl), hexa-1-,5-dienyl (a C₆alkenyl) and hexa-2-,4-dienyl (a C₆alkenyl). The term “C₂-C₃alkenyl”, as used herein, refers to a straight or branched hydrocarbon chain radical group consisting solely of carbon and hydrogen atoms, containing at least one double bond, having from two to three carbon atoms, which is attached to the rest of the molecule by a single bond. Non-limiting examples of “C₂-C₃alkenyl” groups include ethenyl (a C₂alkenyl) and prop-1-enyl (a C₃alkenyl).

The term “alkylene”, as used herein, refers to a bivalent straight or branched hydrocarbon chain radical consisting solely of carbon and hydrogen atoms and containing no unsaturation. The term “C₁-C₆alkylene”, as used herein, refers to a bivalent straight or branched hydrocarbon chain radical consisting solely of carbon and hydrogen atoms, containing no unsaturation, having from one to six carbon atoms. Non-limiting examples of “C₁-C₆alkylene” groups include methylene (a C₁alkylene), ethylene (a C₂alkylene), 1-methylethylene (a C₃alkylene), n-propylene (a C₃alkylene), isopropylene (a C₃alkylene), n-butylene (a C₄alkylene), isobutylene (a C₄alkylene), sec-butylene (a C₄alkylene), tert-butylene (a C₄alkylene), n-pentylene (a C₅alkylene), isopentylene (a C₆alkylene), neopentylene (a C₅alkylene), and hexylene (a C₆alkylene).

The term “alkenylene”, as used herein, refers to a bivalent straight or branched hydrocarbon chain radical consisting solely of carbon and hydrogen atoms and containing at least one double bond. The term “C₂-C₆alkenylene”, as used herein, refers to a bivalent straight or branched hydrocarbon chain radical group consisting solely of carbon and hydrogen atoms, containing at least one double bond, and having from two to six carbon atoms. Non-limiting examples of “C₂-C₆alkenylene” groups include ethenylene (a C₂alkenylene), prop-1-enylene (a C₃alkenylene), but-1-enylene (a C₄alkenylene), pent-1-enylene (a C₅alkenylene), pent-4-enylene (a C₅alkenylene), penta-1,4-dienylene (a C₅alkenylene), hexa-1-enylene (a C₆alkenylene), hexa-2-enylene (a C₆alkenylene), hexa-3-enylene (a C₆alkenylene), hexa-1-,4-dienylene (a C₆alkenylene), hexa-1-,5-dienylene (a C₆alkenylene) and hexa-2-,4-dienylene (a C₆alkenylene). The term “C₂-C₆alkenylene”, as used herein, refers to a bivalent straight or branched hydrocarbon chain radical group consisting solely of carbon and hydrogen atoms, containing at least one double bond, and having from two to thee carbon atoms. Non-limiting examples of “C₂-C₃alkenylene” groups include ethenylene (a C₂alkenylene) and prop-1-enylene (a C₃alkenylene).

The term “aryl” as used herein, refers to a phenyl, naphthyl, biphenyl or indenyl group.

The term “cycloalkyl” as used herein, refers to any mono- or bi-cyclic non-aromatic carbocyclic group containing from 3 to 10 ring members, which may include fused, bridged or spiro ring systems. Non-limiting examples of fused bicyclic or bridged polycyclic ring systems include bicyclo[1.1.1]pentane, bicyclo[2.1.1]hexane, bicyclo[2.2.1]heptane, bicyclo[3.1.1]heptane, bicyclo[3.2.1]octane, bicyclo[2.2.2]octane and adamantanyl. Non-limiting examples monocyclic C₃-C₈cycloalkyl groups include cyclopropyl, cyclobutyl, cyclopentyl, cyclohexyl, cycloheptyl and cyclooctyl groups.

The term “cycloalkylene” refers to a cycloalkyl, as defined herein, having two monovalent radical centers derived by the removal of two hydrogen atoms from the same or two different carbon atoms of a parent cycloalkyl. Examples of cycloalkylene include, but are not limited to, cyclopropylene, cyclobutylene, cyclopentylene and cyclohexylene. Cycloalkylenes of the present disclosure include monocyclic, bicyclic and tricyclic ring structures.

The term “haloalkyl,” as used herein, refers to a linear or branched alkyl chain substituted with one or more halogen groups in place of hydrogens along the hydrocarbon chain. Examples of halogen groups suitable for substitution in the haloalkyl group include Fluorine, Bromine, Chlorine, and Iodine. Haloalkyl groups may include substitution with multiple halogen groups in place of hydrogens in an alkyl chain, wherein said halogen groups can be attached to the same carbon or to another carbon in the alkyl chain.

The term “heteroaryl” as used herein, refers any mono- or bi-cyclic group composed of from 5 to 10 ring members, having at least one aromatic moiety and containing from 1 to 4 hetero atoms selected from oxygen, sulphur and nitrogen (including quaternary nitrogens).

The term “heterocycloalkyl” means any mono- or bi-cyclic non-aromatic carbocyclic group, composed of from 3 to 10 ring members, and containing from one to 3 hetero atoms selected from oxygen, sulphur, SO, SO₂ and nitrogen, it being understood that bicyclic group may be fused or spiro type. C₃-C₆heterocycloalkyl refers to heterocycloalkyl having 3 to 8 ring carbon atoms. The heterocycloalkyl can have 4 to 10 ring members.

The terms “heteroarylene” and “heterocycloalkylene” mean divalent heteroaryl and heterocycloalkyl groups, including heterocyclic groups with bicyclic and tricyclic ring structures.

As used herein, the alkyl, alkenyl, alkynyl, alkoxy, amino, aryl, heteroaryl, cycloalkyl, and heterocycloalkyl groups may be optionally substituted by 1 to 4 groups selected from optionally substituted linear or branched (C₁-C₆)alkyl, optionally substituted linear or branched (C₂-C₆)alkenyl group, optionally substituted linear or branched (C₂-C₆)alkynyl group, optionally substituted linear or branched (C₁-C₆)alkoxy, optionally substituted (C₁-C₆)alkyl-S—, hydroxy, oxo (or N-oxide where appropriate), nitro, cyano, —C(O)—OR₀′, —O—C(O)—R₀′, —C(O)—NR₀′R₀″, —NR₀′R₀″, —(═NR₀′)—OR₀″, linear or branched (C₁-C₆) haloalkyl, trifluoromethoxy, or halogen, wherein R₀′ and R₀″ are each independently a hydrogen atom or an optionally substituted linear or branched (C₁-C₆)alkyl group, and wherein one or more of the carbon atoms of linear or branched (C₁-C₆)alkyl group is optionally deuterated.

The term “linker”, as used herein, refers to a chemical moiety in Formula (A) that connects D to DSM.

The term “polyoxyethylene”, “polyethylene glycol” or “PEG”, as used herein, refers to a linear chain, a branched chain or a star shaped configuration comprised of (OCH₂CH₂) groups. In certain embodiments a polyethylene or PEG group is —(OCH₂CH₂)ₜ*—, where t is 1-40 or 4-40, and where the “-” indicates the end directed toward the self-immolative spacer and the “*-” indicates the point of attachment to a terminal end group R′ where R′ is OH, OCH₃ or OCH₂CH₂C(═O)OH. In other embodiments a polyethylene or PEG group is —(CH₂CH₂O)ₜ*—, where t is 1-40 or 4-40, and where the “-” indicates the end directed toward the self-immolative spacer and the “*-” indicates the point of attachment to a terminal end group R″ where R″ is H, CH₃ or CH₂CH₂C(═O)OH. For example, the term “PEG12” as used herein means that t is 12.

The term “polyalkylene glycol”, as used herein, refers to a linear chain, a branched chain or a star shaped configuration comprised of (O(CH₂)ₘ)ₙ groups. In certain embodiments a polyethylene or PEG group is —(O(CH₂)ₘ)ₜ*—, where m is 1-10, t is 1-40 or 4-40, and where the “-” indicates the end directed toward the self-immolative spacer and the “*-” indicates the point of attachment to a terminal end group R′ where R′ is OH, OCH₃ or OCH₂CH₂C(═O)OH. In other embodiments a polyethylene or PEG group is —((CH₂)ₘO)ₜ*—, where m is 1-10, t is 1-40 or 4-40, and where the “-” indicates the end directed toward the self-immolative spacer and the “*-” indicates the point of attachment to a terminal end group R″ where R″ is H, CH₃ or CH₂CH₂C(═O)OH.

The term “about” or “approximately,” when used in the context of numerical values and ranges, refers to values or ranges that approximate or are close to the recited values or ranges such that the embodiment may perform as intended, as is apparent to the skilled person from the teachings contained herein. In some embodiments, about means plus or minus 20%, 15%, 10%, 5%, 1%, 0.5%, or 0.1% of a numerical amount. In one embodiment, the term “about” refers to a range of values which are 10% more or less than the specified value. In another embodiment, the term “about” refers to a range of values which are 5% more or less than the specified value. In another embodiment, the term “about” refers to a range of values which are 1% more or less than the specified value.

The term “agent” is used herein to refer to a chemical compound, a mixture of chemical compounds, a biological macromolecule, an extract made from biological materials, or a combination of two or more thereof. The term “therapeutic agent” or “drug” refers to an agent that is capable of modulating a biological process and/or has biological activity. The Bcl-xL inhibitors and the PROTAC compounds comprising them, as described herein, are exemplary therapeutic agents.

The term “chemotherapeutic agent” or “anti-cancer agent” is used herein to refer to all agents that are effective in treating cancer (regardless of mechanism of action). Inhibition of metastasis or angiogenesis is frequently a property of a chemotherapeutic agent. Chemotherapeutic agents include antibodies, biological molecules, and small molecules, and encompass the Bcl-xL inhibitors and DSM conjugates comprising them, as described herein. A chemotherapeutic agent may be a cytotoxic or cytostatic agent. The term “cytostatic agent” refers to an agent that inhibits or suppresses cell growth and/or multiplication of cells. The term “cytotoxic agent” refers to a substance that causes cell death primarily by interfering with a cell's expression activity and/or functioning.

The terms “PROTAC conjugate,” “PROTAC compounds,” “PROTAC degraders,” “DSM-drug conjugate,” “DSM conjugate,” “Bcl-degrading conjugate,” “Bcl-xL degrader compounds,” “bifunctional Bcl-xL degrader compounds,” and “compound” are used interchangeably, and refer to one or more therapeutic compounds (e.g., a Bcl-xL inhibitor) that is covalently linked to a DSM such as an E3 ubiquitin ligase recruitment ligand. In some embodiments, the PROTAC compound is defined by the generic formula: D-L-DSM (Formula (A)), wherein DSM=a degradation signaling moiety, L=a linker moiety, and D=a drug moiety (e.g., a Bcl-xL inhibitor drug moiety).

The terms “degradation signaling moiety” and “DSM” are used herein to refer to degradation signaling compounds or moieties derived therefrom that induce degradation of targeting proteins, such as Bcl-xL. DSMs of this disclosure degrade targeted proteins by binding or recruiting at least one degradation protein, which is usually associated with the proteasome, the ubiquitin-proteasome pathways, or lysosomal proteolysis. DSMs of this disclosure include, but are not limited to, E3 ligase recognition or recruitment ligand.

The term “ubiquitin ligase” refers to a family of proteins that facilitate the transfer of ubiquitin to a specific substrate protein, targeting the substrate protein for degradation. Cereblon, for example, is an E3 Ubiquitin Ligase protein that alone or in combination with an E2 ubiquitin-conjugating enzyme causes the attachment of ubiquitin to a lysine on a target protein, and subsequently targets the specific protein substrate for degradation by the proteasome.

The term “B-cell lymphoma-extra large” or “Bcl-xL,” as used herein, refers to any native form of human Bcl-xL, an anti-apoptotic member of the Bcl-2 protein family. The term encompasses full-length human Bcl-xL (e.g., UniProt Reference Sequence: 007817-1; SEQ ID NO:71), as well as any form of human Bcl-xL that may result from cellular processing. The term also encompasses functional variants or fragments of human Bcl-xL, including but not limited to splice variants, allelic variants, and isoforms that retain one or more biologic functions of human Bcl-xL (i.e., variants and fragments are encompassed unless the context indicates that the term is used to refer to the wild-type protein only). Bcl-xL can be isolated from human, or may be produced recombinantly or by synthetic methods.

The term “inhibit” or “inhibition” or “inhibiting,” as used herein, means to reduce a biological activity or process by a measurable amount, and can include but does not require complete prevention or inhibition. In some embodiments, “inhibition” means to reduce the expression and/or activity of Bcl-xL and/or one or more upstream modulators or downstream targets thereof.

The term “Bcl-xL inhibitor,” as used herein, refers to an agent capable of reducing the expression and/or activity of Bcl-xL and/or one or more upstream modulators or downstream targets thereof. Exemplary Bcl-xL modulators (including exemplary inhibitors of Bcl-xL) are described in WO2010/080503, WO2010/080478, WO2013/055897, WO2013/055895, WO2016/094509, WO2016/094517, WO2016/094505, Tao et al., ACS Medicinal Chemistry Letters (2014), 5(10), 1088-109, and Wang et al., ACS Medicinal Chemistry Letters (2020), 11(10), 1829-1836, each of which are incorporated herein by reference as exemplary Bcl-xL modulators, including exemplary Bcl-xL inhibitors, that can be included as drug moieties in the PROTAC compounds described herein.

As used herein, a “Bcl-xL inhibitor drug moiety”, “Bcl-xL inhibitor moiety”, and the like refer to the component of the PROTAC compounds described herein that provides the structure of a Bcl-xL inhibitor compound or a compound modified for attachment to a DSM that retains essentially the same, similar, or enhanced biological function or activity as compared to the original compound. In some embodiments, Bcl-xL inhibitor drug moiety is component (D) in a compound of Formula (A).

The term “cancer,” as used herein, refers to the presence of cells possessing characteristics typical of cancer-causing cells, such as uncontrolled proliferation, immortality, metastatic potential, rapid growth and proliferation rate, and/or certain morphological features. Often, cancer cells can be in the form of a tumor or mass, but such cells may exist alone within a subject, or may circulate in the blood stream as independent cells, such as leukemic or lymphoma cells. The term “cancer” includes all types of cancers and cancer metastases, including hematological cancers, solid tumors, sarcomas, carcinomas and other solid and non-solid tumor cancers. Hematological cancers may include B-cell malignancies, cancers of the blood (leukemias), cancers of plasma cells (myelomas, e.g., multiple myeloma), or cancers of the lymph nodes (lymphomas). Exemplary B-cell malignancies include chronic lymphocytic leukemia (CLL), follicular lymphoma, mantle cell lymphoma, and diffuse large B-cell lymphoma. Leukemias may include acute lymphoblastic leukemia (ALL), acute myeloid leukemia (AML), chronic lymphocytic leukemia (CLL), chronic myelogenous leukemia (CML), chronic myelomonocytic leukemia (CMML), acute monocytic leukemia (AMoL), etc. Lymphomas may include Hodgkin's lymphoma, non-Hodgkin's lymphoma, etc. Other hematologic cancers may include myelodysplasia syndrome (MDS). Solid tumors may include carcinomas such as adenocarcinoma, e.g., breast cancer, pancreatic cancer, prostate cancer, colon or colorectal cancer, lung cancer, gastric cancer, cervical cancer, endometrial cancer, ovarian cancer, cholangiocarcinoma, glioma, melanoma, etc. In some embodiments, the cancer is a breast cancer, multiple myeloma, plasma cell myeloma, leukemia, lymphoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, chronic lymphocytic leukemia, prostate cancer, small cell lung cancer, or spleen cancer. In some embodiments, the cancer is a lymphoma or gastric cancer.

As used herein, the term “tumor” refers to any mass of tissue that results from excessive cell growth or proliferation, either benign or malignant, including precancerous lesions. In some embodiments, the tumor is a breast cancer, gastric cancer, bladder cancer, brain cancer, cervical cancer, colorectal cancer, esophageal cancer, hepatocellular cancer, melanoma, oral cancer, ovarian cancer, non-small cell lung cancer, prostate cancer, small cell lung cancer, or spleen cancer. In some embodiments, the tumor is a gastric cancer.

The terms “tumor cell” and “cancer cell” may be used interchangeably herein and refer to individual cells or the total population of cells derived from a tumor or cancer, including both non-tumorigenic cells and cancer stem cells. The terms “tumor cell” and “cancer cell” will be modified by the term “non-tumorigenic” when referring solely to those cells lacking the capacity to renew and differentiate to distinguish those cells from cancer stem cells.

The terms “subject” and “patient” are used interchangeably herein to refer to any human or non-human animal in need of treatment. Non-human animals include all vertebrates (e.g., mammals and non-mammals) such as any mammal. Non-limiting examples of mammals include humans, chimpanzees, apes, monkeys, cattle, horses, sheep, goats, swine, rabbits, dogs, cats, rats, mice, and guinea pigs. Non-limiting examples of non-mammals include birds and fish. In some embodiments, the subject is a human.

The term “a subject in need of treatment,” as used herein, refers to a subject that would benefit biologically, medically, or in quality of life from a treatment (e.g., a treatment with any one or more of the exemplary compounds described herein).

As used herein, the term “treat,” “treating,” or “treatment” refers to any improvement of any consequence of disease, disorder, or condition, such as prolonged survival, less morbidity, and/or a lessening of side effects which result from an alternative therapeutic modality. In some embodiments, treatment comprises delaying or ameliorating a disease, disorder, or condition (i.e., slowing or arresting or reducing the development of a disease or at least one of the clinical symptoms thereof). In some embodiments, treatment comprises delaying, alleviating, or ameliorating at least one physical parameter of a disease, disorder, or condition, including those which may not be discernible by the patient. In some embodiments, treatment comprises modulating a disease, disorder, or condition, either physically (e.g., stabilization of a discernible symptom), physiologically (e.g., stabilization of a physical parameter), or both. In some embodiments, treatment comprises administration of a described compound or composition to a subject, e.g., a patient, to obtain a treatment benefit enumerated herein. The treatment can be to cure, heal, alleviate, delay, prevent, relieve, alter, remedy, ameliorate, palliate, improve, or affect a disease, disorder, or condition (e.g., a cancer), the symptoms of a disease, disorder, or condition (e.g., a cancer), or a predisposition toward a disease, disorder, or condition (e.g., a cancer). In some embodiments, in addition to treating a subject having a disease, disorder, or condition, a composition disclosed herein can also be provided prophylactically to prevent or reduce the likelihood of developing that disease, disorder, or condition.

As used herein, the term “prevent”, “preventing,” or “prevention” of a disease, disorder, or condition refers to the prophylactic treatment of the disease, disorder, or condition; or delaying the onset or progression of the disease, disorder, or condition.

As used herein, a “pharmaceutical composition” refers to a preparation of a composition, e.g., an compound or composition, in addition to at least one other (and optionally more than one other) component suitable for administration to a subject, such as a pharmaceutically acceptable carrier, stabilizer, diluent, dispersing agent, suspending agent, thickening agent, and/or excipient. The pharmaceutical compositions provided herein are in such form as to permit administration and subsequently provide the intended biological activity of the active ingredient(s) and/or to achieve a therapeutic effect. The pharmaceutical compositions provided herein preferably contain no additional components which are unacceptably toxic to a subject to which the formulation would be administered.

As used herein, the terms “pharmaceutically acceptable carrier” and “physiologically acceptable carrier,” which may be used interchangeably, refer to a carrier or a diluent that does not cause significant irritation to a subject and does not abrogate the biological activity and properties of the administered compound or composition and/or any additional therapeutic agent in the composition. Pharmaceutically acceptable carriers may enhance or stabilize the composition or can be used to facilitate preparation of the composition. Pharmaceutically acceptable carriers can include solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. The carrier may be selected to minimize adverse side effects in the subject, and/or to minimize degradation of the active ingredient(s). An adjuvant may also be included in any of these formulations.

As used herein, the term “excipient” refers to an inert substance added to a pharmaceutical composition to further facilitate administration of an active ingredient. Formulations for parenteral administration can, for example, contain excipients such as sterile water or saline, polyalkylene glycols such as polyethylene glycol, vegetable oils, or hydrogenated napthalenes. Other exemplary excipients include, but are not limited to, calcium bicarbonate, calcium phosphate, various sugars and types of starch, cellulose derivatives, gelatin, ethylene-vinyl acetate co-polymer particles, and surfactants, including, for example, polysorbate 20.

The term “pharmaceutically acceptable salt,” as used herein, refers to a salt which does not abrogate the biological activity and properties of the compounds of the present disclosure, and does not cause significant irritation to a subject to which it is administered. Examples of such salts include, but are not limited to: (a) acid addition salts formed with inorganic acids, for example, hydrochloric acid, hydrobromic acid, sulfuric acid, phosphoric acid, nitric acid and the like; and salts formed with organic acids, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine. See, e.g., Haynes et al., “Commentary: Occurrence of Pharmaceutically Acceptable Anions and Cations in the Cambridge Structural Database,” J. Pharmaceutical Sciences, vol. 94, no. 10 (2005), and Berge et al., “Pharmaceutical Salts,” J. Pharmaceutical Sciences, vol. 66, no. 1 (1977), which are incorporated by reference herein.

In some embodiments, depending on their electronic charge, the PROTAC compounds, linkers, Bcl-xL inhibitors and linker-Bcl-xL inhibitors described herein can contain a monovalent anionic counterion M₁⁻. Any suitable anionic counterion can be used. In certain embodiments, the monovalent anionic counterion is a pharmaceutically acceptable monovalent anionic counterion. In certain embodiments, the monovalent anionic counterion M₁⁻ can be selected from bromide, chloride, iodide, acetate, trifluoroacetate, benzoate, mesylate, tosylate, triflate, formate, or the like. In some embodiments, the monovalent anionic counterion M₁⁻ is trifluoroacetate or formate.

As used herein, the term “therapeutically effective amount” or “therapeutically effective dose,” refers to an amount of a compound described herein, e.g., a PROTAC compound or composition described herein, to effect the desired therapeutic result (i.e., reduction or inhibition of an enzyme or a protein activity, amelioration of symptoms, alleviation of symptoms or conditions, delay of disease progression, a reduction in tumor size, inhibition of tumor growth, prevention of metastasis).

In some embodiments, a therapeutically effective amount does not induce or cause undesirable side effects. In some embodiments, a therapeutically effective amount induces or causes side effects but only those that are acceptable by a treating clinician in view of a patient's condition. In some embodiments, a therapeutically effective amount is effective for detectable killing, reduction, and/or inhibition of the growth or spread of cancer cells, the size or number of tumors, and/or other measure of the level, stage, progression and/or severity of a cancer. The term also applies to a dose that will induce a particular response in target cells, e.g., a reduction, slowing, or inhibition of cell growth.

A therapeutically effective amount can be determined by first administering a low dose, and then incrementally increasing that dose until the desired effect is achieved. A therapeutically effective amount can also vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific amount may vary depending on, for example, the particular pharmaceutical composition, the subject and their age and existing health conditions or risk for health conditions, the dosing regimen to be followed, the severity of the disease, whether it is administered in combination with other agents, timing of administration, the tissue to which it is administered, and the physical delivery system in which it is carried. In the case of cancer, a therapeutically effective amount of a PROTAC compound may reduce the number of cancer cells, reduce tumor size, inhibit (e.g., slow or stop) tumor metastasis, inhibit (e.g., slow or stop) tumor growth, and/or relieve one or more symptoms.

As used herein, the term “prophylactically effective amount” or “prophylactically effective dose,” refers to an amount of a compound disclosed herein, e.g., a PROTAC compound or composition described herein, that is effective, at dosages and for periods of time necessary, to achieve the desired prophylactic result. Typically, since a prophylactic dose is used in subjects prior to or at an earlier stage of disease, the prophylactically effective amount will be less than the therapeutically effective amount. In some embodiments, a prophylactically effective amount can prevent the onset of disease symptoms, including symptoms associated with a cancer.

### Protac Compounds

The PROTAC compounds of the present disclosure include those with anti-cancer activity. In particular, the PROTAC compounds include a degradation signaling moiety (DSM) conjugated (i.e., covalently attached by a linker) to a drug moiety (e.g., a Bcl-xL inhibitor), wherein the drug moiety when not conjugated to a DSM has a cytotoxic or cytostatic effect. In some embodiments, the drug moiety when not conjugated to a DSM is capable of reducing the expression and/or activity of Bcl-xL and/or one or more upstream modulators or downstream targets thereof. Without being bound by theory, by targeting Bcl-xL expression and/or activity, in some embodiments, the PROTAC compounds disclosed herein may provide potent anti-cancer agents. Also, without being bound by theory, by conjugating the drug moiety to a DSM that binds to an E3 ubiquitin ligase, the PROTAC compound may provide improved activity, better cytotoxic specificity, and/or reduced off-target killing as compared to the drug moiety when administered alone.

In some embodiments, therefore, the components of the PROTAC compounds are selected to (i) retain one or more therapeutic properties exhibited by the DSM in isolation, (ii) maintain the specific binding properties of the DSM; (iii) allow delivery, e.g., intracellular delivery, of the drug moiety via stable attachment to the DSM; (iv) retain PROTAC compound stability as an intact compound until transport or delivery to a target site; (v) allow for the therapeutic effect, e.g., cytotoxic effect, of the drug moiety after cleavage or other release mechanism in the cellular environment; (vi) exhibit in vivo anti-cancer treatment efficacy comparable to or superior to that of the DSM and drug moieties in isolation; (vii) minimize off-target killing by the drug moiety; and/or (viii) exhibit desirable pharmacokinetic and pharmacodynamics properties, formulatability, and toxicologic/immunologic profiles. Each of these properties may provide for a PROTAC compound for therapeutic use (Ab et al. (2015) Mol Cancer Ther. 14:1605-13).

Provided herein, in certain aspects, are PROTAC compounds comprising a degradation signaling moiety (DSM), a Bcl-xL inhibitor drug moiety (D), and a linker moiety (L) that covalently attaches DSM to D.

In some embodiments, PROTAC compounds of the present disclosure have the Formula (A):

D-L-DSM (A),

or an enantiomer, a diastereoisomer, and/or a pharmaceutically acceptable salt of any one of the forgoing, wherein DSM is a degradation signaling compound covalently attached to the linker L, L is a linker that covalently attaches DSM to D, and D is a Bcl-xL inhibitor compound that is covalently linked to the linker.

1. Bcl-xL Inhibitors

In some embodiments, the Bcl-xL inhibitor compound (D) of Formula (A) is represented by Formula (I) or Formula (II):

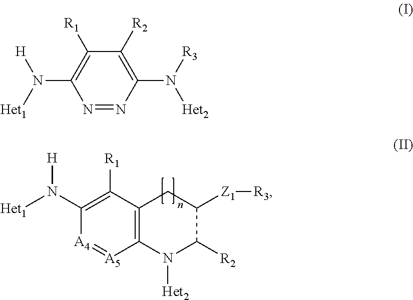

or an enantiomer, a diastereoisomer, and/or a pharmaceutically acceptable salt of any one of the foregoing, wherein the definitions of variables depicted in Formula (I) and (II) are described above (e.g., in the first or second embodiment).

In some embodiments, the Bcl-xL inhibitor compound (D) is represented by Formula (I), or an enantiomer, a diastereoisomer, and/or a pharmaceutically acceptable salt of any one of the foregoing, wherein R₁ is linear or branched C₁-C₆alkyl; R₂ is H; and the remaining variables are as described above for Formula (I).

In some embodiments, the Bcl-xL inhibitor compound (D) is represented by Formula (II), or an enantiomer, a diastereoisomer, and/or a pharmaceutically acceptable salt of any one of the foregoing, wherein A₄ and A₅ both represent a nitrogen atom, R₁ is linear or branched C₁₋₆alkyl, R₂ is H, n is 1, ----- in Formula (II) represents a single bond, and the remaining variables are as described above for Formula (II).

In some embodiments, the Bcl-xL inhibitor compound (D) is represented by Formula (IA) or (IIA):

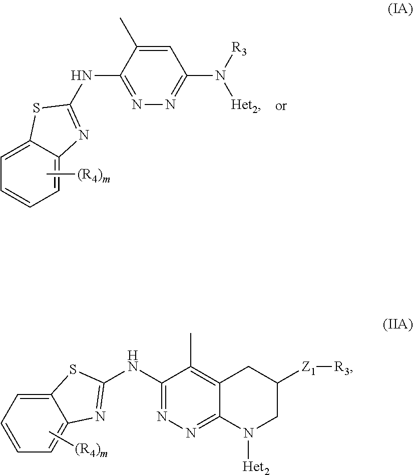

or an enantiomer, a diastereoisomer, and/or a pharmaceutically acceptable salt of any one of the foregoing, wherein:
Z₁ represents a bond or —O—,
R₃ represents a group selected from the group consisting of: hydrogen; a C₃-C₆cycloalkyl; a linear or branched C₁-C₆alkyl; —X₁—NRₐR_{b}; —X₁—N⁺RₐR_{b}R_{c}; —X₁—O—R_{c}; —X₁—N₃ and —X₁-≡CH,
Rₐ and R_{b} independently of one another represent a group selected from the group consisting of: hydrogen; a linear or branched C₁-C₆alkyl optionally substituted by one or two hydroxyl groups; and a C₁-C₆alkylene-SO₂O⁻,
R_{c} represents a hydrogen or a linear or branched C₁-C₆alkyl group,
Het₂ represents a group selected from the group consisting of:

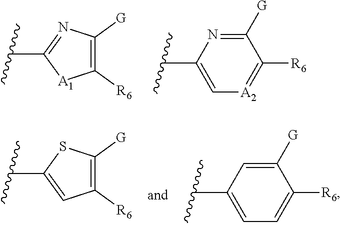

A₁ is —NH—, —N(C₁-C₃alkyl), O, S or Se,
A₂ is N, CH or C(R₅),
G is selected from the group consisting of:
   —C(O)OH, —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, —C₁-C₆alkyl optionally substituted by a hydroxyl group, —C(O)NR_{G5}S(O)₂R_{G4}, halogen, —NO₂, and —CN, in which:
R_{G1}R_{G2}, R_{G4} and R_{G5} at each occurrence are each independently selected from the group consisting of hydrogen, and a C₁-C₆alkyl optionally substituted by 1 to 3 halogen atoms;
R_{G3} is a C₁-C₆alkyl optionally substituted by 1 to 3 halogen atoms; or
   R_{G1} and R_{G2}, together with the atom to which each is attached are combined to form a C₃-C₈heterocycloalkyl;
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a halogen and —CN,
Rₑ represents a group selected from the group consisting of:
a linear or branched —C₁-C₆alkylene-R₈ group;
—X₂—O—R₇; and
a heteroarylene-R₇ group optionally substituted by a linear or branched C₁-C₆alkyl group,
R₇ represents a group selected from the group consisting of: a linear or branched C₁-C₆alkyl group; (C₃-C₆)cycloalkylene-R₈;

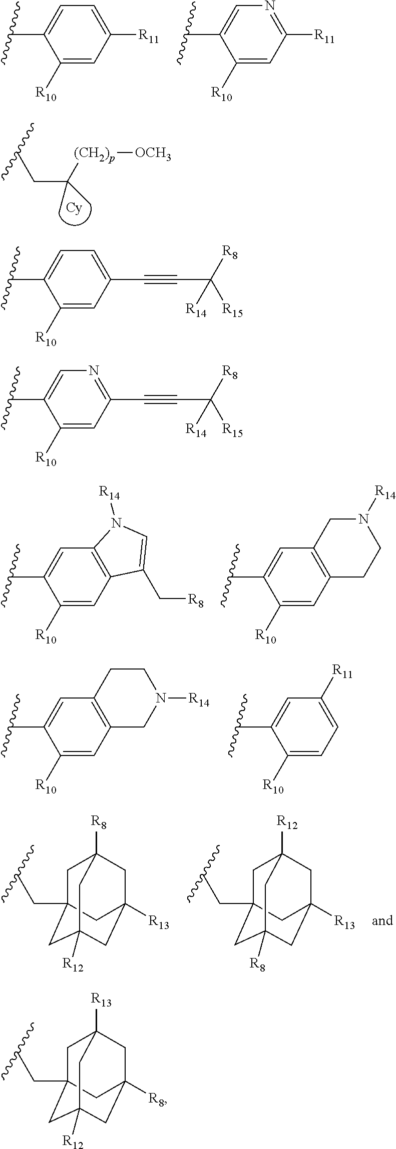

wherein Cy represents a C₃-C₈cycloalkyl,
R₈ represents a group selected from the group consisting of: hydrogen; a linear or branched C₁-C₆alkyl, —NR′ₐR′_{b}; —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}; —X′₂—NR′ₐR′_{b}; —NR′_{c}—X′₂—N₃ and:
   —NR′_{c}—X′₂-≡CH,
R₁₀ represents a group selected from the group consisting of hydrogen, fluorine, chlorine, bromine, —CF₃ and methyl,
R₁₁ represents a group selected from the group consisting of hydrogen, a C₁-C₃alkylene-R₈, —O—C₁-C₃alkylene-R₈, —CO—NRₕRᵢ, —CH═CH—C₁-C₄alkylene-NRₕRᵢ, —CH═CH—CHO, a C₃-C₈cycloalkylene-CH₂—R₈, and a C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a
a cyclohexyl,
Rₕ and Rᵢ, independently of one another, represent a hydrogen or a linear or branched C₁-C₆alkyl group,
X₁ and X₂ independently of one another, represent a linear or branched C₁-C₆alkylene group optionally substituted by one or two groups selected from the group consisting of trifluoromethyl, hydroxyl, a halogen, and C₁-C₆alkoxy,
X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O—; a C₁-C₆alkylene-COOH; a C₁-C₆alkylene-PO(OH)₂; a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy group; and the group:

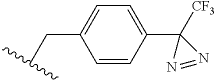 [SMILES: *Cc1ccc(C2(C(F)(F)F)N=N2)cc1]

or R′ₐ and R′_{b} form with the nitrogen atom carrying them a cycle B₃,
or R′ₐ, R′_{b} and R′_{c} form with the nitrogen atom carrying them a bridged C₃-C₈heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₆heterocycloalkyl,
m=0, 1 or 2,
p=1, 2, 3 or 4,
   B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl.

In some embodiments, the Bcl-xL inhibitor compound (D) is represented by Formula (IA) or (IIA):

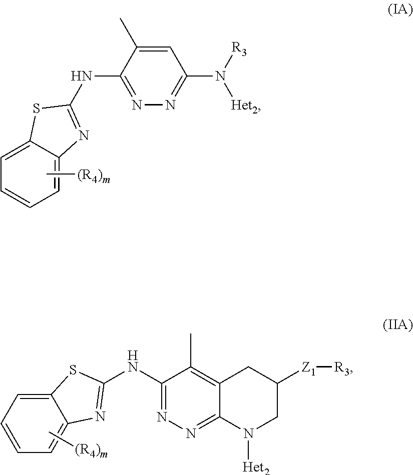

or an enantiomer, a diastereoisomer, and/or a pharmaceutically acceptable salt of any one of the foregoing, wherein:
Z₁ represents a bond or —O—,
R₃ represents a group selected from: hydrogen; C₃-C₆cycloalkyl; linear or branched C₁-C₆alkyl; —X₁—NRₐR_{b}; —X₁—N⁺RₐR_{b}R_{c}; —X₁—O—R_{c}; —X₁—N₃ and —X₁-≡CH,
Rₐ and R_{b} independently of one another represent a group selected from: hydrogen; linear or branched C₁-C₆alkyl optionally substituted by one or two hydroxyl groups; and
C₁-C₆alkylene-SO₂O—,
R_{c} represents a hydrogen or a linear or branched C₁-C₆alkyl group,
Het₂ represents a group selected from:

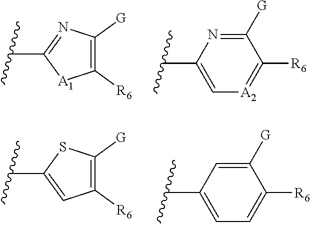

A₁ is —NH—, —N(C₁-C₃alkyl), O, S or Se,
A₂ is N, CH or C(R₅),
G is selected from the group consisting of:
—C(O)OH, —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, halogen, —NO₂, and —CN, in which:
R_{G1} and R_{G2} at each occurrence are each independently selected from the group consisting of hydrogen, and C₁-C₆alkyl optionally substituted by 1 to 3 halogen atoms;
R_{G3} is C₁-C₆alkyl optionally substituted by 1 to 3 halogen atoms; or R_{G1} and R_{G2}, together with the atom to which each is attached are combined to form a C₃-C₆heterocycloalkyl;
R₄ represents a hydrogen, fluorine, chlorine or bromine atom, a methyl, a hydroxyl or a methoxy group,
R₅ represents a group selected from: C₁-C₆alkyl optionally substituted by 1 to 3 halogen atoms; halogen or —CN,
R₆ represents a group selected from:
a linear or branched —C₁-C₆alkylene-Rₐ group;
—X₂—O—R₇; and
a heteroarylene-R₇ group optionally substituted by a linear or branched C₁-C₆alkyl group,
R₇ represents a group selected from: linear or branched C₁-C₆alkyl group; (C₃-C₆)cycloalkylene-R₈; or:

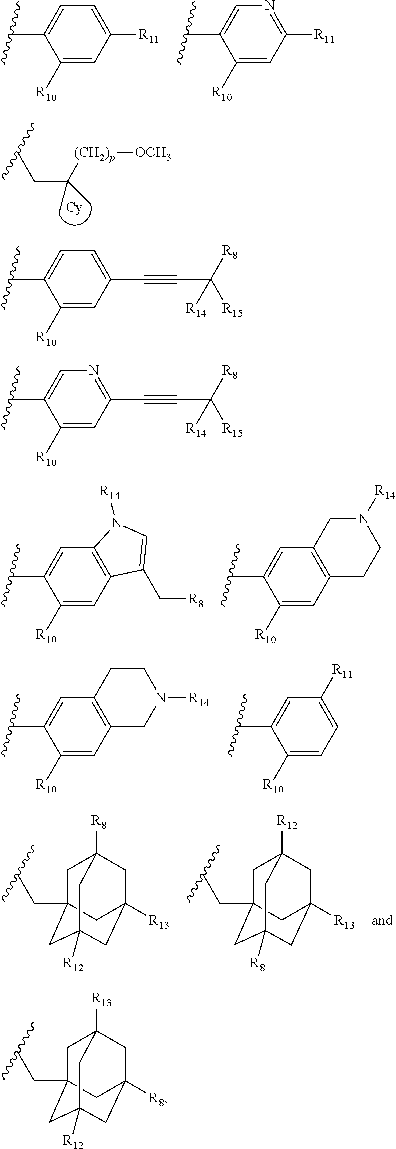

wherein Cy represents a C₃-C₈cycloalkyl,
R₈ represents a group selected from: hydrogen; linear or branched C₁-C₆alkyl, —NR′ₐR′_{b};
   —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}; —X′₂—NR′ₐR′_{b}: —NR′_{c}—X′₂—N₃ and —NR′_{c}—X′₂-≡CH,
R₁₀ represents a group selected from hydrogen, fluorine, chlorine, bromine, —CF₃ and methyl,
R₁₁ represents a group selected from hydrogen, C₁-C₃alkylene-R₈, —O—C₁-C₃alkylene-R₈, —CO—NRₕRᵢ and —CH═CH—C₁-C₄alkylene-NRₕRᵢ, —CH═CH—CHO, C₃-C₈cycloalkylene-CH₂—R₈, C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a a cyclohexyl,
Rₕ and Rᵢ, independently of one another, represent a hydrogen or a linear or branched C₁-C₆alkyl group,
X₁ and X₂ independently of one another, represent a linear or branched C₁-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl or C₁-C₆alkoxy groups; C₁-C₆alkylene-SO₂OH; C₁-C₆alkylene-SO₂O—; C₁-C₆alkylene-COOH; C₁-C₆alkylene-PO(OH)₂; C₁-C₆alkylene-NR′_{d}R′ₑ; C₁-C₆alkylene-N⁺R′_{d}R′ₑR′_{f}; C₁-C₆alkylene-O—C₁-C₆alkylene-OH; C₁-C₆alkylene-phenyl wherein the phenyl may be substituted by a hydroxyl or a C₁-C₆alkoxy group;
the group:

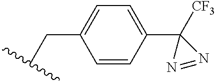 [SMILES: *Cc1ccc(C2(C(F)(F)F)N=N2)cc1]

or R′ₐ and R′_{b} form with the nitrogen atom carrying them a cycle B₃,
or R′ₐ, R′_{b} and R′_{c} form with the nitrogen atom carrying them a bridged C₃-C₆heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈heterocycloalkyl,
m=0, 1 or 2,
   B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from: fluorine, bromine, chlorine, linear or branched C₁-C₆alkyl, hydroxyl, —NH₂, oxo or piperidinyl.

In some embodiments, for Formula (I), (II), (IA), or (IIA), R₇ represents a group selected from: linear or branched C₁-C₆ alkyl group; (C₃-C₆)cycloalkylene-R₈; or:

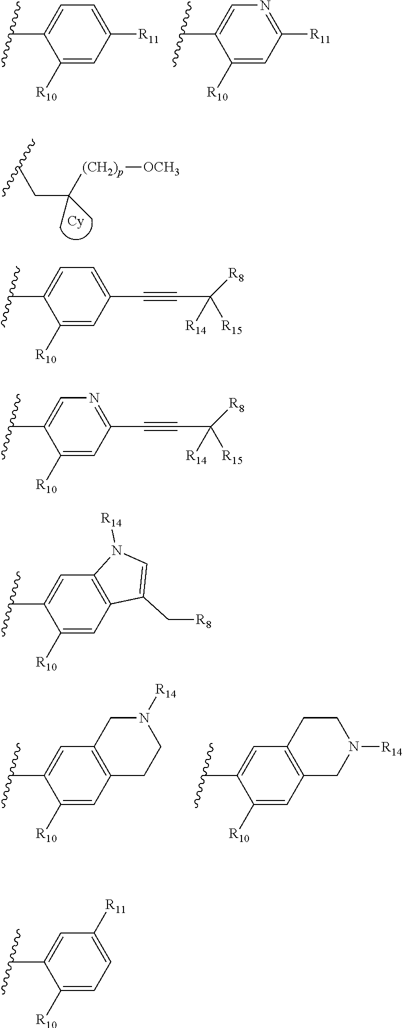

wherein Cy represents a C₃-C₈cycloalkyl.

In some embodiments, for Formula (I), (II), (IA), or (IIA), R₇ represents a group selected from:

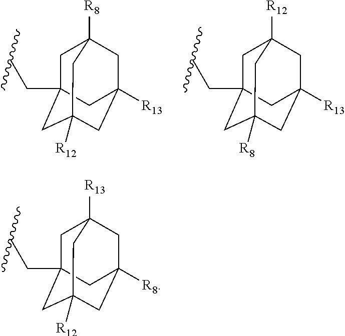

In some embodiments, the Bcl-xL inhibitor compound (D) is represented by Formula (IB), (IC-1), (IIB) or (IIC-1):

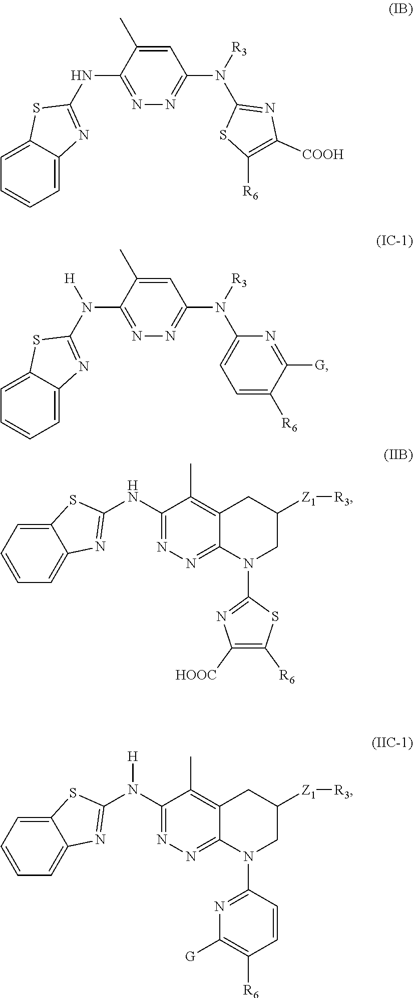

or an enantiomer, a diastereoisomer, and/or a pharmaceutically acceptable salt of any one of the foregoing, wherein:
for formula (IB) or (IC-1), R₃ represents a group selected from the group consisting of: hydrogen; a linear or branched C₁-C₆alkyl; —X₁—NRₐR_{b}; —X₁—N⁺RₐR_{b}R_{c}; —X₁—O—R_{c}; —X₁—N₃ and —X₁-≡CH,
for formula (IIB) or (IIC-1), Z₁ represents a bond, and R₃ represents hydrogen; or Z₁ represents —O—, and R₃ represents —X₁—NRₐR_{b},
for formula (IC-1), G is selected from the group consisting of —C(O)OH and —C(O)NR_{G1}R_{G2};
for formula (IIC-1), G is selected from the group consisting of —C(O)OH, —C(O)NR_{G1}R_{G2}, —C(O)R_{G2}, —C₁-C₆alkyl optionally substituted by a hydroxyl group and —C(O)NR_{G5}S(O)₂R_{G4}, in which R_{G1}, R_{G2}, R_{G4} and R_{G5} at each occurrence are each independently selected from the group consisting of hydrogen and a C₁-C₆alkyl optionally substituted by 1 to 3 halogen atoms;
Rₐ and R_{b} independently of one another represent a group selected from the group consisting of: hydrogen; a linear or branched C₁-C₆alkyl optionally substituted by one or two hydroxyl groups; and a C₁-C₆alkylene-SO₂O—,
R_{c} represents a hydrogen or a linear or branched C₁-C₆alkyl group
R₆ represents a linear or branched —C₁-C₆alkylene-Rₐ group, —X₂—O—R₇, or a heteroarylene-R₇ group optionally substituted by a linear or branched C₁-C₆alkyl group,
R₇ represents a group selected from the group consisting of:

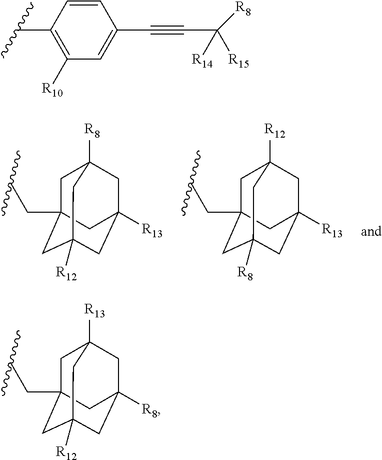

R₈ represents a group selected from the group consisting of: —NR′ₐR′_{b}; —O—X′₂—NR′ₐR′_{b}; and —X′₂—NR′ₐR′_{b},
R₁₀ represents fluorine,
R₁₂ and R₁₃, independently of one another, represent a hydrogen atom or a methyl group,
R₁₄ and R₁₅, independently of one another, represent a hydrogen or a methyl group,
X₁ and X₂ independently of one another, represent a linear or branched C₁-C₆alkylene group optionally substituted by one or two groups selected from the group consisting of trifluoromethyl, hydroxyl, a halogen, and a C₁-C₆alkoxy,
X′₂ represents a linear or branched C₁-C₆alkylene,
R′ₐ and R′_{b} independently of one another, represent a group selected from the group consisting of: hydrogen; a linear or branched C₁-C₆alkyl optionally substituted by one or two hydroxyl or C₁-C₆alkoxy groups; and a C₁-C₆alkylene-NR′_{d}R′ₑ; or R′ₐ and R′_{b} form with the nitrogen atom carrying them a cycle B₃,
R′_{d}, R′ₑ independently of one another, represents a hydrogen or a linear or branched C₁-C₆alkyl group,
   B₃ represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen and nitrogen, (iii) be substituted by one or two groups selected from the group consisting of: fluorine, bromine, chlorine, a linear or branched C₁-C₆alkyl, hydroxyl, and oxo.

In some embodiments, the Bcl-xL inhibitor compound (D) is represented by Formula (IB), (IC) (IIB) or (IIC):

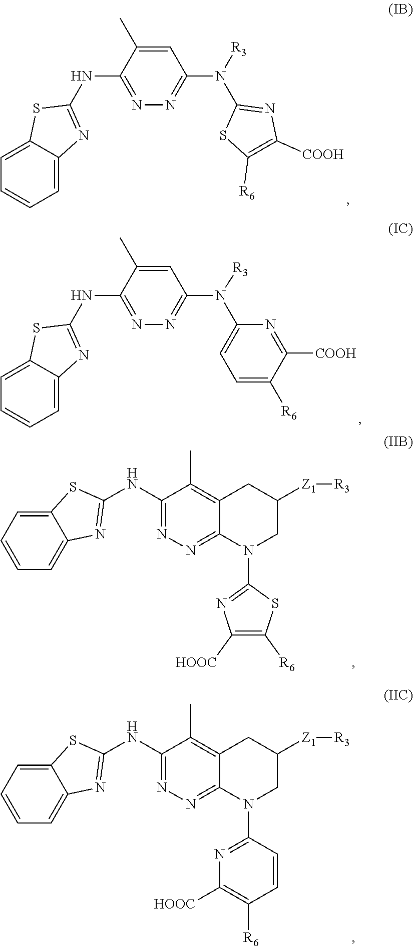

or an enantiomer, a diastereoisomer, and/or a pharmaceutically acceptable salt thereof, wherein:
for formula (IB) or (IC), R₃ represents a group selected from: hydrogen; linear or branched C₁-C₆alkyl; —X₁—NRₐR_{b}; —X₁—N⁺RₐR_{b}R_{c}; —X₁—O—R₁; —X₁—N₃ and —X₁-≡CH, for formula (IIB) or (IIC), Z₁ represents a bond, and R₃ represents hydrogen; or Z, represents —O—, and R₃ represents —X₁—NRₐR_{b},
Rₐ and R_{b} independently of one another represent a group selected from: hydrogen; linear or branched C₁-C₆alkyl optionally substituted by one or two hydroxyl groups; and
C₁-C₆alkylene-SO₂O—,
R_{c} represents a hydrogen or a linear or branched C₁-C₆alkyl group
R₆ represents a linear or branched —C₁-C₆alkylene-Rₐ group, —X₂—O—R₇, or a heteroarylene-R₇ group optionally substituted by a linear or branched C₁-C₆alkyl group,
R₇ represents a group selected from:

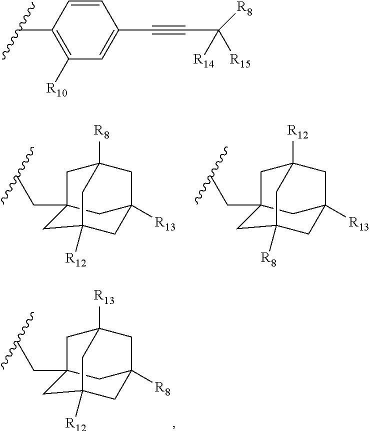

R₈ represents a group selected from: —NR′ₐR′_{b}; —O—X′₂—NR′ₐR′_{b}; and —X′₂—NR′ₐR′_{b},
R₁₀ represents fluorine,
R₁₂ and R₁₃, independently of one another, represent a hydrogen atom or a methyl group,
R₁₄ and R₁₅, independently of one another, represent a hydrogen or a methyl group,
X₁ and X₂ independently of one another, represent a linear or branched C₀-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; linear or branched C₁-C₆alkyl optionally substituted by one or two hydroxyl or C₁-C₆alkoxy groups; C₁-C₆alkylene-NR′_{d}R′ₑ;
or R′ₐ and R′_{b} form with the nitrogen atom carrying them a cycle B₃,
R′_{d}, R′ₑ independently of one another, represents a hydrogen or a linear or branched C₁-C₆alkyl group,
   B₃ represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen and nitrogen, (iii) be substituted by one or two groups selected from: fluorine, bromine, chlorine, linear or branched C₁-C₆alkyl, hydroxyl, and oxo.

In some embodiments, for Formula (I), (II), (IA), (IIA), (IB), (IIB), (IC), (IC-1), (IIC) or (IIC-1), Rₑ represents —X₂—O—R₇, and R₇ represents the following group:

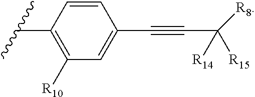

In some embodiments, for Formula (I), (II), (IA), (IIA), (IB), (IIB), (IC), (IC-1), (IIC) or (IIC-1), R₆ represents a heteroarylene-R₇ group optionally substituted by a linear or branched C₁-C₆alkyl group, and R₇ represents a group selected from:

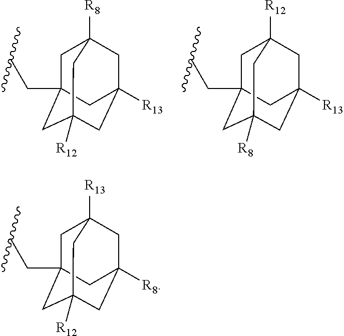

In some embodiments, for Formula (I), (II), (IA), (IIA), (IB), (IIB), (IC), (IC-1), (IIC) or (IIC-1), B₃ represents a C₃-C₆heterocycloalkyl group selected from a pyrrolidinyl group, a piperidinyl group, a piperazinyl group, a morpholinyl group, an azepanyl group, and a 4,4-difluoropiperidin-1-yl group.

In some embodiments, for Formula (I), (II), (IA), (IIA), (IB), (IIB), (IC), (IC-1), (IIC) or (IIC-1), B₃ represents a pyrrolidinyl group or a piperazinyl group.

In some embodiments, for Formula (I), (II), (IA), (IIA), (IB), (IIB), (IC), (IC-1), (IIC) or (IIC-1), B₃ represents a piperazinyl group.

In some embodiments, for Formula (I), (II), (IA), (IIA), (IB), (IIB), (IC), (IC-1), (IIC) or (IIC-1), Ra represents a group selected from the group consisting of:

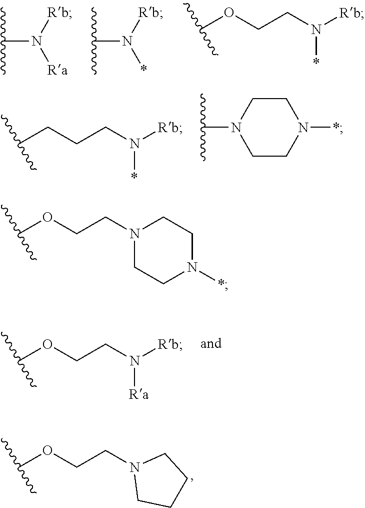

wherein:
-* is a bond to the linker.

In some embodiments, for Formula (I), (II), (IA), (IIA), (IB), (IIB), (IC), (IC-1), (IIC) or (IIC-1), R₈ represents a group selected from:

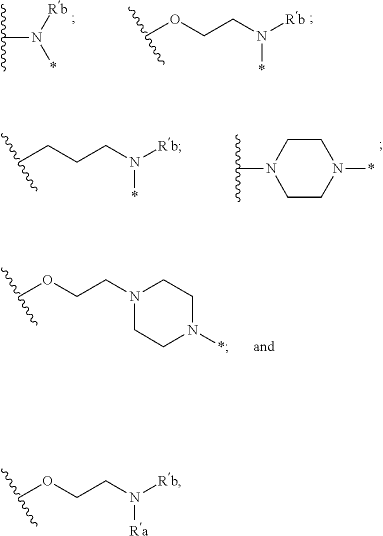

wherein -* is a bond to the linker.

In some embodiments, R₈ represents a group selected from:

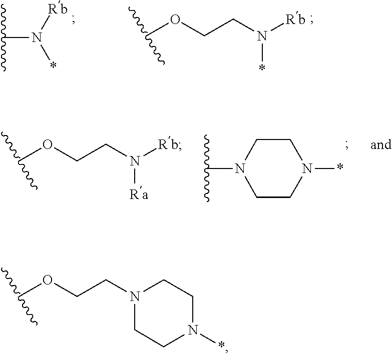

wherein -* is a bond to the linker.

In some embodiments, the Bcl-xL inhibitor compound (D) is represented by Formula (IB), (IIB), (IC), (IC-1), (IIC) or (IIC-1), or an enantiomer, a diastereoisomer, and/or a pharmaceutically acceptable salt of any one of the foregoing, wherein:
for formula (IB) or (IC-1), R₃ represents a group selected from the group consisting of: hydrogen; a linear or branched C₁-C₆alkyl; —X₁—NRₐR_{b}; —X₁—N₃ and —X₁-≡CH,
for formula (IIB) or (IIC-1), Z₁ represents a bond, and R₃ represents hydrogen,
for formula (IC-1), G is selected from the group consisting of —C(O)OH and —C(O)N(CH₃)₂;
for formula (IIC-1), G is selected from the group consisting of —C(O)NHS(O)₂H, —C(O)NH₂, —C(O)NHCH₃, —C(O)NHC(CH₃)₂, —C(O)N(CH₃)₂, —C(O)OH, and —CH₂OH;
   R₆ represents a linear or branched —C₁-C₆alkylene-Rₐ group, —X₂—O—R₇ or a heteroarylene-R₇ group optionally substituted by a linear or branched C₁-C₆alkyl group,
R₇ represents a group selected from the group consisting of:

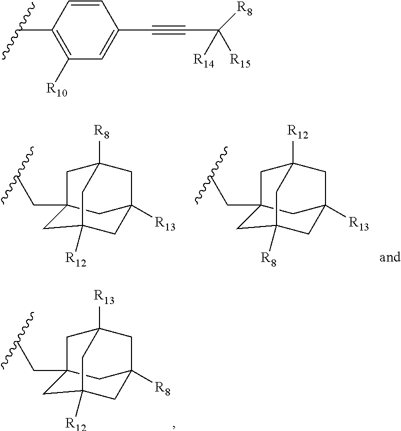

R₈ represents a group selected from the group consisting of: —NR′ₐR′_{b}; and —O—X′₂—NR′ₐR′_{b},
R₁₀ represents fluorine,
R₁₂ and R₁₃, independently of one another, represent a hydrogen atom or a methyl group,
R₁₄ and R₁₅, independently of one another, represent a hydrogen or a methyl group,
X₁ and X₂ independently of one another, represent a linear or branched C₁-C₆alkylene group optionally substituted by one or two groups selected from the group consisting of trifluoromethyl, hydroxyl, a halogen, and C₁-C₆alkoxy,
X′₂ represents a linear or branched C₁-C₆alkylene,
R′ₐ and R′_{b} independently of one another, represent a group selected from the group consisting of: hydrogen; a linear or branched C₁-C₆alkyl optionally substituted by one or two hydroxyl or C₁-C₆alkoxy groups; and a C₁-C₆alkylene-NR′_{d}R′ₑ; or R′ₐ and R′_{b} form with the nitrogen atom carrying them a cycle B₃,
R′_{d}, R′ₑ independently of one another, represents a hydrogen or a linear or branched C₁-C₆alkyl group,
B₃ represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen and nitrogen, (iii) be substituted by one or two groups selected from the group consisting of: fluorine, bromine, chlorine, linear or a branched C₁-C₆alkyl, hydroxyl, and oxo.

In some embodiments, the Bcl-xL inhibitor compound (D) is represented by Formula (IB), (IIB), (IC), (IC-1), (IIC) or (IIC-1), or an enantiomer, a diastereoisomer, and/or a pharmaceutically acceptable salt of any one of the foregoing, wherein:
for formula (IB) or (IC), R₃ represents a group selected from: hydrogen; linear or branched C₁-C₆alkyl; —X₁—N₃ and —X₁-≡CH,
for formula (IIB) or (IIC), Z₁ represents a bond, and R₃ represents hydrogen,
R₆ represents a linear or branched —C₁-C₆alkylene-Rₐ group, —X₂—O—R₇ or a heteroarylene-R₇ group optionally substituted by a linear or branched C₁-C₆alkyl group,
R₇ represents a group selected from:

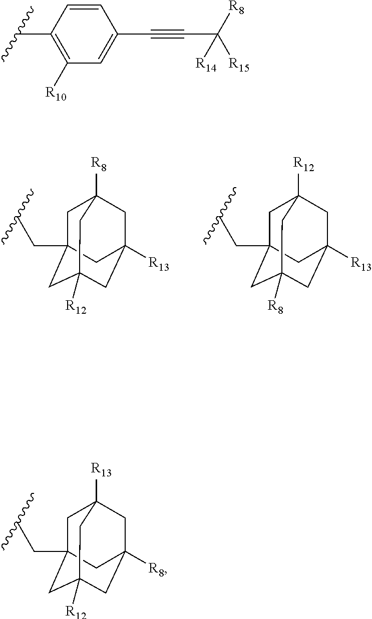

R₈ represents a group selected from: —NR′ₐR′_{b}; and —O—X′₂—NR′ₐR′_{b},
R₁₀ represents fluorine,
R₁₂ and R₁₃, independently of one another, represent a hydrogen atom or a methyl group,
R₁₄ and R₁₅, independently of one another, represent a hydrogen or a methyl group,
X₁ and X₂ independently of one another, represent a linear or branched C₁-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; linear or branched C₁-C₆alkyl optionally substituted by one or two hydroxyl or C₁-C₆alkoxy groups; C₁-C₆alkylene-NR′_{d}R′ₑ;
or R′ₐ and R′_{b} form with the nitrogen atom carrying them a cycle B₃,
R′_{d}, R′ₑ independently of one another, represents a hydrogen or a linear or branched C₁-C₆alkyl group,
B₃ represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen and nitrogen, (iii) be substituted by one or two groups selected from: fluorine, bromine, chlorine, linear or branched C₁-C₆alkyl, hydroxyl, and oxo.

In some embodiments, the Bcl-xL inhibitor compound (0) comprises a Bcl-xL inhibitor known in the art, for example, ABT-737 and ABT-263.

In some embodiments, 0 represents a Bcl-xL inhibitor attached to the linker L by a covalent bond, wherein the Bcl-xL inhibitor is selected from a compound in Table 1, or an enantiomer, a diastereomer and/or a pharmaceutically acceptable salt thereof.

TABLE 1
Exemplary Bcl-xL inhibitors

| | |
|---|---|
| D1 | 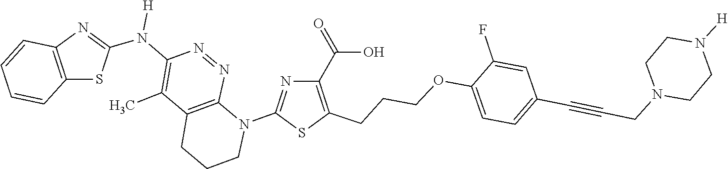 [SMILES: Cc1c(Nc2nc3ccccc3s2)nnc2c1CCCN2c1nc(C(=O)O)c(CCCOc2ccc(C#CCN3CCNCC3)cc2F)s1] |
| D2 | 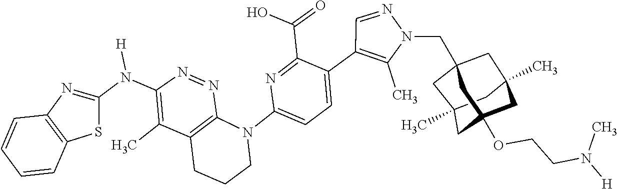 |
| D3 | 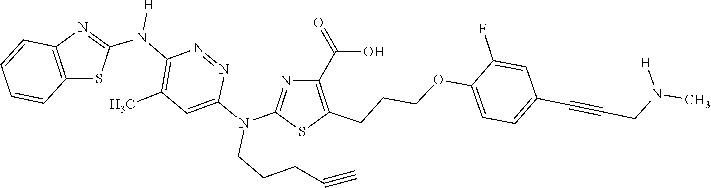 [SMILES: C#CCCCN(c1cc(C)c(Nc2nc3ccccc3s2)nn1)c1nc(C(=O)O)c(CCCOc2ccc(C#CCNC)cc2F)s1] |
| D4 | 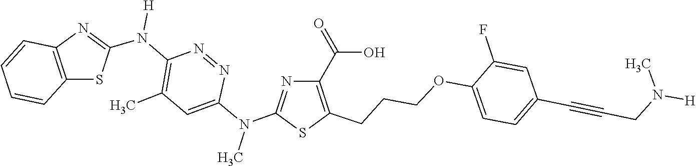 [SMILES: CNCC#Cc1ccc(OCCCc2sc(N(C)c3cc(C)c(Nc4nc5ccccc5s4)nn3)nc2C(=O)O)c(F)c1] |
| D5 | 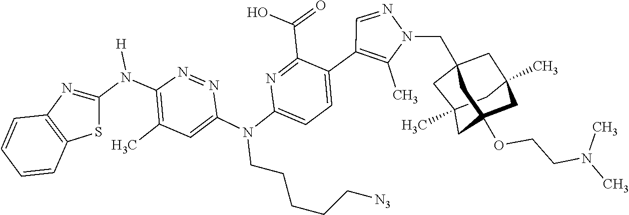 |
| D6 | 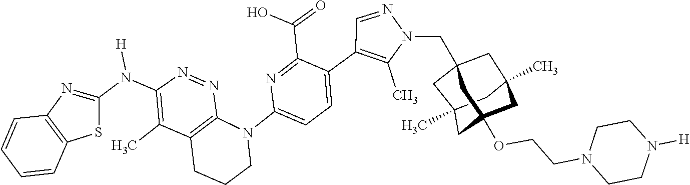 |
| D7 | 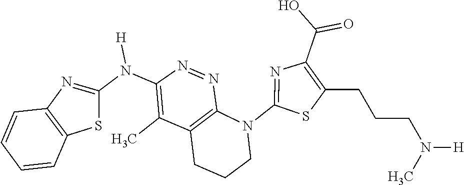 [SMILES: CNCCCc1sc(N2CCCc3c2nnc(Nc2nc4ccccc4s2)c3C)nc1C(=O)O] |
| D8 | 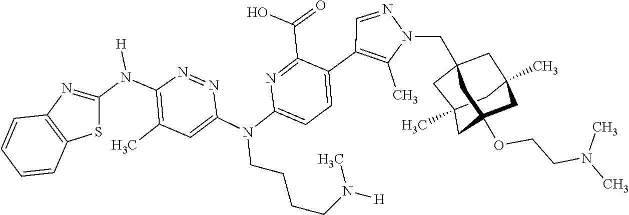 |
| D9 | 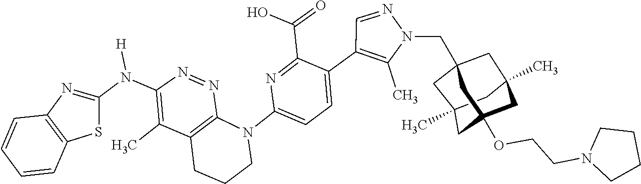 |
| D10 | 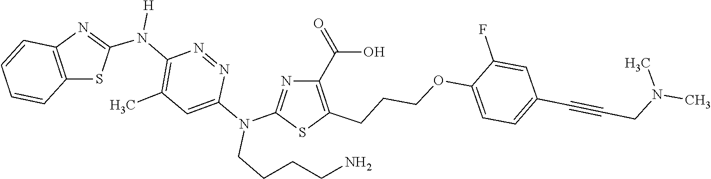 [SMILES: Cc1cc(N(CCCCN)c2nc(C(=O)O)c(CCCOc3ccc(C#CCN(C)C)cc3F)s2)nnc1Nc1nc2ccccc2s1] |
| D11 | 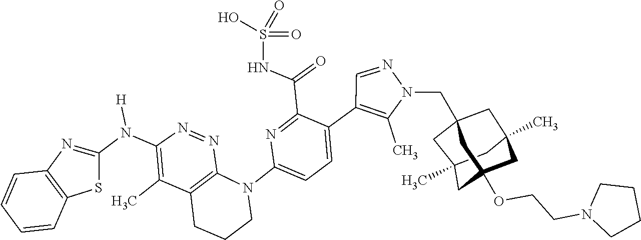 |
| D12 | 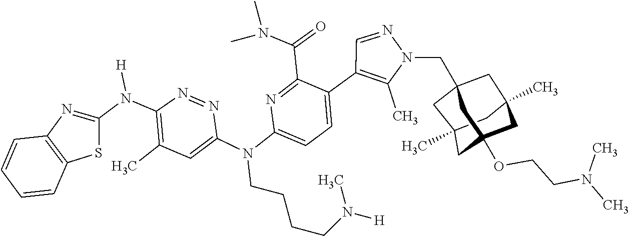 |
| D13 | 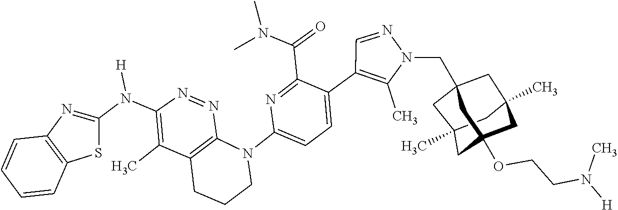 |
| D14 | 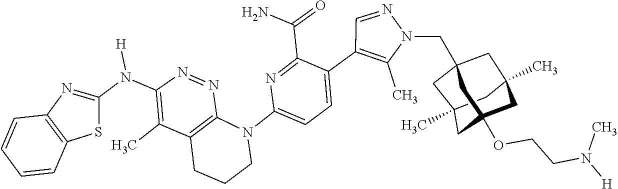 |
| D15 | 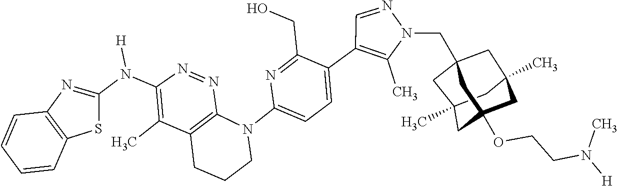 |
| D16 | 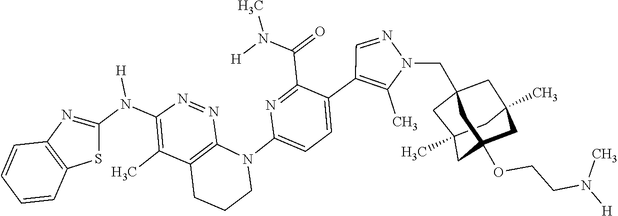 |
| D17 | 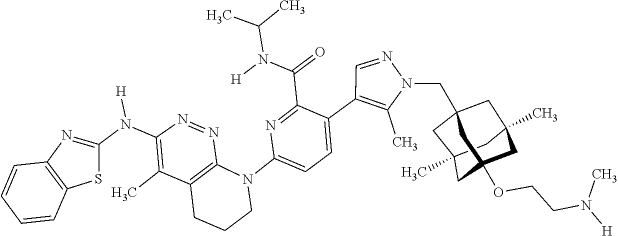 |

In some embodiments, D represents a moiety selected from any one of the formulae in Table 2, or an enantiomer, a diastereomer, and/or a pharmaceutically acceptable salt thereof, where -* represents a bond to the linker (L).

TABLE 2
Exemplary Bcl-xL Moieties Showing Point of Attachment to the Linker (L)

| | |
|---|---|
| D1a | 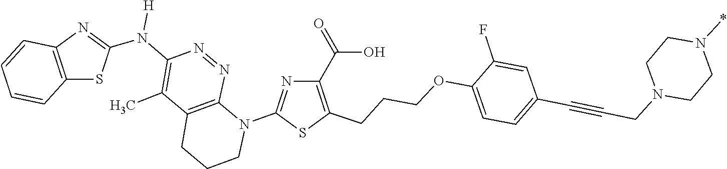 [SMILES: *N1CCN(CC#Cc2ccc(OCCCc3sc(N4CCCc5c4nnc(Nc4nc6ccccc6s4)c5C)nc3C(=O)O)c(F)c2)CC1] |
| D2a | 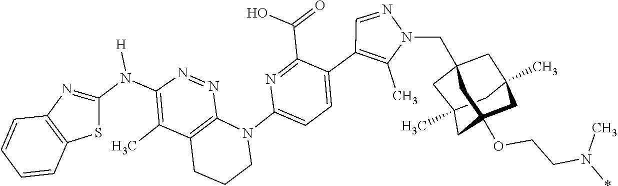 |
| D3a | 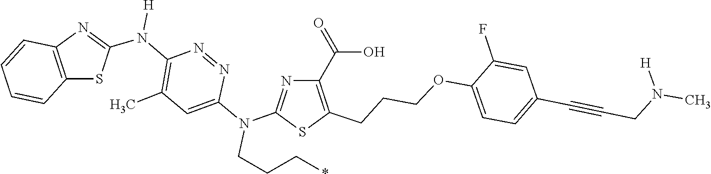 [SMILES: *CCCN(c1cc(C)c(Nc2nc3ccccc3s2)nn1)c1nc(C(=O)O)c(CCCOc2ccc(C#CCNC)cc2F)s1] |
| D4a | 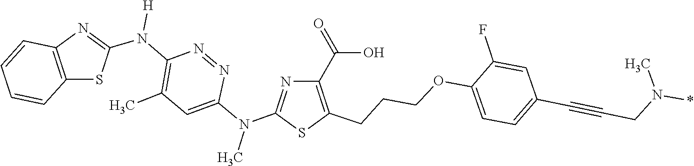 [SMILES: *N(C)CC#Cc1ccc(OCCCc2sc(N(C)c3cc(C)c(Nc4nc5ccccc5s4)nn3)nc2C(=O)O)c(F)c1] |
| D5a | 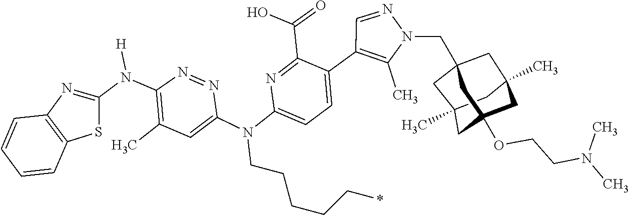 |
| D6a | 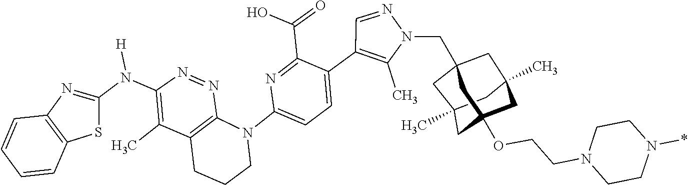 |
| D7a | 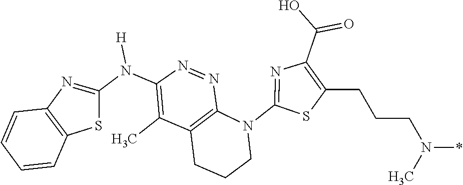 [SMILES: *N(C)CCCc1sc(N2CCCc3c2nnc(Nc2nc4ccccc4s2)c3C)nc1C(=O)O] |
| D8a | 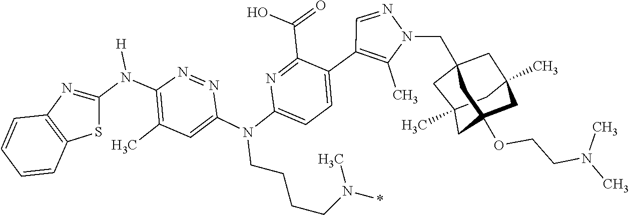 |
| D9a | 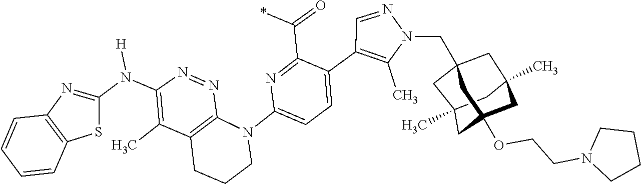 |
| D10a | 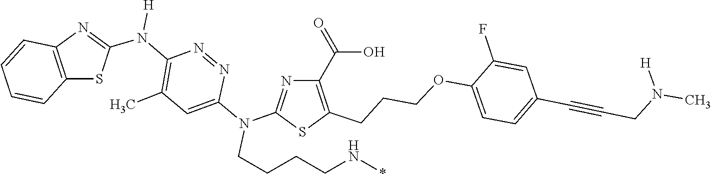 [SMILES: *NCCCCN(c1cc(C)c(Nc2nc3ccccc3s2)nn1)c1nc(C(=O)O)c(CCCOc2ccc(C#CCNC)cc2F)s1] |
| D11a | 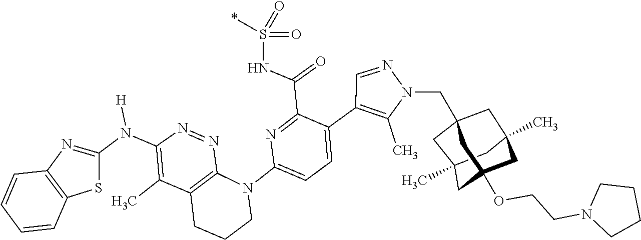 |
| D12a | 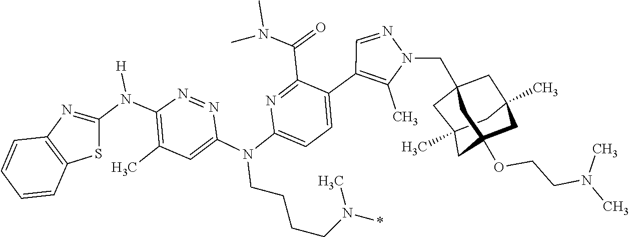 |
| D13a | 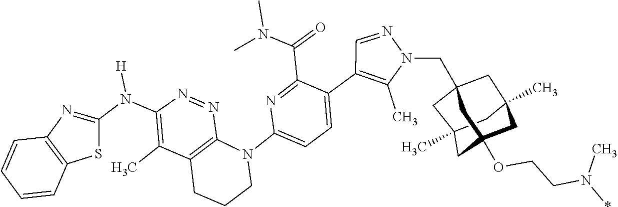 |
| D14a | 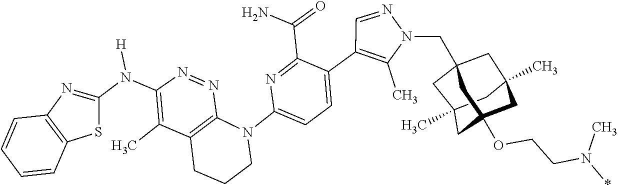 |
| D15a | 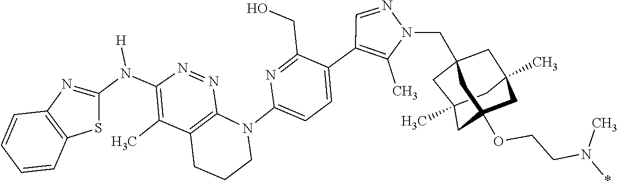 |
| D16a | 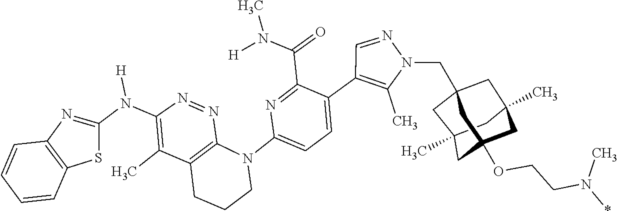 |
| D17a | 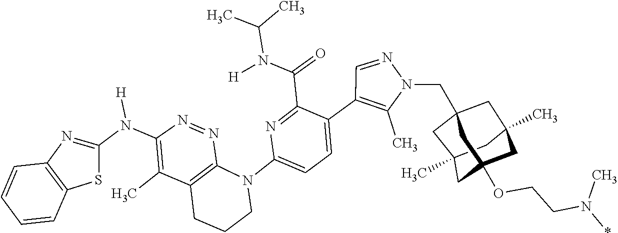 |

2. Linkers

In some embodiments, a bifunctional linker compound can be used to covalently attach a degradation signaling compound to a Bcl-xL inhibitor drug compound to form the PROTAC compounds of the present disclosure comprising a degradation signaling moiety (DSM) and a Bcl-xL inhibitor drug moiety (D). The bifunctional linker compound has at one end a reactive group that can react with the Bcl-xL inhibitor compound and at the other end another reactive group that can react with the degradation signaling compound. In some embodiment, The bifunctional linker compound is reacted with the drug moiety (e.g., the Bcl-xL inhibitor) under appropriate conditions. The product of the reaction, a drug-linker compound, is subsequently reacted with the degradation signaling compound, under conditions to form the PROTAC compound of the present disclosure. Alternatively, the linker compound can first react with the degradation signaling compound, to form a linker-DSM compound, which can then react with the drug to obtain the PROTAC compound of the present disclosure.

In some embodiments, when the Bcl-xL inhibitor compound is connected to a linker compound by means of a reactive azide or alkyne group, then a resulting triazole group is considered to be part of L. In some embodiments, when the Bcl-xL inhibitor compound comprises a —NR′_{c}—X′₂—N₃ or —NR′_{c}—X-≡CH group, it can react with an alkyne or an azide group of the linker compound to form a triazole group, which is considered to be part of the linker moiety L.

In some embodiments, the linker (L) comprises at least one group selected from the group consisting of: a linear or branched C₁-C₂₀alkylene optionally substituted by one to three groups selected from the group consisting of a C₁-C₆alkyl, a C₃-C₆cycloalkyl, trifluoromethyl, hydroxyl, a halogen, and a C₁-C₆alkoxy; a C₃-C₁₀cycloalkylene; a C₃-C₆heterocycloalkylene; —C(O)—; —O—; —S—; —N(R₁₆)—; —N(R₁₆)—C(O)—; —C(O)—N(R₁₆)—; —CH₂—C(O)—N(R₁₆)—; —N(R₁₆)—C(O)—CH₂—; a polyoxyethylene (PEG) group; an arylene group optionally substituted by one or two groups selected from the group consisting of a C₁-C₆alkyl, trifluoromethyl, hydroxyl, a halogen, and a C₁-C₆alkoxy; and a heteroarylene group, wherein R₁₆ represents hydrogen or C₁-C₆alkyl.

In some embodiments, the linker (L) comprises at least one group selected from: a linear or branched C₁-C₂₀alkylene optionally substituted by one or two groups selected from C₁-C₆alkyl, C₃-C₆cycloalkyl, trifluoromethyl, hydroxyl, halogen, and C₁-C₆alkoxy; a C₃-C₁₀cyclo-alkylene; —C(O)—; —O—; —S—; —N(R₁₆)—; —N(R₁₆)—C(O)—; —C(O)—N(R₁₆)—; —CH₂—C(O)—N(R₁₆)—; —N(R₁₆)—C(O)—CH₂—; a polyoxyethylene (PEG) group; an arylene group optionally substituted by one or two groups selected from C₁-C₆alkyl, trifluoromethyl, hydroxyl, halogen, and C₁-C₆alkoxy; and a heteroarylene group, wherein R₁₆ represents hydrogen or C₁-C₆alkyl.

In some embodiments, the linker (L) comprises a 1,2,3-triazolene group formed by reacting an azide-containing precursor with an alkyne-containing precursor.

In some embodiments, the linker (L) is represented by formula (i):

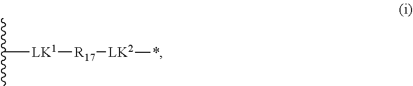

wherein: LK¹ is a bond, —NR¹⁶— or —C(O)—; LK² is a bond, —C(O)— or —N(R₁₆)—C(O)—CH₂—*; R₁₆ is H or methyl; R₁₇ is a C₁-C₂₀alkylene, a C₃₋₁₀cycloalkylene, a C₃₋₁₀cycloalkylene-CH₂—**, phenylene, —C₁-C₂₀alkylene-OCH₂CH₂—**, —C₁-C₂₀alkylene-OCH₂—**, —CH₂—(OCH₂CH₂)ₚ—OCH₂—** or —(CH₂CH₂O)ₚ—(C₁-C₃alkylene)-**, wherein the C₁-C₂₀alkylene or the phenylene is optionally substituted with one or two R₁₇ₐ; and ** indicates the attachment point to LK²; R₁₇ₐ, for each occurrence, is independently a linear or branched C₁-C₆alkyl or a halogen, or two R₁₇ₐ together with the carbon atom from which they are attached form a C₃-C₆cycloalkyl; p is an integer from 1 to 7;

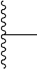

is a bond to the Bcl-xL inhibitor compound; and -* is a bond to DSM.

In some embodiments, the linker (L) is represented by formula (i):

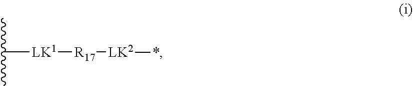

wherein: LK¹ is a bond or —C(O)—; LK² is a bond, —C(O)— or —N(R₁₆)—C(O)—CH₂—*; R₁₆ is H or methyl; R₁₇ is C₁-C₂₀ alkylene, C₃₋₁₀cycloalkylene, phenylene, —CH₂—(OCH₂CH₂)ₚ—OCH₂—, wherein C₁-C₅alkylene or phenylene is optionally substituted with one or two R₁₇ₐ; p is an integer from 1 to 7; R₁₇ₐ, for each occurrence, is independently a linear or branched C₁₋₆alkyl or a halogen, or two R₁₇ₐ together with the carbon atom from which they are attached form a C₃₋₆cycloalkyl, and wherein:

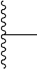

is a bond to the Bcl-xL inhibitor compound; and -* is a bond to DSM.

In some embodiments, the linker (L) is represented by formula (ii):

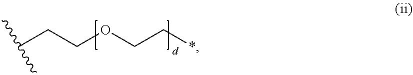

wherein d is an integer from 1 to 7, and wherein:

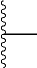

is a bond to the Bcl-xL inhibitor compound; and -* is a bond to DSM.

In some embodiments, the linker (L) is represented by formula (iii):

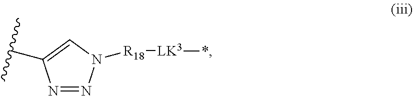

wherein: LK³ is —C(O)— or —N(R₁₆)—C(O)—CH₂—*; R₁₆ is H or methyl; R₁₈ is C₁₋₂₀alkylene or —CH₂CH₂—(OCH₂CH₂)ₚ—**, wherein ** indicates the attachment point to LK³; p is an integer from 1 to 7; and wherein:

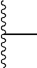

is a bond to the Bcl-xL inhibitor compound; and -* is a bond to DSM.

In some embodiments, the linker (L) is represented by formula (iv):

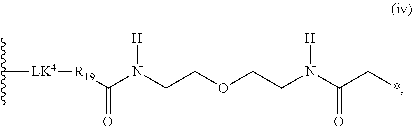

wherein: LK⁴ is a bond or —C(O)—; R₁₉ is C₁₋₆alkylene; and wherein:

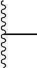

is a bond to the Bcl-xL inhibitor compound; and -* is a bond to DSM.

In some embodiments, the linker (L) is represented by formula (v):

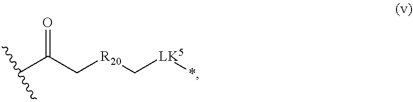

wherein: LK⁵ is a bond or —C(O)—; R₂₀ is C₃₋₁₀cycloalkylene, phenylene, —S— or —N(R₁₆)—; R₁₆ is H or methyl; and wherein:

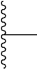

is a bond to the Bcl-xL inhibitor compound; and -* is a bond to DSM.

In some embodiments, the linker (L) is represented by formula (ii):

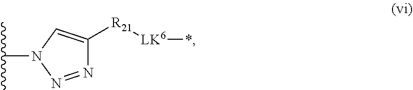

wherein: LK⁶ is a bond, —C(O)—, —O—CH₂—C(O)—*, or —N(R₁₆)—C(O)—CH₂—*; R₁₆ is H or methyl; R₂₁ is C₁₋₂₀alkylene or —CH₂—(OCH₂CH₂)ₚ—**, wherein ** indicates the attachment point to LK⁶; p is an integer from 1 to 7; and wherein:

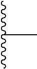

is a bond to the Bcl-xL inhibitor compound; and -* is a bond to DSM. In some embodiments, LK⁶ is a bond, —O—CH₂—C(O)—*, or —N(R₁₆)—C(O)—CH₂—*.

In some embodiments, the linker-(L) is represented by formula (vii):

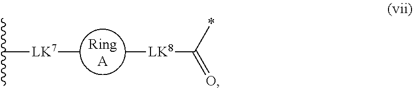

wherein: LK⁷ is a bond or —NR¹⁶—; LK^{a} is a bond, —R₂₂—, —O—R₂₂— or —C(O)—R₂₂—; Ring A is a C₃-C₈ heterocyloalkylene; R₁₆ is H or methyl; R₂₂ is a C₁-C₆alkylene;

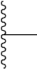

is a bond to the Bcl-xL inhibitor compound; and -* is a bond to DSM.

In some embodiments, L is represented by a formula selected from formulae (L1)-(L109) in Table 3, where

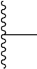

represents a bond to the Bcl-xL inhibitor compound (D), and -* represents a bond to the degradation signaling compound (DSM).

TABLE 3
Exemplary Linkers Showing the Points of Attachment to the Bcl-xL
Inhibitor Compound (D) and the Degradation Signaling Compound
(DSM)

| | |
|---|---|
| L1 | 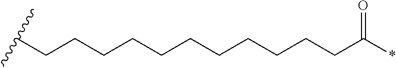 [SMILES: *CCCCCCCCCCCC(*)=O] |
| L2 | 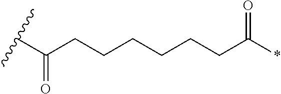 [SMILES: *C(=O)CCCCCCC(*)=O] |
| L3 | 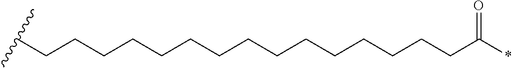 [SMILES: *CCCCCCCCCCCCCCCC(*)=O] |
| L4 | 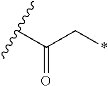 [SMILES: *CC(*)=O] |
| L5 | 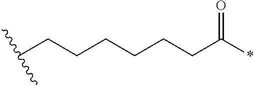 [SMILES: *CCCCCCC(*)=O] |
| L6 | 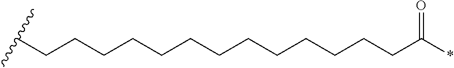 [SMILES: *CCCCCCCCCCCCCC(*)=O] |
| L7 | 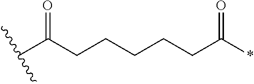 [SMILES: *C(=O)CCCCCC(*)=O] |
| L8 | 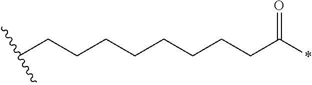 [SMILES: *CCCCCCCCC(*)=O] |
| L9 | 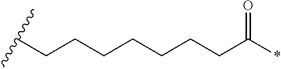 [SMILES: *CCCCCCCC(*)=O] |
| L10 | 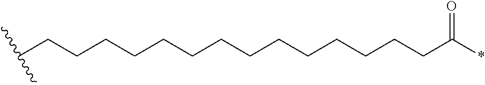 [SMILES: *CCCCCCCCCCCCCCC(*)=O] |
| L11 | 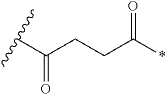 [SMILES: *C(=O)CCC(*)=O] |
| L12 | 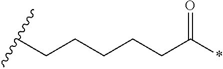 [SMILES: *CCCCCC(*)=O] |
| L13 | 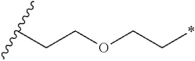 [SMILES: *CCOCC*] |
| L14 | 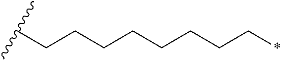 [SMILES: *CCCCCCCC*] |
| L15 | 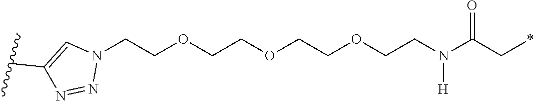 [SMILES: *CC(=O)NCCOCCOCCOCCn1cc(*)nn1] |
| L16 | 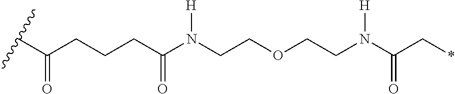 [SMILES: *CC(=O)NCCOCCNC(=O)CCCC(*)=O] |
| L17 | 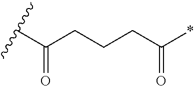 [SMILES: *C(=O)CCCC(*)=O] |
| L18 | 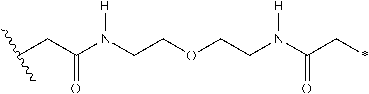 [SMILES: *CC(=O)NCCOCCNC(=O)C*] |
| L19 | 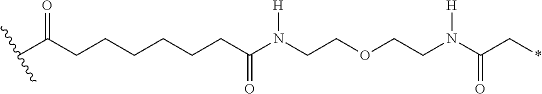 [SMILES: *CC(=O)NCCOCCNC(=O)CCCCCCC(*)=O] |
| L20 | 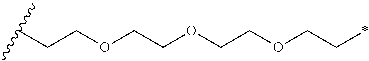 [SMILES: *CCOCCOCCOCC*] |
| L21 | 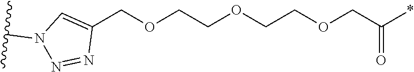 [SMILES: *C(=O)COCCOCCOCc1cn(*)nn1] |
| L22 | 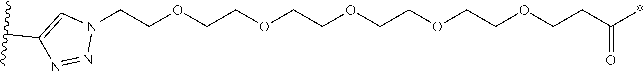 [SMILES: *C(=O)CCOCCOCCOCCOCCOCCn1cc(*)nn1] |
| L23 | 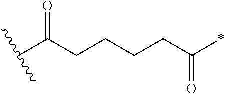 [SMILES: *C(=O)CCCCC(*)=O] |
| L24 | 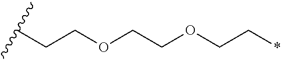 [SMILES: *CCOCCOCC*] |
| L25 | 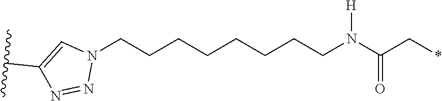 [SMILES: *CC(=O)NCCCCCCCCn1cc(*)nn1] |
| L26 | 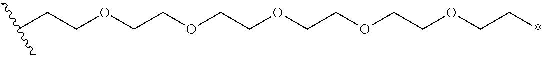 [SMILES: *CCOCCOCCOCCOCCOCC*] |
| L27 | 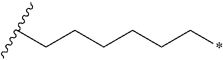 [SMILES: *CCCCCC*] |
| L28 | 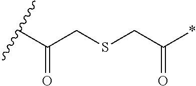 [SMILES: *C(=O)CSCC(*)=O] |
| L29 | 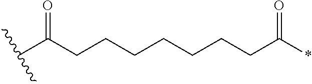 [SMILES: *C(=O)CCCCCCCC(*)=O] |
| L30 | 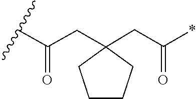 [SMILES: *C(=O)CC1(CC(*)=O)CCCC1] |
| L31 | 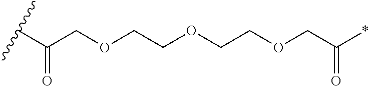 [SMILES: *C(=O)COCCOCCOCC(*)=O] |
| L32 | 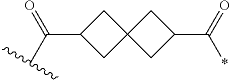 [SMILES: *C(=O)C1CC2(C1)CC(C(*)=O)C2] |
| L33 | 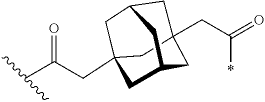 |
| L34 | 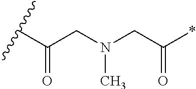 [SMILES: *C(=O)CN(C)CC(*)=O] |
| L35 | 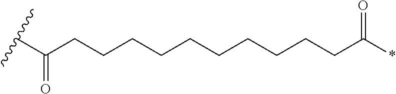 [SMILES: *C(=O)CCCCCCCCCCC(*)=O] |
| L36 | 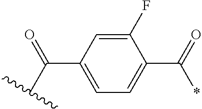 [SMILES: *C(=O)c1ccc(C(*)=O)c(F)c1] |
| L37 | 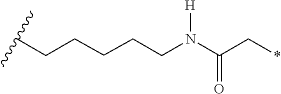 [SMILES: *CCCCCNC(=O)C*] |
| L38 | 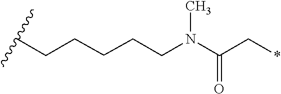 [SMILES: *CCCCCN(C)C(=O)C*] |
| L39 | 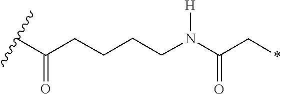 [SMILES: *CC(=O)NCCCCC(*)=O] |
| L40 | 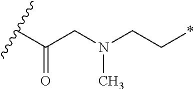 [SMILES: *CCN(C)CC(*)=O] |
| L41 | 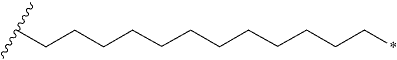 [SMILES: *CCCCCCCCCCCC*] |
| L42 | 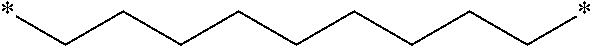 [SMILES: *CCCCCCCCC*] |
| L43 | 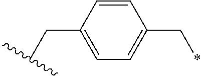 [SMILES: *Cc1ccc(C*)cc1] |
| L44 | 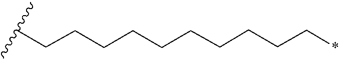 [SMILES: *CCCCCCCCCC*] |
| L45 | 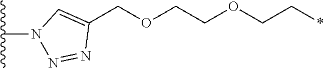 [SMILES: *CCOCCOCc1cn(*)nn1] |
| L46 | 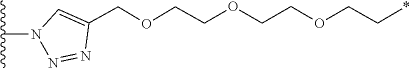 [SMILES: *CCOCCOCCOCc1cn(*)nn1] |
| L47 | 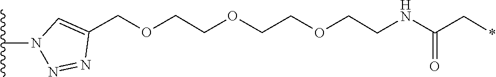 [SMILES: *CC(=O)NCCOCCOCCOCc1cn(*)nn1] |
| L48 | 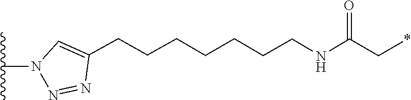 [SMILES: *CC(=O)NCCCCCCCc1cn(*)nn1] |
| L49 | 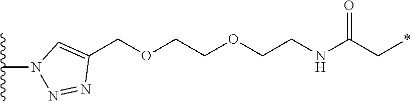 [SMILES: *CC(=O)NCCOCCOCc1cn(*)nn1] |
| L50 | 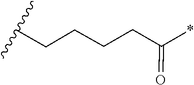 [SMILES: *CCCCC(*)=O] |
| L51 | 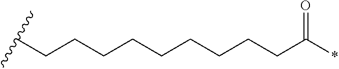 [SMILES: *CCCCCCCCCC(*)=O] |
| L52 | 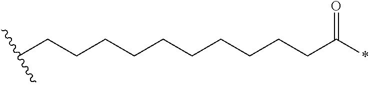 [SMILES: *CCCCCCCCCCC(*)=O] |
| L53 | 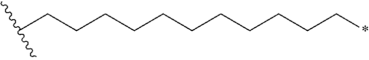 [SMILES: *CCCCCCCCCCC*] |
| L54 | 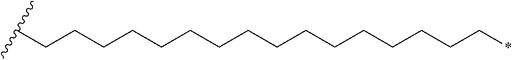 [SMILES: *CCCCCCCCCCCCCCCC*] |
| L55 | 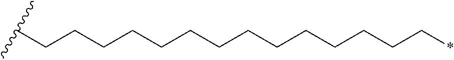 [SMILES: *CCCCCCCCCCCCCC*] |
| L56 | 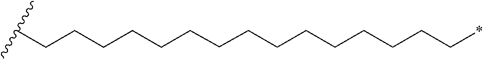 [SMILES: *CCCCCCCCCCCCCCC*] |
| L57 | 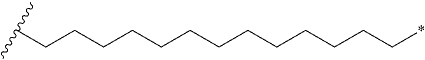 [SMILES: *CCCCCCCCCCCCC*] |
| L58 | 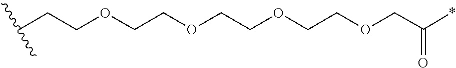 [SMILES: *CCOCCOCCOCCOCC(*)=O] |
| L59 | 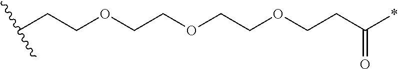 [SMILES: *CCOCCOCCOCCC(*)=O] |
| L60 | 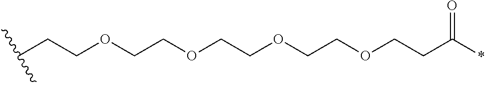 [SMILES: *CCOCCOCCOCCOCCC(*)=O] |
| L61 | 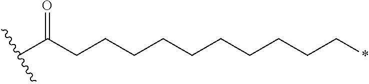 [SMILES: *CCCCCCCCCCC(*)=O] |
| L62 | 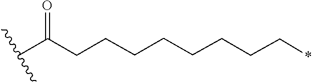 [SMILES: *CCCCCCCCC(*)=O] |
| L63 | 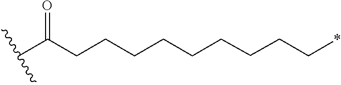 [SMILES: *CCCCCCCCCC(*)=O] |
| L64 | 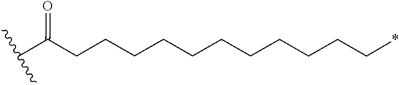 [SMILES: *CCCCCCCCCCCC(*)=O] |
| L65 | 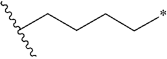 [SMILES: *CCCC*] |
| L66 | 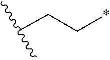 [SMILES: *CC*] |
| L67 | 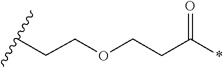 [SMILES: *CCOCCC(*)=O] |
| L68 | 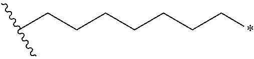 [SMILES: *CCCCCCC*] |
| L69 | 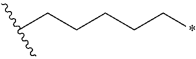 [SMILES: *CCCCC*] |
| L70 | 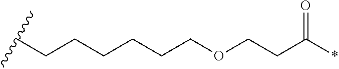 [SMILES: *CCCCCCOCCC(*)=O] |
| L71 | 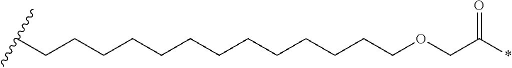 [SMILES: *CCCCCCCCCCCCCOCC(*)=O] |
| L72 | 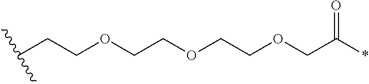 [SMILES: *CCOCCOCCOCC(*)=O] |
| L73 | 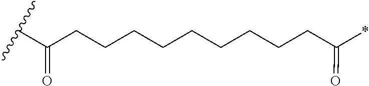 [SMILES: *C(=O)CCCCCCCCCC(*)=O] |
| L74 | 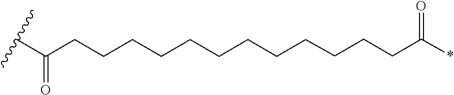 [SMILES: *C(=O)CCCCCCCCCCCCC(*)=O] |
| L75 | 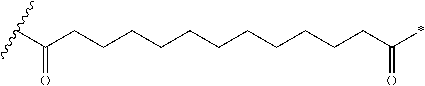 [SMILES: *C(=O)CCCCCCCCCCCC(*)=O] |
| L76 | 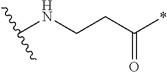 [SMILES: *NCCC(*)=O] |
| L77 | 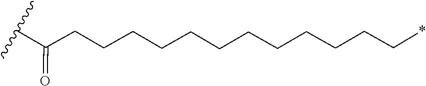 [SMILES: *CCCCCCCCCCCCC(*)=O] |
| L78 | 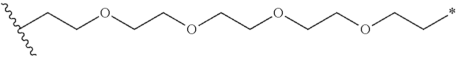 [SMILES: *CCOCCOCCOCCOCC*] |
| L79 | 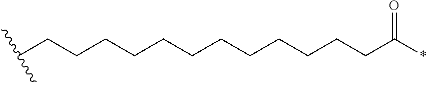 [SMILES: *CCCCCCCCCCCCC(*)=O] |
| L80 | 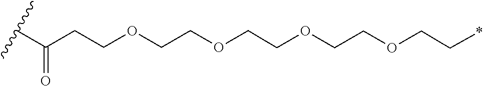 [SMILES: *CCOCCOCCOCCOCCC(*)=O] |
| L81 | 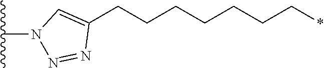 [SMILES: *CCCCCCCc1cn(*)nn1] |
| L82 | 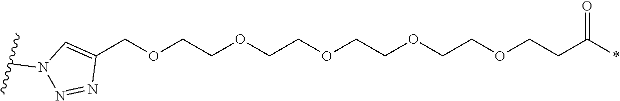 [SMILES: *C(=O)CCOCCOCCOCCOCCOCc1cn(*)nn1] |
| L83 | 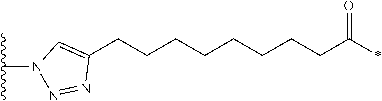 [SMILES: *C(=O)CCCCCCCCc1cn(*)nn1] |
| L84 | 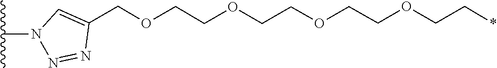 [SMILES: *CCOCCOCCOCCOCc1cn(*)nn1] |
| L85 | 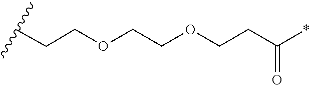 [SMILES: *CCOCCOCCC(*)=O] |
| L86 | 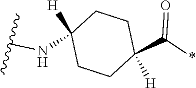 [SMILES: *N[C@H]1CC[C@H](C(*)=O)CC1] |
| L87 | 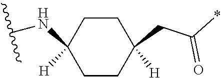 [SMILES: *N[C@H]1CC[C@@H](CC(*)=O)CC1] |
| L88 | 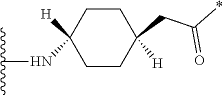 [SMILES: *N[C@H]1CC[C@H](CC(*)=O)CC1] |
| L89 | 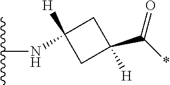 [SMILES: *N[C@H]1C[C@H](C(*)=O)C1] |
| L90 | 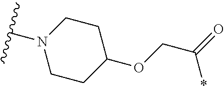 [SMILES: *C(=O)COC1CCN(*)CC1] |
| L91 | 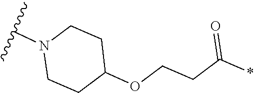 [SMILES: *C(=O)CCOC1CCN(*)CC1] |
| L92 | 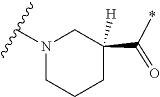 [SMILES: *C(=O)[C@H]1CCCN(*)C1] |
| L93 | 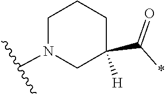 [SMILES: *C(=O)[C@@H]1CCCN(*)C1] |
| L94 | 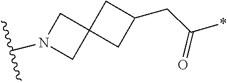 [SMILES: *C(=O)CC1CC2(C1)CN(*)C2] |
| L95 | 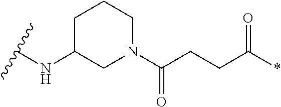 [SMILES: *NC1CCCN(C(=O)CCC(*)=O)C1] |
| L96 | 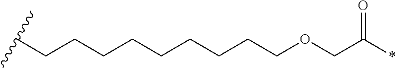 [SMILES: *CCCCCCCCCOCC(*)=O] |
| L97 | 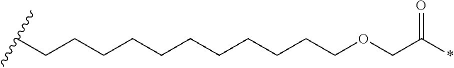 [SMILES: *CCCCCCCCCCCOCC(*)=O] |
| L98 | 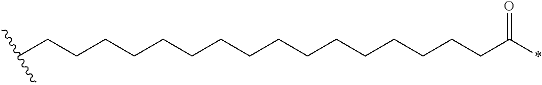 [SMILES: *CCCCCCCCCCCCCCCCC(*)=O] |
| L99 | 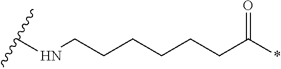 [SMILES: *NCCCCCCC(*)=O] |
| L100 | 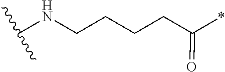 [SMILES: *NCCCCC(*)=O] |
| L101 | 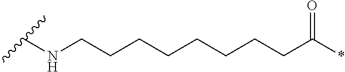 [SMILES: *NCCCCCCCCC(*)=O] |
| L102 | 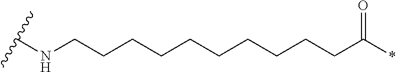 [SMILES: *NCCCCCCCCCCC(*)=O] |
| L103 | 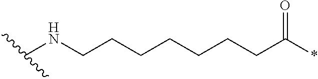 [SMILES: *NCCCCCCCC(*)=O] |
| L104 | 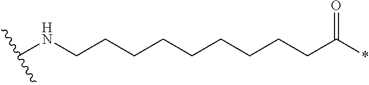 [SMILES: *NCCCCCCCCCC(*)=O] |
| L105 | 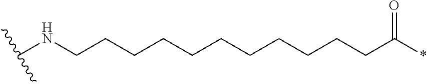 [SMILES: *NCCCCCCCCCCCC(*)=O] |
| L106 | 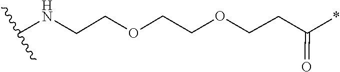 [SMILES: *NCCOCCOCCC(*)=O] |
| L107 | 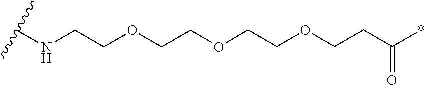 [SMILES: *NCCOCCOCCOCCC(*)=O] |
| L108 | 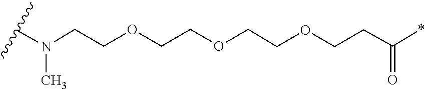 [SMILES: *C(=O)CCOCCOCCOCCN(*)C] |
| L109 | 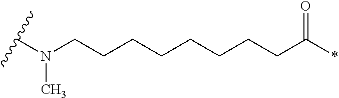 [SMILES: *C(=O)CCCCCCCCN(*)C] |

In some embodiments, D-L in Formula (A) is represented by a formula in Table 4, or an enantiomer, diastereoisomer and/or a pharmaceutically acceptable salt thereof, where -* represents a bond to the degradation signaling compound (DSM).

TABLE 4
Exemplary D-L Moieties Showing the Point of Attachement to the
Degradation Signaling Compound (DSM)

| | |
|---|---|
| D1a- L1 | 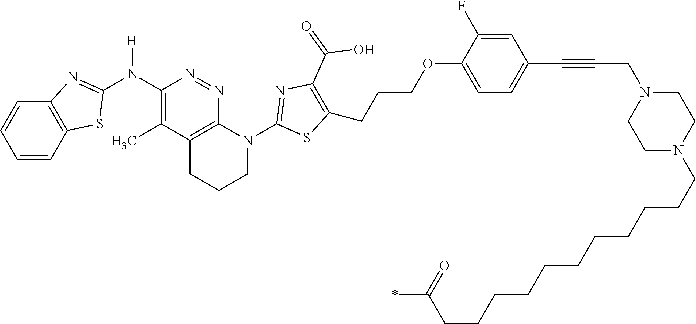 [SMILES: *C(=O)CCCCCCCCCCCN1CCN(CC#Cc2ccc(OCCCc3sc(N4CCCc5c4nnc(Nc4nc6ccccc6s4)c5C)nc3C(=O)O)c(F)c2)CC1] |
| D1a- L2 | 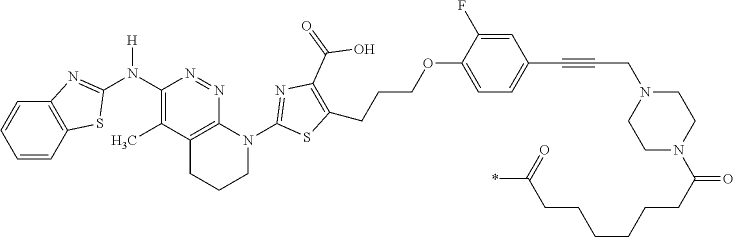 [SMILES: *C(=O)CCCCCCC(=O)N1CCN(CC#Cc2ccc(OCCCc3sc(N4CCCc5c4nnc(Nc4nc6ccccc6s4)c5C)nc3C(=O)O)c(F)c2)CC1] |
| D1a- L7 | 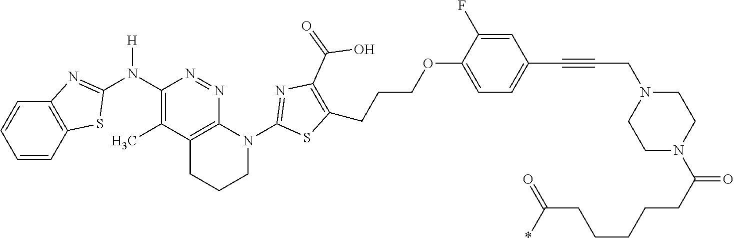 [SMILES: *C(=O)CCCCCC(=O)N1CCN(CC#Cc2ccc(OCCCc3sc(N4CCCc5c4nnc(Nc4nc6ccccc6s4)c5C)nc3C(=O)O)c(F)c2)CC1] |
| D2a- L3 | 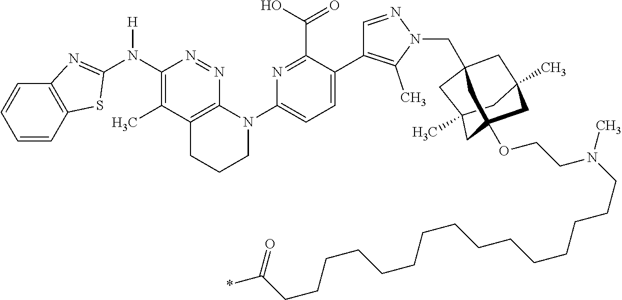 |
| D2a- L1 | 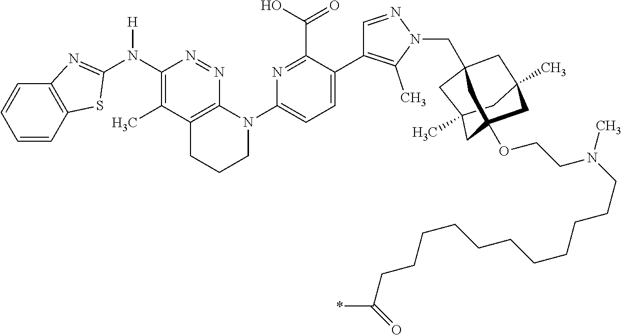 |
| D2a- L5 | 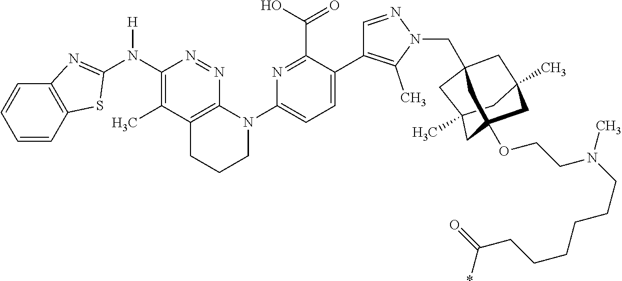 |
| D2a- L6 | 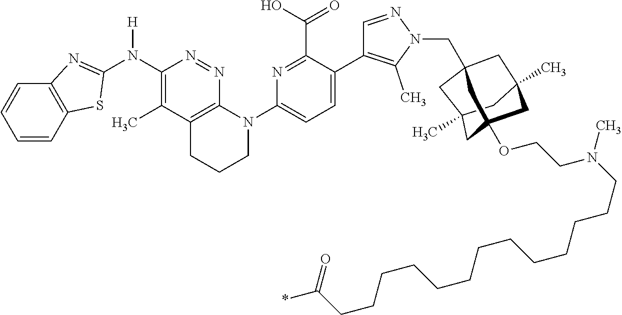 |
| D2a- L8 | 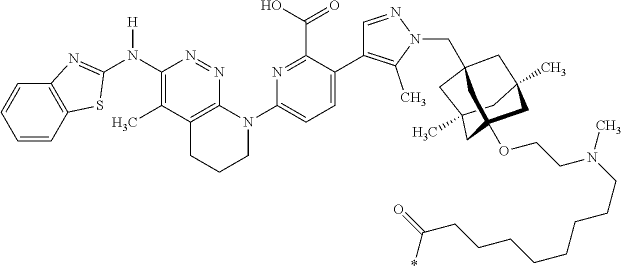 |
| D2a- L9 | 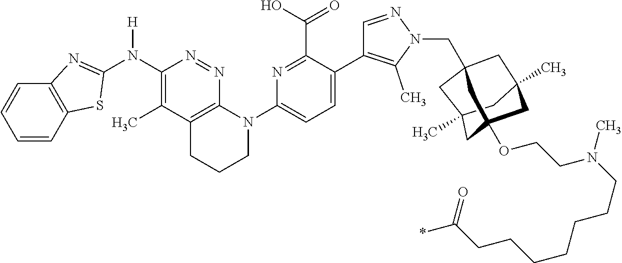 |
| D2a- L10 | 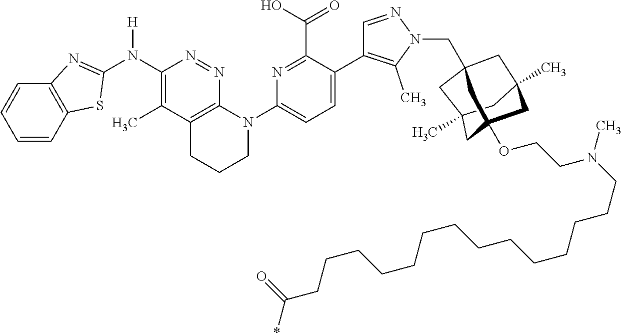 |
| D1a- L11 | 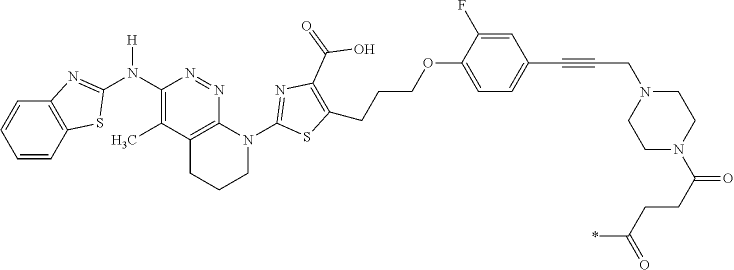 [SMILES: *C(=O)CCC(=O)N1CCN(CC#Cc2ccc(OCCCc3sc(N4CCCc5c4nnc(Nc4nc6ccccc6s4)c5C)nc3C(=O)O)c(F)c2)CC1] |
| D1a- L12 | 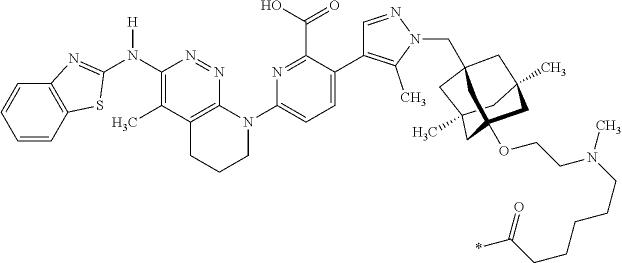 |
| D2a- L13 | 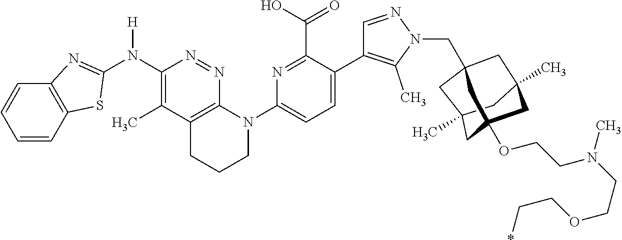 |
| D2a- L14 | 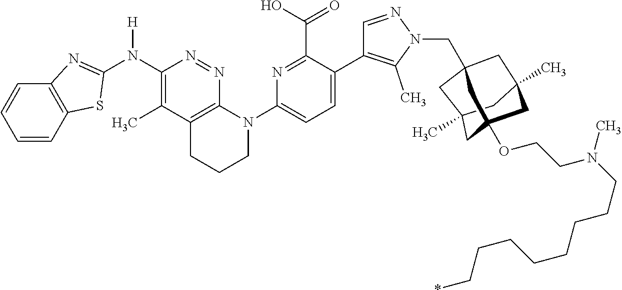 |
| D3a- L15 | 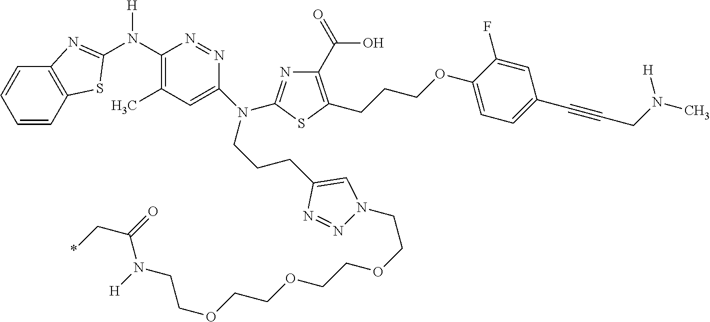 [SMILES: *CC(=O)NCCOCCOCCOCCn1cc(CCCN(c2cc(C)c(Nc3nc4ccccc4s3)nn2)c2nc(C(=O)O)c(CCCOc3ccc(C#CCNC)cc3F)s2)nn1] |
| D1a- L16 | 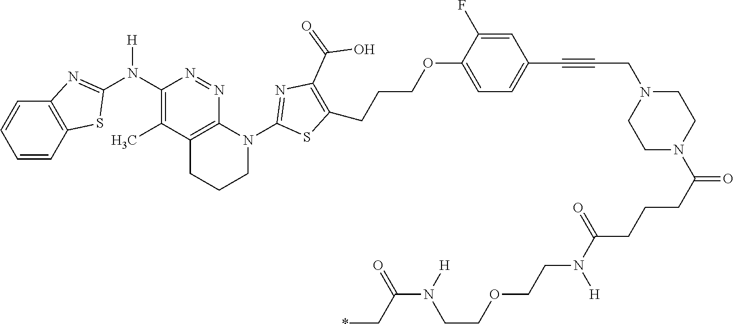 [SMILES: *CC(=O)NCCOCCNC(=O)CCCC(=O)N1CCN(CC#Cc2ccc(OCCCc3sc(N4CCCc5c4nnc(Nc4nc6ccccc6s4)c5C)nc3C(=O)O)c(F)c2)CC1] |
| D1a- L17 | 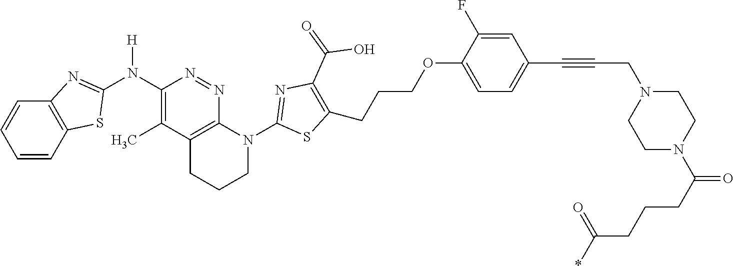 [SMILES: *C(=O)CCCC(=O)N1CCN(CC#Cc2ccc(OCCCc3sc(N4CCCc5c4nnc(Nc4nc6ccccc6s4)c5C)nc3C(=O)O)c(F)c2)CC1] |
| D4a- L18 | 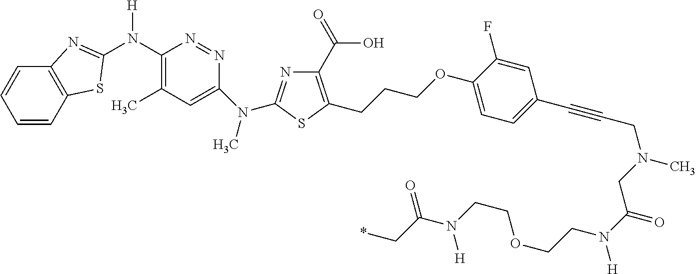 [SMILES: *CC(=O)NCCOCCNC(=O)CN(C)CC#Cc1ccc(OCCCc2sc(N(C)c3cc(C)c(Nc4nc5ccccc5s4)nn3)nc2C(=O)O)c(F)c1] |
| D6a- L7 | 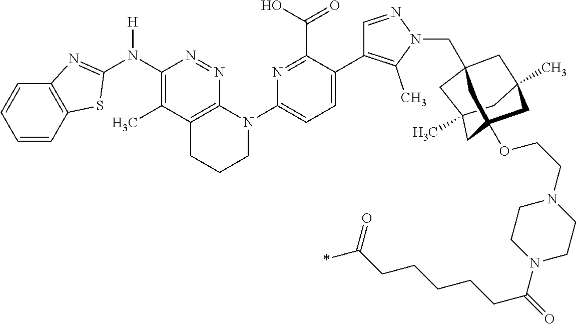 |
| D1a- L19 | 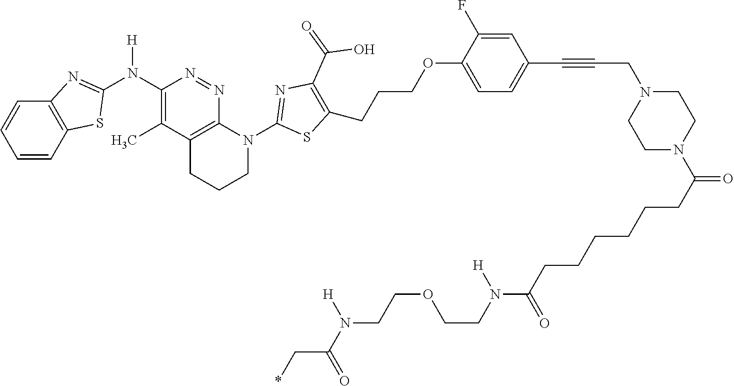 [SMILES: *CC(=O)NCCOCCNC(=O)CCCCCCC(=O)N1CCN(CC#Cc2ccc(OCCCc3sc(N4CCCc5c4nnc(Nc4nc6ccccc6s4)c5C)nc3C(=O)O)c(F)c2)CC1] |
| D2a- L20 | 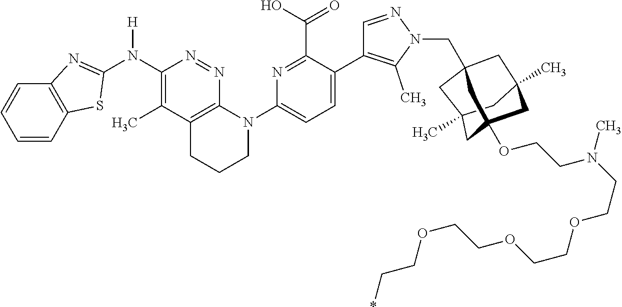 |
| D5a- L21 | 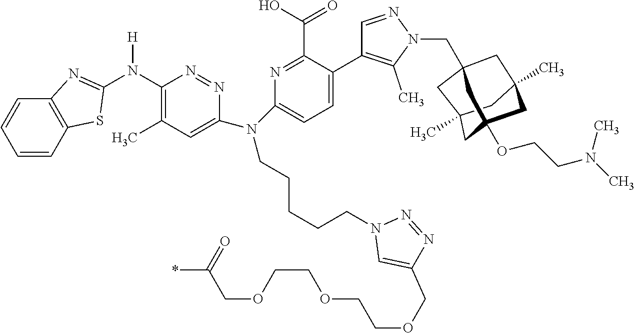 |
| D3a- L22 | 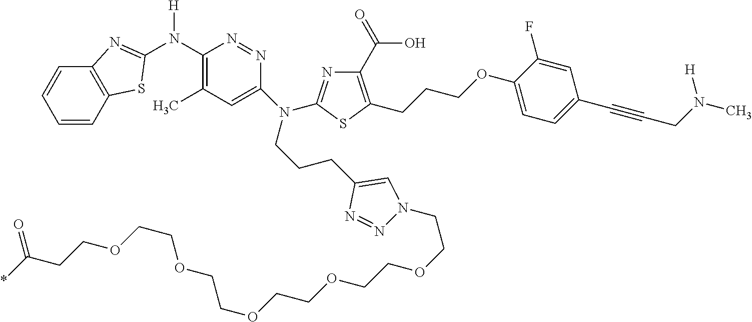 [SMILES: *C(=O)CCOCCOCCOCCOCCOCCn1cc(CCCN(c2cc(C)c(Nc3nc4ccccc4s3)nn2)c2nc(C(=O)O)c(CCCOc3ccc(C#CCNC)cc3F)s2)nn1] |
| D6a- L23 | 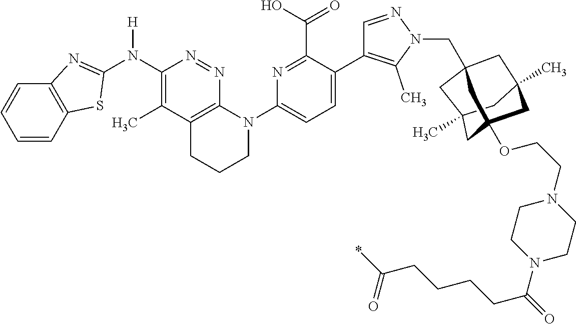 |
| D2a- L24 | 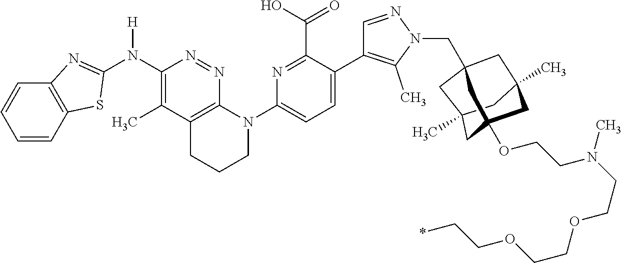 |
| D3a- L25 | 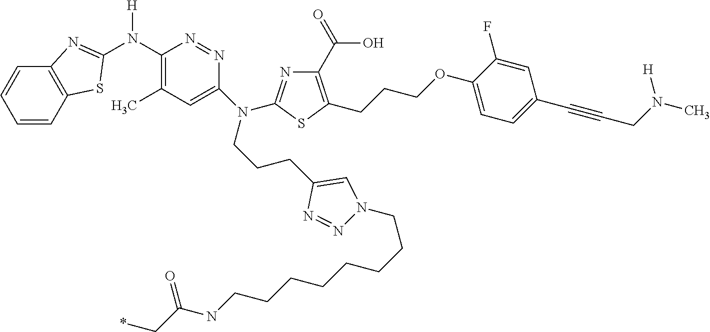 [SMILES: *CC(=O)NCCCCCCCCn1cc(CCCN(c2cc(C)c(Nc3nc4ccccc4s3)nn2)c2nc(C(=O)O)c(CCCOc3ccc(C#CCNC)cc3F)s2)nn1] |
| D2a- L26 | 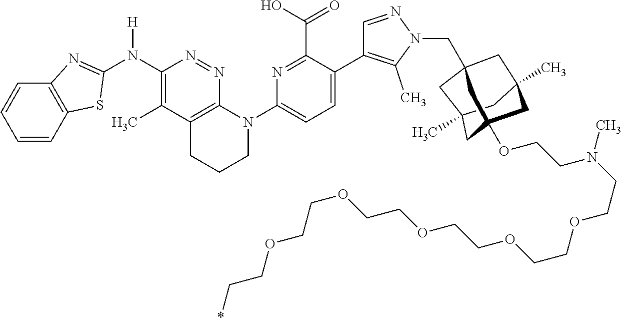 |
| D2a- L27 | 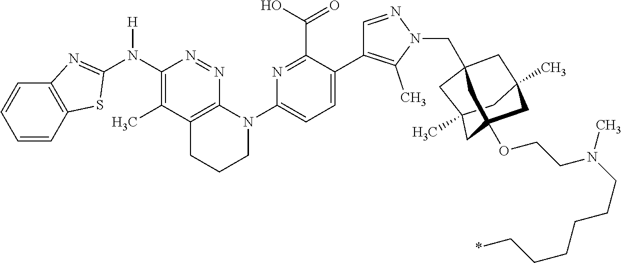 |
| D1a- L23 | 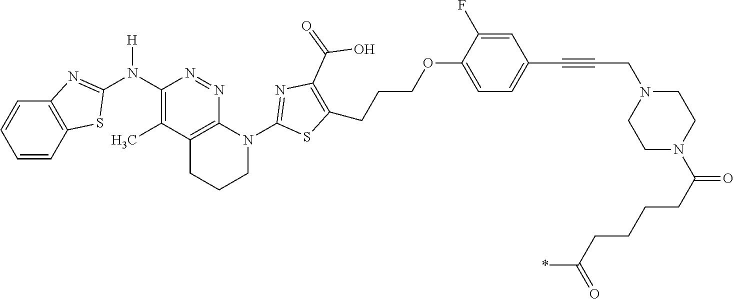 [SMILES: *C(=O)CCCCC(=O)N1CCN(CC#Cc2ccc(OCCCc3sc(N4CCCc5c4nnc(Nc4nc6ccccc6s4)c5C)nc3C(=O)O)c(F)c2)CC1] |
| D1a- L28 | 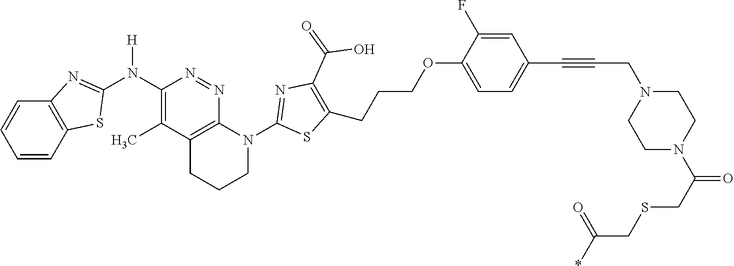 [SMILES: *C(=O)CSCC(=O)N1CCN(CC#Cc2ccc(OCCCc3sc(N4CCCc5c4nnc(Nc4nc6ccccc6s4)c5C)nc3C(=O)O)c(F)c2)CC1] |
| D1a- L29 | 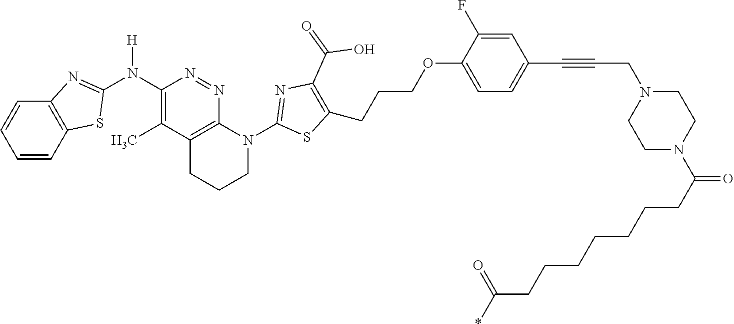 [SMILES: *C(=O)CCCCCCCC(=O)N1CCN(CC#Cc2ccc(OCCCc3sc(N4CCCc5c4nnc(Nc4nc6ccccc6s4)c5C)nc3C(=O)O)c(F)c2)CC1] |
| D1a- L30 | 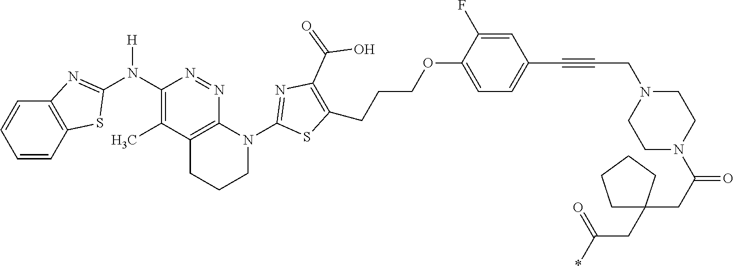 [SMILES: *C(=O)CC1(CC(=O)N2CCN(CC#Cc3ccc(OCCCc4sc(N5CCCc6c5nnc(Nc5nc7ccccc7s5)c6C)nc4C(=O)O)c(F)c3)CC2)CCCC1] |
| D1a- L31 | 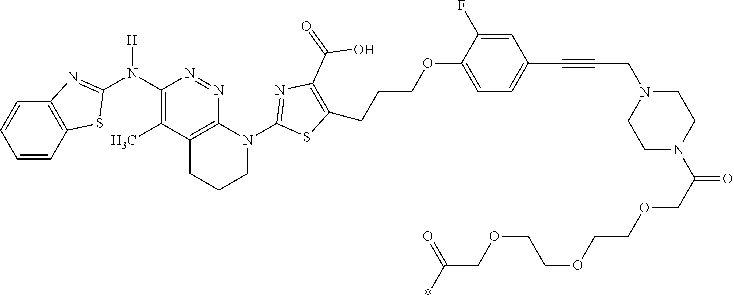 [SMILES: *C(=O)COCCOCCOCC(=O)N1CCN(CC#Cc2ccc(OCCCc3sc(N4CCCc5c4nnc(Nc4nc6ccccc6s4)c5C)nc3C(=O)O)c(F)c2)CC1] |
| D1a- L32 | 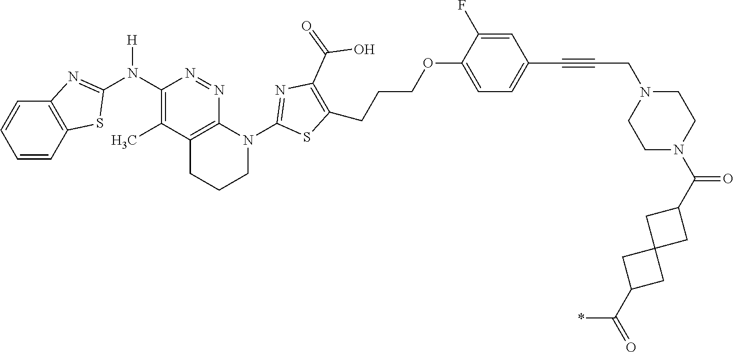 [SMILES: *C(=O)C1CC2(C1)CC(C(=O)N1CCN(CC#Cc3ccc(OCCCc4sc(N5CCCc6c5nnc(Nc5nc7ccccc7s5)c6C)nc4C(=O)O)c(F)c3)CC1)C2] |
| D1a- L33 | 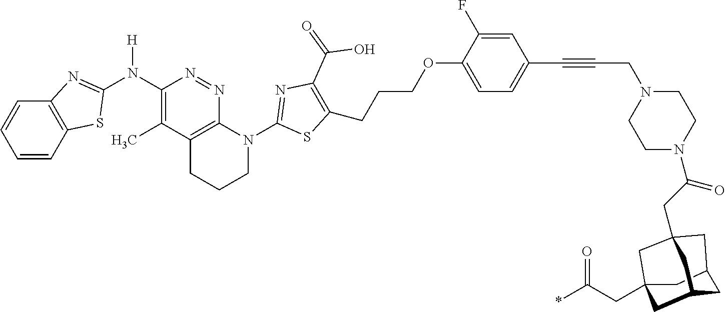 |
| D1a- L34 | 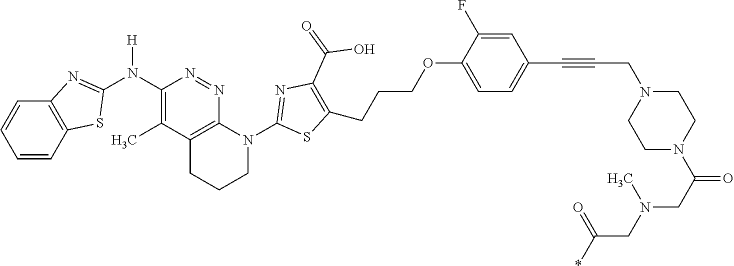 [SMILES: *C(=O)CN(C)CC(=O)N1CCN(CC#Cc2ccc(OCCCc3sc(N4CCCc5c4nnc(Nc4nc6ccccc6s4)c5C)nc3C(=O)O)c(F)c2)CC1] |
| D1a- L35 | 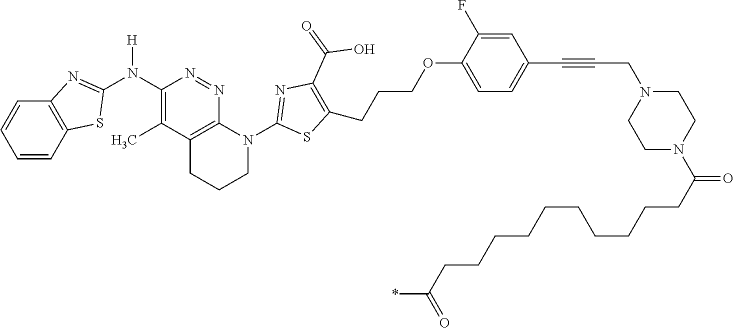 [SMILES: *C(=O)CCCCCCCCCCC(=O)N1CCN(CC#Cc2ccc(OCCCc3sc(N4CCCc5c4nnc(Nc4nc6ccccc6s4)c5C)nc3C(=O)O)c(F)c2)CC1] |
| D1a- L36 | 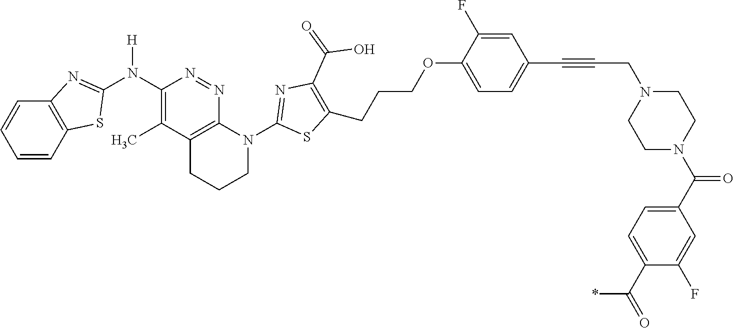 [SMILES: *C(=O)c1ccc(C(=O)N2CCN(CC#Cc3ccc(OCCCc4sc(N5CCCc6c5nnc(Nc5nc7ccccc7s5)c6C)nc4C(=O)O)c(F)c3)CC2)cc1F] |
| D1a- L8 | 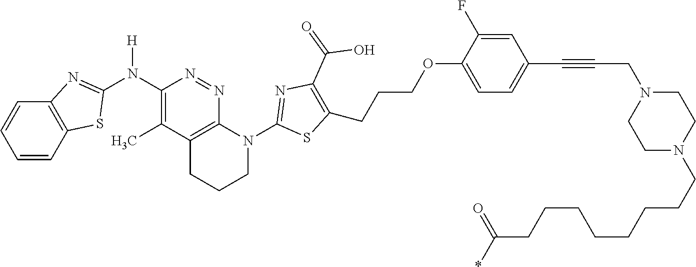 [SMILES: *C(=O)CCCCCCCCN1CCN(CC#Cc2ccc(OCCCc3sc(N4CCCc5c4nnc(Nc4nc6ccccc6s4)c5C)nc3C(=O)O)c(F)c2)CC1] |
| D1a- L37 | 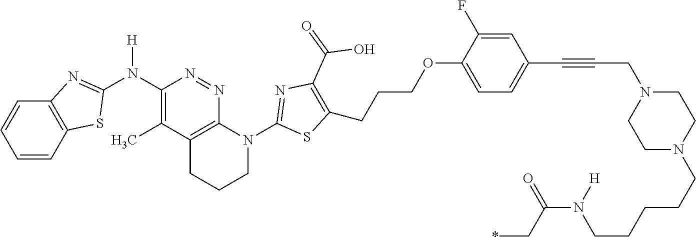 [SMILES: *CC(=O)NCCCCCN1CCN(CC#Cc2ccc(OCCCc3sc(N4CCCc5c4nnc(Nc4nc6ccccc6s4)c5C)nc3C(=O)O)c(F)c2)CC1] |
| D1- L38 | 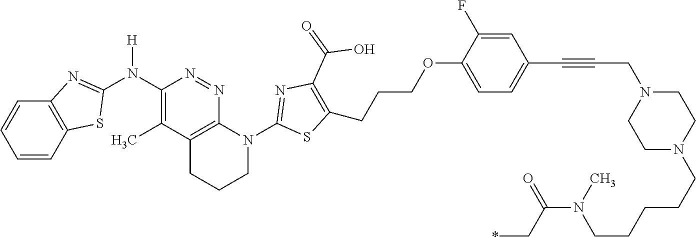 [SMILES: *CC(=O)N(C)CCCCCN1CCN(CC#Cc2ccc(OCCCc3sc(N4CCCc5c4nnc(Nc4nc6ccccc6s4)c5C)nc3C(=O)O)c(F)c2)CC1] |
| D1a- L39 | 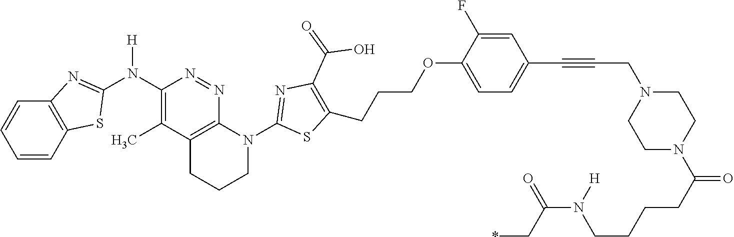 [SMILES: *CC(=O)NCCCCC(=O)N1CCN(CC#Cc2ccc(OCCCc3sc(N4CCCc5c4nnc(Nc4nc6ccccc6s4)c5C)nc3C(=O)O)c(F)c2)CC1] |
| D1a- L40 | 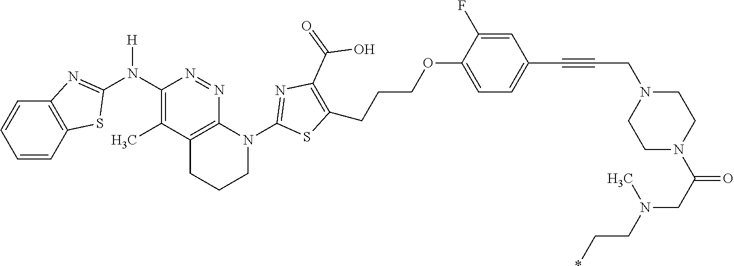 [SMILES: *CCN(C)CC(=O)N1CCN(CC#Cc2ccc(OCCCc3sc(N4CCCc5c4nnc(Nc4nc6ccccc6s4)c5C)nc3C(=O)O)c(F)c2)CC1] |
| D1a- L41 | 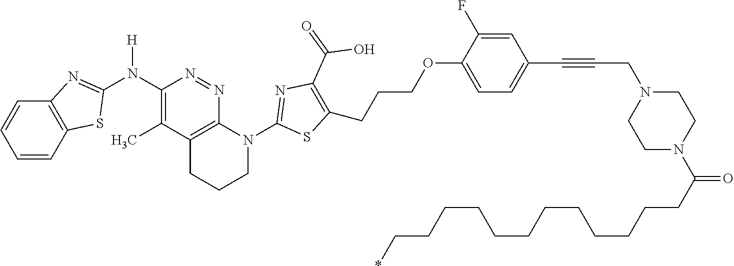 [SMILES: *CCCCCCCCCCCCC(=O)N1CCN(CC#Cc2ccc(OCCCc3sc(N4CCCc5c4nnc(Nc4nc6ccccc6s4)c5C)nc3C(=O)O)c(F)c2)CC1] |
| D6a- L11 | 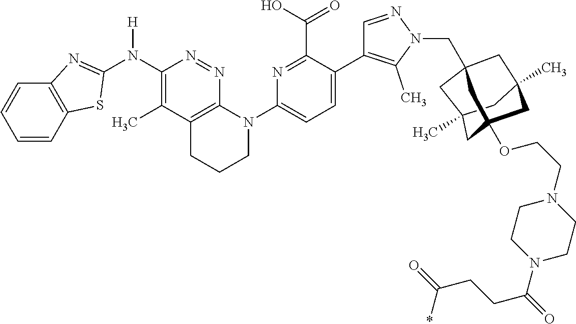 |
| D6a- L4 | 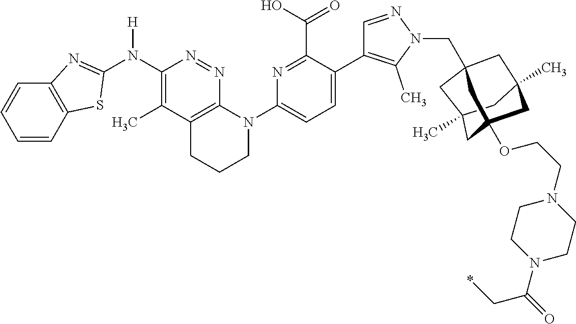 |
| D6a- L2 | 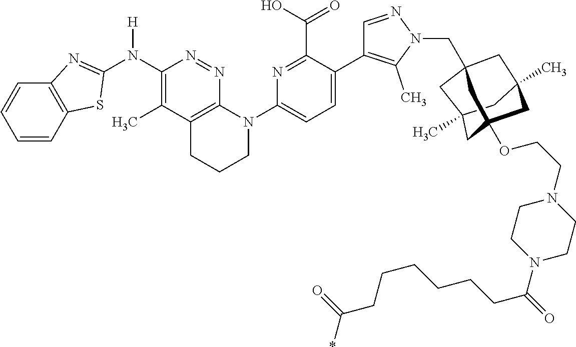 |
| D6a- L35 | 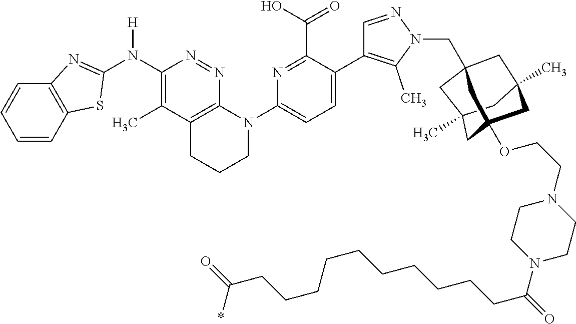 |
| D1a- L14 | 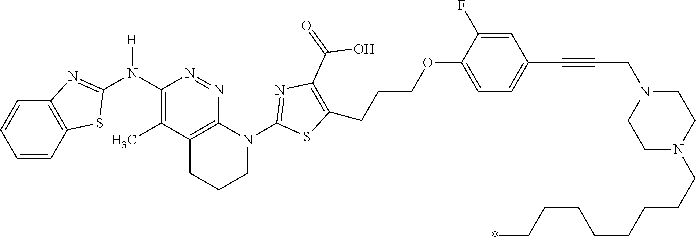 [SMILES: *CCCCCCCCN1CCN(CC#Cc2ccc(OCCCc3sc(N4CCCc5c4nnc(Nc4nc6ccccc6s4)c5C)nc3C(=O)O)c(F)c2)CC1] |
| D1a- L42 | 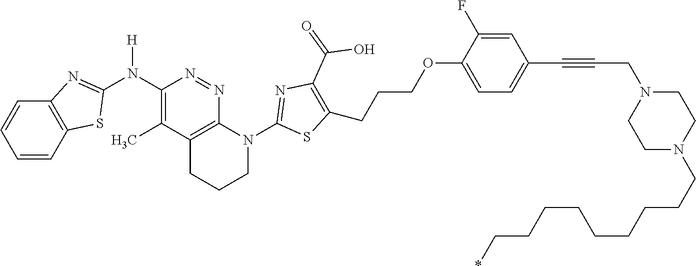 [SMILES: *CCCCCCCCCN1CCN(CC#Cc2ccc(OCCCc3sc(N4CCCc5c4nnc(Nc4nc6ccccc6s4)c5C)nc3C(=O)O)c(F)c2)CC1] |
| D1a- L43 | 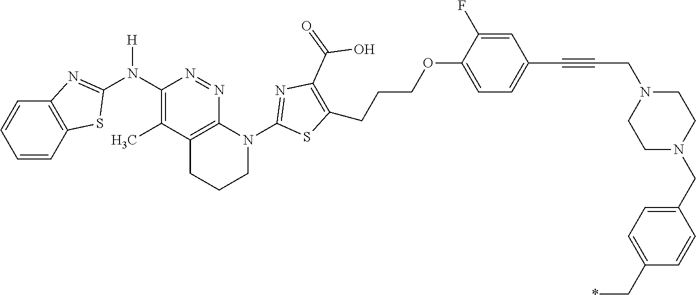 [SMILES: *Cc1ccc(CN2CCN(CC#Cc3ccc(OCCCc4sc(N5CCCc6c5nnc(Nc5nc7ccccc7s5)c6C)nc4C(=O)O)c(F)c3)CC2)cc1] |
| D2a- L44 | 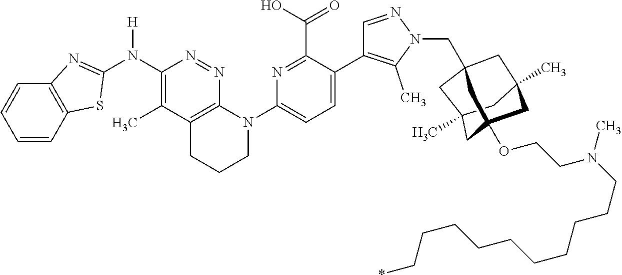 |
| D5a- L45 | 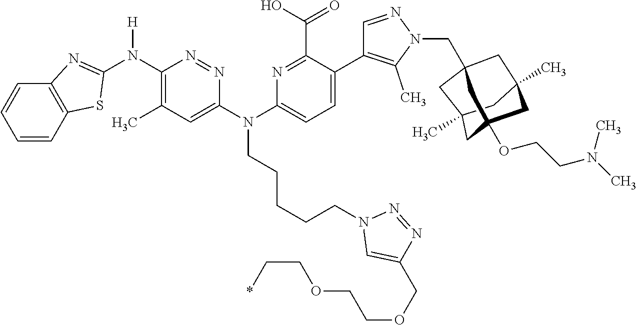 |
| D5a- L46 | 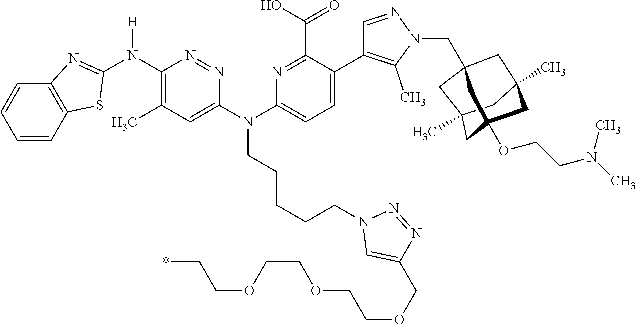 |
| D5a- L47 | 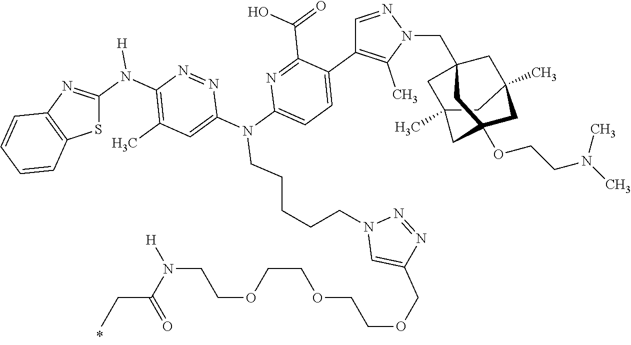 |
| D5a- L48 | 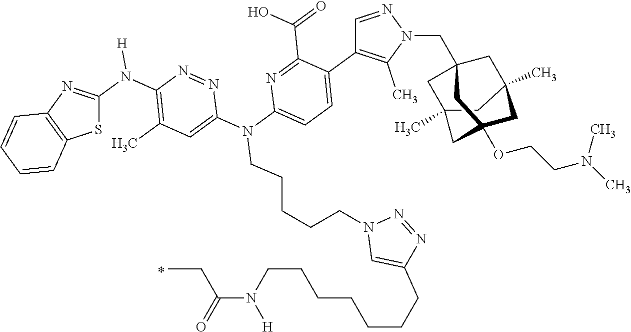 |
| D5a- L49 | 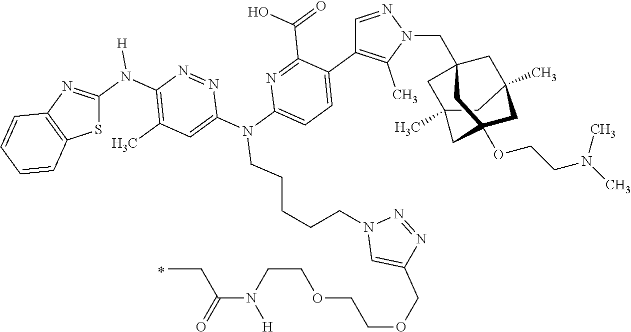 |
| D7a- L50 | 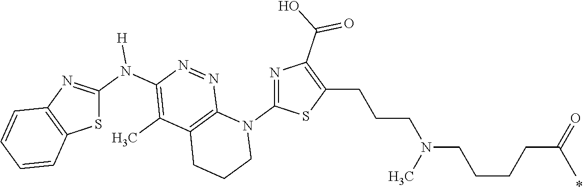 [SMILES: CC(=O)CCCCN(C)CCCc1sc(N2CCCc3c2nnc(Nc2nc4ccccc4s2)c3C)nc1C(=O)O] |
| D2a- L51 | 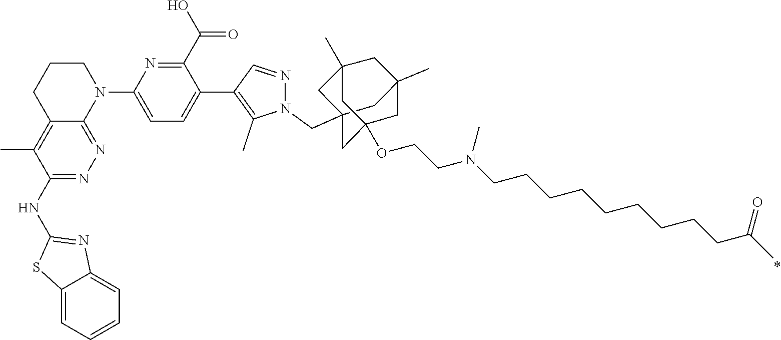 [SMILES: CC(=O)CCCCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D2a- L42 | 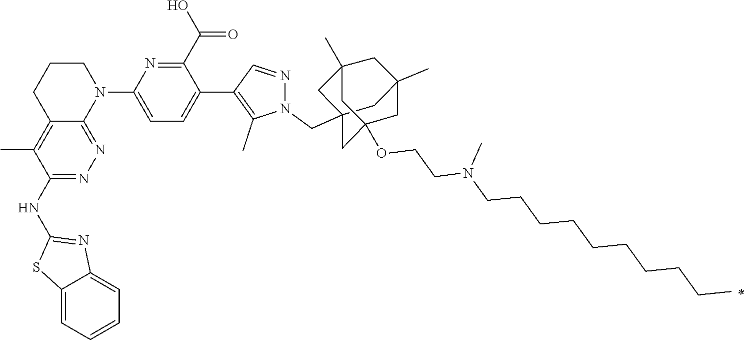 [SMILES: CCCCCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D2a- L52 | 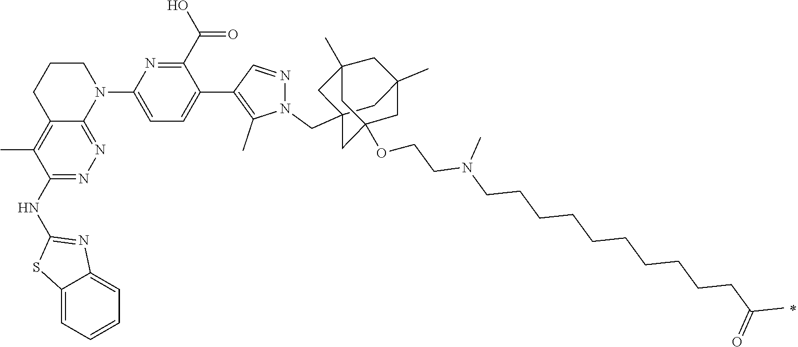 [SMILES: CC(=O)CCCCCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D2a- L53 | 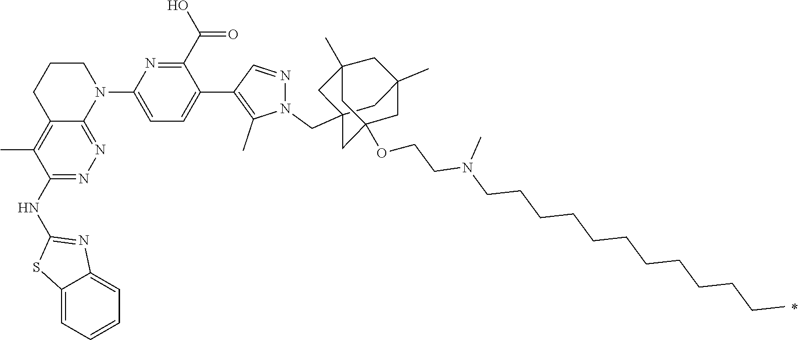 [SMILES: CCCCCCCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D2a- L54 | 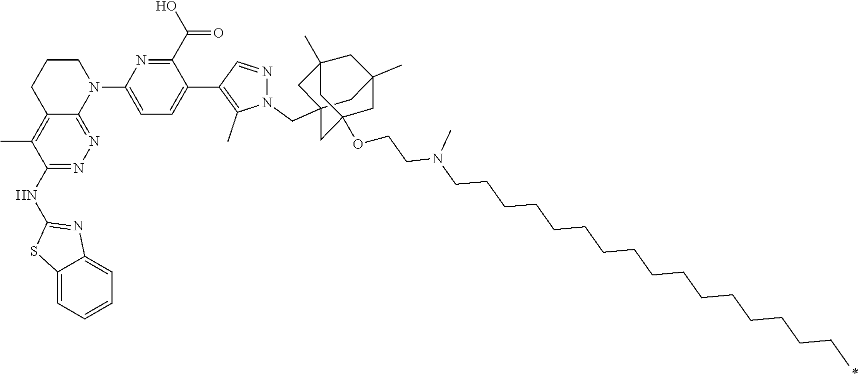 [SMILES: CCCCCCCCCCCCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D2a- L79 | 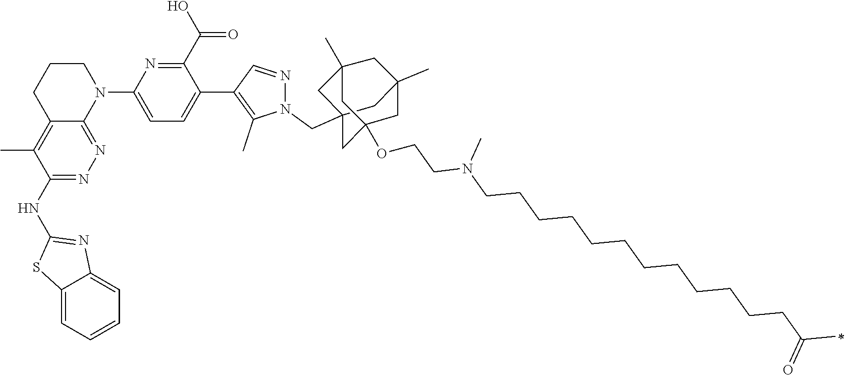 [SMILES: CC(=O)CCCCCCCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D2a- L41 | 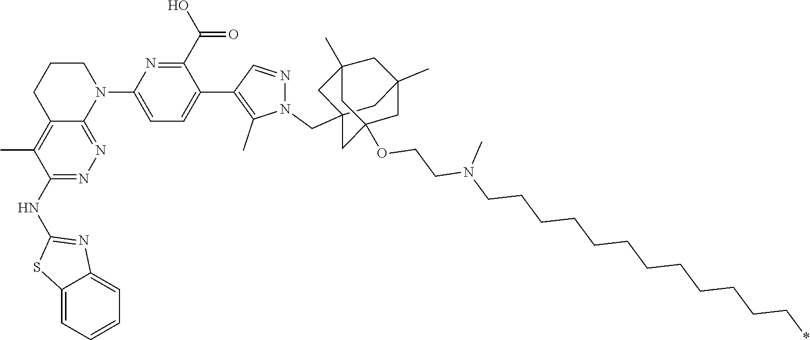 [SMILES: *CCCCCCCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D2a- L55 | 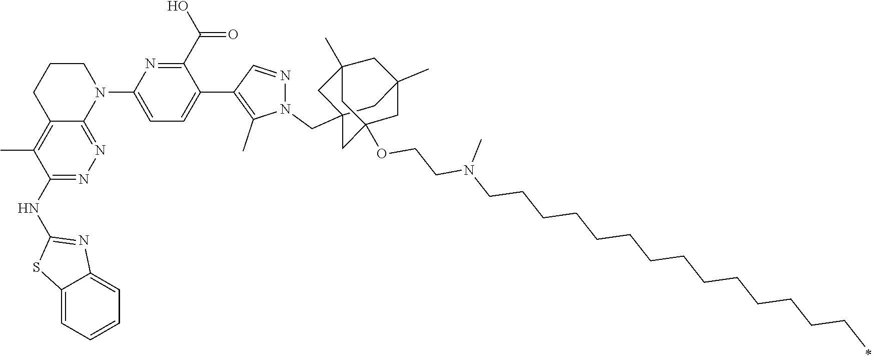 [SMILES: CCCCCCCCCCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D2a- L56 | 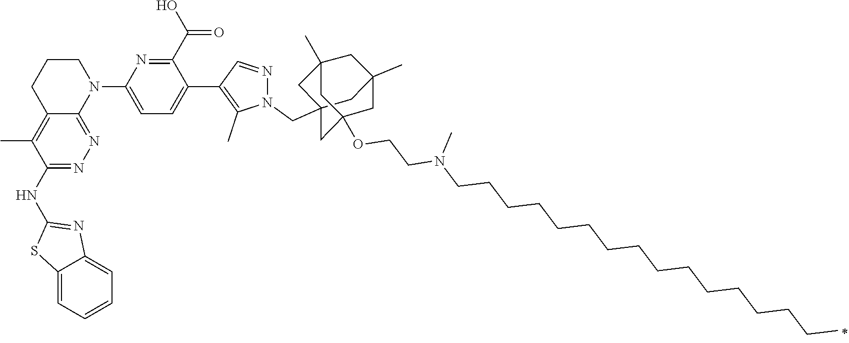 [SMILES: CCCCCCCCCCCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D2a- L57 | 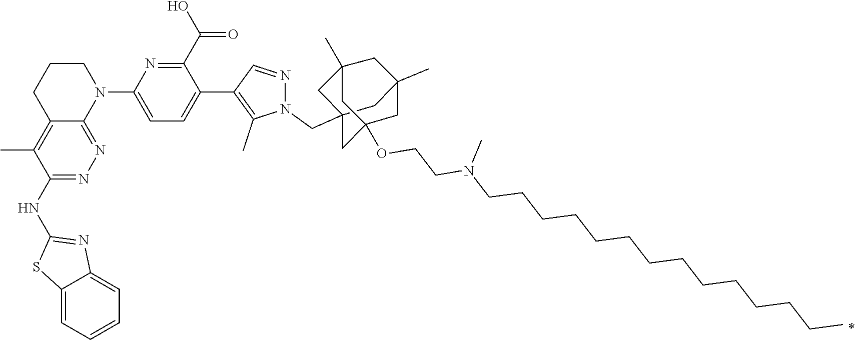 [SMILES: CCCCCCCCCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D2a- L58 | 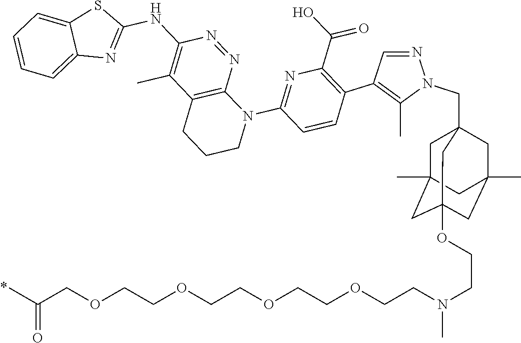 [SMILES: CC(=O)COCCOCCOCCOCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D2a- L59 | 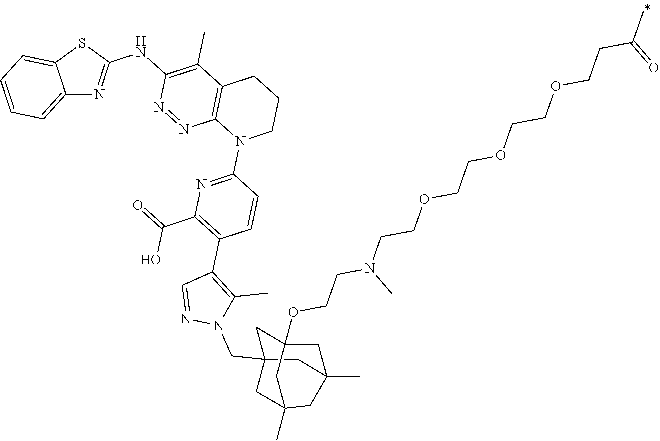 [SMILES: CC(=O)CCOCCOCCOCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D2a- L60 | 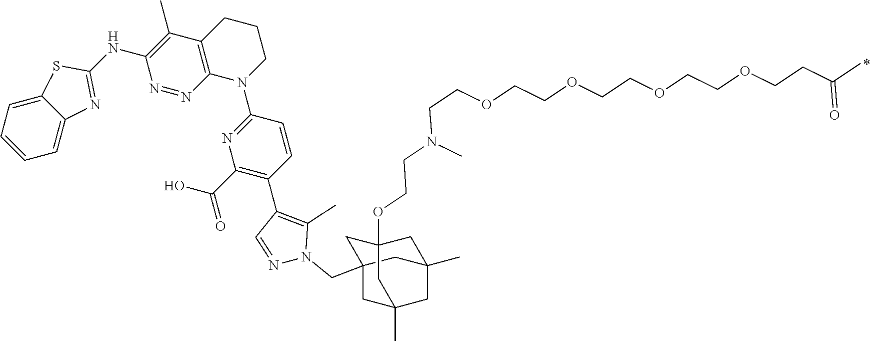 [SMILES: CC(=O)CCOCCOCCOCCOCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D8a- L61 | 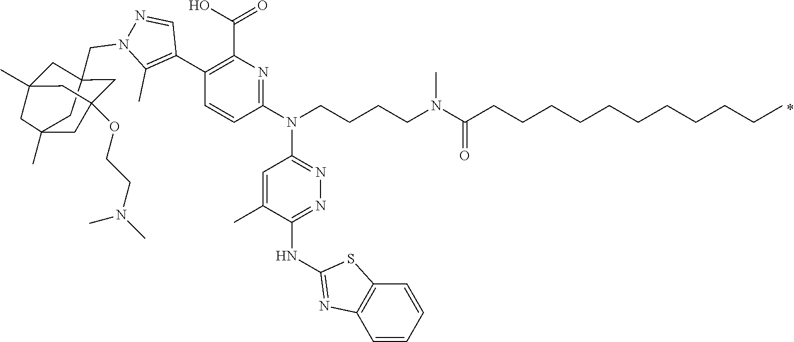 [SMILES: CCCCCCCCCCCC(=O)N(C)CCCCN(c1cc(C)c(Nc2nc3ccccc3s2)nn1)c1ccc(-c2cnn(CC34CC5(C)CC(C)(C3)CC(OCCN(C)C)(C5)C4)c2C)c(C(=O)O)n1] |
| D8a- L62 | 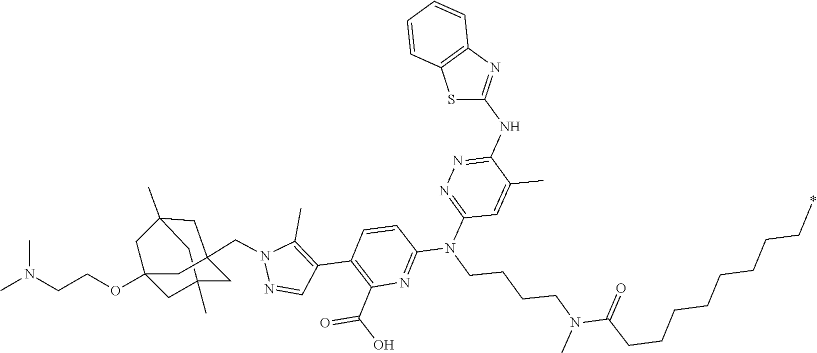 [SMILES: CCCCCCCCCC(=O)N(C)CCCCN(c1cc(C)c(Nc2nc3ccccc3s2)nn1)c1ccc(-c2cnn(CC34CC5(C)CC(C)(C3)CC(OCCN(C)C)(C5)C4)c2C)c(C(=O)O)n1] |
| D8a- L63 | 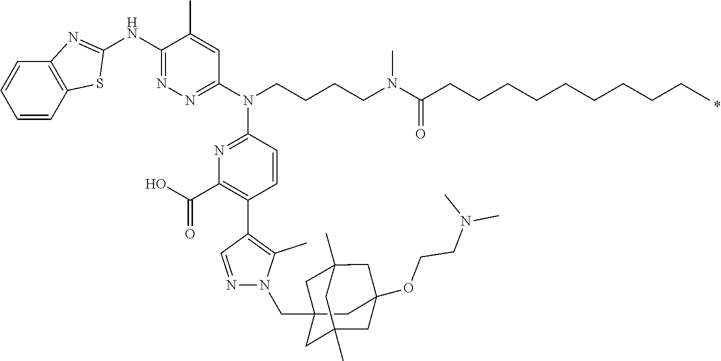 [SMILES: CCCCCCCCCCC(=O)N(C)CCCCN(c1cc(C)c(Nc2nc3ccccc3s2)nn1)c1ccc(-c2cnn(CC34CC5(C)CC(C)(C3)CC(OCCN(C)C)(C5)C4)c2C)c(C(=O)O)n1] |
| D8a- L64 | 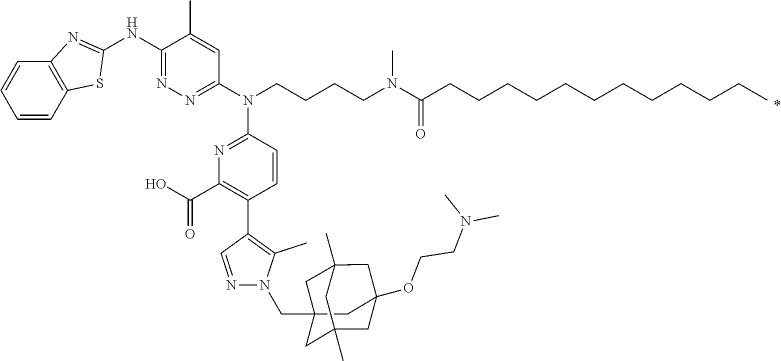 [SMILES: CCCCCCCCCCCCC(=O)N(C)CCCCN(c1cc(C)c(Nc2nc3ccccc3s2)nn1)c1ccc(-c2cnn(CC34CC5(C)CC(C)(C3)CC(OCCN(C)C)(C5)C4)c2C)c(C(=O)O)n1] |
| D2a- L65 | 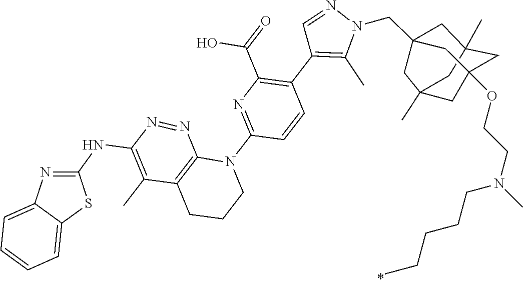 [SMILES: CCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D2a- L66 | 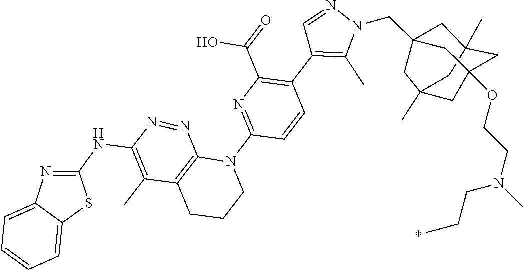 [SMILES: CCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D2a- L67 | 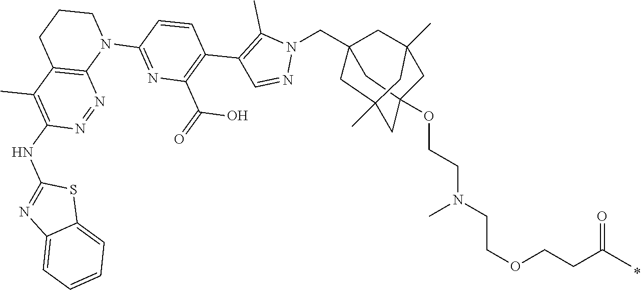 [SMILES: CC(=O)CCOCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D2a- L68 | 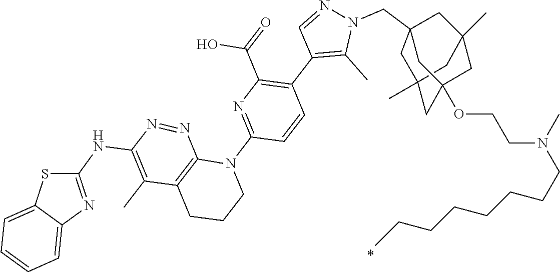 [SMILES: CCCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D2a- L69 | 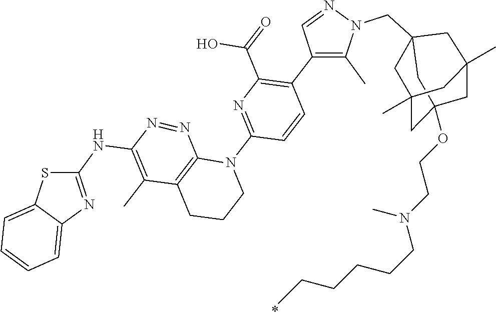 [SMILES: CCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D2a- L70 | 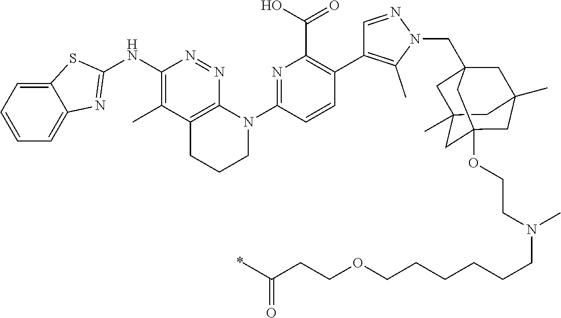 [SMILES: CC(=O)CCOCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D2a- L71 | 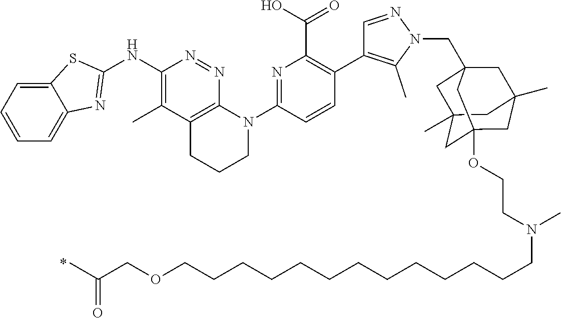 [SMILES: CC(=O)COCCCCCCCCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D8a- L35 | 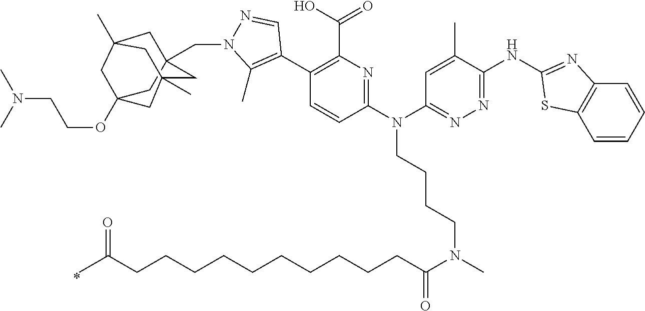 [SMILES: CC(=O)CCCCCCCCCCC(=O)N(C)CCCCN(c1cc(C)c(Nc2nc3ccccc3s2)nn1)c1ccc(-c2cnn(CC34CC5(C)CC(C)(C3)CC(OCCN(C)C)(C5)C4)c2C)c(C(=O)O)n1] |
| D2a- L72 | 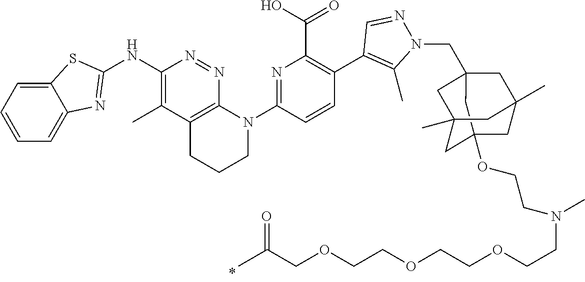 [SMILES: CC(=O)COCCOCCOCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D8a- L73 | 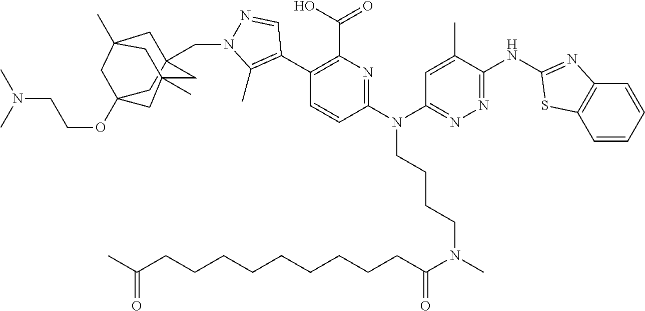 [SMILES: CC(=O)CCCCCCCCCC(=O)N(C)CCCCN(c1cc(C)c(Nc2nc3ccccc3s2)nn1)c1ccc(-c2cnn(CC34CC5(C)CC(C)(C3)CC(OCCN(C)C)(C5)C4)c2C)c(C(=O)O)n1] |
| D8a- L74 | 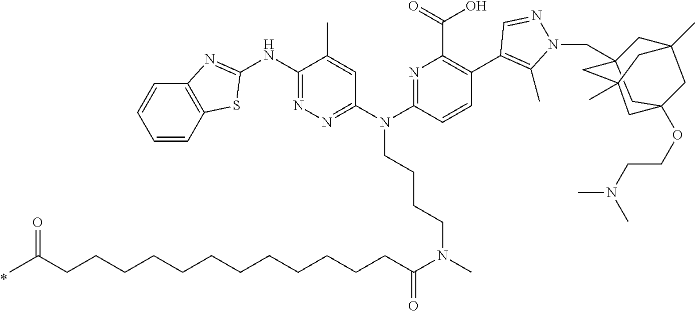 [SMILES: CC(=O)CCCCCCCCCCCCC(=O)N(C)CCCCN(c1cc(C)c(Nc2nc3ccccc3s2)nn1)c1ccc(-c2cnn(CC34CC5(C)CC(C)(C3)CC(OCCN(C)C)(C5)C4)c2C)c(C(=O)O)n1] |
| D8a- L75 | 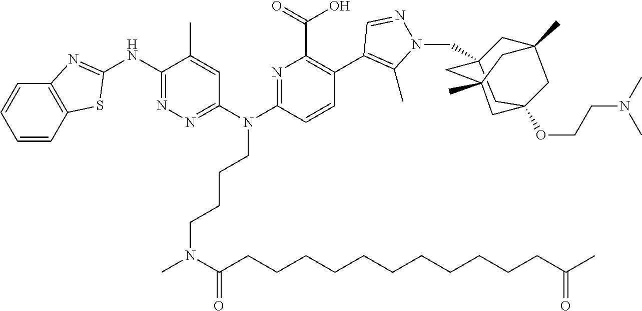 [SMILES: CC(=O)CCCCCCCCCCCC(=O)N(C)CCCCN(c1cc(C)c(Nc2nc3ccccc3s2)nn1)c1ccc(-c2cnn(C[C@]34C[C@]5(C)C[C@](C)(C3)C[C@@](OCCN(C)C)(C5)C4)c2C)c(C(=O)O)n1] |
| D9a- L76 | 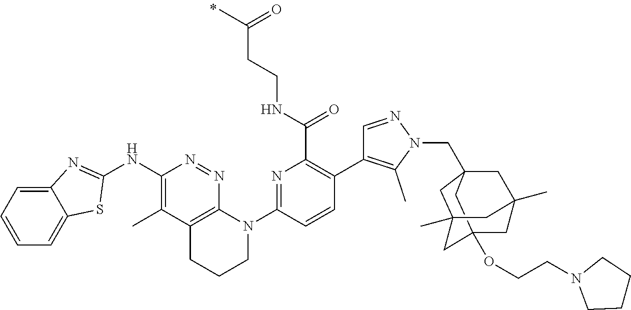 [SMILES: CC(=O)CCNC(=O)c1nc(N2CCCc3c2nnc(Nc2nc4ccccc4s2)c3C)ccc1-c1cnn(CC23CC4(C)CC(C)(C2)CC(OCCN2CCCC2)(C4)C3)c1C] |
| D8a- L77 | 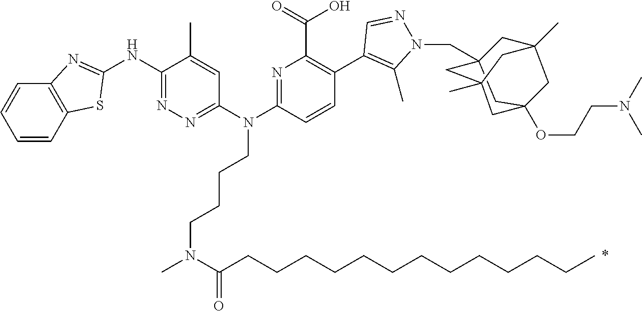 [SMILES: CCCCCCCCCCCCCC(=O)N(C)CCCCN(c1cc(C)c(Nc2nc3ccccc3s2)nn1)c1ccc(-c2cnn(CC34CC5(C)CC(C)(C3)CC(OCCN(C)C)(C5)C4)c2C)c(C(=O)O)n1] |
| D2a- L78 | 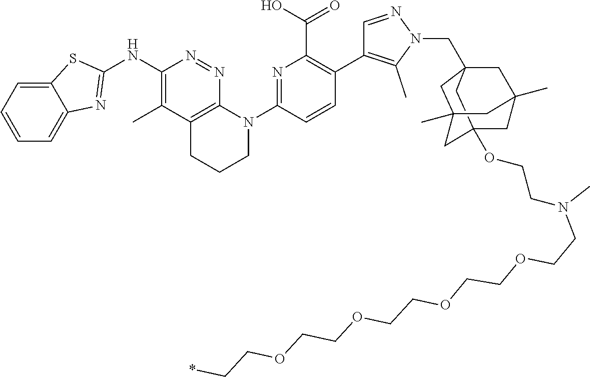 [SMILES: *CCOCCOCCOCCOCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D1a- L51 | 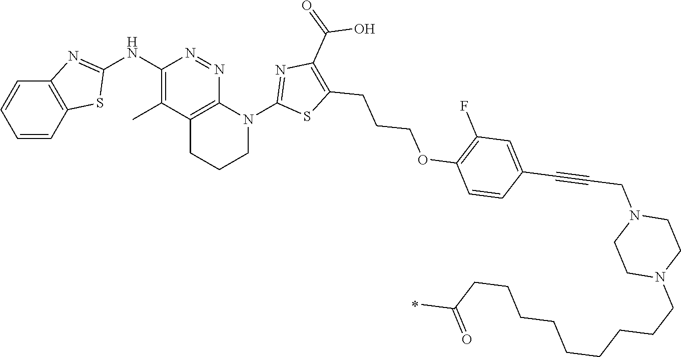 [SMILES: CC(=O)CCCCCCCCCN1CCN(CC#Cc2ccc(OCCCc3sc(N4CCCc5c4nnc(Nc4nc6ccccc6s4)c5C)nc3C(=O)O)c(F)c2)CC1] |
| D5a- L81 | 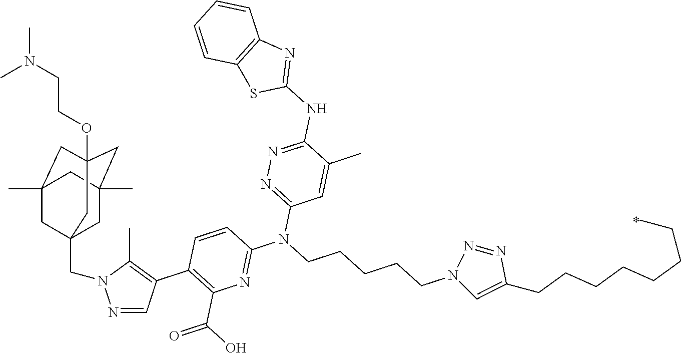 [SMILES: CCCCCCCCc1cn(CCCCCN(c2cc(C)c(Nc3nc4ccccc4s3)nn2)c2ccc(-c3cnn(CC45CC6(C)CC(C)(C4)CC(OCCN(C)C)(C6)C5)c3C)c(C(=O)O)n2)nn1] |
| D5a- L82 | 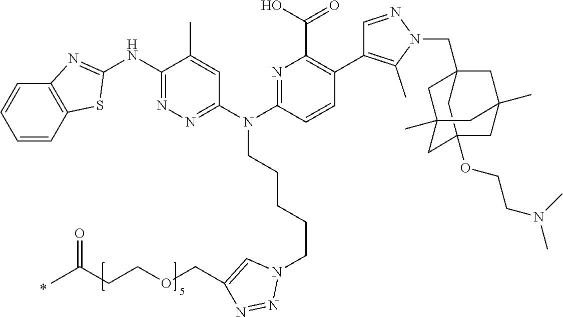 |
| D5a- L83 | 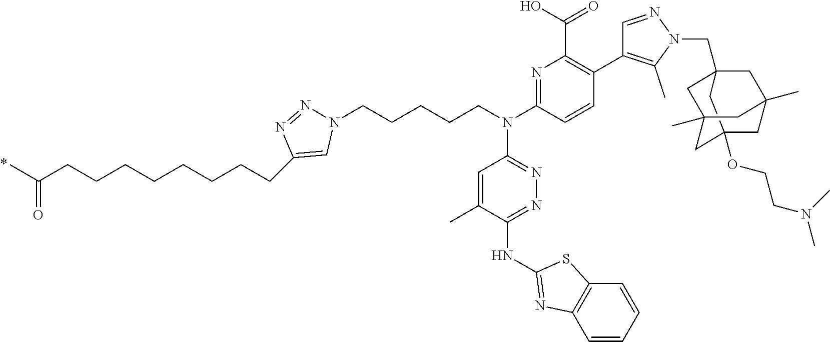 [SMILES: CC(=O)CCCCCCCCc1cn(CCCCCN(c2cc(C)c(Nc3nc4ccccc4s3)nn2)c2ccc(-c3cnn(CC45CC6(C)CC(C)(C4)CC(OCCN(C)C)(C6)C5)c3C)c(C(=O)O)n2)nn1] |
| D5a- L84 | 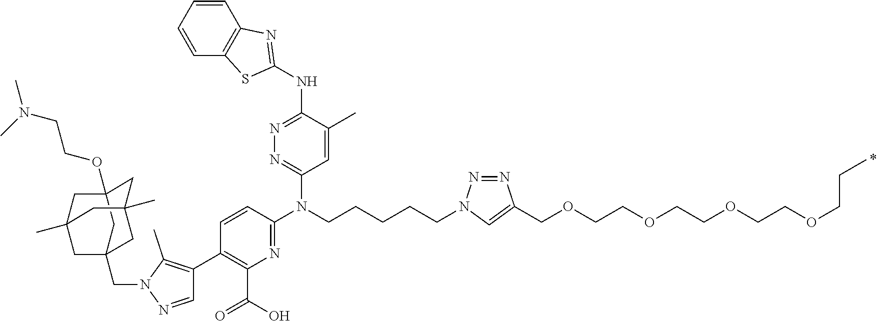 [SMILES: CCCOCCOCCOCCOCc1cn(CCCCCN(c2cc(C)c(Nc3nc4ccccc4s3)nn2)c2ccc(-c3cnn(CC45CC6(C)CC(C)(C4)CC(OCCN(C)C)(C6)C5)c3C)c(C(=O)O)n2)nn1] |
| D13a- L6 | 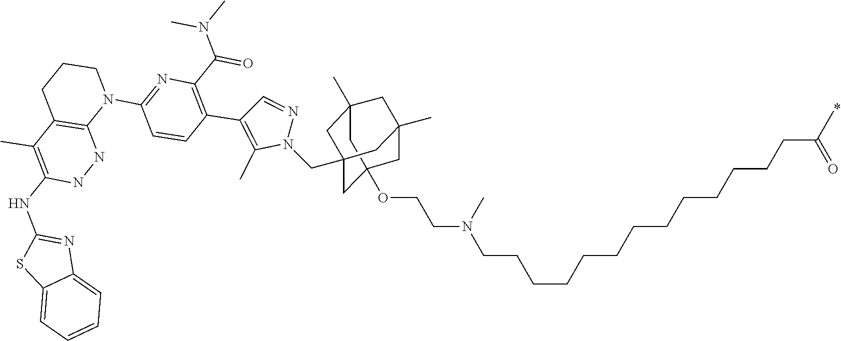 [SMILES: CC(=O)CCCCCCCCCCCCCN(C)CCOC12CC3(C)CC(C)(CC(Cn4ncc(-c5ccc(N6CCCC7=C6NNC(Nc6nc8ccccc8s6)=C7C)nc5C(=O)N(C)C)c4C)(C3)C1)C2] |
| D14a- L6 | 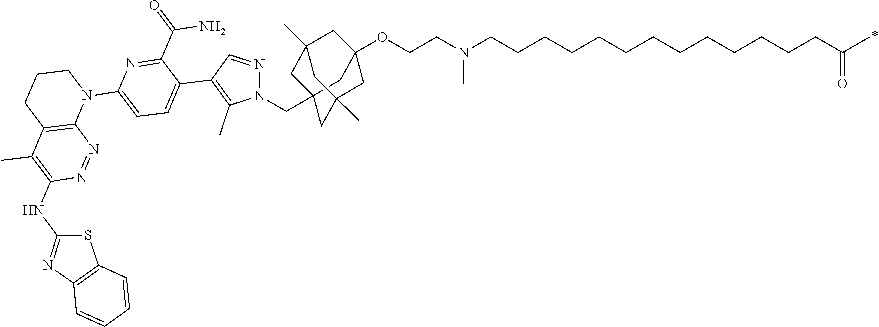 [SMILES: CC(=O)CCCCCCCCCCCCCN(C)CCOC12CC3(C)CC(C)(CC(Cn4ncc(-c5ccc(N6CCCc7c6nnc(Nc6nc8ccccc8s6)c7C)nc5C(N)=O)c4C)(C3)C1)C2] |
| D9a- L86 | 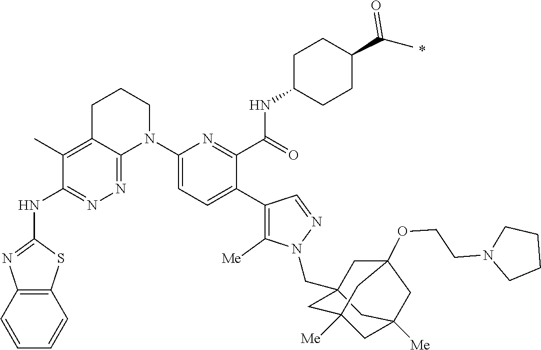 [SMILES: Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(C(=O)N[C@H]2CC[C@H](C(C)=O)CC2)n1] |
| D9a- L88 | 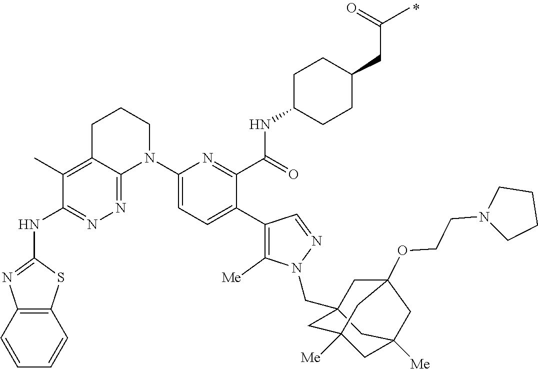 [SMILES: CC(=O)C[C@H]1CC[C@H](NC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)CC1] |
| D9a- L89 | 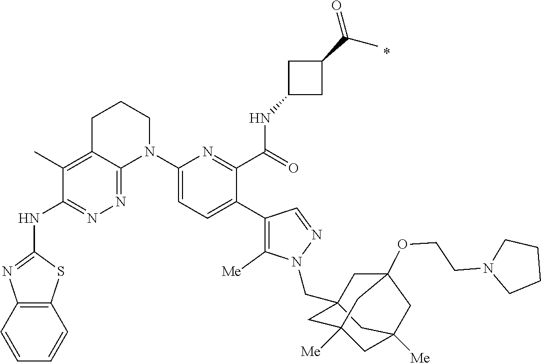 [SMILES: Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(C(=O)N[C@H]2C[C@H](C(C)=O)C2)n1] |
| D9a- L95 | 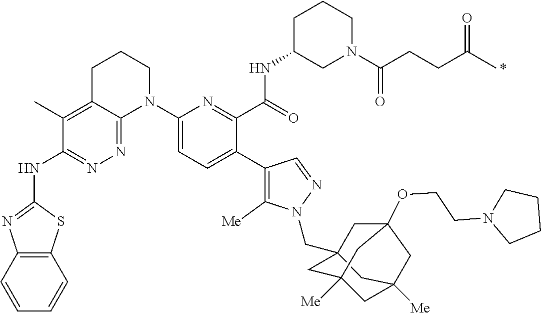 [SMILES: CC(=O)CCC(=O)N1CCC[C@@H](NC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C1] |
| D2a- L96 | 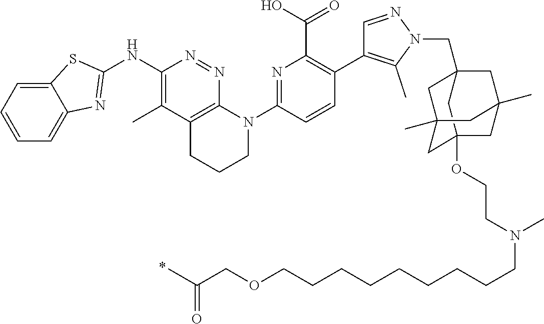 [SMILES: CC(=O)COCCCCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D2a- L85 | 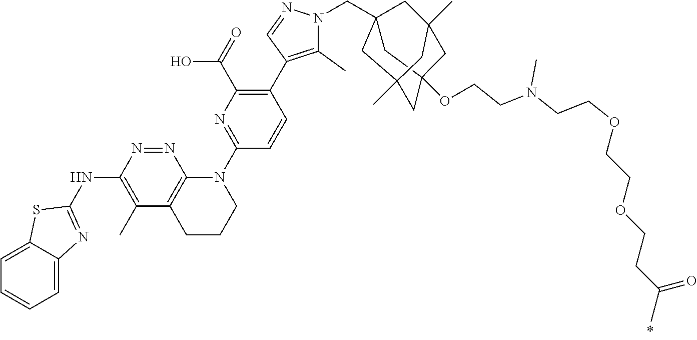 [SMILES: CC(=O)CCOCCOCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D1a- L16 | 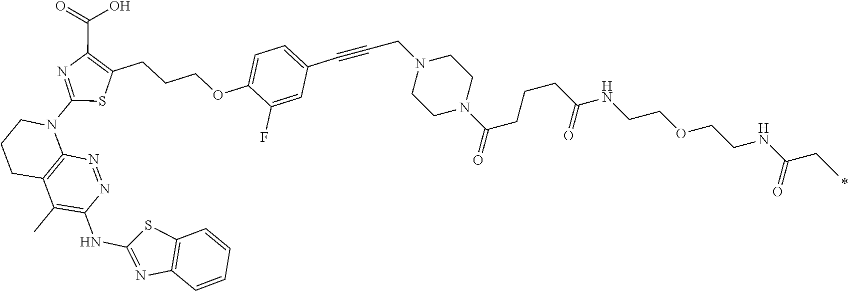 [SMILES: CCC(=O)NCCOCCNC(=O)CCCC(=O)N1CCN(CC#Cc2ccc(OCCCc3sc(N4CCCc5c4nnc(Nc4nc6ccccc6s4)c5C)nc3C(=O)O)c(F)c2)CC1] |
| D2a- L97 | 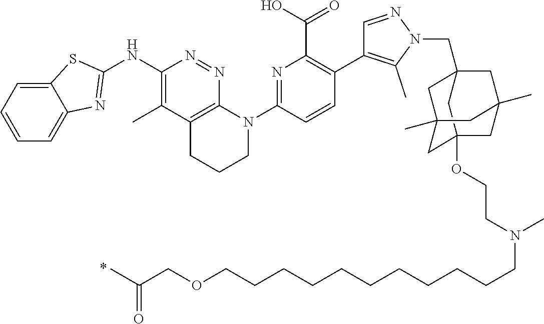 [SMILES: CC(=O)COCCCCCCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D9a- L99 | 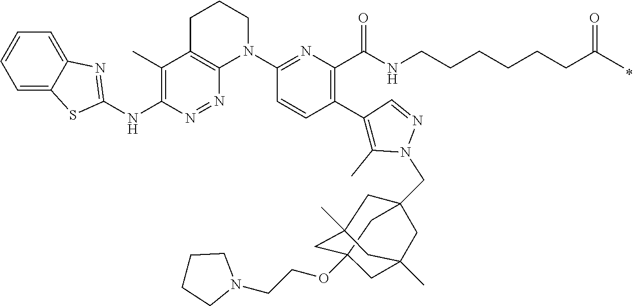 [SMILES: CC(=O)CCCCCCNC(=O)c1nc(N2CCCc3c2nnc(Nc2nc4ccccc4s2)c3C)ccc1-c1cnn(CC23CC4(C)CC(C)(C2)CC(OCCN2CCCC2)(C4)C3)c1C] |
| D9a- L100 | 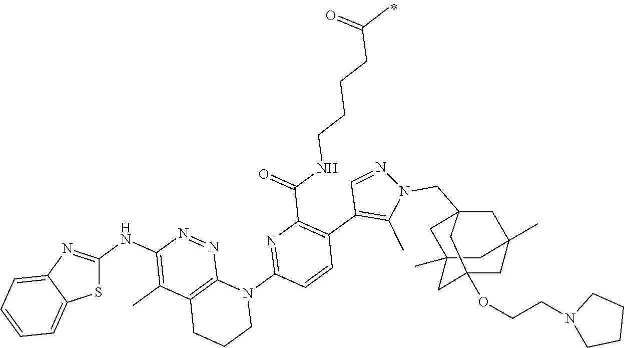 [SMILES: CC(=O)CCCCNC(=O)c1nc(N2CCCc3c2nnc(Nc2nc4ccccc4s2)c3C)ccc1-c1cnn(CC23CC4(C)CC(C)(C2)CC(OCCN2CCCC2)(C4)C3)c1C] |
| D9a- L101 | 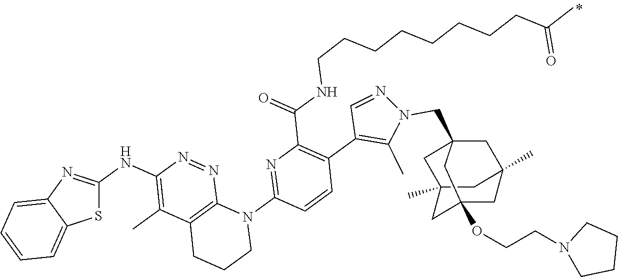 [SMILES: CC(=O)CCCCCCCCNC(=O)c1nc(N2CCCc3c2nnc(Nc2nc4ccccc4s2)c3C)ccc1-c1cnn(C[C@]23C[C@]4(C)C[C@](C)(C2)C[C@@](OCCN2CCCC2)(C4)C3)c1C] |
| D9a- L102 | 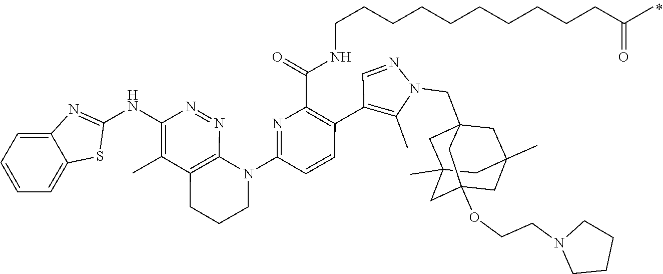 [SMILES: CC(=O)CCCCCCCCCCNC(=O)c1nc(N2CCCc3c2nnc(Nc2nc4ccccc4s2)c3C)ccc1-c1cnn(CC23CC4(C)CC(C)(C2)CC(OCCN2CCCC2)(C4)C3)c1C] |
| D12a- L35 | 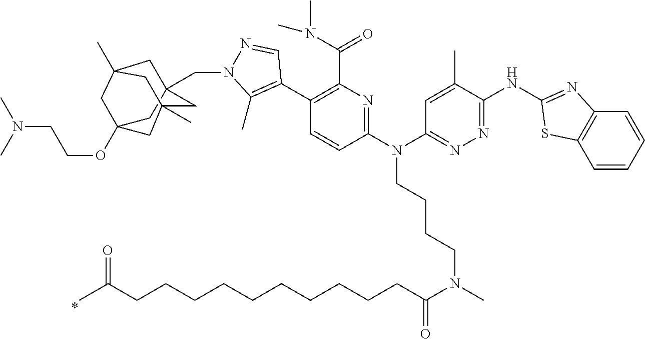 [SMILES: CC(=O)CCCCCCCCCCC(=O)N(C)CCCCN(c1cc(C)c(Nc2nc3ccccc3s2)nn1)c1ccc(-c2cnn(CC34CC5(C)CC(C)(C3)CC(OCCN(C)C)(C5)C4)c2C)c(C(=O)N(C)C)n1] |
| D9a- L103 | 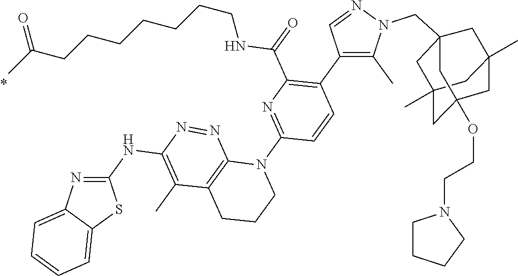 [SMILES: CC(=O)CCCCCCCNC(=O)c1nc(N2CCCc3c2nnc(Nc2nc4ccccc4s2)c3C)ccc1-c1cnn(CC23CC4(C)CC(C)(C2)CC(OCCN2CCCC2)(C4)C3)c1C] |
| D9a- L104 | 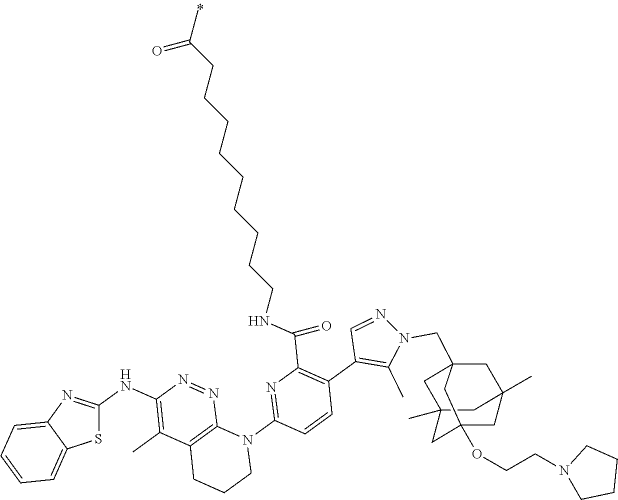 [SMILES: CC(=O)CCCCCCCCCNC(=O)c1nc(N2CCCc3c2nnc(Nc2nc4ccccc4s2)c3C)ccc1-c1cnn(CC23CC4(C)CC(C)(C2)CC(OCCN2CCCC2)(C4)C3)c1C] |
| D9a- L105 | 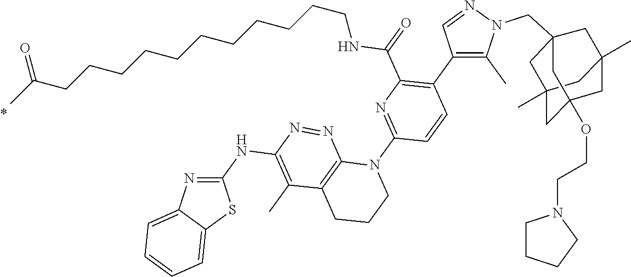 [SMILES: CC(=O)CCCCCCCCCCCNC(=O)c1nc(N2CCCc3c2nnc(Nc2nc4ccccc4s2)c3C)ccc1-c1cnn(CC23CC4(C)CC(C)(C2)CC(OCCN2CCCC2)(C4)C3)c1C] |
| D2a- L98 | 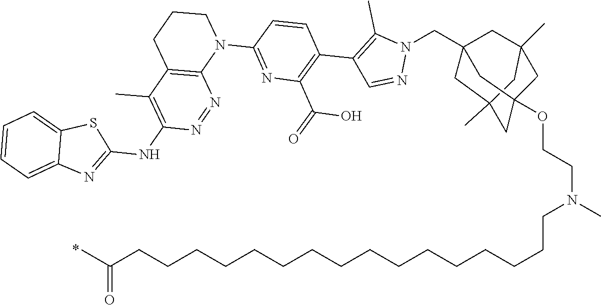 [SMILES: CC(=O)CCCCCCCCCCCCCCCCN(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| D10a- L80 | 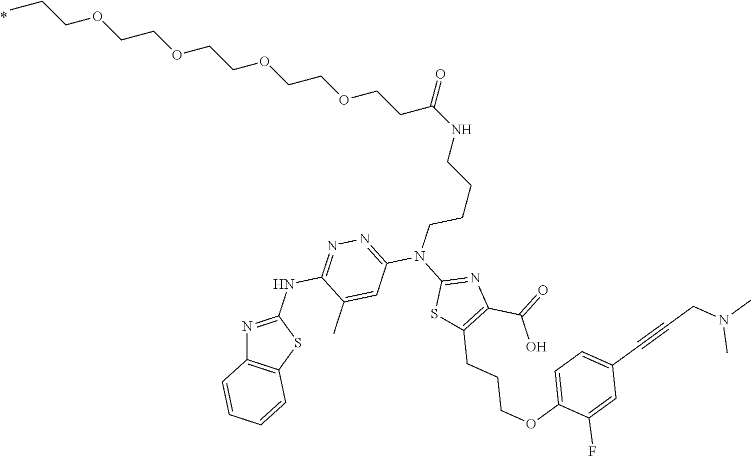 [SMILES: CCCOCCOCCOCCOCCC(=O)NCCCCN(c1cc(C)c(Nc2nc3ccccc3s2)nn1)c1nc(C(=O)O)c(CCCOc2ccc(C#CCN(C)C)cc2F)s1] |
| D15a- L6 | 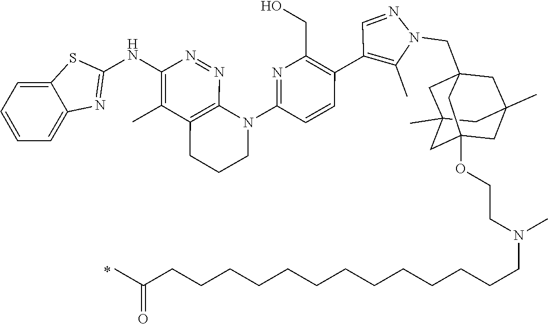 [SMILES: CC(=O)CCCCCCCCCCCCCN(C)CCOC12CC3(C)CC(C)(CC(Cn4ncc(-c5ccc(N6CCCc7c6nnc(Nc6nc8ccccc8s6)c7C)nc5CO)c4C)(C3)C1)C2] |
| D16a- L6 | 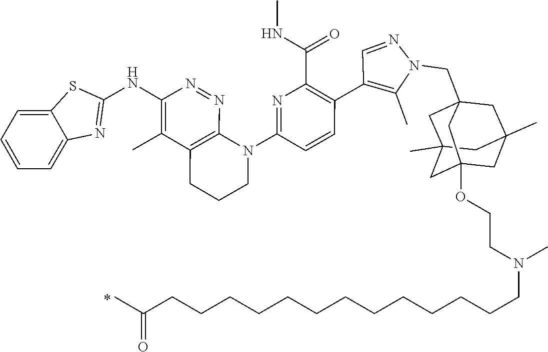 [SMILES: CNC(=O)c1nc(N2CCCc3c2nnc(Nc2nc4ccccc4s2)c3C)ccc1-c1cnn(CC23CC4(C)CC(C)(C2)CC(OCCN(C)CCCCCCCCCCCCCC(C)=O)(C4)C3)c1C] |
| D17a- L6 | 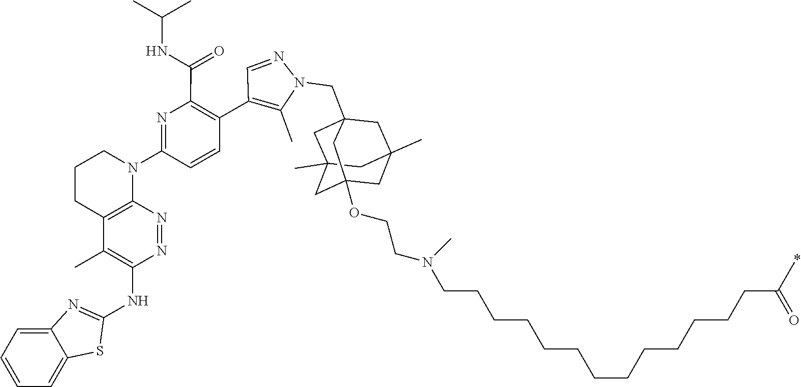 [SMILES: CC(=O)CCCCCCCCCCCCCN(C)CCOC12CC3(C)CC(C)(CC(Cn4ncc(-c5ccc(N6CCCc7c6nnc(Nc6nc8ccccc8s6)c7C)nc5C(=O)NC(C)C)c4C)(C3)C1)C2] |
| D9a- L106 | 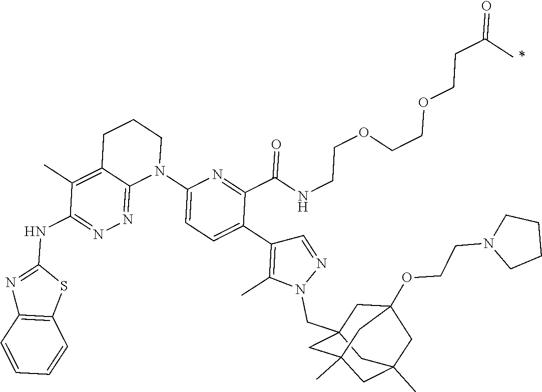 [SMILES: CC(=O)CCOCCOCCNC(=O)c1nc(N2CCCc3c2nnc(Nc2nc4ccccc4s2)c3C)ccc1-c1cnn(CC23CC4(C)CC(C)(C2)CC(OCCN2CCCC2)(C4)C3)c1C] |
| D9a- L107 | 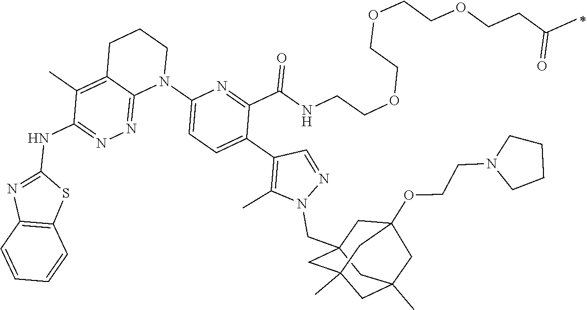 [SMILES: CC(=O)CCOCCOCCOCCNC(=O)c1nc(N2CCCc3c2nnc(Nc2nc4ccccc4s2)c3C)ccc1-c1cnn(CC23CC4(C)CC(C)(C2)CC(OCCN2CCCC2)(C4)C3)c1C] |
| D9a- L108 | 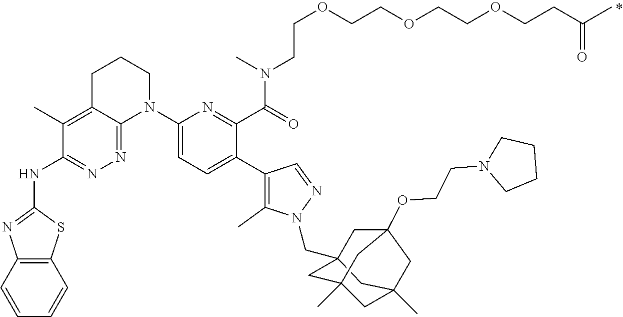 [SMILES: CC(=O)CCOCCOCCOCCN(C)C(=O)c1nc(N2CCCc3c2nnc(Nc2nc4ccccc4s2)c3C)ccc1-c1cnn(CC23CC4(C)CC(C)(C2)CC(OCCN2CCCC2)(C4)C3)c1C] |
| D9a- L109 | 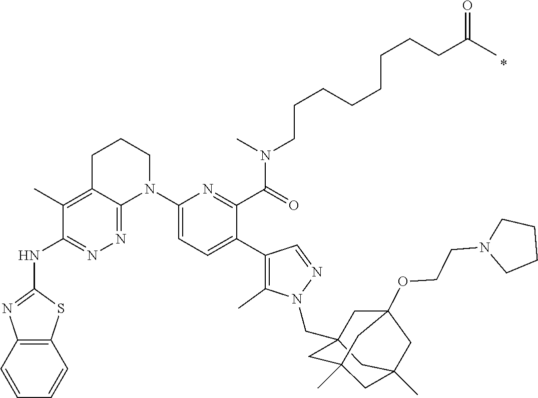 [SMILES: CC(=O)CCCCCCCCN(C)C(=O)c1nc(N2CCCc3c2nnc(Nc2nc4ccccc4s2)c3C)ccc1-c1cnn(CC23CC4(C)CC(C)(C2)CC(OCCN2CCCC2)(C4)C3)c1C] |
| D9a- L90 | 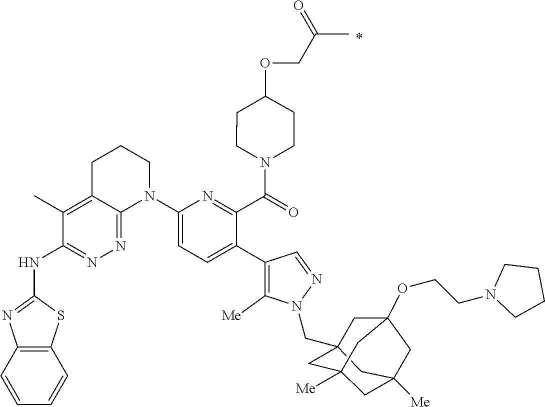 [SMILES: CC(=O)COC1CCN(C(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)CC1] |
| D9a- L91 | 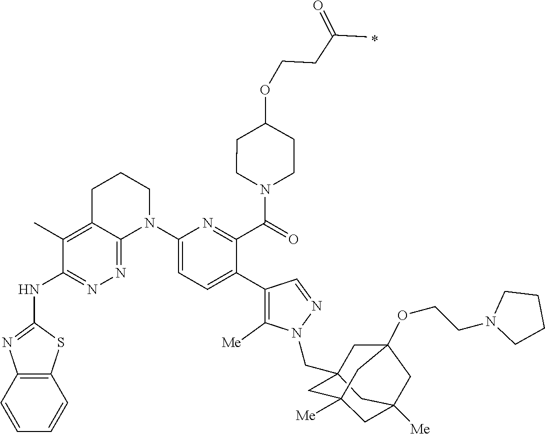 [SMILES: CC(=O)CCOC1CCN(C(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)CC1] |
| D9a- L92 | 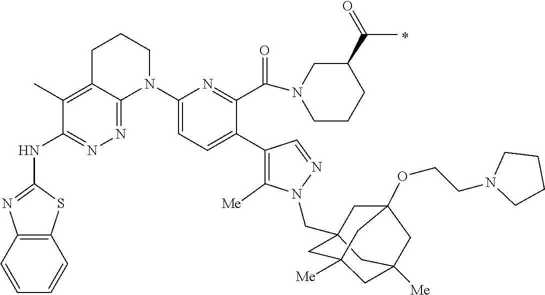 [SMILES: CC(=O)[C@H]1CCCN(C(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C1] |
| D9a- L93 | 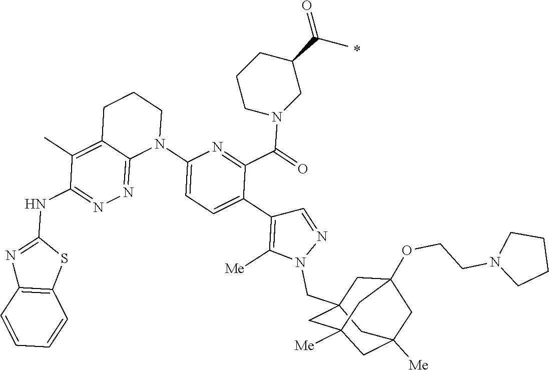 [SMILES: CC(=O)[C@@H]1CCCN(C(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C1] |
| D9a- L94 | 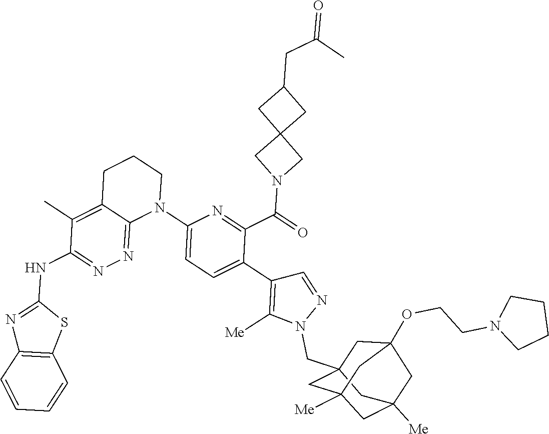 [SMILES: CC(=O)CC1CC2(C1)CN(C(=O)c1nc(N3CCCc4c3nnc(Nc3nc5ccccc5s3)c4C)ccc1-c1cnn(CC34CC5(C)CC(C)(C3)CC(OCCN3CCCC3)(C5)C4)c1C)C2] |
| D11a- L5 | 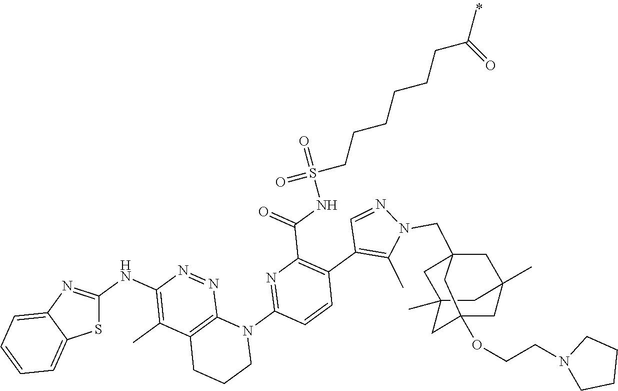 [SMILES: CC(=O)CCCCCCS(=O)(=O)NC(=O)c1nc(N2CCCc3c2nnc(Nc2nc4ccccc4s2)c3C)ccc1-c1cnn(CC23CC4(C)CC(C)(C2)CC(OCCN2CCCC2)(C4)C3)c1C] |
| D9a- L87 | 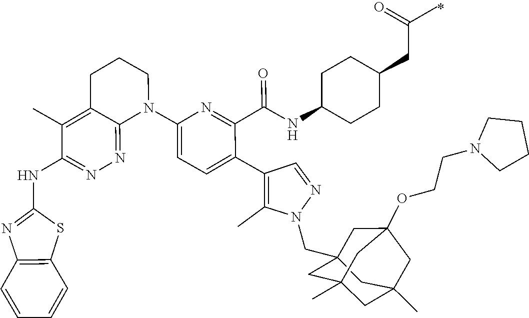 [SMILES: CC(=O)C[C@H]1CC[C@@H](NC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)CC1] |

3. Degradation Signaling Moieties

Degradation signaling compounds and moieties (DSMs) of the present disclosure include compounds and moieties thereof that induce degradation of the targeted Bcl proteins (e.g., Bcl-xL). DSMs degrade Bcl by binding or recruiting at least one degradation protein, which is usually associated with the proteasome, the ubiquitin-proteasome pathways, or lysosomal proteolysis. DSMs of this disclosure include, but are not limited to, E3 ubiquitin ligase recognition agents. In some embodiments, the E3 ubiquitin ligase or component of the E3 ubiquitin ligase complex targeted is MDM2, cIAPI, VHL protein, CBRN or SCF^{β-TRCP}.

The E3 ligase recognition agent is any compound that effectively binds to an E3 ubiquitin ligase or an E3 ubiquitin ligase complex. In some embodiments, the E3 ligase recognition agent is an E3 ubiquitin ligase ligand, such as a VHL ligand, a thalidomide cereblon binder, or an inhibitor of apoptosis (IAP) E3 ligases. As used herein, a “thalidomide cereblon binder” refers to thalidomide or thalidomide derivatives (e.g., pomalidomide or a modified version of pomalidomide) that binds to cereblon. Exemplary E3 ligase recognition agents are those described in WO 2021/007307, WO 2020/163823, US 2019/0127359, WO 2019/144117, WO 2018/200981, WO 2016/149668, WO 2016/105518, WO 2017/184995, WO 2017/007612, WO 2015/160845, Girardini, M. et al., “Cereblon versus VHL: Hijacking E3 ligases against each other using PROTACs”, Bioorganic & Medicinal Chemistry 27 (2019) 2466-79, Zhang, X. et al., “Discovery of IAP-recruiting BDL-X_{L} PROTACs as potent degraders across multiple cancern cell lines”, European Journal of Medicinal Chemistry 199 (2020) 112397, and Chang, Yung-Chieh et al., “An Updated Review of Smac Mimetics, LCL161, Birinapant, and GDC-0162 in Cancer Treatment”, Applied Sciences, 2021, 11, 335, each of which are incorporated herein by reference.

In some embodiments, DSM represents a degradation signaling compound attached to the linker by a covalent bond, where a degradation signaling compound (DSM compound) is selected from a compound in Table 5, or an enantiomer, a diastereomer, and/or a pharmaceutically acceptable salt thereof.

TABLE 5
Exemplary Degradation Signaling Compounds

| | |
|---|---|
| DSM1 | 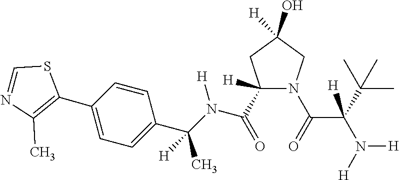 [SMILES: Cc1ncsc1-c1ccc([C@H](C)NC(=O)[C@@H]2C[C@@H](O)CN2C(=O)[C@@H](N)C(C)(C)C)cc1] |
| DSM2 | 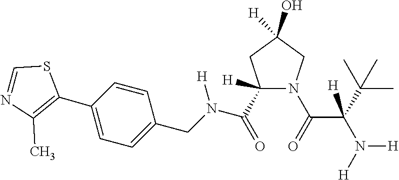 [SMILES: Cc1ncsc1-c1ccc(CNC(=O)[C@@H]2C[C@@H](O)CN2C(=O)[C@@H](N)C(C)(C)C)cc1] |
| DSM3 | 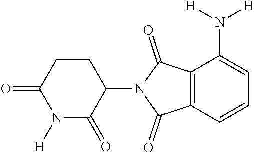 [SMILES: Nc1cccc2c1C(=O)N(C1CCC(=O)NC1=O)C2=O] |
| DSM4 | 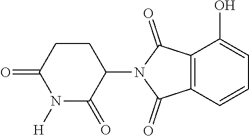 [SMILES: O=C1CCC(N2C(=O)c3cccc(O)c3C2=O)C(=O)N1] |
| DSM5 | 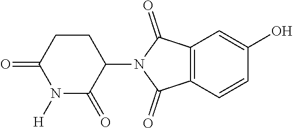 [SMILES: O=C1CCC(N2C(=O)c3ccc(O)cc3C2=O)C(=O)N1] |
| DSM6 | 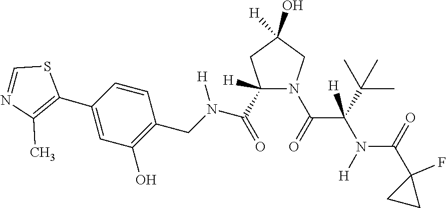 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1] |
| DSM7 | 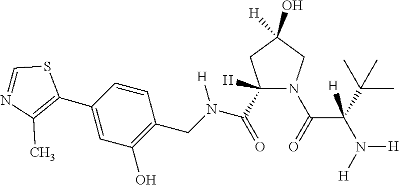 [SMILES: Cc1ncsc1-c1ccc(CNC(=O)[C@@H]2C[C@@H](O)CN2C(=O)[C@@H](N)C(C)(C)C)c(O)c1] |
| DSM8 | 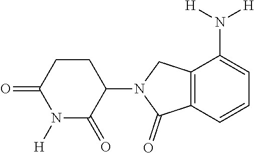 [SMILES: Nc1cccc2c1CN(C1CCC(=O)NC1=O)C2=O] |
| DSM9 | 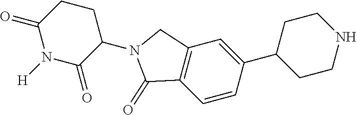 [SMILES: O=C1CCC(N2Cc3cc(C4CCNCC4)ccc3C2=O)C(=O)N1] |
| DSM10 | 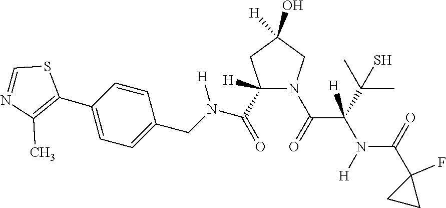 [SMILES: Cc1ncsc1-c1ccc(CNC(=O)[C@@H]2C[C@@H](O)CN2C(=O)[C@@H](NC(=O)C2(F)CC2)C(C)(C)S)cc1] |
| DSM11 | 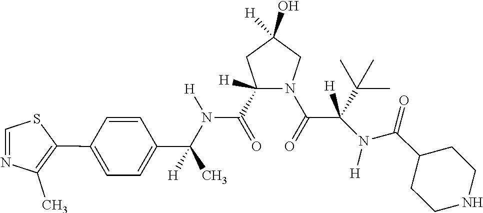 [SMILES: Cc1ncsc1-c1ccc([C@H](C)NC(=O)[C@@H]2C[C@@H](O)CN2C(=O)[C@@H](NC(=O)C2CCNCC2)C(C)(C)C)cc1] |
| DSM12 | 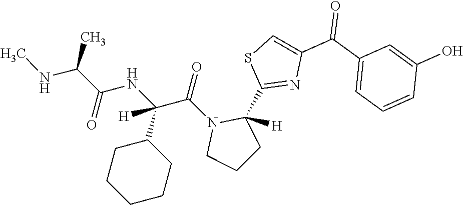 [SMILES: CN[C@@H](C)C(=O)N[C@H](C(=O)N1CCC[C@H]1c1nc(C(=O)c2cccc(O)c2)cs1)C1CCCCC1] |

In some embodiments, DSM in Formula (A) is represented by a formula in Table 6, or an enantiomer, a diastereomer and/or a pharmaceutically acceptable salt thereof, where -* represents a bond to the linker (L).

TABLE 6
Exemplary Degradation Signaling Compounds Showing Point of
Attachment to the Linker (L)

| | |
|---|---|
| DSM1a | 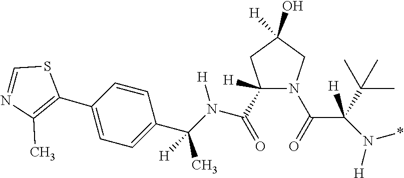 [SMILES: *N[C@H](C(=O)N1C[C@H](O)C[C@H]1C(=O)N[C@@H](C)c1ccc(-c2scnc2C)cc1)C(C)(C)C] |
| DSM2a | 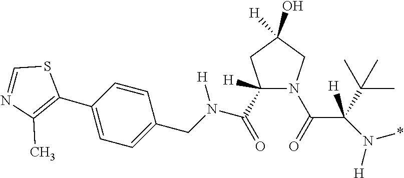 [SMILES: *N[C@H](C(=O)N1C[C@H](O)C[C@H]1C(=O)NCc1ccc(-c2scnc2C)cc1)C(C)(C)C] |
| DSM3a | 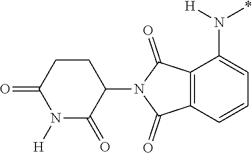 [SMILES: *Nc1cccc2c1C(=O)N(C1CCC(=O)NC1=O)C2=O] |
| DSM4a | 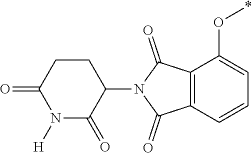 [SMILES: *Oc1cccc2c1C(=O)N(C1CCC(=O)NC1=O)C2=O] |
| DSM5a | 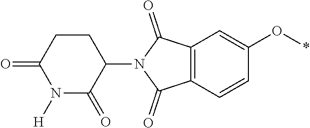 [SMILES: *Oc1ccc2c(c1)C(=O)N(C1CCC(=O)NC1=O)C2=O] |
| DSM6a | 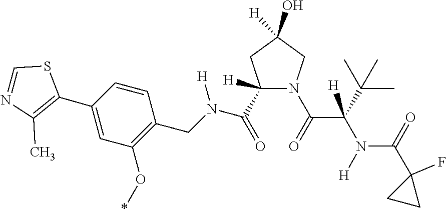 [SMILES: *Oc1cc(-c2scnc2C)ccc1CNC(=O)[C@@H]1C[C@@H](O)CN1C(=O)[C@@H](NC(=O)C1(F)CC1)C(C)(C)C] |
| DSM7a | 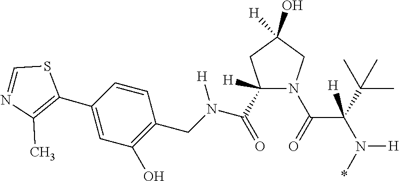 [SMILES: *N[C@H](C(=O)N1C[C@H](O)C[C@H]1C(=O)NCc1ccc(-c2scnc2C)cc1O)C(C)(C)C] |
| DSM8a | 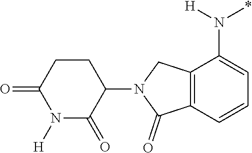 [SMILES: *Nc1cccc2c1CN(C1CCC(=O)NC1=O)C2=O] |
| DSM9a | 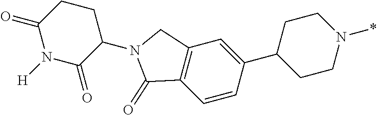 [SMILES: *N1CCC(c2ccc3c(c2)CN(C2CCC(=O)NC2=O)C3=O)CC1] |
| DSM10a | 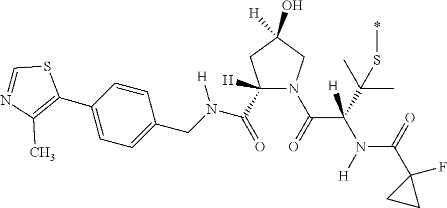 [SMILES: *SC(C)(C)[C@H](NC(=O)C1(F)CC1)C(=O)N1C[C@H](O)C[C@H]1C(=O)NCc1ccc(-c2scnc2C)cc1] |
| DSM11a | 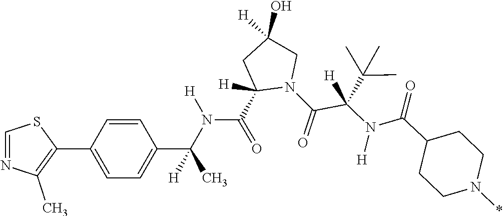 [SMILES: *N1CCC(C(=O)N[C@H](C(=O)N2C[C@H](O)C[C@H]2C(=O)N[C@@H](C)c2ccc(-c3scnc3C)cc2)C(C)(C)C)CC1] |
| DSM12a | 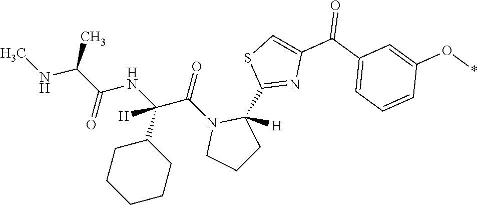 [SMILES: *Oc1cccc(C(=O)c2csc([C@@H]3CCCN3C(=O)[C@@H](NC(=O)[C@H](C)NC)C3CCCCC3)n2)c1] |

In some embodiments, DSM is DSM1a, or an enantiomer, a diastereomer, and/or a pharmaceutically acceptable salt thereof, where -* represents a bond to the linker (L):

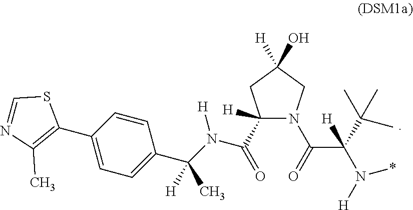 [SMILES: *N[C@H](C(=O)N1C[C@H](O)C[C@H]1C(=O)N[C@@H](C)c1ccc(-c2scnc2C)cc1)C(C)(C)C]

4. Bifunctional Bcl-xL Degrader Compounds

In some embodiments, the Bcl-xL degrader compound is represented by Formula (A) described above (e.g., a compound described in any one of the first to twenty-eighth embodiments). In some embodiments, the Bcl-xL degrader compound is a compound in Table 7 or an enantiomer, a diastereoisomer and/or a pharmaceutically acceptable salt thereof.

Table 7
Exemplary Bifunctional Bcl-xL Degrader Compounds

| Ex. | |
|---|---|
| No | Compound Structure and Chemical Names |
| 1 | 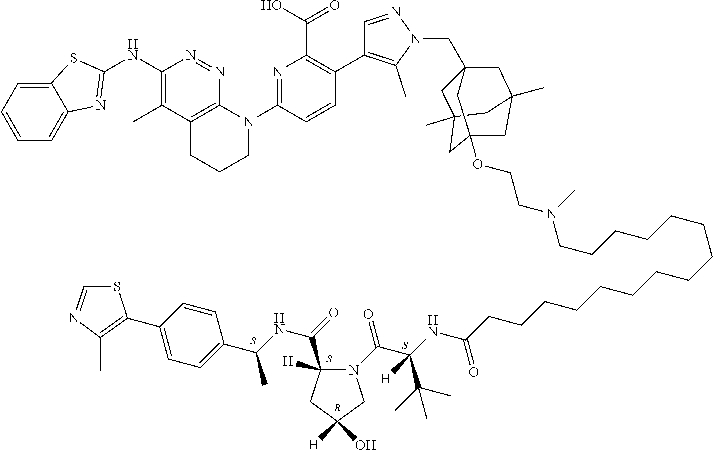 |
| | 6-[3-(1,3-benzothiazol-2-ylamino)-4-methyl-6,7-dihydro-5H-pyrido[2,3- |
| | c]pyridazin-8-yl]-3-[1-[[3-[2-[[16-[[(1S)-1-[(2S,4R)-4-hydroxy-2-[[(1S)-1-[4- |
| | (4-methyl thiazol-5-yl)phenyl]ethyl]carbamoyl]pyrrolidine-1-carbonyl]-2,2- |
| | dimethyl-propyl]amino]-16-oxo-hexadecyl]-methyl-amino]ethoxy]-5,7- |
| | dimethyl-1-adamantyl]methyl]-5-methyl-pyrazol-4-yl]pyridine-2-carboxylic |
| | acid |
| 2 | 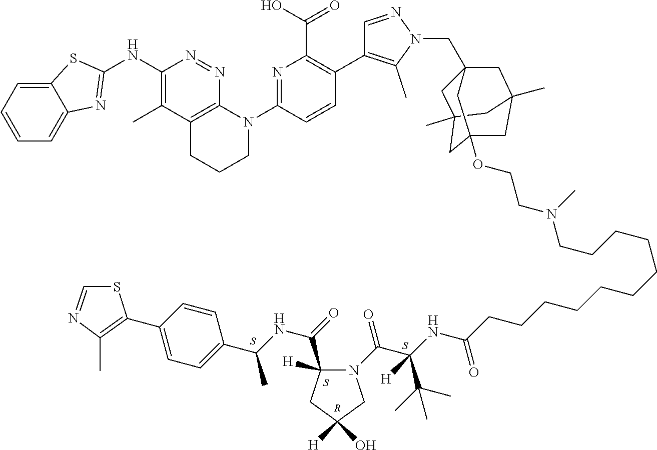 |
| | 6-[3-(1,3-benzothiazol-2-ylamino)-4-methyl-6,7-dihydro-5H-pyrido[2,3-c] |
| | pyridazin-8-yl]-3-[1-[[3-[2-[[12-[[(1S)-1-[(2S,4R)-4-hydroxy-2-[[(1S)-1-[4-(4- |
| | methylthiazol-5-yl)phenyl]ethyl]carbamoyl]pyrrolidine-1-carbonyl]-2,2- |
| | dimethyl-propyl]amino]-12-oxo-dodecyl]-methyl-amino]ethoxy]-5,7- |
| | dimethyl-1-adamantyl]methyl]-5-methyl-pyrazol-4-yl]pyridine-2-carboxylic |
| | acid |
| 3 | 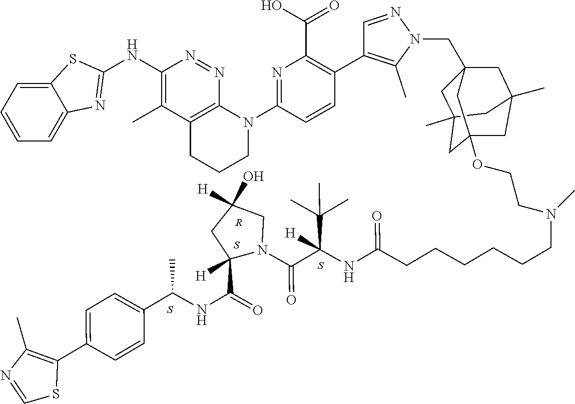 |
| | 6-[3-(1,3-benzothiazol-2-ylamino)-4-methyl-6,7-dihydro-5H-pyrido[2,3-c] |
| | pyridazin-8-yl]-3-[1-[[3-[2-[[7-[[(1S)-1-[(2S,4R)-4-hydroxy-2-[[(1S)-1-[4-(4- |
| | methylthiazol-5-yl)phenyl]ethyl]carbamoyl]pyrrolidine-1-carbonyl]-2,2- |
| | dimethyl-propyl]amino]-7-oxo-heptyl]-methyl-amino]ethoxy]-5,7-dimethyl-1- |
| | adamantyl]methyl]-5-methyl-pyrazol-4-yl]pyridine-2-carboxylic acid |
| 4 | 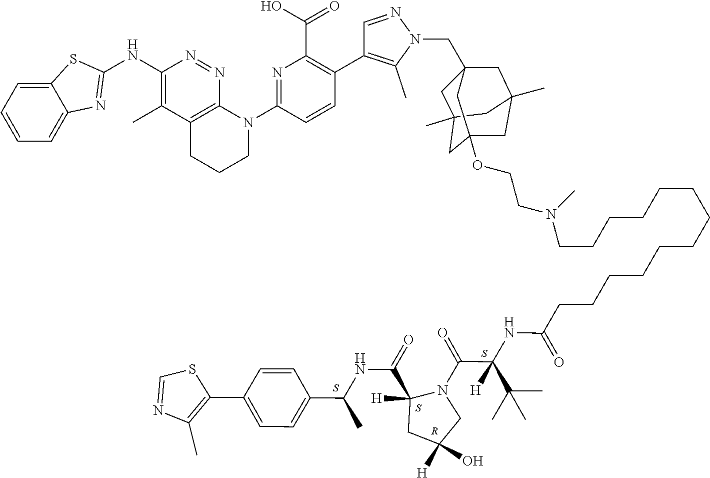 |
| | 6-[3-(1,3-benzothiazol-2-ylamino)-4-methyl-6,7-dihydro-5H-pyrido[2,3-c] |
| | pyridazin-8-yl]-3-[1-[[3-[2-[[14-[[(1S)-1-[(2S,4R)-4-hydroxy-2-[[(1S)-1-[4-(4- |
| | methylthiazol-5-yl)phenyl]ethyl]carbamoyl]pyrrolidine-1-carbonyl]-2,2- |
| | dimethyl-propyl]amino]-14-oxo-tetradecyl]-methyl-amino]ethoxy]-5,7- |
| | dimethyl-1-adamantyl]methyl]-5-methyl-pyrazol-4-yl]pyridine-2-carboxylic |
| | acid |
| 5 | 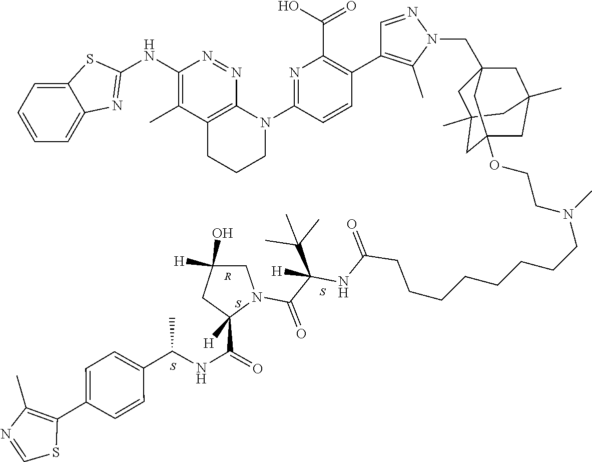 |
| | 6-[3-(1,3-benzothiazol-2-ylamino)-4-methyl-6,7-dihydro-5H-pyrido[2,3-c] |
| | pyridazin-8-yl]-3-[1-[[3-[2-[[9-[[(1S)-1-[(2S,4R)-4-hydroxy-2-[[(1S)-1-[4-(4- |
| | methylthiazol-5-yl)phenyl]ethyl]carbamoyl]pyrrolidine-1-carbonyl]-2,2- |
| | dimethyl-propyl]amino]-9-oxo-nonyl]-methyl-amino]ethoxy]-5,7-dimethyl-1- |
| | adamantyl]methyl]-5-methyl-pyrazol-4-yl]pyridine-2-carboxylic acid |
| 6 | 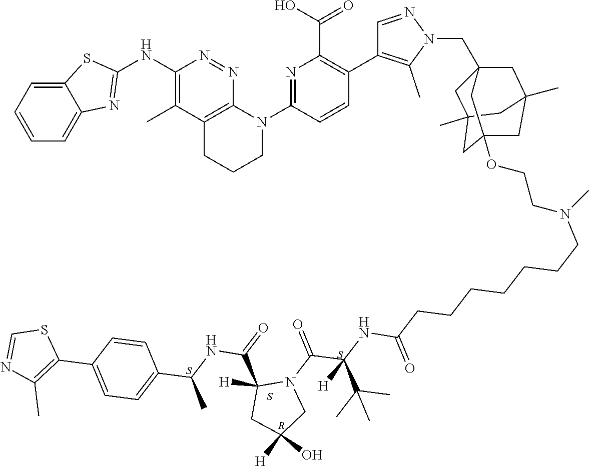 |
| | 6-[3-(1,3-benzothiazol-2-ylamino)-4-methyl-6,7-dihydro-5H-pyrido[2,3- |
| | c]pyridazin-8-yl]-3-[1-[[3-[2-[[8-[[(1S)-1-[(2S,4R)-4-hydroxy-2-[[(1S)-1-[4-(4- |
| | methylthiazol-5-yl)phenyl]ethyl]carbamoyl]pyrrolidine-1-carbonyl]-2,2- |
| | dimethyl-propyl]amino]-8-oxo-octyl]-methyl-amino]ethoxy]-5,7-dimethyl-1- |
| | adamantyl]methyl]-5-methyl-pyrazol-4-yl]pyridine-2-carboxylic acid |
| 7 | 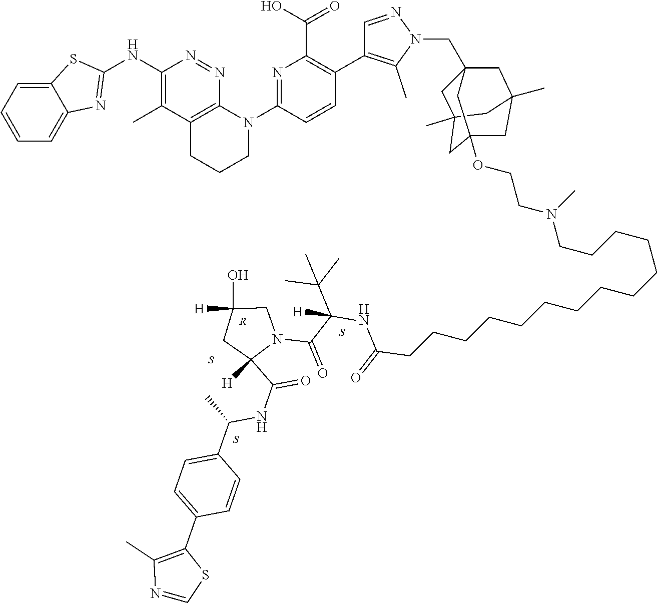 |
| | 6-[3-(1,3-benzothiazol-2-ylamino)-4-methyl-6,7-dihydro-5H-pyrido[2,3- |
| | c]pyridazin-8-yl]-3-[1-[[3-[2-[[15-[[(1S)-1-[(2S,4R)-4-hydroxy-2-[[(1S)-1-[4- |
| | (4-methylthiazol-5-yl)phenyl]ethyl]carbamoyl]pyrrolidine-1-carbonyl]-2,2- |
| | dimethyl-propyl]amino]-15-oxo-pentadecyl]-methyl-amino]ethoxy]-5,7- |
| | dimethyl-1-adamantyl]methyl]-5-methyl-pyrazol-4-yl]pyridine-2-carboxylic |
| | acid |
| 8 | 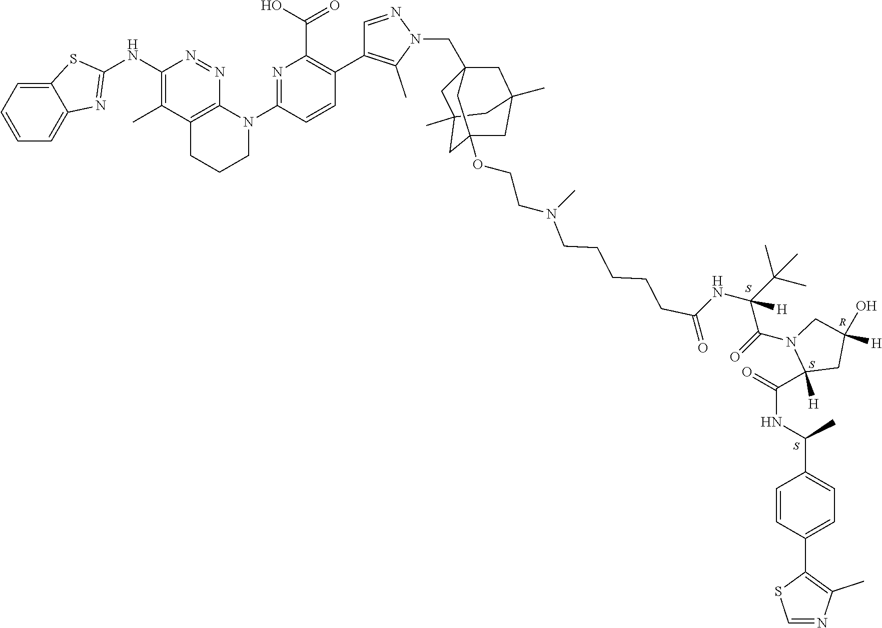 |
| | 6-[3-(1,3-benzothiazol-2-ylamino)-4-methyl-6,7-dihydro-5H-pyrido[2,3-c] |
| | pyridazin-8-yl]-3-[1-[[3-[2-[[6-[[(1S)-1-[(2S,4R)-4-hydroxy-2-[[(1S)-1-[4-(4- |
| | methylthiazol-5-yl)phenyl]ethyl]carbamoyl]pyrrolidine-1-carbonyl]-2,2- |
| | dimethyl-propyl]amino]-6-oxo-hexyl]-methyl-amino]ethoxy]-5,7-dimethyl-1- |
| | adamantyl]methyl]-5-methyl-pyrazol-4-yl]pyridine-2-carboxylic acid |
| 9 | 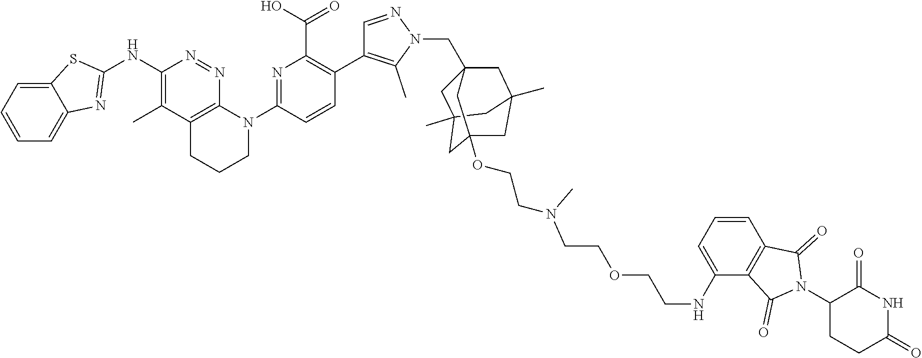 [SMILES: Cc1c(Nc2nc3ccccc3s2)nnc2c1CCCN2c1ccc(-c2cnn(CC34CC5(C)CC(C)(C3)CC(OCCN(C)CCOCCNc3cccc6c3C(=O)N(C3CCC(=O)NC3=O)C6=O)(C5)C4)c2C)c(C(=O)O)n1] |
| | 6-[3-(1,3-benzothiazol-2-ylamino)-4-methyl-6,7-dihydro-5H-pyrido[2,3-c] |
| | pyridazin-8-yl]-3-[1-[[3-[2-[2-[2-[[2-(2,6-dioxo-3-piperidyl)-1,3-dioxo- |
| | isoindolin-4-yl]amino]ethoxy]ethyl-methyl-amino]ethoxy]-5,7-dimethyl-1- |
| | adamantyl]methyl]-5-methyl-pyrazol-4-yl]pyridine-2-carboxylic acid |
| 10 | 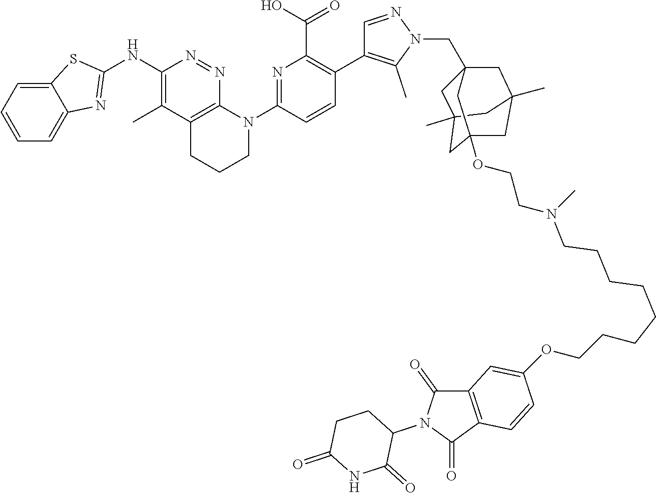 [SMILES: Cc1c(Nc2nc3ccccc3s2)nnc2c1CCCN2c1ccc(-c2cnn(CC34CC5(C)CC(C)(C3)CC(OCCN(C)CCCCCCCCOc3ccc6c(c3)C(=O)N(C3CCC(=O)NC3=O)C6=O)(C5)C4)c2C)c(C(=O)O)n1] |
| | 6-[3-(1,3-benzothiazol-2-ylamino)-4-methyl-6,7-dihydro-5H-pyrido[2,3-c] |
| | pyridazin-8-yl]-3-[1-[[3-[2-[8-[2-(2,6-dioxo-3-piperidyl)-1,3-dioxo-isoindolin- |
| | 5-yl]oxyoctyl-methyl-amino]ethoxy]-5,7-dimethyl-1-adamantyl]methyl]-5- |
| | methyl-pyrazol-4-yl]pyridine-2-carboxylic acid |
| 11 | 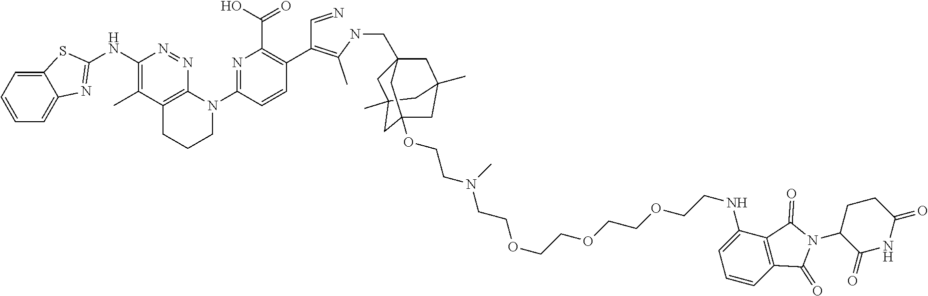 [SMILES: C/C(NCC12CC3(C)CC(C)(C1)CC(OCCN(C)CCOCCOCCOCCNc1cccc4c1C(=O)N(C1CCC(=O)NC1=O)C4=O)(C3)C2)=C(/C=N)c1ccc(N2CCCc3c2nnc(Nc2nc4ccccc4s2)c3C)nc1C(=O)O] |
| | 6-[3-(1,3-benzothiazol-2-ylamino)-4-methyl-6,7-dihydro-5H-pyrido[2,3-c] |
| | pyridazin-8-yl]-3-[1-[[3-[2-[2-[2-[2-[2-[[2-(2,6-dioxo-3-piperidyl)-1,3-dioxo- |
| | isoindolin-4-yl]amino]ethoxy]ethoxy]ethoxy]ethyl-methyl-amino]ethoxy]-5,7- |
| | dimethyl-1-adamantyl]methyl]-5-methyl-pyrazol-4-yl]pyridine-2-carboxylic |
| | acid |
| 12 | 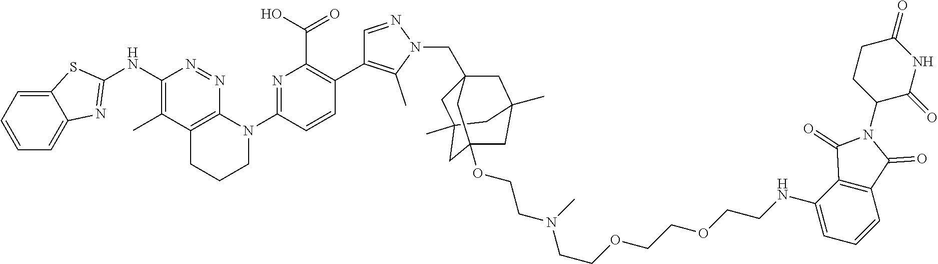 [SMILES: Cc1c(Nc2nc3ccccc3s2)nnc2c1CCCN2c1ccc(-c2cnn(CC34CC5(C)CC(C)(C3)CC(OCCN(C)CCOCCOCCNc3cccc6c3C(=O)N(C3CCC(=O)NC3=O)C6=O)(C5)C4)c2C)c(C(=O)O)n1] |
| | 6-[3-(1,3-benzothiazol-2-ylamino)-4-methyl-6,7-dihydro-5H-pyrido[2,3-c] |
| | pyridazin-8-yl]-3-[1-[[3-[2-[2-[2-[2-[[2-(2,6-dioxo-3-piperidyl)-1,3-dioxo- |
| | isoindolin-4-yl]amino]ethoxy]ethoxy]ethyl-methyl-amino]ethoxy]-5,7- |
| | dimethyl-1-adamantyl]methyl]-5-methyl-pyrazol-4-yl]pyridine-2-carboxylic |
| | acid |
| 13 | 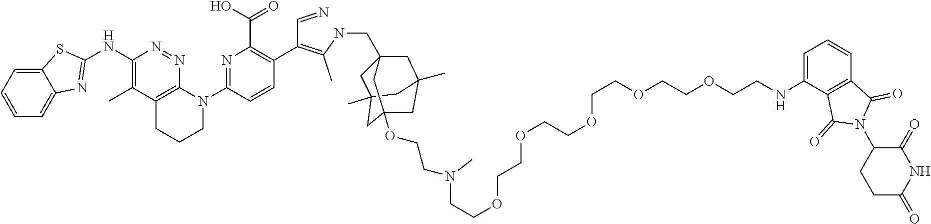 [SMILES: C/C(NCC12CC3(C)CC(C)(C1)CC(OCCN(C)CCOCCOCCOCCOCCOCCNc1cccc4c1C(=O)N(C1CCC(=O)NC1=O)C4=O)(C3)C2)=C(/C=N)c1ccc(N2CCCc3c2nnc(Nc2nc4ccccc4s2)c3C)nc1C(=O)O] |
| | 6-[3-(1,3-benzothiazol-2-ylamino)-4-methyl-6,7-dihydro-5H-pyrido[2,3-c] |
| | pyridazin-8-yl]-3-[1-[[3-[2-[2-[2-[2-[2-[2-[2-[[2-(2,6-dioxo-3-piperidyl)-1,3- |
| | dioxo-isoindolin-4-yl]amino]ethoxy]ethoxy]ethoxy]ethoxy]ethoxy]ethyl- |
| | methyl-amino]ethoxy]-5,7-dimethyl-1-adamantyl]methyl]-5-methyl-pyrazol- |
| | 4-yl]pyridine-2-carboxylic acid |
| 14 | 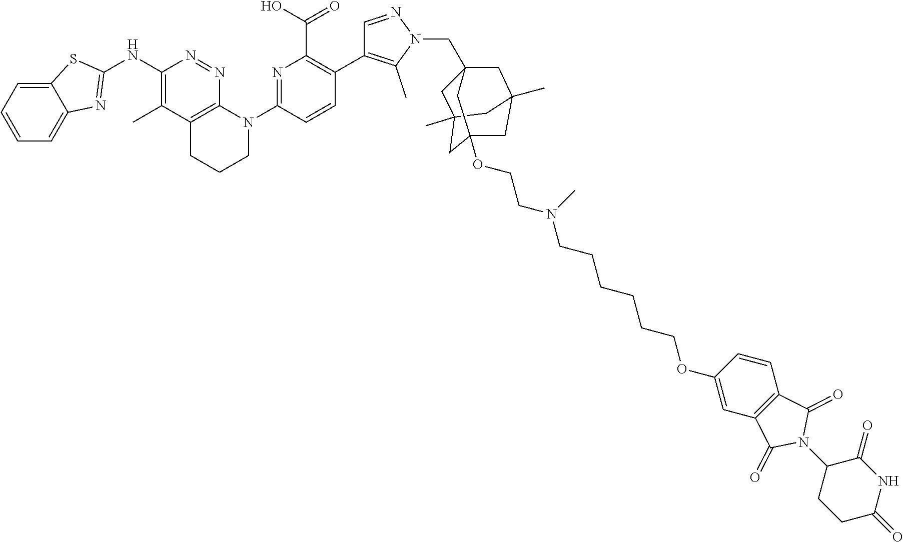 [SMILES: Cc1c(Nc2nc3ccccc3s2)nnc2c1CCCN2c1ccc(-c2cnn(CC34CC5(C)CC(C)(C3)CC(OCCN(C)CCCCCCOc3ccc6c(c3)C(=O)N(C3CCC(=O)NC3=O)C6=O)(C5)C4)c2C)c(C(=O)O)n1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[6-[2-(2,6-dioxo-3-piperidyl)-1,3-dioxo-isoindolin- |
| | 5-yl]oxy hexyl-methyl-amino]ethoxy]-5,7-dimethyl-1-adamantyl]methyl]-5- |
| | methyl-pyrazol-4-yl]pyridine-2-carboxylic acid |
| 15 | 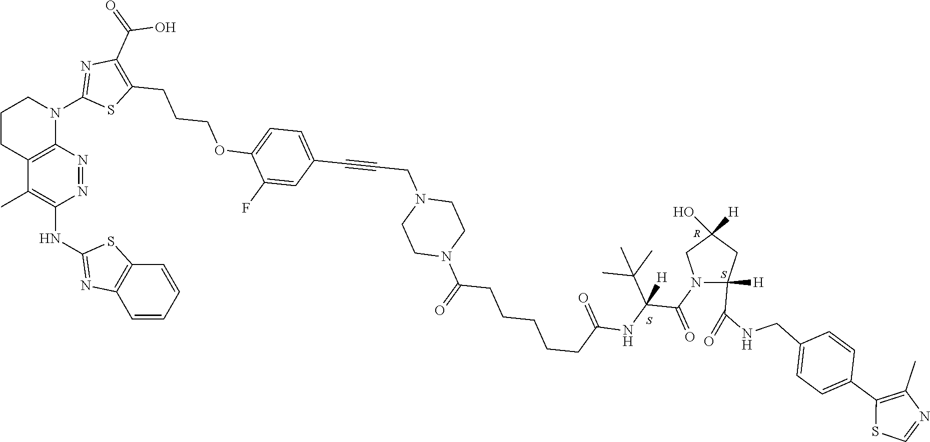 |
| | 2-[3-(1,3-benzothiazol-2-ylamino)-4-methyl-6,7-dihydro-5H-pyrido[2,3- |
| | c]pyridazin-8-yl]-5-[3-[2-fluoro-4-[3-[4-[7-[[(1S)-1-[(2S,4R)-4-hydroxy-2-[[4- |
| | (4-methylthiazol-5-yl)phenyl]methylcarbamoyl]pyrrolidine-1-carbonyl]-2,2- |
| | dimethyl-propyl]amino]-7-oxo-heptanoyl]piperazin-1-yl]prop-1- |
| | ynyl]phenoxy]propyl]thiazole-4-carboxylic acid |
| 16 | 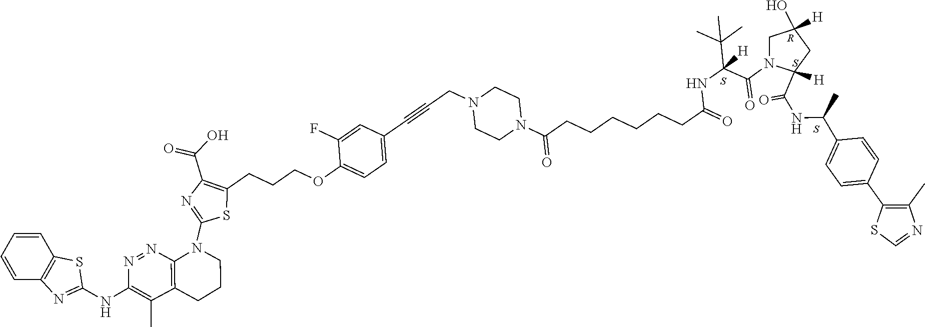 |
| | 2-[3-(1,3-benzothiazol-2-ylamino)-4-methyl-6,7-dihydro-5H-pyrido[2,3-c] |
| | pyridazin-8-yl]-5-[3-[2-fluoro-4-[3-[4-[8-[[(1S)-1-[[(2S,4R)-4-hydroxy-2-[[(1S)- |
| | 1-[4-(4-methyl thiazol-5-yl)phenyl]ethyl]carbamoyl]pyrrolidine-1-carbonyl]- |
| | 2,2-dimethyl-propyl]amino]-8-oxo-octanoyl]piperazin-1-yl]prop-1- |
| | ynyl]phenoxy]propyl]thiazole-4-carboxylic acid |
| 17 | 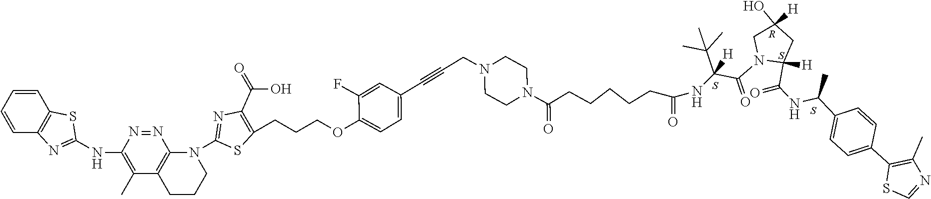 |
| | 2-[3-(1,3-benzothiazol-2-ylamino)-4-methyl-6,7-dihydro-5H-pyrido[2,3- |
| | c]pyridazin-8-yl]-5-[3-[2-fluoro-4-[3-[4-[7-[[(1S)-1-[(2S,4R)-4-hydroxy-2- |
| | [[(1S)-1-[4-(4-methylthiazol-5-yl)phenyl]ethyl]carbamoyl]pyrrolidine-1- |
| | carbonyl]-2,2-dimethyl-propyl]amino]-7-oxo-heptanoyl]piperazin-1-yl]prop- |
| | 1-ynyl]phenoxy]propyl]thiazole-4-carboxylic acid |
| 18 | 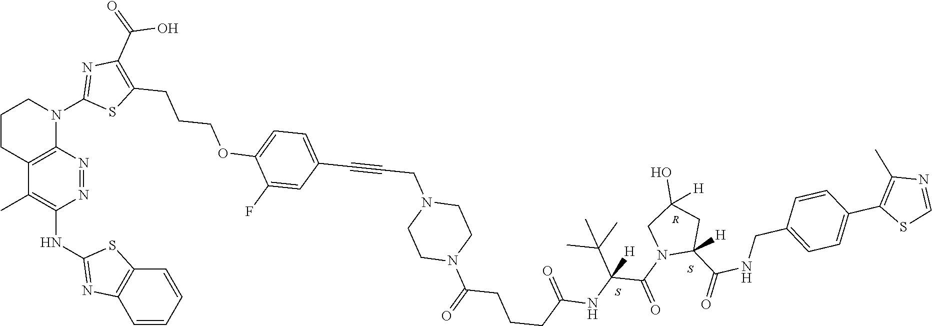 |
| | 2-[3-(1,3-benzothiazol-2-ylamino)-4-methyl-6,7-dihydro-5H-pyrido[2,3-c] |
| | pyridazin-8-yl]-5-[3-[2-fluoro-4-[3-[4-[5-[[(1S)-1-[(2S,4R)-4-hydroxy-2-[[4-(4- |
| | methylthiazol-5-yl)phenyl]methylcarbamoyl]pyrrolidine-1-carbonyl]-2,2- |
| | dimethyl-propyl]amino]-5-oxo-pentanoyl]piperazin-1-yl]prop-1- |
| | ynyl]phenoxylpropyl]thiazole-4-carboxylic acid |
| 19 | 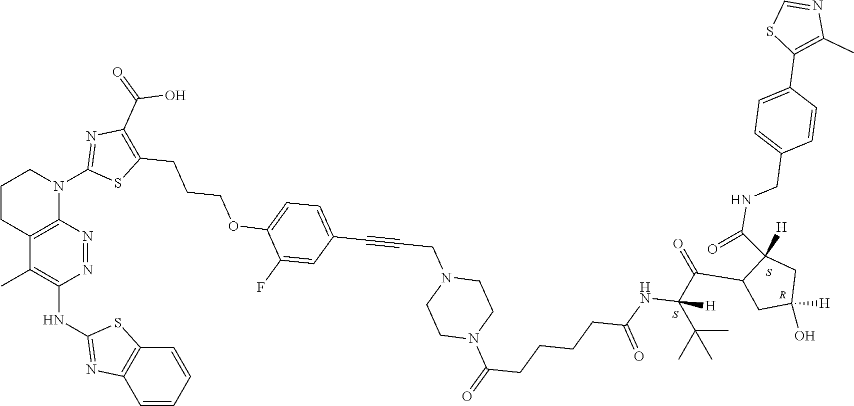 |
| | 2-[3-(1,3-benzothiazol-2-ylamino)-4-methyl-6,7-dihydro-5H-pyrido[2,3-c] |
| | pyridazin-8-yl]-5-[3-[2-fluoro-4-[3-[4-[6-[[(1S)-1-[(2S,4R)-4-hydroxy-2-[[4-(4- |
| | methylthiazol-5-yl)phenyl]methylcarbamoyl]pyrrolidine-1-carbonyl]-2,2- |
| | dimethyl-propyl]amino]-6-oxo-hexanoyl]piperazin-1-yl]prop-1- |
| | ynyl]phenoxy]propyl]thiazole-4-carboxylic acid |
| 20 | 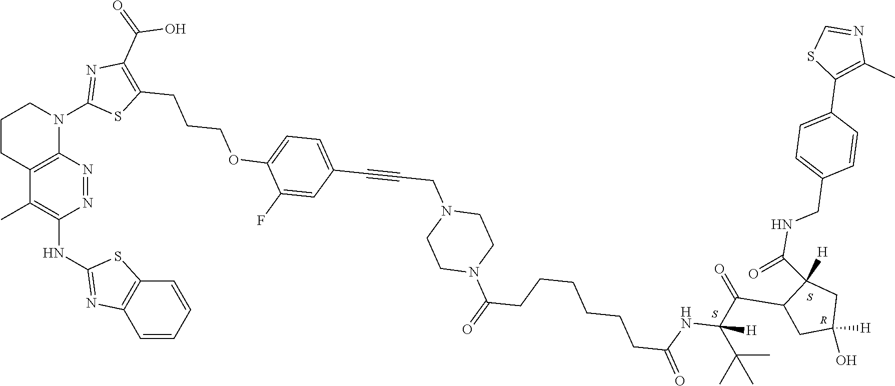 |
| | 2-[3-(1,3-benzothiazol-2-ylamino)-4-methyl-6,7-dihydro-5H-pyrido[2,3-c] |
| | pyridazin-8-yl]-5-[3-[2-fluoro-4-[3-[4-[8-[[(1S)-1-[(2S,4R)-4-hydroxy-2-[[4-(4- |
| | methylthiazol-5-yl)phenyl]methylcarbamoyl]pyrrolidine-1-carbonyl]-2,2- |
| | dimethyl-propyl]amino]-8-oxo-octanoyl]piperazin-1-yl]prop-1- |
| | ynyl]phenoxy]propyl]thiazole-4-carboxylic acid |
| 21 | 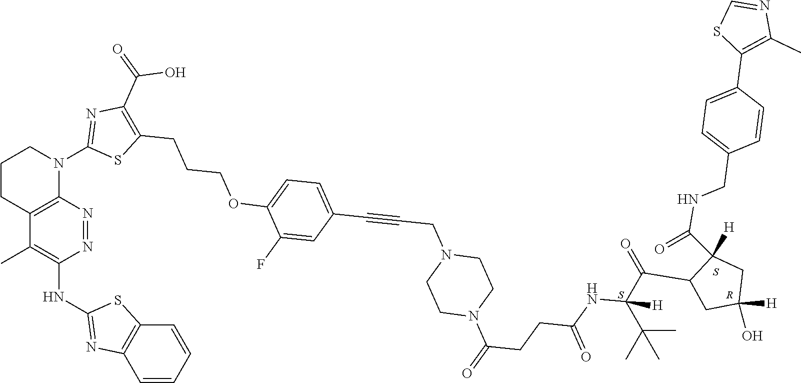 |
| | 2-[3-(1,3-benzothiazol-2-ylamino)-4-methyl-6,7-dihydro-5H-pyrido[2,3-c] |
| | pyridazin-8-yl]-5-[3-[2-fluoro-4-[3-[4-[4-[[(1S)-1-[(2S,4R)-4-hydroxy-2-[[4-(4- |
| | methylthiazol-5-yl)phenyl]methylcarbamoyl]pyrrolidine-1-carbonyl]-2,2- |
| | dimethyl-propyl]amino]-4-oxo-butanoyl]piperazin-1-yl]prop-1- |
| | ynyl]phenoxy]propyl]thiazole-4-carboxylic acid |
| 22 | 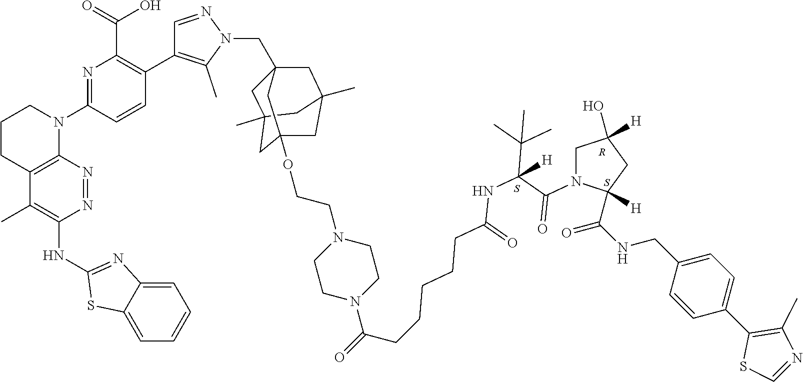 |
| | 6-[3-(1,3-benzothiazol-2-ylamino)-4-methyl-6,7-dihydro-5H-pyrido[2,3-c] |
| | pyridazin-8-yl]-3-[1-[3-[2-[4-[7-[[(1S)-1-[(2S,4R)-4-hydroxy-2-[[4-(4- |
| | methylthiazol-5-yl)phenyl]methylcarbamoyl]pyrrolidine-1-carbonyl]-2,2- |
| | dimethyl-propyl]amino]-7-oxo-heptanoyl]piperazin-1-yl]ethoxy]-5,7- |
| | dimethyl-1-adamantyl]methyl]-5-methyl-pyrazol-4-yl]pyridine-2-carboxylic |
| | acid |
| 23 | 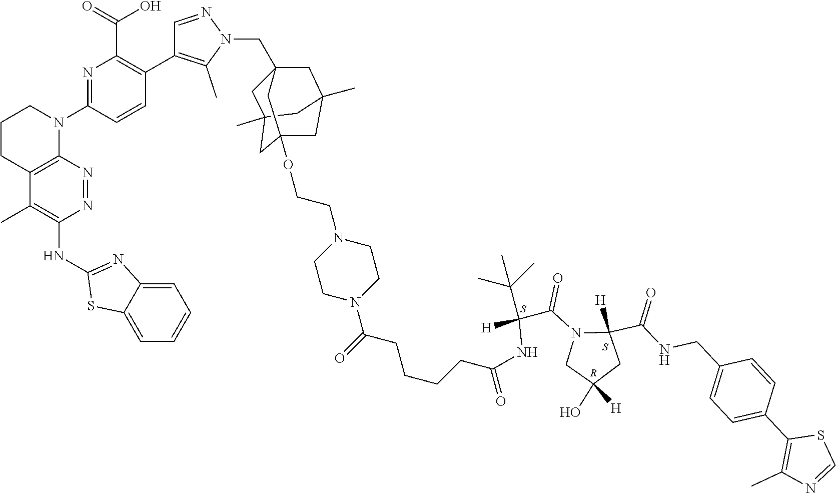 |
| | 6-[3-(1,3-benzothiazol-2-ylamino)-4-methyl-6,7-dihydro-5H-pyrido[2,3-c] |
| | pyridazin-8-yl]-3-[1-[[3-[2-[4-[6-[[(1S)-1-[(2S,4R)-4-hydroxy-2-[[4-(4- |
| | methylthiazol-5-yl)phenyl]methylcarbamoyl]pyrrolidine-1-carbonyl]-2,2- |
| | dimethyl-propyl]amino]-6-oxo-hexanoyl]piperazin-1-yl]ethoxy]-5,7-dimethyl- |
| | 1-adamantyl]methyl]-5-methyl-pyrazol-4-yl]pyridine-2-carboxylic acid |
| 24 | 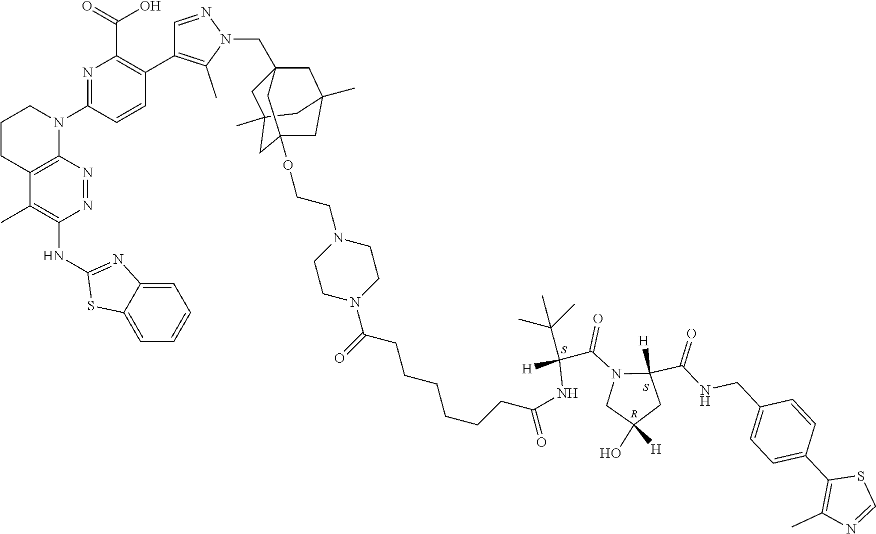 |
| | 6-[3-(1,3-benzothiazol-2-ylamino)-4-methyl-6,7-dihydro-5H-pyrido[2,3-c] |
| | pyridazin-8-yl]-3-[1-[[3-[2-[4-[8-[[(1S)-1-[(2S,4R)-4-hydroxy-2-[[4-(4- |
| | methylthiazol-5-yl)phenyl]methylcarbamoyl]pyrrolidine-1-carbonyl]-2,2- |
| | dimethyl-propyl]amino]-8-oxo-octanoyl]piperazin-1-yl]ethoxy]-5,7-dimethyl- |
| | 1-adamantyl]methyl]-5-methyl-pyrazol-4-yl]pyridine-2-carboxylic acid |
| 25 | 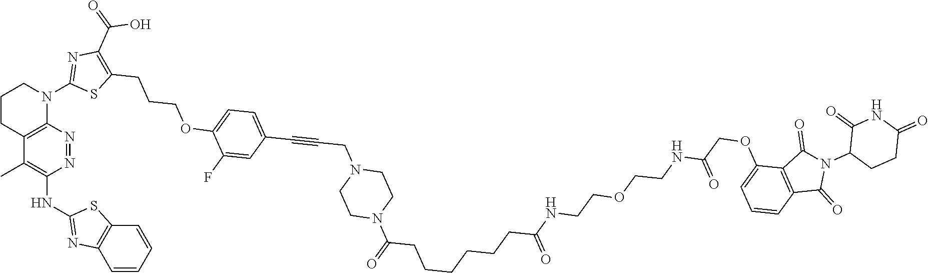 [SMILES: Cc1c(Nc2nc3ccccc3s2)nnc2c1CCCN2c1nc(C(=O)O)c(CCCOc2ccc(C#CCN3CCN(C(=O)CCCCCCC(=O)NCCOCCNC(=O)COc4cccc5c4C(=O)N(C4CCC(=O)NC4=O)C5=O)CC3)cc2F)s1] |
| | 2-[3-(1,3-benzothiazol-2-ylamino)-4-methyl-6,7-dihydro-5H-pyrido[2,3-c] |
| | pyridazin-8-yl]-5-[3-[4-[3-[4-[8-[2-[2-[[2-[2-(2,6-dioxo-3-piperidyl)-1,3-dioxo- |
| | isoindolin-4-yl]oxyacetyl]amino]ethoxy]ethylamino]-8-oxo- |
| | octanoyl]piperazin-1-yl]prop-1-ynyl]-2-fluoro-phenoxy]propyl]thiazole-4- |
| | carboxylic acid |
| 26 | 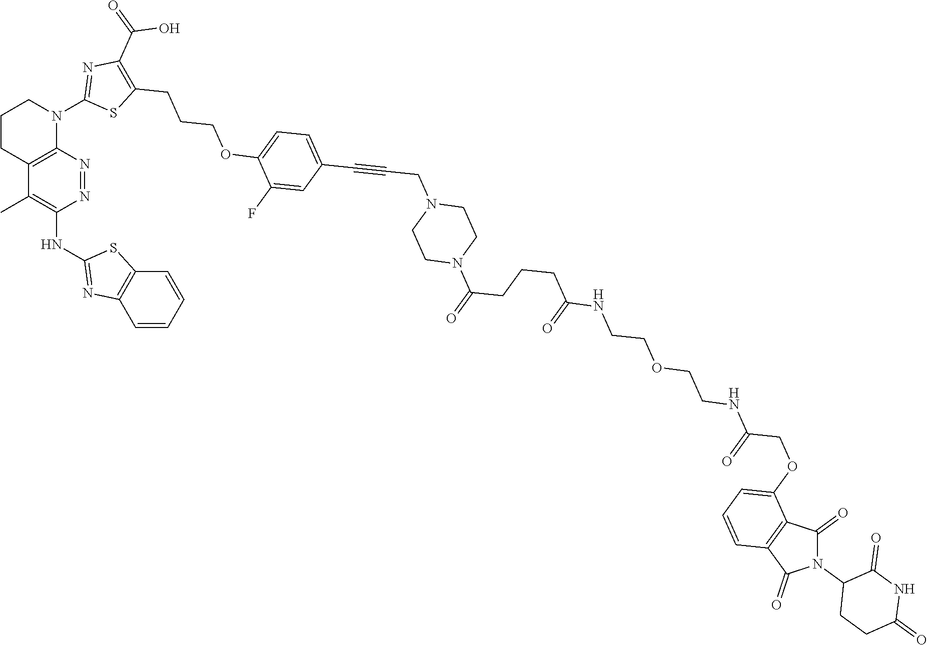 [SMILES: Cc1c(Nc2nc3ccccc3s2)nnc2c1CCCN2c1nc(C(=O)O)c(CCCOc2ccc(C#CCN3CCN(C(=O)CCCC(=O)NCCOCCNC(=O)COc4cccc5c4C(=O)N(C4CCC(=O)NC4=O)C5=O)CC3)cc2F)s1] |
| | 2-[3-(1,3-benzothiazol-2-ylamino)-4-methyl-6,7-dihydro-5H-pyrido[2,3-c] |
| | pyridazin-8-yl]-5-[3-[4-[3-[4-[5-[2-[2-[[2-[2-(2,6-dioxo-3-piperidyl)-1,3-dioxo- |
| | isoindolin-4-yl]oxyacetyl]amino]ethoxy]ethylamino]-5-oxo- |
| | pentanoyl]piperazin-1-yl]prop-1-ynyl]-2-fluoro-phenoxy]propyl]thiazole-4- |
| | carboxylic acid |
| 27 | 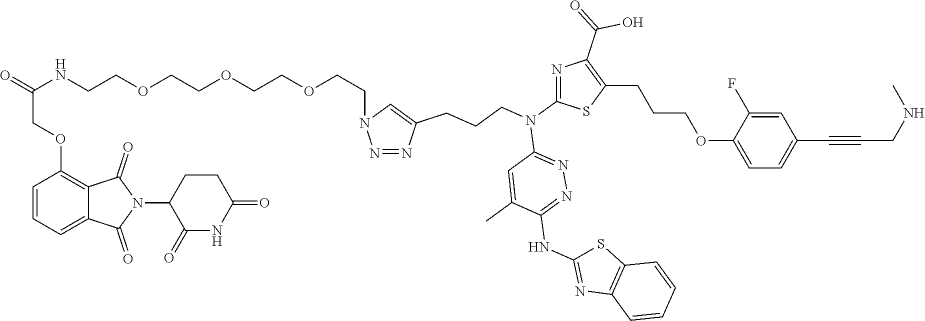 [SMILES: CNCC#Cc1ccc(OCCCc2sc(N(CCCc3cn(CCOCCOCCOCCNC(=O)COc4cccc5c4C(=O)N(C4CCC(=O)NC4=O)C5=O)nn3)c3cc(C)c(Nc4nc5ccccc5s4)nn3)nc2C(=O)O)c(F)c1] |
| | 2-[[6-(1,3-benzothiazol-2-ylamino)-5-methyl-pyridazin-3-yl]-[3-[1-[2-[2-[2-[2- |
| | [[2-[2-(2,6-dioxo-3-piperidyl)-1,3-dioxo-isoindolin-4-yl]oxyacetyl]amino] |
| | ethoxy]ethoxy]ethoxy]ethyl]triazol-4-yl]propyl]amino]-5-[3-[2-fluoro-4-[3- |
| | (methylamino)prop-1-ynyl]phenoxy]propyl]thiazole-4-carboxylic acid |
| 28 | 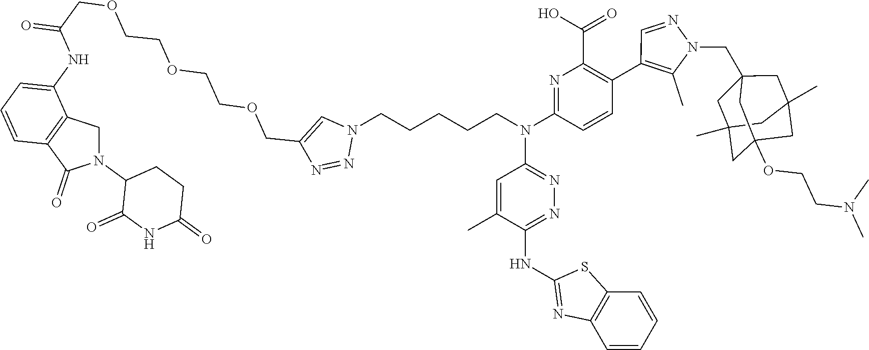 [SMILES: Cc1cc(N(CCCCCn2cc(COCCOCCOCC(=O)Nc3cccc4c3CN(C3CCC(=O)NC3=O)C4=O)nn2)c2ccc(-c3cnn(CC45CC6(C)CC(C)(C4)CC(OCCN(C)C)(C6)C5)c3C)c(C(=O)O)n2)nnc1Nc1nc2ccccc2s1] |
| | 6-[6-(1,3-benzothiazol-2-ylamino)-5-methyl-pyridazin-3-yl]-[5-[4-[2-[2-[2-[[2- |
| | (2,6-dioxo-3-piperidyl)-1-oxo-isoindolin-4-yl]amino]-2-oxo-ethoxy]ethoxy] |
| | ethoxymethyl]triazol-1-yl]pentyl]amino]-3-[1-[3-[2-(dimethylamino)ethoxy]- |
| | 5,7-dimethyl-1-adamantyl]methyl]-5-methyl-pyrazol-4-yl]pyridine-2- |
| | carboxylic acid |
| 29 | 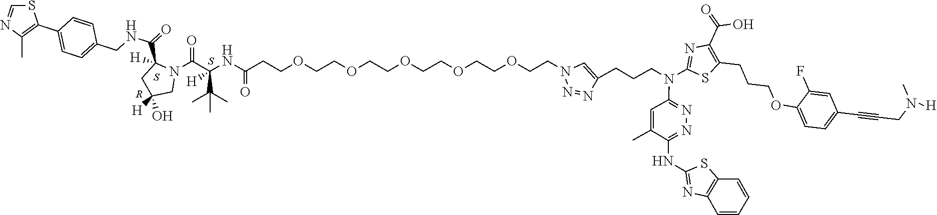 |
| | 2-[[6-(1,3-benzothiazol-2-ylamino)-5-methyl-pyridazin-3-yl]-[3-[1-[2-[2-[2-[2- |
| | [2-[3-[[(1S)-1-[(2S,4R)-4-hydroxy-2-[[4-(4-methylthiazol-5-yl)phenyl]methyl |
| | carbamoyl]pyrrolidine-1-carbonyl]-2,2-dimethyl-propyl]amino]-3-oxo- |
| | propoxy]ethoxy]ethoxy]ethoxy]ethoxy]ethyl]triazol-4-yl]propyl]amino]-5-[3- |
| | [2-fluoro-4-[3-(methylamino)prop-1-ynyl]phenoxy]propyl]thiazole-4- |
| | carboxylic acid |
| 30 | 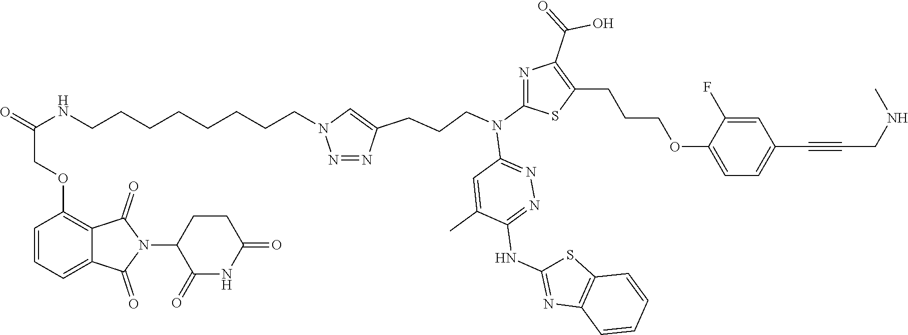 [SMILES: CNCC#Cc1ccc(OCCCc2sc(N(CCCc3cn(CCCCCCCCNC(=O)COc4cccc5c4C(=O)N(C4CCC(=O)NC4=O)C5=O)nn3)c3cc(C)c(Nc4nc5ccccc5s4)nn3)nc2C(=O)O)c(F)c1] |
| | 2-[[6-(1,3-benzothiazol-2-ylamino)-5-methyl-pyridazin-3-yl]-[3-[1-[8-[2-[2- |
| | (2,6-dioxo-3-piperidyl)-1,3-dioxo-isoindolin-4-yl]oxyacetyl]amino]octyl] |
| | triazol-4-yl]propyl]amino]-5-[3-[2-fluoro-4-[3-(methylamino)prop-1- |
| | ynyl]phenoxy]propyl]thiazole-4-carboxylic acid |
| 31 | 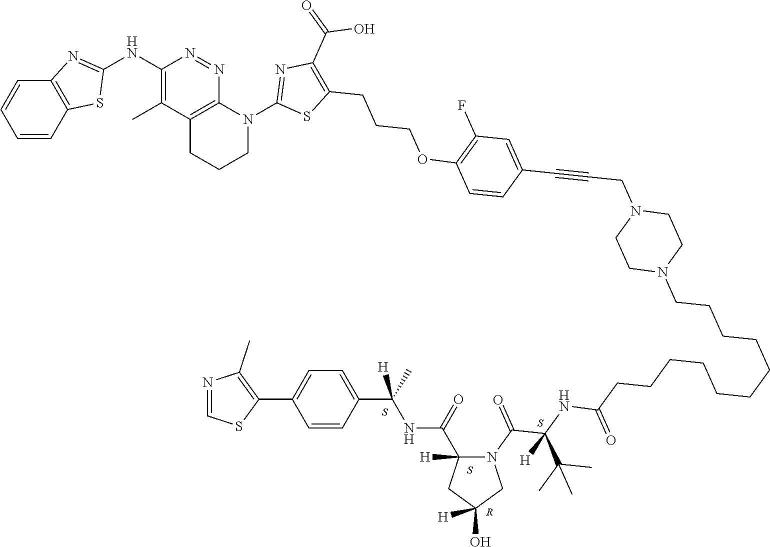 |
| | 2-[3-(1,3-benzothiazol-2-ylamino)-4-methyl-6,7-dihydro-5H-pyrido[2,3-c] |
| | pyridazin-8-yl]-5-[3-[2-fluoro-4-[3-[4-[12-[[(1S)-1-[(2S,4R)-4-hydroxy-2- |
| | [[(1S)-1-[4-(4-methylthiazol-5-yl)phenyl]ethyl]carbamoyl]pyrrolidine-1- |
| | carbonyl]-2,2-dimethyl-propyl]amino]-12-oxo-dodecyl]piperazin-1-yl]prop-1- |
| | ynyl]phenoxy]propyl]thiazole-4-carboxylic acid |
| 32 | 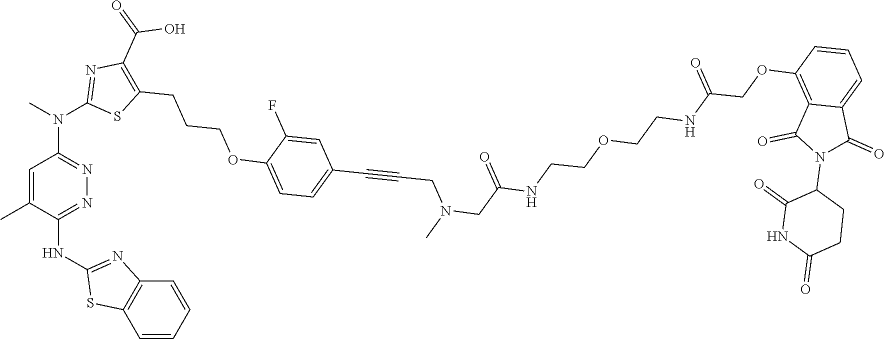 [SMILES: Cc1cc(N(C)c2nc(C(=O)O)c(CCCOc3ccc(C#CCN(C)CC(=O)NCCOCCNC(=O)COc4cccc5c4C(=O)N(C4CCC(=O)NC4=O)C5=O)cc3F)s2)nnc1Nc1nc2ccccc2s1] |
| | 2-[[6-(1,3-benzothiazol-2-ylamino)-5-methyl-pyridazin-3-yl]-methyl-amino]- |
| | 5-[3-[4-[3-[[2-[2-[2-[[2-[2-(2,6-dioxo-3-piperidyl)-1,3-dioxo-isoindolin-4- |
| | yl]oxyacetyl]amino]ethoxy]ethylamino]-2-oxo-ethyl]-methyl-amino]prop-1- |
| | ynyl]-2-fluoro-phenoxy]propyl]thiazole-4-carboxylic acid |
| 33 | 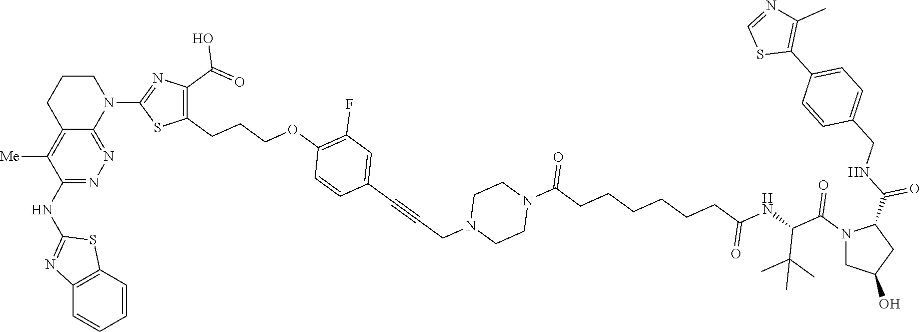 [SMILES: Cc1ncsc1-c1ccc(CNC(=O)[C@@H]2C[C@@H](O)CN2C(=O)[C@@H](NC(=O)CCCCCCC(=O)N2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(8-(((S)-1-((2S,4R)-4-hydroxy-2- |
| | ((4-(4-methylthiazol-5-yl)benzyl)carbamoyl)pyrrolidin-1-yl)-3,3-dimethyl-1- |
| | oxobutan-2-yl)amino)-8-oxo octanoyl)piperazin-1-yl)prop-1-yn-1- |
| | yl)phenoxy)propyl)thiazole-4-carboxylic acid |
| 34 | 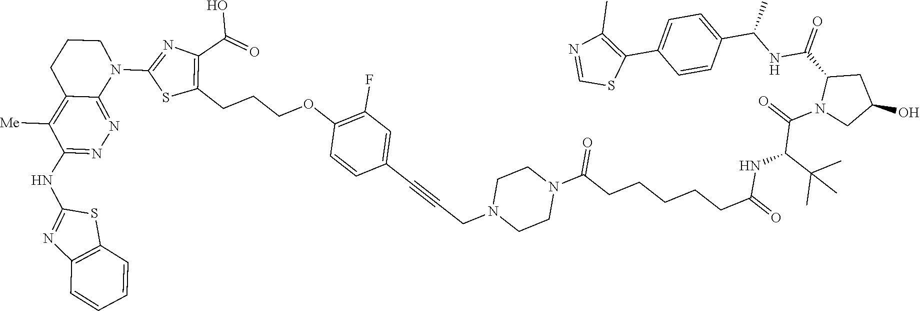 [SMILES: Cc1ncsc1-c1ccc([C@H](C)NC(=O)[C@@H]2C[C@@H](O)CN2C(=O)[C@@H](NC(=O)CCCCCC(=O)N2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(7-(((S)-1-((2S,4R)-4-hydroxy-2- |
| | (((S)-1-(4-(4-methylthiazol-5-yl)phenyl)ethyl)carbamoyl)pyrrolidin-1-yl)-3,3- |
| | dimethyl-1-oxobutan-2-yl)amino)-7-oxoheptanoyl)piperazin-1-yl)prop-1-yn- |
| | 1-yl)phenoxy)propyl)thiazole-4- |
| | carboxylic acid |
| 35 | 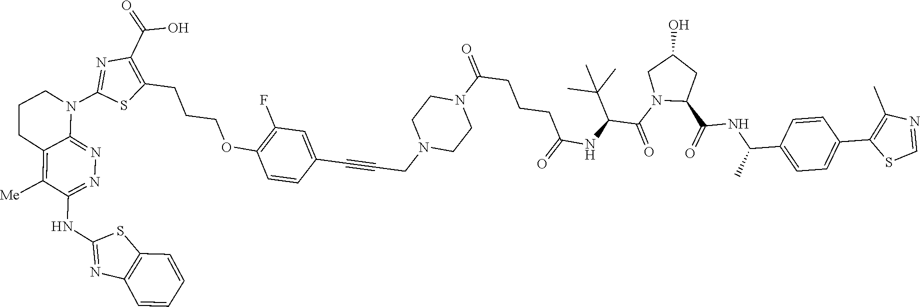 [SMILES: Cc1ncsc1-c1ccc([C@H](C)NC(=O)[C@@H]2C[C@@H](O)CN2C(=O)[C@@H](NC(=O)CCCC(=O)N2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(5-(((S)-1-((2S,4R)-4-hydroxy-2- |
| | (((S)-1-(4-(4-methylthiazol-5-yl)phenyl)ethyl)carbamoyl)pyrrolidin-1-yl)-3,3- |
| | dimethyl-1-oxobutan-2-yl)amino)-5-oxopentanoyl)piperazin-1-yl)prop-1-yn- |
| | 1-yl)phenoxy)propyl)thiazole-4-carboxylic acid |
| 36 | 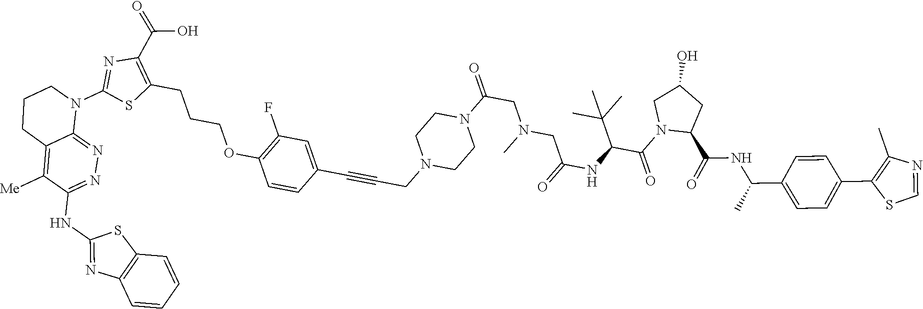 [SMILES: Cc1ncsc1-c1ccc([C@H](C)NC(=O)[C@@H]2C[C@@H](O)CN2C(=O)[C@@H](NC(=O)CN(C)CC(=O)N2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(N-(2-(((S)-1-((2S,4R)-4- |
| | hydroxy-2-(((S)-1-(4-(4-methylthiazol-5- |
| | yl)phenyl)ethyl)carbamoyl)pyrrolidin-1-yl)-3,3-dimethyl-1-oxobutan-2- |
| | yl)amino)-2-oxoethyl)-N-methylglycyl)piperazin-1-yl)prop-1-yn-1- |
| | yl)phenoxy)propyl)thiazole-4-carboxylic acid |
| 37 | 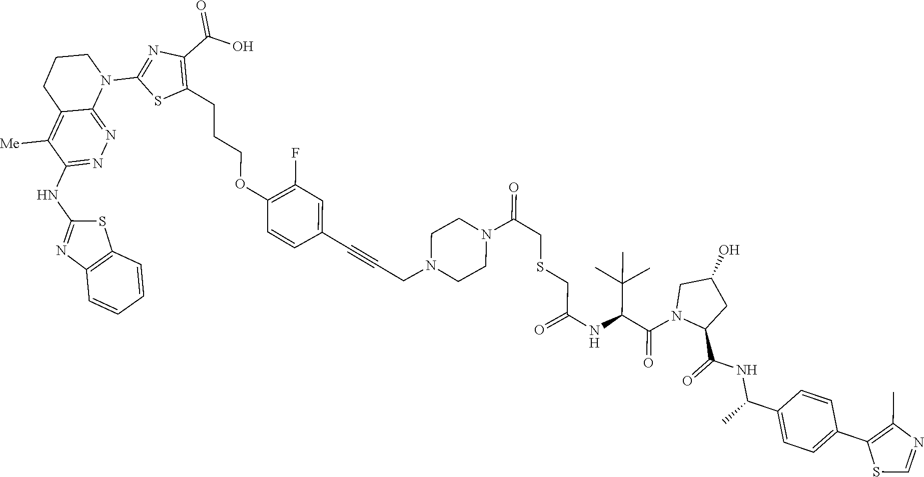 [SMILES: Cc1ncsc1-c1ccc([C@H](C)NC(=O)[C@@H]2C[C@@H](O)CN2C(=O)[C@@H](NC(=O)CSCC(=O)N2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(2-((2-(((S)-1-((2S,4R)-4- |
| | hydroxy-2-(((S)-1-(4-(4-methylthiazol-5- |
| | yl)phenyl)ethyl)carbamoyl)pyrrolidin-1-yl)-3,3-dimethyl-1-oxobutan-2- |
| | yl)amino)-2-oxoethyl)thio)acetyl)piperazin-1-yl)prop-1-yn-1- |
| | yl)phenoxy)propyl)thiazole-4-carboxylic acid |
| 38 | 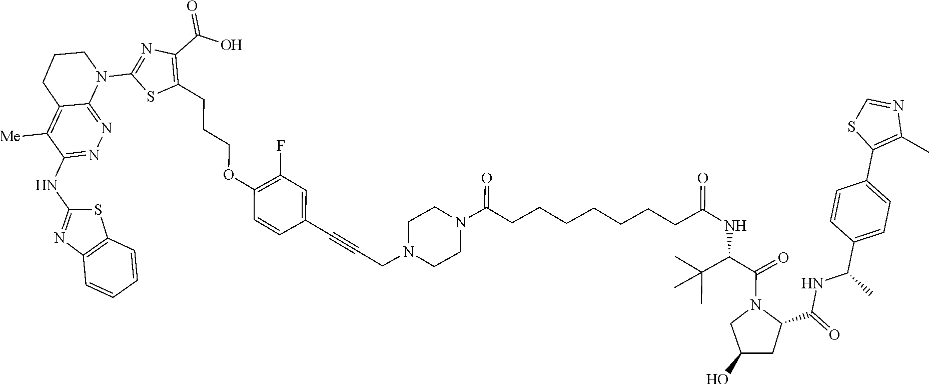 [SMILES: Cc1ncsc1-c1ccc([C@H](C)NC(=O)[C@@H]2C[C@@H](O)CN2C(=O)[C@@H](NC(=O)CCCCCCCC(=O)N2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(9-(((S)-1-((2S,4R)-4-hydroxy-2- |
| | (((S)-1-(4-(4-methylthiazol-5-yl)phenyl)ethyl)carbamoyl)pyrrolidin-1-yl)-3,3- |
| | dimethyl-1-oxobutan-2-yl)amino)-9-oxononanoyl)piperazin-1-yl)prop-1-yn- |
| | 1-yl)phenoxy)propyl)thiazole-4-carboxylic acid |
| 39 | 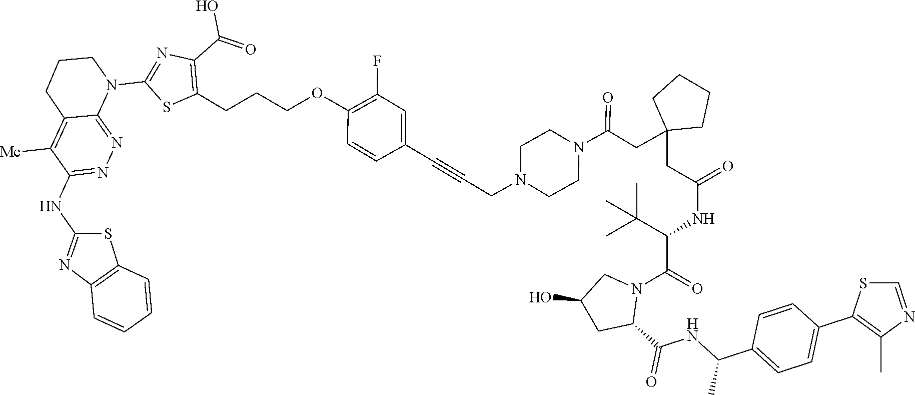 [SMILES: Cc1ncsc1-c1ccc([C@H](C)NC(=O)[C@@H]2C[C@@H](O)CN2C(=O)[C@@H](NC(=O)CC2(CC(=O)N3CCN(CC#Cc4ccc(OCCCc5sc(N6CCCc7c6nnc(Nc6nc8ccccc8s6)c7C)nc5C(=O)O)c(F)c4)CC3)CCCC2)C(C)(C)C)cc1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(2-(1-(2-(((S)-1-((2S,4R)-4- |
| | hydroxy-2-(((S)-1-(4-(4-methylthiazol-5- |
| | yl)phenyl)ethyl)carbamoyl)pyrrolidin-1-yl)-3,3-dimethyl-1-oxobutan-2- |
| | yl)amino)-2-oxoethyl)cyclopentyl)acetyl)piperazin-1-yl)prop-1-yn-1- |
| | yl)phenoxy)propyl)thiazole-4-carboxylic acid |
| 40 | 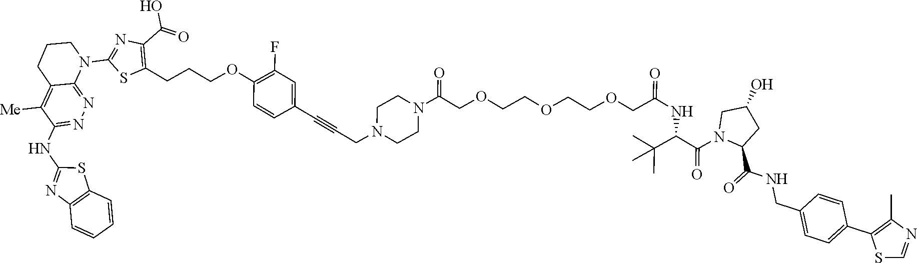 [SMILES: Cc1ncsc1-c1ccc(CNC(=O)[C@@H]2C[C@@H](O)CN2C(=O)[C@@H](NC(=O)COCCOCCOCC(=O)N2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-((S)-13-((2S,4R)-4-hydroxy-2- |
| | ((4-(4-methyl thiazol-5-yl)benzyl)carbamoyl)pyrrolidine-1-carbonyl)-14,14- |
| | dimethyl-11-oxo-3,6,9-trioxa-12-azapentadecanoyl)piperazin-1-yl)prop-1- |
| | yn-1-yl)phenoxy)propyl)thiazole-4-carboxylic acid |
| 41 | 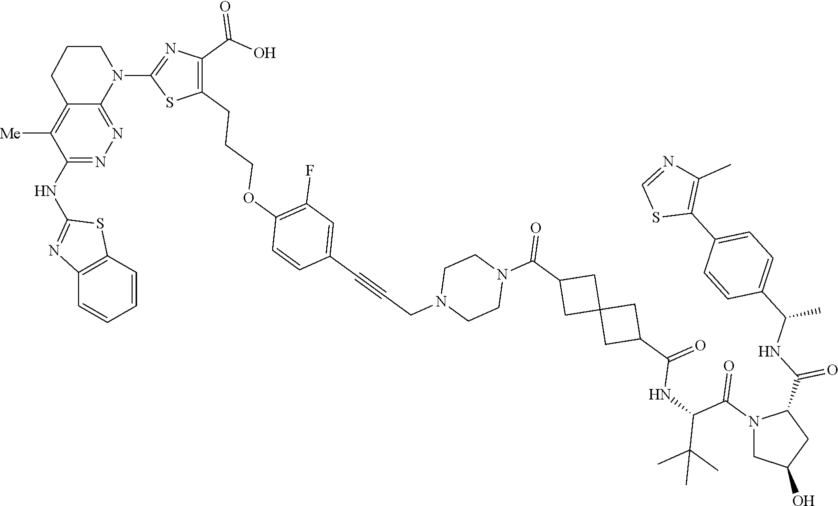 [SMILES: Cc1ncsc1-c1ccc([C@H](C)NC(=O)[C@@H]2C[C@@H](O)CN2C(=O)[C@@H](NC(=O)C2CC3(C2)CC(C(=O)N2CCN(CC#Cc4ccc(OCCCc5sc(N6CCCc7c6nnc(Nc6nc8ccccc8s6)c7C)nc5C(=O)O)c(F)c4)CC2)C3)C(C)(C)C)cc1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(6-(((S)-1-((2S,4R)-4-hydroxy-2- |
| | (((S)-1-(4-(4-methylthiazol-5-yl)phenyl)ethyl)carbamoyl)pyrrolidin-1-yl)-3,3- |
| | dimethyl-1-oxobutan-2-yl)carbamoyl)spiro[3.3]heptane-2- |
| | carbonyl)piperazin-1-yl)prop-1-yn-1-yl)phenoxy)propyl)thiazole-4- |
| | carboxylic acid |
| 42 | 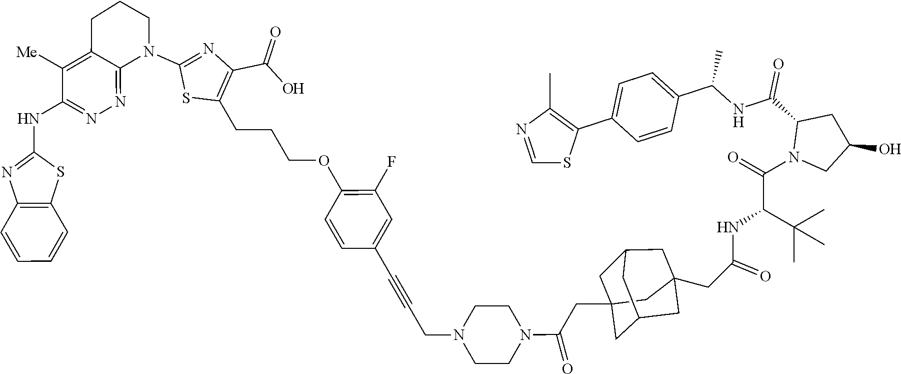 [SMILES: Cc1ncsc1-c1ccc([C@H](C)NC(=O)[C@@H]2C[C@@H](O)CN2C(=O)[C@@H](NC(=O)CC23CC4CC(C2)CC(CC(=O)N2CCN(CC#Cc5ccc(OCCCc6sc(N7CCCc8c7nnc(Nc7nc9ccccc9s7)c8C)nc6C(=O)O)c(F)c5)CC2)(C4)C3)C(C)(C)C)cc1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(2-((3S,5R)-3-(2-(((S)-1- |
| | ((2S,4R)-4-hydroxy-2-(((S)-1-(4-(4-methylthiazol-5- |
| | yl)phenyl)ethyl)carbamoyl)pyrrolidin-1-yl)-3,3-dimethyl-1-oxobutan-2- |
| | yl)amino)-2-oxoethyl)adamantan-1-yl)acetyl)piperazin-1-yl)prop-1-yn-1-yl) |
| | phenoxy)propyl)thiazole-4-carboxylic acid |
| 43 | 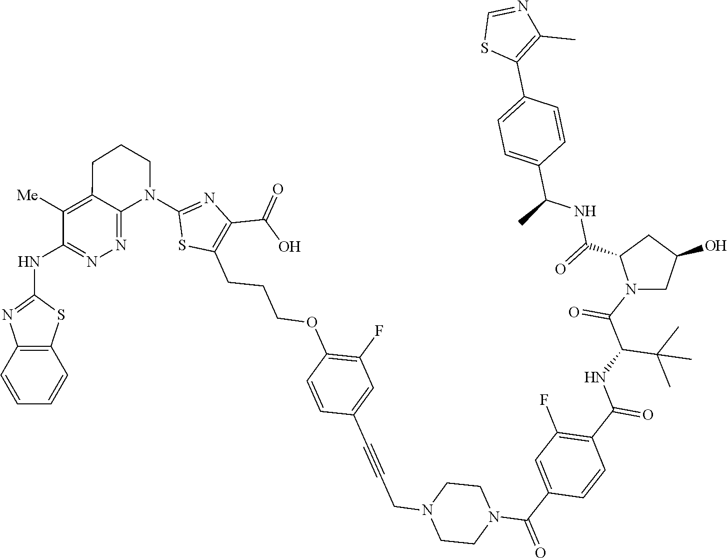 [SMILES: Cc1ncsc1-c1ccc([C@H](C)NC(=O)[C@@H]2C[C@@H](O)CN2C(=O)[C@@H](NC(=O)c2ccc(C(=O)N3CCN(CC#Cc4ccc(OCCCc5sc(N6CCCc7c6nnc(Nc6nc8ccccc8s6)c7C)nc5C(=O)O)c(F)c4)CC3)cc2F)C(C)(C)C)cc1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(3-fluoro-4-(((S)-1-((2S,4R)-4- |
| | hydroxy-2-(((S)-1-(4-(4-methylthiazol-5- |
| | yl)phenyl)ethyl)carbamoyl)pyrrolidin-1-yl)-3,3-dimethyl-1-oxobutan-2- |
| | yl)carbamoyl)benzoyl)piperazin-1-yl)prop-1-yn-1- |
| | yl)phenoxy)propyl)thiazole-4-carboxylic acid |
| 44 | 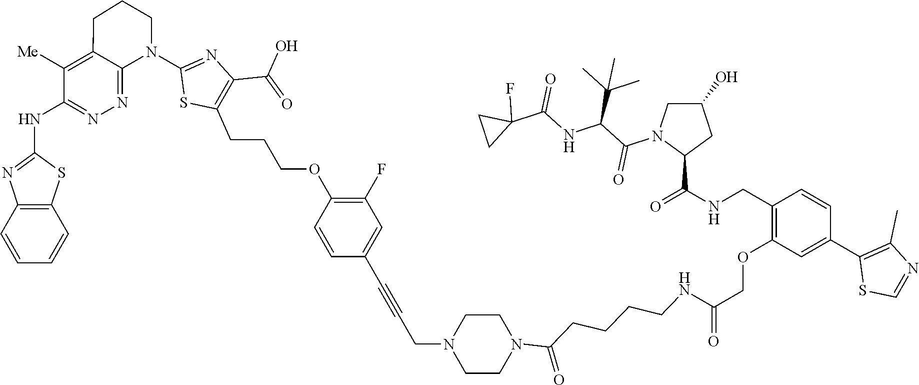 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCC(=O)N2CCN(CC#Cc3ccc(OCCCc4sc(N5CCCc6c5nnc(Nc5nc7ccccc7s5)c6C)nc4C(=O)O)c(F)c3)CC2)c1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(5-(2-(2-(((2S,4R)-1-((S)-2-(1- |
| | fluorocyclo propane-1-carboxamido)-3,3-dimethylbutanoyl)-4- |
| | hydroxypyrrolidine-2-carboxamido)methyl)-5-(4-methylthiazol-5- |
| | yl)phenoxy)acetamido)pentanoyl)piperazin-1-yl)prop-1-yn-1- |
| | yl)phenoxy)propyl)thiazole-4-carboxylic acid |
| 45 | 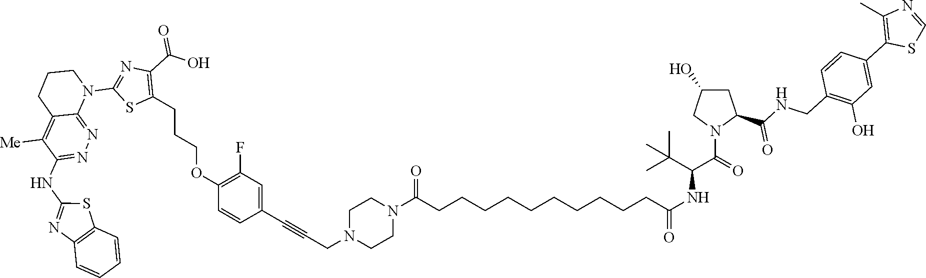 [SMILES: Cc1ncsc1-c1ccc(CNC(=O)[C@@H]2C[C@@H](O)CN2C(=O)[C@@H](NC(=O)CCCCCCCCCCC(=O)N2CCN(CC#Cc3ccc(OCCCc4sc(N5CCCc6c5nnc(Nc5nc7ccccc7s5)c6C)nc4C(=O)O)c(F)c3)CC2)C(C)(C)C)c(O)c1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(12-(((S)-1-((2S,4R)-4-hydroxy- |
| | 2-((2-hydroxy-4-(4-methylthiazol-5-yl)benzyl)carbamoyl)pyrrolidin-1-yl)-3,3- |
| | dimethyl-1-oxobutan-2-yl)amino)-12-oxododecanoyl)piperazin-1-yl)prop-1- |
| | yn-1-yl)phenoxy)propyl)thiazole-4-carboxylic acid |
| 46 | 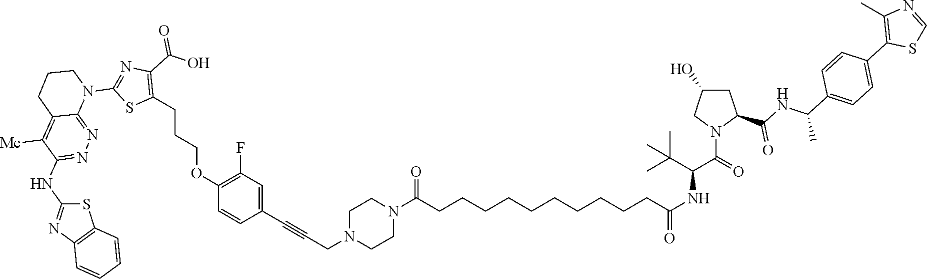 [SMILES: Cc1ncsc1-c1ccc([C@H](C)NC(=O)[C@@H]2C[C@@H](O)CN2C(=O)[C@@H](NC(=O)CCCCCCCCCCC(=O)N2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1] |
| | 2-(3-(benzo[d]thiazol-2-ylamino)-4-methyl-6,7-dihydro pyrido[2,3- |
| | c]pyridazin-8(5H)-yl)-5-(3-(2-fluoro-4-(3-(4-(12-(((S)-1-((2S,4R)-4-hydroxy- |
| | 2-(((S)-1-(4-(4-methylthiazol-5-yl)phenyl)ethyl)carbamoyl)pyrrolidin-1-yl)- |
| | 3,3-dimethyl-1-oxobutan-2-yl)amino)-12-oxododecanoyl)piperazin-1- |
| | yl)prop-1-yn-1-yl)phenoxy)propyl)thiazole-4-carboxylic acid |
| 47 | 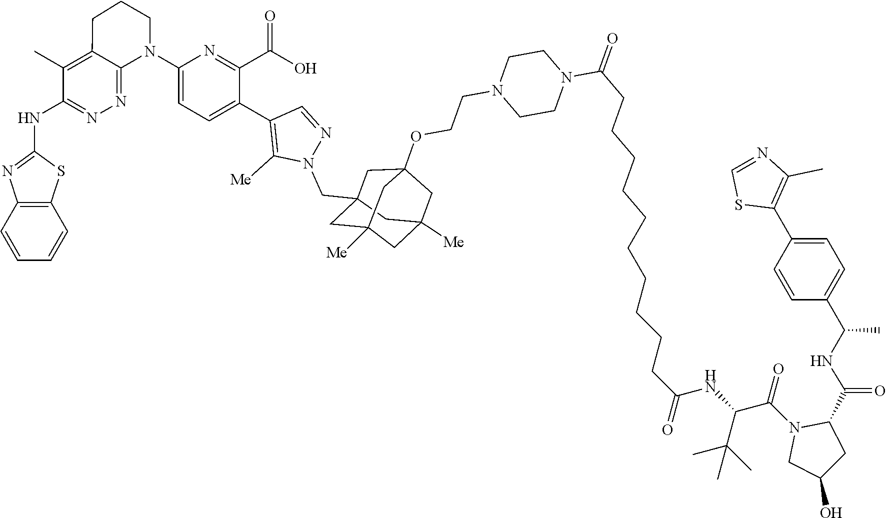 [SMILES: Cc1ncsc1-c1ccc([C@H](C)NC(=O)[C@@H]2C[C@@H](O)CN2C(=O)[C@@H](NC(=O)CCCCCCCCCCC(=O)N2CCN(CCOC34CC5(C)CC(C)(CC(Cn6ncc(-c7ccc(N8CCCc9c8nnc(Nc8nc%10ccccc%10s8)c9C)nc7C(=O)O)c6C)(C5)C3)C4)CC2)C(C)(C)C)cc1] |
| | 6-(3-(benzo[d]thiazol-2-ylamino)-4-methyl-6,7-dihydropyrido[2,3- |
| | c]pyridazin-8(5H)-yl)-3-(1-(((1r,3s,5R,7S)-3-(2-(4-(12-(((S)-1-((2S,4R)-4- |
| | hydroxy-2-(((S)-1-(4-(4-methylthiazol-5- |
| | yl)phenyl)ethyl)carbamoyl)pyrrolidin-1-yl)-3,3-dimethyl-1-oxo butan-2- |
| | yl)amino)-12-oxododecanoyl)piperazin-1-yl)ethoxy)-5,7- |
| | dimethyladamantan-1-yl)methyl)-5-methyl-1H-pyrazol-4-yl)picolinic acid |
| 48 | 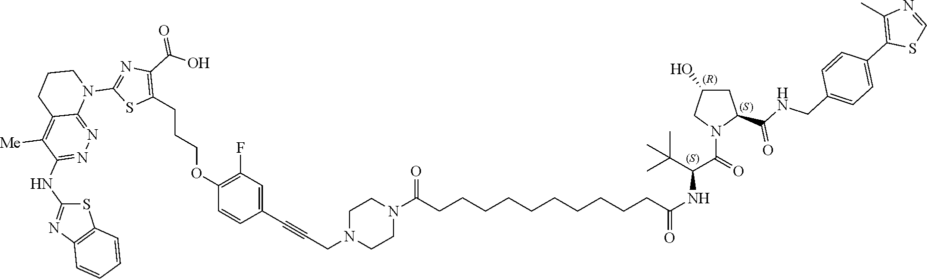 |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(12-(((S)-1-((2S,4R)-4-hydroxy- |
| | 2-((4-(4-methylthiazol-5-yl)benzyl)carbamoyl)pyrrolidin-1-yl)-3,3-dimethyl-1- |
| | oxobutan-2-yl)amino)-12-oxododecanoyl)piperazin-1-yl)prop-1-yn-1- |
| | yl)phenoxy)propyl)thiazole-4-carboxylic acid |
| 49 | 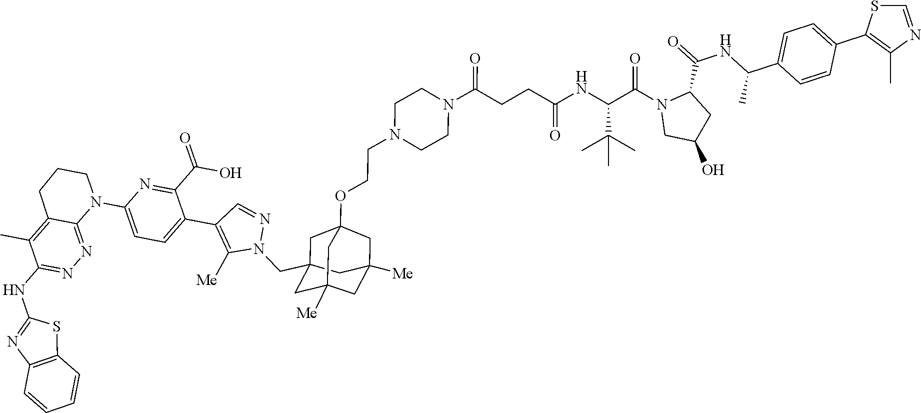 [SMILES: Cc1ncsc1-c1ccc([C@H](C)NC(=O)[C@@H]2C[C@@H](O)CN2C(=O)[C@@H](NC(=O)CCC(=O)N2CCN(CCOC34CC5(C)CC(C)(CC(Cn6ncc(-c7ccc(N8CCCc9c8nnc(Nc8nc%10ccccc%10s8)c9C)nc7C(=O)O)c6C)(C5)C3)C4)CC2)C(C)(C)C)cc1] |
| | 6-(3-(benzo[d]thiazol-2-ylamino)-4-methyl-6,7-dihydropyrido[2,3- |
| | c]pyridazin-8(5H)-yl)-3-(1-(((1r,3s,5R,7S)-3-(2-(4-(4-(((S)-1-((2S,4R)-4- |
| | hydroxy-2-(((S)-1-(4-(4-methylthiazol-5- |
| | yl)phenyl)ethyl)carbamoyl)pyrrolidin-1-yl)-3,3-dimethyl-1-oxobutan-2- |
| | yl)amino)-4-oxobutanoyl)piperazin-1-yl)ethoxy)-5,7-dimethyladamantan-1- |
| | yl)methyl)-5-methyl-1H-pyrazol-4-yl)picolinic acid |
| 50 | 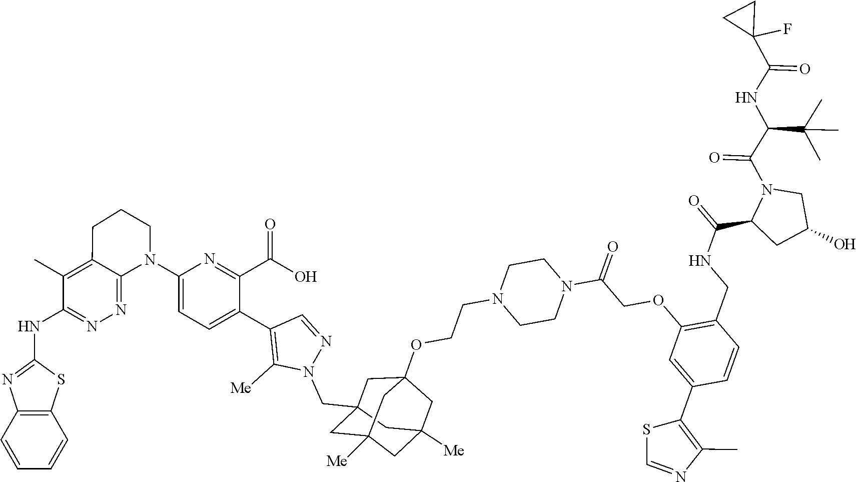 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)N2CCN(CCOC34CC5(C)CC(C)(CC(Cn6ncc(-c7ccc(N8CCCc9c8nnc(Nc8nc%10ccccc%10s8)c9C)nc7C(=O)O)c6C)(C5)C3)C4)CC2)c1] |
| | 6-(3-(benzo[d]thiazol-2-ylamino)-4-methyl-6,7-dihydropyrido[2,3- |
| | c]pyridazin-8(5H)-yl)-3-(1-(((1r,3s,5R,7S)-3-(2-(4-(2-(2-(((2S,4R)-1-((S)-2- |
| | (1-fluoro cyclopropane-1-carboxamido)-3,3-dimethylbutanoyl)-4- |
| | hydroxypyrrolidine-2-carboxamido)methyl)-5-(4-methylthiazol-5- |
| | yl)phenoxy)acetyl)piperazin-1-yl)ethoxy)-5,7-dimethyl adamantan-1- |
| | yl)methyl)-5-methyl-1H-pyrazol-4-yl)picolinic acid |
| 51 | 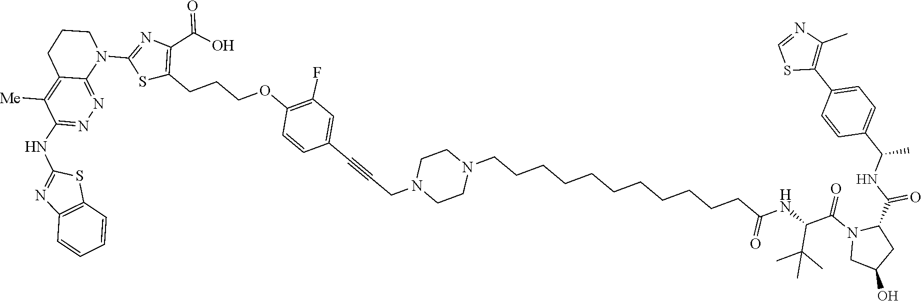 [SMILES: Cc1ncsc1-c1ccc([C@H](C)NC(=O)[C@@H]2C[C@@H](O)CN2C(=O)[C@@H](NC(=O)CCCCCCCCCCCN2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(12-(((S)-1-((2S,4R)-4-hydroxy- |
| | 2-(((S)-1-(4-(4-methylthiazol-5-yl)phenyl)ethyl)carbamoyl)pyrrolidin-1-yl)- |
| | 3,3-dimethyl-1-oxobutan-2-yl)amino)-12-oxododecyl)piperazin-1-yl)prop-1- |
| | yn-1-yl)phenoxy)propyl)thiazole-4-carboxylic acid |
| 52 | 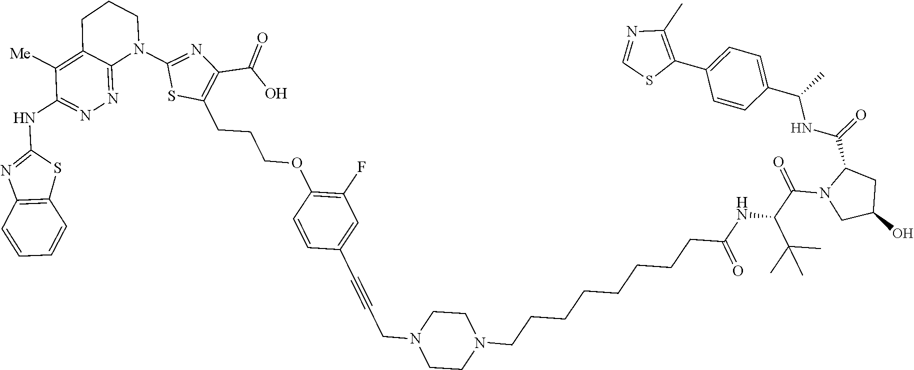 [SMILES: Cc1ncsc1-c1ccc([C@H](C)NC(=O)[C@@H]2C[C@@H](O)CN2C(=O)[C@@H](NC(=O)CCCCCCCCN2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(9-(((S)-1-((2S,4R)-4-hydroxy-2- |
| | (((S)-1-(4-(4-methylthiazol-5-yl)phenyl)ethyl)carbamoyl)pyrrolidin-1-yl)-3,3- |
| | dimethyl-1-oxobutan-2-yl)amino)-9-oxononyl)piperazin-1-yl)prop-1-yn-1- |
| | yl)phenoxy)propyl)thiazole-4-carboxylic acid |
| 53 | 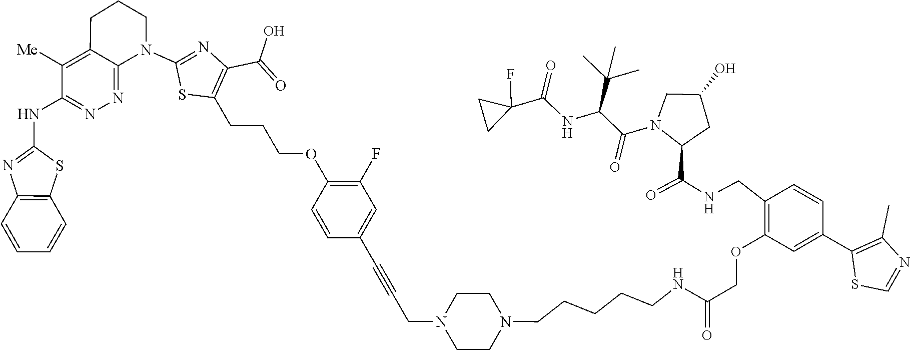 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCCN2CCN(CC#Cc3ccc(OCCCc4sc(N5CCCc6c5nnc(Nc5nc7ccccc7s5)c6C)nc4C(=O)O)c(F)c3)CC2)c1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(5-(2-(2-(((2S,4R)-1-((S)-2-(1- |
| | fluorocyclo propane-1-carboxamido)-3,3-dimethylbutanoyl)-4- |
| | hydroxypyrrolidine-2-carboxamido)methyl)-5-(4-methylthiazol-5- |
| | yl)phenoxy)acetamido)pentyl)piperazin-1-yl)prop-1-yn-1- |
| | yl)phenoxy)propyl)thiazole-4-carboxylic acid |
| 54 | 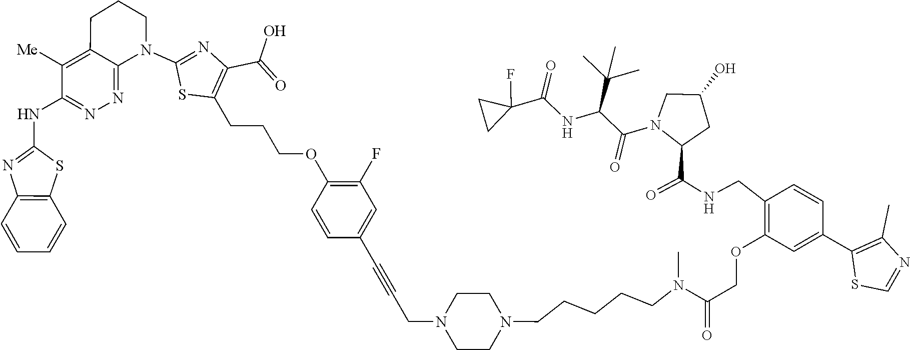 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)N(C)CCCCCN2CCN(CC#Cc3ccc(OCCCc4sc(N5CCCc6c5nnc(Nc5nc7ccccc7s5)c6C)nc4C(=O)O)c(F)c3)CC2)c1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(5-(2-(2-(((2S,4R)-1-((S)-2-(1- |
| | fluorocyclo propane-1-carboxamido)-3,3-dimethylbutanoyl)-4- |
| | hydroxypyrrolidine-2-carboxamido)methyl)-5-(4-methylthiazol-5- |
| | yl)phenoxy)-N-methylacetamido)pentyl)piperazin-1-yl)prop-1-yn-1- |
| | yl)phenoxy)propyl)thiazole-4-carboxylic acid |
| 55 | 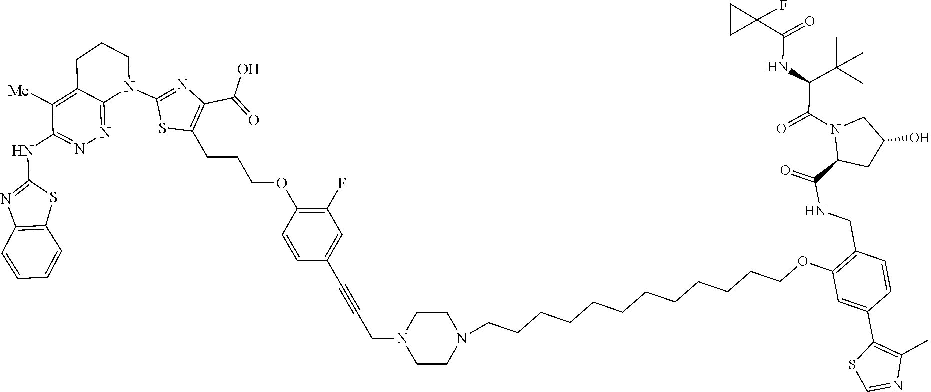 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCCCN2CCN(CC#Cc3ccc(OCCCc4sc(N5CCCc6c5nnc(Nc5nc7ccccc7s5)c6C)nc4C(=O)O)c(F)c3)CC2)c1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(12-(2-(((2S,4R)-1-((S)-2-(1- |
| | fluorocyclo propane-1-carboxamido)-3,3-dimethylbutanoyl)-4- |
| | hydroxypyrrolidine-2-carboxamido methyl)-5-(4-methylthiazol-5- |
| | yl)phenoxy)dodecyl)piperazin-1-yl)prop-1-yn-1-yl)phenoxy)propyl)thiazole- |
| | 4-carboxylic acid |
| 56 | 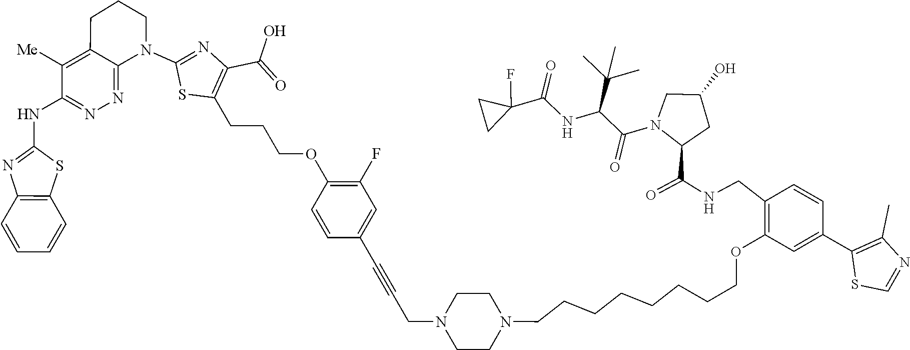 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCN2CCN(CC#Cc3ccc(OCCCc4sc(N5CCCc6c5nnc(Nc5nc7ccccc7s5)c6C)nc4C(=O)O)c(F)c3)CC2)c1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(8-(2-(((2S,4R)-1-((S)-2-(1- |
| | fluorocyclo propane-1-carboxamido)-3,3-dimethylbutanoyl)-4- |
| | hydroxypyrrolidine-2-carboxamido)methyl)-5-(4-methylthiazol-5- |
| | yl)phenoxy)octyl)piperazin-1-yl)prop-1-yn-1-yl)phenoxy)propyl)thiazole-4- |
| | carboxylic acid |
| 57 | 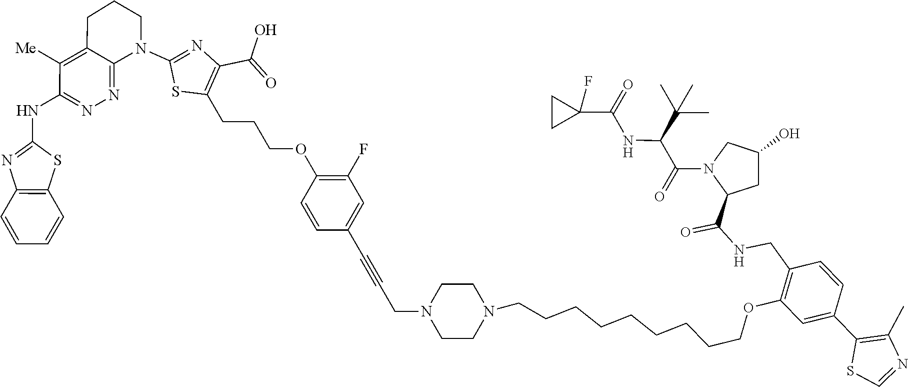 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCN2CCN(CC#Cc3ccc(OCCCc4sc(N5CCCc6c5nnc(Nc5nc7ccccc7s5)c6C)nc4C(=O)O)c(F)c3)CC2)c1] |
| | 2-(3-(benzo[d]thiazol-2-ylamino)-4-methyl-6,7-dihydropyrido [2,3- |
| | c]pyridazin-8(5H)-yl)-5-(3-(2-fluoro-4-(3-(4-(9-(2-(((2S,4R)-1-((S)-2-(1- |
| | fluorocyclo propane-1-carboxamido)-3,3-dimethylbutanoyl)-4- |
| | hydroxypyrrolidine-2-carboxamido)methyl)-5-(4-methylthiazol-5- |
| | yl)phenoxy)nonyl)piperazin-1-yl)prop-1-yn-1-yl)phenoxy)propyl)thiazole-4- |
| | carboxylic acid |
| 58 | 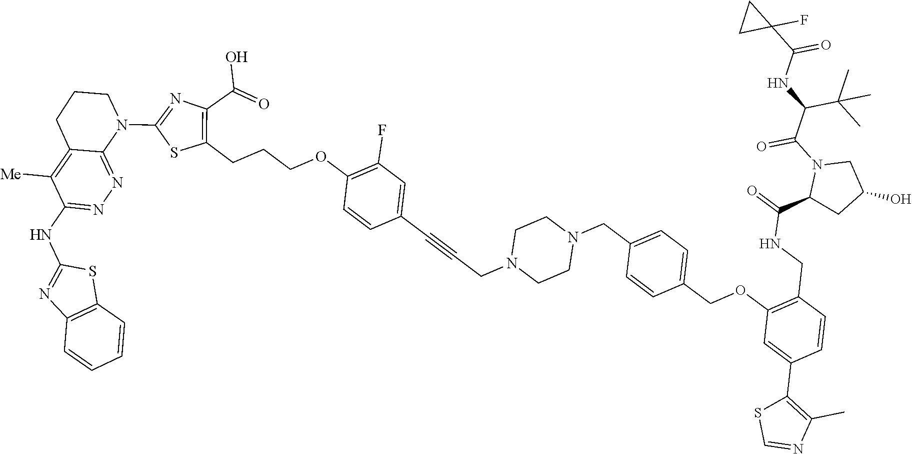 [SMILES: Cc1ncsc1-c1ccc(CNC(=O)[C@@H]2C[C@@H](O)CN2C(=O)[C@@H](NC(=O)C2(F)CC2)C(C)(C)C)c(OCc2ccc(CN3CCN(CC#Cc4ccc(OCCCc5sc(N6CCCc7c6nnc(Nc6nc8ccccc8s6)c7C)nc5C(=O)O)c(F)c4)CC3)cc2)c1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(4-((2-(((2S,4R)-1-((S)-2-(1- |
| | fluorocyclopropane-1-carboxamido)-3,3-dimethylbutanoyl)-4- |
| | hydroxypyrrolidine-2-carboxamido)methyl)-5-(4-methylthiazol-5- |
| | yl)phenoxy)methyl)benzyl)piperazin-1-yl)prop-1-yn-1- |
| | yl)phenoxy)propyl)thiazole-4-carboxylic acid |
| 59 | 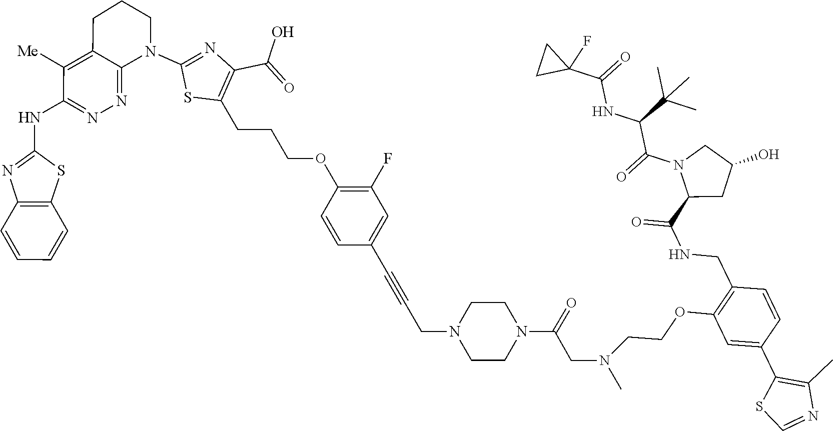 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN(C)CC(=O)N2CCN(CC#Cc3ccc(OCCCc4sc(N5CCCc6c5nnc(Nc5nc7ccccc7s5)c6C)nc4C(=O)O)c(F)c3)CC2)c1] |
| | 2-(3-(benzo[d]thiazol-2-ylamino)-4-methyl-6,7-dihydropyrido[2,3- |
| | c]pyridazin-8(5H)-yl)-5-(3-(2-fluoro-4-(3-(4-(N-(2-(2-(((2S,4R)-1-((S)-2-(1- |
| | fluorocyclo propane-1-carboxamido)-3,3-dimethylbutanoyl)-4- |
| | hydroxypyrrolidine-2-carboxamido)methyl)-5-(4-methylthiazol-5- |
| | yl)phenoxy)ethyl)-N-methylglycyl)piperazin-1-yl)prop-1-yn-1- |
| | yl)phenoxy)propyl)thiazole-4-carboxylic acid |
| 60 | 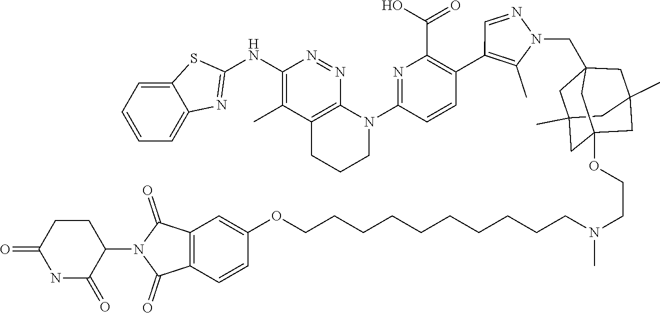 [SMILES: Cc1c(Nc2nc3ccccc3s2)nnc2c1CCCN2c1ccc(-c2cnn(CC34CC5(C)CC(C)(C3)CC(OCCN(C)CCCCCCCCCCOc3ccc6c(c3)C(=O)N(C3CCC(=O)NC3=O)C6=O)(C5)C4)c2C)c(C(=O)O)n1] |
| | 6-[3-(1,3-benzothiazol-2-ylamino)-4-methyl-6,7-dihydro-5H-pyrido[2,3- |
| | c]pyridazin-8-yl]-3-[1-[3-[2-[10-[2-(2,6-dioxo-3-piperidyl)-1,3-dioxo- |
| | isoindolin-5-yl]oxydecyl-methyl-amino]ethoxy]-5,7-dimethyl-1- |
| | adamantyl]methyl]-5-methyl-pyrazol-4-yl]pyridine-2-carboxylic acid |
| 61 | 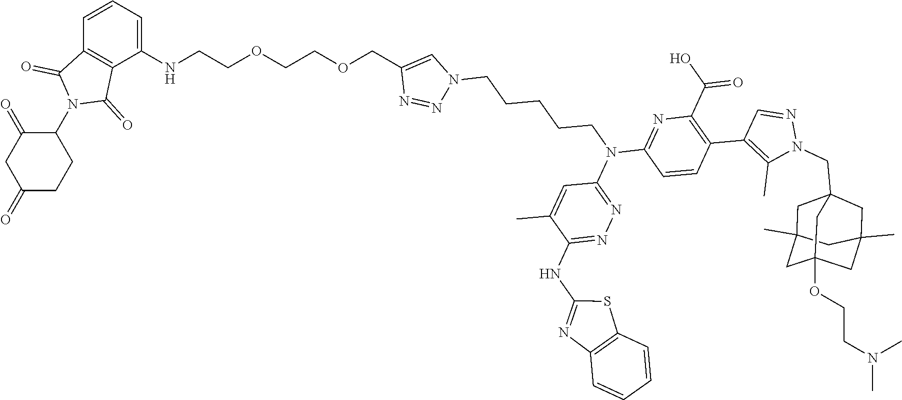 [SMILES: Cc1cc(N(CCCCCn2cc(COCCOCCNc3cccc4c3C(=O)N(C3CCC(=O)CC3=O)C4=O)nn2)c2ccc(-c3cnn(CC45CC6(C)CC(C)(C4)CC(OCCN(C)C)(C6)C5)c3C)c(C(=O)O)n2)nnc1Nc1nc2ccccc2s1] |
| | 6-[[6-(1,3-benzothiazol-2-ylamino)-5-methyl-pyridazin-3-yl]-[5-[4-[2-[2-[[2- |
| | (2,6-dioxo-3-piperidyl)-1,3-dioxo-isoindolin-4- |
| | yl]amino]ethoxy]ethoxymethyl]triazol-1-yl]pentyl]amino]-3-[1-[3-[2- |
| | (dimethylamino)ethoxy]-5,7-dimethyl-1-adamantyl]methyl]-5-methyl- |
| | pyrazol-4-yl]pyridine-2-carboxylic acid |
| 62 | 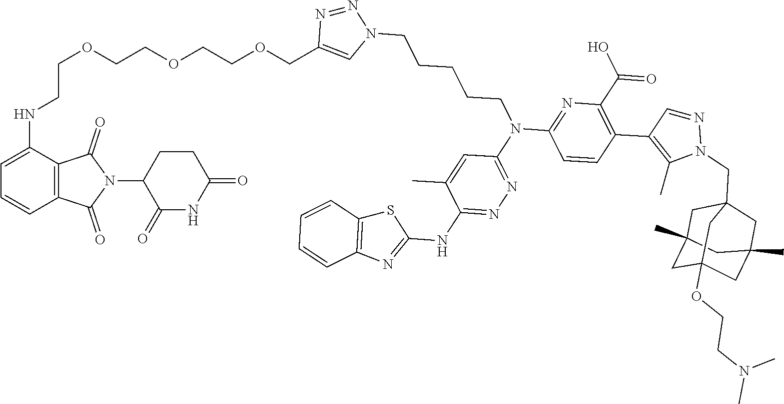 [SMILES: Cc1cc(N(CCCCCn2cc(COCCOCCOCCNc3cccc4c3C(=O)N(C3CCC(=O)NC3=O)C4=O)nn2)c2ccc(-c3cnn(CC45CC6(OCCN(C)C)C[C@](C)(C4)C[C@](C)(C5)C6)c3C)c(C(=O)O)n2)nnc1Nc1nc2ccccc2s1] |
| | 6-[[6-(1,3-benzothiazol-2-ylamino)-5-methyl-pyridazin-3-yl]-[5-[4-[2-[2-[2-[[2- |
| | (2,6-dioxo-3-piperidyl)-1,3-dioxo-isoindolin-4- |
| | yl]amino]ethoxy]ethoxy]ethoxymethyl]triazol-1-yl]pentyl]amino]-3-[1-[3-[2- |
| | (dimethylamino)ethoxy]-5,7-dimethyl-1-adamantyl]methyl]-5-methyl- |
| | pyrazol-4-yl]pyridine-2-carboxylic acid |
| 63 | 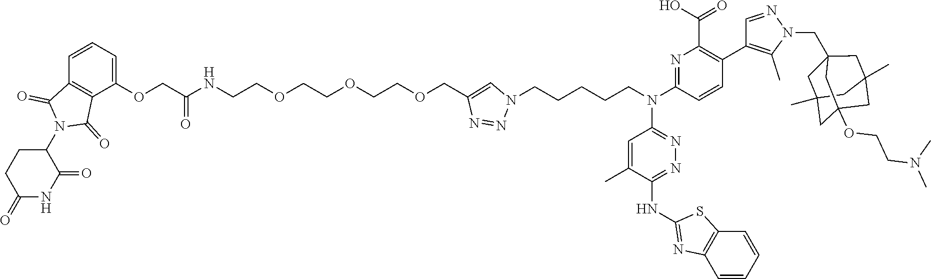 [SMILES: Cc1cc(N(CCCCCn2cc(COCCOCCOCCNC(=O)COc3cccc4c3C(=O)N(C3CCC(=O)NC3=O)C4=O)nn2)c2ccc(-c3cnn(CC45CC6(C)CC(C)(C4)CC(OCCN(C)C)(C6)C5)c3C)c(C(=O)O)n2)nnc1Nc1nc2ccccc2s1] |
| | 6-[[6-(1,3-benzothiazol-2-ylamino)-5-methyl-pyridazin-3-yl]-[5-[4-[2-[2-[2-[[2- |
| | [2-(2,6-dioxo-3-piperidyl)-1,3-dioxo-isoindolin-4- |
| | yl]oxyacetyl]amino]ethoxy]ethoxy]ethoxymethyl]triazol-1-yl]pentyl]amino]-3- |
| | [1-[3-[2-(dimethylamino)ethoxy]-5,7-dimethyl-1-adamantyl]methyl]-5- |
| | methyl-pyrazol-4-yllpyridine-2-carboxylic acid |
| 64 | 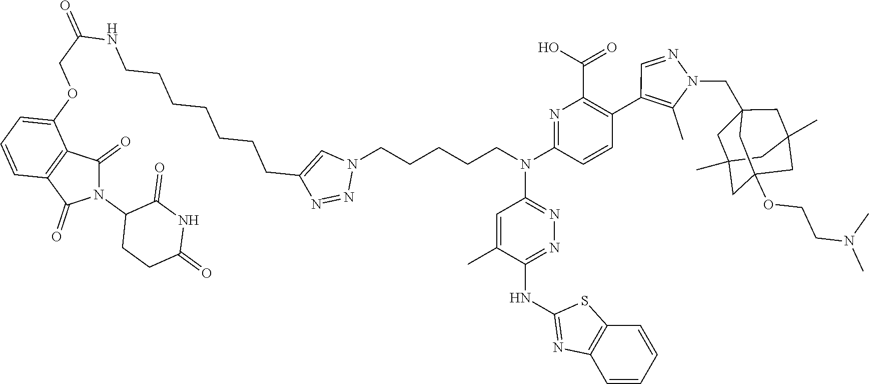 [SMILES: Cc1cc(N(CCCCCn2cc(CCCCCCCNC(=O)COc3cccc4c3C(=O)N(C3CCC(=O)NC3=O)C4=O)nn2)c2ccc(-c3cnn(CC45CC6(C)CC(C)(C4)CC(OCCN(C)C)(C6)C5)c3C)c(C(=O)O)n2)nnc1Nc1nc2ccccc2s1] |
| | 6-[[6-(1,3-benzothiazol-2-ylamino)-5-methyl-pyridazin-3-yl]-[5-[4-[7-[[2-[2- |
| | (2,6-dioxo-3-piperidyl)-1,3-dioxo-isoindolin-4- |
| | yl]oxyacetyl]amino]heptyl]triazol-1-yl]pentyl]amino]-3-[1-[[3-[2- |
| | (dimethylamino)ethoxy]-5,7-dimethyl-1-adamantyl]methyl]-5-methyl- |
| | pyrazol-4-yl]pyridine-2-carboxylic acid |
| 65 | 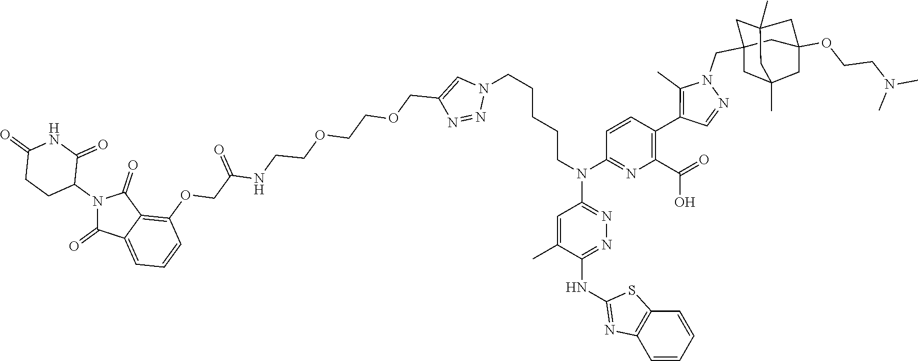 [SMILES: Cc1cc(N(CCCCCn2cc(COCCOCCNC(=O)COc3cccc4c3C(=O)N(C3CCC(=O)NC3=O)C4=O)nn2)c2ccc(-c3cnn(CC45CC6(C)CC(C)(C4)CC(OCCN(C)C)(C6)C5)c3C)c(C(=O)O)n2)nnc1Nc1nc2ccccc2s1] |
| | 6-[[6-(1,3-benzothiazol-2-ylamino)-5-methyl-pyridazin-3-yl]-[5-[4-[2-[2-[[2-[2- |
| | (2,6-dioxo-3-piperidyl)-1,3-dioxo-isoindolin-4- |
| | yl]oxyacetyl]amino]ethoxy]ethoxymethyl]triazol-1-yl]pentyl]amino]-3-[1-[[3- |
| | [2-(dimethylamino)ethoxy]-5,7-dimethyl-1-adamantyl]methyl]-5-methyl- |
| | pyrazol-4-yllpyridine-2-carboxylic acid |
| 66 | 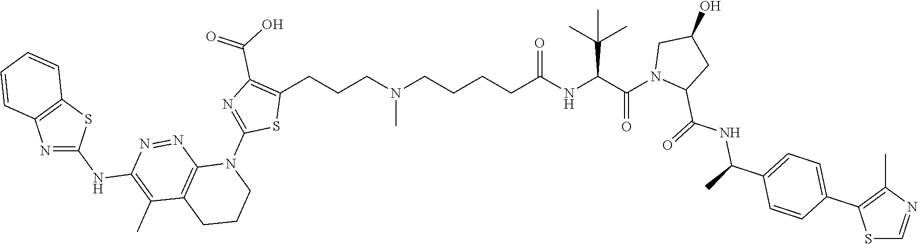 [SMILES: Cc1ncsc1-c1ccc([C@@H](C)NC(=O)C2C[C@H](O)CN2C(=O)[C@@H](NC(=O)CCCCN(C)CCCc2sc(N3CCCc4c3nnc(Nc3nc5ccccc5s3)c4C)nc2C(=O)O)C(C)(C)C)cc1] |
| | 2-[3-(1,3-benzothiazol-2-ylamino)-4-methyl-6,7-dihydro-5H-pyrido[2,3- |
| | c]pyridazin-8-yl]-5-[3-[[5-[[(1S)-1-[(2S,4R)-4-hydroxy-2-[[(1S)-1-[4-(4- |
| | methylthiazol-5-yl)phenyl]ethyl]carbamoyl]pyrrolidine-1-carbonyl]-2,2- |
| | dimethyl-propyl]amino]-5-oxo-pentyl]-methyl-amino]propyl]thiazole-4- |
| | carboxylic acid |
| 67 | 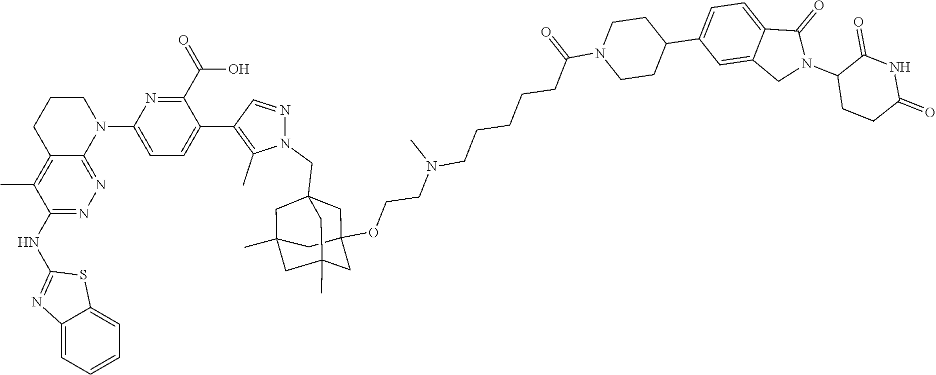 [SMILES: Cc1c(Nc2nc3ccccc3s2)nnc2c1CCCN2c1ccc(-c2cnn(CC34CC5(C)CC(C)(C3)CC(OCCN(C)CCCCCC(=O)N3CCC(c6ccc7c(c6)CN(C6CCC(=O)NC6=O)C7=O)CC3)(C5)C4)c2C)c(C(=O)O)n1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[[6-[4-[2-(2,6-dioxo-3-piperidyl)-1-oxo-isoindolin- |
| | 5-yl]-1-piperidyl]-6-oxo-hexyl]-methyl-amino]ethoxy]-5,7-dimethyl-1- |
| | adamantyl]methyl]-5-methyl-pyrazol-4-yl]pyridine-2-carboxylic acid |
| 68 | 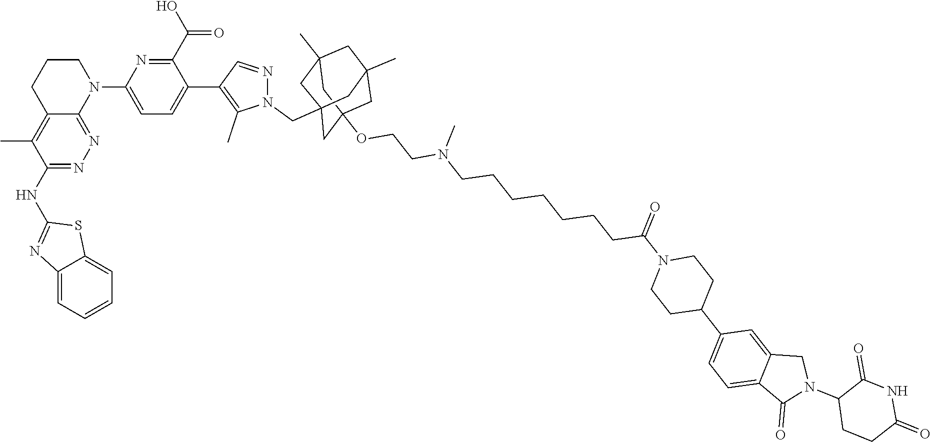 [SMILES: Cc1c(Nc2nc3ccccc3s2)nnc2c1CCCN2c1ccc(-c2cnn(CC34CC5(C)CC(C)(C3)CC(OCCN(C)CCCCCCCC(=O)N3CCC(c6ccc7c(c6)CN(C6CCC(=O)NC6=O)C7=O)CC3)(C5)C4)c2C)c(C(=O)O)n1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[[6-[4-[2-(2,6-dioxo-3-piperidyl)-1-oxo-isoindolin- |
| | 5-yl]-1-piperidyl]-6-oxo-hexyl]-methyl-amino]ethoxy]-5,7-dimethyl-1- |
| | adamantyl]methyl]-5-methyl-pyrazol-4-yl]pyridine-2-carboxylic acid |
| 69 | 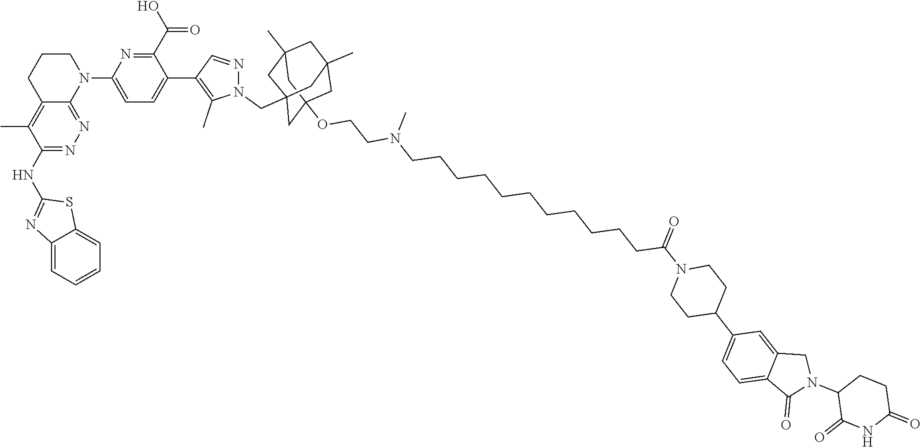 [SMILES: Cc1c(Nc2nc3ccccc3s2)nnc2c1CCCN2c1ccc(-c2cnn(CC34CC5(C)CC(C)(C3)CC(OCCN(C)CCCCCCCCCCCC(=O)N3CCC(c6ccc7c(c6)CN(C6CCC(=O)NC6=O)C7=O)CC3)(C5)C4)c2C)c(C(=O)O)n1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[[12-[4-[2-(2,6-dioxo-3-piperidyl)-1-oxo- |
| | isoindolin-5-yl]-1-piperidyl]-12-oxo-dodecyl]-methyl-amino]ethoxy]-5,7- |
| | dimethyl-1-adamantyl]methyl]-5-methyl-pyrazol-4-yl]pyridine-2-carboxylic |
| | acid |
| 70 | 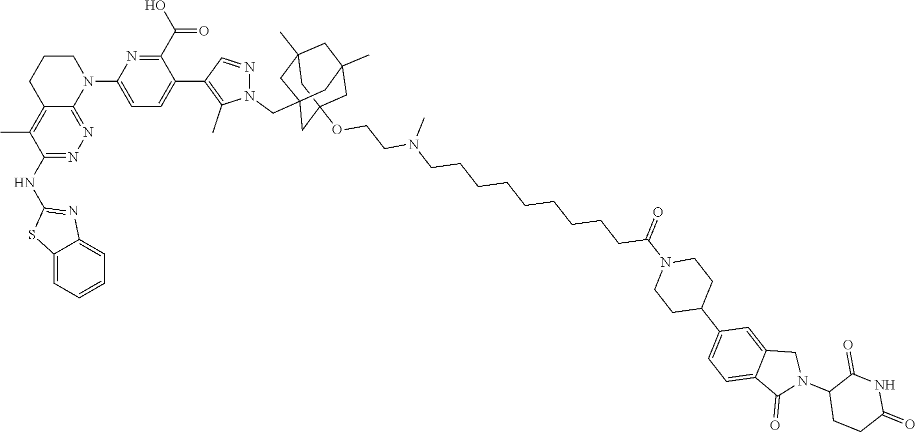 [SMILES: Cc1c(Nc2nc3ccccc3s2)nnc2c1CCCN2c1ccc(-c2cnn(CC34CC5(C)CC(C)(C3)CC(OCCN(C)CCCCCCCCCC(=O)N3CCC(c6ccc7c(c6)CN(C6CCC(=O)NC6=O)C7=O)CC3)(C5)C4)c2C)c(C(=O)O)n1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[[10-[4-[2-(2,6-dioxo-3-piperidyl)-1-oxo- |
| | isoindolin-5-yl]-1-piperidyl]-10-oxo-decyl]-methyl-amino]ethoxy]-5,7- |
| | dimethyl-1-adamantyl]methyl]-5-methyl-pyrazol-4-yl]pyridine-2-carboxylic |
| | acid |
| 71 | 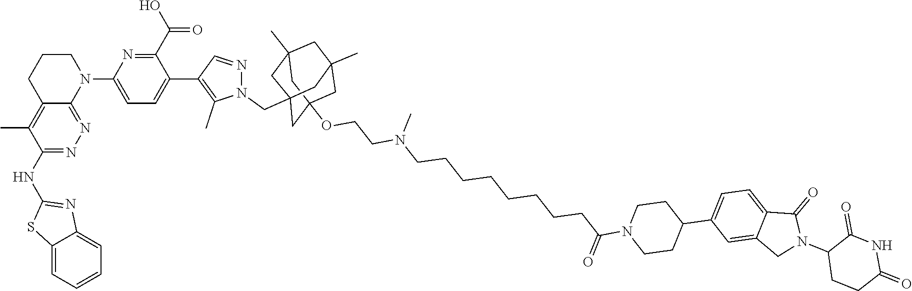 [SMILES: Cc1c(Nc2nc3ccccc3s2)nnc2c1CCCN2c1ccc(-c2cnn(CC34CC5(C)CC(C)(C3)CC(OCCN(C)CCCCCCCCC(=O)N3CCC(c6ccc7c(c6)CN(C6CCC(=O)NC6=O)C7=O)CC3)(C5)C4)c2C)c(C(=O)O)n1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[[9-[4-[2-(2,6-dioxo-3-piperidyl)-1-oxo-isoindolin- |
| | 5-yl]-1-piperidyl]-9-oxo-nonyl]-methyl-amino]ethoxy]-5,7-dimethyl-1- |
| | adamantyl]methyl]-5-methyl-pyrazol-4-yl]pyridine-2-carboxylic acid |
| 72 | 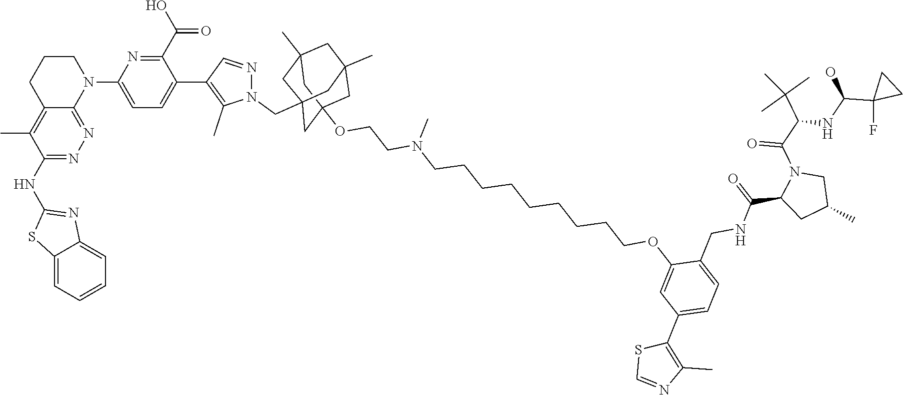 [SMILES: Cc1ncsc1-c1ccc(CNC(=O)[C@@H]2C[C@@H](C)CN2C(=O)[C@@H](N[C@@H](O)C2(F)CC2)C(C)(C)C)c(OCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)c1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[9-[2-[[[(2S,4R)-1-[(2S)-2-[(1- |
| | fluorocyclopropanecarbonyl)amino]-3,3-dimethyl-butanoyl]-4-hydroxy- |
| | pyrrolidine-2-carbonyl]amino]methyl]-5-(4-methylthiazol-5- |
| | yl)phenoxy]nonyl-methyl-amino]ethoxy]-5,7-dimethyl-1-adamantyl]methyl]- |
| | 5-methyl-pyrazol-4-yl]pyridine-2-carboxylic acid |
| 73 | 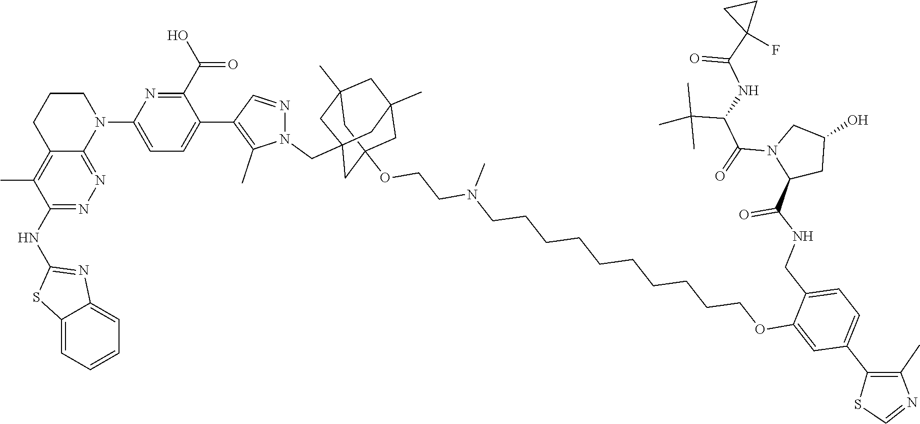 |
| | 6-[3-(1,3-benzothiazol-2-ylamino)-4-methyl-6,7-dihydro-5H-pyrido[2,3- |
| | c]pyridazin-8-yl]-3-[1-[[3-[2-[10-[2-[[[(2S,4R)-1-[(2S)-2-[(1- |
| | fluorocyclopropanecarbonyl)amino]-3,3-dimethyl-butanoyl]-4-hydroxy- |
| | pyrrolidine-2-carbonyl]amino]methyl]-5-(4-methylthiazol-5- |
| | yl)phenoxy]decyl-methyl-amino]ethoxy]-5,7-dimethyl-1-adamantyl]methyl]- |
| | 5-methyl-pyrazol-4-yl]pyridine-2-carboxylic acid |
| 74 | 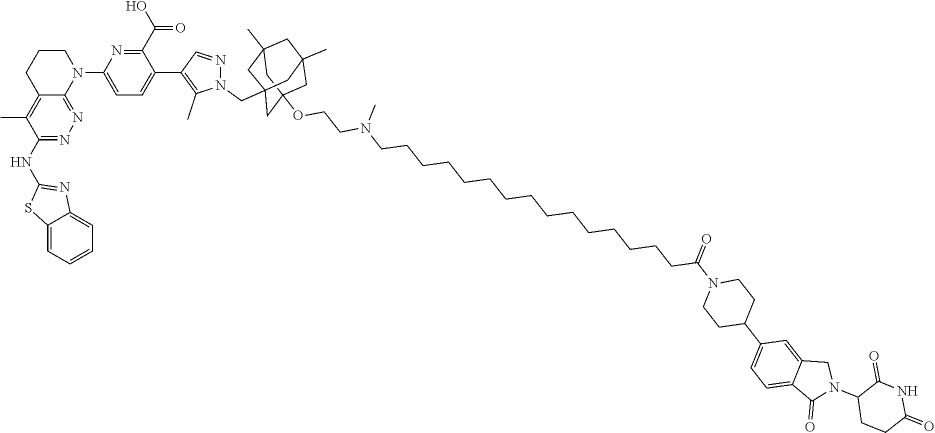 [SMILES: Cc1c(Nc2nc3ccccc3s2)nnc2c1CCCN2c1ccc(-c2cnn(CC34CC5(C)CC(C)(C3)CC(OCCN(C)CCCCCCCCCCCCCCCC(=O)N3CCC(c6ccc7c(c6)CN(C6CCC(=O)NC6=O)C7=O)CC3)(C5)C4)c2C)c(C(=O)O)n1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[[16-[4-[2-(2,6-dioxo-3-piperidyl)-1-oxo- |
| | isoindolin-5-yl]-1-piperidyl]-16-oxo-hexadecyl]-methyl-amino]ethoxy]-5,7- |
| | dimethyl-1-adamantyl]methyl]-5-methyl-pyrazol-4-yl]pyridine-2-carboxylic |
| | acid |
| 75 | 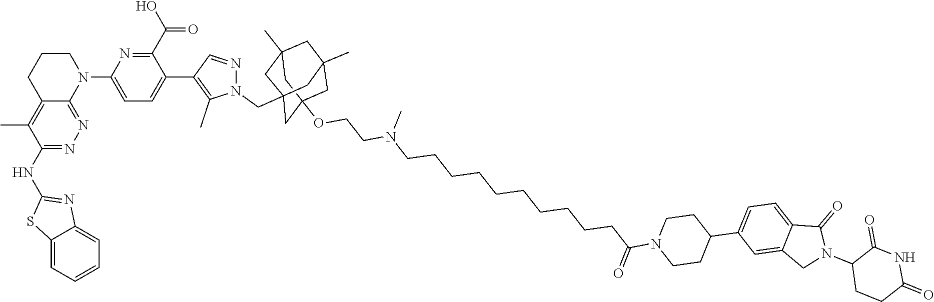 [SMILES: Cc1c(Nc2nc3ccccc3s2)nnc2c1CCCN2c1ccc(-c2cnn(CC34CC5(C)CC(C)(C3)CC(OCCN(C)CCCCCCCCCCC(=O)N3CCC(c6ccc7c(c6)CN(C6CCC(=O)NC6=O)C7=O)CC3)(C5)C4)c2C)c(C(=O)O)n1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[[11-[4-[2-(2,6-dioxo-3-piperidyl)-1-oxo- |
| | isoindolin-5-yl]-1-piperidyl]-11-oxo-undecyl]-methyl-amino]ethoxy]-5,7- |
| | dimethyl-1-adamantyl]methyl]-5-methyl-pyrazol-4-yl]pyridine-2-carboxylic |
| | acid |
| 76 | 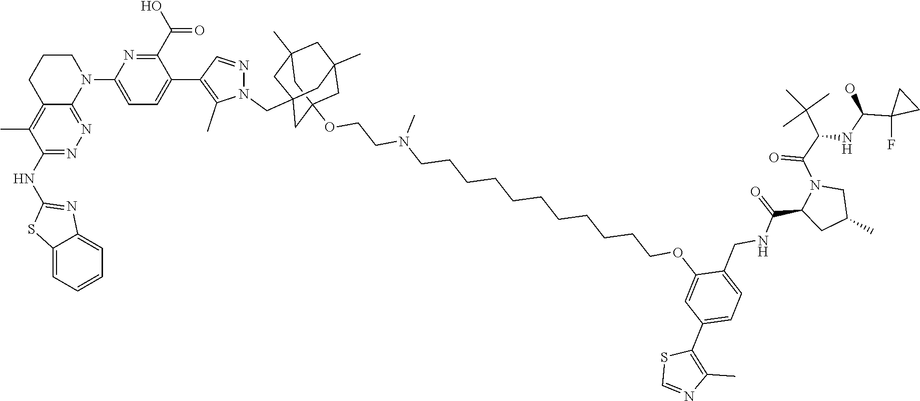 [SMILES: Cc1ncsc1-c1ccc(CNC(=O)[C@@H]2C[C@@H](C)CN2C(=O)[C@@H](N[C@@H](O)C2(F)CC2)C(C)(C)C)c(OCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)c1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[11-[2-[[[(2S,4R)-1-[(2S)-2-[(1- |
| | fluorocyclopropanecarbonyl)amino]-3,3-dimethyl-butanoyl]-4-hydroxy- |
| | pyrrolidine-2-carbonyl]amino]methyl]-5-(4-methylthiazol-5- |
| | yl)phenoxy]undecyl-methyl-amino]ethoxy]-5,7-dimethyl-1- |
| | adamantyl]methyl]-5-methyl-pyrazol-4-yl]pyridine-2-carboxylic acid |
| 77 | 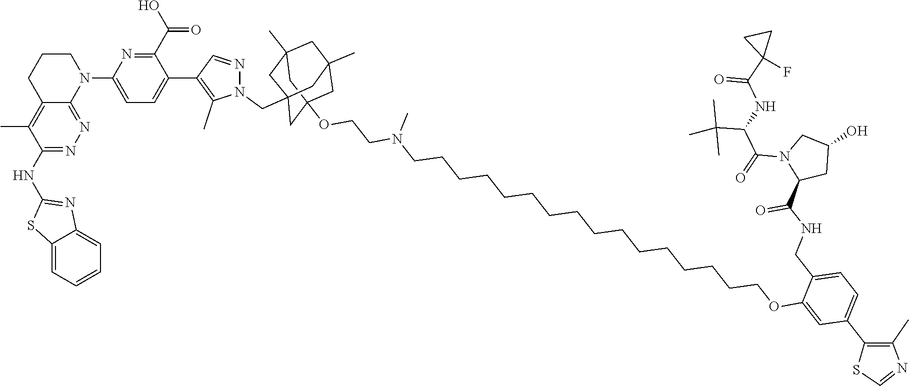 |
| | 6-[3-(1,3-benzothiazol-2-ylamino)-4-methyl-6,7-dihydro-5H-pyrido[2,3- |
| | c]pyridazin-8-yl]-3-[1-[[3-[2-[16-[2-[[[(2S,4R)-1-[(2S)-2-[(1- |
| | fluorocyclopropanecarbonyl)amino]-3,3-dimethyl-butanoyl]-4-hydroxy- |
| | pyrrolidine-2-carbonyl]amino]methyl]-5-(4-methylthiazol-5- |
| | yl)phenoxy]hexadecyl-methyl-amino]ethoxy]-5,7-dimethyl-1- |
| | adamantyl]methyl]-5-methyl-pyrazol-4-yl]pyridine-2-carboxylic acid |
| 78 | 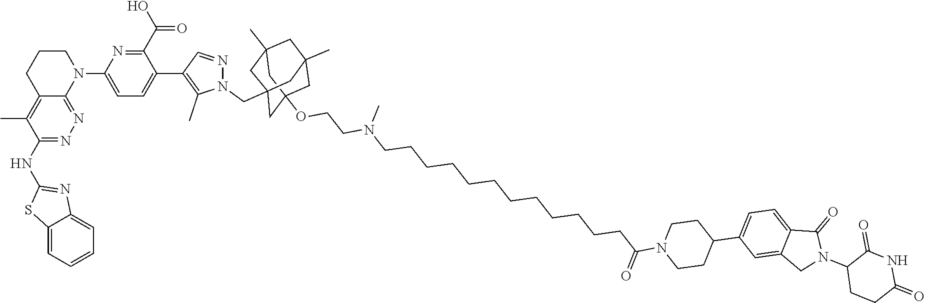 [SMILES: Cc1c(Nc2nc3ccccc3s2)nnc2c1CCCN2c1ccc(-c2cnn(CC34CC5(C)CC(C)(C3)CC(OCCN(C)CCCCCCCCCCCCC(=O)N3CCC(c6ccc7c(c6)CN(C6CCC(=O)NC6=O)C7=O)CC3)(C5)C4)c2C)c(C(=O)O)n1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[[13-[4-[2-(2,6-dioxo-3-piperidyl)-1-oxo- |
| | isoindolin-5-yl]-1-piperidyl]-13-oxo-tridecyl]-methyl-amino]ethoxy]-5,7- |
| | dimethyl-1-adamantyl]methyl]-5-methyl-pyrazol-4-yl]pyridine-2-carboxylic |
| | acid |
| 79 | 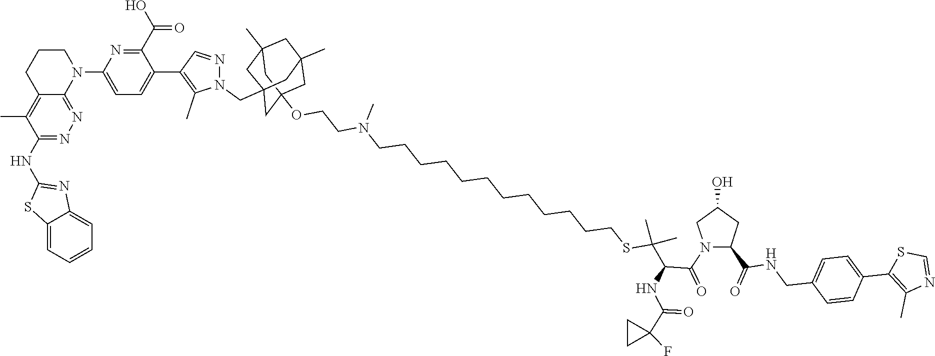 [SMILES: Cc1ncsc1-c1ccc(CNC(=O)[C@@H]2C[C@@H](O)CN2C(=O)[C@@H](NC(=O)C2(F)CC2)C(C)(C)SCCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)cc1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[12-[(2R)-2-[(1- |
| | fluorocyclopropanecarbonyl)amino]-3-[(2S,4R)-4-hydroxy-2-[[4-(4- |
| | methylthiazol-5-yl)phenyl]methylcarbamoyl]pyrrolidin-1-yl]-1,1-dimethyl-3- |
| | oxo-propyl]sulfanyldodecyl-methyl-amino]ethoxy]-5,7-dimethyl-1- |
| | adamantyl]methyl]-5-methyl-pyrazol-4-yl]pyridine-2-carboxylic acid |
| 80 | 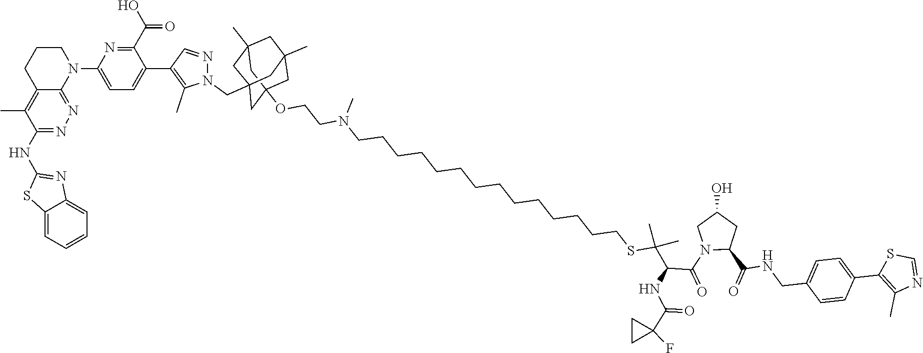 [SMILES: Cc1ncsc1-c1ccc(CNC(=O)[C@@H]2C[C@@H](O)CN2C(=O)[C@@H](NC(=O)C2(F)CC2)C(C)(C)SCCCCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)cc1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[14-[(2R)-2-[(1- |
| | fluorocyclopropanecarbonyl)amino]-3-[(2S,4R)-4-hydroxy-2-[[4-(4- |
| | methylthiazol-5-yl)phenyl]methylcarbamoyl]pyrrolidin-1-yl]-1,1-dimethyl-3- |
| | oxo-propyl]sulfanyltetradecyl-methyl-amino]ethoxy]-5,7-dimethyl-1- |
| | adamantyl]methyl]-5-methyl-pyrazol-4-yl]pyridine-2-carboxylic acid |
| 81 | 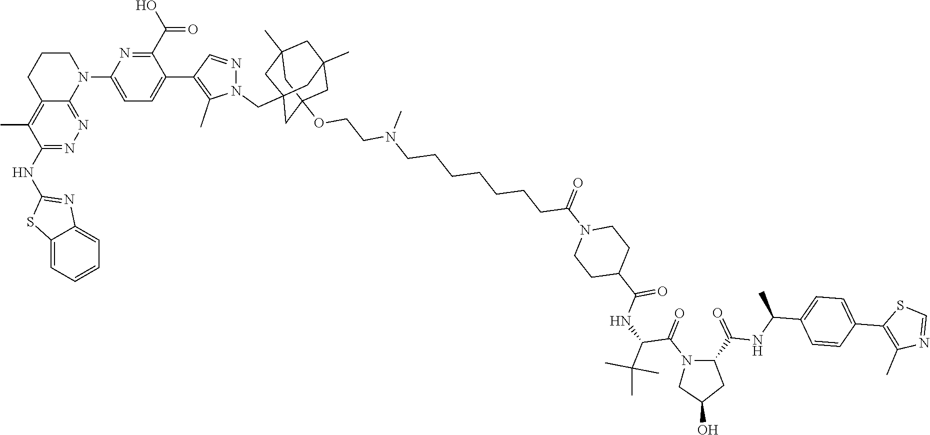 [SMILES: Cc1ncsc1-c1ccc([C@H](C)NC(=O)[C@@H]2C[C@@H](O)CN2C(=O)[C@@H](NC(=O)C2CCN(C(=O)CCCCCCCN(C)CCOC34CC5(C)CC(C)(CC(Cn6ncc(-c7ccc(N8CCCc9c8nnc(Nc8nc%10ccccc%10s8)c9C)nc7C(=O)O)c6C)(C5)C3)C4)CC2)C(C)(C)C)cc1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[[8-[4-[[(1S)-1-[(2S,4R)-4-hydroxy-2-[[(1S)-1-[4- |
| | (4-methylthiazol-5-yl)phenyl]ethyl]carbamoyl]pyrrolidine-1-carbonyl]-2,2- |
| | dimethyl-propyl]carbamoyl]-1-piperidyl]-8-oxo-octyl]-methyl-amino]ethoxy]- |
| | 5,7-dimethyl-1-adamantyl]methyl]-5-methyl-pyrazol-4-yl]pyridine-2- |
| | carboxylic acid |
| 82 | 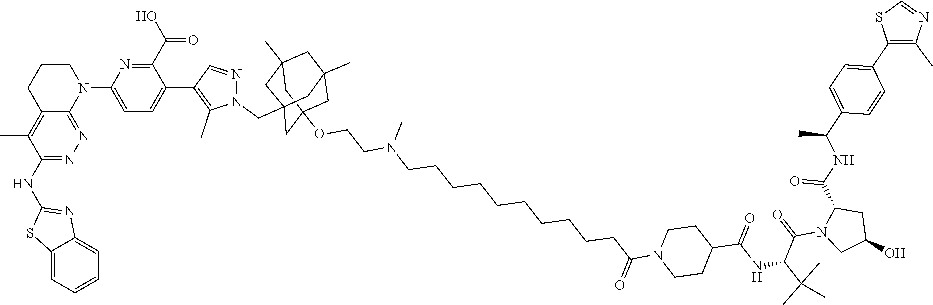 |
| | 6-[3-(1,3-benzothiazol-2-ylamino)-4-methyl-6,7-dihydro-5H-pyrido[2,3-c] |
| | pyridazin-8-yl]-3-[1-[[3-[2-[[11-[4-[[(1S)-1-[(2S,4R)-4-hydroxy-2-[[(1S)-1-[4- |
| | (4-methylthiazol-5-yl)phenyl]ethyl]carbamoyl]pyrrolidine-1-carbonyl]-2,2- |
| | dimethyl-propyl]carbamoyl]-1-piperidyl]-11-oxo-undecyl]-methyl- |
| | amino]ethoxy]-5,7-dimethyl-1-adamantyl]methyl]-5-methyl-pyrazol-4- |
| | yl]pyridine-2-carboxylic acid |
| 83 | 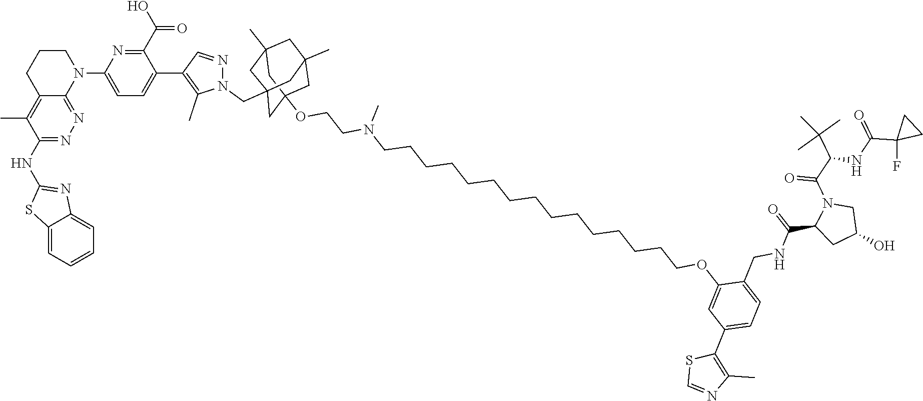 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)c1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[15-[2-[[[(2S,4R)-1-[(2S)-2-[(1- |
| | fluorocyclopropanecarbonyl)amino]-3,3-dimethyl-butanoyl]-4-hydroxy- |
| | pyrrolidine-2-carbonyl]amino]methyl]-5-(4-methylthiazol-5- |
| | yl)phenoxy]pentadecyl-methyl-amino]ethoxy]-5,7-dimethyl-1- |
| | adamantyl]methyl]-5-methyl-pyrazol-4-yl]pyridine-2-carboxylic acid |
| 84 | 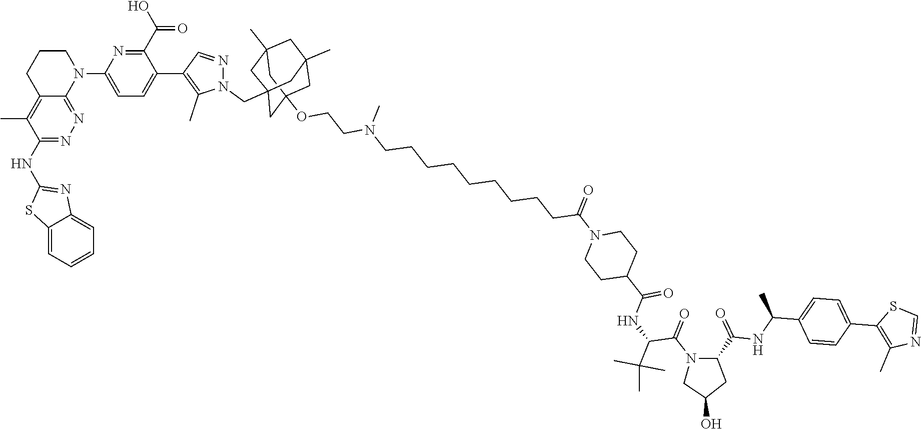 [SMILES: Cc1ncsc1-c1ccc([C@H](C)NC(=O)[C@@H]2C[C@@H](O)CN2C(=O)[C@@H](NC(=O)C2CCN(C(=O)CCCCCCCCCN(C)CCOC34CC5(C)CC(C)(CC(Cn6ncc(-c7ccc(N8CCCc9c8nnc(Nc8nc%10ccccc%10s8)c9C)nc7C(=O)O)c6C)(C5)C3)C4)CC2)C(C)(C)C)cc1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[[10-[4-[[(1S)-1-[(2S,4R)-4-hydroxy-2-[[(1S)-1-[4- |
| | (4-methylthiazol-5-yl)phenyl]ethyl]carbamoyl]pyrrolidine-1-carbonyl]-2,2- |
| | dimethyl-propyl]carbamoyl]-1-piperidyl]-10-oxo-decyl]-methyl- |
| | amino]ethoxy]-5,7-dimethyl-1-adamantyl]methyl]-5-methyl-pyrazol-4- |
| | yl]pyridine-2-carboxylic acid |
| 85 | 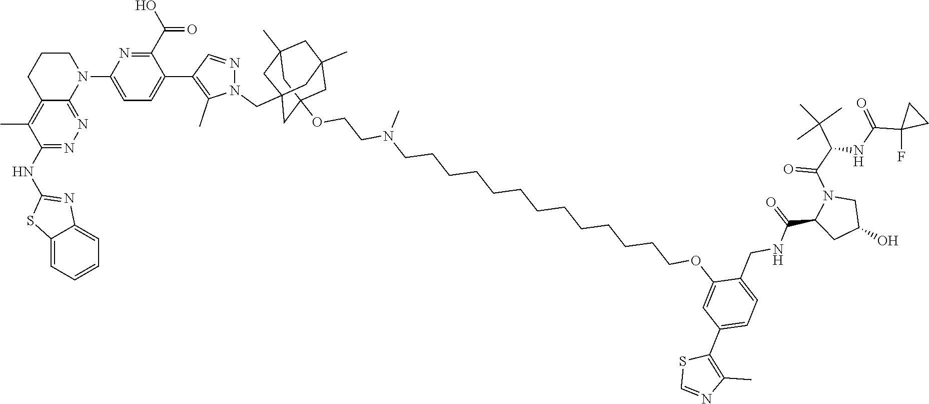 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)c1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[13-[2-[[[(2S,4R)-1-[(2S)-2-[(1- |
| | fluorocyclopropanecarbonyl)amino]-3,3-dimethyl-butanoyl]-4-hydroxy- |
| | pyrrolidine-2-carbonyl]amino]methyl]-5-(4-methylthiazol-5- |
| | yl)phenoxy]tridecyl-methyl-amino]ethoxy]-5,7-dimethyl-1- |
| | adamantyl]methyl]-5-methyl-pyrazol-4-yl]pyridine-2-carboxylic acid |
| 86 | 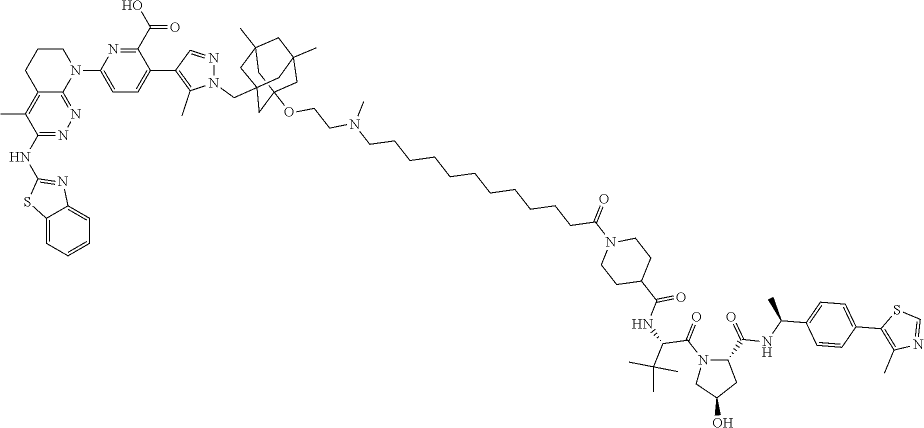 [SMILES: Cc1ncsc1-c1ccc([C@H](C)NC(=O)[C@@H]2C[C@@H](O)CN2C(=O)[C@@H](NC(=O)C2CCN(C(=O)CCCCCCCCCCCN(C)CCOC34CC5(C)CC(C)(CC(Cn6ncc(-c7ccc(N8CCCc9c8nnc(Nc8nc%10ccccc%10s8)c9C)nc7C(=O)O)c6C)(C5)C3)C4)CC2)C(C)(C)C)cc1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[13-[2-[[[(2S,4R)-1-[(2S)-2-[(1- |
| | fluorocyclopropanecarbonyl)amino]-3,3-dimethyl-butanoyl]-4-hydroxy- |
| | pyrrolidine-2-carbonyl]amino]methyl]-5-(4-methylthiazol-5- |
| | yl)phenoxy]tridecyl-methyl-amino]ethoxy]-5,7-dimethyl-1- |
| | adamantyl]methyl]-5-methyl-pyrazol-4-yl]pyridine-2-carboxylic acid |
| 87 | 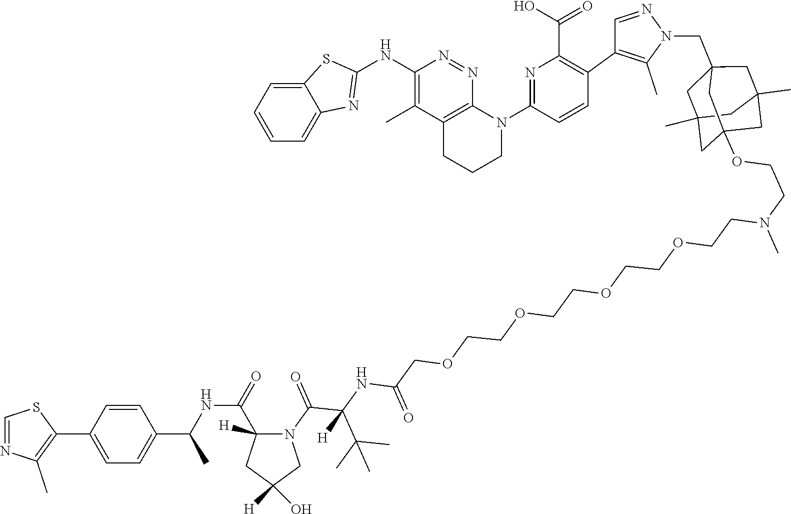 [SMILES: Cc1ncsc1-c1ccc([C@H](C)NC(=O)[C@H]2C[C@@H](O)CN2C(=O)[C@H](NC(=O)COCCOCCOCCOCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[2-[2-[2-[2-[2-[[(1S)-1-[(2S,4R)-4-hydroxy-2- |
| | [[(1S)-1-[4-(4-methylthiazol-5-yl)phenyl]ethyl]carbamoyl]pyrrolidine-1- |
| | carbonyl]-2,2-dimethyl-propyl]amino]-2-oxo- |
| | ethoxy]ethoxy]ethoxy]ethoxy]ethyl-methyl-amino]ethoxy]-5,7-dimethyl-1- |
| | adamantyl]methyl]-5-methyl-pyrazol-4-yl]pyridine-2-carboxylic acid |
| 88 | 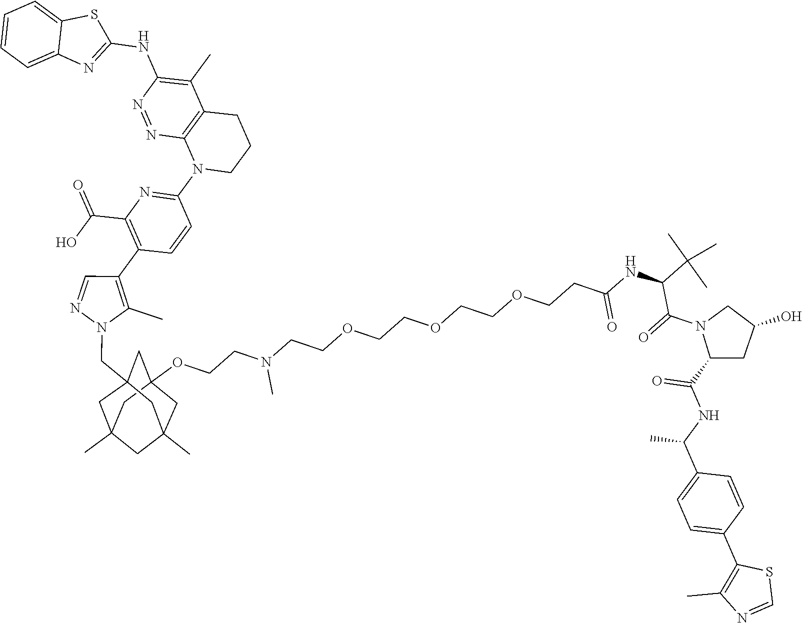 [SMILES: Cc1ncsc1-c1ccc([C@H](C)NC(=O)[C@H]2C[C@@H](O)CN2C(=O)[C@@H](NC(=O)CCOCCOCCOCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[2-[2-[2-[3-[[(1S)-1-[(2R,4R)-4-hydroxy-2-[[(1S)- |
| | 1-[4-(4-methylthiazol-5-yl)phenyl]ethyl]carbamoyl]pyrrolidine-1-carbonyl]- |
| | 2,2-dimethyl-propyl]amino]-3-oxo-propoxy]ethoxy]ethoxy]ethyl-methyl- |
| | amino]ethoxy]-5,7-dimethyl-1-adamantyl]methyl]-5-methyl-pyrazol-4- |
| | yl]pyridine-2-carboxylic acid |
| 89 | 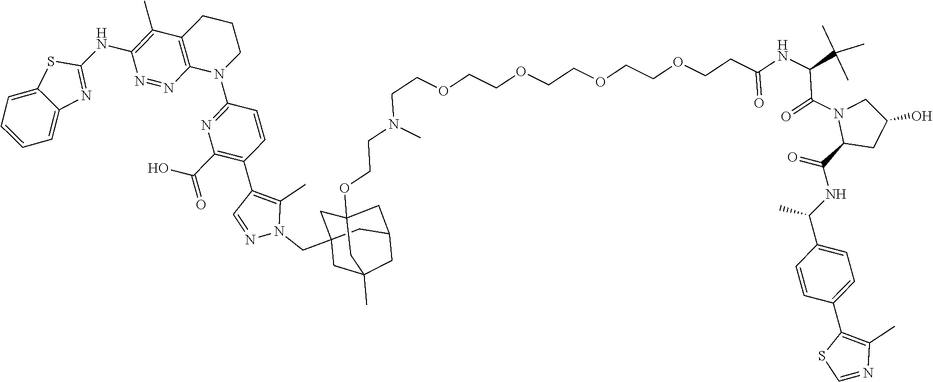 [SMILES: Cc1ncsc1-c1ccc([C@H](C)NC(=O)[C@@H]2C[C@@H](O)CN2C(=O)[C@@H](NC(=O)CCOCCOCCOCCOCCN(C)CCOC23CC4CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[2-[2-[2-[2-[3-[[(1S)-1-[(2S,4R)-4-hydroxy-2- |
| | [[(1S)-1-[4-(4-methylthiazol-5-yl)phenyl]ethyl]carbamoyl]pyrrolidine-1- |
| | carbonyl]-2,2-dimethyl-propyl]amino]-3-oxo- |
| | propoxy]ethoxy]ethoxy]ethoxy]ethyl-methyl-amino]ethoxy]-5,7-dimethyl-1- |
| | adamantyl]methyl]-5-methyl-pyrazol-4-yl]pyridine-2-carboxylic acid |
| 90 | 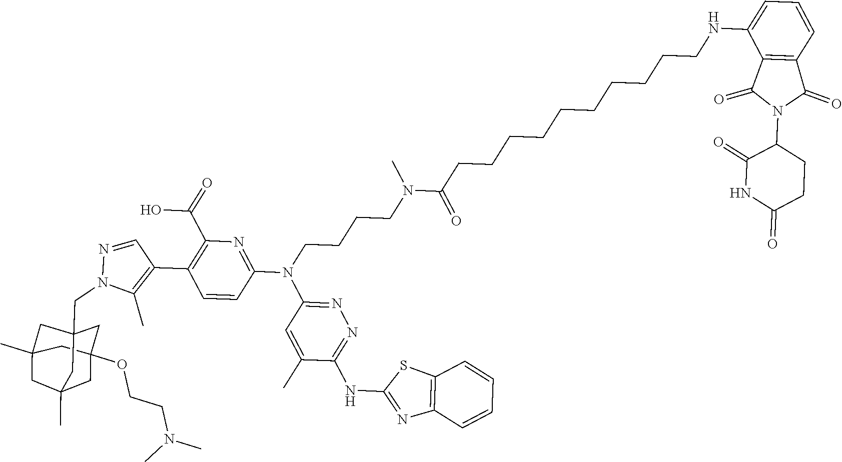 [SMILES: Cc1cc(N(CCCCN(C)C(=O)CCCCCCCCCCNc2cccc3c2C(=O)N(C2CCC(=O)NC2=O)C3=O)c2ccc(-c3cnn(CC45CC6(C)CC(C)(C4)CC(OCCN(C)C)(C6)C5)c3C)c(C(=O)O)n2)nnc1Nc1nc2ccccc2s1] |
| | 6-[[6-(1,3-benzothiazol-2-ylamino)-5-methyl-pyridazin-3-yl]-[4-[11-[[2-(2,6- |
| | dioxo-3-piperidyl)-1,3-dioxo-isoindolin-4-yl]amino]undecanoyl-methyl- |
| | amino]butyl]amino]-3-[1-[3-[2-(dimethylamino)ethoxy]-5,7-dimethyl-1- |
| | adamantyl]methyl]-5-methyl-pyrazol-4-yl]pyridine-2-carboxylic acid |
| 91 | 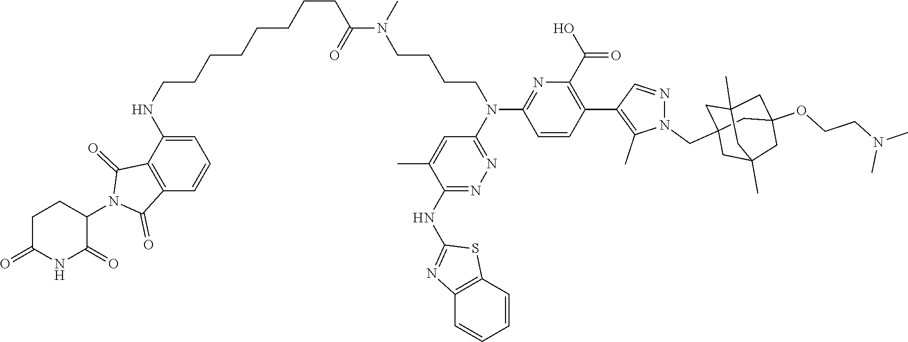 [SMILES: Cc1cc(N(CCCCN(C)C(=O)CCCCCCCCNc2cccc3c2C(=O)N(C2CCC(=O)NC2=O)C3=O)c2ccc(-c3cnn(CC45CC6(C)CC(C)(C4)CC(OCCN(C)C)(C6)C5)c3C)c(C(=O)O)n2)nnc1Nc1nc2ccccc2s1] |
| | 6-[[6-(1,3-benzothiazol-2-ylamino)-5-methyl-pyridazin-3-yl]-[4-[9-[[2-(2,6- |
| | dioxo-3-piperidyl)-1,3-dioxo-isoindolin-4-yl]amino]nonanoyl-methyl- |
| | amino]butyl]amino]-3-[1-[[3-[2-(dimethylamino)ethoxy]-5,7-dimethyl-1- |
| | adamantyl]methyl]-5-methyl-pyrazol-4-yl]pyridine-2-carboxylic acid |
| 92 | 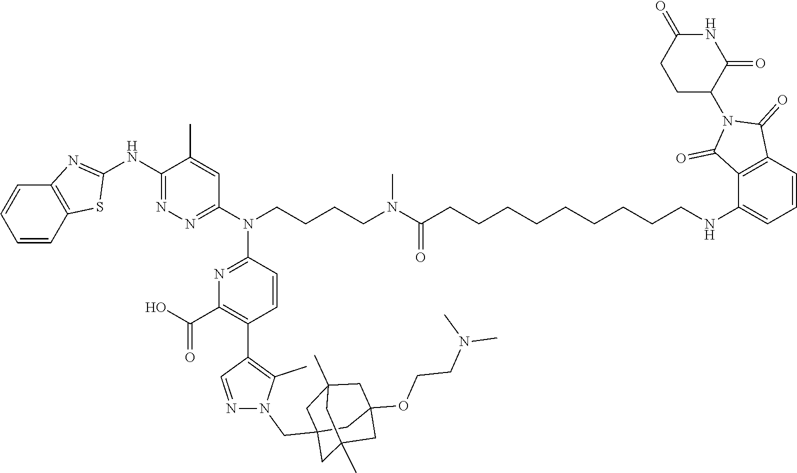 [SMILES: Cc1cc(N(CCCCN(C)C(=O)CCCCCCCCCNc2cccc3c2C(=O)N(C2CCC(=O)NC2=O)C3=O)c2ccc(-c3cnn(CC45CC6(C)CC(C)(C4)CC(OCCN(C)C)(C6)C5)c3C)c(C(=O)O)n2)nnc1Nc1nc2ccccc2s1] |
| | 6-[[6-(1,3-benzothiazol-2-ylamino)-5-methyl-pyridazin-3-yl]-[4-[10-[[2-(2,6- |
| | dioxo-3-piperidyl)-1,3-dioxo-isoindolin-4-yl]amino]decanoyl-methyl- |
| | amino]butyl]amino]-3-[1-[[3-[2-(dimethylamino)ethoxy]-5,7-dimethyl-1- |
| | adamantyl]methyl]-5-methyl-pyrazol-4-yl]pyridine-2-carboxylic acid |
| 93 | 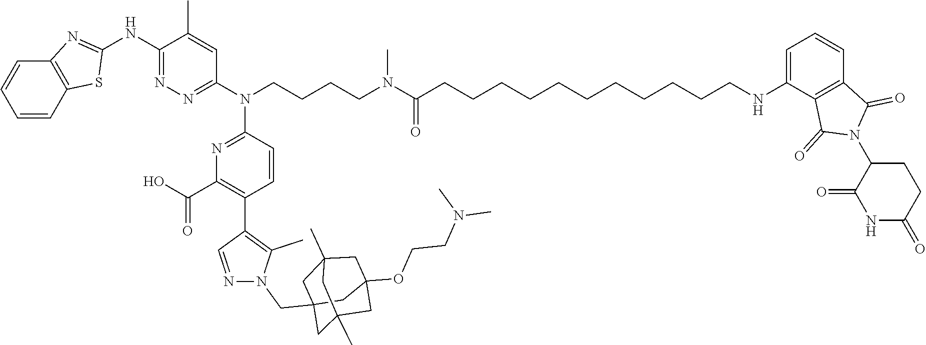 [SMILES: Cc1cc(N(CCCCN(C)C(=O)CCCCCCCCCCCNc2cccc3c2C(=O)N(C2CCC(=O)NC2=O)C3=O)c2ccc(-c3cnn(CC45CC6(C)CC(C)(C4)CC(OCCN(C)C)(C6)C5)c3C)c(C(=O)O)n2)nnc1Nc1nc2ccccc2s1] |
| | 6-[[6-(1,3-benzothiazol-2-ylamino)-5-methyl-pyridazin-3-yl]-[4-[12-[[2-(2,6- |
| | dioxo-3-piperidyl)-1,3-dioxo-isoindolin-4-yl]amino]dodecanoyl-methyl- |
| | amino]butyl]amino]-3-[1-[[3-[2-(dimethylamino)ethoxy]-5,7-dimethyl-1- |
| | adamantyl]methyl]-5-methyl-pyrazol-4-yl]pyridine-2-carboxylic acid |
| 94 | 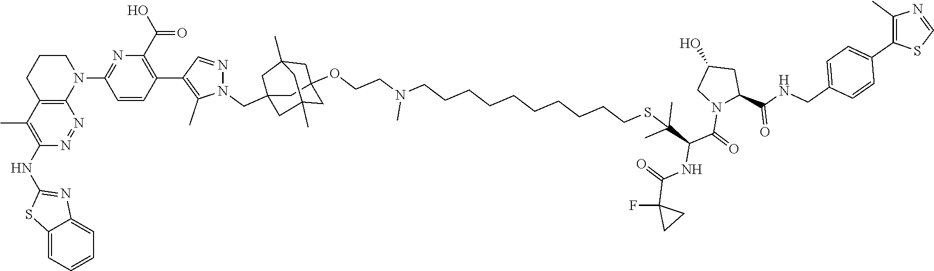 [SMILES: Cc1ncsc1-c1ccc(CNC(=O)[C@@H]2C[C@@H](O)CN2C(=O)[C@@H](NC(=O)C2(F)CC2)C(C)(C)SCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)cc1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[10-[(2R)-2-[(1- |
| | fluorocyclopropanecarbonyl)amino]-3-[(2S,4R)-4-hydroxy-2-[[4-(4- |
| | methylthiazol-5-yl)phenyl]methylcarbamoyl]pyrrolidin-1-yl]-1,1-dimethyl-3- |
| | oxo-propyl]sulfanyldecyl-methyl-amino]ethoxy]-5,7-dimethyl-1- |
| | adamantyl]methyl]-5-methyl-pyrazol-4-yl]pyridine-2-carboxylic acid |
| 95 | 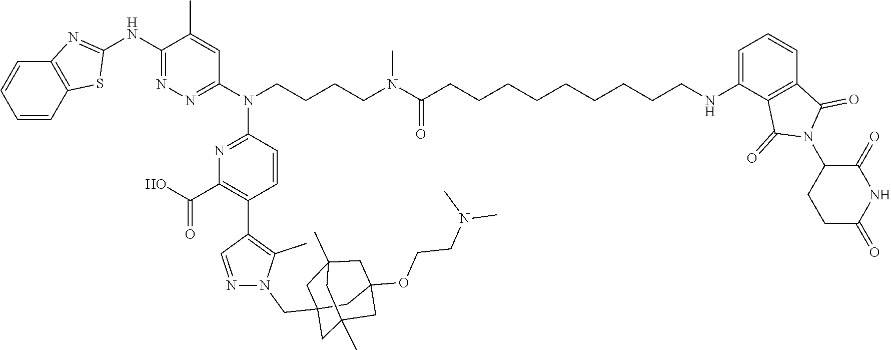 [SMILES: Cc1cc(N(CCCCN(C)C(=O)CCCCCCCCCNc2cccc3c2C(=O)N(C2CCC(=O)NC2=O)C3=O)c2ccc(-c3cnn(CC45CC6(C)CC(C)(C4)CC(OCCN(C)C)(C6)C5)c3C)c(C(=O)O)n2)nnc1Nc1nc2ccccc2s1] |
| | 6-[[6-(1,3-benzothiazol-2-ylamino)-5-methyl-pyridazin-3-yl]-[4-[10-[2-(2,6- |
| | dioxo-3-piperidyl)-1,3-dioxo-isoindolin-4-yl]oxydecanoyl-methyl- |
| | amino]butyl]amino]-3-[1-[[3-[2-(dimethylamino)ethoxy]-5,7-dimethyl-1- |
| | adamantyl]methyl]-5-methyl-pyrazol-4-yl]pyridine-2-carboxylic acid |
| 96 | 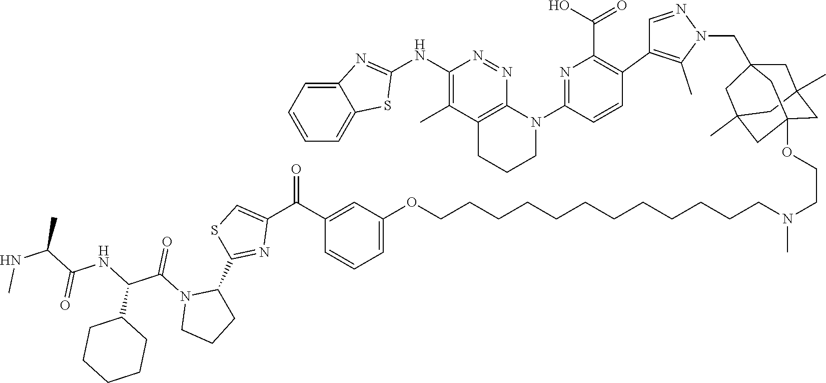 |
| | 6-[3-(1,3-benzothiazol-2-ylamino)-4-methyl-6,7-dihydro-5H-pyrido[2,3- |
| | c]pyridazin-8-yl]-3-[1-[[3-[2-[12-[3-[2-[(2S)-1-[(2S)-2-cyclohexyl-2-[[(2S)-2- |
| | (methylamino)propanoyl]amino]acetyl]pyrrolidin-2-yl]thiazole-4- |
| | carbonyl]phenoxy]dodecyl-methyl-amino]ethoxy]-5,7-dimethyl-1- |
| | adamantyl]methyl]-5-methyl-pyrazol-4-yl]pyridine-2-carboxylic acid |
| 97 | 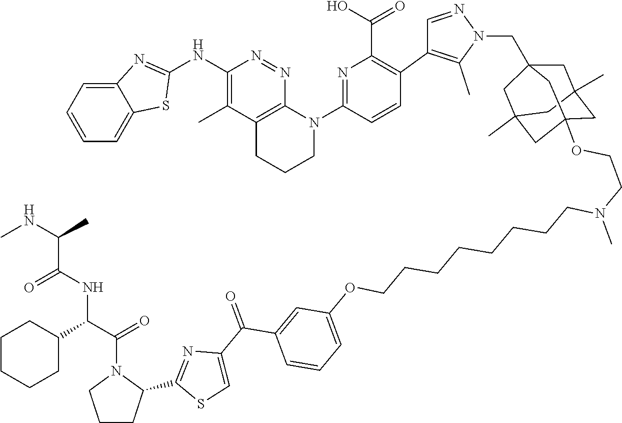 [SMILES: CN[C@@H](C)C(=O)N[C@H](C(=O)N1CCC[C@H]1c1nc(C(=O)c2cccc(OCCCCCCCCN(C)CCOC34CC5(C)CC(C)(CC(Cn6ncc(-c7ccc(N8CCCc9c8nnc(Nc8nc%10ccccc%10s8)c9C)nc7C(=O)O)c6C)(C5)C3)C4)c2)cs1)C1CCCCC1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[8-[3-[2-[(2S)-1-[(2S)-2-cyclohexyl-2-[[(2S)-2- |
| | (methylamino)propanoyl]amino]acetyl]pyrrolidin-2-yl]thiazole-4- |
| | carbonyl]phenoxy]octyl-methyl-amino]ethoxy]-5,7-dimethyl-1- |
| | adamantyl]methyl]-5-methyl-pyrazol-4-yl]pyridine-2-carboxylic acid |
| 98 | 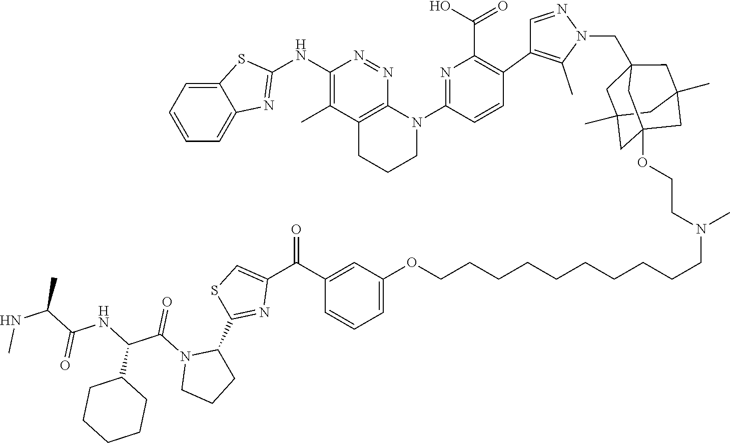 |
| | 6-[3-(1,3-benzothiazol-2-ylamino)-4-methyl-6,7-dihydro-5H-pyrido[2,3- |
| | c]pyridazin-8-yl]-3-[1-[[3-[2-[10-[3-[2-[(2S)-1-[(2S)-2-cyclohexyl-2-[[(2S)-2- |
| | (methylamino)propanoyl]amino]acetyl]pyrrolidin-2-yl]thiazole-4- |
| | carbonyl]phenoxy]decyl-methyl-amino]ethoxy]-5,7-dimethyl-1- |
| | adamantyl]methyl]-5-methyl-pyrazol-4-yl]pyridine-2-carboxylic acid |
| 99 | 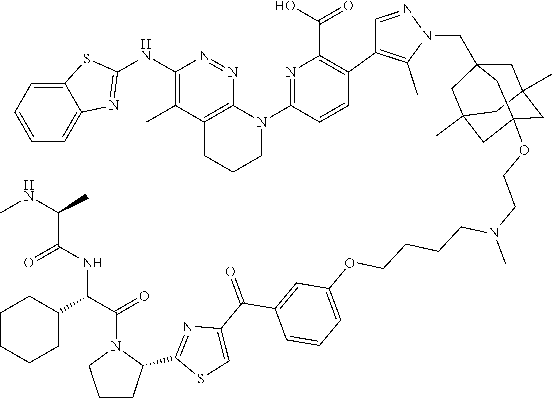 |
| | 6-[3-(1,3-benzothiazol-2-ylamino)-4-methyl-6,7-dihydro-5H-pyrido[2,3- |
| | c]pyridazin-8-yl]-3-[1-[[3-[2-[4-[3-[2-[(2S)-1-[(2S)-2-cyclohexyl-2-[[(2S)-2- |
| | (methylamino)propanoyl]amino]acetyl]pyrrolidin-2-yl]thiazole-4- |
| | carbonyl]phenoxy]butyl-methyl-amino]ethoxy]-5,7-dimethyl-1- |
| | adamantyl]methyl]-5-methyl-pyrazol-4-yl]pyridine-2-carboxylic acid |
| 100 | 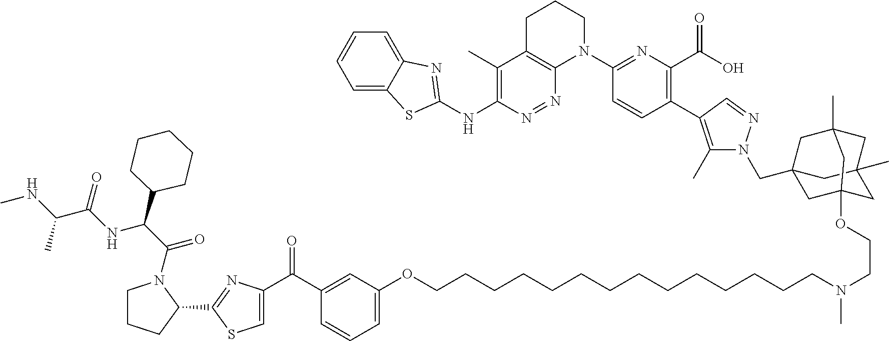 |
| | 6-[3-(1,3-benzothiazol-2-ylamino)-4-methyl-6,7-dihydro-5H-pyrido[2,3- |
| | c]pyridazin-8-yl]-3-[1-[[3-[2-[14-[3-[2-[(2S)-1-[(2S)-2-cyclohexyl-2-[[(2S)-2- |
| | (methylamino)propanoyl]amino]acetyl]pyrrolidin-2-yl]thiazole-4- |
| | carbonyl]phenoxy]tetradecyl-methyl-amino]ethoxy]-5,7-dimethyl-1- |
| | adamantyl]methyl]-5-methyl-pyrazol-4-yl]pyridine-2-carboxylic acid |
| 101 | 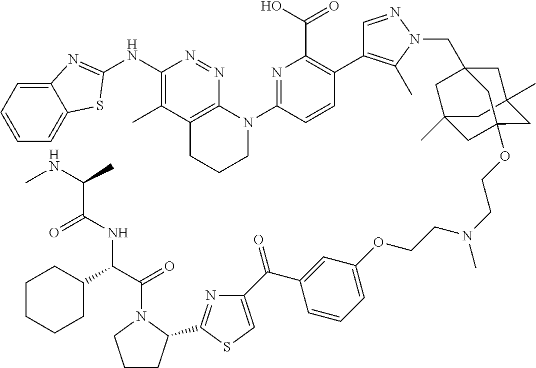 |
| | 6-[3-(1,3-benzothiazol-2-ylamino)-4-methyl-6,7-dihydro-5H-pyrido[2,3- |
| | c]pyridazin-8-yl]-3-[1-[[3-[2-[2-[3-[2-[(2S)-1-[(2S)-2-cyclohexyl-2-[[(2S)-2- |
| | (methylamino)propanoyl]amino]acetyl]pyrrolidin-2-yl]thiazole-4- |
| | carbonyl]phenoxy]ethyl-methyl-amino]ethoxy]-5,7-dimethyl-1- |
| | adamantyl]methyl]-5-methyl-pyrazol-4-yl]pyridine-2-carboxylic acid |
| 102 | 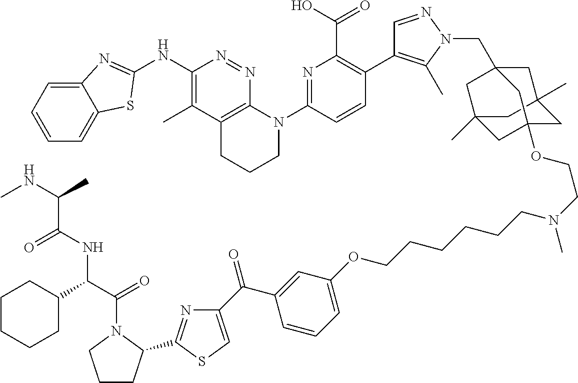 |
| | 6-[3-(1,3-benzothiazol-2-ylamino)-4-methyl-6,7-dihydro-5H-pyrido[2,3- |
| | c]pyridazin-8-yl]-3-[1-[[3-[2-[6-[3-[2-[(2S)-1-[(2S)-2-cyclohexyl-2-[[(2S)-2- |
| | (methylamino)propanoyl]amino]acetyl]pyrrolidin-2-yl]thiazole-4- |
| | carbonyl]phenoxy]hexyl-methyl-amino]ethoxy]-5,7-dimethyl-1- |
| | adamantyl]methyl]-5-methyl-pyrazol-4-yl]pyridine-2-carboxylic acid |
| 103 | 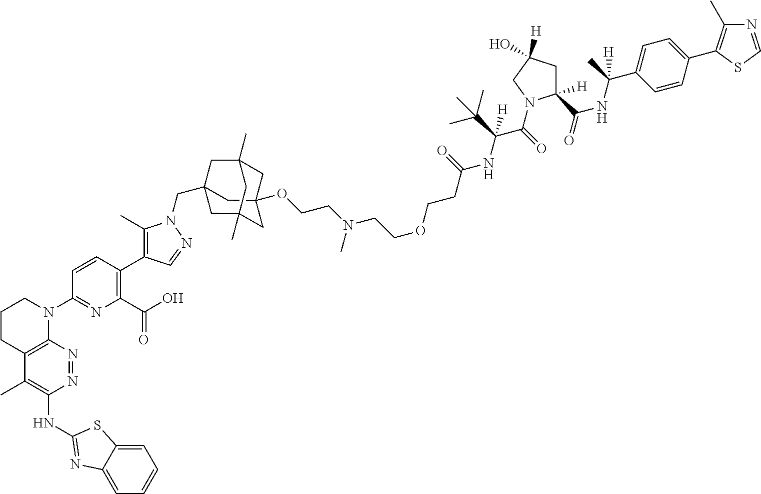 [SMILES: Cc1ncsc1-c1ccc([C@H](C)NC(=O)[C@@H]2C[C@@H](O)CN2C(=O)[C@@H](NC(=O)CCOCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[2-[3-[[(1S)-1-[(2S,4R)-4-hydroxy-2-[[(1S)-1-[4- |
| | (4-methylthiazol-5-yl)phenyl]ethyl]carbamoyl]pyrrolidine-1-carbonyl]-2,2- |
| | dimethyl-propyl]amino]-3-oxo-propoxy]ethyl-methyl-amino]ethoxy]-5,7- |
| | dimethyl-1-adamantyl]methyl]-5-methyl-pyrazol-4-yl]pyridine-2-carboxylic |
| | acid |
| 104 | 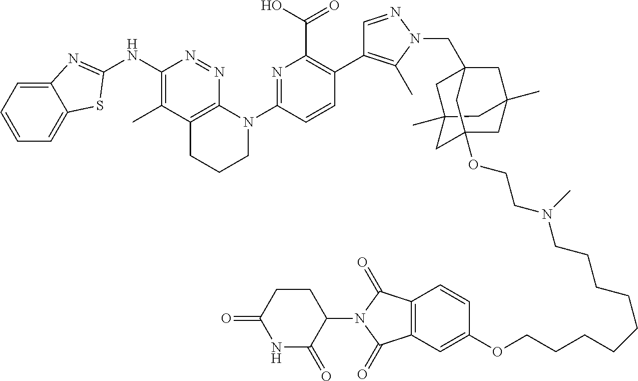 |
| | 6-[3-(1,3-benzothiazol-2-ylamino)-4-methyl-6,7-dihydro-5H-pyrido[2,3- |
| | c]pyridazin-8-yl]-3-[1-[[3-[2-[9-[2-(2,6-dioxo-3-piperidyl)-1,3-dioxo-isoindolin- |
| | 5-yl]oxynonyl-methyl-amino]ethoxy]-5,7-dimethyl-1-adamantyl]methyl]-5- |
| | methyl-pyrazol-4-yl]pyridine-2-carboxylic acid |
| 105 | 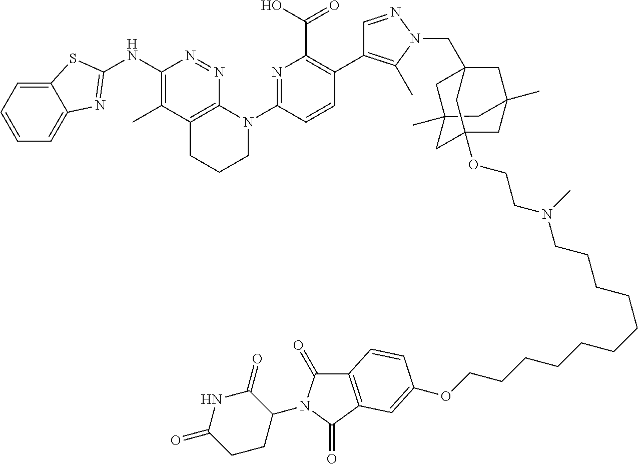 |
| | 6-[3-(1,3-benzothiazol-2-ylamino)-4-methyl-6,7-dihydro-5H-pyrido[2,3- |
| | c]pyridazin-8-yl]-3-[1-[[3-[2-[11-[2-(2,6-dioxo-3-piperidyl)-1,3-dioxo- |
| | isoindolin-5-yl]oxyundecyl-methyl-amino]ethoxy]-5,7-dimethyl-1- |
| | adamantyl]methyl]-5-methyl-pyrazol-4-yl]pyridine-2-carboxylic acid |
| 106 | 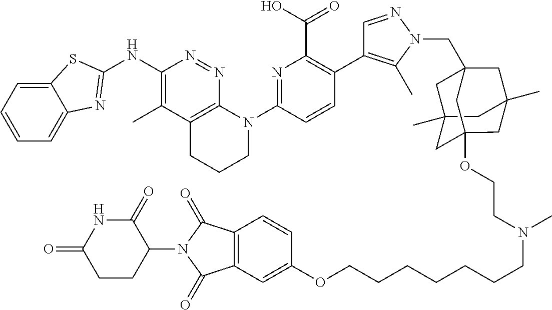 |
| | 6-[3-(1,3-benzothiazol-2-ylamino)-4-methyl-6,7-dihydro-5H-pyrido[2,3- |
| | c]pyridazin-8-yl]-3-[1-[[3-[2-[7-[2-(2,6-dioxo-3-piperidyl)-1,3-dioxo-isoindolin- |
| | 5-yl]oxyheptyl-methyl-amino]ethoxy]-5,7-dimethyl-1-adamantyl]methyl]-5- |
| | methyl-pyrazol-4-yl]pyridine-2-carboxylic acid |
| 107 | 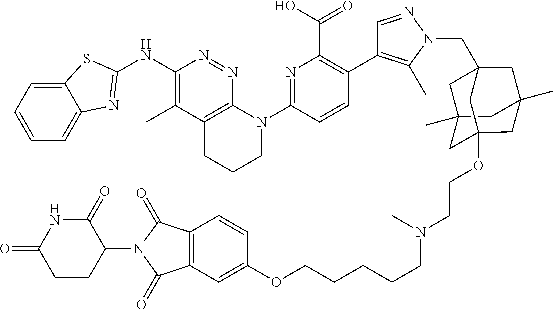 |
| | 6-[3-(1,3-benzothiazol-2-ylamino)-4-methyl-6,7-dihydro-5H-pyrido[2,3- |
| | c]pyridazin-8-yl]-3-[1-[[3-[2-[5-[2-(2,6-dioxo-3-piperidyl)-1,3-dioxo-isoindolin- |
| | 5-yl]oxypentyl-methyl-amino]ethoxy]-5,7-dimethyl-1-adamantyl]methyl]-5- |
| | methyl-pyrazol-4-yl]pyridine-2-carboxylic acid |
| 108 | 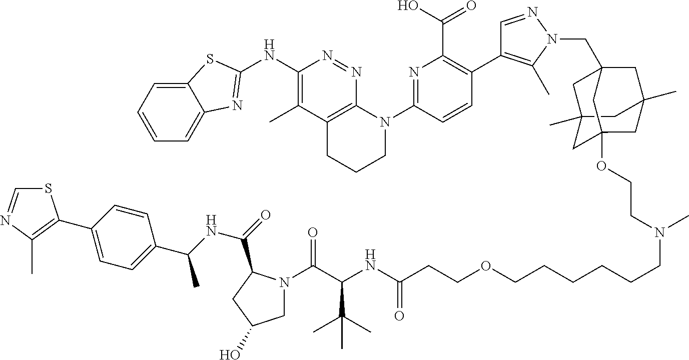 |
| | 6-[3-(1,3-benzothiazol-2-ylamino)-4-methyl-6,7-dihydro-5H-pyrido[2,3- |
| | c]pyridazin-8-yl]-3-[1-[[3-[2-[6-[3-[[(1S)-1-[(2S,4R)-4-hydroxy-2-[[(1S)-1-[4- |
| | (4-methylthiazol-5-yl)phenyl]ethyl]carbamoyl]pyrrolidine-1-carbonyl]-2,2- |
| | dimethyl-propyl]amino]-3-oxo-propoxy]hexyl-methyl-amino]ethoxy]-5,7- |
| | dimethyl-1-adamantyl]methyl]-5-methyl-pyrazol-4-yl]pyridine-2-carboxylic |
| | acid |
| 109 | 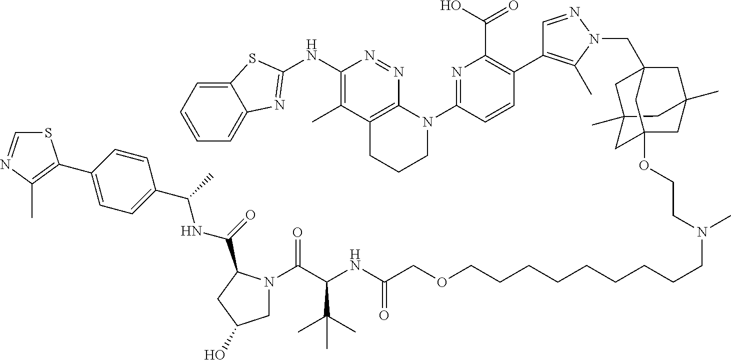 |
| | 6-[3-(1,3-benzothiazol-2-ylamino)-4-methyl-6,7-dihydro-5H-pyrido[2,3- |
| | c]pyridazin-8-yl]-3-[1-[[3-[2-[9-[2-[[(1S)-1-[(2S,4R)-4-hydroxy-2-[[(1S)-1-[4- |
| | (4-methylthiazol-5-yl)phenyl]ethyl]carbamoyl]pyrrolidine-1-carbonyl]-2,2- |
| | dimethyl-propyl]amino]-2-oxo-ethoxy]nonyl-methyl-amino]ethoxy]-5,7- |
| | dimethyl-1-adamantyl]methyl]-5-methyl-pyrazol-4-yl]pyridine-2-carboxylic |
| | acid |
| 110 | 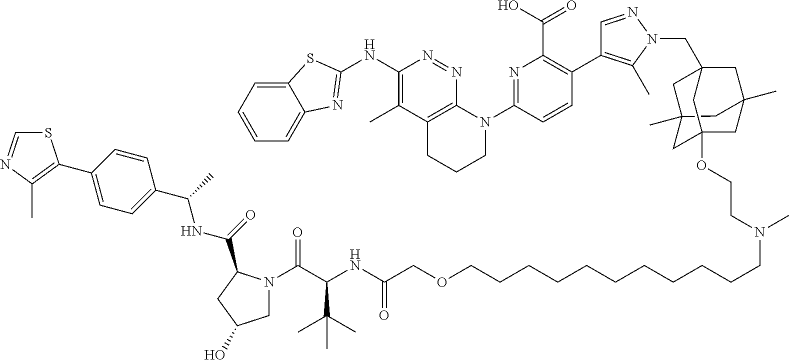 |
| | 6-[3-(1,3-benzothiazol-2-ylamino)-4-methyl-6,7-dihydro-5H-pyrido[2,3- |
| | c]pyridazin-8-yl]-3-[1-[[3-[2-[11-[2-[[(1S)-1-[(2S,4R)-4-hydroxy-2-[[(1S)-1-[4- |
| | (4-methylthiazol-5-yl)phenyl]ethyl]carbamoyl]pyrrolidine-1-carbonyl]-2,2- |
| | dimethyl-propyl]amino]-2-oxo-ethoxy]undecyl-methyl-amino]ethoxy]-5,7- |
| | dimethyl-1-adamantyl]methyl]-5-methyl-pyrazol-4-yl]pyridine-2-carboxylic |
| | acid |
| 111 | 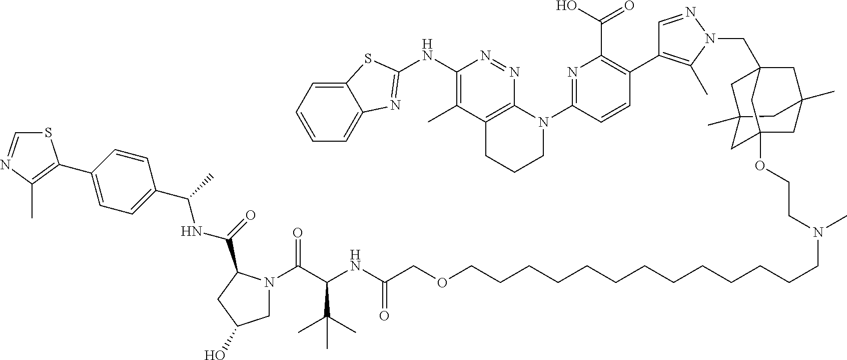 |
| | 6-[3-(1,3-benzothiazol-2-ylamino)-4-methyl-6,7-dihydro-5H-pyrido[2,3- |
| | c]pyridazin-8-yl]-3-[1-[[3-[2-[13-[2-[[(1S)-1-[(2S,4R)-4-hydroxy-2-[[(1S)-1-[4- |
| | (4-methylthiazol-5-yl)phenyl]ethyl]carbamoyl]pyrrolidine-1-carbonyl]-2,2- |
| | dimethyl-propyl]amino]-2-oxo-ethoxy]tridecyl-methyl-amino]ethoxy]-5,7- |
| | dimethyl-1-adamantyl]methyl]-5-methyl-pyrazol-4-yl]pyridine-2-carboxylic |
| | acid |
| 112 | 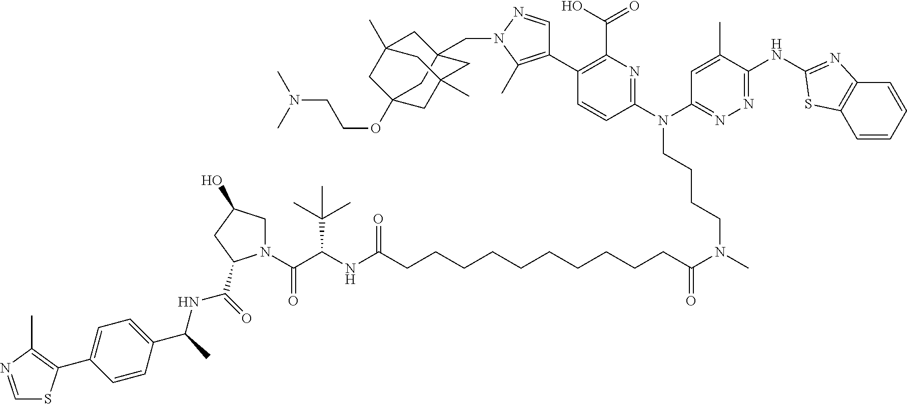 [SMILES: Cc1cc(N(CCCCN(C)C(=O)CCCCCCCCCCC(=O)N[C@H](C(=O)N2C[C@H](O)C[C@H]2C(=O)N[C@@H](C)c2ccc(-c3scnc3C)cc2)C(C)(C)C)c2ccc(-c3cnn(CC45CC6(C)CC(C)(C4)CC(OCCN(C)C)(C6)C5)c3C)c(C(=O)O)n2)nnc1Nc1nc2ccccc2s1] |
| | 6-[[6-(1,3-benzothiazol-2-ylamino)-5-methyl-pyridazin-3-yl]-[4-[[12-[[(1S)-1- |
| | [(2S,4R)-4-hydroxy-2-[[(1S)-1-[4-(4-methylthiazol-5- |
| | yl)phenyl]ethyl]carbamoyl]pyrrolidine-1-carbonyl]-2,2-dimethyl- |
| | propyl]amino]-12-oxo-dodecanoyl]-methyl-amino]butyl]amino]-3-[1-[[3-[2- |
| | (dimethylamino)ethoxy]-5,7-dimethyl-1-adamantyl]methyl]-5-methyl- |
| | pyrazol-4-yl]pyridine-2-carboxylic acid |
| 113 | 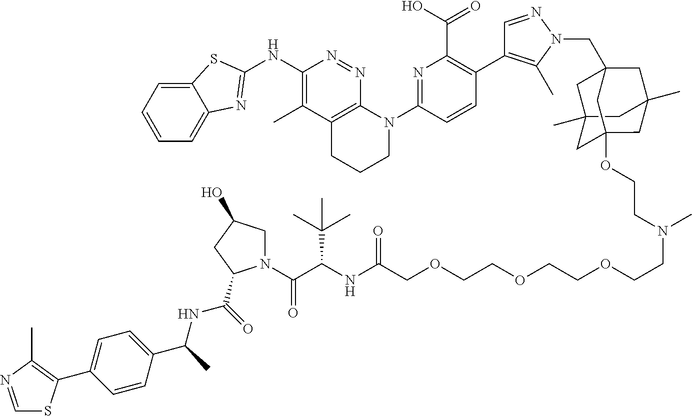 |
| | 6-[3-(1,3-benzothiazol-2-ylamino)-4-methyl-6,7-dihydro-5H-pyrido[2,3- |
| | c]pyridazin-8-yl]-3-[1-[[3-[2-[2-[2-[2-[2-[[(1S)-1-[(2S,4R)-4-hydroxy-2-[[(1S)- |
| | 1-[4-(4-methylthiazol-5-yl)phenyl]ethyl]carbamoyl]pyrrolidine-1-carbonyl]- |
| | 2,2-dimethyl-propyl]amino]-2-oxo-ethoxy]ethoxy]ethoxy]ethyl-methyl- |
| | amino]ethoxy]-5,7-dimethyl-1-adamantyl]methyl]-5-methyl-pyrazol-4- |
| | yl]pyridine-2-carboxylic acid |
| 114 | 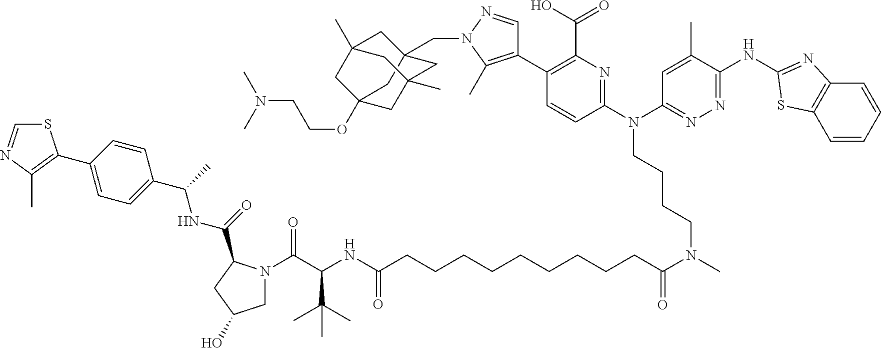 |
| | 6-[[6-(1,3-benzothiazol-2-ylamino)-5-methyl-pyridazin-3-yl]-[4-[[11-[[(1S)-1- |
| | [(2S,4R)-4-hydroxy-2-[[(1S)-1-[4-(4-methylthiazol-5- |
| | yl)phenyl]ethyl]carbamoyl]pyrrolidine-1-carbonyl]-2,2-dimethyl- |
| | propyl]amino]-11-oxo-undecanoyl]-methyl-amino]butyl]amino]-3-[1-[[3-[2- |
| | (dimethylamino)ethoxy]-5,7-dimethyl-1-adamantyl]methyl]-5-methyl- |
| | pyrazol-4-yl]pyridine-2-carboxylic acid |
| 115 | 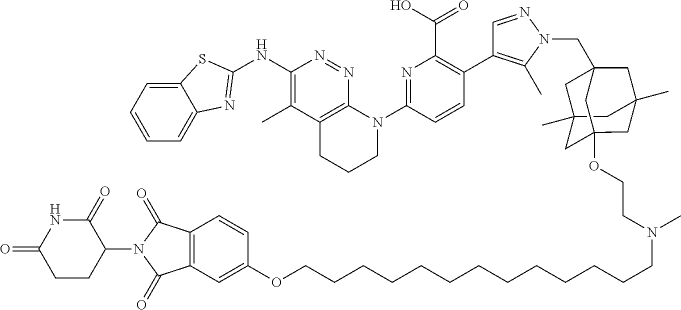 |
| | 6-[3-(1,3-benzothiazol-2-ylamino)-4-methyl-6,7-dihydro-5H-pyrido[2,3- |
| | c]pyridazin-8-yl]-3-[1-[[3-[2-[13-[2-(2,6-dioxo-3-piperidyl)-1,3-dioxo- |
| | isoindolin-5-yl]oxytridecyl-methyl-amino]ethoxy]-5,7-dimethyl-1- |
| | adamantyl]methyl]-5-methyl-pyrazol-4-yl]pyridine-2-carboxylic acid |
| 116 | 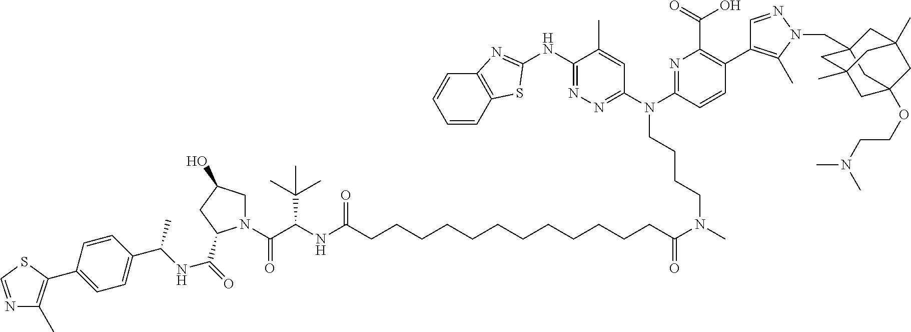 |
| | 6-[[6-(1,3-benzothiazol-2-ylamino)-5-methyl-pyridazin-3-yl]-[4-[[14-[[(1S)-1- |
| | [(2S,4R)-4-hydroxy-2-[[(1S)-1-[4-(4-methylthiazol-5- |
| | yl)phenyl]ethyl]carbamoyl]pyrrolidine-1-carbonyl]-2,2-dimethyl- |
| | propyl]amino]-14-oxo-tetradecanoyl]-methyl-amino]butyl]amino]-3-[1-[3-[2- |
| | (dimethylamino)ethoxy]-5,7-dimethyl-1-adamantyl]methyl]-5-methyl- |
| | pyrazol-4-yl]pyridine-2-carboxylic acid |
| 117 | 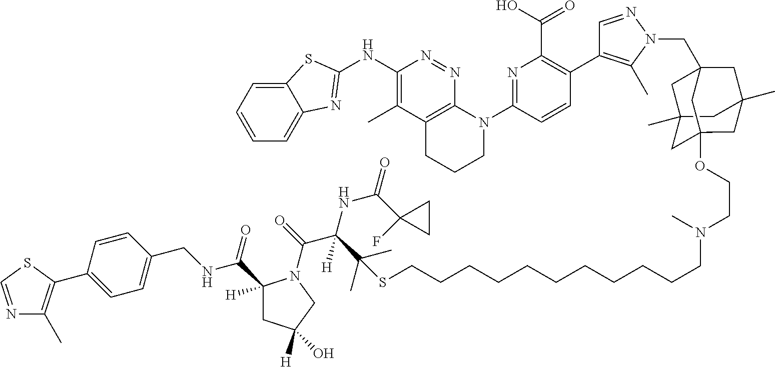 |
| | 6-[3-(1,3-benzothiazol-2-ylamino)-4-methyl-6,7-dihydro-5H-pyrido[2,3- |
| | c]pyridazin-8-yl]-3-[1-[[3-[2-[11-[(2R)-2-[(1- |
| | fluorocyclopropanecarbonyl)amino]-3-[(2S,4R)-4-hydroxy-2-[[4-(4- |
| | methylthiazol-5-yl)phenyl]methylcarbamoyl]pyrrolidin-1-yl]-1,1-dimethyl-3- |
| | oxo-propyl]sulfanylundecyl-methyl-amino]ethoxy]-5,7-dimethyl-1- |
| | adamantyl]methyl]-5-methyl-pyrazol-4-yl]pyridine-2-carboxylic acid |
| 118 | 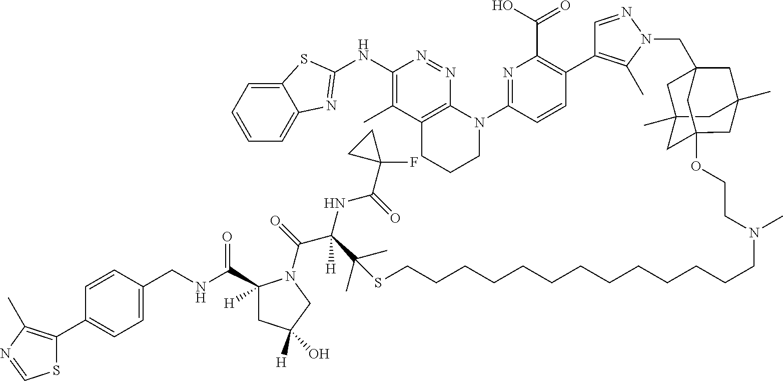 |
| | 6-[3-(1,3-benzothiazol-2-ylamino)-4-methyl-6,7-dihydro-5H-pyrido[2,3- |
| | c]pyridazin-8-yl]-3-[1-[[3-[2-[13-[(2R)-2-[(1- |
| | fluorocyclopropanecarbonyl)amino]-3-[(2S,4R)-4-hydroxy-2-[[4-(4- |
| | methylthiazol-5-yl)phenyl]methylcarbamoyl]pyrrolidin-1-yl]-1,1-dimethyl-3- |
| | oxo-propyl]sulfanyltridecyl-methyl-amino]ethoxy]-5,7-dimethyl-1- |
| | adamantyl]methyl]-5-methyl-pyrazol-4-yl]pyridine-2-carboxylic acid |
| 119 | 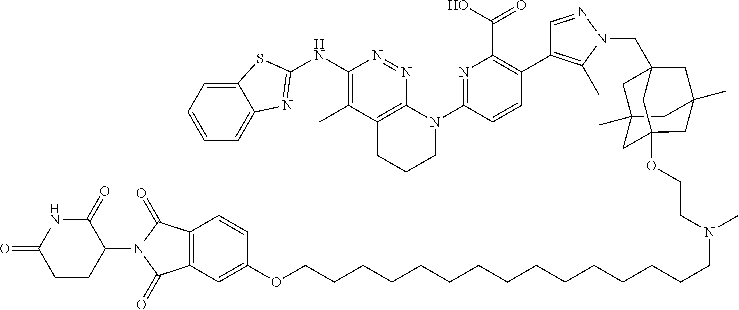 |
| | 6-[3-(1,3-benzothiazol-2-ylamino)-4-methyl-6,7-dihydro-5H-pyrido[2,3- |
| | c]pyridazin-8-yl]-3-[1-[[3-[2-[15-[2-(2,6-dioxo-3-piperidyl)-1,3-dioxo- |
| | isoindolin-5-yl]oxypentadecyl-methyl-amino]ethoxy]-5,7-dimethyl-1- |
| | adamantyl]methyl]-5-methyl-pyrazol-4-yl]pyridine-2-carboxylic acid |
| 120 | 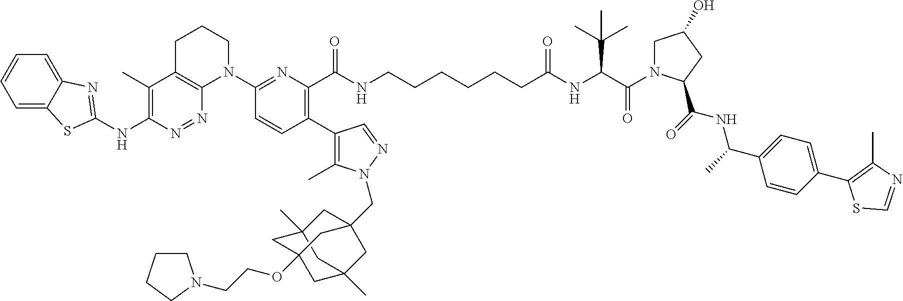 [SMILES: Cc1ncsc1-c1ccc([C@H](C)NC(=O)[C@@H]2C[C@@H](O)CN2C(=O)[C@@H](NC(=O)CCCCCCNC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C(C)(C)C)cc1] |
| | 6-[3-(1,3-benzothiazol-2-ylamino)-4-methyl-6,7-dihydro-5H-pyrido[2,3- |
| | c]pyridazin-8-yl]-3-[1-[[3,5-dimethyl-7-(2-pyrrolidin-1-ylethoxy)-1- |
| | adamantyl]methyl]-5-methyl-pyrazol-4-yl]-N-[7-[[(1S)-1-[(2S,4R)-4-hydroxy- |
| | 2-[(1S)-1-[4-(4-methylthiazol-5-yl)phenyl]ethyl]carbamoyl]pyrrolidine-1- |
| | carbonyl]-2,2-dimethyl-propyl]amino]-7-oxo-heptyl]pyridine-2-carboxamide |
| 121 | 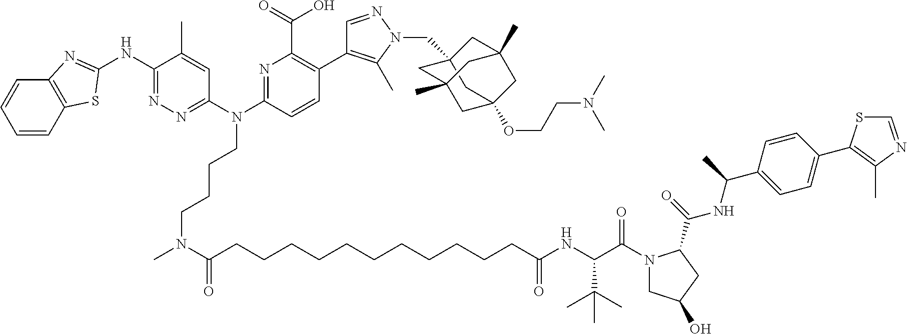 [SMILES: Cc1cc(N(CCCCN(C)C(=O)CCCCCCCCCCCC(=O)N[C@H](C(=O)N2C[C@H](O)C[C@H]2C(=O)N[C@@H](C)c2ccc(-c3scnc3C)cc2)C(C)(C)C)c2ccc(-c3cnn(C[C@]45C[C@]6(C)C[C@](C)(C4)C[C@@](OCCN(C)C)(C6)C5)c3C)c(C(=O)O)n2)nnc1Nc1nc2ccccc2s1] |
| | 6-[[6-(1,3-benzothiazol-2-ylamino)-5-methyl-pyridazin-3-yl]-[4-[[13-[[(1S)-1- |
| | [(2S,4R)-4-hydroxy-2-[[(1S)-1-[4-(4-methylthiazol-5- |
| | yl)phenyl]ethyl]carbamoyl]pyrrolidine-1-carbonyl]-2,2-dimethyl- |
| | propyl]amino]-13-oxo-tridecanoyl]-methyl-amino]butyl]amino]-3-[1-[[3-[2- |
| | (dimethylamino)ethoxy]-5,7-dimethyl-1-adamantyl]methyl]-5-methyl- |
| | pyrazol-4-yl]pyridine-2-carboxylic acid |
| 122 | 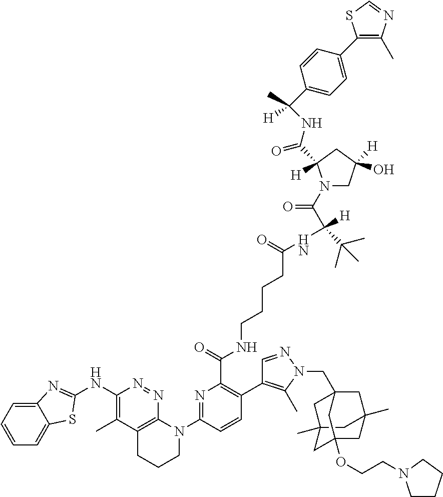 [SMILES: Cc1ncsc1-c1ccc([C@H](C)NC(=O)[C@@H]2C[C@@H](O)CN2C(=O)[C@@H](NC(=O)CCCCNC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C(C)(C)C)cc1] |
| | 6-[3-(1,3-benzothiazol-2-ylamino)-4-methyl-6,7-dihydro-5H-pyrido[2,3- |
| | c]pyridazin-8-yl]-3-[1-[[3,5-dimethyl-7-(2-pyrrolidin-1-ylethoxy)-1- |
| | adamantyl]methyl]-5-methyl-pyrazol-4-yl]-N-[5-[[(1S)-1-[(2S,4R)-4-hydroxy- |
| | 2-[[(1S)-1-[4-(4-methylthiazol-5-yl)phenyl]ethyl]carbamoyl]pyrrolidine-1- |
| | carbonyl]-2,2-dimethyl-propyl]amino]-5-oxo-pentyl]pyridine-2-carboxamide |
| 123 | 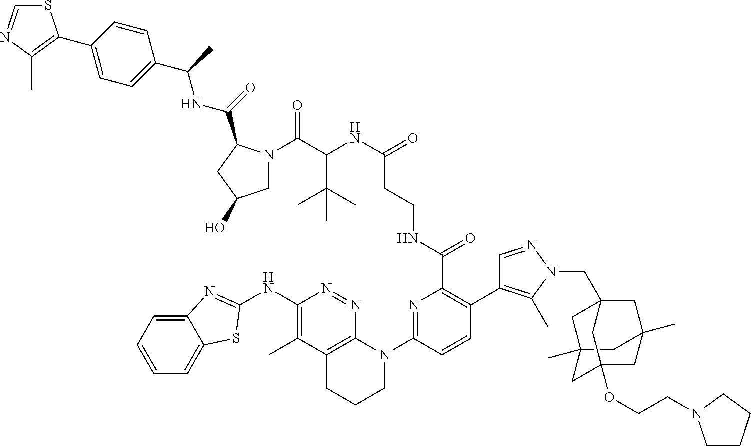 [SMILES: Cc1ncsc1-c1ccc([C@@H](C)NC(=O)[C@@H]2C[C@H](O)CN2C(=O)C(NC(=O)CCNC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C(C)(C)C)cc1] |
| | 6-[3-(1,3-benzothiazol-2-ylamino)-4-methyl-6,7-dihydro-5H-pyrido[2,3- |
| | c]pyridazin-8-yl]-3-[1-[[3,5-dimethyl-7-(2-pyrrolidin-1-ylethoxy)-1- |
| | adamantyl]methyl]-5-methyl-pyrazol-4-yl]-N-[3-[[(1S)-1-[(2S,4R)-4-hydroxy- |
| | 2-[[(1S)-1-[4-(4-methylthiazol-5-yl)phenyl]ethyl]carbamoyl]pyrrolidine-1- |
| | carbonyl]-2,2-dimethyl-propyl]amino]-3-oxo-propyl]pyridine-2-carboxamide |
| 124 | 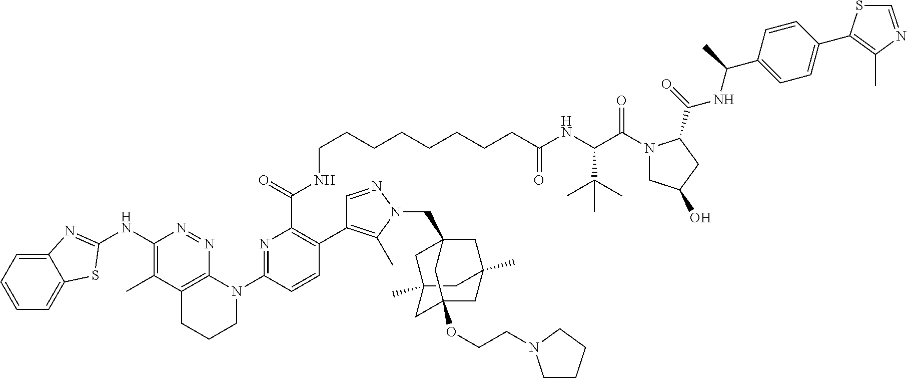 [SMILES: Cc1ncsc1-c1ccc([C@H](C)NC(=O)[C@@H]2C[C@@H](O)CN2C(=O)[C@@H](NC(=O)CCCCCCCCNC(=O)c2nc(N3CCCc4c3nnc(Nc3nc5ccccc5s3)c4C)ccc2-c2cnn(C[C@]34C[C@]5(C)C[C@](C)(C3)C[C@@](OCCN3CCCC3)(C5)C4)c2C)C(C)(C)C)cc1] |
| | 6-[3-(1,3-benzothiazol-2-ylamino)-4-methyl-6,7-dihydro-5H-pyrido[2,3- |
| | c]pyridazin-8-yl]-3-[1-[[3,5-dimethyl-7-(2-pyrrolidin-1-ylethoxy)-1- |
| | adamantyl]methyl]-5-methyl-pyrazol-4-yl]-N-[9-[[(1S)-1-[(2S,4R)-4-hydroxy- |
| | 2-[[(1S)-1-[4-(4-methylthiazol-5-yl)phenyl]ethyl]carbamoyl]pyrrolidine-1- |
| | carbonyl]-2,2-dimethyl-propyl]amino]-9-oxo-nonyl]pyridine-2-carboxamide |
| 125 | 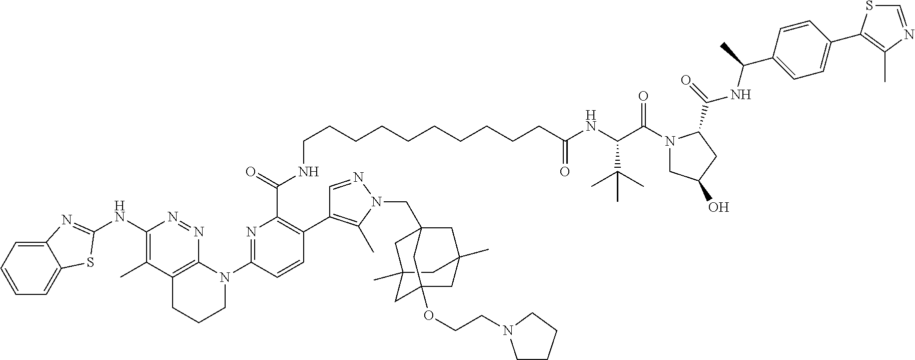 [SMILES: Cc1ncsc1-c1ccc([C@H](C)NC(=O)[C@@H]2C[C@@H](O)CN2C(=O)[C@@H](NC(=O)CCCCCCCCCCNC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C(C)(C)C)cc1] |
| | 6-[3-(1,3-benzothiazol-2-ylamino)-4-methyl-6,7-dihydro-5H-pyrido[2,3- |
| | c]pyridazin-8-yl]-3-[1-[[3,5-dimethyl-7-(2-pyrrolidin-1-ylethoxy)-1- |
| | adamantyl]methyl]-5-methyl-pyrazol-4-yl]-N-[11-[[(1S)-1-[(2S,4R)-4- |
| | hydroxy-2-[[(1S)-1-[4-(4-methylthiazol-5- |
| | yl)phenyl]ethyl]carbamoyl]pyrrolidine-1-carbonyl]-2,2-dimethyl- |
| | propyl]amino]-11-oxo-undecyl]pyridine-2-carboxamide |
| 126 | 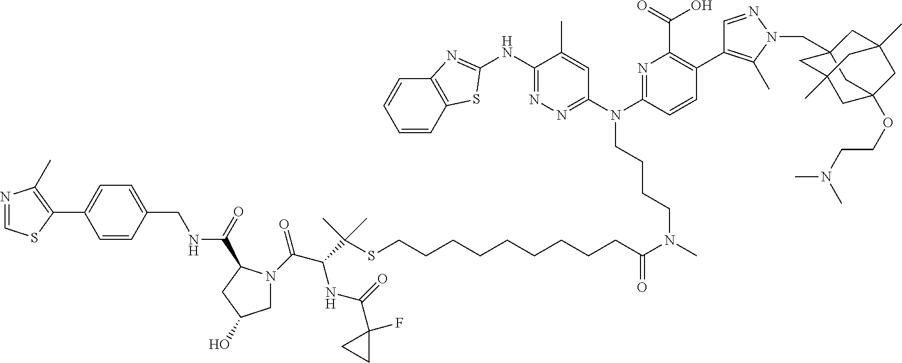 [SMILES: Cc1cc(N(CCCCN(C)C(=O)CCCCCCCCCSC(C)(C)[C@H](NC(=O)C2(F)CC2)C(=O)N2C[C@H](O)C[C@H]2C(=O)NCc2ccc(-c3scnc3C)cc2)c2ccc(-c3cnn(CC45CC6(C)CC(C)(C4)CC(OCCN(C)C)(C6)C5)c3C)c(C(=O)O)n2)nnc1Nc1nc2ccccc2s1] |
| | 6-[[6-(1,3-benzothiazol-2-ylamino)-5-methyl-pyridazin-3-yl]-[4-[10-[(2R)-2- |
| | [(1-fluorocyclopropanecarbonyl)amino]-3-[(2S,4R)-4-hydroxy-2-[[4-(4- |
| | methylthiazol-5-yl)phenyl]methylcarbamoyl]pyrrolidin-1-yl]-1,1-dimethyl-3- |
| | oxo-propyl]sulfanyldecanoyl-methyl-amino]butyl]amino]-3-[1-[[3-[2- |
| | (dimethylamino)ethoxy]-5,7-dimethyl-1-adamantyl]methyl]-5-methyl- |
| | pyrazol-4-yl]pyridine-2-carboxylic acid |
| 127 | 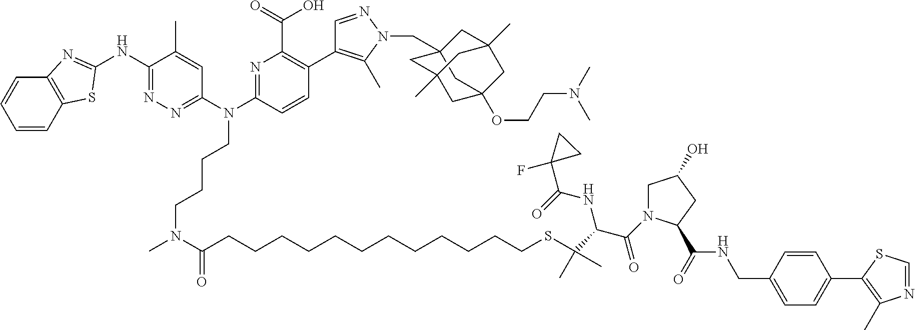 [SMILES: Cc1cc(N(CCCCN(C)C(=O)CCCCCCCCCCCCSC(C)(C)[C@H](NC(=O)C2(F)CC2)C(=O)N2C[C@H](O)C[C@H]2C(=O)NCc2ccc(-c3scnc3C)cc2)c2ccc(-c3cnn(CC45CC6(C)CC(C)(C4)CC(OCCN(C)C)(C6)C5)c3C)c(C(=O)O)n2)nnc1Nc1nc2ccccc2s1] |
| | 6-[[6-(1,3-benzothiazol-2-ylamino)-5-methyl-pyridazin-3-yl]-[4-[13-[(2R)-2- |
| | [(1-fluorocyclopropanecarbonyl)amino]-3-[(2S,4R)-4-hydroxy-2-[[4-(4- |
| | methylthiazol-5-yl)phenyl]methylcarbamoyl]pyrrolidin-1-yl]-1,1-dimethyl-3- |
| | oxo-propyl]sulfanyltridecanoyl-methyl-amino]butyl]amino]-3-[1-[[3-[2- |
| | (dimethylamino)ethoxy]-5,7-dimethyl-1-adamantyl]methyl]-5-methyl- |
| | pyrazol-4-yl]pyridine-2-carboxylic acid |
| 128 | 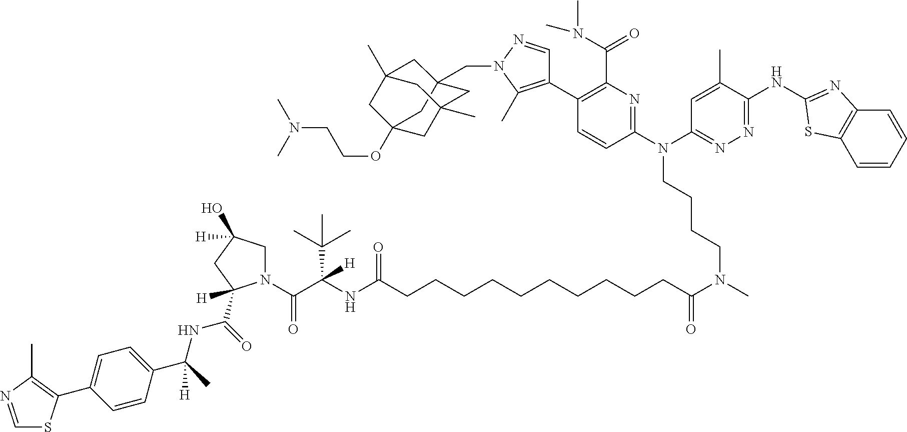 [SMILES: Cc1cc(N(CCCCN(C)C(=O)CCCCCCCCCCC(=O)N[C@H](C(=O)N2C[C@H](O)C[C@H]2C(=O)N[C@@H](C)c2ccc(-c3scnc3C)cc2)C(C)(C)C)c2ccc(-c3cnn(CC45CC6(C)CC(C)(C4)CC(OCCN(C)C)(C6)C5)c3C)c(C(=O)N(C)C)n2)nnc1Nc1nc2ccccc2s1] |
| | N′-[4-[[6-(1,3-benzothiazol-2-ylamino)-5-methyl-pyridazin-3-yl]-[5-[1-[[3-[2- |
| | (dimethylamino)ethoxy]-5,7-dimethyl-1-adamantyl]methyl]-5-methyl- |
| | pyrazol-4-yl]-6-(dimethylcarbamoyl)-2-pyridyl]amino]butyl]-N-[(1S)-1- |
| | [(2S,4R)-4-hydroxy-2-[[(1S)-1-[4-(4-methylthiazol-5- |
| | yl)phenyl]ethyl]carbamoyl]pyrrolidine-1-carbonyl]-2,2-dimethyl-propyl]-N′- |
| | methyl-dodecanediamide |
| 129 | 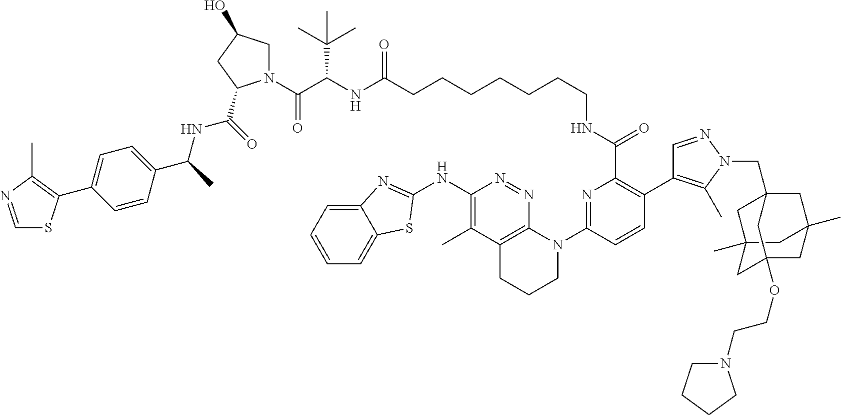 |
| | 6-[3-(1,3-benzothiazol-2-ylamino)-4-methyl-6,7-dihydro-5H-pyrido[2,3- |
| | c]pyridazin-8-yl]-3-[1-[[3,5-dimethyl-7-(2-pyrrolidin-1-ylethoxy)-1- |
| | adamantyl]methyl]-5-methyl-pyrazol-4-yl]-N-[8-[[(1S)-1-[(2S,4R)-4-hydroxy- |
| | 2-[[(1S)-1-[4-(4-methylthiazol-5-yl)phenyl]ethyl]carbamoyl]pyrrolidine-1- |
| | carbonyl]-2,2-dimethyl-propyl]amino]-8-oxo-octyl]pyridine-2-carboxamide |
| 130 | 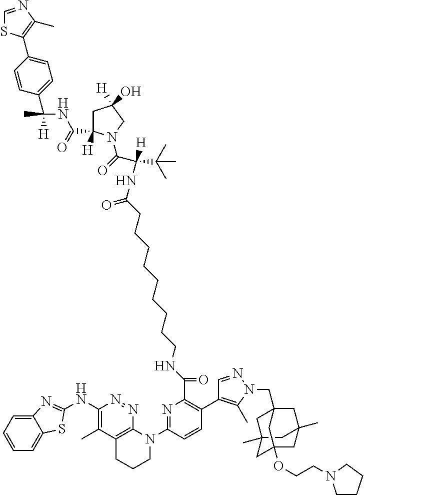 |
| | 6-[3-(1,3-benzothiazol-2-ylamino)-4-methyl-6,7-dihydro-5H-pyrido[2,3- |
| | c]pyridazin-8-yl]-3-[1-[[3,5-dimethyl-7-(2-pyrrolidin-1-ylethoxy)-1- |
| | adamantyl]methyl]-5-methyl-pyrazol-4-yl]-N-[10-[[(1S)-1-[(2S,4R)-4- |
| | hydroxy-2-[[(1S)-1-[4-(4-methylthiazol-5- |
| | yl)phenyl]ethyl]carbamoyl]pyrrolidine-1-carbonyl]-2,2-dimethyl- |
| | propyl]amino]-10-oxo-decyl]pyridine-2-carboxamide |
| 131 | 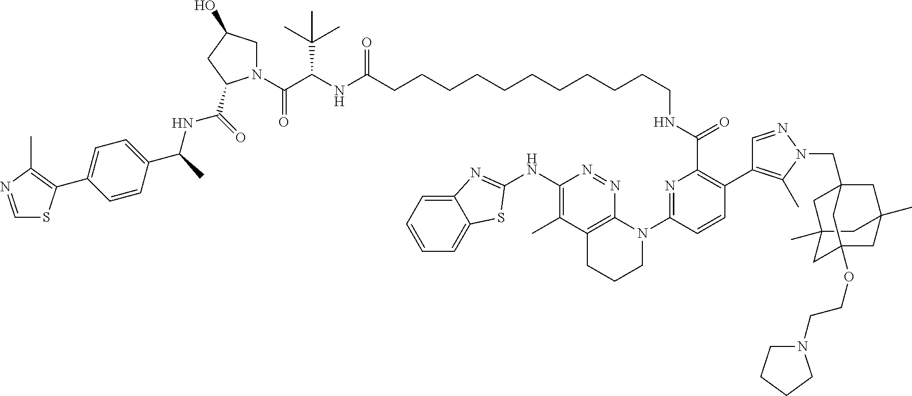 |
| | 6-[3-(1,3-benzothiazol-2-ylamino)-4-methyl-6,7-dihydro-5H-pyrido[2,3- |
| | c]pyridazin-8-yl]-3-[1-[[3,5-dimethyl-7-(2-pyrrolidin-1-ylethoxy)-1- |
| | adamantyl]methyl]-5-methyl-pyrazol-4-yl]-N-[12-[[(1S)-1-[(2S,4R)-4- |
| | hydroxy-2-[[(1S)-1-[4-(4-methylthiazol-5- |
| | yl)phenyl]ethyl]carbamoyl]pyrrolidine-1-carbonyl]-2,2-dimethyl- |
| | propyl]amino]-12-oxo-dodecyl]pyridine-2-carboxamide |
| 132 | 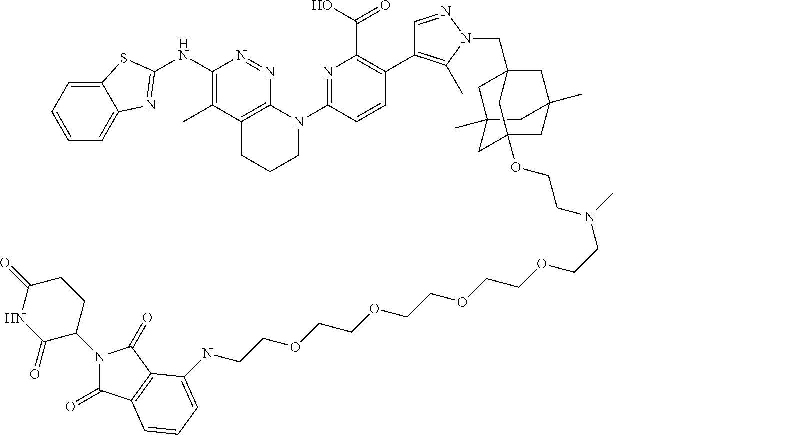 |
| | 6-[3-(1,3-benzothiazol-2-ylamino)-4-methyl-6,7-dihydro-5H-pyrido[2,3- |
| | c]pyridazin-8-yl]-3-[1-[[3-[2-[2-[2-[2-[2-[2-[[2-(2,6-dioxo-3-piperidyl)-1,3- |
| | dioxo-isoindolin-4-yl]amino]ethoxy]ethoxy]ethoxy]ethoxy]ethyl-methyl- |
| | amino]ethoxy]-5,7-dimethyl-1-adamantyl]methyl]-5-methyl-pyrazol-4- |
| | yl]pyridine-2-carboxylic acid |
| 133 | 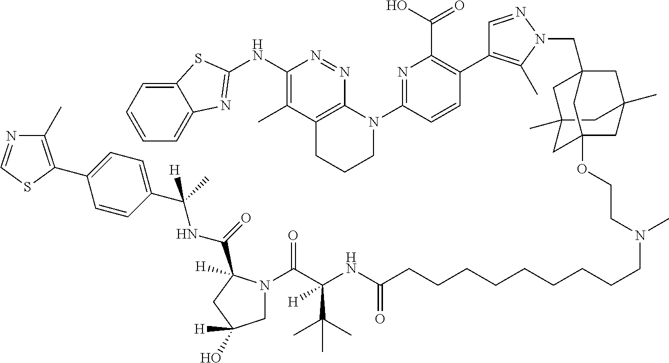 [SMILES: Cc1ncsc1-c1ccc([C@H](C)NC(=O)[C@@H]2C[C@@H](O)CN2C(=O)[C@@H](NC(=O)CCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[[10-[[(1S)-1-[(2S,4R)-4-hydroxy-2-[[(1S)-1-[4- |
| | (4-methylthiazol-5-yl)phenyl]ethyl]carbamoyl]pyrrolidine-1-carbonyl]-2,2- |
| | dimethyl-propyl]amino]-10-oxo-decyl]-methyl-amino]ethoxy]-5,7-dimethyl-1- |
| | adamantyl]methyl]-5-methyl-pyrazol-4-yl]pyridine-2-carboxylic acid |
| 134 | 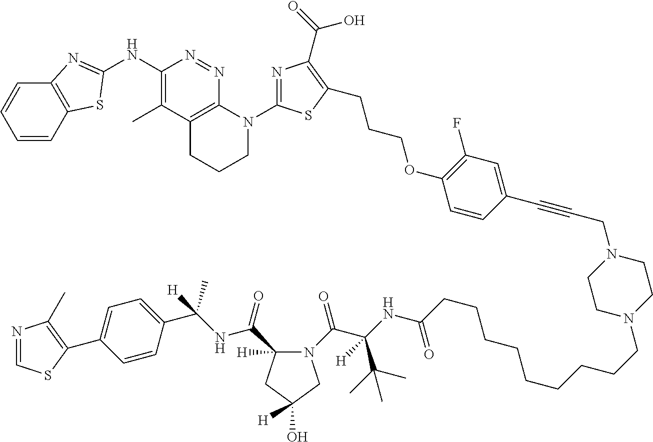 [SMILES: Cc1ncsc1-c1ccc([C@H](C)NC(=O)[C@@H]2C[C@@H](O)CN2C(=O)[C@@H](NC(=O)CCCCCCCCCN2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1] |
| | 2-[3-(1,3-benzothiazol-2-ylamino)-4-methyl-6,7-dihydro-5H-pyrido[2,3- |
| | c]pyridazin-8-yl]-5-[3-[2-fluoro-4-[3-[4-[10-[[(1S)-1-[(2S,4R)-4-hydroxy-2- |
| | [[(1S)-1-[4-(4-methylthiazol-5-yl)phenyl]ethyl]carbamoyl]pyrrolidine-1- |
| | carbonyl]-2,2-dimethyl-propyl]amino]-10-oxo-decyl]piperazin-1-yl]prop-1- |
| | ynyl]phenoxy]propyl]thiazole-4-carboxylic acid |
| 135 | 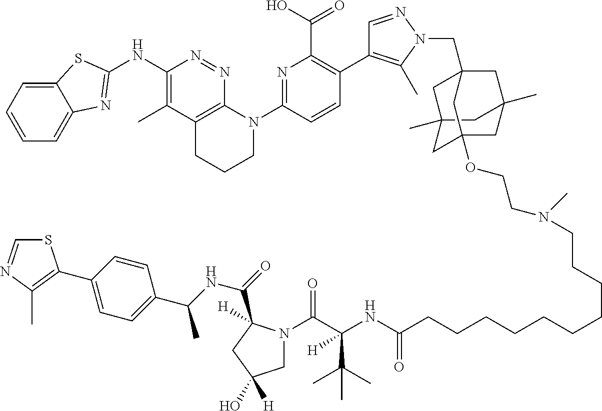 [SMILES: Cc1ncsc1-c1ccc([C@H](C)NC(=O)[C@@H]2C[C@@H](O)CN2C(=O)[C@@H](NC(=O)CCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[[11-[(1S)-1-[(2S,4R)-4-hydroxy-2-[[(1S)-1-[4- |
| | (4-methylthiazol-5-yl)phenyl]ethyl]carbamoyl]pyrrolidine-1-carbonyl]-2,2- |
| | dimethyl-propyl]amino]-11-oxo-undecyl]-methyl-amino]ethoxy]-5,7- |
| | dimethyl-1-adamantyl]methyl]-5-methyl-pyrazol-4-yl]pyridine-2-carboxylic |
| | acid |
| 136 | 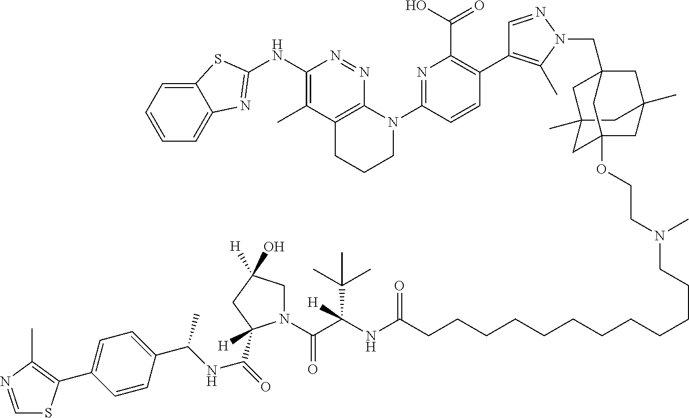 [SMILES: Cc1ncsc1-c1ccc([C@H](C)NC(=O)[C@@H]2C[C@@H](O)CN2C(=O)[C@@H](NC(=O)CCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[[13-[[(1S)-1-[(2S,4R)-4-hydroxy-2-[[(1S)-1-[4- |
| | (4-methylthiazol-5-yl)phenyl]ethyl]carbamoyl]pyrrolidine-1-carbonyl]-2,2- |
| | dimethyl-propyl]amino]-13-oxo-tridecyl]-methyl-amino]ethoxy]-5,7-dimethyl- |
| | 1-adamantyl]methyl]-5-methyl-pyrazol-4-yl]pyridine-2-carboxylic acid |
| 137 | 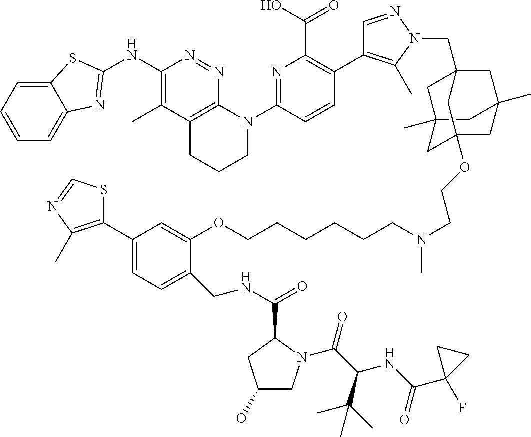 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)c1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[6-[2-[[[(2S,4R)-1-[(2S)-2-[(1- |
| | fluorocyclopropanecarbonyl)amino]-3,3-dimethyl-butanoyl]-4-hydroxy- |
| | pyrrolidine-2-carbonyl]amino]methyl]-5-(4-methylthiazol-5- |
| | yl)phenoxy]hexyl-methyl-amino]ethoxy]-5,7-dimethyl-1-adamantyl]methyl]- |
| | 5-methyl-pyrazol-4-yl]pyridine-2-carboxylic acid |
| 138 | 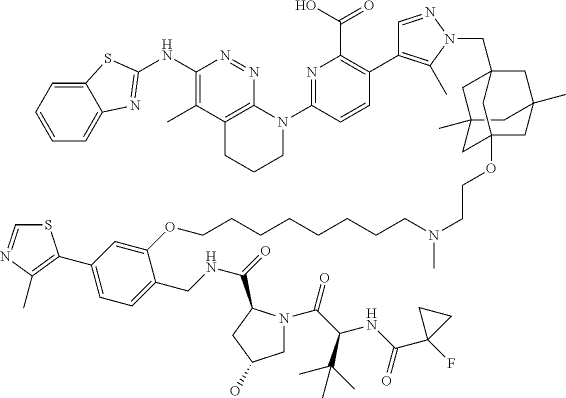 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)c1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[8-[2-[[[(2S,4R)-1-[(2S)-2-[(1- |
| | fluorocyclopropanecarbonyl)amino]-3,3-dimethyl-butanoyl]-4-hydroxy- |
| | pyrrolidine-2-carbonyl]amino]methyl]-5-(4-methylthiazol-5-yl)phenoxy]octyl- |
| | methyl-amino]ethoxy]-5,7-dimethyl-1-adamantyl]methyl]-5-methyl-pyrazol- |
| | 4-yl]pyridine-2-carboxylic acid |
| 139 | 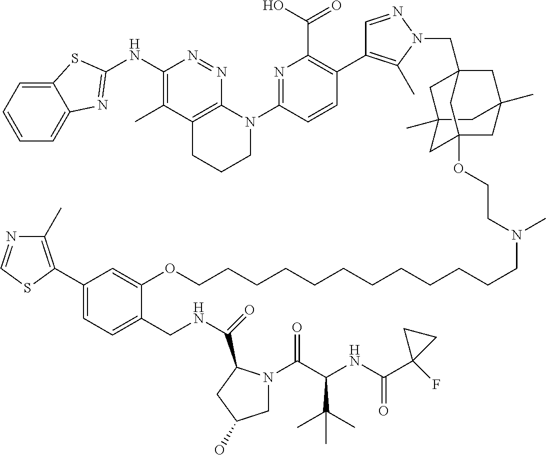 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)c1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[12-[2-[[[(2S,4R)-1-[(2S)-2-[(1- |
| | fluorocyclopropanecarbonyl)amino]-3,3-dimethyl-butanoyl]-4-hydroxy- |
| | pyrrolidine-2-carbonyl]amino]methyl]-5-(4-methylthiazol-5- |
| | yl)phenoxy]dodecyl-methyl-amino]ethoxy]-5,7-dimethyl-1- |
| | adamantyl]methyl]-5-methyl-pyrazol-4-yl]pyridine-2-carboxylic acid |
| 140 | 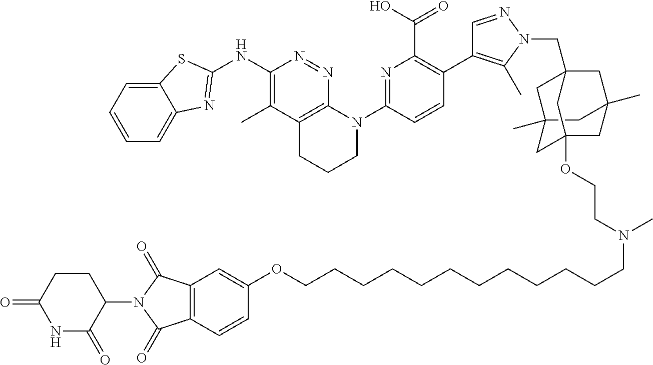 [SMILES: Cc1c(Nc2nc3ccccc3s2)nnc2c1CCCN2c1ccc(-c2cnn(CC34CC5(C)CC(C)(C3)CC(OCCN(C)CCCCCCCCCCCCOc3ccc6c(c3)C(=O)N(C3CCC(=O)NC3=O)C6=O)(C5)C4)c2C)c(C(=O)O)n1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[12-[2-(2,6-dioxo-3-piperidyl)-1,3-dioxo- |
| | isoindolin-5-yl]oxydodecyl-methyl-amino]ethoxy]-5,7-dimethyl-1- |
| | adamantyl]methyl]-5-methyl-pyrazol-4-yl]pyridine-2-carboxylic acid |
| 141 | 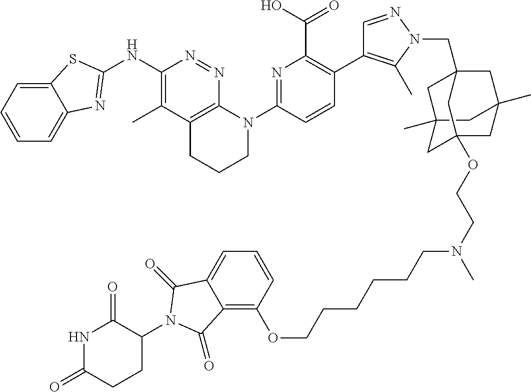 [SMILES: Cc1c(Nc2nc3ccccc3s2)nnc2c1CCCN2c1ccc(-c2cnn(CC34CC5(C)CC(C)(C3)CC(OCCN(C)CCCCCCOc3cccc6c3C(=O)N(C3CCC(=O)NC3=O)C6=O)(C5)C4)c2C)c(C(=O)O)n1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[6-[2-(2,6-dioxo-3-piperidyl)-1,3-dioxo-isoindolin- |
| | 4-yl]oxyhexyl-methyl-amino]ethoxy]-5,7-dimethyl-1-adamantyl]methyl]-5- |
| | methyl-pyrazol-4-yl]pyridine-2-carboxylic acid |
| 142 | 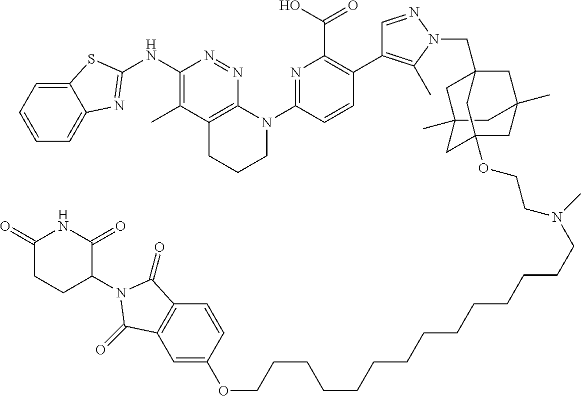 [SMILES: Cc1c(Nc2nc3ccccc3s2)nnc2c1CCCN2c1ccc(-c2cnn(CC34CC5(C)CC(C)(C3)CC(OCCN(C)CCCCCCCCCCCCCCOc3ccc6c(c3)C(=O)N(C3CCC(=O)NC3=O)C6=O)(C5)C4)c2C)c(C(=O)O)n1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[14-[2-(2,6-dioxo-3-piperidyl)-1,3-dioxo- |
| | isoindolin-5-yl]oxytetradecyl-methyl-amino]ethoxy]-5,7-dimethyl-1- |
| | adamantyl]methyl]-5-methyl-pyrazol-4-yl]pyridine-2-carboxylic acid |
| 143 | 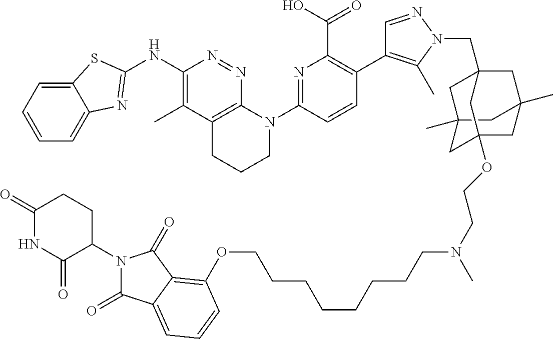 [SMILES: Cc1c(Nc2nc3ccccc3s2)nnc2c1CCCN2c1ccc(-c2cnn(CC34CC5(C)CC(C)(C3)CC(OCCN(C)CCCCCCCCOc3cccc6c3C(=O)N(C3CCC(=O)NC3=O)C6=O)(C5)C4)c2C)c(C(=O)O)n1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[8-[2-(2,6-dioxo-3-piperidyl)-1,3-dioxo-isoindolin- |
| | 4-yl]oxyoctyl-methyl-amino]ethoxy]-5,7-dimethyl-1-adamantyl]methyl]-5- |
| | methyl-pyrazol-4-yl]pyridine-2-carboxylic acid |
| 144 | 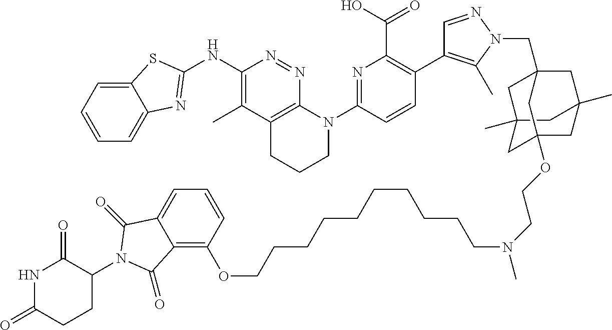 [SMILES: Cc1c(Nc2nc3ccccc3s2)nnc2c1CCCN2c1ccc(-c2cnn(CC34CC5(C)CC(C)(C3)CC(OCCN(C)CCCCCCCCCCOc3cccc6c3C(=O)N(C3CCC(=O)NC3=O)C6=O)(C5)C4)c2C)c(C(=O)O)n1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[10-[2-(2,6-dioxo-3-piperidyl)-1,3-dioxo- |
| | isoindolin-4-yl]oxydecyl-methyl-amino]ethoxy]-5,7-dimethyl-1- |
| | adamantyl]methyl]-5-methyl-pyrazol-4-yl]pyridine-2-carboxylic acid |
| 145 | 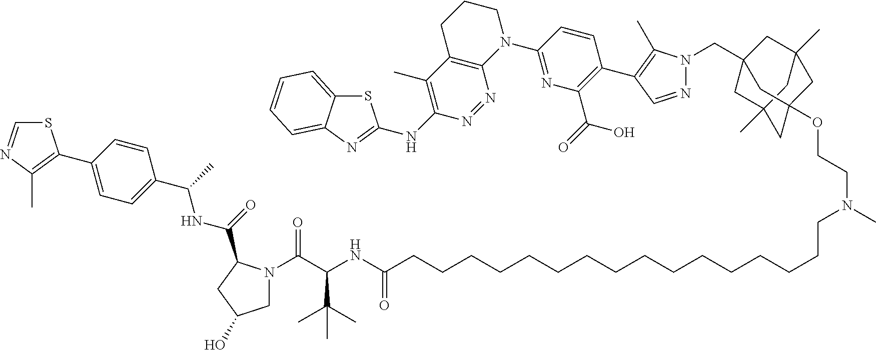 [SMILES: Cc1ncsc1-c1ccc([C@H](C)NC(=O)[C@@H]2C[C@@H](O)CN2C(=O)[C@@H](NC(=O)CCCCCCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[[17-[[(1S)-1-[(2S,4R)-4-hydroxy-2-[[(1S)-1-[4- |
| | (4-methylthiazol-5-yl)phenyl]ethyl]carbamoyl]pyrrolidine-1-carbonyl]-2,2- |
| | dimethyl-propyl]amino]-17-oxo-heptadecyl]-methyl-amino]ethoxy]-5,7- |
| | dimethyl-1-adamantyl]methyl]-5-methyl-pyrazol-4-yl]pyridine-2-carboxylic |
| | acid |
| 146 | 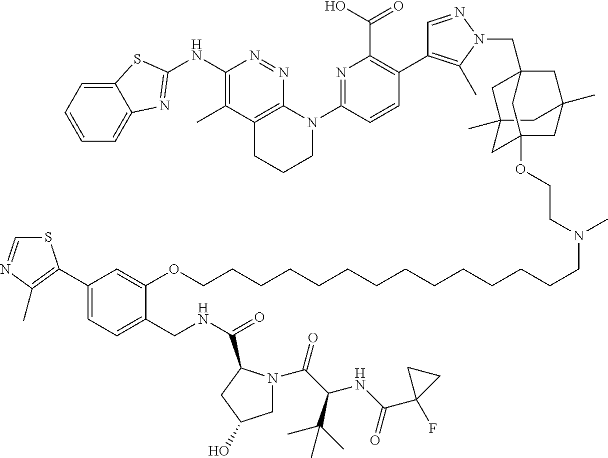 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)c1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[14-[2-[[[(2S,4R)-1-[(2S)-2-[(1- |
| | fluorocyclopropanecarbonyl)amino]-3,3-dimethyl-butanoyl]-4-hydroxy- |
| | pyrrolidine-2-carbonyl]amino]methyl]-5-(4-methylthiazol-5- |
| | yl)phenoxy]tetradecyl-methyl-amino]ethoxy]-5,7-dimethyl-1- |
| | adamantyl]methyl]-5-methyl-pyrazol-4-yl]pyridine-2-carboxylic acid |
| 147 | 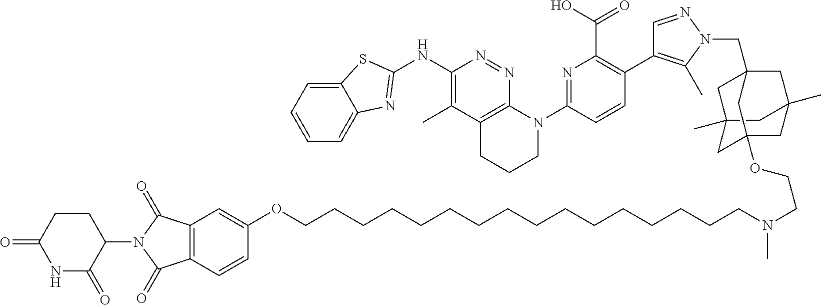 |
| | 6-[3-(1,3-benzothiazol-2-ylamino)-4-methyl-6,7-dihydro-5H-pyrido[2,3- |
| | c]pyridazin-8-yl]-3-[1-[[3-[2-[16-[2-(2,6-dioxo-3-piperidyl)-1,3-dioxo- |
| | isoindolin-5-yl]oxyhexadecyl-methyl-amino]ethoxy]-5,7-dimethyl-1- |
| | adamantyl]methyl]-5-methyl-pyrazol-4-yl]pyridine-2-carboxylic acid |
| 148 | 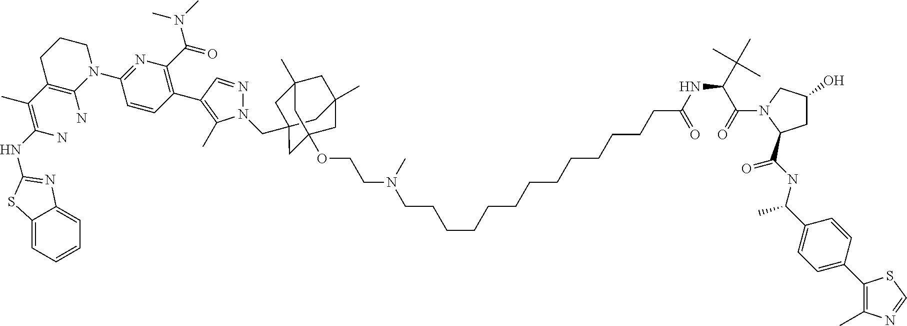 |
| | 6-[3-(1,3-benzothiazol-2-ylamino)-4-methyl-6,7-dihydro-5H-pyrido[2,3- |
| | c]pyridazin-8-yl]-3-[1-[[3-[2-[[14-[[(1S)-1-[(2S,4R)-4-hydroxy-2-[[(1S)-1-[4- |
| | (4-methylthiazol-5-yl)phenyl]ethyl]carbamoyl]pyrrolidine-1-carbonyl]-2,2- |
| | dimethyl-propyl]amino]-14-oxo-tetradecyl]-methyl-amino]ethoxy]-5,7- |
| | dimethyl-1-adamantyl]methyl]-5-methyl-pyrazol-4-yl]-N,N-dimethyl-pyridine- |
| | 2-carboxamide |
| 149 | 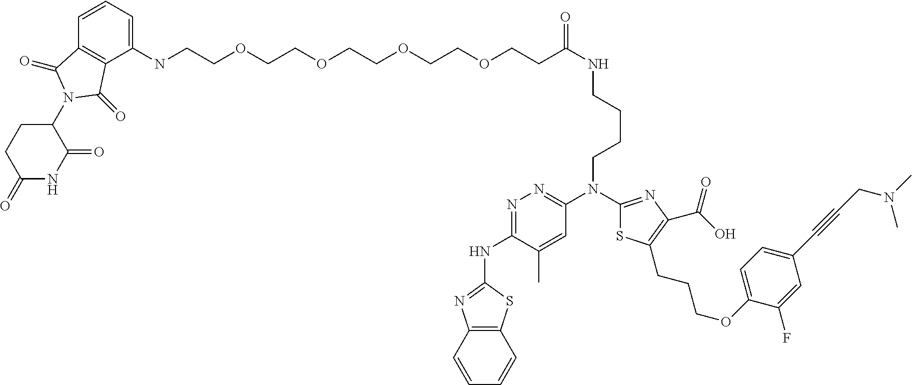 |
| | 2-[[6-(1,3-benzothiazol-2-ylamino)-5-methyl-pyridazin-3-yl]-[4-[3-[2-[2-[2-[2- |
| | [[2-(2,6-dioxo-3-piperidyl)-1,3-dioxo-isoindolin-4- |
| | yl]amino]ethoxy]ethoxy]ethoxy]ethoxy]propanoylamino]butyl]amino]-5-[3-[4- |
| | [3-(dimethylamino)prop-1-ynyl]-2-fluoro-phenoxy]propyl]thiazole-4- |
| | carboxylic acid |
| 150 | 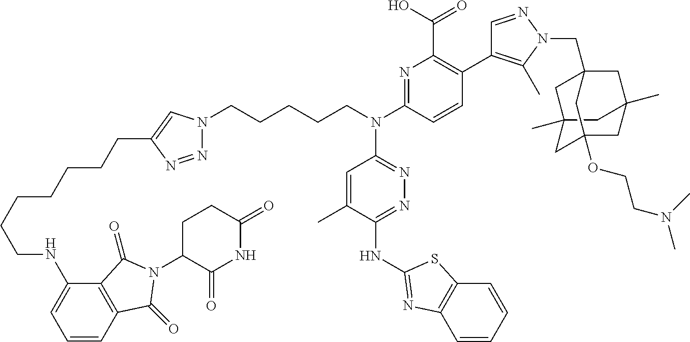 [SMILES: Cc1cc(N(CCCCCn2cc(CCCCCCCNc3cccc4c3C(=O)N(C3CCC(=O)NC3=O)C4=O)nn2)c2ccc(-c3cnn(CC45CC6(C)CC(C)(C4)CC(OCCN(C)C)(C6)C5)c3C)c(C(=O)O)n2)nnc1Nc1nc2ccccc2s1] |
| | 6-[[6-(1,3-benzothiazol-2-ylamino)-5-methyl-pyridazin-3-yl]-[5-[4-[7-[[2-(2,6- |
| | dioxo-3-piperidyl)-1,3-dioxo-isoindolin-4-yl]amino]heptyl]triazol-1- |
| | yl]pentyl]amino]-3-[1-[[3-[2-(dimethylamino)ethoxy]-5,7-dimethyl-1- |
| | adamantyl]methyl]-5-methyl-pyrazol-4-yl]pyridine-2-carboxylic acid |
| 151 | 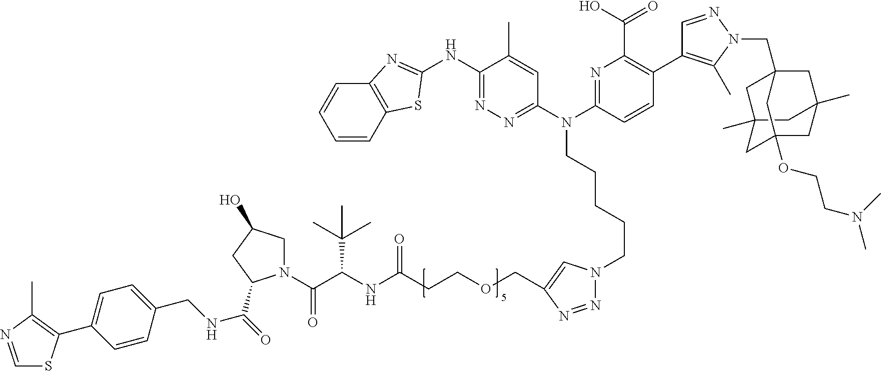 |
| | 6-[[6-(1,3-benzothiazol-2-ylamino)-5-methyl-pyridazin-3-yl]-[5-[4-[[3-[[(1S)- |
| | 1-[(2S,4R)-4-hydroxy-2-[[4-(4-methylthiazol-5- |
| | yl)phenyl]methylcarbamoyl]pyrrolidine-1-carbonyl]-2,2-dimethyl- |
| | propyl]amino]-3-oxo-propoxy]methyl]triazol-1-yl]pentyl]amino]-3-[1-[[3-[2- |
| | (dimethylamino)ethoxy]-5,7-dimethyl-1-adamantyl]methyl]-5-methyl- |
| | pyrazol-4-yl]pyridine-2-carboxylic acid |
| 152 | 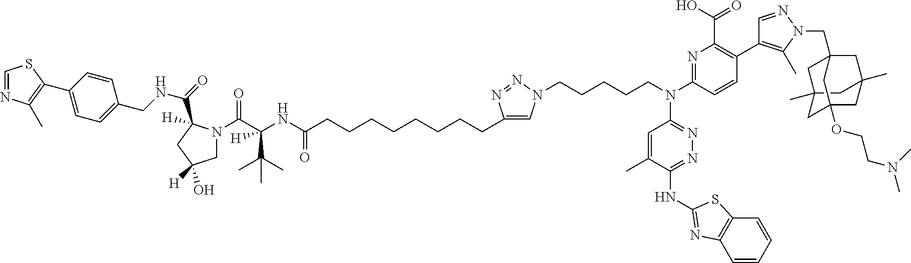 [SMILES: Cc1cc(N(CCCCCn2cc(CCCCCCCCC(=O)N[C@H](C(=O)N3C[C@H](O)C[C@H]3C(=O)NCc3ccc(-c4scnc4C)cc3)C(C)(C)C)nn2)c2ccc(-c3cnn(CC45CC6(C)CC(C)(C4)CC(OCCN(C)C)(C6)C5)c3C)c(C(=O)O)n2)nnc1Nc1nc2ccccc2s1] |
| | 6-[[6-(1,3-benzothiazol-2-ylamino)-5-methyl-pyridazin-3-yl]-[5-[4-[9-[[(1S)-1- |
| | [(2S,4R)-4-hydroxy-2-[[4-(4-methylthiazol-5- |
| | yl)phenyl]methylcarbamoyl]pyrrolidine-1-carbonyl]-2,2-dimethyl- |
| | propyl]amino]-9-oxo-nonyl]triazol-1-yl]pentyl]amino]-3-[1-[[3-[2- |
| | (dimethylamino)ethoxy]-5,7-dimethyl-1-adamantyl]methyl]-5-methyl- |
| | pyrazol-4-yl]pyridine-2-carboxylic acid |
| 153 | 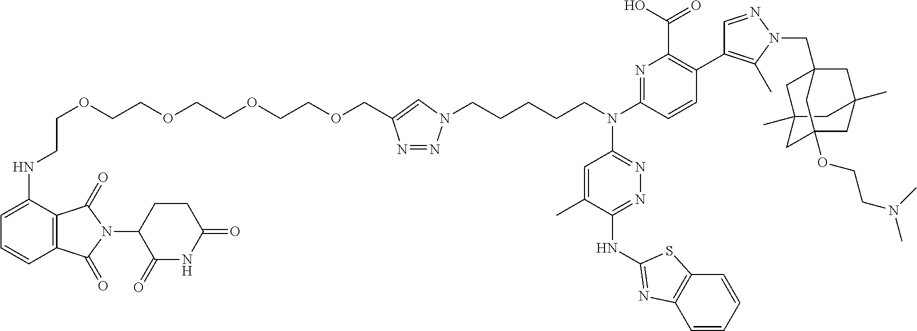 [SMILES: Cc1cc(N(CCCCCn2cc(COCCOCCOCCOCCNc3cccc4c3C(=O)N(C3CCC(=O)NC3=O)C4=O)nn2)c2ccc(-c3cnn(CC45CC6(C)CC(C)(C4)CC(OCCN(C)C)(C6)C5)c3C)c(C(=O)O)n2)nnc1Nc1nc2ccccc2s1] |
| | 6-[[6-(1,3-benzothiazol-2-ylamino)-5-methyl-pyridazin-3-yl]-[5-[4-[2-[2-[2-[2- |
| | [[2-(2,6-dioxo-3-piperidyl)-1,3-dioxo-isoindolin-4- |
| | yl]amino]ethoxy]ethoxy]ethoxy]ethoxymethyl]triazol-1-yl]pentyl]amino]-3-[1- |
| | [[3-[2-(dimethylamino)ethoxy]-5,7-dimethyl-1-adamantyl]methyl]-5-methyl- |
| | pyrazol-4-yl]pyridine-2-carboxylic acid |
| 154 | 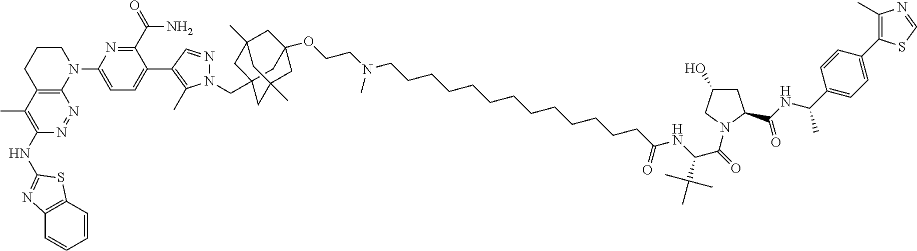 [SMILES: Cc1ncsc1-c1ccc([C@H](C)NC(=O)[C@@H]2C[C@@H](O)CN2C(=O)[C@@H](NC(=O)CCCCCCCCCCCCCN(C)CCOC23CC4(C)CC(C)(CC(Cn5ncc(-c6ccc(N7CCCc8c7nnc(Nc7nc9ccccc9s7)c8C)nc6C(N)=O)c5C)(C4)C2)C3)C(C)(C)C)cc1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[[14-[[(1S)-1-[(2S,4R)-4-hydroxy-2-[[(1S)-1-[4- |
| | (4-methylthiazol-5-yl)phenyl]ethyl]carbamoyl]pyrrolidine-1-carbonyl]-2,2- |
| | dimethyl-propyl]amino]-14-oxo-tetradecyl]-methyl-amino]ethoxy]-5,7- |
| | dimethyl-1-adamantyl]methyl]-5-methyl-pyrazol-4-yl]pyridine-2- |
| | carboxamide |
| 155 | 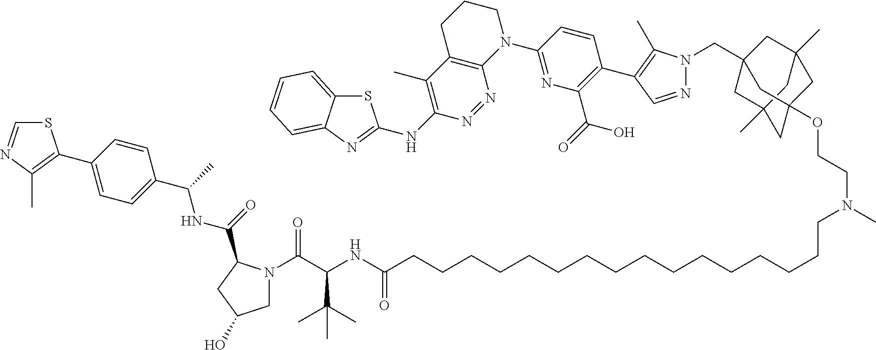 [SMILES: Cc1ncsc1-c1ccc([C@H](C)NC(=O)[C@@H]2C[C@@H](O)CN2C(=O)[C@@H](NC(=O)CCCCCCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1] |
| | 2S,4R)-1-[(2S)-2-[14-[2-[[3-[4-[6-[3-(1,3-benzothiazol-2-ylamino)-4-methyl- |
| | 6,7-dihydro-5H-pyrido[2,3-c]pyridazin-8-yl]-2-(hydroxymethyl)-3-pyridyl]-5- |
| | methyl-pyrazol-1-yl]methyl]-5,7-dimethyl-1-adamantyl]oxy]ethyl-methyl- |
| | amino]tetradecanoylamino]-3,3-dimethyl-butanoyl]-4-hydroxy-N-[(1S)-1-[4- |
| | (4-methylthiazol-5-yl)phenyl]ethyl]pyrrolidine-2-carboxamide |
| 156 | 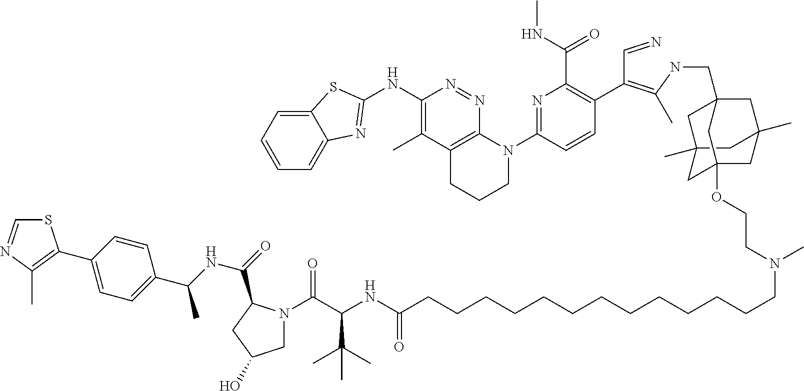 [SMILES: CNC(=O)c1nc(N2CCCc3c2nnc(Nc2nc4ccccc4s2)c3C)ccc1/C(C=N)=C(\C)NCC12CC3(C)CC(C)(C1)CC(OCCN(C)CCCCCCCCCCCCCC(=O)N[C@H](C(=O)N1C[C@H](O)C[C@H]1C(=O)N[C@@H](C)c1ccc(-c4scnc4C)cc1)C(C)(C)C)(C3)C2] |
| | 6-[3-(1,3-benzothiazol-2-ylamino)-4-methyl-6,7-dihydro-5H-pyrido[2,3- |
| | c]pyridazin-8-yl]-3-[1-[[3-[2-[[14-[[(1S)-1-[(2S,4R)-4-hydroxy-2-[[(1S)-1-[4- |
| | (4-methylthiazol-5-yl)phenyl]ethyl]carbamoyl]pyrrolidine-1-carbonyl]-2,2- |
| | dimethyl-propyl]amino]-14-oxo-tetradecyl]-methyl-amino]ethoxy]-5,7- |
| | dimethyl-1-adamantyl]methyl]-5-methyl-pyrazol-4-yl]-N-methyl-pyridine-2- |
| | carboxamide |
| 157 | 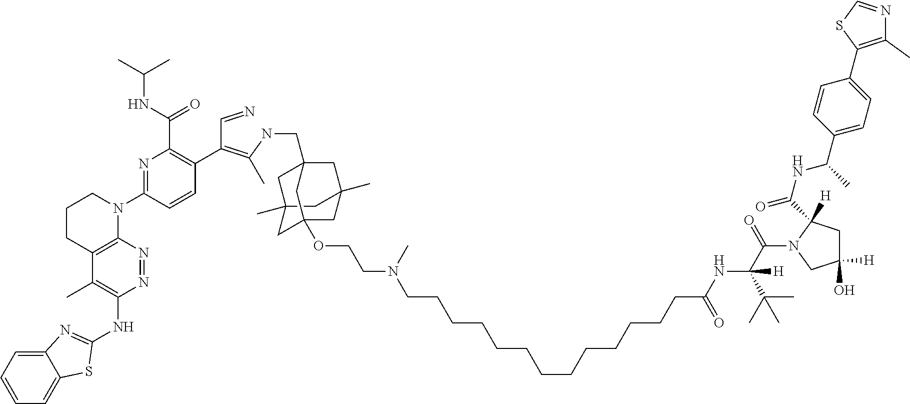 [SMILES: C/C(NCC12CC3(C)CC(C)(C1)CC(OCCN(C)CCCCCCCCCCCCCC(=O)N[C@H](C(=O)N1C[C@H](O)C[C@H]1C(=O)N[C@@H](C)c1ccc(-c4scnc4C)cc1)C(C)(C)C)(C3)C2)=C(/C=N)c1ccc(N2CCCc3c2nnc(Nc2nc4ccccc4s2)c3C)nc1C(=O)NC(C)C] |
| | 6-[3-(1,3-benzothiazol-2-ylamino)-4-methyl-6,7-dihydro-5H-pyrido[2,3- |
| | c]pyridazin-8-yl]-3-[1-[[3-[2-[[14-[[(1S)-1-[(2S,4R)-4-hydroxy-2-[[(1S)-1-[4- |
| | (4-methylthiazol-5-yl)phenyl]ethyl]carbamoyl]pyrrolidine-1-carbonyl]-2,2- |
| | dimethyl-propyl]amino]-14-oxo-tetradecyl]-methyl-amino]ethoxy]-5,7- |
| | dimethyl-1-adamantyl]methyl]-5-methyl-pyrazol-4-yl]-N-isopropyl-pyridine- |
| | 2-carboxamide |
| 158 | 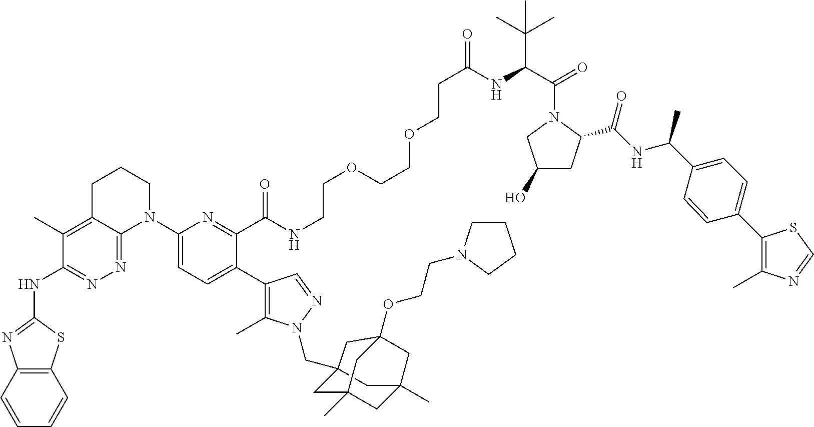 [SMILES: Cc1ncsc1-c1ccc([C@H](C)NC(=O)[C@@H]2C[C@@H](O)CN2C(=O)[C@@H](NC(=O)CCOCCOCCNC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C(C)(C)C)cc1] |
| | 6-(3-(benzo[d]thiazol-2-ylamino)-4-methyl-6,7-dihydropyrido[2,3-c] |
| | pyridazin-8(5H)-yl)-3-(1-(((1r,3R,5S,7s)-3,5-dimethyl-7-(2-(pyrrolidin-1- |
| | yl)ethoxy)adamantan-1-yl)methyl)-5-methyl-1H-pyrazol-4-yl)-N-(2-(2-(3- |
| | (((S)-1-((2S,4R)-4-hydroxy-2-(((S)-1-(4-(4-methylthiazol-5- |
| | yl)phenyl)ethyl)carbamoyl)pyrrolidin-1-yl)-3,3-dimethyl-1-oxobutan-2- |
| | yl)amino)-3-oxopropoxy)ethoxy)ethyl)picolinamide |
| 159 | 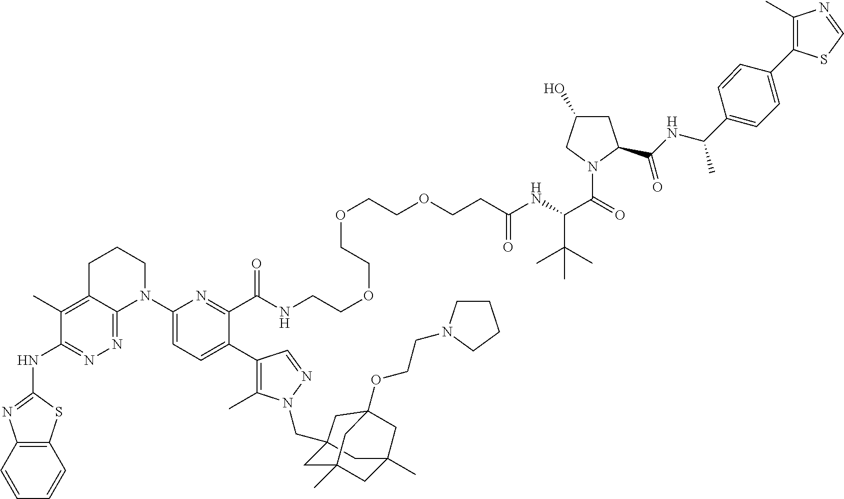 [SMILES: Cc1ncsc1-c1ccc([C@H](C)NC(=O)[C@@H]2C[C@@H](O)CN2C(=O)[C@@H](NC(=O)CCOCCOCCOCCNC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C(C)(C)C)cc1] |
| | 6-(3-(benzo[d]thiazol-2-ylamino)-4-methyl-6,7-dihydropyrido[2,3-c] |
| | pyridazin-8(5H)-yl)-3-(1-(((1r,3R,5S,7s)-3,5-dimethyl-7-(2-(pyrrolidin-1- |
| | yl)ethoxy)adamantan-1-yl)methyl)-5-methyl-1H-pyrazol-4-yl)-N-((S)-14- |
| | ((2S,4R)-4-hydroxy-2-(((S)-1-(4-(4-methyl thiazol-5- |
| | yl)phenyl)ethyl)carbamoyl)pyrrolidine-1-carbonyl)-15,15-dimethyl-12-oxo- |
| | 3,6,9-trioxa-13-azahexadecyl)picolinamide |
| 160 | 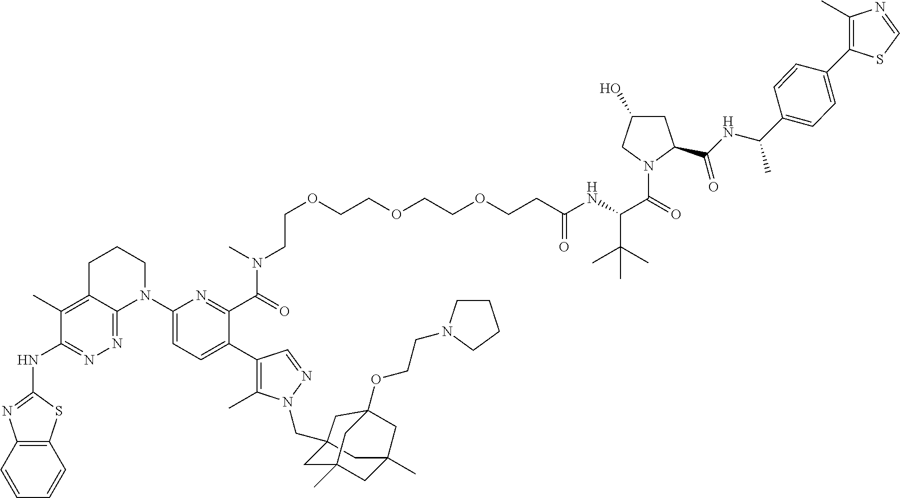 [SMILES: Cc1ncsc1-c1ccc([C@H](C)NC(=O)[C@@H]2C[C@@H](O)CN2C(=O)[C@@H](NC(=O)CCOCCOCCOCCN(C)C(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C(C)(C)C)cc1] |
| | 6-(3-(benzo[d]thiazol-2-ylamino)-4-methyl-6,7-dihydropyrido[2,3-c] |
| | pyridazin-8(5H)-yl)-3-(1-(((1r,3R,5S,7s)-3,5-dimethyl-7-(2-(pyrrolidin-1- |
| | yl)ethoxy)adamantan-1-yl)methyl)-5-methyl-1H-pyrazol-4-yl)-N-((S)-14- |
| | ((2S,4R)-4-hydroxy-2-(((S)-1-(4-(4-methyl thiazol-5- |
| | yl)phenyl)ethyl)carbamoyl)pyrrolidine-1-carbonyl)-15,15-dimethyl-12-oxo- |
| | 3,6,9-trioxa-13-azahexadecyl)-N-methylpicolinamide |
| 161 | 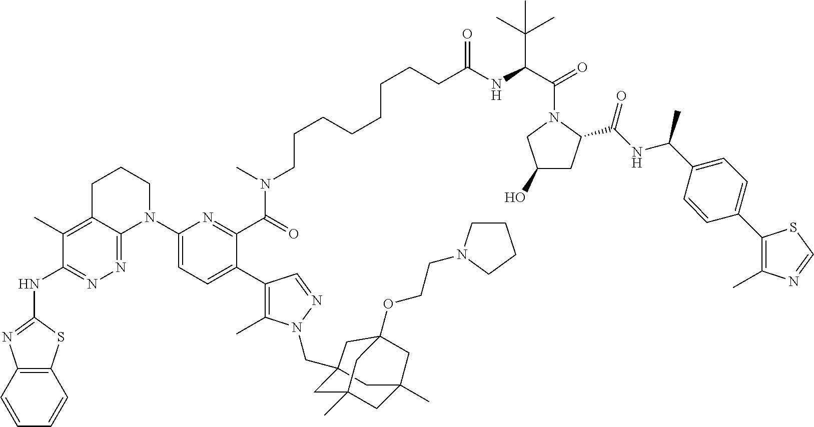 [SMILES: Cc1ncsc1-c1ccc([C@H](C)NC(=O)[C@@H]2C[C@@H](O)CN2C(=O)[C@@H](NC(=O)CCCCCCCCN(C)C(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C(C)(C)C)cc1] |
| | 6-(3-(benzo[d]thiazol-2-ylamino)-4-methyl-6,7-dihydropyrido[2,3-c] |
| | pyridazin-8(5H)-yl)-3-(1-(((1r,3R,5S,7s)-3,5-dimethyl-7-(2-(pyrrolidin-1- |
| | yl)ethoxy)adamantan-1-yl)methyl)-5-methyl-1H-pyrazol-4-yl)-N-(9-(((S)-1- |
| | ((2S,4R)-4-hydroxy-2-(((S)-1-(4-(4-methyl thiazol-5- |
| | yl)phenyl)ethyl)carbamoyl)pyrrolidin-1-yl)-3,3-dimethyl-1-oxobutan-2- |
| | yl)amino)-9-oxononyl)-N-methylpicolinamide |
| 162 | 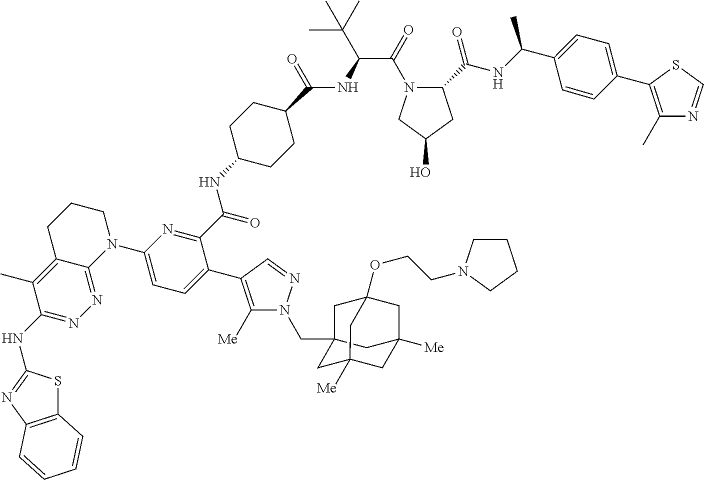 [SMILES: Cc1ncsc1-c1ccc([C@H](C)NC(=O)[C@@H]2C[C@@H](O)CN2C(=O)[C@@H](NC(=O)[C@H]2CC[C@H](NC(=O)c3nc(N4CCCc5c4nnc(Nc4nc6ccccc6s4)c5C)ccc3-c3cnn(CC45CC6(C)CC(C)(C4)CC(OCCN4CCCC4)(C6)C5)c3C)CC2)C(C)(C)C)cc1] |
| | 6-(3-(benzo[d]thiazol-2-ylamino)-4-methyl-6,7-dihydropyrido[2,3-c] |
| | pyridazin-8(5H)-yl)-3-(1-(((1r,3R,5S,7s)-3,5-dimethyl-7-(2-(pyrrolidin-1- |
| | yl)ethoxy)adamantan-1-yl)methyl)-5-methyl-1H-pyrazol-4-yl)-N-((1S,4R)-4- |
| | (((S)-1-((2S,4R)-4-hydroxy-2-(((S)-1-(4-(4-methylthiazol-5- |
| | yl)phenyl)ethyl)carbamoyl)pyrrolidin-1-yl)-3,3-dimethyl-1-oxobutan-2- |
| | yl)carbamoyl)cyclohexyl)picolinamide |
| 163 | 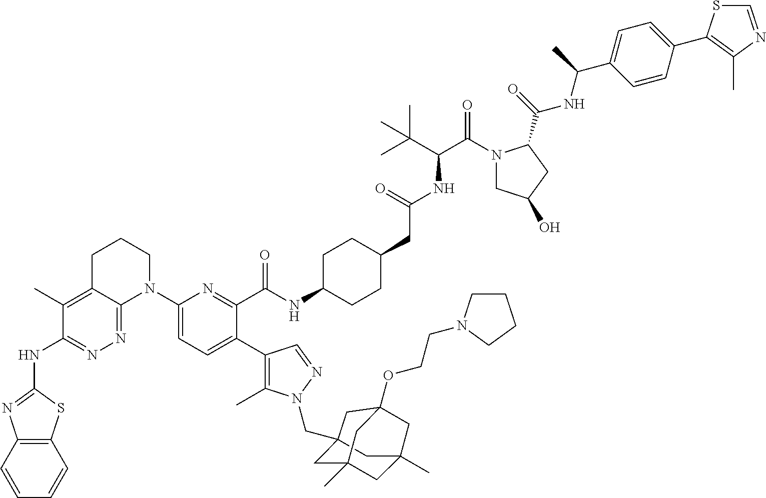 [SMILES: Cc1ncsc1-c1ccc([C@H](C)NC(=O)[C@@H]2C[C@@H](O)CN2C(=O)[C@@H](NC(=O)C[C@H]2CC[C@@H](NC(=O)c3nc(N4CCCc5c4nnc(Nc4nc6ccccc6s4)c5C)ccc3-c3cnn(CC45CC6(C)CC(C)(C4)CC(OCCN4CCCC4)(C6)C5)c3C)CC2)C(C)(C)C)cc1] |
| | 6-(3-(benzo[d]thiazol-2-ylamino)-4-methyl-6,7-dihydropyrido[2,3-c] |
| | pyridazin-8(5H)-yl)-3-(1-(((1r,3R,5S,7s)-3,5-dimethyl-7-(2-(pyrrolidin-1- |
| | yl)ethoxy)adamantan-1-yl)methyl)-5-methyl-1H-pyrazol-4-yl)-N-((1R,4S)-4- |
| | (2-(((S)-1-((2S,4R)-4-hydroxy-2-(((S)-1-(4-(4-methylthiazol-5- |
| | yl)phenyl)ethyl)carbamoyl)pyrrolidin-1-yl)-3,3-dimethyl-1-oxobutan-2- |
| | yl)amino)-2-oxoethyl)cyclohexyl)picolinamide |
| 164 | 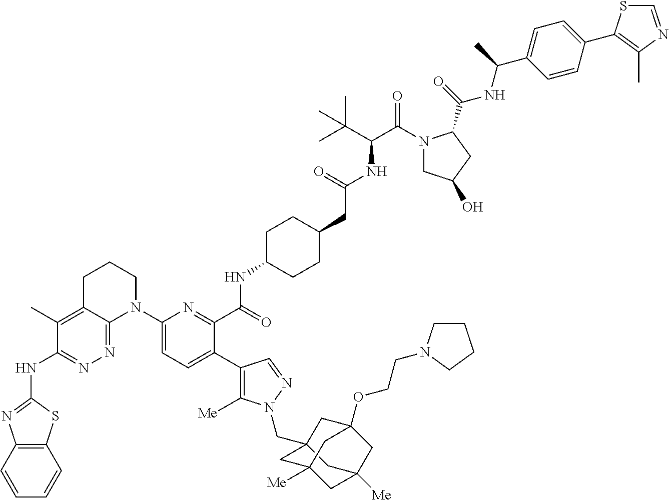 [SMILES: Cc1ncsc1-c1ccc([C@H](C)NC(=O)[C@@H]2C[C@@H](O)CN2C(=O)[C@@H](NC(=O)C[C@H]2CC[C@H](NC(=O)c3nc(N4CCCc5c4nnc(Nc4nc6ccccc6s4)c5C)ccc3-c3cnn(CC45CC6(C)CC(C)(C4)CC(OCCN4CCCC4)(C6)C5)c3C)CC2)C(C)(C)C)cc1] |
| | 6-(3-(benzo[d]thiazol-2-ylamino)-4-methyl-6,7-dihydropyrido[2,3-c] |
| | pyridazin-8(5H)-yl)-3-(1-(((1r,3R,5S,7s)-3,5-dimethyl-7-(2-(pyrrolidin-1- |
| | yl)ethoxy)adamantan-1-yl)methyl)-5-methyl-1 H-pyrazol-4-yl)-N-((1S,4R)-4- |
| | (2-(((S)-1-((2S,4R)-4-hydroxy-2-(((S)-1-(4-(4-methylthiazol-5- |
| | yl)phenyl)ethyl)carbamoyl)pyrrolidin-1-yl)-3,3-dimethyl-1-oxobutan-2- |
| | yl)amino)-2-oxoethyl)cyclohexyl)picolinamide |
| 165 | 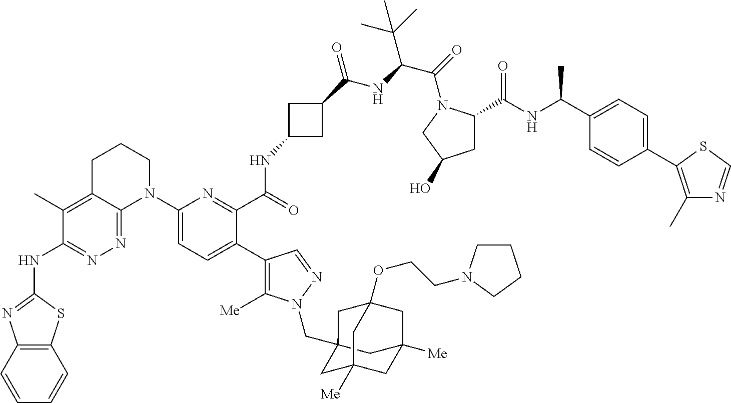 [SMILES: Cc1ncsc1-c1ccc([C@H](C)NC(=O)[C@@H]2C[C@@H](O)CN2C(=O)[C@@H](NC(=O)[C@H]2C[C@H](NC(=O)c3nc(N4CCCc5c4nnc(Nc4nc6ccccc6s4)c5C)ccc3-c3cnn(CC45CC6(C)CC(C)(C4)CC(OCCN4CCCC4)(C6)C5)c3C)C2)C(C)(C)C)cc1] |
| | 6-(3-(benzo[d]thiazol-2-ylamino)-4-methyl-6,7-dihydropyrido[2,3-c] |
| | pyridazin-8(5H)-yl)-3-(1-(((1r,3R,5S,7s)-3,5-dimethyl-7-(2-(pyrrolidin-1- |
| | yl)ethoxy)adamantan-1-yl)methyl)-5-methyl-1H-pyrazol-4-yl)-N-((1S,3R)-3- |
| | (((S)-1-((2S,4R)-4-hydroxy-2-(((S)-1-(4-(4-methylthiazol-5- |
| | yl)phenyl)ethyl)carbamoyl)pyrrolidin-1-yl)-3,3-dimethyl-1-oxobutan-2- |
| | yl)carbamoyl)cyclobutyl)picolinamide |
| 166 | 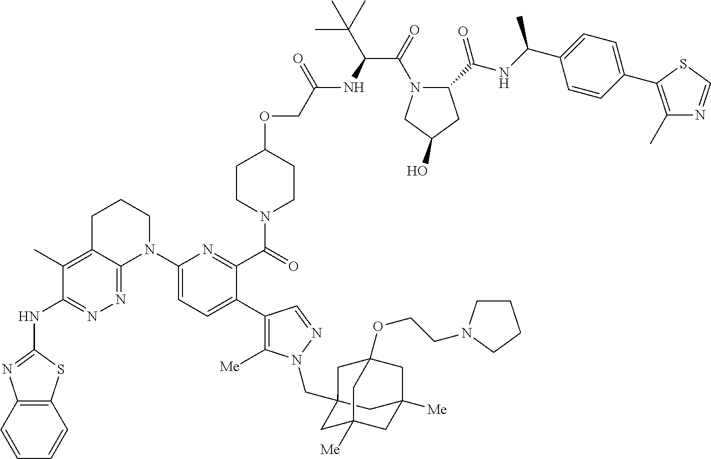 [SMILES: Cc1ncsc1-c1ccc([C@H](C)NC(=O)[C@@H]2C[C@@H](O)CN2C(=O)[C@@H](NC(=O)COC2CCN(C(=O)c3nc(N4CCCc5c4nnc(Nc4nc6ccccc6s4)c5C)ccc3-c3cnn(CC45CC6(C)CC(C)(C4)CC(OCCN4CCCC4)(C6)C5)c3C)CC2)C(C)(C)C)cc1] |
| | (2S,4R)-1-((S)-2-(2-((1-(6-(3-(benzo[d]thiazol-2-ylamino)-4-methyl-6,7- |
| | dihydropyrido[2,3-c]pyridazin-8(5H)-yl)-3-(1-(((1r,3R,5S,7s)-3,5-dimethyl-7- |
| | (2-(pyrrolidin-1-yl)ethoxy)adamantan-1-yl)methyl)-5-methyl-1H-pyrazol-4- |
| | yl)picolinoyl)piperidin-4-yl)oxy)acetamido)-3,3-dimethylbutanoyl)-4- |
| | hydroxy-N-((S)-1-(4-(4-methylthiazol-5-yl)phenyl)ethyl)pyrrolidine-2- |
| | carboxamide |
| 167 | 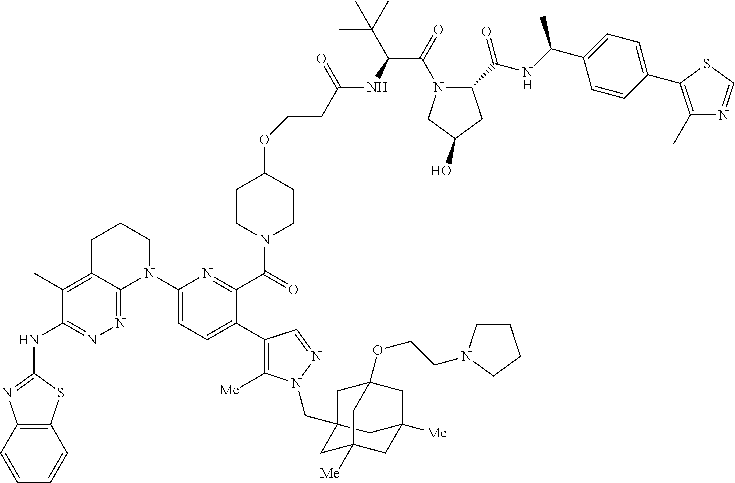 [SMILES: Cc1ncsc1-c1ccc([C@H](C)NC(=O)[C@@H]2C[C@@H](O)CN2C(=O)[C@@H](NC(=O)CCOC2CCN(C(=O)c3nc(N4CCCc5c4nnc(Nc4nc6ccccc6s4)c5C)ccc3-c3cnn(CC45CC6(C)CC(C)(C4)CC(OCCN4CCCC4)(C6)C5)c3C)CC2)C(C)(C)C)cc1] |
| | (2S,4R)-1-((S)-2-(3-((1-(6-(3-(benzo[d]thiazol-2-ylamino)-4-methyl-6,7- |
| | dihydropyrido[2,3-c]pyridazin-8(5H)-yl)-3-(1-(((1r,3R,5S,7s)-3,5-dimethyl-7- |
| | (2-(pyrrolidin-1-yl)ethoxy)adamantan-1-yl)methyl)-5-methyl-1H-pyrazol-4- |
| | yl)picolinoyl)piperidin-4-yl)oxy)propanamido)-3,3-dimethylbutanoyl)-4- |
| | hydroxy-N-((S)-1-(4-(4-methylthiazol-5-yl)phenyl)ethyl)pyrrolidine-2- |
| | carboxamide |
| 168 | 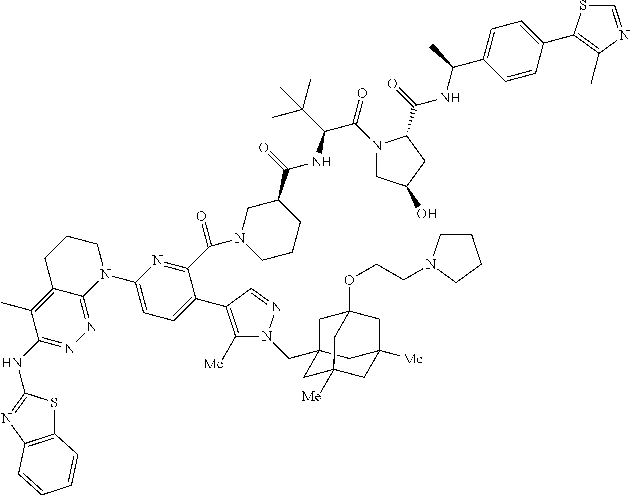 [SMILES: Cc1ncsc1-c1ccc([C@H](C)NC(=O)[C@@H]2C[C@@H](O)CN2C(=O)[C@@H](NC(=O)[C@H]2CCCN(C(=O)c3nc(N4CCCc5c4nnc(Nc4nc6ccccc6s4)c5C)ccc3-c3cnn(CC45CC6(C)CC(C)(C4)CC(OCCN4CCCC4)(C6)C5)c3C)C2)C(C)(C)C)cc1] |
| | (S)-1-(6-(3-(benzo[d]thiazol-2-ylamino)-4-methyl-6,7-dihydropyrido[2,3-c] |
| | pyridazin-8(5H)-yl)-3-(1-(((1r,3R,5S,7R)-3,5-dimethyl-7-(2-(pyrrolidin-1- |
| | yl)ethoxy)adamantan-1-yl)methyl)-5-methyl-1H-pyrazol-4-yl)picolinoyl)-N- |
| | ((S)-1-((2S,4R)-4-hydroxy-2-(((S)-1-(4-(4-methylthiazol-5- |
| | yl)phenyl)ethyl)carbamoyl)pyrrolidin-1-yl)-3,3-dimethyl-1-oxobutan-2- |
| | yl)piperidine-3-carboxamide |
| 169 | 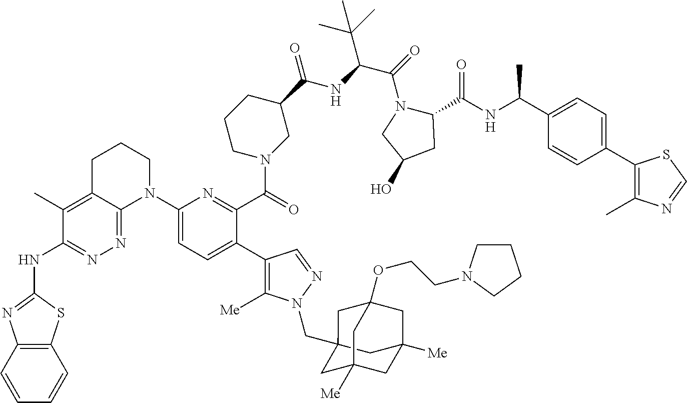 [SMILES: Cc1ncsc1-c1ccc([C@H](C)NC(=O)[C@@H]2C[C@@H](O)CN2C(=O)[C@@H](NC(=O)[C@@H]2CCCN(C(=O)c3nc(N4CCCc5c4nnc(Nc4nc6ccccc6s4)c5C)ccc3-c3cnn(CC45CC6(C)CC(C)(C4)CC(OCCN4CCCC4)(C6)C5)c3C)C2)C(C)(C)C)cc1] |
| | (R)-1-(6-(3-(benzo[d]thiazol-2-ylamino)-4-methyl-6,7-dihydropyrido[2,3-c] |
| | pyridazin-8(5H)-yl)-3-(1-(((1r,3R,5S,7S)-3,5-dimethyl-7-(2-(pyrrolidin-1- |
| | yl)ethoxy)adamantan-1-yl)methyl)-5-methyl-1H-pyrazol-4-yl)picolinoyl)-N- |
| | ((S)-1-((2S,4R)-4-hydroxy-2-(((S)-1-(4-(4-methylthiazol-5- |
| | yl)phenyl)ethyl)carbamoyl)pyrrolidin-1-yl)-3,3-dimethyl-1-oxobutan-2- |
| | yl)piperidine-3-carboxamide |
| 170 | 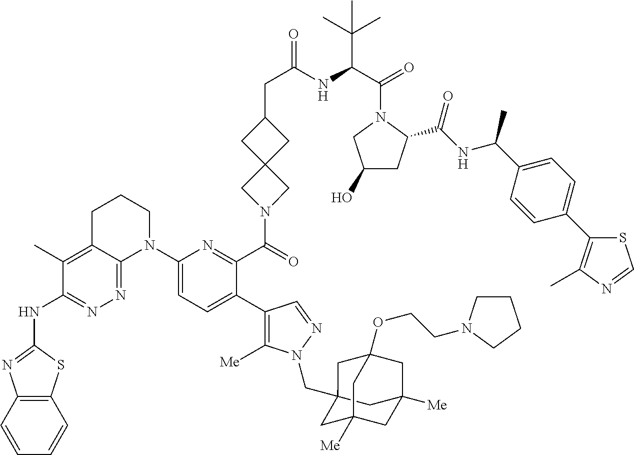 [SMILES: Cc1ncsc1-c1ccc([C@H](C)NC(=O)[C@@H]2C[C@@H](O)CN2C(=O)[C@@H](NC(=O)CC2CC3(C2)CN(C(=O)c2nc(N4CCCc5c4nnc(Nc4nc6ccccc6s4)c5C)ccc2-c2cnn(CC45CC6(C)CC(C)(C4)CC(OCCN4CCCC4)(C6)C5)c2C)C3)C(C)(C)C)cc1] |
| | (2S,4R)-1-((S)-2-(2-(2-(6-(3-(benzo[d]thiazol-2-ylamino)-4-methyl-6,7- |
| | dihydro pyrido[2,3-c]pyridazin-8(5H)-yl)-3-(1-(((1r,3R,5S,7s)-3,5-dimethyl-7- |
| | (2-(pyrrolidin-1-yl)ethoxy)adamantan-1-yl)methyl)-5-methyl-1H-pyrazol-4- |
| | yl)picolinoyl)-2-azaspiro[3.3]heptan-6-yl)acetamido)-3,3-dimethylbutanoyl)- |
| | 4-hydroxy-N-((S)-1-(4-(4-methylthiazol-5-yl)phenyl)ethyl)pyrrolidine-2- |
| | carboxamide |
| 171 | 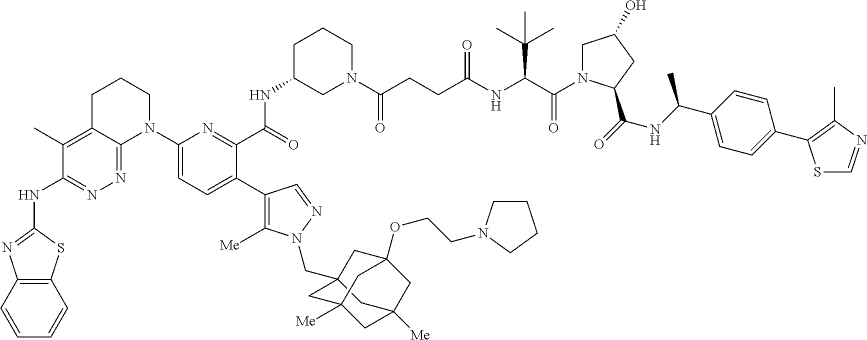 [SMILES: Cc1ncsc1-c1ccc([C@H](C)NC(=O)[C@@H]2C[C@@H](O)CN2C(=O)[C@@H](NC(=O)CCC(=O)N2CCC[C@@H](NC(=O)c3nc(N4CCCc5c4nnc(Nc4nc6ccccc6s4)c5C)ccc3-c3cnn(CC45CC6(C)CC(C)(C4)CC(OCCN4CCCC4)(C6)C5)c3C)C2)C(C)(C)C)cc1] N |
| | 6-(3-(benzo[d]thiazol-2-ylamino)-4-methyl-6,7-dihydropyrido[2,3-c] |
| | pyridazin-8(5H)-yl)-3-(1-(((1r,3R,5S,7S)-3,5-dimethyl-7-(2-(pyrrolidin-1- |
| | yl)ethoxy)adamantan-1-yl)methyl)-5-methyl-1H-pyrazol-4-yl)-N-((R)-1-(4- |
| | (((S)-1-((2S,4R)-4-hydroxy-2-(((S)-1-(4-(4-methylthiazol-5- |
| | yl)phenyl)ethyl)carbamoyl)pyrrolidin-1-yl)-3,3-dimethyl-1-oxobutan-2- |
| | yl)amino)-4-oxobutanoyl)piperidin-3-yl)picolinamide |
| 172 | 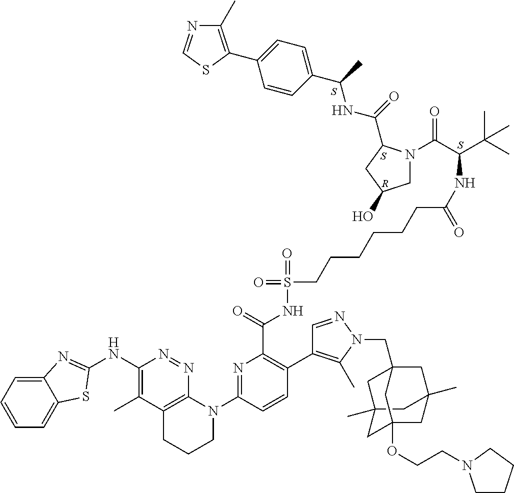 |
| | 6-[3-(1,3-benzothiazol-2-ylamino)-4-methyl-6,7-dihydro-5H-pyrido[2,3- |
| | c]pyridazin-8-yl]-3-[1-[[3,5-dimethyl-7-(2-pyrrolidin-1-ylethoxy)-1- |
| | adamantyl]methyl]-5-methyl-pyrazol-4-yl]-N-[7-[(1S)-1-[(2S,4R)-4-hydroxy- |
| | 2-[(1S)-1-[4-(4-methylthiazol-5-yl)phenyl]ethyl]carbamoyl]pyrrolidine-1- |
| | carbonyl]-2,2-dimethyl-propyl]amino]-7-oxo-heptyl]sulfonyl-pyridine-2- |
| | carboxamide |
| 173 | 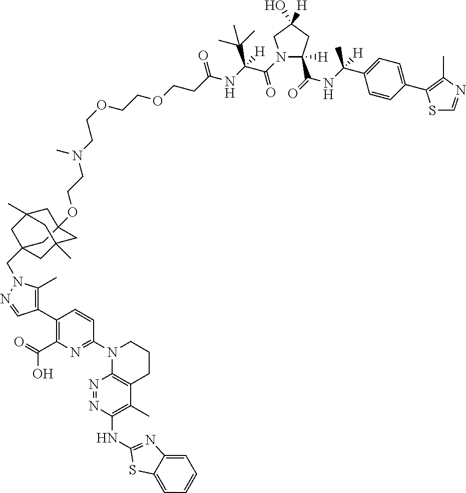 [SMILES: Cc1ncsc1-c1ccc([C@H](C)NC(=O)[C@@H]2C[C@@H](O)CN2C(=O)[C@@H](NC(=O)CCOCCOCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1] |
| | 6-[3-(1,3-benzothiazol-2-ylamino)-4-methyl-6,7-dihydro-5H-pyrido[2,3- |
| | c]pyridazin-8-yl]-3-[1-[[3-[2-[2-[2-[3-[[(1S)-1-[(2S,4R)-4-hydroxy-2-[[(1S)-1- |
| | [4-(4-methylthiazol-5-yl)phenyl]ethyl]carbamoyl]pyrrolidine-1-carbonyl]-2,2- |
| | dimethyl-propyl]amino]-3-oxo-propoxy]ethoxy]ethyl-methyl-amino]ethoxy]- |
| | 5,7-dimethyl-1-adamantyl]methyl]-5-methyl-pyrazol-4-yl]pyridine-2- |
| | carboxylic acid |
| 174 | 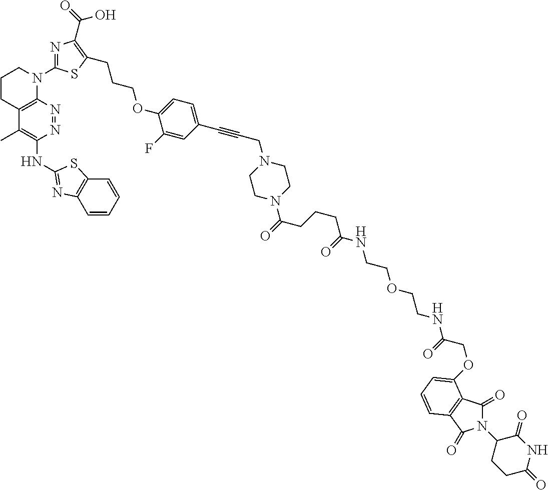 [SMILES: Cc1c(Nc2nc3ccccc3s2)nnc2c1CCCN2c1nc(C(=O)O)c(CCCOc2ccc(C#CCN3CCN(C(=O)CCCC(=O)NCCOCCNC(=O)COc4cccc5c4C(=O)N(C4CCC(=O)NC4=O)C5=O)CC3)cc2F)s1] |
| | 2-[3-(1,3-benzothiazol-2-ylamino)-4-methyl-6,7-dihydro-5H-pyrido[2,3- |
| | c]pyridazin-8-yl]-5-[3-[4-[3-[4-[5-[2-[2-[[2-[2-(2,6-dioxo-3-piperidyl)-1,3- |
| | dioxo-isoindolin-4-yl]oxyacetyl]amino]ethoxy]ethylamino]-5-oxo- |
| | pentanoyl]piperazin-1-yl]prop-1-ynyl]-2-fluoro-phenoxy]propyl]thiazole-4- |
| | carboxylic acid |

### Pharmaceutical Compositions and Methods of Treatment

Further provided herein are therapeutic uses of the disclosed compounds and compositions. An exemplary embodiment is an compound, composition, or pharmaceutical composition (e.g., any of the exemplary compounds, compositions, or pharmaceutical compositions disclosed herein) for use in treating a subject having or suspected of having a cancer (e.g., a Bcl-xL-mediated cancer). Another exemplary embodiment is a use of a compound, composition, or pharmaceutical composition (e.g., any of the exemplary compounds, compositions, or pharmaceutical compositions disclosed herein) in treating a subject having or suspected of having a cancer (e.g., a Bcl-xL-mediated cancer). Another exemplary embodiment is a use of a compound, composition, or pharmaceutical composition (e.g., any of the exemplary compounds, compositions, or pharmaceutical compositions disclosed herein) in a method of manufacturing a medicament for treating a subject having or suspected of having a cancer (e.g., a Bcl-xL-mediated cancer).

The therapeutic compositions used in the practice of the foregoing methods may be formulated into pharmaceutical compositions comprising a pharmaceutically acceptable carrier suitable for the desired delivery method. An exemplary embodiment is a pharmaceutical composition comprising compound of the present disclosure and a pharmaceutically acceptable carrier, e.g., one suitable for a chosen means of administration, e.g., intravenous administration. The pharmaceutical composition may also comprise one or more additional inactive and/or therapeutic agents that are suitable for treating or preventing, for example, a cancer (e.g., a standard-of-care agent, etc.). The pharmaceutical composition may also comprise one or more carrier, excipient, and/or stabilizer components, and the like. Methods of formulating such pharmaceutical compositions and suitable formulations are known in the art (see, e.g., “Remington's Pharmaceutical Sciences,” Mack Publishing Co., Easton, PA).

Suitable carriers include any material that, when combined with the therapeutic composition, retains the anti-tumor function of the therapeutic composition and is generally non-reactive with the patient's immune system. Pharmaceutically acceptable carriers include any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol, mesylate salt, and the like, as well as combinations thereof. In many cases, isotonic agents are included, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the compounds of this disclosure.

A pharmaceutical composition of the present disclosure can be administered by a variety of methods known in the art. The route and/or mode of administration may vary depending upon the desired results. In some embodiments, the therapeutic formulation is solubilized and administered via any route capable of delivering the therapeutic composition to the cancer site. Potentially effective routes of administration include, but are not limited to, parenteral (e.g., intravenous, subcutaneous), intraperitoneal, intramuscular, intratumor, intradermal, intraorgan, orthotopic, and the like. In some embodiments, the administration is intravenous, subcutaneous, intraperitoneal, or intramuscular. The pharmaceutically acceptable carrier should be suitable for the route of administration, e.g., intravenous or subcutaneous administration (e.g., by injection or infusion). Depending on the route of administration, the active compound(s), i.e., the compound and/or any additional therapeutic agent, may be coated in a material to protect the compound(s) from the action of acids and other natural conditions that may inactivate the compound(s). Administration can be either systemic or local.

The therapeutic compositions disclosed herein may be sterile and stable under the conditions of manufacture and storage, and may be in a variety of forms. These include, for example, liquid, semi-solid, and solid dosage forms, such as liquid solutions (e.g., injectable and infusible solutions), dispersions or suspensions, tablets, pills, powders, liposomes, and suppositories. The form depends on the intended mode of administration and therapeutic application. In some embodiments, the disclosed compounds can be incorporated into a pharmaceutical composition suitable for parenteral administration. The injectable solution may be composed of either a liquid or lyophilized dosage form in a flint or amber vial, ampule, or pre-filled syringe, or other known delivery or storage device. In some embodiments, one or more of the compounds or pharmaceutical compositions is supplied as a dry sterilized lyophilized powder or water free concentrate in a hermetically sealed container and can be reconstituted (e.g., with water or saline) to the appropriate concentration for administration to a subject.

Typically, a therapeutically effective amount or efficacious amount of a disclosed composition, e.g., a disclosed compound, is employed in the pharmaceutical compositions of the present disclosure. The composition, e.g., one comprising a compound disclosed herein, may be formulated into a pharmaceutically acceptable dosage form by conventional methods known in the art. Dosages and administration protocols for the treatment of cancers using the foregoing methods will vary with the method and the target cancer, and will generally depend on a number of other factors appreciated in the art.

Dosage regimens for compositions disclosed herein, e.g., those comprising compounds alone or in combination with at least one additional inactive and/or active therapeutic agent, may be adjusted to provide the optimum desired response (e.g., a therapeutic response). For example, a single bolus of one or both agents may be administered at one time, several divided doses may be administered over a predetermined period of time, or the dose of one or both agents may be proportionally increased or decreased as indicated by the exigencies of the therapeutic situation. In some embodiments, treatment involves single bolus or repeated administration of the compound preparation via an acceptable route of administration. In some embodiments, the compound is administered to the patient daily, weekly, monthly, or any time period in between. For any particular subject, specific dosage regimens may be adjusted over time according to the individual's need, and the professional judgment of the treating clinician. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Dosage values for compositions comprising compounds disclosed herein and/or any additional therapeutic agent(s), may be selected based on the unique characteristics of the active compound(s), and the particular therapeutic effect to be achieved. A physician or veterinarian can start doses of the compound employed in the pharmaceutical composition at levels lower than that required to achieve the desired therapeutic effect and gradually increase the dosage until the desired effect is achieved. In general, effective doses of the compositions of the present disclosure, for the treatment of a cancer may vary depending upon many different factors, including means of administration, target site, physiological state of the patient, whether the patient is human or an animal, other medications administered, and whether treatment is prophylactic or therapeutic. The selected dosage level may also depend upon a variety of pharmacokinetic factors including the activity of the particular compositions of the present disclosure employed, or the ester, salt, or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors. Treatment dosages may be titrated to optimize safety and efficacy.

Toxicity and therapeutic efficacy of compounds provided herein can be determined by standard pharmaceutical procedures in cell culture or in animal models. For example, LD50, ED50, EC50, and IC50 may be determined, and the dose ratio between toxic and therapeutic effects (LD50/ED50) may be calculated as the therapeutic index. The data obtained from in vitro and in vivo assays can be used in estimating or formulating a range of dosage for use in humans. For example, the compositions and methods disclosed herein may initially be evaluated in xenogeneic cancer models (e.g., an NCI-H929 multiple myeloma mouse model).

In some embodiments, a compound disclosed herein or a composition comprising a compound is administered on a single occasion. In other embodiments, a compound or composition comprising the compound is administered on multiple occasions. Intervals between single dosages can be, e.g., daily, weekly, monthly, or yearly. Intervals can also be irregular, based on measuring blood levels of the administered agent (e.g., the compound) in the patient in order to maintain a relatively consistent plasma concentration of the agent. The dosage and frequency of administration of a compound or composition comprising the compound may also vary depending on whether the treatment is prophylactic or therapeutic. In prophylactic applications, a relatively low dosage may be administered at relatively infrequent intervals over a long period of time. Some patients continue to receive treatment for the rest of their lives. In therapeutic applications, a relatively higher dosage at relatively shorter intervals is sometimes required until progression of the disease is reduced or terminated, and preferably until the patient shows partial or complete amelioration of one or more symptoms of disease. Thereafter, the patient may be administered a lower, e.g., prophylactic regime.

In some embodiments, compounds of the present disclosure may be administered in an amount effective to sensitize tumor cells to one or more additional therapeutic agents and/or radiation therapy.

In some embodiments, compounds of the present disclosure may be administered as monotherapy, while in other embodiments the compounds may be administered adjunctive to another therapeutic agent or radiation therapy. For example, in some embodiments, methods of the present disclosure involve the further administration (in addition of at least one PROTAC compound disclosed herein) to a subject in need thereof at least one additional therapeutic agent-such as, for example, a Bcl-2 inhibitor, a taxane, a MEK inhibitor, an ERK inhibitor, or a RAF inhibitor.

The above therapeutic approaches can be combined with any one of a wide variety of additional surgical, chemotherapy, or radiation therapy regimens. In some embodiments, the compounds or compositions disclosed herein are co-formulated and/or co-administered with one or more additional therapeutic agents, e.g., one or more chemotherapeutic agents, one or more standard-of-care agents for the particular condition being treated.

Kits for use in the therapeutic and/or diagnostic applications described herein are also provided. Such kits may comprise a carrier, package, or container that is compartmentalized to receive one or more containers such as vials, tubes, and the like, each of the container(s) comprising one of the separate elements to be used in a method disclosed herein. A label may be present on or with the container(s) to indicate that a compound or composition within the kit is used for a specific therapy or non-therapeutic application, such as a prognostic, prophylactic, diagnostic, or laboratory application. A label may also indicate directions for either in vivo or in vitro use, such as those described herein. Directions and or other information may also be included on an insert(s) or label(s), which is included with or on the kit. The label may be on or associated with the container. A label may be on a container when letters, numbers, or other characters forming the label are molded or etched into the container itself. A label may be associated with a container when it is present within a receptacle or carrier that also holds the container, e.g., as a package insert. The label may indicate that the compound or composition within the kit is used for diagnosing or treating a condition, such as a cancer a described herein.

In some embodiments, a kit comprises a compound or composition comprising the compound. In some embodiments, the kit further comprises one or more additional components, including but not limited to: instructions for use; other reagents, e.g., a therapeutic agent (e.g., a standard-of-care agent); devices, containers, or other materials for preparing the compound for administration; pharmaceutically acceptable carriers; and devices, containers, or other materials for administering the compound to a subject. Instructions for use can include guidance for therapeutic applications including suggested dosages and/or modes of administration, e.g., in a patient having or suspected of having a cancer. In some embodiments, the kit comprises a compound and instructions for use of the compound in treating, preventing, and/or diagnosing a cancer.

It is known that elevated Bcl-xL expression correlates with resistance to radiation therapy and chemotherapy. Compounds of the present disclosure that may not be sufficiently effective as monotherapy to treat cancer can be administered in combination with other therapeutic agents (including non-targeted and targeted therapeutic agents) or radiation therapy (including radioligand therapy) to provide therapeutic benefit. Without wishing to be bound by theory, it is believed that the linked-drug conjugates described herein may sensitize tumor cells to the treatment with other therapeutic agents (including standard of care chemotherapeutic agents to which the tumor cells may have developed resistance) and/or radiation therapy. In some embodiments, compounds described herein, are administered to a subject having cancer in an amount effective to sensitize the tumor cells. As used herein, the term “sensitize” means that the treatment with the compound increases the potency or efficacy of the treatment with other therapeutic agents and/or radiation therapy against tumor cells.

Disclosed herein are methods of using the compositions described herein in treating a subject for a disorder, e.g., a cancer. Compound and compositions of the present disclosure may be administered alone or in combination with at least one additional inactive and/or active agent, e.g., at least one additional therapeutic agent, and may be administered in any pharmaceutically acceptable formulation, dosage, and dosing regimen. Treatment efficacy may be evaluated for toxicity as well as indicators of efficacy and adjusted accordingly. Efficacy measures include, but are not limited to, a cytostatic and/or cytotoxic effect observed in vitro or in vivo, reduced tumor volume, tumor growth inhibition, and/or prolonged survival.

In certain aspects, the present disclosure features a method of killing, inhibiting or modulating the growth of a cancer cell or tissue by disrupting the expression and/or activity of Bcl-xL and/or one or more upstream modulators or downstream targets thereof. The method may be used with any subject where disruption of Bcl-xL expression and/or activity provides a therapeutic benefit. Subjects that may benefit from disrupting Bcl-xL expression and/or activity include, but are not limited to, those having or at risk of having a cancer such as a tumor or a hematological cancer. In some embodiments, the cancer is a breast cancer, multiple myeloma, plasma cell myeloma, leukemia, lymphoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, chronic lymphocytic leukemia, prostate cancer, small cell lung cancer, or spleen cancer. In some embodiments, the cancer is a lymphoma or gastric cancer.

Exemplary methods include the steps of contacting a cell with a compound or composition described herein, in an effective amount, i.e., an amount sufficient to kill the cell. The method can be used on cells in culture, e.g., in vitro, in vivo, ex vivo, or in situ. For example, cells (e.g., cells collected by biopsy of a tumor or metastatic lesion; cells from an established cancer cell line; or recombinant cells), can be cultured in vitro in culture medium and the contacting step can be affected by adding the compound or composition to the culture medium. Alternatively, the compound or composition can be administered to a subject by any suitable administration route (e.g., intravenous, subcutaneous, or direct contact with a tumor tissue) to have an effect in vivo.

The in vivo effect of a therapeutic composition disclosed herein can be evaluated in a suitable animal model. For example, xenogeneic cancer models can be used, wherein cancer explants or passaged xenograft tissues are introduced into immune compromised animals, such as nude or SCID mice (Klein et al. (1997) Nature Med. 3:402-8). Efficacy may be predicted using assays that measure inhibition of tumor formation, tumor regression or metastasis, and the like.

In vivo assays that evaluate the promotion of tumor death by mechanisms such as apoptosis may also be used. In some embodiments, xenografts from tumor bearing mice treated with the therapeutic composition can be examined for the presence of apoptotic foci and compared to untreated control xenograft-bearing mice. The extent to which apoptotic foci are found in the tumors of the treated mice provides an indication of the therapeutic efficacy of the composition.

Further provided herein are methods of treating a disorder, e.g., a cancer. The compositions described herein can be administered to a non-human mammal or human subject for therapeutic purposes. The therapeutic methods include administering to a subject having or suspected of having a cancer a therapeutically effective amount of a composition comprising an Bcl-xL inhibitor.

An exemplary embodiment is a method of treating a subject having or suspected of having a cancer, comprising administering to the subject a therapeutically effective amount of a composition disclosed herein. In some embodiments, the cancer is a solid tumor or a hematological cancer. In some embodiments, the cancer is a breast cancer, multiple myeloma, plasma cell myeloma, leukemia, lymphoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, chronic lymphocytic leukemia, prostate cancer, small cell lung cancer, or spleen cancer. In some embodiments, the cancer is a lymphoma or gastric cancer.

An exemplary embodiment is a method of reducing or inhibiting the growth of a tumor in a subject, comprising administering to the subject a therapeutically effective amount of a PROTAC compound, composition, or pharmaceutical composition (e.g., any of the exemplary compounds, compositions, or pharmaceutical compositions disclosed herein). In some embodiments, administration of the compound, composition, or pharmaceutical composition reduces or inhibits the growth of the tumor by at least about 10%, at least about 20%, at least about 30%, at least about 40%, at least about 50%, at least about 60%, at least about 70%, at least about 80%, at least about 90%, at least about 95%, or at least about 99%, as compared to growth in the absence of treatment.

In certain aspects, the present disclosure further provides methods of reducing or slowing the expansion of a cancer cell population comprising administering a therapeutically effective amount of a PROTAC compound or composition comprising a PROTAC compound.

Moreover, the compounds or compositions of the present disclosure may be administered to a non-human mammal for veterinary purposes or as an animal model of human disease. Regarding the latter, such animal models may be useful for evaluating the therapeutic efficacy of the disclosed compounds (e.g., testing of dosages and time courses of administration).

### Combination Therapies

In some embodiments, the present disclosure provides methods of treatment wherein the DSM-drug conjugates disclosed herein are administered in combination with one or more (e.g., 1 or 2) additional therapeutic agents. Exemplary combination partners are disclosed herein.

In certain embodiments, a combination described herein comprises a PD-1 inhibitor. In some embodiments, the PD-1 inhibitor is chosen from PDR001 (Novartis), Nivolumab (Bristol-Myers Squibb), Pembrolizumab (Merck & Co), Pidilizumab (CureTech), MED10680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is PDR001. PDR001 is also known as Spartalizumab.

In certain embodiments, a combination described herein comprises a LAG-3 inhibitor. In some embodiments, the LAG-3 inhibitor is chosen from LAG525 (Novartis), BMS-986016 (Bristol-Myers Squibb), or TSR-033 (Tesaro).

In certain embodiments, a combination described herein comprises a TIM-3 inhibitor. In some embodiments, the TIM-3 inhibitor is MBG453 (Novartis), TSR-022 (Tesaro), LY-3321367 (Eli Lily), Sym23 (Symphogen), BGB-A425 (Beigene), INCAGN-2390 (Agenus), BMS-986258 (BMS), RO-7121661 (Roche), or LY-3415244 (Eli Lilly).

In certain embodiments, a combination described herein comprises a PDL1 inhibitor. In one embodiment, the PDL1 inhibitor is chosen from FAZ053 (Novartis), atezolizumab (Genentech), durvalumab (Astra Zeneca), or avelumab (Pfizer).

In certain embodiments, a combination described herein comprises a GITR agonist. In some embodiments, the GITR agonist is chosen from GWN323 (NVS), BMS-986156, MK-4166 or MK-1248 (Merck), TRX518 (Leap Therapeutics), INCAGN1876 (Incyte/Agenus), AMG 228 (Amgen) or INBRX-110 (Inhibrx).

In some embodiments, a combination described herein comprises an IAP inhibitor. In some embodiments, the IAP inhibitor comprises LCL161 or a compound disclosed in International Application Publication No. WO 2008/016893.

In an embodiment, the combination comprises an mTOR inhibitor, e.g., RAD001 (also known as everolimus).

In an embodiment, the combination comprises a HDAC inhibitor, e.g., LBH589. LBH589 is also known as panobinostat.

In an embodiment, the combination comprises an IL-17 inhibitor, e.g., CJM112.

In certain embodiments, a combination described herein comprises an estrogen receptor (ER) antagonist. In some embodiments, the estrogen receptor antagonist is used in combination with a PD-1 inhibitor, a CDK4/6 inhibitor, or both. In some embodiments, the combination is used to treat an ER positive (ER₊) cancer or a breast cancer (e.g., an ER₊ breast cancer).

In some embodiments, the estrogen receptor antagonist is a selective estrogen receptor degrader (SERD). SERDs are estrogen receptor antagonists which bind to the receptor and result in e.g., degradation or down-regulation of the receptor (Boer K. et al., (2017) Therapeutic Advances in Medical Oncology 9(7): 465-479). ER is a hormone-activated transcription factor important for e.g., the growth, development and physiology of the human reproductive system. ER is activated by, e.g., the hormone estrogen (17beta estradiol). ER expression and signaling is implicated in cancers (e.g., breast cancer), e.g., ER positive (ER₊) breast cancer. In some embodiments, the SERD is chosen from LSZ102, fulvestrant, brilanestrant, or elacestrant.

In some embodiments, the SERD comprises a compound disclosed in International Application Publication No. WO 2014/130310, which is hereby incorporated by reference in its entirety.

In some embodiments, the SERD comprises LSZ102. LSZ102 has the chemical name: (E)-3-(4-((2-(2-(1,1-difluoroethyl)-4-fluorophenyl)-6-hydroxybenzo[b]thiophen-3-yl)oxy)phenyl)acrylic acid. In some embodiments, the SERD comprises fulvestrant (CAS Registry Number: 129453-61-8), or a compound disclosed in International Application Publication No. WO 2001/051056, which is hereby incorporated by reference in its entirety. In some embodiments, the SERD comprises elacestrant (CAS Registry Number: 722533-56-4), or a compound disclosed in U.S. Pat. No. 7,612,114, which is incorporated by reference in its entirety. Elacestrant is also known as RAD1901, ER-306323 or (6R)-6-{2-[Ethyl({4-[2-(ethylamino)ethyl]phenyl}methyl)amino]-4-methoxyphenyl}-5,6,7,8-tetrahydronaphthalen-2-ol. Elacestrant is an orally bioavailable, non-steroidal combined selective estrogens receptor modulator (SERM) and a SERD. Elacestrant is also disclosed, e.g., in Garner F et al., (2015) Anticancer Drugs 26(9):948-56. In some embodiments, the SERD is brilanestrant (CAS Registry Number: 1365888-06-7), or a compound disclosed in International Application Publication No. WO 2015/136017, which is incorporated by reference in its entirety.

In some embodiments, the SERD is chosen from RU 58668, GW7604, AZD9496, bazedoxifene, pipendoxifene, arzoxifene, OP-1074, or acolbifene, e.g., as disclosed in McDonell et al. (2015) Journal of Medicinal Chemistry 58(12) 4883-4887.

Other exemplary estrogen receptor antagonists are disclosed, e.g., in WO 2011/156518, WO 2011/159769, WO 2012/037410, WO 2012/037411, and US 2012/0071535, all of which are hereby incorporated by reference in their entirety.

In certain embodiments, a combination described herein comprises an inhibitor of Cyclin-Dependent Kinases 4 or 6 (CDK4/6). In some embodiments, the CDK4/6 inhibitor is used in combination with a PD-1 inhibitor, an estrogen receptor (ER) antagonist, or both. In some embodiments, the combination is used to treat an ER positive (ER₊) cancer or a breast cancer (e.g., an ER₊ breast cancer). In some embodiments, the CDK4/6 inhibitor is chosen from ribociclib, abemaciclib (Eli Lilly), or palbociclib.

In some embodiments, the CDK4/6 inhibitor comprises ribociclib (CAS Registry Number: 1211441-98-3), or a compound disclosed in U.S. Pat. Nos. 8,415,355 and 8,685,980, which are incorporated by reference in their entirety.

In some embodiments, the CDK4/6 inhibitor comprises a compound disclosed in International Application Publication No. WO 2010/020675 and U.S. Pat. Nos. 8,415,355 and 8,685,980, which are incorporated by reference in their entirety.

In some embodiments, the CDK4/6 inhibitor comprises ribociclib (CAS Registry Number: 1211441-98-3). Ribociclib is also known as LEE011, KISQALI®, or 7-cyclopentyl-N,N-dimethyl-2-((5-(piperazin-1-yl)pyridin-2-yl)amino)-7H-pyrrolo[2,3-d]pyrimidine-6-carboxamide.

In some embodiments, the CDK4/6 inhibitor comprises abemaciclib (CAS Registry Number: 1231929-97-7). Abemaciclib is also known as LY835219 or N-[5-[(4-Ethyl-1-piperazinyl)methyl]-2-pyridinyl]-5-fluoro-4-[4-fluoro-2-methyl-1-(1-methylethyl)-1H-benzimidazol-6-yl]-2-pyrimidinamine. Abemaciclib is a CDK inhibitor selective for CDK4 and CDK6 and is disclosed, e.g., in Torres-Guzman R et al. (2017) Oncotarget 10.18632/oncotarget.17778.

In some embodiments, the CDK4/6 inhibitor comprises palbociclib (CAS Registry Number: 571190-30-2). Palbociclib is also known as PD-0332991, IBRANCE® or 6-Acetyl-8-cyclopentyl-5-methyl-2-{[5-(1-piperazinyl)-2-pyridinyl]amino}pyrido[2,3-d]pyrimidin-7(8H)-one. Palbociclib inhibits CDK4 with an IC50 of 11 nM, and inhibits CDK6 with an IC50 of 16 nM, and is disclosed, e.g., in Finn et al. (2009) Breast Cancer Research 11(5):R77.

In certain embodiments, a combination described herein comprises an inhibitor of chemokine (C-X-C motif) receptor 2 (CXCR2). In some embodiments, the CXCR2 inhibitor is chosen from 6-chloro-3-((3,4-dioxo-2-(pentan-3-ylamino)cyclobut-1-en-1-yl)amino)-2-hydroxy-N-methoxy-N-methylbenzenesulfonamide, danirixin, reparixin, or navarixin.

In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK) (e.g., pexidartinib), or an antibody targeting CSF-1R (e.g., emactuzumab or FPA008). In some embodiments, the CSF-1/1R inhibitor is BLZ945. In some embodiments, the CSF-1/1R binding agent is MCS110. In other embodiments, the CSF-1/1R binding agent is pexidartinib.

In certain embodiments, a combination described herein comprises a c-MET inhibitor. c-MET, a receptor tyrosine kinase overexpressed or mutated in many tumor cell types, plays key roles in tumor cell proliferation, survival, invasion, metastasis, and tumor angiogenesis. Inhibition of c-MET may induce cell death in tumor cells overexpressing c-MET protein or expressing constitutively activated c-MET protein. In some embodiments, the c-MET inhibitor is chosen from capmatinib (INC280), JNJ-3887605, AMG 337, LY2801653, MSC2156119J, crizotinib, tivantinib, or golvatinib.

In certain embodiments, a combination described herein comprises a transforming growth factor beta (also known as TGF-β TGFβ, TGFb, or TGF-beta, used interchangeably herein) inhibitor. In some embodiments, the TGF-β inhibitor is chosen from fresolimumab or XOMA 089.

In certain embodiments, a combination described herein comprises an adenosine A2a receptor (A2aR) antagonist (e.g., an inhibitor of A2aR pathway, e.g., an adenosine inhibitor, e.g., an inhibitor of A2aR or CD-73). In some embodiments, the A2aR antagonist is used in combination with a PD-1 inhibitor, and one or more (e.g., two, three, four, five, or all) of a CXCR2 inhibitor, a CSF-1/1R binding agent, LAG-3 inhibitor, a GITR agonist, a c-MET inhibitor, or an IDO inhibitor. In some embodiments, the combination is used to treat a pancreatic cancer, a colorectal cancer, a gastric cancer, or a melanoma (e.g., a refractory melanoma). In some embodiments, the A2aR antagonist is chosen from PBF509 (NIR178) (Palobiofarma/Novartis), CP1444/V81444 (Corvus/Genentech), AZD4635/HTL-1071 (AstraZeneca/Heptares), Vipadenant (Redox/Juno), GBV-2034 (Globavir), AB928 (Arcus Biosciences), Theophylline, Istradefylline (Kyowa Hakko Kogyo), Tozadenant/SYN-115 (Acorda), KW-6356 (Kyowa Hakko Kogyo), ST-4206 (Leadiant Biosciences), or Preladenant/SCH 420814 (Merck/Schering). Without wishing to be bound by theory, it is believed that in some embodiments, inhibition of A2aR leads to upregulation of IL-1 b.

In certain embodiments, a combination described herein comprises an inhibitor of indoleamine 2,3-dioxygenase (IDO) and/or tryptophan 2,3-dioxygenase (TDO). In some embodiments, the IDO inhibitor is used in combination with a PD-1 inhibitor, and one or more (e.g., two, three, four, or all) of a TGF-β inhibitor, an A2aR antagonist, a CSF-1/1R binding agent, a c-MET inhibitor, or a GITR agonist. In some embodiments, the combination is used to treat a pancreatic cancer, a colorectal cancer, a gastric cancer, or a melanoma (e.g., a refractory melanoma). In some embodiments, the IDO inhibitor is chosen from (4E)-4-[(3-chloro-4-fluoroanilino)-nitrosomethylidene]-1,2,5-oxadiazol-3-amine (also known as epacadostat or INCB24360), indoximod (NLG8189), (1-methyl-D-tryptophan), α-cyclohexyl-5H-Imidazo[5,1-a]isoindole-5-ethanol (also known as NLG919), indoximod, BMS-986205 (formerly F001287).

In certain embodiments, a combination described herein comprises a Galectin, e.g., Galectin-1 or Galectin-3, inhibitor. In some embodiments, the combination comprises a Galectin-1 inhibitor and a Galectin-3 inhibitor. In some embodiments, the combination comprises a bispecific inhibitor (e.g., a bispecific antibody molecule) targeting both Galectin-1 and Galectin-3. In some embodiments, the Galectin inhibitor is used in combination with one or more therapeutic agents described herein. In some embodiments, the Galectin inhibitor is chosen from an anti-Galectin antibody molecule, GR-MD-02 (Galectin Therapeutics), Galectin-3C (Mandal Med), Anginex, or OTX-008 (OncoEthix, Merck).

In some embodiments, a combination described herein comprises an inhibitor of the MAP kinase pathway including ERK inhibitors, MEK inhibitors and RAF inhibitors.

In some embodiments, a combination described herein comprises a MEK inhibitor. In some embodiments, the MEK inhibitor is chosen from Trametinib, selumetinib, AS703026, BIX 02189, BIX 02188, CI-1040, PD0325901, PD98059, U0126, XL-518, G-38963, or G02443714.

In some embodiments, the MEK inhibitor is trametinib. Trametinib is also known as JTP-74057, TMT212, N-(3-{3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl}phenyl)acetamide, or Mekinist (CAS Number 871700-17-3).

In some embodiments, the MEK inhibitor comprises selumetinib which has the chemical name: (5-[(4-bromo-2-chlorophenyl)amino]-4-fluoro-N-(2-hydroxyethoxy)-1-methyl-1H-benzimidazole-6-carboxamide. Selumetinib is also known as AZD6244 or ARRY 142886, e.g., as described in PCT Publication No. WO2003077914.

In some embodiments, the MEK inhibitor comprises AS703026, BIX 02189 or BIX 02188.

In some embodiments, the MEK inhibitor comprises 2-[(2-Chloro-4-iodophenyl)amino]-N-(cyclopropylmethoxy)-3,4-difluoro-benzamide (also known as CI-1040 or PD184352), e.g., as described in PCT Publication No. WO2000035436.

In some embodiments, the MEK inhibitor comprises N-[(2R)-2,3-Dihydroxypropoxy]-3,4-difluoro-2-[(2-fluoro-4-iodophenyl)amino]-benzamide (also known as PD0325901), e.g., as described in PCT Publication No. WO2002006213.

In some embodiments, the MEK inhibitor comprises 2′-amino-3′-methoxyflavone (also known as PD98059) which is available from Biaffin GmbH & Co., KG, Germany.

In some embodiments, the MEK inhibitor comprises 2,3-bis[amino[(2-aminophenyl)thio]methylene]-butanedinitrile (also known as U0126), e.g., as described in U.S. Pat. No. 2,779,780.

In some embodiments, the MEK inhibitor comprises XL-518 (also known as GDC-0973) which has a CAS No. 1029872-29-4 and is available from ACC Corp.

In some embodiments, the MEK inhibitor comprises G-38963. In some embodiments, the MEK inhibitor comprises G02443714 (also known as AS703206).

Additional examples of MEK inhibitors are disclosed in WO 2013/019906, WO 03/077914, WO 2005/121142, WO 2007/04415, WO 2008/024725 and WO 2009/085983, the contents of which are incorporated herein by reference. Further examples of MEK inhibitors include, but are not limited to, 2,3-Bis[amino[(2-aminophenyl)thio]methylene]-butanedinitrile (also known as U0126 and described in U.S. Pat. No. 2,779,780); (3S,4R,5Z,8S,9S,11E)-14-(Ethylamino)-8,9,16-trihydroxy-3,4-dimethyl-3,4,9,19-tetrahydro-1H-2-benzoxacyclotetradecine-1,7(8H)-dione] (also known as E6201, described in PCT Publication No. WO2003076424); vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); pimasertib (AS-703026, CAS 1204531-26-9); 2-(2-Fluoro-4-iodophenylamino)-N-(2-hydroxyethoxy)-1,5-dimethyl-6-oxo-1,6-dihydropyridine-3-carboxamide (AZD 8330); and 3,4-Difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655).

In some embodiments, a combination described herein comprises a RAF inhibitor.

RAF inhibitors include, but are not limited to, Vemurafenib (or Zelboraf®, PLX-4032, CAS 918504-65-1), GDC-0879, PLX-4720 (available from Symansis), Dabrafenib (or GSK2118436), LGX 818, CEP-32496, UI-152, RAF 265, Regorafenib (BAY 73-4506), CCT239065, or Sorafenib (or Sorafenib Tosylate, or Nexavar®).

In some embodiments, the RAF inhibitor is Dabrafenib.

In some embodiments, the RAF inhibitor is LXH254.

In some embodiments, a combination described herein comprises an ERK inhibitor.

ERK inhibitors include, but are not limited to, LTT462, ulixertinib (BVD-523), LY3214996, GDC-0994, KO-947 and MK-8353.

In some embodiments, the ERK inhibitor is LTT462. LTT462 is 4-(3-amino-6-((1S,3S,4S)-3-fluoro-4-hydroxy¬cyclohexyl)pyrazin-2-yl)-N—((S)-1-(3-bromo-5-fluorophenyl)-2-(methylamino)ethyl)-2-fluorobenzamide and is the compound of the following structure:

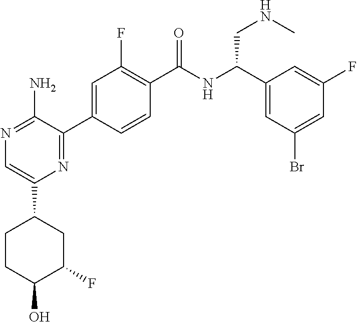 [SMILES: CNC[C@@H](NC(=O)c1ccc(-c2nc([C@H]3CC[C@H](O)[C@@H](F)C3)cnc2N)cc1F)c1cc(F)cc(Br)c1]

The preparation of LTT462 is described in PCT patent application publication WO2015/066188. LTT462 is an inhibitor of extracellular signal-regulated kinases 1 and 2 (ERK 1/2).

In some embodiments, a combination described herein comprises a taxane, a MEK inhibitor, an ERK inhibitor, or a RAF inhibitor.

In some embodiments, a combination described herein comprises at least two inhibitors selected, independently, from a MEK inhibitor, an ERK inhibitor, and a RAF inhibitor.

In some embodiments, a combination described herein comprises an anti-mitotic drug.

In some embodiments, a combination described herein comprises a taxane.

Taxanes include, but are not limited to, docetaxel, paclitaxel, or cabazitaxel. In some embodiments, the taxane is docetaxel.

In some embodiments, a combination described herein comprises a topoisomerase inhibitor.

Topoisomerase inhibitors include, but are not limited to, topotecan, irinotecan, camptothecin, diflomotecan, lamellarin D, ellipticines, etoposide (VP-16), teniposide, doxorubicin, daunorubicin, mitoxantrone, amsacrine, aurintricarboxylic acid, and HU-331.

In one embodiment, a combination described herein includes an interleukin-1 beta (IL-1p) inhibitor. In some embodiments, the IL-1p inhibitor is chosen from canakinumab, gevokizumab, Anakinra, or Rilonacept.

In certain embodiments, a combination described herein comprises an IL-15/IL-15Ra complex. In some embodiments, the IL-15/IL-15Ra complex is chosen from NIZ985 (Novartis), ATL-803 (Altor) or CYP0150 (Cytune).

In certain embodiments, a combination described herein comprises a mouse double minute 2 homolog (MDM2) inhibitor. The human homolog of MDM2 is also known as HDM2. In some embodiments, an MDM2 inhibitor described herein is also known as a HDM2 inhibitor. In some embodiments, the MDM2 inhibitor is chosen from HDM201 or CGM097.

In an embodiment the MDM2 inhibitor comprises (S)-1-(4-chlorophenyl)-7-isopropoxy-6-methoxy-2-(4-(methyl(((1 r,4S)-4-(4-methyl-3-oxopiperazin-1-yl)cyclohexyl)methyl)amino)phenyl)-1,2-dihydroisoquinolin-3(4H)-one (also known as CGM097) or a compound disclosed in PCT Publication No. WO 2011/076786 to treat a disorder, e.g., a disorder described herein). In one embodiment, a therapeutic agent disclosed herein is used in combination with CGM097.

In some embodiments, a combination described herein comprises a hypomethylating agent (HMA). In some embodiments, the HMA is chosen from decitabine or azacitidine.

In certain embodiments, a combination described herein comprises an inhibitor acting on any pro-survival proteins of the Bcl2 family. In certain embodiments, a combination described herein comprises a Bcl-2 inhibitor. In some embodiments, the Bcl-2 inhibitor is venetoclax

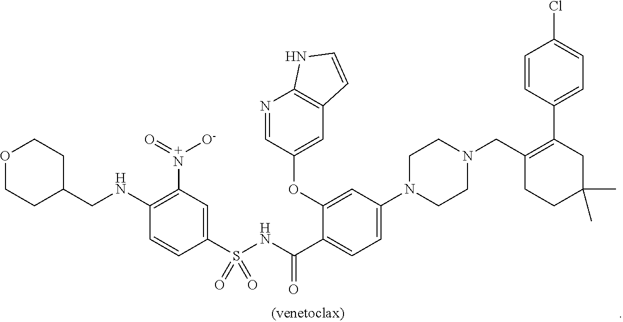 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

In one embodiment, the Bcl-2 inhibitor is selected from the compounds described in WO 2013/110890 and WO 2015/011400. In some embodiments, the Bcl-2 inhibitor comprises navitoclax (ABT-263), ABT-737, BP1002, SPC2996, APG-1252, obatoclax mesylate (GX15-070MS), PNT2258, Zn-d5, BGB-11417, or oblimersen (G3139). In some embodiments, the Bcl-2 inhibitor is N-(4-hydroxyphenyl)-3-[6-[(3S)-3-(morpholinomethyl)-3,4-dihydro-1H-isoquinoline-2-carbonyl]-1,3-benzodioxol-5-yl]-N-phenyl-5,6,7,8-tetrahydroindolizine-1-carboxamide, compound A1:

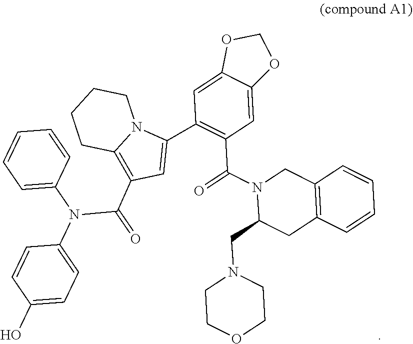 [SMILES: O=C(c1cc(-c2cc3c(cc2C(=O)N2Cc4ccccc4C[C@H]2CN2CCOCC2)OCO3)n2c1CCCC2)N(c1ccccc1)c1ccc(O)cc1]

In some embodiments, the Bcl-2 inhibitor is (S)-5-(5-chloro-2-(3-(morpholinomethyl)-1,2,3,4-tetrahydroisoquinoline-2-carbonyl)phenyl)-N-(5-cyano-1,2-dimethyl-1H-pyrrol-3-yl)-N-(4-hydroxyphenyl)-1,2-dimethyl-1H-pyrrole-3-carboxamide), compound A2:

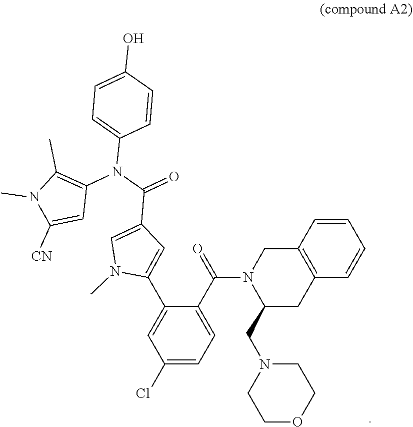 [SMILES: Cc1c(N(C(=O)c2cc(-c3cc(Cl)ccc3C(=O)N3Cc4ccccc4C[C@H]3CN3CCOCC3)n(C)c2)c2ccc(O)cc2)cc(C#N)n1C]

In one embodiment, the DSM-drug conjugates or combinations disclosed herein are suitable for the treatment of cancer in vivo. For example, the combination can be used to inhibit the growth of cancerous tumors. The combination can also be used in combination with one or more of: a standard of care treatment (e.g., for cancers or infectious disorders), a vaccine (e.g., a therapeutic cancer vaccine), a cell therapy, a hormone therapy (e.g., with anti-estrogens or anti-androgens), a radiation therapy, surgery, or any other therapeutic agent or modality, to treat a disorder herein. For example, to achieve antigen-specific enhancement of immunity, the combination can be administered together with an antigen of interest. A combination disclosed herein can be administered in either order or simultaneously.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

Exemplary compounds were synthesized using exemplary methods described in this example. All reagents obtained from commercial sources were used without further purification. Anhydrous solvents were obtained from commercial sources and used without further drying.

Column Chromatography: Flash chromatography was performed on CombiFlash® Rf (Teledyne ISCO) with pre-packed silica-gel cartridges (Macherey-Nagel Chromabond® Flash). Thin layer chromatography was conducted with 5×10 cm plates coated with Merck Type 60 F254 silica-gel.

Microwave Heating: Microwave heating was performed in CEM Discover® instrument, or with an Anton Paar Monowave Microwave Reactor.

NMR: ¹H-NMR measurements were performed on 400 MHz Bruker® Avance or 500 MHz Avance Neo spectrometer, using DMSO-d₆ or CDCl₃ as solvent. ¹H NMR data is in the form of delta values, given in part per million (ppm), using the residual peak of the solvent (2.50 ppm for DMSO-d₆ and 7.26 ppm for CDCl₃) as internal standard. Splitting patterns are designated as: s (singlet), d (doublet), t (triplet), q (quartet), quint (quintet), m (multiplet), br s (broad singlet), dd (doublet of doublets), td (triplet of doublets), dt (doublet of triplets), ddd (doublet of doublet of doublets).

IR: IR measurements were performed on a Bruker® Tensor 27 equipped with ATR Golden Gate® device (SPECAC).

Mass Spectrometry: High-Resolution MS measurements (HRMS) were performed on a LTQ OrbiTrap® Velos Pro mass spectrometer (ThermoFisher Scientific). Samples were dissolved in CH₃CN/H₂O (2/1:v/v) at a concentration range from 0.01 to 0.05 mg/mL approximately and introduced in the source by an injection of 2 μL in a flow of 0.1 mL/min. ESI ionization parameters were as follow: 3.5 kV and 350° C. transfer ion capillary. All the spectra were acquired in positive ion mode with a resolving power of 30 000 or 60 000 using a lock mass.

Some of the high-resolution mass spectra were acquired on an Agilent® 6545 quadrupole time-of-flight mass spectrometer equipped with a Dual AJS electrospray ion source in positive ion mode. Injections of 0.5 μl were directed to the mass spectrometer at a flow rate 1.5 ml/min (5 mM ammonium-formate in water and acetonitrile gradient program), using an Agilent® 1290 Infinity II HPLC system. Jet Stream parameters: drying gas (N₂) flow and temperature: 10.0 l/min and 300° C., respectively; nebulizer gas (N₂) pressure: 40 psi; capillary voltage: 2500 V; sheath gas flow and temperature: 300° C. and 10.0 l/min; QTOFMS parameters: fragmentor voltage: 100 V; skimmer potential: 65 V; OCT 1 RF Vpp:750 V. Full-scan mass spectra were acquired over the m/z range 105-1700 at an acquisition rate of 995.6 ms/spectrum and processed by Agilent MassHunter B.04.00 software.

### UPLC®-MS:

UPLC®-MS data were acquired using an instrument with the following parameters (Table 8):

TABLE 8
UPLC ®-MS Parameters

| | |
|---|---|
| Instruments: | Waters Aquity A-class with diode array UV detector “PDA” and |
| | “ZQ detector 2” mass device and MassLinks software. |
| ZQ detector 2: | MS scan from 0.15 to 6 min and from 100 to 2372 Da |
| PDA detector: | from 190 to 400 nm |
| Column: | Aquity UPLC ® BEH column C18, 1.7 μm, 130 Å, 2.1 × 50 mm |
| | Column used at 40° C. with a flowrate of 0.6 mL/min |
| Solvent A: | water + 0.02% TFA. Solvent B: acetnitrile + 0.02% TFA |
| gradient | from 2% B to 100% B in 5 min, then 0.3 min washing with 100% B |
| | and 0.5 min equilibration at 2% B for the next injection (total |
| | gradient of 6 min) |

### Preparative HPLC (Prep-HPLC):

Preparative HPLC (“Prep-HPLC”) data were acquired using an instrument with the parameters of Table 9, or using an instrument with the parameters of Table 10:

TABLE 9
Prep-HPLC Parameters (Interchim Method)

| | |
|---|---|
| Instrument(s) | Interchim Puriflash 4100 ® with a maximum of 100 bars and a |
| | maximum flowrate of 250 ml/min, or |
| | Interchim Puriflash 4250 ® with a maximum of 250 bars and a |
| | maximum flowrate of 250 ml/min |
| | Quaternary solvent pump with the possibility to use 4 solvents at |
| | the same time in a gradient |
| UV: | 2 wavelengths for the collection between 200 and 400 nm |
| Collection: | 8 ml or 32 ml tubes |
| Columns: | Waters Xbridge ® 10 μm |

TABLE 10
Prep-HPLC Parameters (Teledyne Method)

| | |
|---|---|
| Instrument(s) | Teledyne ISCO CombiFlash RF + Lumen |
| UV: | 2 wavelengths for the collection between 200 and 400 nm |
| Collection: | 8 ml or 32 ml tubes |
| Columns: | RediSep Rf Gold ® Reversed-phase C18 columns |
| | (30 g, 50 g, 100 g, 150 g or 275 g) |

Three Prep-HPLC methods were used:
1) TFA method: solvent: A=water+0.05% TFA, B=acetonitrile+0.05% TFA, gradient from 5 to 100% B in 15 to 30 CV
2) NH₄HCO₃ method: solvent: A=water+0.02 M NH₄HCO₃, B=acetonitrile/water 80/20+0.02 M NH₄HCO₃, gradient from 5 to 100% B in 15 to 30 CV
3) Neutral method: solvent: A=water, B=acetonitrile, gradient from 5 to 100% B in 15 to 30 CV

Certain compounds of the present disclosure were purified on Teledyne EZ system with a Gemini-NX® 10 μM C18, 250 mm×50 mm i.d. column running at a flow rate of 118 mL min⁻¹ with UV diode array detection (210-400 nm) using 5-25 mM aqueous NH₄HCO₃ solution and MeCN or IPA, or 0.1% TFA in water and MeCN as eluents.

All the fractions containing the pure compound were combined and directly freeze-dried to afford the compound as an amorphous powder.

Chemical naming: IUPAC-preferred names were generated using ChemAxon's ‘Structure to Name’ (s2n) functionality within MarvinSketch or JChem for Excel (JChem versions 16.6.13-18.22.3), or with the chemical naming functionality provided by Biovia@Draw 4.2.

Abbreviations: The following abbreviation are used in the examples below.
Boc₂O di-tert-butyl dicarbonate
DCM dichloromethane
DEA N,N-diethylamine
DIAD diisopropyl azodicarboxylate
DIPEA or DIEA N,N-diisopropylethylamine
DMAP 4-dimethylaminopyridine
DMF dimethylformamide
DMSO dimethylsulfoxide
DTAD di-tert-butyl azodicarboxylate
EDC*HCl N-ethyl, N′-dimethylamino-propylcarbodiimide, hydrochloride
EEDQ ethyl 2-ethoxy-2H-quinoline-1-carboxylate
EtOAc ethyl acetate
Fmoc fluorenylmethyloxycarbonyl
h hour(s)
HATU [bis(dimethylamino)methylene]-1H-1,2,3-triazolo[4,5-b]pyridinium 3-oxide hexafluorophosphate
HBTU [benzotriazol-1-yloxy(dimethylamino)methylene]-dimethyl-ammonium; hexafluorophosphate
HFxPyr Hydrogen fluoride pyridine
HOAt 1-hydroxy-7-azabenzotriazole
HOBt 1-hydroxy-benzotriazole
IPA iso-propanol
La(OiPr)₃ lanthanum(III)isopropoxide
min minute(s)
MeCN acetonitrile
MgSO₄ magnesium sulfate
MTBE methyl tert-butyl ether
Na₂SO₄ sodium sulfate
NH₄Cl ammonium chloride
NMP N-methylpyrrolidone
Pd(Ataphos)₂Cl₂ bis(di-tert-butyl(4-dimethylaminophenyl)phosphine) dichloropalladium(II)
Pd/C palladium (0) on charcoal
Pd₂(dba)₃ tris(dibenzylideneacetone)dipalladium(0)
PTFE polytetrafluoroethylene
RT room temperature
SOCl₂ thionyl chloride
TBAF Tetra-n-butylammonium fluoride
TBAI tetrabutylammonium, iodide
TEA N,N-diethylethanamine
TFA trifluoroacetic acid
THF tetrahydrofuran
THTPA tris(3-hydroxypropyltriazolyl-methyl)amine]
TSTU [dimethylamino-(2,5-dioxopyrrolidin-1-yl)oxy-methylene]-dimethyl-ammonium; tetrafluoroborate
VHL von Hippel-Lindau protein
XantPhos 4,5-Bis(diphenylphosphino)-9,9-dimethylxanthene

### A. Synthesis and Characterization of Bcl-xL Payload Compounds

Exemplary Bcl-xL payloads were synthesized using exemplary methods described in this example.

### Mitsunobu General Procedure

To the mixture of 1.0-1.5 eq. of aliphatic alcohol, 1 eq. of carbamate/phenol, and 1-2 eq. triphenylphosphine in THF or toluene (5 mL/mmol) were added 1-3 eq. of ditertbutyl azodicarboxylate/diisopropyl azodicarboxylate in one portion. The mixture was stirred at rt or 50° C., if necessary, for the carbamate and at rt for the phenol. After reaching an appropriate conversion the volatiles were removed under reduced pressure, the crude intermediate was purified via flash column chromatography.

Deprotection with HFIP General Procedure

Substrate in 1, 1,1,3,3,3-hexafluoropropan-2-ol (10 mL/mmol) was kept at 100-120° C. in a pressure bottle. After reaching an appropriate conversion the volatiles were removed under reduced pressure, the crude intermediate was purified via flash column chromatography.

### Buchwald General Procedure II

The mixture of 1 eq. of thiazole amine, 1.2-1.5 eq. of (Z)—N-(6-chloro-4-methyl-pyridazin-3-yl)-3-(2-trimethylsilylethoxymethyl)-1,3-benzothiazol-2-imine, 3 eq. of Cs₂CO₃, 0.1 eq. of Pd₂(dba)₃, 0.2 eq. of XantPhos and 3 eq. of DIPEA in 1,4-dioxane (5 mL/mmol) was kept at reflux. After reaching an appropriate conversion the volatiles were removed under reduced pressure, the crude intermediate was purified via flash column chromatography.

### Deprotection and Hydrolysis General Procedure

The mixture of 1 eq. of substrate and 100 eq. of HFxPyr in MeCN (15 mL/mmol) was stirred at 60° C. After reaching an appropriate conversion, the volatiles were removed under reduced pressure and the residue was suspended in a 1:1 mixture of 1,4-dioxane-water (30 mL/mmol), treated with 150 eq. of LiOH×H₂O, and stirred at 60° C. After reaching an appropriate conversion, the volatiles were removed under reduced pressure and the crude product was purified via flash column chromatography using DCM and MeOH (containing 1.2% NH₃) as eluents.

### Preparation 1: 3-(3,6-dichloro-5-methyl-pyridazin-4-yl)propan-1-ol

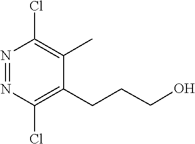

### Step A: [(pent-4-yn-1-yloxy)methyl]benzene

To an oven-dried flask was added 4-pentyn-1-ol (11.1 mL, 119 mmol, 1 eq) in THF (100 mL) and the solution was cooled to 0° C. Sodium hydride (60% dispersion; 7.13 g, 1.5 eq) was added portionwise and the mixture was allowed to stir for 30 min at 0° C. before the dropwise addition of benzyl bromide (15.6 mL, 131 mmol, 1.1 eq). The mixture was allowed to warm to ambient temperature and was stirred for 16 h, then cooled to 0° C., quenched with saturated aqueous ammonium chloride (30 mL) and diluted with water (30 mL). The mixture was extracted with ethyl acetate (2×150 mL), and the combined organic extracts were washed successively with dilute aqueous ammonium hydroxide (150 mL) and brine (100 mL), dried (magnesium sulfate) and concentrated in vacuo. Purification by automated flash column chromatography (CombiFlash Rf, 330 g RediSep™ silica cartridge) eluting with a gradient of ethyl acetate in iso-heptane afforded the desired product (19.5 g, 94%). ¹H NMR (400 MHz, Chloroform-d) δ 7.37-7.32 (m, 4H), 7.31-7.27 (m, 1H), 4.52 (s, 2H), 3.58 (t, J=6.1 Hz, 2H), 2.32 (td, J=7.1, 2.6 Hz, 2H), 1.95 (t, J=2.7 Hz, 1H), 1.83 (tt, J=7.1, 6.2 Hz, 2H); LC/MS (C₁₂H₁₄O) 175 [M+H]⁺.

### Step B: [(hex-4-yn-1-yloxy)methyl]benzene

To an oven-dried flask was added the product from Step A (19.5 g, 112 mmol, 1 eq) and tetrahydrofuran (200 mL) and the solution was cooled to −78° C. n-Butyllithium (2M solution in hexanes, 66.9 mL, 135 mmol, 1.2 eq) was added dropwise over 30 min and the reaction was stirred for 1 h then iodomethane (10.5 mL, 168 mmol, 1.5 eq) was added dropwise and the mixture was allowed to warm to 0° C. over 1 h. The reaction was quenched by the addition of saturated aqueous ammonium chloride (40 mL), diluted with water (40 mL), extracted with ethyl acetate (3×100 mL), and the combined organic extracts were successively washed with 2M aqueous sodium thiosulfate (200 mL) and brine (200 mL), dried (magnesium sulfate) and concentrated in vacuo. Purification by automated flash column chromatography (CombiFlash Rf, 330 g RediSep™ silica cartridge) eluting with a gradient of 0-10% ethyl acetate in iso-heptane afforded the desired product (19.2 g, 91%). ¹H NMR (400 MHz, DMSO-d6) δ 7.41-7.23 (m, 5H), 4.46 (s, 2H), 3.48 (t, J=6.3 Hz, 2H), 2.23-2.14 (m, 2H), 1.72 (s, 3H), 1.70-1.65 (m, 2H); LC/MS (C₁₃H₁₆O) 189 [M+H]⁺.

### Step C: 4-[3-(benzyloxy)propyl]-3,6-dichloro-5-methylpyridazine

A solution of 3,6-dichloro-1,2,4,5-tetrazine (5 g, 33.1 mmol, 1 eq) and the product from Step B (7.48 g, 39.8 mmol, 1.2 eq) in tetrahydrofuran (30 mL) was heated at 160° C. for 19 h in a sealed flask. The reaction was allowed to cool to ambient temperature then concentrated in vacuo. Purification by automated flash column chromatography (CombiFlash Rf, 220 g RediSep™ silica cartridge) eluting with a gradient of 0-30% ethyl acetate in iso-heptane afforded the desired product (7.32 g, 71%). ¹H NMR (400 MHz, DMSO-d6) δ 7.45-7.18 (m, 5H), 4.48 (s, 2H), 3.53 (t, J=5.9 Hz, 2H), 2.96-2.83 (m, 2H), 2.42 (s, 3H), 1.88-1.69 (m, 2H); LC/MS (C₁₅H₁₆C₁₂N₂O) 311 [M+H]⁺.

### Step D: 3-(3,6-dichloro-5-methyl-pyridazin-4-yl)propan-1-ol

To a cooled solution of the product from Step C (7.32 g, 23.5 mmol, 1 eq) in dichloromethane (100 mL) was added boron trichloride solution (1 M in dichloromethane; 58.8 mL, 58.8 mmol, 2.5 eq) dropwise and the mixture was allowed to stir at ambient temperature for 1 h. The reaction was quenched by the addition of methanol and concentrated in vacuo. The residue was partitioned between dichloromethane (100 mL) and saturated aqueous sodium bicarbonate (150 mL), and the organic phase was washed with brine (150 mL), dried (magnesium sulfate) and concentrated in vacuo. Purification by automated flash column chromatography (CombiFlash Rf, 80 g RediSep™ silica cartridge) eluting with a gradient of 0-80% ethyl acetate in iso-heptane afforded the desired product (4.19 g, 81%). ¹H NMR (400 MHz, DMSO-d₆) δ 4.67 (t, J=5.1 Hz, 1H), 3.49 (td, J=6.0, 5.1 Hz, 2H), 2.91-2.80 (m, 2H), 2.43 (s, 3H), 1.72-1.59 (m, 2H); LC/MS (C₈H₁₀C₁₂N₂O) 221 [M+H]⁺.

### Preparation 2: methyl 3-bromo-6-[3-(3,6-dichloro-5-methyl-pyridazin-4-yl)propylamino]pyridine-2-carboxylate

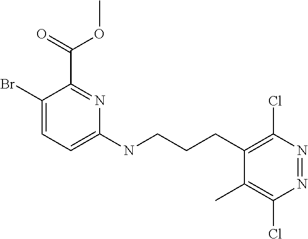

### Step A: methyl 6-[bis(tert-butoxycarbonyl)amino]-3-bromo-pyridine-2-carboxylate

To methyl 6-amino-3-bromo-pyridine-2-carboxylate (25.0 g, 108.2 mmol) and DMAP (1.3 g, 0.1 eq) in DCM (541 mL) was added Boc₂O (59.0 g, 2.5 eq) at 0° C. and the reaction mixture was stirred for 2.5 h. After the addition of a saturated solution of NaHCO₃ and extraction with DCM, the combined organic phases were dried and concentrated to afford the desired product (45.0 g, 72.3%). LC/MS (C₁₇H₂₃BrN₂O₆Na) 453 [M+Na]⁺.

### Step B: methyl 3-bromo-6-(tert-butoxycarbonylamino)pyridine-2-carboxylate

To the product from Step A (42.7 g, 74.34 mmol) in DCM (370 mL) was added TFA (17.1 mL, 3 eq) at 0° C. and the reaction mixture was stirred for 18 h. After washing with a saturated solution of NaHCO₃ and brine, the combined organic phases were dried, concentrated and purified by column chromatography (silica gel, heptane and EtOAc as eluents) to give the desired product (28.3 g, 115.2%). ¹H NMR (400 MHz, DMSO-d₆): δ ppm 10.29 (s, 1H), 8.11 (d, 1H), 7.88 (d, 1H), 3.87 (s, 3H), 1.46 (s, 9H)¹³C NMR (100 MHz, DMSO-d₆) δ ppm 165.6, 153.1, 151.8/148.3, 143.5, 116.3, 109.2, 53.2, 28.4. LC/MS (C₁₂H₁₅BrN₂O₄Na) 353 [M+Na]⁺.

### Step C: methyl 3-bromo-6-[tert-butoxycarbonyl-[3-(3,6-dichloro-5-methyl-pyridazin-4-yl)propyl]amino]pyridine-2-carboxylate

After mixing the product of Step B (748 mg, 2.2 mmol), the product of Preparation 1 (500 mg, 1 eq), and PPh₃ (593 mg, 1 eq) in toluene (10 mL), DTAD (520 mg, 1 eq) was added, and stirred at 50° C. for 30 min. Purification by column chromatography (silica gel, heptane and EtOAc as eluents) afforded the desired product (1.1 g, 91%). ¹H NMR (400 MHz, DMSO-d₆): δ ppm 8.13 (d, 1H), 7.78 (d, 1H), 3.91 (t, 2H), 3.89 (s, 3H), 2.79 (m, 2H), 2.38 (s, 3H), 1.82 (m, 2H), 1.46 (s, 9H); ¹³C NMR (100 MHz, DMSO-d₆) δ ppm 165.3, 157.6, 156.6, 153.2, 152.9, 147.2, 143.1, 142.2, 139.7, 122.6, 111.8, 82.2, 53.3, 46.4, 28.1, 27.7, 26.5, 16.3; HRMS-ESI (m/z): [M+Na]⁺ calcd for C₂₀H₂₃BrCl₂N₄NaO₄: 555.0177. found: 555.0172.

### Step D: methyl 3-bromo-6-[3-(3,6-dichloro-5-methyl-pyridazin-4-yl)propylamino]pyridine-2-carboxylate

The product from Step C (17.5 g, 32.7 mmol) in 1,1,1,3,3,3-hexafluoroisopropanol (330 mL) was stirred at 110° C. for 18 h. Purification by column chromatography (silica gel, heptane and EtOAc as eluents) afforded the desired product (9.9 g, 70%). ¹H NMR (400 MHz, DMSO-d₆): 5 ppm 7.63 (d, 1H), 7.22 (t, 1H), 6.57 (d, 1H), 3.83 (s, 3H), 3.30 (m, 2H), 2.83 (m, 2H), 2.37 (s, 3H), 1.74 (m, 2H)¹³C NMR (100 MHz, DMSO-d₆) δ ppm 166.5, 141.5, 112.6, 52.9, 40.9, 28.0, 27.0, 16.4.

### Preparation 3: tert-butyl-diphenyl-[2-[[3,5-dimethyl-7-[[5-methyl-4-(4,4,5,5-tetramethyl-1,3,2-dioxaborolan-2-yl)pyrazol-1-yl]methyl]-1-adamantyl]oxy]ethoxy]silane

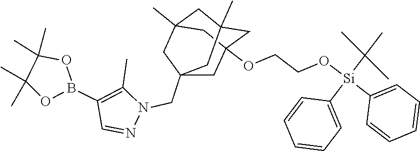 [SMILES: Cc1c(B2OC(C)(C)C(C)(C)O2)cnn1CC12CC3(C)CC(C)(C1)CC(OCCO[Si](c1ccccc1)(c1ccccc1)C(C)(C)C)(C3)C2]

### Step A: 3-bromo-5,7-dimethyladamantane-1-carboxylic acid

After stirring iron (6.7 g, 120 mmol) in bromine (30.7 mL, 600 mmol, 5 eq) at 0° C. for 1 h, 3,5-dimethyladamantane-1-carboxylic acid (25 g, 1 eq) was added and the reaction mixture was stirred at RT for 2 days. After the addition of EtOAc, the reaction mixture was treated carefully with a saturated solution of sodium-thiosulfate at 0° C. and stirred for 15 min. After filtration through a pad of Celite and rinsing with EtOAc, the organic phase was separated, washed with a saturated solution of sodium-thiosulfate and brine, dried, concentrated to give the desired product (34.28 g, 74.6%), which was used without further purification. ¹H NMR (400 MHz, DMSO-d₆): δ ppm 12.33 (br s, 1H), 2.21 (s, 2H), 1.96/1.91 (d+d, 4H), 1.50/1.43 (d+d, 4H), 1.21/1.14 (dm+dm, 2H), 0.86 (s, 6H); ¹³C NMR (100 MHz, DMSO-d₆) δ ppm 176.8, 66.8, 54.0, 48.7, 48.5, 45.7, 43.3, 35.5, 29.4; HRMS-ESI (m/z): [M−H]⁻ calcd for C₁₃H₁₈BrO₂: 285.0496. found 285.0498.

### Step B: 3-bromo-5,7-dimethyl-1-adamantyl-methanol

To the product from Step A (34.3 g, 119 mmol) in THF (77.6 mL) was added slowly a 1 M solution of BH₃-THF in THF (358 mL, 3 eq) and the reaction mixture was stirred for 18 h. After the addition of methanol and stirring for 30 min, purification by column chromatography (silica gel, heptane and MTBE as eluents) afforded the desired product (16.19 g, 49.6%). ¹H NMR (400 MHz, DMSO-d₆): δ ppm 4.51 (t, 1H), 3.05 (d, 2H), 1.91 (s, 2H), 1.91 (s, 4H), 1.19/1.09 (d+d, 2H), 1.19/1.05 (d+d, 4H), 0.85 (s, 6H)¹³C NMR (100 MHz, DMSO-d₆) δ ppm 70.4, 68.9, 54.9, 49.8, 49.3, 43.8, 41.4, 35.7, 29.7; HRMS-ESI (m/z): [M-Br]-calcd for C₁₃H₂₁O: 193.1598. found: 193.1589.

### Step C: 1-[(3-bromo-5,7-dimethyl-1-adamantyl)methyl]pyrazole

To the product from Step B (16.19 g, 59.26 mmol) and 1H-pyrazole (4.841 g, 1.2 eq) in toluene (178 mL) was added cyanomethylenetributylphosphorane (18.64 mL, 1.2 eq) in one portion and the reaction mixture was stirred at 90° C. for 2 h. Purification by column chromatography (silica gel, heptane and MTBE as eluents) afforded the desired product (17.88 g, 93%). ¹H NMR (400 MHz, DMSO-d₆): δ ppm 7.63 (d, 1H), 7.43 (d, 1H), 6.23 (t, 1H), 3.90 (s, 2H), 1.92-1.02 (m, 12H), 0.83 (s, 6H); ¹³C NMR (100 MHz, DMSO-d₆) δ ppm 139.0, 131.8, 105.2, 67.7, 61.4, 54.4/48.8/44.6, 50.4, 35.7, 29.6; HRMS-ESI (m/z): [M]⁺ calcd for C₁₆H₂₃BrN₂: 322.1045. found: 322.1014.

### Step D: 5-methyl-1-[(3-bromo-5,7-dimethyl-1-adamantyl)methyl]pyrazole

To the solution of the product from Step C (17.88 g, 55.3 mmol) in THF (277 mL) was added butyllithium (2.5 M in THF, 66 mL, 3 eq) at −78° C., then after 1 h, iodomethane (17.2 mL, 5 eq) was added. After 10 min, the reaction mixture was quenched with a saturated solution of NH₄Cl, extracted with EtOAc and the combined organic layers were dried and concentrated to give the desired product (18.7 g, 100%), which was used in the next step without further purification. ¹H NMR (400 MHz, DMSO-d₆): δ ppm 7.31 (d, 1H), 6.00 (d, 1H), 3.79 (s, 2H), 2.23 (s, 3H), 2.01 (s, 2H), 1.89/1.85 (d+d, 4H), 1.23/1.15 (d+d, 4H), 1.16/1.05 (d+d, 2H), 0.83 (s, 6H); ¹³C NMR (100 MHz, DMSO-d₆) δ ppm 139.2, 138.0, 105.2, 67.8, 57.8, 54.4, 50.6, 48.8, 44.8, 41.5, 35.7, 29.6, 11.8; HRMS-ESI (m/z): [M+H]⁺ calcd for C₁₇H₂₆BrN₂: 337.1279. found: 337.1289.

### Step E: 2-[[3,5-dimethyl-7-[(5-methylpyrazol-1-yl)methyl]-1-adamantyl]oxy]ethanol

The mixture of the product from Step D (18.7 g, 55.3 mmol), ethylene glycol (123 mL, 40 eq), and DIPEA (48.2 mL, 5 eq) was stirred at 120° C. for 6 h. After the reaction mixture was diluted with water and extracted with EtOAc, the combined organic layers were dried and concentrated to give the desired product (18.5 g, 105%), which was used in the next step without further purification. ¹H NMR (400 MHz, DMSO-d₆): δ ppm 7.29 (d, 1H), 5.99 (d, 1H), 4.45 (t, 1H), 3.78 (s, 2H), 3.39 (q, 2H), 3.32 (t, 2H), 2.23 (s, 3H), 1.34 (s, 2H), 1.27/1.21 (d+d, 4H), 1.13/1.07 (d+d, 4H), 1.04/0.97 (d+d, 2H), 0.84 (s, 6H); ¹³C NMR (100 MHz, DMSO-d₆) δ ppm 139.0, 137.8, 105.1, 74.0, 62.1, 61.5, 58.5, 50.1, 47.0, 46.1, 43.3, 39.7, 33.5, 30.2, 11.9; HRMS-ESI (m/z): [M+H]⁺ calcd for C₁₉H₃₁N₂O₂: 319.2386. found: 319.2387.

### Step F: tert-butyl-diphenyl-[2-[[3,5-dimethyl-7-[(5-methylpyrazol-1-yl)methyl]-1-adamantyl] oxy]ethoxy]silane

To the mixture of the product from Step E (17.6 g, 55.3 mmol) and imidazole (5.65 g, 1.5 eq) in DCM (150 ml) was added tert-butyl-chloro-diphenyl-silane (18.6 g, 1.2 eq) and the reaction mixture was stirred for 1 h. Purification by column chromatography (silica gel, heptane and MTBE as eluents) afforded the desired product (27.0 g, 87.8%). ¹H NMR (400 MHz, DMSO-d₆): δ ppm 7.72-7.34 (m, 1 OH), 7.29 (d, 1H), 5.99 (br s, 1H), 3.78 (s, 2H), 3.67 (t, 2H), 3.44 (t, 2H), 2.21 (s, 3H), 1.33 (s, 2H), 1.26/1.18 (d+d, 4H), 1.12/1.06 (d+d, 4H), 1.03/0.96 (d+d, 2H), 0.98 (s, 9H), 0.82 (s, 6H); ¹³C NMR (100 MHz, DMSO-d₆) δ ppm 139.0, 137.8, 105.1, 74.2, 64.4, 61.7, 58.5, 50.0, 46.9, 46.0, 43.4, 39.6, 33.5, 30.1, 27.1, 19.3, 11.9; HRMS-ESI (m/z): [M+H]⁺ calcd for C₃₅H₄₉N₂O₂Si: 557.3563. found: 557.3564.

### Step G: tert-butyl-diphenyl-[2-[[3-[(4-iodo-5-methyl-pyrazol-1-yl)methyl]-5,7-dimethyl-1-adamantyl]oxy]ethoxy]silane

To the solution of the product from Step F (27.0 g, 48.56 mmol) in DMF (243 mL) was added N-iodosuccinimide (13.6 g, 1.25 eq) and the reaction mixture was stirred for 2 h. After the dilution with water, the mixture was extracted with DCM. The combined organic layers were washed with saturated solution of sodium-thiosulphate and brine, dried, and concentrated to afford the desired product (30.1 g, 90%). ¹H NMR (400 MHz, DMSO-d₆): δ ppm 7.68-7.37 (m, 10H), 7.45 (s, 1H), 3.89 (s, 2H), 3.67 (t, 2H), 3.44 (t, 2H), 2.23 (s, 3H), 1.30 (s, 2H), 1.26/1.17 (d+d, 4H), 1.12/1.05 (d+d, 4H), 1.00/0.96 (d+d, 2H), 0.98 (s, 9H), 0.82 (s, 6H); ¹³C NMR (100 MHz, DMSO-d₆) δ ppm 142.5, 140.8, 133.7, 64.4, 61.7, 60.3, 59.9, 49.9, 46.8, 45.9, 43.2, 39.7, 33.5, 30.1, 27.1, 19.3, 12.2; HRMS-ESI (m/z): [M+H]⁺ calcd for C₃₅H₄₈IN₂O₂Si: 683.2530. found: 683.2533.

### Step H: tert-butyl-diphenyl-[2-[[3,5-dimethyl-7-[[5-methyl-4-(4,4,5,5-tetramethyl-1,3,2-dioxaborolan-2-yl)pyrazol-1-yl]methyl]-1-adamantyl]oxy]ethoxy]silane

To the product from Step G (17.5 g, 25.6 mmol) in THF (128 mL) was added chloro(isopropyl)magnesium-LiCl (1.3 M in THF, 24 mL, 1.2 eq) at 0° C., stirred for 40 min, treated with 2-isopropoxy-4,4,5,5-tetramethyl-1,3,2-dioxaborolane (15.7 mL, 3 eq), and the reaction mixture was stirred for 10 min. After dilution with a saturated solution NH₄Cl and extraction with EtOAc, the combined organic phases were concentrated and was purified by column chromatography (silica gel, heptane and MTBE as eluents) to give the desired product (15.2 g, 86.9%). ¹H NMR (400 MHz, DMSO-d₆): δ ppm 7.65 (dm, 4H), 7.47 (s, 1H), 7.45 (tm, 2H), 7.40 (tm, 4H), 3.80 (s, 2H), 3.66 (t, 2H), 3.44 (t, 2H), 2.35 (s, 3H), 1.35-0.94 (m, 12H), 1.24 (s, 12H), 0.97 (s, 9H), 0.83 (s, 6H); ¹³C NMR (100 MHz, DMSO-d₆) δ ppm 146.9, 144.3, 135.6, 130.2, 128.2, 104.7, 83.0, 74.2, 64.4, 61.7, 58.4, 30.1, 27.1, 25.2, 19.3, 12.0; HRMS-ESI (m/z): [M+H]⁺ calcd for C₄₁H₆₀BN₂O₄Si: 683.4415. found: 683.4423.

### Preparation 4: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3,5-dimethyl-7-[2-(methylamino)ethoxy]-1-adamantyl]methyl]-5-methyl-pyrazol-4-yl]pyridine-2-carboxylate

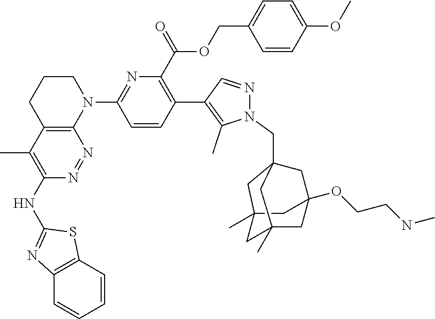

### Step A: 3-bromo-6-[3-(3,6-dichloro-5-methyl-pyridazin-4-yl)propylamino]pyridine-2-carboxylic acid

To the product of Preparation 2 (23.3 g, 53.7 mmol) in 1,4-dioxane (215 mL) and water (54 mL) was added LiOH×H₂O (13.5 g, 6 eq) and the mixture was stirred at 60° C. for 1 h. The reaction was quenched by the addition of a 1 M aqueous HCl solution and the product was filtered off to give the desired product (21.7 g, 96%). 1H NMR (500 MHz, DMSO-d₆) δ ppm 13.30 (s, 1H), 7.60 (d, 1H), 7.14 (t, 1H), 6.53 (d, 1H), 3.32 (m, 2H), 2.84 (m, 2H), 2.77 (s, 3H), 1.74 (m, 2H); 13C NMR (125 MHz, DMSO-d₆) δ ppm 167.5, 157.6, 157.4, 156.8, 149.4, 142.7, 141.4, 139.8, 112.0, 101.1, 40.9, 28.1, 27.1, 16.5; HRMS-ESI (m/z): [M+H]⁺ calcd for C₁₄H₁₄BrCl₂N₄O₂: 418.9677. found: 418.9681.

### Step B: (4-methoxyphenyl)methyl 3-bromo-6-[3-(3,6-dichloro-5-methyl-pyridazin-4-yl)propylamino]pyridine-2-carboxylate

To the product of Step A (11.2 g, 26.6 mmol), (4-methoxyphenyl)methanol (6.6 mL, 2 eq), triphenylphosphine (13.9 g, 2 eq) in toluene (266 mL) and THF (20 ml) was added dropwise DIAD (10.5 mL, 2 eq) and the reaction was kept at 50° C. for 1 h. The crude product was purified by flash chromatography on silica gel using heptane and EtOAc as eluents to give the desired product (10.4 g, 72.5%). ¹H NMR (500 MHz, DMSO-d₆) δ ppm 7.62 (d, 1H), 7.37 (dn, 2H), 7.21 (t, 1H), 6.91 (dm, 2H), 6.56 (d, 1H), 5.25 (s, 2H), 3.74 (s, 3H), 3.30 (q, 2H), 2.81 (m, 2H), 2.33 (s, 3H), 1.73 (m, 2H); ¹³C NMR (125 MHz, DMSO-d₆) δ ppm 165.9, 159.7, 157.6, 157.5, 156.8, 148.0, 142.7, 141.5, 139.7, 130.6, 127.8, 114.3, 112.6, 101.6, 67.0, 55.6, 40.9, 28.0, 27.1, 16.4; HRMS-ESI (m/z): [M+H]⁺ calcd for C₂₂H₂₂BrCl₂N₄O₃: 539.0252. found: 539.0246.

### Step C: (4-methoxyphenyl)methyl 3-[1-[[3-[2-[tert-butyl(diphenyl)silyl]oxyethoxy]-5,7-dimethyl-1-adamantyl]methyl]-5-methyl-pyrazol-4-yl]-6-[3-(3,6-dichloro-5-methyl-pyridazin-4-yl)propylamino]pyridine-2-carboxylate

The mixture of the product of Step B (27.0 g, 50.0 mmol), Preparation 3 (37.5 g, 1.1 eq), Cs₂CO₃ (48.9 g, 3 eq), Pd(AtaPhos)₂Cl₂ (2.21 g, 0.1 eq) in 1,4-dioxane (300 mL) and H₂O (50 mL) was kept at 80° C. for 6 h. After cooling and quenching by the addition of saturated aqueous NaCl solution, the mixture was extracted with EtOAc. The combined organic layers were dried and concentrated to give the desired product (54.0 g, 106%). ¹H NMR (500 MHz, DMSO-d₆) δ ppm 7.68-7.35 (m, 1 OH), 7.31 (d, 1H), 7.27 (s, 1H), 7.11 (dm, 2H), 6.98 (t, 1H), 6.83 (dm, 2H), 6.62 (d, 1H), 4.99 (s, 2H), 3.80 (s, 2H), 3.70 (s, 3H), 3.65 (t, 2H), 3.44 (t, 2H), 3.34 (q, 2H), 2.84 (m, 2H), 2.34 (s, 3H), 2.01 (s, 3H), 1.77 (m, 2H), 1.38-0.89 (m, 12H), 0.97 (s, 9H), 0.82 (s, 6H); ¹³C NMR (125 MHz, DMSO-d₆) δ ppm 140.4, 137.6, 130.1, 114.2, 110.3, 66.3, 64.4, 61.7, 59.0, 55.5, 40.9, 30.1, 28.1, 27.3, 27.1, 16.4, 10.8; HRMS-ESI (m/z): [M+H]⁺ calcd for C₅₇H₆₉C₁₂N₆O₅Si: 1015.4476. found: 1015.4474.

### Step D: (4-methoxyphenyl)methyl 3-[1-[[3-[2-[tert-butyl(diphenyl)silyl]oxyethoxy]-5,7-dimethyl-1-adamantyl]methyl]-5-methyl-pyrazol-4-yl]-6-(3-chloro-4-methyl-6,7-dihydro-5H-pyrido[2,3-c]pyridazin-8-yl)pyridine-2-carboxylate

A mixture of the product of Step C (26.0 g, 25.6 mmol) Cs₂CO₃ (9.87 g, 2 eq), DIPEA (8.9 mL, 2 eq), and Pd(Ataphos)₂Cl₂ (1.1 g, 0.1 eq) in 1,4-dioxane (128 mL) was stirred in a 200 ml pressure bottle at 110° C. for 18 h. After dilution with water, the mixture was extracted with EtOAc. The combined organic layers were dried and concentrated to give the desired product (24.8 g, 98.9%). ¹H NMR (500 MHz, DMSO-d₆) δ ppm 7.84 (d, 1H), 7.67 (d, 1H), 7.65 (d, 4H), 7.44 (t, 2H), 7.41 (s, 1H), 7.40 (t, 4H), 7.15 (d, 2H), 6.87 (d, 2H), 5.07 (s, 2H), 3.96 (t, 2H), 3.83 (s, 2H), 3.71 (s, 3H), 3.66 (t, 2H), 3.45 (t, 2H), 2.86 (t, 2H), 2.29 (s, 3H), 2.08 (s, 3H), 1.97 (qn, 2H), 1.38 (s, 2H), 1.25/1.18 (d+d, 4H), 1.18/1.12 (d+d, 4H), 1.01/0.93 (d+d, 2H), 0.97 (s, 9H), 0.82 (s, 6H); ¹³C NMR (125 MHz, DMSO-d₆) δ ppm 166.8, 159.7, 156.3, 153.6, 150.8, 147.7, 140.1, 137.6, 137.3, 136.0, 135.6, 133.8, 130.2, 130.2, 129.1, 128.2, 127.7, 123.0, 120.4, 115.6, 114.3, 74.2, 66.8, 64.4, 61.7, 59.3, 55.6, 49.9, 46.8, 46.0, 46.0, 43.3, 39.7, 33.6, 30.1, 27.1, 24.6, 21.0, 19.3, 15.5, 10.8; HRMS-ESI (m/z): [M+H]⁺ calcd for C₅₇H₆₈ClN₆O₅Si: 979.4709. found: 979.4710.

### Step E: (4-methoxyphenyl)methyl 6-(3-chloro-4-methyl-6,7-dihydro-5H-pyrido[2,3-c]pyridazin-8-yl)-3-[1-[[3-(2-hydroxyethoxy)-5,7-dimethyl-1-adamantyl]methyl]-5-methyl-pyrazol-4-yl]pyridine-2-carboxylate

To the product of Step D (3.40 g, 3.47 mmol) in THF (34.7 mL) was added a 1 M TBAF solution in THF (3.82 mL, 1.1 eq) at 0° C., and the mixture was stirred at RT for 90 min. After quenching with a saturated solution of NH₄Cl, the product was purified by flash chromatography on silica gel column using DCM and MeOH as eluents to give the desired product (1.80 g, 70%). ¹H NMR (500 MHz, DMSO-d₆) δ ppm 7.85 (d, 1H), 7.70 (d, 1H), 7.39 (s, 1H), 7.18 (d, 2H), 6.9 (d, 2H), 5.10 (s, 2H), 4.45 (t, 1H), 3.96 (t, 2H), 3.84 (s, 2H), 3.74 (s, 3H), 3.40 (q, 2H), 3.33 (t, 2H), 2.86 (t, 2H), 2.29 (s, 3H), 2.09 (s, 3H), 1.98 (qn, 2H), 1.39 (s, 2H), 1.27/1.21 (d+d, 4H), 1.18/1.12 (d+d, 4H), 1.03/0.94 (d+d, 2H), 0.84 (s, 6H); ¹³C NMR (125 MHz, DMSO-d₆) δ ppm 166.8, 159.7, 156.3, 153.6, 150.8, 147.8, 140.2, 137.6, 137.3, 136.0, 130.2, 129.1, 127.7, 123.0, 120.4, 115.6, 114.3, 74.0, 66.8, 62.2, 61.5, 59.0, 55.6, 50.0, 46.9, 46.0, 46.0, 43.3, 39.7, 33.5, 30.1, 24.6, 21.0, 15.5, 10.9. HRMS-ESI (m/z): [M+H]⁺ calcd for C₄₁H₅₀ClN₆O₅: 741.3531. found: 741.3530.

### Step F: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3-(2-hydroxyethoxy)-5,7-dimethyl-1-adamantyl]methyl]-5-methyl-pyrazol-4-yl]pyridine-2-carboxylate

The mixture of the product of Step E (4.70 g, 6.30 mmol), 1,3-benzothiazol-2-amine (1.90 g, 2 eq), DIPEA (3.30 mL, 3 eq), Pd₂(dba)₃ (580 mg, 0.1 eq), and XantPhos (730 mg, 0.2 eq) in cyclohexanol (38 mL) was kept at 130° C. for 2 h. Then, the product was purified by column chromatography (silica gel, heptane, EtOAc, and MeOH as eluents) to give the desired product (3.83 g, 71%). ¹H NMR (500 MHz, DMSO-d₆) δ ppm 7.95 (d, 1H), 7.81 (br d, 1H), 7.69 (d, 1H), 7.49 (br s, 1H), 7.39 (s, 1H), 7.35 (m, 1H), 7.19 (m, 2H), 7.16 (m, 1H), 6.91 (m, 2H), 5.10 (s, 2H), 4.46 (t, 1H), 3.99 (m, 2H), 3.85 (s, 2H), 3.75 (s, 3H), 3.40 (m, 2H), 3.34 (t, 2H), 2.85 (t, 2H), 2.32 (s, 3H), 2.11 (s, 3H), 1.99 (m, 2H), 1.45-0.90 (m, 12H), 0.84 (s, 6H); HRMS-ESI (m/z): [M+H]⁺ calcd for C₄₈H₅₅N₈O₅S: 855.4016. found: 855.4011.

### Step G: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3,5-dimethyl-7-[2-(p-tolylsulfonyl oxy)ethoxy]-1-adamantyl]methyl]-5-methyl-pyrazol-4-yl]pyridine-2-carboxylate

To the product of Step F (3.83 g, 4.48 mmol) and triethylamine (1.87 mL, 3 eq) in DCM (45 mL) was added p-tolylsulfonyl 4-methylbenzenesulfonate (2.19 g, 1.5 eq) at 0° C. and the mixture was stirred at RT for 2 h. Crude product was purified by column chromatography (silica gel, heptane and EtOAc, as eluents) to give the desired product (2.50 g, 55%). ¹H NMR (500 MHz, DMSO-d₆) δ ppm 7.95 (d, 1H), 7.81 (br s, 1H), 7.76 (m, 2H), 7.45 (br s, 1H), 7.45 (m, 2H), 7.40 (s, 1H), 7.35 (m, 1H), 7.18 (m, 2H), 7.17 (m, 1H), 6.97 (d, 1H), 6.90 (m, 2H), 5.10 (s, 2H), 4.05 (m, 2H), 4.00 (m, 2H), 3.82 (s, 2H), 3.74 (s, 3H), 3.47 (m, 2H), 2.85 (m, 2H), 2.40 (s, 3H), 2.32 (s, 3H), 2.10 (s, 3H), 1.98 (m, 2H), 1.87-1.34 (m, 12H), 0.81 (s, 6H); HRMS-ESI (m/z): [M+H]⁺ calcd for C₅₅H₆₁N₈O₇S₂: 1009.4105. found: 1009.4102.

### Step H: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3,5-dimethyl-7-[2-(methylamino) ethoxy]-1-adamantyl]methyl]-5-methyl-pyrazol-4-yl]pyridine-2-carboxylate

To the product of Step G (2.00 g, 1.98 mmol) in MeCN (9.9 mL) was added a 2 M methanamine solution in THF (9.9 mL, 10 eq) and the mixture was stirred at 50° C. for 18 h in a closed bottle. The crude product was purified by column chromatography (silica gel, using heptane, EtOAc, and MeOH as eluents) and preparative HPLC (Interchim Method) (C18, 5 mM aqeuous NH₄HCO₃, MeCN) to give the desired product (922 mg, 54%). ¹H NMR (500 MHz, DMSO-d₆) δ ppm 7.94 (d, 1H), 7.81 (dm, 1H), 7.68 (d, 1H), 7.50 (brd, 1H), 7.39 (s, 1H), 7.35 (m, 1H), 7.19 (m, 2H), 7.16 (m, 1H), 6.94 (m, 2H), 5.10 (s, 2H), 3.99 (m, 2H), 3.85 (s, 2H), 3.75 (s, 3H), 3.37 (t, 2H), 2.85 (t, 2H), 2.51 (t, 2H), 2.32 (s, 3H), 2.25 (s, 3H), 2.11 (s, 3H), 1.98 (m, 2H), 1.44-0.90 (m, 12H), 0.84 (s, 6H); ¹³C NMR (125 MHz, DMSO-d₆) δ ppm 140.0, 137.7, 130.2, 126.4, 122.3, 122.0, 118.9, 116.9, 114.3, 60.7, 59.4, 59.0, 55.6, 52.2, 45.4, 36.5, 30.1, 24.3, 21.7, 12.6, 10.9; HRMS-ESI (m/z): [M+H]⁺ calcd for C₄₉H₅₈N₉O₄S: 868.4332. found: 868.4329.

### Preparation 5: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-piperazin-1-ylethoxy)-1-adamantyl]methyl]-5-methyl-pyrazol-4-yl]pyridine-2-carboxylic acid

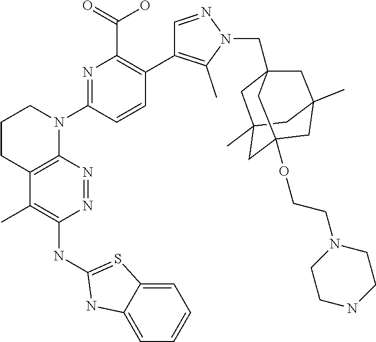

After stirring the product of Step G of Preparation 4 (570 mg, 0.61 mmol) and piperazine (440 mg, 8.4 eq) in MeCN (10.0 mL) at 50° C. for 18 h, 10% aqueous KOH solution (2.0 mL) was added and stirred at 50° C. for 4 hours. The crude product was purified by column chromatography (silica gel, using heptane, EtOAc, and MeOH as eluents) and preparative HPLC (Interchim Method) (C18, 5 mM aqeuous NH₄HCO₃, MeCN) to give the desired product (247 mg, 51%). ¹H NMR (500 MHz, dmso-d6) δ ppm 7.80 (brd, 1H), 7.61 (d, 1H), 7.50 (brs, 1H), 7.50 (s, 1H), 7.46 (d, 1H), 7.34 (m, 1H), 7.16 (m, 1H), 3.96 (m, 2H), 3.85 (s, 2H), 3.49 (t, 2H), 2.95 (m, 4H), 2.85 (t, 2H), 2.73 (m, 4H), 2.71 (t, 2H), 2.30 (s, 3H), 2.27 (s, 3H), 1.97 (m, 2H), 1.41-1.03 (m, 12H), 0.88 (s, 6H); ¹³C NMR (125 MHz, dmso-d6) δ ppm 138.8, 138.3, 126.4, 122.3, 122.0, 115.6, 59.0, 57.3, 57.0, 49.6, 45.6, 43.2, 30.1, 24.5, 21.7, 12.5, 11.5; HRMS-ESI (m/z): [M+H]⁺ calcd for C₄₄H₅₅N₁₀O₃S: 803.4179. found: 803.4177.

### Preparation 6: (Z)—N-(6-chloro-4-methyl-pyridazin-3-yl)-3-(2-trimethylsilylethoxy methyl)-1,3-benzothiazol-2-imine

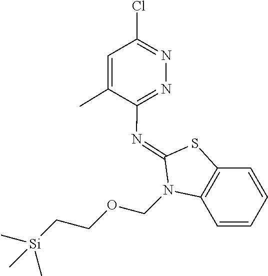

### Step A: N-(6-chloro-4-methyl-pyridazin-3-yl)-1,3-benzothiazol-2-amine

After stirring 6-chloro-4-methyl-pyridazin-3-amine (34.0 g, 236 mmol), 2-chloro-1,3-benzothiazole (44.1 g, 1.1 eq), diisopropylethylamine (123 mL, 3 eq), Cs₂CO₃ (137 g, 3 eq), Pd₂(dba)₃ (2.0 g, 0.025 eq), and XantPhos (6.8 g, 0.05 eq) in DMF (1 L) at 75° C. for 4 h, the reaction mixture was diluted with water, and the crude product was filtered off. Desired product (68.4 g, 104%) was obtained after triturating in heptane-Et₂O (3:2). ¹H NMR (500 MHz, DMSO-d₆) δ ppm 11.96 (br s, 1H), 7.86 (d, 1H), 7.65 (s, 1H), 7.51 (d, 1H), 7.38 (t, 1H), 7.21 (t, 1H), 2.37 (s, 3H); ¹³C NMR (125 MHz, DMSO-d₆) δ ppm 130.3, 129.5, 126.6, 122.8, 122.3, 17.2; HRMS-ESI (m/z): [M+H]⁺ calcd for C₁₂H₁₀ClN₄S: 277.0315. found: 277.0305.

### Step B: (Z)—N-(6-chloro-4-methyl-pyridazin-3-yl)-3-(2-trimethylsilylethoxymethyl)-1,3-benzothiazol-2-imine

To the product of Step A (68.4 g, 247 mmol) and diisopropylethylamine (129 mL, 3 eq) in DCM (1 L) was added 2-(chloromethoxy)ethyl-trimethyl-silane (48.1 mL, 1.1 eq) at 0° C. and the mixture was stirred at RT for 15 min. The reaction was quenched with the addition of water and the product was purified by column chromatography (silica gel, heptane and EtOAc as eluents) to give the desired product (42.1 g, 42%). ¹H NMR (500 MHz, DMSO-d₆) δ ppm 7.85 (dm, 1H), 7.72 (q, 1H), 7.53 (dm, 1H), 7.47 (m, 1H), 7.29 (m, 1H), 5.89 (s, 2H), 3.70 (m, 2H), 2.39 (d, 3H), 0.90 (m, 2H), −0.12 (s, 9H); ¹³C NMR (125 MHz, DMSO-d₆) δ ppm 159.5, 158.5, 150.0, 138.1, 137.4, 129.5, 127.4, 125.5, 123.8, 123.2, 112.4, 73.0, 66.8, 17.7, 17.1, −1.0; HRMS-ESI (m/z): [M+H]⁺ calcd for C₁₅H₂₄ClN₄OSSi: 407.1129. found: 407.1120.

### Preparation 7: (4-methoxyphenyl)methyl 6-[5-azidopentyl-[5-methyl-6-[(Z)-[3-(2-trimethylsilylethoxymethyl)-1,3-benzothiazol-2-ylidene]amino]pyridazin-3-yl]amino]-3-[1-[[3-[2-(dimethylamino)ethoxy]-5,7-dimethyl-1-adamantyl]methyl]-5-methyl-pyrazol-4-yl]pyridine-2-carboxylate

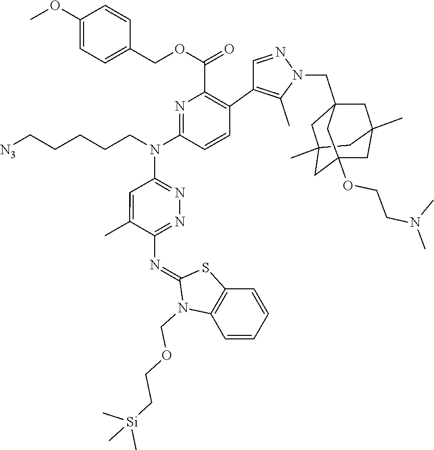

### Step A: methyl 3-bromo-6-[tert-butoxycarbonyl(5-chloropentyl)amino]pyridine-2-carboxylate

After stirring the product of Step B of Preparation 2 (4.48 g, 13.5 mmol), 1-chloro-5-iodo-pentane (1.89 mL, 1 eq), and Cs₂CO₃ (13.2 g, 3 eq) in acetone (68 mL) for 18 h, the reaction was quenched with the addition of water, and extracted with EtOAc. After concentration of the organic phases, the crude product was purified by column chromatography (silica gel, using heptane, EtOAc, as eluents) to obtain 4.89 g of the desired product (83%). ¹H NMR (500 MHz, DMSO-d₆) δ ppm 8.13 (d, 1H), 7.73 (d, 1H), 3.89 (s, 3H), 3.82 (t, 2H), 3.61 (t, 2H), 1.71 (m, 2H), 1.57 (m, 2H), 1.47 (s, 9H), 1.36 (m, 2H); ¹³C NMR (125 MHz, DMSO-d₆) δ ppm 143.1, 123.1, 53.4, 46.3, 45.7, 31.8, 28.2, 27.6, 23.8; HRMS (ESI) [M+H]⁺ calcd for C₁₇H₂₅BrClN₂O₄: 435.0686. found=435.0682.

### Step B: methyl 3-bromo-6-(5-chloropentylamino)pyridine-2-carboxylate

To the product of Step A (4.0 g, 9.2 mmol) in DCM (46 mL) was added TFA (4.3 mL, 6 eq) and the reaction mixture was stirred at 40° C. for 1 h. After the addition of a saturated solution of NaHCO₃ and extraction with DCM, the combined organic phases were dried and concentrated to afford the desired product (3.0 g, 97%) LC/MS (C₁₂H₁₇BrClN₂O₂) 335 [M+H]⁺.

### Step C: (4-methoxyphenyl)methyl 3-bromo-6-(5-chloropentylamino)pyridine-2-carboxylate

After stirring the product of Step B (2.86 g, 8.5 mmol), (4-methoxyphenyl)methanol (1.27 mL, 1.2 eq), La(OiPr)₃ (270 mg, 0.1 eq), 2-(2-hydroxyethoxy)ethanol (0.080 mL, 0.1 eq), and 5A molecular sieves (2.86 g) in hexane (17 mL) at 65° C. for 48 h, the desired product (2.23 g, 59%) was isolated by column chromatography (silica gel, using heptane, EtOAc, as eluents). ¹H NMR (500 MHz, DMSO-d₆) δ ppm 7.59 (d, 1H), 7.39 (dm, 2H), 7.07 (t, 1H), 6.95 (dm, 2H), 6.53 (d, 1H), 5.25 (s, 2H), 3.75 (s, 3H), 3.61 (t, 2H), 3.16 (q, 2H), 1.71 (m, 2H), 1.51 (m, 2H), 1.41 (m, 2H); ¹³C NMR (125 MHz, DMSO-d₆) δ ppm 166.0, 159.8, 157.6, 148.1, 141.3, 130.6, 127.9, 114.3, 112.4, 101.2, 67.0, 55.6, 45.8, 40.9, 32.2, 28.3, 24.3; HRMS (ESI) [M+H]⁺ calcd for C₁₉H₂₃BrClN₂O₃: 441.0581. found=441.0577.

### Step D: (4-methoxyphenyl)methyl 3-[1-[[3-[2-[tert-butyl(diphenyl)silyl]oxyethoxy]-5,7-dimethyl-1-adamantyl]methyl]-5-methyl-pyrazol-4-yl]-6-(5-chloropentylamino) pyridine-2-carboxylate

The mixture of the product of Step C (2.00 g, 4.53 mmol), Preparation 3 (4.02 g, 1.3 eq), Cs₂CO₃ (4.43 g, 3 eq), and Pd(AtaPhos)₂Cl₂ (200 mg, 0.1 eq) in 1,4-dioxane (27 mL) and H₂O (4.5 mL) was stirred at 80° C. for 1.5 h. Purification by column chromatography (silica gel, heptane and EtOAc as eluents) afforded the desired product (4.0 g, 96%). ¹H NMR (500 MHz, DMSO-d₆) δ ppm 7.69-7.35 (m, 10H), 7.28 (d, 1H), 7.25 (s, 1H), 7.11 (dm, 2H), 6.86 (dm, 2H), 6.59 (d, 1H), 4.99 (s, 2H), 3.79 (s, 2H), 3.71 (s, 3H), 3.66 (t, 2H), 3.62 (t, 2H), 3.44 (t, 2H), 3.21 (q, 2H), 2.01 (s, 3H), 1.74 (m, 2H), 1.54 (m, 2H), 1.45 (m, 2H), 1.38-1.06 (m, 12H), 0.97 (s, 9H), 0.82 (s, 6H); ¹³C NMR (125 MHz, DMSO-d₆) δ ppm 140.2, 137.6, 130.1, 114.2, 109.9, 66.3, 64.4, 61.7, 59.0, 55.5, 45.9, 41.0, 32.3, 30.1, 28.6, 27.1, 24.4, 10.8; HRMS (ESI) [M+H]⁺ calcd for C₅₄H₇₀ClN₄O₅Si: 917.4804. found 917.4803.

### Step E: (4-methoxyphenyl)methyl 3-[1-[[3-[2-[tert-butyl(diphenyl)silyl]oxyethoxy]-5,7-dimethyl-1-adamantyl]methyl]-5-methyl-pyrazol-4-yl]-6-[5-chloropentyl-[5-methyl-6-[(Z)-[3-(2-trimethylsilylethoxymethyl)-1,3-benzothiazol-2-ylidene]amino] pyridazin-3-yl]amino]pyridine-2-carboxylate

The mixture of the product of Step D (3.23 g, 3.52 mmol), the product of Preparation 6 (2.15 g, 5.28 mmol, 1.5 eq), diisopropylethylamine (1.84 mL, 3 eq), Pd₂(dba)₃ (322 mg, 0.1 eq), and XantPhos (407 mg, 0.2 eq) in 1,4-dioxane (17 mL) was stirred at 120° C. for 3 h. After quenching with brine and extracting with EtOAc, the organic phases were dried, concentrated and purified by column chromatography (silica gel, heptane and EtOAc as eluents) to give the desired product (3.81 g, 84%). ¹H NMR (500 MHz, DMSO-d₆) δ ppm 7.78 (d, 1H), 7.68-7.35 (m, 10H), 7.58 (d, 1H), 7.54 (s, 1H), 7.47 (d, 1H), 7.45 (t, 1H), 7.37 (s, 1H), 7.25 (t, 1H), 7.17 (d, 1H), 7.15 (dm, 2H), 6.85 (dm, 2H), 5.87 (s, 2H), 5.07 (s, 2H), 4.18 (t, 2H), 3.83 (s, 2H), 3.72 (t, 2H), 3.70 (s, 3H), 3.66 (t, 2H), 3.58 (t, 2H), 3.45 (t, 2H), 2.31 (s, 3H), 2.08 (s, 3H), 1.73 (m, 2H), 1.70 (m, 2H), 1.43 (m, 2H), 1.39-0.91 (m, 12H), 0.96 (s, 9H), 0.91 (t, 2H), 0.83 (s, 6H), −0.11 (s, 9H); ¹³C NMR (125 MHz, DMSO-d₆) δ ppm 141.3, 137.6, 130.0, 127.2, 124.1, 123.4, 123.1, 114.4, 114.2, 112.0, 72.9, 66.7, 66.6, 64.4, 61.7, 59.0, 55.5, 48.2, 45.8, 32.2, 30.1, 27.3, 27.1, 24.1, 17.8, 17.3, 10.9, 0.9; HRMS (ESI) [M+H]⁺ calcd for C₇₂H₉₂ClN₈O₆SSi₂: 1287.6088. found 1287.6084.

### Step F: (4-methoxyphenyl)methyl 6-[5-azidopentyl-[5-methyl-6-[(Z)-[3-(2-trimethyl silylethoxymethyl)-1,3-benzothiazol-2-ylidene]amino]pyridazin-3-yl]amino]-3-[1-[[3-[2-[tert-butyl(diphenyl)silyl]oxyethoxy]-5,7-dimethyl-1-adamantyl]methyl]-5-methyl-pyrazol-4-yl]pyridine-2-carboxylate

The mixture of the product of Step E (3.80 g, 3 mmol) and sodium-azide (2.30 g, 12 eq) in 1-methylpyrrolidin-2-one (30 mL) was stirred at 80° C. for 18 h. After quenching with brine and extracting with EtOAc, the organic phases were dried, concentrated and purified by column chromatography (silica gel, heptane and EtOAc as eluents) to give the desired product (3.4 g, 89%). ¹H NMR (500 MHz, DMSO-d₆) δ ppm 7.78 (dm, 1H), 7.68-7.36 (m, 10H), 7.58 (d, 1H), 7.54 (s, 1H), 7.47 (dm, 1H), 7.45 (m, 1H), 7.37 (s, 1H), 7.25 (m, 1H), 7.17 (d, 1H), 7.15 (m, 2H), 6.85 (m, 2H), 5.87 (s, 2H), 5.07 (s, 2H), 4.18 (t, 2H), 3.84 (s, 2H), 3.72 (t, 2H), 3.70 (s, 3H), 3.66 (t, 2H), 3.45 (t, 2H), 3.27 (t, 2H), 2.31 (s, 3H), 2.08 (s, 3H), 1.70 (m, 2H), 1.54 (m, 2H), 1.41-0.91 (m, 12H), 1.37 (m, 2H), 0.96 (s, 9H), 0.91 (t, 2H), 0.83 (s, 6H), −0.11 (s, 9H).

### Step G: (4-methoxyphenyl)methyl 6-[5-azidopentyl-[5-methyl-6-[(Z)-[3-(2-trimethyl silylethoxymethyl)-1,3-benzothiazol-2-ylidene]amino]pyridazin-3-yl]amino]-3-[1-[[3-(2-hydroxyethoxy)-5,7-dimethyl-1-adamantyl]methyl]-5-methyl-pyrazol-4-yl] pyridine-2-carboxylate

To the product of Step F (3.40 g, 2.62 mmol) in THF (27 mL) was added a 1 M solution of TBAF in THF (2.89 mL, 1.1 eq) at 0° C. and stirred for 2 h. After quenching with brine and extracting with EtOAc, the organic phases were purified by column chromatography (silica gel, heptane and EtOAc as eluents) to give the desired product (2.34 g, 84%). ¹H NMR (500 MHz, DMSO-d₆) δ ppm 7.79 (dm, 1H), 7.62 (d, 1H), 7.55 (s, 1H), 7.47 (dm, 1H), 7.43 (m, 1H), 7.37 (s, 1H), 7.25 (m, 1H), 7.20 (d, 1H), 7.18 (m, 2H), 6.88 (m, 2H), 5.87 (s, 2H), 5.09 (s, 2H), 4.45 (t, 1H), 4.18 (t, 2H), 3.84 (s, 2H), 3.72 (s, 3H), 3.72 (t, 2H), 3.40 (m, 2H), 3.34 (t, 2H), 3.27 (t, 2H), 2.31 (s, 3H), 2.10 (s, 3H), 1.70 (m, 2H), 1.55 (m, 2H), 1.44-0.90 (m, 12H), 1.37 (m, 2H), 0.92 (t, 2H), 0.84 (s, 6H), −0.11 (s, 9H); HRMS (ESI) [M+H]⁺ calcd for C₅₆H₇₄N₁₁O₆SSi: 1056.5314. found 1056.5322.

### Step H: (4-methoxyphenyl)methyl 6-[5-azidopentyl-[5-methyl-6-[(Z)-[3-(2-trimethyl silylethoxymethyl)-1,3-benzothiazol-2-ylidene]amino]pyridazin-3-yl]amino]-3-[1-[[3-[2-(dimethylamino)ethoxy]-5,7-dimethyl-1-adamantyl]methyl]-5-methyl-pyrazol-4-yl]pyridine-2-carboxylate

To the product of Step G (2.34 g, 2.2 mmol) and triethylamine (1.24 mL, 4 eq) in DCM (22 mL) was added p-tolylsulfonyl 4-methylbenzenesulfonate (1.44 g, 2 eq). After 15 min stirring, volatiles were removed under reduced pressure, the residue was treated with MeCN (11 mL) and a 2M solution of dimethylamine in THF (11 mL, 10 eq), and stirred at 40° C. for 5 h. After quenching with a saturated solution of NH₄Cl and extracting with EtOAc, the organic phases were dried, concentrated and purified by preparative HPLC (Interchim Method) (C18, 5 mM aqeuous NH₄HCO₃, IPA) to give the desired product (1.1 g, 45%). ¹H NMR (500 MHz, DMSO-d₆) δ ppm 7.79 (d, 1H), 7.61 (d, 1H), 7.54 (s, 1H), 7.47 (d, 1H), 7.43 (td, 1H), 7.37 (s, 1H), 7.25 (td, 1H), 7.19 (d, 1H), 7.18 (dm, 2H), 6.88 (dm, 2H), 5.87 (s, 2H), 5.09 (s, 2H), 4.16 (t, 2H), 3.85 (s, 2H), 3.72 (s, 3H), 3.72 (t, 2H), 3.39 (t, 2H), 3.27 (t, 2H), 2.32 (s, 3H), 2.29 (t, 2H), 2.11 (s, 6H), 2.10 (s, 3H), 1.70 (m, 2H), 1.55 (m, 2H), 1.42-0.91 (m, 12H), 1.38 (m, 2H), 0.92 (t, 2H), 0.86 (s, 6H), −0.11 (s, 9H); ¹³C NMR (125 MHz, DMSO-d₆) δ ppm 141.3, 137.7, 129.9, 127.2, 124.1, 123.4, 123.1, 114.4, 114.2, 112.0, 72.9, 66.7, 66.5, 59.8, 59.0, 58.6, 55.5, 51.0, 48.2, 46.1, 30.1, 28.4, 27.6, 23.9, 17.8, 17.3, 10.9, −0.9; HRMS (ESI) [M+H]⁺ calcd for C₅₈H₇₉N₁₂O₅SSi: 1083.5786. found 1083.5784.

### Preparation 8: 2-[[6-(1,3-benzothiazol-2-ylamino)-5-methyl-pyridazin-3-yl]-pent-4-ynyl-amino]-5-[3-[2-fluoro-4-[3-(methylamino)prop-1-ynyl]phenoxy]propyl]thiazole-4-carboxylic acid

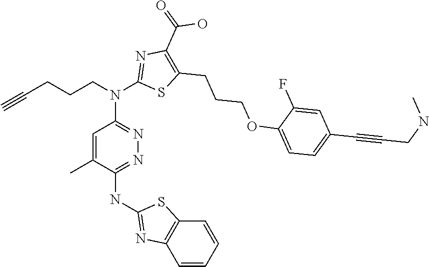

### Step A: methyl 2-(tert-butoxycarbonylamino)-5-iodo-thiazole-4-carboxylate

To 50.00 g of methyl 2-(tert-butoxycarbonylamino)thiazole-4-carboxylate (193.55 mmol) in 600 mL of dry MeCN was added 52.25 g of N-iodo succinimide (232.30 mmol) and the resulting mixture was stirred for 18 h. The reaction mixture was diluted with brine and extracted with EtOAc. The combined organic layers were extracted with 1 M solution of Na₂S₂O₃ and brine, dried, concentrated and purified by flash chromatography (silica gel, using heptane as eluent) to give the desired product (60 g, 80%). ¹H NMR (400 MHz, DMSO-d₆): δ ppm 12.03/11.06 (br s, 1H), 3.78 (s, 3H), 1.47 (s, 9H); ¹³C NMR (100 MHz, DMSO-d₆) δ ppm 153.8, 82.5, 77.7, 52.3, 28.3; HRMS-ESI (m/z): [M+H]⁺ calcd for C₁₀H₁₄IN₂O₄S: 384.9713. found 384.9708.

### Step B: methyl 2-(tert-butoxycarbonylamino)-5-(3-hydroxyprop-1-ynyl)thiazole-4-carboxylate

The mixture of 9.6 g of the product from Step A (25 mmol), 2.91 mL of prop-2-yn-1-ol (2 eq), 50 mL of diisopropylamine (14.27 eq), 549 mg of Pd(PPh₃)₂Cl₂ (0.05 eq), and 238 mg of CuI (0.05 eq) in 125 mL of THF was stirred at 60° C. until no further conversion was observed. The product was purified via flash chromatography using heptane and EtOAc as eluents to give 7.30 g of the desired product (93%). ¹H NMR (400 MHz, DMSO-d₆): δ ppm 12.10 (br s, 1H), 5.45 (t, 1H), 4.36 (d, 2H), 3.79 (s, 3H), 1.48 (s, 9H); ¹³C NMR (100 MHz, DMSO-d₆) δ ppm 12.1 (br s, 1H), 5.45 (t, 1H), 4.36 (d, 2H), 3.79 (s, 3H), 1.48 (s, 9H); HRMS-ESI (m/z): [M+H]⁺ calcd for C₁₃H₁₇N₂O₅S: 313.0852. found 313.0866.

### Step C: methyl 2-(tert-butoxycarbonylamino)-5-(3-hydroxypropyl)thiazole-4-carboxylate

The mixture of 44.75 g of the product from Step B (143.3 mmol) and 7.62 g of Pd/C (0.05 eq) in 340 mL of ethanol under 4 bar of H₂ gas was stirred for 18 h. After filtration through a pad of Celite, the mixture was treated with 7.62 g of Pd/C (0.05 eq) and stirred under 4 bar of H₂ gas for 18 h. The product was purified via flash chromatography column using heptane and EtOAc as eluents to give 31.9 g of the desired product (70.4%). ¹H NMR (500 MHz, DMSO-d₆): δ ppm 11.61 (br s, 1H), 4.54 (t, 1H), 3.76 (s, 3H), 3.43 (m, 2H), 3.09 (t, 2H), 1.74 (m, 2H), 1.46 (s, 9H); ¹³C NMR (125 MHz, DMSO-d₆) δ ppm 162.8, 143.1, 135.4, 60.3, 51.9, 34.5, 28.3, 23.4; HRMS-ESI (m/z): [M+H]⁺ calcd for C₁₃H₂₁N₂O₅S: 317.1165. found 317.1164.

### Step D: methyl 2-(tert-butoxycarbonylamino)-5-[3-(2-fluoro-4-iodo-phenoxy)propyl]thiazole-4-carboxylate

To the mixture of 3.40 g of 2-fluoro-4-iodo-phenol (14 mmol), 5.00 g of the product from Step C (16 mmol, 1.1 eq), and 4.10 g of PPh₃ (1.1 eq) in 71 mL of toluene was added 3.10 mL of DIAD (3.20 g, 1.1 eq). After stirring at 50° C. for 30 min, the reaction mixture was directly injected onto a preconditioned silica gel column, and then it was purified via flash chromatography using heptane and EtOAc as eluents. The crude product was crystalized from MeOH to give 4.64 g of the desired product (66%).

¹H NMR (500 MHz, DMSO-d₆) δ ppm 11.64 (br s, 1H), 7.59 (dd, 1H), 7.45 (dd, 1H), 6.98 (t, 1H), 4.06 (t, 2H), 3.73 (s, 3H), 3.22 (t, 2H), 2.06 (m, 2H), 1.46 (s, 9H); ¹³C NMR (125 MHz, DMSO-d₆) δ ppm 134.0, 124.9, 117.6, 68.2, 51.9, 30.5, 28.3, 23.2; HRMS-ESI (m/z): [M+H]⁺ calcd for C₁₉H₂₃N₂O₅FSI: 537.0350. found 537.0348.

### Step E: methyl 2-[tert-butoxycarbonyl(5-trimethylsilylpent-4-ynyl)amino]-5-[3-(2-fluoro-4-iodo-phenoxy)propyl]thiazole-4-carboxylate

After mixing the product of Step D (5.36 g, 10 mmol, 1 eq) and 5-trimethylsilyl pent-4-yn-1-ol (3.12 g, 20 mmol, 2 eq) in toluene (50 mL) with PPh₃ (5.24 g, 2 eq), DIAD (3.9 mL, 2 eq) was added, and stirred at 50° C. for 30 min. Purification by column chromatography (silica gel, heptane and EtOAc as eluents) afforded the desired product (6.0 g, 89%). MS-ESI (m/z): 675 [M+H]⁺.

### Step F: methyl 5-[3-(2-fluoro-4-iodo-phenoxy)propyl]-2-(5-trimethylsilylpent-4-ynylamino)thiazole-4-carboxylate

After stirring the product of Step E (6.00 g, 8.9 mmol) in hexafluoroisopropanol (44 mL) at 100° C. for 5 hours, volatiles were removed under reduced pressure and the crude product was purified by column chromatography (silica gel, heptane and EtOAc as eluents) to give the desired product (4.46 g, 87%). ¹H NMR (500 MHz, DMSO-d₆) δ ppm 7.61 (t, 1H), 7.59 (dd, 1H), 7.45 (dm, 1H), 6.97 (t, 1H), 4.03 (t, 2H), 3.69 (s, 3H), 3.22 (m, 2H), 3.11 (t, 2H), 2.28 (t, 2H), 1.99 (m, 2H), 1.68 (m, 2H), 0.11 (s, 9H); ¹³C NMR (500 MHz, DMSO-d₆) δ ppm 134.0, 124.9, 117.6, 107.8, 85.1, 68.1, 51.7, 43.5, 30.6, 28.0, 23.3, 17.1, 0.6; HRMS-ESI (m/z): [M+H]⁺ calcd for C₂₂H₂₉FIN₂O₃SSi: 575.0697. found 575.0695.

### Step G: methyl 5-[3-[4-[3-[tert-butoxycarbonyl(methyl)amino]prop-1-ynyl]-2-fluoro-phenoxy]propyl]-2-(5-trimethylsilylpent-4-ynylamino)thiazole-4-carboxylate

To the mixture of the product of Step F (4.46 g, 7.7 mmol), Pd(PPh₃)₂Cl₂ (272 mg, 0.05 eq), and CuI (74 mg, 0.05 eq) in diisopropylamine (15 mL) and THF (30 mL) was added tert-butyl N-methyl-N-prop-2-ynyl-carbamate (2.62 g, 2 eq) and stirred at 60° C. for 1 h. Purification by column chromatography (silica gel, heptane and EtOAc as eluents) afforded the desired product (4.44 g, 93%). ¹H NMR (500 MHz, DMSO-d₆) δ ppm 7.61 (t, 1H), 7.30 (br d, 1H), 7.21 (dm, 1H), 7.12 (t, 1H), 4.23 (br s, 2H), 4.07 (t, 2H), 3.69 (s, 3H), 3.22 (m, 2H), 3.12 (t, 2H), 2.87 (br s, 3H), 2.28 (t, 2H), 2 (m, 2H), 1.68 (m, 2H), 1.41 (s, 9H), 0.11 (s, 9H); HRMS-ESI (m/z): [M+H]⁺ calcd for C₃₁H₄₃FN₃O₅SSi: 616.2677. found 616.2659.

### Step H: methyl 5-[3-[4-[3-[tert-butoxycarbonyl(methyl)amino]prop-1-ynyl]-2-fluoro-phenoxy]propyl]-2-[[5-methyl-6-[(Z)-[3-(2-trimethylsilylethoxymethyl)-1,3-benzo thiazol-2-ylidene]amino]pyridazin-3-yl]-(5-trimethylsilylpent-4-ynyl)amino]thiazole-4-carboxylate

The mixture of the product of Step G (4.44 g, 7.21 mmol), the product of Preparation 6 (3.52 g, 1.2 eq), diisopropylethylamine (3.77 mL, 3 eq), Pd₂(dba)₃ (660 mg, 0.1 eq), and XantPhos (834 mg, 0.2 eq) in 1,4-dioxane (36 mL) was stirred at 120° C. for 1 h. Purification by column chromatography (silica gel, heptane and EtOAc as eluents) afforded the desired product (3.85 g, 54%). ¹H NMR (500 MHz, DMSO-d₆) δ ppm 7.83 (d, 1H), 7.64 (s, 1H), 7.45 (dd, 1H), 7.42 (td, 1H), 7.31 (brd., 1H), 7.24 (td, 1H), 7.21 (d, 1H), 7.15 (t, 1H), 5.85 (s, 2H), 4.37 (t, 2H), 4.2 (br., 2H), 4.14 (t, 2H), 3.77 (s, 3H), 3.71 (t, 2H), 3.25 (t, 2H), 2.84 (br., 3H), 2.44 (s, 3H), 2.37 (t, 2H), 2.12 (m, 2H), 1.91 (m, 2H), 1.40 (s, 9H), 0.91 (t, 2H), 0.09 (s, 9H), −0.12 (s, 9H); ¹³C NMR (125 MHz, DMSO-d₆) δ ppm 163.1, 157.5, 155.2, 150.9, 137.6, 129.1, 127.2, 125.4, 123.4, 123.2, 119.3, 117.4, 115.4, 111.9, 107.5, 85.2, 72.9, 68.4, 66.7, 52.0, 46.5, 38.6, 33.8, 31, 28.5, 26.2, 23.2, 17.9, 17.8, 17.1, 0.5, −1.0; HRMS-ESI (m/z): [M+H]⁺ calcd for C₄₉H₆₅FN₇O₆S₂Si₂: 986.3960. found 986.3932.

### Step I: methyl 2-[[6-(1,3-benzothiazol-2-ylamino)-5-methyl-pyridazin-3-yl]-pent-4-ynyl-amino]-5-[3-[2-fluoro-4-[3-(methylamino)prop-1-ynyl]phenoxy]propyl] thiazole-4-carboxylate

The product of Step H (350 mg, 0.35 mmol) in MeCN (3.5 mL) was treated with 70% HF in pyridine (4.57 mL, 100 eq) and stirred at 60° C. for 2 h. After quenching the reaction with a 6 M NH₃ solution in MeOH, the product was purified by column chromatography (silica gel, heptane and EtOAc as eluents) to give the desired product (160 mg, 66%). MS-ESI (m/z): 684 [M+H]⁺.

### Step J: 2-[[6-(1,3-benzothiazol-2-ylamino)-5-methyl-pyridazin-3-yl]-pent-4-ynyl-amino]-5-[3-[2-fluoro-4-[3-(methylamino)prop-1-ynyl]phenoxy]propyl]thiazole-4-carboxylic acid

The suspension of the product of Step/(160 mg, 0.23 mmol) in THF (2.34 mL) and water (0.47 mL) was treated with LiOH×H₂O (19 mg, 2 eq), stirred at 60° C. for 2 h, and purified by preparative HPLC (Interchim Method) (C18, 25 mM aqeuous NH₄HCO₃, MeCN) to give the desired product (30 mg, 19%). ¹H NMR (500 MHz, DMSO-d₆) δ ppm 7.91 (dm, 1H), 7.69 (s, 1H), 7.53 (dm, 1H), 7.39 (m, 1H), 7.34 (dd, 1H), 7.25 (dm, 1H), 7.21 (m, 1H), 7.19 (t, 1H), 4.38 (t, 2H), 4.16 (t, 2H), 3.87 (s, 2H), 3.27 (t, 2H), 2.88 (t, 1H), 2.51 (s, 3H), 2.46 (s, 3H), 2.31 (m, 2H), 2.14 (m, 2H), 1.91 (m, 2H); HRMS-ESI (m/z): [M+H]⁺ calcd for C₃₄H₃₃FN₇O₃S₂: 670.2070. found 670.2052.

### Preparation 9: 2-[3-(1,3-Benzothiazol-2-ylamino)-4-methyl-6,7-dihydro-5H-pyrido[2,3-c]pyridazin-8-yl]-5-[3-[2-fluoro-4-(3-piperazin-1-ylprop-1-ynyl)phenoxy] propyl]thiazole-4-carboxylic acid

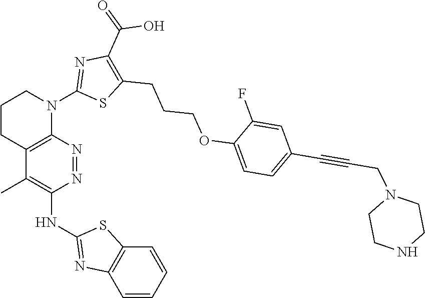 [SMILES: Cc1c(Nc2nc3ccccc3s2)nnc2c1CCCN2c1nc(C(=O)O)c(CCCOc2ccc(C#CCN3CCNCC3)cc2F)s1]

### Step A: methyl 2-{[(tert-butoxy)carbonyl][3-(3,6-dichloro-5-methylpyridazin-4-yl)propyl]amino}-5-[3-(2-fluoro-4-iodophenoxy)propyl]-1,3-thiazole-4-carboxylate

After mixing the product of Step D in Preparation 8 (14.5 g, 27 mmol, 1 eq), the product of Preparation 1 (6.00 g, 1 eq), and PPh₃ (7.11 g, 1 eq) in toluene (135 mL), DTAD (6.25 g, 1 eq) was added and stirred at 50° C. for 30 min. Purification by column chromatography (silica gel, heptane and EtOAc as eluents) afforded the desired product (13.1 g, 65%). ¹H NMR (500 MHz, DMSO-d₆) δ ppm 7.56 (dd, 1H), 7.44 (dm, 1H), 7.08 (m, 2H), 6.96 (t, 1H), 4.05 (t, 2H), 3.75 (s, 3H), 3.21 (t, 2H), 2.82 (m, 2H), 2.4 (s, 3H), 2.06 (m, 2H), 1.88 (m, 2H), 1.48 (s, 9H); ¹³C NMR (125 MHz, DMSO-d₆) δ ppm 162.7, 157.6, 156.7, 156.5/153.2, 152.2, 147, 142.1, 139.8, 134, 124.9, 117.6, 84, 82.4, 68.1, 52.1, 46.1, 30.4, 28.1, 27.5, 25.8, 23.1, 16.4; HRMS-ESI (m/z): [M+H]⁺ calcd for C₂₇H₃₁C₁₂FIN₄O₅S: 739.0415. found 739.0395.

### Step B: methyl 2-[3-(3,6-dichloro-5-methyl-pyridazin-4-yl)propylamino]-5-[3-(2-fluoro-4-iodo-phenoxy)propyl]thiazole-4-carboxylate

The product from Step A (15.0 g, 20 mmol) in 1,1,1,3,3,3-hexafluoroisopropanol (81 mL) was stirred at 110° C. for 18 h. After removing the volatiles under reduced pressure, purification by column chromatography (silica gel, heptane and EtOAc as eluents) afforded the desired product (8.6 g, 66%). ¹H NMR (500 MHz, DMSO-d₆) δ ppm 7.71 (t, 1H), 7.59 (dd, 1H), 7.44 (dm, 1H), 6.96 (t, 1H), 4.03 (t, 2H), 3.7 (s, 3H), 3.29 (m, 2H), 3.11 (t, 2H), 2.84 (m, 2H), 2.39 (s, 3H), 2 (m, 2H), 1.76 (m, 2H); ¹³C NMR (125 MHz, DMSO-d₆) δ ppm 164.6, 163, 152.3, 147.1, 134.1, 124.8, 117.6, 82.4, 68.1, 51.9, 44, 30.7, 28, 26.9, 23.3, 16.4; HRMS-ESI (m/z): [M+H]⁺ calcd for C₂₂H₂₃C₁₂FIN₄O₃S: 638.9891. found 638.9888.

### Step C: methyl 2-(3-chloro-4-methyl-6,7-dihydro-5H-pyrido[2,3-c]pyridazin-8-yl)-5-[3-(2-fluoro-4-iodo-phenoxy)propyl]thiazole-4-carboxylate

A suspension of 3.0 g of the product from Step B (4.69 mmol) and 1.81 g of Cs₂CO₃ (2 eq) in 25 mL of 1,4-dioxane were stirred at 80° C. for 3 h. The product was purified by flash chromatography using DCM-MeOH as eluents to give 2.67 g (94%) of the desired product. ¹H NMR (500 MHz, DMSO-d₆) δ ppm 7.57 (dd, 1H), 7.43 (dm, 1H), 6.97 (t, 1H), 4.23 (t, 2H), 4.08 (t, 2H), 3.77 (s, 3H), 3.22 (t, 2H), 2.86 (t, 2H), 2.29 (s, 3H), 2.08 (m, 2H), 2.03 (m, 2H); ¹³C NMR (125 MHz, DMSO-d₆) δ ppm 163.1, 155.4, 152.2, 151.6, 151.2, 147, 142.5, 136, 134.8, 134, 128.9, 124.9, 117.6, 82.3, 68.4, 51.9, 46.3, 30.7, 24.2, 23, 19.7, 15.7; HRMS-ESI (m/z): [M+H]⁺ calcd for C₂₂H₂₂ClFIN₄O₃S: 603.0124. found 603.0108.

### Step D: methyl 2-(3-chloro-4-methyl-6,7-dihydro-5H-pyrido[2,3-c]pyridazin-8-yl)-5-[3-[2-fluoro-4-(2-trimethylsilylethynyl)phenoxy]propyl]thiazole-4-carboxylate

To 5.0 g of the product of Step C (8.29 mmol, 1 eq.), 2.34 mL of ethynyl(trimethyl) silane (2 eq), and 10 mL DIPEA in 40 mL of THF were added 182 mg Pd(PPh₃)₂Cl₂ (0.05 eq) and 79 mg CuI (0.05 eq). The resulting mixture was stirred at 60° C. for 2 h. The product was purified by flash chromatography using Heptane-EtOAc as eluents to give 4.26 g (89%) of the desired product. ¹H NMR (500 MHz, DMSO-d₆) δ ppm 7.31 (dd, 1H), 7.23 (dn, 1H), 7.13 (t, 1H), 4.25 (t, 2H), 4.12 (t, 2H), 3.77 (s, 3H), 3.24 (t, 2H), 2.87 (t, 2H), 2.31 (s, 3H), 2.10 (m, 2H), 2.03 (m, 2H), 0.21 (s, 9H); ¹³C NMR (125 MHz, DMSO-d₆) δ ppm 163.0, 155.3, 151.7, 151.3, 136.1, 129.4, 129.0, 119.4, 115.3, 104.6, 93.7, 68.2, 51.9, 46.3, 30.7, 24.1, 23.0, 19.7, 15.7, 0.4; HRMS-ESI (m/z): [M]⁺ calcd for C₂₇H₃₀ClFN₄O₃SSi: 572.1481. found 572.1480.

### Step E: methyl 2-[3-(1,3-benzothiazol-2-ylamino)-4-methyl-6,7-dihydro-5H-pyrido[2,3-c]pyridazin-8-yl]-5-[3-[2-fluoro-4-(2-trimethylsilylethynyl) phenoxy] propyl]thiazole-4-carboxylate

To 4.25 g of the product from Step D (7.4 mmol, 1.0 eq.), 2.23 g 1,3-benzo thiazol-2-amine (2 eq), and 3.87 mL of DIPEA (3 eq) in 40 mL of cyclohexanol were added 679 mg of Pd₂(dba)₃ (0.10 eq) and 858 mg of XantPhos (0.20 eq) and the resulting mixture was stirred at 140° C. for 30 min. The product was purified by flash chromatography using heptane and EtOAc as eluents to give 3.90 g (77%) of the desired product. ¹H NMR (500 MHz, DMSO-d₆) δ ppm 12.27/10.91 (br s, 1H), 8.1-7.1 (br m, 4H), 7.34 (dd, 1H), 7.24 (dm, 1H), 7.16 (t, 1H), 4.25 (t, 2H), 4.15 (t, 2H), 3.78 (s, 3H), 3.28 (t, 2H), 2.87 (t, 2H), 2.34 (s, 3H), 2.13 (m, 2H), 2.04 (m, 2H), 0.19 (s, 9H); HRMS-ESI (m/z): [M+H]⁺ calcd for C₃₄H₃₆FN₆O₃S₂Si: 687.2038. found 687.2020.

### Step F: 2-[3-(1,3-benzothiazol-2-ylamino)-4-methyl-6,7-dihydro-5H-pyrido[2,3-c]pyridazin-8-yl]-5-[3-(4-ethynyl-2-fluoro-phenoxy)propyl]thiazole-4-carboxylic acid

The mixture of 343 mg of the product from Step E (0.5 mmol) and 105 mg of LiOH×H₂O (5 eq) in 2.5 mL of THF/H₂O (4:1) was stirred at 60° C. for 4 h. The product was purified by flash chromatography using DCM and MeOH (1.2% NH₃) as eluents to give 200 mg (66%) of the desired product. ¹H NMR (500 MHz, DMSO-d₆) δ ppm 7.88 (d, 1H), 7.49 (br s, 1H), 7.37 (t, 1H), 7.36 (dd, 1H), 7.25 (dm, 1H), 7.19 (t, 1H), 7.16 (t, 1H), 4.27 (t, 2H), 4.15 (t, 2H), 4.11 (s, 1H), 3.27 (t, 2H), 2.87 (t, 2H), 2.33 (s, 3H), 2.14 (m, 2H), 2.04 (m, 2H); ¹³C NMR (125 MHz, DMSO-d₆) δ ppm 164.2, 151.5, 147.9, 129.4, 126.5, 122.5, 122.3, 119.5, 115.5, 114.5, 82.9, 80.5, 68.5, 46.2, 31.0, 23.9, 23.1, 20.3, 12.9; HRMS-ESI (m/z): [M+H]⁺ calcd for C₃₀H₂₆FN₆O₃S₂: 601.1486. found 601.1498.

### Step G: 2-[3-(1,3-benzothiazol-2-ylamino)-4-methyl-6,7-dihydro-5H-pyrido[2,3-c]pyridazin-8-yl]-5-[3-[4-[3-(4-tert-butoxycarbonylpiperazin-1-yl)prop-1-ynyl]-2-fluoro-phenoxy]propyl]thiazole-4-carboxylic acid

The mixture of 200 mg of the product of Step F (0.33 mmol), paraformaldehyde (100 mg), CuI (63 mg) and tert-butyl piperazine-1-carboxylate (620 mg, 10 eq) in EtOH (3.3 mL) was irradiated in an Anton-Paar microwave reactor at 100° C. for 1 h. Purification by column chromatography (silica gel, heptane, EtOAc, MeOH as eluents) afforded the desired product (212 mg, 77%). ¹H NMR (500 MHz, DMSO-d₆) δ ppm 7.88 (d, 1H), 7.50 (br d, 1H), 7.45 (dd, 1H), 7.37 (t, 1H), 7.32 (d, 1H), 7.22 (t, 1H), 7.19 (t, 1H), 4.28 (t, 2H), 4.17 (t, 2H), 4.01 (br, 6H), 3.28 (t, 2H), 3.20 (br, 4H), 2.88 (t, 2H), 2.34 (s, 3H), 2.15 (qn, 2H), 2.04 (qn, 2H), 1.41 (s, 9H); ¹³C NMR (125 MHz, DMSO-d₆) δ ppm 129.6, 126.5, 122.5, 122.3, 119.7, 115.8, 115.6, 68.6, 46.3, 31.0, 28.4, 23.9, 23.1, 20.3, 12.9; HRMS-ESI (m/z): [M+H]⁺ calcd for C₄₀H₄₄FN₈O₅S₂: 799.2860. found 799.2837.

### Step H: 2-[3-(1,3-benzothiazol-2-ylamino)-4-methyl-6,7-dihydro-5H-pyrido[2,3-c]pyridazin-8-yl]-5-[3-[2-fluoro-4-(3-piperazin-1-ylprop-1-ynyl)phenoxy]propyl] thiazole-4-carboxylic acid

The mixture of the product of Step G (207 mg, 0.25 mmol) and HF×Pyr (10 eq.) in acetonitrile (4.3 mL) was stirred at 60° C. for 2.5 h. The product was purified by flash chromatography on silica gel column using DCM and MeOH as eluents to give 143 mg (79%) of the desired product. ¹H NMR (500 MHz, DMSO-d₆) δ ppm 7.87 (d, 1H), 7.49 (d, 1H), 7.37 (td, 1H), 7.30 (dd, 1H), 7.20 (d, 1H), 7.19 (td, 1H), 7.14 (t, 1H), 4.27 (t, 2H), 4.13 (t, 2H), 3.44 (s, 2H), 3.27 (t, 2H), 2.87 (t, 2H), 2.81 (br., 4H), 2.48 (br., 4H), 2.34 (s, 3H), 2.13 (m, 2H), 2.04 (m, 2H); ¹³C NMR (125 MHz, DMSO-d₆) δ ppm 129.0, 126.5, 122.5, 122.3, 119.2, 116.4, 115.5, 68.5, 51.7, 47.6, 46.3, 45.1, 31.0, 23.9, 23.0, 20.4, 12.9; HRMS-ESI (m/z): [M+H]⁺ calcd for C₃₅H₃₆FN₈O₃S₂: 699.2330. found 699.2322.

### Preparation 10: 5-[3-[4-[3-[tert-butoxycarbonyl(methyl)amino]prop-1-ynyl]-2-fluoro-phenoxy]propyl]-2-[[5-methyl-6-[(Z)-[3-(2-trimethylsilylethoxymethyl)-1,3-benzo thiazol-2-ylidene]amino]pyridazin-3-yl]-pent-4-ynyl-amino]thiazole-4-carboxylic acid

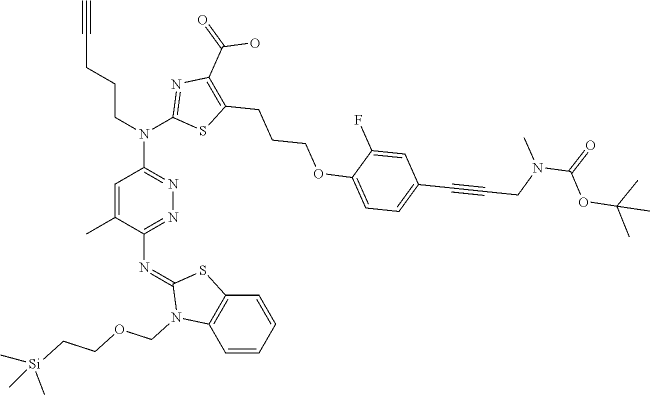

The product of Step H of Preparation 8 (1.00 g, 1.0 mmol) and LiOH×H₂O (212 mg, 5 eq) in THF (10 mL) and water (2 mL) was stirred at 40° C. for 16 h. After quenching with HCl, the mixture was extracted with DCM, concentrated and crystallized from Et₂O to give the desired product (380 mg, 41%). ¹H NMR (500 MHz, DMSO-d₆) δ ppm 7.75 (d, 1H), 7.42 (br s, 1H), 7.40 (m, 1H), 7.4 (m, 1H), 7.22 (t, 1H), 7.19 (d, 1H), 7.14 (d, 1H), 7.08 (t, 1H), 5.79 (s, 2H), 4.31 (br., 2H), 4.20 (s, 2H), 4.08 (brt., 2H), 3.73 (t, 2H), 3.21 (brt., 2H), 2.86 (s, 3H), 2.68 (brs., 1H), 2.36 (brs., 3H), 2.26 (br t, 2H), 2.05 (br., 2H), 1.88 (m, 2H), 1.42 (s, 9H), 0.90 (t, 2H), −0.10 (s, 9H); ¹³C NMR (125 MHz, DMSO-d₆) δ ppm 128.9, 127.0, 123.3, 123.0, 119.2, 118.2, 116.0, 111.7, 73.1, 71.6, 69.2, 66.8, 48.1, 38.7, 33.8, 31.1, 28.5, 26.7, 23.1, 18.0, 18.0, 15.8, −1.0.

### Preparation 11: (4-methoxyphenyl)methyl 2-[3-(1,3-benzothiazol-2-ylamino)-4-methyl-6,7-dihydro-5H-pyrido[2,3-c]pyridazin-8-yl]-5-[3-[2-fluoro-4-(3-piperazin-1-ylprop-1-ynyl)phenoxy]propyl]thiazole-4-carboxylate

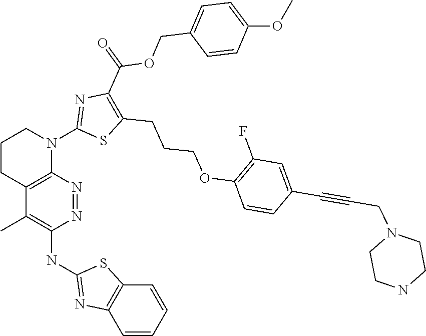

### Step A: 2-(3-chloro-4-methyl-6,7-dihydro-5H-pyrido[2,3-c]pyridazin-8-yl)-5-[3-(2-fluoro-4-iodo-phenoxy)propyl]thiazole-4-carboxylic acid

The mixture of the product of Step C of Preparation 9 (10.0 g, 16 mmol) and LiOH×H₂O (6.80 g, 10 eq) in THF (81 mL) and water (81 mL) was stirred at 50° C. for 6 h. After setting pH to 6 by the addition of HCl, desired product was filtered off (8.20 g, 86%). ¹H NMR (500 MHz, DMSO-d₆) δ ppm 7.56 (dd, 1H), 7.43 (br d, 1H), 6.96 (t, 1H), 4.18 (t, 2H), 4.05 (t, 2H), 3.28 (t, 2H), 2.84 (t, 2H), 2.29 (s, 3H), 2.07 (m, 2H), 1.97 (m, 2H); ¹³C NMR (125 MHz, DMSO-d₆) δ ppm 166.4, 154.8, 152.1, 151.8, 151.1, 147.1, 143.9, 135.7, 134.0, 133.8, 129.0, 124.9, 117.6, 82.3, 68.8, 46.3, 31.0, 24.0, 22.5, 19.8, 15.7; HRMS-ESI (m/z): [M+H]⁺ calcd for C₂₁H₂₀ClFIN₄O₃S: 588.9973. found 588.9969.

### Step B: (4-methoxyphenyl)methyl 2-(3-chloro-4-methyl-6,7-dihydro-5H-pyrido[2,3-c]pyridazin-8-yl)-5-[3-(2-fluoro-4-iodo-phenoxy)propyl]thiazole-4-carboxylate

To the mixture of the product of Step A (8.20 g, 14 mmol), PPh₃ (7.30 g, 2 eq), and (4-methoxyphenyl)methanol (3.80 g, 2 eq) in toluene (70 mL) was added DIAD (5.5 mL, 2 eq). The reaction was stirred at 50° C. for 30 min. The product was purified by column chromatography (silica gel, heptane, EtOAc as eluents) to give the desired product (8.50 g, 86%). ¹H NMR (500 MHz, DMSO-d₆) δ ppm 7.58 (dd, 1H), 7.43 (dd, 1H), 7.38 (d, 2H), 6.91 (d, 2H), 6.90 (t, 1H), 5.21 (s, 2H), 4.24 (t, 2H), 3.97 (t, 2H), 3.73 (s, 3H), 3.19 (t, 2H), 2.87 (t, 2H), 2.31 (s, 3H), 2.04 (qn, 2H), 2.02 (qn, 2H); ¹³C NMR (125 MHz, DMSO-d₆) δ ppm 162.4, 159.7, 155.4, 152.2, 151.7, 151.3, 147.0, 142.4, 136.2, 135.2, 134.0, 130.8, 129.0, 128.5, 124.9, 117.5, 114.3, 82.4, 68.3, 66.1, 55.6, 46.3, 30.8, 24.2, 23.1, 19.7, 15.7; HRMS-ESI (m/z): [M+H]⁺ calcd for C₂₉H₂₈ClFIN₄O₄S: 709.0549. found 709.0534.

### Step C: (4-methoxyphenyl)methyl 2-(3-chloro-4-methyl-6,7-dihydro-5H-pyrido[2,3-c]pyridazin-8-yl)-5-[3-[2-fluoro-4-(3-piperazin-1-ylprop-1-ynyl)phenoxy]propyl] thiazole-4-carboxylate

The mixture of the product of Step B was (7.50 g, 10.6 mmol), 1-prop-2-ynylpiperazine, hydrogen chloride (1:2) (5.00 g, 2.4 eq), Pd(PPh₃)₂Cl₂ (371 mg, 0.05 eq), and CuI (100 mg, 0.05 eq) in THF (52 mL) and DIPA (10 mL) was stirred at 60° C. for 1 h. The product was purified by column chromatography (silica gel, heptane, EtOAc and MeOH as eluents) to give the desired product (6.10 g, 82%). ¹H NMR (500 MHz, DMSO-d₆) δ ppm 8.66 (br s, 2H), 7.39 (d, 2H), 7.33 (dd, 1H), 7.21 (d, 1H), 7.08 (t, 1H), 6.92 (d, 2H), 5.22 (s, 2H), 4.24 (t, 2H), 4.02 (t, 2H), 3.73 (s, 3H), 3.57 (s, 2H), 3.21 (t, 2H), 3.08 (t, 4H), 2.88 (t, 2H), 2.71 (t, 4H), 2.31 (s, 3H), 2.06 (qn, 2H), 2.02 (qn, 2H); ¹³C NMR (125 MHz, DMSO-d₆) δ ppm 162.4, 159.7, 155.4, 151.7, 151.5, 151.3, 147.4, 142.4, 136.2, 135.1, 130.7, 129.0, 128.9, 128.5, 119.3, 115.4, 115.1, 114.3, 84.7, 84.3, 68.3, 66.1, 55.5, 48.7, 47.0, 46.3, 43.5, 30.8, 24.2, 23.1, 19.8, 15.7; HRMS-ESI (m/z): [M+H]⁺ calcd for C₃₆H₃₉ClFN₆O₄S: 705.2426. found 705.2427.

### Step D: (4-methoxyphenyl)methyl 2-[3-(1,3-benzothiazol-2-ylamino)-4-methyl-6,7-dihydro-5H-pyrido[2,3-c]pyridazin-8-yl]-5-[3-[2-fluoro-4-(3-piperazin-1-ylprop-1-ynyl)phenoxy]propyl]thiazole-4-carboxylate

The mixture of the product of Step C (5.30 g, 7.52 mmol), 1,3-benzothiazol-2-amine (3.39 g, 3 eq), DIPEA (3.9 mL, 3 eq), Pd₂(dba)₃ (344 mg, 0.05 eq) and XantPhos (435 mg, 0.1 eq) in cyclohexanol (37 mL), was stirred at 140° C. for 1 h. The product was purified by column chromatography (silica gel, heptane, EtOAc, MeOH) to give the desired compound (2.5 g, 41%). ¹H NMR (500 MHz, DMSO-d₆) δ ppm 7.87 (d, 1H), 7.87 (dd, 1H), 7.49 (d, 1H), 7.39 (d, 2H), 7.36 (t, 1H), 7.19 (dd, 1H), 7.18 (t, 1H), 7.07 (t, 1H), 6.93 (d, 2H), 5.22 (s, 2H), 4.24 (t, 2H), 4.02 (t, 2H), 3.73 (s, 3H), 3.40 (s, 2H), 3.23 (t, 2H), 2.86 (t, 2H), 2.71 (t, 4H), 2.41 (brt, 4H), 2.33 (s, 3H), 2.08 (qn, 2H), 2.03 (qn, 2H); ¹³C NMR (125 MHz, DMSO-d₆) δ ppm 130.8, 128.9, 126.5, 122.5, 122.3, 119.2, 116.4, 115.4, 114.3, 68.4, 66.0, 55.5, 52.8, 47.8, 46.3, 45.8, 31.1, 23.9, 23.2, 20.4, 12.8; HRMS-ESI (m/z): [M+H]⁺ calcd for C₄₃H₄₄FN₈O₄S₂: 819.2911. found 819.2907.

### Preparation 13: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3,5-dimethyl-7-(2-piperazin-1-ylethoxy)-1-adamantyl]methyl]-5-methyl-pyrazol-4-yl]pyridine-2-carboxylate

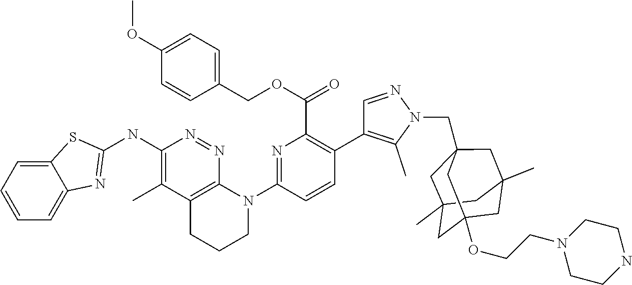 [SMILES: COc1ccc(COC(=O)c2nc(N3CCCc4c3nnc(Nc3nc5ccccc5s3)c4C)ccc2-c2cnn(CC34CC5(C)CC(C)(C3)CC(OCCN3CCNCC3)(C5)C4)c2C)cc1]

After stirring the product of Step G of Preparation 4 (100 mg, 0.1 mmol) and piperazine (85 mg, 10 eq) in MeCN (2.0 mL) at 40° C. for 8 h, the crude product was purified by preparative HPLC (Interchim Method) (C18, 5 mM aqeuous NH₄HCO₃, IPA) to give the desired product (45 mg, 49%). ¹H NMR (500 MHz, dmso-d6) 6 ppm 7.95 (d, 1H), 7.81 (dm, 1H), 7.68 (d, 1H), 7.50 (brd, 1H), 7.39 (s, 1H), 7.35 (m, 1H), 7.19 (m, 2H), 7.16 (m, 1H), 6.91 (m, 2H), 5.10 (s, 2H), 3.99 (m, 2H), 3.85 (s, 2H), 3.75 (s, 3H), 3.42 (t, 2H), 2.85 (t, 2H), 2.63 (m, 4H), 2.32 (s, 3H), 2.32 (t, 2H), 2.28 (m, 4H), 2.11 (s, 3H), 1.98 (m, 2H), 1.44-0.88 (m, 12H), 0.84 (s, 6H); ¹³C NMR (125 MHz, dmso-d6) 6 ppm 140.0, 137.7, 130.2, 126.4, 122.3, 122.1, 119.0, 116.9, 114.3, 66.8, 59.5, 59.0, 58.2, 55.6, 55.1, 46.1, 45.4, 30.1, 24.3, 21.7, 12.6, 10.9; HRMS-ESI (m/z): [M+2H]2+ calcd for C₅₂H₆₄N₁₀O₄S: 462.2416. found: 462.2416.

### Preparation 14: 3,6-dichloro-4-(3-iodopropyl)-5-methyl-pyridazine

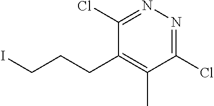

After stirring PPh₃ (59.3 g, 2 eq), imidazole (15.4 g, 2 eq), and iodine (57.4 g, 2 eq) in 560 mL of DCM for 15 min, 25.0 g of Preparation 1 (113 mmol) was added and stirred for 2 h. The product was purified via flash chromatography using heptane and EtOAc as eluents to give 34.7 g of the desired product (92%). ¹H NMR (500 MHz, DMSO-d₆) δ ppm 3.41 (t, 2H), 2.89 (m, 2H), 2.43 (s, 3H), 1.97 (m, 2H); ¹³C NMR (125 MHz, DMSO-d₆) δ ppm 157.7, 156.8, 141.5, 140.2, 31.4, 31.1, 16.7, 7.8; HRMS (ESI) [M]⁺ calcd for C₈H₉Cl₂IN₂: 330.9266. found 330.9255.

### Preparation 15: methyl 5-[3-(methylamino)propyl]-2-[4-methyl-3-[(Z)-[3-(2-trimethylsilyl ethoxymethyl)-1,3-benzothiazol-2-ylidene]amino]-6,7-dihydro-5H-pyrido[2,3-c]pyridazin-8-yl]thiazole-4-carboxylate

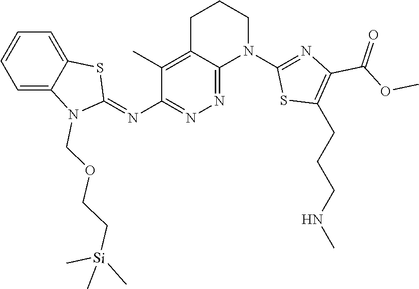 [SMILES: CNCCCc1sc(N2CCCc3c2nnc(/N=c2\sc4ccccc4n2COCC[Si](C)(C)C)c3C)nc1C(=O)OC]

### Step A: methyl 2-(tert-butoxycarbonylamino)-5-[3-[tert-butyl(diphenyl)silyl]oxypropyl]thiazole-4-carboxylate

To 77.0 g of the product of Step C of Preparation 8 (243 mmol), imidazole (33.1 g, 2 eq), and DMAP (1.49 g, 0.05 eq) in 970 mL of DMF was added tert-butyl-chloro-diphenyl-silane (93 mL, 1.5 eq) and stirred for 16 h. The product was purified via flash chromatography using heptane and EtOAc as eluents to give 135 g of the desired product (100%). ¹H NMR (500 MHz, DMSO-d₆) δ ppm 11.63 (s, 1H), 7.60 (d, 4H), 7.45 (t, 2H), 7.42 (t, 4H), 3.74 (s, 3H), 3.67 (t, 2H), 3.20 (t, 2H), 1.87 (qn, 2H), 1.47 (s, 9H), 0.99 (s, 9H); ¹³C NMR (125 MHz, DMSO-d₆) δ ppm 162.8, 156.0, 142.6, 135.6, 135.5, 133.5, 130.3, 128.3, 81.8, 62.9, 51.9, 34.0, 28.3, 27.1, 23.2, 19.2; HRMS (ESI) [M+H]⁺ calcd for C₂₉H₃₉N₂O₅SSi: 555.2349. found 555.2336.

### Step B: methyl 2-[tert-butoxycarbonyl-[3-(3,6-dichloro-5-methyl-pyridazin-4-yl)propyl]amino]-5-[3-[tert-butyl(diphenyl)silyl]oxypropyl]thiazole-4-carboxylate

The product of Step A (35.0 g, 63 mmol), Preparation 14 (25.0 g, 1.2 eq), and Cs₂CO₃ (41.0 g, 2 eq) in 315 mL of acetone were stirred for 1 h. After the reaction mixture was diluted with water and extracted with EtOAc, the combined organic layers were dried, filtered, and concentrated to give the desired product (51.0 g, 106%), which was used in the next step without further purification. ¹H NMR (500 MHz, DMSO-d₆) δ ppm 7.63-7.37 (m, 10H), 4.09 (t, 2H), 3.75 (s, 3H), 3.67 (t, 2H), 3.20 (t, 2H), 2.82 (m, 2H), 2.40 (s, 3H), 1.87 (m, 2H), 1.87 (m, 2H), 1.50 (s, 9H), 0.97 (s, 9H); ¹³C NMR (125 MHz, DMSO-d₆) δ ppm 62.9, 52.0, 46.1, 33.9, 28.1, 27.5, 27.1, 25.9, 23.8, 16.4; HRMS (ESI) [M+H]⁺ calcd for C₃₇H₄₇C₁₂N₄O₅SSi: 757.2413. found 757.2395.

### Step C: methyl 5-[3-[tert-butyl(diphenyl)silyl]oxypropyl]-2-[3-(3,6-dichloro-5-methyl-pyridazin-4-yl)propylamino]thiazole-4-carboxylate

After stirring the product of Step B (51.7 g, 60 mmol) in 1,1,1,3,3,3-hexafluoropropan-2-01 (360 mL) at 100° C. for 18 h, the volatiles were removed under reduced pressure. The crude product was purified via flash chromatography using heptane and EtOAc as eluents to give 36.3 g of the desired product (92%). ¹H NMR (500 MHz, DMSO-d₆) δ ppm 7.71 (t, 1H), 7.63-7.37 (m, 10H), 3.69 (s, 3H), 3.67 (t, 2H), 3.30 (m, 2H), 3.10 (t, 2H), 2.85 (m, 2H), 2.83 (s, 3H), 1.79 (m, 2H), 1.78 (m, 2H), 0.98 (s, 9H); ¹³C NMR (125 MHz, DMSO-d₆) δ ppm 62.9, 51.7, 44.1, 34.2, 28.0, 27.1, 27.0, 23.4, 16.4; HRMS (ESI) [M+H]⁺ calcd for C₃₂H₃Cl₂N₄O₃SSi: 657.1889. found 657.1875.

### Step D: methyl 5-[3-[tert-butyl(diphenyl)silyl]oxypropyl]-2-(3-chloro-4-methyl-6,7-dihydro-5H-pyrido[2,3-c]pyridazin-8-yl)thiazole-4-carboxylate

After mixing the product of Step C (36.0 g, 55 mmol) with Cs₂CO₃ (35.7 g, 2 eq) in 1,4-dioxane (380 mL), the reaction mixture was stirred at 90° C. for 18 h. After diluting the mixture with water, the desired product was collected by filtration (34.0 g, 99%). ¹H NMR (500 MHz, DMSO-d₆) δ ppm 7.61 (d, 4H), 7.43 (t, 2H), 7.42 (t, 4H), 4.26 (t, 2H), 3.77 (s, 3H), 3.70 (t, 2H), 3.23 (t, 2H), 2.90 (t, 2H), 2.33 (s, 3H), 2.04 (qn, 2H), 1.90 (qn, 2H), 1.00 (s, 9H); ¹³C NMR (125 MHz, DMSO-d₆) δ ppm 163.1, 155.3, 151.8, 151.4, 143.2, 136.2, 135.5, 134.7, 133.6, 130.3, 129.0, 128.3, 63.1, 51.9, 46.3, 34.1, 27.1, 24.2, 23.1, 19.8, 19.2, 15.7; HRMS (ESI) [M+H]⁺ calcd for C₃₂H₃₈ClN₄O₃SSi: 621.2122. found 621.2097.

### Step E: methyl 2-[3-(1,3-benzothiazol-2-ylamino)-4-methyl-6,7-dihydro-5H-pyrido[2,3-c]pyridazin-8-yl]-5-[3-[tert-butyl(diphenyl)silyl]oxypropyl]thiazole-4-carboxylate

After mixing the product of Step D (6.21 g, 10 mmol), 1,3-benzothiazol-2-amine (3.0 g, 2 eq), and DIPEA (8.7 mL, 2 eq) in cyclohexanol (50 mL), Pd₂(dba)₃ (915 mg, 0.1 eq) and XantPhos (1.16 g, 0.2 eq) were added, and the reaction mixture was stirred at 140° C. for 1 h. The product was purified via flash chromatography using heptane and EtOAc as eluents to give 5.74 g of the desired product (78%). ¹H NMR (500 MHz, DMSO-d₆) δ ppm 7.77 (br., 1H), 7.70-7.40 (br., 1H), 7.65 (dm, 4H), 7.45-7.38 (m, 6H), 7.36 (brt., 1H), 7.18 (brt., 1H), 4.26 (m, 2H), 3.78 (s, 3H), 3.71 (t, 2H), 3.25 (t, 2H), 2.88 (t, 2H), 2.35 (s, 3H), 2.05 (m, 2H), 1.92 (m, 2H), 1.04 (s, 9H); ¹³C NMR (125 MHz, DMSO-d₆) δ ppm 155.7, 151.5, 129.2, 127.7, 126.5, 122.6, 122.1, 63.0, 51.9, 46.3, 34.2, 27.3, 23.9, 22.9, 20.3, 12.9; HRMS (ESI) [M+H]⁺ calcd for C₃₉H₄₃N₆O₃S₂Si: 735.2607. found 735.2604.

### Step F: methyl 5-[3-[tert-butyl(diphenyl)silyl]oxypropyl]-2-[4-methyl-3-[(Z)-[3-(2-trimethylsilylethoxymethyl)-1,3-benzothiazol-2-ylidene]amino]-6,7-dihydro-5H-pyrido[2,3-c]pyridazin-8-yl]thiazole-4-carboxylate

After cooling the mixture of the product of Step E (1.64 g, 2.2 mmol), DIPEA (0.77 mL, 2 eq), and DMAP (13 mg, 0.05 eq) in DCM (12 mL) to −20° C., 2-(chloromethoxy)ethyl-trimethyl-silane (0.61 mL, 1.55 eq) was added, and the reaction mixture was stirred for 18 h. The product was purified via flash chromatography using DCM and EtOAc as eluents to give 1.56 g of the desired product (80%). LC/MS (C₄₅H₅₇N₆O₄S₂Si₂) 865.4 [M+H]⁺.

### Step G: methyl 5-(3-hydroxypropyl)-2-[4-methyl-3-[(Z)-[3-(2-trimethylsilylethoxymethyl)-1,3-benzothiazol-2-ylidene]amino]-6,7-dihydro-5H-pyrido[2,3-c]pyridazin-8-yl]thiazole-4-carboxylate

The product of Step F (1.56 g, 1.8 mmol) and a 1 M THF solution of TBAF (2.1 mL, 1.2 eq) were stirred in THF (18 mL) for 4 h. The product was purified via flash chromatography using heptane and EtOAc as eluents to give 950 mg of the desired product (83%). ¹H NMR (500 MHz, DMSO-d₆) δ ppm 7.83 (dm, 1H), 7.44 (dm, 1H), 7.42 (m, 1H), 7.23 (m, 1H), 5.84 (s, 2H), 4.57 (brs, 1H), 4.26 (t, 2H), 3.80 (s, 3H), 3.72 (m, 2H), 3.48 (t, 2H), 3.14 (m, 2H), 2.86 (t, 2H), 2.36 (s, 3H), 2.04 (m, 2H), 1.81 (m, 2H), 0.91 (m, 2H), −0.11 (s, 9H); ¹³C NMR (125 MHz, DMSO-d₆) δ ppm 127.1, 123.3, 123.2, 111.9, 72.9, 66.7, 60.6, 51.9, 46.4, 35.0, 23.8, 23.2, 20.4, 17.8, 13.0, −1.0; HRMS (ESI) [M+H]⁺ calcd for C₂₉H₃₉N₆O₄S₂Si: 627.2243. found 627.2236.

### Step H: methyl 5-(3-iodopropyl)-2-[4-methyl-3-[(Z)-[3-(2-trimethylsilylethoxymethyl)-1,3-benzothiazol-2-ylidene]amino]-6,7-dihydro-5H-pyrido[2,3-c]pyridazin-8-yl]thiazole-4-carboxylate

After stirring PPh₃ (594 mg, 1.1 eq), imidazole (154 mg, 1.1 eq), and iodine (574 mg, 1.1 eq) in DCM (10 mL) for 30 min, the product of Step G (1.29 g, 2 mmol) in 1 mL of DCM was added at 0° C. After 18 h stirring at room temperature, the product was purified via flash chromatography using heptane, EtOAc and MeOH (with 0.6 M NH₃) as eluents to give 850 mg of the desired product (56%). ¹H NMR (500 MHz, DMSO-d₆) δ ppm 7.84-7.19 (m, 4H), 5.81 (s, 2H), 4.24 (t, 2H), 3.81 (s, 3H), 3.71 (m, 2H), 3.34 (t, 2H), 3.18 (t, 2H), 2.82 (t, 2H), 2.32 (s, 3H), 2.14 (m, 2H), 2.03 (m, 2H), 0.90 (m, 2H), −0.11 (s, 9H); HRMS (ESI) [M+H]⁺ calcd for C₂₉H₃₈IN₆O₃S₂Si: 737.1261. found 737.1272.

### Step I: methyl 5-[3-(methylamino)propyl]-2-[4-methyl-3-[(Z)-[3-(2-trimethylsilylethoxy methyl)-1,3-benzothiazol-2-ylidene]amino]-6,7-dihydro-5H-pyrido[2,3-c]pyridazin-8-yl]thiazole-4-carboxylate

To the product of Step H (850 mg, 1.1 mmol) in NMP (1.0 mL) and MeCN (11 mL) was added 4.0 mL of a 2 M solution of methanamine in THF (7 eq), and the reaction mixture was stirred at 50° C. for 3 h. The product was purified via flash chromatography using heptane, EtOAc and MeOH (with 0.6 M NH₃) as eluents to give 500 mg of the desired product (67%). ¹H NMR (500 MHz, dmso-d6) δ ppm 7.82 (d, 1H), 7.45 (d, 1H), 7.42 (td, 1H), 7.24 (td, 1H), 5.84 (s, 2H), 4.26 (m, 2H), 3.81 (s, 3H), 3.72 (t, 2H), 3.15 (t, 2H), 2.87 (t, 2H), 2.58 (t, 2H), 2.37 (s, 3H), 2.32 (s, 3H), 2.04 (m, 2H), 1.81 (m, 2H), 0.91 (t, 2H), −0.11 (s, 9H); ¹³C NMR (125 MHz, dmso-d6) δ ppm 127.1, 123.3, 123.1, 111.8, 72.9, 66.7, 51.9, 50.8, 46.4, 36, 31.1, 24.2, 23.8, 20.3, 17.8, 13, −0.9; HRMS (ESI) [M+H]⁺ calcd for C₃₀H₄₂N₇O₃S₂Si: 640.2560. found 640.2560.

### Preparation 16: 6-[5-azidopentyl-[6-(1,3-benzothiazol-2-ylamino)-5-methyl-pyridazin-3-yl]amino]-3-[1-[[3-[2-(dimethylamino)ethoxy]-5,7-dimethyl-1-adamantyl]methyl]-5-methyl-pyrazol-4-yl]pyridine-2-carboxylic acid

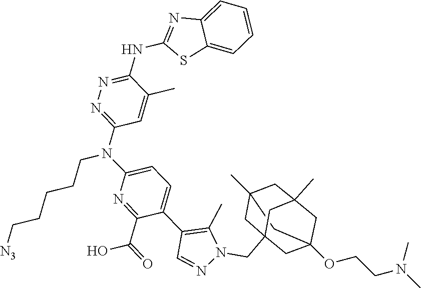 [SMILES: Cc1cc(N(CCCCCN=[N+]=[N-])c2ccc(-c3cnn(CC45CC6(C)CC(C)(C4)CC(OCCN(C)C)(C6)C5)c3C)c(C(=O)O)n2)nnc1Nc1nc2ccccc2s1]

To Preparation 7 (100 mg, 0.092 mmol) in acetonitrile (1.9 mL) was added pyridine, hydrogen fluoride (1:1) (25 eq) and the reaction was stirred at 60° C. for 4 h. The product was purified by preparative reversed phase chromatography to give the desired product (40 mg, 52%). ¹H NMR (500 MHz, dmso-d6) δ ppm 7.83 (dm, 1H), 7.57 (brd, 1H), 7.49 (d, 1H), 7.47 (q, 1H), 7.37 (m, 1H), 7.36 (s, 1H), 7.20 (m, 1H), 7.02 (d, 1H), 4.19 (m, 2H), 3.85 (s, 2H), 3.58 (t, 2H), 3.33 (t, 2H), 2.78 (t, 2H), 2.50 (s, 6H), 2.34 (d, 3H), 2.19 (s, 3H), 1.75 (m, 2H), 1.62 (m, 2H), 1.45-1.04 (m, 12H), 1.44 (m, 2H), 0.89 (s, 6H); ¹³C NMR (125 MHz, dmso-d6) δ ppm 140.7, 138.0, 126.3, 124.2, 122.6, 121.9, 117.6, 112.5, 58.6, 58.0, 57.7, 51.3, 48.2, 44.8, 29.0, 28.5, 27.7, 24.1, 17.1, 11.4; HRMS (ESI) [M+H]⁺ calcd for C₄₄H₅₇N₁₂O₃S: 833.4397. found 833.4395.

### Preparation 17: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]pyridine-2-carboxylate

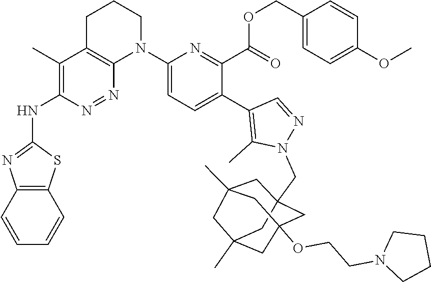 [SMILES: COc1ccc(COC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)cc1]

To the product from Step G of Preparation 4 in a 1:1 mixture of acetonitrile and N-methyl-2-pyrrolidone (10 ml/mmol) was added the pyrrolidine (7 eq) and the reaction mixture was stirred at 60° C. for 18 h. After the purification of the product by preparative reversed phase chromatography, the desired product was obtained. HRMS-ESI (m/z): [M+2H]²⁺ calcd for C₅₂H₆₂N₉O₄S: 454.7356. found 454.7365.

### Preparation 18: 2-[4-Aminobutyl-[6-(1,3-benzothiazol-2-ylamino)-5-methyl-pyridazin-3-yl]amino]-5-[3-[4-[3-(dimethylamino)prop-1-ynyl]-2-fluoro-phenoxy]propyl]thiazole-4-carboxylic acid

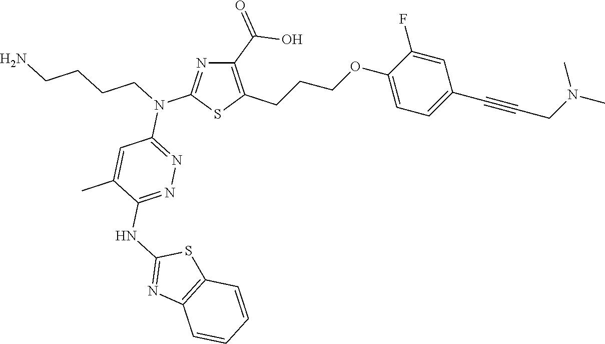 [SMILES: Cc1cc(N(CCCCN)c2nc(C(=O)O)c(CCCOc3ccc(C#CCN(C)C)cc3F)s2)nnc1Nc1nc2ccccc2s1]

### Step A: methyl 2-(tert-butoxycarbonylamino)-5-iodo-thiazole-4-carboxylate

50.00 g of methyl 2-(tert-butoxycarbonylamino)thiazole-4-carboxylate (193.55 mmol, 1 eq.) was suspended in 600 mL dry MeCN. 52.25 g of N-iodo succinimide (232.30 mmol, 1.2 eq.) were added and the resulting mixture was stirred overnight at room temperature. The reaction mixture was diluted with saturated brine, then it was extracted with EtOAc. The combined organic layers were extracted with 1 M Na₂S₂O₃, then with brine again. Then dried over Na₂SO₄, filtered and the filtrate was concentrated under reduced pressure. The crude product was purified via flash column chromatography using heptane as eluent to obtain 60 g (156 mmol, 80%) of the desired product. ¹H NMR (400 MHz, DMSO-d₆) δ ppm 12.03/11.06 (br s), 3.78 (s, 3H), 1.47 (s, 9H); ¹³C NMR (400 MHz, DMSO-d₆) δ ppm 153.8, 82.5, 77.7, 52.3, 28.3; HRMS-ESI (m/z): [M+H]⁺ calcd for C₁₀H₁₄IN₂O₄S: 384.9713. found 384.9708.

### Step B: methyl 2-(tert-butoxycarbonylamino)-5-(3-hydroxyprop-1-ynyl)thiazole-4-carboxylate

A 500 mL oven-dried, one-necked, round-bottom flask was equipped with a PTFE-coated magnetic stirring bar and fitted with a reflux condenser. It was charged with 9.6 g of the product from Step A (25 mmol, 1 eq.), 2.80 g of prop-2-yn-1-ol (2.91 mL, 50 mmol, 2 eq.) and 36.10 g of DIPA (50 mL, 356.8 mmol, 14.27 eq.) then 125 mL of dry THF were added and the system was flushed with argon. After 5 minutes stirring under inert atmosphere 549 mg of Pd(PPh₃)₂Cl₂ (1.25 mmol, 0.05 eq.) and 238 mg of CuI (1.25 mmol, 0.05 eq.) were added. The resulting mixture was then warmed up to 60° C. and stirred at that temperature until no further conversion was observed. Celite was added to the reaction mixture and the volatiles were removed under reduced pressure. Then it was purified via flash column chromatography using heptane and EtOAc as eluents to give 7.30 g (23 mmol, 93%) of the desired product as a yellow solid. ¹H NMR (400 MHz, DMSO-d₆) δ ppm 12.10 (br s, 1H), 5.45 (t, 1H), 4.36 (d, 2H), 3.79 (s, 3H), 1.48 (s, 9H); ¹³C NMR (125 MHz, DMSO-d₆) δ ppm 161.3, 142.4, 118.1, 101.4, 73.9, 52.4, 50.2, 28.3; HRMS-ESI (m/z): [M+H]⁺ calcd for C₁₃H₁₇N₂O₅S: 313.0853. found 313.0866.

### Step C: methyl 2-(tert-butoxycarbonylamino)-5-(3-hydroxypropyl)thiazole-4-carboxylate

An 1 L oven-dried pressure bottle equipped with a PTFE-coated magnetic stirring bar was charged with 44.75 g of the product from Step B (143.3 mmol, 1 eq.), 7.62 g of Pd/C (7.17 mmol, 0.05 eq.) in 340 mL of ethanol, and then placed under a nitrogen atmosphere using hydrogenation system. After that it was filled with 4 bar H₂ gas and stirred at rt overnight. Full conversion was observed, but only the olefin product was formed. After filtration of the catalyst through a pad of Celite the whole procedure was repeated with 5 mol % new catalyst. The resulting mixture was stirred overnight to get full conversion. Celite was added to the reaction mixtures and the volatiles were removed under reduced pressure. Then it was purified via flash column chromatography using heptane and EtOAc as eluents to give 31.9 g (101 mmol, 70%) of the desired product as light-yellow crystals. ¹H NMR (500 MHz, DMSO-d₆) δ ppm 11.61 (br s, 1H), 4.54 (t, 1H), 3.76 (s, 3H), 3.43 (m, 2H), 3.09 (t, 2H), 1.74 (m, 2H), 1.46 (s, 9H); ¹³C NMR (125 MHz, DMSO-d₆) δ ppm 162.8, 143.1, 135.4, 60.3, 51.9, 34.5, 28.3, 23.4; HRMS-ESI (m/z): [M+H]⁺ calcd for C₁₃H₂₁N₂O₅S: 317.1166. found 317.1164.

### Step D: methyl 2-{[(tert-butoxy)carbonyl]amino}-5-[3-(2-fluoro-4-iodophenoxy)propyl]-1,3-thiazole-4-carboxylate

A 250 mL oven-dried, one-necked, round-bottomed flask equipped with a PTFE-coated magnetic stirring bar, was charged with 3.40 g of 2-fluoro-4-iodo-phenol (14 mmol, 1 eq.), 5.00 g of the product from Step C (16 mmol, 1.1 equiv) and 4.10 g of PPh₃ (16 mmol, 1.1 eq.) and 71 mL of dry toluene. After 5 min stirring under nitrogen atmosphere, 3.10 mL of DIAD (3.20 g, 16 mmol, 1.1 eq.) were added in one portion while the reaction mixture warmed up. Then the reaction mixture was heated up to 50° C. and stirred at that temperature for 30 min, when the reaction reached complete conversion. The reaction mixture was directly injected onto a preconditioned silica gel column, and then it was purified via flash column chromatography using heptane and EtOAc as eluents. The crude product was crystallized from MeOH to give 4.64 g (9.24 mmol, 66%) of the desired product. ¹H NMR (500 MHz, DMSO-d₆) δ ppm 11.64 (br s, 1H), 7.59 (dd, 1H), 7.45 (dd, 1H), 6.98 (t, 1H), 4.06 (t, 2H), 3.73 (s, 3H), 3.22 (t, 2H), 2.06 (m, 2H), 1.46 (s, 9H); ¹³C NMR (125 MHz, DMSO-d₆) δ ppm 134.0, 124.9, 117.6, 68.2, 51.9, 30.5, 28.3, 23.2; HRMS-ESI (m/z): [M+H]⁺ calcd for C₁₉H₂₃N₂O₅FSI: 537.0351. found 537.0348.

### Step E: methyl 2-(tert-butoxycarbonylamino)-5-[3-[4-[3-(dimethylamino)prop-1-ynyl]-2-fluoro-phenoxy]propyl]thiazole-4-carboxylate

A 250 mL oven-dried, one-necked, round-bottom flask was equipped with a PTFE-coated magnetic stirring bar and fitted with a reflux condenser. It was charged with 5.36 g of Step D (10 mmol, 1 eq.), 1.66 g of N,N-dimethylprop-2-yn-1-amine (20 mmol, 2 eq.) and 20 mL of DIPA (142.7 mmol, 14.27 eq.) then 50 mL of dry THF were added and the system was flushed with argon. After 5 minutes stirring under inert atmosphere 220 mg of Pd(PPh₃)₂Cl₂ (0.5 mmol, 0.05 eq.) and 95 mg of CuI (0.5 mmol, 0.05 eq.) were added. The resulting mixture was then warmed up to 60° C. and stirred at that temperature until no further conversion was observed. Celite was added to the reaction mixture and the volatiles were removed under reduced pressure. Then it was purified via flash column chromatography using DCM and MeOH (1.2% NH₃) as eluents to give 4.5 g (7.8 mmol, 78%) of the desired product. ¹H NMR (500 MHz, DMSO-d₆) δ ppm 11.66 (s, 1H), 7.29 (dd, 1H), 7.19 (m, 1H), 7.12 (t, 1H), 4.09 (t, 2H), 3.73 (s, 3H), 3.44 (s, 2H), 3.23 (t, 2H), 2.24 (s, 6H), 2.07 (m, 2H), 1.45 (s, 9H); ¹³C NMR (125 MHz, DMSO-d₆) δ ppm 162.8, 147.3, 129.0, 119.2, 115.4, 84.3, 68.0, 51.9, 48.1, 44.2, 30.6, 28.3, 23.2; HRMS-ESI (m/z): [M+H]⁺ calcd for C₂₄H₃₁FN₃O₅S: 492.1963. found 492.1956.

### Step F: methyl 2-[tert-butoxycarbonyl-[4-(tert-butoxycarbonylamino)butyl]amino]-5-[3-[4-[3-(dimethylamino)prop-1-ynyl]-2-fluoro-phenoxy]propyl]thiazole-4-carboxylate

Using Mitsunobu General Procedure starting from 250 mg of Step E (0.51 mmol, 1 eq.) and 193 mg of tert-butyl N-(4-hydroxybutyl)carbamate (1.02 mmol, 2 eq.) as the appropriate alcohol, 220 mg (65%) of the desired product were obtained. ¹H NMR (400 MHz, DMSO-d₆) δ ppm 7.30 (dd, 1H), 7.21 (d, 1H), 7.13 (t, 1H), 6.80 (t, 1H), 4.10 (t, 2H), 4.01-3.95 (m, 2H), 3.75 (s, 3H), 3.45 (s, 2H), 3.22 (t, 2H), 2.91 (q, 2H), 2.25 (s, 6H), 2.08 (qv, 2H), 1.63-1.54 (m, 2H), 1.50 (s, 9H), 1.40-1.35 (m, 2H), 1.35 (s, 9H); LC-MS-ESI (m/z): [M+H]⁺ calcd for C₃₃H₄₈FN₄O₇S: 663.3. found 663.4.

### Step G: methyl 2-[4-(tert-butoxycarbonylamino)butylamino]-5-[3-[4-[3-(dimethylamino)prop-1-ynyl]-2-fluoro-phenoxy]propyl]thiazole-4-carboxylate

Using Deprotection with HFIP General Procedure starting from 215 mg of the product from Step F (0.33 mmol, 1 eq.) as the appropriate Boc protected amine, 137 mg (75%) of the desired product were obtained. ¹H NMR (400 MHz, DMSO-d₆) δ ppm 7.57 (t, 1H), 7.30 (d, 1H), 7.21 (d, 1H), 7.12 (t, 1H), 6.81 (t, 1H), 4.07 (t, 2H), 3.69 (s, 3H), 3.42 (s, 2H), 3.17-3.09 (m, 4H), 2.94-2.88 (m, 2H), 2.23 (s, 6H), 2.04-2.00 (m, 2H), 1.53-1.37 (m, 4H), 1.36 (s, 9H); LC-MS-ESI (m/z): [M+H]⁺ calcd for C₂₈H₄₀FN₄O₅S: 563.3. found 563.2.

### Step H: methyl 2-[4-(tert-butoxycarbonylamino)butyl-[5-methyl-6-[(Z)-[3-(2-trimethylsilylethoxymethyl)-1,3-benzothiazol-2-ylidene]amino]pyridazin-3-yl]amino]-5-[3-[4-[3-(dimethylamino)prop-1-ynyl]-2-fluoro-phenoxy]propyl]thiazole-4-carboxylate

Using Buchwald General Procedure II starting from 133 mg of the product from Step G (0.24 mmol, 1 eq.) and 120 mg of Preparation 6 (0.29 mmol, 1.25 eq.) as the appropriate halide, 220 mg (98%) of the desired product were obtained. ¹H NMR (500 MHz, DMSO-d₆) δ ppm 7.84 (d, 1H), 7.69 (s, 1H), 7.47 (d, 1H), 7.44 (td, 1H), 7.31 (dd, 1H), 7.25 (td, 1H), 7.21 (dm, 1H), 7.16 (t, 1H), 6.82 (t, 1H), 5.86 (s, 2H), 4.36 (t, 2H), 4.15 (t, 2H), 3.78 (s, 3H), 3.72 (t, 2H), 3.38 (s, 2H), 3.27 (t, 2H), 2.98 (q, 2H), 2.46 (s, 3H), 2.19 (s, 6H), 2.13 (m, 2H), 1.67 (m, 2H), 1.46 (m, 2H), 1.34 (s, 9H), 0.92 (t, 2H), −0.11 (s, 9H); ¹³C NMR (125 MHz, DMSO-d₆) δ ppm 156.1, 128.9, 127.2, 123.5, 123.2, 119.2, 117.6, 115.5, 112.0, 72.9, 68.4, 66.7, 52.0, 48.1, 46.7, 44.2, 39.8, 31.0, 28.7, 27.2, 24.7, 23.1, 17.9, 17.8, −1.0; HRMS-ESI (m/z): [M+H]⁺ calcd for C₄₆H₆₂FN₈O₆S₂Si: 933.3982. found 933.3995.

### Step I: 2-[4-aminobutyl-[6-(1,3-benzothiazol-2-ylamino)-5-methyl-pyridazin-3-yl]amino]-5-[3-[4-[3-(dimethylamino)prop-1-ynyl]-2-fluoro-phenoxy]propyl]thiazole-4-carboxylic acid

Using Deprotection and Hydrolysis General Procedure followed by repurification via reverse phase preparative chromatography (C18, 0.1% TFA in water:MeCN) starting from the product from Step H, the TFA-salt of the desired product was obtained. ¹H NMR (400/500 MHz, dmso-d6) δ ppm 7.86 (dm, 1H), 7.66 (brs, 1H), 7.64 (brs, 3H), 7.54 (brd, 1H), 7.40 (dd, 1H), 7.39 (m, 1H), 7.31 (dm, 1H), 7.22 (m, 1H), 7.22 (t, 1H), 4.41 (t, 2H), 4.23 (s, 2H), 4.21 (t, 2H), 3.29 (m, 2H), 2.92 (brm, 2H), 2.84 (s, 6H), 2.48 (d, 3H), 2.16 (m, 2H), 1.81 (m, 2H), 1.67 (m, 2H); ¹³C NMR (125 MHz, dmso-d6) δ ppm 129.4, 126.5, 122.6, 122.1, 119.6, 118.7, 116.1, 69.1, 47.3, 46.6, 42.4, 39.1, 31.0, 24.5, 24.4, 23.2, 17.6; HRMS-ESI (m/z): [M+2H]²⁺ calcd for C₃₄H₃₉FN₈O₃S₂: 345.1280. found 345.1281.

### Preparation 19: (4-methoxyphenyl)methyl 6-[[6-(1,3-benzothiazol-2-ylamino)-5-methyl-pyridazin-3-yl]-[4-(methylamino)butyl]amino]-3-[1-[[3-[2-(dimethylamino)ethoxy]-5,7-dimethyl-1-adamantyl]methyl]-5-methyl-pyrazol-4-yl]pyridine-2-carboxylate

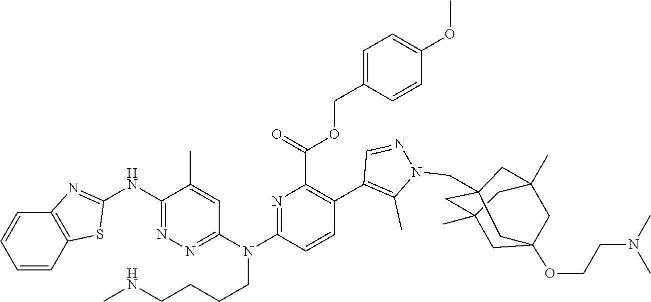 [SMILES: CNCCCCN(c1cc(C)c(Nc2nc3ccccc3s2)nn1)c1ccc(-c2cnn(CC34CC5(C)CC(C)(C3)CC(OCCN(C)C)(C5)C4)c2C)c(C(=O)OCc2ccc(OC)cc2)n1]

### Step A: methyl 3-bromo-6-[tert-butoxycarbonyl-[4-[tert-butoxycarbonyl(methyl) amino]butyl]amino]pyridine-2-carboxylate

To the mixture of 18.4 g (6 eq) of tert-butyl N-(4-hydroxybutyl)-N-methyl-carbamate, 5 g (15.1 mmol) of Preparation 2, Step B, and 24 g (6 eq) of triphenylphosphane in 75 mL of toluene were added dropwise 20.4 mL of (6 eq) tert-butyl N-(4-hydroxybutyl)-N-methyl-carbamate and the reaction mixture was stirred at 50° C. for 1 h and purified by column chromatography to give the desired product (100+%). ¹H NMR (400 MHz, dmso-d6) δ ppm 8.13 (d, 1H), 7.72 (d, 1H), 3.89 (s, 3H), 3.82 (t, 2H), 3.13 (t, 2H), 2.71 (s, 3H), 1.49-1.30 (br., 18H), 1.49 (br., 2H), 1.41 (br., 2H); ¹³C NMR (100 MHz, dmso-d6) δ ppm 165.4, 155.2, 153.4, 153.1, 147.4, 143.0, 123.1, 111.8, 53.3, 48.1, 46.4, 34.1, 25.9, 25.3; HRMS-ESI (m/z): [M+H]⁺ calcd for C₂₂H₃₅BrN₃O₆: 516.1704. found 516.1705.

### Step B: methyl 6-[tert-butoxycarbonyl-[4-[tert-butoxycarbonyl(methyl)amino]butyl]amino]-3-[1-[[3-[2-[tert-butyl(diphenyl)silyl]oxyethoxy]-5,7-dimethyl-1-adamantyl]methyl]-5-methyl-pyrazol-4-yl]pyridine-2-carboxylate

The mixture of 7.7 g (14.9 mmol) of the product of Step A and 10.2 g (1 eq) of Preparation 3 in a mixture of 90 mL of 1,4-dioxane and 15 mL of water was treated with 14.6 (3 eq) of Cs₂CO₃ and 0.66 g (0.1 eq) of Pd(AtaPhos)₂Cl₂. Then, the reaction was stirred at 80° C. for 0.5 h. The product was purified by column chromatography using heptane and EtOAc as eluents on silica gel to give 10.44 g (70%) of the desired product. 1H NMR (400 MHz, dmso-d6) δ ppm 7.75 (d, 1H), 7.72 (d, 1H), 7.69-7.35 (m, 1 OH), 7.37 (s, 1H), 3.86 (m, 2H), 3.86 (s, 2H), 3.67 (t, 2H), 3.65 (s, 3H), 3.46 (t, 2H), 3.13 (t, 2H), 2.71 (s, 3H), 2.11 (s, 3H), 1.52 (br., 2H), 1.47/1.33 (s+brs., 18H), 1.43 (br., 2H), 1.4-0.95 (m, 12H), 0.97 (s, 9H), 0.84 (s, 6H); ¹³C NMR (100 MHz, dmso-d6) δ ppm 167.1, 155.2, 153.7, 152.3, 147.2, 140.7, 137.4, 121.5, 115.2, 64.4, 61.7, 59.0, 52.6, 48.1, 46.4, 34.1, 30.1, 28.5/28.3, 27.1, 26.0, 25.1, 10.8; HRMS-ESI (m/z): [M+H]⁺ calcd for C₂₂H₃₅BrN₃O₆: 992.5927. found 992.5922.

### Step C: methyl 6-[tert-butoxycarbonyl-[4-[tert-butoxycarbonyl(methyl)amino]butyl]amino]-3-[1-[[3-(2-hydroxyethoxy)-5,7-dimethyl-1-adamantyl]methyl]-5-methyl-pyrazol-4-yl]pyridine-2-carboxylate

The mixture of 5 g (5.04 mmol) of the product of Step B and 6.05 mL (1.2 eq) of 1 M solution of TBAF in THF in 50 mL of THF was stirred for 0.5 h. Then, the reaction was quenched with NH₄Cl solution and extracted with EtOAc. The combined organic layers were concentrated and purified by column chromatography using heptane and EtOAc as eluents on silica gel to give 3.63 g (95%) of the desired product. ¹H NMR (400 MHz, DMSO-d₆) δ ppm 7.78 (d, 1H), 7.74 (d, 1H), 7.38 (s, 1H), 4.45 (t, 1H), 3.87 (s, 2H), 3.86 (t, 2H), 3.69 (s, 3H), 3.40 (q, 2H), 3.34 (t, 2H), 3.14 (t, 2H), 2.71 (s, 3H), 2.12 (s, 3H), 1.52 (brm, 2H), 1.47 (s, 9H), 1.45 (brm, 2H), 1.38 (s, 2H), 1.34 (s, 9H), 1.30/1.24 (d+d, 4H), 1.17/1.11 (d+d, 4H), 1.07/0.99 (d+d, 2H), 0.86 (s, 6H); ¹³C NMR (100 MHz, DMSO-d₆) δ ppm 140.8, 137.5, 121.6, 62.1, 61.5, 58.9, 52.7, 50.1, 48.1, 47.0, 46.4, 46.0, 43.3, 34.1, 30.1, 28.5, 28.3, 25.9, 25.3, 10.8; HRMS-ESI (m/z): [M+H]⁺ calculated for C₄₁H₆₄N₅O₈: 754.4749. found 754.4751.

### Step D: methyl 6-[tert-butoxycarbonyl-[4-[tert-butoxycarbonyl(methyl)amino]butyl]amino]-3-[1-[[3,5-dimethyl-7-[2-(p-tolylsulfonyloxy)ethoxy]-1-adamantyl]methyl]-5-methyl-pyrazol-4-yl]pyridine-2-carboxylate

The mixture of 3.6 g (4.78 mmol) of the product of Step C and 2 mL (3 eq) of triethylamine in 62 mL of DCM was treated with 2.34 g (1.5 eq) of p-tolylsulfonyl 4-methylbenzenesulfonate at 0° C. Then, the reaction was stirred at room temperature for 0.5 h, diluted with saturated NaHCO₃ solution, and extracted with EtOAc. The combined organic layers were concentrated and purified by column chromatography using heptane and EtOAc as eluents on silica gel to give 3.82 g (88%) of the desired product. ¹H NMR (400 MHz, DMSO-d₆) δ ppm 7.79 (d, 1H), 7.77 (d, 2H), 7.75 (d, 1H), 7.46 (d, 2H), 7.39 (s, 1H), 4.06 (t, 2H), 3.87 (t, 2H), 3.85 (s, 2H), 3.68 (s, 3H), 3.49 (t, 2H), 3.15 (t, 2H), 2.72 (s, 3H), 2.41 (s, 3H), 2.12 (s, 3H), 1.53 (brm, 2H), 1.48 (s, 9H), 1.44 (brm, 2H), 1.35 (s, 9H), 1.28 (s, 2H), 1.17/1.09 (d+d, 4H), 1.13/1.1 (d+d, 4H), 1.03/0.96 (d+d, 2H), 0.84 (, 6H); ¹³C NMR (100 MHz, DMSO-d₆) δ ppm 140.8, 137.5, 130.6, 128.1, 121.6, 71.5, 58.8, 58.4, 52.8, 49.9, 48.1, 46.6, 46.5, 45.9, 42.9, 34.1, 30.1, 28.5, 28.2, 26.0, 25.2, 21.6, 10.9; HRMS-ESI (m/z): [M+H]⁺ calculated for C₄₈H₇₀N₅O₁₀S: 908.4838. found 908.4842.

### Step E: methyl 6-[tert-butoxycarbonyl-[4-[tert-butoxycarbonyl(methyl)amino]butyl]amino]-3-[1-[[3-[2-(dimethylamino)ethoxy]-5,7-dimethyl-1-adamantyl]methyl]-5-methyl-pyrazol-4-yl]pyridine-2-carboxylate

After the treatment of 3.7 g (4.07 mmol) of the product of Step D with 20.37 mL (10 eq) of a 2 M solution of dimethylamine in MeOH at 50° C. for 2 h, the mixture was diluted with 10% aqueous K₂CO₃ solution and extracted with DCM. The combined organic layers were dried and concentrated to give 3.17 g (99%) of the desired product. ¹H NMR (400 MHz, DMSO-d₆) δ ppm 7.78 (d, 1H), 7.75 (d, 1H), 7.38 (s, 1H), 3.87 (s, 2H), 3.87 (t, 2H), 3.72 (t, 2H), 3.70 (s, 3H), 3.15 (t, 2H), 2.72 (s, 3H), 2.35 (t, 2H), 2.16 (s, 6H), 2.13 (s, 3H), 1.52 (brm, 2H), 1.48 (s, 9H), 1.44 (brm, 2H), 1.38 (s, 2H), 1.35 (s, 9H), 1.31/1.25 (d+d, 4H), 1.18/1.12 (d+d, 4H), 1.08/1.00 (d+d, 2H), 0.87 (s, 6H); ¹³C NMR (100 MHz, DMSO-d₆) δ ppm 140.8, 137.5, 121.6, 59.7, 58.9, 58.4, 52.8, 50.0, 48.2, 46.9, 46.4, 46.0, 46.0, 43.2, 34.1, 30.2, 28.5, 28.3, 26.0, 25.2, 10.8; HRMS-ESI (m/z): [M+H]⁺ calculated for C₄₃H₆₉N₆O₇: 781.5222. found 781.5219.

### Step F: methyl 6-[4-[tert-butoxycarbonyl(methyl)amino]butylamino]-3-[1-[[3-[2-(dimethylamino)ethoxy]-5,7-dimethyl-1-adamantyl]methyl]-5-methyl-pyrazol-4-yl]pyridine-2-carboxylate

The product from Step E (3.17 g, 4.06 mmol) in 1,1,1,3,3,3-hexafluoroisopropanol (24 mL) was stirred at 110° C. for 18 h. Purification by column chromatography (silica gel, heptane and EtOAc as eluents) afforded the desired product (1.03 g, 37%). ¹H NMR (400 MHz, DMSO-d₆) δ ppm 7.31 (d, 1H), 7.22 (s, 1H), 6.81 (t, 1H), 6.61 (d, 1H), 3.82 (s, 2H), 3.60 (s, 3H), 3.46 (t, 2H), 3.23 (q, 2H), 3.17 (t, 2H), 2.75 (brs, 3H), 2.53 (t, 2H), 2.28 (s, 6H), 2.06 (s, 3H), 1.52 (qn, 2H), 1.48 (qn, 2H), 1.37 (s, 2H), 1.37 (s, 9H), 1.31/1.25 (d+d, 4H), 1.15/1.1 (d+d, 4H), 1.07/0.99 (d+d, 2H), 0.86 (s, 6H); ¹³C NMR (100 MHz, DMSO-d₆) δ ppm 140.1, 137.5, 110.0, 59.0, 58.9, 57.7, 52.2, 50.0, 48.0, 46.8, 46.0, 45.4, 43.2, 40.9, 34.1, 30.2, 28.6, 26.5, 25.3, 10.8; HRMS-ESI (m/z): [M+H]⁺ calculated for C₃₈H₆₁N₆O₅: 681.4698. found 681.4702.

### Step G: methyl 6-[4-[tert-butoxycarbonyl(methyl)amino]butyl-[5-methyl-6-[(Z)-[3-(2-trimethylsilylethoxymethyl)-1,3-benzothiazol-2-ylidene]amino]pyridazin-3-yl]amino]-3-[1-[[3-[2-(dimethylamino)ethoxy]-5,7-dimethyl-1-adamantyl]methyl]-5-methyl-pyrazol-4-yl]pyridine-2-carboxylate

The mixture of the product of Step F (700 mg, 1.03 mmol), the product of Preparation 6 (711.3 mg, 1.7 eq), diisopropylethylamine (0.54 mL, 3 eq), Cs₂CO₃ (1.0 g, 3 eq), Pd₂(dba)₃ (94 mg, 0.1 eq), and XantPhos (119 mg, 0.2 eq) in 1,4-dioxane (5 mL) was stirred at 120° C. for 1 h. After quenching with brine and extracting with EtOAc, the organic phases were dried, concentrated and purified by column chromatography to give the desired product (1.17 g). ¹H NMR (500 MHz, dmso-d6) δ ppm 7.79 (d, 1H), 7.61 (d, 1H), 7.55 (s, 1H), 7.47 (d, 1H), 7.43 (t, 1H), 7.35 (s, 1H), 7.25 (t, 1H), 7.19 (d, 1H), 5.87 (s, 2H), 4.21 (br, 2H), 3.86 (s, 2H), 3.72 (t, 2H), 3.67 (s, 3H), 3.43 (t, 2H), 3.18 (t, 2H), 2.72 (s, 3H), 2.39 (t, 2H), 2.36 (s, 3H), 2.18 (s, 6H), 2.13 (s, 3H), 1.63 (qn, 2H), 1.52 (qn, 2H), 1.37 (s, 2H), 1.30/1.24 (d+d, 4H), 1.30 (s, 9H), 1.16/1.11 (d+d, 4H), 1.07/0.99 (d+d, 2H), 0.92 (t, 2H), 0.86 (s, 6H), −0.10 (s, 9H); ¹³C NMR (125 MHz, dmso-d6) δ ppm 141.2, 137.5, 127.2, 124.1, 123.5, 123.1, 114.6, 112, 72.9, 66.7, 59.6, 58.9, 58.3, 52.6, 50.1, 48.1, 48.0, 46.9, 46.0, 45.9, 43.3, 34.2, 30.2, 28.5, 25.3, 25.3, 17.9, 17.3, 10.8, −0.9; HRMS-ESI (m/z): [M+2H]²⁺ calculated for C₅₆H₈₄N₁₀O₆SSi: 526.3027. found 526.3026.

### Step H: 6-[[6-(1,3-benzothiazol-2-ylamino)-5-methyl-pyridazin-3-yl]-[4-(methylamino)butyl] amino]-3-[1-[[3-[2-(dimethylamino)ethoxy]-5,7-dimethyl-1-adamantyl]methyl]-5-methyl-pyrazol-4-yl]pyridine-2-carboxylic acid

The product of Step G (1100 mg, 0.84 mmol) in 1,4-dioxane (4 mL) was treated with a 1 M solution of hydrogen chloride in 1,4 dioxane (40 eq) for 1 h. After concentration of the reaction, the residue was treated with cc NaHCO₃ solution and extracted with DCM. The organic phases were concentrated and purified by column chromatography using DCM and MeOH (1.2% NH₃) as eluents on silica gel to give the desired product (86%). ¹H NMR (400 MHz, DMSO-d₆) δ ppm 7.85 (d, 1H), 7.61 (d, 1H), 7.55 (d, 1H), 7.53 (s, 1H), 7.37 (t, 1H), 7.34 (s, 1H), 7.19 (t, 1H), 7.16 (t, 1H), 4.16 (t, 2H), 4.09 (br., 2H), 3.86 (s, 2H), 3.67 (s, 3H), 3.41 (t, 2H), 2.51 (m, 2H), 2.36 (s, 3H), 2.3 (t, 2H), 2.26 (s, 3H), 2.13 (s, 3H), 2.12 (s, 6H), 1.7 (m, 2H), 1.47 (m, 2H), 1.41-0.94 (m, 12H), 0.86 (s, 6H); ¹³C NMR (100 MHz, DMSO-d₆) δ ppm 141.2, 137.5, 126.4, 125.0, 122.4, 122.0, 117.5, 114.2, 59.9, 58.9, 58.6, 52.6, 51.5, 48.3, 46.2, 36.3, 30.2, 26.7, 25.9, 17.3, 10.9; HRMS-ESI [M+H]⁺ calculated for C₄₅H₆₁N₁₀O₃S: 821.4643. found 821.4641.

### Step I: (4-methoxyphenyl)methyl 6-[[6-[(Z)-3H-1,3-benzothiazol-2-ylideneamino]-5-methyl-pyridazin-3-yl]-[4-(methylamino)butyl]amino]-3-[1-[[3-[2-(dimethylamino)ethoxy]-5,7-dimethyl-1-adamantyl]methyl]-5-methyl-pyrazol-4-yl]pyridine-2-carboxylate

The product of Step H (590 mg, 0.72 mmol) and (4-methoxyphenyl)methanol (267 uL, 3.0 eq) were suspended in dry toluene (15 mL), and then tetraethoxytitanium (30 uL, 0.2 eq) was added. The reaction mixture was then refluxed for 2 h. The product was purified by column chromatography using DCM and MeOH (1.2% NH₃) as eluents to give the desired product (82%). ¹H NMR (500 MHz, DMSO-d₆) δ ppm 7.85 (d, 1H), 7.61 (d, 1H), 7.55 (d, 1H), 7.52 (s, 1H), 7.37 (t, 1H), 7.37 (s, 1H), 7.19 (t, 1H), 7.17 (dm, 2H), 7.16 (t, 1H), 6.88 (dm, 2H), 5.09 (s, 2H), 4.16 (t, 2H), 3.86 (s, 2H), 3.72 (s, 3H), 3.41 (t, 2H), 2.51 (m, 2H), 2.31 (s, 3H), 2.30 (t, 2H), 2.26 (s, 3H), 2.11 (s, 6H), 2.09 (s, 3H), 1.70 (m, 2H), 1.47 (m, 2H), 1.41-0.94 (m, 12H), 0.86 (s, 6H); ¹³C NMR (125 MHz, DMSO-d₆) δ ppm 141.2, 137.6, 130.0, 126.4, 125.1, 122.4, 122.0, 117.5, 114.2, 114.2, 66.6, 59.9, 58.9, 58.6, 55.5, 51.5, 48.3, 46.2, 36.3, 30.2, 26.7, 25.9, 17.3, 10.9; HRMS-ESI [M+H]⁺ calculated for C₅₂H₆₇N₁₀O₄S: 927.5062. found 927.5054 (M+H).

### Preparation 20: (2S,4R)-1-[(2S)-3,3-dimethyl-2-[14-(methylamino)tetradecanoylamino]butanoyl]-4-hydroxy-N-[(1S)-1-[4-(4-methylthiazol-5-yl)phenyl]ethyl]pyrrolidine-2-carboxamide

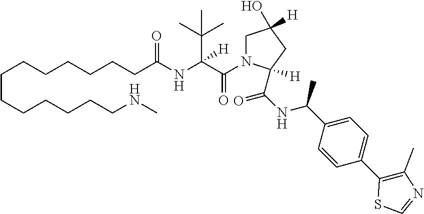 [SMILES: CNCCCCCCCCCCCCCC(=O)N[C@H](C(=O)N1C[C@H](O)C[C@H]1C(=O)N[C@@H](C)c1ccc(-c2scnc2C)cc1)C(C)(C)C]

The mixture of the product of Example 4, Step B (165 mg, 0.20 mmol) and methanamine (1.0 mmol) in acetonitrile (5 mL) was stirred at 70° C. for 18 h and purified by column chromatography using DCM and MeOH as eluents on silica gel to give the desired product (62%). ¹H NMR (500 MHz, dmso-d6) δ ppm 8.98 (s, 1H), 8.38 (d, 1H), 7.79 (d, 1H), 7.43 (d, 2H), 7.38 (d, 2H), 5.10 (brs, 1H), 4.91 (qn, 1H), 4.51 (d, 1H), 4.41 (t, 1H), 4.28 (brm, 1H), 3.61/3.58 (dd+dd, 2H), 2.45 (s, 3H), 2.40 (t, 2H), 2.24/2.09 (m+m, 2H), 2.24 (s, 3H), 2.00/1.79 (m+m, 2H), 1.49-1.44 (m+m, 2H), 1.37 (d, 3H), 1.36 (qn, 2H), 1.27-1.19 (m, 18H), 0.93 (s, 9H); ¹³C NMR (125 MHz, dmso-d6) δ ppm 151.8, 129.3, 126.8, 69.2, 58.9, 56.7, 56.7, 52.0, 48.1, 38.2, 36.7, 35.3, 29.7, 26.9, 25.9, 22.9, 16.4; HRMS-ESI (m/z): [M+H]⁺ calcd for C₃₈H₆₂N₅O₄S: 684.4517. found 684.4525.

### Preparation 21: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]pyridine-2-carboxylic acid

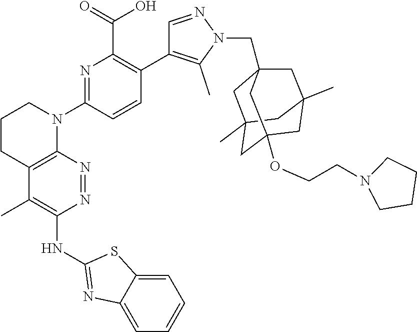 [SMILES: Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(C(=O)O)n1]

To the product of Preparation 4, Step G (500 mg) in acetonitrile (5 mL) was added pyrrolidine (6.5 eq) and the reaction mixture was stirred at 50° C. for 18 h. After treatment of the reaction with KOH (3.6 eq), the mixture was stirred at 50° C. for 2 h. The product was purified by preparative HPLC (using acetonitrile and 5 mM aqueous NH₄HCO₃ solution as eluents) to give the desired product. HRMS-ESI (m/z): [M+H]⁺ calcd for C₄₄H₅₄N₉O₃S: 788.4064. found: 788.4068.

### Preparation 22: N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]piperidine-4-carboxamide

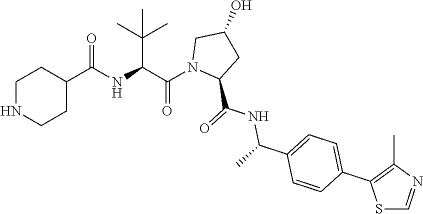 [SMILES: Cc1ncsc1-c1ccc([C@H](C)NC(=O)[C@@H]2C[C@@H](O)CN2C(=O)[C@@H](NC(=O)C2CCNCC2)C(C)(C)C)cc1]

### Step A: tert-butyl 4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]piperidine-1-carboxylate

Using of General procedure for the acylation of VHL ligands starting from (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide; hydrochloride (1:1) (1.04 mmol) and 1-tert-butoxycarbonylpiperidine-4-carboxylic acid as the appropriate acid, 678 mg of the desired product were obtained. ¹H NMR (500 MHz, DMSO-d₆): δ ppm 8.98 (s, 1H), 8.39 (d, 1H), 7.83 (d, 1H), 7.43 (d, 2H), 7.38 (d, 2H), 5.11 (d, 1H), 4.91 (qn, 1H), 4.48 (d, 1H), 4.42 (t, 1H), 4.28 (brm, 1H), 3.93/2.69 (brd+br, 4H), 3.61/3.56 (dd+d, 2H), 2.54 (m, 1H), 2.45 (s, 3H), 2.01/1.78 (m+m, 2H), 1.68/1.56/1.39/1.35 (d+dd/d+dd, 4H), 1.39 (s, 9H), 1.37 (d, 3H), 0.93 (s, 9H); ¹³C NMR (125 MHz, DMSO-d₆) δ ppm 152.0, 129.3, 126.9, 69.3, 59.1, 56.8, 56.7, 48.2, 43.4, 41.4, 38.2, 29.5/28.3, 28.6, 26.9, 23.0, 16.5; HRMS-ESI (m/z): [M+H]⁺ calcd for C₃₄H₅₀N₅O₆S: 656.3476. found: 656.3479.

### Step B: N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]piperidine-4-carboxamide

The mixture of the product of Step A (0.099 mmol) and a 4 M hydrogen chloride solution in 1,4-dioxane (10 eq) in 5 mL of 1,4-dioxane was stirred for 18 h. The volatiles were removed under reduced pressure and it gave 67 mg of the desired product. HRMS-ESI (m/z): [M+H]⁺ calcd for C₂₉H₄₂N₅O₄S: 556.2952. found: 556.2952.

### B. Synthesis and Characterization of Degrader Compounds and Precursors of Bifunctional Bcl-xL Degrader Compounds

Exemplary degrader compounds (DSMs), and precursors of bifunctional degrader compounds, were synthesized using exemplary methods described in this example.

### General Procedure for the Acylation of VHL Ligands

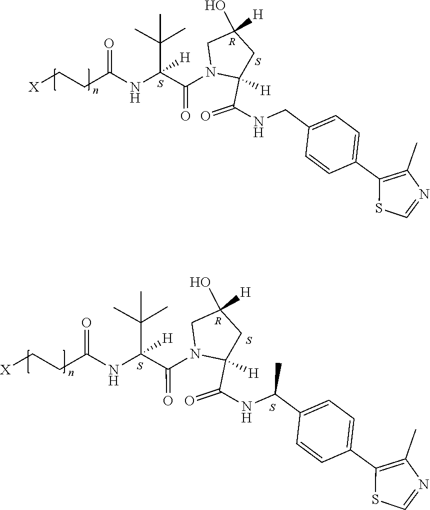

wherein X represents a hydroxyl group or a bromine atom.

To the mixture of the appropriate carboxylic-acid (1 eq), triethylamine (5 eq) and HATU (1.1 eq) in DCM (5 mL/mmol) was added the (2S,4R)-1-[(2S)-2-amino-3,3-dimethyl-butanoyl]-4-hydroxy-N-[(1 S)-1-[4-(4-methylthiazol-5-yl)phenyl]ethyl]pyrrolidine-2-carboxamide, hydrogen chloride (1:1) or the (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (1 eq), and the mixture was stirred until appropriate conversion was achieved. The product was purified by column chromatography (silica gel, heptane, EtOAc, and MeOH as eluents) to give the desired product.

### General Procedure for the Acylation and Deprotection of VHL Ligands

To the mixture of the appropriate protected carboxylic-acid (1.3 eq), triethylamine (5 eq) and HATU (1.1 eq) in DCM (5 mL/mmol) was added the (2S,4R)-1-[(2S)-2-amino-3,3-dimethyl-butanoyl]-4-hydroxy-N-[(1 S)-1-[4-(4-methylthiazol-5-yl)phenyl]ethyl]pyrrolidine-2-carboxamide, hydrogen chloride (1:1) or the (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (1 eq) and the mixture was stirred for 30 min. After concentration, the residue was dissolved in DCM (5 mL/mmol) and TFA (10 mL/mmol), and stirred for 30 min. Product was purified by preparative HPLC (Interchim Method) (C18, using acetonitrile and 0.1% aqueous TFA solution as eluents) to give the desired product.

### General Procedure for the Tosylation of the Hydroxyalkyl VHL Ligand-Derivatives

To the hydroxyalkyl VHL ligand-derivative in DCM (10 mL/mmol) was added TEA (4 eq) and p-tolylsulfonyl 4-methylbenzenesulfonate (2 eq), and the mixture was stirred at RT for 1 h. After quenching the reaction with saturated aqeuous NaHCO₃ solution, and extraction with DCM, the product was purified by column chromatography (silica gel, heptane and EtOAc as eluents) to give the desired product.

### General Procedure for the Nucleophilic Substitution of Fluoro-Thalidomide

The mixture of 2-(2,6-dioxo-3-piperidyl)-4-fluoro-isoindoline-1,3-dione (1 eq), DIPEA (2 eq), and the appropriate amine (2 eq) in 1-methyl-2-pyrrolidinone (5 mL/mmol) was stirred at 90° C. until appropriate conversion was achieved. The crude product was purified by preparative HPLC (Interchim Method) (C18, using acetonitrile and 0.1% aqueous TFA solution as eluents) to give the desired product.

### General Procedure for the Iodination of Hydroxyalkyl Derivative of Thalidomide

To iodine (2 eq), PPh₃ (2 eq), and imidazole (2 eq) in DCM (6 mL/mmol) was added the appropriate hydroxyalkyl derivative of thalidomide (1 eq), and the mixture was stirred for 1 h. The crude product was purified by column chromatography (silica gel, DCM and acetonitrile as eluents) to give the desired product.

### General Procedure for the Alkylation of the Hydroxy Thalidomide

The mixture of 2-(2,6-dioxo-3-piperidyl)-hydroxy-isoindoline-1,3-dione (1 eq), DIPEA (2 eq), and the appropriate □□□-dibromoalkane (3 eq) in DMF (10 mL/mmol) was stirred at 90° C. for 18 h. The crude product was purified by column chromatography (silica gel, heptane and EtOAc as eluents) to give the desired product.

### Degrader Synthesis by Amide Coupling General Procedure

To the product of Preparation 21 (1.5 eq) in dichloromethane (5 mL/mmol) was added N,N-diethylethanamine (15 eq) and [benzotriazol-1-yloxy(dimethylamino)methylene]-dimethyl-ammonium, tetrafluoroborate (1:1) (1.1 eq). Then, the reaction mixture was stirred for 0.5 h. After the treatment of the mixture with the appropriate amine (1 eq), the reaction was stirred to reach the appropriate conversion. The product was purified by preparative HPLC (using acetonitrile and 25 mM aqueous TFA solution as eluents) to give the desired product.

### Degrader Synthesis by Amide Coupling and Hydrolysis General Procedure

To the appropriate acid (2 eq) in dichloromethane (10 mL/mmol) was added N,N-diethylethanamine (7.5 eq) and [benzotriazol-1-yloxy(dimethylamino)methylene]-dimethyl-ammonium, tetrafluoroborate (1:1) (1.1 eq). Then, the reaction mixture was stirred for 0.5 h. After the treatment of the mixture with the appropriate amine (1 eq), the reaction was stirred to reach the appropriate conversion. After the treatment of the mixture with 2,2,2-trifluoroacetic acid (125 eq) in dichloromethane (10 mL/mmol), the reaction was stirred to reach the appropriate conversion. The product was purified by preparative HPLC (using acetonitrile and 25 mM aqueous TFA solution as eluents) to give the desired product.

### General Procedure for the Acylation of Piperidinyl-Isoindolinone

After stirring the appropriate acid derivative (1.5 eq), [dimethylamino(triazolo[4,5-b]pyridin-3-yloxy)methylene]-dimethyl-ammonium, hexafluorophosphate (1:1) (1.1 eq), and N,N-diethylethanamine (5 eq) in dichloromethane (5 mL/mmol) for 20 min, the mixture was treated with 3-[1-oxo-5-(4-piperidyl)isoindolin-2-yl]piperidine-2,6-dione;hydrochloride (1 eq), further stirred until reaching an appropriate conversion, concentrated, and purified by column chromatography to give the desired product.

### General Procedure for the Acylation of Piperidinyl-VHL Ligand

After stirring the appropriate acid (1.5 eq), [dimethylamino(triazolo[4,5-b]pyridin-3-yloxy)methylene]-dimethyl-ammonium, hexafluorophosphate (1:1) (1.1 eq), and N,N-diethylethanamine (5 eq) in dichloromethane (5 mL/mmol) for 20 min, the mixture was treated with Preparation 22 (1 eq), stirred until reaching an appropriate conversion, concentrated, and purified by column chromatography to give the desired product.

### General Procedure for the Alkylation of IAP Ligand

The mixture of tert-butyl N-[(1S)-2-[[(1S)-1-cyclohexyl-2-[(2S)-2-[4-(3-hydroxybenzoyl)thiazol-2-yl]pyrrolidin-1-yl]-2-oxo-ethyl]amino]-1-methyl-2-oxo-ethyl]-N-methyl-carbamate (1 eq), K₂CO₃ (3 eq), and the appropriate α,ω-dibromoalkane (1.1 eq) in MeCN (20 mL/mmol) was stirred at 75° C. for 6 h. The crude product was purified by column chromatography (silica gel, heptane and EtOAc as eluents) or preparative chromatography (using acetonitrile and 25 mM aqueous TFA solution as eluents) to give the desired product.

### General Procedure for the Alkylation of VHL Ligand on Hydroxy Group

The mixture of (2S,4R)-1-[(2S)-2-[(1-fluorocyclopropanecarbonyl)amino]-3,3-dimethyl-butanoyl]-4-hydroxy-N-[[2-hydroxy-4-(4-methylthiazol-5-yl)phenyl]methyl]pyrrolidine-2-carboxamide (1 eq), Cs₂CO₃ (1.1 eq), and the appropriate α,ω-dibromoalkane (2 eq) in DMF (5 mL/mmol) was stirred at 60° C. After reaching an appropriate conversion the product was purified by column chromatography to give the desired product.

### General Procedure for the Alkylation of VHL Ligand on Thiol Group

(2S,4R)-1-[(2R)-2-[(1-fluorocyclopropanecarbonyl)amino]-3-methyl-3-sulfanyl-butanoyl]-4-hydroxy-N-[[4-(4-methylthiazol-5-yl)phenyl]methyl]pyrrolidine-2-carboxamide (1 eq), N-ethyl-N-isopropyl-propan-2-amine (3 eq), and the appropriate α,ω-dibromoalkane (3 eq) in DMF (5 mL/mmol) was stirred at 60° C. After reaching an appropriate conversion the product was purified by column chromatography to give the desired product.

### Degrader Synthesis by Alkylation and Hydrolysis General Procedure

To the product of Preparation 4 and DIPEA (2.0 eq) in MeCN (10 mL/mmol) was added the appropriate alkylating agent (1.5 eq) and the mixture was stirred at 70° C. for 24 h. After cooling to RT, volatiles were removed under reduced pressure. After treating the residue with DCM (15 mL/mmol) and TFA (125 eq) for 1 h, the product was purified by preparative HPLC (using acetonitrile and 0.1% aqueous TFA solution as eluents) to give the desired product.

### Preparation 12: N-[2-[2-[[2-[2-(2,6-dioxo-3-piperidyl)-1,3-dioxo-isoindolin-4-yl]oxyacetyl] amino]ethoxy]ethyl]-2-[methyl(prop-2-ynyl)amino]acetamide

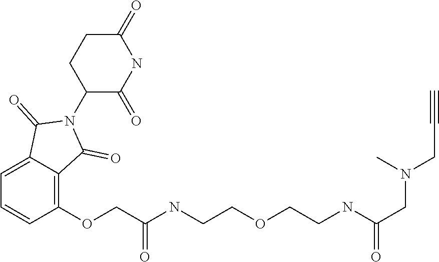

After stirring N-[2-(2-aminoethoxy)ethyl]-2-[2-(2,6-dioxo-3-piperidyl)-1,3-dioxo-isoindolin-4-yl]oxy-acetamide (455 mg, 1 mmol), 2-[methyl(prop-2-ynyl)amino]acetic acid (255 mg, 2 eq) and EDC*HCl (1.15 g, 6 eq) in pyridine (20 mL) for 24 h, the mixture was concentrated and the product was purified by preparative HPLC (Teledyne Method) (C18, 0.2% aqeuous HCOOH, MeCN) to give the desired product (245 mg, 46%).

### General Procedure 1: Synthesis of 12-(((S)-1-((2S,4R)-4-hydroxy-2-(((S)-1-(4-(4-methyl thiazol-5-yl)phenyl)ethyl)carbamoyl)pyrrolidin-1-yl)-3,3-dimethyl-1-oxobutan-2-yl)amino)-12-oxododecanoic acid

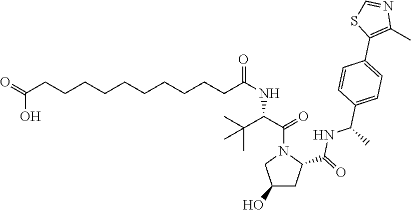 [SMILES: Cc1ncsc1-c1ccc([C@H](C)NC(=O)[C@@H]2C[C@@H](O)CN2C(=O)[C@@H](NC(=O)CCCCCCCCCCC(=O)O)C(C)(C)C)cc1]

To (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (120 mg, 0.270 mmoles) and dodecanedioic acid (311 mg, 1.35 mmoles) in DMF (1.8 mL) was added HATU (113 mg, 0.297 mmoles) and DIPEA (188 uL, 1.08 mmoles). After stirring for 60 minutes, the volatiles were removed in vacuo, the residue was dissolved in DMSO (3 mL) and was purified by RP-HPLC (Teledyne Method) ISCO gold chromatography (10-100% MeCN/H2O, 0.1% NH4OH modifier). Upon lyophilization, 12-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-12-oxododecanoic acid (45 mg, 0.069 mmoles) was obtained. LCMS: MH+=657.6; Rt=2.35 min (5 min acidic method).

### Synthesis of 12-(((S)-1-((2S,4R)-4-hydroxy-2-((2-hydroxy-4-(4-methylthiazol-5-yl)benzyl) carbamoyl)pyrrolidin-1-yl)-3,3-dimethyl-1-oxobutan-2-yl)amino)-12-oxododecanoic acid

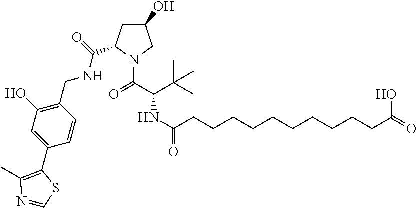 [SMILES: Cc1ncsc1-c1ccc(CNC(=O)[C@@H]2C[C@@H](O)CN2C(=O)[C@@H](NC(=O)CCCCCCCCCCC(=O)O)C(C)(C)C)c(O)c1]

Following General Procedure 1, with (2S,4R)-1-((S)-2-amino-3,3-dimethylbutanoyl)-4-hydroxy-N-(2-hydroxy-4-(4-methylthiazol-5-yl)benzyl)pyrrolidine-2-carboxamide (150 mg, 0.311 mmoles) and dodecanedioic acid (358 mg, 1.55 mmoles), 12-(((S)-1-((2S,4R)-4-hydroxy-2-((2-hydroxy-4-(4-methylthiazol-5-yl)benzyl)carbamoyl)pyrrolidin-1-yl)-3,3-dimethyl-1-oxobutan-2-yl)amino)-12-oxododecanoic acid was obtained. LCMS: MH+=659.6; Rt=1.78 min (5 min acidic method).

### Synthesis of 6-(((S)-1-((2S,4R)-4-hydroxy-2-(((S)-1-(4-(4-methylthiazol-5-yl)phenyl)ethyl) carbamoyl)pyrrolidin-1-yl)-3,3-dimethyl-1-oxobutan-2-yl)carbamoyl)spiro[3.3]heptane-2-carboxylic acid

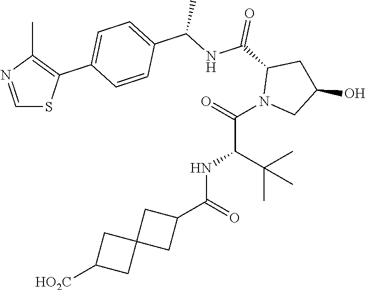 [SMILES: Cc1ncsc1-c1ccc([C@H](C)NC(=O)[C@@H]2C[C@@H](O)CN2C(=O)[C@@H](NC(=O)C2CC3(CC(C(=O)O)C3)C2)C(C)(C)C)cc1]

Following General Procedure 1, with (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (100 mg, 0.19 mmoles) and spiro[3.3]heptane-2,6-dicarboxylic acid (165 mg, 0.89 mmoles), 6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)spiro[3.3]heptane-2-carboxylic acid was obtained. LCMS: MH+=611.7; Rt=1.21 min (5 min acidic method).

### Synthesis of 2-(1-(2-(((S)-1-((2S,4R)-4-hydroxy-2-(((S)-1-(4-(4-methylthiazol-5-yl)phenyl) ethyl)carbamoyl)pyrrolidin-1-yl)-3,3-dimethyl-1-oxobutan-2-yl)amino)-2-oxoethyl)cyclo pentyl)acetic acid

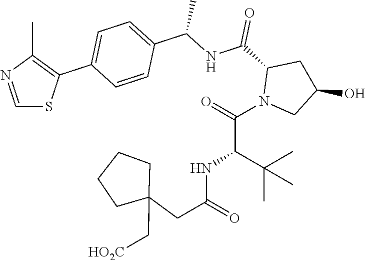 [SMILES: Cc1ncsc1-c1ccc([C@H](C)NC(=O)[C@@H]2C[C@@H](O)CN2C(=O)[C@@H](NC(=O)CC2(CC(=O)O)CCCC2)C(C)(C)C)cc1]

Following General Procedure 1, with (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (100 mg, 0.18 mmoles) and 2,2′-(cyclopentane-1,1-diyl)diacetic acid (167 mg, 0.895 mmoles), 2-(1-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclopentyl)acetic acid was obtained. LCMS: MH+=613.5; Rt=2.01 min (5 min acidic method).

### Synthesis of 9-(((S)-1-((2S,4R)-4-hydroxy-2-(((S)-1-(4-(4-methylthiazol-5-yl)phenyl)ethyl) carbamoyl)pyrrolidin-1-yl)-3,3-dimethyl-1-oxobutan-2-yl)amino)-9-oxononanoic acid

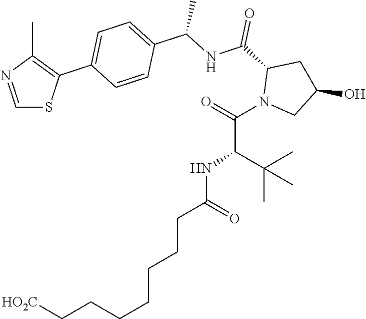 [SMILES: Cc1ncsc1-c1ccc([C@H](C)NC(=O)[C@@H]2C[C@@H](O)CN2C(=O)[C@@H](NC(=O)CCCCCCCC(=O)O)C(C)(C)C)cc1]

Following General Procedure 1, with (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (80 mg, 0.18 mmoles) and nonanedioic acid (169 mg, 0.90 mmoles), 9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoic acid was obtained. LCMS: MH+=615.5; Rt=1.96 min (5 min acidic method).

### Synthesis of 2-((2-(((S)-1-((2S,4R)-4-hydroxy-2-(((S)-1-(4-(4-methylthiazol-5-yl)phenyl) ethyl)carbamoyl)pyrrolidin-1-yl)-3,3-dimethyl-1-oxobutan-2-yl)amino)-2-oxoethyl)thio) acetic acid

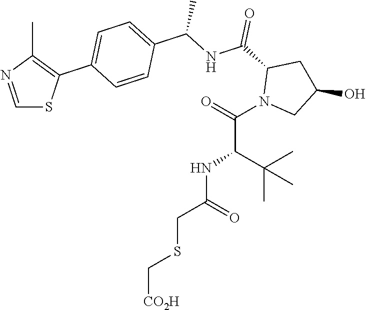 [SMILES: Cc1ncsc1-c1ccc([C@H](C)NC(=O)[C@@H]2C[C@@H](O)CN2C(=O)[C@@H](NC(=O)CSCC(=O)O)C(C)(C)C)cc1]

Following General Procedure 1, with (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (100 mg, 0.225 mmoles) and 2,2′-thiodiacetic acid (169 mg, 1.125 mmoles), 2-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)thio)acetic acid was obtained. LCMS: MH+=577.4; Rt=1.10 min (5 min acidic method).

### Synthesis of N-(2-(((S)-1-((2S,4R)-4-hydroxy-2-(((S)-1-(4-(4-methylthiazol-5-yl)phenyl)ethyl) carbamoyl)pyrrolidin-1-yl)-3,3-dimethyl-1-oxobutan-2-yl)amino)-2-oxoethyl)-N-methyl glycine

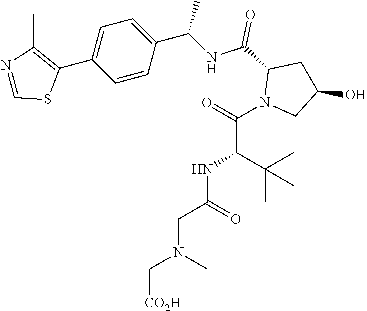 [SMILES: Cc1ncsc1-c1ccc([C@H](C)NC(=O)[C@@H]2C[C@@H](O)CN2C(=O)[C@@H](NC(=O)CN(C)CC(=O)O)C(C)(C)C)cc1]

Following General Procedure 1, with (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (94 mg, 0.21 mmoles) and 2,2′-(methylazanediyl)diacetic acid (143 mg, 0.97 mmoles), N-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)-N-methylglycine was obtained. LCMS: MH+=574.5; Rt=1.06 min (5 min acidic method).

### Synthesis of 5-(((S)-1-((2S,4R)-4-hydroxy-2-(((S)-1-(4-(4-methylthiazol-5-yl)phenyl)ethyl) carbamoyl)pyrrolidin-1-yl)-3,3-dimethyl-1-oxobutan-2-yl)amino)-5-oxopentanoic acid

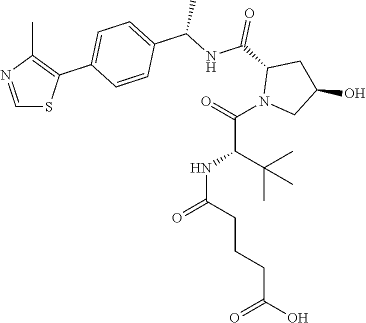 [SMILES: Cc1ncsc1-c1ccc([C@H](C)NC(=O)[C@@H]2C[C@@H](O)CN2C(=O)[C@@H](NC(=O)CCCC(=O)O)C(C)(C)C)cc1]

Following General Procedure 1, with (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (108 mg, 0.243 mmoles) and glutaric acid (160 mg, 1.215 mmoles), 5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoic acid was obtained. LCMS: MH+=559.4; Rt=1.62 min (5 min acidic method).

### Synthesis of 2-((3S,5R)-3-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)adamantan-1-yl)acetic acid

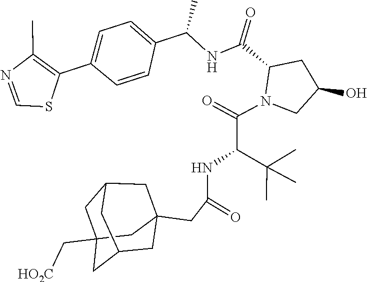 [SMILES: Cc1ncsc1-c1ccc([C@H](C)NC(=O)[C@@H]2C[C@@H](O)CN2C(=O)[C@@H](NC(=O)CC23CC4CC(CC(CC(=O)O)(C4)C2)C3)C(C)(C)C)cc1]

Following General Procedure 1, with (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (100 mg, 0.18 mmoles) and 2,2′-((1 s,3s,5r,7r)-adamantane-1,3-diyl)diacetic acid (226 mg, 0.90 mmoles), 2-((3S,5R)-3-(2-(((S)-1-((2S,4R)-4-hydroxy-2-(((S)-1-(4-(4-methylthiazol-5-yl)phenyl)ethyl) carbamoyl)pyrrolidin-1-yl)-3,3-dimethyl-1-oxobutan-2-yl)amino)-2-oxoethyl)adamantan-1-yl)acetic acid was obtained. LCMS: MH+=679.9; Rt=2.24 min (5 min acidic method).

### Synthesis of (2S,4R)-4-hydroxy-1-((S)-2-(12-hydroxydodecanamido)-3,3-dimethyl butanoyl)-N—((S)-1-(4-(4-methylthiazol-5-yl)phenyl)ethyl)pyrrolidine-2-carboxamide

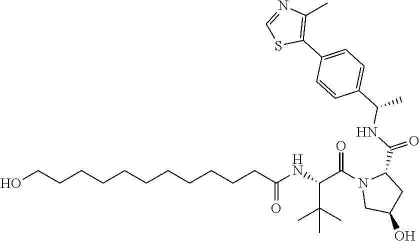 [SMILES: Cc1ncsc1-c1ccc([C@H](C)NC(=O)[C@@H]2C[C@@H](O)CN2C(=O)[C@@H](NC(=O)CCCCCCCCCCCO)C(C)(C)C)cc1]

Following General Procedure 1, with 12-hydroxydodecanoic acid (63.9 mg, 0.295 mmoles) and (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (150 mg, 0.269 mmoles), (2S,4R)-4-hydroxy-1-((S)-2-(12-hydroxydodecanamido)-3,3-dimethylbutanoyl)-N—((S)-1-(4-(4-methylthiazol-5-yl)phenyl)ethyl)pyrrolidine-2-carboxamide was obtained. LCMS: MH+=643.8; Rt=2.43 min (5 min acidic method).

### General Procedure 2: Synthesis of (2S,4R)-1-((S)-3,3-dimethyl-2-(12-oxododecanamido) butanoyl)-4-hydroxy-N—((S)-1-(4-(4-methylthiazol-5-yl)phenyl)ethyl)pyrrolidine-2-carboxamide

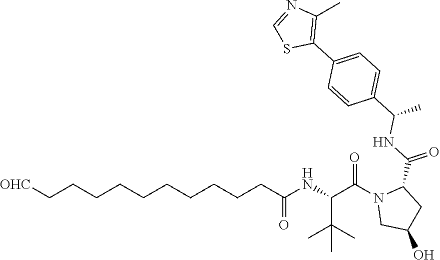 [SMILES: Cc1ncsc1-c1ccc([C@H](C)NC(=O)[C@@H]2C[C@@H](O)CN2C(=O)[C@@H](NC(=O)CCCCCCCCCCC=O)C(C)(C)C)cc1]

To (2S,4R)-4-hydroxy-1-((S)-2-(12-hydroxydodecanamido)-3,3-dimethylbutanoyl)-N—((S)-1-(4-(4-methylthiazol-5-yl)phenyl)ethyl)pyrrolidine-2-carboxamide (135 mg, 0.21 mmoles) in CH2Cl2 (1.5 mL) was added Dess Martin Periodinane (98 mg, 0.23 mmoles). After stirring for 15 hours the volatiles were removed in vacuo, the residue was dissolved in DMSO (4 mL) and was purified by RP-HPLC (Teledyne Method) ISCO gold chromatography (10-100% MeCN/H2O, 0.1% NH4OH modifier). Upon lyophilization, (2S,4R)-1-((S)-3,3-dimethyl-2-(12-oxododecanamido)butanoyl)-4-hydroxy-N—((S)-1-(4-(4-methylthiazol-5-yl)phenyl)ethyl)pyrrolidine-2-carboxamide (75 mg, 0.116 mmoles) was obtained. LCMS: MH+=641.6; Rt=2.45 min (5 min acidic method).

### Synthesis of (2S,4R)-1-((S)-3,3-dimethyl-2-(9-oxononanamido)butanoyl)-4-hydroxy-N—((S)-1-(4-(4-methylthiazol-5-yl)phenyl)ethyl)pyrrolidine-2-carboxamide

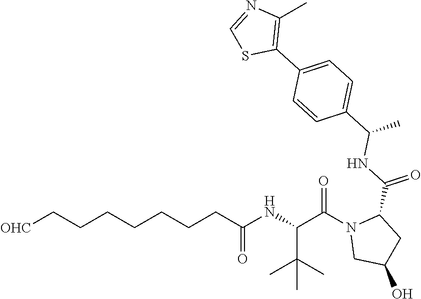 [SMILES: Cc1ncsc1-c1ccc([C@H](C)NC(=O)[C@@H]2C[C@@H](O)CN2C(=O)[C@@H](NC(=O)CCCCCCCC=O)C(C)(C)C)cc1]

Following General Procedure 1, with (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (155 mg, 0.277 mmoles) and 9-hydroxynonanoic acid (53.2 mg, 0.305 mmoles), followed by General Procedure 2, (2S,4R)-1-((S)-3,3-dimethyl-2-(9-oxononanamido)butanoyl)-4-hydroxy-N—((S)-1-(4-(4-methyl thiazol-5-yl)phenyl)ethyl)pyrrolidine-2-carboxamide was obtained. LCMS: MH+=599.7; Rt=2.17 min (5 min acidic method).

### Synthesis of (2S,4R)-1-((S)-2-(1-fluorocyclopropane-1-carboxamido)-3,3-dimethyl butanoyl)-4-hydroxy-N-(4-(4-methylthiazol-5-yl)-2-(2-oxo-2-((5-oxopentyl)amino)ethoxy) benzyl)pyrrolidine-2-carboxamide

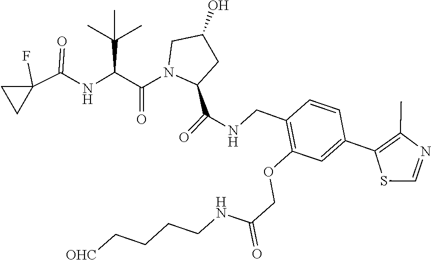 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCC=O)c1]

Following General Procedure 1, with 2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)acetic acid (200 mg, 0.34 mmoles) and 5-aminopentan-1-ol (34.9 mg, 0.34 mmoles), followed by General Procedure 2, (2S,4R)-1-((S)-2-(1-fluorocyclopropane-1-carboxamido)-3,3-dimethylbutanoyl)-4-hydroxy-N-(4-(4-methylthiazol-5-yl)-2-(2-oxo-2-((5-oxopentyl)amino)ethoxy)benzyl)pyrrolidine-2-carboxamide was obtained. LCMS: M+Na+=696.7; Rt=1.83 min (5 min acidic method).

### Synthesis of (2S,4R)-1-((S)-2-(1-fluorocyclopropane-1-carboxamido)-3,3-dimethylbutanoyl)-4-hydroxy-N-(2-(2-(methyl(5-oxopentyl)amino)-2-oxoethoxy)-4-(4-methylthiazol-5-yl)benzyl)pyrrolidine-2-carboxamide

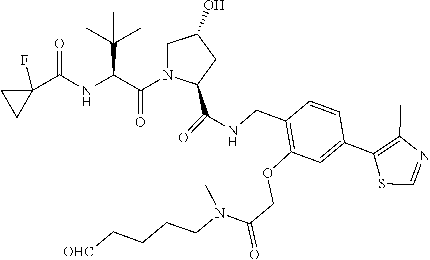 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)N(C)CCCCC=O)c1]

Following General Procedure 1, with 2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)acetic acid (126 mg, 0.213 mmoles) and 5-(methylamino)pentan-1-01 (25 mg, 0.231 mmoles), followed by General Procedure 1, (2S,4R)-1-((S)-2-(1-fluorocyclopropane-1-carboxamido)-3,3-dimethylbutanoyl)-4-hydroxy-N-(2-(2-(methyl(5-oxopentyl)amino)-2-oxoethoxy)-4-(4-methylthiazol-5-yl)benzyl)pyrrolidine-2-carboxamide was obtained. LCMS: MH+=688.8; Rt=1.88 min (5 min acidic method).

### General Procedure 3: Synthesis of (2S,4R)-1-((S)-2-(1-fluorocyclopropane-1-carboxamido)-3,3-dimethylbutanoyl)-4-hydroxy-N-(2-((9-hydroxynonyl)oxy)-4-(4-methyl thiazol-5-yl)benzyl)pyrrolidine-2-carboxamide

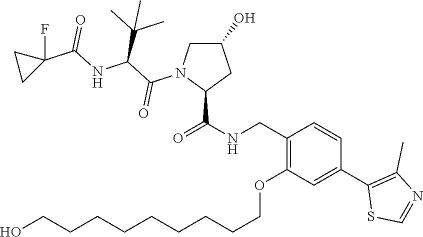 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCO)c1]

To a mixture of (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (300 mg, 0.563 mmoles) and potassium carbonate (78 mg, 0.563 mmoles) in DMF (2.5 mL) was added 9-bromononan-1-ol (251 mg, 1.13 mmoles) and a speck of KI. After stirring at 90° C. for 5 hours, cooling to rt and neutralizing by addition of 1 N HCl, the volatiles were removed in vacuo. The residue was purified by SiO2 chromatography (0-10% MeOH/CH2Cl2) to yield (2S,4R)-1-((S)-2-(1-fluorocyclopropane-1-carboxamido)-3,3-dimethylbutanoyl)-4-hydroxy-N-(2-((9-hydroxynonyl)oxy)-4-(4-methylthiazol-5-yl)benzyl)pyrrolidine-2-carboxamide (300 mg, 0.44 mmoles). LCMS: MH+=675.8; Rt=2.50 min (5 min acidic method).

### Synthesis of (2S,4R)-1-((S)-2-(1-fluorocyclopropane-1-carboxamido)-3,3-dimethyl butanoyl)-4-hydroxy-N-(4-(4-methylthiazol-5-yl)-2-((9-oxononyl)oxy)benzyl)pyrrolidine-2-carboxamide

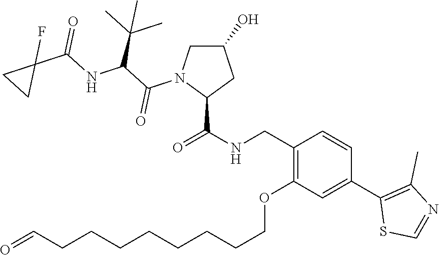

Following General Procedure 2, with (2S,4R)-1-((S)-2-(1-fluorocyclopropane-1-carbox amido)-3,3-dimethylbutanoyl)-4-hydroxy-N-(2-((9-hydroxynonyl)oxy)-4-(4-methylthiazol-5-yl)benzyl)pyrrolidine-2-carboxamide (300 mg, 0.445 mmoles), (2S,4R)-1-((S)-2-(1-fluorocyclo propane-1-carboxamido)-3,3-dimethylbutanoyl)-4-hydroxy-N-(4-(4-methylthiazol-5-yl)-2-((9-oxononyl)oxy)benzyl)pyrrolidine-2-carboxamide was obtained. LCMS: MH+=673.7; Rt=2.57 min (5 min acidic method).

### Synthesis of (2S,4R)-1((S)-2-(1-fluorocyclopropane-1-carboxamido)-3,3-dimethyl butanoyl)-4-hydroxy-N-(4-(4-methylthiazol-5-yl)-2((8-oxooctyl)oxy)benzyl)pyrrolidine-2-carboxamide

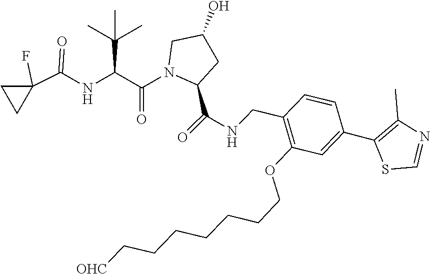 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCC=O)c1]

Following General Procedure 3, with (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (400 mg, 0.75 mmoles) and 8-bromooctan-1-ol (314 mg, 1.50 mmoles), followed by General Procedure 2, (2S,4R)-1-((S)-2-(1-fluorocyclopropane-1-carboxamido)-3,3-dimethylbutanoyl)-4-hydroxy-N-(4-(4-methylthiazol-5-yl)-2-((8-oxooctyl)oxy)benzyl)pyrrolidine-2-carboxamide was obtained. LCMS: MH+=659.8; Rt=2.49 min (5 min acidic method).

### Synthesis of (2S,4R)-1-((S)-2-(1-fluorocyclopropane-1-carboxamido)-3,3-dimethylbutanoyl)-4-hydroxy-N-(4-(4-methylthiazol-5-yl)-2-((12-oxododecyl)oxy)benzyl)pyrrolidine-2-carboxamide

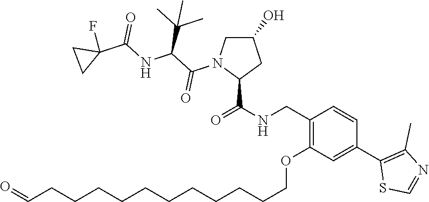 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCCC=O)c1]

Following General Procedure 3, with (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (300 mg, 0.563 mmoles) and 12-bromododecan-1-ol (299 mg, 1.126 mmoles), followed by General Procedure 2, (2S,4R)-1-((S)-2-(1-fluorocyclopropane-1-carboxamido)-3,3-dimethylbutanoyl)-4-hydroxy-N-(4-(4-methylthiazol-5-yl)-2-((12-oxododecyl)oxy)benzyl)pyrrolidine-2-carboxamide was obtained. LCMS: MH+=715.9; Rt=3.09 min (5 min acidic method).

### Synthesis of (2S,4R)-1-((S)-2-(1-fluorocyclopropane-1-carboxamido)-3,3-dimethylbutanoyl)-N-(2-((4-formylbenzyl)oxy)-4-(4-methylthiazol-5-yl)benzyl)-4-hydroxypyrrolidine-2-carboxamide

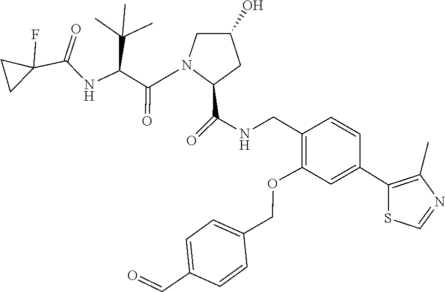 [SMILES: Cc1ncsc1-c1ccc(CNC(=O)[C@@H]2C[C@@H](O)CN2C(=O)[C@@H](NC(=O)C2(F)CC2)C(C)(C)C)c(OCc2ccc(C=O)cc2)c1]

Following General Procedure 3, with (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (225 mg, 0.422 mmoles) and 4-(chloromethyl)benzaldehyde (131 mg, 0.845 mmoles), (2S,4R)-1-((S)-2-(1-fluorocyclopropane-1-carboxamido)-3,3-dimethylbutanoyl)-N-(2-((4-formylbenzyl)oxy)-4-(4-methylthiazol-5-yl)benzyl)-4-hydroxypyrrolidine-2-carboxamide was obtained. LCMS: MH+=651.6; Rt=2.14 min (5 min acidic method).

### Synthesis of 12-(((S)-1-((2S,4R)-4-hydroxy-2-((4-(4-methylthiazol-5-yl)benzyl)carbamoyl) pyrrolidin-1-yl)-3,3-dimethyl-1-oxobutan-2-yl)amino)-12-oxododecanoic acid

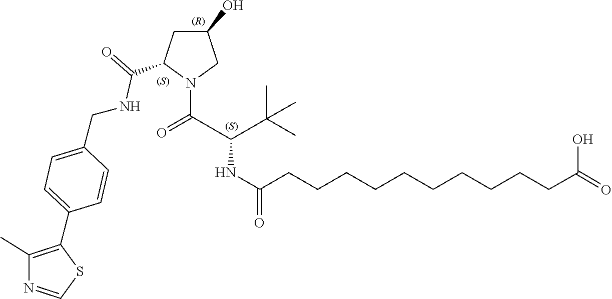

Following General Procedure 1, with (2S,4R)-1-((S)-2-amino-3,3-dimethylbutanoyl)-4-hydroxy-N-(4-(4-methylthiazol-5-yl)benzyl)pyrrolidine-2-carboxamide (140 mg, 0.30 mmoles) and dodecanedioic acid (276 mg, 1.2 mmoles), 12-(((S)-1-((2S,4R)-4-hydroxy-2-((4-(4-methyl thiazol-5-yl)benzyl)carbamoyl)pyrrolidin-1-yl)-3,3-dimethyl-1-oxobutan-2-yl)amino)-12-oxo dodecanoic acid was obtained. LCMS: MH+=643.7; Rt=2.20 min (5 min acidic method).

### Synthesis of 3-fluoro-4-(((S)-1-((2S,4R)-4-hydroxy-2-(((S)-1-(4-(4-methylthiazol-5-yl)phenyl) ethyl)carbamoyl)pyrrolidin-1-yl)-3,3-dimethyl-1-oxobutan-2-yl)carbamoyl) benzoic acid

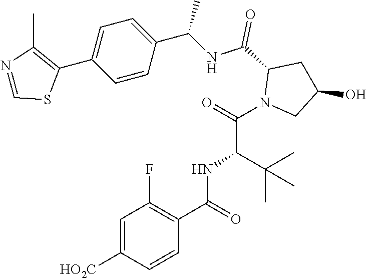 [SMILES: Cc1ncsc1-c1ccc([C@H](C)NC(=O)[C@@H]2C[C@@H](O)CN2C(=O)[C@@H](NC(=O)c2ccc(C(=O)O)cc2F)C(C)(C)C)cc1]

Following General Procedure 1, with (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (150 mg, 0.269 mmoles) and 2-fluoro-4-(methoxycarbonyl)benzoic acid (58.5 mg, 295 mmoles) the corresponding amide was obtained. This amide was dissolved in 1:1 THF/H2O (2 mg) and LiOH (36.9 mg, 1.537 mmoles) was added. After stirring for 5 hours the solution was neutralized by addition of 1 N HCl, the volatiles were removed in vacuo and the residue was purified by RP-HPLC (Teledyne Method) (10-100% MeCN/H2O, 0.1% TFA modifier). After lyophilization, 3-fluoro-4-(((S)-1-((2S,4R)-4-hydroxy-2-(((S)-1-(4-(4-methylthiazol-5-yl)phenyl)ethyl)carbamoyl) pyrrolidin-1-yl)-3,3-dimethyl-1-oxobutan-2-yl)carbamoyl)benzoic acid was obtained. LCMS: MH+=611.7; Rt=1.96 min (5 min acidic method).

### Synthesis of 5-(2-(2-(((2S,4R)-1-((S)-2-(1-fluorocyclopropane-1-carboxamido)-3,3-dimethyl butanoyl)-4-hydroxypyrrolidine-2-carboxamido)methyl)-5-(4-methylthiazol-5-yl)phenoxy) acetamido)pentanoic acid

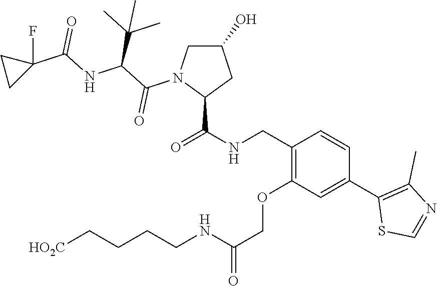 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)NCCCCC(=O)O)c1]

Following General Procedure 1, with 2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)acetic acid (76 mg, 0.129 mmoles) and Ethyl 5-amino-valerate hydrochloride (25.6 mg, 142 mmoles), the corresponding amide was obtained. This amide was dissolved in 1:1 THF/H2O (2 mg) and LiOH (26.7 mg, 1.114 mmoles) was added. After stirring for 5 hours the solution was neutralized by addition of 1 N HCl, the volatiles were removed in vacuo and the residue was purified by RP-HPLC (Teledyne Method) (10-100% MeCN/H2O, 0.1% TFA modifier). After lyophilization, 5-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)acetamido)pentanoic acid was obtained. LCMS: MH+=690.7; Rt=1.75 min (5 min acidic method).

### Synthesis of 4-(((S)-1-((2S,4R)-4-hydroxy-2-(((S)-1-(4-(4-methylthiazol-5-yl)phenyl)ethyl) carbamoyl)pyrrolidin-1-yl)-3,3-dimethyl-1-oxobutan-2-yl)amino)-4-oxobutanoic acid

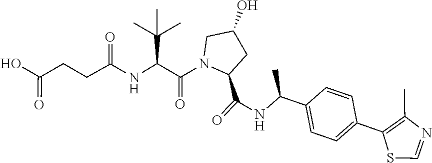 [SMILES: Cc1ncsc1-c1ccc([C@H](C)NC(=O)[C@@H]2C[C@@H](O)CN2C(=O)[C@@H](NC(=O)CCC(=O)O)C(C)(C)C)cc1]

To a solution of (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (4.21 grams, 9.48 mmoles) in THF (50 mL) was added DIPEA (3.64 mL, 20.85 mmoles) followed by succinic anhydride (949 mg, 9.48 mmoles). After stirring overnight the volatiles were removed in vacuo, the residue was dissolved in DMSO and was purified by RP-ISCO (10-100% MeCN/H2O, 0.1% formic acid modifier) to yield after lyophilization 4-(((S)-1-((2S,4R)-4-hydroxy-2-(((S)-1-(4-(4-methylthiazol-5-yl)phenyl)ethyl) carbamoyl)pyrrolidin-1-yl)-3,3-dimethyl-1-oxobutan-2-yl)amino)-4-oxobutanoic acid. LCMS: MH+=545.5; Rt=1.56 min (5 min acidic method).

### Synthesis of methyl 2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)acetate

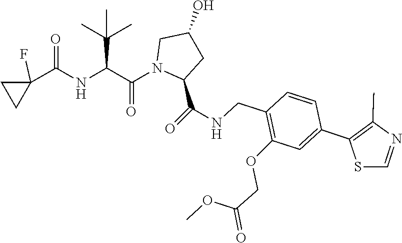 [SMILES: COC(=O)COc1cc(-c2scnc2C)ccc1CNC(=O)[C@@H]1C[C@@H](O)CN1C(=O)[C@@H](NC(=O)C1(F)CC1)C(C)(C)C]

To a solution of (2S,4R)-1-((S)-2-(1-fluorocyclopropane-1-carboxamido)-3,3-dimethyl butanoyl)-4-hydroxy-N-(2-hydroxy-4-(4-methylthiazol-5-yl)benzyl)pyrrolidine-2-carboxamide (5.48 grams, 10.29 mmoles) in acetone was added and potassium carbonate (3.41 grams, 24.7 mmoles) and then methyl 2-bromoacetate (1.32 mL, 13.89 mmoles). After stirring for 20 hours, the volatiles were removed in vacuo and the residue was partitioned between EtOAc and H2O, washed with brined, dried over MgSO4, filtered, concentrated and purified by SiO2 chromatography (0-15% MeOH/CH2Cl2) to yield methyl 2-(2-(((2S,4R)-1-((S)-2-(1-fluorocyclo propane-1-carboxamido)-3,3-dimethylbutanoyl)-4-hydroxypyrrolidine-2-carboxamido)methyl)-5-(4-methylthiazol-5-yl)phenoxy)acetate (4.80 grams, 7.86 mmoles). LCMS: MH+=605.9; Rt=1.76 min (5 min acidic method).

### Synthesis of 2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)acetic acid

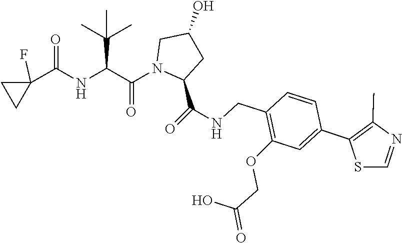 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)O)c1]

To a solution of methyl 2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl) phenoxy)acetate (4.80 grams, 7.94 mmoles) in 2:1 THF/MeOH (60 mL) was added 1 N LiOH (8.73 mL, 8.73 mmoles). After stirring for 20 hours the solution was neutralized by addition of 1 N HCl and the volatiles were removed in vacuo, DMSO (20 mL) was added and the solution was purified by RP-HPLC (Teledyne Method) (10-100% MeCN/H2O with 0.1% TFA modifier). After lyophilization, 2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)acetic acid was obtained. LCMS: MH+=591.5; Rt=1.63 min (5 min acidic method).

### Synthesis of (2S,4R)—N-(2-(allyloxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

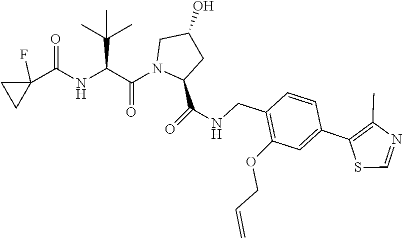 [SMILES: C=CCOc1cc(-c2scnc2C)ccc1CNC(=O)[C@@H]1C[C@@H](O)CN1C(=O)[C@@H](NC(=O)C1(F)CC1)C(C)(C)C]

To a solution of (2S,4R)-1-((S)-2-(1-fluorocyclopropane-1-carboxamido)-3,3-dimethyl butanoyl)-4-hydroxy-N-(2-hydroxy-4-(4-methylthiazol-5-yl)benzyl)pyrrolidine-2-carboxamide (2.67 grams, 5.01 mmoles) in acetone was added and potassium carbonate (3.41 grams, 24.7 mmoles) and then allylbromide (0.67 mL, 7.74 mmoles). After stirring for 20 hours, the volatiles were removed in vacuo and the residue was partitioned between EtOAc and H2O, washed with brined, dried over MgSO4, filtered, concentrated and purified by SiO2 chromatography (0-15% MeOH/CH2Cl2) to yield (2S,4R)—N-(2-(allyloxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (2.21 grams, 3.86 mmoles). LCMS: MH+=573.6; Rt=2.07 min (5 min acidic method).

### Synthesis of (2S,4R)-1-((S)-2-(1-fluorocyclopropane-1-carboxamido)-3,3-dimethyl butanoyl)-4-hydroxy-N-(4-(4-methylthiazol-5-yl)-2-(2-oxoethoxy)benzyl)pyrrolidine-2-carboxamide

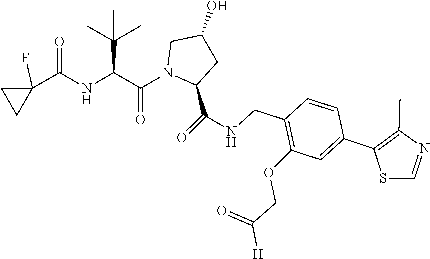 [SMILES: Cc1ncsc1-c1ccc(CNC(=O)[C@@H]2C[C@@H](O)CN2C(=O)[C@@H](NC(=O)C2(F)CC2)C(C)(C)C)c(OCC=O)c1]

To an ice cold solution of (2S,4R)—N-(2-(allyloxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (2.21 grams, 3.86 mmoles) in 3:1 acetone/H2O (50 mL) was added a solution of potassium permanganate (0.671 grams, 4.24 mmoles) in 3:1 acetone/H2O (50 mL). The ice bath was removed and the solution stirred for 3 hours at which time the mixture was filtered through a pad of celite and the acetone was removed in vacuo. THF (50 mL) was added to the aqueous solution followed by sodium periodate (1.65 grams, 7.72 mmoles). After stirring for 22 hours the mixture was filtered through a pad of celite and the volatiles were removed in vacuo. DMSO was added to the aqueous solution and the material was purified by ISCO RP-HPLC (20-60% MeCN/H2O, with 0.1% TFA modifier) to yield after lyophilization (2S,4R)-1-((S)-2-(1-fluorocyclopropane-1-carboxamido)-3,3-dimethylbutanoyl)-4-hydroxy-N-(4-(4-methylthiazol-5-yl)-2-(2-oxoethoxy)benzyl)pyrrolidine-2-carboxamide (1.58 grams, 2.73 mmole). LCMS: MH+=575.5; Rt=1.53 min (5 min acidic method).

### C. Synthesis and Characterization of Bifunctional Bcl-xL Degrader Compounds

Exemplary bifunctional Bcl-xL degrader compounds, comprising a Bcl-xL payload covalently linked to a degrading compound (DSM), were synthesized using exemplary methods described in this example.

### General Procedures

### General Procedure for Production of VHL Ligand-Based Degraders Via Alkylation

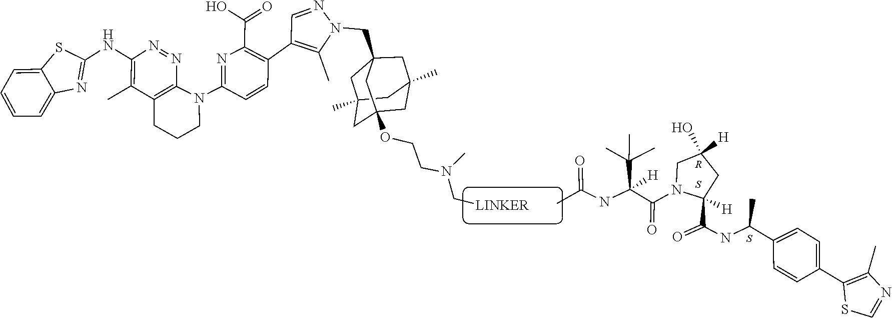

To the product of Preparation 4 and DIPEA (1.0 mL/mmol) in a mixture of MeCN (10 mL/mmol) and 1-methyl-2-pyrrolidinone (10 mL/mmol) was added the appropriate alkylating agent (1.5 eq) and the mixture was stirred at 70° C. until appropriate conversion was achieved. After cooling to RT, the reaction was treated with a 10% aqeuous KOH solution (6 eq) and the mixture was stirred at 40° C. until appropriate conversion was achieved. The product was purified by preparative HPLC (using acetonitrile and 0.1% aqueous TFA solution as eluents) to give the desired product.

### General Procedure for Production of Degraders Via Acylation

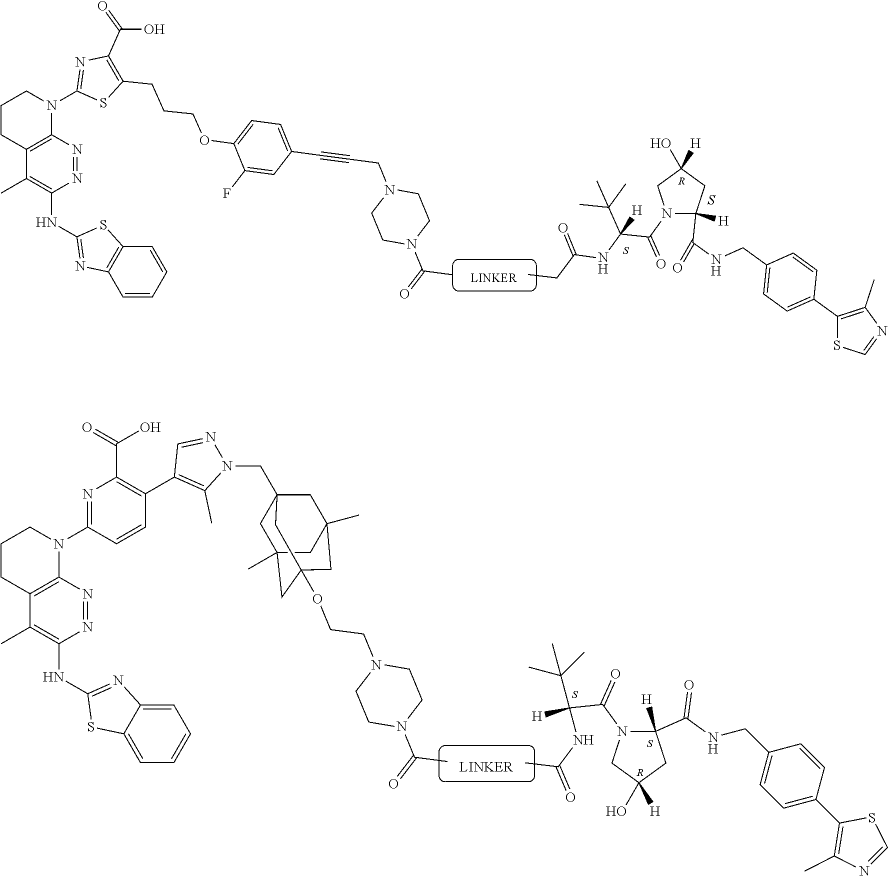

wherein X represents an oxygen atom or —NH— group.

After stirring the appropriate carboxylic acid (1.5 eq), TEA (10 eq) and HATU (1.7 eq) in DMF (10 mL/mmol) at 50° C. for 20 min, Preparation 5 or Preparation 9 was added, and stirred at 50° C. for 1 h. The product was purified by preparative HPLC (Teledyne EZ) (C18, using acetonitrile and 0.1% aqueous TFA solution as eluents) to give the desired product.

### General Procedure for the Production of Thalidomide-Based Degraders Via Alkylation

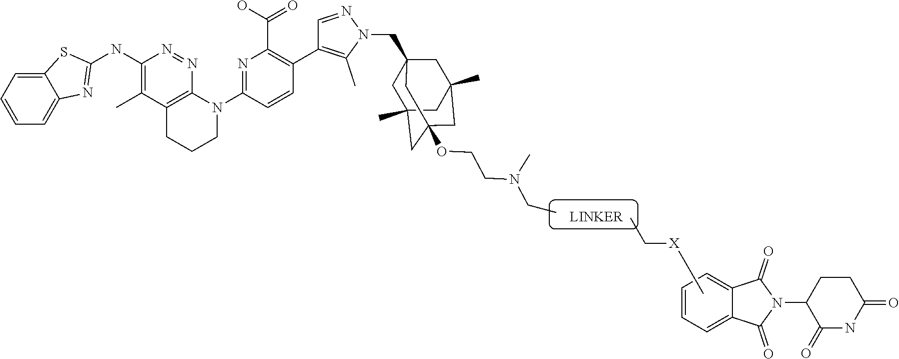

To the product of Preparation 4 and DIPEA (1.0 mL/mmol) in MeCN (20 mL/mmol) was added the appropriate alkylating agent (1.6 eq) and the mixture was stirred at 70° C. until appropriate conversion was achieved. After concentration, to the residue was added DCM (45 mL/mmol) and TFA (45 mL/mmol) at 0° C. The reaction was stirred at 40° C. until appropriate conversion was achieved. The product was purified by preparative HPLC (Teledyne EZ) (C18, using acetonitrile and 0.1% aqueous TFA solution as eluents) to give the desired product.

### Example 1: 6-[3-(1,3-benzothiazol-2-ylamino)-4-methyl-6,7-dihydro-5H-pyrido[2,3-c]pyridazin-8-yl]-3-[1-[[3-[2-[[16-[[(1S)-1-[(2S,4R)-4-hydroxy-2-[[(1S)-1-[4-(4-methyl thiazol-5-yl)phenyl]ethyl]carbamoyl]pyrrolidine-1-carbonyl]-2,2-dimethyl-propyl]amino]-16-oxo-hexadecyl]-methyl-amino]ethoxy]-5,7-dimethyl-1-adamantyl] methyl]-5-methyl-pyrazol-4-yl]pyridine-2-carboxylic acid

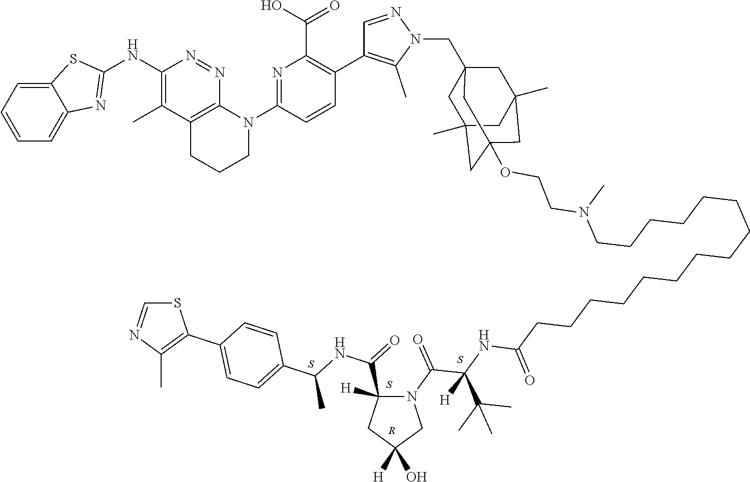

### Step A: (2S,4R)-4-hydroxy-1-[(2S)-2-(16-hydroxyhexadecanoylamino)-3,3-dimethyl-butanoyl]-N-[(1S)-1-[4-(4-methylthiazol-5-yl)phenyl]ethyl]pyrrolidine-2-carboxamide

Using General procedure for the acylation of VHL ligands starting from (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide, hydrogen chloride (1:1) (0.42 mmol) and 16-hydroxyhexadecanoic-acid as the appropriate acid, 93 mg of the desired product was obtained. ¹H NMR (500 MHz, DMSO-d₆): δ ppm 8.98 (s, 1H), 8.38 (d, 1H), 7.79 (d, 1H), 7.43 (d, 2H), 7.37 (d, 2H), 5.10 (d, 1H), 4.91 (m, 1H), 4.51 (d, 1H), 4.41 (t, 1H), 4.32 (t, 1H), 4.27 (m, 1H), 3.64-3.55 (m, 2H), 3.36 (q, 2H), 2.45 (s, 3H), 2.24/2.09 (m+m, 2H), 2.00/1.78 (m+m, 2H), 1.52-1.38 (m, 2H), 1.38 (q, 2H), 1.37 (d, 3H), 1.29-1.19 (m, 22H), 0.93 (s, 9H); ¹³C NMR (125 MHz, DMSO-d₆) δ ppm 172.5, 171.1, 170.1, 152.0, 129.3, 126.8, 69.2, 61.2, 59.0, 56.8, 56.7, 48.2, 38.2, 35.3, 33, 26.9, 26.0, 22.9, 16.5; HRMS-ESI (m/z):[M+H]+ calcd for C₃₉H₆₃N₄O₅S: 699.4519. found: 699.4514.

### Step B: [16-[[(1S)-1-[(2S,4R)-4-hydroxy-2-[[(1S)-1-[4-(4-methylthiazol-5-yl)phenyl] ethyl]carbamoyl]pyrrolidine-1-carbonyl]-2,2-dimethyl-propyl]amino]-16-oxo-hexadecyl]4-methylbenzenesulfonate

Using the General procedure for the tosylation of the hydroxyalkyl VHL ligand-derivatives starting from the product of Step A (90 mg), 49 mg of the desired product was obtained. ¹H NMR (500 MHz, DMSO-d₆): δ ppm 8.98 (s, 1H), 8.38 (d, 1H), 7.79 (d, 1H), 7.78 (dm, 2H), 7.48 (dm, 2H), 7.43 (d, 2H), 7.37 (d, 2H), 5.10 (d, 1H), 4.91 (m, 1H), 4.51 (d, 1H), 4.41 (t, 1H), 4.27 (m, 1H), 3.99 (t, 2H), 3.64-3.55 (m, 2H), 2.45 (s, 3H), 2.42 (s, 3H), 2.24/2.09 (m+m, 2H), 2.00/1.78 (m+m, 2H), 1.53 (m, 2H), 1.52-1.38 (m, 2H), 1.37 (d, 3H), 1.3-1.1 (br., 22H), 0.93 (s, 9H); ¹³C NMR (125 MHz, DMSO-d₆) δ ppm 172.5, 171.1, 170.1, 152, 130.6, 129.3, 128.0, 126.8, 71.4, 69.2, 59.0, 56.8, 56.7, 48.2, 38.2, 35.3, 28.6, 26.9, 26.0, 22.9, 21.6, 16.5; HRMS-ESI (m/z):[M+H]⁺ calcd for C₄₆H₆₉N₄O₇S₂: 853.4608. found: 853.4602.

### Step C: 6-[3-(1,3-benzothiazol-2-ylamino)-4-methyl-6,7-dihydro-5H-pyrido[2,3-c]pyridazin-8-yl]-3-[1-[[3-[2-[[16-[[(1S)-1-[(2S,4R)-4-hydroxy-2-[[(1S)-1-[4-(4-methylthiazol-5-yl)phenyl] ethyl]carbamoyl]pyrrolidine-1-carbonyl]-2,2-dimethyl-propyl]amino]-16-oxo-hexadecyl]-methyl-amino]ethoxy]-5,7-dimethyl-1-adamantyl]methyl]-5-methyl-pyrazol-4-yl]pyridine-2-carboxylic acid

Using the General procedure for production of VHL ligand-based degraders via alkylation starting from the product of Preparation 4 (30 mg) and the product of Step B as the appropriate alkylating agent, 14 mg of the desired product was obtained. HRMS-ESI (m/z):[M+2H]²⁺ calcd for C₈₀H₁₁₁N₁₃O₇S₂: 714.9086. found: 714.9078.

### Example 2: 6-[3-(1,3-benzothiazol-2-ylamino)-4-methyl-6,7-dihydro-5H-pyrido[2,3-c]pyridazin-8-yl]-3-[1-[[3-[2-[[12-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-12-oxo-dodecyl]-methyl-amino]ethoxy]-5,7-dimethyl-1-adamantyl]methyl]-5-methyl-pyrazol-4-yl]pyridine-2-carboxylic acid

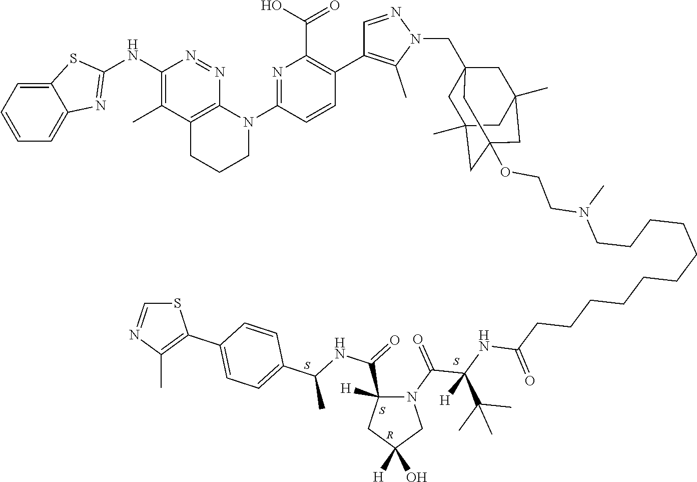

### Step A: (2S,4R)-4-hydroxy-1-[(2S)-2-(12-hydroxydodecanoylamino)-3,3-dimethyl-butanoyl]-N-[(1S)-1-[4-(4-methylthiazol-5-yl)phenyl]ethyl]pyrrolidine-2-carboxamide

Using General procedure for the acylation of VHL ligands starting from (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide, hydrogen chloride (1:1) (0.42 mmol) and 12-hydroxydodecanoic acid as the appropriate acid, 115 mg of the desired product was obtained. MS-ESI (m/z): 644 [M+H]⁺.

### Step B: [12-[[(1S)-1-[(2S,4R)-4-hydroxy-2-[[(1S)-1-[4-(4-methylthiazol-5-yl)phenyl] ethyl]carbamoyl]pyrrolidine-1-carbonyl]-2,2-dimethyl-propyl]amino]-12-oxo-dodecyl]4-methyl benzenesulfonate

Using the General procedure for the tosylation of the hydroxyalkyl VHL ligand-derivatives starting from the product of Step A (110 mg), 100 mg of the desired product was obtained. ¹H NMR (500 MHz, DMSO-d₆): δ ppm 8.98 (s, 1H), 8.38 (d, 1H), 7.79 (d, 1H), 7.78 (dm, 2H), 7.48 (dm, 2H), 7.43 (d, 2H), 7.37 (d, 2H), 5.10 (br., 1H), 4.91 (m, 1H), 4.51 (d, 1H), 4.41 (t, 1H), 4.27 (br., 1H), 4.00 (t, 2H), 3.64-3.55 (m, 2H), 2.45 (s, 3H), 2.42 (s, 3H), 2.24/2.09 (m+m, 2H), 2.00/1.78 (m+m, 2H), 1.53 (m, 2H), 1.52-1.38 (m, 2H), 1.37 (d, 3H), 1.3-1.05 (br., 14H), 0.93 (s, 9H); ¹³C NMR (125 MHz, DMSO-d₆) δ ppm 172.5, 171.1, 170.1, 152.0, 130.6, 129.3, 128.0, 126.8, 71.4, 69.2, 59.0, 56.8, 56.7, 48.2, 38.2, 35.3, 28.6, 26.9, 26.0, 22.9, 21.6, 16.5; HRMS-ESI (m/z):[M+H]⁺ calcd for C₄₂H₆₁N₄O₇S₂: 797.3982. found: 797.3977.

### Step C: 6-[3-(1,3-benzothiazol-2-ylamino)-4-methyl-6,7-dihydro-5H-pyrido[2,3-c]pyridazin-8-yl]-3-[1-[[3-[2-[[12-[[(1S)-1-[(2S,4R)-4-hydroxy-2-[[(1S)-1-[4-(4-methylthiazol-5-yl)phenyl] ethyl]carbamoyl]pyrrolidine-1-carbonyl]-2,2-dimethyl-propyl]amino]-12-oxo-dodecyl]-methyl-amino]ethoxy]-5,7-dimethyl-1-adamantyl]methyl]-5-methyl-pyrazol-4-yl]pyridine-2-carboxylic acid

Using the General procedure for production of VHL ligand-based degraders via alkylation starting from the product of Preparation 4 (30 mg) and the product of Step B as the appropriate alkylating agent, 12 mg of the desired product was obtained. HRMS-ESI (m/z):[M+2H]²⁺ calcd for C₇₆H₁₀₃N₁₃O₇S₂: 686.8772. found: 686.8766.

### Example 3: 6-[3-(1,3-benzothiazol-2-ylamino)-4-methyl-6,7-dihydro-5H-pyrido[2,3-c]pyridazin-8-yl]-3-[1-[[3-[2-[[7-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-7-oxo-heptyl]-methyl-amino]ethoxy]-5,7-dimethyl-1-adamantyl]methyl]-5-methyl-pyrazol-4-yl]pyridine-2-carboxylic acid

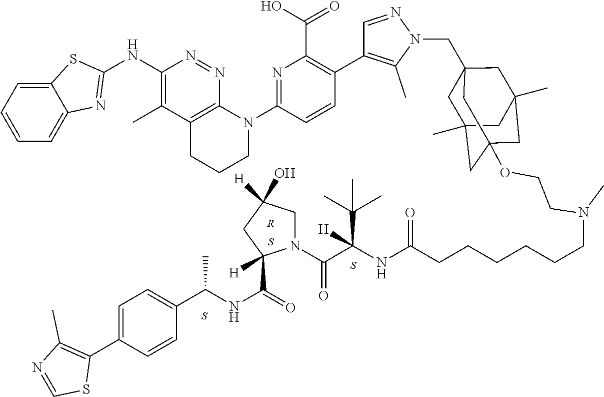

### Step A: (2S,4R)-1-[(2S)-2-(7-bromoheptanoylamino)-3,3-dimethyl-butanoyl]-4-hydroxy-N-[(1S)-1-[4-(4-methylthiazol-5-yl)phenyl]ethyl]pyrrolidine-2-carboxamide

Using General procedure for the acylation of VHL ligands starting from (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide, hydrogen chloride (1:1) (0.42 mmol) and 7-bromoheptanoic acid as the appropriate acid, 181 mg of the desired product was obtained. ¹H NMR (500 MHz, DMSO-d₆): δ ppm 8.98 (s, 1H), 8.38 (d, 1H), 7.81 (d, 1H), 7.43 (m, 2H), 7.37 (m, 2H), 5.10 (d, 1H), 4.91 (m, 1H), 4.52 (d, 1H), 4.41 (t, 1H), 4.25 (m, 1H), 3.62/3.58 (m+m, 2H), 3.51 (t, 2H), 2.45 (s, 3H), 2.24/2.11 (m+m, 2H), 2.00/1.78 (m+m, 2H), 1.77 (m, 2H), 1.56-1.19 (m, 6H), 1.37 (d, 3H), 0.93 (s, 9H); ¹³C NMR (125 MHz, DMSO-d₆) δ ppm 152.0, 129.3, 126.8, 69.2, 59.0, 56.8, 56.7, 48.2, 38.2, 35.6, 35.2, 32.6, 26.9, 22.9, 16.5; HRMS-ESI (m/z):[M+H]⁺ calcd for C₃₀H₄₄BrN₄O₄S: 635.2267. found: 635.2261.

### Step B: 6-[3-(1,3-benzothiazol-2-ylamino)-4-methyl-6,7-dihydro-5H-pyrido[2,3-c]pyridazin-8-yl]-3-[1-[[3-[2-[[7-[[(1S)-1-[(2S,4R)-4-hydroxy-2-[[(1S)-1-[4-(4-methylthiazol-5-yl)phenyl] ethyl]carbamoyl]pyrrolidine-1-carbonyl]-2,2-dimethyl-propyl]amino]-7-oxo-heptyl]-methyl-amino]ethoxy]-5,7-dimethyl-1-adamantyl]methyl]-5-methyl-pyrazol-4-yl]pyridine-2-carboxylic acid

Using the General procedure for production of VHL ligand-based degraders via alkylation starting from the product of Preparation 4 (30 mg) and the product of Step A as the appropriate alkylating agent, 10 mg of the desired product was obtained. HRMS-ESI (m/z):[M+2H]²⁺ calcd for C₇₁H₉₃N₁₃O₇S₂: 651.8381. found: 651.8376.

### Example 4: 6-[3-(1,3-benzothiazol-2-ylamino)-4-methyl-6,7-dihydro-5H-pyrido[2,3-c]pyridazin-8-yl]-3-[1-[[3-[2-[[14-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-14-oxo-tetradecyl]-methyl-amino]ethoxy]-5,7-dimethyl-1-adamantyl]methyl]-5-methyl-pyrazol-4-yl]pyridine-2-carboxylic acid

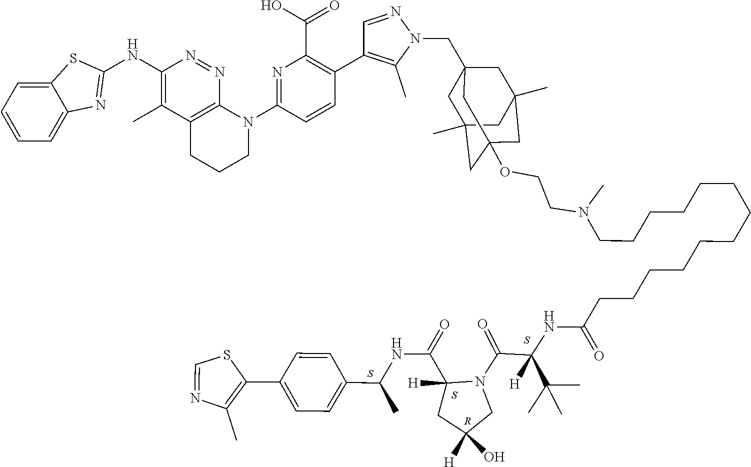

### Step A: (2S,4R)-4-hydroxy-1-[(2S)-2-(14-hydroxytetradecanoylamino)-3,3-dimethyl-butanoyl]-N-[(1S)-1-[4-(4-methylthiazol-5-yl)phenyl]ethyl]pyrrolidine-2-carboxamide

Using General procedure for the acylation of VHL ligands starting from (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide, hydrogen chloride (1:1) (0.42 mmol) and 14-hydroxytetradecanoic acid as the appropriate acid, 205 mg of the desired product was obtained. HRMS-ESI (m/z): [M+H]⁺ calcd for C₃₇H₅₉N₄O₅S: 671.4206. found: 671.4201.

### Step B: [14-[[(15)-1-[(2S,4R)-4-hydroxy-2-[[(15)-1-[4-(4-methylthiazol-5-yl)phenyl] ethyl]carbamoyl]pyrrolidine-1-carbonyl]-2,2-dimethyl-propyl]amino]-14-oxo-tetradecyl]4-methylbenzenesulfonate

Using the General procedure for the tosylation of the hydroxyalkyl VHL ligand-derivatives starting from the product of Step A (100 mg), 105 mg of the desired product was obtained. ¹H NMR (500 MHz, DMSO-d₆): δ ppm 8.99 (s, 1H), 8.38 (d, 1H), 7.79 (d, 1H), 7.78 (d, 2H), 7.48 (d, 2H), 7.43 (d, 2H), 7.38 (d, 2H), 5.08 (brs, 1H), 4.91 (qn, 1H), 4.51 (d, 1H), 4.41 (t, 1H), 4.27 (m, 1H), 3.99 (t, 2H), 3.61/3.58 (dd+dd, 2H), 2.45 (s, 3H), 2.42 (s, 3H), 2.24/2.09 (m+m, 2H), 2.00/1.78 (m+m, 2H), 1.53 (qn, 2H), 1.48/1.44 (m+m, 2H), 1.37 (d, 3H), 1.27-1.09 (m, 18H), 0.93 (s, 9H); 130 NMR (125 MHz, DMSO-d₆) δ ppm 152.0, 130.6, 129.3, 128.1, 126.8, 71.4, 69.2, 59.0, 56.8, 56.7, 48.2, 38.2, 35.3, 28.6, 26.9, 25.6, 22.9, 21.6, 16.4; HRMS-ESI (m/z): [M+H]⁺ calcd for C₄₄H₆₅N₄O₇S₂: 825.4295. found: 825.4285.

### Step C: 6-[3-(1,3-benzothiazol-2-ylamino)-4-methyl-6,7-dihydro-5H-pyrido[2,3-c]pyridazin-8-yl]-3-[1-[[3-[2-[[14-[[(1S)-1-[(2S,4R)-4-hydroxy-2-[[(1S)-1-[4-(4-methylthiazol-5-yl)phenyl] ethyl]carbamoyl]pyrrolidine-1-carbonyl]-2,2-dimethyl-propyl]amino]-14-oxo-tetradecyl]-methyl-amino]ethoxy]-5,7-dimethyl-1-adamantyl]methyl]-5-methyl-pyrazol-4-yl]pyridine-2-carboxylic acid

Using the General procedure for production of VHL ligand-based degraders via alkylation starting from the product of Preparation 4 (30 mg) and the product of Step B as the appropriate alkylating agent, 8 mg of the desired product was obtained. HRMS-ESI (m/z):[M+2H]²⁺ calcd for C₇₈H₁₀₇N₁₃O₇S₂: 700.8929. found: 700.8928.

### Example 5: 6-[3-(1,3-benzothiazol-2-ylamino)-4-methyl-6,7-dihydro-5H-pyrido[2,3-c]pyridazin-8-yl]-3-[1-[[3-[2-[[9-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-9-oxo-nonyl]-methyl-amino]ethoxy]-5,7-dimethyl-1-adamantyl] methyl]-5-methyl-pyrazol-4-yl]pyridine-2-carboxylic acid

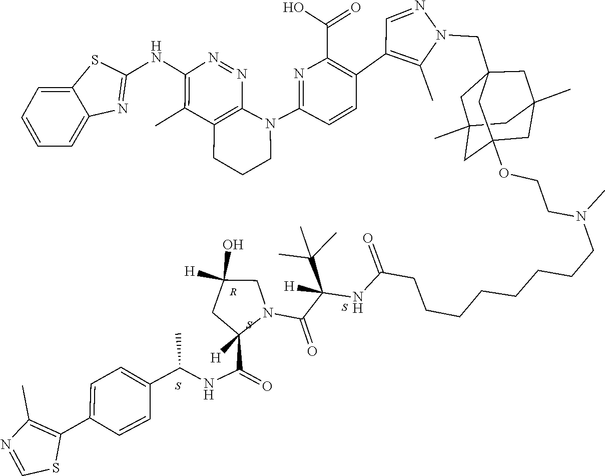

### Step A: (2S,4R)-1-[(2S)-2-(9-bromononanoylamino)-3,3-dimethyl-butanoyl]-4-hydroxy-N-[(1S)-1-[4-(4-methylthiazol-5-yl)phenyl]ethyl]pyrrolidine-2-carboxamide

Using General procedure for the acylation of VHL ligands starting from (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide, hydrogen chloride (1:1) (0.42 mmol) and 9-bromononanoic acid as the appropriate acid, 187 mg of the desired product was obtained. ¹H NMR (500 MHz, DMSO-d₆): δ ppm 8.99 (s, 1H), 8.38 (d, 1H), 7.79 (d, 1H), 7.44 (m, 2H), 7.38 (m, 2H), 5.10 (d, 1H), 4.91 (m, 1H), 4.51 (d, 1H), 4.41 (t, 1H), 4.27 (m, 1H), 3.61/3.58 (m+m, 2H), 3.52 (t, 2H), 2.45 (s, 3H), 2.24/2.11 (m+m, 2H), 2/1.78 (m+m, 2H), 1.78 (m, 2H), 1.49/1.43 (m+m, 2H), 1.40-1.19 (m, 8H), 1.37 (d, 3H), 0.93 (s, 9H); ¹³C NMR (125 MHz, DMSO-d₆) δ ppm 152.0, 129.3, 126.8, 69.2, 59.0, 56.8, 56.7, 48.1, 38.2, 35.7, 35.3, 32.7, 26.9, 25.8, 22.9, 16.5; HRMS-ESI (m/z): [M+H]⁺ calcd for C₃₂H₄₈BrN₄O₄S: 663.2580. found: 663.2571.

### Step B: 6-[3-(1,3-benzothiazol-2-ylamino)-4-methyl-6,7-dihydro-5H-pyrido[2,3-c]pyridazin-8-yl]-3-[1-[[3-[2-[[9-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-9-oxo-nonyl]-methyl-amino]ethoxy]-5,7-dimethyl-1-adamantyl]methyl]-5-methyl-pyrazol-4-yl]pyridine-2-carboxylic acid

Using the General procedure for production of VHL ligand-based degraders via alkylation starting from the product of Preparation 4 (30 mg) and the product of Step A as the appropriate alkylating agent, 18 mg of the desired product was obtained. HRMS-ESI (m/z):[M+2H]²⁺ calcd for C₇₃H₉₇N₁₃O₇S₂: 655.8538. found: 665.8533.

### Example 6: 6-[3-(1,3-benzothiazol-2-ylamino)-4-methyl-6,7-dihydro-5H-pyrido[2,3-c]pyridazin-8-yl]-3-[1-[[3-[2-[[8-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-8-oxo-octyl]-methyl-amino]ethoxy]-5,7-dimethyl-1-adamantyl]methyl]-5-methyl-pyrazol-4-yl]pyridine-2-carboxylic acid

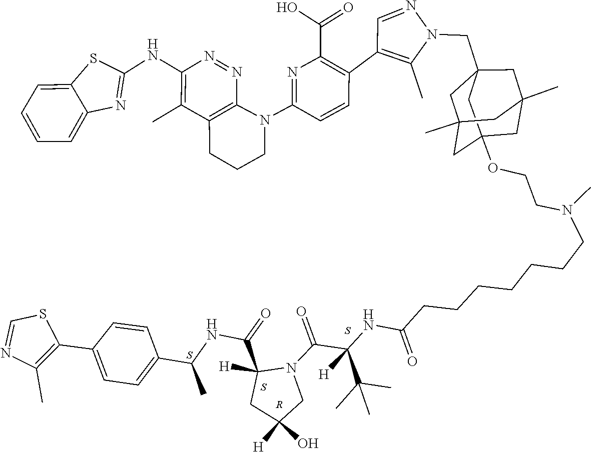

### Step A: (2S,4R)-1-[(2S)-2-(8-bromooctanoylamino)-3,3-dimethyl-butanoyl]-4-hydroxy-N-[(1S)-1-[4-(4-methylthiazol-5-yl)phenyl]ethyl]pyrrolidine-2-carboxamide

Using General procedure for the acylation of VHL ligands starting from (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide, hydrogen chloride (1:1) (0.42 mmol) and 8-bromooctanoic acid as the appropriate acid, 194 mg of the desired product was obtained. 1H NMR (500 MHz, DMSO-d₆) δ ppm 8.99 (s, 1H), 8.38 (d, 1H), 7.80 (d, 1H), 7.43 (m, 2H), 7.38 (m, 2H), 5.10 (d, 1H), 4.91 (m, 1H), 4.52 (d, 1H), 4.41 (t, 1H), 4.28 (m, 1H), 3.61/3.58 (m+m, 2H), 3.52 (t, 2H), 2.45 (s, 3H), 2.25/2.10 (m+m, 2H), 2.01/1.78 (m+m, 2H), 1.78 (m, 2H), 1.58-1.17 (m, 8H), 1.37 (d, 3H), 0.93 (s, 9H); ¹³C NMR (125 MHz, DMSO-d₆) δ ppm 152.0, 129.3, 126.8, 69.2, 59.0, 56.8, 56.7, 48.2, 38.2, 35.7, 35.3, 32.7, 26.9, 22.9, 16.5.

### Step B: 6-[3-(1,3-benzothiazol-2-ylamino)-4-methyl-6,7-dihydro-5H-pyrido[2,3-c]pyridazin-8-yl]-3-[1-[[3-[2-[[8-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-8-oxo-octyl]-methyl-amino]ethoxy]-5,7-dimethyl-1-adamantyl]methyl]-5-methyl-pyrazol-4-yl]pyridine-2-carboxylic acid

Using the General procedure for production of VHL ligand-based degraders via alkylation starting from the product of Preparation 4 (30 mg) and the product of Step A as the appropriate alkylating agent, 20 mg of the desired product was obtained. HRMS-ESI (m/z): [M+2H]²⁺ calcd for C₇₂H₉₅N₁₃O₇S₂: 658.8460. found: 658.8460.

### Example 7: 6-[3-(1,3-benzothiazol-2-ylamino)-4-methyl-6,7-dihydro-5H-pyrido[2,3-c]pyridazin-8-yl]-3-[1-[[3-[2-[[15-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-15-oxo-pentadecyl]-methyl-amino]ethoxy]-5,7-dimethyl-1-adamantyl]methyl]-5-methyl-pyrazol-4-yl]pyridine-2-carboxylic acid

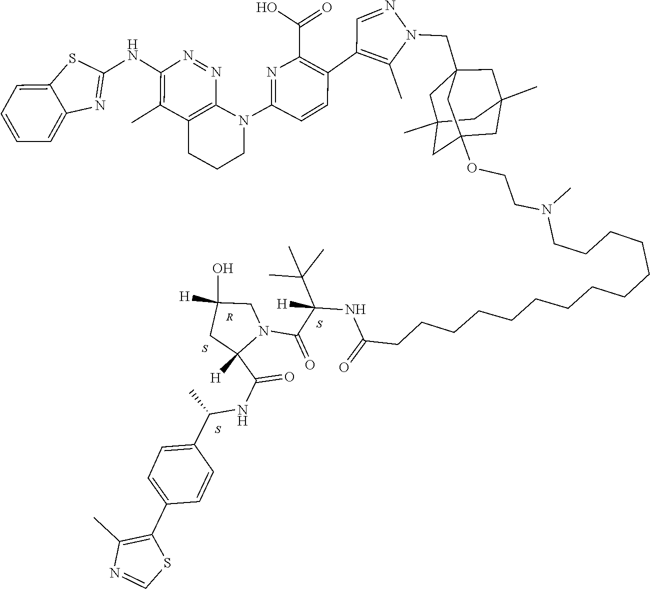

### Step A: (2S,4R)-4-hydroxy-1-[(2S)-2-(15-hydroxypentadecanoylamino)-3,3-dimethyl-butanoyl]-N-[(1S)-1-[4-(4-methylthiazol-5-yl)phenyl]ethyl]pyrrolidine-2-carboxamide

Using General procedure for the acylation of VHL ligands starting from (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide, hydrogen chloride (1:1) (0.42 mmol) and 15-hydroxypentadecanoic acid as the appropriate acid, 154 mg of the desired product was obtained. ¹H NMR (500 MHz, DMSO-d₆) δ ppm 8.98 (s, 1H), 8.38 (d, 1H), 7.79 (d, 1H), 7.43 (d, 2H), 7.37 (d, 2H), 5.10 (d, 1H), 4.91 (m, 1H), 4.51 (d, 1H), 4.41 (t, 1H), 4.32 (t, 1H), 4.27 (m, 1H), 3.64-3.55 (m, 2H), 3.36 (q, 2H), 2.45 (s, 3H), 2.24/2.09 (m+m, 2H), 2/1.78 (m+m, 2H), 1.52-1.38 (m, 2H), 1.38 (q, 2H), 1.37 (d, 3H), 1.29-1.19 (m, 20H), 0.93 (s, 9H); ¹³C NMR (125 MHz, DMSO-d₆) δ ppm 172.5, 171.1, 170.1, 152.0, 129.3, 126.8, 69.2, 61.2, 59.0, 56.8, 56.7, 48.2, 38.2, 35.3, 33.0, 26.9, 26.0, 22.9, 16.5; HRMS-ESI (m/z): [M+H]⁺ calcd for C₃₈H₆₁N₄O₅S: 685.4363. found: 685.4352.

### Step B: [15-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-15-oxo-pentadecyl]4-methylbenzenesulfonate

Using the General procedure for the tosylation of the hydroxyalkyl VHL ligand-derivatives starting from the product of Step A (150 mg), 53 mg of the desired product was obtained. ¹H NMR (500 MHz, DMSO-d₆) δ ppm 8.98 (s, 1H), 8.38 (d, 1H), 7.79 (d, 1H), 7.78 (dm, 2H), 7.48 (dm, 2H), 7.43 (d, 2H), 7.37 (d, 2H), 5.10 (d, 1H), 4.91 (m, 1H), 4.51 (d, 1H), 4.41 (t, 1H), 4.27 (m, 1H), 3.99 (t, 2H), 3.64-3.55 (m, 2H), 2.45 (s, 3H), 2.42 (s, 3H), 2.24/2.09 (m+m, 2H), 2.00/1.78 (m+m, 2H), 1.53 (m, 2H), 1.52-1.38 (m, 2H), 1.37 (d, 3H), 1.30-1.10 (br., 20H), 0.93 (s, 9H); ¹³C NMR (125 MHz, DMSO-d₆) δ ppm 172.5, 171.1, 170.1, 152.0, 130.6, 129.3, 128.0, 126.8, 71.4, 69.2, 59.0, 56.8, 56.7, 48.2, 38.2, 35.3, 28.6, 26.9, 26.0, 22.9, 21.6, 16.5; HRMS-ESI (m/z): [M+H]⁺ calcd for C₄₅H₆₇N₄O₇S₂: 839.4451. found: 839.4447.

### Step C: 6-[3-(1,3-benzothiazol-2-ylamino)-4-methyl-6,7-dihydro-5H-pyrido[2,3-c]pyridazin-8-yl]-3-[1-[[3-[2-[[15-[[(1S)-1-[(2S,4R)-4-hydroxy-2-[[(1S)-1-[4-(4-methylthiazol-5-yl)phenyl] ethyl]carbamoyl]pyrrolidine-1-carbonyl]-2,2-dimethyl-propyl]amino]-15-oxo-pentadecyl]-methyl-amino]ethoxy]-5,7-dimethyl-1-adamantyl]methyl]-5-methyl-pyrazol-4-yl]pyridine-2-carboxylic acid

Using the General procedure for production of VHL ligand-based degraders via alkylation starting from the product of Preparation 4 (30 mg) and the product of Step B as the appropriate alkylating agent, 18 mg of the desired product was obtained. HRMS-ESI (m/z): [M+2H]²⁺ calcd for C₇₉H₁₀₉N₁₃O₇S₂: 707.9007. found: 707.9002.

### Example 8: 6-[3-(1,3-benzothiazol-2-ylamino)-4-methyl-6,7-dihydro-5H-pyrido[2,3-c]pyridazin-8-yl]-3-[1-[[3-[2-[[6-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-6-oxo-hexyl]-methyl-amino]ethoxy]-5,7-dimethyl-1-adamantyl]methyl]-5-methyl-pyrazol-4-yl]pyridine-2-carboxylic acid

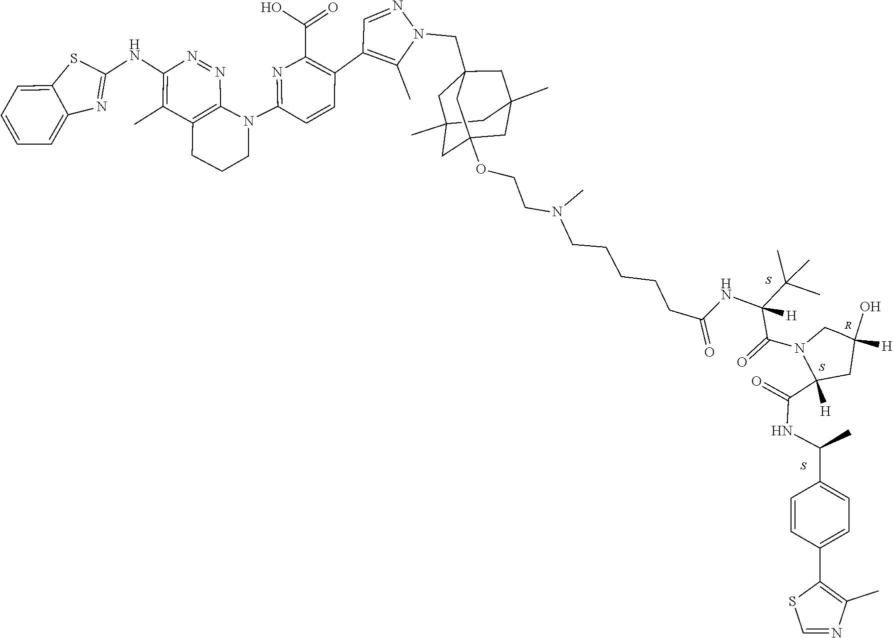

### Step A: (2S,4R)-1-[(2S)-2-(6-bromohexanoylamino)-3,3-dimethyl-butanoyl]-4-hydroxy-N-[(1S)-1-[4-(4-methylthiazol-5-yl)phenyl]ethyl]pyrrolidine-2-carboxamide

Using General procedure for the acylation of VHL ligands starting from (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide, hydrogen chloride (1:1) (0.42 mmol) and 6-bromohexanoic acid as the appropriate acid, 156 mg of the desired product was obtained. ¹H NMR (500 MHz, DMSO-d₆) δ ppm 8.98 (s, 1H), 8.38 (d, 1H), 7.84 (d, 1H), 7.44 (dm, 2H), 7.38 (dm, 2H), 5.10 (br., 1H), 4.91 (m, 1H), 4.52 (d, 1H), 4.41 (t, 1H), 4.27 (br., 1H), 3.64-3.55 (m, 2H), 3.51 (t, 2H), 2.45 (s, 3H), 2.26/2.13 (t, 2H), 2.00/1.78 (m+m, 2H), 1.79 (m, 2H), 1.58-1.42 (m, 2H), 1.41-1.29 (m, 2H), 1.37 (d, 3H), 0.93 (s, 9H); ¹³C NMR (125 MHz, DMSO-d₆) δ ppm 172.3, 171.0, 170.1, 152.0, 129.3, 126.8, 69.2, 59.0, 56.8, 56.7, 48.2, 38.2, 35.6, 35.1, 32.4, 27.7, 26.9, 25.0, 22.9, 16.5; HRMS-ESI (m/z): [M+H]⁺ calcd for C₂₉H₄₂BrN₄O₄S: 621.2110. found: 621.2099.

### Step B: 6-[3-(1,3-benzothiazol-2-ylamino)-4-methyl-6,7-dihydro-5H-pyrido[2,3-c]pyridazin-8-yl]-3-[1-[[3-[2-[[6-[[(1S)-1-[(2S,4R)-4-hydroxy-2-[[(1S)-1-[4-(4-methylthiazol-5-yl)phenyl] ethyl]carbamoyl]pyrrolidine-1-carbonyl]-2,2-dimethyl-propyl]amino]-6-oxo-hexyl]-methyl-amino]ethoxy]-5,7-dimethyl-1-adamantyl]methyl]-5-methyl-pyrazol-4-yl]pyridine-2-carboxylic acid

Using the General procedure for production of VHL ligand-based degraders via alkylation starting from the product of Preparation 4 (30 mg) and the product of Step A as the appropriate alkylating agent, 15 mg of the desired product was obtained. HRMS-ESI (m/z): [M+2H]²⁺ calcd for C₇₀H₉₁N₁₃O₇S₂: 644.8329. found: 644.8297.

### Example 9: 6-[3-(1,3-benzothiazol-2-ylamino)-4-methyl-6,7-dihydro-5H-pyrido[2,3-c]pyridazin-8-yl]-3-[1-[[3-[2-[2-[2-[[2-(2,6-dioxo-3-piperidyl)-1,3-dioxo-isoindolin-4-yl]amino] ethoxy]ethyl-methyl-amino]ethoxy]-5,7-dimethyl-1-adamantyl]methyl]-5-methyl-pyrazol-4-yl]pyridine-2-carboxylic acid

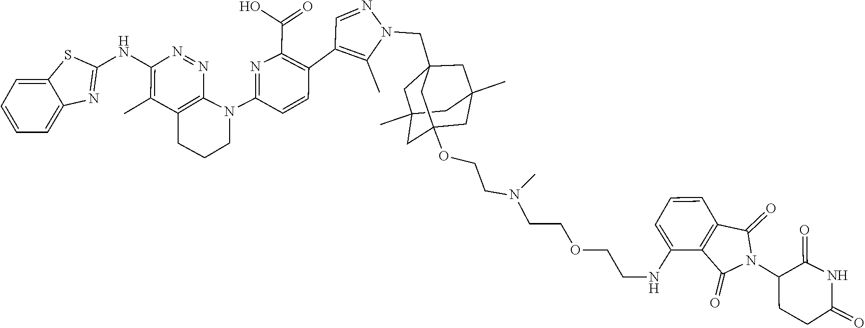 [SMILES: Cc1c(Nc2nc3ccccc3s2)nnc2c1CCCN2c1ccc(-c2cnn(CC34CC5(C)CC(C)(C3)CC(OCCN(C)CCOCCNc3cccc6c3C(=O)N(C3CCC(=O)NC3=O)C6=O)(C5)C4)c2C)c(C(=O)O)n1]

### Step A: 2-(2,6-dioxo-3-piperidyl)-4-[2-(2-hydroxyethoxy)ethylamino]isoindoline-1,3-dione

Using General procedure for the nucleophilic substitution of fluoro-thalidomide starting from 2-(2,6-dioxo-3-piperidyl)-4-fluoro-isoindoline-1,3-dione (0.72 mmol) and 2-(2-aminoethoxy) ethanol as the appropriate amine, 62 mg of the desired product was obtained. ¹H NMR (500 MHz, DMSO-d₆) δ ppm 11.1 (s, 1H), 7.59 (dd, 1H), 7.15 (d, 1H), 7.04 (d, 1H), 6.61 (t, 1H), 5.06 (dd, 1H), 4.58 (br., 1H), 3.62 (t, 2H), 3.51 (m, 2H), 3.49-3.44 (m, 2H), 3.49-3.44 (m, 2H), 2.88/2.59 (ddd+dm, 2H), 2.50/2.02 (m+m, 2H); ¹³C NMR (125 MHz, DMSO-d₆) δ ppm 173.3/170.6, 169.4/167.8, 136.7, 117.9, 111.1, 72.7, 69.3, 60.7, 49.0, 42.2, 31.4, 22.6; HRMS-ESI (m/z): [M+H]⁺ calcd for C₁₇H₂₀N₃O₆: 362.1352. found: 362.1350.

### Step B: 2-(2,6-dioxo-3-piperidyl)-4-[2-(2-iodoethoxy)ethylamino]isoindoline-1,3-dione

Using the General procedure for the iodination of hydroxyalkyl derivative of thalidomide starting from the product of Step A (60 mg), 55 mg of the desired product was obtained. ¹H NMR (500 MHz, DMSO-d₆) δ ppm 11.1 (s, 1H), 7.59 (dd, 1H), 7.17 (d, 1H), 7.04 (d, 1H), 6.62 (t, 1H), 5.06 (dd, 1H), 3.70 (t, 2H), 3.65 (t, 2H), 3.49 (q, 2H), 3.33 (t, 2H), 2.88/2.59 (ddd+dm, 2H), 2.50/2.02 (m+m, 2H); ¹³C NMR (125 MHz, DMSO-d₆) δ ppm 173.3/170.6, 169.4/167.8, 136.7, 118.0, 111.2, 71.2, 68.8, 49.0, 42.2, 31.4, 22.6, 5.7; HRMS-ESI (m/z): [M+H]⁺ calcd for C₁₇H₁₉IN₃O₅: 472.0369. found: 472.0360.

### Step C: 6-[3-(1,3-benzothiazol-2-ylamino)-4-methyl-6,7-dihydro-5H-pyrido[2,3-c]pyridazin-8-yl]-3-[1-[[3-[2-[2-[2-[[2-(2,6-dioxo-3-piperidyl)-1,3-dioxo-isoindolin-4-yl]amino]ethoxy] ethyl-methyl-amino]ethoxy]-5,7-dimethyl-1-adamantyl]methyl]-5-methyl-pyrazol-4-yl]pyridine-2-carboxylic acid

Using the General procedure for the production of thalidomide-based degraders via alkylation starting from the product of Preparation 4 (30 mg) and the product of Step B as the appropriate alkylating agent, 27 mg of the desired product was obtained. HRMS-ESI (m/z): [M+2H]²⁺ calcd for C₅₈H₆₈N₁₂O₈S: 546.2502. found: 546.2506.

### Example 10: 6-[3-(1,3-benzothiazol-2-ylamino)-4-methyl-6,7-dihydro-5H-pyrido[2,3-c]pyridazin-8-yl]-3-[1-[[3-[2-[8-[2-(2,6-dioxo-3-piperidyl)-1,3-dioxo-isoindolin-5-yl]oxyoctyl-methyl-amino]ethoxy]-5,7-dimethyl-1-adamantyl]methyl]-5-methyl-pyrazol-4-yl]pyridine-2-carboxylic acid

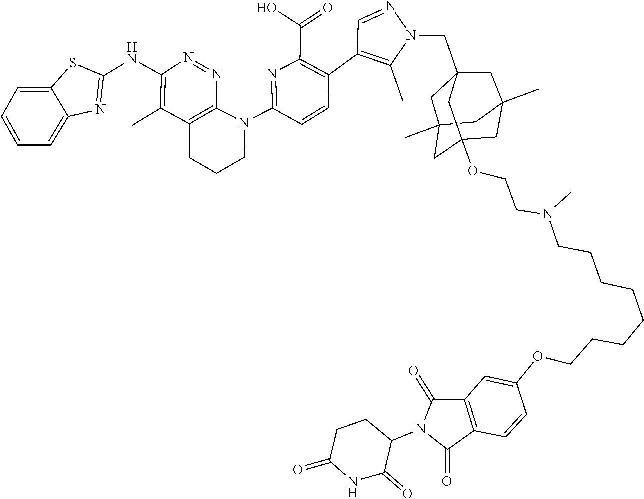 [SMILES: Cc1c(Nc2nc3ccccc3s2)nnc2c1CCCN2c1ccc(-c2cnn(CC34CC5(C)CC(C)(C3)CC(OCCN(C)CCCCCCCCOc3ccc6c(c3)C(=O)N(C3CCC(=O)NC3=O)C6=O)(C5)C4)c2C)c(C(=O)O)n1]

### Step A: 5-(8-bromooctoxy)-2-(2,6-dioxo-3-piperidyl)isoindoline-1,3-dione

Using General procedure for the alkylation of the hydroxy-thalidomide starting from 2-(2,6-dioxo-3-piperidyl)-5-hydroxy-isoindoline-1,3-dione (0.44 mmol) and 1,8-dibromooctane as the appropriate alkylating agent, 85 mg of the desired product was obtained. 1H NMR (500 MHz, DMSO-d₆) δ ppm 11.12 (s, 1H), 7.82 (d, 1H), 7.42 (d, 1H), 7.34 (dd, 1H), 5.12 (dd, 1H), 4.18 (t, 2H), 3.53 (t, 2H), 2.89/2.59 (m+brd., 2H), 2.53/2.04 (m+m, 2H), 1.84-1.70 (m, 4H), 1.48-1.26 (m, 8H); ¹³C NMR (125 MHz, DMSO-d₆) δ ppm 173.3/170.4, 167.4/167.3, 125.8, 121.2, 109.3, 69.2, 49.4, 35.7, 31.4, 22.5; HRMS-ESI (m/z): [M+H]⁺ calcd for C₂₁H₂₆BrN₂O₅: 465.1025. found: 465.1017.

### Step B: 6-[3-(1,3-benzothiazol-2-ylamino)-4-methyl-6,7-dihydro-5H-pyrido[2,3-c]pyridazin-8-yl]-3-[1-[[3-[2-[8-[2-(2,6-dioxo-3-piperidyl)-1,3-dioxo-isoindolin-5-yl]oxyoctyl-methyl-amino]ethoxy]-5,7-dimethyl-1-adamantyl]methyl]-5-methyl-pyrazol-4-yl]pyridine-2-carboxylic acid

Using the General procedure for the production of thalidomide-based degraders via alkylation starting from the product of Preparation 4 (20 mg) and the product of Step A as the appropriate alkylating agent, 17 mg of the desired product was obtained. HRMS-ESI (m/z): [M+H]⁺ calcd for C₆₂H₇₄N₁₁O₈S: 1132.5443. found: 1132.5432.

### Example 11: 6-[3-(1,3-benzothiazol-2-ylamino)-4-methyl-6,7-dihydro-5H-pyrido[2,3-c]pyridazin-8-yl]-3-[1-[[3-[2-[2-[2-[2-[2-[[2-(2,6-dioxo-3-piperidyl)-1,3-dioxo-isoindolin-4-yl]amino]ethoxy]ethoxy]ethoxy]ethyl-methyl-amino]ethoxy]-5,7-dimethyl-1-adamantyl]methyl]-5-methyl-pyrazol-4-yl]pyridine-2-carboxylic acid

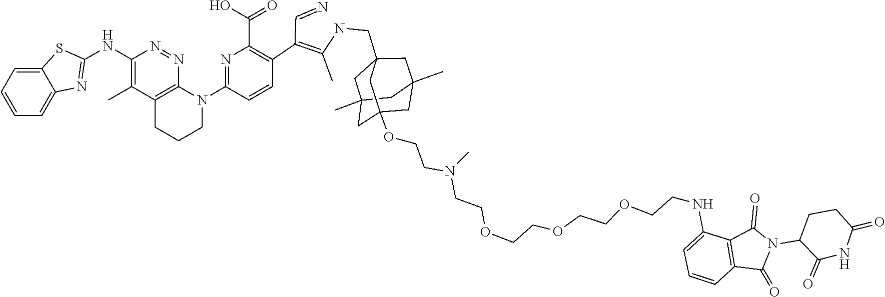

### Step A: 2-(2,6-dioxo-3-piperidyl)-4-[2-[2-[2-(2-hydroxyethoxy)ethoxy]ethoxy]ethylamino] isoindoline-1,3-dione

Using General procedure for the nucleophilic substitution of fluoro-thalidomide starting from 2-(2,6-dioxo-3-piperidyl)-4-fluoro-isoindoline-1,3-dione (0.72 mmol) and 2-[2-[2-(2-amino ethoxy)ethoxy]ethoxy]ethanol as the appropriate amine, 169 mg of the desired product was obtained. 1H NMR (500 MHz, DMSO-d₆) δ ppm 11.10 (s, 1H), 7.58 (dd, 1H), 7.15 (d, 1H), 7.04 (d, 1H), 6.61 (t, 1H), 6.46 (t, 2H), 5.05 (dd, 1H), 4.54 (brs, 1H), 3.62 (t, 2H), 3.6-3.4 (m, 8H), 3.47 (m, 2H), 3.39 (t, 2H), 2.88/2.58 (m+m, 2H), 2.53/2.02 (m+m, 2H); ¹³C NMR (125 MHz, DMSO-d₆) δ ppm 136.7, 117.9, 111.1, 72.8, 69.3, 60.7, 49.0, 42.1, 31.4, 22.6; HRMS-ESI (m/z): [M+H]⁺ calcd for C₂₁H₂₈N₃O₈: 450.1876. found: 450.1871.

### Step B: 2-(2,6-dioxo-3-piperidyl)-4-[2-[2-[2-(2-iodoethoxy)ethoxy]ethoxy] ethylamino]isoindoline-1,3-dione

Using the General procedure for the iodination of hydroxyalkyl derivative of thalidomide starting from the product of Step A (117 mg), 90 mg of the desired product was obtained. HRMS-ESI (m/z): [M+H]⁺ calcd for C₂₁H₂₇IN₃O₇: 560.0894. found: 560.0895.

### Step C: 6-[3-(1,3-benzothiazol-2-ylamino)-4-methyl-6,7-dihydro-5H-pyrido[2,3-c]pyridazin-8-yl]-3-[1-[[3-[2-[2-[2-[2-[2-[[2-(2,6-dioxo-3-piperidyl)-1,3-dioxo-isoindolin-4-yl]amino] ethoxy]ethoxy]ethoxy]ethyl-methyl-amino]ethoxy]-5,7-dimethyl-1-adamantyl]methyl]-5-methyl-pyrazol-4-yl]pyridine-2-carboxylic acid

Using the General procedure for the production of thalidomide-based degraders via alkylation starting from the product of Preparation 4 (40 mg) and the product of Step B as the appropriate alkylating agent, 7 mg of the desired product was obtained. HRMS-ESI (m/z): [M+2H]²⁺ calcd for C₆₂H₇₆N₁₂O₁₀S: 590.2764. found: 590.2758.

### Example 12: 6-[3-(1,3-benzothiazol-2-ylamino)-4-methyl-6,7-dihydro-5H-pyrido[2,3-c]pyridazin-8-yl]-3-[1-[[3-[2-[2-[2-[2-[[2-(2,6-dioxo-3-piperidyl)-1,3-dioxo-isoindolin-4-yl]amino]ethoxy]ethoxy]ethyl-methyl-amino]ethoxy]-5,7-dimethyl-1-adamantyl] methyl]-5-methyl-pyrazol-4-yl]pyridine-2-carboxylic acid

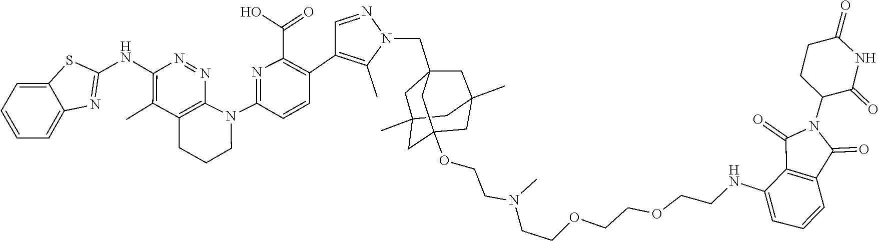 [SMILES: Cc1c(Nc2nc3ccccc3s2)nnc2c1CCCN2c1ccc(-c2cnn(CC34CC5(C)CC(C)(C3)CC(OCCN(C)CCOCCOCCNc3cccc6c3C(=O)N(C3CCC(=O)NC3=O)C6=O)(C5)C4)c2C)c(C(=O)O)n1]

### Step A: 2-(2,6-dioxo-3-piperidyl)-4-[2-[2-(2-hydroxyethoxy)ethoxy]ethylamino]isoindoline-1,3-dione

Using General procedure for the nucleophilic substitution of fluoro-thalidomide starting from 2-(2,6-dioxo-3-piperidyl)-4-fluoro-isoindoline-1,3-dione (0.72 mmol) and 2-[2-(2-amino ethoxy)ethoxy]ethanol as the appropriate amine, 40 mg of the desired product was obtained. ¹H NMR (500 MHz, DMSO-d₆) δ ppm 11.1 (s, 1H), 7.58 (dd, 1H), 7.15 (d, 1H), 7.04 (d, 1H), 6.61 (t, 1H), 5.05 (dd, 1H), 3.65-3.38 (m, 10H), 3.47 (m, 2H), 2.88/2.58 (ddd+dm, 2H), 2.52/2.02 (m+m, 2H); ¹³C NMR (125 MHz, DMSO-d₆) δ ppm 173.3/170.6, 169.4/167.8, 136.7, 117.9, 111.1, 49.0, 42.2, 31.4, 22.6; HRMS-ESI (m/z): [M+H]⁺ calcd for C₁₉H₂₄N₃O₇: 406.1614. found: 406.1608.

### Step B: 2-(2,6-dioxo-3-piperidyl)-4-[2-[2-(2-iodoethoxy)ethoxy]ethylamino] isoindoline-1,3-dione

Using the General procedure for the iodination of hydroxyalkyl derivative of thalidomide starting from the product of Step A (40 mg), 34 mg of the desired product was obtained. ¹H NMR (500 MHz, DMSO-d₆) δ ppm 11.10 (s, 1H), 7.58 (dd, 1H), 7.15 (d, 1H), 7.04 (d, 1H), 6.61 (t, 1H), 5.05 (dd, 1H), 3.68-3.55 (m, 8H), 3.47 (q, 2H), 3.29 (t, 2H), 2.88/2.58 (ddd+dm, 2H), 2.5/2.02 (m+m, 2H), ¹³C NMR (125 MHz, DMSO-d₆) δ ppm 136.5, 117.9, 111.1, 49.0, 42.1, 31.3, 22.6, 5.7; HRMS-ESI (m/z): [M+H]⁺ calcd for C₉H₂₃IN₃O₆: 516.0632. found: 516.0626.0.

### Step C: 6-[3-(1,3-benzothiazol-2-ylamino)-4-methyl-6,7-dihydro-5H-pyrido[2,3-c]pyridazin-8-yl]-3-[1-[[3-[2-[2-[2-[2-[[2-(2,6-dioxo-3-piperidyl)-1,3-dioxo-isoindolin-4-yl]amino]ethoxy] ethoxy]ethyl-methyl-amino]ethoxy]-5,7-dimethyl-1-adamantyl]methyl]-5-methyl-pyrazol-4-yl]pyridine-2-carboxylic acid

Using the General procedure for the production of thalidomide-based degraders via alkylation starting from the product of Preparation 4 (50 mg) and the product of Step B as the appropriate alkylating agent, 12 mg of the desired product was obtained. HRMS-ESI (m/z): [M+2H]²⁺ calcd for C₆₀H₇₂N₁₂O₉S: 568.2633. found: 568.2628.

### Example 13: 6-[3-(1,3-benzothiazol-2-ylamino)-4-methyl-6,7-dihydro-5H-pyrido[2,3-c]pyridazin-8-yl]-3-[1-[[3-[2-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]ethyl-methyl-amino]ethoxy]-5,7-dimethyl-1-adamantyl]methyl]-5-methyl-pyrazol-4-yl]pyridine-2-carboxylic acid

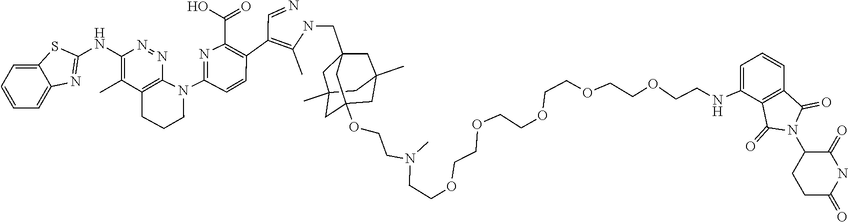

### Step A: 2-(2,6-dioxo-3-piperidyl)-4-[2-[2-[2-[2-[2-(2-hydroxyethoxy)ethoxy]ethoxy]ethoxy]ethoxy]ethylamino]isoindoline-1,3-dione

Using General procedure for the nucleophilic substitution of fluoro-thalidomide starting from 2-(2,6-dioxo-3-piperidyl)-4-fluoro-isoindoline-1,3-dione (1.45 mmol) and 2-[2-[2-[2-[2-(2-aminoethoxy)ethoxy]ethoxy]ethoxy]ethoxy]ethanol as the appropriate amine, 500 mg of the desired product was obtained. HRMS-ESI (m/z): [M+H]⁺ calcd for C₂₅H₃₆N₃O₁₀: 538.2401. found: 538.2396.

### Step B: 2-(2,6-dioxo-3-piperidyl)-4-[2-[2-[2-[2-[2-(2-iodoethoxy)ethoxy]ethoxy]ethoxy]ethoxy]ethylamino]isoindoline-1,3-dione

Using the General procedure for the iodination of hydroxyalkyl derivative of thalidomide starting from the product of Step A (500 mg), 272 mg of the desired product was obtained. ¹H NMR (500 MHz, DMSO-d₆) δ ppm 11.10 (s, 1H), 7.59 (dd, 1H), 7.15 (d, 1H), 7.04 (d, 1H), 6.61 (t, 1H), 5.06 (dd, 1H), 3.65 (t, 2H), 3.62 (t, 2H), 3.58-3.48 (m, 16H), 3.47 (q, 2H), 3.31 (t, 2H), 2.88/2.58 (td+dd, 2H), 2.50/2.02 (dd+dt, 2H); ¹³C NMR (125 MHz, DMSO-d₆) δ ppm 173.3, 170.6, 169.4, 167.8, 146.9, 136.7, 132.6, 118.0, 111.1, 109.7, 71.4, 69.3, 49.0, 42.2, 31.4, 22.6, 6.0; HRMS-ESI (m/z): [M+H]⁺ calcd for C₂₅H₃₅IN₃O₉: 648.1418. found: 648.1411.

### Step C: 6-[3-(1,3-benzothiazol-2-ylamino)-4-methyl-6,7-dihydro-5H-pyrido[2,3-c]pyridazin-8-yl]-3-[1-[[3-[2-[2-[2-[2-[2-[2-[2-[[2-(2,6-dioxo-3-piperidyl)-1,3-dioxo-isoindolin-4-yl]amino] ethoxy]ethoxy]ethoxy]ethoxy]ethoxy]ethyl-methyl-amino]ethoxy]-5,7-dimethyl-1-adamantyl]methyl]-5-methyl-pyrazol-4-yl]pyridine-2-carboxylic acid

Using the General procedure for the production of thalidomide-based degraders via alkylation starting from the product of Preparation 4 (30 mg) and the product of Step B as the appropriate alkylating agent, 22 mg of the desired product was obtained. HRMS-ESI (m/z): [M+2H]²⁺ calcd for C₆₆H₈₄N₁₂O₁₂S: 634.3026. found: 634.3019.

### Example 14: 6-[3-(1,3-benzothiazol-2-ylamino)-4-methyl-6,7-dihydro-5H-pyrido[2,3-c]pyridazin-8-yl]-3-[1-[[3-[2-[6-[2-(2,6-dioxo-3-piperidyl)-1,3-dioxo-isoindolin-5-yl]oxy hexyl-methyl-amino]ethoxy]-5,7-dimethyl-1-adamantyl]methyl]-5-methyl-pyrazol-4-yl]pyridine-2-carboxylic acid

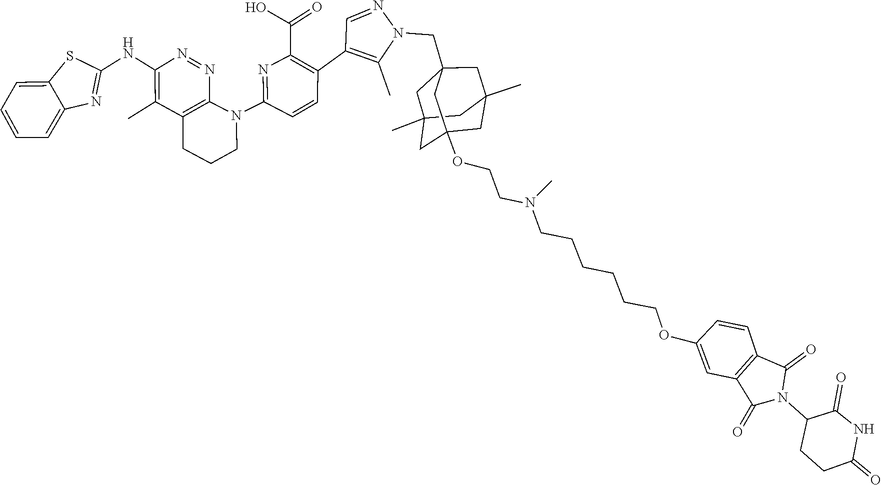 [SMILES: Cc1c(Nc2nc3ccccc3s2)nnc2c1CCCN2c1ccc(-c2cnn(CC34CC5(C)CC(C)(C3)CC(OCCN(C)CCCCCCOc3ccc6c(c3)C(=O)N(C3CCC(=O)NC3=O)C6=O)(C5)C4)c2C)c(C(=O)O)n1]

### Step A: 5-(6-bromohexoxy)-2-(2,6-dioxo-3-piperidyl)isoindoline-1,3-dione

Using General procedure for the alkylation of the hydroxy-thalidomide starting from 2-(2,6-dioxo-3-piperidyl)-5-hydroxy-isoindoline-1,3-dione (0.44 mmol) and 1,6-dibromohexane as the appropriate alkylating agent, 108 mg of the desired product was obtained. ¹H NMR (500 MHz, DMSO-d₆) δ ppm 11.12 (s, 1H), 7.83 (d, 1H), 7.43 (d, 1H), 7.35 (dd, 1H), 5.12 (dd, 1H), 4.17 (t, 2H), 3.54 (t, 2H), 2.89/2.59 (td+dd, 2H), 2.52/2.05 (dd+dt, 2H), 1.83 (qn, 2H), 1.76 (qn, 2H), 1.46 (qn, 2H), 1.45 (qn, 2H); ¹³C NMR (125 MHz, DMSO-d₆) δ ppm 173.3, 170.4, 167.4, 167.3, 164.6, 134.4, 125.8, 123.4, 121.2, 109.3, 69.1, 49.4, 35.6, 32.6, 31.4, 28.7, 27.7, 25, 22.5; HRMS-ESI (m/z): [M+H]⁺ calcd for C₁₉H₂₂BrN₂O₅: 437.0712. found: 437.0714.

### Step B: 6-[3-(1,3-benzothiazol-2-ylamino)-4-methyl-6,7-dihydro-5H-pyrido[2,3-c]pyridazin-8-yl]-3-[1-[[3-[2-[6-[2-(2,6-dioxo-3-piperidyl)-1,3-dioxo-isoindolin-5-yl]oxohexyl-methyl-amino]ethoxy]-5,7-dimethyl-1-adamantyl]methyl]-5-methyl-pyrazol-4-yl]pyridine-2-carboxylic acid

Using the General procedure for the production of thalidomide-based degraders via alkylation starting from the product of Preparation 4 (15 mg) and the product of Step A as the appropriate alkylating agent, 12 mg of the desired product was obtained. HRMS-ESI (m/z): [M+H]⁺ calcd for C₆₀H₇₀N₁₁O₈S 1104.5130. found: 1104.5125.

### Example 15: 2-[3-(1,3-benzothiazol-2-ylamino)-4-methyl-6,7-dihydro-5H-pyrido[2,3-c]pyridazin-8-yl]-5-[3-[2-fluoro-4-[3-[4-[7-[[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-7-oxo-heptanoyl]piperazin-1-yl]prop-1-ynyl]phenoxy]propyl] thiazole-4-carboxylic acid

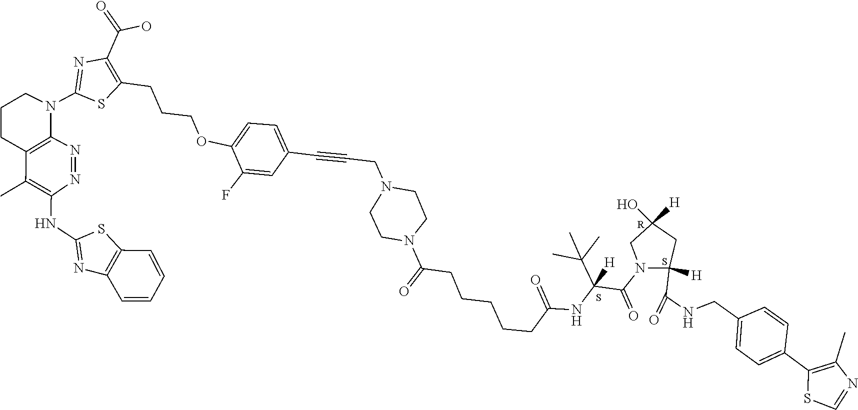

### Step A: 7-[[(1S)-1-[(2S,4R)-4-hydroxy-2-[[4-(4-methylthiazol-5-yl)phenyl]methyl carbamoyl]pyrrolidine-1-carbonyl]-2,2-dimethyl-propyl]amino]-7-oxo-heptanoic acid

Using the General procedure for the acylation and deprotection of VHL ligands starting from (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide, hydrogen chloride (1:1) (200 mg), and using 7-tert-butoxy-7-oxo-heptanoic acid as the appropriate carboxylic acid, 55 mg of the desired product was obtained. ¹H NMR (500 MHz, DMSO-d₆) δ ppm 8.98 (s, 1H), 8.58 (t, 1H), 7.87 (d, 1H), 7.42 (d, 2H), 7.38 (d, 2H), 4.53 (d, 1H), 4.43/4.21 (dd+dd, 2H), 4.42 (m, 1H), 4.34 (m, 1H), 3.67/3.63 (dd+brd, 2H), 2.44 (s, 3H), 2.24/2.11 (dd+dd, 2H), 2.17 (m, 2H), 2.04/2.02/1.91/1.88 (d/d+dd/dd, 2H), 1.55-1.19 (m, 6H), 0.93 (s, 9H); ¹³C NMR (125 MHz, DMSO-d₆) δ ppm 174.9, 172.5, 172.4, 170.2, 152.0, 129.1, 127.9, 69.3, 59.1, 56.8, 56.7, 42.1, 38.4, 35.2, 34.0, 28.7/25.6/24.7, 26.8, 16.4; HRMS-ESI (m/z): [M+H]⁺ calcd for C₂₉H₄₁N₄O₆S: 573.2747. found: 573.2745.

### Step B: 2-[3-(1,3-benzothiazol-2-ylamino)-4-methyl-6,7-dihydro-5H-pyrido[2,3-c]pyridazin-8-yl]-5-[3-[2-fluoro-4-[3-[4-[7-[[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-7-oxo-heptanoyl]piperazin-1-yl]prop-1-ynyl]phenoxy]propyl] thiazole-4-carboxylic acid

Using the General procedure for production of degraders via acylation starting from Preparation 9 (20 mg) and the product of Step A as the appropriate acid, the desired product was obtained (20 mg). HRMS-ESI (m/z): [M+2H]²⁺ calcd for C₄H₇₅FN₁₂O₈S₃: 627.2489. found: 627.2488.

### Example 16: 2-[3-(1,3-benzothiazol-2-ylamino)-4-methyl-6,7-dihydro-5H-pyrido[2,3-c]pyridazin-8-yl]-5-[3-[2-fluoro-4-[3-[4-[8-[[(1S)-1-[(2S,4R)-4-hydroxy-2-[[(1S)-1-[4-(4-methyl thiazol-5-yl)phenyl]ethyl]carbamoyl]pyrrolidine-1-carbonyl]-2,2-dimethyl-propyl]amino]-8-oxo-octanoyl]piperazin-1-yl]prop-1-ynyl]phenoxy]propyl]thiazole-4-carboxylic acid

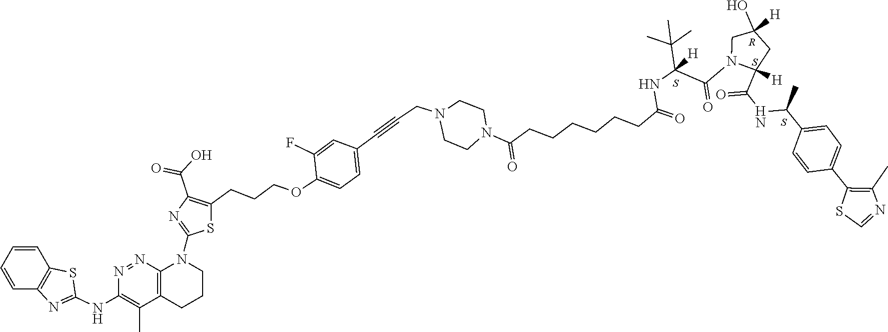

### Step A: 8-[[(1S)-1-[(2S,4R)-4-hydroxy-2-[[(1S)-1-[4-(4-methylthiazol-5-yl)phenyl] ethyl]carbamoyl]pyrrolidine-1-carbonyl]-2,2-dimethyl-propyl]amino]-8-oxo-octanoic acid

Using the General procedure for the acylation and deprotection of VHL ligands starting from (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide, hydrogen chloride (1:1) (500 mg) and 8-tert-butoxy-8-oxo-octanoic acid as the appropriate carboxylic acid, 640 mg of the desired product was obtained. ¹H NMR (500 MHz, DMSO-d₆) δ ppm 12.04 (brs, 1H), 8.99 (s, 1H), 8.38 (d, 1H), 7.8 (d, 1H), 7.43 (m, 2H), 7.38 (m, 2H), 4.91 (m, 1H), 4.51 (d, 1H), 4.41 (t, 1H), 4.27 (m, 1H), 3.61/3.58 (m+m, 2H), 2.45 (s, 3H), 2.23/2.1 (m, 2H), 2.18 (t, 2H), 2.00/1.78 (m+m, 2H), 1.56-1.40 (m, 4H), 1.37 (d, 3H), 1.31-1.19 (m, 4H), 0.93 (s, 9H); ¹³C NMR (125 MHz, DMSO-d₆) δ ppm 152.0, 129.3, 126.8, 69.2, 59.0, 56.8, 56.8, 48.2, 38.2, 34.1, 26.9, 22.9, 16.4; HRMS-ESI (m/z): [M+H]⁺ calcd for C₃₁H₄₅N₄O₆S: 601.3060. found: 601.3051.

### Step B: 2-[3-(1,3-benzothiazol-2-ylamino)-4-methyl-6,7-dihydro-5H-pyrido[2,3-c]pyridazin-8-yl]-5-[3-[2-fluoro-4-[3-[4-[8-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-8-oxo-octanoyl]piperazin-1-yl]prop-1-ynyl]phenoxy]propyl]thiazole-4-carboxylic acid

Using the General procedure for production of degraders via acylation starting from Preparation 9 (80 mg) and the product of Step A as the appropriate acid, the desired product was obtained (30 mg). HRMS-ESI (m/z): [M+2H]²⁺ calcd for C₆₆H₇₉FN₁₂O₈S₃: 641.2645. found: 641.2639.

### Example 17: 2-[3-(1,3-benzothiazol-2-ylamino)-4-methyl-6,7-dihydro-5H-pyrido[2,3-c]pyridazin-8-yl]-5-[3-[2-fluoro-4-[3-[4-[7-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-7-oxo-heptanoyl]piperazin-1-yl]prop-1-ynyl]phenoxy]propyl] thiazole-4-carboxylic acid

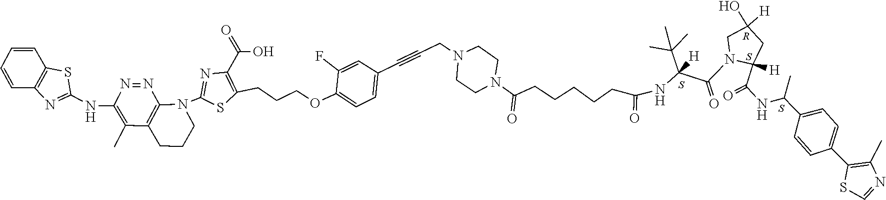

### Step A: 7-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-7-oxo-heptanoic acid

Using the General procedure for the acylation and deprotection of VHL ligands starting from (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide, hydrogen chloride (1:1) (500 mg) and 7-tert-butoxy-7-oxo-heptanoic acid as the appropriate carboxylic acid, 590 mg of the desired product was obtained. ¹H NMR (500 MHz, DMSO-d₆) δ ppm 11.84 (vbrs, 1H), 9.00 (s, 1H), 8.38 (d, 1H), 7.81 (d, 1H), 7.43 (m, 2H), 7.38 (m, 2H), 4.91 (m, 1H), 4.51 (d, 1H), 4.41 (t, 1H), 4.27 (m, 1H), 3.61/3.58 (m+m, 2H), 2.45 (s, 3H), 2.23/2.11 (m+m, 2H), 2.18 (t, 2H), 2.00/1.78 (m+m, 2H), 1.55-1.41 (m, 4H), 1.37 (d, 3H), 1.24 (m, 2H), 0.93 (s, 9H); ¹³C NMR (125 MHz, DMSO-d₆) δ ppm 152.0, 129.3, 126.8, 69.2, 59.0, 56.8, 56.7, 48.2, 38.2, 35.2, 34.0, 28.7, 26.9, 22.9, 16.4; HRMS-ESI (m/z): [M+H]⁺ calcd for C₃₀H₄₃N₄O₆S: 587.2903. found: 587.2899.

### Step B: 2-[3-(1,3-benzothiazol-2-ylamino)-4-methyl-6,7-dihydro-5H-pyrido[2,3-c]pyridazin-8-yl]-5-[3-[2-fluoro-4-[3-[4-[7-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-7-oxo-heptanoyl]piperazin-1-yl]prop-1-ynyl]phenoxy]propyl] thiazole-4-carboxylic acid

Using the General procedure for production of degraders via acylation starting from Preparation 9 (80 mg) and the product of Step A as the appropriate acid, the desired product was obtained (10 mg). HRMS-ESI (m/z): [M+2H]²⁺ calcd for C₆₅H₇₇FN₁₂O₈S₃: 634.2567. found: 634.2559.

### Example 18: 2-[3-(1,3-benzothiazol-2-ylamino)-4-methyl-6,7-dihydro-5H-pyrido[2,3-c]pyridazin-8-yl]-5-[3-[2-fluoro-4-[3-[4-[5-[[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-5-oxo-pentanoyl]piperazin-1-yl]prop-1-ynyl]phenoxy]propyl] thiazole-4-carboxylic acid

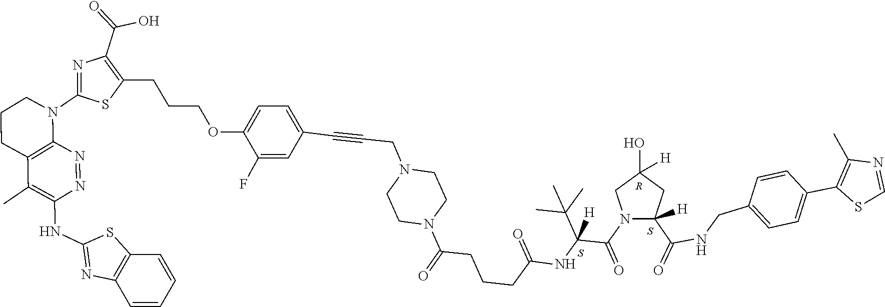

### Step A: 5-[[(1S)-1-[(2S,4R)-4-hydroxy-2-[[4-(4-methylthiazol-5-yl)phenyl]methyl carbamoyl] pyrrolidine-1-carbonyl]-2,2-dimethyl-propyl]amino]-5-oxo-pentanoic acid

Using the General procedure for the acylation and deprotection of VHL ligands starting from (2S,4R)-1-[(2S)-2-amino-3,3-dimethyl-butanoyl]-4-hydroxy-N-[[4-(4-methylthiazol-5-yl) phenyl]methyl]pyrrolidine-2-carboxamide, hydrogen chloride (1:1) (100 mg) and 5-tert-butoxy-5-oxo-pentanoic acid as the appropriate carboxylic acid, 39 mg of the desired product was obtained. ¹H NMR (500 MHz, DMSO-d₆) δ ppm 12.04 (brs, 1H), 8.99 (s, 1H), 8.58 (t, 1H), 7.91 (d, 1H), 7.42 (d, 2H), 7.38 (d, 2H), 4.65 (brs, 1H), 4.54 (d, 1H), 4.43/4.22 (dd+dd, 2H), 4.42 (t, 1H), 4.35 (m, 1H), 3.67/3.64 (dd+dd, 2H), 2.44 (s, 3H), 2.28/2.24 (t/t, 2H), 2.19/2.16 (t/t, 2H), 2.03/1.90 (m+m, 2H), 1.70 (qn, 2H), 0.93 (s, 9H); ¹³C NMR (125 MHz, DMSO-d₆) δ ppm 152.0, 129.1, 127.9, 69.4, 59.1, 56.9, 56.8, 42.1, 38.4, 35.7/33.2, 34.5/33.6, 26.8, 21.3, 16.4; HRMS-ESI (m/z): [M+H]⁺ calcd for C₂₇H₃₇N₄O₆S: 545.2434. found: 545.2435.

### Step B: 2-[3-(1,3-benzothiazol-2-ylamino)-4-methyl-6,7-dihydro-5H-pyrido[2,3-c]pyridazin-8-yl]-5-[3-[2-fluoro-4-[3-[4-[5-[[(1S)-1-[(2S,4R)-4-hydroxy-2-[[4-(4-methylthiazol-5-yl)phenyl] methylcarbamoyl]pyrrolidine-1-carbonyl]-2,2-dimethyl-propyl]amino]-5-oxo-pentanoyl] piperazin-1-yl]prop-1-ynyl]phenoxy]propyl] thiazole-4-carboxylic acid

Using the General procedure for production of degraders via acylation starting from Preparation 9 (37 mg) and the product of Step A as the appropriate acid, the desired product was obtained (10 mg). HRMS-ESI (m/z): [M+H]⁺ calcd for C₆₂H₇₀FN₁₂O₈S₃: 1225.4586. found: 1225.4578.

### Example 19: 2-[3-(1,3-benzothiazol-2-ylamino)-4-methyl-6,7-dihydro-5H-pyrido[2,3-c]pyridazin-8-yl]-5-[3-[2-fluoro-4-[3-[4-[6-[[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-6-oxo-hexanoyl]piperazin-1-yl]prop-1-ynyl]phenoxy]propyl]thiazole-4-carboxylic acid

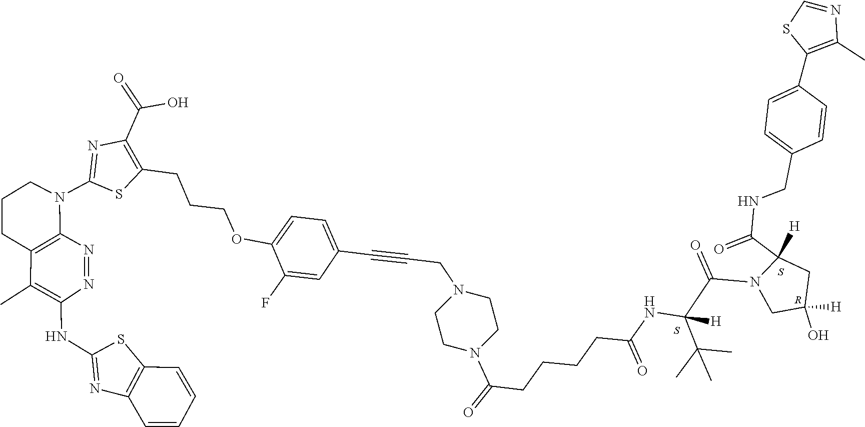

### Step A: 6-[[(1S)-1-[(2S,4R)-4-hydroxy-2-[[4-(4-methylthiazol-5-yl)phenyl]methylcarbamoyl] pyrrolidine-1-carbonyl]-2,2-dimethyl-propyl]amino]-6-oxo-hexanoic acid

Using the General procedure for the acylation and deprotection of VHL ligands starting from (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide, hydrogen chloride (1:1) (200 mg) and 6-tert-butoxy-6-oxo-hexanoic acid as the appropriate carboxylic acid, 232 mg of the desired product was obtained. ¹H NMR (500 MHz, DMSO-d₆) δ ppm 8.99 (s, 1H), 8.61 (t, 1H), 7.89 (d, 1H), 7.42 (d, 2H), 7.38 (d, 2H), 4.53 (d, 1H), 4.43/4.21 (dd+dd, 2H), 4.42 (m, 1H), 4.34 (m, 1H), 3.67/3.62 (dd+d, 2H), 2.44 (s, 3H), 2.26/2.13 (dd+dd, 2H), 2.20 (m, 2H), 2.04/2.02/1.9/1.88 (d/d+dd/dd, 2H), 1.54-1.41 (m, 4H), 0.93 (s, 9H); ¹³C NMR (125 MHz, DMSO-d₆) δ ppm 174.9, 172.4, 172.3, 170.1, 152.0, 129.1, 127.9, 69.3, 59.2, 56.8, 56.8, 42.1, 38.4, 35.0, 33.9, 26.9, 25.5/24.6, 16.4; HRMS-ESI (m/z): [M+H]⁺ calcd for C₂₈H₃₉N₄O₆S: 559.2590. found: 559.2587.

### Step B: 2-[3-(1,3-benzothiazol-2-ylamino)-4-methyl-6,7-dihydro-5H-pyrido[2,3-c]pyridazin-8-yl]-5-[3-[2-fluoro-4-[3-[4-[6-[[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-6-oxo-hexanoyl]piperazin-1-yl]prop-1-ynyl]phenoxy]propyl] thiazole-4-carboxylic acid

Using the General procedure for production of degraders via acylation starting from Preparation 9 (50 mg) and the product of Step A as the appropriate acid, the desired product was obtained (45 mg). HRMS-ESI (m/z): [M+H]⁺ calcd for C₆₃H₇₂FN₁₂O₈S₃: 1239.4742. found: 1239.4743.

### Example 20: 2-[3-(1,3-benzothiazol-2-ylamino)-4-methyl-6,7-dihydro-5H-pyrido[2,3-c]pyridazin-8-yl]-5-[3-[2-fluoro-4-[3-[4-[8-[[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-8-oxo-octanoyl]piperazin-1-yl]prop-1-ynyl]phenoxy]propyl]thiazole-4-carboxylic acid

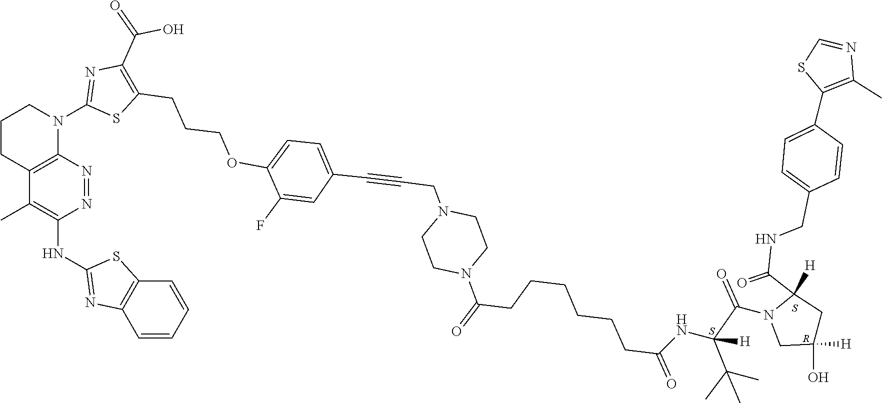

### Step A: 8-[[(1S)-1-[(2S,4R)-4-hydroxy-2-[[4-(4-methylthiazol-5-yl)phenyl]methyl carbamoyl]pyrrolidine-1-carbonyl]-2,2-dimethyl-propyl]amino]-8-oxo-octanoic acid

Using the General procedure for the acylation and deprotection of VHL ligands starting from (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide, hydrogen chloride (1:1) (200 mg) and 8-tert-butoxy-8-oxo-octanoic acid as the appropriate carboxylic acid, 33 mg of the desired product was obtained. ¹H NMR (500 MHz, DMSO-d₆) δ ppm 8.99 (s, 1H), 8.57 (t, 1H), 7.86 (d, 1H), 7.42 (d, 2H), 7.38 (d, 2H), 4.54 (d, 1H), 4.43/4.21 (dd+dd, 2H), 4.42 (m, 1H), 4.34 (br., 1H), 3.67/3.63 (dd+brd, 2H), 2.44 (s, 3H), 2.24/2.10 (m+m, 2H), 2.18 (t, 2H), 2.04/2.02/1.91/1.88 (d/d+dd/dd, 2H), 1.54-1.38/1.30-1.18 (m+m, 8H), 0.93 (s, 9H); ¹³C NMR (125 MHz, DMSO-d₆) δ ppm 175.0, 172.5, 172.4, 170.2, 152.0, 129.1, 127.9, 69.3, 59.1, 56.8, 56.7, 42.1, 38.4, 35.3, 34.1, 28.9/28.8/25.8/24.9, 26.8, 16.4; HRMS-ESI (m/z): [M+H]⁺ calcd for C₃₀H₄₃N₄O₆S: 587.2903. found: 587.2899.

### Step B: 2-[3-(1,3-benzothiazol-2-ylamino)-4-methyl-6,7-dihydro-5H-pyrido[2,3-c]pyridazin-8-yl]-5-[3-[2-fluoro-4-[3-[4-[8-[[(1S)-1-[(2S,4R)-4-hydroxy-2-[[4-(4-methylthiazol-5-yl)phenyl] methylcarbamoyl]pyrrolidine-1-carbonyl]-2,2-dimethyl-propyl]amino]-8-oxo-octanoyl] piperazin-1-yl]prop-1-ynyl]phenoxy]propyl]thiazole-4-carboxylic acid

Using the General procedure for production of degraders via acylation starting from Preparation 9 (25 mg) and the product of Step A as the appropriate acid, the desired product was obtained (15 mg). HRMS-ESI (m/z): [M+H]⁺ calcd for C₆₅H₇₆FN₁₂O₈S₃: 1267.5055. found: 1267.5037.

### Example 21: 2-[3-(1,3-benzothiazol-2-ylamino)-4-methyl-6,7-dihydro-5H-pyrido[2,3-c]pyridazin-8-yl]-5-[3-[2-fluoro-4-[3-[4-[4-[[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-4-oxo-butanoyl]piperazin-1-yl]prop-1-ynyl]phenoxy]propyl]thiazole-4-carboxylic acid

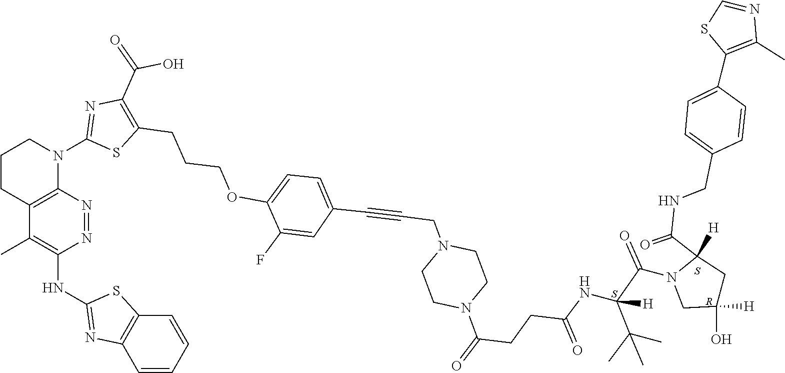

### Step A: 4-[[(1S)-1-[(2S,4R)-4-hydroxy-2-[[4-(4-methylthiazol-5-yl)phenyl]methyl carbamoyl]pyrrolidine-1-carbonyl]-2,2-dimethyl-propyl]amino]-4-oxo-butanoic acid

Using the General procedure for the acylation and deprotection of VHL ligands starting from (2S,4R)-1-[(2S)-2-amino-3,3-dimethyl-butanoyl]-4-hydroxy-N-[[4-(4-methylthiazol-5-yl) phenyl]methyl]pyrrolidine-2-carboxamide, hydrogen chloride (1:1) (100 mg), and 4-tert-butoxy-4-oxo-butanoic acid as the appropriate carboxylic acid, 52 mg of the desired product was obtained. MS-ESI (m/z): 531 [M+H]⁺.

### Step B: 2-[3-(1,3-benzothiazol-2-ylamino)-4-methyl-6,7-dihydro-5H-pyrido[2,3-c]pyridazin-8-yl]-5-[3-[2-fluoro-4-[3-[4-[4-[[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-4-oxo-butanoyl]piperazin-1-yl]prop-1-ynyl]phenoxy]propyl]thiazole-4-carboxylic acid

Using the General procedure for production of degraders via acylation starting from Preparation 9 (40 mg) and the product of Step A as the appropriate acid, the desired product was obtained (25 mg). HRMS-ESI (m/z): [M+H]⁺ calcd for C₆₁H₆₈FN₁₂O₈S₃: 1211.4429. found: 1211.4427.

### Example 22: 6-[3-(1,3-benzothiazol-2-ylamino)-4-methyl-6,7-dihydro-5H-pyrido[2,3-c]pyridazin-8-yl]-3-[1-[[3-[2-[4-[7-[[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-7-oxo-heptanoyl]piperazin-1-yl]ethoxy]-5,7-dimethyl-1-adamantyl]methyl]-5-methyl-pyrazol-4-yl]pyridine-2-carboxylic acid

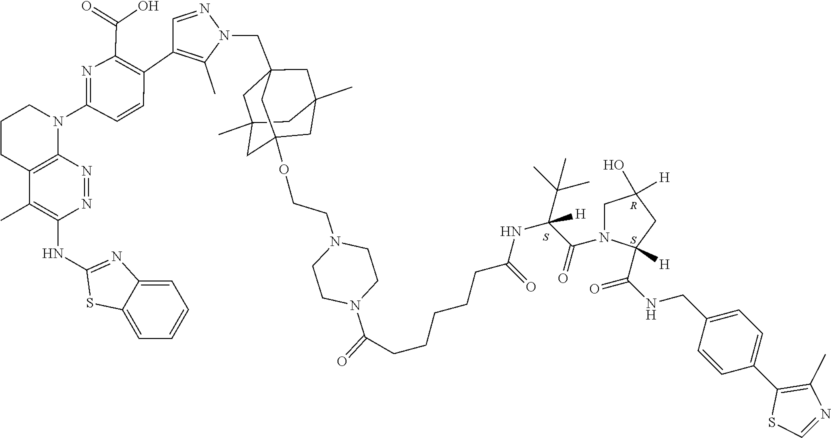

Using the General procedure for production of degraders via acylation starting from Preparation 5 (40 mg) and the product of Step A of Example 15 as the appropriate acid, the desired product was obtained (18 mg). HRMS-ESI (m/z): [M+H]⁺ calcd for C₇₃H₉₃N₁₄O₈S₂: 1357.6742. found: 1357.6740.

### Example 23: 6-[3-(1,3-benzothiazol-2-ylamino)-4-methyl-6,7-dihydro-5H-pyrido[2,3-c]pyridazin-8-yl]-3-[1-[[3-[2-[4-[6-[[(1S)-1-[(2S,4R)-4-hydroxy-2-[[4-(4-methylthiazol-5-yl) phenyl]methylcarbamoyl]pyrrolidine-1-carbonyl]-2,2-dimethyl-propyl]amino]-6-oxo-hexanoyl]piperazin-1-yl]ethoxy]-5,7-dimethyl-1-adamantyl]methyl]-5-methyl-pyrazol-4-yl]pyridine-2-carboxylic acid

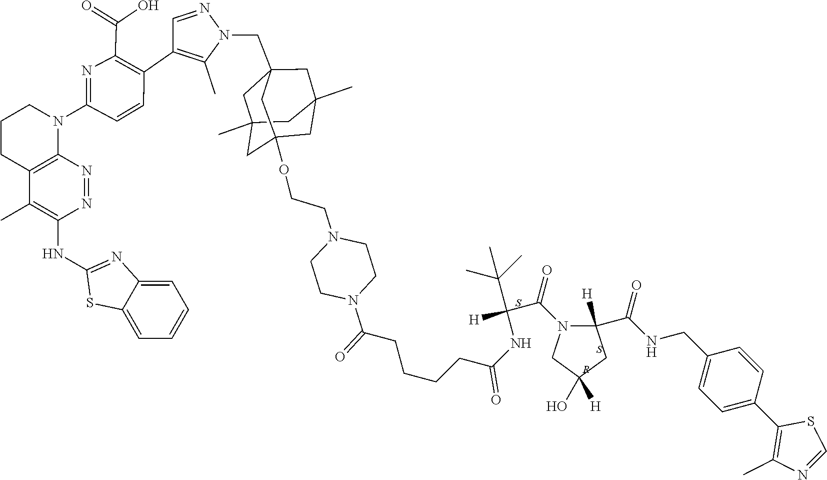

Using the General procedure for production of degraders via acylation starting from Preparation 5 (40 mg) and the product of Step A of Example 19 as the appropriate acid, the desired product was obtained (55 mg). HRMS-ESI (m/z): [M+H]⁺ calcd for C₇₂H₉₁N₁₄O₈S₂: 1343.6586. found: 1343.6582.

### Example 24: 6-[3-(1,3-benzothiazol-2-ylamino)-4-methyl-6,7-dihydro-5H-pyrido[2,3-c]pyridazin-8-yl]-3-[1-[[3-[2-[4-[8-[[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-8-oxo-octanoyl]piperazin-1-yl]ethoxy]-5,7-dimethyl-1-adamantyl]methyl]-5-methyl-pyrazol-4-yl]pyridine-2-carboxylic acid

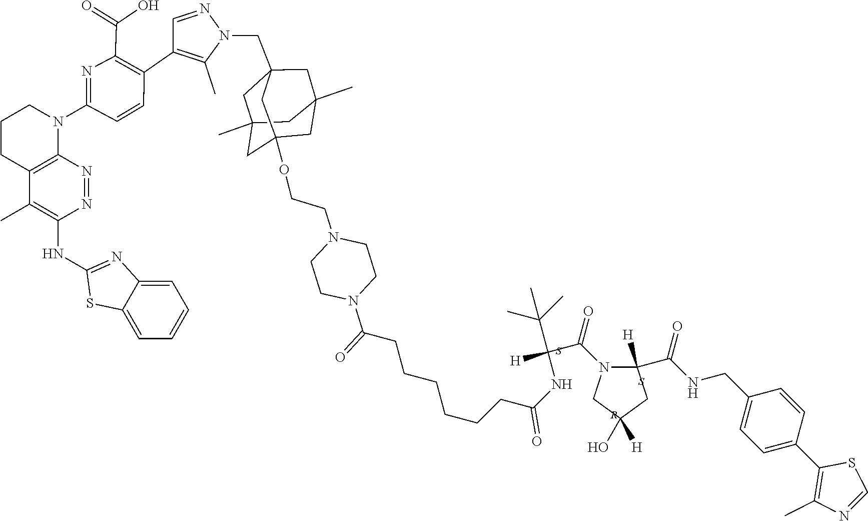

Using the General procedure for production of degraders via acylation starting from Preparation 5 (40 mg) and the product of Step A of Example 20 as the appropriate acid, the desired product was obtained (20 mg). HRMS-ESI (m/z): [M+H]⁺ calcd for C₇₄H₉₅N₁₄O₈S₂: 1371.6899. found: 1371.6894.

### Example 25: 2-[3-(1,3-benzothiazol-2-ylamino)-4-methyl-6,7-dihydro-5H-pyrido[2,3-c]pyridazin-8-yl]-5-[3-[4-[3-[4-[8-[2-[2-[[2-[2-(2,6-dioxo-3-piperidyl)-1,3-dioxo-isoindolin-4-yl]oxyacetyl]amino]ethoxy]ethylamino]-8-oxo-octanoyl]piperazin-1-yl]prop-1-ynyl]-2-fluoro-phenoxy]propyl]thiazole-4-carboxylic acid

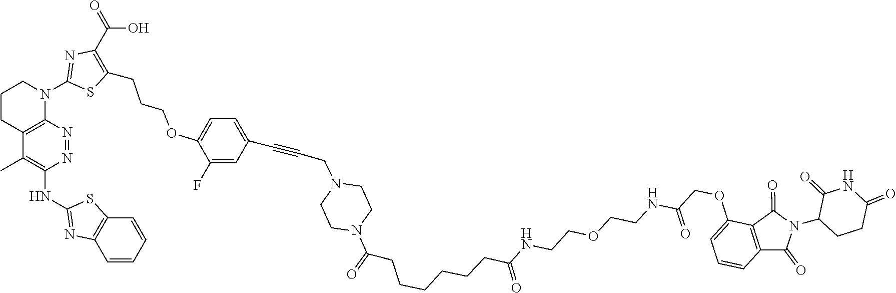 [SMILES: Cc1c(Nc2nc3ccccc3s2)nnc2c1CCCN2c1nc(C(=O)O)c(CCCOc2ccc(C#CCN3CCN(C(=O)CCCCCCC(=O)NCCOCCNC(=O)COc4cccc5c4C(=O)N(C4CCC(=O)NC4=O)C5=O)CC3)cc2F)s1]

### Step A: 8-[2-[2-[[2-[2-(2,6-dioxo-3-piperidyl)-1,3-dioxo-isoindolin-4-yl]oxyacetyl]amino]ethoxy]ethylamino]-8-oxo-octanoic acid

After stirring the mixture of 8-tert-butoxy-8-oxo-octanoic acid (143 mg, 0.62 mmol, 1.3 eq), HATU (200 mg, 0.53 mmol, 1.1 eq), triethylamine (0.33 mL, 2.39 mmol, 5 eq) in DCM (2.4 mL) for 20 min, N-[2-(2-aminoethoxy)ethyl]-2-[2-(2,6-dioxo-3-piperidyl)-1,3-dioxo-isoindolin-4-yl]oxy-acetamide (200 mg, 0.48 mmol) was added, and the resulting mixture was stirred for 1 h. After purification on preparative HPLC (Teledyne EZ) (C18, 0.1% aqueous TFA and MeCN as eluents), the intermediate was dissolved in DCM (2.4 mL) and TFA (2.4 mL) and stirred for 1 h. Volatiles were removed under reduced pressure to get the desired product (208 mg, 75%). ¹H NMR (500 MHz, DMSO-d₆) δ ppm 11.96 (br s, 1H), 11.12 (s, 1H), 8.02 (t, 1H), 7.82 (dd, 1H), 7.78 (t, 1H), 7.50 (d, 1H), 7.40 (d, 1H), 5.13 (dd, 1H), 4.79 (s, 2H), 3.45 (t, 2H), 3.40 (t, 2H), 3.31 (m, 2H), 3.20 (m, 2H), 2.90/2.59 (m+m, 2H), 2.54/2.04 (m+m, 2H), 2.17 (t, 2H), 2.03 (t, 2H), 1.52-1.38 (m, 4H), 1.29-1.15 (m, 4H); HRMS-ESI (m/z): [M+H]⁺ calcd for C₂₇H₃₅N₄O₁₀: 575.2353. found: 575.2351.

### Step B: 2-[3-(1,3-benzothiazol-2-ylamino)-4-methyl-6,7-dihydro-5H-pyrido[2,3-c]pyridazin-8-yl]-5-[3-[4-[3-[4-[8-[2-[2-[[2-[2-(2,6-dioxo-3-piperidyl)-1,3-dioxo-isoindolin-4-yl]oxyacetyl]amino]ethoxy]ethylamino]-8-oxo-octanoyl]piperazin-1-yl]prop-1-ynyl]-2-fluoro-phenoxy]propyl]thiazole-4-carboxylic acid

Using the General procedure for production of degraders via acylation starting from Preparation 9 (50 mg) and the product of Step A as the appropriate acid, the desired product was obtained (16 mg). HRMS-ESI (m/z): [M+H]⁺ calcd for C₆₂H₆₈FN₁₂O₁₂S₂: 1255.4505. found: 1255.4509.

### Example 26: 2-[3-(1,3-benzothiazol-2-ylamino)-4-methyl-6,7-dihydro-5H-pyrido[2,3-c]pyridazin-8-yl]-5-[3-[4-[3-[4-[5-[2-[2-[[2-[2-(2,6-dioxo-3-piperidyl)-1,3-dioxo-isoindolin-4-yl]oxyacetyl]amino]ethoxy]ethylamino]-5-oxo-pentanoyl]piperazin-1-yl]prop-1-ynyl]-2-fluoro-phenoxy]propyl]thiazole-4-carboxylic acid

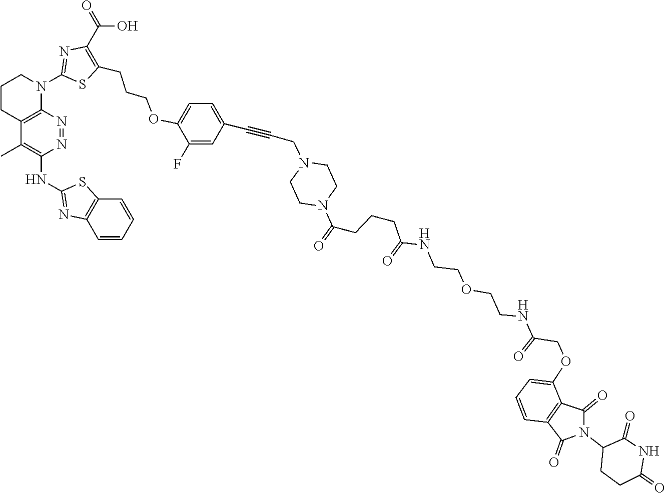 [SMILES: Cc1c(Nc2nc3ccccc3s2)nnc2c1CCCN2c1nc(C(=O)O)c(CCCOc2ccc(C#CCN3CCN(C(=O)CCCC(=O)NCCOCCNC(=O)COc4cccc5c4C(=O)N(C4CCC(=O)NC4=O)C5=O)CC3)cc2F)s1]

### Step A: 5-[2-[2-[[2-[2-(2,6-dioxo-3-piperidyl)-1,3-dioxo-isoindolin-4-yl]oxyacetyl] amino]ethoxy]ethylamino]-5-oxo-pentanoic acid

To the mixture of N-[2-(2-aminoethoxy)ethyl]-2-[2-(2,6-dioxo-3-piperidyl)-1,3-dioxo-isoindolin-4-yl]oxy-acetamide (200 mg, 0.48 mmol) and triethylamine (0.20 mL, 1.4 mmol, 3 eq) in DCM (2.4 mL) was added tetrahydropyran-2,6-dione (65 mg, 0.57 mmol, 1.2 eq). The reaction was stirred for 18 h. The crude product was purified by preparative HPLC (Teledyne EZ) (C18, 0.1% aqeuous TFA, MeCN) to give the desired product. ¹H NMR (500 MHz, DMSO-d₆) δ ppm 12.02 (br., 1H), 11.12 (s, 1H), 8.02 (t, 1H), 7.83 (t, 1H), 7.81 (dd, 1H), 7.49 (d, 1H), 7.39 (d, 1H), 5.12 (dd, 1H), 4.79 (s, 2H), 3.45 (t, 2H), 3.40 (t, 2H), 3.31 (q, 2H), 3.20 (q, 2H), 2.90/2.59 (ddd+dm, 2H), 2.53/2.04 (m+m, 2H), 2.18 (t, 2H), 2.08 (t, 2H), 1.68 (quint., 2H); ¹³C NMR (125 MHz, DMSO-d₆) δ ppm 174.7, 173.3/170.4, 172.2, 167.4, 167.2/165.9, 137.4, 120.8, 116.5, 69.4, 69.0, 67.9, 49.3, 38.8, 38.8, 34.8, 33.4, 31.4, 22.5, 21.1; HRMS-ESI (m/z): [M+H]⁺ calcd for C₂₄H₂₉N₄O₁₀: 533.1884. found: 533.1881.

### Step B: 2-[3-(1,3-benzothiazol-2-ylamino)-4-methyl-6,7-dihydro-5H-pyrido[2,3-c]pyridazin-8-yl]-5-[3-[4-[3-[4-[5-[2-[2-[[2-[2-(2,6-dioxo-3-piperidyl)-1,3-dioxo-isoindolin-4-yl]oxyacetyl] amino]ethoxy]ethylamino]-5-oxo-pentanoyl]piperazin-1-yl]prop-1-ynyl]-2-fluoro-phenoxy]propyl]thiazole-4-carboxylic acid

Using the General procedure for production of degraders via acylation starting from Preparation 9 (50 mg) and the product of Step A as the appropriate acid, the desired product was obtained (18 mg). HRMS-ESI (m/z): [M+H]⁺ calcd for C₅₉H₆₂FN₁₂O₁₂S₂: 1213.4036. found: 1213.4031.

### Example 27: 2-[[6-(1,3-benzothiazol-2-ylamino)-5-methyl-pyridazin-3-yl]-[3-[1-[2-[2-[2-[2-[[2-[2-(2,6-dioxo-3-piperidyl)-1,3-dioxo-isoindolin-4-yl]oxyacetyl]amino] ethoxy]ethoxy]ethoxy]ethyl]triazol-4-yl]propyl]amino]-5-[3-[2-fluoro-4-[3-(methylamino)prop-1-ynyl]phenoxy]propyl]thiazole-4-carboxylic acid

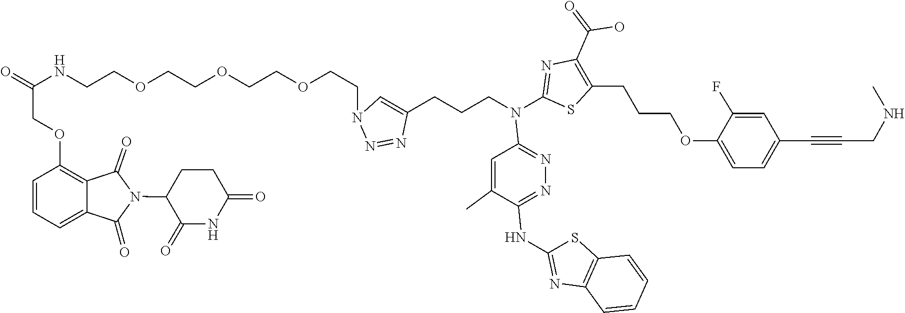

### Step A: N-[2-[2-[2-(2-azidoethoxy)ethoxy]ethoxy]ethyl]-2-[2-(2,6-dioxo-3-piperidyl)-1,3-dioxo-isoindolin-4-yl]oxy-acetamide

To a solution of 2-[2-(2,6-dioxo-3-piperidyl)-1,3-dioxo-isoindolin-4-yl]oxyacetic acid (247 mg, 0.743 mmol) in DCM (1 mL) was added a solution of 2-[2-[2-(2-azidoethoxy)ethoxy]ethoxy] ethanamine (136 mg, 0.780 mmol) in DCM (1 mL) followed by EDC (149 mg, 0.780 mmol), HOBt (105 mg, 0.780 mmol), and DIEA (431 μL, 2.602 mmol). The reaction was stirred at room temperature for 24 h. After addition of 0.1 N HCl the reaction was extracted with DCM. The organic phases were combined and washed with saturated NaHCO₃ and brine, dried over MgSO₄, and evaporated to dryness under vacuum. The residue was purified by chromatography on silica gel in using a gradient of [A: AcOEt] in [B: AcOEt/EtOH 8/2] to give 236 mg (0.443 mmol) of the expected product yield: 59%. ¹H NMR (400 MHz, DMSO-d₆) δ ppm 11.10 (sl, 1H), 8.00 (tl, 1H), 7.82 (t, 1H), 7.50 (d, 1H), 7.40 (d, 1H), 5.12 (dd, 1H), 4.79 (s, 2H), 3.61-3.35 (m, 14H), 3.32 (quad, 2H), 2.89/2.56 (m, 2H), 2.56/2.04 (m, 2H); IR: 3700-2800, 2104, 1773/1709/1668, 1115.

### Step B: 2-[[6-(1,3-benzothiazol-2-ylamino)-5-methyl-pyridazin-3-yl]-[3-[1-[2-[2-[2-[2-[[2-[2-(2,6-dioxo-3-piperidyl)-1,3-dioxo-isoindolin-4-yl]oxyacetyl]amino]ethoxy]ethoxy]ethoxy] ethyl]triazol-4-yl]propyl]amino]-5-[3-[2-fluoro-4-[3-(methylamino) prop-1-ynyl]phenoxy] propyl]thiazole-4-carboxylic acid

To a solution of CuSO₄×5 H₂O (9.87 mg, 0.056 mmol) in water (1.2 mL) was added THTPA (24.13 mg, 0.056 mmol). This solution was added to a suspension containing the product of Step A (32.53 mg, 1.1 eq), the product of Preparation 10 (50 mg, 0.05554 mmol), and Na-L-ascorbate (11.00 mg, 0.056 mmol) in DMSO (3 mL). The reaction was heated at 85° C. for 3 h. After the addition of a 4N HCl in dioxane (694 μL, 2.77 mmol), the mixture was stirred at 80° C. for 6 h, then at RT overnight. The reaction was filtered, and the filtrate was injected directly on Xbridge column for purification according to TFA method. After lyophilization the expected product was obtained (43 mg, 66%). HRMS-ESI (m/z): [M+H]⁺ calcd for C₅₇H₆₁FN₁₃O₁₂S₂: 1202.3988; found 1202.3952.

### Example 28: 6-[[6-(1,3-benzothiazol-2-ylamino)-5-methyl-pyridazin-3-yl]-[5-[4-[2-[2-[2-[[2-(2,6-dioxo-3-piperidyl)-1-oxo-isoindolin-4-yl]amino]-2-oxo-ethoxy]ethoxy]ethoxymethyl] triazol-1-yl]pentyl]amino]-3-[1-[[3-[2-(dimethylamino)ethoxy]-5,7-dimethyl-1-adamantyl] methyl]-5-methyl-pyrazol-4-yl]pyridine-2-carboxylic acid

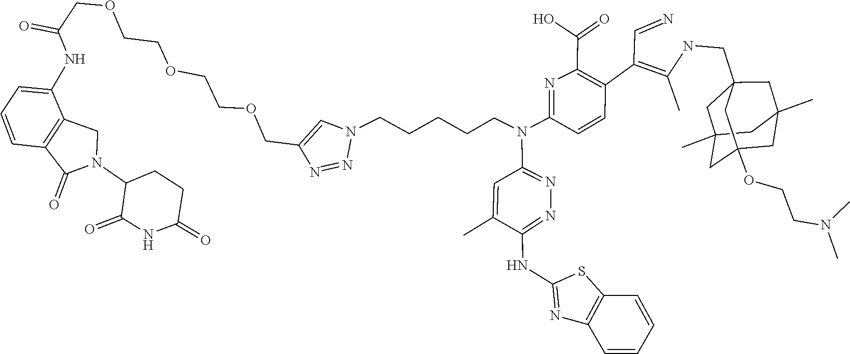

### Step A: tert-butyl 2-[2-(2-prop-2-ynoxyethoxy)ethoxy]acetate

To a solution of 2-(2-prop-2-ynoxyethoxy)ethanol (1 g, 6.94 mmol) in THF (30 ml) was added sodium hydride 60% in mineral oil (282 mg, 7.08 mmol) at 0° C. and the reaction was stirred at 0° C. for 30 min. A solution of tert-butyl 2-bromoacetate (1.54 mL, 10.4 mmol) in THF (5 ml) was added and the reaction was stirred at RT for 2 h. The reaction was quenched with water and extracted with ethyl acetate. The combined organic phases were washed with brine, dried, concentrated and purified by chromatography on silica by eluting with a gradient of [A: petroleum ether] in [B: ethyl acetate] to give the desired product (785 mg, 44%). ¹H NMR (400 MHz, DMSO-d₆) δ ppm 4.18 (s, 2H), 4.00 (s, 2H), 3.55 (m, 8H), 3.40 (t, 1H), 1.40 (s, 9H); IR: 2200, 1745.

### Step B: 2-[2-(2-prop-2-ynoxyethoxy)ethoxy]acetic acid

The product of Step A (785 mg, 3.04 mmol) in DCM was treated with a 4N solution of HCl in dioxane (3.79 mL, 15.2 mmol). The reaction was stirred for 4 h and additional HCl was added (3.79 mL, 15.2 mmol). After 16 h stirring, the reaction was concentrated to give the desired product (470 mg). ¹H NMR (400 MHz, dmso-d6) δ ppm 12.52 (sl, 1H), 4.13 (d, 2H), 4.01 (s, 2H), 3.55 (m, 8H), 3.40 (t, 1H); IR: 3700-2500, 3265, 2116, 1736, 1086.

### Step C: N-[2-(2,6-dioxo-3-piperidyl)-1-oxo-isoindolin-4-yl]-2-[2-(2-prop-2-ynoxy ethoxy) ethoxy]acetamide

To 3-(4-amino-1-oxo-isoindolin-2-yl)piperidine-2,6-dione (0.137 mL, 0.77 mmol) and the product of Step B (155.98 mg, 0.771 mmol) in DMSO (8 ml) were successively added HATU (293.3 mg, 0.77 mmol), HOAt (105.0 mg, 0.77 mmol), and DIEA (0.686 mL, 3.85 mmol) and the mixture was stirred for 1 h. After filtration, the filtrate was injected into an Xbridge for purification according to TFA method. After lyophilization the desired product was obtained (215 mg, 63%). ¹H NMR (400 MHz, DMSO-d₆) δ ppm 10.98 (m, 1H), 9.66 (m, 1H), 7.73 (d, 1H), 7.51 (m, 2H), 5.13 (dd, 1H), 4.36 (dd, 2H), 4.13 (s, 2H), 4.07 (d, 2H), 3.63 (m, 8H), 3.40 (m, 1H), 2.90 (m, 1H), 2.63 (m, 1H), 2.36 (m, 1H), 2.01 (m, 1H).

### Step D: (4-methoxyphenyl)methyl 3-[1-[[3-[2-(dimethylamino)ethoxy]-5,7-dimethyl-1-adamantyl]methyl]-5-methyl-pyrazol-4-yl]-6-[5-[4-[2-[2-[2-[[2-(2,6-dioxo-3-piperidyl)-1-oxo-isoindolin-4-yl]amino]-2-oxo-ethoxy]ethoxy]ethoxymethyl]triazol-1-yl]pentyl-[5-methyl-6-[(Z)-[3-(2-trimethylsilylethoxymethyl)-1,3-benzothiazol-2-ylidene]amino]pyridazin-3-yl]amino]pyridine-2-carboxylate

To CuSO₄ 5H₂O (8.19 mg, 46.1 μmol) in water (0.7 mL) was added Na-L-ascorbate (9.14 mg, 46.1 μmol). After stirring at RT, the mixture was treated with the product of Step C (18.42 mg, 41.5 μmol) and the product of Preparation 7 (25 mg, 23.07 μmol) in 2-methylpropan-2-ol (1.6 mL) and the reaction was heated at 55° C. for 3 h. The reaction was quenched with brine, extracted with DCM. The combined organic layers were dried and concentrated. The crude was purified via flash chromatography on silica gel by elution with a gradient of [A: DCM] in [B: DCM/MeOH/NH₃ 90/10/1%] to give the desired product (31 mg, 88%).

### Step E: 6-[[6-(1,3-benzothiazol-2-ylamino)-5-methyl-pyridazin-3-yl]-[5-[4-[2-[2-[2-[[2-(2,6-dioxo-3-piperidyl)-1-oxo-isoindolin-4-yl]amino]-2-oxo-ethoxy]ethoxy]ethoxymethyl] triazol-1-yl]pentyl]amino]-3-[1-[[3-[2-(dimethylamino)ethoxy]-5,7-dimethyl-1-adamantyl] methyl]-5-methyl-pyrazol-4-yl]pyridine-2-carboxylic acid

To the product of Step D (30 mg, 19.6 μmol) in acetonitrile (0.1 mL) was added pyridine, hydrogen fluoride (1:1) (89 μL, 982 μmol) and the mixture was stirred at 60° C. for 3 h in a closed bottle. The mixture was directly injected in CSH column for purification according to TFA method to give the desired product (15.3 mg, 56%). HRMS-ESI (m/z) [M+H]⁺ calcd for C₆₆H₈₂N₁₅O₁₀S: 1276.6090. found 1276.6020.

### Example 29: 2-[[6-(1,3-benzothiazol-2-ylamino)-5-methyl-pyridazin-3-yl]-[3-[1-[2-[2-[2-[2-[2-[3-[[(1S)-1-[(2S,4R)-4-hydroxy-2-[[4-(4-methylthiazol-5-yl)phenyl]methyl carbamoyl] pyrrolidine-1-carbonyl]-2,2-dimethyl-propyl]amino]-3-oxo-propoxy]ethoxy]ethoxy]ethoxy] ethoxy]ethyl]triazol-4-yl]propyl]amino]-5-[3-[2-fluoro-4-[3-(methylamino)prop-1-ynyl] phenoxy]propyl]thiazole-4-carboxylic acid

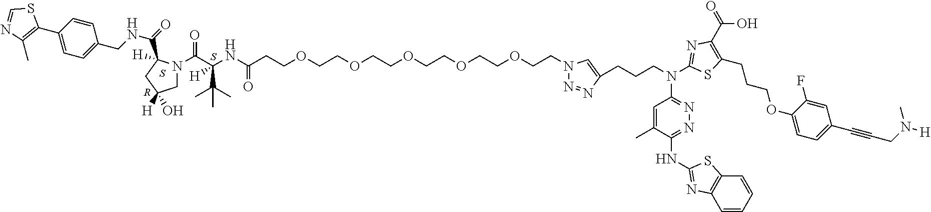

### Step A: (2S,4R)-1-[(2S)-2-[3-[2-[2-[2-[2-(2-azidoethoxy)ethoxy]ethoxy]ethoxy] ethoxy]propanoylamino]-3,3-dimethyl-butanoyl]-4-hydroxy-N-[[4-(4-methylthiazol-5-yl)phenyl]methyl]pyrrolidine-2-carboxamide

To (2,5-dioxopyrrolidin-1-yl) 3-[2-[2-[2-[2-(2-azidoethoxy)ethoxy]ethoxy]ethoxy] ethoxy]propanoate (203.7 mg, 0.47 mmol) in THF (2 mL) was added (2S,4R)-1-[(2S)-2-amino-3,3-dimethyl-butanoyl]-4-hydroxy-N-[[4-(4-methylthiazol-5-yl)phenyl]methyl] pyrrolidine-2-carboxamide;di-hydrochloride (200 mg, 0.428 mmol) and DIEA (0.224 μL, 1.29 mmol) and the solution was stirred for 18 h. After dilution with AcOEt, the organic medium was washed with water and brine, dried, concentrated and purified by flash chromatography on silica gel by elution with a gradient of [A: ethyl acetate] in [B: ethyl acetate/ethanol/NH₃ 80/20/2%] to give the desired product (177 mg, 55%). ¹H NMR (400 MHz, DMSO-d₆) δ ppm 8.99 (s, 1H), 8.55/7.9 (t+d, 2H), 7.40 (2d, 4H), 5.11 (d, 1H), 4.58 (d, 1H), 4.42 (m, 2H), 4.35 (m, 1H), 4.20 (dd, 1H), 3.65 (m, 2H), 3.65-3.45 (m, 20H), 3.40 (t, 2H), 2.55/2.38 (m, 2H), 2.45 (s, 3H), 2.05/1.90 (m, 2H), 0.95 (s, 9H); IR: 3326, 2100, 1667-1629, 1533.

### Step B: 2-[[6-(1,3-benzothiazol-2-ylamino)-5-methyl-pyridazin-3-yl]-[3-[1-[2-[2-[2-[2-[2-[3-[[(1S)-1-[(2S,4R)-4-hydroxy-2-[[4-(4-methylthiazol-5-yl)phenyl]methyl carbamoyl]pyrrolidine-1-carbonyl]-2,2-dimethyl-propyl]amino]-3-oxo-propoxy] ethoxy]ethoxy]ethoxy] ethoxy]ethyl]triazol-4-yl]propyl]amino]-5-[3-[2-fluoro-4-[3-(methylamino)prop-1-ynyl]phenoxy]propyl]thiazole-4-carboxylic acid

To CuSO₄×5 H₂O (9.86 mg, 55.54 μmol) in water (1.2 mL) was added THTPA (24.13 mg, 55.54 μmol). This solution was added to a suspension of the product of Step A (45.69 mg, 61.10 μmol), the product of Preparation 10 (50 mg, 55.54 μmol), and Na-L-ascorbate (11.00 mg, 55.54 μmol) in DMSO (3 mL). After stirring at 85° C. for 3 h, the reaction was treated with a 4N HCl solution in dioxane (694 μL, 2.77 mmol) and stirred at 80° C. for 5 h and at RT for 18 h. The reaction was filtered and purified by Xbridge (TFA method) to give the desired product (21 mg, 18%). HRMS-ESI (m/z): [M+H]⁺ calcd for C₆₉H₈₆FN₁₄O₁₂S₃: 1417.5696. found: 1417.5656.

### Example 30: 2-[[6-(1,3-benzothiazol-2-ylamino)-5-methyl-pyridazin-3-yl]-[3-[1-[8-[[2-[2-(2,6-dioxo-3-piperidyl)-1,3-dioxo-isoindolin-4-yl]oxyacetyl]amino]octyl] triazol-4-yl]propyl] amino]-5-[3-[2-fluoro-4-[3-(methylamino)prop-1-ynyl]phenoxy]propyl]thiazole-4-carboxylic acid

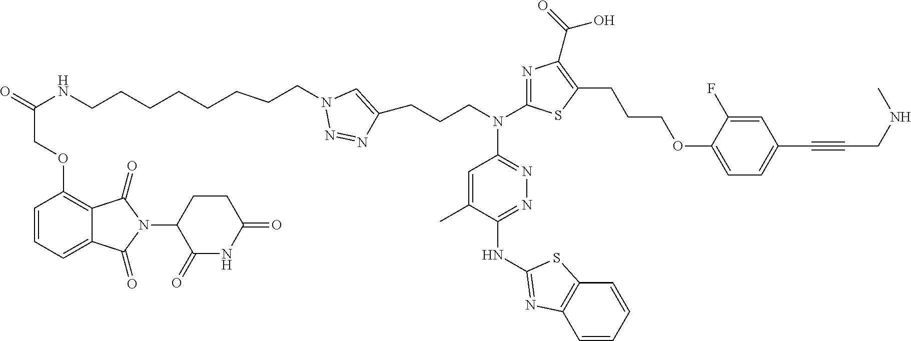 [SMILES: CNCC#Cc1ccc(OCCCc2sc(N(CCCc3cn(CCCCCCCCNC(=O)COc4cccc5c4C(=O)N(C4CCC(=O)NC4=O)C5=O)nn3)c3cc(C)c(Nc4nc5ccccc5s4)nn3)nc2C(=O)O)c(F)c1]

### Step A: N-(8-azidooctyl)-2-[2-(2,6-dioxo-3-piperidyl)-1,3-dioxo-isoindolin-4-yl]oxy-acetamide

To 2-[2-(2,6-dioxo-3-piperidyl)-1,3-dioxo-isoindolin-4-yl]oxyacetic acid (195 mg, 0.139 mL, 0.59 mmol) in DMF (1.5 ml) was added DIEA (102 μL, 0.587 mmol) and TSTU (132.6 mg, 0.441 mmol) and the solution was stirred for 2 h. After the mixture was treated with 8-azidooctan-1-amine [Leenders, Christianus M. A.; et al Chemistry—A European Journal (2016), 22(13), 4608-4615] (100 mg, 0.29 mmol), the reaction was stirred at 80° C. for 5 h. After concentration, the crude product was purified by chromatography on silica by eluting with a gradient [A: DCM] in [B: DCM/MeOH] to give the desired product (100 mg, 70%). ¹H NMR (400 MHz, DMSO-d₆) δ ppm 11.10 (m, 1H), 7.91 (t, 1H), 7.81 (dd, 1H), 7.50 (d, 1H), 7.39 (d, 1H), 5.11 (dd, 1H), 4.76 (s, 2H), 3.31 (t, 2H), 3.14 (q, 2H), 2.89/2.57 (m, 2H), 2.53/2.04 (m, 2H), 1.57-1.20 (m, 12H); IR: 3430-302, 2092, 1770; 1707; 1678.

### Step B: 2-[[6-[(Z)-3H-1,3-benzothiazol-2-ylideneamino]-5-methyl-pyridazin-3-yl]-[3-[1-[8-[[2-[2-(2,6-dioxo-3-piperidyl)-1,3-dioxo-isoindolin-4-yl]oxyacetyl]amino]octyl] triazol-4-yl] propyl]amino]-5-[3-[2-fluoro-4-[3-(methylamino)prop-1-ynyl]phenoxy]propyl]thiazole-4-carboxylic acid

To a solution of CuSO₄×5 H₂O (4.8 mg, 0.027 mmol) in water (1.5 mL) was added THTPA (13.25 mg, 0.030 mmol). This solution was added to a suspension containing the product of Step A (25 mg, 0.052 mmol), the product of Preparation 8 (38 mg, 0.057 mmol), and Na-L-ascorbate (1 mL, 0.5 mmol/L in water, 10 eq) in DMSO (2 mL). The reaction was heated at 85° C. for 1 h. The product was purified by preparative HPLC (Interchim Method) (Xbridge column, TFA method) to give the desired product (11.8 mg, 18%). HRMS-ESI (m/z): [M+H]⁺ calcd for C₅₇H₆₁FN₁₃O₉S₂: 1154.4141. found: 1154.4156.

### Example 31: 2-[3-(1,3-benzothiazol-2-ylamino)-4-methyl-6,7-dihydro-5H-pyrido[2,3-c]pyridazin-8-yl]-5-[3-[2-fluoro-4-[3-[4-[12-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-12-oxo-dodecyl]piperazin-1-yl]prop-1-ynyl]phenoxy]propyl] thiazole-4-carboxylic acid

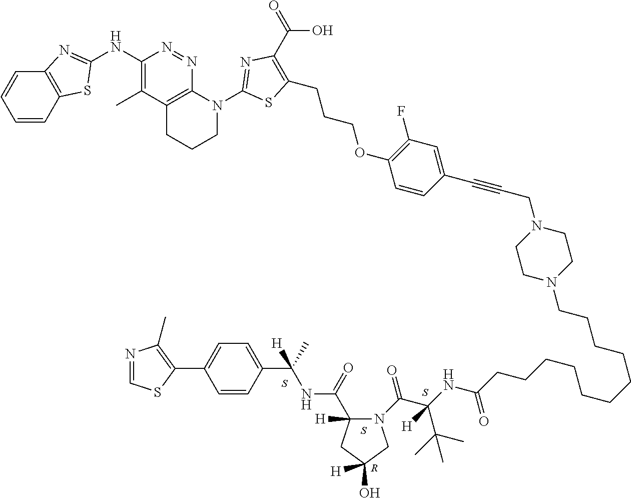

After stirring the product of Preparation 11 (15 mg, 0.018 mmol), the product of Step B of Example 2 (23 mg, 1.6 eq), and DIPEA (0.183 mL, 1.7 eq) in MeCN (0.18 mL) and NMP (0.18 mL) at 70° C. for 18 h, a 10% KOH solution in water (0.051 ml, 5 eq) was added. The reaction was stirred at 40° C. for 4 h. The product was purified by preparative HPLC (Teledyne EZ) (C18, 0.1% aqeuous TFA and MeCN as eluents) to give the desire compound (10 mg, 41%). HRMS-ESI (m/z): [M+2H]²⁺ calcd for C₇₀H₈₉FN₁₂O₇S₃: 662.3062. found: 662.3054.

### Example 32: 2-[[6-(1,3-benzothiazol-2-ylamino)-5-methyl-pyridazin-3-yl]-methyl-amino]-5-[3-[4-[3-[[2-[2-[2-[[2-[2-(2,6-dioxo-3-piperidyl)-1,3-dioxo-isoindolin-4-yl]oxyacetyl]amino]ethoxy]ethylamino]-2-oxo-ethyl]-methyl-amino]prop-1-ynyl]-2-fluoro-phenoxy]propyl]thiazole-4-carboxylic acid

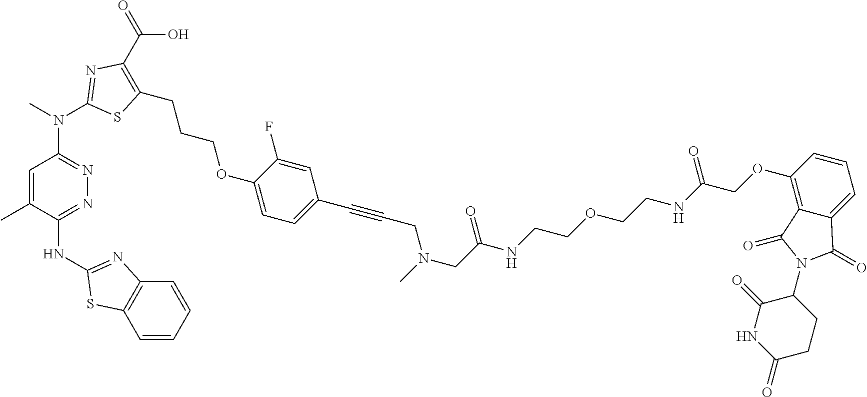 [SMILES: Cc1cc(N(C)c2nc(C(=O)O)c(CCCOc3ccc(C#CCN(C)CC(=O)NCCOCCNC(=O)COc4cccc5c4C(=O)N(C4CCC(=O)NC4=O)C5=O)cc3F)s2)nnc1Nc1nc2ccccc2s1]

### Step A: ethyl 5-(3-chloropropyl)-2-(methylamino)thiazole-4-carboxylate

To the suspension of 2.25 g of methylthiourea (25.0 mmol, 1 eq.) in 100 mL of ethanol was added dropwise 7.46 g of ethyl 3-bromo-6-chloro-2-oxo-hexanoate (1.1 eq.) at 0° C. After 15 min stirring at 0° C., 7 mL of TEA (5.06 g, 2 eq.) was added. After stirring at RT for 18 h and concentration, the residue was portioned between EtOAc and water. The layers were separated, and the organic layer was washed with water and brine, dried, filtered, concentrated and purified by flash column chromatography using heptane and EtOAc as eluents to give 5.0 g (76%) of the desired product. ¹H NMR (400 MHz, DMSO-d₆) δ ppm 7.55 (q, 1H), 4.21 (q, 2H), 3.65 (t, 2H), 3.09 (m, 2H), 2.78 (d, 3H), 1.98 (m, 2H), 1.26 (t, 3H); ¹³C NMR (100 MHz, DMSO-d₆) δ ppm 165.6, 162.5, 137.4, 135.5, 60.5, 45.0, 34.1, 31.2, 24.4, 14.7; HRMS-ESI (m/z): [M+H]⁺ calcd for C₁₀H₁₆ClN₂O₂S: 263.0616. found 263.0615.

### Step B: ethyl 5-(3-chloropropyl)-2-[methyl-[5-methyl-6-[(Z)-[3-(2-trimethylsilyl ethoxy methyl)-1,3-benzothiazol-2-ylidene]amino]pyridazin-3-yl]amino]thiazole-4-carboxylate

The mixture of the product of Preparation 6 (10.2 g, 25 mmol), the product of Step A (1.2 eq.), Pd₂(dba)₃ (851 mg, 0.1 eq), XantPhos (2.9 g, 0.2 eq), Cs₂CO₃ (14.4 g, 3 eq), and DIPEA (9.7 g, 3 eq) in 1,4-dioxane was stirred at 110° C. for 2 h. After concentration, the crude product was purified via flash column chromatography column using heptane and EtOAc as eluents to give 8.81 g (55%) of the desired product. ¹H NMR (500 MHz, DMSO-d₆) δ ppm 7.84 (d, 1H), 7.65 (s, 1H), 7.45 (d, 1H), 7.43 (tm, 1H), 7.25 (tm, 1H), 5.85 (s, 2H), 4.30 (q, 2H), 3.77 (s, 3H), 3.71 (t, 2H), 3.71 (t, 2H), 3.22 (t, 2H), 2.48 (s, 3H), 2.10 (quin, 2H), 1.31 (t, 3H), 0.92 (t, 2H), −0.11 (s, 9H); ¹³C NMR (125 MHz, DMSO-d₆) δ ppm 162.6, 157.4, 156.8, 155.1, 151.7, 140.5, 137.6, 137.1, 135.3, 125.6, 123.5, 123.2, 123.1, 117.6, 111.9, 72.9, 66.7, 60.7, 45.3, 35.4, 34.4, 24.3, 18.0, 17.8, 14.7, −1.0; HRMS-ESI (m/z): [M+H]⁺ calcd for C₂₈H₃₈ClN₆O₃S₂Si: 633.1899. found 633.1891.

### Step C: ethyl 5-(3-iodopropyl)-2-[methyl-[5-methyl-6-[(Z)-[3-(2-trimethylsilylethoxy methyl)-1,3-benzothiazol-2-ylidene]amino]pyridazin-3-yl]amino]thiazole-4-carboxylate

The mixture of 2.6 g of the product from Step B (4.1 mmol, 1 eq.), 1.23 g of NaI (2 eq.) in 20 mL of acetone was stirred at 60° C. for 3 days. The reaction mixture was diluted with water and the precipitated product was filtered off, washed with water, and dried to give 2.5 g (84%) of the desired product. ¹H NMR (500 MHz, DMSO-d₆) δ 7.82 (d, 1H), 7.61 (s, 1H), 7.47-7.39 (m, 1H), 7.47-7.39 (m, 1H), 7.23 (t, 1H), 5.83 (s, 2H), 4.29 (q, 2H), 3.75 (s, 3H), 3.71 (t, 2H), 3.33 (t, 2H), 3.16 (t, 2H), 2.42 (s, 3H), 2.13 (quint., 2H), 1.33 (t, 3H), 0.91 (t, 2H), −0.12 (s, 9H); ¹³C NMR (125 MHz, DMSO-d₆) δ ppm 162.6, 157.3, 156.7, 155.1, 151.6, 140.2, 137.6, 137.1, 135.2, 127.1, 125.4, 123.4, 123.2, 117.5, 111.9, 72.8, 66.7, 60.7, 35.2, 35.2, 27.6, 17.8, 17.8, 14.8, 7.8, −1.0; HRMS-ESI (m/z): [M+H]⁺ calcd for C₂₈H₃₈IN₆O₃S₂Si: 725.1255. found 725.1248.

### Step D: ethyl 5-[3-(2-fluoro-4-iodo-phenoxy)propyl]-2-[methyl-[5-methyl-6-[(Z)-[3-(2-trimethylsilylethoxymethyl)-1,3-benzothiazol-2-ylidene]amino]pyridazin-3-yl]amino]thiazole-4-carboxylate

To the mixture of the product of Step C (3.62 g, 5.0 mmol) and Cs₂CO₃ (3.25 g, 2 eq) in acetone (25 mL) was added 2-fluoro-4-iodo-phenol (1.20 g, 1 eq). The reaction was stirred for 3 h. After concentration, the product was purified by flash column chromatography column using heptane and EtOAc as eluents to give 3.0 g (72%) of the desired product. ¹H NMR (500 MHz, dmso-d6) δ ppm 7.81 (dm, 1H), 7.58 (s, 1H), 7.58 (dd, 1H), 7.43 (m, 1H), 7.42 (m, 1H), 7.41 (m, 1H), 7.23 (m, 1H), 6.97 (t, 1H), 5.81 (s, 2H), 4.24 (q, 2H), 4.08 (t, 2H), 3.74 (s, 3H), 3.70 (m, 2H), 3.22 (t, 2H), 2.41 (s, 3H), 2.09 (m, 2H), 1.28 (t, 3H), 0.9 (m, 2H), −0.12 (s, 9H); ¹³C NMR (125 MHz, dmso-d6) δ ppm 134.0, 127.1, 124.8, 123.4, 123.1, 117.7, 117.5, 111.8, 72.9, 68.5, 66.7, 60.7, 35.2, 31.0, 23.3, 17.8, 17.7, 14.7, −1.0.

### Step E: 5-[3-(2-fluoro-4-iodo-phenoxy)propyl]-2-[methyl-[5-methyl-6-[(Z)-[3-(2-trimethyl silylethoxymethyl)-1,3-benzothiazol-2-ylidene]amino]pyridazin-3-yl]amino]thiazole-4-carboxylic acid

The mixture of the product of Step D (2.0 g, 2.4 mmol) and LiOH*H₂O (1.0 g, 10 eq) was stirred in a mixture of 1,4-dioxane (10 mL), EtOH (75 mL), and water (22 mL) at 80° C. for 30 min. After setting the pH to 4 using a 1 M solution of HCl, the desired product was filtered off (1.49 g, 77%). ¹H NMR (500 MHz, dmso-d6) δ ppm 7.84 (d, 1H), 7.64 (s, 1H), 7.58 (dd, 1H), 7.44 (d, 1H), 7.44 (t, 1H), 7.44 (dd, 1H), 7.25 (t, 1H), 6.99 (t, 1H), 5.84 (s, 2H), 4.08 (t, 2H), 3.71 (t, 2H), 3.67 (s, 3H), 3.30 (t, 2H), 2.43 (s, 3H), 2.09 (qn, 2H), 0.91 (t, 2H), −0.12 (s, 9H); ¹³C NMR (125 MHz, dmso-d6) δ ppm 134.1, 127.1, 124.9, 123.4, 123.2, 118.0, 117.7, 111.9, 72.8, 69.0, 66.7, 35.8, 31.3, 22.7, 17.8, 17.8, −0.9.

### Step F: (4-methoxyphenyl)methyl 5-[3-(2-fluoro-4-iodo-phenoxy)propyl]-2-[methyl-[5-methyl-6-[(Z)-[3-(2-trimethylsilylethoxymethyl)-1,3-benzothiazol-2-ylidene]amino]pyridazin-3-yl]amino]thiazole-4-carboxylate

To the mixture of the product of Step E (1.49 g, 1.84 mmol) and Cs₂CO₃ (1.20 g, 2 eq) in DMF (10 ml) was added 1-(chloromethyl)-4-methoxy-benzene (0.28 mL, 1.1 eq). The reaction was stirred at 70° C. for 18 h. After dilution with water, the mixture was extracted with EtOAc. The organic phases were dried, concentrated and purified by flash column chromatography column using heptane and EtOAc as eluents to give 612 mg of the desired product.

### Step G: (4-methoxyphenyl)methyl 5-[3-[4-[3-[[2-[2-[2-[[2-[2-(2,6-dioxo-3-piperidyl)-1,3-dioxo-isoindolin-4-yl]oxyacetyl]amino]ethoxy]ethylamino]-2-oxo-ethyl]-methyl-amino]prop-1-ynyl]-2-fluoro-phenoxy]propyl]-2-[methyl-[5-methyl-6-[(E)-[3-(2-trimethylsilyl ethoxymethyl)-1,3-benzothiazol-2-ylidene]amino]pyridazin-3-yl]amino]thiazole-4-carboxylate

To the mixture of the product of Step F (215 mg, 0.23 mmol), Pd(PPh₃)₂Cl₂ (33 mg, 0.2 eq), and CuI (9 mg, 0.2 eq) in DIPA (9 mL) was added the product of Preparation 12 (245 mg, 2 eq) in MeCN (3 mL). The reaction was stirred at 70° C. for 2.5 h. The product was purified by flash column chromatography column using DCM and MeOH as eluents to give 187 mg (60%) of the desired product.

### Step H: 2-[[6-(1,3-benzothiazol-2-ylamino)-5-methyl-pyridazin-3-yl]-methyl-amino]-5-[3-[4-[3-[[2-[2-[2-[[2-[2-(2,6-dioxo-3-piperidyl)-1,3-dioxo-isoindolin-4-yl]oxyacetyl] amino]ethoxy]ethylamino]-2-oxo-ethyl]-methyl-amino]prop-1-ynyl]-2-fluoro-phenoxy]propyl]thiazole-4-carboxylic acid

After stirring the product of Step G (187 mg, 0.14 mmol) in MeCN (5 mL) and TFA (5 ml) for 36 h, the product was purified by preparative HPLC (Teledyne EZ) (C18, 0.2% aqeuous HCOOH, MeCN) to give the desired product (59 mg, 40%). ¹H NMR (500 MHz, dmso-d6) δ ppm 11.12 (s, 1H), 8.00 (t, 1H), 7.90 (br., 1H), 7.80 (dd, 1H), 7.67 (brs., 1H), 7.53 (br., 1H), 7.49 (d, 1H), 7.38 (m, 1H), 7.38 (t, 1H), 7.38 (d, 1H), 7.26 (brd., 1H), 7.21 (t, 1H), 7.18 (br., 1H), 5.12 (dd, 1H), 4.78 (s, 2H), 4.15 (t, 2H), 3.94/3.40 (br+br., 4H), 3.77 (s, 3H), 3.46 (t, 2H), 3.45 (t, 2H), 3.32 (q, 2H), 3.30 (q, 2H), 3.28 (t, 2H), 2.89/2.59 (m+m, 2H), 2.56 (br., 3H), 2.56/2.09 (m+m, 2H), 2.46 (s, 3H), 2.14 (m, 2H); ¹³C NMR (125 MHz, dmso-d6) δ ppm 173.3, 170.4, 167.4, 167.2, 166.0, 164.1, 156.6, 155.4, 151.9, 137.4, 129.3, 126.6, 122.6, 122.3, 120.8, 119.5, 118.4, 116.5, 115.5, 69.0, 69.0, 68.6, 67.9, 49.3, 38.9, 38.7, 35.3, 31.4, 30.9, 23.1, 22.5, 17.8; IR: 2946, 1709, 1668, 1614; HRMS-ESI (m/z): [M+Na]⁺ calcd for C₅₁H₅₀FN₁₁NaO₁₁S₂: 1098.3014. found 1098.3006.

### Example 33: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(8-(((S)-1-((2S,4R)-4-hydroxy-2-((4-(4-methyl thiazol-5-yl)benzyl)carbamoyl)pyrrolidin-1-yl)-3,3-dimethyl-1-oxobutan-2-yl)amino)-8-oxo octanoyl)piperazin-1-yl)prop-1-yn-1-yl)phenoxy)propyl) thiazole-4-carboxylic acid

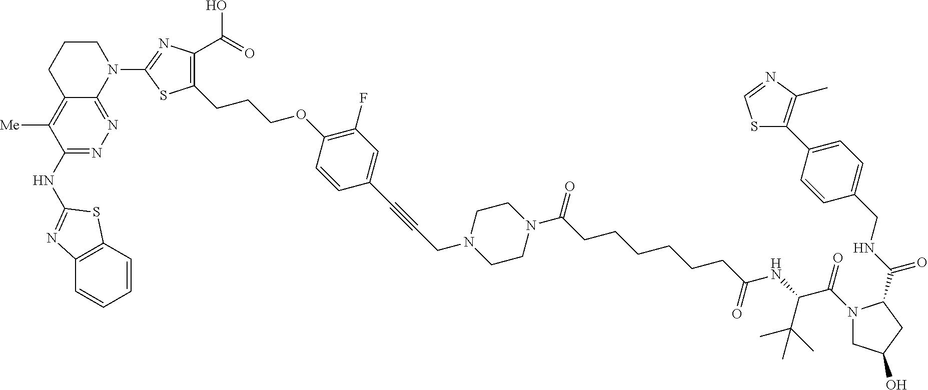 [SMILES: Cc1ncsc1-c1ccc(CNC(=O)[C@@H]2C[C@@H](O)CN2C(=O)[C@@H](NC(=O)CCCCCCC(=O)N2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1]

### General Procedure 4:

To 8-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-8-oxooctanoic acid (27.3 mg, 0.047 mmoles) in DMF (0.75 mL) was added HATU (17.7 mg, 0.047 mmoles) and DIPEA (24.9 uL, 0.143 mmoles). After stirring for 15 minutes, to the activated acid solution was added 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl)phenoxy)propyl)thiazole-4-carboxylic acid (25 mg, 0.036 mmoles). The mixture was stirred for 4 hours at RT. DMSO (2.5 mL) was added and the solution was purified by RP-HPLC ISCO gold chromatography (10-100% MeCN/H2O, 0.1% NH4OH modifier). Upon lyophilization, 2-(3-(benzo[d]thiazol-2-ylamino)-4-methyl-6,7-dihydropyrido[2,3-c]pyridazin-8(5H)-yl)-5-(3-(2-fluoro-4-(3-(4-(8-(((S)-1-((2S,4R)-4-hydroxy-2-((4-(4-methylthiazol-5-yl) benzyl)carbamoyl)pyrrolidin-1-yl)-3,3-dimethyl-1-oxobutan-2-yl)amino)-8-oxooctanoyl) piperazin-1-yl)prop-1-yn-1-yl)phenoxy)propyl)thiazole-4-carboxylic acid (13.0 mg, 0.0097 mmoles) was obtained as the ammonium salt. HRMS: MH+=1267.4900; Rt=2.20 min (5 min acidic method).

Note, in cases where the BCLxL amine starting material contained a PMB ester or NHBoc moiety the product after amide coupling (isolated either by RP chromatography and lyophilization or by preciptating from H2O, dissolving in MeCN/H2O and lyophilizing) could be deprotected by treating with 25% trifluoroacetic acid in dichloromethane for 1 hour followed by removal of volatiles and purification by RP-HPLC ISCO gold chromatography (10-100% MeCN/H2O, 0.1% NH4OH modifier).

### Example 34: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl) amino)-7-oxoheptanoyl)piperazin-1-yl)prop-1-yn-1-yl)phenoxy)propyl)thiazole-4-carboxylic acid

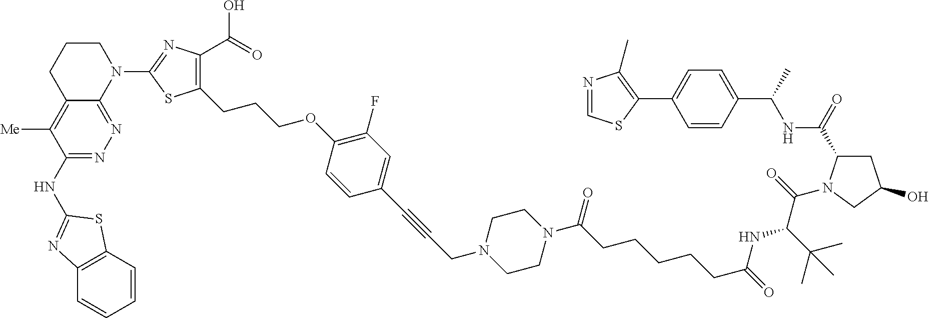 [SMILES: Cc1ncsc1-c1ccc([C@H](C)NC(=O)[C@@H]2C[C@@H](O)CN2C(=O)[C@@H](NC(=O)CCCCCC(=O)N2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1]

Following General Procedure 4, using 7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoic acid (25.2 mg, 0.043 mmoles) and 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl)phenoxy)propyl)thiazole-4-carboxylic acid (25 mg, 0.036 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoyl)piperazin-1-yl)prop-1-yn-1-yl)phenoxy)propyl)thiazole-4-carboxylic acid was obtained as the ammonium salt. HRMS: MH+=1267.4900; Rt=2.23 min (5 min acidic method).

### Example 35: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl) amino)-5-oxopentanoyl)piperazin-1-yl)prop-1-yn-1-yl)phenoxy)propyl)thiazole-4-carboxylic acid

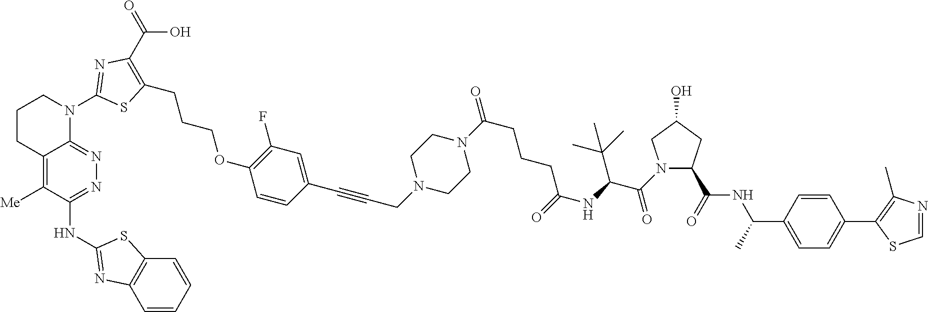 [SMILES: Cc1ncsc1-c1ccc([C@H](C)NC(=O)[C@@H]2C[C@@H](O)CN2C(=O)[C@@H](NC(=O)CCCC(=O)N2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1]

Following General Procedure 4, using 5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoic acid (12.0 mg, 0.021 mmoles) and 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl)phenoxy)propyl)thiazole-4-carboxylic acid (15.0 mg, 0.021 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoyl)piperazin-1-yl)prop-1-yn-1-yl)phenoxy)propyl)thiazole-4-carboxylic acid was obtained as the ammonium salt. HRMS: MH+=1239.4100; Rt=2.18 min (5 min acidic method).

### Example 36: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(N-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)-N-methylglycyl)piperazin-1-yl)prop-1-yn-1-yl)phenoxy)propyl) thiazole-4-carboxylic acid

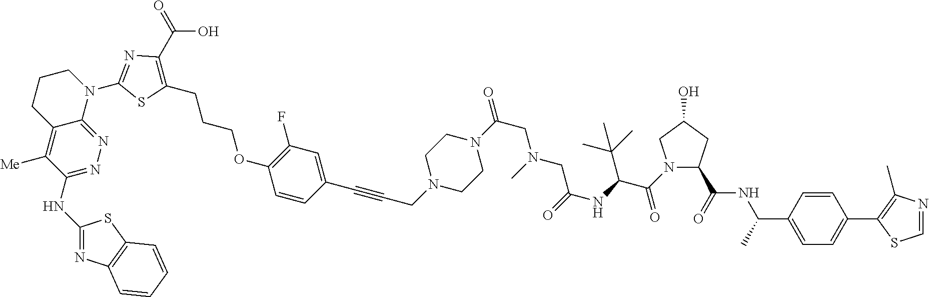 [SMILES: Cc1ncsc1-c1ccc([C@H](C)NC(=O)[C@@H]2C[C@@H](O)CN2C(=O)[C@@H](NC(=O)CN(C)CC(=O)N2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1]

Following General Procedure 4, using N-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)-N-methylglycine (22 mg, 0.032 mmoles) and 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl)phenoxy)propyl)thiazole-4-carboxylic acid (20 mg, 0.029 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(N-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)-N-methylglycyl)piperazin-1-yl)prop-1-yn-1-yl)phenoxy)propyl)thiazole-4-carboxylic acid was obtained as the ammonium salt. HRMS: MH+=1254.4700; Rt=2.01 min (5 min acidic method).

### Example 37: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(2-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)thio)acetyl)piperazin-1-yl)prop-1-yn-1-yl)phenoxy)propyl)thiazole-4-carboxylic acid

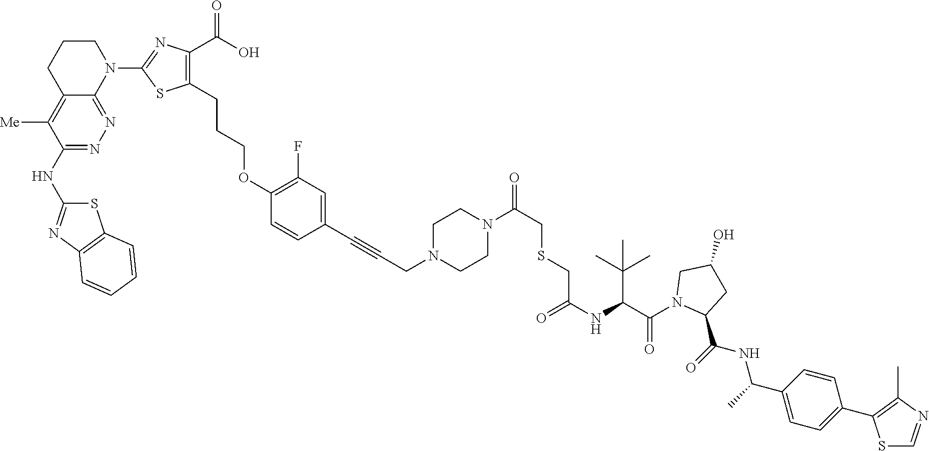 [SMILES: Cc1ncsc1-c1ccc([C@H](C)NC(=O)[C@@H]2C[C@@H](O)CN2C(=O)[C@@H](NC(=O)CSCC(=O)N2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1]

Following General Procedure 4 using 2-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)thio)acetic acid (16.3 mg, 0.024 mmoles) and 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl)phenoxy)propyl)thiazole-4-carboxylic acid (15 mg, 0.021 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(2-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)thio)acetyl)piperazin-1-yl)prop-1-yn-1-yl)phenoxy)propyl)thiazole-4-carboxylic acid was obtained as the ammonium salt. HRMS: MH+=xx; Rt=xx min (5 min acidic method).

### Example 38: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl) amino)-9-oxononanoyl)piperazin-1-yl)prop-1-yn-1-yl)phenoxy)propyl)thiazole-4-carboxylic acid

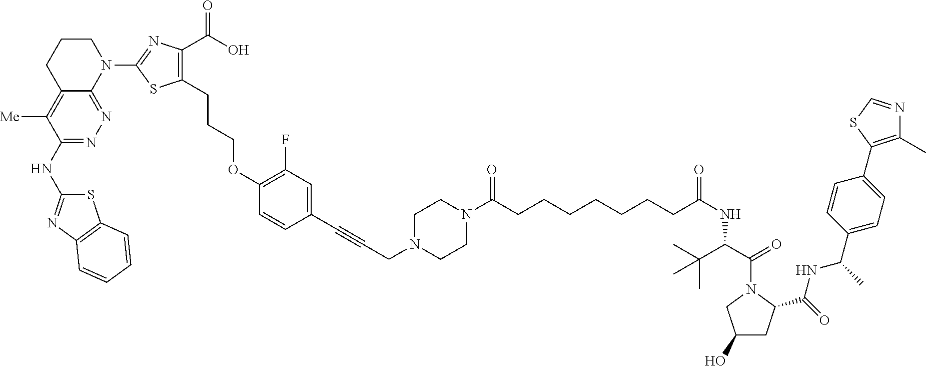 [SMILES: Cc1ncsc1-c1ccc([C@H](C)NC(=O)[C@@H]2C[C@@H](O)CN2C(=O)[C@@H](NC(=O)CCCCCCCC(=O)N2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1]

Following General Procedure 4, using 9-(((S)-1-((2S,4R)-4-hydroxy-2-(((S)-1-(4-(4-methyl thiazol-5-yl)phenyl)ethyl)carbamoyl)pyrrolidin-1-yl)-3,3-dimethyl-1-oxobutan-2-yl)amino)-9-oxononanoic acid (15.8 mg, 0.026 mmoles) and 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl) phenoxy)propyl)thiazole-4-carboxylic acid (15.0 mg, 0.021 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoyl)piperazin-1-yl)prop-1-yn-1-yl)phenoxy) propyl)thiazole-4-carboxylic acid was obtained as the ammonium salt. HRMS: MH+=1295.5300; Rt=2.32 min (5 min acidic method).

### Example 39: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(2-(1-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclopentyl)acetyl)piperazin-1-yl)prop-1-yn-1-yl)phenoxy)propyl) thiazole-4-carboxylic acid

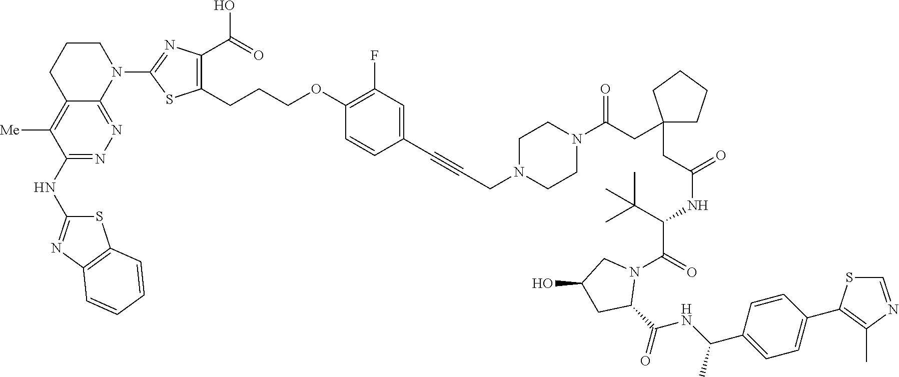 [SMILES: Cc1ncsc1-c1ccc([C@H](C)NC(=O)[C@@H]2C[C@@H](O)CN2C(=O)[C@@H](NC(=O)CC2(CC(=O)N3CCN(CC#Cc4ccc(OCCCc5sc(N6CCCc7c6nnc(Nc6nc8ccccc8s6)c7C)nc5C(=O)O)c(F)c4)CC3)CCCC2)C(C)(C)C)cc1]

Following General Procedure 4, using 2-(1-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclopentyl)acetic acid (15.2 mg, 0.025 mmoles) and 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl)phenoxy)propyl)thiazole-4-carboxylic acid (14.5 mg, 0.021 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(2-(1-(2-(((S)-1-((2S,4R)-4-hydroxy-2-(((S)-1-(4-(4-methylthiazol-5-yl)phenyl)ethyl) carbamoyl)pyrrolidin-1-yl)-3,3-dimethyl-1-oxobutan-2-yl)amino)-2-oxoethyl)cyclopentyl) acetyl)piperazin-1-yl)prop-1-yn-1-yl)phenoxy)propyl)thiazole-4-carboxylic acid was obtained as the ammonium salt. HRMS: MH+=1293.5100; Rt=2.38 min (5 min acidic method).

### Example 40: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-((S)-13-((2S,4R)-4-hydroxy-2-((4-(4-methyl thiazol-5-yl)benzyl)carbamoyl)pyrrolidine-1-carbonyl)-14,14-dimethyl-11-oxo-3,6,9-trioxa-12-azapentadecanoyl)piperazin-1-yl)prop-1-yn-1-yl)phenoxy)propyl)thiazole-4-carboxylic acid

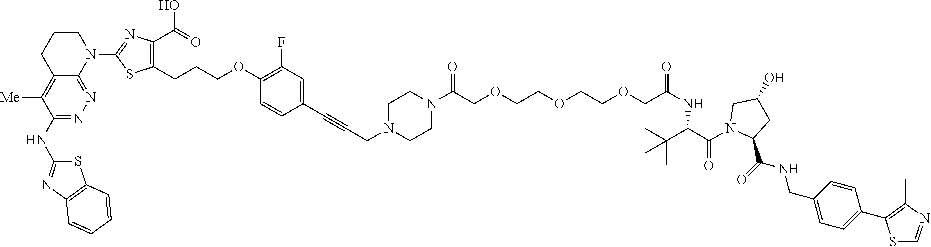 [SMILES: Cc1ncsc1-c1ccc(CNC(=O)[C@@H]2C[C@@H](O)CN2C(=O)[C@@H](NC(=O)COCCOCCOCC(=O)N2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1]

Following General Procedure 4, using (S)-13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapenta decanoic acid (16.4 mg, 0.026 mmoles) and 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl) phenoxy)propyl)thiazole-4-carboxylic acid (15.0 mg, 0.021 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-((S)-13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecanoyl)piperazin-1-yl)prop-1-yn-1-yl)phenoxy)propyl)thiazole-4-carboxylic acid was obtained as the ammonium salt. HRMS: MH+=1315.4800; Rt=2.17 min (5 min acidic method).

### Example 41: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl) carbamoyl)spiro[3.3]heptane-2-carbonyl)piperazin-1-yl)prop-1-yn-1-yl)phenoxy)propyl) thiazole-4-carboxylic acid

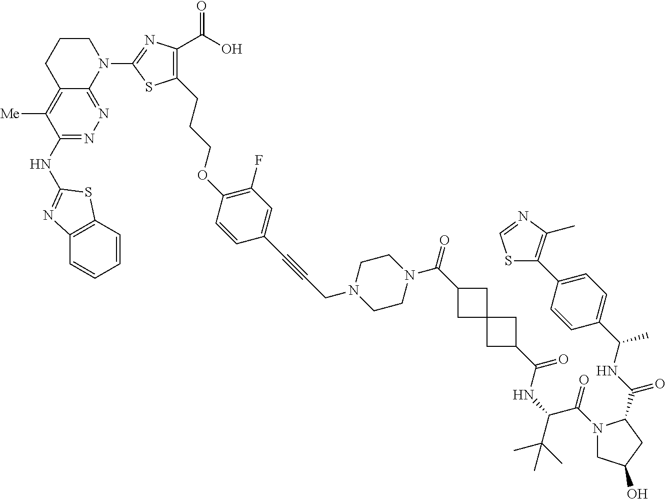 [SMILES: Cc1ncsc1-c1ccc([C@H](C)NC(=O)[C@@H]2C[C@@H](O)CN2C(=O)[C@@H](NC(=O)C2CC3(C2)CC(C(=O)N2CCN(CC#Cc4ccc(OCCCc5sc(N6CCCc7c6nnc(Nc6nc8ccccc8s6)c7C)nc5C(=O)O)c(F)c4)CC2)C3)C(C)(C)C)cc1]

Following General Procedure 4 using 6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl) carbamoyl)spiro[3.3]heptane-2-carboxylic acid (15.7 mg, 0.026 mmoles) and 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl)phenoxy)propyl)thiazole-4-carboxylic acid (15.0 mg, 0.021 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(6-(((S)-1-((2S,4R)-4-hydroxy-2-(((S)-1-(4-(4-methylthiazol-5-yl)phenyl) ethyl)carbamoyl)pyrrolidin-1-yl)-3,3-dimethyl-1-oxobutan-2-yl)carbamoyl)spiro[3.3]heptane-2-carbonyl)piperazin-1-yl)prop-1-yn-1-yl)phenoxy)propyl)thiazole-4-carboxylic acid was obtained as the ammonium salt. HRMS: MH+=1291.4900; Rt=2.28 min (5 min acidic method).

### Example 42: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(2-((3S,5R)-3-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)adamantan-1-yl)acetyl)piperazin-1-yl)prop-1-yn-1-yl) phenoxy)propyl)thiazole-4-carboxylic acid

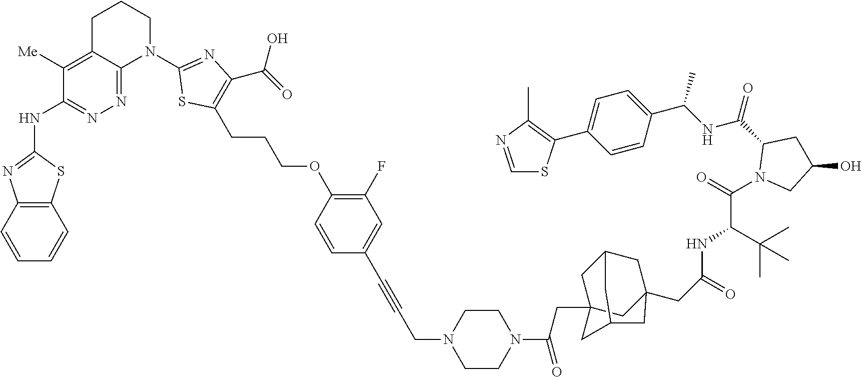 [SMILES: Cc1ncsc1-c1ccc([C@H](C)NC(=O)[C@@H]2C[C@@H](O)CN2C(=O)[C@@H](NC(=O)CC23CC4CC(C2)CC(CC(=O)N2CCN(CC#Cc5ccc(OCCCc6sc(N7CCCc8c7nnc(Nc7nc9ccccc9s7)c8C)nc6C(=O)O)c(F)c5)CC2)(C4)C3)C(C)(C)C)cc1]

Following General Procedure 4, using 2-((3S,5R)-3-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)adamantan-1-yl)acetic acid (14.57 mg, 0.021 mmoles) and 2-(3-(benzo[d] thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl)phenoxy)propyl)thiazole-4-carboxylic acid (15.0 mg, 0.021 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(2-((3S,5R)-3-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl) adamantan-1-yl)acetyl)piperazin-1-yl)prop-1-yn-1-yl)phenoxy)propyl)thiazole-4-carboxylic acid was obtained as the ammonium salt. HRMS: MH+=1359.5600; Rt=2.45 min (5 min acidic method).

### Example 43: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(3-fluoro-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)benzoyl)piperazin-1-yl)prop-1-yn-1-yl)phenoxy)propyl)thiazole-4-carboxylic acid

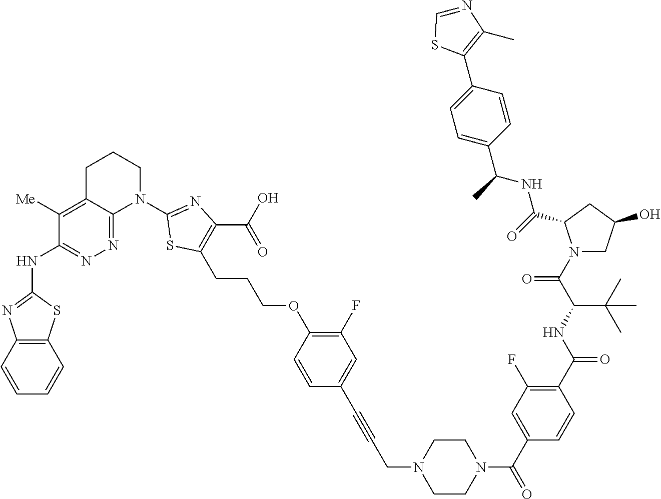 [SMILES: Cc1ncsc1-c1ccc([C@H](C)NC(=O)[C@@H]2C[C@@H](O)CN2C(=O)[C@@H](NC(=O)c2ccc(C(=O)N3CCN(CC#Cc4ccc(OCCCc5sc(N6CCCc7c6nnc(Nc6nc8ccccc8s6)c7C)nc5C(=O)O)c(F)c4)CC3)cc2F)C(C)(C)C)cc1]

Following General Procedure 4 using 3-fluoro-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl) carbamoyl)benzoic acid (15.7 mg, 0.026 mmoles) and 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl)phenoxy)propyl)thiazole-4-carboxylic acid (15.0 mg, 0.021 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(3-fluoro-4-(((S)-1-((2S,4R)-4-hydroxy-2-(((S)-1-(4-(4-methylthiazol-5-yl)phenyl)ethyl)carbamoyl) pyrrolidin-1-yl)-3,3-dimethyl-1-oxobutan-2-yl)carbamoyl)benzoyl)piperazin-1-yl)prop-1-yn-1-yl)phenoxy)propyl)thiazole-4-carboxylic acid was obtained as the ammonium salt. HRMS: MH+=1291.4399; Rt=2.34 min (5 min acidic method).

### Example 44: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(5-(2-(2-(((2S,4R)-1-((S)-2-(1-fluorocyclo propane-1-carboxamido)-3,3-dimethylbutanoyl)-4-hydroxypyrrolidine-2-carboxamido) methyl)-5-(4-methylthiazol-5-yl)phenoxy)acetamido)pentanoyl)piperazin-1-yl)prop-1-yn-1-yl)phenoxy)propyl)thiazole-4-carboxylic acid

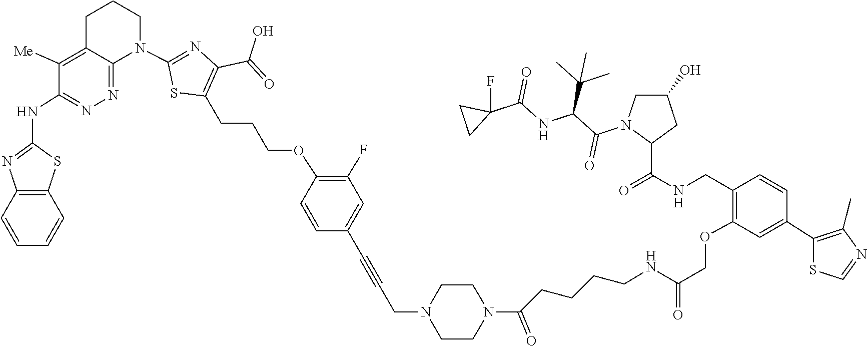 [SMILES: Cc1ncsc1-c1ccc(CNC(=O)C2C[C@@H](O)CN2C(=O)[C@@H](NC(=O)C2(F)CC2)C(C)(C)C)c(OCC(=O)NCCCCC(=O)N2CCN(CC#Cc3ccc(OCCCc4sc(N5CCCc6c5nnc(Nc5nc7ccccc7s5)c6C)nc4C(=O)O)c(F)c3)CC2)c1]

Following General Procedure 4, using 5-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methyl thiazol-5-yl)phenoxy)acetamido)pentanoic acid (11.9 mg, 0.017 mmoles) and 2-(3-(benzo[d] thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl)phenoxy)propyl)thiazole-4-carboxylic acid (11.0 mg, 0.016 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(5-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy) acetamido)pentanoyl)piperazin-1-yl)prop-1-yn-1-yl)phenoxy)propyl)thiazole-4-carboxylic acid was obtained as the ammonium salt. HRMS: MH+=1370.5000; Rt=2.26 min (5 min acidic method).

### Example 45: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(12-(((S)-1-((2S,4R)-4-hydroxy-2-((2-hydroxy-4-(4-methylthiazol-5-yl)benzyl)carbamoyl)pyrrolidin-1-yl)-3,3-dimethyl-1-oxobutan-2-yl)amino)-12-oxododecanoyl)piperazin-1-yl)prop-1-yn-1-yl)phenoxy) propyl)thiazole-4-carboxylic acid

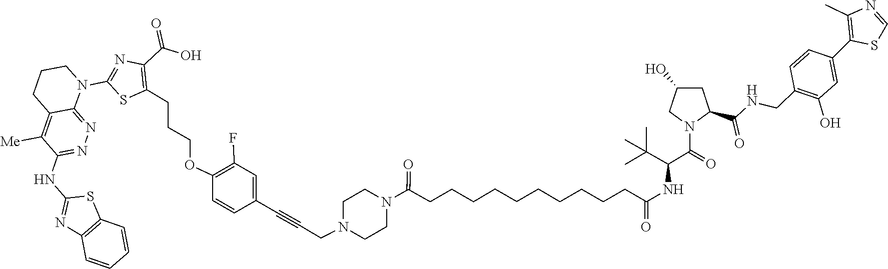 [SMILES: Cc1ncsc1-c1ccc(CNC(=O)[C@@H]2C[C@@H](O)CN2C(=O)[C@@H](NC(=O)CCCCCCCCCCC(=O)N2CCN(CC#Cc3ccc(OCCCc4sc(N5CCCc6c5nnc(Nc5nc7ccccc7s5)c6C)nc4C(=O)O)c(F)c3)CC2)C(C)(C)C)c(O)c1]

Following General Procedure 4, using 12-(((S)-1-((2S,4R)-4-hydroxy-2-((2-hydroxy-4-(4-methylthiazol-5-yl)benzyl)carbamoyl)pyrrolidin-1-yl)-3,3-dimethyl-1-oxobutan-2-yl)amino)-12-oxododecanoic acid (15.6 mg, 0.024 mmoles) and 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl)phenoxy)propyl)thiazole-4-carboxylic acid (15.0 mg, 0.021 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(12-(((S)-1-((2S,4R)-4-hydroxy-2-((2-hydroxy-4-(4-methylthiazol-5-yl)benzyl)carbamoyl)pyrrolidin-1-yl)-3,3-dimethyl-1-oxobutan-2-yl)amino)-12-oxododecanoyl)piperazin-1-yl)prop-1-yn-1-yl)phenoxy) propyl) thiazole-4-carboxylic acid was obtained as the ammonium salt. HRMS: MH+=1339.5699; Rt=2.31 min (5 min acidic method).

### Example 46: Synthesis of 2-(3-(benzo[d]thiazol-2-ylamino)-4-methyl-6,7-dihydro pyrido [2,3-c]pyridazin-8(5H)-yl)-5-(3-(2-fluoro-4-(3-(4-(12-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-12-oxododecanoyl)piperazin-1-yl)prop-1-yn-1-yl)phenoxy)propyl)thiazole-4-carboxylic acid

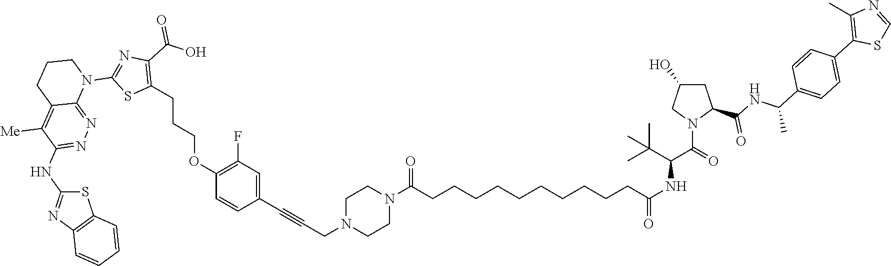 [SMILES: Cc1ncsc1-c1ccc([C@H](C)NC(=O)[C@@H]2C[C@@H](O)CN2C(=O)[C@@H](NC(=O)CCCCCCCCCCC(=O)N2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1]

Following General Procedure 4, using 12-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-12-oxododecanoic acid (21.4 mg, 0.033 mmoles) and 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl)phenoxy)propyl)thiazole-4-carboxylic acid (19.0 mg, 0.027 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(12-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-12-oxododecanoyl)piperazin-1-yl)prop-1-yn-1-yl)phenoxy) propyl)thiazole-4-carboxylic acid was obtained as the ammonium salt. HRMS: MH+=1337.5900; Rt=2.39 min (5 min acidic method).

### Example 47: Synthesis of 6-(3-(benzo[d]thiazol-2-ylamino)-4-methyl-6,7-dihydropyrido [2,3-c]pyridazin-8(5H)-yl)-3-(1-(((1r,3s,5R,7S)-3-(2-(4-(12-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxo butan-2-yl)amino)-12-oxododecanoyl)piperazin-1-yl)ethoxy)-5,7-dimethyladamantan-1-yl)methyl)-5-methyl-1H-pyrazol-4-yl)picolinic acid

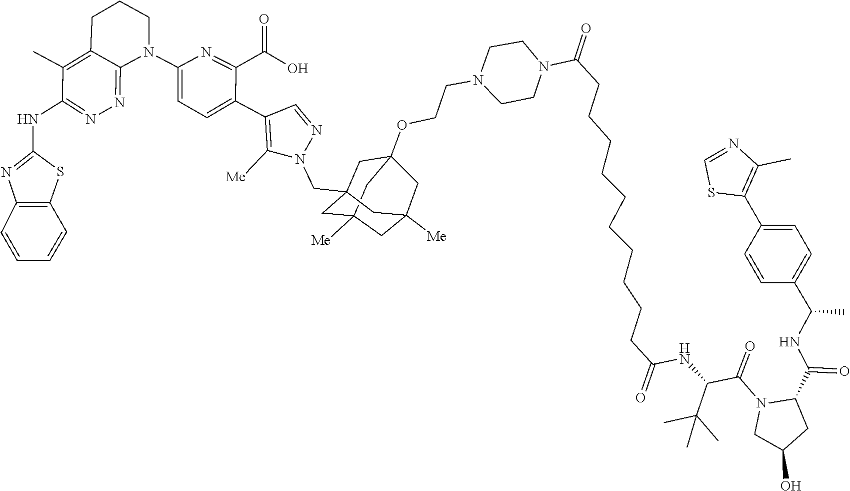 [SMILES: Cc1ncsc1-c1ccc([C@H](C)NC(=O)[C@@H]2C[C@@H](O)CN2C(=O)[C@@H](NC(=O)CCCCCCCCCCC(=O)N2CCN(CCOC34CC5(C)CC(C)(CC(Cn6ncc(-c7ccc(N8CCCc9c8nnc(Nc8nc%10ccccc%10s8)c9C)nc7C(=O)O)c6C)(C5)C3)C4)CC2)C(C)(C)C)cc1]

Following General Procedure 4, using 12-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-12-oxododecanoic acid (6.9 mg, 0.0106 mmoles) and 6-(3-(benzo[d]thiazol-2-ylamino)-4-methyl-6,7-dihydropyrido[2,3-c]pyridazin-8(5H)-yl)-3-(1-(((1 r,3R,5S,7s)-3,5-dimethyl-7-(2-(piperazin-1-yl)ethoxy)adamantan-1-yl)methyl)-5-methyl-1H-pyrazol-4-yl)picolinic acid (7.7 mg, 0.0096 mmoles), 6-(3-(benzo[d]thiazol-2-ylamino)-4-methyl-6,7-dihydropyrido[2,3-c]pyridazin-8(5H)-yl)-3-(1-(((1 r,3s,5R,7S)-3-(2-(4-(12-(((S)-1-((2S,4R)-4-hydroxy-2-(((S)-1-(4-(4-methyl thiazol-5-yl)phenyl)ethyl)carbamoyl)pyrrolidin-1-yl)-3,3-dimethyl-1-oxobutan-2-yl)amino)-12-oxododecanoyl)piperazin-1-yl)ethoxy)-5,7-dimethyladamantan-1-yl)methyl)-5-methyl-1H-pyrazol-4-yl)picolinic acid was obtained as the ammonium salt. HRMS: MH+=1441.7700; Rt=2.30 min (5 min acidic method).

### Example 48: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(12-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-12-oxododecanoyl)piperazin-1-yl)prop-1-yn-1-yl)phenoxy)propyl)thiazole-4-carboxylic acid

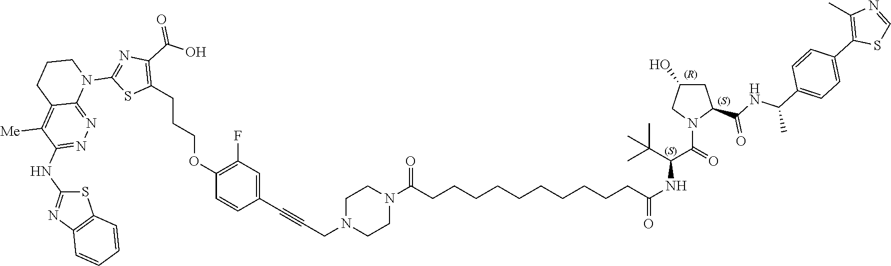

Following General Procedure 4, using 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl) phenoxy)propyl)thiazole-4-carboxylic acid (12 mg, 0.17 mmoles) and 12-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-12-oxododecanoic acid (13.25 mg, 0.021 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(12-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-12-oxododecanoyl)piperazin-1-yl)prop-1-yn-1-yl)phenoxy)propyl)thiazole-4-carboxylic acid was obtained as the ammonium salt. HRMS: MH+=1323.5400; Rt=2.33 min (5 min acidic method).

### Example 49: Synthesis of 6-(3-(benzo[d]thiazol-2-ylamino)-4-methyl-6,7-dihydropyrido [2,3-c]pyridazin-8(5H)-yl)-3-(1-(((1r,3s,5R,7S)-3-(2-(4-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutanoyl)piperazin-1-yl)ethoxy)-5,7-dimethyladamantan-1-yl)methyl)-5-methyl-1H-pyrazol-4-yl)picolinic acid

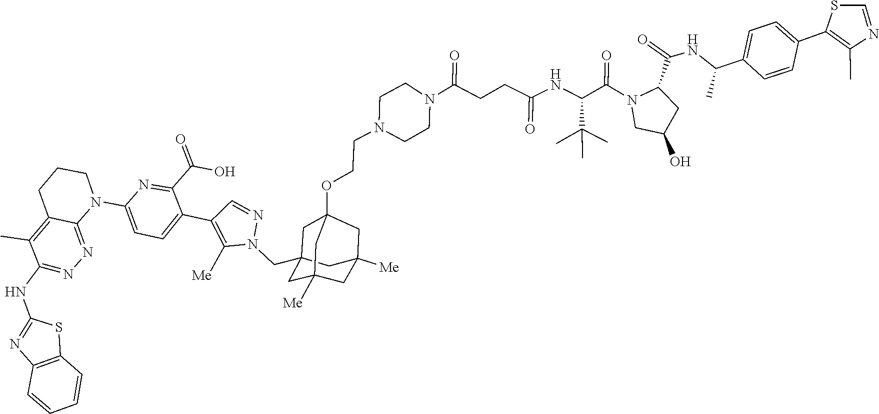 [SMILES: Cc1ncsc1-c1ccc([C@H](C)NC(=O)[C@@H]2C[C@@H](O)CN2C(=O)[C@@H](NC(=O)CCC(=O)N2CCN(CCOC34CC5(C)CC(C)(CC(Cn6ncc(-c7ccc(N8CCCc9c8nnc(Nc8nc%10ccccc%10s8)c9C)nc7C(=O)O)c6C)(C5)C3)C4)CC2)C(C)(C)C)cc1]

Following General Procedure 4 with added PMB/Boc deprotection protocol, using 4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutanoic acid (9.3 mg, 0.017 mmoles) and 4-methoxybenzyl 6-(3-(benzo[d]thiazol-2-ylamino)-4-methyl-6,7-dihydropyrido[2,3-c]pyridazin-8(5H)-yl)-3-(1-(((1 r,3R,5S,7s)-3,5-dimethyl-7-(2-(piperazin-1-yl)ethoxy)adamantan-1-yl)methyl)-5-methyl-1H-pyrazol-4-yl)picolinate (15 mg, 0.016 mmoles), 6-(3-(benzo[d]thiazol-2-ylamino)-4-methyl-6,7-dihydropyrido[2,3-c]pyridazin-8(5H)-yl)-3-(1-(((1 r,3s,5R,7S)-3-(2-(4-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutanoyl)piperazin-1-yl)ethoxy)-5,7-dimethyl adamantan-1-yl)methyl)-5-methyl-1H-pyrazol-4-yl)picolinic acid was obtained as the ammonium salt. HRMS: MH+=1329.6400; Rt=2.09 min (5 min acidic method).

### Example 50: Synthesis of 6-(3-(benzo[d]thiazol-2-ylamino)-4-methyl-6,7-dihydropyrido [2,3-c]pyridazin-8(5H)-yl)-3-(1-(((1r,3s,5R,7S)-3-(2-(4-(2-(2-(((2S,4R)-1-((S)-2-(1-fluoro cyclopropane-1-carboxamido)-3,3-dimethylbutanoyl)-4-hydroxypyrrolidine-2-carboxamido)methyl)-5-(4-methylthiazol-5-yl)phenoxy)acetyl)piperazin-1-yl)ethoxy)-5,7-dimethyl adamantan-1-yl)methyl)-5-methyl-1H-pyrazol-4-yl)picolinic acid

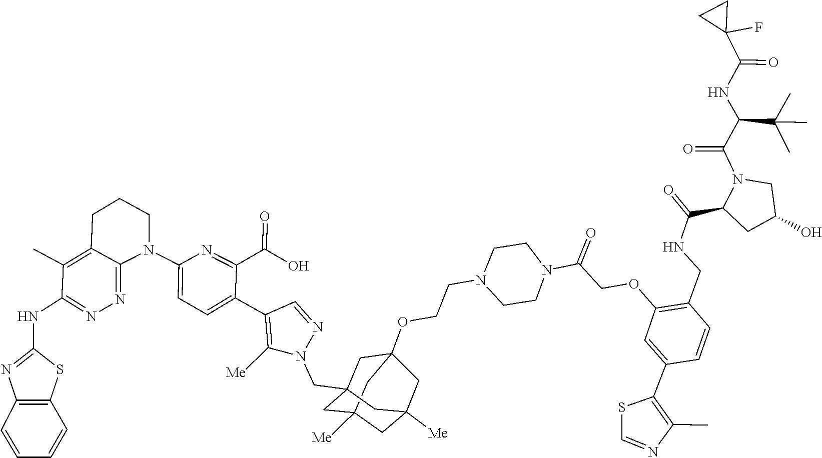 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)N2CCN(CCOC34CC5(C)CC(C)(CC(Cn6ncc(-c7ccc(N8CCCc9c8nnc(Nc8nc%10ccccc%10s8)c9C)nc7C(=O)O)c6C)(C5)C3)C4)CC2)c1]

Following General Procedure 4 with added PMB/Boc deprotection protocol, using 2-(2-(((2S,4R)-1-((S)-2-(1-fluorocyclopropane-1-carboxamido)-3,3-dimethylbutanoyl)-4-hydroxy pyrrolidine-2-carboxamido)methyl)-5-(4-methylthiazol-5-yl)phenoxy)acetic acid (9.6 mg, 0.016 mmoles) and 4-methoxybenzyl 6-(3-(benzo[d]thiazol-2-ylamino)-4-methyl-6,7-dihydropyrido[2,3-c]pyridazin-8(5H)-yl)-3-(1-(((1 r,3R,5S,7s)-3,5-dimethyl-7-(2-(piperazin-1-yl)ethoxy)adamantan-1-yl)methyl)-5-methyl-1H-pyrazol-4-yl)picolinate (15 mg, 0.016 mmoles), 6-(3-(benzo[d]thiazol-2-ylamino)-4-methyl-6,7-dihydropyrido[2,3-c]pyridazin-8(5H)-yl)-3-(1-(((1 r,3s,5R,7S)-3-(2-(4-(2-(2-(((2S,4R)-1-((S)-2-(1-fluorocyclopropane-1-carboxamido)-3,3-dimethylbutanoyl)-4-hydroxy pyrrolidine-2-carboxamido)methyl)-5-(4-methylthiazol-5-yl)phenoxy)acetyl)piperazin-1-yl)ethoxy)-5,7-dimethyladamantan-1-yl)methyl)-5-methyl-1H-pyrazol-4-yl)picolinic acid was obtained as the ammonium salt. HRMS: MH+=1375.6300; Rt=2.14 min (5 min acidic method).

### Example 51: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(12-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-12-oxododecyl)piperazin-1-yl)prop-1-yn-1-yl)phenoxy)propyl)thiazole-4-carboxylic acid

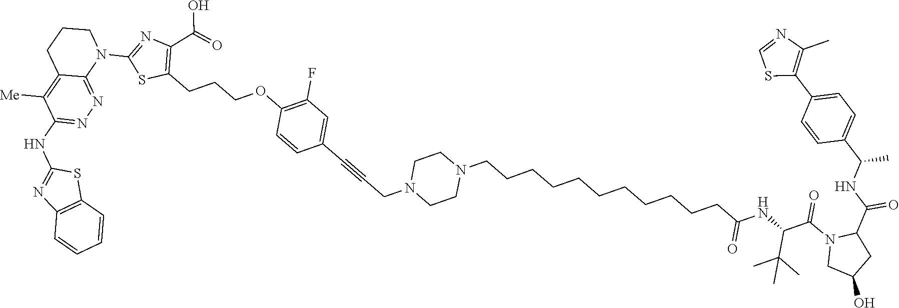 [SMILES: Cc1ncsc1-c1ccc([C@H](C)NC(=O)C2C[C@@H](O)CN2C(=O)[C@@H](NC(=O)CCCCCCCCCCCN2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1]

### General Procedure 5:

To 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl)phenoxy)propyl)thiazole-4-carboxylic acid (18 mg, 0.026 mmoles) and (2S,4R)-1-((S)-3,3-dimethyl-2-(12-oxododecanamido)butanoyl)-4-hydroxy-N—((S)-1-(4-(4-methylthiazol-5-yl)phenyl)ethyl)pyrrolidine-2-carboxamide (18.2 mg, 0.028 mmoles) in DMF (0.66 mL) was added acetic acid (9.3 uL, 0.155 mmoles) followed by sodium triacetoxyborohydride (8.2 mg, 0.039 mmoles). After stirring overnight, the solution was diluted with DMSO (2.3 mL) and purified by RP-HPLC ISCO gold chromatography (10-100% MeCN/H2O, 0.1% NH4OH modifier). Upon lyophilization, 2-(3-(benzo[d]thiazol-2-ylamino)-4-methyl-6,7-dihydropyrido[2,3-c]pyridazin-8(5H)-yl)-5-(3-(2-fluoro-4-(3-(4-(12-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-12-oxododecyl)piperazin-1-yl)prop-1-yn-1-yl)phenoxy)propyl)thiazole-4-carboxylic acid (21.0 mg, 0.016 mmoles) was obtained as the ammonium salt. HRMS: MH+=1324.5900; Rt=2.66 min (5 min acidic method).

### Example 52: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl) amino)-9-oxononyl)piperazin-1-yl)prop-1-yn-1-yl)phenoxy)propyl)thiazole-4-carboxylic acid

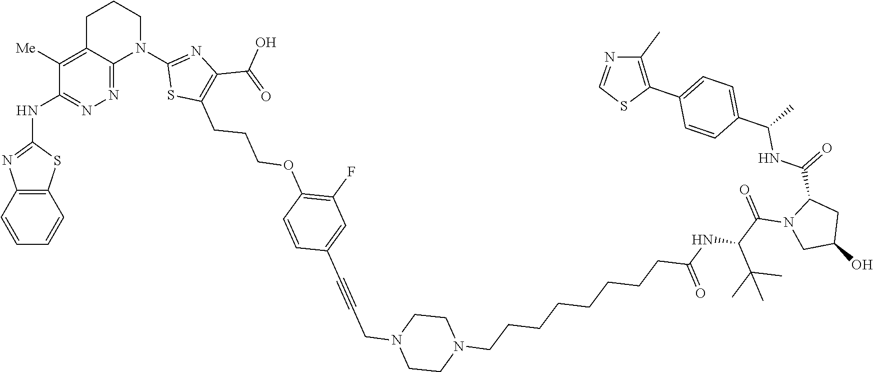 [SMILES: Cc1ncsc1-c1ccc([C@H](C)NC(=O)[C@@H]2C[C@@H](O)CN2C(=O)[C@@H](NC(=O)CCCCCCCCN2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1]

Following General Procedure 5 and using 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl) phenoxy)propyl)thiazole-4-carboxylic acid (16 mg, 0.023 mmoles) and (2S,4R)-1-((S)-3,3-dimethyl-2-(9-oxononanamido)butanoyl)-4-hydroxy-N—((S)-1-(4-(4-methylthiazol-5-yl)phenyl) ethyl)pyrrolidine-2-carboxamide (15.1 mg, 0.025 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononyl)piperazin-1-yl)prop-1-yn-1-yl)phenoxy)propyl)thiazole-4-carboxylic acid was obtained as the ammonium salt. HRMS: MH+=1281.5500; Rt=2.41 min (5 min acidic method).

### Example 53: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(5-(2-(2-(((2S,4R)-1-((S)-2-(1-fluorocyclo propane-1-carboxamido)-3,3-dimethylbutanoyl)-4-hydroxypyrrolidine-2-carboxamido) methyl)-5-(4-methylthiazol-5-yl)phenoxy)acetamido)pentyl)piperazin-1-yl)prop-1-yn-1-yl)phenoxy)propyl)thiazole-4-carboxylic acid

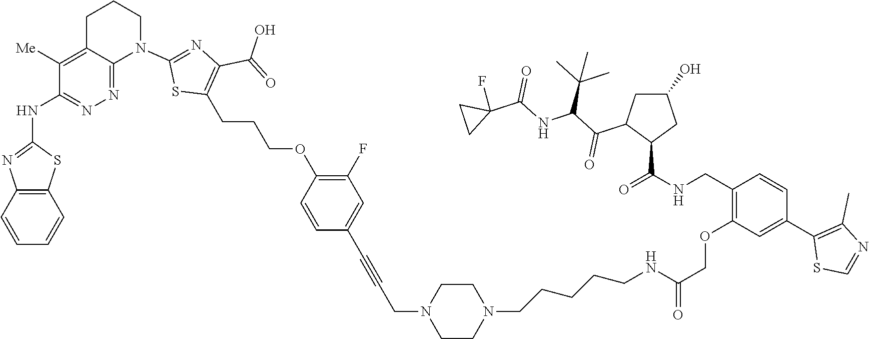

Following General Procedure 5 and using 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl) phenoxy)propyl)thiazole-4-carboxylic acid (14.5 mg, 0.021 mmoles) and (2S,4R)-1-((S)-2-(1-fluorocyclopropane-1-carboxamido)-3,3-dimethylbutanoyl)-4-hydroxy-N-(4-(4-methylthiazol-5-yl)-2-(2-oxo-2-((5-oxopentyl)amino)ethoxy)benzyl)pyrrolidine-2-carboxamide (18.1 mg, 0.023 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(5-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy) acetamido)pentyl)piperazin-1-yl)prop-1-yn-1-yl)phenoxy)propyl)thiazole-4-carboxylic acid was obtained as the ammonium salt. HRMS: MH+=1356.5200; Rt=2.32 min (5 min acidic method).

### Example 54: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(5-(2-(2-(((2S,4R)-1-((S)-2-(1-fluorocyclo propane-1-carboxamido)-3,3-dimethylbutanoyl)-4-hydroxypyrrolidine-2-carboxamido) methyl)-5-(4-methylthiazol-5-yl)phenoxy)-N-methylacetamido)pentyl)piperazin-1-yl)prop-1-yn-1-yl)phenoxy)propyl)thiazole-4-carboxylic acid

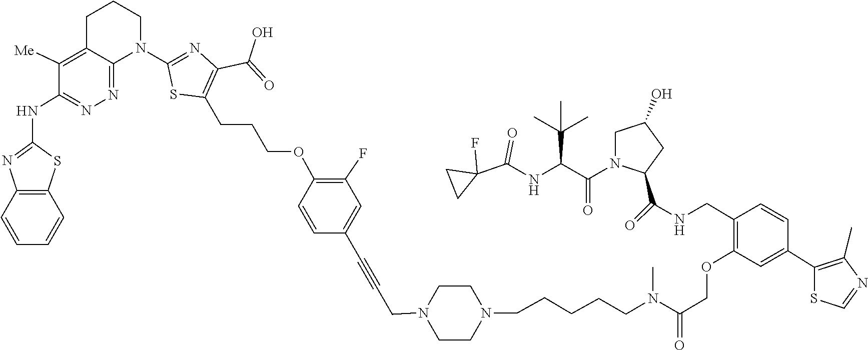 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)N(C)CCCCCN2CCN(CC#Cc3ccc(OCCCc4sc(N5CCCc6c5nnc(Nc5nc7ccccc7s5)c6C)nc4C(=O)O)c(F)c3)CC2)c1]

Following General Procedure 5 and using 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl) phenoxy)propyl)thiazole-4-carboxylic acid (30 mg, 0.043 mmoles) and (2S,4R)-1-((S)-2-(1-fluorocyclopropane-1-carboxamido)-3,3-dimethylbutanoyl)-4-hydroxy-N-(2-(2-(methyl(5-oxopentyl)amino)-2-oxoethoxy)-4-(4-methylthiazol-5-yl)benzyl)pyrrolidine-2-carboxamide (32.5 mg, 0.047 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(5-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-N-methylacetamido)pentyl)piperazin-1-yl)prop-1-yn-1-yl) phenoxy)propyl)thiazole-4-carboxylic acid was obtained as the ammonium salt. HRMS: MH+=1370.5601; Rt=2.22 min (5 min acidic method).

### Example 55: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(12-(2-(((2S,4R)-1-((S)-2-(1-fluorocyclo propane-1-carboxamido)-3,3-dimethylbutanoyl)-4-hydroxypyrrolidine-2-carboxamido) methyl)-5-(4-methylthiazol-5-yl)phenoxy)dodecyl)piperazin-1-yl)prop-1-yn-1-yl)phenoxy) propyl)thiazole-4-carboxylic acid

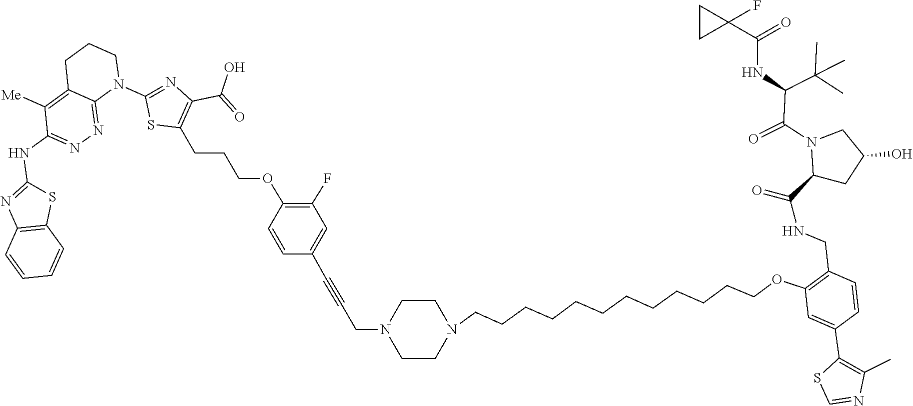 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCCCN2CCN(CC#Cc3ccc(OCCCc4sc(N5CCCc6c5nnc(Nc5nc7ccccc7s5)c6C)nc4C(=O)O)c(F)c3)CC2)c1]

Following General Procedure 5 and using 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl) phenoxy)propyl)thiazole-4-carboxylic acid (20 mg, 0.029 mmoles) and (2S,4R)-1-((S)-2-(1-fluorocyclopropane-1-carboxamido)-3,3-dimethylbutanoyl)-4-hydroxy-N-(4-(4-methylthiazol-5-yl)-2-((12-oxododecyl)oxy)benzyl)pyrrolidine-2-carboxamide (25 mg, 0.031 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(12-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)dodecyl)piperazin-1-yl)prop-1-yn-1-yl)phenoxy)propyl)thiazole-4-carboxylic acid was obtained as the ammonium salt. HRMS: MH+=1397.6200; Rt=2.60 min (5 min acidic method).

### Example 56: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(8-(2-(((2S,4R)-1-((S)-2-(1-fluorocyclo propane-1-carboxamido)-3,3-dimethylbutanoyl)-4-hydroxypyrrolidine-2-carboxamido) methyl)-5-(4-methylthiazol-5-yl)phenoxy)octyl)piperazin-1-yl)prop-1-yn-1-yl)phenoxy) propyl)thiazole-4-carboxylic acid

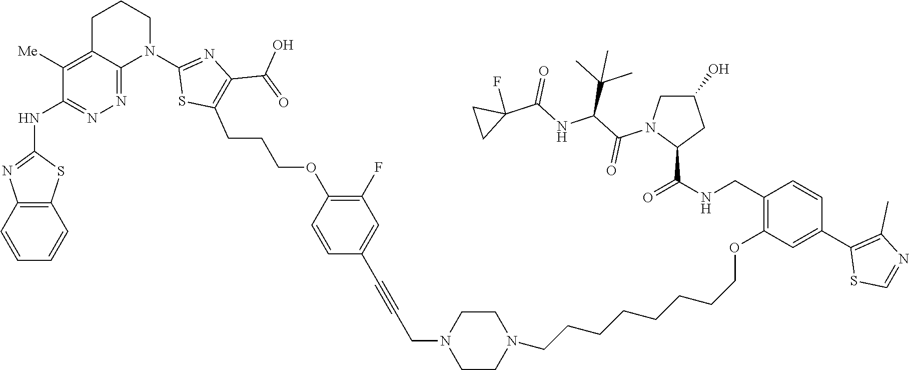 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCN2CCN(CC#Cc3ccc(OCCCc4sc(N5CCCc6c5nnc(Nc5nc7ccccc7s5)c6C)nc4C(=O)O)c(F)c3)CC2)c1]

Following General Procedure 5 and using 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl) phenoxy)propyl)thiazole-4-carboxylic acid (13.0 mg, 0.019 mmoles) and (2S,4R)-1-((S)-2-(1-fluorocyclopropane-1-carboxamido)-3,3-dimethylbutanoyl)-4-hydroxy-N-(4-(4-methylthiazol-5-yl)-2-((8-oxooctyl)oxy)benzyl)pyrrolidine-2-carboxamide (13.2 mg, 0.020 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(8-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)octyl)piperazin-1-yl)prop-1-yn-1-yl)phenoxy)propyl)thiazole-4-carboxylic acid was obtained as the ammonium salt. HRMS: MH+=1341.5500; Rt=2.38 min (5 min acidic method).

### Example 57: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(9-(2-(((2S,4R)-1-((S)-2-(1-fluorocyclo propane-1-carboxamido)-3,3-dimethylbutanoyl)-4-hydroxypyrrolidine-2-carboxamido) methyl)-5-(4-methylthiazol-5-yl)phenoxy)nonyl)piperazin-1-yl)prop-1-yn-1-yl)phenoxy) propyl)thiazole-4-carboxylic acid

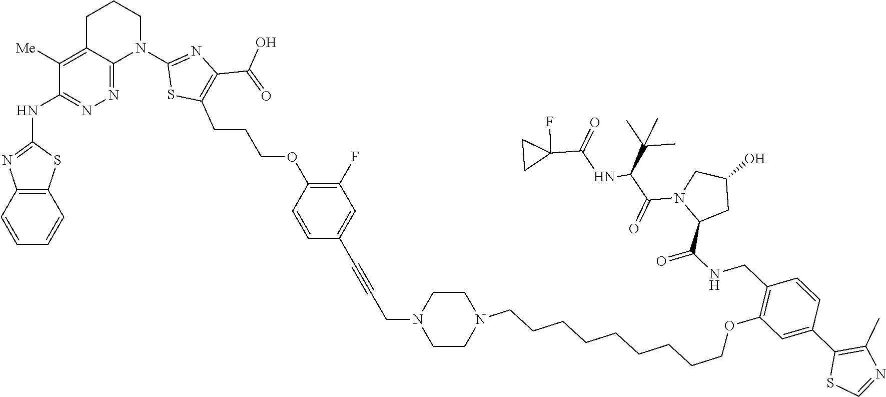 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCN2CCN(CC#Cc3ccc(OCCCc4sc(N5CCCc6c5nnc(Nc5nc7ccccc7s5)c6C)nc4C(=O)O)c(F)c3)CC2)c1]

Following General Procedure 5 and using 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl) phenoxy)propyl)thiazole-4-carboxylic acid (20 mg, 0.029 mmoles) and (2S,4R)-1-((S)-2-(1-fluorocyclopropane-1-carboxamido)-3,3-dimethylbutanoyl)-4-hydroxy-N-(4-(4-methylthiazol-5-yl)-2-((9-oxononyl)oxy)benzyl)pyrrolidine-2-carboxamide (21.8 mg, 0.031 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(9-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)nonyl)piperazin-1-yl)prop-1-yn-1-yl)phenoxy)propyl)thiazole-4-carboxylic acid was obtained as the ammonium salt. HRMS: MH+=1355.5800; Rt=2.43 min (5 min acidic method).

### Example 58: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(4-((2-(((2S,4R)-1-((S)-2-(1-fluorocyclo propane-1-carboxamido)-3,3-dimethylbutanoyl)-4-hydroxypyrrolidine-2-carboxamido) methyl)-5-(4-methylthiazol-5-yl)phenoxy)methyl)benzyl)piperazin-1-yl)prop-1-yn-1-yl)phenoxy)propyl)thiazole-4-carboxylic acid

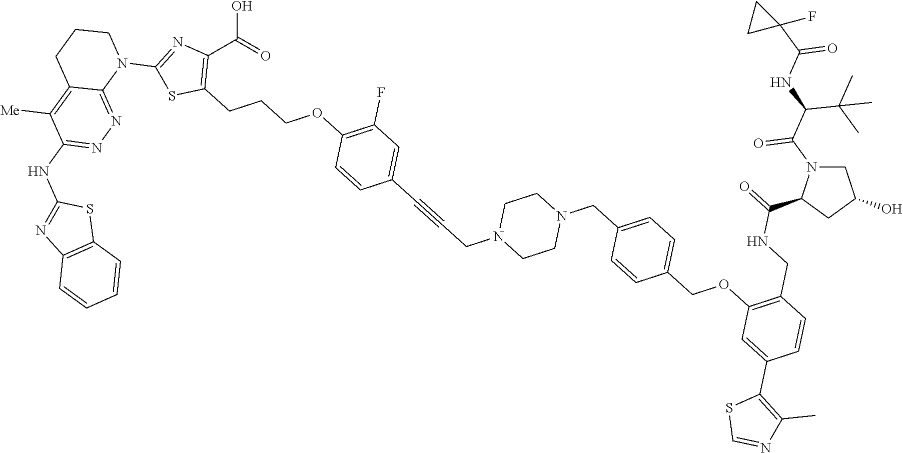 [SMILES: Cc1ncsc1-c1ccc(CNC(=O)[C@@H]2C[C@@H](O)CN2C(=O)[C@@H](NC(=O)C2(F)CC2)C(C)(C)C)c(OCc2ccc(CN3CCN(CC#Cc4ccc(OCCCc5sc(N6CCCc7c6nnc(Nc6nc8ccccc8s6)c7C)nc5C(=O)O)c(F)c4)CC3)cc2)c1]

Following General Procedure 5 and using 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl) phenoxy)propyl)thiazole-4-carboxylic acid (17 mg, 0.024 mmoles) and (2S,4R)-1-((S)-2-(1-fluorocyclopropane-1-carboxamido)-3,3-dimethylbutanoyl)-N-(2-((4-formylbenzyl)oxy)-4-(4-methylthiazol-5-yl)benzyl)-4-hydroxypyrrolidine-2-carboxamide (17.4 mg, 0.027 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(4-((2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)methyl) benzyl) piperazin-1-yl)prop-1-yn-1-yl)phenoxy)propyl)thiazole-4-carboxylic acid was obtained as the ammonium salt. HRMS: MH+=1334.5000; Rt=2.28 min (5 min acidic method).

### Synthesis of 2-(3-(benzo[d]thiazol-2-ylamino)-4-methyl-6,7-dihydropyrido[2,3-c]pyridazin-8(5H)-yl)-5-(3-(2-fluoro-4-(3-(4-(methylglycyl)piperazin-1-yl)prop-1-yn-1-yl)phenoxy) propyl)thiazole-4-carboxylic acid

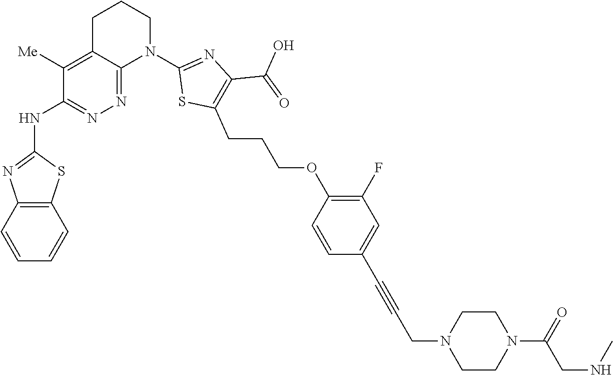 [SMILES: CNCC(=O)N1CCN(CC#Cc2ccc(OCCCc3sc(N4CCCc5c4nnc(Nc4nc6ccccc6s4)c5C)nc3C(=O)O)c(F)c2)CC1]

Following General Procedure 4 with added PMB/Boc deprotection protocol, using 2-(3-(benzo[d]thiazol-2-ylamino)-4-methyl-6,7-dihydropyrido[2,3-c]pyridazin-8(5H)-yl)-5-(3-(2-fluoro-4-(3-(piperazin-1-yl)prop-1-yn-1-yl)phenoxy)propyl)thiazole-4-carboxylic acid (50 mg, 0.072 mmoles) and N-(tert-butoxycarbonyl)-N-methylglycine (13.54 mg, 0.072 mmoles), 2-(3-(benzo[d]thiazol-2-ylamino)-4-methyl-6,7-dihydropyrido[2,3-c]pyridazin-8(5H)-yl)-5-(3-(2-fluoro-4-(3-(4-(methylglycyl)piperazin-1-yl)prop-1-yn-1-yl)phenoxy)propyl)thiazole-4-carboxylic acid was obtained as the ammonium salt. LC/MS (M+2H+)/2=770.6; Rt=1.54 min (5 min acidic method).

### Example 59: Synthesis of 2-(3-(benzo[d]thiazol-2-ylamino)-4-methyl-6,7-dihydropyrido [2,3-c]pyridazin-8(5H)-yl)-5-(3-(2-fluoro-4-(3-(4-(N-(2-(2-(((2S,4R)-1-((S)-2-(1-fluorocyclo propane-1-carboxamido)-3,3-dimethylbutanoyl)-4-hydroxypyrrolidine-2-carboxamido) methyl)-5-(4-methylthiazol-5-yl)phenoxy)ethyl)-N-methylglycyl)piperazin-1-yl)prop-1-yn-1-yl)phenoxy)propyl)thiazole-4-carboxylic acid

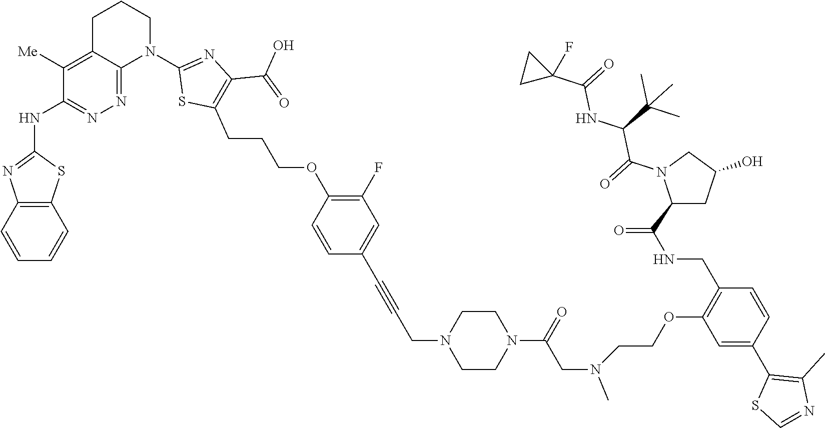 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN(C)CC(=O)N2CCN(CC#Cc3ccc(OCCCc4sc(N5CCCc6c5nnc(Nc5nc7ccccc7s5)c6C)nc4C(=O)O)c(F)c3)CC2)c1]

Following General Procedure 5, using 2-(3-(benzo[d]thiazol-2-ylamino)-4-methyl-6,7-dihydropyrido[2,3-c]pyridazin-8(5H)-yl)-5-(3-(2-fluoro-4-(3-(4-(methylglycyl)piperazin-1-yl)prop-1-yn-1-yl)phenoxy)propyl)thiazole-4-carboxylic acid (8.0 mg, 0.01 mmoles) and (2S,4R)-1-((S)-2-(1-fluorocyclopropane-1-carboxamido)-3,3-dimethylbutanoyl)-4-hydroxy-N-(4-(4-methylthiazol-5-yl)-2-(2-oxoethoxy)benzyl)pyrrolidine-2-carboxamide (6.0 mg, 0.01 mmoles), 2-(3-(benzo[d] thiazol-2-ylamino)-4-methyl-6,7-dihydropyrido[2,3-c]pyridazin-8(5H)-yl)-5-(3-(2-fluoro-4-(3-(4-(N-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)-N-methyl glycyl)piperazin-1-yl)prop-1-yn-1-yl)phenoxy)propyl)thiazole-4-carboxylic acid was obtained as the ammonium salt. HRMS: MH+=1328.4900; Rt=2.24 min (5 min acidic method).

### Example 60: 6-[3-(1,3-benzothiazol-2-ylamino)-4-methyl-6,7-dihydro-5H-pyrido[2,3-c]pyridazin-8-yl]-3-[1-[[3-[2-[10-[2-(2,6-dioxo-3-piperidyl)-1,3-dioxo-isoindolin-5-yl]oxydecyl-methyl-amino]ethoxy]-5,7-dimethyl-1-adamantyl]methyl]-5-methyl-pyrazol-4-yl]pyridine-2-carboxylic acid

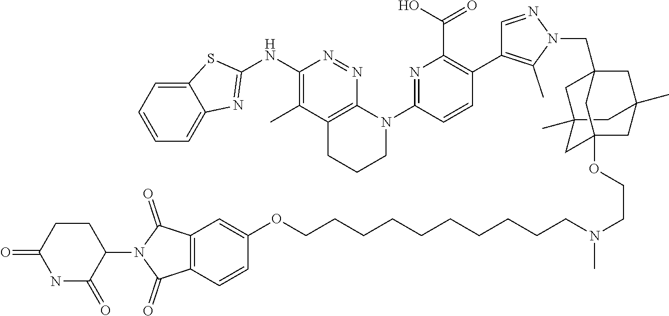 [SMILES: Cc1c(Nc2nc3ccccc3s2)nnc2c1CCCN2c1ccc(-c2cnn(CC34CC5(C)CC(C)(C3)CC(OCCN(C)CCCCCCCCCCOc3ccc6c(c3)C(=O)N(C3CCC(=O)NC3=O)C6=O)(C5)C4)c2C)c(C(=O)O)n1]

### Step A: 5-(10-bromodecoxy)-2-(2,6-dioxo-3-piperidyl)isoindoline-1,3-dione

Using General procedure for the alkylation of the hydroxy-thalidomide starting from 2-(2,6-dioxo-3-piperidyl)-5-hydroxy-isoindoline-1,3-dione (0.44 mmol) and 1,10-dibromodecane as the appropriate alkylating agent, 163 mg of the desired product was obtained. ¹H NMR (500 MHz, DMSO-d₆) δ ppm 11.12 (s, 1H), 7.83 (d, 1H), 7.42 (d, 1H), 7.34 (dd, 1H), 5.12 (dd, 1H), 4.16 (t, 2H), 3.52 (t, 2H), 2.89/2.59 (td+dd, 2H), 2.52/2.04 (dd+dt, 2H), 1.78 (qn, 2H), 1.75 (qn, 2H), 1.42 (qn, 2H), 1.37 (qn, 2H), 1.35-1.22 (in, 8H); 130 NMR (125 MHz, DMSO-d₆) δ ppm 173.3, 170.4, 167.4, 167.3, 164.6, 134.4, 125.8, 123.3, 121.1, 109.3, 69.3, 49.4, 35.7, 32.7, 31.4, 28.8, 28.0, 25.8, 22.5; HRMS-ESI (m/z): [M+H]⁺ calcd for C₂₃H₃₀BrN₂O₈s: 493.1338. found: 493.1333.

### Step B: 6-[3-(1,3-benzothiazol-2-ylamino)-4-methyl-6,7-dihydro-5H-pyrido[2,3-c]pyridazin-8-yl]-3-[1-[[3-[2-[10-[2-(2,6-dioxo-3-piperidyl)-1,3-dioxo-isoindolin-5-yl]oxydecyl-methyl-amino]ethoxy]-5,7-dimethyl-1-adamantyl]methyl]-5-methyl-pyrazol-4-yl]pyridine-2-carboxylic acid

Using the General procedure for the production of thalidomide-based degraders via alkylation starting from the product of Preparation 4 (35 mg) and the product of Step A as the appropriate alkylating agent, 9 mg of the desired product was obtained. HRMS-ESI (m/z): [M+H]⁺ calcd for C₆₄H₇₈N₁₁O₈S: 1160.5756. found: 1160.5749.

### Example 61: 6-[[6-(1,3-benzothiazol-2-ylamino)-5-methyl-pyridazin-3-yl]-[5-[4-[2-[2-[[2-(2,6-dioxo-3-piperidyl)-1,3-dioxo-isoindolin-4-yl]amino]ethoxy]ethoxymethyl]triazol-1-yl]pentyl]amino]-3-[1-[[3-[2-(dimethylamino)ethoxy]-5,7-dimethyl-1-adamantyl]methyl]-5-methyl-pyrazol-4-yl]pyridine-2-carboxylic acid

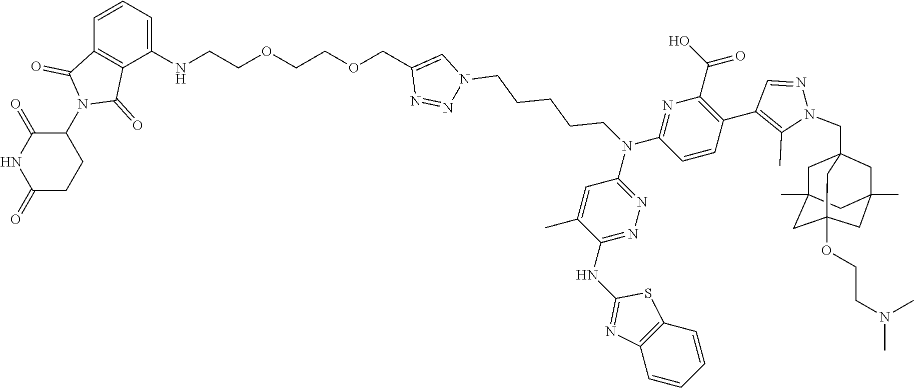 [SMILES: Cc1cc(N(CCCCCn2cc(COCCOCCNc3cccc4c3C(=O)N(C3CCC(=O)NC3=O)C4=O)nn2)c2ccc(-c3cnn(CC45CC6(C)CC(C)(C4)CC(OCCN(C)C)(C6)C5)c3C)c(C(=O)O)n2)nnc1Nc1nc2ccccc2s1]

To a solution of CuSO₄ 5H₂O (3.20 mg, 0.018 mmol) in water (0.6 mL) was added THTPA (7.82 mg, 1 eq). This solution was added to a suspension of Preparation 16 (15.0 mg, 1 eq), 2-(2,6-dioxo-3-piperidyl)-4-[2-(2-prop-2-ynoxyethoxy)ethylamino]isoindoline-1,3-dione (described in WO2020081880A, 12.94 mg, 1.8 eq), and Na-L-ascorbate (3.57 mg, 1 eq) in DMSO (1.5 mL). After stirring for 3 h, the reaction was filtered and purified by preparative HPLC (CHS column, TFA method) to give the desired product (1.7 mg, 7%). HRMS (ESI) [M+H]⁺ found=1232.5765.

### Example 62: 6-[[6-(1,3-benzothiazol-2-ylamino)-5-methyl-pyridazin-3-yl]-[5-[4-[2-[2-[2-[[2-(2,6-dioxo-3-piperidyl)-1,3-dioxo-isoindolin-4-yl]amino]ethoxy]ethoxy]ethoxymethyl] triazol-1-yl]pentyl]amino]-3-[1-[[3-[2-(dimethylamino)ethoxy]-5,7-dimethyl-1-adamantyl] methyl]-5-methyl-pyrazol-4-yl]pyridine-2-carboxylic acid

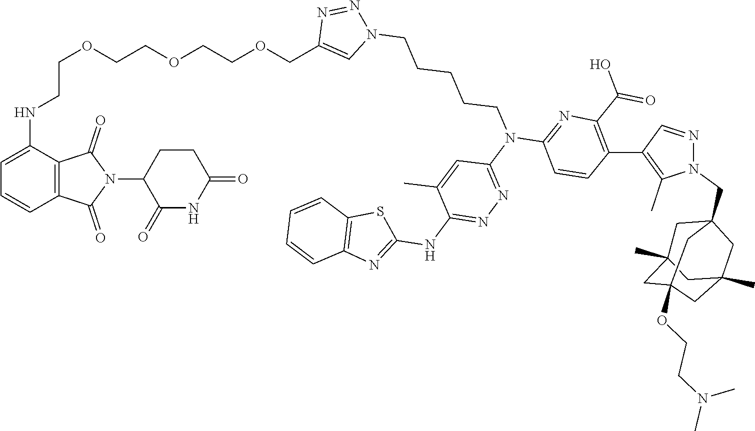 [SMILES: Cc1cc(N(CCCCCn2cc(COCCOCCOCCNc3cccc4c3C(=O)N(C3CCC(=O)NC3=O)C4=O)nn2)c2ccc(-c3cnn(C[C@@]45C[C@]6(C)C[C@](C)(C4)C[C@](OCCN(C)C)(C6)C5)c3C)c(C(=O)O)n2)nnc1Nc1nc2ccccc2s1]

The product was synthesized according to the procedure described for Example 61, using 2-(2,6-dioxo-3-piperidyl)-4-[2-[2-(2-prop-2-ynoxyethoxy)ethoxy]ethylamino]isoindoline-1,3-dione as an appropriate acetylene (84%). HRMS (ESI) [M+H]⁺ found=1276.6040.

### Example 63: 6-[[6-(1,3-benzothiazol-2-ylamino)-5-methyl-pyridazin-3-yl]-[5-[4-[2-[2-[2-[[2-[2-(2,6-dioxo-3-piperidyl)-1,3-dioxo-isoindolin-4-yl]oxyacetyl]amino]ethoxy]ethoxy]ethoxy methyl]triazol-1-yl]pentyl]amino]-3-[1-[[3-[2-(dimethylamino)ethoxy]-5,7-dimethyl-1-adamantyl]methyl]-5-methyl-pyrazol-4-yl]pyridine-2-carboxylic acid

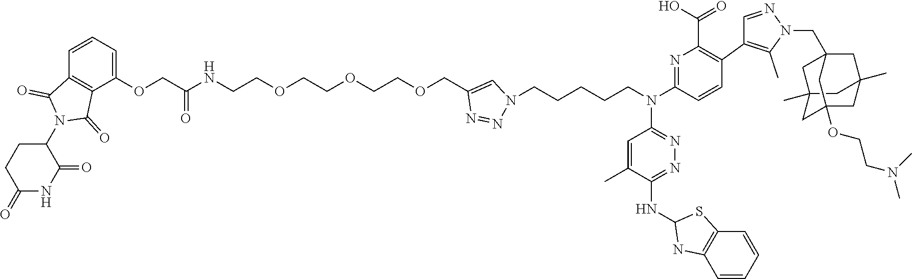 [SMILES: Cc1cc(N(CCCCCn2cc(COCCOCCOCCNC(=O)COc3cccc4c3C(=O)N(C3CCC(=O)NC3=O)C4=O)nn2)c2ccc(-c3cnn(CC45CC6(C)CC(C)(C4)CC(OCCN(C)C)(C6)C5)c3C)c(C(=O)O)n2)nnc1NC1Nc2ccccc2S1]

### Step A: 2-[2-(2,6-dioxo-3-piperidyl)-1,3-dioxo-isoindolin-4-yl]oxy-N-[2-[2-(2-prop-2-ynoxyethoxy)ethoxy]ethyl]acetamide

A solution of 2-[2-(2,6-dioxo-3-piperidyl)-1,3-dioxo-isoindolin-4-yl]oxyacetic acid (200 mg, 0.60 mmol), 2-[2-(2-prop-2-ynoxyethoxy)ethoxy]ethanamine (0.13 mL, 0.60 mmol), HATU (228.8 mg, 0.60 mmol), HOAt (81.9 mg, 0.60 mmol), and DIEA (0.54 mL, 3.00 mmol) in DMSO (6 mL) was stirred for 2 h. The reaction was purified by preparative HPLC (Xbridge Column, TFA method) to give 218 mg (72%) of the desired product. ¹H NMR (400 MHz, dmso-d6) δ ppm 11.11 (s, 1H), 7.99 (t, 1H), 7.81 (dd, 1H), 7.50/7.40 (2d, 2H), 5.11 (dd, 1H), 4.79 (s, 2H), 4.12 (d, 2H), 3.52 (m, 8H), 3.47 (t, 2H), 3.40 (t, 1H), 3.32 (quad, 2H), 2.90/2.57 (ddd+m, 2H), 2.57/2.04 (2m, 2H); IR: 3700-2700, 1776/1703/1653, 746.

### Step B: (4-methoxyphenyl)methyl 3-[1-[[3-[2-(dimethylamino)ethoxy]-5,7-dimethyl-1-adamantyl]methyl]-5-methyl-pyrazol-4-yl]-6-[5-[4-[2-[2-[2-[[2-[2-(2,6-dioxo-3-piperidyl)-1,3-dioxo-isoindolin-4-yl]oxyacetyl]amino]ethoxy]ethoxy]ethoxymethyl]triazol-1-yl]pentyl-[5-methyl-6-[(Z)-[3-(2-trimethylsilylethoxymethyl)-1,3-benzothiazol-2-ylidene]amino] pyridazin-3-yl]amino]pyridine-2-carboxylate

The product was synthesized according to the procedure described for Step D of Example 28, using Step A as an appropriate acetylene (52%).

### Step C: 6-[[6-(1,3-benzothiazol-2-ylamino)-5-methyl-pyridazin-3-yl]-[5-[4-[2-[2-[2-[[2-[2-(2,6-dioxo-3-piperidyl)-1,3-dioxo-isoindolin-4-yl]oxyacetyl]amino]ethoxy]ethoxy]ethoxy methyl]triazol-1-yl]pentyl]amino]-3-[1-[[3-[2-(dimethylamino)ethoxy]-5,7-dimethyl-1-adamantyl]methyl]-5-methyl-pyrazol-4-yl]pyridine-2-carboxylic acid

The product was synthesized according to the procedure described for Step E of Example 28, using Step B as a starting material (56%). HRMS (ESI) [M+H]⁺ found=1334.6067.

### Example 64: 6-[[6-(1,3-benzothiazol-2-ylamino)-5-methyl-pyridazin-3-yl]-[5-[4-[7-[[2-[2-(2,6-dioxo-3-piperidyl)-1,3-dioxo-isoindolin-4-yl]oxyacetyl]amino]heptyl]triazol-1-yl]pentyl]amino]-3-[1-[[3-[2-(dimethylamino)ethoxy]-5,7-dimethyl-1-adamantyl]methyl]-5-methyl-pyrazol-4-yl]pyridine-2-carboxylic acid

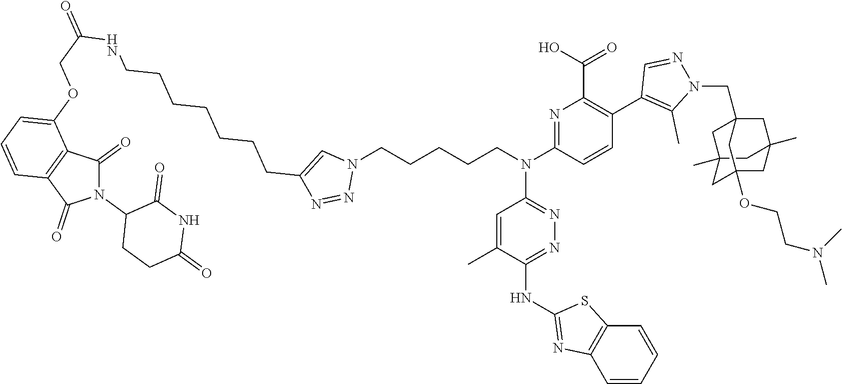

### Step A: 2-[2-(2,6-dioxo-3-piperidyl)-1,3-dioxo-isoindolin-4-yl]oxy-N-non-8-ynyl-acetamide

The product was synthesized according to the procedure described for Step A of Example 27, using non-8-yn-1-amine as an appropriate amine (82%). ¹H NMR (400 MHz, dmso-d6) δ ppm 11.10 (s, 1H), 7.93 (m, 1H), 7.81 (dd, 1H), 7.50 (d, 1H), 7.40 (d, 1H), 5.10 (s, 1H), 4.76 (s, 2H), 3.14 (m, 2H), 2.89 (m, 1H), 2.71 (s, 1H), 2.60 (m, 1H), 2.53 (m, 1H), 2.13 (m, 2H), 2.03 (m, 1H), 1.35 (m, 10H); IR: 3390-3100, 1776/1728/1708/1660, 747.

### Step B: (4-methoxyphenyl)methyl 3-[1-[[3-[2-(dimethylamino)ethoxy]-5,7-dimethyl-1-adamantyl]methyl]-5-methyl-pyrazol-4-yl]-6-[5-[4-[7-[[2-[2-(2,6-dioxo-3-piperidyl)-1,3-dioxo-isoindolin-4-yl]oxyacetyl]amino]heptyl]triazol-1-yl]pentyl-[5-methyl-6-[(Z)-[3-(2-trimethylsilylethoxymethyl)-1,3-benzothiazol-2-ylidene]amino]pyridazin-3-yl]amino]pyridine-2-carboxylate

The product was synthesized according to the procedure described for Step D of Example 28, using Step A as an appropriate acetylene (62%).

### Step C: 6-[[6-(1,3-benzothiazol-2-ylamino)-5-methyl-pyridazin-3-yl]-[5-[4-[7-[[2-[2-(2,6-dioxo-3-piperidyl)-1,3-dioxo-isoindolin-4-yl]oxyacetyl]amino]heptyl]triazol-1-yl]pentyl]amino]-3-[1-[[3-[2-(dimethylamino)ethoxy]-5,7-dimethyl-1-adamantyl]methyl]-5-methyl-pyrazol-4-yl]pyridine-2-carboxylic acid

The product was synthesized according to the procedure described for Step E of Example 28, using Step B as a starting material (51%). HRMS (ESI) [M+H]⁺ found=1286.6223.

### Example 65: 6-[[6-(1,3-benzothiazol-2-ylamino)-5-methyl-pyridazin-3-yl]-[5-[4-[2-[2-[[2-[2-(2,6-dioxo-3-piperidyl)-1,3-dioxo-isoindolin-4-yl]oxyacetyl]amino]ethoxy]ethoxy methyl]triazol-1-yl]pentyl]amino]-3-[1-[[3-[2-(dimethylamino)ethoxy]-5,7-dimethyl-1-adamantyl]methyl]-5-methyl-pyrazol-4-yl]pyridine-2-carboxylic acid

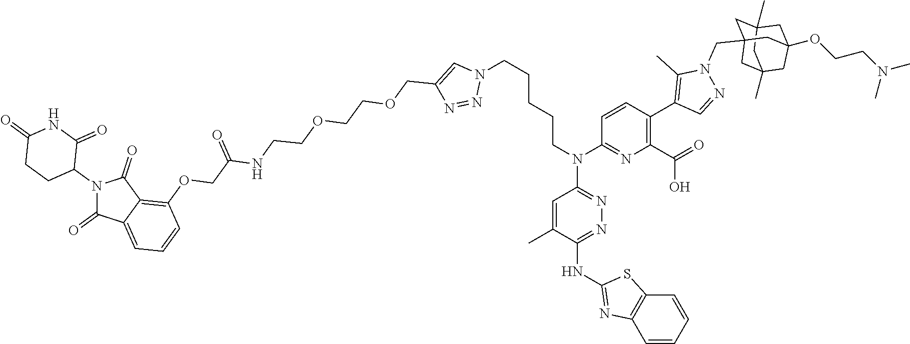

### Step A: 2-[2-(2,6-dioxo-3-piperidyl)-1,3-dioxo-isoindolin-4-yl]oxy-N-[2-(2-prop-2-ynoxyethoxy)ethyl]acetamide

The product was synthesized according to the procedure described for Step A of Example 27, using 2-(2-prop-2-ynoxyethoxy)ethanamine as an appropriate amine (84%). ¹H NMR (400 MHz, dmso-d6) δ ppm 11.05/8.05 (m+t, 2H), 7.81 (t, 1H), 7.50 (d, 1H), 7.40 (d, 1H), 5.10 (dd, 1H), 4.79 (s, 2H), 4.12 (s, 2H), 3.55 (m, 4H), 3.45 (t, 2H), 3.40 (tf, 1H), 3.3 (quad, 2H), 2.90/2.60 (20m, 2H), 2.55/2.02 (2m, 2H); IR: 3387+3094, 3245, 2127, 1774+1706+1656, 1619, 1547.

### Step B: (4-methoxyphenyl)methyl 3-[1-[[3-[2-(dimethylamino)ethoxy]-5,7-dimethyl-1-adamantyl]methyl]-5-methyl-pyrazol-4-yl]-6-[5-[4-[2-[2-[[2-[2-(2,6-dioxo-3-piperidyl)-1,3-dioxo-isoindolin-4-yl]oxyacetyl]amino]ethoxy]ethoxymethyl]triazol-1-yl]pentyl-[5-methyl-6-[(Z)-[3-(2-trimethylsilylethoxymethyl)-1,3-benzothiazol-2-ylidene]amino]pyridazin-3-yl]amino]pyridine-2-carboxylate

The product was synthesized according to the procedure described for Step D of Example 28, using Step A as an appropriate acetylene (53%).

### Step C: 6-[[6-(1,3-benzothiazol-2-ylamino)-5-methyl-pyridazin-3-yl]-[5-[4-[2-[2-[[2-[2-(2,6-dioxo-3-piperidyl)-1,3-dioxo-isoindolin-4-yl]oxyacetyl]amino]ethoxy]ethoxymethyl]triazol-1-yl]pentyl]amino]-3-[1-[[3-[2-(dimethylamino)ethoxy]-5,7-dimethyl-1-adamantyl]methyl]-5-methyl-pyrazol-4-yl]pyridine-2-carboxylic acid

The product was synthesized according to the procedure described for Step E of Example 28, using Step B as a starting material (56%). HRMS (ESI) [M+H]⁺ found=1290.5815.

### Example 66: 2-[3-(1,3-benzothiazol-2-ylamino)-4-methyl-6,7-dihydro-5H-pyrido[2,3-c]pyridazin-8-yl]-5-[3-[[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-5-oxo-pentyl]-methyl-amino]propyl]thiazole-4-carboxylic acid

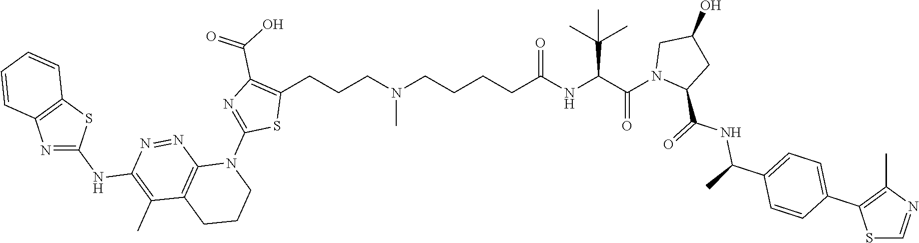 [SMILES: Cc1ncsc1-c1ccc([C@@H](C)NC(=O)[C@@H]2C[C@H](O)CN2C(=O)[C@@H](NC(=O)CCCCN(C)CCCc2sc(N3CCCc4c3nnc(Nc3nc5ccccc5s3)c4C)nc2C(=O)O)C(C)(C)C)cc1]

### Step A: (2S,4R)-1-[(2S)-2-(5-bromopentanoylamino)-3,3-dimethyl-butanoyl]-4-hydroxy-N-[(15)-1-[4-(4-methylthiazol-5-yl)phenyl]ethyl]pyrrolidine-2-carboxamide

Using General procedure for the acylation of VHL ligands starting from (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.42 mmol) and 5-bromopentanoic acid as the appropriate acid, 138 mg of the desired product was obtained. ¹H NMR (500 MHz, DMSO-d₆) δ ppm 8.99 (s, 1H), 8.39 (d, 1H), 7.86 (d, 1H), 7.43 (d, 2H), 7.38 (d, 2H), 5.22 (brs, 1H), 4.91 (qn, 1H), 4.51 (d, 1H), 4.42 (t, 1H), 4.27 (m, 1H), 3.61/3.58 (dd+dd, 2H), 3.53 (t, 2H), 2.45 (s, 3H), 2.29/2.17 (m+m, 2H), 2.01/1.78 (m+m, 2H), 1.77 (qn, 2H), 1.60 (qn, 2H), 1.37 (d, 3H), 0.93 (s, 9H); 130 NMR (125 MHz, DMSO-d₆) δ ppm 152.0, 129.3, 126.9, 69.3, 59.0, 56.9, 56.8, 48.2, 38.2, 35.3, 34.2, 32.2, 26.9, 24.5, 22.9, 16.5; HRMS (ESI) [M+H]⁺ calcd for C₂₈H₄₀BrN₄O₄S: 607.1954. found 607.1950.

### Step B: methyl 5-[3-[[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-5-oxo-pentyl]-methyl-amino]propyl]-2-[4-methyl-3-[(Z)-[3-(2-trimethylsilylethoxymethyl)-1,3-benzothiazol-2-ylidene]amino]-6,7-dihydro-5H-pyrido[2,3-c]pyridazin-8-yl]thiazole-4-carboxylate

The mixture of 75 mg of Preparation 15 (0.11 mmol), the product of Step A (85 mg, 1.2 eq), DIPEA (0.11 mL) in MeCN (1.3 mL) and NMP (0.58 mL) was stirred at 60° C. for 18 h. The product was purified via flash chromatography using DCM, EtOAc and MeOH as eluents to give 40 mg of the desired product (30%). ¹H NMR (500 MHz, DMSO-d₆) δ ppm 8.99 (s, 1H), 8.37 (d, 1H), 7.89 (d, 1H), 7.81 (d, 1H), 7.43 (m, 1H), 7.43 (m, 1H), 7.43 (d, 2H), 7.37 (d, 2H), 7.23 (td, 1H), 5.84 (s, 2H), 5.09 (br., 1H), 4.91 (qn, 1H), 4.51 (d, 1H), 4.40 (t, 1H), 4.26 (br., 2H), 4.26 (br., 1H), 3.83 (s, 3H), 3.72 (t, 2H), 3.60/3.56 (dd+dd, 2H), 3.24-2.97 (m, 2H), 3.24-2.97 (m, 2H), 3.18 (t, 2H), 2.87 (t, 2H), 2.76 (s, 3H), 2.45 (s, 3H), 2.35 (s, 3H), 2.30/2.18 (m+m, 2H), 2.04 (m, 2H), 2.04 (br., 2H), 2.01/1.80 (m+m, 2H), 1.60 (m, 2H), 1.53 (m, 2H), 1.36 (d, 3H), 0.91 (s, 9H), 0.91 (t, 2H), −0.11 (s, 9H); ¹³C NMR (125 MHz, DMSO-d₆) δ ppm 172.0, 152.0, 129.3, 127.1, 126.9, 123.3, 123.1, 111.8, 73.0, 69.2, 66.7, 59.0, 56.9, 56.7, 55.3, 55.1, 52.1, 48.1, 46.4, 39.7, 38.2, 34.6, 26.9, 26.0, 23.8, 23.6, 23.4, 22.9, 22.8, 20.4, 17.8, 16.5, 12.9, 0.9; HRMS (ESI) [M+H]⁺ calcd for C₅₈H₈₀N₁₁O₇S₃Si: 1166.5174. found 1166.5165.

### Step C: 2-[3-(1,3-benzothiazol-2-ylamino)-4-methyl-6,7-dihydro-5H-pyrido[2,3-c]pyridazin-8-yl]-5-[3-[[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-5-oxo-pentyl]-methyl-amino]propyl]thiazole-4-carboxylic acid

After stirring the product of Step B (40 mg, 0.034 mmol) with LiOH×H₂O (14 mg, 10 eq) in THF (0.17 ml) and water (0.17 ml) at 50° C. for 5 h, concentrated HCl (0.7 mL, 250 eq) was added and the mixture was stirred at 50° C. for 30 min. The product was purified via preparative reversed phase chromatography to give the desired product (22 mg, 42%). HRMS (ESI) [M+H]⁺ calcd for C₅₁H₆₄N₁₁O₆S₃: 1022.4203. found 1022.4192.

### Example 67: 6-[3-(1,3-benzothiazol-2-ylamino)-4-methyl-6,7-dihydro-5H-pyrido[2,3-c]pyridazin-8-yl]-3-[1-[[3-[2-[[6-[4-[2-(2,6-dioxo-3-piperidyl)-1-oxo-isoindolin-5-yl]-1-piperidyl]-6-oxo-hexyl]-methyl-amino]ethoxy]-5,7-dimethyl-1-adamantyl]methyl]-5-methyl-pyrazol-4-yl]pyridine-2-carboxylic acid

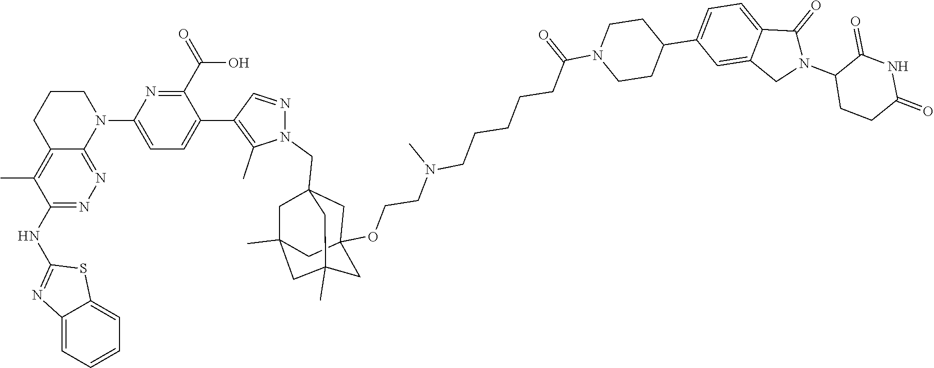

### Step A: 3-[5-[1-(6-bromohexanoyl)-4-piperidyl]-1-oxo-isoindolin-2-yl]piperidine-2,6-dione

Using General procedure for the acylation of piperidinyl-isoindolinone starting from 3-[1-oxo-5-(4-piperidyl)isoindolin-2-yl]piperidine-2,6-dione, hydrochloride (1:1) (0.42 mmol) and 6-bromohexanoic acid as the appropriate acid, 165 mg of the desired product were obtained. ¹H NMR (500 MHz, DMSO-d₆): δ ppm 10.98 (s, 1H), 7.65 (d, 1H), 7.49 (d, 1H), 7.40 (dd, 1H), 5.10 (dd, 1H), 4.57/4.00/3.12/2.60 (d+t/d+t, 4H), 4.42/4.28 (d+d, 2H), 3.54 (t, 2H), 2.91 (m, 1H), 2.91/2.59 (td+dd, 2H), 2.39/1.98 (dd+dt, 2H), 2.35 (t, 2H), 1.83/1.80/1.61/1.48 (dd+t/dd+t, 4H), 1.82 (qn, 2H), 1.54 (qn, 2H), 1.41 (qn, 2H); ¹³C NMR (125 MHz, DMSO-d₆) δ ppm 173.4, 171.5, 170.6, 168.4, 150.5, 143.0, 130.3, 127.5, 123.5, 122.3, 52.0, 47.6, 45.9/42.0, 42.6, 35.7, 33.9/33.2, 32.7, 32.6, 31.7, 27.9, 24.5, 23.0; HRMS-ESI (m/z): [M+H]⁺ calcd for C₂₄H₃₁BrN₃O₄: 504.1492. found: 504.1492.

### Step B: 6-[3-(1,3-benzothiazol-2-ylamino)-4-methyl-6,7-dihydro-5H-pyrido[2,3-c]pyridazin-8-yl]-3-[1-[[3-[2-[[6-[4-[2-(2,6-dioxo-3-piperidyl)-1-oxo-isoindolin-5-yl]-1-piperidyl]-6-oxo-hexyl]-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (30 mg) and the product of Step A as the appropriate alkylating agent, 9 mg of the desired product were obtained. HRMS-ESI (m/z): [M+H]⁺ calcd for C₆₅H₇₉N₁₂O₇S: 1171.5910. found: 1171.5906.

### Example 68: 6-[3-(1,3-benzothiazol-2-ylamino)-4-methyl-6,7-dihydro-5H-pyrido[2,3-c]pyridazin-8-yl]-3-[1-[[3-[2-[[8-[4-[2-(2,6-dioxo-3-piperidyl)-1-oxo-isoindolin-5-yl]-1-piperidyl]-8-oxo-octyl]-methyl-amino]ethoxy]-5,7-dimethyl-1-adamantyl]methyl]-5-methyl-pyrazol-4-yl]pyridine-2-carboxylic acid

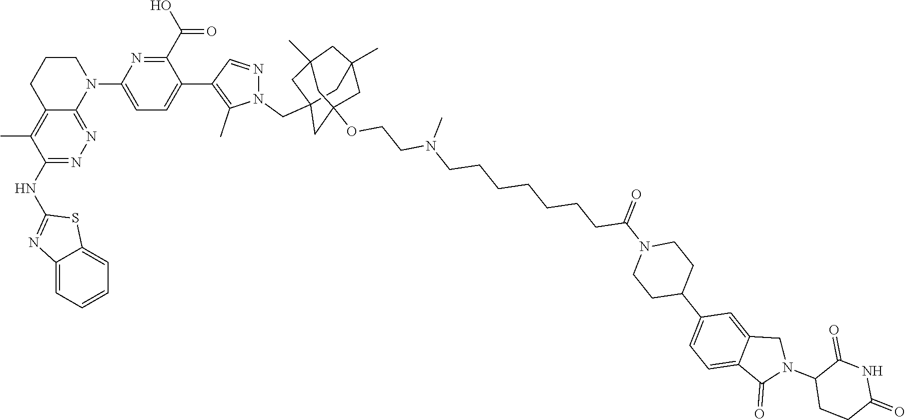 [SMILES: Cc1c(Nc2nc3ccccc3s2)nnc2c1CCCN2c1ccc(-c2cnn(CC34CC5(C)CC(C)(C3)CC(OCCN(C)CCCCCCCC(=O)N3CCC(c6ccc7c(c6)CN(C6CCC(=O)NC6=O)C7=O)CC3)(C5)C4)c2C)c(C(=O)O)n1]

### Step A: 3-[5-[1-(8-bromooctanoyl)-4-piperidyl]-1-oxo-isoindolin-2-yl]piperidine-2,6-dione

Using General procedure for the acylation of piperidinyl-isoindolinone starting from 3-[1-oxo-5-(4-piperidyl)isoindolin-2-yl]piperidine-2,6-dione, hydrochloride (1:1) (0.42 mmol) and 8-bromooctanoic acid as the appropriate acid, 220 mg of the desired product were obtained. ¹H NMR (500 MHz, DMSO-d₆): δ ppm 10.99 (s, 1H), 7.65 (d, 1H), 7.49 (d, 1H), 7.40 (dd, 1H), 5.10 (dd, 1H), 4.56/3.99/3.11/2.59 (m+m, 4H), 4.42/4.29 (d+d, 2H), 3.53 (t, 2H), 2.91 (m, 1H), 2.91/2.60 (m+m, 2H), 2.39/1.98 (m+m, 2H), 2.33 (t, 2H), 1.83/1.79/1.60/1.47 (m+m, 4H), 1.79 (m, 2H), 1.5 (m, 2H), 1.44-1.25 (m, 6H); ¹³C NMR (125 MHz, DMSO-d₆) δ ppm 127.4, 123.5, 122.2, 52.0, 47.6, 45.9/41.9, 42.6, 35.7, 33.9/33.1, 32.8, 32.7, 31.7, 25.3, 23.0; HRMS-ESI (m/z): [M+H]⁺ calcd for C₂₆H₃₅BrN₃O₄: 532.1805. found: 532.1806.

### Step B: 6-[3-(1,3-benzothiazol-2-ylamino)-4-methyl-6,7-dihydro-5H-pyrido[2,3-c]pyridazin-8-yl]-3-[1-[[3-[2-[[8-[4-[2-(2,6-dioxo-3-piperidyl)-1-oxo-isoindolin-5-yl]-1-piperidyl]-8-oxo-octyl]-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (30 mg) and the product of Step A as the appropriate alkylating agent, 13 mg of the desired product were obtained. HRMS-ESI (m/z): [M+2H]²⁺ calcd for C₇H₈₃N₁₂O₇S: 600.3148. found: 600.3153.

### Example 69: 6-[3-(1,3-benzothiazol-2-ylamino)-4-methyl-6,7-dihydro-5H-pyrido[2,3-c]pyridazin-8-yl]-3-[1-[[3-[2-[[12-[4-[2-(2,6-dioxo-3-piperidyl)-1-oxo-isoindolin-5-yl]-1-piperidyl]-12-oxo-dodecyl]-methyl-amino]ethoxy]-5,7-dimethyl-1-adamantyl]methyl]-5-methyl-pyrazol-4-yl]pyridine-2-carboxylic acid

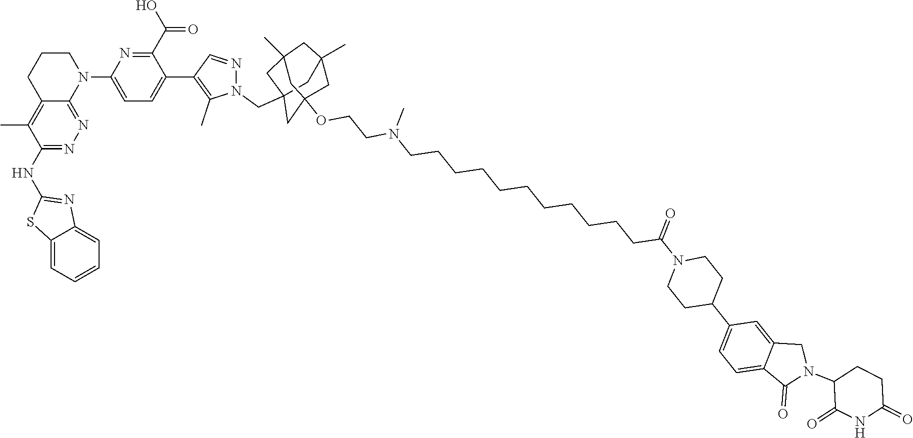 [SMILES: Cc1c(Nc2nc3ccccc3s2)nnc2c1CCCN2c1ccc(-c2cnn(CC34CC5(C)CC(C)(C3)CC(OCCN(C)CCCCCCCCCCCC(=O)N3CCC(c6ccc7c(c6)CN(C6CCC(=O)NC6=O)C7=O)CC3)(C5)C4)c2C)c(C(=O)O)n1]

### Step A: 3-[5-[1-(12-bromododecanoyl)-4-piperidyl]-1-oxo-isoindolin-2-yl]piperidine-2,6-dione

Using General procedure for the acylation of piperidinyl-isoindolinone starting from 3-[1-oxo-5-(4-piperidyl)isoindolin-2-yl]piperidine-2,6-dione, hydrochloride (1:1) (0.42 mmol) and 12-bromododecanoic acid as the appropriate acid, 199 mg of the desired product were obtained. ¹H NMR (500 MHz, DMSO-d₆): δ ppm 10.99 (s, 1H), 7.65 (d, 1H), 7.49 (d, 1H), 7.40 (dd, 1H), 5.10 (dd, 1H), 4.56/3.99/3.10/2.58 (m+m, 4H), 4.41/4.28 (d+d, 2H), 3.51 (t, 2H), 2.91 (m, 1H), 2.91/2.59 (m+m, 2H), 2.39/1.99 (m+m, 2H), 2.32 (t, 2H), 1.82/1.78/1.59/1.47 (m+m, 4H), 1.78 (m, 2H), 1.50 (m, 2H), 1.41-1.20 (m, 14H); ¹³C NMR (125 MHz, DMSO-d₆) δ ppm 127.4, 123.5, 122.2, 52.0, 47.6, 45.9/41.9, 42.6, 35.7, 33.9/33.2, 32.9, 32.7, 31.7, 25.4, 23.0; HRMS-ESI (m/z): [M+H]⁺ calcd for C₃₀H₄₃BrN₃O₄: 588.2431. found: 588.2432.

### Step B: 6-[3-(1,3-benzothiazol-2-ylamino)-4-methyl-6,7-dihydro-5H-pyrido[2,3-c]pyridazin-8-yl]-3-[1-[[3-[2-[[12-[4-[2-(2,6-dioxo-3-piperidyl)-1-oxo-isoindolin-5-yl]-1-piperidyl]-12-oxo-dodecyl]-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (30 mg) and the product of Step A as the appropriate alkylating agent, 17 mg of the desired product were obtained. HRMS-ESI (m/z): [M+2H]²⁺ calcd for C₇1H₉₂N₁₂07S: 628.3466. found: 628.3463.

### Example 70: 6-[3-(1,3-benzothiazol-2-ylamino)-4-methyl-6,7-dihydro-5H-pyrido[2,3-c]pyridazin-8-yl]-3-[1-[[3-[2-[[10-[4-[2-(2,6-dioxo-3-piperidyl)-1-oxo-isoindolin-5-yl]-1-piperidyl]-10-oxo-decyl]-methyl-amino]ethoxy]-5,7-dimethyl-1-adamantyl]methyl]-5-methyl-pyrazol-4-yl]pyridine-2-carboxylic acid

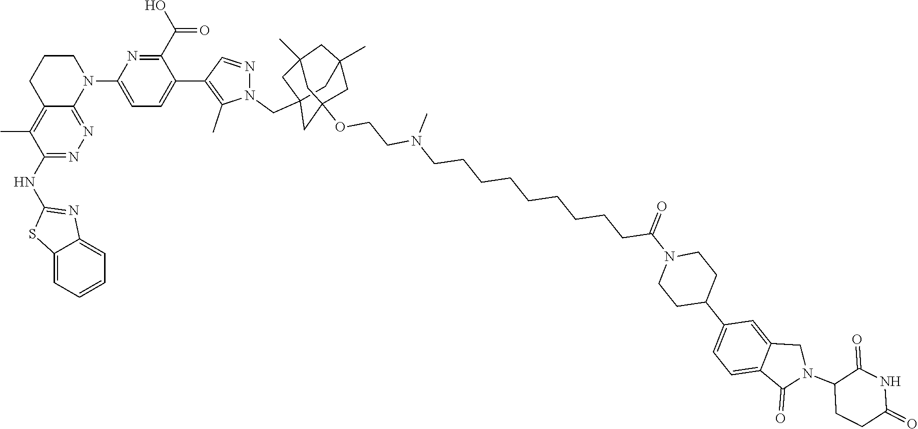 [SMILES: Cc1c(Nc2nc3ccccc3s2)nnc2c1CCCN2c1ccc(-c2cnn(CC34CC5(C)CC(C)(C3)CC(OCCN(C)CCCCCCCCCC(=O)N3CCC(c6ccc7c(c6)CN(C6CCC(=O)NC6=O)C7=O)CC3)(C5)C4)c2C)c(C(=O)O)n1]

### Step A: 3-[5-[1-(10-bromodecanoyl)-4-piperidyl]-1-oxo-isoindolin-2-yl]piperidine-2,6-dione

Using General procedure for the acylation of piperidinyl-isoindolinone starting from 3-[1-oxo-5-(4-piperidyl)isoindolin-2-yl]piperidine-2,6-dione, hydrochloride (1:1) (0.42 mmol) and 10-bromodecanoic acid as the appropriate acid, 71 mg of the desired product were obtained. ¹H NMR (500 MHz, DMSO-d₆): δ ppm 10.99 (s, 1H), 7.65 (brd, 1H), 7.49 (d, 1H), 7.39 (dd, 1H), 5.10 (dd, 1H), 4.56/3.99/3.11/2.91 (t+t/t+t, 4H), 4.42/4.28 (d+d, 2H), 3.52 (t, 2H), 2.91/2.59 (td+dd, 2H), 2.91 (t, 1H), 2.40/1.99 (dd+dt, 2H), 2.32 (t, 2H), 1.78 (qn, 2H), 1.50 (qn, 2H), 1.40-1.25 (m, 10H), 1.28 (qn, 2H), 1.27 (qn, 2H); ¹³C NMR (125 MHz, DMSO-d₆) δ ppm 127.4, 123.5, 122.2, 52.0, 47.6, 46.0/42.0, 42.7, 35.7, 33.1, 32.7, 31.7, 29.3, 28.0, 25.4, 23.0; HRMS-ESI (m/z): [M+H]⁺ calcd for C₂₈H₃₉BrN₃O₄: 560.2118. found: 560.2121.

### Step B: 6-[3-(1,3-benzothiazol-2-ylamino)-4-methyl-6,7-dihydro-5H-pyrido[2,3-c]pyridazin-8-yl]-3-[1-[[3-[2-[[10-[4-[2-(2,6-dioxo-3-piperidyl)-1-oxo-isoindolin-5-yl]-1-piperidyl]-10-oxo-decyl]-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (30 mg) and the product of Step A as the appropriate alkylating agent, 14 mg of the desired product were obtained. HRMS-ESI (m/z): [M+H]⁺ calcd for C₆₉H₈₇N₁₂O₇S: 1227.6536. found: 1227.6538.

### Example 71: 6-[3-(1,3-benzothiazol-2-ylamino)-4-methyl-6,7-dihydro-5H-pyrido[2,3-c]pyridazin-8-yl]-3-[1-[[3-[2-[[9-[4-[2-(2,6-dioxo-3-piperidyl)-1-oxo-isoindolin-5-yl]-1-piperidyl]-9-oxo-nonyl]-methyl-amino]ethoxy]-5,7-dimethyl-1-adamantyl]methyl]-5-methyl-pyrazol-4-yl]pyridine-2-carboxylic acid

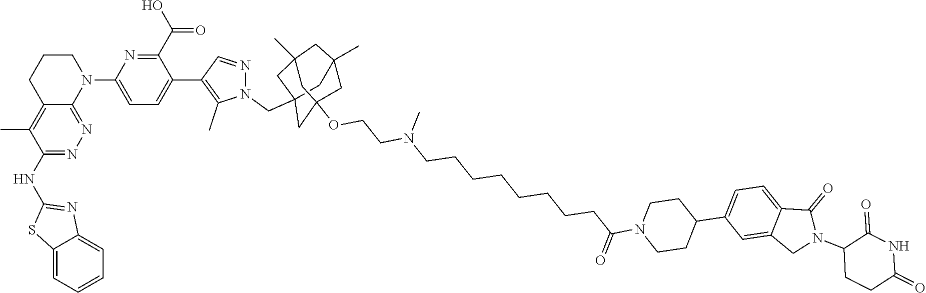 [SMILES: Cc1c(Nc2nc3ccccc3s2)nnc2c1CCCN2c1ccc(-c2cnn(CC34CC5(C)CC(C)(C3)CC(OCCN(C)CCCCCCCCC(=O)N3CCC(c6ccc7c(c6)CN(C6CCC(=O)NC6=O)C7=O)CC3)(C5)C4)c2C)c(C(=O)O)n1]

### Step A: 3-[5-[1-(9-bromononanoyl)-4-piperidyl]-1-oxo-isoindolin-2-yl]piperidine-2,6-dione

Using General procedure for the acylation of piperidinyl-isoindolinone starting from 3-[1-oxo-5-(4-piperidyl)isoindolin-2-yl]piperidine-2,6-dione, hydrochloride (1:1) (0.42 mmol) and 9-bromononanoic acid as the appropriate acid, 121 mg of the desired product were obtained. ¹H NMR (500 MHz, DMSO-d₆): δ ppm 10.99 (s, 1H), 7.65 (d, 1H), 7.49 (d, 1H), 7.40 (dd, 1H), 5.10 (dd, 1H), 4.56/3.99/3.10/2.59 (d+dd/d+dd, 4H), 4.42/4.28 (d+d, 2H), 3.52 (t, 2H), 2.91/2.60 (td+dd, 2H), 2.91 (t, 1H), 2.39/1.99 (dd+dt, 2H), 2.33 (t, 2H), 1.83/1.79/1.59/1.49 (d+dd/d+dd, 4H), 1.79 (qn, 2H), 1.50 (qn, 2H), 1.38 (qn, 2H), 1.32-1.26 (m, 6H); ¹³C NMR (125 MHz, DMSO-d₆) δ ppm 173.4, 171.6, 170.9, 168.4, 150.5, 143.0, 130.4, 127.4, 123.5, 122.2, 52.0, 47.6, 45.9/41.9, 42.6, 35.7, 33.9/33.1, 32.9, 32.7, 31.7, 28.0, 25.4, 23.0; HRMS-ESI (m/z): [M+H]⁺ calcd for C₂₇H₃₇BrN₃O₄: 546.1962. found: 546.1960.

### Step B: 6-[3-(1,3-benzothiazol-2-ylamino)-4-methyl-6,7-dihydro-5H-pyrido[2,3-c]pyridazin-8-yl]-3-[1-[[3-[2-[[9-[4-[2-(2,6-dioxo-3-piperidyl)-1-oxo-isoindolin-5-yl]-1-piperidyl]-9-oxo-nonyl]-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (30 mg) and the product of Step A as the appropriate alkylating agent, 18 mg of the desired product were obtained. HRMS-ESI (m/z): [M+H]⁺ calcd for C₆₈H₈₅N₁₂O₇S: 1213.6379. found: 1213.6374.

### Example 72: 6-[3-(1,3-benzothiazol-2-ylamino)-4-methyl-6,7-dihydro-5H-pyrido[2,3-c]pyridazin-8-yl]-3-[1-[[3-[2-[9-[2-[[[(2S,4R)-1-[(2S)-2-[(1-fluorocyclopropanecarbonyl) amino]-3,3-dimethyl-butanoyl]-4-hydroxy-pyrrolidine-2-carbonyl]amino]methyl]-5-(4-methylthiazol-5-yl)phenoxy]nonyl-methyl-amino]ethoxy]-5,7-dimethyl-1-adamantyl]methyl]-5-methyl-pyrazol-4-yl]pyridine-2-carboxylic acid

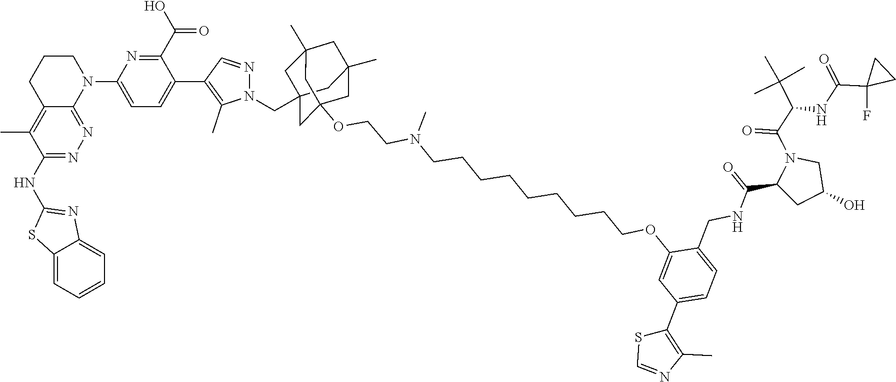 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)c1]

### Step A: (2S,4R)—N-[[2-(9-bromononyl)-4-(4-methylthiazol-5-yl)phenyl]methyl]-1-[(2S)-2-[(1-fluorocyclopropanecarbonyl)amino]-3,3-dimethyl-butanoyl]-4-hydroxy-pyrrolidine-2-carboxamide

Using General procedure for the alkylation of VHL ligand on hydroxy group starting from (2S,4R)-1-[(2S)-2-[(1-fluorocyclopropanecarbonyl)amino]-3,3-dimethyl-butanoyl]-4-hydroxy-N-[[2-hydroxy-4-(4-methylthiazol-5-yl)phenyl]methyl]pyrrolidine-2-carboxamide (0.38 mmol) and 1,9-dibromononane as the appropriate reactant, 147 mg of the desired product were obtained. ¹H NMR (500 MHz, DMSO-d₆): δ ppm 8.98 (s, 1H), 8.50 (t, 1H), 7.40 (d, 1H), 7.29 (dd, 1H), 6.99 (d, 1H), 6.94 (dd, 1H), 5.17 (br., 1H), 4.60 (d, 1H), 4.51 (t, 1H), 4.35 (br., 1H), 4.28/4.19 (dd+dd, 2H), 4.04 (t, 2H), 3.65/3.60 (dd+d, 2H), 3.51 (t, 2H), 2.45 (s, 3H), 2.08/1.92 (m+m, 2H), 1.84-1.19 (m, 18H), 1.37/1.22 (m+m, 4H), 0.96 (s, 9H); ¹³C NMR (125 MHz, DMSO-d₆) δ ppm 172.3, 169.4, 168.5, 151.9, 128.1, 121.1, 112, 78.6, 69.4, 68.1, 59.3, 57.2, 57.0, 38.4, 37.7, 36.5, 35.7, 26.6, 16.5, 13.3; HRMS-ESI (m/z): [M+H]⁺ calcd for C₃₅H₅₁BrFN₄O₅S: 737.2742. found: 737.2743.

### Step B: 6-[3-(1,3-benzothiazol-2-ylamino)-4-methyl-6,7-dihydro-5H-pyrido[2,3-c]pyridazin-8-yl]-3-[1-[[3-[2-[9-[2-[[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]nonyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (30 mg) and the product of Step A as the appropriate alkylating agent, 21 mg of the desired product were obtained. HRMS-ESI (m/z): [M+2H]²⁺ calcd for C₇₆H₁₀₀FN₁₃O₈S₂: 702.8616. found: 702.8611.

### Example 73: 6-[3-(1,3-benzothiazol-2-ylamino)-4-methyl-6,7-dihydro-5H-pyrido[2,3-c]pyridazin-8-yl]-3-[1-[[3-[2-[10-[2-[[[(2S,4R)-1-[(2S)-2-[(1-fluorocyclopropanecarbonyl) amino]-3,3-dimethyl-butanoyl]-4-hydroxy-pyrrolidine-2-carbonyl]amino]methyl]-5-(4-methylthiazol-5-yl)phenoxy]decyl-methyl-amino]ethoxy]-5,7-dimethyl-1-adamantyl]methyl]-5-methyl-pyrazol-4-yl]pyridine-2-carboxylic acid

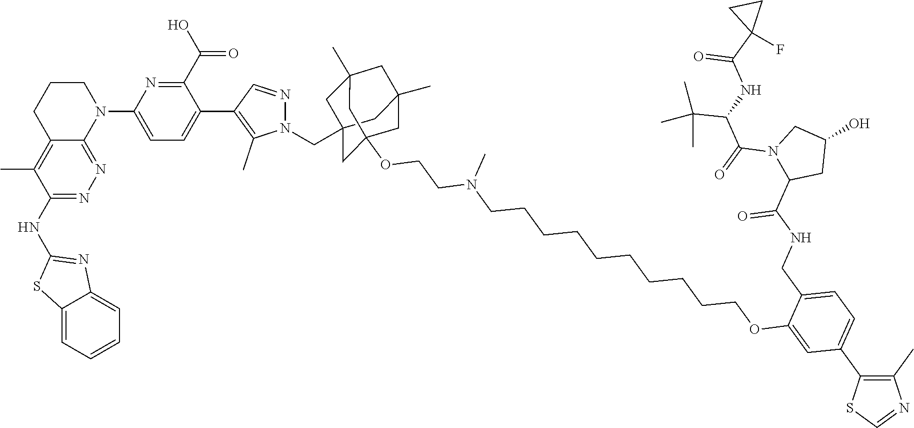

### Step A: (2S,4R)—N-[[2-(10-bromodecoxy)-4-(4-methylthiazol-5-yl)phenyl]methyl]-1-[(2S)-2-[(1-fluorocyclopropanecarbonyl)amino]-3,3-dimethyl-butanoyl]-4-hydroxy-pyrrolidine-2-carboxamide

Using General procedure for the alkylation of VHL ligand on hydroxy group starting from (2S,4R)-1-[(2S)-2-[(1-fluorocyclopropanecarbonyl)amino]-3,3-dimethyl-butanoyl]-4-hydroxy-N-[[2-hydroxy-4-(4-methylthiazol-5-yl)phenyl]methyl]pyrrolidine-2-carboxamide (0.38 mmol) and 1,10-dibromodecane as the appropriate reactant, 134 mg of the desired product were obtained. ¹H NMR (500 MHz, DMSO-d₆): δ ppm 8.98 (s, 1H), 8.50 (t, 1H), 7.40 (d, 1H), 7.29 (dd, 1H), 6.99 (d, 1H), 6.94 (dd, 1H), 5.18 (d, 1H), 4.60 (d, 1H), 4.51 (t, 1H), 4.35 (br., 1H), 4.29/4.19 (dd+dd, 2H), 4.04 (t, 2H), 3.65/3.59 (dd+d, 2H), 3.51 (t, 2H), 2.45 (s, 3H), 2.09/1.92 (m+m, 2H), 1.78-1.20 (m, 16H), 1.36/1.23 (m+m, 4H), 0.96 (s, 9H); ¹³C NMR (125 MHz, DMSO-d₆) δ ppm 172.3, 169.4, 168.5, 151.9, 128.1, 121.1, 112.0, 78.6, 69.4, 68.1, 59.3, 57.2, 57.0, 38.4, 37.7, 36.5, 35.7, 26.6, 16.5, 13.3; HRMS-ESI (m/z): [M+H]⁺ calcd for C₃₆H₅₃BrFN₄O₅S: 751.2898. found: 751.2897.

### Step B: 6-[3-(1,3-benzothiazol-2-ylamino)-4-methyl-6,7-dihydro-5H-pyrido[2,3-c]pyridazin-8-yl]-3-[1-[[3-[2-[10-[2-[[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]decyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (30 mg) and the product of Step A as the appropriate alkylating agent, 25 mg of the desired product were obtained. HRMS-ESI (m/z): [M+2H]²⁺ calcd for C₇₇H₁₀₂FN₁₃O₈S₂: 709.8695. found: 709.8696.

### Example 74: 6-[3-(1,3-benzothiazol-2-ylamino)-4-methyl-6,7-dihydro-5H-pyrido[2,3-c]pyridazin-8-yl]-3-[1-[[3-[2-[[16-[4-[2-(2,6-dioxo-3-piperidyl)-1-oxo-isoindolin-5-yl]-1-piperidyl]-16-oxo-hexadecyl]-methyl-amino]ethoxy]-5,7-dimethyl-1-adamantyl]methyl]-5-methyl-pyrazol-4-yl]pyridine-2-carboxylic acid

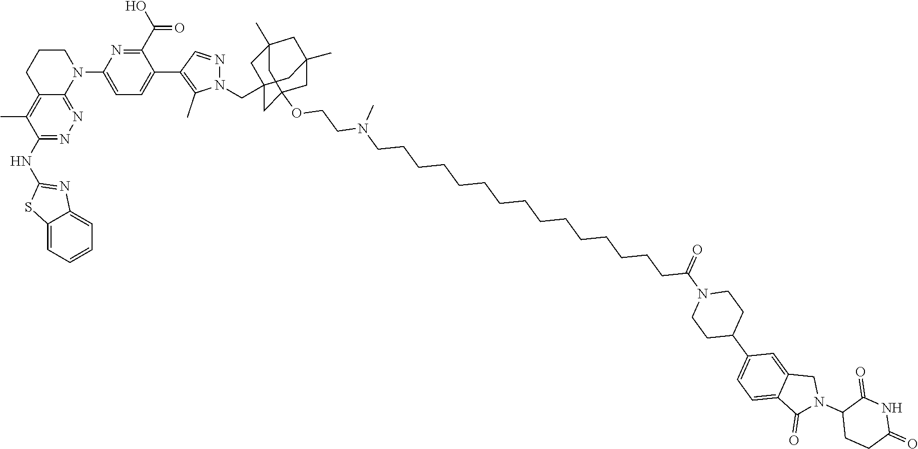 [SMILES: Cc1c(Nc2nc3ccccc3s2)nnc2c1CCCN2c1ccc(-c2cnn(CC34CC5(C)CC(C)(C3)CC(OCCN(C)CCCCCCCCCCCCCCCC(=O)N3CCC(c6ccc7c(c6)CN(C6CCC(=O)NC6=O)C7=O)CC3)(C5)C4)c2C)c(C(=O)O)n1]

### Step A: 3-[5-[1-(16-bromohexadecanoyl)-4-piperidyl]-1-oxo-isoindolin-2-yl]piperidine-2,6-dione

Using General procedure for the acylation of piperidinyl-isoindolinone starting from 3-[1-oxo-5-(4-piperidyl)isoindolin-2-yl]piperidine-2,6-dione, hydrochloride (1:1) (0.42 mmol) and 16-bromohexadecanoic acid as the appropriate acid, 244 mg of the desired product were obtained. ¹H NMR (500 MHz, DMSO-d₆): δ ppm 10.98 (s, 1H), 7.65 (d, 1H), 7.49 (brs., 1H), 7.39 (d, 1H), 5.10 (dd, 1H), 4.56/3.99/3.10/2.59 (d+m/d+m, 4H), 4.42/4.28 (d+d, 2H), 2.91 (m, 1H), 2.91/2.59 (m+m, 2H), 2.39/1.98 (m+m, 2H), 2.32 (t, 2H), 1.90-1.30 (m, 32H); ¹³C NMR (125 MHz, DMSO-d₆) δ ppm 173.4/171.6, 170.9, 168.5, 127.4, 123.5, 122.2, 52.0, 47.6, 45.9/41.9, 42.6, 32.9, 31.7, 23.0; HRMS-ESI (m/z): [M+H]⁺ calcd for C₃₄H₅₁BrN₃O₄: 644.3057. found: 644.3058.

### Step B: 6-[3-(1,3-benzothiazol-2-ylamino)-4-methyl-6,7-dihydro-5H-pyrido[2,3-c]pyridazin-8-yl]-3-[1-[[3-[2-[[16-[4-[2-(2,6-dioxo-3-piperidyl)-1-oxo-isoindolin-5-yl]-1-piperidyl]-16-oxo-hexadecyl]-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (30 mg) and the product of Step A as the appropriate alkylating agent, 8 mg of the desired product were obtained. HRMS-ESI (m/z): [M+2H]2+ calcd for C₇₅H₁₀₀N₁₂O₇S: 656.3774. found: 656.3774.

### Example 75: 6-[3-(1,3-benzothiazol-2-ylamino)-4-methyl-6,7-dihydro-5H-pyrido[2,3-c]pyridazin-8-yl]-3-[1-[[3-[2-[[11-[4-[2-(2,6-dioxo-3-piperidyl)-1-oxo-isoindolin-5-yl]-1-piperidyl]-11-oxo-undecyl]-methyl-amino]ethoxy]-5,7-dimethyl-1-adamantyl]methyl]-5-methyl-pyrazol-4-yl]pyridine-2-carboxylic acid

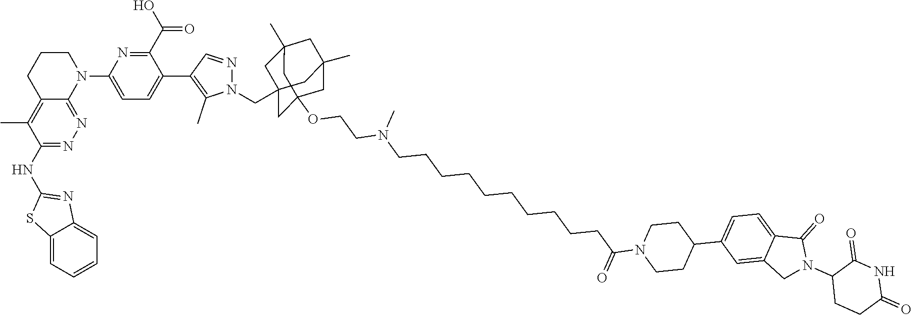 [SMILES: Cc1c(Nc2nc3ccccc3s2)nnc2c1CCCN2c1ccc(-c2cnn(CC34CC5(C)CC(C)(C3)CC(OCCN(C)CCCCCCCCCCC(=O)N3CCC(c6ccc7c(c6)CN(C6CCC(=O)NC6=O)C7=O)CC3)(C5)C4)c2C)c(C(=O)O)n1]

### Step A: 3-[5-[1-(11-bromoundecanoyl)-4-piperidyl]-1-oxo-isoindolin-2-yl]piperidine-2,6-dione

Using General procedure for the acylation of piperidinyl-isoindolinone starting from 3-[1-oxo-5-(4-piperidyl)isoindolin-2-yl]piperidine-2,6-dione, hydrochloride (1:1) (0.42 mmol) and 11-bromoundecanoic acid as the appropriate acid, 176 mg of the desired product were obtained. HRMS-ESI (m/z): [M+H]⁺ calcd for C₂₉H₄₁BrN₃O₄: 574.2275. found: 574.2273.

### Step B: 6-[3-(1,3-benzothiazol-2-ylamino)-4-methyl-6,7-dihydro-5H-pyrido[2,3-c]pyridazin-8-yl]-3-[1-[[3-[2-[[11-[4-[2-(2,6-dioxo-3-piperidyl)-1-oxo-isoindolin-5-yl]-1-piperidyl]-11-oxo-undecyl]-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (30 mg) and the product of Step A as the appropriate alkylating agent, 10 mg of the desired product were obtained. HRMS-ESI (m/z): [M+2H]²⁺ calcd for C₇₀H₉₀N₁₂O₇S: 621.3383. found: 621.3386.

### Example 76: 6-[3-(1,3-benzothiazol-2-ylamino)-4-methyl-6,7-dihydro-5H-pyrido[2,3-c]pyridazin-8-yl]-3-[1-[[3-[2-[11-[2-[[[(2S,4R)-1-[(2S)-2-[(1-fluorocyclopropanecarbonyl) amino]-3,3-dimethyl-butanoyl]-4-hydroxy-pyrrolidine-2-carbonyl]amino]methyl]-5-(4-methylthiazol-5-yl)phenoxy]undecyl-methyl-amino]ethoxy]-5,7-dimethyl-1-adamantyl]methyl]-5-methyl-pyrazol-4-yl]pyridine-2-carboxylic acid

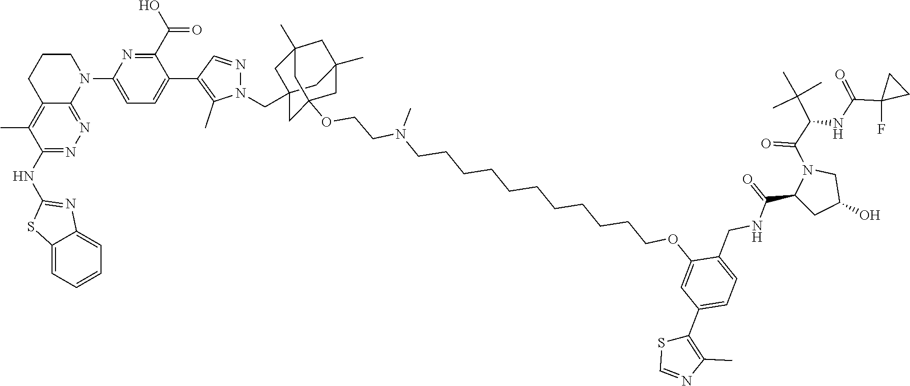 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)c1]

### Step A: (2S,4R)—N-[[2-(11-bromoundecoxy)-4-(4-methylthiazol-5-yl)phenyl]methyl]-1-[(2S)-2-[(1-fluorocyclopropanecarbonyl)amino]-3,3-dimethyl-butanoyl]-4-hydroxy-pyrrolidine-2-carboxamide

Using General procedure for the alkylation of VHL ligand on hydroxy group starting from (2S,4R)-1-[(2S)-2-[(1-fluorocyclopropanecarbonyl)amino]-3,3-dimethyl-butanoyl]-4-hydroxy-N-[[2-hydroxy-4-(4-methylthiazol-5-yl)phenyl]methyl]pyrrolidine-2-carboxamide (0.28 mmol) and 1,11-dibromoundecane as the appropriate reactant, 45 mg of the desired product were obtained. ¹H NMR (500 MHz, DMSO-de): δ ppm 8.98 (s, 1H), 8.50 (t, 1H), 7.40 (d, 1H), 7.29 (dd, 1H), 6.99 (d, 1H), 6.94 (dd, 1H), 5.17 (d, 1H), 4.60 (d, 1H), 4.51 (t, 1H), 4.35 (br., 1H), 4.28/4.19 (dd+dd, 2H), 4.04 (t, 2H), 3.65/3.6 (dd+d, 2H), 3.51 (t, 2H), 2.45 (s, 3H), 2.08/1.92 (m+m, 2H), 1.82-1.21 (m, 18H), 1.37/1.22 (m+m, 4H), 0.96 (s, 9H); 130 NMR (125 MHz, DMSO-d₆) δ ppm 172.3, 169.4, 168.5, 151.9, 128.1, 121.1, 112.0, 78.6, 69.4, 68.1, 59.3, 57.2, 57.0, 38.4, 37.7, 36.5, 35.7, 26.6, 16.5, 13.3; HRMS-ESI (m/z): [M+H]⁺ calcd for C₃₇H₅₅BrFN₄O₅S: 765.3055. found: 765.3059.

### Step B: 6-[3-(1,3-benzothiazol-2-ylamino)-4-methyl-6,7-dihydro-5H-pyrido[2,3-c]pyridazin-8-yl]-3-[1-[[3-[2-[11-[2-[[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]undecyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (30 mg) and the product of Step A as the appropriate alkylating agent, 19 mg of the desired product were obtained. HRMS-ESI (m/z): [M+2H]²⁺ calcd for C₇₈H₁₀₄FN₁₃O₈S₂: 716.8773. found: 716.8770.

### Example 77: 6-[3-(1,3-benzothiazol-2-ylamino)-4-methyl-6,7-dihydro-5H-pyrido[2,3-c]pyridazin-8-yl]-3-[1-[[3-[2-[16-[2-[[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]hexadecyl-methyl-amino]ethoxy]-5,7-dimethyl-1-adamantyl]methyl]-5-methyl-pyrazol-4-yl]pyridine-2-carboxylic acid

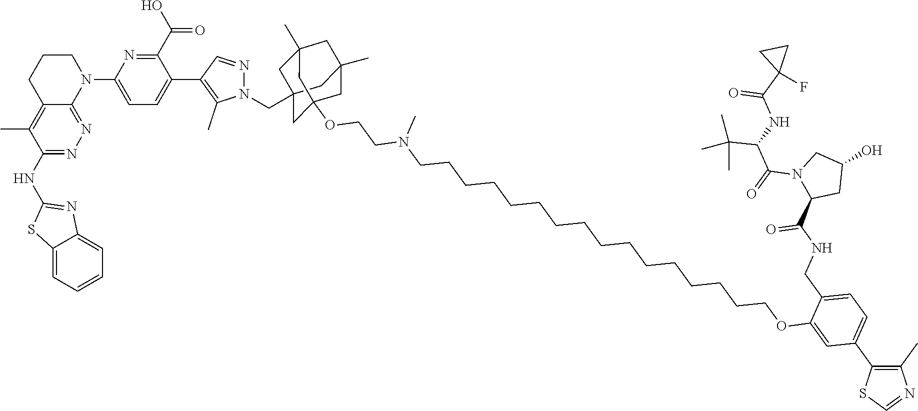

### Step A: (2S,4R)—N-[[2-(16-bromohexadecoxy)-4-(4-methylthiazol-5-yl)phenyl]methyl]-1-[(2S)-2-[(1-fluorocyclopropanecarbonyl)amino]-3,3-dimethyl-butanoyl]-4-hydroxy-pyrrolidine-2-carboxamide

Using General procedure for the alkylation of VHL ligand on hydroxy group starting from (2S,4R)-1-[(2S)-2-[(1-fluorocyclopropanecarbonyl)amino]-3,3-dimethyl-butanoyl]-4-hydroxy-N-[[2-hydroxy-4-(4-methylthiazol-5-yl)phenyl]methyl]pyrrolidine-2-carboxamide (0.28 mmol) and 1,16-dibromohexadecane as the appropriate reactant, 40 mg of the desired product were obtained. HRMS-ESI (m/z): [M+H]⁺ calcd for C₄₂H₆₅BrFN₄O₅S: 835.3838. found: 835.3835.

### Step B: 6-[3-(1,3-benzothiazol-2-ylamino)-4-methyl-6,7-dihydro-5H-pyrido[2,3-c]pyridazin-8-yl]-3-[1-[[3-[2-[16-[2-[[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]hexadecyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (20 mg) and the product of Step A as the appropriate alkylating agent, 10 mg of the desired product were obtained. HRMS-ESI (m/z): [M+2H]²⁺ calcd for C₈₃H₁₁₄FN₁₃O₈S₂: 751.9164. found: 751.9165.

### Example 78: 6-[3-(1,3-benzothiazol-2-ylamino)-4-methyl-6,7-dihydro-5H-pyrido[2,3-c]pyridazin-8-yl]-3-[1-[[3-[2-[[13-[4-[2-(2,6-dioxo-3-piperidyl)-1-oxo-isoindolin-5-yl]-1-piperidyl]-13-oxo-tridecyl]-methyl-amino]ethoxy]-5,7-dimethyl-1-adamantyl]methyl]-5-methyl-pyrazol-4-yl]pyridine-2-carboxylic acid

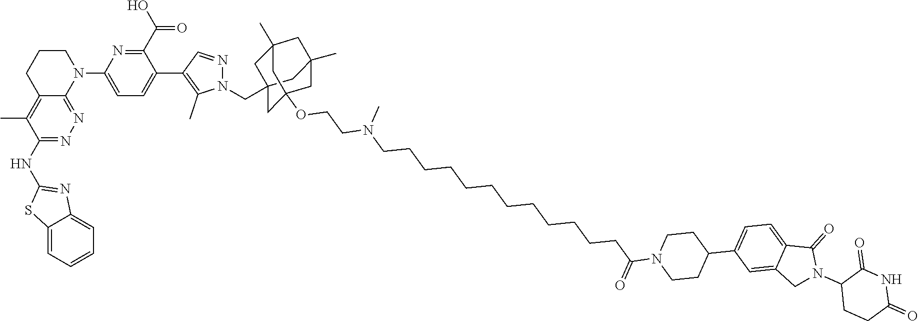 [SMILES: Cc1c(Nc2nc3ccccc3s2)nnc2c1CCCN2c1ccc(-c2cnn(CC34CC5(C)CC(C)(C3)CC(OCCN(C)CCCCCCCCCCCCC(=O)N3CCC(c6ccc7c(c6)CN(C6CCC(=O)NC6=O)C7=O)CC3)(C5)C4)c2C)c(C(=O)O)n1]

### Step A: 3-[5-[1-(13-hydroxytridecanoyl)-4-piperidyl]-1-oxo-isoindoline-2-yl]piperidine-2,6-dione

Using General procedure for the acylation of piperidinyl-isoindolinone starting from 3-[1-oxo-5-(4-piperidyl)isoindolin-2-yl]piperidine-2,6-dione, hydrochloride (1:1) (0.63 mmol) and 13-hydroxytridecanoic acid as the appropriate acid, 265 mg of the desired product were obtained. ¹H NMR (500 MHz, DMSO-d₆): δ ppm 10.98 (s, 1H), 7.65 (d, 1H), 7.49 (brs., 1H), 7.40 (d, 1H), 5.10 (dd, 1H), 4.57/3.99/3.10/2.59 (d+m/br+br., 4H), 4.42/4.28 (d+d, 2H), 3.36 (m, 2H), 2.91 (m, 1H), 2.91/2.59 (m+m, 2H), 2.39/1.99 (m+m, 2H), 2.32 (t, 2H), 1.88-1.44 (m, 4H), 1.55-1.20 (m, 20H); ¹³C NMR (125 MHz, DMSO-d₆) δ ppm 173.4, 171.6, 170.9, 168.5, 127.4, 123.5, 122.3, 61.2, 52.0, 47.6, 45.9/41.9, 42.6, 33.9/33.2, 32.9, 31.7, 23.0; HRMS-ESI (m/z): [M+H]⁺ calcd for C₃₁H₄₆N₃O₅: 540.3432. found: 540.3438.

### Step B: [13-[4-[2-(2,6-dioxo-3-piperidyl)-1-oxo-isoindolin-5-yl]-1-piperidyl]-13-oxo-tridecyl] 4-methylbenzenesulfonate

Using General procedure for the tosylation of the hydroxyalkyl VHL ligand-derivatives starting from product of Step A (0.32 mmol), 56 mg of the desired product were obtained. ¹H NMR (500 MHz, DMSO-d₆): δ ppm 10.98 (s, 1H), 7.78 (dm, 2H), 7.65 (d, 1H), 7.48 (brs., 1H), 7.47 (dm, 2H), 7.39 (d, 1H), 5.10 (dd, 1H), 4.61-1.03 (m, 32H), 4.41/4.28 (d+d, 2H), 2.91 (tm, 1H), 2.91/2.59 (m+m, 2H), 2.42 (s, 3H), 2.39/1.98 (m+m, 2H); ¹³C NMR (125 MHz, DMSO-d₆) δ ppm 173.4/171.6, 170.9, 168.5, 130.6, 128.0, 127.4, 123.5, 122.2, 52.0, 47.6, 42.6, 31.7, 23.0, 21.6; HRMS-ESI (m/z): [M+H]⁺ calcd for C₃₈H₅₂N₃O₇S: 694.3520. found: 694.3522.

### Step C: 6-[3-(1,3-benzothiazol-2-ylamino)-4-methyl-6,7-dihydro-5H-pyrido[2,3-c]pyridazin-8-yl]-3-[1-[[3-[2-[[13-[4-[2-(2,6-dioxo-3-piperidyl)-1-oxo-isoindolin-5-yl]-1-piperidyl]-13-oxo-tridecyl]-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (25 mg) and the product of Step B as the appropriate alkylating agent, 4 mg of the desired product were obtained. HRMS-ESI (m/z): [M+2H]²⁺ calcd for C₇₂H₉₄N₁₂07S: 635.3539. found: 635.3533.

### Example 79: 6-[3-(1,3-benzothiazol-2-ylamino)-4-methyl-6,7-dihydro-5H-pyrido[2,3-c]pyridazin-8-yl]-3-[1-[[3-[2-[12-[(2R)-2-[(1-fluorocyclopropanecarbonyl)amino]-3-[(2S,4R)-4-hydroxy-2-[[4-(4-methylthiazol-5-yl)phenyl]methylcarbamoyl]pyrrolidin-1-yl]-1,1-dimethyl-3-oxo-propyl]sulfanyldodecyl-methyl-amino]ethoxy]-5,7-dimethyl-1-adamantyl]methyl]-5-methyl-pyrazol-4-yl]pyridine-2-carboxylic acid

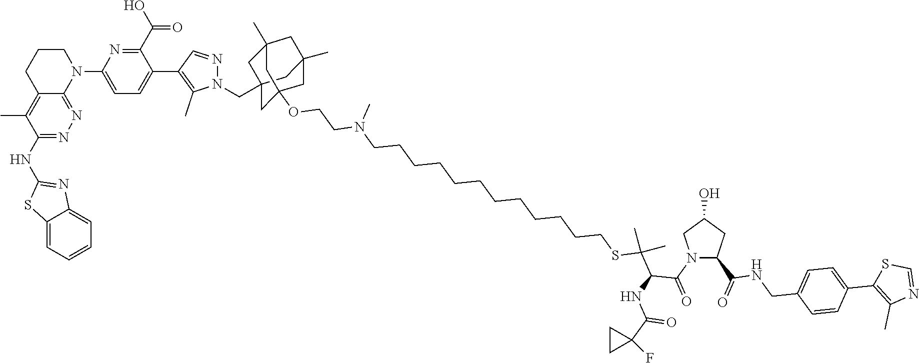 [SMILES: Cc1ncsc1-c1ccc(CNC(=O)[C@@H]2C[C@@H](O)CN2C(=O)[C@@H](NC(=O)C2(F)CC2)C(C)(C)SCCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)cc1]

### Step A: (2S,4R)-1-[(2R)-3-(12-bromododecylsulfanyl)-2-[(1-fluorocyclopropanecarbonyl) amino]-3-methyl-butanoyl]-4-hydroxy-N-[[4-(4-methylthiazol-5-yl)phenyl]methyl] pyrrolidine-2-carboxamide

Using General procedure for the alkylation of VHL ligand on thiol group starting from (2S,4R)-1-[(2R)-2-[(1-fluorocyclopropanecarbonyl)amino]-3-methyl-3-sulfanyl-butanoyl]-4-hydroxy-N-[[4-(4-methylthiazol-5-yl)phenyl]methyl]pyrrolidine-2-carboxamide (0.09 mmol) and 1,12-dibromododecane as the appropriate reactant, 53 mg of the desired product were obtained. ¹H NMR (500 MHz, DMSO-d₆): δ ppm 8.98 (s, 1H), 8.58 (t, 1H), 7.48 (dd, 1H), 7.42 (dm, 2H), 7.39 (dm, 2H), 5.19 (d, 1H), 4.78 (d, 1H), 4.46 (t, 1H), 4.43/4.23 (dd+dd, 2H), 4.36 (br., 1H), 3.72/3.64 (dd+dd, 2H), 3.51 (t, 2H), 2.51 (m, 2H), 2.45 (s, 3H), 2.08/1.91 (m+m, 2H), 1.77 (m, 2H), 1.42-1.13 (m, 18H), 1.38/1.35 (s+s, 6H), 1.38/1.21 (m+m, 4H); ¹³C NMR (125 MHz, DMSO-d₆) δ ppm 172.0, 168.6, 168.3, 151.9, 129.2, 127.9, 78.5, 69.3, 59.5, 57.0, 55.3, 49.5, 42.1, 38.4, 35.7, 32.7, 28.2, 27.2/24.7, 16.4, 13.4; HRMS-ESI (m/z): [M+H]⁺ calcd for C₃₇H₅₅BrFN₄O₄S₂: 781.2827. found: 781.2824.

### Step B: 6-[3-(1,3-benzothiazol-2-ylamino)-4-methyl-6,7-dihydro-5H-pyrido[2,3-c]pyridazin-8-yl]-3-[1-[[3-[2-[12-[(2R)-2-[(1-fluorocyclopropanecarbonyl)amino]-3-[(2S,4R)-4-hydroxy-2-[[4-(4-methylthiazol-5-yl)phenyl]methylcarbamoyl]pyrrolidin-1-yl]-1,1-dimethyl-3-oxo-propyl]sulfanyldodecyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (30 mg) and the product of Step A as the appropriate alkylating agent, 22 mg of the desired product were obtained. HRMS-ESI (m/z): [M+2H]²⁺ calcd for C₇₈H₁₀₄FN₁₃O₇S₃: 724.8659. found: 724.8661.

### Example 80: 6-[3-(1,3-benzothiazol-2-ylamino)-4-methyl-6,7-dihydro-5H-pyrido[2,3-c]pyridazin-8-yl]-3-[1-[[3-[2-[14-[(2R)-2-[(1-fluorocyclopropanecarbonyl)amino]-3-[(2S,4R)-4-hydroxy-2-[[4-(4-methylthiazol-5-yl)phenyl]methylcarbamoyl]pyrrolidin-1-yl]-1,1-dimethyl-3-oxo-propyl]sulfanyltetradecyl-methyl-amino]ethoxy]-5,7-dimethyl-1-adamantyl]methyl]-5-methyl-pyrazol-4-yl]pyridine-2-carboxylic acid

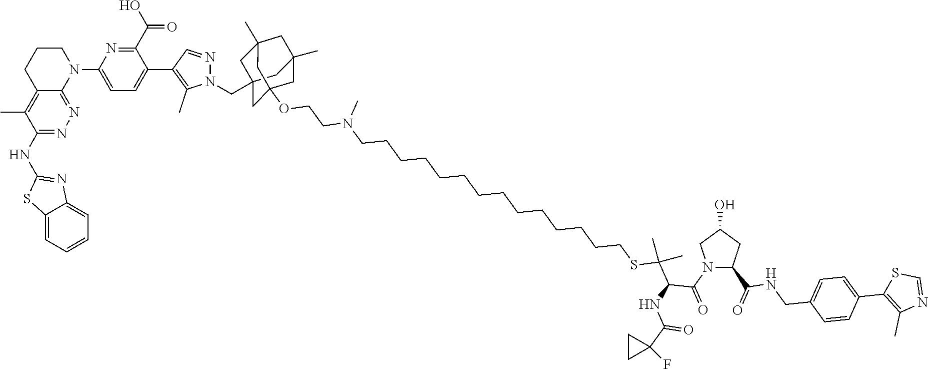 [SMILES: Cc1ncsc1-c1ccc(CNC(=O)[C@@H]2C[C@@H](O)CN2C(=O)[C@@H](NC(=O)C2(F)CC2)C(C)(C)SCCCCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)cc1]

### Step A: (2S,4R)-1-[(2R)-3-(14-bromotetradecylsulfanyl)-2-[(1-fluorocyclopropanecarbonyl) amino]-3-methyl-butanoyl]-4-hydroxy-N-[[4-(4-methylthiazol-5-yl)phenyl]methyl] pyrrolidine-2-carboxamide

Using General procedure for the alkylation of VHL ligand on thiol group starting from (2S,4R)-1-[(2R)-2-[(1-fluorocyclopropanecarbonyl)amino]-3-methyl-3-sulfanyl-butanoyl]-4-hydroxy-N-[[4-(4-methylthiazol-5-yl)phenyl]methyl]pyrrolidine-2-carboxamide (0.19 mmol) and 1,14-dibromotetradecane as the appropriate reactant, 67 mg of the desired product were obtained. ¹H NMR (500 MHz, DMSO-d₆): δ ppm 8.98 (s, 1H), 8.58 (t, 1H), 7.48 (dd, 1H), 7.42 (dm, 2H), 7.39 (dm, 2H), 5.19 (d, 1H), 4.78 (d, 1H), 4.46 (t, 1H), 4.43/4.23 (dd+dd, 2H), 4.36 (br., 1H), 3.72/3.64 (dd+dd, 2H), 3.51 (t, 2H), 2.51 (m, 2H), 2.45 (s, 3H), 2.08/1.91 (m+m, 2H), 1.77 (m, 2H), 1.42-1.13 (m, 22H), 1.38/1.35 (s+s, 6H), 1.38/1.21 (m+m, 4H); ¹³C NMR (125 MHz, DMSO-d₆) δ ppm 172.0, 168.6, 168.3, 151.9, 129.2, 127.9, 78.5, 69.3, 59.5, 57.0, 55.3, 49.5, 42.1, 38.4, 35.7, 32.7, 28.2, 27.2/24.7, 16.4, 13.4; HRMS-ESI (m/z): [M+H]⁺ calcd for C₃₉H₅₉BrFN₄O₄S₂: 809.3140. found: 809.3140.

### Step B: 6-[3-(1,3-benzothiazol-2-ylamino)-4-methyl-6,7-dihydro-5H-pyrido[2,3-c]pyridazin-8-yl]-3-[1-[[3-[2-[14-[(2R)-2-[(1-fluorocyclopropanecarbonyl)amino]-3-[(2S,4R)-4-hydroxy-2-[[4-(4-methylthiazol-5-yl)phenyl]methylcarbamoyl]pyrrolidin-1-yl]-1,1-dimethyl-3-oxo-propyl]sulfanyltetradecyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (40 mg) and the product of Step A as the appropriate alkylating agent, 20 mg of the desired product were obtained. HRMS-ESI (m/z): [M+2H]²⁺ calcd for C₈₀H₁₀₈FN₁₃O₇S₃: 738.8815. found: 738.8820.

### Example 81: 6-[3-(1,3-benzothiazol-2-ylamino)-4-methyl-6,7-dihydro-5H-pyrido[2,3-c]pyridazin-8-yl]-3-[1-[[3-[2-[[8-[4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]-1-piperidyl]-8-oxo-octyl]-methyl-amino]ethoxy]-5,7-dimethyl-1-adamantyl]methyl]-5-methyl-pyrazol-4-yl]pyridine-2-carboxylic acid

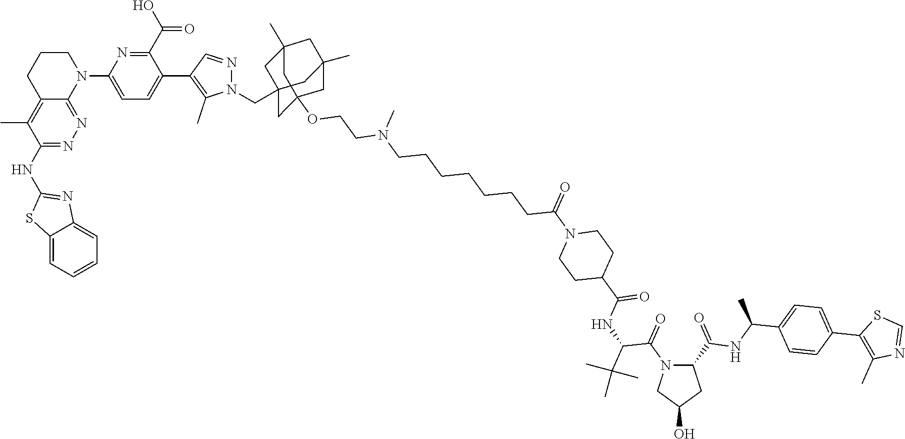 [SMILES: Cc1ncsc1-c1ccc([C@H](C)NC(=O)[C@@H]2C[C@@H](O)CN2C(=O)[C@@H](NC(=O)C2CCN(C(=O)CCCCCCCN(C)CCOC34CC5(C)CC(C)(CC(Cn6ncc(-c7ccc(N8CCCc9c8nnc(Nc8nc%10ccccc%10s8)c9C)nc7C(=O)O)c6C)(C5)C3)C4)CC2)C(C)(C)C)cc1]

### Step A: 1-(8-bromooctanoyl)-N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]piperidine-4-carboxamide

Using General procedure for the acylation of piperidinyl-VHL ligand starting from 8-bromooctanoic acid as the appropriate acid, 50 mg of the desired product were obtained. ¹H NMR (500 MHz, DMSO-d₆): δ ppm 8.99 (s, 1H), 8.40 (d, 1H), 7.84 (d, 1H), 7.44 (d, 2H), 7.38 (d, 2H), 5.12 (brs, 1H), 4.92 (qn, 1H), 4.50 (d, 1H), 4.43 (t, 1H), 4.37/3.87/2.97/2.50 (d+t/d+t, 4H), 4.28 (brm, 1H), 3.62/3.57 (dd+dd, 2H), 3.53 (t, 2H), 2.62 (t, 1H), 2.46 (s, 3H), 2.28 (t, 2H), 2.02/1.78 (n+n, 2H), 1.79 (qn, 2H), 1.74/1.62/1.47/1.42 (t+d/t+d, 4H), 1.47 (qn, 2H), 1.41-1.23 (m, 6H), 1.38 (d, 3H), 0.94 (s, 9H); ¹³C NMR (125 MHz, DMSO-d₆) δ ppm 151.9, 129.3, 126.8, 69.3, 59.0, 56.8, 56.7, 48.2, 45.0/41.1, 41.5, 38.2, 35.6, 32.7, 32.7, 30.3/29.1, 26.9, 25.3, 22.9, 16.5; HRMS-ESI (m/z): [M+H]⁺ calcd for C₃₇H₅₅BrN₅O₅S: 760.3102. found: 760.3103.

### Step B: 6-[3-(1,3-benzothiazol-2-ylamino)-4-methyl-6,7-dihydro-5H-pyrido[2,3-c]pyridazin-8-yl]-3-[1-[[3-[2-[[8-[4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]-1-piperidyl]-8-oxo-octyl]-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (33 mg) and the product of Step A as the appropriate alkylating agent, 18 mg of the desired product were obtained. HRMS-ESI (m/z): [M+2H]²⁺ calcd for C₇₈H₁₀₄N₁₄O₈S₂: 714.3796. found: 714.3797.

### Example 82: 6-[3-(1,3-benzothiazol-2-ylamino)-4-methyl-6,7-dihydro-5H-pyrido[2,3-c]pyridazin-8-yl]-3-[1-[[3-[2-[[11-[4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]-1-piperidyl]-11-oxo-undecyl]-methyl-amino]ethoxy]-5,7-dimethyl-1-adamantyl]methyl]-5-methyl-pyrazol-4-yl]pyridine-2-carboxylic acid

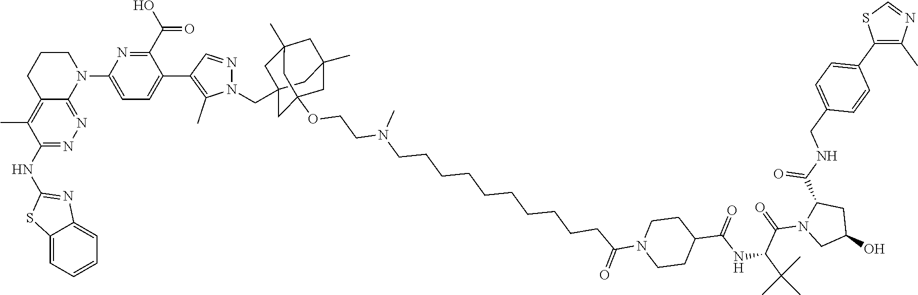

### Step A: 1-(11-bromoundecanoyl)-N-[(1S)-1-[(2S,4R)-4-hydroxy-2-[[(1S)-1-[4-(4-methyl thiazol-5-yl)phenyl]ethyl]carbamoyl]pyrrolidine-1-carbonyl]-2,2-dimethyl-propyl]piperidine-4-carboxamide

Using General procedure for the acylation of piperidinyl-VHL ligand starting from 11-bromoundecanoic acid as the appropriate acid, 46 mg of the desired product were obtained. HRMS-ESI (m/z): [M+H]⁺ calcd for C₄₀H₆₁BrN₅O₅S: 802.3571. found: 802.3569.

### Step B: 6-[3-(1,3-benzothiazol-2-ylamino)-4-methyl-6,7-dihydro-5H-pyrido[2,3-c]pyridazin-8-yl]-3-[1-[[3-[2-[[11-[4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]-1-piperidyl]-11-oxo-undecyl]-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (25 mg) and the product of Step A as the appropriate alkylating agent, 15 mg of the desired product were obtained. HRMS-ESI (m/z): [M+2H]²⁺ calcd for C₈₁H₁₁₀N₁₄O₈S₂: 735.4031. found: 735.4031.

### Example 83: 6-[3-(1,3-benzothiazol-2-ylamino)-4-methyl-6,7-dihydro-5H-pyrido[2,3-c]pyridazin-8-yl]-3-[1-[[3-[2-[15-[2-[[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]pentadecyl-methyl-amino]ethoxy]-5,7-dimethyl-1-adamantyl]methyl]-5-methyl-pyrazol-4-yl]pyridine-2-carboxylic acid

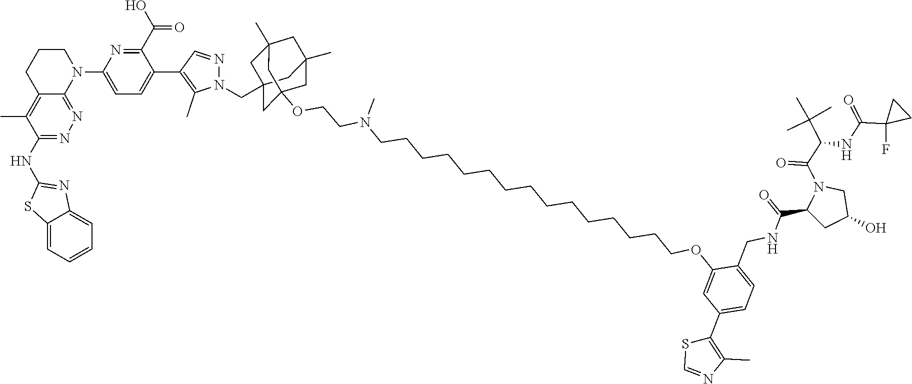 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)c1]

### Step A: (2S,4R)—N-[[2-(15-bromopentadecoxy)-4-(4-methylthiazol-5-yl)phenyl]methyl]-1-[(2S)-2-[(1-fluorocyclopropanecarbonyl)amino]-3,3-dimethyl-butanoyl]-4-hydroxy-pyrrolidine-2-carboxamide

Using General procedure for the alkylation of VHL ligand on hydroxy group starting from (2S,4R)-1-[(2S)-2-[(1-fluorocyclopropanecarbonyl)amino]-3,3-dimethyl-butanoyl]-4-hydroxy-N-[[2-hydroxy-4-(4-methylthiazol-5-yl)phenyl]methyl]pyrrolidine-2-carboxamide (0.38 mmol) and 1,15-dibromopentadecane as the appropriate reactant, 34 mg of the desired product were obtained. HRMS-ESI (m/z): [M+H]⁺ calcd for C₄₁H₆₃BrFN₄O₅S: 821.3681. found: 821.3678.

### Step B: 6-[3-(1,3-benzothiazol-2-ylamino)-4-methyl-6,7-dihydro-5H-pyrido[2,3-c]pyridazin-8-yl]-3-[1-[[3-[2-[15-[2-[[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]pentadecyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (20 mg) and the product of Step A as the appropriate alkylating agent, 7 mg of the desired product were obtained. HRMS-ESI (m/z): [M+2H]²⁺ calcd for C₂H₁₁₂FN₁₃O₈S₂: 744.9086. found: 744.9089.

### Example 84: 6-[3-(1,3-benzothiazol-2-ylamino)-4-methyl-6,7-dihydro-5H-pyrido[2,3-c]pyridazin-8-yl]-3-[1-[[3-[2-[[10-[4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]-1-piperidyl]-10-oxo-decyl]-methyl-amino]ethoxy]-5,7-dimethyl-1-adamantyl]methyl]-5-methyl-pyrazol-4-yl]pyridine-2-carboxylic acid

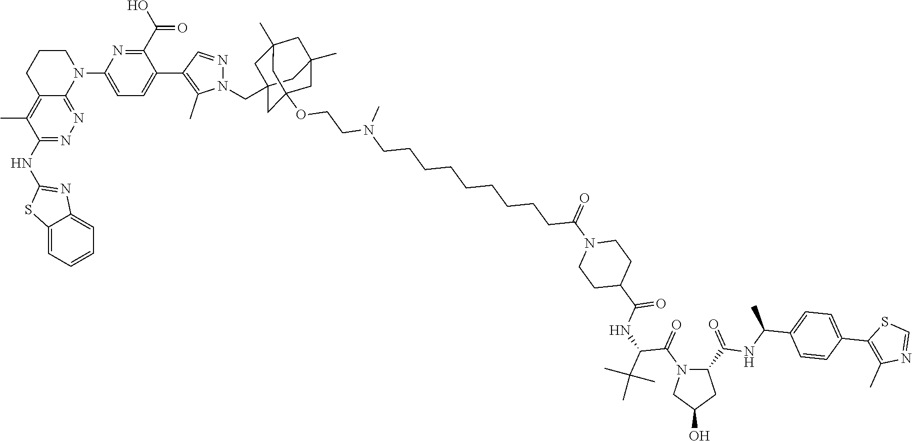 [SMILES: Cc1ncsc1-c1ccc([C@H](C)NC(=O)[C@@H]2C[C@@H](O)CN2C(=O)[C@@H](NC(=O)C2CCN(C(=O)CCCCCCCCCN(C)CCOC34CC5(C)CC(C)(CC(Cn6ncc(-c7ccc(N8CCCc9c8nnc(Nc8nc%10ccccc%10s8)c9C)nc7C(=O)O)c6C)(C5)C3)C4)CC2)C(C)(C)C)cc1]

### Step A: 1-(10-bromodecanoyl)-N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]piperidine-4-carboxamide

Using General procedure for the acylation of piperidinyl-VHL ligand starting from 10-bromodecanoic acid as the appropriate acid, 81 mg of the desired product were obtained. HRMS-ESI (m/z): [M+H]⁺ calcd for C₃₉H₅₉BrN₅O₅S: 788.3415. found: 788.3415.

### Step B: 6-[3-(1,3-benzothiazol-2-ylamino)-4-methyl-6,7-dihydro-5H-pyrido[2,3-c]pyridazin-8-yl]-3-[1-[[3-[2-[[10-[4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]-1-piperidyl]-10-oxo-decyl]-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (40 mg) and the product of Step A as the appropriate alkylating agent, 15 mg of the desired product were obtained. HRMS-ESI (m/z): [M+2H]²⁺ calcd for C₈₀H₁₀₈N₁₄O₈S₂: 728.3953. found: 728.3953.

### Example 85: 6-[3-(1,3-benzothiazol-2-ylamino)-4-methyl-6,7-dihydro-5H-pyrido[2,3-c]pyridazin-8-yl]-3-[1-[[3-[2-[13-[2-[[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]tridecyl-methyl-amino]ethoxy]-5,7-dimethyl-1-adamantyl]methyl]-5-methyl-pyrazol-4-yl]pyridine-2-carboxylic acid

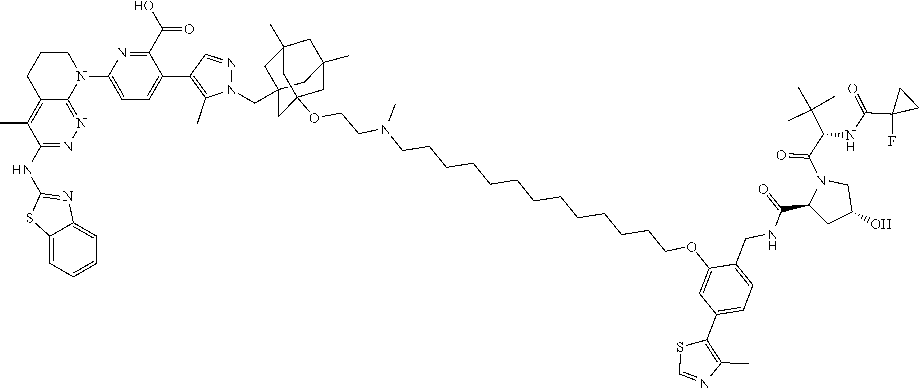 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)c1]

### Step A: (2S,4R)—N-[[2-(13-bromotridecoxy)-4-(4-methylthiazol-5-yl)phenyl]methyl]-1-[(2S)-2-[(1-fluorocyclopropanecarbonyl)amino]-3,3-dimethyl-butanoyl]-4-hydroxy-pyrrolidine-2-carboxamide

Using General procedure for the alkylation of VHL ligand on hydroxy group starting from (2S,4R)-1-[(2S)-2-[(1-fluorocyclopropanecarbonyl)amino]-3,3-dimethyl-butanoyl]-4-hydroxy-N-[[2-hydroxy-4-(4-methylthiazol-5-yl)phenyl]methyl]pyrrolidine-2-carboxamide (0.38 mmol) and 1,13-dibromotridecane as the appropriate reactant, 109 mg of the desired product were obtained. LC-MS-ESI (m/z): [M+H]⁺ calcd for C₃₉H₅₉BrFN₄O₅S: 795. found: 795.

### Step B: 6-[3-(1,3-benzothiazol-2-ylamino)-4-methyl-6,7-dihydro-5H-pyrido[2,3-c]pyridazin-8-yl]-3-[1-[[3-[2-[13-[2-[[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]tridecyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (40 mg) and the product of Step A as the appropriate alkylating agent, 35 mg of the desired product were obtained. HRMS-ESI (m/z): [M+2H]²⁺ calcd for C₈₀H₁₀₈FN₁₃O₈S₂: 730.8929. found: 730.8931.

### Example 86: 6-[3-(1,3-benzothiazol-2-ylamino)-4-methyl-6,7-dihydro-5H-pyrido[2,3-c]pyridazin-8-yl]-3-[1-[[3-[2-[[12-[4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]-1-piperidyl]-12-oxo-dodecyl]-methyl-amino]ethoxy]-5,7-dimethyl-1-adamantyl]methyl]-5-methyl-pyrazol-4-yl]pyridine-2-carboxylic acid

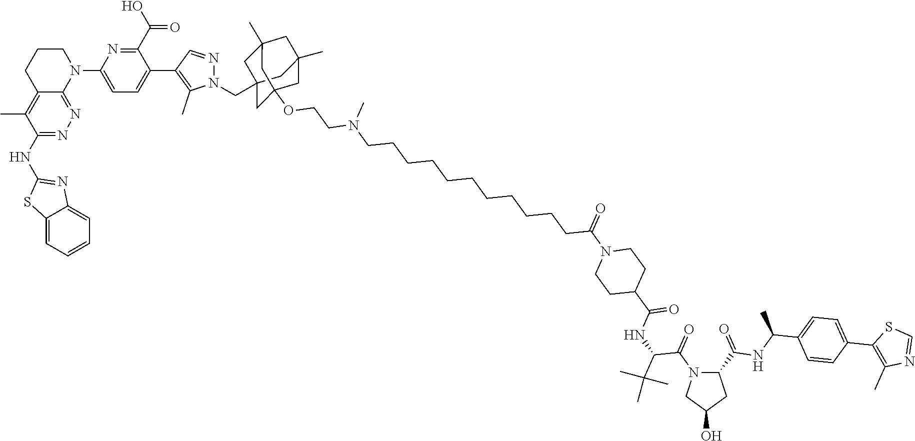 [SMILES: Cc1ncsc1-c1ccc([C@H](C)NC(=O)[C@@H]2C[C@@H](O)CN2C(=O)[C@@H](NC(=O)C2CCN(C(=O)CCCCCCCCCCCN(C)CCOC34CC5(C)CC(C)(CC(Cn6ncc(-c7ccc(N8CCCc9c8nnc(Nc8nc%10ccccc%10s8)c9C)nc7C(=O)O)c6C)(C5)C3)C4)CC2)C(C)(C)C)cc1]

### Step A: 1-(12-bromododecanoyl)-N-[(1S)-1-[(2S,4R)-4-hydroxy-2-[[(1S)-1-[4-(4-methyl thiazol-5-yl)phenyl]ethyl]carbamoyl]pyrrolidine-1-carbonyl]-2,2-dimethyl-propyl]piperidine-4-carboxamide

Using General procedure for the acylation of piperidinyl-VHL ligand starting from 12-bromododecanoic acid as the appropriate acid, 49 mg of the desired product were obtained. HRMS-ESI (m/z): [M+H]⁺ calcd for C₄₁H₆₃BrN₅O₅S: 816.3728. found: 816.3728.

### Step B: 6-[3-(1,3-benzothiazol-2-ylamino)-4-methyl-6,7-dihydro-5H-pyrido[2,3-c]pyridazin-8-yl]-3-[1-[[3-[2-[[12-[4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]-1-piperidyl]-12-oxo-dodecyl]-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (32 mg) and the product of Step A as the appropriate alkylating agent, 12 mg of the desired product were obtained. HRMS-ESI (m/z): [M+2H]²⁺ calcd for C₈₂H₁₁₂N₁₄O₈S₂: 742.4109. found: 742.4110.

### Example 87: 6-[3-(1,3-benzothiazol-2-ylamino)-4-methyl-6,7-dihydro-5H-pyrido[2,3-c]pyridazin-8-yl]-3-[1-[[3-[2-[2-[2-[2-[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]ethoxy]ethoxy]ethyl-methyl-amino]ethoxy]-5,7-dimethyl-1-adamantyl]methyl]-5-methyl-pyrazol-4-yl]pyridine-2-carboxylic acid

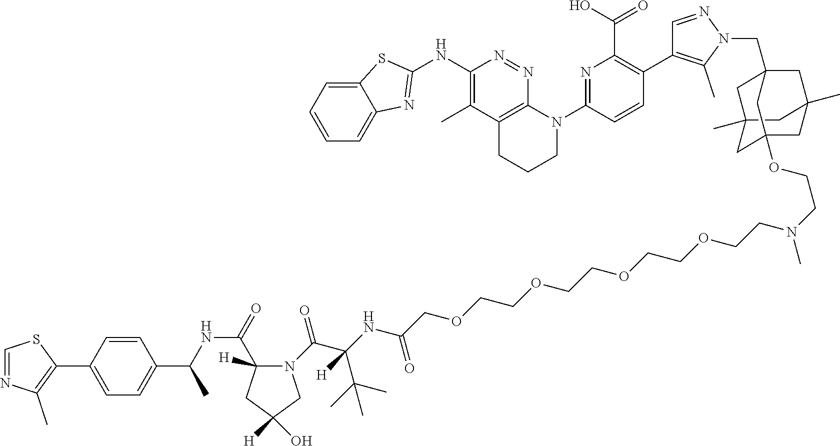 [SMILES: Cc1ncsc1-c1ccc([C@H](C)NC(=O)[C@H]2C[C@@H](O)CN2C(=O)[C@H](NC(=O)COCCOCCOCCOCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1]

### Step A: tert-butyl 2-[2-[2-[2-[2-(p-tolylsulfonyloxy)ethoxy]ethoxy]ethoxy]ethoxy]acetate

Using General procedure for the tosylation of the hydroxyalkyl VHL ligand-derivatives starting from 1.5 g (4.86 mmol) of tert-butyl 2-[2-[2-[2-(2-hydroxyethoxy)ethoxy]ethoxy]ethoxy]acetate, 1.6 g of the desired product was obtained. HPLC-MS-ESI (TFA) m/z=462.

### Step B: 2-[2-[2-[2-[2-(p-tolylsulfonyloxy)ethoxy]ethoxy]ethoxy]ethoxy]acetic acid

A mixture of 1.60 g of the product of Step A, DCM (6.0 mL) and TFA (2.4 mL, 9 eq) was stirred at RT for 18 h. 1.25 g of the desired product was obtained after removal the volatiles. ¹H NMR (400 MHz, DMSO-d₆) δ ppm 12.5 (brs, 1H), 7.79 (m, 2H), 7.48 (m, 2H), 4.11 (m, 2H), 4.01 (s, 2H), 3.61-3.42 (m, 12H), 3.57 (m, 2H), 2.42 (s, 3H); ¹³C NMR (100 MHz, DMSO-d₆) δ ppm 172.1, 145.4, 132.8, 130.6, 128.1, 70.5, 68.3, 68.0, 21.6; HRMS-ESI (m/z): [M+H]⁺ calcd for C₁₇H₂₆O₉S: 407.1370. found 407.1369.

### Step C: 2-[2-[2-[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]ethoxy]ethoxy]ethyl 4-methylbenzenesulfonate

Using General procedure for the acylation of VHL ligands starting from 300 mg of (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.62 mmol) and the product of Step B as the appropriate acid, 439 mg of the desired product were obtained. ¹H NMR (400 MHz, DMSO-d₆) δ ppm 8.99 (s, 1H), 8.45 (d, 1H), 7.79 (d, 2H), 7.49 (d, 2H), 7.45 (d, 2H), 7.39 (d, 1H), 7.37 (d, 2H), 4.90 (qn, 1H), 4.55 (d, 1H), 4.45 (t, 1H), 4.29 (brm, 1H), 4.12 (t, 2H), 3.96 (s, 2H), 3.64-3.48 (m, 14H), 3.60/3.57 (dd+dd, 2H), 2.46 (s, 3H), 2.43 (s, 3H), 2.05/1.77 (m+m, 2H), 1.37 (d, 3H), 0.94 (s, 9H); ¹³C NMR (100 MHz, DMSO-d₆) δ ppm 151.9, 130.6, 129.3, 128.1, 126.8, 70.5, 70.0, 69.3, 59.0, 57.0, 56.1, 48.2, 38.2, 26.7, 23.0, 21.6, 16.5; HRMS-ESI (m/z): [M+H]⁺ calcd for C₄₀H₅₆N₄O₁₁S₂: 833.3460. found: 833.3461.

### Step D: 6-[3-(1,3-benzothiazol-2-ylamino)-4-methyl-6,7-dihydro-5H-pyrido[2,3-c]pyridazin-8-yl]-3-[1-[[3-[2-[2-[2-[2-[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]ethoxy]ethoxy]ethyl-methyl-amino]ethoxy]-5,7-dimethyl-1-adamantyl]methyl]-5-methyl-pyrazol-4-yl]pyridine-2-carboxylic acid

Using Degrader Synthesis by Alkylation and Hydrolysis General Procedure starting from the product of Step C as the appropriate alkylating agent and 30 mg (0.03 mmol) Preparation 4 as the appropriate amine, 28 mg of the desired product were obtained. HRMS-ESI (m/z): [M+2H]²⁺ calcd for C₇₄H₉₉N₁₃O₁₁S₂: 704.8509. found: 704.8510.

### Example 88: 6-[3-(1,3-benzothiazol-2-ylamino)-4-methyl-6,7-dihydro-5H-pyrido[2,3-c]pyridazin-8-yl]-3-[1-[[3-[2-[2-[2-[2-[3-[[(1S)-1-[(2R,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]ethoxy]ethoxy]ethyl-methyl-amino]ethoxy]-5,7-dimethyl-1-adamantyl]methyl]-5-methyl-pyrazol-4-yl]pyridine-2-carboxylic acid

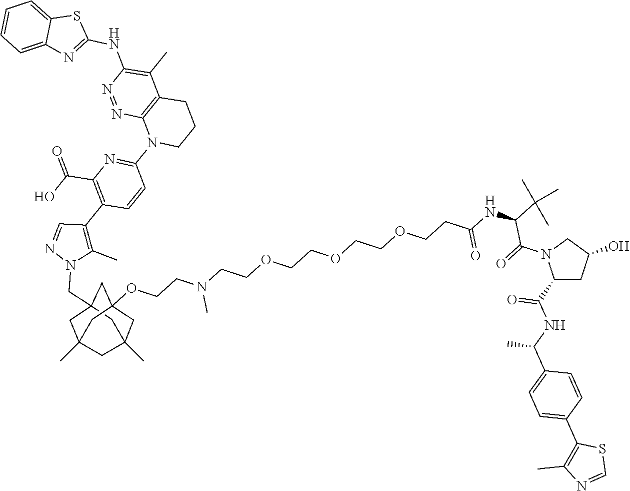 [SMILES: Cc1ncsc1-c1ccc([C@H](C)NC(=O)[C@H]2C[C@@H](O)CN2C(=O)[C@@H](NC(=O)CCOCCOCCOCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1]

### Step A: (2S,4R)-1-[(2S)-2-[3-[2-[2-(2-bromoethoxy)ethoxy]ethoxy]propanoylamino]-3,3-dimethyl-butanoyl]-4-hydroxy-N-[(1S)-1-[4-(4-methylthiazol-5-yl)phenyl]ethyl]pyrrolidine-2-carboxamide

Using General procedure for the acylation of VHL ligands starting from 300 mg (0.62 mmol) of (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) and 3-[2-[2-(2-bromoethoxy)ethoxy]ethoxy]propanoic acid as the appropriate acid, 400 mg of the desired product were obtained. ¹H NMR (400 MHz, DMSO-d₆) δ ppm 8.98 (s, 1H), 8.39 (d, 1H), 7.87 (d, 1H), 7.44 (m, 2H), 7.38 (m, 2H), 5.11 (d, 1H), 4.91 (m, 1H), 4.52 (d, 1H), 4.42 (t, 1H), 4.28 (m, 1H), 3.73 (t, 2H), 3.65-3.44 (m, 10H), 3.64-3.53 (m, 2H), 3.58 (t, 2H), 2.53/2.35 (m+m, 2H), 2.45 (s, 3H), 2.01/1.78 (m+m, 2H), 1.37 (d, 3H), 0.93 (s, 9H); ¹³C NMR (100 MHz, DMSO-d₆) δ ppm 152.0, 129.3, 126.8, 70.8, 69.2, 59.0, 56.8, 56.8, 48.2, 38.2, 36.1, 32.8, 26.9, 22.9, 16.5; HRMS-ESI (m/z): [M+H]⁺ calcd for for C₃₂H₄₈BrN₄O₇S: 711.2422. found: 711.2434.

### Step B: 6-[3-(1,3-benzothiazol-2-ylamino)-4-methyl-6,7-dihydro-5H-pyrido[2,3-c]pyridazin-8-yl]-3-[1-[[3-[2-[2-[2-[2-[3-[[(1S)-1-[(2R,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]ethoxy]ethoxy]ethyl-methyl-amino]ethoxy]-5,7-dimethyl-1-adamantyl]methyl]-5-methyl-pyrazol-4-yl]pyridine-2-carboxylic acid

Using Degrader Synthesis by Alkylation and Hydrolysis General Procedure starting from the product of Step A as the appropriate alkylating agent and 50 mg (0.03 mmol) Preparation 4 as the appropriate amine, 72 mg of the desired product were obtained. HRMS-ESI (m/z): [M+2H]²⁺ calcd for C₇₄H₉₇N₁₃O₁₁S₂: 689.8456. found: 689.8457.

### Example 89: 6-[3-(1,3-benzothiazol-2-ylamino)-4-methyl-6,7-dihydro-5H-pyrido[2,3-c]pyridazin-8-yl]-3-[1-[[3-[2-[2-[2-[2-[2-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]ethoxy]ethoxy]ethoxy]ethyl-methyl-amino]ethoxy]-5,7-dimethyl-1-adamantyl]methyl]-5-methyl-pyrazol-4-yl]pyridine-2-carboxylic acid

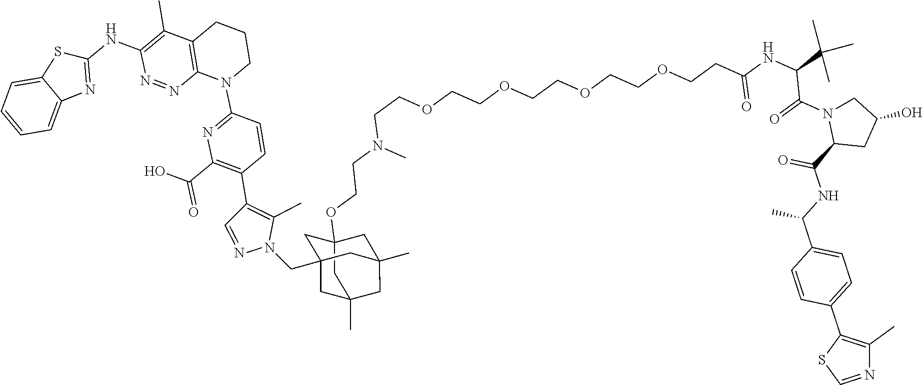 [SMILES: Cc1ncsc1-c1ccc([C@H](C)NC(=O)[C@@H]2C[C@@H](O)CN2C(=O)[C@@H](NC(=O)CCOCCOCCOCCOCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1]

### Step A: tert-butyl 3-methoxypropanoate;2-[2-(2-ethoxyethoxy)ethoxy]ethyl 4-methyl benzenesulfonate

Using General procedure for the tosylation of the hydroxyalkyl VHL ligand-derivatives starting from 1.5 g (4.65 mmol) of tert-butyl 3-[2-[2-[2-(2-hydroxyethoxy)ethoxy]ethoxy]ethoxy]propanoate, 0.9 g of the product was obtained. HPLC-MS-ESI (TFA) m/z=427.

### Step B: 2-[2-[2-[2-[2-(p-tolylsulfonyloxy)ethoxy]ethoxy]ethoxy]ethoxy]acetic acid

A mixture of 1.30 g of the product of Step A, DCM (6.0 mL) and TFA (1.8 mL, 9 eq) was stirred at RT for 18 h. 1.00 g of the desired product was obtained after removal the volatiles. ¹H NMR (400 MHz, DMSO-d₆) δ ppm 12.00 (brs, 1H), 7.79 (m, 2H), 7.49 (m, 2H), 4.11 (m, 2H), 3.59 (t, 2H), 3.57 (m, 2H), 3.53-3.40 (m, 12H), 2.44 (t, 2H), 2.43 (s, 3H); ¹³C NMR (100 MHz, DMSO-d₆) δ ppm 130.6, 128.2, 70.5, 68.3, 66.7, 35.2, 21.7; HRMS-ESI (m/z): [M+NH₄]⁺ calcd for C₁₈H₃₂NO₉S: 438.1792. found 438.1794.

### Step C: 2-[2-[2-[2-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]ethoxy]ethoxy]ethoxy]ethyl 4-methylbenzenesulfonate

Using General procedure for the acylation of VHL ligands from 300 mg (0.62 mmol) of (2S,4R)-1-[(2S)-2-amino-3,3-dimethyl-butanoyl]-4-hydroxy-N-[(1S)-1-[4-(4-methylthiazol-5-yl)phenyl] ethyl] pyrrolidine-2-carboxamide, hydrogen chloride (1:1) and the product of Step B as the appropriate acid, 518 mg of the desired product were obtained. ¹H NMR (400 MHz, DMSO-d₆) δ ppm 8.99 (s, 1H), 8.40 (d, 1H), 7.88 (d, 1H), 7.79 (d, 2H), 7.49 (d, 2H), 7.44 (d, 2H), 7.38 (d, 2H), 5.13 (d, 1H), 4.92 (qn, 1H), 4.54 (d, 1H), 4.43 (t, 1H), 4.28 (brm, 1H), 4.12 (t, 2H), 3.60/3.57 (dd+dd, 2H), 3.60 (t, 2H), 3.57 (t, 2H), 3.52-3.44 (m, 12H), 2.52/2.33 (m+m, 2H), 2.46 (s, 3H), 2.43 (s, 3H), 2.02/1.78 (m+m, 2H), 1.38 (d, 3H), 0.94 (s, 9H); ¹³C NMR (100 MHz, DMSO-d₆) δ ppm 152.0, 130.6, 129.3, 128.1, 126.8, 70.5, 69.2, 68.4, 67.4, 59.0, 56.8, 56.8, 48.2, 38.2, 36.1, 26.9, 22.9, 21.6, 16.5; HRMS-ESI (m/z): [M+H]⁺ calcd for C₄₁H₅₉N₄O₁₁S₂: 847.3616. found: 847.3612.

### Step D: 6-[3-(1,3-benzothiazol-2-ylamino)-4-methyl-6,7-dihydro-5H-pyrido[2,3-c]pyridazin-8-yl]-3-[1-[[3-[2-[2-[2-[2-[2-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]ethoxy]ethoxy]ethoxy]ethyl-methyl-amino]ethoxy]-5,7-dimethyl-1-adamantyl]methyl]-5-methyl-pyrazol-4-yl]pyridine-2-carboxylic acid

Using Degrader Synthesis by Alkylation and Hydrolysis General Procedure starting from the product of Step C as the appropriate alkylating agent and 70 mg (0.03 mmol) Preparation 4 as the appropriate amine, 44 mg of the desired product were obtained. HRMS-ESI (m/z): [M+2H]²⁺ calcd for C₇₅H₁₀₁N₁₃O₁₁S₂: 711.8587. found: 711.8587.

### Example 90: 6-[[6-(1,3-benzothiazol-2-ylamino)-5-methyl-pyridazin-3-yl]-[4-[11-[[2-(2,6-dioxo-3-piperidyl)-1,3-dioxo-isoindolin-4-yl]amino]undecanoyl-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

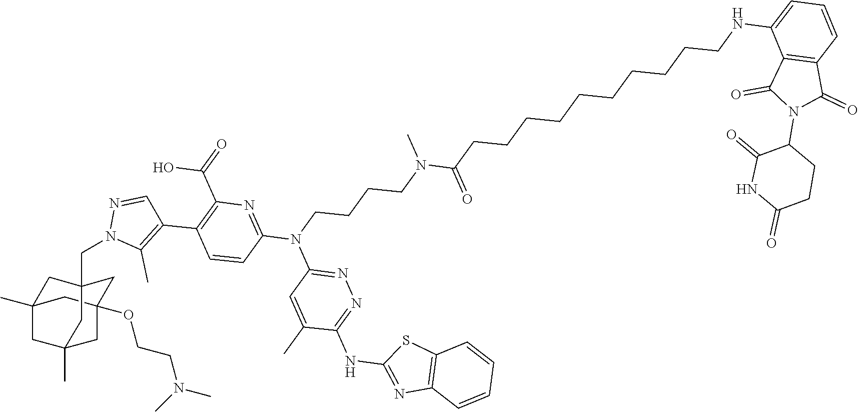 [SMILES: Cc1cc(N(CCCCN(C)C(=O)CCCCCCCCCCNc2cccc3c2C(=O)N(C2CCC(=O)NC2=O)C3=O)c2ccc(-c3cnn(CC45CC6(C)CC(C)(C4)CC(OCCN(C)C)(C6)C5)c3C)c(C(=O)O)n2)nnc1Nc1nc2ccccc2s1]

### Step A: 11-[[2-(2,6-dioxo-3-piperidyl)-1,3-dioxo-isoindolin-4-yl]amino]undecanoic acid

Using General procedure for the nucleophilic substitution of fluoro-thalidomide starting from 276 mg (1.00 mmol) of 2-(2,6-dioxo-3-piperidyl)-4-fluoro-isoindoline-1,3-dione and 11-aminoundecanoic acid as the appropriate amine, 72 mg of the desired product were obtained. ¹H NMR (400 MHz, DMSO-d₆) δ ppm 11.97 (brs, 1H), 11.10 (s, 1H), 7.58 (dd, 1H), 7.09 (d, 1H), 7.01 (d, 1H), 6.53 (t, 1H), 5.05 (dd, 1H), 3.28 (m, 2H), 2.88/2.59 (m+m, 2H), 2.54/2.03 (m+m, 2H), 2.17 (t, 2H), 1.58 (m, 2H), 1.47 (m, 2H), 1.40-1.18 (m, 12H); ¹³C NMR (100 MHz, DMSO-d₆) δ ppm 136.7, 117.7, 110.8, 49.0, 42.3, 34.1, 31.5, 29.1, 25.0, 22.6; HRMS-ESI (m/z): [M+H]⁺ calcd for C₂₄H₃₂N₃O₆: 458.2286. found: 458.2293.

### Step B: 6-[[6-(1,3-benzothiazol-2-ylamino)-5-methyl-pyridazin-3-yl]-[4-[11-[[2-(2,6-dioxo-3-piperidyl)-1,3-dioxo-isoindolin-4-yl]amino]undecanoyl-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

Using Degrader Synthesis by Amide Coupling and Hydrolysis General Procedure starting from the product of Step A as the appropriate acid and 40 mg (0.04 mmol) of Preparation 19 as the appropriate amine, 20 mg of the desired product were obtained. HRMS-ESI (m/z): [M+H]⁺ calcd for C₆₈H₈₈N₁₃O₈S: 1246.6594. found 1246.6590.

### Example 91: 6-[[6-(1,3-benzothiazol-2-ylamino)-5-methyl-pyridazin-3-yl]-[4-[9-[[2-(2,6-dioxo-3-piperidyl)-1,3-dioxo-isoindolin-4-yl]amino]nonanoyl-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

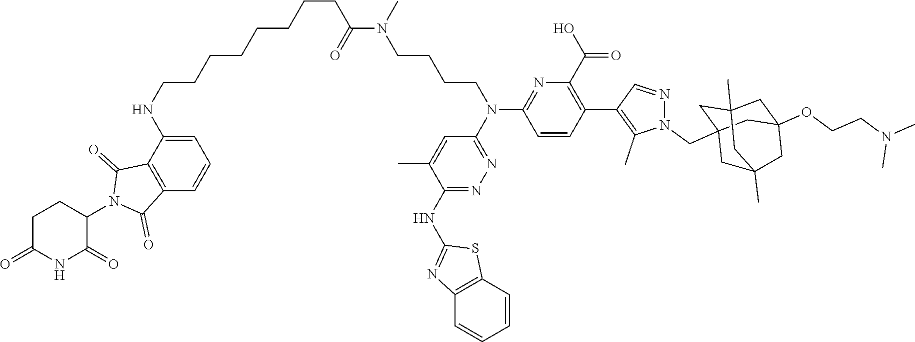 [SMILES: Cc1cc(N(CCCCN(C)C(=O)CCCCCCCCNc2cccc3c2C(=O)N(C2CCC(=O)NC2=O)C3=O)c2ccc(-c3cnn(CC45CC6(C)CC(C)(C4)CC(OCCN(C)C)(C6)C5)c3C)c(C(=O)O)n2)nnc1Nc1nc2ccccc2s1]

### Step A: 9-[[2-(2,6-dioxo-3-piperidyl)-1,3-dioxo-isoindolin-4-yl]amino]nonanoic acid

Using General procedure for the nucleophilic substitution of fluoro-thalidomide starting from 276 mg (1.00 mmol) of 2-(2,6-dioxo-3-piperidyl)-4-fluoro-isoindoline-1,3-dione and 9-aminononanoic acid as the appropriate amine, 98 mg of the desired product were obtained. ¹H NMR (500 MHz, DMSO-d₆) δ ppm 11.96 (s, 1H), 11.10 (s, 1H), 7.58 (t, 1H), 7.09 (dd, 1H), 7.02 (d, 1H), 6.53 (t, 1H), 5.05 (dd, 1H), 3.29 (q, 2H), 2.88/2.59 (td+dd, 2H), 2.52/2.02 (dd+dq, 2H), 2.18 (t, 2H), 1.57 (qn, 2H), 1.48 (qn, 2H), 1.35-1.24 (m, 6H), 1.33 (qn, 2H); ¹³C NMR (125 MHz, DMSO-d₆) δ ppm 175.0, 173.3, 170.6, 169.4, 167.8, 146.9, 136.8, 132.6, 117.7, 110.8, 109.5, 49.0, 42.3, 34.1, 31.4, 29.1, 26.8, 24.9, 22.6; HRMS-ESI (m/z): [M+H]⁺ calcd for C₂₂H₂₈NO: 430.1973. found: 430.1975.

### Step B: 6-[[6-(1,3-benzothiazol-2-ylamino)-5-methyl-pyridazin-3-yl]-[4-[9-[[2-(2,6-dioxo-3-piperidyl)-1,3-dioxo-isoindolin-4-yl]amino]nonanoyl-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

Using Degrader Synthesis by Amide Coupling General Procedure and Hydrolysis General Procedure starting from the product of Step A as the appropriate acid and 40 mg (0.04 mmol) of Preparation 19 as the appropriate amine, 31 mg of the desired product were obtained. HRMS-ESI (m/z): [M+H]⁺ calcd for C₆₆H₈₄N₁₃O₈S: 1218.6281. found 1218.6286.

### Example 92: 6-[[6-(1,3-benzothiazol-2-ylamino)-5-methyl-pyridazin-3-yl]-[4-[10-[[2-(2,6-dioxo-3-piperidyl)-1,3-dioxo-isoindolin-4-yl]amino]decanoyl-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

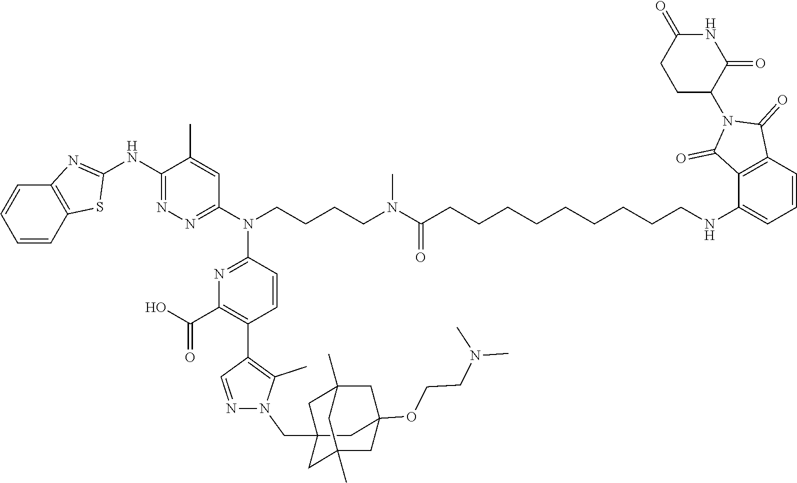 [SMILES: Cc1cc(N(CCCCN(C)C(=O)CCCCCCCCCNc2cccc3c2C(=O)N(C2CCC(=O)NC2=O)C3=O)c2ccc(-c3cnn(CC45CC6(C)CC(C)(C4)CC(OCCN(C)C)(C6)C5)c3C)c(C(=O)O)n2)nnc1Nc1nc2ccccc2s1]

### Step A: 10-[[2-(2,6-dioxo-3-piperidyl)-1,3-dioxo-isoindolin-4-yl]amino]decanoic acid

Using General procedure for the nucleophilic substitution of fluoro-thalidomide starting from 276 mg (1.00 mmol) of 2-(2,6-dioxo-3-piperidyl)-4-fluoro-isoindoline-1,3-dione and 10-aminodecanoic acid as the appropriate amine, 76 mg of the desired product were obtained. ¹H NMR (500 MHz, DMSO-d₆) δ ppm 11.96 (s, 1H), 11.09 (s, 1H), 7.58 (dd, 1H), 7.09 (d, 1H), 7.01 (d, 1H), 6.53 (t, 1H), 5.05 (dd, 1H), 3.29 (m, 2H), 2.88/2.58 (m+m, 2H), 2.51/2.02 (m+m, 2H), 2.18 (t, 2H), 1.57 (m, 2H), 1.47 (m, 2H), 1.39-1.19 (m, 10H); ¹³C NMR (125 MHz, DMSO-d₆) δ ppm 136.8, 117.7, 110.8, 49.0, 42.3, 34.1, 31.4, 29.1, 25.0, 22.6; HRMS-ESI (m/z): [M+H]⁺ calcd for C₂₃H₃₀N₃O₆: 444.2129. found: 444.2132.

### Step B: 6-[[6-(1,3-benzothiazol-2-ylamino)-5-methyl-pyridazin-3-yl]-[4-[10-[[2-(2,6-dioxo-3-piperidyl)-1,3-dioxo-isoindolin-4-yl]amino]decanoyl-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

Using Degrader Synthesis by Amide Coupling and Hydrolysis General Procedure starting from the product of Step A as the appropriate acid and 40 mg (0.04 mmol) of Preparation 19 as the appropriate amine, 49 mg of the desired product were obtained. HRMS-ESI (m/z): [M+H]⁺ calcd for C₆₇H₈₆N₁₃O₈S: 1232.6438. found 1232.6442.

### Example 93: 6-[[6-(1,3-benzothiazol-2-ylamino)-5-methyl-pyridazin-3-yl]-[4-[12-[[2-(2,6-dioxo-3-piperidyl)-1,3-dioxo-isoindolin-4-yl]amino]dodecanoyl-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

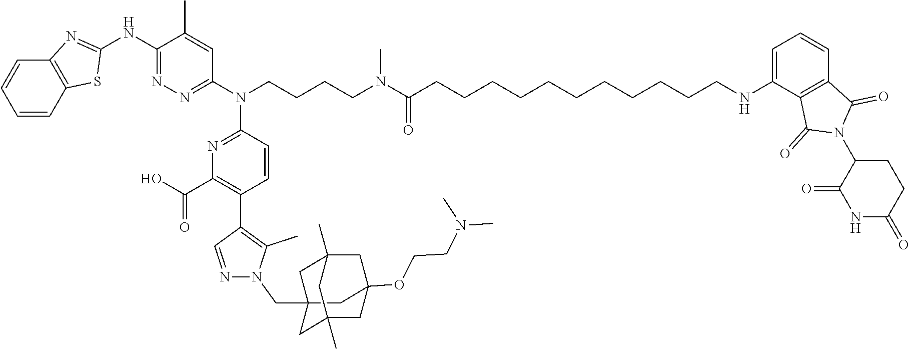 [SMILES: Cc1cc(N(CCCCN(C)C(=O)CCCCCCCCCCCNc2cccc3c2C(=O)N(C2CCC(=O)NC2=O)C3=O)c2ccc(-c3cnn(CC45CC6(C)CC(C)(C4)CC(OCCN(C)C)(C6)C5)c3C)c(C(=O)O)n2)nnc1Nc1nc2ccccc2s1]

### Step A: 12-[[2-(2,6-dioxo-3-piperidyl)-1,3-dioxo-isoindolin-4-yl]amino]dodecanoic acid

Using General procedure for the nucleophilic substitution of fluoro-thalidomide starting from 276 mg (1.00 mmol) of 2-(2,6-dioxo-3-piperidyl)-4-fluoro-isoindoline-1,3-dione and 12-aminododecanoic acid as the appropriate amine, 87 mg of the desired product were obtained. ¹H NMR (500 MHz, DMSO-d₆) δ ppm 11.96 (brs, 1H), 11.09 (s, 1H), 7.57 (dd, 1H), 7.08 (d, 1H), 7.01 (d, 1H), 6.52 (t, 1H), 5.05 (dd, 1H), 3.28 (q, 2H), 2.88/2.59 (td+dd, 2H), 2.51/2.02 (dd+dq, 2H), 2.17 (t, 2H), 1.56 (qn, 2H), 1.47 (qn, 2H), 1.32-1.22 (m, 12H), 1.32 (qn, 2H); ¹³C NMR (125 MHz, DMSO-d₆) δ ppm 175.0, 173.3, 170.6, 169.4, 167.8, 146.9, 136.8, 132.7, 117.7, 110.8, 109.4, 49.0, 42.3, 34.1, 31.5, 29.1, 26.8, 24.9, 22.6; HRMS-ESI (m/z): [M+H]⁺ calcd for C₂₅H₃₄N₃O₆: 472.2442. found: 472.2444.

### Step B: 6-[[6-(1,3-benzothiazol-2-ylamino)-5-methyl-pyridazin-3-yl]-[4-[12-[[2-(2,6-dioxo-3-piperidyl)-1,3-dioxo-isoindolin-4-yl]amino]dodecanoyl-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

Using Degrader Synthesis by Amide Coupling and Hydrolysis General Procedure starting from the product of Step A as the appropriate acid and 40 mg (0.04 mmol) of Preparation 19 as the appropriate amine, 20 mg of the desired product were obtained. HRMS-ESI (m/z): [M+H]⁺ calcd for C₆₉H₉₀N₁₃O₈S: 1260.6750. found 1260.6754.

### Example 94: 6-[3-(1,3-benzothiazol-2-ylamino)-4-methyl-6,7-dihydro-5H-pyrido[2,3-c]pyridazin-8-yl]-3-[1-[[3-[2-[10-[(2R)-2-[(1-fluorocyclopropanecarbonyl)amino]-3-[(2S,4R)-4-hydroxy-2-[[4-(4-methylthiazol-5-yl)phenyl]methylcarbamoyl]pyrrolidin-1-yl]-1,1-dimethyl-3-oxo-propyl]sulfanyldecyl-methyl-amino]ethoxy]-5,7-dimethyl-1-adamantyl]methyl]-5-methyl-pyrazol-4-yl]pyridine-2-carboxylic acid

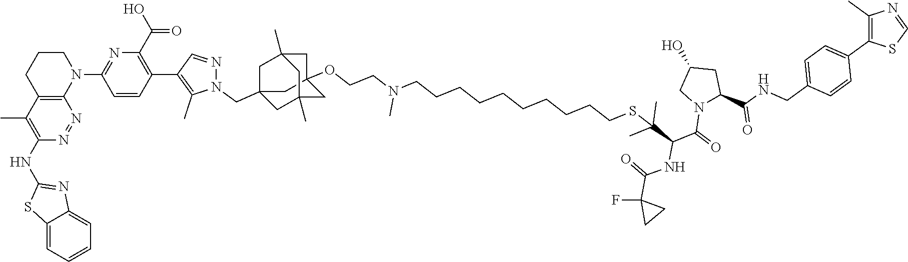 [SMILES: Cc1ncsc1-c1ccc(CNC(=O)[C@@H]2C[C@@H](O)CN2C(=O)[C@@H](NC(=O)C2(F)CC2)C(C)(C)SCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)cc1]

### Step A: (2S,4R)-1-[(2R)-3-(10-bromodecylsulfanyl)-2-[(1-fluorocyclopropanecarbonyl) amino]-3-methyl-butanoyl]-4-hydroxy-N-[[4-(4-methylthiazol-5-yl)phenyl]methyl]pyrrolidine-2-carboxamide

Using General procedure for the alkylation of VHL ligand on thiol group starting from 0.09 mmol of (2S,4R)-1-[(2R)-2-[(1-fluorocyclopropanecarbonyl)amino]-3-methyl-3-sulfanyl-butanoyl]-4-hydroxy-N-[[4-(4-methylthiazol-5-yl)phenyl]methyl]pyrrolidine-2-carboxamide and 1,10-dibromodecane as the appropriate reactant, 36 mg of the desired product were obtained. ¹H NMR (500 MHz, DMSO-d₆) δ ppm 8.98 (s, 1H), 8.58 (t, 1H), 7.48 (dd, 1H), 7.42 (dm, 2H), 7.39 (dm, 2H), 5.19 (d, 1H), 4.78 (d, 1H), 4.46 (t, 1H), 4.43/4.23 (dd+dd, 2H), 4.36 (br., 1H), 3.72/3.64 (dd+dd, 2H), 3.50 (t, 2H), 2.52 (t, 2H), 2.45 (s, 3H), 2.08/1.91 (m+m, 2H), 1.76 (m, 2H), 1.44-1.13 (m, 18H), 1.38/1.35 (s+s, 6H), 1.37/1.21 (m+m, 4H); ¹³C NMR (125 MHz, DMSO-d₆) δ ppm 172.0, 168.6, 168.3, 151.9, 129.2, 127.9, 78.5, 69.3, 59.5, 57.0, 55.3, 49.5, 42.1, 38.4, 35.7, 32.7, 28.2, 27.2/24.7, 16.4, 13.4; HRMS-ESI (m/z): [M+H]⁺ calcd for C₃₅H₅₁BrFN₄O₄S₂: 753.2514. found: 753.2516.

### Step B: 6-[3-(1,3-benzothiazol-2-ylamino)-4-methyl-6,7-dihydro-5H-pyrido[2,3-c]pyridazin-8-yl]-3-[1-[[3-[2-[10-[(2R)-2-[(1-fluorocyclopropanecarbonyl)amino]-3-[(2S,4R)-4-hydroxy-2-[[4-(4-methylthiazol-5-yl)phenyl]methylcarbamoyl]pyrrolidin-1-yl]-1,1-dimethyl-3-oxo-propyl]sulfanyldecyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (28 mg) and the product of Step A as the appropriate alkylating agent, 22 mg of the desired product were obtained. HRMS-ESI (m/z): [M+2H]²⁺ calcd for C₇₆H₁₀₀FN₁₃O₇S₃: 710.8502. found: 710.8504.

### Example 95: 6-[[6-(1,3-benzothiazol-2-ylamino)-5-methyl-pyridazin-3-yl]-[4-[10-[2-(2,6-dioxo-3-piperidyl)-1,3-dioxo-isoindolin-4-yl]oxydecanoyl-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

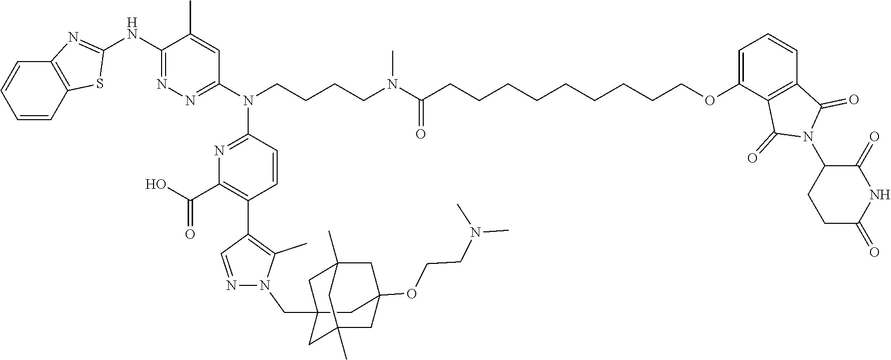 [SMILES: Cc1cc(N(CCCCN(C)C(=O)CCCCCCCCCOc2cccc3c2C(=O)N(C2CCC(=O)NC2=O)C3=O)c2ccc(-c3cnn(CC45CC6(C)CC(C)(C4)CC(OCCN(C)C)(C6)C5)c3C)c(C(=O)O)n2)nnc1Nc1nc2ccccc2s1]

### Step A: 10-[2-(2,6-dioxo-3-piperidyl)-1,3-dioxo-isoindolin-4-yl]oxydecanoic acid

Using General procedure for the alkylation of the 5-hydroxy thalidomide starting from 276 mg (1.00 mmol) of 2-(2,6-dioxo-3-piperidyl)-4-hydroxy-isoindoline-1,3-dione and 10-bromodecanoic acid as the appropriate bromide, 87 mg of the desired product were obtained. ¹H NMR (400 MHz, DMSO-d₆) δ ppm 11.97 (br., 1H), 11.12 (s, 1H), 7.83 (d, 1H), 7.43 (d, 1H), 7.35 (dd, 1H), 5.12 (dd, 1H), 4.17 (t, 2H), 2.89/2.60 (ddd+br., 2H), 2.54/2.05 (m+m, 2H), 2.19 (t, 2H), 1.82-1.20 (m, 14H); ¹³C NMR (100 MHz, DMSO-d₆) δ ppm 175.0, 173.3/170.4, 167.4/167.3, 164.6, 125.8, 121.2, 109.3, 69.3, 49.4, 34.1, 31.4, 22.5; HRMS-ESI (m/z): [M+NH₄]⁺ calcd for C₂₃H₃₂N₃O₇: 462.2235. found: 462.2235.

### Step B: 6-[[6-(1,3-benzothiazol-2-ylamino)-5-methyl-pyridazin-3-yl]-[4-[10-[2-(2,6-dioxo-3-piperidyl)-1,3-dioxo-isoindolin-4-yl]oxydecanoyl-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

Using Degrader Synthesis by Amide Coupling and Hydrolysis General Procedure starting from the product of Step A as the appropriate acid and 40 mg (0.04 mmol) of Preparation 19 as the appropriate amine, 44 mg of the desired product were obtained. HRMS-ESI (m/z): [M+H]⁺ calcd for C₆₇H₈₅N₁₂O₉S: 1233.6278. found 1233.6272.

### Example 96: 6-[3-(1,3-benzothiazol-2-ylamino)-4-methyl-6,7-dihydro-5H-pyrido[2,3-c]pyridazin-8-yl]-3-[1-[[3-[2-[12-[3-[2-[(2S)-1-[(2S)-2-cyclohexyl-2-[[(2S)-2-(methylamino)propanoyl]amino]acetyl]pyrrolidin-2-yl]thiazole-4-carbonyl]phenoxy]dodecyl-methyl-amino]ethoxy]-5,7-dimethyl-1-adamantyl]methyl]-5-methyl-pyrazol-4-yl]pyridine-2-carboxylic acid

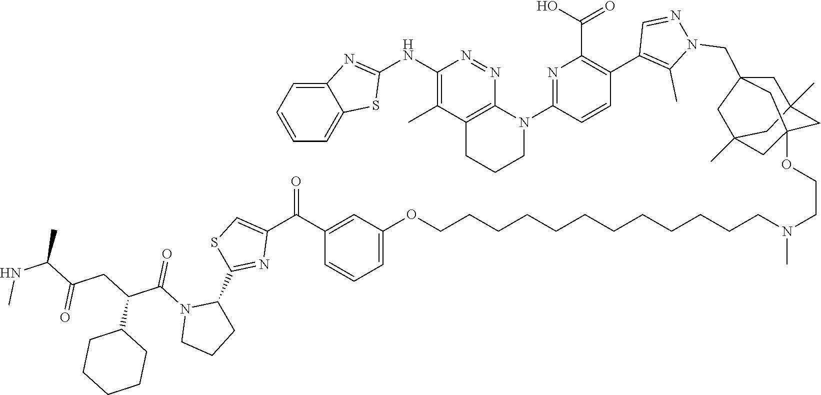 [SMILES: CN[C@@H](C)C(=O)C[C@H](C(=O)N1CCC[C@H]1c1nc(C(=O)c2cccc(OCCCCCCCCCCCCN(C)CCOC34CC5(C)CC(C)(CC(Cn6ncc(-c7ccc(N8CCCc9c8nnc(Nc8nc%10ccccc%10s8)c9C)nc7C(=O)O)c6C)(C5)C3)C4)c2)cs1)C1CCCCC1]

### Step A: tert-butyl N-[(1S)-2-[[(1S)-2-[(2S)-2-[4-[3-(12-bromododecoxy)benzoyl]thiazol-2-yl]pyrrolidin-1-yl]-1-cyclohexyl-2-oxo-ethyl]amino]-1-methyl-2-oxo-ethyl]-N-methyl-carbamate

Using General procedure for the alkylation of IAP ligand starting from tert-butyl N-[(1S)-2-[[(1S)-1-cyclohexyl-2-[(2S)-2-[4-(3-hydroxybenzoyl)thiazol-2-yl]pyrrolidin-1-yl]-2-oxo-ethyl]amino]-1-methyl-2-oxo-ethyl]-N-methyl-carbamate and 60.3 mg (0.18 mmol) of 1,12-dibromododecane as the appropriate dibromoalkane, 67 mg of the desired product were obtained ¹H NMR (500 MHz, dmso-d6) δ ppm 8.49/8.46 (s/s, 1H), 7.83 (br., 1H), 7.66 (m, 1H), 7.65 (d, 1H), 7.45 (t, 1H), 7.23 (dd, 1H), 5.59/5.39 (dd/d, 1H), 4.57/4.48 (br/br., 1H), 4.03 (t, 2H), 3.84-3.73 (m, 2H), 3.51 (t, 2H), 2.75/2.57 (brs/s, 3H), 2.31-2.14 (m, 2H), 2.13-1.97 (m, 2H), 1.38 (brs., 9H), 1.22 (br., 3H); ¹³C NMR (125 MHz, dmso-d6) δ ppm 186.3, 173.2, 130.4, 130.0, 122.7, 119.9, 116.1, 68.1, 59.5/58.6, 53.7/53.2, 47.5, 35.7, 31.9, 30.5/30.0, 28.5, 24.6, 16.1/14.9; HRMS-ESI (m/z): [M+H]⁺ calcd for C₄₃H₆₅BrN₄O₆S: 845.3881. found 845.3880.

### Step B: 6-[3-(1,3-benzothiazol-2-ylamino)-4-methyl-6,7-dihydro-5H-pyrido[2,3-c]pyridazin-8-yl]-3-[1-[[3-[2-[12-[3-[2-[(2S)-1-[(2S)-2-cyclohexyl-2-[[(2S)-2-(methylamino)propanoyl]amino]acetyl]pyrrolidin-2-yl]thiazole-4-carbonyl]phenoxy]dodecyl-methyl-amino]ethoxy]-5,7-dimethyl-1-adamantyl]methyl]-5-methyl-pyrazol-4-yl]pyridine-2-carboxylic acid

Using Degrader Synthesis by Alkylation and Hydrolysis General Procedure starting from the product of Step A as the appropriate bromine and 50 mg (0.06 mmol) of Preparation 4 as the appropriate amine, 51 mg of the desired product were obtained. HRMS-ESI (m/z): [M+2H]²⁺ calcd for C₇₉H₁₀₅N₁₃O₇S₂: 706.8924. found 706.8922.

### Example 97: 6-[3-(1,3-benzothiazol-2-ylamino)-4-methyl-6,7-dihydro-5H-pyrido[2,3-c]pyridazin-8-yl]-3-[1-[[3-[2-[8-[3-[2-[(2S)-1-[(2S)-2-cyclohexyl-2-[[(2S)-2-(methylamino)propanoyl]amino]acetyl]pyrrolidin-2-yl]thiazole-4-carbonyl]phenoxy]octyl-methyl-amino]ethoxy]-5,7-dimethyl-1-adamantyl]methyl]-5-methyl-pyrazol-4-yl]pyridine-2-carboxylic acid

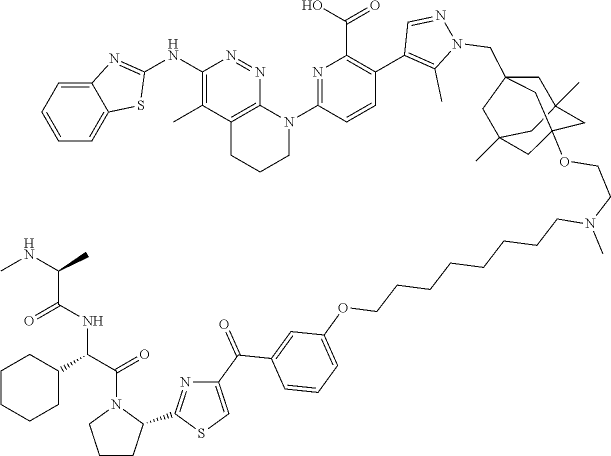 [SMILES: CN[C@@H](C)C(=O)N[C@H](C(=O)N1CCC[C@H]1c1nc(C(=O)c2cccc(OCCCCCCCCN(C)CCOC34CC5(C)CC(C)(CC(Cn6ncc(-c7ccc(N8CCCc9c8nnc(Nc8nc%10ccccc%10s8)c9C)nc7C(=O)O)c6C)(C5)C3)C4)c2)cs1)C1CCCCC1]

### Step A: tert-butyl N-[(1S)-2-[[(1S)-2-[(2S)-2-[4-[3-(8-bromooctoxy)benzoyl]thiazol-2-yl]pyrrolidin-1-yl]-1-cyclohexyl-2-oxo-ethyl]amino]-1-methyl-2-oxo-ethyl]-N-methyl-carbamate

Using General procedure for the alkylation of IAP ligand starting from tert-butyl N-[(1S)-2-[[(1S)-1-cyclohexyl-2-[(2S)-2-[4-(3-hydroxybenzoyl)thiazol-2-yl]pyrrolidin-1-yl]-2-oxo-ethyl]amino]-1-methyl-2-oxo-ethyl]-N-methyl-carbamate and 39.99 mg (0.15 mmol) of 1,8-dibromooctane as the appropriate dibromoalkane, 53 mg of the desired product were obtained. ¹H NMR (500 MHz, dmso-d6) δ ppm 8.49 (s, 1H), 7.84 (br, 1H), 7.66 (t, 1H), 7.65 (dd, 1H), 7.45 (t, 1H), 7.23 (dd, 1H), 5.39 (dd, 1H), 4.57/4.48 (br/br, 1H), 4.42 (t, 1H), 4.03 (t, 2H), 3.79 (t, 2H), 3.52 (t, 2H), 2.75 (s, 3H), 2.25/2.19 (m+m, 2H), 2.04 (qn, 2H), 1.79 (qn, 2H), 1.74 (qn, 2H), 1.70-0.85 (m, 18H), 1.68 (m, 1H), 1.38 (s, 9H), 1.22/1.21 (br/br, 3H); ¹³C NMR (125 MHz, dmso-d6) δ ppm 130.4, 130.0, 122.7, 120.0, 116.0, 66.1, 58.6, 55.2, 54.2/53.2, 47.5, 40.0, 35.7, 32.7, 32.0, 30.5, 29.0, 28.5, 24.6, 16.1/14.9; HRMS-ESI (m/z): [M+H]⁺ calcd for C₃₉H₅₇BrN₄O₆S: 789.3255. found 789.3259.

### Step B: 6-[3-(1,3-benzothiazol-2-ylamino)-4-methyl-6,7-dihydro-5H-pyrido[2,3-c]pyridazin-8-yl]-3-[1-[[3-[2-[8-[3-[2-[(2S)-1-[(2S)-2-cyclohexyl-2-[[(2S)-2-(methylamino)propanoyl]amino]acetyl]pyrrolidin-2-yl]thiazole-4-carbonyl]phenoxy]octyl-methyl-amino]ethoxy]-5,7-dimethyl-1-adamantyl]methyl]-5-methyl-pyrazol-4-yl]pyridine-2-carboxylic acid

Using Degrader Synthesis by Alkylation and Hydrolysis General Procedure starting from the product of Step A as the appropriate bromine and 40 mg (0.05 mmol) of Preparation 4 as the appropriate amine, 18 mg of the desired product were obtained. HRMS-ESI (m/z): [M+H]⁺ calcd for C₇₅H₉₇N₁₃O₇S₂: 1356.7148. found 1356.7140.

### Example 98: 6-[3-(1,3-benzothiazol-2-ylamino)-4-methyl-6,7-dihydro-5H-pyrido[2,3-c]pyridazin-8-yl]-3-[1-[[3-[2-[10-[3-[2-[(2S)-1-[(2S)-2-cyclohexyl-2-[[(2S)-2-(methylamino)propanoyl]amino]acetyl]pyrrolidin-2-yl]thiazole-4-carbonyl]phenoxy]decyl-methyl-amino]ethoxy]-5,7-dimethyl-1-adamantyl]methyl]-5-methyl-pyrazol-4-yl]pyridine-2-carboxylic acid

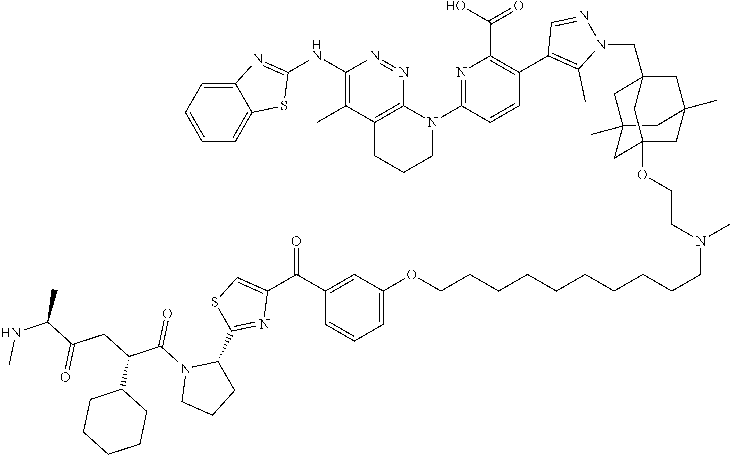

### Step A: tert-butyl N-[(1S)-2-[[(1S)-2-[(2S)-2-[4-[3-(10-bromodecoxy)benzoyl]thiazol-2-yl]pyrrolidin-1-yl]-1-cyclohexyl-2-oxo-ethyl]amino]-1-methyl-2-oxo-ethyl]-N-methyl-carbamate

Using General procedure for the alkylation of IAP ligand starting from tert-butyl N-[(1S)-2-[[(1S)-1-cyclohexyl-2-[(2S)-2-[4-(3-hydroxybenzoyl)thiazol-2-yl]pyrrolidin-1-yl]-2-oxo-ethyl]amino]-1-methyl-2-oxo-ethyl]-N-methyl-carbamate and 44.1 mg (0.15 mmol) of 1,10-dibromodecane as the appropriate dibromoalkane, 36 mg of the desired product were obtained. ¹H NMR (400 MHz, dmso-d6) δ ppm 8.43 (s, 1H), 7.67 (m, 1H), 7.66 (dm, 1H), 7.44 (t, 1H), 7.40 (br., 1H), 7.22 (dm, 1H), 5.41 (dd, 1H), 4.54 (q, 1H), 4.47 (m, 1H), 4.05 (t, 2H), 3.83/3.76 (m+m, 2H), 3.50 (t, 2H), 2.76 (s, 3H), 2.29/2.22 (m+m, 2H), 2.14-1.99 (m, 2H), 1.85-0.87 (m, 1OH), 1.85-0.87 (m, 16H), 1.70 (m, 1H), 1.41 (s, 9H), 1.24 (d, 3H); ¹³C NMR (100 MHz, dmso-d6) δ ppm 186.3, 171.9, 170.9, 155.6, 129.9, 129.8, 122.7, 120.0, 116.3, 68.4, 58.6, 55.2, 54.0, 47.5, 40.3, 35.4, 31.9, 30.5, 28.6, 24.6, 15.2; HRMS-ESI (m/z): [M+H]⁺ calcd for C₄₁H₆₁BrN₄O₆S: 817.3568. found 817.3568.

### Step B: 6-[3-(1,3-benzothiazol-2-ylamino)-4-methyl-6,7-dihydro-5H-pyrido[2,3-c]pyridazin-8-yl]-3-[1-[[3-[2-[10-[3-[2-[(2S)-1-[(2S)-2-cyclohexyl-2-[[(2S)-2-(methylamino)propanoyl]amino]acetyl]pyrrolidin-2-yl]thiazole-4-carbonyl]phenoxy]decyl-methyl-amino]ethoxy]-5,7-dimethyl-1-adamantyl]methyl]-5-methyl-pyrazol-4-yl]pyridine-2-carboxylic acid

Using Degrader Synthesis by Alkylation and Hydrolysis General Procedure starting from the product of Step A as the appropriate bromine and 30 mg (0.04 mmol) of Preparation 4 as the appropriate amine, 21 mg of the desired product were obtained. HRMS-ESI (m/z): [M+2H]²⁺ calcd for C₇₇H₁₀₁N₁₃O₇S₂: 692.8767. found 692.8769.

### Example 99: 6-[3-(1,3-benzothiazol-2-ylamino)-4-methyl-6,7-dihydro-5H-pyrido[2,3-c]pyridazin-8-yl]-3-[1-[[3-[2-[4-[3-[2-[(2S)-1-[(2S)-2-cyclohexyl-2-[[(2S)-2-(methylamino)propanoyl]amino]acetyl]pyrrolidin-2-yl]thiazole-4-carbonyl]phenoxy]butyl-methyl-amino]ethoxy]-5,7-dimethyl-1-adamantyl]methyl]-5-methyl-pyrazol-4-yl]pyridine-2-carboxylic acid

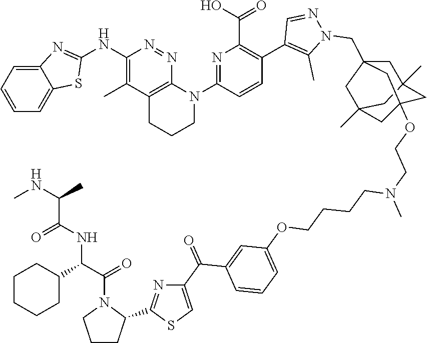 [SMILES: CN[C@@H](C)C(=O)N[C@H](C(=O)N1CCC[C@H]1c1nc(C(=O)c2cccc(OCCCCN(C)CCOC34CC5(C)CC(C)(CC(Cn6ncc(-c7ccc(N8CCCc9c8nnc(Nc8nc%10ccccc%10s8)c9C)nc7C(=O)O)c6C)(C5)C3)C4)c2)cs1)C1CCCCC1]

### Step A: tert-butyl N-[(1S)-2-[[(1S)-2-[(2S)-2-[4-[3-(4-bromobutoxy)benzoyl]thiazol-2-yl]pyrrolidin-1-yl]-1-cyclohexyl-2-oxo-ethyl]amino]-1-methyl-2-oxo-ethyl]-N-methyl-carbamate

Using General procedure for the alkylation of IAP ligand starting from tert-butyl N-[(1S)-2-[[(1S)-1-cyclohexyl-2-[(2S)-2-[4-(3-hydroxybenzoyl)thiazol-2-yl]pyrrolidin-1-yl]-2-oxo-ethyl]amino]-1-methyl-2-oxo-ethyl]-N-methyl-carbamate and 59.5 mg (0.28 mmol) of 1,4-dibromobutane as the appropriate dibromoalkane, 168 mg of the desired product were obtained. ¹H NMR (500 MHz, dmso-d6) δ ppm 8.49 (s, 1H), 7.66 (t, 1H), 7.65 (dd, 1H), 7.46 (t, 1H), 7.24 (dd, 1H), 5.39 (dd, 1H), 4.57/4.48 (br/br, 1H), 4.43 (t, 1H), 4.08 (t, 2H), 3.79 (t, 2H), 3.62 (t, 2H), 2.75 (s, 3H), 2.26/2.19 (m+m, 2H), 2.04 (m, 2H), 1.99 (qn, 2H), 1.87 (qn, 2H), 1.67 (m, 1H), 1.63-1.08 (m, 10H), 1.39 (s, 9H), 1.22 (br, 3H); ¹³C NMR (125 MHz, dmso-d6) δ ppm 130.4, 130.0, 122.9, 119.9, 116.0, 67.4, 58.6, 55.2, 54.1/53.3, 47.5, 39.9, 35.3, 31.9, 30.5, 29.6, 28.5, 27.8, 24.6, 16.1/14.9; HRMS-ESI (m/z): [M+H]⁺ calcd for C₃₅H₄₉BrN₄O₆S: 733.2629. found 733.2621.

### Step B: 6-[3-(1,3-benzothiazol-2-ylamino)-4-methyl-6,7-dihydro-5H-pyrido[2,3-c]pyridazin-8-yl]-3-[1-[[3-[2-[4-[3-[2-[(2S)-1-[(2S)-2-cyclohexyl-2-[[(2S)-2-(methylamino)propanoyl]amino]acetyl]pyrrolidin-2-yl]thiazole-4-carbonyl]phenoxy]butyl-methyl-amino]ethoxy]-5,7-dimethyl-1-adamantyl]methyl]-5-methyl-pyrazol-4-yl]pyridine-2-carboxylic acid

Using Degrader Synthesis by Alkylation and Hydrolysis General Procedure starting from the product of Step A as the appropriate bromine and 40 mg (0.05 mmol) of Preparation 4 as the appropriate amine, 43 mg of the desired product were obtained. HRMS-ESI (m/z): [M+2H]²⁺ calcd for C₇₁H₈₉N₁₃O₇S₂: 650.8300. found 650.8304.

### Example 100: 6-[3-(1,3-benzothiazol-2-ylamino)-4-methyl-6,7-dihydro-5H-pyrido[2,3-c]pyridazin-8-yl]-3-[1-[[3-[2-[14-[3-[2-[(2S)-1-[(2S)-2-cyclohexyl-2-[[(2S)-2-(methylamino)propanoyl]amino]acetyl]pyrrolidin-2-yl]thiazole-4-carbonyl]phenoxy]tetradecyl-methyl-amino]ethoxy]-5,7-dimethyl-1-adamantyl]methyl]-5-methyl-pyrazol-4-yl]pyridine-2-carboxylic acid

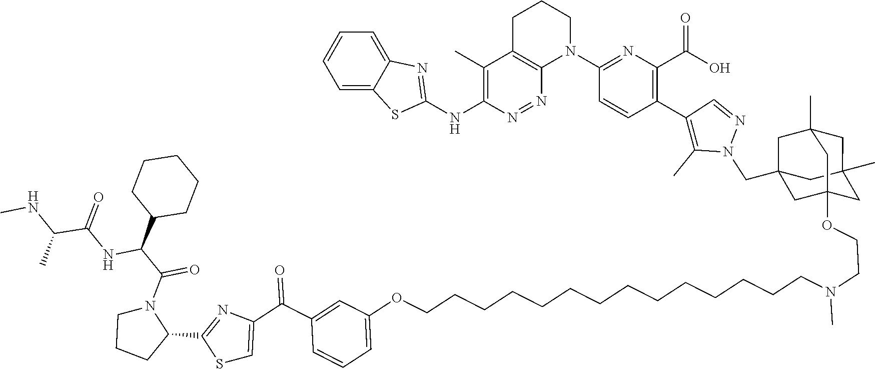

### Step A: tert-butyl N-[(1S)-2-[[(1S)-2-[(2S)-2-[4-[3-(14-bromotetradecoxy)benzoyl]thiazol-2-yl]pyrrolidin-1-yl]-1-cyclohexyl-2-oxo-ethyl]amino]-1-methyl-2-oxo-ethyl]-N-methyl-carbamate

Using General procedure for the alkylation of IAP ligand starting from tert-butyl N-[(1S)-2-[[(1S)-1-cyclohexyl-2-[(2S)-2-[4-(3-hydroxybenzoyl)thiazol-2-yl]pyrrolidin-1-yl]-2-oxo-ethyl]amino]-1-methyl-2-oxo-ethyl]-N-methyl-carbamate and 98.2 mg (0.28 mmol) of 1,14-dibromotetradecane as the appropriate dibromoalkane, 127 mg of the desired product were obtained.

### Step B: 6-[3-(1,3-benzothiazol-2-ylamino)-4-methyl-6,7-dihydro-5H-pyrido[2,3-c]pyridazin-8-yl]-3-[1-[[3-[2-[14-[3-[2-[(2S)-1-[(2S)-2-cyclohexyl-2-[[(2S)-2-(methylamino)propanoyl]amino]acetyl]pyrrolidin-2-yl]thiazole-4-carbonyl]phenoxy]tetradecyl-methyl-amino]ethoxy]-5,7-dimethyl-1-adamantyl]methyl]-5-methyl-pyrazol-4-yl]pyridine-2-carboxylic acid

Using Degrader Synthesis by Alkylation and Hydrolysis General Procedure starting from the product of Step A as the appropriate bromine and 40 mg (0.05 mmol) of Preparation 4 as the appropriate amine, 48 mg of the desired product were obtained. HRMS-ESI (m/z): [M+2H]²⁺ calcd for C₈₁H₁₀₉N₁₃O₇S₂: 720.9080. found 720.9083.

### Example 101: 6-[3-(1,3-benzothiazol-2-ylamino)-4-methyl-6,7-dihydro-5H-pyrido[2,3-c]pyridazin-8-yl]-3-[1-[[3-[2-[2-[3-[2-[(2S)-1-[(2S)-2-cyclohexyl-2-[[(2S)-2-(methylamino)propanoyl]amino]acetyl]pyrrolidin-2-yl]thiazole-4-carbonyl]phenoxy]ethyl-methyl-amino]ethoxy]-5,7-dimethyl-1-adamantyl]methyl]-5-methyl-pyrazol-4-yl]pyridine-2-carboxylic acid

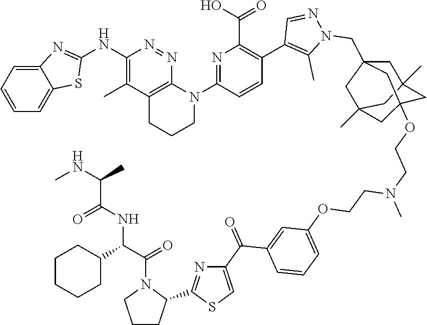 [SMILES: CN[C@@H](C)C(=O)N[C@H](C(=O)N1CCC[C@H]1c1nc(C(=O)c2cccc(OCCN(C)CCOC34CC5(C)CC(C)(CC(Cn6ncc(-c7ccc(N8CCCc9c8nnc(Nc8nc%10ccccc%10s8)c9C)nc7C(=O)O)c6C)(C5)C3)C4)c2)cs1)C1CCCCC1]

### Step A: tert-butyl N-[(1S)-2-[[(1S)-2-[(2S)-2-[4-[3-(2-bromoethoxy)benzoyl]thiazol-2-yl]pyrrolidin-1-yl]-1-cyclohexyl-2-oxo-ethyl]amino]-1-methyl-2-oxo-ethyl]-N-methyl-carbamate

Using General procedure for the alkylation of IAP ligand starting from tert-butyl N-[(1S)-2-[[(1S)-1-cyclohexyl-2-[(2S)-2-[4-(3-hydroxybenzoyl)thiazol-2-yl]pyrrolidin-1-yl]-2-oxo-ethyl]amino]-1-methyl-2-oxo-ethyl]-N-methyl-carbamate and 784.3 mg (0.50 mmol) of 1,2-dibromoethane as the appropriate dibromoalkane, 72 mg of the desired product were obtained. HRMS-ESI (m/z): [M+H]⁺ calcd for C₃₃H₄₅BrN₄O₆S: 705.2316. found 705.2314.

### Step B: 6-[3-(1,3-benzothiazol-2-ylamino)-4-methyl-6,7-dihydro-5H-pyrido[2,3-c]pyridazin-8-yl]-3-[1-[[3-[2-[2-[3-[2-[(2S)-1-[(2S)-2-cyclohexyl-2-[[(2S)-2-(methylamino)propanoyl]amino]acetyl]pyrrolidin-2-yl]thiazole-4-carbonyl]phenoxy]ethyl-methyl-amino]ethoxy]-5,7-dimethyl-1-adamantyl]methyl]-5-methyl-pyrazol-4-yl]pyridine-2-carboxylic acid

Using Degrader Synthesis by Alkylation and Hydrolysis General Procedure starting from the product of Step A as the appropriate bromine and 40 mg (0.05 mmol) of Preparation 4 as the appropriate amine, 34 mg of the desired product were obtained. HRMS-ESI (m/z): [M+2H]²⁺ calcd for C₆₉H₈₅N₁₃O₇S₂: 636.8141. found 636.8144.

### Example 102: 6-[3-(1,3-benzothiazol-2-ylamino)-4-methyl-6,7-dihydro-5H-pyrido[2,3-c]pyridazin-8-yl]-3-[1-[[3-[2-[6-[3-[2-[(2S)-1-[(2S)-2-cyclohexyl-2-[[(2S)-2-(methylamino)propanoyl]amino]acetyl]pyrrolidin-2-yl]thiazole-4-carbonyl]phenoxy]hexyl-methyl-amino]ethoxy]-5,7-dimethyl-1-adamantyl]methyl]-5-methyl-pyrazol-4-yl]pyridine-2-carboxylic acid

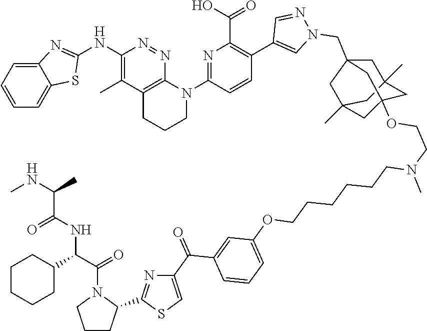 [SMILES: CN[C@@H](C)C(=O)N[C@H](C(=O)N1CCC[C@H]1c1nc(C(=O)c2cccc(OCCCCCCN(C)CCOC34CC5(C)CC(C)(CC(Cn6cc(-c7ccc(N8CCCc9c8nnc(Nc8nc%10ccccc%10s8)c9C)nc7C(=O)O)cn6)(C5)C3)C4)c2)cs1)C1CCCCC1]

### Step A: tert-butyl N-[(1S)-2-[[(1S)-2-[(2S)-2-[4-[3-(6-bromohexoxy)benzoyl]thiazol-2-yl]pyrrolidin-1-yl]-1-cyclohexyl-2-oxo-ethyl]amino]-1-methyl-2-oxo-ethyl]-N-methyl-carbamate

Using General procedure for the alkylation of IAP ligand starting from tert-butyl N-[(1S)-2-[[(1S)-1-cyclohexyl-2-[(2S)-2-[4-(3-hydroxybenzoyl)thiazol-2-yl]pyrrolidin-1-yl]-2-oxo-ethyl]amino]-1-methyl-2-oxo-ethyl]-N-methyl-carbamate and 44.8 mg (0.88 mmol) of 1,6-dibromohexane as the appropriate dibromoalkane, 65 mg of the desired product were obtained. ¹H NMR (500 MHz, dmso-d6) δ ppm 8.43 (s, 1H), 7.67 (m, 1H), 7.66 (dm, 1H), 7.45 (t, 1H), 7.22 (dm, 1H), 5.41 (dd, 1H), 4.54 (q, 1H), 4.47 (t, 1H), 4.06 (t, 2H), 3.82/3.76 (m+m, 2H), 3.53 (t, 2H), 2.76 (s, 3H), 2.34-0.78 (m, 22H), 1.70 (m, 1H), 1.41 (s, 9H), 1.24 (d, 3H); ¹³C NMR (125 MHz, dmso-d6) δ ppm 129.9, 129.8, 122.8, 120.0, 116.2, 68.3, 58.6, 55.2, 54.1, 47.5, 40.3, 35.1, 30.5, 28.6, 15.2; HRMS-ESI (m/z): [M+H]⁺ calcd for C₃₇H₅₃BrN₄O₆S: 761.2942. found 761.2946.

### Step B: 6-[3-(1,3-benzothiazol-2-ylamino)-4-methyl-6,7-dihydro-5H-pyrido[2,3-c]pyridazin-8-yl]-3-[1-[[3-[2-[6-[3-[2-[(2S)-1-[(2S)-2-cyclohexyl-2-[[(2S)-2-(methylamino)propanoyl]amino]acetyl]pyrrolidin-2-yl]thiazole-4-carbonyl]phenoxy]hexyl-methyl-amino]ethoxy]-5,7-dimethyl-1-adamantyl]methyl]-5-methyl-pyrazol-4-yl]pyridine-2-carboxylic acid

Using Degrader Synthesis by Alkylation and Hydrolysis General Procedure starting from the product of Step A as the appropriate bromine and 40 mg (0.05 mmol) of Preparation 4 as the appropriate amine, 47 mg of the desired product were obtained. HRMS-ESI (m/z): [M+2H]²⁺ calcd for C₇₃H₉₃N₁₃O₇S₂: 664.8454. found 664.8458.

### Example 103: 6-[3-(1,3-benzothiazol-2-ylamino)-4-methyl-6,7-dihydro-5H-pyrido[2,3-c]pyridazin-8-yl]-3-[1-[[3-[2-[2-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]ethyl-methyl-amino]ethoxy]-5,7-dimethyl-1-adamantyl]methyl]-5-methyl-pyrazol-4-yl]pyridine-2-carboxylic acid

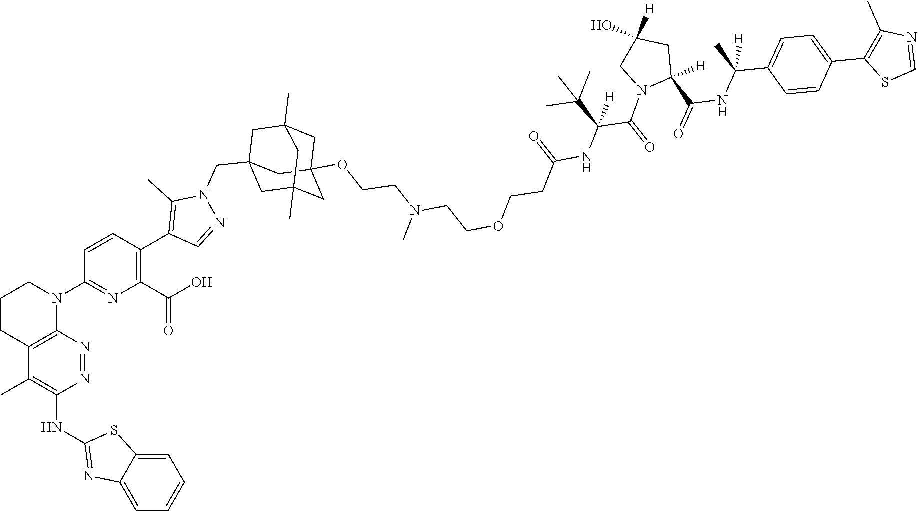 [SMILES: Cc1ncsc1-c1ccc([C@H](C)NC(=O)[C@@H]2C[C@@H](O)CN2C(=O)[C@@H](NC(=O)CCOCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1]

### Step A: tert-butyl 3-[2-(p-tolylsulfonyloxy)ethoxy]propanoate

Using General procedure for the tosylation of the hydroxyalkyl VHL ligand-derivatives starting from 1.5 g of tert-butyl 3-(2-hydroxyethoxy)propanoate (7.88 mmol), 2.3 g of the desired product were obtained. ¹H NMR (400 MHz, dmso-d6) δ ppm 7.78 (d, 2H), 7.48 (d, 2H), 4.10 (t, 2H), 3.55 (t, 2H), 3.52 (t, 2H), 2.43 (s, 3H), 2.36 (t, 2H), 1.38 (s, 9H); ¹³C NMR (100 MHz, dmso-d6) δ ppm 170.7, 145.4, 132.8, 130.6, 128.1, 80.3, 70.3, 68.1, 66.6, 36.1, 28.2, 21.6; HRMS-ESI (m/z): [M+NH4]⁺ calcd for C₁₆H₂₄O₆S·H₄N: 362.1632. found 362.1633.

### Step B: 3-[2-(p-tolylsulfonyloxy)ethoxy]propanoic acid

The product of Step A (1 g, 2.9 mmol) in DCM (14.5 mL) was treated with TFA (7.5 eq) at 0° C. and the mixture was stirred at room temperature until complete conversion was observed. The product was concentrated and used without further purification (802 mg, 95%). ¹H NMR (400 MHz, dmso-d6) δ ppm 12.07 (brs, 1H), 7.78 (d, 2H), 7.48 (d, 2H), 4.09 (t, 2H), 3.54 (t, 2H), 3.52 (t, 2H), 2.42 (s, 3H), 2.37 (t, 2H); ¹³C NMR (100 MHz, dmso-d6) δ ppm 172.9, 145.4, 132.9, 130.6, 128.1, 70.4, 68.2, 66.6, 35.0, 21.6; HRMS-ESI (m/z) [M+H]⁺ calcd for C₁₂H₁₆O₆S: 289.0740. found 289.0740.

### Step C: 2-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]ethyl 4-methylbenzenesulfonate

Using General procedure for the acylation and deprotection of VHL ligands without the hydrolysis step, starting from 300 mg of (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.62 mmol) and the product of Step B as the appropriate acid, 361 mg of the desired product were obtained. ¹H NMR (500 MHz, dmso-d6) δ ppm 8.98 (s, 1H), 8.38 (d, 1H), 7.87 (d, 1H), 7.78 (dm, 2H), 7.48 (dm, 2H), 7.44 (dm, 2H), 7.38 (dm, 2H), 5.11 (br., 1H), 4.91 (m, 1H), 4.51 (d, 1H), 4.42 (t, 1H), 4.27 (br., 1H), 4.08 (t, 2H), 3.65-3.47 (m, 6H), 2.49/2.30 (m+m, 2H), 2.45 (s, 3H), 2.42 (s, 3H), 2.01/1.78 (m+m, 2H), 1.37 (d, 3H), 0.91 (s, 9H); ¹³C NMR (125 MHz, dmso-d6) δ ppm 171.1, 170.2, 169.9, 150.6, 130.6, 129.3, 128.1, 126.9, 70.3, 69.2, 59.0, 56.8, 48.2, 38.2, 35.9, 26.9, 22.9, 21.6, 16.5; HRMS-ESI (m/z): [M+H]⁺ calcd for C₃₅H₄₆N₄O₈S₂: 715.2830. found: 715.2830.

### Step D: 6-[3-(1,3-benzothiazol-2-ylamino)-4-methyl-6,7-dihydro-5H-pyrido[2,3-c]pyridazin-8-yl]-3-[1-[[3-[2-[2-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]ethyl-methyl-amino]ethoxy]-5,7-dimethyl-1-adamantyl]methyl]-5-methyl-pyrazol-4-yl]pyridine-2-carboxylic acid

Using General procedure for production of VHL ligand-based degraders via alkylation starting from the product of Step C as the appropriate alkylating agent and 50 mg (0.06 mmol) of Preparation 4 as the appropriate amine and hydrolysis by a treatment with TFA (125 eq) in DCM (2 mL), 20 mg of the desired product were obtained. HRMS-ESI (m/z): [M+2H]²⁺ calcd for C₆₉H₈₉N₁₃O₈S₂: 645.8193. found: 645.8197.

### Example 104: 6-[3-(1,3-benzothiazol-2-ylamino)-4-methyl-6,7-dihydro-5H-pyrido[2,3-c]pyridazin-8-yl]-3-[1-[[3-[2-[9-[2-(2,6-dioxo-3-piperidyl)-1,3-dioxo-isoindolin-5-yl]oxynonyl-methyl-amino]ethoxy]-5,7-dimethyl-1-adamantyl]methyl]-5-methyl-pyrazol-4-yl]pyridine-2-carboxylic acid

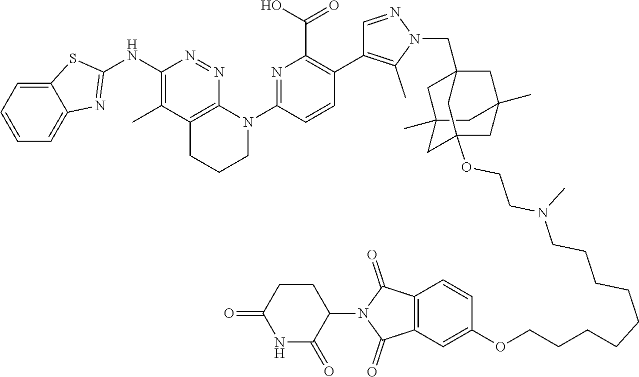 [SMILES: Cc1c(Nc2nc3ccccc3s2)nnc2c1CCCN2c1ccc(-c2cnn(CC34CC5(C)CC(C)(C3)CC(OCCN(C)CCCCCCCCCOc3ccc6c(c3)C(=O)N(C3CCC(=O)NC3=O)C6=O)(C5)C4)c2C)c(C(=O)O)n1]

### Step A: 5-(9-bromononoxy)-2-(2,6-dioxo-3-piperidyl)isoindoline-1,3-dione

Using General procedure for the alkylation of the 5-hydroxy thalidomide starting from 150 mg of 2-(2,6-dioxo-3-piperidyl)-5-hydroxy-isoindoline-1,3-dione (0.54 mmol) and 1,9-dibromononane as the appropriate dibromide, 140 mg of the desired product were obtained. ¹H NMR (400 MHz, dmso-d6) δ ppm 11.12 (s, 1H), 7.84 (d, 1H), 7.43 (d, 1H), 7.35 (dd, 1H), 5.13 (dd, 1H), 4.17 (t, 2H), 3.53 (t, 2H), 2.90/2.60 (td+dd, 2H), 2.53/2.05 (dd+dt, 2H), 1.79 (qn, 2H), 1.76 (qn, 2H), 1.43 (qn, 2H), 1.39 (qn, 2H), 1.36-1.27 (m, 6H); ¹³C NMR (100 MHz, dmso-d6) δ ppm 173.3, 170.4, 167.4, 167.3, 164.6, 134.5, 125.8, 123.4, 121.2, 109.3, 69.3, 49.4, 35.7, 32.7, 31.4, 28.8, 28.0, 25.8, 22.5; HRMS-ESI (m/z): [M+H]⁺ calcd for C₂₂H₂₇BrN₂O₅: 479.1176. found 479.1177.

### Step B: 6-[3-(1,3-benzothiazol-2-ylamino)-4-methyl-6,7-dihydro-5H-pyrido[2,3-c]pyridazin-8-yl]-3-[1-[[3-[2-[9-[2-(2,6-dioxo-3-piperidyl)-1,3-dioxo-isoindolin-5-yl]oxynonyl-methyl-amino]ethoxy]-5,7-dimethyl-1-adamantyl]methyl]-5-methyl-pyrazol-4-yl]pyridine-2-carboxylic acid

Using Degrader Synthesis by Alkylation and Hydrolysis General Procedure starting from the product of Step A as the appropriate bromide and 50 mg (0.06 mmol) of Preparation 4 as the appropriate amine, 44 mg of the desired product were obtained. HRMS-ESI (m/z): [M+H]⁺ calcd for C₆₃H₇₅N₁₁O₈S: 1146.5593. found: 1146.5594.

### Example 105: 6-[3-(1,3-benzothiazol-2-ylamino)-4-methyl-6,7-dihydro-5H-pyrido[2,3-c]pyridazin-8-yl]-3-[1-[[3-[2-[11-[2-(2,6-dioxo-3-piperidyl)-1,3-dioxo-isoindolin-5-yl]oxyundecyl-methyl-amino]ethoxy]-5,7-dimethyl-1-adamantyl]methyl]-5-methyl-pyrazol-4-yl]pyridine-2-carboxylic acid

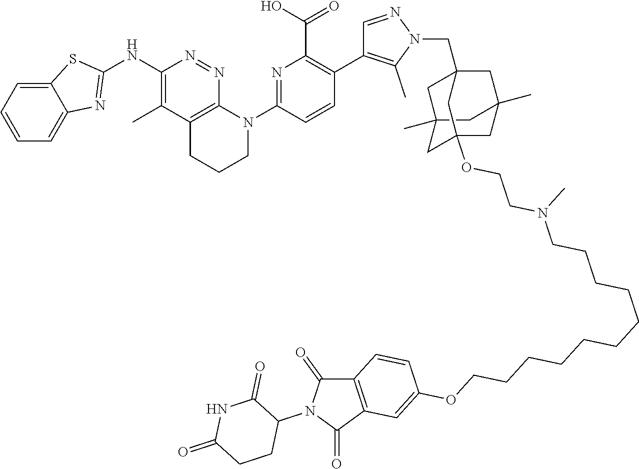 [SMILES: Cc1c(Nc2nc3ccccc3s2)nnc2c1CCCN2c1ccc(-c2cnn(CC34CC5(C)CC(C)(C3)CC(OCCN(C)CCCCCCCCCCCOc3ccc6c(c3)C(=O)N(C3CCC(=O)NC3=O)C6=O)(C5)C4)c2C)c(C(=O)O)n1]

### Step A: 5-(11-bromoundecoxy)-2-(2,6-dioxo-3-piperidyl)isoindoline-1,3-dione

Using General procedure for the alkylation of the 5-hydroxy thalidomide starting from 150 mg of 2-(2,6-dioxo-3-piperidyl)-5-hydroxy-isoindoline-1,3-dione (0.54 mmol) and 1,11-dibromoundecane as the appropriate dibromide, 130 mg of the desired product were obtained. ¹H NMR (400 MHz, dmso-d6) δ ppm 11.12 (s, 1H), 7.83 (d, 1H), 7.42 (d, 1H), 7.34 (dd, 1H), 5.12 (dd, 1H), 4.16 (t, 2H), 3.52 (t, 2H), 2.89/2.60 (m+m, 2H), 2.54/2.04 (m+m, 2H), 1.78 (m, 2H), 1.74 (m, 2H), 1.48-1.20 (m, 14H); ¹³C NMR (100 MHz, dmso-d6) δ ppm 125.8, 121.2, 109.3, 69.3, 49.4, 35.7, 32.7, 31.4, 28.8, 22.6; HRMS-ESI (m/z): [M+H]⁺ calcd for C₂₄H₃₁BrN₂O₅: 507.1489. found 507.1488.

### Step B: 6-[3-(1,3-benzothiazol-2-ylamino)-4-methyl-6,7-dihydro-5H-pyrido[2,3-c]pyridazin-8-yl]-3-[1-[[3-[2-[11-[2-(2,6-dioxo-3-piperidyl)-1,3-dioxo-isoindolin-5-yl]oxyundecyl-methyl-amino]ethoxy]-5,7-dimethyl-1-adamantyl]methyl]-5-methyl-pyrazol-4-yl]pyridine-2-carboxylic acid

Using Degrader Synthesis by Alkylation and Hydrolysis General Procedure starting from the product of Step A as the appropriate bromide and 50 mg (0.06 mmol) of Preparation 4 as the appropriate amine, 49 mg of the desired product were obtained. HRMS-ESI (m/z): [M+H]⁺ calcd for C₆₅H₇₉N₁₁O₈S: 1174.5906. found: 1173.5902.

### Example 106: 6-[3-(1,3-benzothiazol-2-ylamino)-4-methyl-6,7-dihydro-5H-pyrido[2,3-c]pyridazin-8-yl]-3-[1-[[3-[2-[7-[2-(2,6-dioxo-3-piperidyl)-1,3-dioxo-isoindolin-5-yl]oxyheptyl-methyl-amino]ethoxy]-5,7-dimethyl-1-adamantyl]methyl]-5-methyl-pyrazol-4-yl]pyridine-2-carboxylic acid

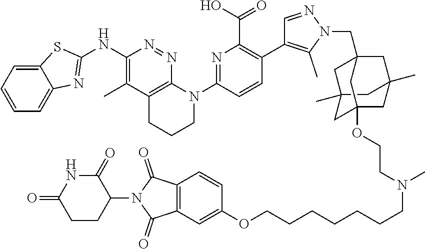 [SMILES: Cc1c(Nc2nc3ccccc3s2)nnc2c1CCCN2c1ccc(-c2cnn(CC34CC5(C)CC(C)(C3)CC(OCCN(C)CCCCCCCOc3ccc6c(c3)C(=O)N(C3CCC(=O)NC3=O)C6=O)(C5)C4)c2C)c(C(=O)O)n1]

### Step A: 5-(7-bromoheptoxy)-2-(2,6-dioxo-3-piperidyl)isoindoline-1,3-dione

Using General procedure for the alkylation of the 5-hydroxy thalidomide starting from 150 mg of 2-(2,6-dioxo-3-piperidyl)-5-hydroxy-isoindoline-1,3-dione (0.54 mmol) and 1,7-dibromoheptane as the appropriate dibromide, 143 mg of the desired product were obtained. ¹H NMR (400 MHz, dmso-d6) 6 ppm 11.12 (s, 1H), 7.84 (d, 1H), 7.43 (d, 1H), 7.36 (dd, 1H), 5.12 (dd, 1H), 4.18 (t, 2H), 3.54 (t, 2H), 2.90/2.60 (td+dd, 2H), 2.54/2.05 (dd+dt, 2H), 1.81 (qn, 2H), 1.76 (qn, 2H), 1.44 (qn, 2H), 1.41 (qn, 2H), 1.38 (qn, 2H); ¹³C NMR (100 MHz, dmso-d6) 6 ppm 125.8, 121.3, 109.3, 69.2, 49.4, 35.7, 32.6, 31.5, 28.7, 28.2, 28.0, 25.7, 22.5; HRMS-ESI (m/z): [M+H]⁺ calcd for C₂₀H₂₃BrN₂O₅: 451.0863. found 451.0867.

### Step B: 6-[3-(1,3-benzothiazol-2-ylamino)-4-methyl-6,7-dihydro-5H-pyrido[2,3-c]pyridazin-8-yl]-3-[1-[[3-[2-[7-[2-(2,6-dioxo-3-piperidyl)-1,3-dioxo-isoindolin-5-yl]oxyheptyl-methyl-amino]ethoxy]-5,7-dimethyl-1-adamantyl]methyl]-5-methyl-pyrazol-4-yl]pyridine-2-carboxylic acid

Using Degrader Synthesis by Alkylation and Hydrolysis General Procedure starting from the product of Step A as the appropriate bromide and 50 mg (0.06 mmol) of Preparation 4 as the appropriate amine, 45 mg of the desired product were obtained. HRMS-ESI (m/z): [M+H]⁺ calcd for C₆₁H₇₁N₁₁O₈S: 1118.5280. found 1118.5288.

### Example 107: 6-[3-(1,3-benzothiazol-2-ylamino)-4-methyl-6,7-dihydro-5H-pyrido[2,3-c]pyridazin-8-yl]-3-[1-[[3-[2-[5-[2-(2,6-dioxo-3-piperidyl)-1,3-dioxo-isoindolin-5-yl]oxypentyl-methyl-amino]ethoxy]-5,7-dimethyl-1-adamantyl]methyl]-5-methyl-pyrazol-4-yl]pyridine-2-carboxylic acid

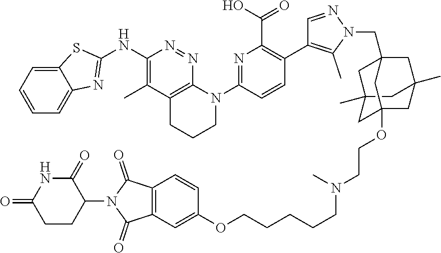 [SMILES: Cc1c(Nc2nc3ccccc3s2)nnc2c1CCCN2c1ccc(-c2cnn(CC34CC5(C)CC(C)(C3)CC(OCCN(C)CCCCCOc3ccc6c(c3)C(=O)N(C3CCC(=O)NC3=O)C6=O)(C5)C4)c2C)c(C(=O)O)n1]

### Step A: 5-(5-bromopentoxy)-2-(2,6-dioxo-3-piperidyl)isoindoline-1,3-dione

Using General procedure for the alkylation of the 5-hydroxy thalidomide starting from 150 mg of 2-(2,6-dioxo-3-piperidyl)-5-hydroxy-isoindoline-1,3-dione (0.54 mmol) and 1,5-dibromopentane as the appropriate dibromide, 141 mg of the desired product were obtained. ¹H NMR (400 MHz, dmso-d6) 6 ppm 11.13 (s, 1H), 7.84 (d, 1H), 7.44 (d, 1H), 7.36 (dd, 1H), 5.13 (dd, 1H), 4.19 (t, 2H), 3.58 (t, 2H), 2.90/2.60 (td+dd, 2H), 2.54/2.05 (dd+dt, 2H), 1.89 (qn, 2H), 1.80 (qn, 2H), 1.56 (qn, 2H); ¹³C NMR (100 MHz, dmso-d6) 6 ppm 125.8, 121.3, 109.3, 69.1, 49.4, 35.6, 32.3, 31.4, 24.6, 22.5, 18.0; HRMS-ESI (m/z): [M+H]⁺ calcd for C₁₈H₁₉BrN₂O₅: 423.0550. found 423.0553.

### Step B: 6-[3-(1,3-benzothiazol-2-ylamino)-4-methyl-6,7-dihydro-5H-pyrido[2,3-c]pyridazin-8-yl]-3-[1-[[3-[2-[5-[2-(2,6-dioxo-3-piperidyl)-1,3-dioxo-isoindolin-5-yl]oxypentyl-methyl-amino]ethoxy]-5,7-dimethyl-1-adamantyl]methyl]-5-methyl-pyrazol-4-yl]pyridine-2-carboxylic acid

Using Degrader Synthesis by Alkylation and Hydrolysis General Procedure starting from the product of Step A as the appropriate bromide and 75 mg (0.09 mmol) of Preparation 4 as the appropriate amine, 42 mg of the desired product were obtained. HRMS-ESI (m/z): [M+H]⁺ calcd for C₅₉H₆₇N₁₁O₈S 1090.4967. found 1090.4970.

### Example 108: 6-[3-(1,3-benzothiazol-2-ylamino)-4-methyl-6,7-dihydro-5H-pyrido[2,3-c]pyridazin-8-yl]-3-[1-[[3-[2-[6-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]hexyl-methyl-amino]ethoxy]-5,7-dimethyl-1-adamantyl]methyl]-5-methyl-pyrazol-4-yl]pyridine-2-carboxylic acid

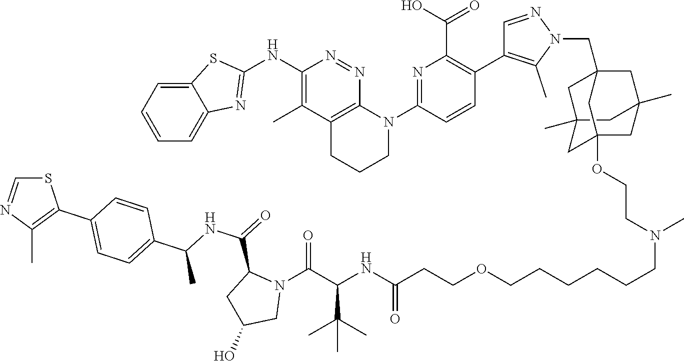 [SMILES: Cc1ncsc1-c1ccc([C@H](C)NC(=O)[C@@H]2C[C@@H](O)CN2C(=O)[C@@H](NC(=O)CCOCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1]

### Step A: tert-butyl 3-(6-hydroxyhexoxy)propanoate

The mixture of 1.0 g of hexane-1,6-diol (8.46 mmol), benzyl(trimethyl)ammonium, hydroxide (1:1) (0.77 mL, 1.69 mmol), and tert-butyl prop-2-enoate (1.5 mL, 10.15 mmol) in 10 mL acetonitrile was stirred for 3 days. The reaction was quenched with brine and extracted with DCM to give 100 mg of the desired product. HPLC-MS [M+Na]⁺ calcd for C₁₃H₂₆NaO₄ 269. found: 269.

### Step B: tert-butyl 3-[6-(p-tolylsulfonyloxy)hexoxy]propanoate

Using General procedure for the tosylation of the hydroxyalkyl VHL ligand-derivatives starting from 285 mg of the product of Step A, 333 mg of the desired product were obtained. ¹H NMR (400 MHz, dmso-d6) 6 ppm 7.78 (dm, 2H), 7.48 (dm, 2H), 3.99 (t, 2H), 3.61 (t, 2H), 3.28 (t, 2H), 2.42 (s, 3H), 2.38 (t, 2H), 1.60-1.10 (m, 8H), 1.38 (s, 9H); ¹³C NMR (100 MHz, dmso-d6) δ ppm 170.9, 145.3, 133.0, 130.6, 128.0, 71.3, 70.3, 66.3, 36.4, 28.2, 21.6; HRMS (ESI) [M+NH4]⁺ calcd for C₂₀H₃₆NO₆S: 418.2258. found 418.2258.

### Step C: 3-[6-(p-tolylsulfonyloxy)hexoxy]propanoic acid

The product of Step B (333 mg) in DCM (4 mL) was treated with TFA (7.5 eq) at 0° C. and the mixture was stirred at room temperature until complete conversion was observed. The product was concentrated and used without further purification (280 mg, 97%). ¹H NMR (400 MHz, dmso-d6) δ ppm 12.14 (br., 1H), 7.78 (dm, 2H), 7.48 (dm, 2H), 3.99 (t, 2H), 3.53 (t, 2H), 3.28 (t, 2H), 2.42 (s, 3H), 2.41 (t, 2H), 1.60-1.10 (m, 8H); ¹³C NMR (100 MHz, dmso-d6) δ ppm 173.2, 145.3, 133.0, 130.6, 128.0, 71.3, 70.3, 66.3, 35.2, 21.6; HRMS-ESI (m/z): [M+H]⁺ calcd for C₁₆H₂₄O₆S: 345.1366. found 345.1367.

### Step D: 6-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]hexyl 4-methylbenzenesulfonate

Using General procedure for the acylation and deprotection of VHL ligands without the hydrolysis step starting from 250 mg of (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.52 mmol) and the product of Step C as the appropriate acid, 130 mg of the desired product were obtained. HRMS-ESI (m/z): [M+H]⁺ calcd for C₃₉H₅₄N₄O₈S₂: 771.3456. found 771.3459.

### Step E: 6-[3-(1,3-benzothiazol-2-ylamino)-4-methyl-6,7-dihydro-5H-pyrido[2,3-c]pyridazin-8-yl]-3-[1-[[3-[2-[6-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]hexyl-methyl-amino]ethoxy]-5,7-dimethyl-1-adamantyl]methyl]-5-methyl-pyrazol-4-yl]pyridine-2-carboxylic acid

Using General procedure for production of VHL ligand-based degraders via alkylation starting from the product of Step D as the appropriate alkylating agent and 60 mg (0.07 mmol) of Preparation 4 as the appropriate amine and the hydrolysis by a treatment with TFA (125 eq) in DCM (2 mL), 36 mg of the desired product were obtained. HRMS-ESI (m/z): [M+H]⁺ calcd for C₇₃H₉₅N₁₃O₈S₂: 1346.6940. found: 1346.6940.

### Example 109: 6-[3-(1,3-benzothiazol-2-ylamino)-4-methyl-6,7-dihydro-5H-pyrido[2,3-c]pyridazin-8-yl]-3-[1-[[3-[2-[9-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]nonyl-methyl-amino]ethoxy]-5,7-dimethyl-1-adamantyl]methyl]-5-methyl-pyrazol-4-yl]pyridine-2-carboxylic acid

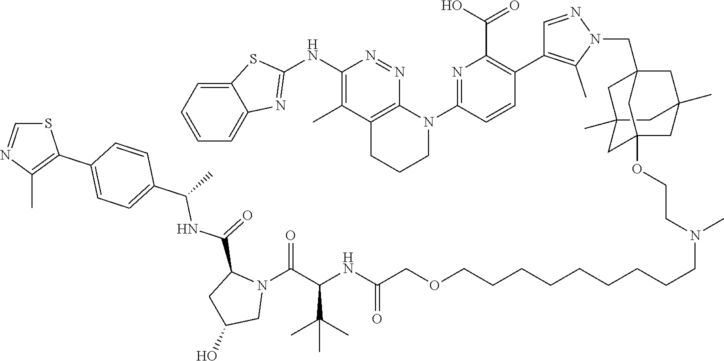 [SMILES: Cc1ncsc1-c1ccc([C@H](C)NC(=O)[C@@H]2C[C@@H](O)CN2C(=O)[C@@H](NC(=O)COCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1]

### Step A: 9-hydroxynonyl 4-methylbenzenesulfonate

Using General procedure for the tosylation of the hydroxyalkyl VHL ligand-derivatives starting from 1.9 g (12 mmol) of nonane-1,9-diol, 860 mg of the desired product were obtained. ¹H NMR (500 MHz, dmso-d6) δ ppm 7.78 (d, 2H), 7.48 (d, 2H), 4.31 (br., 1H), 4.00 (t, 2H), 3.37 (br., 2H), 2.42 (s, 3H), 1.53 (m, 2H), 1.39 (m, 2H), 1.31-1.09 (m, 10H); ¹³C NMR (125 MHz, dmso-d6) δ ppm 145.3, 133.0, 130.6, 128, 71.4, 61.2, 33.0, 28.6, 21.6; HRMS-ESI (m/z): [M+H]⁺ calcd for C₁₆H₂₆04S 315.1624. found: 315.1624.

### Step B: tert-butyl 2-[9-(p-tolylsulfonyloxy)nonoxy]acetate

To 257 mg (0.82 mmol) of the product of Step A in 2 ml of DCM were added diacetoxyrhodium 2.71 mg, (0.01 mmol) and tert-butyl 2-diazoacetate 1.16 mg (1.23 mmol) and the mixture was stirred for 18 h. The product was purified by column chromatography using heptane and ethyl acetate as eluents to give 167 mg of the desired product. ¹H NMR (500 MHz, dmso-d6) δ ppm 7.78 (dm, 2H), 7.48 (dm, 2H), 4.00 (t, 2H), 3.92 (s, 2H), 3.40 (t, 2H), 2.42 (s, 3H), 1.53 (m, 2H), 1.47 (m, 2H), 1.41 (s, 9H), 1.30-1.08 (m, 10H); ¹³C NMR (125 MHz, dmso-d6) δ ppm 170.0, 145.3, 133.0, 130.6, 128.0, 81.0, 71.4, 71.0, 68.4, 29.6, 28.6, 28.2, 21.6; HRMS-ESI: [M+NH4]⁺ calcd for C₂₂H₄₀NO₆S: 446.2571. found 446.2572.

### Step C: 2-[9-(p-tolylsulfonyloxy)nonoxy]acetic acid

The product of Step B (167 mg) in DCM (2 mL) was treated with TFA (7.5 eq) at 0° C. and the mixture was stirred at room temperature until complete conversion was observed. The product was concentrated and used without further purification (142 mg, 97%). ¹H NMR (400 MHz, dmso-d6) δ ppm 12.52 (brs, 1H), 7.78 (m, 2H), 7.48 (m, 2H), 4.00 (t, 2H), 3.95 (s, 2H), 3.41 (t, 2H), 2.42 (s, 3H), 1.53 (m, 2H), 1.47 (m, 2H), 1.33-1.05 (m, 1 OH); ¹³C NMR (100 MHz, dmso-d6) δ ppm 130.6, 128.1, 71.4, 70.9, 67.8, 29.6, 28.5, 21.6; HRMS-ESI (m/z): [M+NH4]⁺ calcd for C₁₈H₃₂NO₆S: 390.1945. found 390.1940.

### Step D: 9-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]nonyl 4-methylbenzenesulfonate

Using General procedure for the acylation and deprotection of VHL ligands without the hydrolysis step starting from 125 mg of (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.26 mmol) and the product of Step C as the appropriate acid, 134 mg of the desired product were obtained. ¹H NMR (500 MHz, dmso-d6) δ ppm 8.98 (s, 1H), 8.45 (d, 1H), 7.78 (dm, 2H), 7.48 (dm, 2H), 7.43 (dm, 2H), 7.36 (dm, 2H), 7.29 (d, 1H), 5.14 (d, 1H), 4.90 (m, 1H), 4.53 (d, 1H), 4.44 (t, 1H), 4.28 (br., 1H), 4.00 (t, 2H), 3.90 (s, 2H), 3.60/3.55 (dd+d, 2H), 3.46 (m, 2H), 2.45 (s, 3H), 2.42 (s, 3H), 2.06/1.76 (m+m, 2H), 1.60-1.07 (m, 14H), 1.36 (d, 3H), 0.91 (s, 9H); ¹³C NMR (125 MHz, dmso-d6) δ ppm 171.1, 168.9, 152.0, 130.7, 129.3, 128.1, 126.8, 71.4, 71.4, 69.9, 69.3, 59.0, 57.0, 56.0.1, 48.2, 38.2, 26.9, 23.0, 21.6, 16.5; HRMS-ESI (m/z): [M+H]⁺ calcd for C₄₁H₅₈N₄O₈S₂ 799.3769. found 799.3774.

### Step E: 6-[3-(1,3-benzothiazol-2-ylamino)-4-methyl-6,7-dihydro-5H-pyrido[2,3-c]pyridazin-8-yl]-3-[1-[[3-[2-[9-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]nonyl-methyl-amino]ethoxy]-5,7-dimethyl-1-adamantyl]methyl]-5-methyl-pyrazol-4-yl]pyridine-2-carboxylic acid

Using General procedure for production of VHL ligand-based degraders via alkylation starting from the product of Step D as the appropriate alkylating agent and 50 mg (0.06 mmol) of Preparation 4 as the appropriate amine and the hydrolysis by a treatment with TFA (125 eq) in DCM (2 mL), 29 mg of the desired product were obtained. HRMS-ESI (m/z): [M+2H]²⁺ calcd for C₇₅H₉₉N₁₃O₈S₂: 687.8663. found: 687.8664.

### Example 110: 6-[3-(1,3-benzothiazol-2-ylamino)-4-methyl-6,7-dihydro-5H-pyrido[2,3-c]pyridazin-8-yl]-3-[1-[[3-[2-[11-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]undecyl-methyl-amino]ethoxy]-5,7-dimethyl-1-adamantyl]methyl]-5-methyl-pyrazol-4-yl]pyridine-2-carboxylic acid

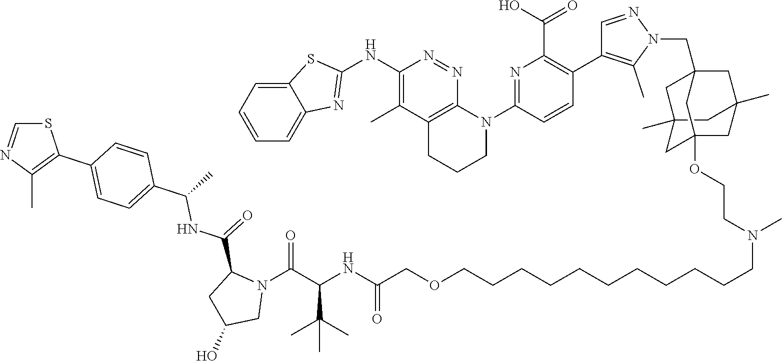 [SMILES: Cc1ncsc1-c1ccc([C@H](C)NC(=O)[C@@H]2C[C@@H](O)CN2C(=O)[C@@H](NC(=O)COCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1]

### Step A: 11-hydroxyundecyl 4-methylbenzenesulfonate

Using General procedure for the tosylation of the hydroxyalkyl VHL ligand-derivatives starting from 2.3 g (12 mmol) of undecane-1,11-diol, 860 mg of the desired product were obtained. ¹H NMR (500 MHz, dmso-d6) δ ppm 7.78 (d, 2H), 7.48 (d, 2H), 4.31 (t, 1H), 4.00 (t, 2H), 3.37 (q, 2H), 2.42 (s, 3H), 1.53 (m, 2H), 1.39 (m, 2H), 1.30-1.08 (m, 14H); ¹³C NMR (125 MHz, dmso-d6) δ ppm 145.3, 133.0, 130.6, 128.0, 71.4, 61.2, 33.0, 28.6, 21.6; HRMS-ESI (m/z): [M+H]⁺ calcd for C₁₃H₂₆O₄ 343.1938. found: 343.1938.

### Step B: tert-butyl 2-[11-(p-tolylsulfonyloxy)undecoxy]acetate

To the product of Step A (274 mg, 0.80 mmol) in 2 mL of dichloromethane were added diacetoxyrhodium (2.65 mg, 0.015 eq) and tert-butyl 2-diazoacetate (1.14 g, 1.2 mmol). Then, the reaction mixture was stirred for 18 h. The product was purified by column chromatography using heptane and ethyl acetate as eluents to give 261 mg of the desired product. ¹H NMR (500 MHz, dmso-d6) δ ppm 7.78 (dm, 2H), 7.48 (dm, 2H), 4.00 (t, 2H), 3.92 (s, 2H), 3.40 (t, 2H), 2.42 (s, 3H), 1.53 (m, 2H), 1.48 (m, 2H), 1.41 (s, 9H), 1.33-1.08 (m, 14H); ¹³C NMR (125 MHz, dmso-d6) δ ppm 170.0, 145.3, 133.0, 130.6, 128.0, 81.0, 71.4, 71.0, 68.5, 29.6, 28.6, 28.2, 21.6; HRMS-ESI (m/z): [M+NH₄]⁺ calcd for C₂₄H₄₄NO₆S: 474.2886. found 474.2886.

### Step C: 2-[11-(p-tolylsulfonyloxy)undecoxy]acetic acid

The product of Step B (261 mg) in DCM (2 mL) was treated with TFA (7.5 eq) at 0° C. and the mixture was stirred at room temperature until complete conversion was observed. The product was concentrated and used without further purification (225 mg). ¹H NMR (400 MHz, dmso-d6) δ ppm 12.58 (brs, 1H), 7.78 (m, 2H), 7.48 (m, 2H), 4.00 (t, 2H), 3.96 (s, 2H), 3.41 (t, 2H), 2.42 (s, 3H), 1.53 (m, 2H), 1.48 (m, 2H), 1.34-1.08 (m, 14H); ¹³C NMR (100 MHz, dmso-d6) δ ppm 130.6, 128.0, 71.4, 70.9, 67.8, 29.5, 28.6, 21.6; HRMS-ESI (m/z): [M+NH₄]⁺ calcd for C₂₀H₃₆NO₆S: 418.2258. found 418.2256.

### Step D: 11-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]undecyl 4-methylbenzenesulfonate

Using General procedure for the acylation and deprotection of VHL ligands without the hydrolysis step starting from 200 mg of (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.42 mmol) and the product of Step C as the appropriate acid, 341 mg of the desired product were obtained. ¹H NMR (500 MHz, dmso-d6) δ ppm 8.98 (s, 1H), 8.45 (d, 1H), 7.78 (dm, 2H), 7.48 (dm, 2H), 7.43 (dm, 2H), 7.36 (dm, 2H), 7.29 (d, 1H), 5.14 (d, 1H), 4.90 (m, 1H), 4.53 (d, 1H), 4.44 (t, 1H), 4.28 (br., 1H), 4.00 (t, 2H), 3.90 (s, 2H), 3.60/3.55 (dd+d, 2H), 3.46 (m, 2H), 2.45 (s, 3H), 2.42 (s, 3H), 2.06/1.76 (m+m, 2H), 1.59-1.07 (m, 18H), 1.37 (d, 3H), 0.91 (s, 9H); ¹³C NMR (125 MHz, dmso-d6) δ ppm 171.1, 168.9, 152.0, 130.7, 129.3, 128.1, 126.8, 71.4, 71.4, 69.9, 69.3, 59.0, 57.0, 56.1, 48.2, 38.2, 26.9, 23, 21.6, 16.5; HRMS-ESI (m/z): [M+H]⁺ calcd for C₄₃H₆₂N₄O₈S₂: 827.4082. found 827.4083.

### Step E: 6-[3-(1,3-benzothiazol-2-ylamino)-4-methyl-6,7-dihydro-5H-pyrido[2,3-c]pyridazin-8-yl]-3-[1-[[3-[2-[11-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]undecyl-methyl-amino]ethoxy]-5,7-dimethyl-1-adamantyl]methyl]-5-methyl-pyrazol-4-yl]pyridine-2-carboxylic acid

Using General procedure for production of VHL ligand-based degraders via alkylation starting from the product of Step D as the appropriate alkylating agent and 40 mg (0.05 mmol) of Preparation 4 as the appropriate amine and the hydrolysis by a treatment with TFA (125 eq) in DCM (2 mL), 12 mg of the desired product were obtained. HRMS-ESI (m/z): [M+2H]²⁺ calcd for C₇₇H₁₀₃N₁₃O₈S₂: 701.8819. found: 701.8818.

### Example 111: 6-[3-(1,3-benzothiazol-2-ylamino)-4-methyl-6,7-dihydro-5H-pyrido[2,3-c]pyridazin-8-yl]-3-[1-[[3-[2-[13-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]tridecyl-methyl-amino]ethoxy]-5,7-dimethyl-1-adamantyl]methyl]-5-methyl-pyrazol-4-yl]pyridine-2-carboxylic acid

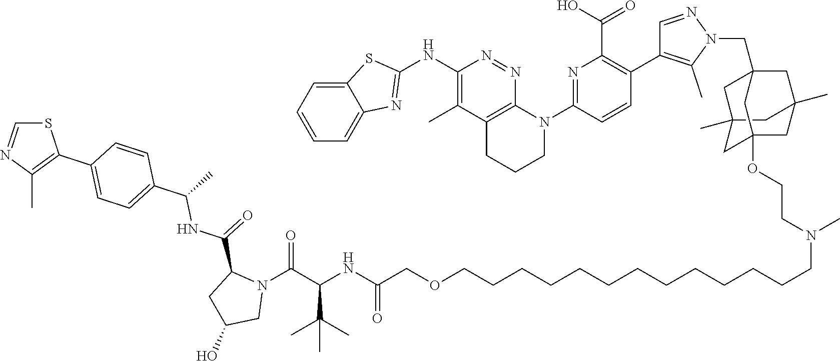 [SMILES: Cc1ncsc1-c1ccc([C@H](C)NC(=O)[C@@H]2C[C@@H](O)CN2C(=O)[C@@H](NC(=O)COCCCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1]

### Step A: 13-hydroxytridecyl 4-methylbenzenesulfonate

Using General procedure for the tosylation of the hydroxyalkyl VHL ligand-derivatives starting from 2.3 g (12.0 mmol) of tridecane-1,13-diol, 870 mg of the desired product were obtained. ¹H NMR (500 MHz, dmso-d6) δ ppm 7.78 (m, 2H), 7.48 (m, 2H), 4.31 (t, 1H), 4.00 (t, 2H), 3.36 (m, 2H), 2.42 (s, 3H), 1.53 (m, 2H), 1.39 (m, 2H), 1.32-1.09 (m, 18H); ¹³C NMR (125 MHz, dmso-d6) δ ppm 130.6, 128.0, 71.4, 61.2, 33.0, 28.6, 21.6; HRMS-ESI [M+H]⁺ calcd for C₂₀H₃₄04S: 371.2250. found: 371.2250.

### Step B: tert-butyl 2-[13-(p-tolylsulfonyloxy)tridecoxy]acetate

To the product of Step A (223 mg, 0.62 mmol) in 2.5 mL of dichloromethane were added diacetoxyrhodium (2.0 mg, 0.015 mmol) and tert-butyl 2-diazoacetate (128 mg, 0.90 mmol). Then, the reaction mixture was stirred for 18 h. The product was purified by column chromatography using heptane and ethyl acetate as eluents to give 145 mg of the desired product. ¹H NMR (500 MHz, dmso-d6) δ ppm 7.78 (dm, 2H), 7.48 (dm, 2H), 4.00 (t, 2H), 3.91 (s, 2H), 3.40 (t, 2H), 2.42 (s, 3H), 1.53 (m, 2H), 1.48 (m, 2H), 1.41 (s, 9H), 1.33-1.08 (m, 18H); ¹³C NMR (125 MHz, dmso-d6) δ ppm 170.0, 145.3, 133.0, 130.6, 128, 81.0, 71.4, 71.0, 68.5, 29.6, 28.6, 28.2, 21.6; HRMS-ESI (m/z): [M+NH4]⁺ calcd for C₂₆H₄₈NO₆S: 502.3197. found 502.3198.

### Step C: 2-[13-(p-tolylsulfonyloxy)tridecoxy]acetic acid

The product of Step B (145 mg) in DCM (2 mL) was treated with TFA (7.5 eq) at 0° C. and the mixture was stirred at room temperature until complete conversion was observed. The product was concentrated and used without further purification (126 mg). ¹H NMR (500 MHz, dmso-d6) δ ppm 12.54 (br., 1H), 7.78 (dm, 2H), 7.48 (dm, 2H), 4.00 (t, 2H), 3.95 (s, 2H), 3.41 (t, 2H), 2.42 (s, 3H), 1.53 (m, 2H), 1.48 (m, 2H), 1.35-1.06 (m, 18H); ¹³C NMR (125 MHz, dmso-d6) δ ppm 172.2, 145.3, 133.0, 130.6, 128.0, 71.4, 70.9, 67.8, 29.6, 28.6, 21.6; HRMS-ESI (m/z): [M+NH₄]⁺ calcd for C₂₂H₃₆O₆S: 446.2571. found 446.2570.

### Step D: 13-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]tridecyl 4-methylbenzenesulfonate

Using General procedure for the acylation and deprotection of VHL ligands without the hydrolysis step starting from 125 mg of (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.26 mmol) and the product of Step C as the appropriate acid, 201 mg of the desired product were obtained. ¹H NMR (500 MHz, dmso-d6) δ ppm 8.98 (s, 1H), 8.45 (d, 1H), 7.78 (dm, 2H), 7.48 (dm, 2H), 7.43 (dm, 2H), 7.36 (dm, 2H), 7.29 (d, 1H), 5.14 (d, 1H), 4.90 (m, 1H), 4.53 (d, 1H), 4.44 (t, 1H), 4.28 (br., 1H), 4.00 (t, 2H), 3.90 (s, 2H), 3.60/3.55 (dd+d, 2H), 3.46 (m, 2H), 2.45 (s, 3H), 2.42 (s, 3H), 2.06/1.76 (m+m, 2H), 1.60-1.07 (m, 22H), 1.37 (d, 3H), 0.91 (s, 9H); ¹³C NMR (125 MHz, dmso-d6) δ ppm 171.1, 168.9, 152.0, 130.7, 129.3, 128.1, 126.8, 71.4, 71.4, 69.9, 69.3, 59.0, 57.0, 56.1, 48.2, 38.2, 26.9, 23.0, 21.6, 16.5; HRMS-ESI (m/z): [M+H]⁺ calcd for C₄₅H₆₆N₄O₈S₂: 855.4395. found: 855.4396.

### Step E: 6-[3-(1,3-benzothiazol-2-ylamino)-4-methyl-6,7-dihydro-5H-pyrido[2,3-c]pyridazin-8-yl]-3-[1-[[3-[2-[13-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]tridecyl-methyl-amino]ethoxy]-5,7-dimethyl-1-adamantyl]methyl]-5-methyl-pyrazol-4-yl]pyridine-2-carboxylic acid

Using General procedure for production of VHL ligand-based degraders via alkylation starting from the product of Step D as the appropriate alkylating agent and 50 mg (0.06 mmol) of Preparation 4 as the appropriate amine and hydrolysis by a treatment with TFA (125 eq) in DCM (2 mL), 36 mg of the desired product were obtained. HRMS-ESI (m/z): [M+2H]²⁺ calcd for C₇₉H₁₀₇N₁₃O₈S₂: 715.8976. found: 715.8976.

### Example 112: 6-[[6-(1,3-benzothiazol-2-ylamino)-5-methyl-pyridazin-3-yl]-[4-[[12-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-12-oxo-dodecanoyl]-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

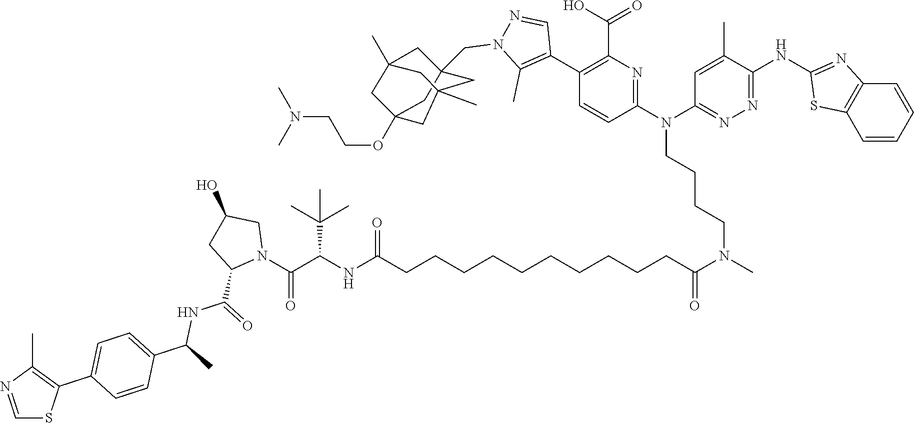 [SMILES: Cc1cc(N(CCCCN(C)C(=O)CCCCCCCCCCC(=O)N[C@H](C(=O)N2C[C@H](O)C[C@H]2C(=O)N[C@@H](C)c2ccc(-c3scnc3C)cc2)C(C)(C)C)c2ccc(-c3cnn(CC45CC6(C)CC(C)(C4)CC(OCCN(C)C)(C6)C5)c3C)c(C(=O)O)n2)nnc1Nc1nc2ccccc2s1]

### Step A: 12-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-12-oxo-dodecanoic acid

Using General procedure for the acylation and deprotection of VHL ligands starting from (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.26 mmol) and 12-tert-butoxy-12-oxo-dodecanoic acid as the appropriate acid, 134 mg of the desired product were obtained. ¹H NMR (500 MHz, dmso-d6) δ ppm 12.08 (brs, 1H), 8.99 (s, 1H), 8.37 (d, 1H), 7.78 (d, 1H), 7.44 (m, 2H), 7.38 (m, 2H), 4.92 (m, 1H), 4.51 (d, 1H), 4.42 (t, 1H), 4.28 (m, 1H), 3.61/3.59 (dd+dd, 2H), 2.46 (s, 3H), 2.24/2.11 (m+m, 2H), 2.18 (t, 2H), 2.01/1.79 (m+m, 2H), 1.51/1.46 (m+m, 2H), 1.47 (m, 2H), 1.38 (d, 3H), 1.32-1.17 (m, 12H), 0.93 (s, 9H); HRMS-ESI (m/z): [M+H]⁺ calcd for C₃₅H₅₂N₄O₆S: 657.3680. found: 657.3676.

### Step B: 6-[[6-(1,3-benzothiazol-2-ylamino)-5-methyl-pyridazin-3-yl]-[4-[[12-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-12-oxo-dodecanoyl]-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

Using Degrader Synthesis by Amide Coupling and Hydrolysis General Procedure starting from the product of Step A as the appropriate acid and 40 mg (0.04 mmol) of Preparation 19 as the appropriate amine, 15 mg of the desired product were obtained. HRMS-ESI (m/z): [M+2H]²⁺ calcd for C₇₉H₁₀₈N₁₄O₈S₂: 723.4030. found 723.4030.

### Example 113: 6-[3-(1,3-benzothiazol-2-ylamino)-4-methyl-6,7-dihydro-5H-pyrido[2,3-c]pyridazin-8-yl]-3-[1-[[3-[2-[2-[2-[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]ethoxy]ethyl-methyl-amino]ethoxy]-5,7-dimethyl-1-adamantyl]methyl]-5-methyl-pyrazol-4-yl]pyridine-2-carboxylic acid

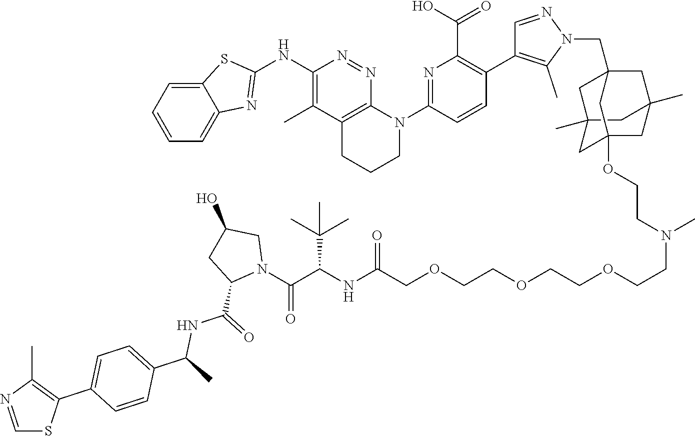 [SMILES: Cc1ncsc1-c1ccc([C@H](C)NC(=O)[C@@H]2C[C@@H](O)CN2C(=O)[C@@H](NC(=O)COCCOCCOCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1]

### Step A: tert-butyl 2-[2-[2-[2-(p-tolylsulfonyloxy)ethoxy]ethoxy]ethoxy]acetate

Using General procedure for the tosylation of the hydroxyalkyl VHL ligand-derivatives starting from 1.5 g (5.67 mmol) of tert-butyl 2-[2-[2-(2-hydroxyethoxy)ethoxy]ethoxy]acetate, 1.975 g of the desired product was obtained. ¹H NMR (500 MHz, dmso-d6) δ ppm 7.78 (d, 2H), 7.48 (d, 2H), 4.11 (t, 2H), 3.97 (s, 2H), 3.57 (t, 2H), 3.54 (m, 2H), 3.49 (m, 2H), 3.45 (m, 2H), 3.45 (m, 2H), 2.42 (s, 3H), 1.41 (s, 9H); ¹³C NMR (125 MHz, dmso-d6) δ ppm 169.8, 145.4, 132.9, 130.6, 128.1, 81.1, 70.4, 70.3, 70.2, 70.1, 70.1, 68.5, 68.3, 28.2, 21.6; HRMS-ESI [M+H]⁺ calcd for C₁₉H₃₀O₈S 436.2000. found: 436.2002.

### Step B: 2-[2-[2-[2-(p-tolylsulfonyloxy)ethoxy]ethoxy]ethoxy]acetic acid

To the product of Step A (500 mg, 1.12 mmol) in 6 mL of dichloromethane was added TFA (0.68 mL, 7.5 eq). Then, the reaction mixture was stirred for 18 h. 430 mg of the product was isolated after removing the volatiles under reduced pressure. ¹H NMR (500 MHz, dmso-d6) δ ppm 10.55 (brs, 1H), 7.78 (d, 2H), 7.48 (d, 2H), 4.11 (t, 2H), 4.00 (s, 2H), 3.57 (t, 2H), 3.56 (t, 2H), 3.49 (t, 2H), 3.45 (m, 4H), 2.42 (s, 3H); ¹³C NMR (125 MHz, dmso-d6) δ ppm 172.1, 145.4, 132.9, 130.6, 128.1, 70.4, 70.3, 70.2/70.1, 70.2, 68.3, 68.0, 21.5; HRMS-ESI (m/z): [M+H]⁺ calcd for C₁₅H₂₂O₈S: 363.1109. found 363.1109.

### Step C: 2-[2-[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]ethoxy]ethyl 4-methylbenzenesulfonate

Using General procedure for the acylation and deprotection of VHL ligands without the hydrolysis step starting from 250 mg of (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.52 mmol) and the product of Step B as the appropriate acid, 134 mg of the desired product were obtained. ¹H NMR (500 MHz, dmso-d6) δ ppm 8.98 (s, 1H), 8.44 (d, 1H), 7.79 (dm, 2H), 7.48 (dm, 2H), 7.43 (dm, 2H), 7.38 (d, 1H), 7.37 (dm, 2H), 4.90 (m, 1H), 4.54 (d, 1H), 4.44 (t, 1H), 4.28 (br., 1H), 4.11 (m, 2H), 3.95 (s, 2H), 3.63-3.45 (m, 10H), 3.58 (m, 2H), 2.45 (s, 3H), 2.42 (s, 3H), 2.05/1.77 (m+m, 2H), 1.36 (d, 3H), 0.93 (s, 9H); ¹³C NMR (125 MHz, dmso-d6) δ ppm 152.0, 130.6, 129.3, 128.1, 126.8, 70.5, 70.1, 69.2, 59.0, 57.0, 56.2, 48.2, 38.2, 26.9, 23.0, 21.6, 16.5; HRMS-ESI (m/z): [M+H]⁺ calcd for C₃₅H₅₂N₄O₁₀S₂: 789.3198. found: 789.3199.

### Step D: 6-[3-(1,3-benzothiazol-2-ylamino)-4-methyl-6,7-dihydro-5H-pyrido[2,3-c]pyridazin-8-yl]-3-[1-[[3-[2-[2-[2-[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]ethoxy]ethyl-methyl-amino]ethoxy]-5,7-dimethyl-1-adamantyl]methyl]-5-methyl-pyrazol-4-yl]pyridine-2-carboxylic acid

Using General procedure for production of VHL ligand-based degraders via alkylation starting from the product of Step C as the appropriate alkylating agent and 50 mg (0.06 mmol) of Preparation 4 as the appropriate amine and the hydrolysis by a treatment with TFA (125 eq) in DCM (2 mL), 15 mg of the desired product were obtained. HRMS-ESI (m/z): [M+H]⁺ calcd for C₇₂H₉₃N₁₃O₁₀S₂: 1364.6682. found: 1364.6682.

### Example 114: 6-[[6-(1,3-benzothiazol-2-ylamino)-5-methyl-pyridazin-3-yl]-[4-[[1-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-11-oxo-undecanoyl]-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

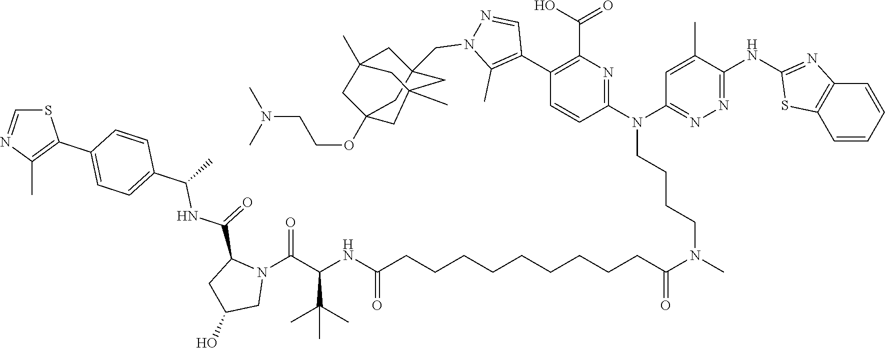 [SMILES: Cc1cc(N(CCCCN(C)C(=O)CCCCCCCCCC(=O)N[C@H](C(=O)N2C[C@H](O)C[C@H]2C(=O)N[C@@H](C)c2ccc(-c3scnc3C)cc2)C(C)(C)C)c2ccc(-c3cnn(CC45CC6(C)CC(C)(C4)CC(OCCN(C)C)(C6)C5)c3C)c(C(=O)O)n2)nnc1Nc1nc2ccccc2s1]

### Step A: 11-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-11-oxo-undecanoic acid

Using General procedure for the acylation and deprotection of VHL ligands starting from (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.62 mmol) and 11-tert-butoxy-11-oxo-undecanoic acid as the appropriate acid, 152 mg of the desired product were obtained. HPLC-MS (m/z): [M+H]⁺ calcd for C₃₄H₅₁N₄O₆S: 643. found: 643.

### Step B: 6-[[6-(1,3-benzothiazol-2-ylamino)-5-methyl-pyridazin-3-yl]-[4-[[11-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-11-oxo-undecanoyl]-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

Using Degrader Synthesis by Amide Coupling and Hydrolysis General Procedure starting from the product of Step A as the appropriate acid and 75 mg (0.08 mmol) of Preparation 19 as the appropriate amine, 22 mg of the desired product were obtained. HRMS-ESI (m/z): [M+2H]²⁺ calcd for C₇₈H₁₀₆N₁₄O₈S₂: 716.3952. found 716.3955.

### Example 115: 6-[3-(1,3-benzothiazol-2-ylamino)-4-methyl-6,7-dihydro-5H-pyrido[2,3-c]pyridazin-8-yl]-3-[1-[[3-[2-[13-[2-(2,6-dioxo-3-piperidyl)-1,3-dioxo-isoindolin-5-yl]oxytridecyl-methyl-amino]ethoxy]-5,7-dimethyl-1-adamantyl]methyl]-5-methyl-pyrazol-4-yl]pyridine-2-carboxylic acid

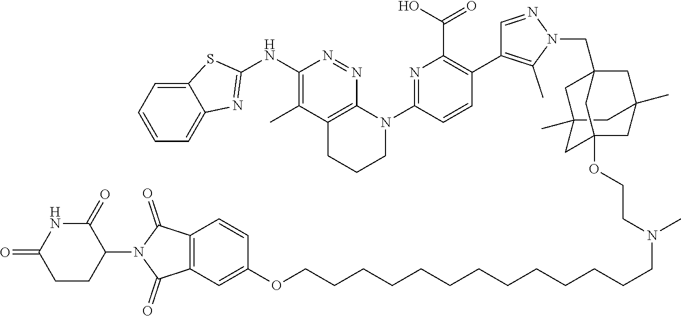 [SMILES: Cc1c(Nc2nc3ccccc3s2)nnc2c1CCCN2c1ccc(-c2cnn(CC34CC5(C)CC(C)(C3)CC(OCCN(C)CCCCCCCCCCCCCOc3ccc6c(c3)C(=O)N(C3CCC(=O)NC3=O)C6=O)(C5)C4)c2C)c(C(=O)O)n1]

### Step A: 5-(13-bromotridecoxy)-2-(2,6-dioxo-3-piperidyl)isoindoline-1,3-dione

Using General procedure for the alkylation of the 5-hydroxy thalidomide starting from 150 mg of 2-(2,6-dioxo-3-piperidyl)-5-hydroxy-isoindoline-1,3-dione (0.54 mmol) and 1,13-dibromotridecane as the appropriate dibromide, 82 mg of the desired product were obtained. ¹H NMR (500 MHz, dmso-d6) δ ppm 8.98 (s, 1H), 8.36 (d, 1H), 7.78 (d, 1H), 7.43 (d, 2H), 7.38 (d, 2H), 5.09 (brs, 1H), 4.92 (qn, 1H), 4.52 (d, 1H), 4.42 (t, 1H), 4.28 (brm, 1H), 3.61/3.58 (dd+dd, 2H), 2.45 (s, 3H), 2.25/2.09 (m+m, 2H), 2.16 (t, 2H), 2.00/1.79 (m+m, 2H), 1.51-1.21 (m, 14H), 1.39 (s, 9H), 1.37 (d, 3H), 0.93 (s, 9H); ¹³C NMR (125 MHz, dmso-d6) δ ppm 151.9, 129.3, 126.8, 69.3, 59.0, 56.8, 56.7, 48.2, 38.2, 35.3, 35.2, 28.3, 26.9, 22.9, 16.5; HRMS-ESI (m/z): [M+H]⁺ calcd for C₂₆H₃₅BrN₂O₅: 535.1802. found 535.1804.

### Step B: 6-[3-(1,3-benzothiazol-2-ylamino)-4-methyl-6,7-dihydro-5H-pyrido[2,3-c]pyridazin-8-yl]-3-[1-[[3-[2-[13-[2-(2,6-dioxo-3-piperidyl)-1,3-dioxo-isoindolin-5-yl]oxytridecyl-methyl-amino]ethoxy]-5,7-dimethyl-1-adamantyl]methyl]-5-methyl-pyrazol-4-yl]pyridine-2-carboxylic acid

Using Degrader Synthesis by Alkylation and Hydrolysis General Procedure starting from the product of Step A as the appropriate bromide and 35 mg (0.04 mmol) of Preparation 4 as the appropriate amine, 11 mg of the desired product were obtained. HRMS-ESI (m/z): [M+H]⁺ calcd for C₆₇H₈₃N₁₁O₈S: 1202.6220. found: 1202.6221.

### Example 116: 6-[[6-(1,3-benzothiazol-2-ylamino)-5-methyl-pyridazin-3-yl]-[4-[[14-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-14-oxo-tetradecanoyl]-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

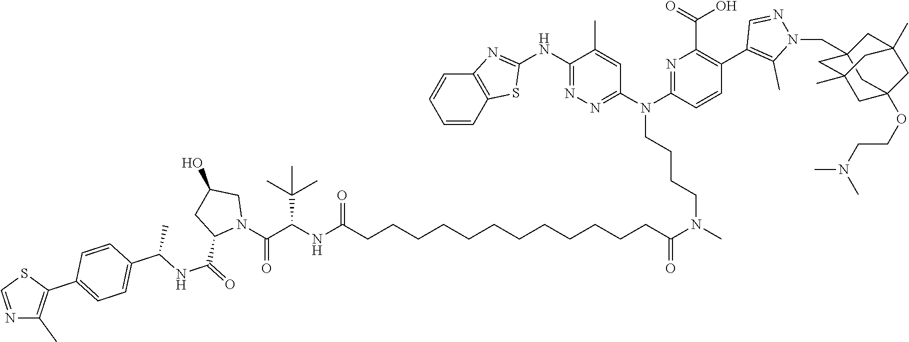 [SMILES: Cc1cc(N(CCCCN(C)C(=O)CCCCCCCCCCCCC(=O)N[C@H](C(=O)N2C[C@H](O)C[C@H]2C(=O)N[C@@H](C)c2ccc(-c3scnc3C)cc2)C(C)(C)C)c2ccc(-c3cnn(CC45CC6(C)CC(C)(C4)CC(OCCN(C)C)(C6)C5)c3C)c(C(=O)O)n2)nnc1Nc1nc2ccccc2s1]

### Step A: 14-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-14-oxo-tetradecanoic acid

Using General procedure for the acylation and deprotection of VHL ligands starting from 300 mg (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.62 mmol) and 14-tert-butoxy-14-oxo-tetradecanoic acid as the appropriate acid, 455 mg of the desired product were obtained. HPLC-MS (m/z): [M+H]⁺ calcd for C₃₇H₅₆N₄O₆S: 685. found: 685.

### Step B: 6-[[6-(1,3-benzothiazol-2-ylamino)-5-methyl-pyridazin-3-yl]-[4-[[14-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-14-oxo-tetradecanoyl]-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

Using Degrader Synthesis by Amide Coupling and Hydrolysis General Procedure starting from the product of Step A as the appropriate acid and 50 mg (0.05 mmol) of Preparation 19 as the appropriate amine, 15 mg of the desired product were obtained. HRMS-ESI (m/z): [M+2H]²⁺ calcd for C₈₁H₁₁₂N₁₄O₈S₂: 737.4187. found 737.4186.

### Example 117: 6-[3-(1,3-benzothiazol-2-ylamino)-4-methyl-6,7-dihydro-5H-pyrido[2,3-c]pyridazin-8-yl]-3-[1-[[3-[2-[11-[(2R)-2-[(1-fluorocyclopropanecarbonyl)amino]-3-[(2S,4R)-4-hydroxy-2-[[4-(4-methylthiazol-5-yl)phenyl]methylcarbamoyl]pyrrolidin-1-yl]-1,1-dimethyl-3-oxo-propyl]sulfanylundecyl-methyl-amino]ethoxy]-5,7-dimethyl-1-adamantyl]methyl]-5-methyl-pyrazol-4-yl]pyridine-2-carboxylic acid

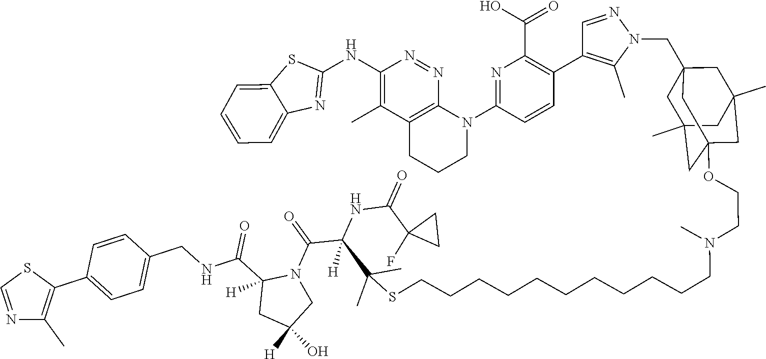 [SMILES: Cc1ncsc1-c1ccc(CNC(=O)[C@@H]2C[C@@H](O)CN2C(=O)[C@@H](NC(=O)C2(F)CC2)C(C)(C)SCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)cc1]

### Step A: (2S,4R)-1-[(2R)-3-(11-bromoundecylsulfanyl)-2-[(1-fluorocyclopropanecarbonyl)amino]-3-methyl-butanoyl]-4-hydroxy-N-[[4-(4-methylthiazol-5-yl)phenyl]methyl]pyrrolidine-2-carboxamide

Using General procedure for the alkylation of VHL ligand on thiol group starting from 75 mg of (2S,4R)-1-[(2R)-2-[(1-fluorocyclopropanecarbonyl)amino]-3-methyl-3-sulfanyl-butanoyl]-4-hydroxy-N-[[4-(4-methylthiazol-5-yl)phenyl]methyl]pyrrolidine-2-carboxamide (0.14 mmol) and 1,11-dibromoundecane as the appropriate dibromide, 97.4 mg of the desired product were obtained. ¹H NMR (500 MHz, dmso-d6) δ ppm 8.98 (s, 1H), 8.58 (t, 1H), 7.48 (d, 1H), 7.42 (d, 2H), 7.39 (d, 2H), 5.19 (brd, 1H), 4.78 (d, 1H), 4.46 (t, 1H), 4.43/4.24 (dd+dd, 2H), 4.36 (brm, 1H), 3.72/3.64 (dd+dd, 2H), 3.50 (t, 2H), 2.51 (t, 2H), 2.45 (s, 3H), 2.08/1.91 (m+m, 2H), 1.76 (qn, 2H), 1.39-1.15 (m, 20H), 1.38/1.35 (s+s, 6H); ¹³C NMR (125 MHz, dmso-d6) δ ppm 151.9, 129.2, 127.9, 69.3, 59.5, 57.1, 55.3, 42.1, 38.4, 35.7, 32.7, 28.2, 27.2/24.7, 16.5; HRMS-ESI (m/z): [M+H]⁺ calcd for C₃₆H₅₂BrFN₄O₄S₂: 767.2670. found 767.2675.

### Step B: 6-[3-(1,3-benzothiazol-2-ylamino)-4-methyl-6,7-dihydro-5H-pyrido[2,3-c]pyridazin-8-yl]-3-[1-[[3-[2-[11-[(2R)-2-[(1-fluorocyclopropanecarbonyl)amino]-3-[(2S,4R)-4-hydroxy-2-[[4-(4-methylthiazol-5-yl)phenyl]methylcarbamoyl]pyrrolidin-1-yl]-1,1-dimethyl-3-oxo-propyl]sulfanylundecyl-methyl-amino]ethoxy]-5,7-dimethyl-1-adamantyl]methyl]-5-methyl-pyrazol-4-yl]pyridine-2-carboxylic acid

Using Degrader Synthesis by Alkylation and Hydrolysis General Procedure starting from the product of Step A as the appropriate bromide and 40 mg (0.05 mmol) of Preparation 4 as the appropriate amine, 16 mg of the desired product were obtained. HRMS-ESI (m/z): [M+2H]²⁺ calcd for C₇₇H₁₀₀FN₁₃O₇S₃: 717.8580. found 717.8581.

### Example 118: 6-[3-(1,3-benzothiazol-2-ylamino)-4-methyl-6,7-dihydro-5H-pyrido[2,3-c]pyridazin-8-yl]-3-[1-[[3-[2-[13-[(2R)-2-[(1-fluorocyclopropanecarbonyl)amino]-3-[(2S,4R)-4-hydroxy-2-[[4-(4-methylthiazol-5-yl)phenyl]methylcarbamoyl]pyrrolidin-1-yl]-1,1-dimethyl-3-oxo-propyl]sulfanyltridecyl-methyl-amino]ethoxy]-5,7-dimethyl-1-adamantyl]methyl]-5-methyl-pyrazol-4-yl]pyridine-2-carboxylic acid

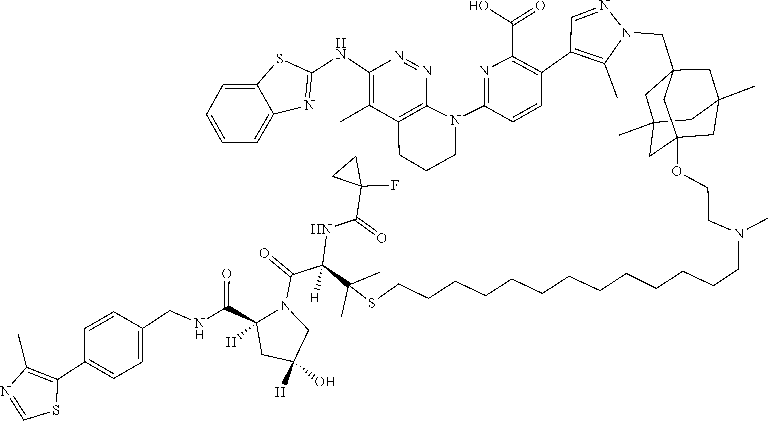 [SMILES: Cc1ncsc1-c1ccc(CNC(=O)[C@@H]2C[C@@H](O)CN2C(=O)[C@@H](NC(=O)C2(F)CC2)C(C)(C)SCCCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)cc1]

### Step A: (2S,4R)-1-[(2R)-3-(13-bromotridecylsulfanyl)-2-[(1-fluorocyclopropanecarbonyl)amino]-3-methyl-butanoyl]-4-hydroxy-N-[[4-(4-methylthiazol-5-yl)phenyl]methyl]pyrrolidine-2-carboxamide

Using General procedure for the alkylation of VHL ligand on thiol group starting from 75 mg of (2S,4R)-1-[(2R)-2-[(1-fluorocyclopropanecarbonyl)amino]-3-methyl-3-sulfanyl-butanoyl]-4-hydroxy-N-[[4-(4-methylthiazol-5-yl)phenyl]methyl]pyrrolidine-2-carboxamide (0.14 mmol) and 1,13-dibromotridecane as the appropriate dibromide, 88 mg of the desired product were obtained. ¹H NMR (500 MHz, dmso-d6) δ ppm 8.98 (s, 1H), 8.58 (t, 1H), 7.48 (d, 1H), 7.42 (d, 2H), 7.39 (d, 2H), 5.19 (brd, 1H), 4.78 (d, 1H), 4.46 (t, 1H), 4.43/4.24 (dd+dd, 2H), 4.36 (brm, 1H), 3.72/3.64 (dd+dd, 2H), 3.51 (t, 2H), 2.51 (t, 2H), 2.45 (s, 3H), 2.08/1.91 (m+m, 2H), 1.77 (qn, 2H), 1.38/1.35 (s+s, 6H), 1.38-1.15 (m, 24H); ¹³C NMR (125 MHz, dmso-d6) δ ppm 151.9, 129.2, 127.9, 69.3, 59.5, 57.1, 55.3, 42.1, 38.4, 35.7, 32.7, 28.2, 27.2/24.7, 16.5; HRMS-ESI (m/z): [M+H]⁺ calcd for C₃₈H₅₆BrFN₄O₄S₂₂: 795.2983. found 795.2987.

### Step B: 6-[3-(1,3-benzothiazol-2-ylamino)-4-methyl-6,7-dihydro-5H-pyrido[2,3-c]pyridazin-8-yl]-3-[1-[[3-[2-[13-[(2R)-2-[(1-fluorocyclopropanecarbonyl)amino]-3-[(2S,4R)-4-hydroxy-2-[[4-(4-methylthiazol-5-yl)phenyl]methylcarbamoyl]pyrrolidin-1-yl]-1,1-dimethyl-3-oxo-propyl]sulfanyltridecyl-methyl-amino]ethoxy]-5,7-dimethyl-1-adamantyl]methyl]-5-methyl-pyrazol-4-yl]pyridine-2-carboxylic acid

Using Degrader Synthesis by Alkylation and Hydrolysis General Procedure starting from the product of Step A as the appropriate bromide and 40 mg (0.05 mmol) of Preparation 4 as the appropriate amine, 18 mg of the desired product were obtained. HRMS-ESI (m/z): [M+2H]²⁺ calcd for C₇₉H₁₀₄FN₁₃O₇S₃: 731.8739. found 731.8739.

### Example 119: 6-[3-(1,3-benzothiazol-2-ylamino)-4-methyl-6,7-dihydro-5H-pyrido[2,3-c]pyridazin-8-yl]-3-[1-[[3-[2-[15-[2-(2,6-dioxo-3-piperidyl)-1,3-dioxo-isoindolin-5-yl]oxypentadecyl-methyl-amino]ethoxy]-5,7-dimethyl-1-adamantyl]methyl]-5-methyl-pyrazol-4-yl]pyridine-2-carboxylic acid

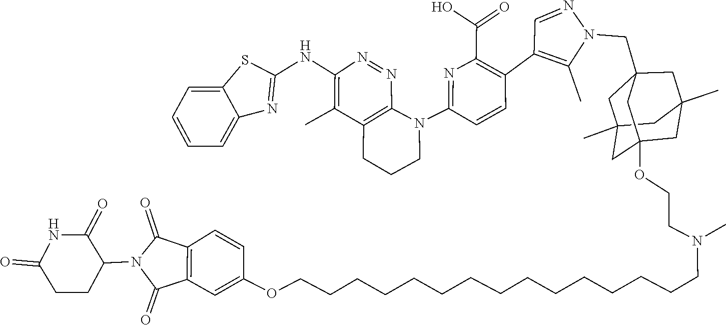 [SMILES: Cc1c(Nc2nc3ccccc3s2)nnc2c1CCCN2c1ccc(-c2cnn(CC34CC5(C)CC(C)(C3)CC(OCCN(C)CCCCCCCCCCCCCCCOc3ccc6c(c3)C(=O)N(C3CCC(=O)NC3=O)C6=O)(C5)C4)c2C)c(C(=O)O)n1]

### Step A: 5-(15-bromopentadecoxy)-2-(2,6-dioxo-3-piperidyl)isoindoline-1,3-dione

Using General procedure for the alkylation of the 5-hydroxy thalidomide starting from 200 mg of 2-(2,6-dioxo-3-piperidyl)-5-hydroxy-isoindoline-1,3-dione (0.7 mmol) and 1,15-dibromopentadecane as the appropriate dibromide, 350 mg of the desired product were obtained. ¹H NMR (500 MHz, dmso-d6) δ ppm 8.98 (s, 1H), 8.58 (t, 1H), 7.48 (d, 1H), 7.42 (d, 2H), 7.39 (d, 2H), 5.19 (brd, 1H), 4.78 (d, 1H), 4.46 (t, 1H), 4.43/4.24 (dd+dd, 2H), 4.36 (brm, 1H), 3.72/3.64 (dd+dd, 2H), 3.51 (t, 2H), 2.51 (t, 2H), 2.45 (s, 3H), 2.08/1.91 (m+m, 2H), 1.77 (qn, 2H), 1.38/1.35 (s+s, 6H), 1.38-1.15 (m, 24H); ¹³C NMR (125 MHz, dmso-d6) δ ppm 151.9, 129.2, 127.9, 69.3, 59.5, 57.1, 55.3, 42.1, 38.4, 35.7, 32.7, 28.2, 27.2/24.7, 16.5.; HRMS-ESI (m/z): [M+H]⁺ calcd for C₂₈H₃₉BrN₂O₅: 563.2115. found 563.2118.

### Step B: 6-[3-(1,3-benzothiazol-2-ylamino)-4-methyl-6,7-dihydro-5H-pyrido[2,3-c]pyridazin-8-yl]-3-[1-[[3-[2-[15-[2-(2,6-dioxo-3-piperidyl)-1,3-dioxo-isoindolin-5-yl]oxypentadecyl-methyl-amino]ethoxy]-5,7-dimethyl-1-adamantyl]methyl]-5-methyl-pyrazol-4-yl]pyridine-2-carboxylic acid

Using Degrader Synthesis by Alkylation and Hydrolysis General Procedure starting from the product of Step A as the appropriate bromide and 40 mg (0.05 mmol) of Preparation 4 as the appropriate amine, 15 mg of the desired product were obtained. HRMS-ESI (m/z): [M+H]⁺ calcd for C₆₉H₈₇N₁₁O₈S: 1230.6532. found: 1230.6531.

### Example 120: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-N-[7-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-7-oxo-heptyl]pyridine-2-carboxamide

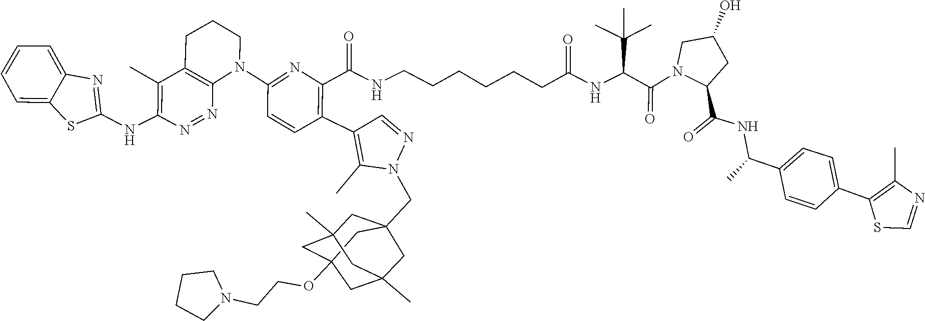 [SMILES: Cc1ncsc1-c1ccc([C@H](C)NC(=O)[C@@H]2C[C@@H](O)CN2C(=O)[C@@H](NC(=O)CCCCCCNC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C(C)(C)C)cc1]

### Step A: (2S,4R)-1-[(2S)-2-(7-aminoheptanoylamino)-3,3-dimethyl-butanoyl]-4-hydroxy-N-[(1S)-1-[4-(4-methylthiazol-5-yl)phenyl]ethyl]pyrrolidine-2-carboxamide

Using General procedure for the acylation and deprotection of VHL ligands starting from 100 mg of (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.21 mmol) and 7-(tert-butoxycarbonylamino)heptanoic acid as the appropriate acid, 92 mg of the desired product were obtained. ¹H NMR (500 MHz, dmso-d6) δ ppm 8.99 (s, 1H), 8.37 (d, 1H), 7.80 (d, 1H), 7.60 (br., 3H), 7.44 (dm, 2H), 7.38 (dm, 2H), 4.91 (m, 1H), 4.52 (d, 1H), 4.41 (t, 1H), 4.28 (br., 1H), 3.62/3.58 (dd+d, 2H), 2.76 (m, 2H), 2.45 (s, 3H), 2.26/2.11 (m+m, 2H), 2.01/1.79 (m+m, 2H), 1.57-1.19 (m, 8H), 1.37 (d, 3H), 0.93 (s, 9H); ¹³C NMR (125 MHz, dmso-d6) δ ppm 152.0, 129.3, 126.9, 69.2, 59.0, 56.8, 56.7, 48.2, 39.3, 38.2, 35.2, 26.9, 22.9, 16.4; HRMS-ESI (m/z): [M+H]⁺ calcd for C₃₀H₄₅N₅O₄S: 572.3265. found 572.3265.

### Step B: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-N-[7-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-7-oxo-heptyl]pyridine-2-carboxamide

Using Degrader Synthesis by Amide Coupling General Procedure starting from the product of Step A as the appropriate amine and 30 mg (0.03 mmol) of Preparation 21 as the appropriate acid, 15 mg of the desired product were obtained. HRMS-ESI (m/z): [M+H]⁺ calcd for C₇₄H₉₆N₁₄O₆S₂: 1341.7151. found 1341.7148.

### Example 121: 6-[[6-(1,3-benzothiazol-2-ylamino)-5-methyl-pyridazin-3-yl]-[4-[[13-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-13-oxo-tridecanoyl]-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

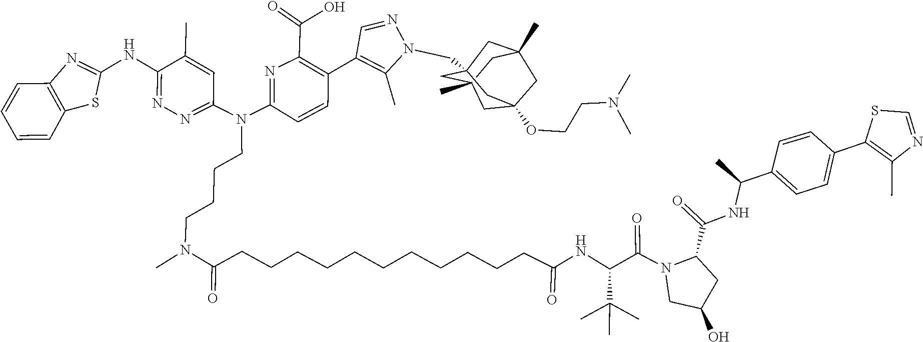 [SMILES: Cc1cc(N(CCCCN(C)C(=O)CCCCCCCCCCCC(=O)N[C@H](C(=O)N2C[C@H](O)C[C@H]2C(=O)N[C@@H](C)c2ccc(-c3scnc3C)cc2)C(C)(C)C)c2ccc(-c3cnn(C[C@]45C[C@]6(C)C[C@](C)(C4)C[C@@](OCCN(C)C)(C6)C5)c3C)c(C(=O)O)n2)nnc1Nc1nc2ccccc2s1]

### Step A: benzyl 13-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-13-oxo-tridecanoate

Using General procedure for the acylation and deprotection of VHL ligands without the hydrolysis step starting from (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.42 mmol) and 13-benzyloxy-13-oxo-tridecanoic acid as the appropriate acid, 282 mg of the desired product were obtained. ¹H NMR (500 MHz, dmso-d6) δ ppm 8.99 (s, 1H), 8.38 (d, 1H), 7.79 (d, 1H), 7.43 (m, 2H), 7.40-7.29 (m, 5H), 7.37 (m, 2H), 5.10 (d, 1H), 5.08 (s, 2H), 4.92 (m, 1H), 4.51 (d, 1H), 4.41 (t, 1H), 4.27 (m, 1H), 3.61/3.58 (m+m, 2H), 2.45 (s, 3H), 2.34 (t, 2H), 2.24/2.09 (m+m, 2H), 2.00/1.79 (m+m, 2H), 1.52 (m, 2H), 1.5/1.43 (m+m, 2H), 1.37 (d, 3H), 1.28-1.18 (m, 14H), 0.93 (s, 9H); ¹³C NMR (125 MHz, dmso-d6) δ ppm 152.0, 129.3, 126.9, 69.2, 65.7, 59.0, 56.8, 56.7, 48.1, 38.2, 35.3, 33.9, 26.9, 25.9, 25.3, 22.9, 16.5; HRMS-ESI (m/z): [M+H]⁺ calcd for C₄₃H₆₀N₄O₆S: 761.4306. found: 761.4308.

### Step B: 13-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-13-oxo-tridecanoic acid

The mixture of 89 mg (0.12 mmol) of the product of Step A in 0.6 mL of THF and 0.1 mL of water was treated with 10 eq of lithium hydroxide at 50° C. for 5 h. The product was purified by preparative reversed phase chromatography using MeCN and 25 mM aqueous TFA solution as eluents, to give 110 mg of the desired product 1H NMR (500 MHz, dmso-d6) δ ppm 8.98 (s, 1H), 8.49 (d, 1H), 7.82 (d, 1H), 7.43 (d, 2H), 7.37 (d, 2H), 4.90 (qn, 1H), 4.51 (d, 1H), 4.42 (t, 1H), 4.27 (brm, 1H), 3.60/3.57 (dd+dd, 2H), 2.45 (s, 3H), 2.24/2.10 (m+m, 2H), 2.01/1.77 (m+m, 2H), 1.91 (t, 2H), 1.52-1.19 (m, 18H), 1.37 (d, 3H), 0.93 (s, 9H); ¹³C NMR (125 MHz, dmso-d6) δ ppm 151.9, 129.3, 126.9, 69.2, 59.0, 56.9, 56.8, 48.2, 38.4, 38.2, 35.3, 26.9, 22.9, 16.5; HRMS-ESI (m/z): [M+H]⁺ calcd for C₃₆H₅₄N₄O₆S: 671.3837. found: 671.3836.

### Step C: 6-[[6-(1,3-benzothiazol-2-ylamino)-5-methyl-pyridazin-3-yl]-[4-[[13-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-13-oxo-tridecanoyl]-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

Using Degrader Synthesis by Amide Coupling and Hydrolysis General Procedure starting from the product of Step B as the appropriate acid and 50 mg (0.05 mmol) of Preparation 19 as the appropriate amine, 28 mg of the desired product were obtained. HRMS-ESI (m/z): [M+2H]²⁺ calcd for C₈₀H₁₁₀N₁₄O₈S₂: 730.4109. found 730.4109.

### Example 122: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-N-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-5-oxo-pentyl]pyridine-2-carboxamide

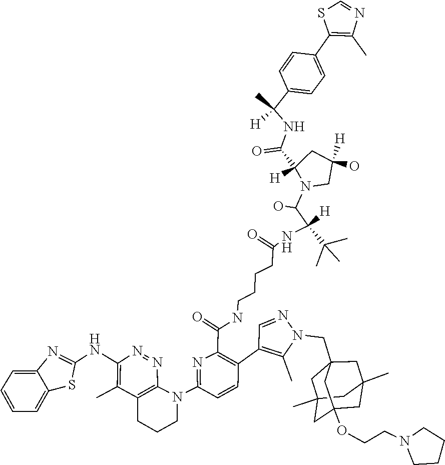

### Step A: (2S,4R)-1-[(2S)-2-(5-aminopentanoylamino)-3,3-dimethyl-butanoyl]-4-hydroxy-N-[(1S)-1-[4-(4-methylthiazol-5-yl)phenyl]ethyl]pyrrolidine-2-carboxamide

Using General procedure for the acylation and deprotection of VHL ligands starting from 150 mg of (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.31 mmol) and 5-(tert-butoxycarbonylamino)pentanoic acid as the appropriate acid, 180 mg of the desired product were obtained. HPLC-MS (m/z): [M+H]⁺ calcd for C₂₈H₄₁N₅O₄S: 544. found 544.

### Step B: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-N-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-5-oxo-pentyl]pyridine-2-carboxamide

Using Degrader Synthesis by Amide Coupling General Procedure starting from the product of Step A as the appropriate amine and 73 mg (0.07 mmol) of Preparation 21 as the appropriate acid, 15 mg of the desired product were obtained. HRMS-ESI (m/z): [M+H]⁺ calcd for C₇₂H₉₂N₁₄O₆S₂: 1313.6838. found 1313.6837.

### Example 123: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-N-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propyl]pyridine-2-carboxamide

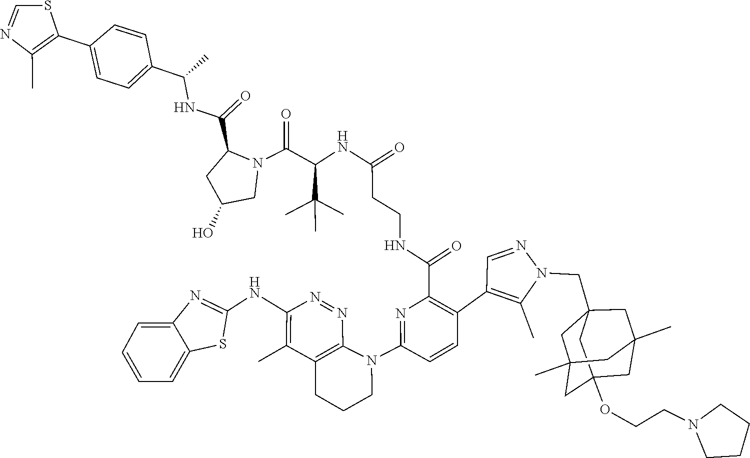 [SMILES: Cc1ncsc1-c1ccc([C@H](C)NC(=O)[C@@H]2C[C@@H](O)CN2C(=O)[C@@H](NC(=O)CCNC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C(C)(C)C)cc1]

### Step A: (2S,4R)-1-[(2S)-2-(3-aminopropanoylamino)-3,3-dimethyl-butanoyl]-4-hydroxy-N-[(1S)-1-[4-(4-methylthiazol-5-yl)phenyl]ethyl]pyrrolidine-2-carboxamide

Using General procedure for the acylation and deprotection of VHL ligands starting from 150 mg of (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.31 mmol) and 3-(tert-butoxycarbonylamino)propanoic acid as the appropriate acid, 146 mg of the desired product were obtained. ¹H NMR (500 MHz, dmso-d6) δ ppm 9.02 (s, 1H), 8.42 (d, 1H), 8.23 (d, 1H), 7.85 (br., 3H), 7.44 (dm, 2H), 7.38 (dm, 2H), 4.91 (m, 1H), 4.52 (d, 1H), 4.42 (t, 1H), 4.29 (br., 1H), 3.63/3.56 (dd+d, 2H), 2.96 (m, 2H), 2.59 (m, 2H), 2.46 (s, 3H), 2.02/1.79 (m+m, 2H), 1.37 (d, 3H), 0.95 (s, 9H); ¹³C NMR (125 MHz, dmso-d6) δ ppm 152.1, 129.3, 126.9, 69.2, 59.0, 57.1, 56.8, 48.2, 38.3, 35.7, 32.2, 26.9, 22.9, 16.4; HRMS-ESI (m/z): [M+H]⁺ calcd for C₂₆H₃₇N₅O₄S: 516.2639. found 516.2643.

### Step B: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-N-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propyl]pyridine-2-carboxamide

Using Degrader Synthesis by Amide Coupling General Procedure starting from the product of Step A as the appropriate amine and 43 mg (0.04 mmol) of Preparation 21 as the appropriate acid, 26 mg of the desired product were obtained. HRMS-ESI (m/z): [M+H]⁺ calcd for C₇₀H₈₈N₁₄O₆S₂: 1285.6525. found 1285.6533.

### Example 124: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-N-[9-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-9-oxo-nonyl]pyridine-2-carboxamide

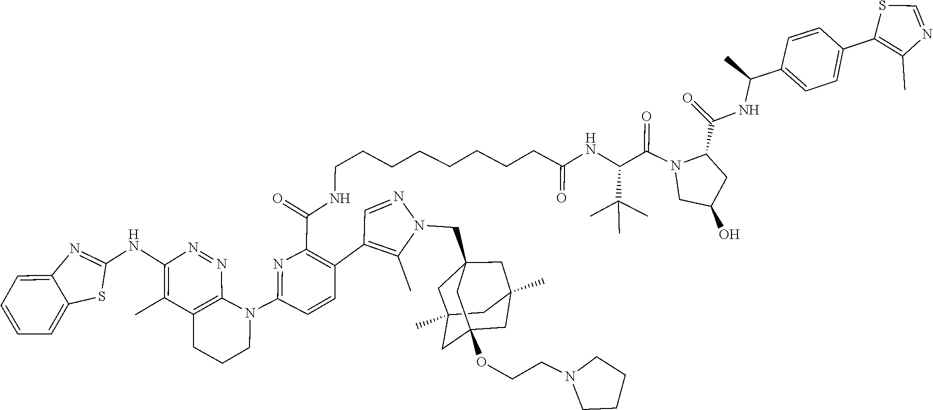 [SMILES: Cc1ncsc1-c1ccc([C@H](C)NC(=O)[C@@H]2C[C@@H](O)CN2C(=O)[C@@H](NC(=O)CCCCCCCCNC(=O)c2nc(N3CCCc4c3nnc(Nc3nc5ccccc5s3)c4C)ccc2-c2cnn(C[C@]34C[C@]5(C)C[C@](C)(C3)C[C@@](OCCN3CCCC3)(C5)C4)c2C)C(C)(C)C)cc1]

### Step A: (2S,4R)-1-[(2S)-2-(9-aminononanoylamino)-3,3-dimethyl-butanoyl]-4-hydroxy-N-[(1S)-1-[4-(4-methylthiazol-5-yl)phenyl]ethyl]pyrrolidine-2-carboxamide

Using General procedure for the acylation and deprotection of VHL ligands starting from 150 mg of (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.31 mmol) and 9-(tert-butoxycarbonylamino)nonanoic acid as the appropriate acid, 180 mg of the desired product were obtained. ¹H NMR (500 MHz, dmso-d6) δ ppm 9.02 (s, 1H), 8.40 (d, 1H), 7.84 (brs, 3H), 7.79 (d, 1H), 7.44 (d, 2H), 7.38 (d, 2H), 4.91 (qn, 1H), 4.52 (d, 1H), 4.42 (t, 1H), 4.28 (brm, 1H), 3.61/3.58 (dd+dd, 2H), 2.74 (m, 2H), 2.46 (s, 3H), 2.25/2.11 (m+m, 2H), 2.01/1.78 (m+m, 2H), 1.53 (qn, 2H), 1.49/1.45 (m+m, 2H), 1.37 (d, 3H), 1.32-1.21 (m, 8H), 0.93 (s, 9H); ¹³C NMR (125 MHz, dmso-d6) δ ppm 152.1, 129.3, 126.9, 69.3, 59.0, 56.8, 56.8, 48.2, 39.2, 38.2, 35.3, 27.4, 26.9, 25.9, 22.9, 16.4; HRMS-ESI (m/z): [M+H]⁺ calcd for C₃₂H₄₉N₅O₄S: 600.3578. found 600.3580.

### Step B: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-N-[9-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-9-oxo-nonyl]pyridine-2-carboxamide

Using Degrader Synthesis by Amide Coupling General Procedure starting from the product of Step A as the appropriate amine and 45 mg (0.05 mmol) of Preparation 21 as the appropriate acid, 15 mg of the desired product were obtained. HRMS-ESI (m/z): [M+H]⁺ calcd for C₇₆H₁₀₀N₁₄O₆S₂: 1369.7464. found 1369.7472.

### Example 125: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-N-[11-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-1-oxo-undecyl]pyridine-2-carboxamide

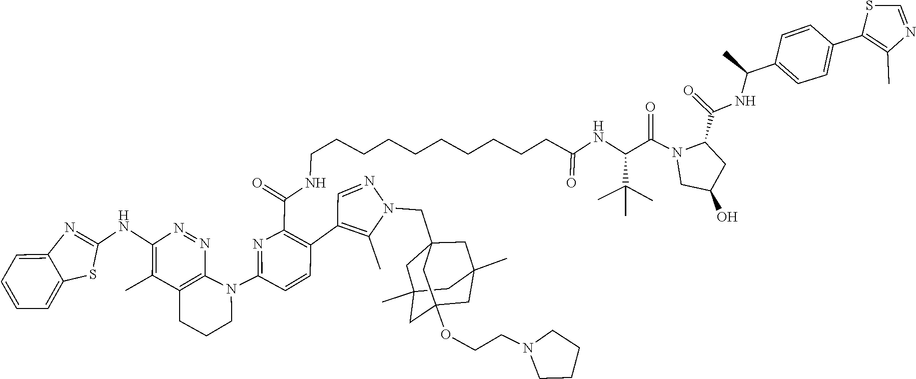 [SMILES: Cc1ncsc1-c1ccc([C@H](C)NC(=O)[C@@H]2C[C@@H](O)CN2C(=O)[C@@H](NC(=O)CCCCCCCCCCNC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C(C)(C)C)cc1]

### Step A: (2S,4R)-1-[(2S)-2-(11-aminoundecanoylamino)-3,3-dimethyl-butanoyl]-4-hydroxy-N-[(1S)-1-[4-(4-methylthiazol-5-yl)phenyl]ethyl]pyrrolidine-2-carboxamide

Using General procedure for the acylation and deprotection of VHL ligands starting from 150 mg of (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.31 mmol) and 11-(tert-butoxycarbonylamino)undecanoic acid as the appropriate acid, 117 mg of the desired product were obtained. ¹H NMR (500 MHz, dmso-d6) δ ppm 9.09 (s, 1H), 8.41 (d, 1H), 7.96 (brs, 3H), 7.79 (d, 1H), 7.44 (d, 2H), 7.39 (d, 2H), 4.91 (qn, 1H), 4.51 (d, 1H), 4.42 (t, 1H), 4.27 (brm, 1H), 3.61/3.58 (dd+dd, 2H), 2.73 (m, 2H), 2.46 (s, 3H), 2.24/2.10 (m+m, 2H), 2.02/1.78 (m+m, 2H), 1.53 (qn, 2H), 1.49/1.45 (m+m, 2H), 1.37 (d, 3H), 1.31-1.19 (m, 12H), 0.93 (s, 9H); ¹³C NMR (125 MHz, dmso-d6) δ ppm 152.4, 129.3, 126.9, 69.2, 59.0, 56.8, 56.8, 48.2, 39.2, 38.2, 35.3, 27.4, 26.9, 25.9, 22.9, 16.2 HRMS-ESI (m/z): [M+H]⁺ calcd for C₃₄H₅₃N₅O₄S: 628.3891. found 628.3894.

### Step B: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-N-[11-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-11-oxo-undecyl]pyridine-2-carboxamide

Using Degrader Synthesis by Amide Coupling General Procedure starting from the product of Step A as the appropriate amine and 43 mg (0.04 mmol) of Preparation 21 as the appropriate acid, 22 mg of the desired product were obtained. HRMS-ESI (m/z): [M+H]⁺ calcd for C₇₈H₁₀₄N₁₄O₆S₂: 1397.7777. found 1397.7783.

### Example 126: 6-[[6-(1,3-benzothiazol-2-ylamino)-5-methyl-pyridazin-3-yl]-[4-[10-[(2R)-2-[(1-fluorocyclopropanecarbonyl)amino]-3-[(2S,4R)-4-hydroxy-2-[[4-(4-methylthiazol-5-yl)phenyl]methylcarbamoyl]pyrrolidin-1-yl]-1,1-dimethyl-3-oxo-propyl]sulfanyldecanoyl-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

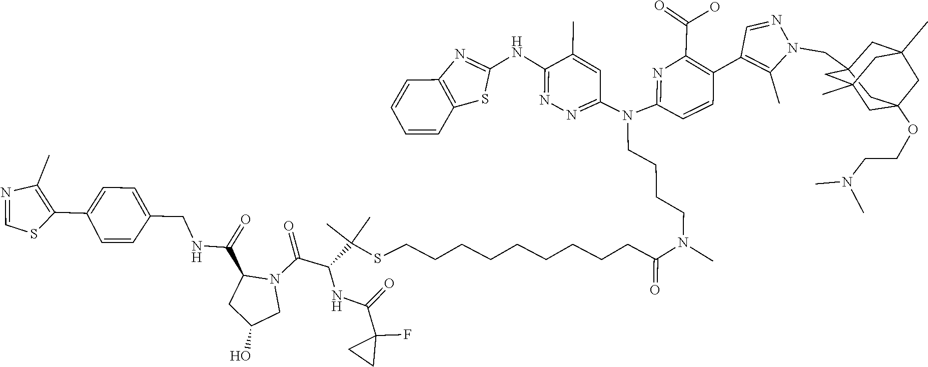

### Step A: 10-[(2R)-2-[(1-fluorocyclopropanecarbonyl)amino]-3-[(2S,4R)-4-hydroxy-2-[[4-(4-methylthiazol-5-yl)phenyl]methylcarbamoyl]pyrrolidin-1-yl]-1,1-dimethyl-3-oxo-propyl]sulfanyldecanoic acid

Using General procedure for the alkylation of VHL ligand on thiol group starting from 75 mg of (2S,4R)-1-[(2R)-2-[(1-fluorocyclopropanecarbonyl)amino]-3-methyl-3-sulfanyl-butanoyl]-4-hydroxy-N-[[4-(4-methylthiazol-5-yl)phenyl]methyl]pyrrolidine-2-carboxamide (0.14 mmol) and tert-butyl 10-bromodecanoate as the appropriate bromide, followed with the TFA deprotection method of the General procedure for the acylation and deprotection of VHL ligands afforded 77 mg of the product. HPLC-MS (m/z): [M+H]⁺ calcd for C₃₅H₅₀FN₄O₆S₂: 705. found 705.

### Step B: 6-[[6-(1,3-benzothiazol-2-ylamino)-5-methyl-pyridazin-3-yl]-[4-[10-[(2R)-2-[(1-fluorocyclopropanecarbonyl)amino]-3-[(2S,4R)-4-hydroxy-2-[[4-(4-methylthiazol-5-yl)phenyl]methylcarbamoyl]pyrrolidin-1-yl]-1,1-dimethyl-3-oxo-propyl]sulfanyldecanoyl-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

Using Degrader Synthesis by Amide Coupling and Hydrolysis General Procedure starting from the product of Step A as the appropriate acide and 60 mg (0.05 mmol) of Preparation 19 as the appropriate amine, 15 mg of the desired product were obtained. HRMS-ESI (m/z): [M+2H]²⁺ calcd for C₇₉H₁₀₅FN₁₄O₈S₃: 747.3765. found: 747.3768.

### Example 127: 6-[[6-(1,3-benzothiazol-2-ylamino)-5-methyl-pyridazin-3-yl]-[4-[13-[(2R)-2-[(1-fluorocyclopropanecarbonyl)amino]-3-[(2S,4R)-4-hydroxy-2-[[4-(4-methylthiazol-5-yl)phenyl]methylcarbamoyl]pyrrolidin-1-yl]-1,1-dimethyl-3-oxo-propyl]sulfanyltridecanoyl-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

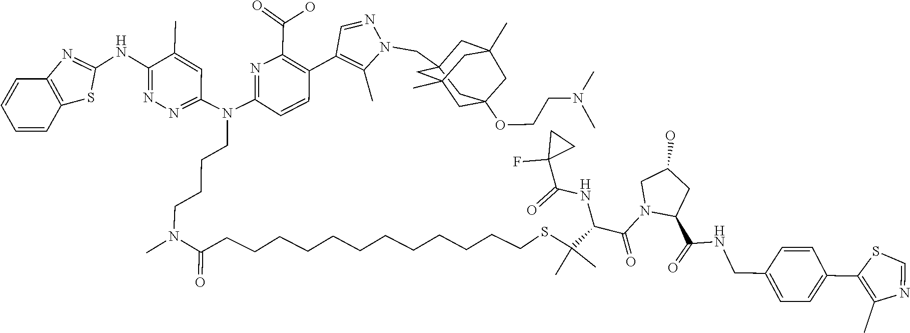

### Step A: methyl 13-[(2R)-2-[(1-fluorocyclopropanecarbonyl)amino]-3-[(2S,4R)-4-hydroxy-2-[[4-(4-methylthiazol-5-yl)phenyl]methylcarbamoyl]pyrrolidin-1-yl]-1,1-dimethyl-3-oxo-propyl]sulfanyltridecanoate

Using General procedure for the alkylation of VHL ligand on thiol group starting from 100 mg of (2S,4R)-1-[(2R)-2-[(1-fluorocyclopropanecarbonyl)amino]-3-methyl-3-sulfanyl-butanoyl]-4-hydroxy-N-[[4-(4-methylthiazol-5-yl)phenyl]methyl]pyrrolidine-2-carboxamide (0.19 mmol) and methyl 13-bromotridecanoate, to give 97 mg of the product. ¹H NMR (500 MHz, dmso-d6) δ ppm 8.99 (s, 1H), 8.58 (t, 1H), 7.47 (d, 1H), 7.41 (d, 2H), 7.38 (d, 2H), 4.78 (d, 1H), 4.46 (t, 1H), 4.42/4.23 (dd+dd, 2H), 4.36 (brm, 1H), 3.72/3.64 (dd+dd, 2H), 3.57 (s, 3H), 2.51 (t, 2H), 2.45 (s, 3H), 2.27 (t, 2H), 2.08/1.91 (m+m, 2H), 1.52-1.15 (m, 24H), 1.38/1.34 (s/s, 6H); ¹³C NMR (125 MHz, dmso-d6) δ ppm 151.9, 129.2, 127.3, 69.4, 59.5, 57.0, 55.3, 51.6, 42.1, 38.4, 33.8, 28.2, 27.2/24.7, 16.5; HRMS (ESI) [M+H]⁺ calcd for C₃₉H₅₈FN₄O₆S₂: 761.3776. found 761.3777.

### Step B: 13-[(2R)-2-[(1-fluorocyclopropanecarbonyl)amino]-3-[(2S,4R)-4-hydroxy-2-[[4-(4-methylthiazol-5-yl)phenyl]methylcarbamoyl]pyrrolidin-1-yl]-1,1-dimethyl-3-oxo-propyl]sulfanyltridecanoic acid

The mixture of 84 mg (0.11 mmol) of the product of Step A in 0.6 mL of THF and 0.1 mL of water was treated with 10 eq of lithium hydroxide at 50° C. for 5 h. The product was purified by preparative reversed phase chromatography using MeCN and 25 mM aqueous TFA solution as eluents, to give 68 mg of the desired product. ¹H NMR (500 MHz, dmso-d6) δ ppm 8.94 (s, 1H), 7.44-7.34 (m, 4H), 4.68 (brs, 1H), 4.50 (t, 1H), 4.38/4.27 (d+d, 2H), 4.30 (brs, 1H), 3.84-3.46 (brs, 2H), 2.52 (t, 2H), 2.45 (s, 3H), 2.08/1.94 (m+m, 2H), 1.86 (t, 2H), 1.50-1.04 (m, 24H), 1.36/1.32 (s, 6H); HRMS (ESI) [M+H]⁺ calcd for C₈H₅₆FN₄O₆S₂: 747.3620. found 747.3614.

### Step C: 6-[[6-(1,3-benzothiazol-2-ylamino)-5-methyl-pyridazin-3-yl]-[4-[13-[(2R)-2-[(1-fluorocyclopropanecarbonyl)amino]-3-[(2S,4R)-4-hydroxy-2-[[4-(4-methylthiazol-5-yl)phenyl]methylcarbamoyl]pyrrolidin-1-yl]-1,1-dimethyl-3-oxo-propyl]sulfanyltridecanoyl-methyl-amino]butyl]amino]-3-[1-[[3-[2-(dimethylamino)ethoxy]-5,7-dimethyl-1-adamantyl]methyl]-5-methyl-pyrazol-4-yl]pyridine-2-carboxylic acid

Using Degrader Synthesis by Amide Coupling and Hydrolysis General Procedure starting from the product of Step B as the appropriate acide and 40 mg (0.04 mmol) of Preparation 19 as the appropriate amine, 15 mg of the desired product were obtained. HRMS-ESI (m/z): [M+2H]²⁺ calcd for C₈₂H₁₁₁FN₁₄O₈S₃: 768.4000. found: 768.4005.

### Example 128: N′-[4-[[6-(1,3-benzothiazol-2-ylamino)-5-methyl-pyridazin-3-yl]-[5-[1-[[3-[2-(dimethylamino)ethoxy]-5,7-dimethyl-1-adamantyl]methyl]-5-methyl-pyrazol-4-yl]-6-(dimethylcarbamoyl)-2-pyridyl]amino]butyl]-N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]-N′-methyl-dodecanediamide

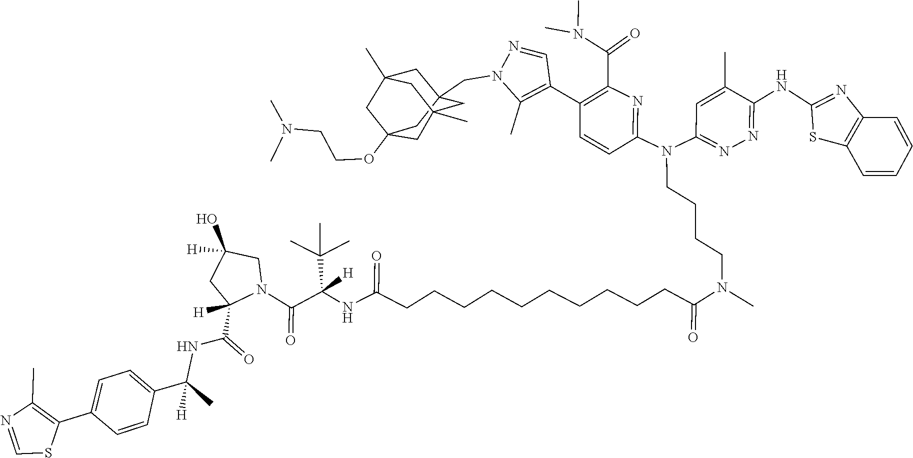 [SMILES: Cc1cc(N(CCCCN(C)C(=O)CCCCCCCCCCC(=O)N[C@H](C(=O)N2C[C@H](O)C[C@H]2C(=O)N[C@@H](C)c2ccc(-c3scnc3C)cc2)C(C)(C)C)c2ccc(-c3cnn(CC45CC6(C)CC(C)(C4)CC(OCCN(C)C)(C6)C5)c3C)c(C(=O)N(C)C)n2)nnc1Nc1nc2ccccc2s1]

To the mixture of 22 mg (0.01 mmol) of the product of Example 112 and 0.008 mL (3 eq) of N-ethyl-N-isopropyl-propan-2-amine in 1 ml of DMF were added 7.3 mg (1.5 eq) of TBTU. After 20 minutes, 0.015 mL (2 eq) of dimethylamine was also added. Then, the reaction was stirred for 2 h. The product was purified by preparative reversed phase chromatography, using MeCN and 25 mM aqueous TFA solution as eluents, to give 17 mg of the desired product. HRMS-ESI (m/z): [M+2H]²⁺ calcd for C₈₁H₁₁₃N₁₅O₇S₂: 736.9267. found 736.9266.

### Example 129: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-N-[8-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-8-oxo-octyl]pyridine-2-carboxamide

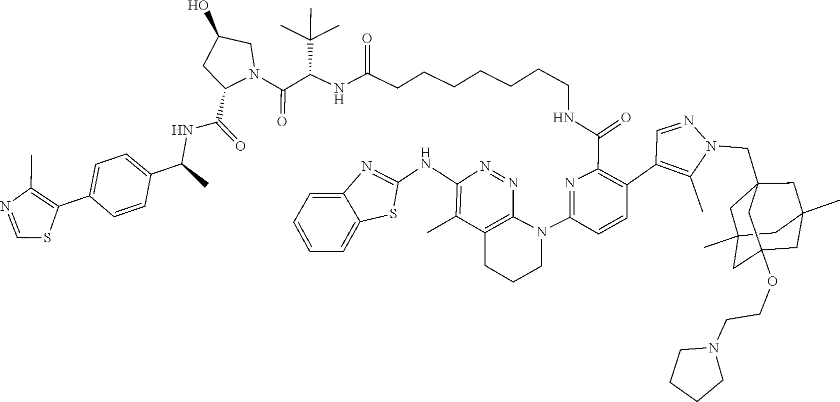 [SMILES: Cc1ncsc1-c1ccc([C@H](C)NC(=O)[C@@H]2C[C@@H](O)CN2C(=O)[C@@H](NC(=O)CCCCCCCNC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C(C)(C)C)cc1]

Step A: (2S,4R)-1-[(2S)-2-(8-aminooctanoylamino)-3,3-dimethyl-butanoyl]-4-hydroxy-N-[(1S)-1-[4-(4-methylthiazol-5-yl)phenyl]ethyl]pyrrolidine-2-carboxamide

Using General procedure for the acylation and deprotection of VHL ligands starting from 125 mg of (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.26 mmol) and 8-(tert-butoxycarbonylamino)octanoic acid as the appropriate acid, 92 mg of the desired product were obtained. ¹H NMR (500 MHz, dmso-d6) δ ppm 9.03 (s, 1H), 8.41 (d, 1H), 7.85 (brs, 3H), 7.79 (d, 1H), 7.44 (m, 2H), 7.38 (m, 2H), 4.91 (m, 1H), 4.51 (d, 1H), 4.41 (t, 1H), 4.28 (m, 1H), 3.61/3.58 (m+m, 2H), 2.75 (m, 2H), 2.46 (s, 3H), 2.25/2.11 (m+m, 2H), 2.01/1.78 (m+m, 2H), 1.52 (m, 2H), 1.50/1.46 (m+m, 2H), 1.37 (d, 3H), 1.33-1.18 (m, 6H), 0.93 (s, 9H); ¹³C NMR (125 MHz, dmso-d6) δ ppm 152.0, 129.3, 126.9, 69.2, 58.9, 56.7, 56.7, 48.1, 39.2, 38.2, 35.2, 27.4, 26.9, 25.7, 22.9, 16.4; HRMS-ESI (m/z): [M+H]⁺ calcd for C₃₁H₄₈N₅O₄S: 586.3422. found 586.3425.

### Step B: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-N-[8-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-8-oxo-octyl]pyridine-2-carboxamide

Using Degrader Synthesis by Amide Coupling General Procedure starting from the product of Step A as the appropriate amine and 48 mg (0.06 mmol) of Preparation 21 as the appropriate acid, 28 mg of the desired product were obtained. HRMS-ESI (m/z): [M+2H]²⁺ calcd for C₇₅H₉₈N₁₄O₆S₂: 678.3690. found 678.3698.

### Example 130: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-N-[10-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-10-oxo-decyl]pyridine-2-carboxamide

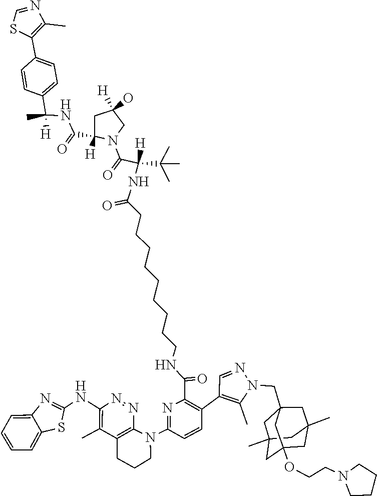

### Step A: (2S,4R)-1-[(2S)-2-(10-aminodecanoylamino)-3,3-dimethyl-butanoyl]-4-hydroxy-N-[(1S)-1-[4-(4-methylthiazol-5-yl)phenyl]ethyl]pyrrolidine-2-carboxamide

Using General procedure for the acylation and deprotection of VHL ligands starting from 125 mg of (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.26 mmol) and 10-(tert-butoxycarbonylamino)decanoic acid as the appropriate acid, 155 mg of the desired product were obtained. ¹H NMR (500 MHz, dmso-d6) δ ppm 9.02 (s, 1H), 8.66/8.39 (d, 1H), 7.81 (brs, 3H), 7.79 (d, 1H), 7.44 (m, 2H), 7.38 (m, 2H), 4.91 (m, 1H), 4.51 (d, 1H), 4.41 (t, 1H), 4.28 (m, 1H), 3.61/3.58 (m+m, 2H), 2.74 (m, 2H), 2.45 (s, 3H), 2.25/2.10 (m+m, 2H), 2.01/1.79 (m+m, 2H), 1.52 (m, 2H), 1.50/1.44 (m+m, 2H), 1.37 (d, 3H), 1.33-1.17 (m, 10H), 0.93 (s, 9H); ¹³C NMR (125 MHz, dmso-d6) δ ppm 152.0, 129.3, 126.9, 69.1, 58.9, 56.6, 56.6, 48.1, 39.2, 38.1, 35.3, 27.4, 26.9, 25.8, 22.9, 16.4; HRMS-ESI (m/z): [M+H]⁺ calcd for C₃₃H₅₁N₅O₄S: 614.3737. found 614.3734.

### Step B: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-N-[10-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-10-oxo-decyl]pyridine-2-carboxamide

Using Degrader Synthesis by Amide Coupling General Procedure starting from the product of Step A as the appropriate amine and 46 mg (0.06 mmol) of Preparation 21 as the appropriate acid, 29 mg of the desired product were obtained. HRMS-ESI (m/z): [M+2H]²⁺ calcd for C₇₇H₁₀₂N₁₄O₆S₂: 692.3846. found 692.3850.

### Example 131: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-N-[12-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-12-oxo-dodecyl]pyridine-2-carboxamide

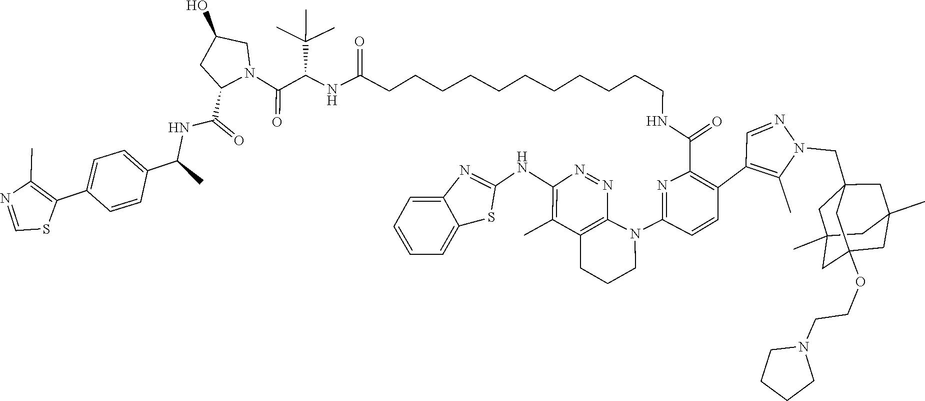 [SMILES: Cc1ncsc1-c1ccc([C@H](C)NC(=O)[C@@H]2C[C@@H](O)CN2C(=O)[C@@H](NC(=O)CCCCCCCCCCCNC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C(C)(C)C)cc1]

### Step A: (2S,4R)-1-[(2S)-2-(12-aminododecanoylamino)-3,3-dimethyl-butanoyl]-4-hydroxy-N-[(1S)-1-[4-(4-methylthiazol-5-yl)phenyl]ethyl]pyrrolidine-2-carboxamide

Using General procedure for the acylation and deprotection of VHL ligands starting from 125 mg of (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.26 mmol) and 12-(tert-butoxycarbonylamino)dodecanoic acid as the appropriate acid, 165 mg of the desired product were obtained. ¹H NMR (500 MHz, dmso-d6) δ ppm 9.01 (s, 1H), 8.66/8.39 (d, 1H), 7.86/7.79 (d, 1H), 7.80 (brs, 3H), 7.44 (m, 2H), 7.38 (m, 2H), 4.91 (m, 1H), 4.51 (d, 1H), 4.41 (t, 1H), 4.27 (m, 1H), 3.61/3.58 (m+m, 2H), 2.74 (m, 2H), 2.46 (s, 3H), 2.24/2.09 (m+m, 2H), 2.01/1.79 (m+m, 2H), 1.52 (m, 2H), 1.49/1.43 (m+m, 2H), 1.37 (d, 3H), 1.33-1.17 (m, 14H), 0.93 (s, 9H); ¹³C NMR (125 MHz, dmso-d6) δ ppm 151.9, 129.3, 126.8, 69.1, 58.9, 56.7, 56.7, 48.1, 39.2, 38.2, 35.3, 27.4, 27.0, 25.9, 22.9, 16.4; HRMS-ESI (m/z): [M+H]⁺ calcd for C₃₅H₅₅N₅O₄S: 642.4048. found 642.4046.

### Step B: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-N-[12-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-12-oxo-dodecyl]pyridine-2-carboxamide

Using Degrader Synthesis by Amide Coupling General Procedure starting from the product of Step A as the appropriate amine and 44 mg (0.05 mmol) of Preparation 21 as the appropriate acid, 21 mg of the desired product were obtained. HRMS-ESI (m/z): [M+2H]²⁺ calcd for C₇₉H₁₀₆N₁₄O₆S₂: 706.4003. found 706.4005.

### Example 132: 6-[3-(1,3-benzothiazol-2-ylamino)-4-methyl-6,7-dihydro-5H-pyrido[2,3-c]pyridazin-8-yl]-3-[1-[[3-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethyl-methyl-amino]ethoxy]-5,7-dimethyl-1-adamantyl]methyl]-5-methyl-pyrazol-4-yl]pyridine-2-carboxylic acid

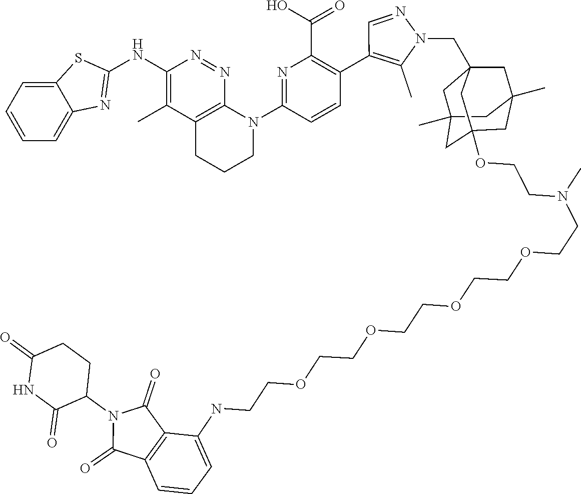 [SMILES: Cc1c(Nc2nc3ccccc3s2)nnc2c1CCCN2c1ccc(-c2cnn(CC34CC5(C)CC(C)(C3)CC(OCCN(C)CCOCCOCCOCCOCCNc3cccc6c3C(=O)N(C3CCC(=O)NC3=O)C6=O)(C5)C4)c2C)c(C(=O)O)n1]

### Step A: 2-(2,6-dioxo-3-piperidyl)-4-[2-[2-[2-[2-(2-hydroxyethoxy)ethoxy]ethoxy]ethoxy]ethylamino]isoindoline-1,3-dione

Using the General procedure for the nucleophilic substitution of fluoro-thalidomide starting from 2-(2,6-dioxo-3-piperidyl)-4-fluoro-isoindoline-1,3-dione (0.72 mmol, 200 mg), and 2-[2-[2-[2-(2-aminoethoxy)ethoxy]ethoxy]ethoxy]ethanol as the appropriate amine, 35 mg of the desired product were obtained. HRMS-ESI (m/z): [M+H]⁺ calcd for C₂₃H₃₂N₃O₉: 494.2133. found: 494.2135.

### Step B: 2-(2,6-dioxo-3-piperidyl)-4-[2-[2-[2-[2-(2-iodoethoxy)ethoxy]ethoxy]ethoxy]eth ylamino]isoindoline-1,3-dione

Using the General procedure for the iodination of hydroxyalkyl derivative of thalidomide starting from the product of Step A (35 mg), 18 mg of the desired product were obtained. HRMS-ESI (m/z): [M+H]⁺ calcd for C₂₃H₃₁IN₃O₈: 604.1150. found: 604.1150.

### Step C: 6-[3-(1,3-benzothiazol-2-ylamino)-4-methyl-6,7-dihydro-5H-pyrido[2,3-c]pyridazin-8-yl]-3-[1-[[3-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (22 mg) and the product of Step B as the appropriate alkylating agent, 16 mg of the desired product were obtained. HRMS-ESI (m/z): [M+H]⁺ calcd for C₆₄H₇₉N₁₂O₁₁: 1223.5706. found: 1223.5720.

### Example 133: 6-[3-(1,3-benzothiazol-2-ylamino)-4-methyl-6,7-dihydro-5H-pyrido[2,3-c]pyridazin-8-yl]-3-[1-[[3-[2-[[10-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-10-oxo-decyl]-methyl-amino]ethoxy]-5,7-dimethyl-1-adamantyl]methyl]-5-methyl-pyrazol-4-yl]pyridine-2-carboxylic acid

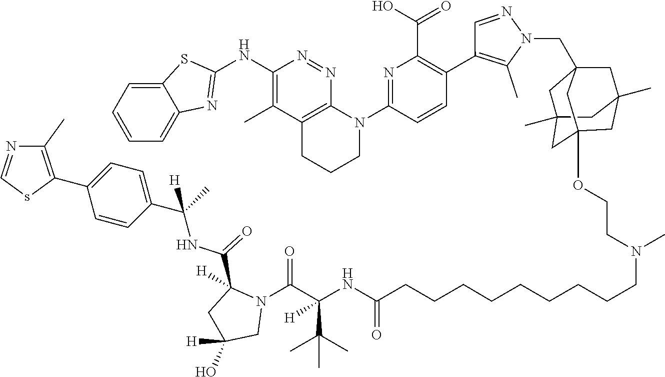

### Step A: (2S,4R)-4-hydroxy-1-[(2S)-2-(10-hydroxydecanoylamino)-3,3-dimethyl-butanoyl]-N-[(1S)-1-[4-(4-methylthiazol-5-yl)phenyl]ethyl]pyrrolidine-2-carboxamide

Using General procedure for the acylation of VHL ligands starting from (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide, hydrogen chloride (1:1) (0.42 mmol) and 10-hydroxydecanoic acid as the appropriate acid, 123 mg of the desired product were obtained. ¹H NMR (500 MHz, DMSO-d₆): δ ppm 8.98 (s, 1H), 8.38 (d, 1H), 7.79 (d, 1H), 7.43 (d, 2H), 7.37 (d, 2H), 5.10 (d, 1H), 4.91 (qn, 1H), 4.51 (d, 1H), 4.41 (t, 1H), 4.33 (t, 1H), 4.27 (m, 1H), 3.61/3.58 (dd+dd, 2H), 3.36 (q, 2H), 2.45 (s, 3H), 2.24/2.10 (m+m, 2H), 2.00/1.78 (m+m, 2H), 1.49/1.45 (m+m, 2H), 1.39 (qn, 2H), 1.37 (d, 3H), 1.29-1.19 (m, 10H), 0.93 (s, 9H); ¹³C NMR (125 MHz, DMSO-d₆) δ ppm 172.5, 171.1, 170.0, 152.0, 148.2, 145.1, 131.6, 130.2, 129.3, 126.9, 69.3, 61.2, 59.0, 56.8, 56.7, 48.2, 38.2, 35.7, 35.4, 33.0, 26.9, 25.9, 22.9, 16.5; HRMS-ESI (m/z): [M+H]⁺ calcd for C₃₃H₅₁N₄O₅S: 615.3575. found: 615.3575.

### Step B:[10-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-10-oxo-decyl] 4-methylbenzenesulfonate

Using the General procedure for the tosylation of the hydroxyalkyl VHL ligand-derivatives starting from the product of Step A (100 mg), 90 mg of the desired product were obtained. ¹H NMR (500 MHz, DMSO-d₆): δ ppm 8.98 (s, 1H), 8.38 (d, 1H), 7.79 (d, 1H), 7.78 (d, 2H), 7.48 (d, 2H), 7.43 (d, 2H), 7.38 (d, 2H), 5.10 (brs, 1H), 4.91 (qn, 1H), 4.52 (d, 1H), 4.41 (t, 1H), 4.27 (m, 1H), 4.00 (t, 2H), 3.61/3.58 (dd+dd, 2H), 2.45 (s, 3H), 2.42 (s, 3H), 2.24/2.09 (m+m, 2H), 2.00/1.78 (m+m, 2H), 1.53 (qn, 2H), 1.47/1.42 (m+m, 2H), 1.37 (d, 3H), 1.21-1.11 (m, 10H), 0.93 (s, 9H); ¹³C NMR (125 MHz, DMSO-d₆) δ ppm 172.5, 171.1, 170.0, 152.0, 148.3, 145.3, 145.1, 133.0, 131.6, 130.6, 130.1, 129.3, 128.0, 126.9, 71.4, 69.2, 59.0, 56.8, 56.7, 48.2, 38.2, 35.7, 35.3, 28.6, 26.9, 25.9, 22.9, 21.6, 16.5; HRMS-ESI (m/z): [M+H]⁺ calcd for C₄₀H₅₇N₄O₇S₂: 769.3663. found: 769.3668.

### Step C: 6-[3-(1,3-benzothiazol-2-ylamino)-4-methyl-6,7-dihydro-5H-pyrido[2,3-c]pyridazin-8-yl]-3-[1-[[3-[2-[[10-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-10-oxo-decyl]-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (22 mg) and the product of Step B as the appropriate alkylating agent, 15 mg of the desired product were obtained. HRMS-ESI (m/z): [M+H]⁺ calcd for C₇₄H₉₈N₁₃O₇S₂: 1344.7148. found: 1344.7151.

### Example 134: 2-[3-(1,3-benzothiazol-2-ylamino)-4-methyl-6,7-dihydro-5H-pyrido[2,3-c]pyridazin-8-yl]-5-[3-[2-fluoro-4-[3-[4-[10-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-10-oxo-decyl]piperazin-1-yl]prop-1-ynyl]phenoxy]propyl]thiazole-4-carboxylic acid

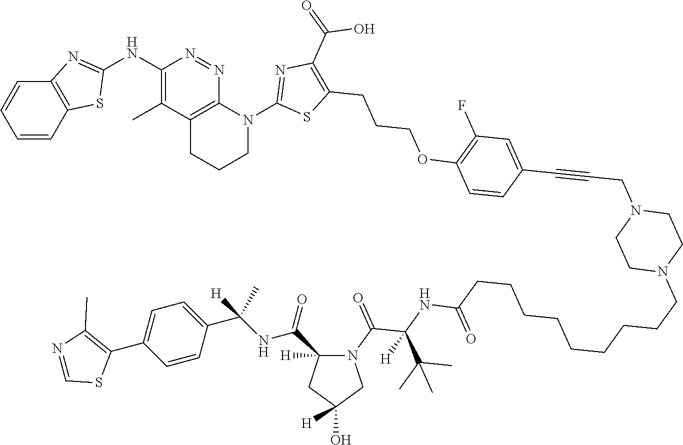 [SMILES: Cc1ncsc1-c1ccc([C@H](C)NC(=O)[C@@H]2C[C@@H](O)CN2C(=O)[C@@H](NC(=O)CCCCCCCCCN2CCN(CC#Cc3ccc(OCCCc4sc(N5CCCc6c5nnc(Nc5nc7ccccc7s5)c6C)nc4C(=O)O)c(F)c3)CC2)C(C)(C)C)cc1]

Using the Degrader Synthesis by Alkylation and Hydrolysis General Procedure starting from the product of Preparation 11 (20 mg) and the product of Step B of Example 133 as the appropriate alkylating agent, 15 mg of the desired product were obtained. HRMS-ESI (m/z): [M+H]⁺ calcd for C₆₈H₈₄FN₁₂O₇S₃: 1295.5726. found: 1295.5720.

### Example 135: 6-[3-(1,3-benzothiazol-2-ylamino)-4-methyl-6,7-dihydro-5H-pyrido[2,3-c]pyridazin-8-yl]-3-[1-[[3-[2-[[11-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-11-oxo-undecyl]-methyl-amino]ethoxy]-5,7-dimethyl-1-adamantyl]methyl]-5-methyl-pyrazol-4-yl]pyridine-2-carboxylic acid

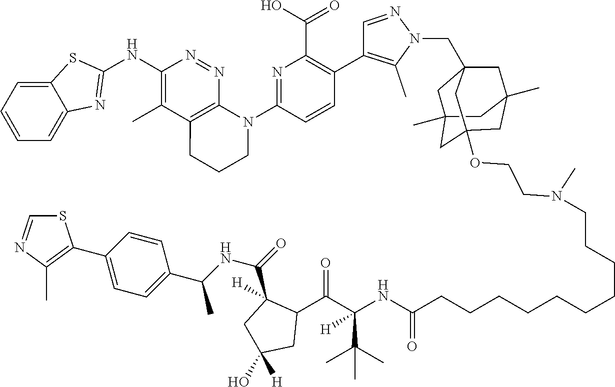 [SMILES: Cc1ncsc1-c1ccc([C@H](C)NC(=O)[C@@H]2C[C@@H](O)CC2C(=O)[C@@H](NC(=O)CCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1]

### Step A: (2S,4R)-4-hydroxy-1-[(2S)-2-(11-hydroxyundecanoylamino)-3,3-dimethyl-butanoyl]-N-[(1S)-1-[4-(4-methylthiazol-5-yl)phenyl]ethyl]pyrrolidine-2-carboxamide

Using General procedure for the acylation of VHL ligands starting from (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide, hydrogen chloride (1:1) (0.42 mmol) and 11-hydroxyundecanoic acid as the appropriate acid, 190 mg of the desired product were obtained. ¹H NMR (500 MHz, DMSO-d₆): δ ppm 8.98 (s, 1H), 8.38 (d, 1H), 7.79 (d, 1H), 7.43 (m, 2H), 7.38 (m, 2H), 5.10 (d, 1H), 4.91 (m, 1H), 4.51 (d, 1H), 4.41 (t, 1H), 4.32 (t, 1H), 4.27 (m, 1H), 3.66-3.54 (m, 2H), 3.36 (m, 2H), 2.45 (s, 3H), 2.24/2.09 (m+m, 2H), 2.00/1.78 (m+m, 2H), 1.60-1.13 (m, 16H), 1.37 (d, 3H), 0.93 (s, 9H); ¹³C NMR (125 MHz, DMSO-d₆) δ ppm 152.0, 129.3, 126.8, 69.2, 61.2, 59.0, 56.8, 53.7, 48.2, 38.2, 35.3, 26.9, 22.9, 16.5; HRMS-ESI (m/z): [M+H]⁺ calcd for C₃₄H₅₃N₄O₅S: 629.3731. found: 629.3735.

### Step B: [11-[[(1S)-1-[(2S,4R)-4-hydroxy-2-[[(1S)-1-[4-(4-methylthiazol-5-ylphenyl]ethyl]carbamoyl]pyrrolidine-1-carbonyl]-2,2-dimethyl-propyl]amino]-11-oxo-undecyl] 4-methyl benzenesulfonate

Using the General procedure for the tosylation of the hydroxyalkyl VHL ligand-derivatives starting from the product of Step A (100 mg), 95 mg of the desired product were obtained. ¹H NMR (500 MHz, DMSO-d₆): δ ppm 8.98 (s, 1H), 8.38 (d, 1H), 7.79 (d, 1H), 7.78 (d, 2H), 7.48 (d, 2H), 7.43 (d, 2H), 7.38 (d, 2H), 5.1 (brs, 1H), 4.92 (qn, 1H), 4.52 (d, 1H), 4.41 (t, 1H), 4.27 (brm, 1H), 4.00 (t, 2H), 3.61/3.58 (dd+dd, 2H), 2.45 (s, 3H), 2.42 (s, 3H), 2.24/2.09 (m+m, 2H), 2.00/1.79 (m+m, 2H), 1.53 (qn, 2H), 1.49/1.43 (m+m, 2H), 1.37 (d, 3H), 1.30-1.10 (m, 12H), 0.93 (s, 9H); ¹³C NMR (125 MHz, DMSO-d₆) δ ppm 152.0, 130.6, 129.3, 128.0, 126.8, 71.4, 69.2, 59.0, 56.8, 56.7, 48.2, 38.2, 35.3, 28.6, 26.9, 25.9, 22.9, 21.6, 16.5; HRMS-ESI (m/z): [M+H]⁺ calcd for C₄₁H₅₉N₄O₇S₂: 783.3820. found: 783.3823.

### Step C: 6-[3-(1,3-benzothiazol-2-ylamino)-4-methyl-6,7-dihydro-5H-pyrido[2,3-c]pyridazin-8-yl]-3-[1-[[3-[2-[[11-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-11-oxo-undecyl]-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (30 mg) and the product of Step B as the appropriate alkylating agent, 16 mg of the desired product were obtained. HRMS-ESI (m/z): [M+H]⁺ calcd for C₇₅H₁₀₀N₁₃O₇S₂: 1358.7305. found: 1358.7306.

### Example 136: 6-[3-(1,3-benzothiazol-2-ylamino)-4-methyl-6,7-dihydro-5H-pyrido[2,3-c]pyridazin-8-yl]-3-[1-[[3-[2-[[13-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-13-oxo-tridecyl]-methyl-amino]ethoxy]-5,7-dimethyl-1-adamantyl]methyl]-5-methyl-pyrazol-4-yl]pyridine-2-carboxylic acid

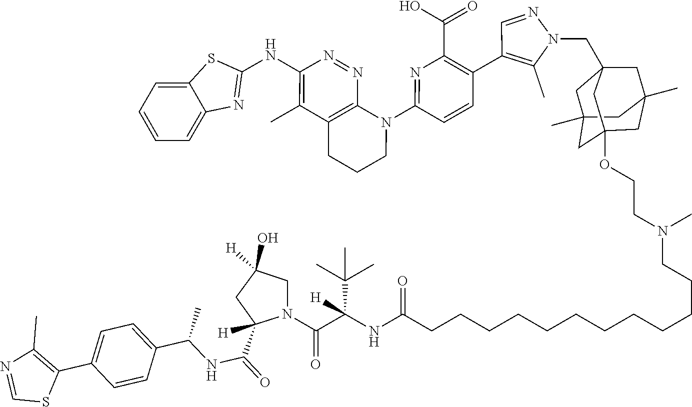 [SMILES: Cc1ncsc1-c1ccc([C@H](C)NC(=O)[C@@H]2C[C@@H](O)CN2C(=O)[C@@H](NC(=O)CCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1]

### Step A: (2S,4R)-4-hydroxy-1-[(2S)-2-(13-hydroxytridecanoylamino)-3,3-dimethyl-butanoyl]-N-[(1S)-1-[4-(4-methylthiazol-5-yl)phenyl]ethyl]pyrrolidine-2-carboxamide

Using General procedure for the acylation of VHL ligands starting from (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide, hydrogen chloride (1:1) (0.42 mmol) and 13-hydroxytridecanoic acid as the appropriate acid, 177 mg of the desired product were obtained. ¹H NMR (500 MHz, DMSO-d₆): δ ppm 8.98 (s, 1H), 8.38 (d, 1H), 7.79 (d, 1H), 7.43 (d, 2H), 7.38 (d, 2H), 5.10 (d, 1H), 4.91 (qn, 1H), 4.51 (d, 1H), 4.41 (t, 1H), 4.32 (t, 1H), 4.27 (m, 1H), 3.61/3.58 (dd+dd, 2H), 3.36 (q, 2H), 2.45 (s, 3H), 2.24/2.09 (m+m, 2H), 2.00/1.78 (m+m, 2H), 1.49/1.45 (m+m, 2H), 1.39 (qn, 2H), 1.37 (d, 3H), 1.28-1.19 (m, 16H), 0.93 (s, 9H); ¹³C NMR (125 MHz, DMSO-d₆) δ ppm 152.0, 129.3, 126.8, 69.2, 61.2, 59.0, 56.8, 56.7, 48.2, 38.2, 36.9, 35.3, 33.0, 25.9, 22.9, 16.5; HRMS-ESI (m/z): [M+H]⁺ calcd for C₃₆H₅₇N₄O₅S: 657.4044. found: 657.4044.

### Step B: [13-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-13-oxo-tridecyl] 4-methylbenzenesulfonate

Using the General procedure for the tosylation of the hydroxyalkyl VHL ligand-derivatives starting from the product of Step A (100 mg), 88 mg of the desired product were obtained. ¹H NMR (500 MHz, DMSO-d₆): δ ppm 8.98 (s, 1H), 8.38 (d, 1H), 7.79 (d, 1H), 7.78 (d, 2H), 7.48 (d, 2H), 7.43 (d, 2H), 7.38 (d, 2H), 5.10 (brs, 1H), 4.91 (qn, 1H), 4.51 (d, 1H), 4.41 (t, 1H), 4.27 (brm, 1H), 3.99 (t, 2H), 3.61/3.58 (dd+dd, 2H), 2.45 (s, 3H), 2.42 (s, 3H), 2.25/2.09 (m+m, 2H), 2.00/1.79 (m+m, 2H), 1.53 (qn, 2H), 1.48/1.44 (m+m, 2H), 1.37 (d, 3H), 1.28-1.10 (m, 16H), 0.93 (s, 9H); ¹³C NMR (125 MHz, DMSO-d₆) δ ppm 152.0, 130.6, 129.3, 128.0, 126.8, 71.4, 69.2, 59, 56.8, 56.7, 48.2, 38.2, 35.4, 28.6, 26.9, 25.9, 22.9, 21.5, 16.5; HRMS-ESI (m/z): [M+H]⁺ calcd for C₄₃H₆₃N₄O₇S₂: 811.4133. found: 811.4140.

### Step C: 6-[3-(1,3-benzothiazol-2-ylamino)-4-methyl-6,7-dihydro-5H-pyrido[2,3-c]pyridazin-8-yl]-3-[1-[[3-[2-[[13-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-13-oxo-tridecyl]-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (30 mg) and the product of Step B as the appropriate alkylating agent, 19 mg of the desired product were obtained. HRMS-ESI (m/z): [M+2H]²⁺ calcd for C₇₇H₁₀₅N₁₃O₇S₂: 693.8845. found: 693.8849.

### Example 137: 6-[3-(1,3-benzothiazol-2-ylamino)-4-methyl-6,7-dihydro-5H-pyrido[2,3-c]pyridazin-8-yl]-3-[1-[[3-[2-[6-[2-[[[(2S,4R)-1-[(2S)-2-[(1-fluorocyclopropanecarbonyl) amino]-3,3-dimethyl-butanoyl]-4-hydroxy-pyrrolidine-2-carbonyl]amino]methyl]-5-(4-methylthiazol-5-yl)phenoxy]hexyl-methyl-amino]ethoxy]-5,7-dimethyl-1-adamantyl]methyl]-5-methyl-pyrazol-4-yl]pyridine-2-carboxylic acid

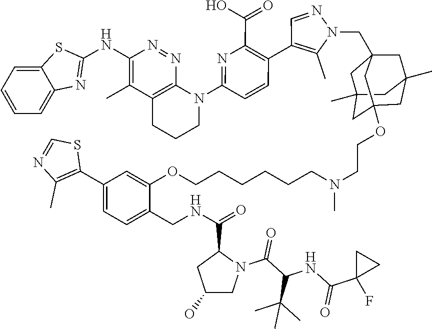

### Step A: (2S,4R)—N-[[2-(6-bromohexoxy)-4-(4-methylthiazol-5-yl)phenyl]methyl]-1-[(2S)-2-[(1-fluorocyclopropanecarbonyl)amino]-3,3-dimethyl-butanoyl]-4-hydroxy-pyrrolidine-2-carboxamide

Using the General procedure for the alkylation of VHL ligand on hydroxy group starting from (2S,4R)-1-[(2S)-2-[(1-fluorocyclopropanecarbonyl)amino]-3,3-dimethyl-butanoyl]-4-hydroxy —N-[[2-hydroxy-4-(4-methylthiazol-5-yl)phenyl]methyl]pyrrolidine-2-carboxamide (0.19 mmol) and 1,6-dibromohexane as the appropriate dibromoalkane, 39 mg of the desired product were obtained. ¹H NMR (500 MHz, DMSO-d₆): δ ppm 8.98 (s, 1H), 8.50 (t, 1H), 7.40 (d, 1H), 7.30 (dd, 1H), 7.00 (d, 1H), 6.95 (dd, 1H), 5.17 (d, 1H), 4.60 (d, 1H), 4.51 (t, 1H), 4.35 (brm, 1H), 4.29/4.20 (dd+dd, 2H), 4.05 (t, 2H), 3.65/3.60 (dd+dd, 2H), 3.54 (t, 2H), 2.45 (s, 3H), 2.08/1.92 (m+m, 2H), 1.83 (qn, 2H), 1.76 (qn, 2H), 1.48 (qn, 2H), 1.47 (qn, 2H), 1.37/1.22 (dd+dd, 4H), 0.96 (s, 9H); ¹³C NMR (125 MHz, DMSO-d₆) δ ppm 151.9, 128.2, 121.2, 112.0, 69.4, 68.0, 59.3, 57.1, 57.0, 38.4, 37.7, 35.5, 32.7, 29.0, 27.8, 26.6, 25.2, 16.5, 13.3; HRMS-ESI (m/z): [M+H]⁺ calcd for C₃₂H₄₅BrFN₄05S: 695.2273. found: 695.2277.

### Step B: 6-[3-(1,3-benzothiazol-2-ylamino)-4-methyl-6,7-dihydro-5H-pyrido[2,3-c]pyridazin-8-yl]-3-[1-[[3-[2-[6-[2-[[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]hexyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (30 mg) and the product of Step A as the appropriate alkylating agent, 13 mg of the desired product were obtained. HRMS-ESI (m/z): [M+H]⁺ calcd for C₇₃H₉₃FN₁₃O₈S₂: 1362.6690. found: 1362.6685.

### Example 138: 6-[3-(1,3-benzothiazol-2-ylamino)-4-methyl-6,7-dihydro-5H-pyrido[2,3-c]pyridazin-8-yl]-3-[1-[[3-[2-[8-[2-[[[(2S,4R)-1-[(2S)-2-[(1-fluorocyclopropanecarbonyl) amino]-3,3-dimethyl-butanoyl]-4-hydroxy-pyrrolidine-2-carbonyl]amino]methyl]-5-(4-methylthiazol-5-yl)phenoxy]octyl-methyl-amino]ethoxy]-5,7-dimethyl-1-adamantyl]methyl]-5-methyl-pyrazol-4-yl]pyridine-2-carboxylic acid

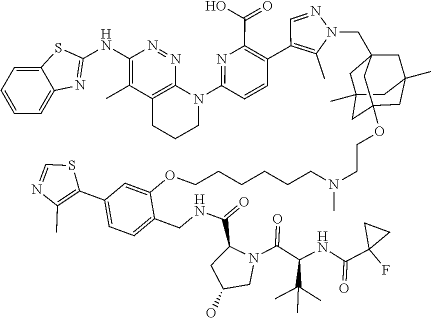

### Step A: (2S,4R)—N-[[2-(8-bromooctoxy)-4-(4-methylthiazol-5-yl)phenyl]methyl]-1-[(2S)-2-[(1-fluorocyclopropanecarbonyl)amino]-3,3-dimethyl-butanoyl]-4-hydroxy-pyrrolidine-2-carboxamide

Using the General procedure for the alkylation of VHL ligand on hydroxy group starting from (2S,4R)-1-[(2S)-2-[(1-fluorocyclopropanecarbonyl)amino]-3,3-dimethyl-butanoyl]-4-hydroxy-N-[[2-hydroxy-4-(4-methylthiazol-5-yl)phenyl]methyl]pyrrolidine-2-carboxamide (0.19 mmol) and 1,8-dibromooctane as the appropriate dibromoalkane, 33 mg of the desired product were obtained. ¹H NMR (500 MHz, DMSO-d₆): δ ppm 8.98 (s, 1H), 8.50 (t, 1H), 7.40 (d, 1H), 7.29 (dd, 1H), 6.99 (d, 1H), 6.94 (dd, 1H), 5.17 (brs, 1H), 4.59 (d, 1H), 4.51 (t, 1H), 4.35 (brm, 1H), 4.29/4.19 (dd+dd, 2H), 4.04 (t, 2H), 3.65/3.60 (dd+dd, 2H), 3.52 (t, 2H), 2.45 (s, 3H), 2.08/1.92 (m+m, 2H), 1.79 (qn, 2H), 1.75 (qn, 2H), 1.46 (qn, 2H), 1.39 (qn, 2H), 1.38/1.22 (dd+dd, 4H), 1.34 (qn, 2H), 1.33 (qn, 2H), 0.96 (s, 9H); ¹³C NMR (125 MHz, DMSO-d₆) δ ppm 151.9, 128.1, 121.1, 112.1, 69.4, 68.1, 59.3, 57.1, 57.0, 38.4, 37.7, 35.7, 32.7, 29.1, 29.1, 28.5, 28.0, 26.6, 26.0, 16.5, 13.5; HRMS-ESI (m/z): [M+H]⁺ calcd for C₃₄H₄₉BrFN₄05S: 723.2586. found: 723.2585.

### Step B: 6-[3-(1,3-benzothiazol-2-ylamino)-4-methyl-6,7-dihydro-5H-pyrido[2,3-c]pyridazin-8-yl]-3-[1-[[3-[2-[8-[2-[[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]octyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (25 mg) and the product of Step A as the appropriate alkylating agent, 22 mg of the desired product were obtained. HRMS-ESI (m/z): [M+H]⁺ calcd for C₇₅H₉₇FN₁₃O₈S₂: 1390.7003. found: 1390.7004.

### Example 139: 6-[3-(1,3-benzothiazol-2-ylamino)-4-methyl-6,7-dihydro-5H-pyrido[2,3-c]pyridazin-8-yl]-3-[1-[[3-[2-[12-[2-[[[(2S,4R)-1-[(2S)-2-[(1-fluorocyclopropanecarbonyl) amino]-3,3-dimethyl-butanoyl]-4-hydroxy-pyrrolidine-2-carbonyl]amino]methyl]-5-(4-methylthiazol-5-yl)phenoxy]dodecyl-methyl-amino]ethoxy]-5,7-dimethyl-1-adamantyl]methyl]-5-methyl-pyrazol-4-yl]pyridine-2-carboxylic acid

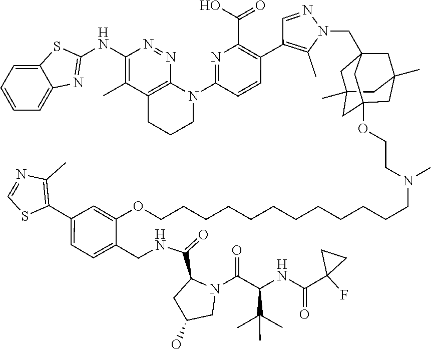

### Step A: (2S,4R)—N-[[2-(12-bromododecoxy)-4-(4-methylthiazol-5-yl)phenyl]methyl]-1-[(2S)-2-[(1-fluorocyclopropanecarbonyl)amino]-3,3-dimethyl-butanoyl]-4-hydroxy-pyrrolidine-2-carboxamide

Using the General procedure for the alkylation of VHL ligand on hydroxy group starting from (2S,4R)-1-[(2S)-2-[(1-fluorocyclopropanecarbonyl)amino]-3,3-dimethyl-butanoyl]-4-hydroxy-N-[[2-hydroxy-4-(4-methylthiazol-5-yl)phenyl]methyl]pyrrolidine-2-carboxamide (0.19 mmol) and 1,12-dibromododecane as the appropriate dibromoalkane, 21 mg of the desired product were obtained. ¹H NMR (500 MHz, DMSO-d₆): δ ppm 8.98 (s, 1H), 8.50 (t, 1H), 7.40 (d, 1H), 7.29 (dd, 1H), 6.99 (d, 1H), 6.94 (dd, 1H), 5.17 (d, 1H), 4.60 (d, 1H), 4.51 (t, 1H), 4.35 (br., 1H), 4.28/4.19 (dd+dd, 2H), 4.04 (t, 2H), 3.65/3.60 (dd+d, 2H), 3.51 (t, 2H), 2.45 (s, 3H), 2.08/1.92 (m+m, 2H), 1.82-1.21 (m, 20H), 1.37/1.22 (m+m, 4H), 0.96 (s, 9H); ¹³C NMR (125 MHz, DMSO-d₆) δ ppm 172.3, 169.4, 168.5, 151.9, 128.1, 121.1, 112.0, 78.6, 69.4, 68.1, 59.3, 57.2, 57.0, 38.4, 37.7, 36.5, 35.7, 26.6, 16.5, 13.3; HRMS-ESI (m/z): [M+H]⁺ calcd for Cs₈H₅₇BrFN₄O₅S: 779.3212. found: 779.3210.

### Step B: 6-[3-(1,3-benzothiazol-2-ylamino)-4-methyl-6,7-dihydro-5H-pyrido[2,3-c]pyridazin-8-yl]-3-[1-[[3-[2-[12-[2-[[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]dodecyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (13 mg) and the product of Step A as the appropriate alkylating agent, 5.5 mg of the desired product were obtained. HRMS-ESI (m/z): [M+H]⁺ calcd for C₇₉H₁₀₅FN₁₃O₈S₂: 1446.7629. found: 1446.7631.

### Example 140: 6-[3-(1,3-benzothiazol-2-ylamino)-4-methyl-6,7-dihydro-5H-pyrido[2,3-c]pyridazin-8-yl]-3-[1-[[3-[2-[12-[2-(2,6-dioxo-3-piperidyl)-1,3-dioxo-isoindolin-5-yl]oxydodecyl-methyl-amino]ethoxy]-5,7-dimethyl-1-adamantyl]methyl]-5-methyl-pyrazol-4-yl]pyridine-2-carboxylic acid

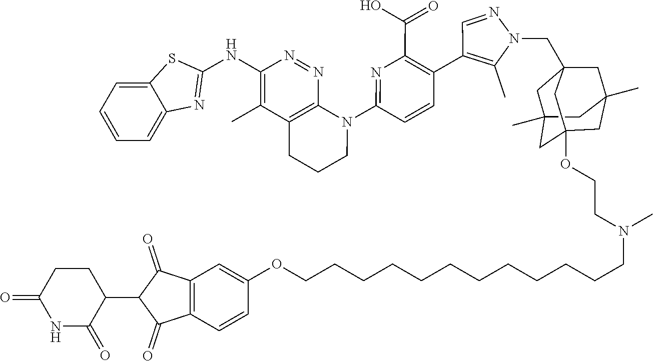

### Step A: 5-(12-bromododecoxy)-2-(2,6-dioxo-3-piperidyl)isoindoline-1,3-dione

Using the General procedure for the alkylation of the 5-hydroxy thalidomide starting from 2-(2,6-dioxo-3-piperidyl)-5-hydroxy-isoindoline-1,3-dione (0.44 mmol) and 1,12-dibromododecane as the appropriate bromoalkane, 96 mg of the desired product were obtained. ¹H NMR (500 MHz, DMSO-d₆): δ ppm 11.12 (s, 1H), 7.82 (d, 1H), 7.42 (d, 1H), 7.34 (dd, 1H), 5.12 (dd, 1H), 4.16 (t, 2H), 3.51 (t, 2H), 2.89/2.59 (td+dd, 2H), 2.53/2.04 (dd+dt, 2H), 1.78 (qn, 2H), 1.74 (qn, 2H), 1.42 (qn, 2H), 1.36 (qn, 2H), 1.34-1.23 (m, 12H); ¹³C NMR (125 MHz, DMSO-d₆) δ ppm 173.3, 170.4, 167.4, 167.3, 164.6, 134.4, 125.8, 123.3, 121.2, 109.3, 69.3, 49.4, 35.7, 32.7, 31.5, 28.8, 28.0, 25.8, 22.5; HRMS-ESI (m/z): [M+H]⁺ calcd for C₂₅H₃₄BrN₂O₅: 521.1646. found: 521.1648.

### Step B: 6-[3-(1,3-benzothiazol-2-ylamino)-4-methyl-6,7-dihydro-5H-pyrido[2,3-c]pyridazin-8-yl]-3-[1-[[3-[2-[12-[2-(2,6-dioxo-3-piperidyl)-1,3-dioxo-isoindolin-5-yl]oxydodecyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (30 mg) and the product of Step A as the appropriate alkylating agent, 27 mg of the desired product were obtained. HRMS-ESI (m/z): [M+H]⁺ calcd for C₆₆H₂N₁₁O₈S: 1188.6063. found: 1188.6061.

### Example 141: 6-[3-(1,3-benzothiazol-2-ylamino)-4-methyl-6,7-dihydro-5H-pyrido[2,3-c]pyridazin-8-yl]-3-[1-[[3-[2-[6-[2-(2,6-dioxo-3-piperidyl)-1,3-dioxo-isoindolin-4-yl]oxyhexyl-methyl-amino]ethoxy]-5,7-dimethyl-1-adamantyl]methyl]-5-methyl-pyrazol-4-yl]pyridine-2-carboxylic acid

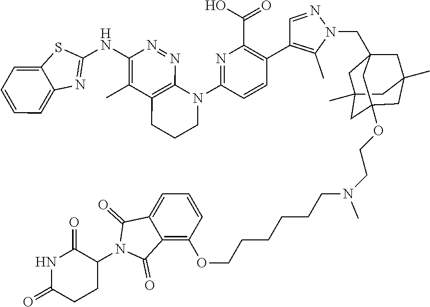

### Step A: 4-(6-bromohexoxy)-2-(2,6-dioxo-3-piperidyl)isoindoline-1,3-dione

Using the General procedure for the alkylation of the 5-hydroxy thalidomide starting from 2-(2,6-dioxo-3-piperidyl)-4-hydroxy-isoindoline-1,3-dione (0.44 mmol) and 1,6-dibromohexane as the appropriate bromoalkane, 52 mg of the desired product were obtained. ¹H NMR (500 MHz, DMSO-d₆): δ ppm 11.11 (s, 1H), 7.81 (dd, 1H), 7.52 (d, 1H), 7.44 (d, 1H), 5.08 (dd, 1H), 4.20 (t, 2H), 3.54 (t, 2H), 2.88/2.59 (ddd+dm, 2H), 2.52/2.02 (m+m, 2H), 1.88-1.40 (m, 8H); ¹³C NMR (125 MHz, DMSO-d₆) δ ppm 173.3/170.4, 167.3/165.8, 156.4, 137.5, 120.3, 115.6, 69.1, 49.2, 35.6, 31.4, 22.5; HRMS-ESI (m/z): [M+H]⁺ calcd for C₁₉H₂₂BrN₂O₅: 437.0707. found: 437.0709.

### Step B: 6-[3-(1,3-benzothiazol-2-ylamino)-4-methyl-6,7-dihydro-5H-pyrido[2,3-c]pyridazin-8-yl]-3-[1-[[3-[2-[6-[2-(2,6-dioxo-3-piperidyl)-1,3-dioxo-isoindolin-4-yl]oxyhexyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (35 mg) and the product of Step A as the appropriate alkylating agent, 25 mg of the desired product were obtained. HRMS-ESI (m/z): [M+H]⁺ calcd for C₆₆H₂N₁₁O₈S: 1104.5124. found: 1104.5113.

### Example 142: 6-[3-(1,3-benzothiazol-2-ylamino)-4-methyl-6,7-dihydro-5H-pyrido[2,3-c]pyridazin-8-yl]-3-[1-[[3-[2-[14-[2-(2,6-dioxo-3-piperidyl)-1,3-dioxo-isoindolin-5-yl]oxytetradecyl-methyl-amino]ethoxy]-5,7-dimethyl-1-adamantyl]methyl]-5-methyl-pyrazol-4-yl]pyridine-2-carboxylic acid

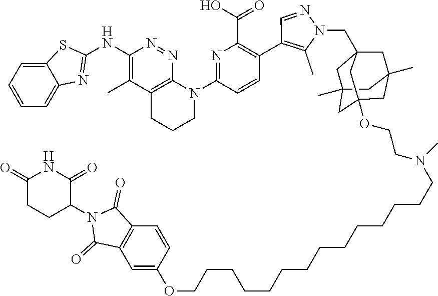

### Step A: 5-(14-bromotetradecoxy)-2-(2,6-dioxo-3-piperidyl)isoindoline-1,3-dione

Using the General procedure for the alkylation of the 5-hydroxy thalidomide starting from 2-(2,6-dioxo-3-piperidyl)-5-hydroxy-isoindoline-1,3-dione (0.44 mmol) and 1,14-dibromotetradecane as the appropriate bromoalkane, 70 mg of the desired product were obtained. ¹H NMR (500 MHz, DMSO-d₆): δ ppm 11.12 (s, 1H), 7.83 (d, 1H), 7.42 (d, 1H), 7.34 (dd, 1H), 5.12 (dd, 1H), 4.16 (t, 2H), 3.51 (t, 2H), 2.89/2.59 (m+m, 2H), 2.53/2.04 (m+m, 2H), 1.83-1.18 (m, 24H); ¹³C NMR (125 MHz, DMSO-d₆) δ ppm 173.3/170.4, 167.4/167.3, 164.6, 125.8, 121.2, 109.3, 69.3, 49.4, 35.7, 31.4, 22.5; HRMS-ESI (m/z): [M+H]⁺ calcd for C₂₇H₈₈BrN₂O₅: 549.1959. found: 549.1962.

### Step B: 6-[3-(1,3-benzothiazol-2-ylamino)-4-methyl-6,7-dihydro-5H-pyrido[2,3-c]pyridazin-8-yl]-3-[1-[[3-[2-[14-[2-(2,6-dioxo-3-piperidyl)-1,3-dioxo-isoindolin-5-yl]oxytetradecyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (30 mg) and the product of Step A as the appropriate alkylating agent, 13 mg of the desired product were obtained. HRMS-ESI (m/z): [M+H]⁺ calcd for C₆₈H₈₆N₁₁O₈S: 1216.6376. found: 1216.6377.

### Example 143: 6-[3-(1,3-benzothiazol-2-ylamino)-4-methyl-6,7-dihydro-5H-pyrido[2,3-c]pyridazin-8-yl]-3-[1-[[3-[2-[8-[2-(2,6-dioxo-3-piperidyl)-1,3-dioxo-isoindolin-4-yl]oxyoctyl-methyl-amino]ethoxy]-5,7-dimethyl-1-adamantyl]methyl]-5-methyl-pyrazol-4-yl]pyridine-2-carboxylic acid

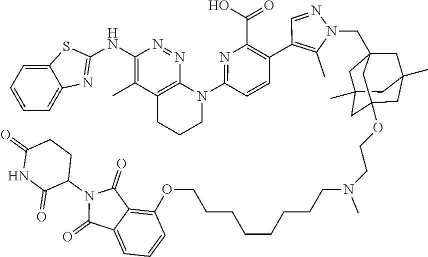

### Step A: 4-(8-bromooctoxy)-2-(2,6-dioxo-3-piperidyl)isoindoline-1,3-dione

Using the General procedure for the alkylation of the 5-hydroxy thalidomide starting from 2-(2,6-dioxo-3-piperidyl)-4-hydroxy-isoindoline-1,3-dione (0.44 mmol) and 1,8-dibromooctane as the appropriate bromoalkane, 140 mg of the desired product were obtained. ¹H NMR (500 MHz, DMSO-d₆): δ ppm 11.11 (s, 1H), 7.80 (dd, 1H), 7.51 (d, 1H), 7.44 (d, 1H), 5.08 (dd, 1H), 4.20 (t, 2H), 3.52 (t, 2H), 2.88/2.58 (ddd+m, 2H), 2.51/2.02 (m+m, 2H), 1.84-1.24 (m, 12H); ¹³C NMR (125 MHz, DMSO-d₆) δ ppm 173.3/170.4, 167.3/165.8, 156.2, 137.5, 120.2, 115.6, 69.2, 49.2, 35.7, 31.4, 22.5; HRMS-ESI (m/z): [M+H]⁺ calcd for C₂₁H₂₆BrN₂O₅: 465.1020. found: 465.1014.

### Step B: 6-[3-(1,3-benzothiazol-2-ylamino)-4-methyl-6,7-dihydro-5H-pyrido[2,3-c]pyridazin-8-yl]-3-[1-[[3-[2-[8-[2-(2,6-dioxo-3-piperidyl)-1,3-dioxo-isoindolin-4-yl]oxyoctyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (30 mg) and the product of Step A as the appropriate alkylating agent, 22 mg of the desired product were obtained. HRMS-ESI (m/z): [M+H]⁺ calcd for C₆₂H₇₄N₁₁O₈S: 1132.5442. found: 1132.5442.

### Example 144: 6-[3-(1,3-benzothiazol-2-ylamino)-4-methyl-6,7-dihydro-5H-pyrido[2,3-c]pyridazin-8-yl]-3-[1-[[3-[2-[10-[2-(2,6-dioxo-3-piperidyl)-1,3-dioxo-isoindolin-4-yl]oxydecyl-methyl-amino]ethoxy]-5,7-dimethyl-1-adamantyl]methyl]-5-methyl-pyrazol-4-yl]pyridine-2-carboxylic acid

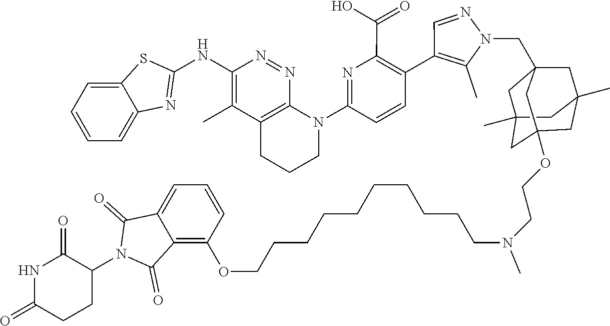

### Step A: 4-(10-bromodecoxy)-2-(2,6-dioxo-3-piperidyl)isoindoline-1,3-dione

Using the General procedure for the alkylation of the 5-hydroxy thalidomide starting from 2-(2,6-dioxo-3-piperidyl)-4-hydroxy-isoindoline-1,3-dione (0.44 mmol) and 1,10-dibromodecane as the appropriate bromoalkane, 79 mg of the desired product were obtained. ¹H NMR (500 MHz, DMSO-d₆): δ ppm 11.11 (s, 1H), 7.80 (dd, 1H), 7.51 (d, 1H), 7.44 (d, 1H), 5.08 (dd, 1H), 4.20 (t, 2H), 3.51 (t, 2H), 2.88/2.59 (m+m, 2H), 2.51/2.02 (m+m, 2H), 1.78 (m, 2H), 1.75 (m, 2H), 1.45 (m, 2H), 1.41-1.21 (m, 10H); ¹³C NMR (125 MHz, DMSO-d₆) δ ppm 137.5, 120.2, 115.6, 69.2, 49.2, 35.7, 32.7, 31.4, 28.9, 24.7, 22.5; HRMS-ESI (m/z): [M+H]⁺ calcd for C₂₃H₃₀BrN₂O₅: 493.1333. found: 493.1332.

### Step B: 6-[3-(1,3-benzothiazol-2-ylamino)-4-methyl-6,7-dihydro-5H-pyrido[2,3-c]pyridazin-8-yl]-3-[1-[[3-[2-[10-[2-(2,6-dioxo-3-piperidyl)-1,3-dioxo-isoindolin-4-yl]oxydecyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (30 mg) and the product of Step A as the appropriate alkylating agent, 16 mg of the desired product were obtained. HRMS-ESI (m/z): [M+H]⁺ calcd for C₆₄H₇₈N₁₁O₈S: 1160.5750. found: 1160.5760.

### Example 145: 6-[3-(1,3-benzothiazol-2-ylamino)-4-methyl-6,7-dihydro-5H-pyrido[2,3-c]pyridazin-8-yl]-3-[1-[[3-[2-[[17-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-17-oxo-heptadecyl]-methyl-amino]ethoxy]-5,7-dimethyl-1-adamantyl]methyl]-5-methyl-pyrazol-4-yl]pyridine-2-carboxylic acid

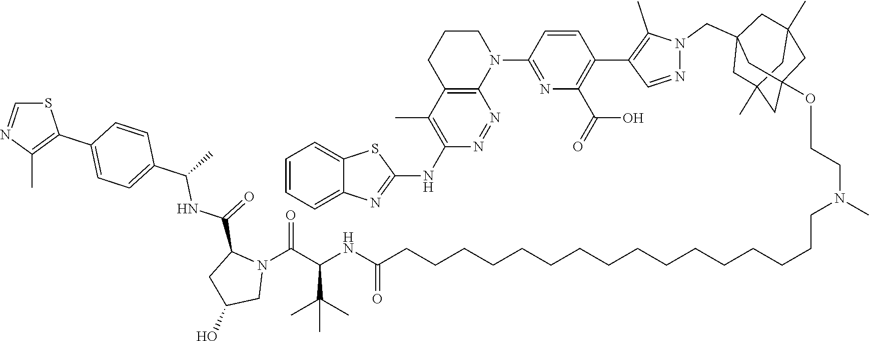 [SMILES: Cc1ncsc1-c1ccc([C@H](C)NC(=O)[C@@H]2C[C@@H](O)CN2C(=O)[C@@H](NC(=O)CCCCCCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1]

### Step A: (2S,4R)-1-[(2S)-2-(17-bromoheptadecanoylamino)-3,3-dimethyl-butanoyl]-4-hydroxy-N-[(1S)-1-[4-(4-methylthiazol-5-yl)phenyl]ethyl]pyrrolidine-2-carboxamide

Using the General procedure for the acylation of VHL ligands starting from (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.42 mmmol) and 17-bromoheptadecanoic acid as the appropriate acid, 255 mg of the desired product were obtained. ¹H NMR (500 MHz, DMSO-d₆): δ ppm 9.98 (s, 1H), 8.38 (d, 1H), 7.79 (d, 1H), 7.43 (d, 2H), 7.38 (d, 2H), 5.10 (brs, 1H), 4.91 (qn, 1H), 4.51 (d, 1H), 4.41 (t, 1H), 4.27 (brm, 1H), 3.61/3.58 (dd+dd, 2H), 3.51 (t, 2H), 2.45 (s, 3H), 2.24/2.069 (m+m, 2H), 2.01/1.79 (m+m, 2H), 1.78 (qn, 2H), 1.50/1.45 (m+m, 2H), 1.39-1.19 (m, 24H), 1.37 (d, 3H), 0.93 (s, 9H); ¹³C NMR (125 MHz, DMSO-d₆) δ ppm 152.0, 129.3, 126.9, 69.2, 59.0, 56.8, 56.7, 48.1, 38.2, 35.7, 35.3, 32.7, 26.9, 25.9, 22.9, 16.5; HRMS-ESI (m/z): [M+H]⁺ calcd for C₄₀H₆₄BrN₄04S: 775.3826. found: 775.3827.

### Step B: 6-[3-(1,3-benzothiazol-2-ylamino)-4-methyl-6,7-dihydro-5H-pyrido[2,3-c]pyridazin-8-yl]-3-[1-[[3-[2-[[17-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-17-oxo-heptadecyl]-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (30 mg) and the product of Step A as the appropriate alkylating agent, 17 mg of the desired product were obtained. HRMS-ESI (m/z): [M+H]⁺ calcd for C₈₁H₁₁₂N₁₃O₇S₂: 1442.8244. found: 1442.8251.

### Example 146: 6-[3-(1,3-benzothiazol-2-ylamino)-4-methyl-6,7-dihydro-5H-pyrido[2,3-c]pyridazin-8-yl]-3-[1-[[3-[2-[14-[2-[[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]tetradecyl-methyl-amino]ethoxy]-5,7-dimethyl-1-adamantyl]methyl]-5-methyl-pyrazol-4-yl]pyridine-2-carboxylic acid

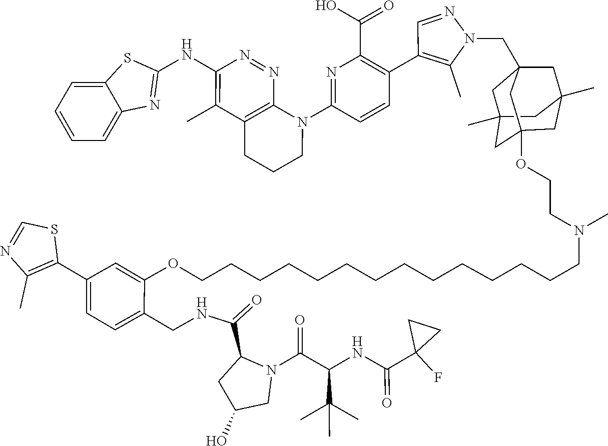

### Step A: (2S,4R)—N-[[2-(14-bromotetradecoxy)-4-(4-methylthiazol-5-yl)phenyl]methyl]-1-[(2S)-2-[(1-fluorocyclopropanecarbonyl)amino]-3,3-dimethyl-butanoyl]-4-hydroxy-pyrrolidine-2-carboxamide

Using the General procedure for the alkylation of VHL ligand on hydroxy group starting from (2S,4R)-1-[(2S)-2-[(1-fluorocyclopropanecarbonyl)amino]-3,3-dimethyl-butanoyl]-4-hydroxy-N-[[2-hydroxy-4-(4-methylthiazol-5-yl)phenyl]methyl]pyrrolidine-2-carboxamide (0.19 mmol) and 1,14-dibromotetradecane as the appropriate dibromoalkane, 33 mg of the desired product were obtained. ¹H NMR (500 MHz, DMSO-d₆): δ ppm 8.98 (s, 1H), 8.50 (t, 1H), 7.40 (d, 1H), 7.29 (dd, 1H), 6.99 (d, 1H), 6.94 (dd, 1H), 5.18 (br., 1H), 4.59 (d, 1H), 4.51 (t, 1H), 4.35 (br., 1H), 4.28/4.19 (dd+dd, 2H), 4.04 (t, 2H), 3.65/3.60 (dd+d, 2H), 3.51 (t, 2H), 2.45 (s, 3H), 2.08/1.92 (m+m, 2H), 1.82-1.20 (m, 24H), 1.37/1.22 (m+m, 4H), 0.96 (s, 9H); ¹³C NMR (125 MHz, DMSO-d₆) δ ppm 172.3, 169.4, 168.5, 151.9, 128.1, 121.1, 112.0, 78.6, 69.4, 68.1, 59.3, 57.2, 57.0, 38.4, 37.7, 36.5, 35.7, 26.6, 16.5, 13.3; HRMS-ESI (m/z): [M+H]⁺ calcd for C₄₀H₆₁BrFN₄O₅S: 807.3524. found: 807.3523.

### Step B: 6-[3-(1,3-benzothiazol-2-ylamino)-4-methyl-6,7-dihydro-5H-pyrido[2,3-c]pyridazin-8-yl]-3-[1-[[3-[2-[14-[2-[[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]tetradecyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (13 mg) and the product of Step A as the appropriate alkylating agent, 11 mg of the desired product were obtained. HRMS-ESI (m/z): [M+H]⁺ calcd for C₈₁H₁₀₉FN₁₃O₈S₂: 1474.7942. found: 1474.7942.

### Example 147: 6-[3-(1,3-benzothiazol-2-ylamino)-4-methyl-6,7-dihydro-5H-pyrido[2,3-c]pyridazin-8-yl]-3-[1-[[3-[2-[16-[2-(2,6-dioxo-3-piperidyl)-1,3-dioxo-isoindolin-5-yl]oxyhexadecyl-methyl-amino]ethoxy]-5,7-dimethyl-1-adamantyl]methyl]-5-methyl-pyrazol-4-yl]pyridine-2-carboxylic acid

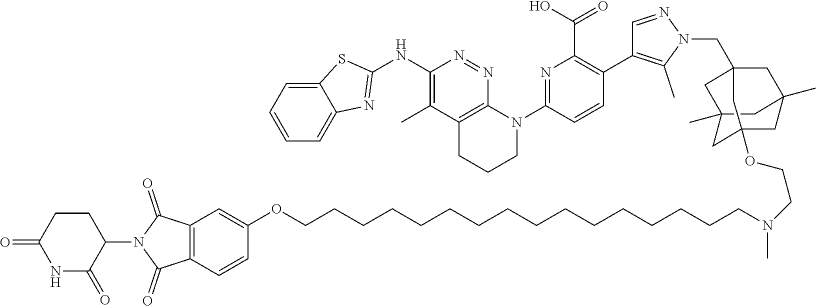 [SMILES: Cc1c(Nc2nc3ccccc3s2)nnc2c1CCCN2c1ccc(-c2cnn(CC34CC5(C)CC(C)(C3)CC(OCCN(C)CCCCCCCCCCCCCCCCOc3ccc6c(c3)C(=O)N(C3CCC(=O)NC3=O)C6=O)(C5)C4)c2C)c(C(=O)O)n1]

### Step A: 5-(16-bromohexadecoxy)-2-(2,6-dioxo-3-piperidyl)isoindoline-1,3-dione

Using the General procedure for the alkylation of the 5-hydroxy thalidomide starting from 2-(2,6-dioxo-3-piperidyl)-5-hydroxy-isoindoline-1,3-dione (0.55 mmol) and 1,16-dibromohexadecane as the appropriate bromoalkane, 45 mg of the desired product were obtained. ¹H NMR (500 MHz, DMSO-d₆): δ ppm 11.12 (s, 1H), 7.83 (d, 1H), 7.42 (d, 1H), 7.35 (dd, 1H), 5.12 (dd, 1H), 4.17 (t, 2H), 3.52 (t, 2H), 2.90/2.60 (td+dd, 2H), 2.54/2.05 (dd+dq, 2H), 1.78 (qn, 2H), 1.75 (qn, 2H), 1.42 (qn, 2H), 1.36-1.21 (m, 20H), 1.35 (qn, 2H); ¹³C NMR (125 MHz, DMSO-d₆) δ ppm 173.3, 170.4, 167.4, 167.3, 164.6, 134.5, 125.8, 123.3, 121.2, 109.3, 69.3, 49.4, 35.8, 32.7, 31.5, 28.8, 28.0, 25.8, 22.6; HRMS-ESI (m/z): [M+H]⁺ calcd for C₂₉H₄₂BrN₂O₅: 577.2272. found: 577.2275.

### Step B: 6-[3-(1,3-benzothiazol-2-ylamino)-4-methyl-6,7-dihydro-5H-pyrido[2,3-c]pyridazin-8-yl]-3-[1-[[3-[2-[16-[2-(2,6-dioxo-3-piperidyl)-1,3-dioxo-isoindolin-5-yl]oxyhexadecyl-methyl-amino]ethoxy]-5,7-dimethyl-1-adamantyl]methyl]-5-methyl-pyrazol-4-yl]pyridine-2-carboxylic acid

Using the Degrader Synthesis by Alkylation and Hydrolysis General Procedure starting from the product of Preparation 4 (45 mg) and the product of Step A as the appropriate alkylating agent, 36 mg of the desired product were obtained. HRMS-ESI (m/z): [M+H]⁺ calcd for C₇₀H₉₀N₁₁O₈S: 1244.6689. found: 1244.6697.

### Example 148: 6-[3-(1,3-benzothiazol-2-ylamino)-4-methyl-6,7-dihydro-5H-pyrido[2,3-c]pyridazin-8-yl]-3-[1-[[3-[2-[[14-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-14-oxo-tetradecyl]-methyl-amino]ethoxy]-5,7-dimethyl-1-adamantyl]methyl]-5-methyl-pyrazol-4-yl]-N,N-dimethyl-pyridine-2-carboxamide

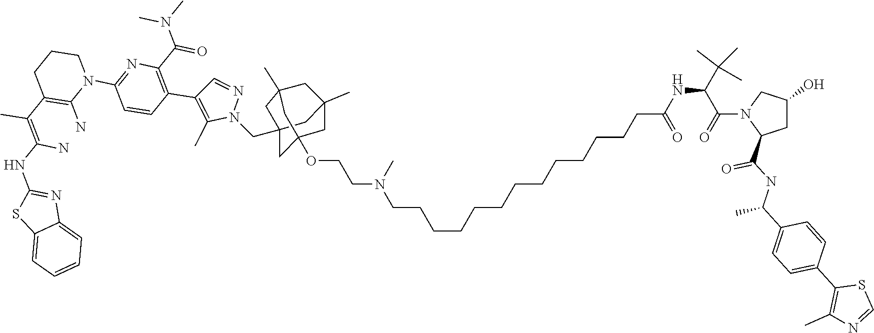 [SMILES: C/C(C1=C(N)N(c2ccc(-c3cnn(CC45CC6(C)CC(C)(C4)CC(OCCN(C)CCCCCCCCCCCCCC(=O)N[C@H](C(=O)N4C[C@H](O)C[C@H]4C(=O)N[C@@H](C)c4ccc(-c7scnc7C)cc4)C(C)(C)C)(C6)C5)c3C)c(C(=O)N(C)C)n2)CCC1)=C(/N)Nc1nc2ccccc2s1]

To the mixture of 48 mg (0.034 mmol) of the product of Example 4 and 0.024 mL (5 eq) of triethylamine in 2 ml of DMF were added 16 mg (1.2 eq) of HATU. After 20 minutes, 0.034 mL (2 eq) of dimethylamine was also added. Then, the reaction was stirred for 2 h. After pouring the reaction into water, the precipitated solid was filtered out, washed with water, dried, and purified by column chromatography to give the desired compound (82%). HRMS-ESI (m/z): [M+H]⁺ calcd for C₈₀H₁₁₁N₁₄O₆S₂: 1427.8246. found: 1427.8256.

### Example 149: 2-[[6-(1,3-benzothiazol-2-ylamino)-5-methyl-pyridazin-3-yl]-[4-[3-[2-[2-[2-[2-[[2-(2,6-dioxo-3-piperidyl)-1,3-dioxo-isoindolin-4-yl]amino]ethoxy]ethoxy]ethoxy]ethoxy]propanoylamino]butyl]amino]-5-[3-[4-[3-(dimethylamino)prop-1-ynyl]-2-fluoro-phenoxy]propyl]thiazole-4-carboxylic acid

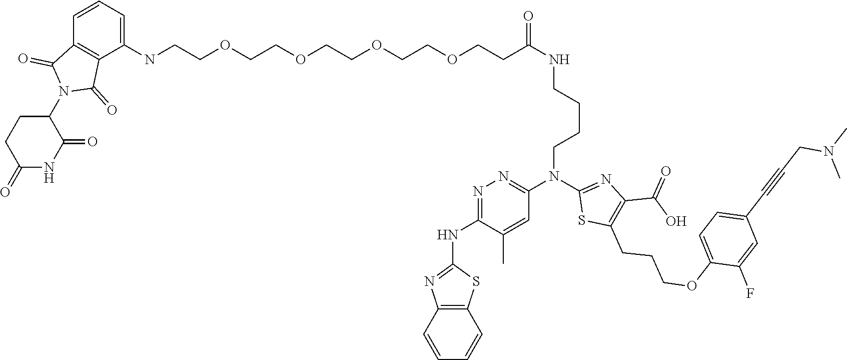 [SMILES: Cc1cc(N(CCCCNC(=O)CCOCCOCCOCCOCCNc2cccc3c2C(=O)N(C2CCC(=O)NC2=O)C3=O)c2nc(C(=O)O)c(CCCOc3ccc(C#CCN(C)C)cc3F)s2)nnc1Nc1nc2ccccc2s1]

### Step A: 3-[2-[2-[2-[2-[[2-(2,6-dioxo-3-piperidyl)-1,3-dioxo-isoindolin-4-yl]amino]ethoxy]ethoxy]ethoxy]ethoxy]propanoic acid

Using General procedure for the nucleophilic substitution of fluoro-thalidomide starting from tert-butyl 3-[2-[2-[2-(2-aminoethoxy)ethoxy]ethoxy]ethoxy]propanoate as the appropriate amine and the TFA deprotection method of the General procedure for the acylation and deprotection of VHL ligands, the desired product was obtained. ¹H NMR (500 MHz, dmso-d6) δ ppm 11.64 (brs, 1H), 11.10 (s, 1H), 7.58 (dd, 1H), 7.14 (d, 1H), 7.04 (d, 1H), 6.61 (brs, 1H), 5.05 (dd, 1H), 3.62 (t, 2H), 3.58 (t, 2H), 3.57-3.46 (m, 12H), 3.47 (t, 2H), 2.88/2.59 (td+dd, 2H), 2.52/2.02 (dd+dt, 2H), 2.43 (t, 2H); ¹³C NMR (500 MHz, dmso-d6) δ ppm 173.3, 173.1, 170.6, 169.4, 167.8, 146.9, 136.7, 132.6, 117.9, 111.2, 109.7, 70.3/70.2/70.1, 69.4, 66.7, 49.0, 42.1, 35.2, 31.4, 22.6; HRMS-ESI (m/z): [M+H]⁺ calcd for C₂₄H₃₂N₃O₁₀: 522.2082. found 522.2087.

### Step B: 2-[[6-(1,3-benzothiazol-2-ylamino)-5-methyl-pyridazin-3-yl]-[4-[3-[2-[2-[2-[2-[[2-(2,6-dioxo-3-piperidyl)-1,3-dioxo-isoindolin-4-yl]amino]ethoxy]ethoxy]ethoxy]ethoxy]propanoylamino]butyl]amino]-5-[3-[4-[3-(dimethylamino)prop-1-ynyl]-2-fluoro-phenoxy]propyl]thiazole-4-carboxylic acid

After stirring the product of Step A (1.5 eq), DIPEA (10 eq), and TSTU (1.7 eq) in DMF (10 mL/mmol) for 20 min, Preparation 18 (1 eq) was added. Then, the reaction mixture was stirred for 24 h. The product was purified by preparative HPLC to give 9.5 mg of the desired product. HRMS-ESI (m/z): [M+H]⁺ calcd for C₅₈H₆₇FN₁₁O₁₂S₂ 1192.4391. found: 1192.4434.

### Example 150: 6-[[6-(1,3-benzothiazol-2-ylamino)-5-methyl-pyridazin-3-yl]-[5-[4-[7-[[2-(2,6-dioxo-3-piperidyl)-1,3-dioxo-isoindolin-4-yl]amino]heptyl]triazol-1-yl]pentyl]amino]-3-[1-[[3-[2-(dimethylamino)ethoxy]-5,7-dimethyl-1-adamantyl]methyl]-5-methyl-pyrazol-4-yl]pyridine-2-carboxylic acid

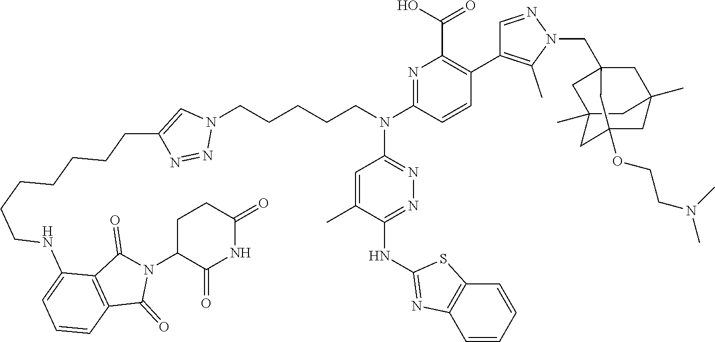 [SMILES: Cc1cc(N(CCCCCn2cc(CCCCCCCNc3cccc4c3C(=O)N(C3CCC(=O)NC3=O)C4=O)nn2)c2ccc(-c3cnn(CC45CC6(C)CC(C)(C4)CC(OCCN(C)C)(C6)C5)c3C)c(C(=O)O)n2)nnc1Nc1nc2ccccc2s1]

### Step A: 2-(2,6-dioxo-3-piperidyl)-4-(non-8-ynylamino)isoindoline-1,3-dione

Using General procedure for the nucleophilic substitution of fluoro-thalidomide starting from non-8-yn-1-amine as the appropriate amine, 93 mg of the desired product were obtained. ¹H NMR (400 MHz, dmso-d6) δ ppm 11.06 (s, 1H), 7.56 (dd, 1H), 7.06 (2d, 2H), 6.52 (t, 1H), 5.04 (dd, 1H), 3.27 (quad, 2H), 2.88 (m, 1H), 2.71 (t, 1H), 2.51 (m, 2H), 2.15 (td, 2H), 2.02 (m, 1H), 1.59 (m, 2H), 1.44 (m, 2H), 1.33 (m, 6H).

### Step B: 6-[[6-(1,3-benzothiazol-2-ylamino)-5-methyl-pyridazin-3-yl]-[5-[4-[7-[[2-(2,6-dioxo-3-piperidyl)-1,3-dioxo-isoindolin-4-yl]amino]heptyl]triazol-1-yl]pentyl]amino]-3-[1-[[3-[2-(dimethylamino)ethoxy]-5,7-dimethyl-1-adamantyl]methyl]-5-methyl-pyrazol-4-yl]pyridine-2-carboxylic acid

Using the procedure described in Example 61, starting from Preparation 16 and the product of Step A, afforded 2.4 mg of the desired product. HRMS-ESI (m/z): [M+2H]²⁺ calcd for C₆₆H₈₃N₁₅O₇S: 614.8155. found: 614.8122.

### Example 151: 6-[[6-(1,3-benzothiazol-2-ylamino)-5-methyl-pyridazin-3-yl]-[5-[4-[[3-[[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-3-oxo-propoxy]methyl]triazol-1-yl]pentyl]amino]-3-[1-[[3-[2-(dimethylamino)ethoxy]-5,7-dimethyl-1-adamantyl]methyl]-5-methyl-pyrazol-4-yl]pyridine-2-carboxylic acid

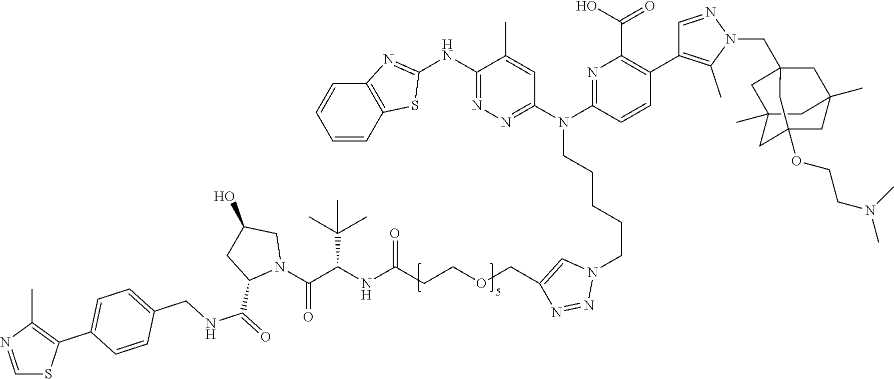

### Step A: (2S,4R)-1-[(2S)-3,3-dimethyl-2-[3-[2-[2-[2-(2-prop-2-ynoxyethoxy)ethoxy]ethoxy] ethoxy]propanoylamino]butanoyl]-4-hydroxy-N-[[4-(4-methylthiazol-5-yl)phenyl]methyl]pyrrolidine-2-carboxamide

Using General procedure for the acylation of VHL ligands starting from (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide and 3-[2-[2-[2-(2-prop-2-ynoxyethoxy)ethoxy]ethoxy]ethoxy]propanoic acid as the appropriate acid, the desired product was obtained.

### Step B: 6-[[6-(1,3-benzothiazol-2-ylamino)-5-methyl-pyridazin-3-yl]-[5-[4-[[3-[[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-3-oxo-propoxy]methyl]triazol-1-yl]pentyl]amino]-3-[1-[[3-[2-(dimethylamino)ethoxy]-5,7-dimethyl-1-adamantyl]methyl]-5-methyl-pyrazol-4-yl]pyridine-2-carboxylic acid

Using the procedure described in Example 61, starting from Preparation 16 and the product of Step A, afforded 4.5 mg of the desired product. HRMS-ESI (m/z): [M+H]⁺ calcd for C₈₀H₁₀₉N₁₆O₁₂S₂: 1549.7852. found: 1549.7824.

### Example 152: 6-[[6-(1,3-benzothiazol-2-ylamino)-5-methyl-pyridazin-3-yl]-[5-[4-[9-[[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-9-oxo-nonyl]triazol-1-yl]pentyl]amino]-3-[1-[[3-[2-(dimethylamino)ethoxy]-5,7-dimethyl-1-adamantyl]methyl]-5-methyl-pyrazol-4-yl]pyridine-2-carboxylic acid

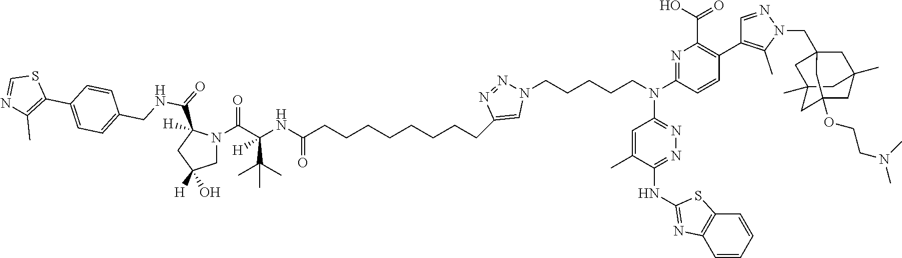 [SMILES: Cc1cc(N(CCCCCn2cc(CCCCCCCCC(=O)N[C@H](C(=O)N3C[C@H](O)C[C@H]3C(=O)NCc3ccc(-c4scnc4C)cc3)C(C)(C)C)nn2)c2ccc(-c3cnn(CC45CC6(C)CC(C)(C4)CC(OCCN(C)C)(C6)C5)c3C)c(C(=O)O)n2)nnc1Nc1nc2ccccc2s1]

### Step A: (2S,4R)-1-[(2S)-3,3-dimethyl-2-(undec-10-ynoylamino)butanoyl]-4-hydroxy-N-[[4-(4-methylthiazol-5-yl)phenyl]methyl]pyrrolidine-2-carboxamide

Using General procedure for the acylation of VHL ligands starting from (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide and undec-10-ynoic acid as the appropriate acid, 17 mg of the desired product were obtained.

### Step B: 6-[[6-(1,3-benzothiazol-2-ylamino)-5-methyl-pyridazin-3-yl]-[5-[4-[9-[[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-9-oxo-nonyl]triazol-1-yl]pentyl]amino]-3-[1-[[3-[2-(dimethylamino)ethoxy]-5,7-dimethyl-1-adamantyl]methyl]-5-methyl-pyrazol-4-yl]pyridine-2-carboxylic acid

Using the procedure described in Example 61, starting from Preparation 16 and the product of Step A, afforded 4.6 mg of the desired product. HRMS-ESI (m/z): [M+H]⁺ calcd for C₇₇H₁₀₃N₁₆O₇S₂: 1427.7637. found: 1427.7638.

### Example 153: 6-[[6-(1,3-benzothiazol-2-ylamino)-5-methyl-pyridazin-3-yl]-[5-[4-[2-[2-[2-[2-[[2-(2,6-dioxo-3-piperidyl)-1,3-dioxo-isoindolin-4-yl]amino]ethoxy]ethoxy]ethoxy]ethoxymethyl]triazol-1-yl]pentyl]amino]-3-[1-[[3-[2-(dimethylamino)ethoxy]-5,7-dimethyl-1-adamantyl]methyl]-5-methyl-pyrazol-4-yl]pyridine-2-carboxylic acid

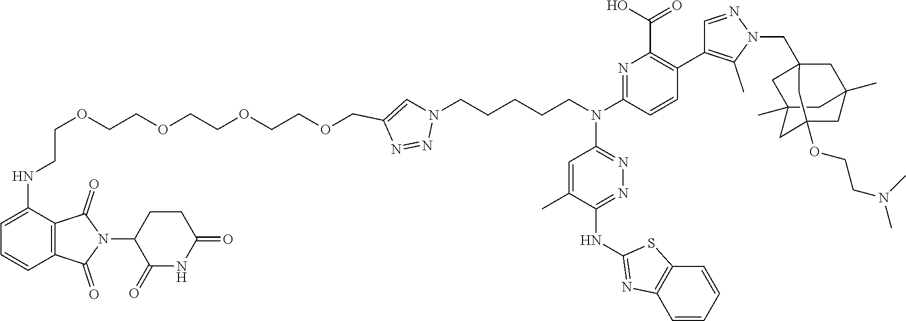 [SMILES: Cc1cc(N(CCCCCn2cc(COCCOCCOCCOCCNc3cccc4c3C(=O)N(C3CCC(=O)NC3=O)C4=O)nn2)c2ccc(-c3cnn(CC45CC6(C)CC(C)(C4)CC(OCCN(C)C)(C6)C5)c3C)c(C(=O)O)n2)nnc1Nc1nc2ccccc2s1]

### Step A: 2-(2,6-dioxo-3-piperidyl)-4-[2-[2-[2-(2-prop-2-ynoxyethoxy)ethoxy]ethoxy]ethylamino]isoindoline-1,3-dione

Using General procedure for the nucleophilic substitution of fluoro-thalidomide starting from 2-[2-[2-(2-prop-2-ynoxyethoxy)ethoxy]ethoxy]ethanamine as the appropriate amine, 104 mg of the desired product were obtained. ¹H NMR (400 MHz, dmso-d6) δ ppm 11.05 (s, 1H), 7.60 (dd, 1H), 7.15 (d, 1H), 7.04 (d, 1H), 6.59 (t, 1H), 5.04 (dd, 1H), 4.12 (d, 2H), 3.53 (m, 16H), 3.40 (t, 1H), 2.88 (m, 1H), 2.58 (m, 2H), 2.02 (m, 1H).

### Step B: 6-[[6-(1,3-benzothiazol-2-ylamino)-5-methyl-pyridazin-3-yl]-[5-[4-[2-[2-[2-[2-[[2-(2,6-dioxo-3-piperidyl)-1,3-dioxo-isoindolin-4-yl]amino]ethoxy]ethoxy]ethoxy]ethoxymethyl] triazol-1-yl]pentyl]amino]-3-[1-[[3-[2-(dimethylamino)ethoxy]-5,7-dimethyl-1-adamantyl]methyl]-5-methyl-pyrazol-4-yl]pyridine-2-carboxylic acid

Using the procedure described in Example 61, starting from Preparation 16 and the product of Step A, afforded 2.9 mg of the desired product. HRMS-ESI (m/z): [M+Na]⁺ calcd for C₆₈H₈₅N₁₅O₁₁SNa: 1342.6165. found: 1342.6078.

### Example 154: 6-[3-(1,3-benzothiazol-2-ylamino)-4-methyl-6,7-dihydro-5H-pyrido[2,3-c]pyridazin-8-yl]-3-[1-[[3-[2-[[14-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-14-oxo-tetradecyl]-methyl-amino]ethoxy]-5,7-dimethyl-1-adamantyl]methyl]-5-methyl-pyrazol-4-yl]pyridine-2-carboxamide

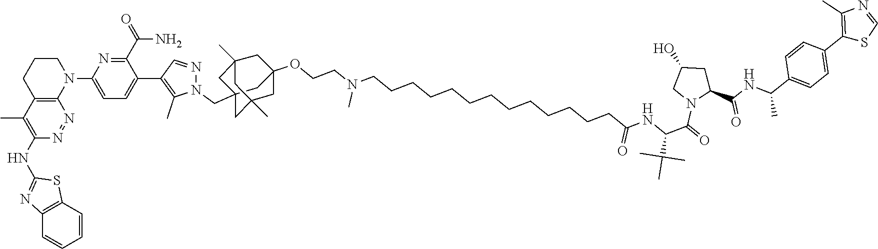 [SMILES: Cc1ncsc1-c1ccc([C@H](C)NC(=O)[C@@H]2C[C@@H](O)CN2C(=O)[C@@H](NC(=O)CCCCCCCCCCCCCN(C)CCOC23CC4(C)CC(C)(CC(Cn5ncc(-c6ccc(N7CCCc8c7nnc(Nc7nc9ccccc9s7)c8C)nc6C(N)=O)c5C)(C4)C2)C3)C(C)(C)C)cc1]

### Step A: 6-[3-(1,3-benzothiazol-2-ylamino)-4-methyl-6,7-dihydro-5H-pyrido[2,3-c]pyridazin-8-yl]-3-[1-[[3-(2-hydroxyethoxy)-5,7-dimethyl-1-adamantyl]methyl]-5-methyl-pyrazol-4-yl]pyridine-2-carboxylic acid

100 mg of Preparation 4, Step F in 1 mL of acetonitrile was treated with 0.45 mmol of KOH. Then, the mixture was stirred at 60° C. for 1 h and the product was purified by preparative reversed phase chromatography to give the desired product (55%). ¹H NMR (500 MHz, dmso-d6) δ ppm 7.89 (d, 1H), 7.81 (brd., 1H), 7.65 (d, 1H), 7.50 (br., 1H), 7.42 (s, 1H), 7.35 (t, 1H), 7.17 (t, 1H), 4.02 (t, 2H), 3.87 (s, 2H), 3.41 (t, 2H), 3.35 (t, 2H), 2.86 (t, 2H), 2.32 (s, 3H), 2.21 (s, 3H), 1.99 (m, 2H), 1.46-0.95 (m, 12H), 0.87 (s, 6H); ¹³C NMR (125 MHz, dmso-d6) δ ppm 168.9, 139.6, 137.8, 126.4, 122.4, 122.1, 118.1, 62.2, 61.5, 59.0, 45.4, 30.2, 24.3, 21.7, 12.6, 11.1; HRMS-ESI (m/z): [M+H]⁺ calcd for C₄₀H₄₇N₈O₄S: 735.3435. found: 735.3438.

### Step B: 6-[3-(1,3-benzothiazol-2-ylamino)-4-methyl-6,7-dihydro-5H-pyrido[2,3-c]pyridazin-8-yl]-3-[1-[[3-(2-hydroxyethoxy)-5,7-dimethyl-1-adamantyl]methyl]-5-methyl-pyrazol-4-yl]pyridine-2-carboxamide

To 200 mg of the product of Step A and 88 mg (1.5 eq) of (Boc)₂O in 3 mL of 1,4-dioxane was added 0.027 mL of pyridine and the mixture was stirred for 10 minutes. After the addition of 32 mg (1.5 eq) of NH₄HCO₃, the mixture was stirred for 5 days and purified by column chromatography using DCM and MeOH as eluents on silica gel to give the desired product (63%). ¹H NMR (500 MHz, dmso-d6) δ ppm 7.90 (d, 1H), 7.82 (brs, 1H), 7.78/7.35 (d+d, 2H), 7.61 (d, 1H), 7.53 (brs, 1H), 7.42 (s, 1H), 7.35 (m, 1H), 7.16 (m, 1H), 4.47 (t, 1H), 4.07 (m, 2H), 3.85 (s, 2H), 3.41 (m, 2H), 3.35 (t, 2H), 2.86 (t, 2H), 2.32 (s, 3H), 2.18 (s, 3H), 1.99 (m, 2H), 1.43-0.97 (m, 12H), 0.87 (s, 6H); ¹³C NMR (125 MHz, dmso-d6) δ ppm 140.3, 138.0, 126.4, 122.4, 122.0, 118.1, 62.1, 61.6, 58.9, 45.4, 30.2, 24.4, 21.7, 12.6, 11.3; HRMS-ESI (m/z): [M+H]⁺ calcd for C₄₀H₄₈N₉O₃S: 734.3595. found: 734.3595.

### Step C: 2-[[3-[[4-[6-[3-(1,3-benzothiazol-2-ylamino)-4-methyl-6,7-dihydro-5H-pyrido[2,3-c]pyridazin-8-yl]-2-carbamoyl-3-pyridyl]-5-methyl-pyrazol-1-yl]methyl]-5,7-dimethyl-1-adamantyl]oxy]ethyl 4-methylbenzenesulfonate

To 120 mg of the product of Step B in 3 mL of DCM were added 0.11 mL (4.8 eq) of triethylamine and 250 mg of p-tolylsulfonyl 4-methylbenzenesulfonate and the mixture was stirred for 4 days and purified by column chromatography using DCM and MeOH as eluents on silica gel to give the desired product (44%). ¹H NMR (500 MHz, dmso-d6) δ ppm 12.20/10.83 (brs/brs, 1H), 7.90 (d, 1H), 7.82 (br, 1H), 7.77 (d, 2H), 7.77/7.34 (s+s, 2H), 7.62 (d, 1H), 7.60 (br, 1H), 7.45 (d, 2H), 7.43 (s, 1H), 7.35 (t, 1H), 7.17 (t, 1H), 4.07 (t, 2H), 4.06 (t, 2H), 3.83 (s, 2H), 3.49 (t, 2H), 2.86 (t, 2H), 2.40 (s, 3H), 2.32 (s, 3H), 2.17 (s, 3H), 1.99 (qn, 2H), 1.29 (s, 2H), 1.15/1.11 (d+d, 4H), 1.12/1.09 (d+d, 4H), 1.01/0.97 (d+d, 2H), 0.83 (s, 6H); ¹³C NMR (125 MHz, dmso-d6) δ ppm 140.2, 137.9, 130.6, 128.2, 126.3, 122.3, 122.1, 118.1, 71.5, 58.9, 58.4, 49.9, 46.6, 45.8, 45.4, 42.9, 30.1, 24.4, 21.8, 21.6, 12.7, 11.3; HRMS-ESI (m/z): [M+H]⁺ calcd for C₄₇H₅₄N₉O₅S₂: 888.3684. found: 888.3685.

### Step D: 6-[3-(1,3-benzothiazol-2-ylamino)-4-methyl-6,7-dihydro-5H-pyrido[2,3-c]pyridazin-8-yl]-3-[1-[[3-[2-[[14-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-14-oxo-tetradecyl]-methyl-amino]ethoxy]-5,7-dimethyl-1-adamantyl]methyl]-5-methyl-pyrazol-4-yl]pyridine-2-carboxamide

The mixture of the product of Step C (58 mg) and Preparation 20 (79 mg, 1.8 eq) in 5 mL of acetonitrile was stirred at 60° C. for 2 days. Purification by preparative reversed phase chromatography afforded the desired product (36%). HRMS-ESI (m/z): [M+2H]²⁺ calcd for C₇H₁₀₈N₁₄O₆S₂: 700.4003. found: 700.4001.

### Example 155: (2S,4R)-1-[(2S)-2-[14-[2-[[3-[[4-[6-[3-(1,3-benzothiazol-2-ylamino)-4-methyl-6,7-dihydro-5H-pyrido[2,3-c]pyridazin-8-yl]-2-(hydroxymethyl)-3-pyridyl]-5-methyl-pyrazol-1-yl]methyl]-5,7-dimethyl-1-adamantyl]oxy]ethyl-methyl-amino]tetradecanoylamino]-3,3-dimethyl-butanoyl]-4-hydroxy-N-[(1S)-1-[4-(4-methylthiazol-5-yl)phenyl]ethyl]pyrrolidine-2-carboxamide

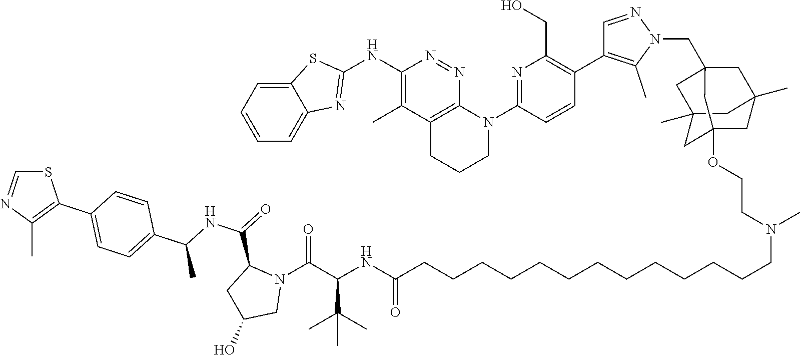 [SMILES: Cc1ncsc1-c1ccc([C@H](C)NC(=O)[C@@H]2C[C@@H](O)CN2C(=O)[C@@H](NC(=O)CCCCCCCCCCCCCN(C)CCOC23CC4(C)CC(C)(CC(Cn5ncc(-c6ccc(N7CCCc8c7nnc(Nc7nc9ccccc9s7)c8C)nc6CO)c5C)(C4)C2)C3)C(C)(C)C)cc1]

### Step A: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3-[2-[[14-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-14-oxo-tetradecyl]-methyl-amino]ethoxy]-5,7-dimethyl-1-adamantyl]methyl]-5-methyl-pyrazol-4-yl]pyridine-2-carboxylate

To the product of Preparation 4 (0.17 mmol) and DIPEA (0.17 mL) in acetonitrile (2 mL) was added the product of Step B of Example 4 (1.5 eq) and the mixture was stirred at 60° C. for 3 days. The product was purified by column chromatography to give the desired product (56%). ¹H NMR (400 MHz, dmso-d6) δ ppm 8.98 (s, 1H), 8.37 (d, 1H), 7.95 (d, 1H), 7.80 (d, 1H), 7.78 (d, 1H), 7.67 (d, 1H), 7.49 (br., 1H), 7.43 (d, 2H), 7.39 (s, 1H), 7.37 (d, 2H), 7.33 (t, 1H), 7.19 (dm, 2H), 7.16 (t, 1H), 6.90 (dm, 2H), 5.10 (s, 2H), 5.10 (brs., 1H), 4.91 (m, 1H), 4.51 (d, 1H), 4.41 (t, 1H), 4.27 (br., 1H), 3.99 (m, 2H), 3.84 (s, 2H), 3.74 (s, 3H), 3.61/3.58 (dd+dd, 2H), 3.39 (t, 2H), 2.84 (t, 2H), 2.45 (s, 3H), 2.36 (t, 2H), 2.32 (s, 3H), 2.25 (t, 2H), 2.23/2.08 (m+m, 2H), 2.12 (s, 3H), 2.11 (s, 3H), 2.00/1.78 (m+m, 2H), 1.98 (m, 2H), 1.41-0.90 (m, 12H), 1.37 (d, 3H), 0.92 (s, 9H), 0.84 (s, 6H); ¹³C NMR (400 MHz, dmso-d6) δ ppm 152.0, 139.9, 137.7, 130.2, 129.3, 126.8, 126.8, 122.4, 122.1, 118.9, 114.3, 69.2, 66.7, 59.1, 59.0, 58.7, 57.9, 57.8, 56.8, 56.7, 55.6, 48.2, 45.4, 43.1, 38.2, 35.3, 30.1, 26.9, 24.3, 22.9, 21.7, 16.5, 12.6, 10.9; HRMS-ESI (m/z): [M+2H]²⁺ calcd for C₈₆H₁₁₅N₁₃O₈S₂: 760.9211. found: 760.9211.

### Step B: (2S,4R)-1-[(2S)-2-[14-[2-[[3-[[4-[6-[3-(1,3-benzothiazol-2-ylamino)-4-methyl-6,7-dihydro-5H-pyrido[2,3-c]pyridazin-8-yl]-2-(hydroxymethyl)-3-pyridyl]-5-methyl-pyrazol-1-yl]methyl]-5,7-dimethyl-1-adamantyl]oxy]ethyl-methyl-amino]tetradecanoylamino]-3,3-dimethyl-butanoyl]-4-hydroxy-N-[(1S)-1-[4-(4-methylthiazol-5-yl)phenyl]ethyl]pyrrolidine-2-carboxamide

To the solution of 125 mg (0.082 mmol) of the product of Step A in 3 ml of THF was added dropwise 0.16 mL (0.16 mmol) of a 1 M solution of LiAlH₄ and the reaction was stirred for 0.5 h. After the reaction was quenched with 6 mL of saturated potassium sodium tartrate (aqueous), the mixture was extracted with EtOAc (2×10 mL). The combined organic layers were dried, concentrated and purified by preparative reversed phase chromatography to give the desired compound (20%). HRMS-ESI (m/z): [M+2H]²⁺ calcd for C₇₈H₁₀₉N₁₃O₆S₂: 693.9027. found: 693.9030.

### Example 156: 6-[3-(1,3-benzothiazol-2-ylamino)-4-methyl-6,7-dihydro-5H-pyrido[2,3-c]pyridazin-8-yl]-3-[1-[[3-[2-[[14-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-14-oxo-tetradecyl]-methyl-amino]ethoxy]-5,7-dimethyl-1-adamantyl]methyl]-5-methyl-pyrazol-4-yl]-N-methyl-pyridine-2-carboxamide

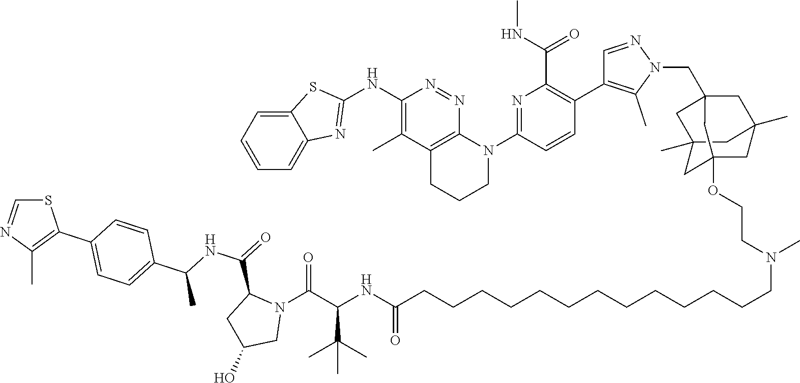 [SMILES: CNC(=O)c1nc(N2CCCc3c2nnc(Nc2nc4ccccc4s2)c3C)ccc1-c1cnn(CC23CC4(C)CC(C)(C2)CC(OCCN(C)CCCCCCCCCCCCCC(=O)N[C@H](C(=O)N2C[C@H](O)C[C@H]2C(=O)N[C@@H](C)c2ccc(-c5scnc5C)cc2)C(C)(C)C)(C4)C3)c1C]

To the mixture of 48 mg (0.034 mmol) of the product of Example 4 and 0.024 mL (5 eq) of triethylamine in 2 mL of DMF were added 16 mg (1.2 eq) of HATU. After 20 minutes, 0.034 mL (2 eq) of methylamine was also added. Then, the reaction was stirred for 2 h. After pouring the reaction into water, the precipitated solid was filtered out, washed with water, dried, and purified by column chromatography to give the desired compound (65%). HRMS-ESI (m/z): [M+2H]²⁺ calcd for C₇₉H₁₀₉N₁₄O₆S₂: 707.4082. found: 707.4090.

### Example 157: 6-[3-(1,3-benzothiazol-2-ylamino)-4-methyl-6,7-dihydro-5H-pyrido[2,3-c]pyridazin-8-yl]-3-[1-[[3-[2-[[14-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-14-oxo-tetradecyl]-methyl-amino]ethoxy]-5,7-dimethyl-1-adamantyl]methyl]-5-methyl-pyrazol-4-yl]-N-isopropyl-pyridine-2-carboxamide

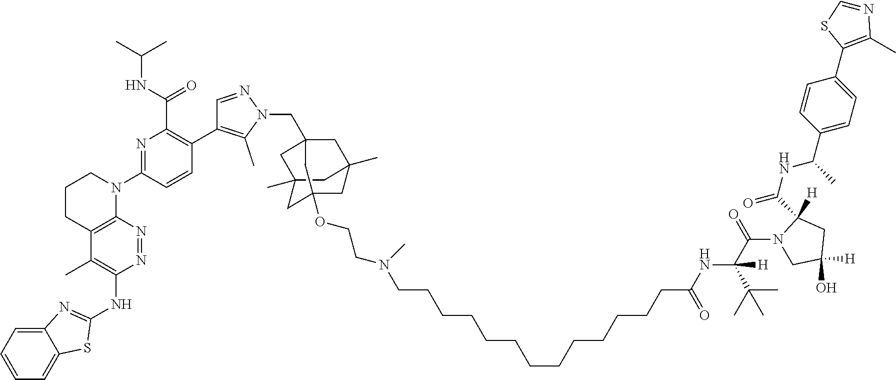 [SMILES: Cc1ncsc1-c1ccc([C@H](C)NC(=O)[C@@H]2C[C@@H](O)CN2C(=O)[C@@H](NC(=O)CCCCCCCCCCCCCN(C)CCOC23CC4(C)CC(C)(CC(Cn5ncc(-c6ccc(N7CCCc8c7nnc(Nc7nc9ccccc9s7)c8C)nc6C(=O)NC(C)C)c5C)(C4)C2)C3)C(C)(C)C)cc1]

To the mixture of 48 mg (0.034 mmol) of the product of Example 4 and 0.024 mL (5 eq) of triethylamine in 2 ml of DMF were added 16 mg (1.2 eq) of HATU. After 20 minutes, 0.006 mL (2 eq) of propan-2-amine was also added. Then, the reaction was stirred for 2 h. After pouring the reaction into water, the precipitated solid was filtered out, washed with water, dried, and purified by column chromatography to give the desired compound (71%). HRMS-ESI (m/z): [M+2H]²⁺ calcd for C₈₁H₁₁₄N₁₄O₆S₂: 721.4239. found: 721.4242.

### Example 158: 6-(3-(benzo[d]thiazol-2-ylamino)-4-methyl-6,7-dihydropyrido[2,3-c]pyridazin-8(5H)-yl)-3-(1-(((1r,3R,5S,7s)-3,5-dimethyl-7-(2-(pyrrolidin-1-yl)ethoxy)adamantan-1-yl)methyl)-5-methyl-1H-pyrazol-4-yl)-N-(2-(2-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropoxy)ethoxy)ethyl)picolinamide

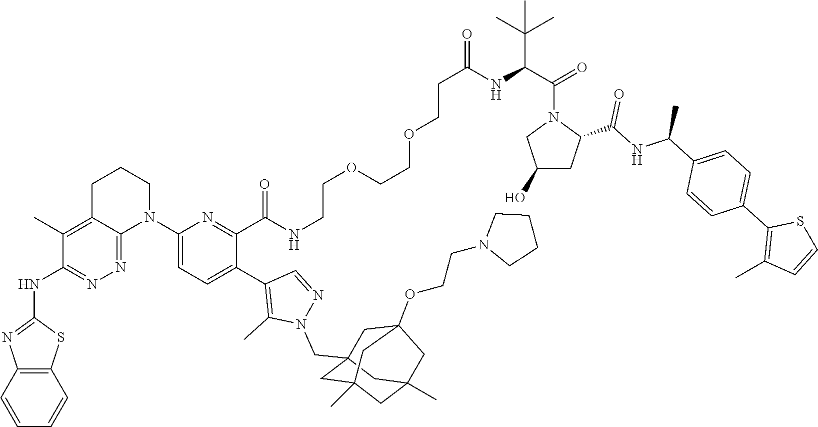 [SMILES: Cc1ccsc1-c1ccc([C@H](C)NC(=O)[C@@H]2C[C@@H](O)CN2C(=O)[C@@H](NC(=O)CCOCCOCCNC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C(C)(C)C)cc1]

A mixture of (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (8.9 mg, 0.020 mmol; see, e.g., Hu et al., J. Med. Chem., 2019, 62, 1420-1442 for preparation), 2,2-dimethyl-4-oxo-3,8,11-trioxa-5-azatetradecan-14-oic acid (6.1 mg, 0.022 mmol; available commercially), DIPEA (13 mg, 17 μL, 0.10 mmol), and HATU (8.4 mg, 0.022 mmol) in DMF (0.2 mL) was stirred at RT for 10 min. The mixture was concentrated, and the residue was taken up in DCM (1.0 mL) and TFA (500 μL, 6.490 mmol). The mixture was stirred at RT for 15 min. The mixture was concentrated. Ether was added to induce solid precipitation. Ether layer was removed and solid was dried under vacuum to give the crude product. This was taken up in DMF (0.2 mL). 6-(3-(benzo[d]thiazol-2-ylamino)-4-methyl-6,7-dihydropyrido[2,3-c]pyridazin-8(5H)-yl)-3-(1-(((1 r,3R,5S,7s)-3,5-dimethyl-7-(2-(pyrrolidin-1-yl)ethoxy)adamantan-1-yl)methyl)-5-methyl-1H-pyrazol-4-yl)picolinic acid (Preparation 21) trifluoroacetate salt (16 mg, 0.018 mmol), HATU (7.5 mg, 1.1 Eq, 20 μmol), and DIPEA (31 μL, 0.18 mmol) were added. The mixture was stirred at RT for 15 min. The mixture was concentrated, and the residue was purified by HPLC (MeCN-water, formic acid modifier) to give the title product as a yellow solid (7.3 mg). HRMS calc'd for C₇₄H₉₆N₁₄O₈S₂: 1372.6977. Found: 1373.8300 (M+1).

### Example 159: 6-(3-(benzo[d]thiazol-2-ylamino)-4-methyl-6,7-dihydropyrido[2,3-c]pyridazin-8(5H)-yl)-3-(1-(((1r,3R,5S,7s)-3,5-dimethyl-7-(2-(pyrrolidin-1-yl)ethoxy)adamantan-1-yl)methyl)-5-methyl-1H-pyrazol-4-yl)-N—((S)-14-((2S,4R)-4-hydroxy-2-(((S)-1-(4-(4-methyl thiazol-5-yl)phenyl)ethyl)carbamoyl)pyrrolidine-1-carbonyl)-15,15-dimethyl-12-oxo-3,6,9-trioxa-13-azahexadecyl)picolinamide

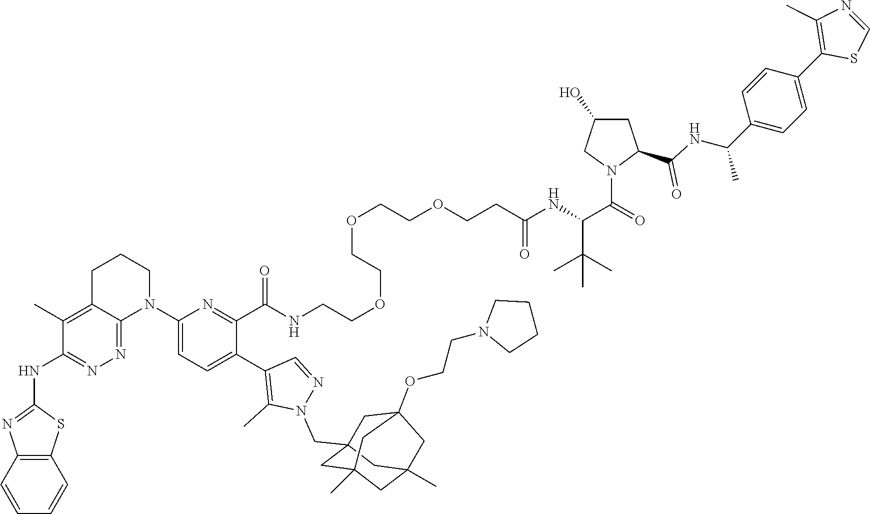 [SMILES: Cc1ncsc1-c1ccc([C@H](C)NC(=O)[C@@H]2C[C@@H](O)CN2C(=O)[C@@H](NC(=O)CCOCCOCCOCCNC(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C(C)(C)C)cc1]

The title compound was prepared in a similar manner to Example 158, using 2,2-dimethyl-4-oxo-3,8,11,14-tetraoxa-5-azaheptadecan-17-oic acid (available commercially) instead of 2,2-dimethyl-4-oxo-3,8,11-trioxa-5-azatetradecan-14-oic acid. HRMS calc'd for C₇₆H₁₀₀N₁₄09S₂: 1416.7239. Found: 1417.7200 (M+1).

### Example 160: 6-(3-(benzo[d]thiazol-2-ylamino)-4-methyl-6,7-dihydropyrido[2,3-c]pyridazin-8(5H)-yl)-3-(1-(((1r,3R,5S,7s)-3,5-dimethyl-7-(2-(pyrrolidin-1-yl)ethoxy)adamantan-1-yl)methyl)-5-methyl-1H-pyrazol-4-yl)-N—((S)-14-((2S,4R)-4-hydroxy-2-(((S)-1-(4-(4-methyl thiazol-5-yl)phenyl)ethyl)carbamoyl)pyrrolidine-1-carbonyl)-15,15-dimethyl-12-oxo-3,6,9-trioxa-13-azahexadecyl)-N-methylpicolinamide

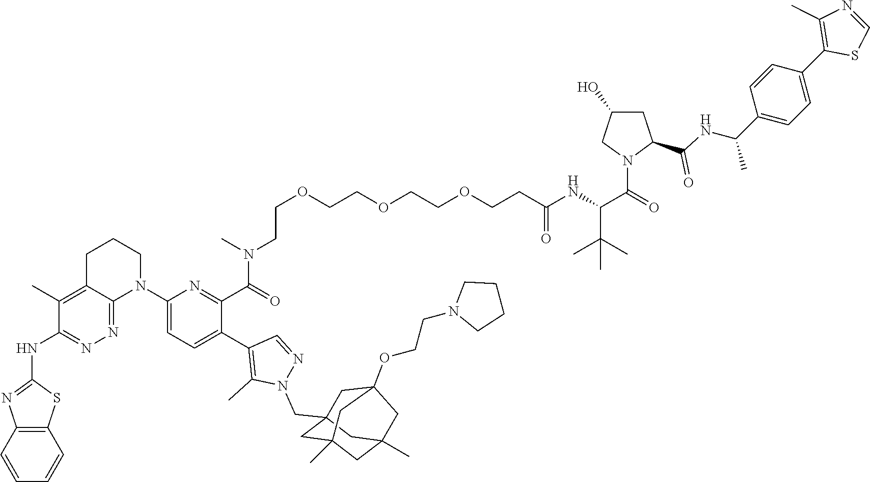 [SMILES: Cc1ncsc1-c1ccc([C@H](C)NC(=O)[C@@H]2C[C@@H](O)CN2C(=O)[C@@H](NC(=O)CCOCCOCCOCCN(C)C(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C(C)(C)C)cc1]

The title compound was prepared in a similar manner to Example 158, using 2,2,5-trimethyl-4-oxo-3,8,11,14-tetraoxa-5-azaheptadecan-17-oic acid (available commercially, e.g., from Princeton Biomolecular Research) instead of 2,2-dimethyl-4-oxo-3,8,11-trioxa-5-azatetradecan-14-oic acid. HRMS calc'd for C₇₇H₁₀₂N₁₄O₉S₂: 1430.7396. Found: 1431.7400 (M+1).

### Example 161: 6-(3-(benzo[d]thiazol-2-ylamino)-4-methyl-6,7-dihydropyrido[2,3-c]pyridazin-8(5H)-yl)-3-(1-(((1r,3R,5S,7s)-3,5-dimethyl-7-(2-(pyrrolidin-1-yl)ethoxy)adamantan-1-yl)methyl)-5-methyl-1H-pyrazol-4-yl)-N-(9-(((S)-1-((2S,4R)-4-hydroxy-2-(((S)-1-(4-(4-methyl thiazol-5-yl)phenyl)ethyl)carbamoyl)pyrrolidin-1-yl)-3,3-dimethyl-1-oxobutan-2-yl)amino)-9-oxononyl)-N-methylpicolinamide

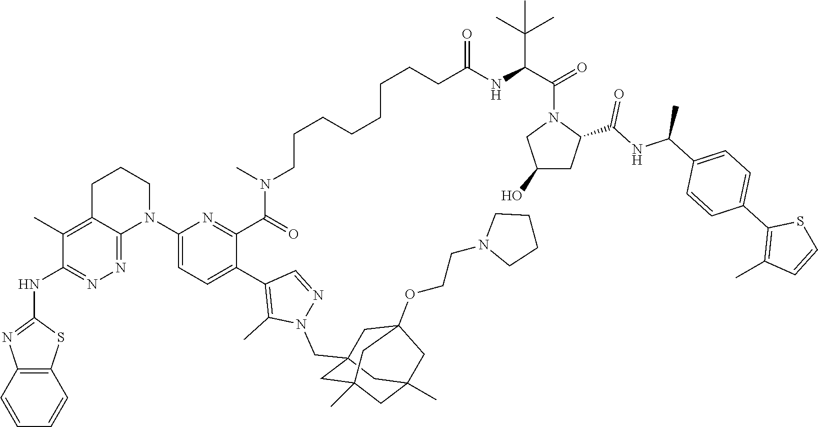 [SMILES: Cc1ccsc1-c1ccc([C@H](C)NC(=O)[C@@H]2C[C@@H](O)CN2C(=O)[C@@H](NC(=O)CCCCCCCCN(C)C(=O)c2nc(N3CCCc4c3nnc(Nc3nc5ccccc5s3)c4C)ccc2-c2cnn(CC34CC5(C)CC(C)(C3)CC(OCCN3CCCC3)(C5)C4)c2C)C(C)(C)C)cc1]

The title compound was prepared in a similar manner to Example 158, using tert-butyl methyl(9-methyldec-9-en-1-yl)carbamate instead of 2,2-dimethyl-4-oxo-3,8,11-trioxa-5-azatetradecan-14-oic acid. HRMS calc'd for C₇₇H₁₀₂N₁₄O₆S₂: 1382.7548. Found: 1383.8800 (M+1).

Preparation of tert-butyl methyl(9-methyldec-9-en-1-yl)carbamate: To a swirled solution of methylamine (26.7 g, 344 mmol) in water (30 mL) was added via syringe a solution of 9-bromononanoic acid (1.00 g, 4.22 mmol) in THF (10 mL). After addition, the homogeneous solution was capped and left standing at rt for 13.5 h. The volatiles were removed in vacuo with heat. The resulting oil was dissolved in MeOH (10 mL) and THF (10 mL), DIEA (0.737 mL, 4.22 mmol) was added followed by Boc₂O (2.76 g, 12.7 mmol). The solution was swirled, capped and left standing at rt for 2.5 h. The volatiles were removed in vacuo and the residue was purified by RP-column (MeCN/H₂O with 0.1% TFA as modifier) to give the title compound (802 mg) as a white solid after lyophilization. LCMS m/z 288.4 (MH+). 1H NMR (400 MHz, DMSO-d6) ppm 11.93 (s, 1H), 3.12 (t, J=7.2 Hz, 2H), 2.74 (s, 3H), 2.18 (t, J=7.4 Hz, 2H), 1.47 (qd, J=14.1, 13.6, 7.2 Hz, 4H), 1.38 (s, 9H), 1.30-1.13 (m, 8H).

### Example 162: 6-(3-(benzo[d]thiazol-2-ylamino)-4-methyl-6,7-dihydropyrido[2,3-c]pyridazin-8(5H)-yl)-3-(1-(((1r,3R,5S,7s)-3,5-dimethyl-7-(2-(pyrrolidin-1-yl)ethoxy)adamantan-1-yl)methyl)-5-methyl-1H-pyrazol-4-yl)-N-((1S,4R)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)cyclohexyl)picolinamide

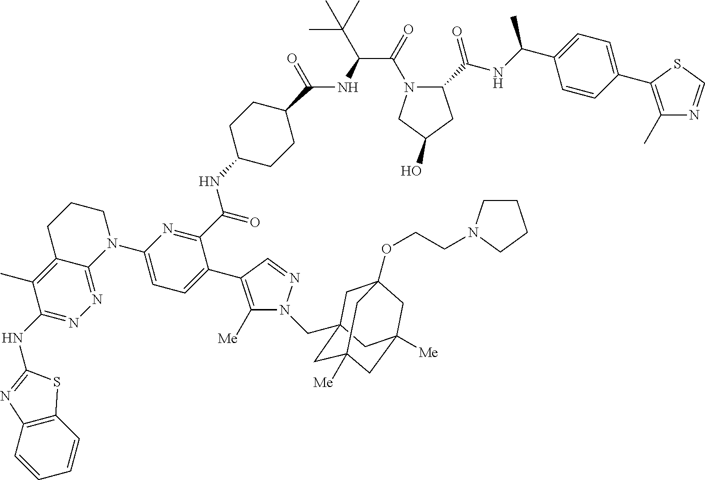 [SMILES: Cc1ncsc1-c1ccc([C@H](C)NC(=O)[C@@H]2C[C@@H](O)CN2C(=O)[C@@H](NC(=O)[C@H]2CC[C@H](NC(=O)c3nc(N4CCCc5c4nnc(Nc4nc6ccccc6s4)c5C)ccc3-c3cnn(CC45CC6(C)CC(C)(C4)CC(OCCN4CCCC4)(C6)C5)c3C)CC2)C(C)(C)C)cc1]

The title compound was prepared in a similar manner to Example 158, using (1 r,4r)-4-((tert-butoxycarbonyl)amino)cyclohexane-1-carboxylic acid (available commercially) instead of 2,2-dimethyl-4-oxo-3,8,11-trioxa-5-azatetradecan-14-oic acid. HRMS calc'd for C₇₄H₉₄N₁₄O₆S₂: 1338.6922. Found: 1339.7100 (M+1).

### Example 163: 6-(3-(benzo[d]thiazol-2-ylamino)-4-methyl-6,7-dihydropyrido[2,3-c]pyridazin-8(5H)-yl)-3-(1-(((1r,3R,5S,7s)-3,5-dimethyl-7-(2-(pyrrolidin-1-yl)ethoxy)adamantan-1-yl)methyl)-5-methyl-1H-pyrazol-4-yl)-N-((1R,4S)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)picolinamide

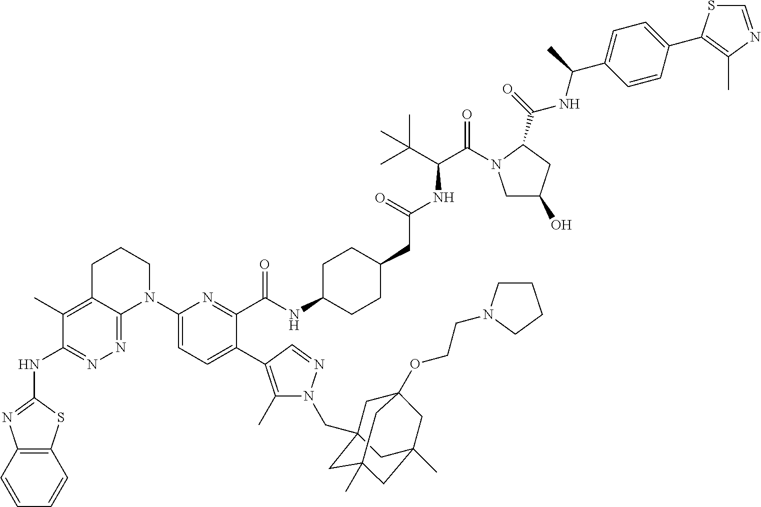 [SMILES: Cc1ncsc1-c1ccc([C@H](C)NC(=O)[C@@H]2C[C@@H](O)CN2C(=O)[C@@H](NC(=O)C[C@H]2CC[C@@H](NC(=O)c3nc(N4CCCc5c4nnc(Nc4nc6ccccc6s4)c5C)ccc3-c3cnn(CC45CC6(C)CC(C)(C4)CC(OCCN4CCCC4)(C6)C5)c3C)CC2)C(C)(C)C)cc1]

The title compound was prepared in a similar manner to Example 158, using 2-((1s,4s)-4-((tert-butoxycarbonyl)amino)cyclohexyl)acetic acid (available commercially) instead of 2,2-dimethyl-4-oxo-3,8,11-trioxa-5-azatetradecan-14-oic acid. HRMS calc'd for C₇₅H₉₆N₁₄O₆S₂: 1352.7079. Found: 1353.7200 (M+1).

### Example 164: 6-(3-(benzo[d]thiazol-2-ylamino)-4-methyl-6,7-dihydropyrido[2,3-c]pyridazin-8(5H)-yl)-3-(1-(((1r,3R,5S,7s)-3,5-dimethyl-7-(2-(pyrrolidin-1-yl)ethoxy)adamantan-1-yl)methyl)-5-methyl-1H-pyrazol-4-yl)-N-((1S,4R)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)picolinamide

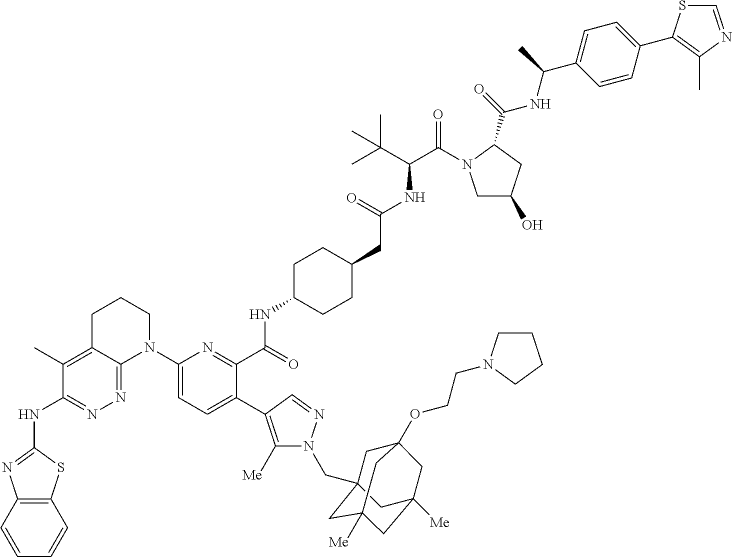 [SMILES: Cc1ncsc1-c1ccc([C@H](C)NC(=O)[C@@H]2C[C@@H](O)CN2C(=O)[C@@H](NC(=O)C[C@H]2CC[C@H](NC(=O)c3nc(N4CCCc5c4nnc(Nc4nc6ccccc6s4)c5C)ccc3-c3cnn(CC45CC6(C)CC(C)(C4)CC(OCCN4CCCC4)(C6)C5)c3C)CC2)C(C)(C)C)cc1]

The title compound was prepared in a similar manner to Example 158, using 2-((1 r,4r)-4-((tert-butoxycarbonyl)amino)cyclohexyl)acetic acid (available commercially) instead of 2,2-dimethyl-4-oxo-3,8,11-trioxa-5-azatetradecan-14-oic acid. HRMS calc'd for C₇₅H₉₆N₁₄O₆S₂: 1352.7079. Found: 1353.7200 (M+1).

### Example 165: 6-(3-(benzo[d]thiazol-2-ylamino)-4-methyl-6,7-dihydropyrido[2,3-c]pyridazin-8(5H)-yl)-3-(1-(((1r,3R,5S,7s)-3,5-dimethyl-7-(2-(pyrrolidin-1-yl)ethoxy)adamantan-1-yl)methyl)-5-methyl-1H-pyrazol-4-yl)-N-((1S,3R)-3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)cyclobutyl)picolinamide

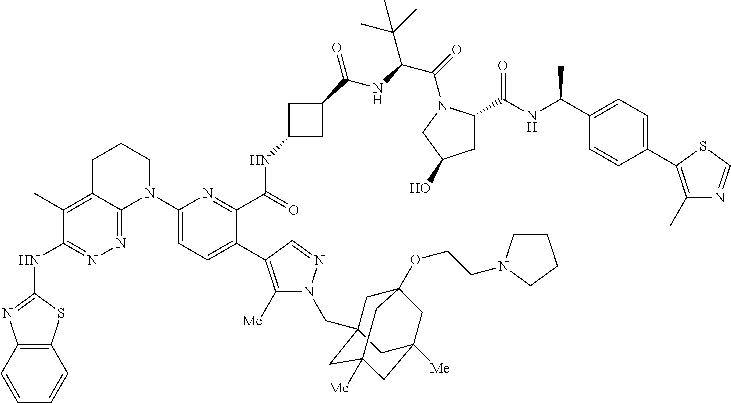 [SMILES: Cc1ncsc1-c1ccc([C@H](C)NC(=O)[C@@H]2C[C@@H](O)CN2C(=O)[C@@H](NC(=O)[C@H]2C[C@H](NC(=O)c3nc(N4CCCc5c4nnc(Nc4nc6ccccc6s4)c5C)ccc3-c3cnn(CC45CC6(C)CC(C)(C4)CC(OCCN4CCCC4)(C6)C5)c3C)C2)C(C)(C)C)cc1]

The title compound was prepared in a similar manner to Example 158, using (1 r,3r)-3-((tert-butoxycarbonyl)amino)cyclobutane-1-carboxylic acid (available commercially) instead of 2,2-dimethyl-4-oxo-3,8,11-trioxa-5-azatetradecan-14-oic acid. HRMS calc'd for C₇₂H₉₀N₁₄O₆S₂: 1310.6609. Found: 1311.6801 (M+1).

### Example 166: (2S,4R)-1-((S)-2-(2-((1-(6-(3-(benzo[d]thiazol-2-ylamino)-4-methyl-6,7-dihydropyrido[2,3-c]pyridazin-8(5H)-yl)-3-(1-(((1r,3R,5S,7s)-3,5-dimethyl-7-(2-(pyrrolidin-1-yl)ethoxy)adamantan-1-yl)methyl)-5-methyl-1H-pyrazol-4-yl)picolinoyl)piperidin-4-yl)oxy) acetamido)-3,3-dimethylbutanoyl)-4-hydroxy-N—((S)-1-(4-(4-methylthiazol-5-yl)phenyl)ethyl)pyrrolidine-2-carboxamide

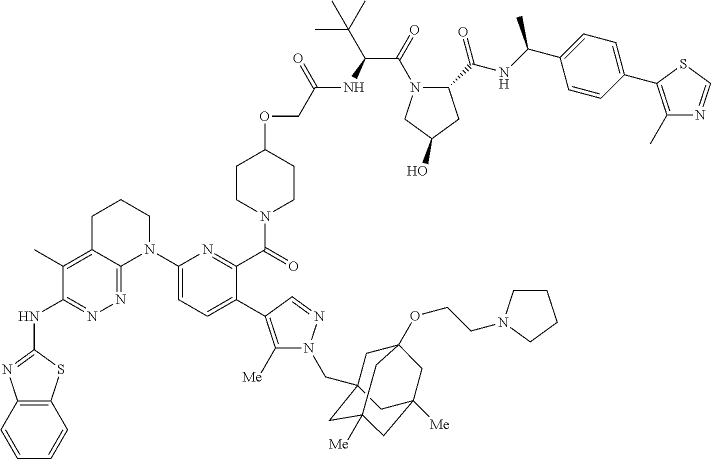 [SMILES: Cc1ncsc1-c1ccc([C@H](C)NC(=O)[C@@H]2C[C@@H](O)CN2C(=O)[C@@H](NC(=O)COC2CCN(C(=O)c3nc(N4CCCc5c4nnc(Nc4nc6ccccc6s4)c5C)ccc3-c3cnn(CC45CC6(C)CC(C)(C4)CC(OCCN4CCCC4)(C6)C5)c3C)CC2)C(C)(C)C)cc1]

The title compound was prepared in a similar manner to Example 158, using 2-((1-(tert-butoxycarbonyl)piperidin-4-yl)oxy)acetic acid (available commercially) instead of 2,2-dimethyl-4-oxo-3,8,11-trioxa-5-azatetradecan-14-oic acid. HRMS calc'd for C₇₄H₉₄N₁₄O₇S₂: 1354.6871. Found: 1355.7000 (M+1).

### Example 167: (2S,4R)-1-((S)-2-(3-((1-(6-(3-(benzo[d]thiazol-2-ylamino)-4-methyl-6,7-dihydropyrido[2,3-c]pyridazin-8(5H)-yl)-3-(1-(((1r,3R,5S,7s)-3,5-dimethyl-7-(2-(pyrrolidin-1-yl)ethoxy)adamantan-1-yl)methyl)-5-methyl-1H-pyrazol-4-yl)picolinoyl)piperidin-4-yl)oxy)propanamido)-3,3-dimethylbutanoyl)-4-hydroxy-N—((S)-1-(4-(4-methylthiazol-5-yl)phenyl)ethyl)pyrrolidine-2-carboxamide

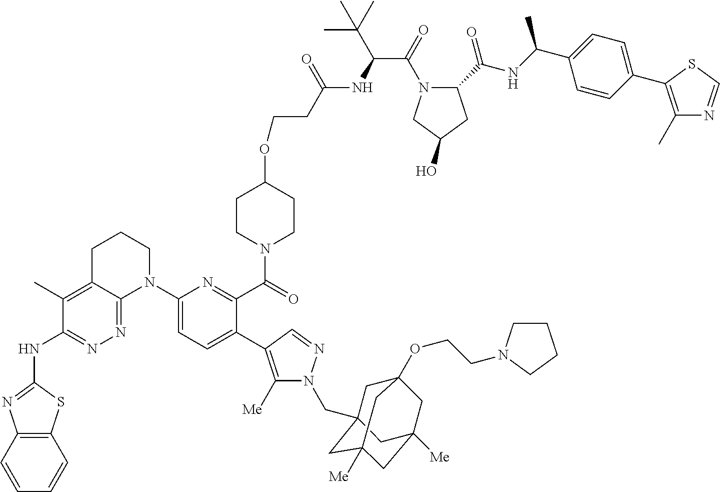 [SMILES: Cc1ncsc1-c1ccc([C@H](C)NC(=O)[C@@H]2C[C@@H](O)CN2C(=O)[C@@H](NC(=O)CCOC2CCN(C(=O)c3nc(N4CCCc5c4nnc(Nc4nc6ccccc6s4)c5C)ccc3-c3cnn(CC45CC6(C)CC(C)(C4)CC(OCCN4CCCC4)(C6)C5)c3C)CC2)C(C)(C)C)cc1]

The title compound was prepared in a similar manner to Example 158, using 3-((1-(tert-butoxycarbonyl)piperidin-4-yl)oxy)propanoic acid (available commercially) instead of 2,2-dimethyl-4-oxo-3,8,11-trioxa-5-azatetradecan-14-oic acid. HRMS calc'd for C₇₅H₉₆N₁₄O₇S₂: 1368.7028. Found: 1369.7200 (M+1).

### Example 168: (S)-1-(6-(3-(benzo[d]thiazol-2-ylamino)-4-methyl-6,7-dihydropyrido[2,3-c]pyridazin-8(5H)-yl)-3-(1-(((1r,3R,5S,7R)-3,5-dimethyl-7-(2-(pyrrolidin-1-yl)ethoxy) adamantan-1-yl)methyl)-5-methyl-1H-pyrazol-4-yl)picolinoyl)-N—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)piperidine-3-carboxamide

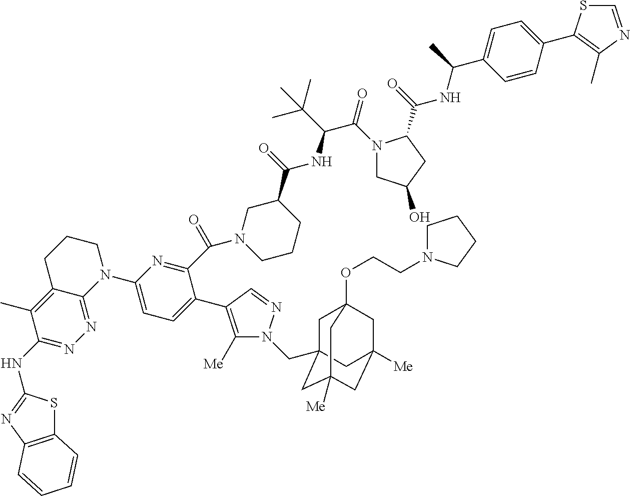 [SMILES: Cc1ncsc1-c1ccc([C@H](C)NC(=O)[C@@H]2C[C@@H](O)CN2C(=O)[C@@H](NC(=O)[C@H]2CCCN(C(=O)c3nc(N4CCCc5c4nnc(Nc4nc6ccccc6s4)c5C)ccc3-c3cnn(CC45CC6(C)CC(C)(C4)CC(OCCN4CCCC4)(C6)C5)c3C)C2)C(C)(C)C)cc1]

The title compound was prepared in a similar manner to Example 158, using (S)-1-(tert-butoxycarbonyl)piperidine-3-carboxylic acid (available commercially) instead of 2,2-dimethyl-4-oxo-3,8,11-trioxa-5-azatetradecan-14-oic acid. HRMS calc'd for C₇₃H₉₂N₁₄O₆S₂: 1324.6766. Found: 1325.6801 (M+1).

### Example 169: (R)-1-(6-(3-(benzo[d]thiazol-2-ylamino)-4-methyl-6,7-dihydropyrido[2,3-c]pyridazin-8(5H)-yl)-3-(1-(((1r,3R,5S,7S)-3,5-dimethyl-7-(2-(pyrrolidin-1-yl)ethoxy) adamantan-1-yl)methyl)-5-methyl-1H-pyrazol-4-yl)picolinoyl)-N—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)piperidine-3-carboxamide

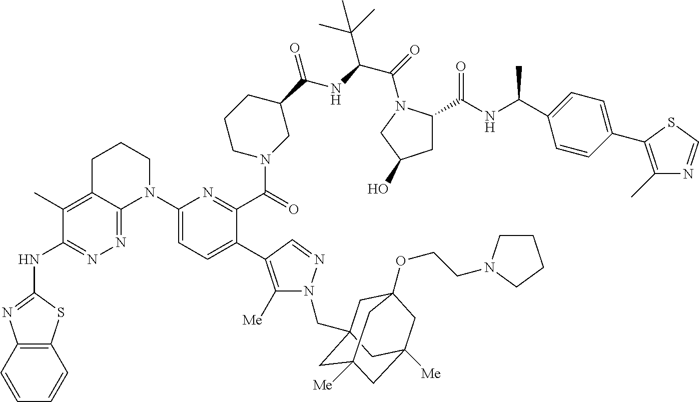 [SMILES: Cc1ncsc1-c1ccc([C@H](C)NC(=O)[C@@H]2C[C@@H](O)CN2C(=O)[C@@H](NC(=O)[C@@H]2CCCN(C(=O)c3nc(N4CCCc5c4nnc(Nc4nc6ccccc6s4)c5C)ccc3-c3cnn(CC45CC6(C)CC(C)(C4)CC(OCCN4CCCC4)(C6)C5)c3C)C2)C(C)(C)C)cc1]

The title compound was prepared in a similar manner to Example 158, using (R)-1-(tert-butoxycarbonyl)piperidine-3-carboxylic acid (available commercially) instead of 2,2-dimethyl-4-oxo-3,8,11-trioxa-5-azatetradecan-14-oic acid. HRMS calc'd for C₇₃H₉₂N₁₄O₆S₂: 1324.6766. Found: 1325.7000 (M+1).

### Example 170: (2S,4R)-1-((S)-2-(2-(2-(6-(3-(benzo[d]thiazol-2-ylamino)-4-methyl-6,7-dihydro pyrido[2,3-c]pyridazin-8(5H)-yl)-3-(1-(((1r,3R,5S,7s)-3,5-dimethyl-7-(2-(pyrrolidin-1-yl)ethoxy)adamantan-1-yl)methyl)-5-methyl-1H-pyrazol-4-yl)picolinoyl)-2-azaspiro [3.3]heptan-6-yl)acetamido)-3,3-dimethylbutanoyl)-4-hydroxy-N—((S)-1-(4-(4-methylthiazol-5-yl)phenyl)ethyl)pyrrolidine-2-carboxamide

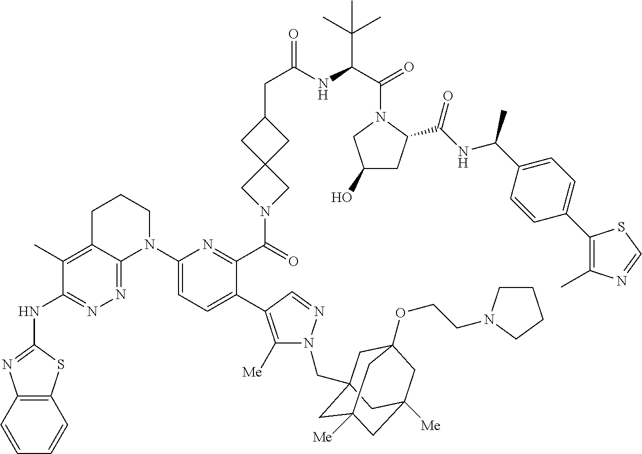 [SMILES: Cc1ncsc1-c1ccc([C@H](C)NC(=O)[C@@H]2C[C@@H](O)CN2C(=O)[C@@H](NC(=O)CC2CC3(C2)CN(C(=O)c2nc(N4CCCc5c4nnc(Nc4nc6ccccc6s4)c5C)ccc2-c2cnn(CC45CC6(C)CC(C)(C4)CC(OCCN4CCCC4)(C6)C5)c2C)C3)C(C)(C)C)cc1]

The title compound was prepared in a similar manner to Example 158, using 2-(2-(tert-butoxycarbonyl)-2-azaspiro[3.3]heptan-6-yl)acetic acid (available commercially) instead of 2,2-dimethyl-4-oxo-3,8,11-trioxa-5-azatetradecan-14-oic acid. HRMS calc'd for C₇₅H₉₄N₁₄O₆S₂: 1350.6922. Found: 1351.7100 (M+1).

### Example 171: 6-(3-(benzo[d]thiazol-2-ylamino)-4-methyl-6,7-dihydropyrido[2,3-c]pyridazin-8(5H)-yl)-3-(1-(((1r,3R,5S,7S)-3,5-dimethyl-7-(2-(pyrrolidin-1-yl)ethoxy)adamantan-1-yl)methyl)-5-methyl-1H-pyrazol-4-yl)-N—((R)-1-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutanoyl)piperidin-3-yl)picolinamide)

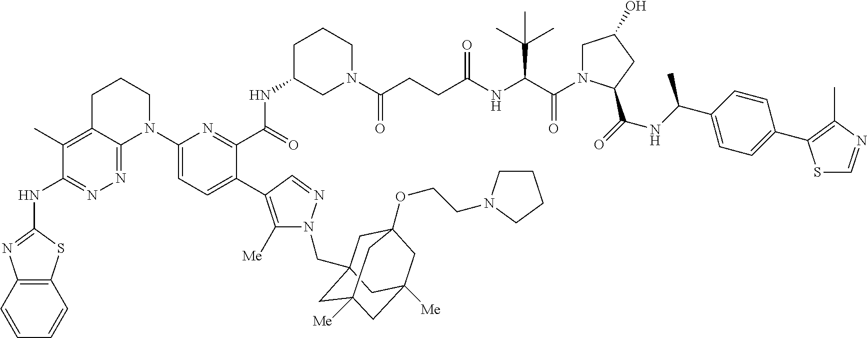 [SMILES: Cc1ncsc1-c1ccc([C@H](C)NC(=O)[C@@H]2C[C@@H](O)CN2C(=O)[C@@H](NC(=O)CCC(=O)N2CCC[C@@H](NC(=O)c3nc(N4CCCc5c4nnc(Nc4nc6ccccc6s4)c5C)ccc3-c3cnn(CC45CC6(C)CC(C)(C4)CC(OCCN4CCCC4)(C6)C5)c3C)C2)C(C)(C)C)cc1]

The reaction mixture then was diluted with DMSO and purified through RFC (MeCN/Water, 10-100%, 0.1% TFA) to give tert-butyl ((R)-1-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutanoyl)piperidin-3-yl)carbamate as a white solid (67 mg, 100% yield, MS m/z=727.5 [M+H]⁺).

The above product was taken up in DCM (1.0 mL) and TFA (250 μL). The mixture was stirred at RT for 1 h. The mixture was concentrated, diluted with DMSO and purified through RFC (MeCN/Water, 10-100%, 0.1% TFA) to give (2S,4R)-1-((S)-2-(4-((R)-3-aminopiperidin-1-yl)-4-oxobutanamido)-3,3-dimethylbutanoyl)-4-hydroxy-N—((S)-1-(4-(4-methylthiazol-5-yl)phenyl)ethyl)pyrrolidine-2-carboxamide trifluoroacetate salt as a white solid (53 mg, 78% yield, MS m/z=627.4 [M+H]⁺).

A mixture of of above product (2S,4R)-1-((S)-2-(4-((R)-3-aminopiperidin-1-yl)-4-oxobutanamido)-3,3-dimethylbutanoyl)-4-hydroxy-N—((S)-1-(4-(4-methylthiazol-5-yl)phenyl)ethyl)pyrrolidine-2-carboxamide trifluoroacetate salt (18 mg, 24 μmol), 6-(3-(benzo[d]thiazol-2-ylamino)-4-methyl-6,7-dihydropyrido[2,3-c]pyridazin-8(5H)-yl)-3-(1-(((1r,3R,5S,7s)-3,5-dimethyl-7-(2-(pyrrolidin-1-yl)ethoxy)adamantan-1-yl)methyl)-5-methyl-1H-pyrazol-4-yl)picolinic acid (Preparation 21) trifluoroacetate salt (18 mg, 0.020 mmol), HATU (11 mg, 1.5 Eq, 30 μmol) and DIPEA (13 mg, 17 μL, 5 Eq, 0.10 mmol) in DMF (0.5 mL). The reaction mixture was stirred at rt for 2 h. The RXN mixture then was diluted with DMSO and purified through RFC (MeCN/Water, 10-100%, 0.1% TFA) to give the title compound as a yellow solid. HRMS calc'd for C₇₆H₉₇N₁₅O₇S₂: 1395.7137. Found: 1396.7300 (M+1).

### Example 172: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-N-[7-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-7-oxo-heptyl]sulfonyl-pyridine-2-carboxamide

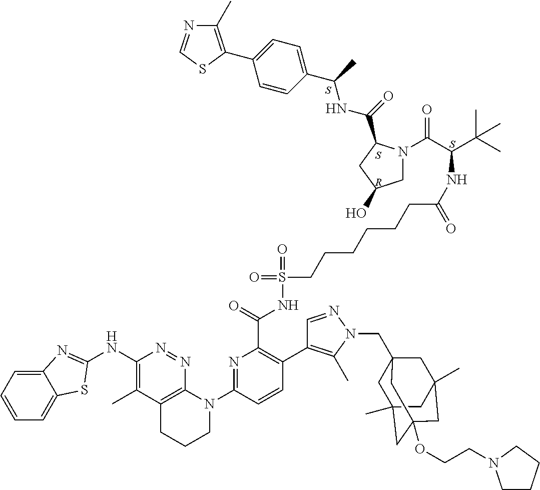

### Step A: tert-butyl 7-[[6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]pyridine-2-carbonyl]sulfamoyl]heptanoate

The mixture of Preparation 21 (1 eq), tert-butyl 7-sulfamoylheptanoate (2 eq), N,N-dimethylpyridin-4-amine (1.7 eq), and 3-(ethyliminomethyleneamino)-N,N-dimethyl-propan-1-amine hydrochloride (2 eq) in dichloromethane (20 mL/mmol) is stirred for 18 h. The product is purified via column chromatography to give the desired product.

### Step B: 7-[[6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]pyridine-2-carbonyl]sulfamoyl]heptanoic acid

After the treatment of the product of Step A with 2,2,2-trifluoroacetic acid (125 eq) in dichloromethane (10 mL/mmol), the reaction is stirred to reach the appropriate conversion. The product is purified by preparative HPLC to give the desired product.

### Step C: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-N-[7-[[(1S)-1-[(2S,4R)-4-hydroxy-2-[[(1S)-1-[4-(4-methylthiazol-5-yl)phenyl]ethyl] carbamoyl]pyrrolidine-1-carbonyl]-2,2-dimethyl-propyl]amino]-7-oxo-heptyl]sulfonyl-pyridine-2-carboxamide

Using the General procedure for the acylation of VHL ligands starting from (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide, hydrogen chloride (1:1) (0.42 mmmol) and the product of Step B as the appropriate acid, the desired product is obtained.

### Example 173: 6-[3-(1,3-benzothiazol-2-ylamino)-4-methyl-6,7-dihydro-5H-pyrido[2,3-c]pyridazin-8-yl]-3-[1-[[3-[2-[2-[2-[3-[[(1S)-1-[(2S,4R)-4-hydroxy-2-[[(1S)-1-[4-(4-methyl thiazol-5-yl)phenyl]ethyl]carbamoyl]pyrrolidine-1-carbonyl]-2,2-dimethyl-propyl]amino]-3-oxo-propoxy]ethoxy]ethyl-methyl-amino]ethoxy]-5,7-dimethyl-1-adamantyl]methyl]-5-methyl-pyrazol-4-yl]pyridine-2-carboxylic acid

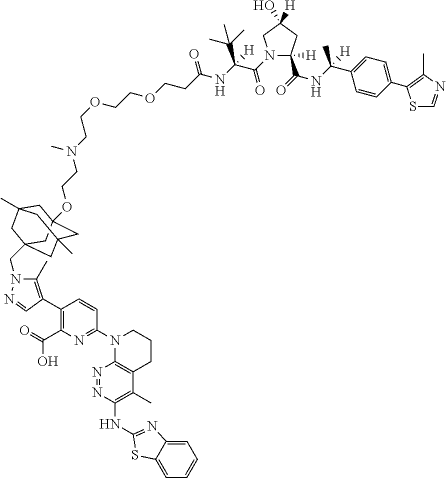 [SMILES: Cc1ncsc1-c1ccc([C@H](C)NC(=O)[C@@H]2C[C@@H](O)CN2C(=O)[C@@H](NC(=O)CCOCCOCCN(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)C(C)(C)C)cc1]

### Step A: tert-butyl 3-[2-[2-(p-tolylsulfonyloxy)ethoxy]ethoxy]propanoate

To the mixture of tert-butyl 3-[2-(2-hydroxyethoxy)ethoxy]propanoate (1.5 g) and TEA (1.7 mL) in DCM (32 mL) was added 4-methylbenzenesulfonyl chloride (1.4 g) at 0° C. Then, the reaction was stirred for 1 h. After quenching the reaction with aqueous NH₄Cl and extracting with DCM, the product was purified by column chromatography to give 1.67 g of the desired product. ¹H NMR (500 MHz, DMSO-d₆): δ ppm 7.79 (d, 2H), 7.49 (d, 2H), 4.10 (t, 2H), 3.57 (t, 2H), 3.55 (t, 2H), 3.43 (m, 2H), 3.43 (m, 2H), 2.43 (s, 3H), 2.40 (t, 2H), 1.39 (s, 9H); ¹³C NMR (125 MHz, DMSO-d₆) δ ppm 170.9, 145.4, 132.9, 130.6, 128.1, 80.2, 70.4, 70.1, 70.0, 68.3, 66.7, 36.3, 28.2, 21.6; HRMS-ESI (m/z): [M+Na]⁺ calcd for C₁₈H₂₈NaO₇S: 411.1448. found: 411.1452.

### Step B: 3-[2-[2-(p-tolylsulfonyloxy)ethoxy]ethoxy]propanoic acid

After stirring the product of Step A (1.0 g) and TFA (1.5 mL) in DCM (13 mL) for 18 h, the volatiles were removed under reduced pressure to give 832 mg of the desired product. ¹H NMR (500 MHz, DMSO-d₆): δ ppm 11.88 (brs, 1H), 7.79 (d, 2H), 7.49 (d, 2H), 4.11 (t, 2H), 3.57 (t, 2H), 3.57 (t, 2H), 3.43 (m, 2H), 3.43 (m, 2H), 2.43 (s, 3H), 2.43 (t, 2H); ¹³C NMR (125 MHz, DMSO-d₆) δ ppm 173.1, 145.4, 132.9, 130.6, 128.1, 70.5, 70.0, 69.9, 68.3, 66.7, 35.2, 21.6; HRMS-ESI (m/z): [M+Na]⁺ calcd for C₁₄H₂₀NaO₇S: 355.0822. found: 355.0822.

### Step C: 2-[2-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]ethoxy]ethyl 4-methylbenzenesulfonate

Using General procedure for the acylation of VHL ligands starting from 300 mg of (2S,4R)-1-[(2S)-2-amino-3,3-dimethyl-butanoyl]-4-hydroxy-N-[(1S)-1-[4-(4-methylthiazol-5-yl)phenyl]ethyl] pyrrolidine-2-carboxamide, hydrogen chloride (1:1) (0.62 mmol) and the product of Step B as the appropriate acid, 457 mg of the desired product were obtained. ¹H NMR (500 MHz, DMSO-d₆): δ ppm 8.98 (s, 1H), 8.38 (d, 1H), 7.86 (d, 1H), 7.79 (dm, 2H), 7.48 (dm, 2H), 7.44 (dm, 2H), 7.38 (dm, 2H), 5.11 (br., 1H), 4.91 (m, 1H), 4.52 (d, 1H), 4.42 (t, 1H), 4.27 (br., 1H), 4.10 (m, 2H), 3.65-3.36 (m, 10H), 2.51/2.33 (m+m, 2H), 2.45 (s, 3H), 2.42 (s, 3H), 2.01/1.78 (m+m, 2H), 1.37 (d, 3H), 0.92 (s, 9H); ¹³C NMR (125 MHz, DMSO-d₆) δ ppm 171.1, 170.3, 169.9, 150.6, 130.6, 129.3, 128.1, 126.9, 70.5, 69.2, 59.0, 56.8, 48.2, 38.2, 36.1, 26.9, 22.9, 21.6, 16.5; HRMS-ESI (m/z): [M+H]⁺ calcd for C₃₇H₅₁N₄O₉S₂: 759.3092. found: 759.3093.

### Step D: 6-[3-(1,3-benzothiazol-2-ylamino)-4-methyl-6,7-dihydro-5H-pyrido[2,3-c]pyridazin-8-yl]-3-[1-[[3-[2-[2-[2-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]ethoxy]ethyl-methyl-amino]ethoxy]-5,7-dimethyl-1-adamantyl]methyl]-5-methyl-pyrazol-4-yl]pyridine-2-carboxylic acid

Using Degrader Synthesis by Alkylation and Hydrolysis General Procedure starting from the product of Step C as the appropriate alkylating agent and 30 mg of Preparation 4 as the appropriate amine, 12 mg of the desired product were obtained. HRMS-ESI (m/z): [M+H]⁺ calcd for C₇₁H₉₂N₁₃O₉S₂: 1334.6577. found: 1334.6592.

### Example 174: 2-[3-(1,3-benzothiazol-2-ylamino)-4-methyl-6,7-dihydro-5H-pyrido[2,3-c]pyridazin-8-yl]-5-[3-[4-[3-[4-[5-[2-[2-[[2-[2-(2,6-dioxo-3-piperidyl)-1,3-dioxo-isoindolin-4-yl]oxyacetyl]amino]ethoxy]ethylamino]-5-oxo-pentanoyl]piperazin-1-yl]prop-1-ynyl]-2-fluoro-phenoxy]propyl]thiazole-4-carboxylic acid

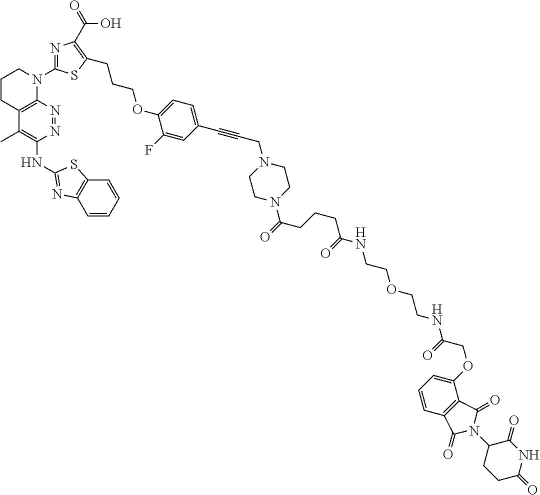 [SMILES: Cc1c(Nc2nc3ccccc3s2)nnc2c1CCCN2c1nc(C(=O)O)c(CCCOc2ccc(C#CCN3CCN(C(=O)CCCC(=O)NCCOCCNC(=O)COc4cccc5c4C(=O)N(C4CCC(=O)NC4=O)C5=O)CC3)cc2F)s1]

### Step A: 5-[2-[2-[[2-[2-(2,6-dioxo-3-piperidyl)-1,3-dioxo-isoindolin-4-yl]oxyacetyl]amino]ethoxy]ethylamino]-5-oxo-pentanoic acid

The mixture of 5-[2-[2-[[2-[2-(2,6-dioxo-3-piperidyl)-1,3-dioxo-isoindolin-4-yl]oxyacetyl]amino]ethoxy]ethylamino]-5-oxo-pentanoic acid (200 mg), tetrahydropyran-2,6-dione (65 mg), and TEA (0.20 mL) in DCM (2.4 mL) was stirred for 18 h. Purification of the product by preparative HPLC led to 154 mg of the desired product. ¹H NMR (500 MHz, DMSO-d₆): δ ppm 12.02 (br., 1H), 11.12 (s, 1H), 8.02 (t, 1H), 7.83 (t, 1H), 7.81 (dd, 1H), 7.49 (d, 1H), 7.39 (d, 1H), 5.12 (dd, 1H), 4.79 (s, 2H), 3.45 (t, 2H), 3.40 (t, 2H), 3.31 (q, 2H), 3.20 (q, 2H), 2.90/2.59 (ddd+dm, 2H), 2.53/2.04 (m+m, 2H), 2.18 (t, 2H), 2.08 (t, 2H), 1.68 (quint., 2H); ¹³C NMR (125 MHz, DMSO-d₆) δ ppm 174.7, 173.3/170.4, 172.2, 167.4, 167.2/165.9, 137.4, 120.8, 116.5, 69.4, 69.0, 67.9, 49.3, 38.8, 38.8, 34.8, 33.4, 31.4, 22.5, 21.1; HRMS-ESI (m/z): [M+H]⁺ calcd for C₂₄H₂₉N₄O₁₀: 533.1880. found: 533.1878.

### Step B: 2-[3-(1,3-benzothiazol-2-ylamino)-4-methyl-6,7-dihydro-5H-pyrido[2,3-c]pyridazin-8-yl]-5-[3-[4-[3-[4-[5-[2-[2-[[2-[2-(2,6-dioxo-3-piperidyl)-1,3-dioxo-isoindolin-4-yl]oxyacetyl]amino]ethoxy]ethylamino]-5-oxo-pentanoyl]piperazin-1-yl]prop-1-ynyl]-2-fluoro-phenoxy]propyl]thiazole-4-carboxylic acid

After mixing the product of Step A (57 mg), HATU (46 mg), and TEA (0.10 mL) in DMF (0.72 mL) at 50° C. for 5 min, Preparation 9 (50 mg) was added. Then, the reaction was stirred for 1 h. Purification by preparative HPLC afforded 18 mg of the desired product. HRMS-ESI (m/z): [M+H]⁺ calcd for C₅₉H₆₂FN₁₂O₁₂S₂: 1213.4030. found: 1213.4031.

### D. In Vitro Assessment of Bcl-xL Degrader Compounds Against the Endogenous Cancer Cell Lines: COR-L105 & NCI-H1650

### Cell Lines

The BCL-xL degrader compounds were tested against two endogenous cancer cell lines:
COR-L105: ECACC No. 92031918 cultured in RPMI-1640+10% FBS
NCI-H1650: (ATCC No. CRL-5883 cultured in RPMI-1640+10% FBS

### Inhibition of Cell Proliferation and Survival

The ability of the BCL-xL degrader low molecular weight compounds to inhibit cell proliferation and survival was assessed using the Promega CellTiter-Glo© proliferation assay.

Cell lines were cultured in media that is optimal for their growth at 5% CO₂, 37° C. in a tissue culture incubator. Prior to seeding for the proliferation assay, the cells were split at least 2 days before the assay to ensure optimal growth density. On the day of seeding, adherent cells were lifted off tissue culture flasks using 0.25% trypsin. Cell viability and cell density were determined using a cell counter (Vi-Cell XR Cell Viability Analyzer, Beckman Coulter). Cells with higher than 85% viability were seeded for the assay.

The COR-105 cell line was seeded in white, clear bottom 384-well TC treated plates (Corning cat. #3765). Cells were seeded at a density of 1,000 cells per well in 45 μL of standard growth media. Plates were incubated at 5% CO₂, 37° C. overnight in a tissue culture incubator. The NCI-H1650 cell line was seeded in black, clear round bottom 384-well ultra-low attachment spheroid microplates (Corning cat. #3830). Cells were seeded at a density of 3,000 cells per weill in 45 uL of standard growth media. Plates were spun in a centrifuge for 5 minutes and 1,000 RPM. Plates were incubated at 5% CO₂, 37° C. for 72 hours in a tissue culture incubator.

On the day of dosing, BCL-xL degrader low molecular weight compounds were prepared at 10× in standard growth media. The prepared drug treatments were then added to the cells resulting in final concentrations of 0.508 nM-10 μM and a final volume of 50 μL per well. Each drug concentration was tested in quadruplets. Plates were incubated at 5% CO₂, 37° C. for 5 days in a tissue culture incubator.

For COR-L105 plates, cell viability was assessed through the addition of 25 μL of CellTiter Glo® (Promega, cat #G7573), a reagent which lyses cells and measures total adenosine triphosphate (ATP) content. Plates were incubated at room temperature for 10 minutes to stabilize luminescent signals prior to reading with a luminescence reader (EnVision Multilabel Plate Reader, PerkinElmer). For NCI-H1650 plates, cell viability was assessed through the addition of 40 μL of CellTiter Glo® 3D Cell Viability Assay substrate (Promega, cat #G9681), a reagent which lyses cells and measures total adenosine triphosphate (ATP) content. Wells were mixed thoroughly and plates were incubated at room temperature for 30 minutes to stabilize luminescent signals prior to reading using a luminescence reader (EnVision Multilabel Plate Reader, PerkinElmer).

To evaluate the effect of the drug treatments, luminescent counts from wells containing untreated cells (100% viability) were used to normalize treated samples. A variable slope model was applied to fit a nonlinear regression curve to the data in GraphPad PRISM version 7.02 software. IC50 and Amax values were extrapolated from the resultant curves. The concentrations of treatment required to inhibit 50% of cell growth or survival (IC50) were calculated with representative IC50 values of the cell lines tested summarized in Table 11.

The representative cancer cell lines were shown to be sensitive to BCL-xL degrader low molecular weight compounds with IC50 values ranging from 2.821 nM-greater than 1 μM activity. These studies indicate that BCL-xL degrader low molecular weight compounds were capable of inhibiting cell proliferation on various cancer cell lines.

TABLE 11
BCL-xL degrader low molecular weight compounds (IC50s)

| | | COR-L105 | | NCl-H1650 | |
|---|---|---|---|---|---|
| Example | Ligase | IC50 (nM) | Span (Amax) | IC50 (nM) | Span (Amax) |
| 32 | CRBN | — | — | >1000 | — |
| 22 | VHL | 275.3 | 104 | 72.75 | 92.58 |
| 23 | VHL | 182.8 | 100.5 | 35.72 | 71.44 |
| 24 | VHL | 348.2 | 104.3 | 92.93 | 83.85 |
| 25 | CRBN | 174.4 | 87.39 | 38.81 | 74.05 |
| 26 | CRBN | 211.5 | 87.99 | 56.88 | 70.08 |
| 33 | VHL | 93.7 | 76.4 | 8.06 | 89.1 |
| 39 | VHL | 523.7 | 95.3 | 38.43 | 96.37 |
| 52 | VHL | 9.932 | 80.63 | 2.821 | 60.83 |
| 58 | VHL | 481.3 | 85.54 | 187.9 | 98.18 |
| 41 | VHL | 219.5 | 88.22 | 8.826 | 106.1 |
| 54 | VHL | 31.63 | 91.92 | 5.746 | 76.74 |
| 53 | VHL | 33.42 | 97.89 | 4.463 | 79.26 |
| 43 | VHL | 74.82 | 85.91 | 4.222 | 80.26 |
| 37 | VHL | 623.1 | 88.54 | 39.72 | 107.4 |
| 40 | VHL | 107 | 82.13 | 15.58 | 87.28 |
| 56 | VHL | 472.7 | 81.97 | 110.4 | 84.23 |
| 38 | VHL | 83.12 | 87.44 | 8.154 | 87.93 |
| 34 | VHL | 74.25 | 82.72 | 7.67 | 55.36 |
| 55 | VHL | 1165 | 81.01 | 302.3 | 78.81 |
| 57 | VHL | 3629 | 72.49 | 258 | 126.4 |
| 50 | VHL | 286.7 | 90.2 | 25.92 | 93.79 |
| 22 | VHL | 275.3 | 104 | 72.75 | 92.58 |
| 36 | VHL | 295.5 | 94.1 | 37.3 | 68.3 |
| 47 | VHL | 228.3 | 97.59 | 12.53 | 97.1 |
| 46 | VHL | 163.2 | 100.8 | 4.91 | 83.16 |
| 42 | VHL | 290.2 | 91.84 | 3.405 | 136.8 |
| 59 | VHL | 2426 | 105.1 | 118.8 | 77.94 |
| 45 | VHL | 68.35 | 82.99 | 6.271 | 81.22 |
| 51 | VHL | 37.57 | 72.8 | 4.227 | 90.8 |
| 49 | VHL | 311.4 | 92.13 | 44.07 | 89.02 |
| 24 | VHL | 500.3 | 104 | 95.04 | 89.1 |
| 48 | VHL | 111.0 | 94.0 | 23.72 | 88.6 |
| 35 | VHL | 90.19 | 87.9 | 7.70 | 88.51 |
| 23 | VHL | 182.8 | 100.5 | 35.72 | 71.44 |

*n/a: constructs were not tested.
“—”: constructs were tested and inactive.

### E. In Vitro Assessment of Bcl-xL Degrader Compounds Against the Endogenous Cancer Cell Lines: COR-L105 & NCI-H1650 (% Bcl-xL Remaining) Cell Lines

The BCL-xL degrader low molecular weight compounds were tested against two endogenous cancer cell lines:
COR-L105: ECACC No. 92031918 cultured in RPMI-1640+10% FBS
NCI-H1650: (ATCC No. CRL-5883 cultured in RPMI-1640+10% FBS

### Degradation of Endogenous BCL-xL

The ability of the BCL-xL degrader low molecular weight compounds to degrade endogenous BCL-xL was assessed using the ProteinSimple 2-40 kDa Jess Separation Module.

Cell lines were cultured in media that is optimal for their growth at 5% CO₂, 37° C. in a tissue culture incubator. Prior to seeding for the degradation assay, the cells were split at least 2 days before the assay to ensure optimal growth density. On the day of seeding, adherent cells were lifted off tissue culture flasks using 0.25% trypsin. Cell viability and cell density were determined using a cell counter (Vi-Cell XR Cell Viability Analyzer, Beckman Coulter). Cells with higher than 85% viability were seeded for the assay.

The COR-L105 and the NCI-H1650 cell line were seeded in 6-well clear TC-treated multiple well plates (Costar® cat. #3516). Cells were seeded at a density of 2,000,000 cells per well in 2.5 mL of standard growth media. Plates were incubated at 5% CO₂, 37° C. overnight in a tissue culture incubator. On the day of dosing, BCL-xL degrader low molecular weight compounds and a BCL-xL inhibitor control were prepared at 6× in standard growth media. The prepared drug treatments were then added to the cells resulting in a final concentration of 100-300 nM and a final volume of 3.0 mL per well. A control well of untreated cells was also included in the study. Each drug concentration was tested in singlet. Plates were incubated at 5% CO₂, 37° C. for 24 hours in a tissue culture incubator.

After 24 hours, the adherent cells were lifted off tissue culture plates using 0.25% trypsin. Standard growth media was added to quench the trypsin and centrifuged for 5 minutes at 1500 rpm. Cell pellets were washed once with cold PBS and centrifuged for 5 minutes at 1500 rpm. Cells were then washed with 1 mL cold PBS and transferred to a 1.5 mL eppendorf tube. Cells were centrifuged for 10 minutes at 10,000 g at 4° C. in a benchtop mini-centrifuge. The entire supernatant was discarded and cells were resuspended thoroughly in 50 μL of RIPA lysis buffer, generated by the manufacturer's protocol (ProteinSimple, Cat. #CBS401: Lysis Kit-RIPA Buffer for Charge Assays). Lysates were incubated on ice for 30 minutes. Following incubation, lysates were spun in a benchtop mini-centrifuge for 15 minutes at full speed at 4° C. Supernatant was carefully transferred to a new eppendorf tube for downstream analysis.

Protein was quantified using a Pierce™ BCA Protein Assay Kit (ThermoFisher, Cat® 23225) following the manufacturer's instructions. A standard curve was prepared by adding 25 μL of each concentration of reference samples from a Pierce™ Bovine Serum Albumin Standard Pre-Diluted Set (ThermoFisher, cat #23208). Each reference sample was run in duplicate. Testing samples were prepared by diluting cell lysates 1:5 in UltraPure™ DNase/RNase-Free Distilled Water and tested in duplicates of 25 μL each. The BCA working reagent was prepared by addition of 50 parts of BCA Reagent A with 1 part of BCA Reagent B. For each testing well, 200 μL of BCA working reagent was added. The plate was incubated at 37° C. for 30 minutes. A SpectraMax M Series Multi-Mode Microplate Reader was used to measure the absorbance at 562 nm. A standard curve generated by the Pierce™ Bovine Serum Albumin Standard Pre-Diluted Set was used to normalize and calculate the BCA concentration of cell lysates. Only values that fell within the range of the standard curve were accepted for quantification.

The ProteinSimple 2-40 kDa Jess Separation Module was used to detect BCL-xL degradation in treated versus untreated lysates following the manufacturer's instructions (25-Capillary Cartridge, 8 pack, 2-40 kDa, cat #SM W012). Samples were diluted to 1.5 mg/mL in 0.1× Sample Buffer provided by the kit in a total volume of 20 μL. One part 5× Fluorescent Master Mix was combined with four parts diluted lysate for a total volume of 25 μL. Samples were mixed and then denatured at 95° C. for 5 minutes. Samples were stored on ice following denaturation. A Ready-to-use Biotinylated Ladder was prepared following the manufacturer's instructions (EZ Standard Pack, 8 pack, 2-40 kDa, cat #PS-ST05EA). Prepared ladder and samples were loaded into the assay plate following the manufacturer's instructions.

A primary antibody master mix was prepared by diluting anti-BCLxL [54H₆] Rabbit mAb (Cell Signaling Technology, cat #2764) 1:50 and anti-COX IV (4D11-B3-E8) Mouse mAb (Cell Signaling Technology, cat #11967) 1:10 in Antibody Diluent 2 (ProteinSimple, cat #042-203). The prepared primary antibody master mix was loaded into the assay plate following the manufacturer's instructions.

A secondary conjugate master mix was prepared by diluting 20× Anti-Mouse NIR Antibody (ProteinSimple cat #043-821) 1:20 in Anti-Rabbit Secondary (ProteinSimple, cat #042-206). A streptavidin-NIR ladder (ProteinSimple, cat #043-816) was used for detection of COX IV while Chemiluminescence was used to detect BCL-xL. The secondary conjugate master mix and the streptavidin-NIR ladder were loaded into the assay plate following the manufacturer's instructions.

COR-L105 samples were run across three assay plates on three JESS Detection Modules simultaneously. Two samples, COR-L105+300 nM GBR627 (BCL-xL low molecular weight degrader) and COR-L105+300 nM EPS706 (BCL-xL inhibitor control) were included in each assay plate in order to be used as controls across instruments and runs to confirm assay robustness. Reagents were dispensed into the assay plate following the manufacturer's instructions. The plate was centrifuged at 2500 rpm for 5 minutes at room temperature. The desired assay parameters were chosen on the JESS Detection Module to detect NIR and Chemiluminescent signal.

Data was analyzed using Compass for Simple Western Software. For each sample, the amount of BCL-xL was normalized to COX IV for a given sample as determined by the ratio of the area of the Chemiluminescent band at 30 kDa (BCL-xL) to the area of the NIR band at 17 kDa (COX IV). The percent of BCL-xL was calculated by an additional normalization of treated samples to untreated control. The percent of BCL-xL was calculated for the two sample controls, COR-L105+300 nM GBR627 and COR-L105+300 nM EPS706, run across the three assay plates (Table 12). The coefficient of variation (% CV) was calculated for each sample across the three assay plates (% CV=Standard Deviation/Mean)*100). The % CV for both control samples were determined to be within an allowable range to confirm the run results were accurate across instruments and assay plates (<10%). BCL-xL degradation in the representative cancer cell lines COR-L105 and NCI-H1650, when treated with various BCL-xL low molecular weight degrader compounds relative to an untreated cell line controls is summarized in Table 13.

The amount of endogenous BCL-xL remaining after treatment with BCL-xL degrader compounds ranged from 12.06% to greater than 100%. The BCL-xL inhibitor control did not induce BCL-xL degradation with detected BCL-xL levels of 118.33% relative to untreated control. Less than 50% of BCL-xL was detected when COR-L105 was dosed with 12 out of the 22 compounds tested. Example 51 was the most potent degrader, with 12.06% BCL-xL remaining relative to untreated control after 24 hours at a 300 nM dose. Three BCL-xL degrader compounds did not have a degradation effect, with >90% BCL-xL remaining versus untreated control: Example 36, Example 49 and Example 35.

These studies indicate that BCL-xL degrader low molecular weight compounds were capable of degrading endogenous BCL-xL in a representative cancer cell line.

TABLE 12
Coefficient of variation of control samples across assay plates and detection modules
% BCLxL at 24 Hrs. + 300 nM LMW vs. Untreated Lysate

| | | | | | Standard | |
|---|---|---|---|---|---|---|
| Sample | Plate 1 | Plate 2 | Plate 3 | Average | Deviation | % CV |
| COR-L105 + 300 nM EPS706 | 113.89 | 116.17 | 124.91 | 118.33 | 5.82 | 4.92 |
| COR-L105 + 300 nM GBR627 | 23.48 | 21.14 | 23.25 | 22.62 | 1.29 | 5.70 |

TABLE 13
Measured BCL-XL in COR-L105 treated with BCL-xL
LMW degraders versus untreated cell controls.

| | | COR-L105 | | NCl-H1650 | |
|---|---|---|---|---|---|
| | | Dose | % | Dose | % |
| | | at 24 | BCL- | at 24 | BCL- |
| | | Hours | xL vs. | Hours | xL vs. |
| Example | Ligase | (nM) | Untreated | (nM) | Untreated |
| 33 | VHL | 300 | 64.83 | n/a | n/a |
| 39 | VHL | 300 | 63.62 | n/a | n/a |
| 52 | VHL | 300 | 28.63 | n/a | n/a |
| 41 | VHL | 300 | 30.31 | n/a | n/a |
| 54 | VHL | 300 | 47.89 | n/a | n/a |
| 53 | VHL | 300 | 48.29 | n/a | n/a |
| 43 | VHL | 300 | 19.88 | n/a | n/a |
| 37 | VHL | 300 | 82.4 | n/a | n/a |
| 40 | VHL | 300 | 71.41 | n/a | n/a |
| 44 | VHL | 300 | 58.31 | n/a | n/a |
| 38 | VHL | 300 | 22.11 | n/a | n/a |
| 34 | VHL | 300 | 74.51 | n/a | n/a |
| 50 | VHL | 300 | 87.97 | n/a | n/a |
| 36 | VHL | 300 | 105.51 | n/a | n/a |
| 47 | VHL | 300 | 22.26 | n/a | n/a |
| 46 | VHL | 300 | 22.62 | 10 | 16.23 |
| 42 | VHL | 300 | 33.28 | n/a | n/a |
| 45 | VHL | 300 | 40.06 | n/a | n/a |
| 51 | VHL | 300 | 12.06 | 10 | 9.22 |
| 49 | VHL | 300 | 93.55 | n/a | n/a |
| 24 | VHL | 300 | 39.98 | n/a | n/a |
| 48 | VHL | n/a | n/a | 10 | 1.05 |
| 35 | VHL | 300 | 108.82 | n/a | n/a |

*n/a: constructs were not tested.

### F. Effect of Bcl-xL Degrader Compounds on MOLT-4 Cell Viability Using MTT or CTG Assays

The MTT colorimetric assay is based on the mitochondrial reduction of tetrazolium salt by living cells. The viable cell number is proportional to the production of formazan salts, which can be read spectrophotometrically at 540 nm. The CTG luminescent assay (CellTiter GLO, Promega) is based on a reagent which lyses cells and measures total adenosine triphosphate (ATP) content. The viable cell number is proportional to the production of ATP.

MOLT-4 cells were purchased from ATCC and cultivated in RPMI 1640 supplemented with 10% heat inactivated fetal bovine serum, penicillin (100 IU/mi), streptomycin (100 μg/ml) and L-glutamine (2 mM). Cells were cultured at 37° C. in a humidified atmosphere containing 5% CO₂.

For MTT assay, cells were seeded in 96 microwell plates (150 μL per well) and exposed to the compounds for 48 h (3.16 fold serially diluted; 9 concentrations each, triplicates). At the end of incubation time, 15 μL of MTT solution (5 mg/ml) were added per well and the cells were incubated for another 4 h. Then, 100 μL of 10% Sodium Dodecyl Sulfate (SDS)/HCl 10 mM were added per well and the plate was incubated overnight, before measurement of optical density at 540 nm.

For CTG assay, cells were seeded in 384 microwell plates (40 μL per well) and exposed to the compounds for 48 h (11-point serial dilution with 5-fold factor, triplicates). At the end of incubation time, 40 μL of CTG solution were added per well. Wells were mixed thoroughly, and plates were incubated at room temperature for 10 minutes to stabilize luminescent signals prior to reading using a luminescence reader (PHERAStar FSX Reader, BMG Labtech).

IC₅₀ₛ were calculated using standard four-parametric curve fitting. IC₅₀ is defined as the compound concentration at which the MTT or CTG signal is reduced to 50% of that measured for the control. Results shown in Table 14 represent the mean of at least 2 independent experiments.

As shown in Table 14, all the tested compounds are active and induced a decrease in the viability of MOLT-4 cells in MTT and/or CTG assay.

TABLE 14
Effect of Bcl-XL Degrader Compounds on
MOLT-4 Cell Viability Using MTT Assay

| | MOLT-4 MTT/48 h | MOLT-4 CTG/48 h |
|---|---|---|
| Example | (IC₅₀) [M] | (IC₅₀) [M] |
| 1 | 6.36E−10 | 3.41E−09 |
| 2 | 1.06E−09 | n/a |
| 3 | 3.21E−08 | n/a |
| 4 | 8.5E−10 | n/a |
| 5 | 3.58E−09 | 1.58E−08 |
| 6 | 6.57E−09 | 2.87E−08 |
| 7 | 4.93E−10 | n/a |
| 8 | 6.56E−09 | 3.43E−08 |
| 9 | 5.21E−09 | 1.29E−08 |
| 10 | 3.98E−10 | n/a |
| 11 | 4.29E−09 | 2.03E−08 |
| 12 | 9.49E−09 | 1.99E−08 |
| 13 | 1.35E−08 | 3.39E−08 |
| 14 | 1.15E−09 | 3.51E−09 |
| 15 | 5.67E−09 | 2.26E−08 |
| 16 | 1.07E−09 | n/a |
| 17 | 3.84E−09 | n/a |
| 18 | 2.72E−09 | n/a |
| 19 | 1.32E−08 | n/a |
| 20 | 4.49E−09 | n/a |
| 21 | 1.15E−08 | 2.36E−08 |
| 22 | 4.7E−09 | n/a |
| 23 | 1.05E−08 | n/a |
| 24 | 2.64E−09 | n/a |
| 25 | 1.09E−09 | n/a |
| 26 | 2.09E−08 | 2.60E−08 |
| 27 | 6.69E−07 | n/a |
| 28 | 8.63E−09 | n/a |
| 29 | 8.75E−07 | 1.63E−06 |
| 30 | 1.33E−07 | 4.77E−07 |
| 31 | 8.04E−09 | 7.00E−09 |
| 32 | 6.42E−08 | n/a |
| 60 | 7.17E−10 | n/a |
| 61 | 1.64E−08 | n/a |
| 62 | 2.41E−08 | n/a |
| 63 | 8.64E−08 | n/a |
| 64 | 9.28E−08 | 1.18E−07 |
| 65 | 1.56E−07 | 1.40E−07 |
| 66 | 2.71E−07 | 9.29E−07 |
| 67 | n/a | 2.42E−08 |
| 68 | n/a | 1.53E−08 |
| 69 | n/a | 5.14E−09 |
| 70 | n/a | 7.48E−09 |
| 71 | n/a | 1.02E−08 |
| 72 | n/a | 1.67E−08 |
| 73 | n/a | 2.40E−08 |
| 74 | n/a | n/a |
| 75 | n/a | 5.85E−09 |
| 76 | n/a | 8.34E−09 |
| 77 | n/a | 4.54E−09 |
| 78 | n/a | 4.42E−09 |
| 79 | n/a | 6.06E−09 |
| 80 | n/a | 6.11E−09 |
| 81 | n/a | 1.51E−08 |
| 82 | n/a | 1.00E−08 |
| 83 | n/a | 4.72E−09 |
| 84 | n/a | 1.81E−08 |
| 85 | n/a | 4.17E−09 |
| 86 | n/a | 1.37E−08 |
| 87 | n/a | 3.47E−08 |
| 88 | n/a | 1.63E−08 |
| 89 | n/a | 5.76E−08 |
| 90 | n/a | 1.68E−08 |
| 91 | n/a | 1.27E−08 |
| 92 | n/a | 1.49E−08 |
| 93 | n/a | 1.96E−08 |
| 94 | n/a | 1.44E−08 |
| 95 | n/a | 5.77E−09 |
| 96 | n/a | 4.36E−09 |
| 97 | n/a | 3.76E−09 |
| 98 | n/a | 4.74E−09 |
| 99 | n/a | 4.54E−09 |
| 100 | n/a | 8.41E−09 |
| 101 | n/a | 7.68E−09 |
| 102 | n/a | 4.07E−09 |
| 103 | n/a | 6.34E−08 |
| 104 | n/a | 1.74E−09 |
| 105 | n/a | 1.75E−09 |
| 106 | n/a | 2.63E−09 |
| 107 | n/a | 4.72E−09 |
| 108 | n/a | 9.70E−09 |
| 109 | n/a | 7.84E−09 |
| 110 | n/a | 2.39E−08 |
| 111 | n/a | 2.62E−09 |
| 112 | n/a | 1.57E−08 |
| 113 | n/a | 2.06E−08 |
| 114 | n/a | 2.48E−08 |
| 115 | n/a | 3.93E−09 |
| 116 | n/a | 1.61E−08 |
| 117 | n/a | 8.25E−09 |
| 118 | n/a | 7.83E−09 |
| 119 | n/a | 4.78E−09 |
| 120 | n/a | 7.29E−09 |
| 121 | n/a | 9.99E−09 |
| 122 | n/a | 4.60E−09 |
| 123 | n/a | 5.60E−09 |
| 124 | n/a | 3.69E−09 |
| 125 | n/a | 9.34E−09 |
| 126 | n/a | 2.40E−07 |
| 127 | n/a | n/a |
| 128 | n/a | 6.70E−08 |
| 129 | n/a | 5.30E−09 |
| 130 | n/a | 4.04E−09 |
| 131 | n/a | 1.39E−08 |
| 132 | n/a | 2.66E−08 |
| 133 | n/a | 1.02E−08 |
| 134 | n/a | 7.19E−09 |
| 135 | 3.51E−09 | n/a |
| 136 | 1.45E−09 | n/a |
| 137 | 1.62E−08 | n/a |
| 138 | 3.31E−09 | n/a |
| 139 | 3.52E−09 | n/a |
| 140 | 3.11E−10 | n/a |
| 141 | n/a | 1.04E−08 |
| 142 | n/a | 3.76E−09 |
| 143 | n/a | 5.28E−09 |
| 144 | n/a | n/a |
| 145 | n/a | 2.41E−09 |
| 146 | n/a | 3.00E−09 |
| 147 | n/a | 5.34E−09 |
| 148 | n/a | 4.51E−09 |
| 149 | 3.58E−07 | n/a |
| 150 | 1.93E−08 | n/a |
| 151 | 3.17E−07 | n/a |
| 152 | 8.21E−08 | n/a |
| 153 | 2.75E−08 | n/a |
| 154 | n/a | 6.59E−09 |
| 155 | n/a | 2.21E−08 |
| 156 | n/a | 6.48E−09 |
| 157 | n/a | 7.62E−09 |
| 173 | n/a | 3.56E−08 |
| 174 | n/a | 2.60E−08 |

### G. Effect of Bcl-xL PROTACs on HCT116 Bcl-xL Degradation Using Capillary-Based Immunoassay

HCT116 cells were purchased from ATCC and cultivated in RPMI 1640 supplemented with 10% heat inactivated fetal bovine serum, penicillin (100 IU/ml), streptomycin (100 μg/ml) and L-glutamine (2 mM). Cells were cultured at 37° C. in a humidified atmosphere containing 5% CO₂. Cells were seeded in 96 microwell plates, 24 h later cells were treated with compounds (2-point 10/100 nM and/or 9-point serial dilution to determine DC₅₀ quadruplicates) during 24 h and harvested in lysis buffer containing 10 mM Hepes (pH 7.5), 150 mM KCl, 5 mM MgCl2, 1 mM EDTA, 0.4% TritonX100, protease and phosphatase inhibitors cocktails (Calbiochem).

Protein concentration in the supernatants was determined using the Pierce BCA protein assay kit (Thermo Fisher Scientific). Lysates (1.5 μg/μL) were analyzed using an automated capillary electrophoresis WES system with 2-40 kDa Wes Separation Module (25 capillary cartridges, Proteinsimple), in accordance with the manufacturer's protocols. Antibodies against the following proteins were used: Bcl-xL (#2764, Cell Signaling Technology, 1:50) and cofilin (#5175, Cell Signaling Technology, 1:1000). Signals were detected with an HRP-conjugated secondary anti-rabbit antibody (#042-206, Proteinsimple) and were quantified using Compass software (version 4.0, Proteinsimple).

Bcl-xL level was calculated by normalizing Bcl-xL values to cofilin and expressed as a percentage of the normalized value of the untreated cells. DC₅₀ were calculated using standard four-parametric curve fitting. DC₅₀ is defined as the compound concentration at which Bcl-xL is reduced to 50% of that measured for the control. Measured levels of Bcl-xL protein of HCT116 cells treated with the tested compounds at 2-point 10 nM/100 nM and/or 9-point for DC₅₀ determination are shown in Table 15.

As shown in Table 15, all the tested compounds induced a decrease in the level of Bcl-xL protein of HCT116 cells in WES assay.

TABLE 15
Effect of Bcl-XL PROTACs on HCT116 Bcl-xL degradation using
capillary-based immunoassay

| | Bcl-xL % @10 nM | Bcl-xL % @100 nM | Bcl-xL DC50 (M) |
|---|---|---|---|
| Example | WES HCT116 24 h | WES HCT116 24 h | WES HCT116 24 h |
| 1 | 21 | 9 | 3.29E−10 |
| 2 | 26 | 11 | 2.86E−10 |
| 3 | 23 | 17 | 4.21E−09 |
| 4 | 33 | 14 | 2.79E−10 |
| 5 | 54 | 19 | 2.45E−09 |
| 6 | 69 | 22 | 2.94E−09 |
| 7 | 52 | 34 | 7.42E−10 |
| 8 | 54 | 37 | n/a |
| 9 | 61 | 41 | 1.05E−08 |
| 10 | 48 | 43 | >3.16E−07 |
| 11 | 83 | 60 | n/a |
| 12 | 69 | 69 | n/a |
| 13 | 79 | 57 | n/a |
| 14 | 81 | 86 | n/a |
| 15 | 39 | 23 | 4.62E−10 |
| 16 | 7 | 7 | 4.7E−10 |
| 17 | 32 | 9 | 4.88E−10 |
| 18 | 52 | 52 | 6.6E−09 |
| 19 | 104 | 88 | n/a |
| 20 | 51 | 31 | n/a |
| 21 | 73 | 35 | n/a |
| 22 | 68 | 54 | >3.16E−07 |
| 23 | 82 | 67 | n/a |
| 24 | 75 | 44 | >3.16E−07 |
| 25 | 59 | 55 | >3.16E−07 |
| 26 | 63 | 51 | >3.16E−07 |
| 27 | 60 | 49 | n/a |
| 28 | 81 | 46 | n/a |
| 29 | 64 | 67 | n/a |
| 30 | 118 | 73 | n/a |
| 31 | 11 | 6 | 1.22E−09 |
| 32 | 83 | 53 | n/a |
| 60 | 19 | 23 | 1.1E−10 |
| 61 | 46 | 16 | 7.49E−09 |
| 62 | 47 | 7 | 1.36E−08 |
| 63 | 61 | 39 | 5.3E−08 |
| 64 | 59 | 40 | 4.62E−08 |
| 65 | 90 | 78 | n/a |
| 66 | 91 | 62 | >3.16E−07 |
| 67 | n/a | n/a | >3.16E−07 |
| 68 | n/a | n/a | >3.16E−07 |
| 69 | n/a | n/a | 7.11E−10 |
| 70 | 101 | 90 | n/a |
| 71 | n/a | n/a | >3.16E−07 |
| 72 | n/a | n/a | 1.33E−08 |
| 73 | n/a | n/a | 7.69E−09 |
| 74 | n/a | n/a | 1.33E−10 |
| 75 | n/a | n/a | n/a |
| 76 | n/a | n/a | 2.8E−10 |
| 77 | n/a | n/a | >3.16E−07 |
| 78 | n/a | n/a | 1.21E−10 |
| 79 | n/a | n/a | 3.99E−10 |
| 80 | n/a | n/a | 3.57E−09 |
| 81 | n/a | n/a | >3.16E−07 |
| 82 | n/a | n/a | >3.16E−07 |
| 83 | n/a | n/a | 4.49E−09 |
| 84 | n/a | n/a | >3.16E−07 |
| 85 | n/a | n/a | 2.91E−10 |
| 86 | n/a | n/a | n/a |
| 87 | n/a | n/a | >3.16E−07 |
| 88 | n/a | n/a | >3.16E−07 |
| 89 | n/a | n/a | >3.16E−07 |
| 90 | n/a | n/a | 1.33E−09 |
| 91 | n/a | n/a | 4.07E−10 |
| 92 | n/a | n/a | 9.54E−10 |
| 93 | n/a | n/a | 9.51E−10 |
| 94 | n/a | n/a | 1.06E−09 |
| 95 | n/a | n/a | 2.85E−09 |
| 96 | n/a | n/a | >3.16E−07 |
| 97 | n/a | n/a | 3.56E−10 |
| 98 | n/a | n/a | 3.62E−10 |
| 99 | n/a | n/a | n/a |
| 100 | n/a | n/a | n/a |
| 101 | n/a | n/a | 6.12E−10 |
| 102 | n/a | n/a | 4.48E−10 |
| 103 | n/a | n/a | 1.36E−08 |
| 104 | n/a | n/a | 1.84E−11 |
| 105 | n/a | n/a | 4.47E−11 |
| 106 | n/a | n/a | 2.11E−10 |
| 107 | n/a | n/a | 7.39E−10 |
| 108 | n/a | n/a | >3.16E−07 |
| 109 | n/a | n/a | 1.55E−10 |
| 110 | n/a | n/a | 1.14E−08 |
| 111 | n/a | n/a | 4E−10 |
| 112 | n/a | n/a | 4.95E−10 |
| 113 | n/a | n/a | >3.16E−07 |
| 114 | n/a | n/a | 2.67E−09 |
| 115 | n/a | n/a | 2.66E−10 |
| 116 | n/a | n/a | 3.6E−09 |
| 117 | n/a | n/a | 1.17E−09 |
| 118 | n/a | n/a | >3.16E−07 |
| 119 | n/a | n/a | 6.81E−10 |
| 120 | n/a | n/a | 1.68E−09 |
| 121 | n/a | n/a | 2.57E−09 |
| 122 | n/a | n/a | >3.16E−07 |
| 123 | n/a | n/a | >3.16E−07 |
| 124 | n/a | n/a | 1.01E−09 |
| 125 | n/a | n/a | 4.87E−10 |
| 126 | n/a | n/a | >3.16E−07 |
| 127 | n/a | n/a | n/a |
| 128 | n/a | n/a | n/a |
| 129 | n/a | n/a | 7.34E−09 |
| 130 | n/a | n/a | 8.57E−10 |
| 131 | n/a | n/a | 1.34E−09 |
| 132 | 108 | 103 | n/a |
| 133 | 112 | 105 | n/a |
| 134 | n/a | n/a | 5.64E−10 |
| 135 | n/a | n/a | 9.78E−10 |
| 136 | n/a | n/a | 2.71E−09 |
| 137 | n/a | n/a | >3.16E−07 |
| 138 | n/a | n/a | 2.12E−08 |
| 139 | n/a | n/a | 3.97E−10 |
| 140 | n/a | n/a | 3.08E−09 |
| 141 | n/a | n/a | >3.16E−07 |
| 142 | n/a | n/a | 3.41E−10 |
| 143 | n/a | n/a | >3.16E−07 |
| 144 | n/a | n/a | n/a |
| 145 | n/a | n/a | 2.17E−09 |
| 146 | n/a | n/a | 9.86E−10 |
| 147 | n/a | n/a | >3.16E−07 |
| 148 | n/a | n/a | 1.37E−09 |
| 149 | n/a | n/a | n/a |
| 150 | n/a | n/a | 1.45E−08 |
| 151 | 78 | 91 | n/a |
| 152 | n/a | n/a | 1.14E−08 |
| 153 | 59 | 40 | n/a |
| 154 | n/a | n/a | 1.03E−08 |
| 155 | n/a | n/a | n/a |
| 156 | n/a | n/a | 1.45E−08 |
| 157 | n/a | n/a | 1.88E−08 |
| 173 | n/a | n/a | >3.16E−07 |
| 174 | n/a | n/a | >3.16E−07 |

### H. Evaluation of BCLxL Degrader Compounds on EBC-1 Cell Proliferation

The ability of the BCL-xL degrader low molecular weight compounds to inhibit cell proliferation and survival was assessed using the Promega CellTiter-Glo® proliferation assay on the EBC-1 lung cancer cell line (RCB1965, obtained from RIKEN.) The cells were cultured in media that is optimal for their growth at 5% CO₂, 37° C. in a tissue culture incubator. Prior to seeding for the proliferation assay, the cells were split at least 2 days before the assay to ensure optimal growth density. On the day of seeding, cells were lifted off tissue culture flasks using 0.25% trypsin. Cell viability and cell density were determined using a cell counter (Vi-Cell XR Cell Viability Analyzer, Beckman Coulter). Cells with higher than 85% viability were seeded in white clear bottom 384-well plates (Greiner cat #781098) at a density of 1000 cells per well in 50 μL of standard growth media. Plates were incubated at 37° C. overnight in a tissue culture incubator.

The BCL-xL degrader low molecular weight compounds were prepared to desired concentrations. A series of 10 dilutions were made for each prepared compound. The prepared compounds were then added to the cells resulting in final concentrations of 0.508 nM-10 μM. An acoustic transfer device (Echo555, Beckman Coulter) was used to add the compounds to the cells. Each treatment was tested in triplicate assay plates. Plates were incubated at 37° C. overnight or for 5 days in a tissue culture incubator. The ability of the compounds to inhibit cell proliferation and survival was assessed using the Promega CellTiter-Glo® proliferation assay. Plates were incubated at room temperature for 20 minutes to stabilize luminescent signals prior to reading using a multimode plate reader (PHERAstar FSX Microplate Reader, BMG Labtech).

To evaluate the effect of the compound treatments, luminescent counts of untreated cells were taken the day after seeding (Day 0 readings), and after 5 days of treatment (Day 5 readings). The Day 5 readings of the untreated cells were compared to the Day 0 readings. Assays with at least one cell doubling during the incubation period were considered valid. To evaluate the effect of the drug treatments, luminescent counts from wells containing untreated cells (100% viability) were used to normalize treated samples. The concentrations of treatment required to inhibit 50% of cell growth or survival (G150) were calculated using a four parameter logistic regression equation.

### I. Evaluation of BCLxL Degrader Compounds on NCI-H1650 BCLxL Protein Levels

The ability of the BCL-xL degrader low molecular weight compounds to degrade BCL-xL protein was assessed using the Promega Nano-Glo® HiBiT Lytic Detection System. The NCI-H1650 (ATCC No. CRL-5883) was engineered to express HiBiT-tagged BCL-xL. The NCI-H1650 HiBiT cell line was cultured in media that is optimal for their growth at 5% CO₂, 37° C. in a tissue culture incubator. Prior to seeding for the protein level assay, the cells were split at least 2 days before the assay to ensure optimal growth density. On the day of seeding, cells were lifted off tissue culture flasks using 0.25% trypsin. Cell viability and cell density were determined using a cell counter (Vi-Cell XR Cell Viability Analyzer, Beckman Coulter). Cells with higher than 85% viability were seeded in white clear bottom 384-well plates (Greiner cat #781098) at a density of 5000 cells per well in 30 μL of standard growth media. Plates were incubated at 37° C. overnight in a tissue culture incubator.

The BCL-xL degrader low molecular weight compounds were prepared to desired concentrations. A series of 10 dilutions were made for each prepared compound. The prepared compounds were then added to the cells resulting in final concentrations of 0.508 nM-10 μM. An acoustic transfer device (Echo555, Beckman Coulter) was used to add the compounds to the cells. Each treatment was tested in triplicate assay plates. Plates were incubated at 37° C. overnight in a tissue culture incubator.

BCL-xL degradation was assessed through the addition of 30 μL of Nano-Glo® HiBiT Lytic Reagent (Promega, cat #N₃₀₄₀), a reagent which lyses cells and contains a furimazine substrate and the large subunit (LgBiT) used in NanoLuc® Binary Technology. The LgBiT subunit having high affinity to a HiBiT-tagged protein will bind HiBiT, if present, resulting in complex formation of a luminescent NanoBiT® enzyme. This reaction will emit a luminescent signal proportional to the amount of HiBiT-tagged BCL-xL present in the cell lysate. Plates were incubated at room temperature for 20 minutes to stabilize luminescent signals prior to reading using a luminescence reader (PHERAstar FSX Microplate Reader, BMG Labtech).

To evaluate the effect of the drug treatments, luminescent counts from wells containing untreated cells (100% HiBiT-tagged BCL-xL) were used to normalize treated samples. The concentrations of treatment required to inhibit 50% were calculated using a four parameter logistic regression equation.

TABLE 16
Effect of Bcl-XL degrader compounds on Ebc-1 cell viability using
CTG assay and Bcl-xL protein degradation using Hibit assay.

| | | | Bcl-xl |
|---|---|---|---|
| | | CTG 5 day | HiBIT |
| Example | | Ebc1 | NCIH1650 |
| number | Ligase | GI₅₀ (μM) | DC₅₀ (μM) |
| 1 | VHL | 0.005 | 0.001 |
| 2 | VHL | 0.013 | 0.001 |
| 3 | VHL | 0.009 | 0.003 |
| 4 | VHL | 0.001 | 0.0004 |
| 5 | VHL | 0.023 | 0.002 |
| 6 | VHL | 0.013 | 0.003 |
| 7 | VHL | 0.002 | 0.001 |
| 8 | VHL | n/a | n/a |
| 9 | CRBN | n/a | n/a |
| 10 | CRBN | n/a | n/a |
| 11 | CRBN | n/a | n/a |
| 12 | CRBN | n/a | n/a |
| 13 | CRBN | n/a | n/a |
| 14 | CRBN | n/a | n/a |
| 15 | VHL | 0.021 | >1.670 |
| 16 | VHL | 0.009 | 0.003 |
| 17 | VHL | 0.031 | >1.670 |
| 18 | VHL | n/a | n/a |
| 19 | VHL | 0.025 | >1.670 |
| 20 | VHL | n/a | n/a |
| 21 | VHL | n/a | n/a |
| 22 | VHL | n/a | n/a |
| 23 | VHL | n/a | n/a |
| 24 | VHL | n/a | n/a |
| 25 | VHL | n/a | n/a |
| 26 | CRBN | n/a | n/a |
| 27 | CRBN | n/a | n/a |
| 28 | CRBN | n/a | n/a |
| 29 | VHL | n/a | n/a |
| 30 | CRBN | n/a | n/a |
| 31 | VHL | 0.001 | 0.0004 |
| 32 | CRBN | n/a | n/a |
| 33 | VHL | 0.033 | >1.670 |
| 34 | VHL | n/a | n/a |
| 35 | VHL | 0.019 | >1.670 |
| 36 | VHL | n/a | n/a |
| 37 | VHL | n/a | n/a |
| 38 | VHL | n/a | n/a |
| 39 | VHL | n/a | n/a |
| 40 | VHL | n/a | n/a |
| 41 | VHL | n/a | n/a |
| 42 | VHL | 0.021 | >1.670 |
| 43 | VHL | n/a | n/a |
| 44 | VHL | n/a | n/a |
| 45 | VHL | n/a | n/a |
| 46 | VHL | 0.007 | 0.002 |
| 47 | VHL | 0.076 | 0.009 |
| 48 | VHL | 0.012 | 0.001 |
| 49 | VHL | n/a | n/a |
| 50 | VHL | n/a | n/a |
| 51 | VHL | 0.001 | 0.0003 |
| 52 | VHL | n/a | n/a |
| 53 | VHL | n/a | n/a |
| 54 | VHL | n/a | n/a |
| 55 | VHL | n/a | n/a |
| 56 | VHL | n/a | n/a |
| 57 | VHL | 0.005 | 0.018 |
| 58 | VHL | n/a | n/a |
| 59 | VHL | n/a | n/a |
| 60 | CRBN | 0.001 | >1.670 |
| 61 | CRBN | n/a | n/a |
| 62 | CRBN | n/a | n/a |
| 63 | CRBN | n/a | n/a |
| 64 | CRBN | n/a | n/a |
| 65 | CRBN | n/a | n/a |
| 66 | VHL | 0.520 | 0.200 |
| 67 | CRBN | n/a | n/a |
| 68 | CRBN | n/a | n/a |
| 69 | CRBN | n/a | n/a |
| 70 | CRBN | n/a | n/a |
| 71 | CRBN | n/a | n/a |
| 72 | VHL | 0.179 | >1.670 |
| 73 | VHL | 0.186 | 0.013 |
| 74 | CRBN | n/a | n/a |
| 75 | CRBN | n/a | n/a |
| 76 | VHL | 0.134 | 0.003 |
| 77 | VHL | 0.167 | >1.670 |
| 78 | CRBN | n/a | n/a |
| 79 | VHL | 0.066 | >1.670 |
| 80 | VHL | 0.092 | >1.670 |
| 81 | VHL | 0.203 | >1.670 |
| 82 | VHL | 0.157 | >1.670 |
| 83 | VHL | n/a | n/a |
| 84 | VHL | 0.171 | >1.670 |
| 85 | VHL | 0.064 | 0.005 |
| 86 | VHL | 0.018 | >1.670 |
| 87 | VHL | 0.216 | >1.670 |
| 88 | VHL | 0.096 | >1.670 |
| 89 | VHL | 0.435 | 0.013 |
| 90 | CRBN | n/a | n/a |
| 91 | CRBN | n/a | n/a |
| 92 | CRBN | n/a | n/a |
| 93 | CRBN | n/a | n/a |
| 94 | VHL | 0.112 | >1.670 |
| 95 | CRBN | 2.665 | >1.670 |
| 96 | IAP | n/a | n/a |
| 97 | IAP | n/a | n/a |
| 98 | IAP | n/a | n/a |
| 99 | IAP | n/a | n/a |
| 100 | IAP | n/a | n/a |
| 101 | IAP | n/a | n/a |
| 102 | IAP | n/a | n/a |
| 103 | VHL | 0.378 | >1.670 |
| 104 | CRBN | n/a | n/a |
| 105 | CRBN | n/a | n/a |
| 106 | CRBN | n/a | n/a |
| 107 | CRBN | n/a | n/a |
| 108 | VHL | 0.081 | >1.670 |
| 109 | VHL | 0.098 | 0.001 |
| 110 | VHL | 0.379 | 0.002 |
| 111 | VHL | 0.041 | 0.0005 |
| 112 | VHL | 0.437 | 0.0005 |
| 113 | VHL | 0.049 | >1.670 |
| 114 | VHL | 1.716 | 0.001 |
| 115 | CRBN | n/a | n/a |
| 116 | VHL | 0.045 | 0.001 |
| 117 | VHL | 0.089 | >1.670 |
| 118 | VHL | 0.084 | >1.670 |
| 119 | CRBN | 0.069 | >1.670 |
| 120 | VHL | 0.119 | 0.001 |
| 121 | VHL | 0.190 | 0.001 |
| 122 | VHL | 0.046 | >1.670 |
| 123 | VHL | 0.037 | >1.670 |
| 124 | VHL | 0.025 | 0.001 |
| 125 | VHL | 0.081 | 0.001 |
| 126 | VHL | 1.433 | >1.670 |
| 127 | VHL | 0.462 | 0.033 |
| 128 | VHL | 0.869 | 0.018 |
| 129 | VHL | 0.003 | 0.001 |
| 130 | VHL | 0.002 | 0.001 |
| 131 | VHL | 0.008 | 0.001 |
| 132 | CRBN | n/a | n/a |
| 133 | VHL | 0.004 | >1.670 |
| 134 | VHL | 0.002 | 0.001 |
| 135 | VHL | 0.004 | 0.001 |
| 136 | VHL | 0.001 | 0.001 |
| 137 | VHL | 0.004 | >1.670 |
| 138 | VHL | n/a | >1.670 |
| 139 | VHL | 0.007 | 0.003 |
| 140 | CRBN | n/a | n/a |
| 141 | CRBN | n/a | n/a |
| 142 | CRBN | n/a | n/a |
| 143 | CRBN | n/a | n/a |
| 144 | CRBN | n/a | n/a |
| 145 | VHL | 0.001 | 0.002 |
| 146 | VHL | 0.003 | >1.670 |
| 147 | CRBN | n/a | n/a |
| 148 | VHL | 0.007 | 0.001 |
| 149 | CRBN | n/a | n/a |
| 150 | CRBN | n/a | n/a |
| 151 | VHL | n/a | n/a |
| 152 | VHL | 0.121 | >1.670 |
| 153 | CRBN | n/a | n/a |
| 154 | VHL | 0.222 | >1.670 |
| 155 | VHL | 0.431 | >1.670 |
| 156 | VHL | 0.027 | 0.228 |
| 157 | VHL | n/a | n/a |
| 158 | VHL | 0.010 | >1.670 |
| 159 | VHL | 0.008 | >1.670 |
| 160 | VHL | 0.058 | 0.002 |
| 161 | VHL | 0.008 | 0.0004 |
| 162 | VHL | 0.222 | >1.670 |
| 163 | VHL | 0.022 | 0.006 |
| 164 | VHL | 0.035 | 0.002 |
| 165 | VHL | 0.035 | >1.670 |
| 166 | VHL | 0.068 | 0.001 |
| 167 | VHL | 0.136 | 0.002 |
| 168 | VHL | 0.073 | >1.670 |
| 169 | VHL | 0.006 | >1.670 |
| 170 | VHL | 0.013 | 0.001 |
| 171 | VHL | 0.121 | >1.670 |

1. A compound of Formula (A):
D-L-DSM (A),
or an enantiomer, a diastereoisomer, and/or a pharmaceutically acceptable salt of any one of the foregoing, wherein:
DSM is a degradation signaling compound covalently attached to the linker L;
L is a linker that covalently attaches DSM to D; and
D is a Bcl-xL inhibitor compound of Formula (I) or Formula (II) covalently attached to the
linker L: 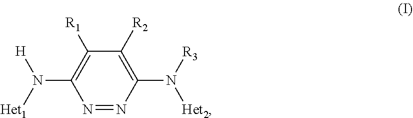
or an enantiomer, a diastereoisomer, and/or a pharmaceutically acceptable salt of any one of the foregoing, wherein:
R₁ and R₂ independently of one another represent a group selected from the group consisting of: hydrogen; a linear or branched C₁-C₆alkyl optionally substituted by a hydroxyl or a C₁-C₆alkoxy group; a C₃-C₆cycloalkyl; a trifluoromethyl; and a linear or branched C₁-C₆alkylene-heterocycloalkyl wherein the heterocycloalkyl group is optionally substituted by a linear or branched C₁-C₆alkyl group;
or R₁ and R₂ form with the carbon atoms carrying them a C₃-C₆cycloalkylene group,
R₃ represents a group selected from the group consisting of: hydrogen; a C₃-C₆cycloalkyl; a linear or branched C₁-C₆alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:
—X₁-≡CH,
Rₐ and R_{b} independently of one another represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl groups; a C₁-C₆alkylene-SO₂OH; a C₁-C₆alkylene-SO₂O⁻; a C₁-C₆alkylene-COOH; a C₁-C₆alkylene-PO(OH)₂; a C₁-C₆alkylene-NR_{d}Rₑ; a C₁-C₆alkylene-N⁺R_{d}RₑR_{f}; a C₁-C₆alkylene-phenyl wherein the phenyl may be substituted by a C₁-C₆alkoxy group; and the group: 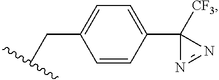 [SMILES: *Cc1ccc(C2(C(F)(F)F)N=N2)cc1]
or Rₐ and R_{b} form with the nitrogen atom carrying them a cycle B₁;
or Rₐ, R_{b} and R_{c} form with the nitrogen atom carrying them a bridged C₃-C₈hetero cycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a cycle B₂,
or R_{d}, Rₑ and R_{f} form with the nitrogen atom carrying them a bridged C₃-C₈hetero cycloalkyl,
Het₁ represents a group selected from the group consisting of: 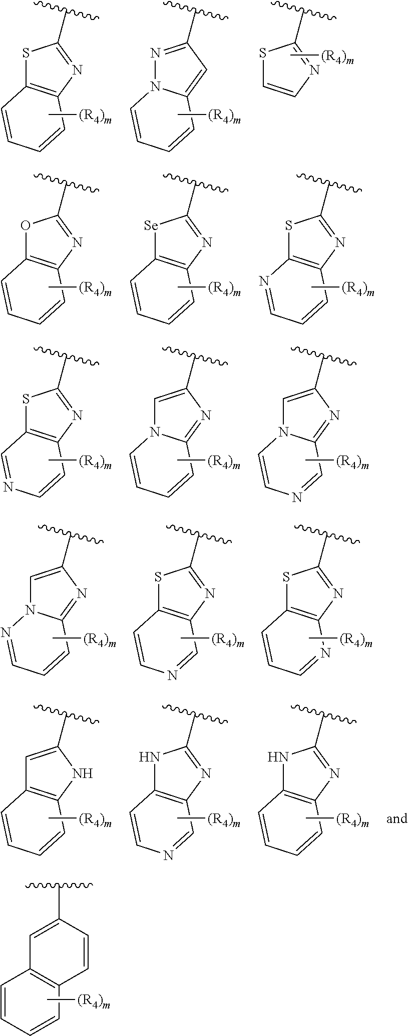
Het₂ represents a group selected from the group consisting of: 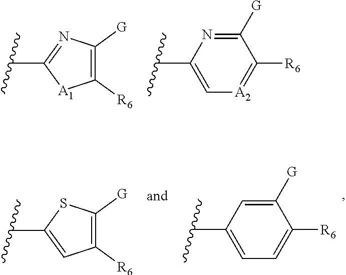
A₁ is —NH—, —N(C₁-C₃alkyl), O, S or Se,
A₂ is N, CH or C(R₅),
G is selected from the group consisting of:
—C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, —C₁-C₆alkyl optionally substituted by a hydroxyl group, a halogen, —NO₂, and —CN, in which:
R_{G1} and R_{G2} at each occurrence are each independently selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂-C₆alkynyl, a C₃-C₆cycloalkyl, phenyl and —(CH₂)₁₋₄-phenyl;
R_{G3} is selected from the group consisting of a C₁-C₆alkyl optionally substituted by 1 to 3 halogen atoms, a C₂-C₆alkenyl, a C₂-C₆alkynyl, a C₃-C₆cycloalkyl, phenyl and —(CH₂)₁₋₄-phenyl; or R_{G1} and R_{G2}, together with the atom to which each is attached are combined to form a C₃-C₈heterocycloalkyl; or in the alternative, G is selected from the group consisting of: 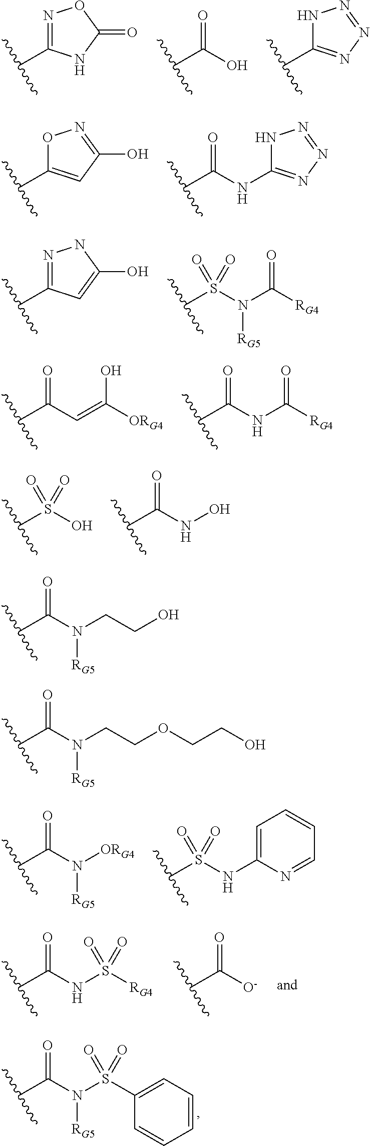
wherein R_{G4} is selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂-C₆alkynyl and a C₃-C₆cycloalkyl, and R_{G5} represents a hydrogen atom or a C₁-C₆alkyl group optionally substituted by 1 to 3 halogen atoms,
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a C₂-C₆alkenyl; a C₂-C₆alkynyl; a halogen; and —CN,
R₆ represents a group selected from the group consisting of:
hydrogen;
a linear or branched —C₁-C₆alkylene-R₈ group;
a —C₂-C₆alkenyl;
—X₂—O—R₇; 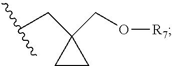
—X₂—NSO₂—R₇;
—C═C(R₉)—Y, —O—R₇;
a C₃-C₆cycloalkyl;
a C₃-C₆heterocycloalkyl optionally substituted by a hydroxyl group;
a C₃-C₆cycloalkylene-Y₂—R₇;
a C₃-C₆heterocycloalkylene-Y₂—R₇ group, and
a heteroarylene-R₇ group optionally substituted by a linear or branched C₁-C₆alkyl group,
R₇ represents a group selected from the group consisting of: a linear or branched C₁-C₆alkyl group; a (C₃-C₆)cycloalkylene-R₈; 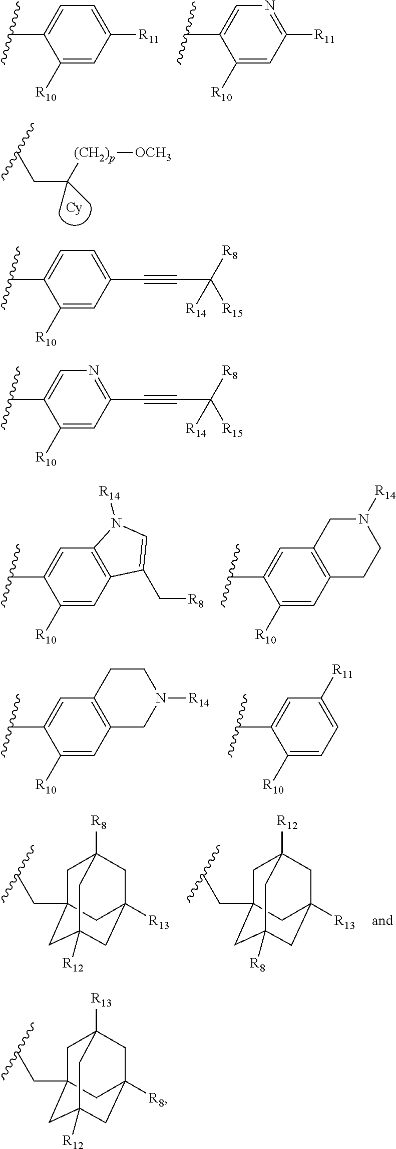
wherein Cy represents a C₃-C₈cycloalkyl,
R₈ represents a group selected from the group consisting of: hydrogen; a linear or branched C₁-C₆alkyl, —NR′ₐR′_{b}; —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}; —X′₂—NR′ₐR′_{b}; —NR′_{c}—X′₂—N₃ and
—NR′_{c}—X′₂-≡CH,
R₉ represents a group selected from the group consisting of a linear or branched C₁-C₆alkyl, trifluoromethyl, hydroxyl, halogen, and a C₁-C₆alkoxy,
R₁₀ represents a group selected from the group consisting of hydrogen, fluorine, chlorine, bromine, —CF₃ and methyl,
R₁₁ represents a group selected from the group consisting of hydrogen, a C₁-C₃alkylene-R₈, a —O—C₁-C₃alkylene-R₈, —CO—NRₕRᵢ and a —CH═CH—C₁-C₄alkylene-NRₕRᵢ, —CH═CH—CHO, a C₃-C₈cycloalkylene-CH₂—R₈, and a C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ and X₂ independently of one another, represent a linear or branched C₁-C₆alkylene group optionally substituted by one or two groups selected from the group consisting of trifluoromethyl, hydroxyl, a halogen, and a C₁-C₆alkoxy,
X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O⁻; a C₁-C₆alkylene-COOH; a C₁-C₆alkylene-PO(OH)₂; a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy group; and the group: 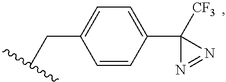 [SMILES: *Cc1ccc(C2(C(F)(F)F)N=N2)cc1]
or R′ₐ and R′_{b} form with the nitrogen atom carrying them a cycle B₃,
or R′ₐ, R′_{b} and R′_{c} form with the nitrogen atom carrying them a bridged C₃-C₈ hetero cycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C_{8 D}heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, or —NH—SO₂—,
m=0, 1 or 2,
B₁, B₂, B₃ and B₄, independently of one another, represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl,
wherein one of the R₃ and R₈ groups, if present, is covalently attached to the linker, and wherein the valency of an atom is not exceeded by virtue of one or more substituents bonded thereto; or 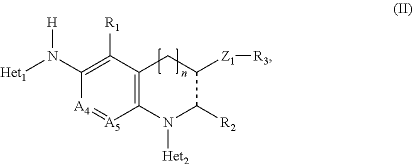
or an enantiomer, a diastereoisomer, and/or a pharmaceutically acceptable salt of any one of the foregoing, wherein:
n=0, 1 or 2,
------ represents a single or a double bond,
A₄ and A₅ independently of one another represent a carbon or a nitrogen atom,
Z₁ represents a bond, —N(R)—, or —O—, wherein R represents a hydrogen or a linear or branched C₁-C₆alkyl,
R₁ represents a group selected from the group consisting of: hydrogen; a linear or branched C₁-C₆alkyl optionally substituted by a hydroxyl or a C₁-C₆alkoxy group; a C₃-C₆cycloalkyl; trifluoromethyl; and a linear or branched C₁-C₆alkylene-heterocycloalkyl wherein the heterocycloalkyl group is optionally substituted by a linear or branched C₁-C₆alkyl group;
R₂ represents a hydrogen or a methyl;
R₃ represents a group selected from the group consisting of: hydrogen; a linear or branched C₁-C₄alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:
X₁-≡CH,
Rₐ and R_{b} independently of one another represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl groups; a C₁-C₆alkylene-SO₂OH; a C₁-C₆alkylene-SO₂O⁻; a C₁-C₆alkylene-COOH; a C₁-C₆alkylene-PO(OH)₂; a C₁-C₆alkylene-NR_{d}Rₑ; a C₁-C₆alkylene-N⁺R_{d}RₑR_{f}; a C₁-C₆alkylene-phenyl wherein the phenyl may be substituted by a C₁-C₆alkoxy group; and the group: 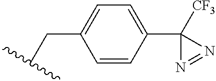 [SMILES: *Cc1ccc(C2(C(F)(F)F)N=N2)cc1]
or Rₐ and R_{b} form with the nitrogen atom carrying them a cycle B₁;
or Rₐ, R_{b} and R_{c} form with the nitrogen atom carrying them a bridged C₃-C₈ heterocycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a a cycle B₂,
or R_{d}, Rₑ and R_{f} form with the nitrogen atom carrying them a bridged C₃-C₈ heterocycloalkyl,
Het₁ represents a group selected from the group consisting of: 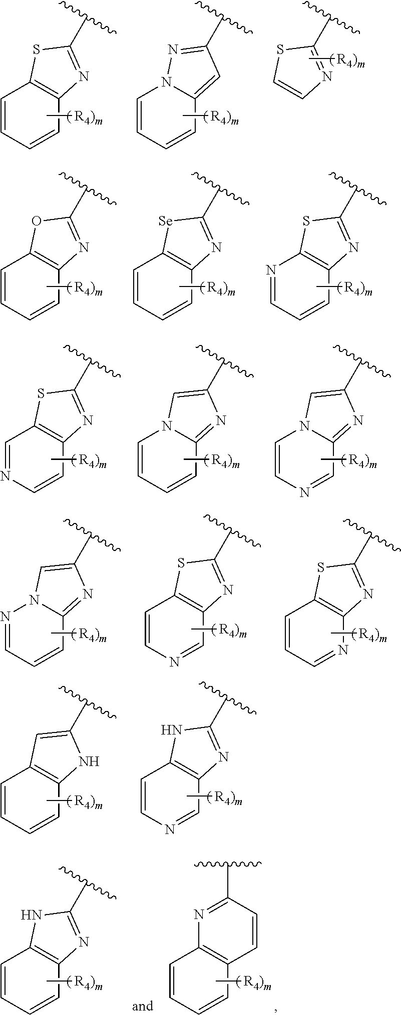
Het₂ represents a group selected from the group consisting of: 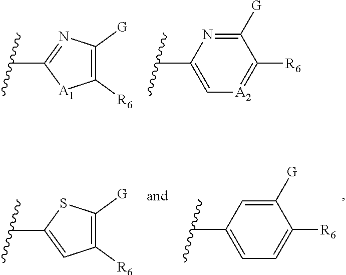
A₁ is —NH—, —N(C₁-C₃alkyl), O, S or Se,
A₂ is N, CH or C(R₅),
G is selected from the group consisting of:
—C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, —C₁-C₆alkyl optionally substituted by a hydroxyl group, halogen, —NO₂, and —CN, in which:
R_{G1} and R_{G2} at each occurrence are each independently selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂-C₆ alkynyl, a C₃-C₆cycloalkyl, phenyl and —(CH₂)₁₋₄-phenyl;
R_{G3} is selected from the group consisting of a C₁-C₆alkyl optionally substituted by 1 to 3 halogen atoms, a C₂-C₆alkenyl, a C₂-C₆alkynyl, a C₃-C₆cycloalkyl, phenyl and —(CH₂)₁₋₄-phenyl; or R_{G1} and R_{G2}, together with the atom to which each is attached are combined to form a C₃-C₈heterocycloalkyl; or in the alternative, G is selected from the group consisting of: 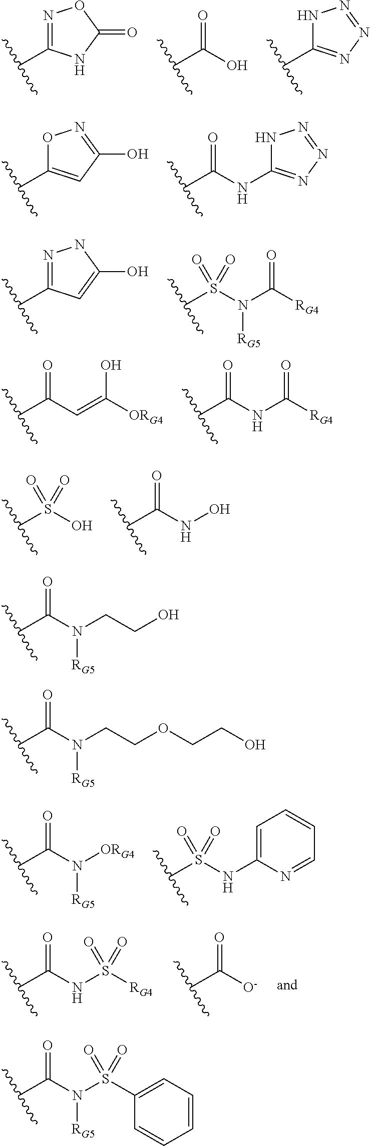
wherein R_{G4} is selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆ alkenyl, a C₂-C₆alkynyl and a C₃-C₆cycloalkyl, and R_{G5} represents a hydrogen atom or a C₁-C₆alkyl group optionally substituted by 1 to 3 halogen atoms,
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a C₂-C₆alkenyl; a C₂-C₆alkynyl; a halogen; and —CN,
R₆ represents a group selected from the group consisting of:
hydrogen;
a linear or branched —C₁-C₆alkylene-R₈ group;
a —C₂-C₆alkenyl;
—X₂—O—R₇; 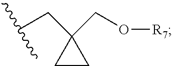
—X₂—NSO₂—R₇;
—C═C(R₉)—Y₁—O—R₇;
a C₃-C₆cycloalkyl;
a C₃-C₆heterocycloalkyl optionally substituted by a hydroxyl group;
a C₃-C₆cycloalkylene-Y₂—R₇;
a C₃-C₆heterocycloalkylene-Y₂—R₇ group, and
a heteroarylene-R₇ group optionally substituted by a linear or branched C₁-C₆alkyl group,
R₇ represents a group selected from the group consisting of: a linear or branched C₁-C₆alkyl group; a (C₃-C₆)cycloalkylene-R₈; 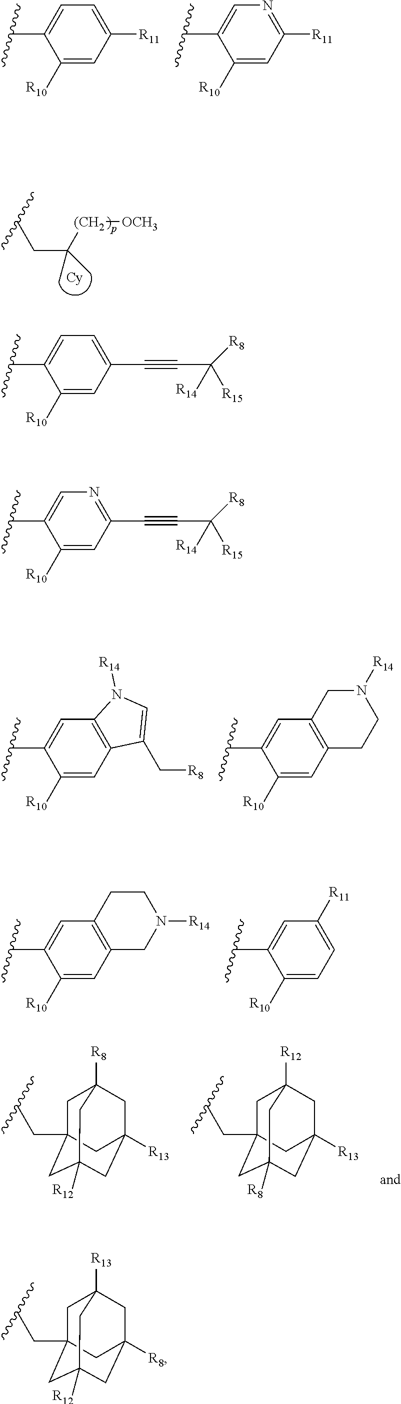
wherein Cy represents a C₃-C₈cycloalkyl,
R₈ represents a group selected from the group consisting of: hydrogen; a linear or branched C₁-C₆alkyl, —NR′ₐR′_{b}; —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}, —X′₂—NR′ₐR′_{b}, —NR′_{c}—X′₂—N₃ and
—NR′_{c}—X′₂-≡CH,
R₉ represents a group selected from the group consisting of a linear or branched C₁-C₆alkyl, trifluoromethyl, hydroxyl, a halogen, and a C₁-C₆alkoxy,
R₁₀ represents a group selected from the group consisting of hydrogen, fluorine, chlorine, bromine, —CF₃ and methyl,
R₁₁ represents a group selected from the group consisting of hydrogen, a halogen, a C₁-C₃alkylene-R₈, a —O—C₁-C₃alkylene-R₈, —CO—NRₕRᵢ and a —CH═CH—C₁-C₄alkylene-NRₕRᵢ, —CH═CH—CHO, a C₃-C₈cycloalkylene-CH₂—R₈, and a C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ represents a linear or branched C₁-C₄alkylene group optionally substituted by one or two groups selected from the group consisting of trifluoromethyl, hydroxyl, a halogen, and a C₁-C₆alkoxy,
X₂ represents a linear or branched C₁-C₆alkylene group optionally substituted by one or two groups selected from the group consisting of trifluoromethyl, hydroxyl, a halogen, and a C₁-C₆alkoxy,
X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O⁻; a C₁-C₆alkylene-COOH; a C₁-C₆alkylene-PO(OH)₂;
a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy group; and the group: 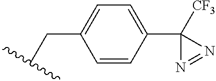 [SMILES: *Cc1ccc(C2(C(F)(F)F)N=N2)cc1]
or R′ₐ and R′_{b} form with the nitrogen atom carrying them a cycle B₃, or R′ₐ, R′_{b} and R′_{c} form with the nitrogen atom carrying them a bridged C₃-C₈ heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈ heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, or —NH—SO₂—,
m=0, 1 or 2,
B₁, B₂, B₃ and B₄, independently of one another, represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl,
wherein one of the R₃, R₈ and G groups, if present, is covalently attached to the linker, and wherein the valency of an atom is not exceeded by virtue of one or more substituents bonded thereto.

2. The compound of claim 1, or an enantiomer, a diastereoisomer, and/or a pharmaceutically acceptable salt of any one of the foregoing, wherein D is a Bcl-xL inhibitor compound of Formula (I) or Formula (II) covalently attached to the linker L: 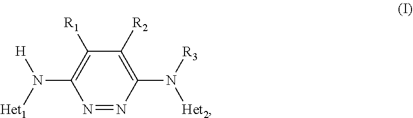
or an enantiomer, a diastereoisomer, and/or a pharmaceutically acceptable salt of any one of the foregoing, wherein:
R₁ and R₂ independently of one another represent a group selected from the group consisting of: hydrogen; a linear or branched C₁-C₆alkyl optionally substituted by a hydroxyl or a C₁-C₆alkoxy group; a C₃-C₆cycloalkyl; a trifluoromethyl; and a linear or branched C₁-C₆alkylene-heterocycloalkyl wherein the heterocycloalkyl group is optionally substituted by a linear or branched C₁-C₆alkyl group;
or R₁ and R₂ form with the carbon atoms carrying them a C₃-C₆cycloalkylene group,
R₃ represents a group selected from the group consisting of: hydrogen; a C₃-C₆cycloalkyl; a linear or branched C₁-C₆alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:
—X₁-≡CH,
Rₐ and R_{b} independently of one another represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl groups; a C₁-C₆alkylene-SO₂OH; a C₁-C₆alkylene-SO₂O⁻; a C₁-C₆alkylene-COOH; a C₁-C₆alkylene-PO(OH)₂; a C₁-C₆alkylene-NR_{d}Rₑ; a C₁-C₆alkylene-N⁺R_{d}RₑR_{f}; a C₁-C₆alkylene-phenyl wherein the phenyl may be substituted by a C₁-C₆alkoxy group; and the group: 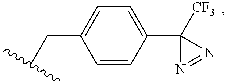 [SMILES: *Cc1ccc(C2(C(F)(F)F)N=N2)cc1]
or Rₐ and R_{b} form with the nitrogen atom carrying them a cycle B₁;
or Rₐ, R_{b} and R_{c} form with the nitrogen atom carrying them a bridged C₃-C₆hetero cycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a cycle B₂,
or R_{d}, Rₑ and R_{f} form with the nitrogen atom carrying them a bridged C₃-C₆hetero cycloalkyl,
Het₁ represents a group selected from the group consisting of: 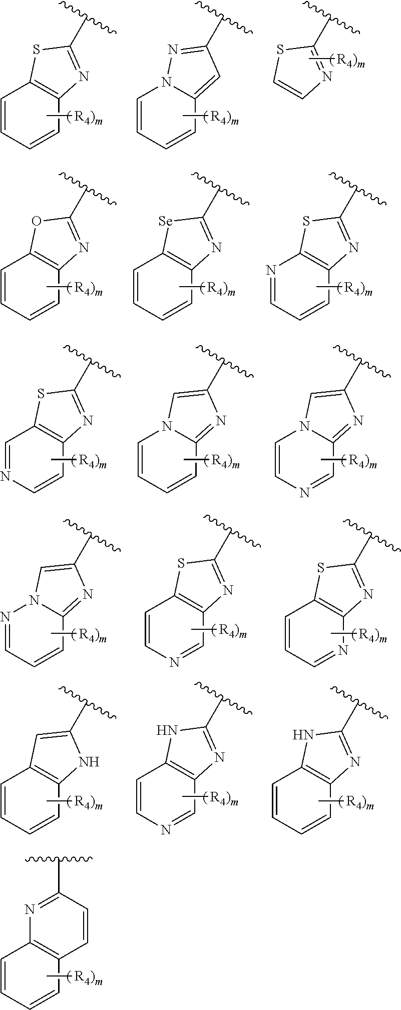
Het₂ represents a group selected from the group consisting of: 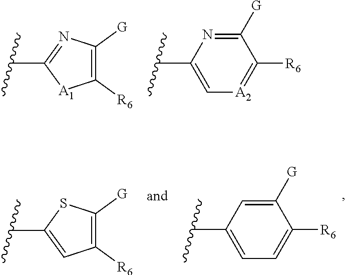
A₁ is —NH—, —N(C₁-C₃alkyl), O, S or e,
A₂ is N, CH or C(R₅),
G is selected from the group consisting of:
—C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, a halogen, —NO₂, and —CN, in which:
R_{G1} and R_{G2} at each occurrence are each independently selected from the group consisting of hydrogen, a C₁-C₆alkyl optionally substituted by 1 to 3 halogen atoms, a C₂-C₆alkenyl, a C₂-C₆alkynyl, a C₃-C₆cycloalkyl, phenyl and —(CH₂)₁₋₄-phenyl;
R_{G3} is selected from the group consisting of a C₁-C₆alkyl optionally substituted by 1 to 3 halogen atoms, a C₂-C₆alkenyl, a C₂-C₆alkynyl, a C₃-C₆cycloalkyl, phenyl and —(CH₂)₁₋₄-phenyl; or R_{G1} and R_{G2}, together with the atom to which each is attached are combined to form a C₃-C₆heterocycloalkyl; or in the alternative, G is selected from the group consisting of: 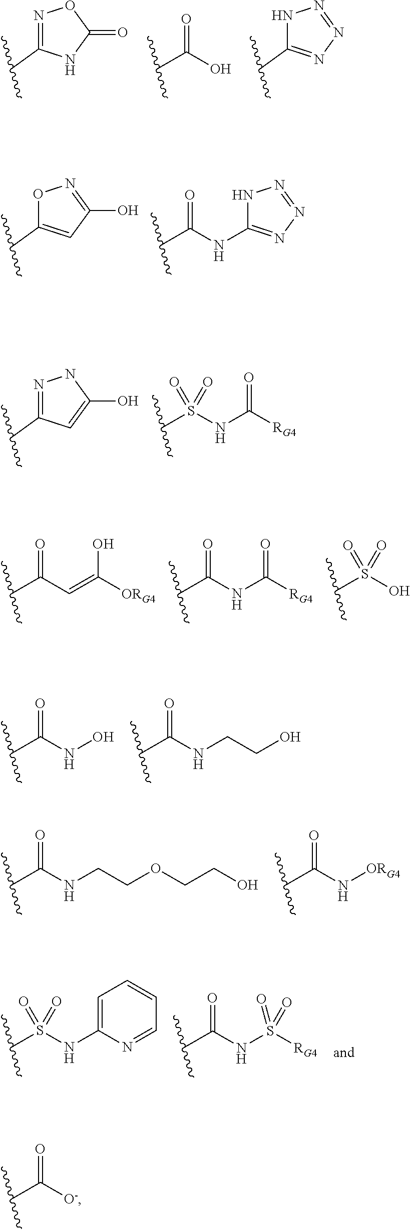
wherein R_{G4} is selected from the group consisting of a C₁-C₆alkyl optionally substituted by 1 to 3 halogen atoms, a C₂-C₆alkenyl, a C₂-C₆alkynyl and a C₃-C₆cycloalkyl,
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a C₂-C₆alkenyl; a C₂-C₆alkynyl; a halogen; and —CN,
R₆ represents a group selected from the group consisting of:
hydrogen;
a linear or branched —C₁-C₆alkylene-R₃ group;
a —C₂-C₆alkenyl;
—X₂—O—R₇ 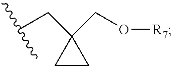
—X₂—NSO₂—R₇;
—C═C(R₉)—Y₁—O—R₇;
a C₃-C₆cycloalkyl;
a C₃-C₆heterocycloalkyl optionally substituted by a hydroxyl group;
a C₃-C₆cycloalkylene-Y₂—R₇;
a C₃-C₆heterocycloalkylene-Y₂—R₇ group, and a heteroarylene-R₇ group optionally substituted by a linear or branched C₁-C₆alkyl group,
R₇ represents a group selected from the group consisting of: a linear or branched C₁-C₆alkyl group; a (C₃-C₆)cycloalkylene-R₈; 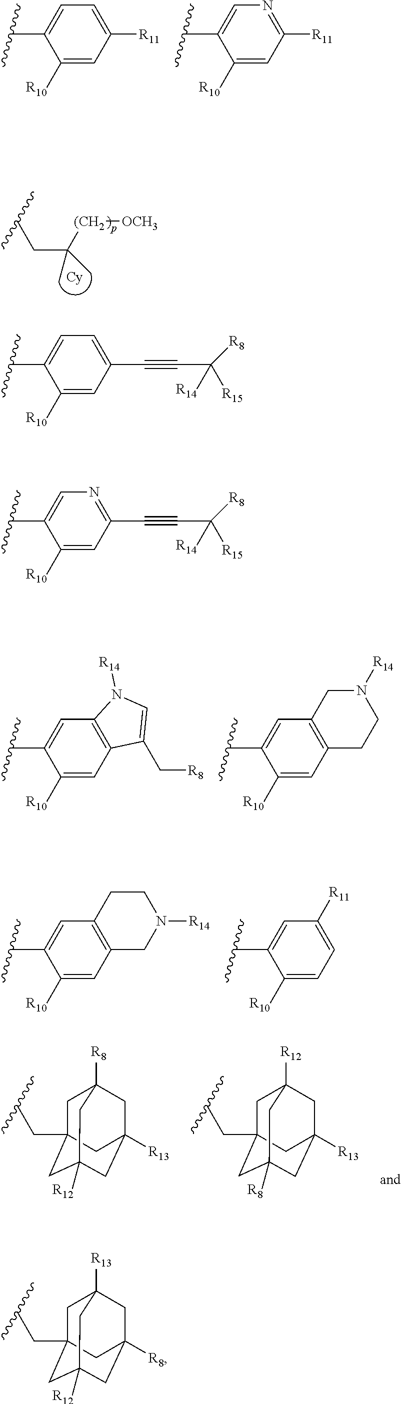
wherein Cy represents a C₃-C₈cycloalkyl,
R₈ represents a group selected from the group consisting of: hydrogen; a linear or branched C₁-C₆alkyl, —NR′ₐR′_{b}; —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}; —X′₂—NR′ₐR′_{b}; —NR′_{c}—X′₂—N₃ and —NR′_{c}—X′₂-≡CH,
R₉ represents a group selected from the group consisting of a linear or branched C₁-C₆alkyl, trifluoromethyl, hydroxyl, halogen, and a C₁-C₆alkoxy,
R₁₀ represents a group selected from the group consisting of hydrogen, fluorine, chlorine, bromine, —CF₃ and methyl,
R₁₁ represents a group selected from the group consisting of hydrogen, a C₁-C₃alkylene-R₈, a —O—C₁-C₃alkylene-R₈, —CO—NRₕRᵢ and a —CH═CH—C₁-C₄alkylene-NRₕRᵢ, —CH═CH—CHO, a C₃-C₈cycloalkylene-CH₂—R₈, and a C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ and X₂ independently of one another, represent a linear or branched C₁-C₆alkylene group optionally substituted by one or two groups selected from the group consisting of trifluoromethyl, hydroxyl, a halogen, and a C₁-C₆alkoxy,
X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O⁻; a C₁-C₆alkylene-COOH; a C₁-C₆alkylene-PO(OH)₂; a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy
group; and the group: 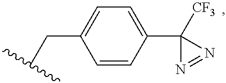 [SMILES: *Cc1ccc(C2(C(F)(F)F)N=N2)cc1]
or R′ₐ and R′_{b} form with the nitrogen atom carrying them a cycle B₃,
or R′ₐ, R′_{b} and R′_{c} form with the nitrogen atom carrying them a bridged C₃-C₈ hetero cycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈ heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, or —NH—SO₂—,
m=0, 1 or 2,
B₁, B₂, B₃ and B₄, independently of one another, represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl,
wherein one of the R₃ and R₈ groups, if present, is covalently attached to the linker, and wherein the valency of an atom is not exceeded by virtue of one or more substituents bonded thereto; or 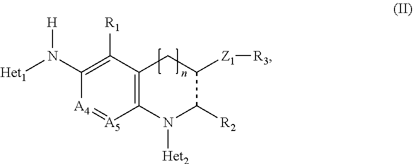
or an enantiomer, a diastereoisomer, and/or a pharmaceutically acceptable salt of any one of the foregoing, wherein:
n=0, 1 or 2,
----- represents a single or a double bond,
A₄ and A₅ independently of one another represent a carbon or a nitrogen atom,
Z₁ represents a bond, —N(R)—, or —O—, wherein R represents a hydrogen or a linear or branched C₁-C₆alkyl,
R₁ represents a group selected from the group consisting of: hydrogen; a linear or branched C₁-C₆alkyl optionally substituted by a hydroxyl or a C₁-C₆alkoxy group; a C₃-C₆cycloalkyl; trifluoromethyl; and a linear or branched C₁-C₆alkylene-heterocycloalkyl wherein the heterocycloalkyl group is optionally substituted by a linear or branched C₁-C₆alkyl group;
R₂ represents a hydrogen or a methyl;
R₃ represents a group selected from the group consisting of: hydrogen; a linear or branched C₁-C₄alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:
—X₁-≡CH,
Rₐ and R_{b} independently of one another represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl groups; a C₁-C₆alkylene-SO₂OH; a C₁-C₆alkylene-SO₂O⁻; a C₁-C₆alkylene-COOH; a C₁-C₆alkylene-PO(OH)₂; a C₁-C₆alkylene-NR_{d}Rₑ; a C₁-C₆alkylene-N⁺R_{d}RₑR_{f}; a C₁-C₆alkylene-phenyl wherein the phenyl may be substituted by a C₁-C₆alkoxy group; and the group: 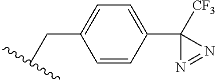 [SMILES: *Cc1ccc(C2(C(F)(F)F)N=N2)cc1]
or Rₐ and R_{b} form with the nitrogen atom carrying them a cycle B₁;
or Rₐ, R_{b} and R_{c} form with the nitrogen atom carrying them a bridged C₃-C₈ heterocycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a a cycle B₂,
or R_{d}, Rₑ and R_{f} form with the nitrogen atom carrying them a bridged C₃-C₈ heterocycloalkyl,
Het₁ represents a group selected from the group consisting of: 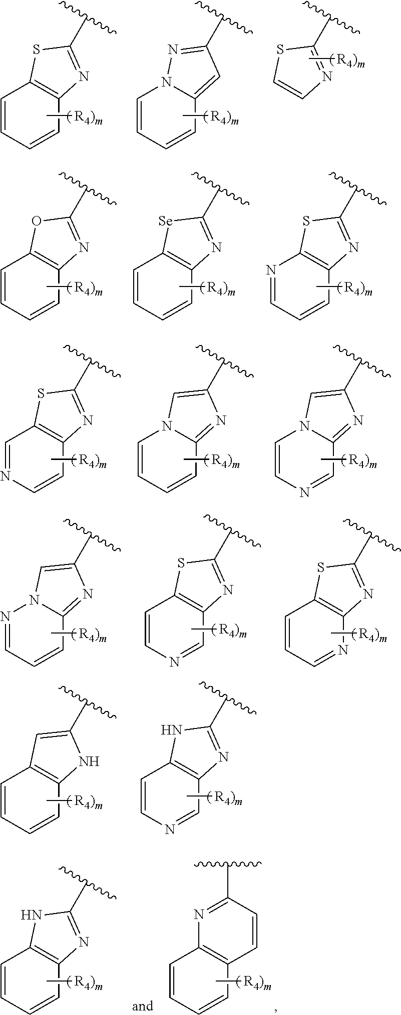
Het₂ represents a group selected from the group consisting of: 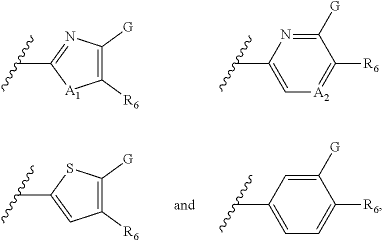
A₁ is —NH—, —N(C₁-C₃alkyl), O, S or Se,
A₂ is N, CH or C(R₅),
G is selected from the group consisting of:
—C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, halogen, —NO₂, and —CN, in which:
R_{G1} and R_{G2} at each occurrence are each independently selected from the group consisting of hydrogen, a C₁-C₆alkyl optionally substituted by 1 to 3 halogen atoms, a C₂-C₆alkenyl, a C₂-C₆ alkynyl, a C₃-C₆cycloalkyl, phenyl and —(CH₂)₁₋₄-phenyl;
R_{G3} is selected from the group consisting of a C₁-C₆alkyl optionally substituted by 1 to 3 halogen atoms, a C₂-C₆alkenyl, a C₂-C₆alkynyl, a C₃-C₆cycloalkyl, phenyl and —(CH₂)₁₋₄-phenyl; or R_{G1} and R_{G2}, together with the atom to which each is attached are combined to form a C₃-C₆heterocycloalkyl; or in the alternative, G is selected from the group consisting of: 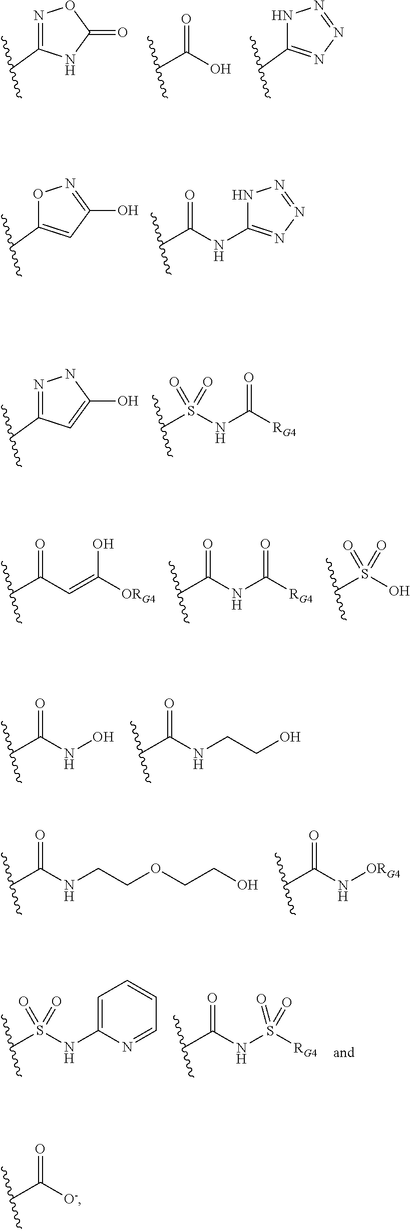
wherein R_{G4} is selected from the group consisting of a C₁-C₆alkyl optionally substituted by 1 to 3 halogen atoms, a C₂—C alkenyl, a C₂-C₆alkynyl and a C₃-C₆cycloalkyl,
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a C₂-C₆alkenyl; a C₂-C₆alkynyl; a halogen; and —CN,
R₆ represents a group selected from the group consisting of:
hydrogen;
a linear or branched —C₁-C₆alkylene-R₈ group;
a —C₂-C₆alkenyl;
—X₂—O—R₇; 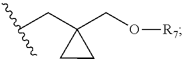
—X₂—NSO₂—R₇;
—C═C(R₉)—Y₁—O—R₇;
a C₃-C₆cycloalkyl;
a C₃-C₆heterocycloalkyl optionally substituted by a hydroxyl group;
a C₃-C₆cycloalkylene-Y₂—R₇;
a C₃-C₆heterocycloalkylene-Y₂—R₇ group, and
a heteroarylene-R₇ group optionally substituted by a linear or branched C₁-C₆alkyl group,
R₇ represents a group selected from the group consisting of: a linear or branched C₁-C₆alkyl group; a (C₃-C₆)cycloalkylene-R₈; 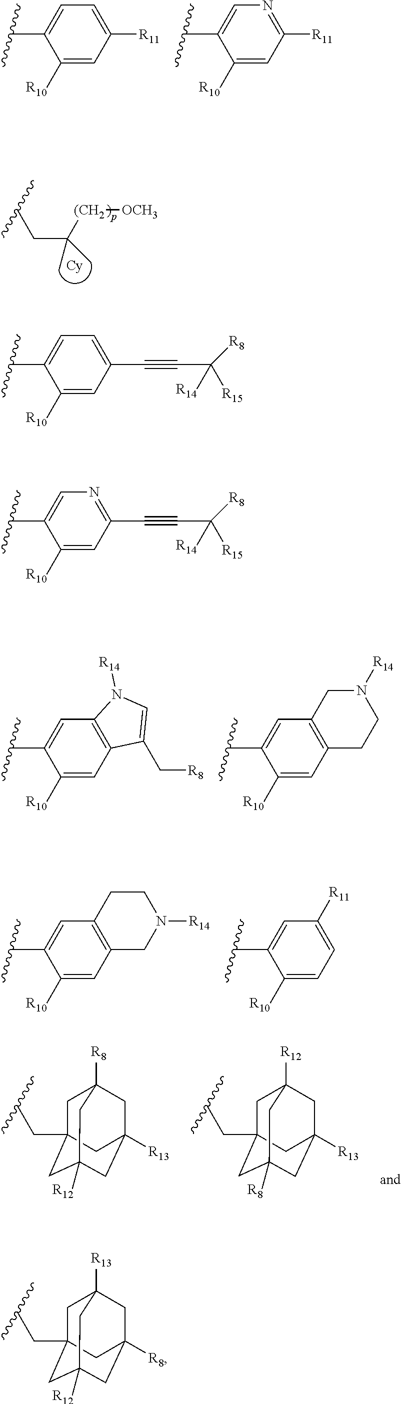
wherein Cy represents a C₃-C₈cycloalkyl,
R₈ represents a group selected from the group consisting of: hydrogen; a linear or branched C₁-C₆ alkyl, —NR′ₐR′_{b}; —NR′ₐ—CO—OR′_{b}; —NR′ₐ—CO—R′_{c}; —N⁺R′ₐR′_{b}R′_{b}; —O—R′_{c}; —NH—X′₂—N⁺R′ₐR′_{b}R′_{c}; —O—X′₂—NR′ₐR′_{b}, —X′₂—NR′ₐR′_{b}, —NR′_{c}—X′₂—N₃ and:
—NR′_{c}—X′₂-≡CH,
R₉ represents a group selected from the group consisting of a linear or branched C₁-C₆ alkyl, trifluoromethyl, hydroxyl, a halogen, and a C₁-C₆ alkoxy,
R₁₀ represents a group selected from the group consisting of hydrogen, fluorine, chlorine, bromine, —CF₃ and methyl,
R₁₁ represents a group selected from the group consisting of hydrogen, a halogen, a C₁-C₃ alkylene-R₈, a —O—C₁-C₃ alkylene-R₈, —CO—NRₕRᵢ and a —CH═CH—C₁-C₄alkylene-NRₕRᵢ, —CH═CH—CHO, a C₃-C₈cycloalkylene-CH₂—R₈, and a C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ represents a linear or branched C₁-C₄alkylene group optionally substituted by one or two groups selected from the group consisting of trifluoromethyl, hydroxyl, a halogen, and a C₁-C₆alkoxy,
X₂ represents a linear or branched C₁-C₆alkylene group optionally substituted by one or two groups selected from the group consisting of trifluoromethyl, hydroxyl, a halogen, and a C₁-C₆alkoxy,
X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O⁻; a C₁-C₆alkylene-COOH; a C₁-C₆alkylene-PO(OH)₂; a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy group; and the group: 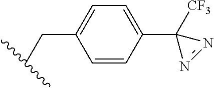 [SMILES: *Cc1ccc(C2(C(F)(F)F)N=N2)cc1]
or R′ₐ and R′_{b} form with the nitrogen atom carrying them a cycle B₃,
or R′ₐ, R′_{b} and R′_{c} form with the nitrogen atom carrying them a bridged C₃-C₈ heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈ heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, or —NH—SO₂—,
m=0, 1 or 2,
B₁, B₂, B₃ and B₄, independently of one another, represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl,
wherein one of the R₃ and R₈ groups, if present, is covalently attached to the linker, and wherein the valency of an atom is not exceeded by virtue of one or more substituents bonded thereto.

3. The compound of claim 1 or 2, wherein the linker L comprises at least one group selected from the group consisting of: a linear or branched C₁-C₂₀alkylene optionally substituted by one to three groups selected from the group consisting of a C₁-C₈alkyl, a C₃-C₈cycloalkyl, trifluoromethyl, hydroxyl, a halogen, and a C₁-C₆alkoxy; a C₃-C₁₀cycloalkylene; a C₃-C₆heterocycloalkylene; —C(O)—; —O—; —S—; —N(R₁₆)—; —N(R₁₆)—C(O)—; —C(O)—N(R₁₆)—; —CH₂—C(O)—N(R₁₆)—; —N(R₁₆)—C(O)—CH₂—; a polyoxyethylene (PEG) group; an arylene group optionally substituted by one or two groups selected from the group consisting of a C₁-C₈alkyl, trifluoromethyl, hydroxyl, a halogen, and a C₁-C₆alkoxy; and a heteroarylene group, wherein R₁₆ represents hydrogen or C₁-C₆alkyl.

4. The compound of claim 1 or 2, wherein the linker L comprises at least one group selected from the group consisting of: a linear or branched C₁-C₂₀alkylene optionally substituted by one or two groups selected from the group consisting of a C₁-C₈alkyl, a C₃-C₈cycloalkyl, trifluoromethyl, hydroxyl, a halogen, and a C₁-C₆alkoxy; a C₃-C₁₀cycloalkylene; —C(O)—; —O—; —S—; —N(R₁₆)—; —N(R₁₆)—C(O)—; —C(O)—N(R₁₆)—; —CH₂—C(O)—N(R₁₆)—; —N(R₁₆)—C(O)—CH₂—; a polyoxyethylene (PEG) group; an arylene group optionally substituted by one or two groups selected from the group consisting of a C₁-C₆alkyl, trifluoromethyl, hydroxyl, a halogen, and a C₁-C₆alkoxy; and a heteroarylene group, wherein R₁₆ represents hydrogen or C₁-C₆alkyl.

5. The compound of any one of claims 1-4, wherein the linker L comprises a 1,2,3-triazolene group formed by reacting an azide-containing precursor with an alkyne-containing precursor.

6. The compound of any one of claims 1-4, wherein -L- is represented by formula (i), (ii), (iii), (iv), (v), (vi) or (vii): 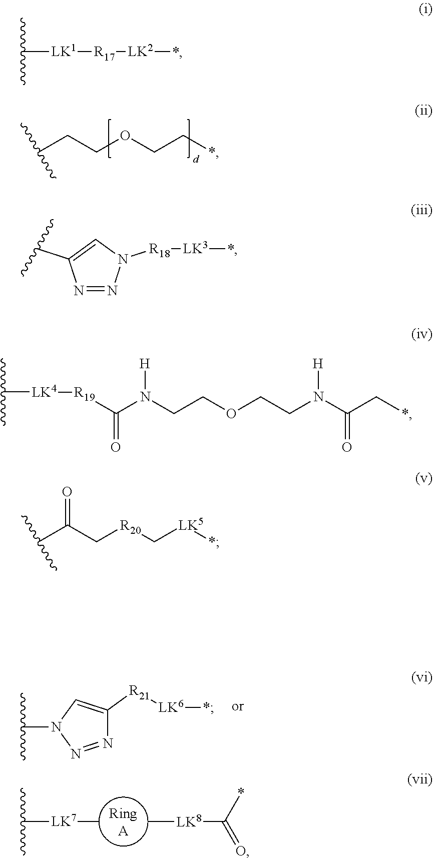
wherein:
LK¹ is a bond, —NR¹⁶— or —C(O)—;
LK² is a bond, —C(O)— or —N(R₁₆)—C(O)—CH₂—*;
LK³ is —C(O)— or —N(R₁₆)—C(O)—CH₂—*;
LK⁴ is a bond or —C(O)—;
LK⁵ is a bond or —C(O)—;
LK⁶ is a bond, —C(O)—, —O—CH₂—C(O)—*, or —N(R₁₆)—C(O)—CH₂—*;
LK⁷ is a bond or —NR¹⁶—;
LK⁸ is a bond, —R₂₂—, —O—R₂₂— or —C(O)—R₂₂—;
Ring A is a C₃-C₈ heterocyloalkylene;
R₁₆ is H or methyl;
R₁₇ is a C₁-C₂₀alkylene, a C₃₋₁₀cycloalkylene, a C₃₋₁₀cycloalkylene-CH₂—**, phenylene, —C₁-C₂₀alkylene-OCH₂CH₂—**, —C₁-C₂₀alkylene-OCH₂—**, —CH₂—(OCH₂CH₂)ₚ—OCH₂—** or —(CH₂CH₂O)ₚ—(C₁-C₃alkylene)-**, wherein the C₁-C₂₀alkylene or the phenylene is optionally substituted with one or two R₁₇ₐ; and ** indicates the attachment point to LK²;
R₁₇ₐ, for each occurrence, is independently a linear or branched C₁-C₆alkyl or a halogen, or two R₁₇ₐ together with the carbon atom from which they are attached form a C₃-C₆cycloalkyl;
R₁₈ is a C₁-C₂₀alkylene or —CH₂CH₂—(OCH₂CH₂)ₚ—**, wherein ** indicates the attachment point to LK³;
R₁₉ is a C₁-C₆alkylene;
R₂₀ is a C₃-C₁₀cycloalkylene, phenylene, —S— or —N(R₁₆)—;
R₂₁ is a C₁-C₂₀alkylene or —CH₂—(OCH₂CH₂)ₚ—**, wherein ** indicates the attachment point to LK⁶;
R₂₂ is a C₁-C₆alkylene;
p is an integer from 1 to 7;
d is an integer from 1 to 7; 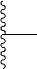
is a bond to the Bcl-xL inhibitor compound; and
-* is a bond to DSM.

7. The compound of any one of claims 1-4, wherein -L- is represented by formula (i), (ii), (iii), (iv), (v), or (vi): 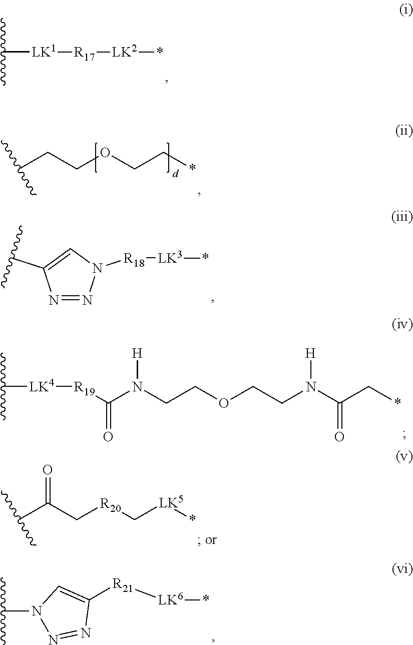
wherein:
LK¹ is a bond or —C(O)—;
LK² is a bond, —C(O)— or —N(R₁₆)—C(O)—CH₂—*;
LK³ is —C(O)— or —N(R₁₆)—C(O)—CH₂—*;
LK⁴ is a bond or —C(O)—;
LK⁵ is a bond or —C(O)—;
LK⁶ is a bond, —O—CH₂—C(O)—*, or —N(R₁₆)—C(O)—CH₂—*;
R₁₆ is H or methyl;
R₁₇ is a C₁-C₂₀alkylene, a C₃₋₁₀cycloalkylene, phenylene, or —CH₂—(OCH₂CH₂)ₚ—OCH₂—, wherein the C₁-C₂₀alkylene or the phenylene is optionally substituted with one or two R₁₇ₐ;
R₁₇ₐ, for each occurrence, is independently a linear or branched C₁₋₆alkyl or a halogen, or two R₁₇ₐ together with the carbon atom from which they are attached form a C₃₋₆ cycloalkyl;
R₁₈ is a C₁₋₂₀alkylene or —CH₂CH₂—(OCH₂CH₂)ₚ—**, wherein ** indicates the attachment point to LK³;
R₁₉ is a C₁₋₆alkylene;
R₂₀ is a C₃₋₁₀cycloalkylene, phenylene, —S— or —N(R₁₆)—;
R₂₁ is a C₁₋₂₀alkylene or —CH₂—(OCH₂CH₂)ₚ—**, wherein ** indicates the attachment point to LK⁶;
p is an integer from 1 to 7;
d is an integer from 1 to 7; 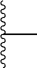
is a bond to the Bcl-xL inhibitor compound; and
-* is a bond to DSM.

8. The compound of any one of claims 1-4, wherein the linker is selected from the group consisting of: 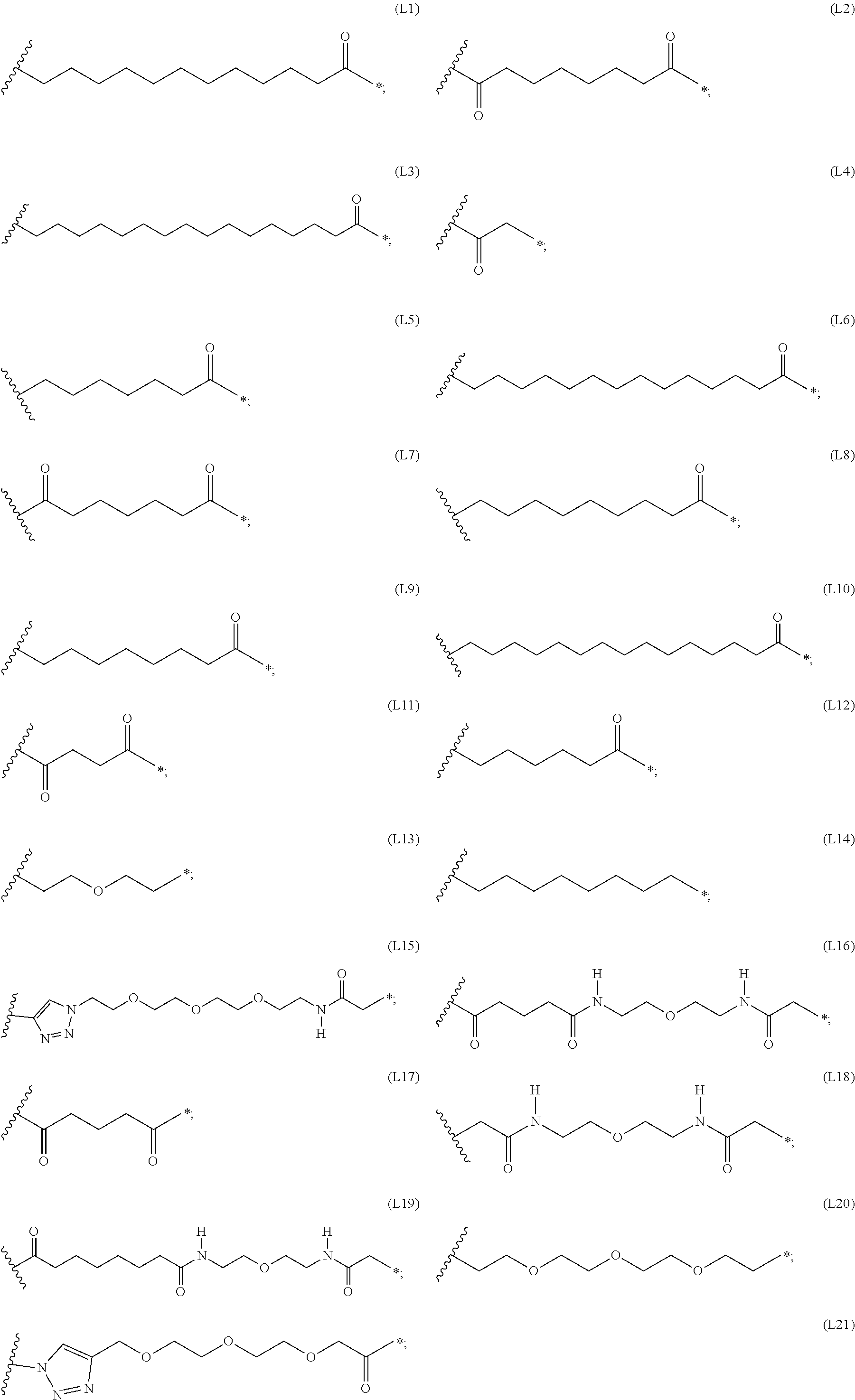 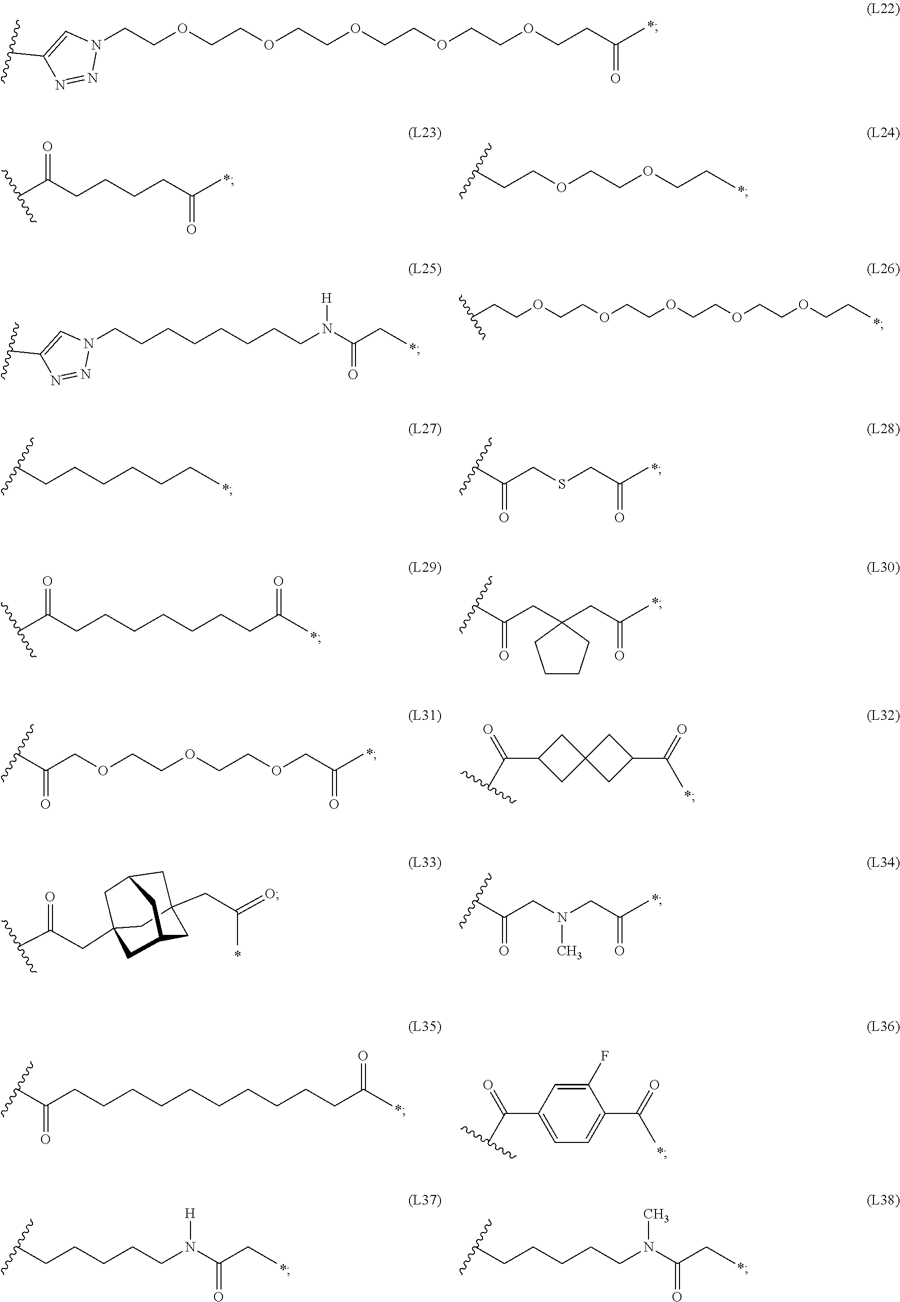 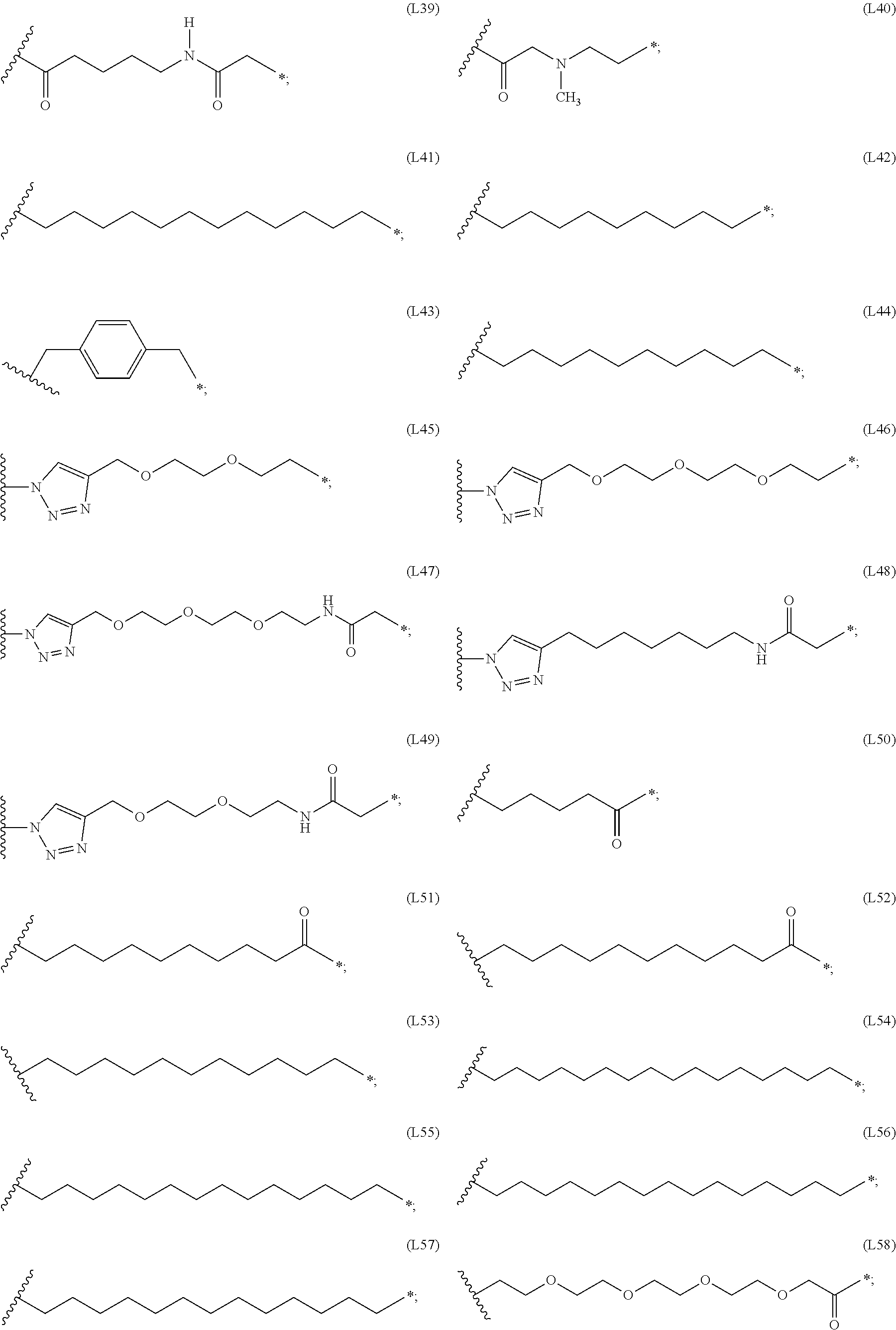 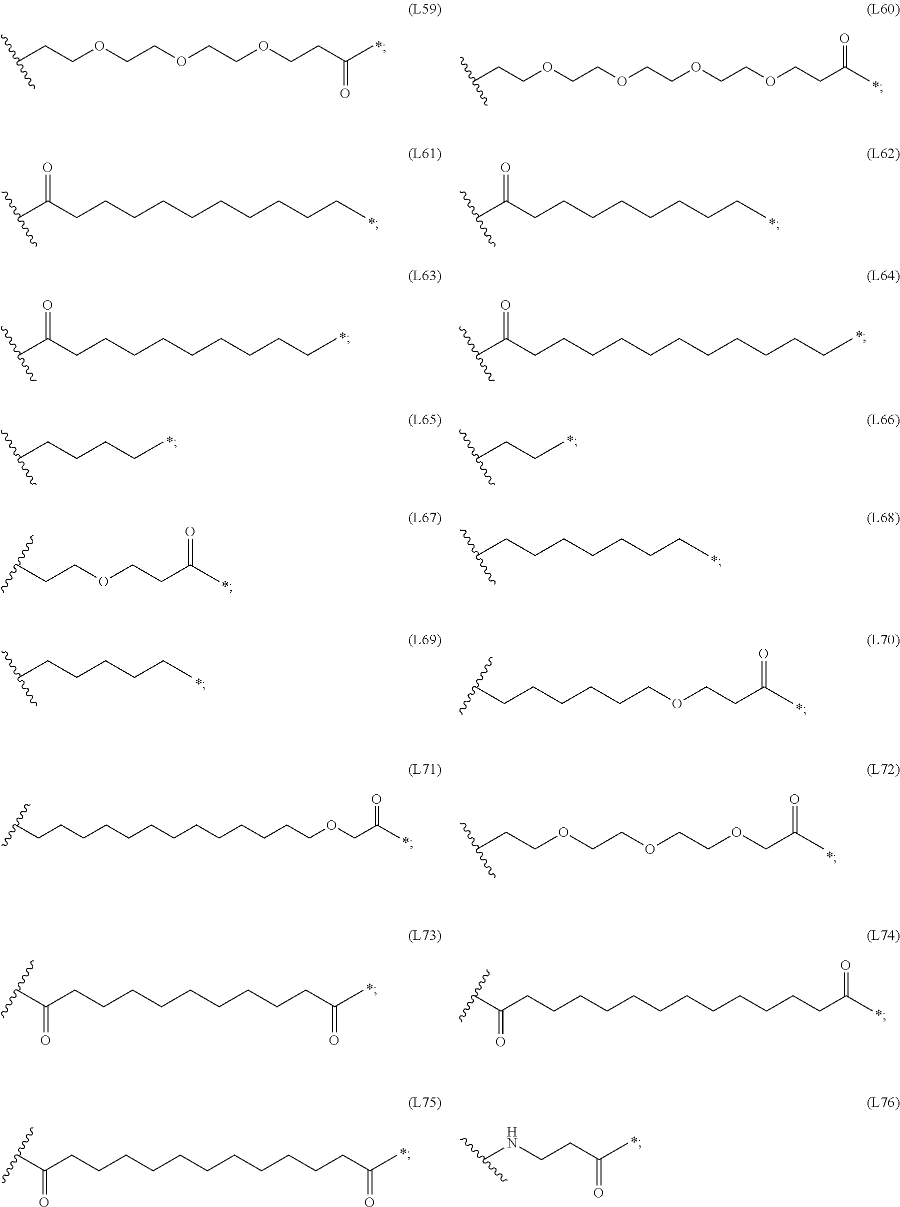 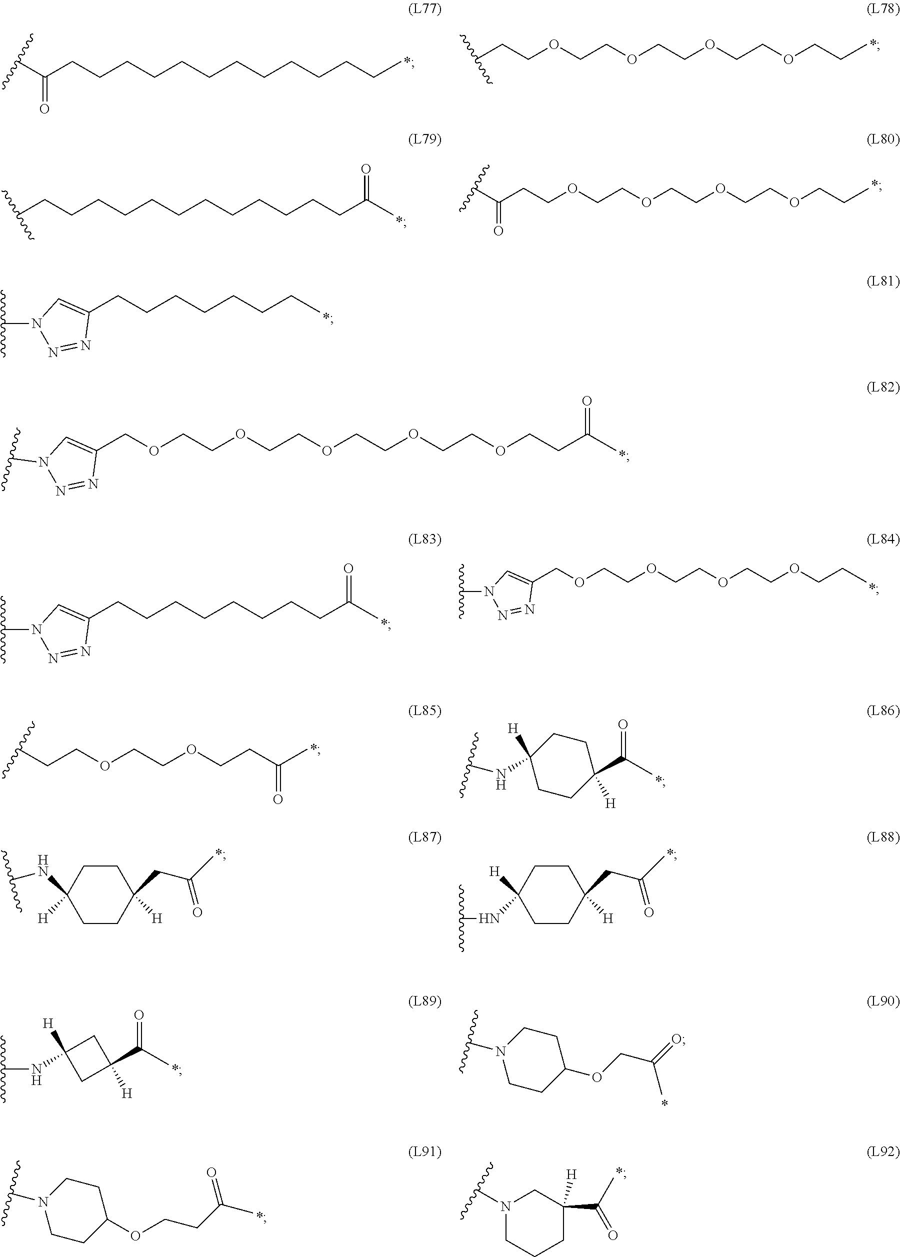 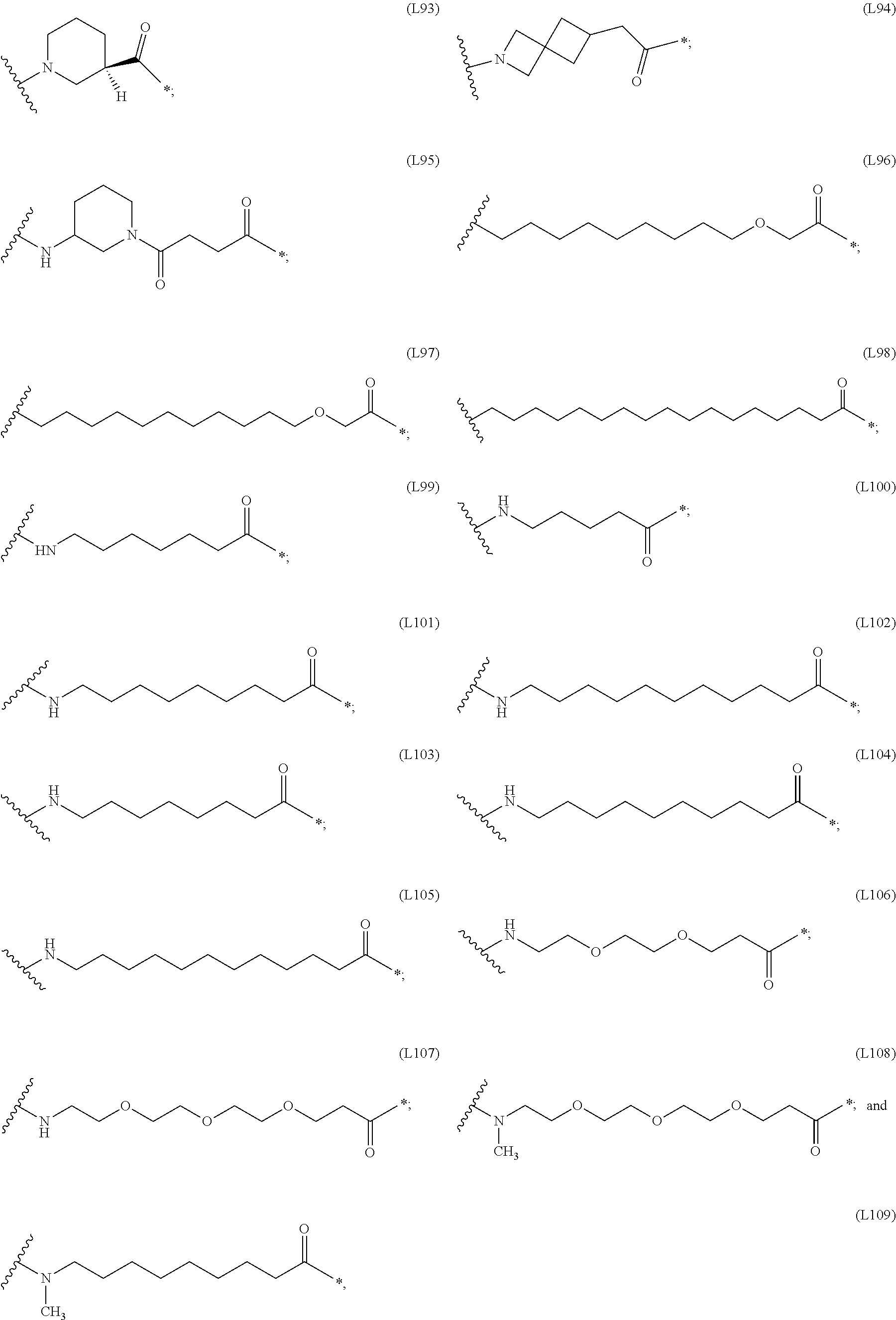
wherein: 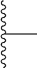
is a bond to the Bcl-xL inhibitor compound; and
-* is a bond to DSM.

9. The compound of any one of claims 1-8, wherein D comprises a compound of Formula (I): 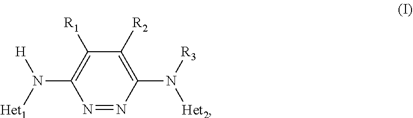
or an enantiomer, a diastereoisomer, and/or a pharmaceutically acceptable salt of any one of the foregoing, wherein:
R₁ and R₂ independently of one another represent a group selected from the group consisting of: hydrogen; a linear or branched C₁-C₆alkyl optionally substituted by a hydroxyl or a C₁-C₆alkoxy group; a C₃-C₆cycloalkyl; trifluoromethyl; and a linear or branched C₁-C₆alkylene-heterocycloalkyl wherein the heterocycloalkyl group is optionally substituted by a linear or branched C₁-C₆alkyl group;
or R₁ and R₂ form with the carbon atoms carrying them a C₃-C₆cycloalkylene group,
R₃ represents a group selected from the group consisting of: hydrogen; a C₃-C₆cycloalkyl; a linear or branched C₁-C₆alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:
—X₁-≡CH,
Rₐ and R_{b} independently of one another represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl groups; a C₁-C₆alkylene-SO₂OH; a C₁-C₆alkylene-SO₂O⁻; a C₁-C₆alkylene-COOH; a C₁-C₆alkylene-PO(OH)₂; a C₁-C₆alkylene-NR_{d}Rₑ; a C₁-C₆alkylene-N⁺R_{d}RₑR_{f}; a C₁-C₆alkylene-phenyl wherein the phenyl may be substituted by a C₁-C₆alkoxy group; and the group: 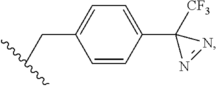 [SMILES: *Cc1ccc(C2(C(F)(F)F)N=N2)cc1]
or Rₐ and R_{b} form with the nitrogen atom carrying them a cycle B₁;
or Rₐ, R_{b} and R_{c} form with the nitrogen atom carrying them a bridged C₃-C₆hetero cycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a a cycle B₂,
or R_{d}, Rₑ and R_{f} form with the nitrogen atom carrying them a bridged C₃-C₆hetero cycloalkyl,
Het₁ represents a group selected from the group consisting of: 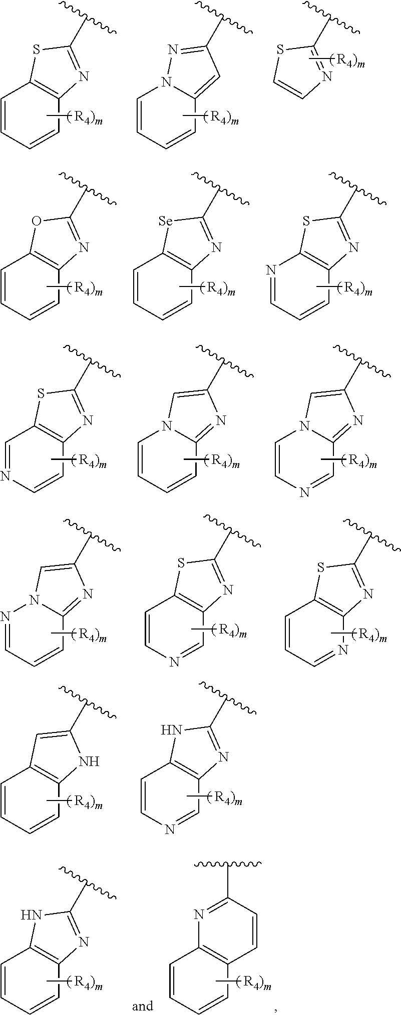
Het₂ represents a group selected from the group consisting of: 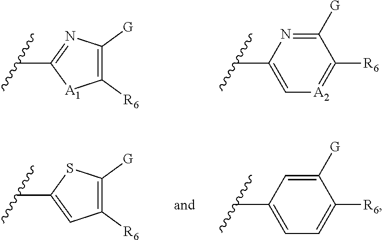
A, is —NH—, —N(C₁-C₃alkyl), O, S or Se,
A₂ is N, CH or C(R₅),
G is selected from the group consisting of:
—C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, —C₁-C₆alkyl optionally substituted by a hydroxyl group, a halogen, —NO₂, and —CN, in which:
R_{G1} and R_{G2} at each occurrence are each independently selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂-C₆alkynyl, a C₃-C₆cycloalkyl, phenyl and —(CH₂)₁₋₄-phenyl;
R_{G3} is selected from the group consisting of a C₁-C₆alkyl optionally substituted by 1 to 3 halogen atoms, a C₂-C₆alkenyl, a C₂-C₆alkynyl, a C₃-C₆cycloalkyl, phenyl and —(CH₂)₁₋₄-phenyl; or R_{G1} and R_{G2}, together with the atom to which each is attached are combined to form a C₃-C₆heterocycloalkyl; or in the alternative, G is selected from the group consisting of: 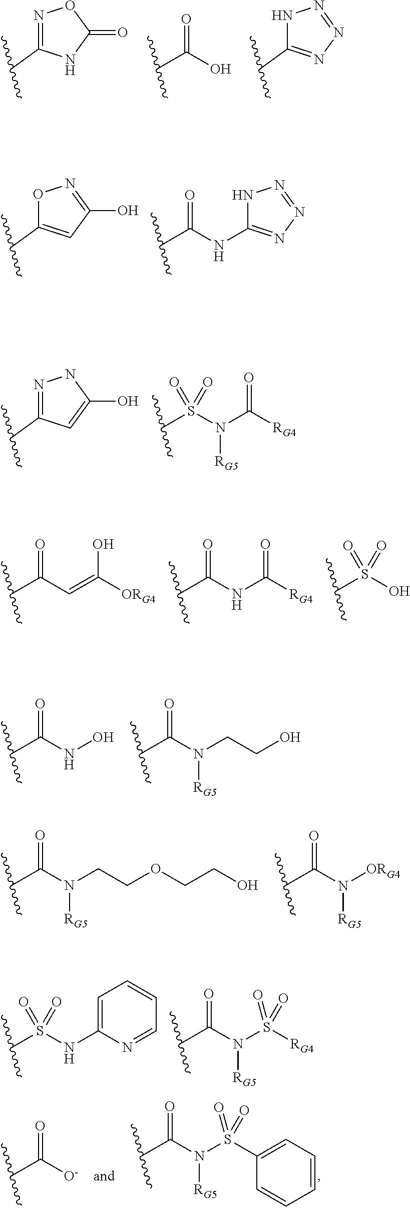
wherein R_{G4} is selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂-C₆alkynyl and a C₃-C₆cycloalkyl, and R_{G5} represents a hydrogen atom or a C₁-C₆alkyl group optionally substituted by 1 to 3 halogen atoms,
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a C₂-C₆alkenyl; a C₂-C₆alkynyl; a halogen; and —CN,
R₆ represents a group selected from the group consisting of:
hydrogen;
a linear or branched —C₁-C₆alkylene-R₈ group;
a —C₂-C₆alkenyl;
—X₂—O—R₇; 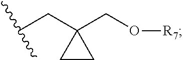
—X₂—NSO₂—R₇;
—C═C(R₉)—Y₁—O—R₇;
a C₃-C₆cycloalkyl;
a C₃-C₆heterocycloalkyl optionally substituted by a hydroxyl group;
a C₃-C₆cycloalkylene-Y₂—R₇;
a C₃-C₆heterocycloalkylene-Y₂—R₇ group, and
a heteroarylene-R₇ group optionally substituted by a linear or branched C₁-C₆alkyl group,
R₇ represents a group selected from the group consisting of: a linear or branched C₁-C₆alkyl group; a (C₃-C₆)cycloalkylene-R₈; 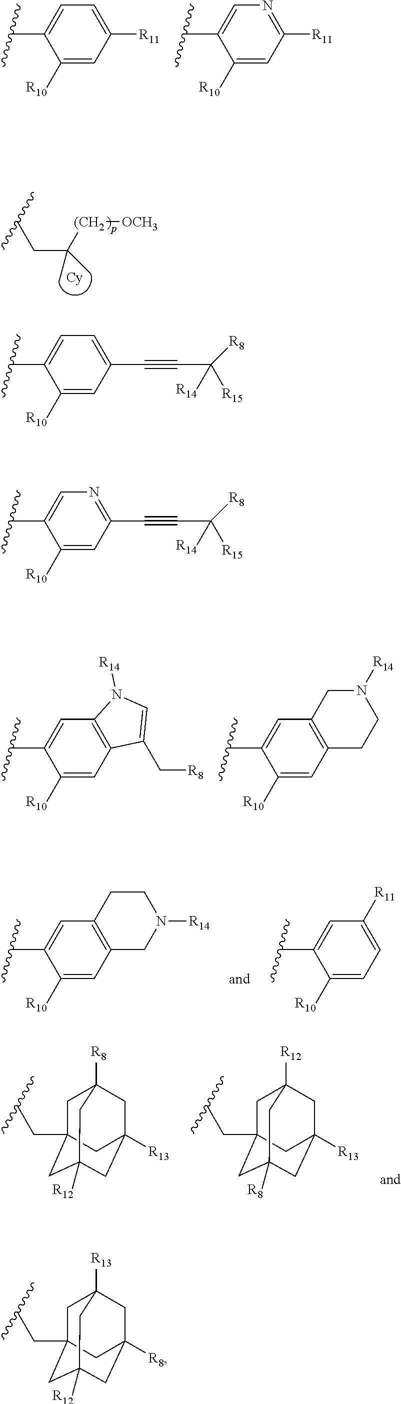
wherein Cy represents a C₃-C₈cycloalkyl,
R₈ represents a group selected from the group consisting of: hydrogen; linear or branched C₁-C₆alkyl, —NR′ₐR′_{b}; —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}; —X′₂—NR′ₐR′_{b}; —NR′_{c}—X′₂—N₃ and:
—NR′_{c}—X′₂-≡CH,
R₉ represents a group selected from the group consisting of a linear or branched C₁-C₆alkyl, trifluoromethyl, hydroxyl, a halogen, and a C₁-C₆alkoxy,
R₁₀ represents a group selected from the group consisting of hydrogen, fluorine, chlorine, bromine, —CF₃ and methyl,
R₁₁ represents a group selected from the group consisting of hydrogen, a C₁-C₃alkylene-R₈, a —O—C₁-C₃alkylene-R₈, —CO—NRₕRᵢ and a —CH═CH—C₁-C₄alkylene-NRₕRᵢ, —CH═CH—CHO, a C₃-C₈cycloalkylene-CH₂—R₈, and a C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ and X₂ independently of one another, represent a linear or branched C₁-C₆alkylene group optionally substituted by one or two groups selected from the group consisting of trifluoromethyl, hydroxyl, a halogen, and a C₁-C₆alkoxy,
X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O⁻; a C₁-C₆alkylene-COOH; a C₁-C₆alkylene-PO(OH)₂; a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy group; and the group: 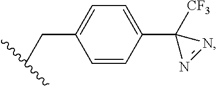 [SMILES: *Cc1ccc(C2(C(F)(F)F)N=N2)cc1]
or R′ₐ and R′_{b} form with the nitrogen atom carrying them a cycle B₃,
or R′ₐ, R′_{b} and R′_{c} form with the nitrogen atom carrying them a bridged C₃-C₈ hetero cycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈ heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, or —NH—SO₂—,
m=0, 1 or 2,
B₁, B₂, B₃ and B₄, independently of one another, represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl,
wherein one of the R₃ and R₈ groups, if present, is covalently attached to the linker, and wherein the valency of an atom is not exceeded by virtue of one or more substituents bonded thereto.

10. The compound of any one of claims 1-8, where D comprises a compound of Formula (I): 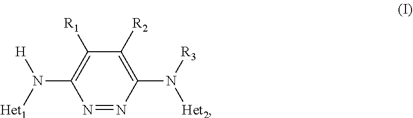
or an enantiomer, a diastereoisomer, and/or a pharmaceutically acceptable salt of any one of the foregoing, wherein:
R₁ and R₂ independently of one another represent a group selected from the group consisting of: hydrogen; a linear or branched C₁-C₆alkyl optionally substituted by a hydroxyl or a C₁-C₆alkoxy group; a C₃-C₆cycloalkyl; trifluoromethyl; and a linear or branched C₁-C₆alkylene-heterocycloalkyl wherein the heterocycloalkyl group is optionally substituted by a linear or branched C₁-C₆alkyl group;
or R₁ and R₂ form with the carbon atoms carrying them a C₃-C₆cycloalkylene group,
R₃ represents a group selected from the group consisting of: hydrogen; a C₃-C₆cycloalkyl; a linear or branched C₁-C₆alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:
—X₁-≡CH,
Rₐ and R_{b} independently of one another represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl groups; a C₁-C₆alkylene-SO₂OH; a C₁-C₆alkylene-SO₂O⁻; a C₁-C₆alkylene-COOH; a C₁-C₆alkylene-PO(OH)₂; a C₁-C₆alkylene-NR_{d}Rₑ; a C₁-C₆alkylene-N⁺R_{d}RₑR_{f}; a C₁-C₆alkylene-phenyl wherein the phenyl may be substituted by a C₁-C₆alkoxy group; and the group: 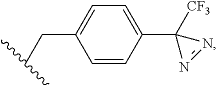 [SMILES: *Cc1ccc(C2(C(F)(F)F)N=N2)cc1]
or Rₐ and R_{b} form with the nitrogen atom carrying them a cycle B₁;
or Rₐ, R_{b} and R_{c} form with the nitrogen atom carrying them a bridged C₃-C₆hetero cycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a a cycle B₂,
or R_{d}, Rₑ and R_{f} form with the nitrogen atom carrying them a bridged C₃-C₆hetero cycloalkyl,
Het₁ represents a group selected from the group consisting of: 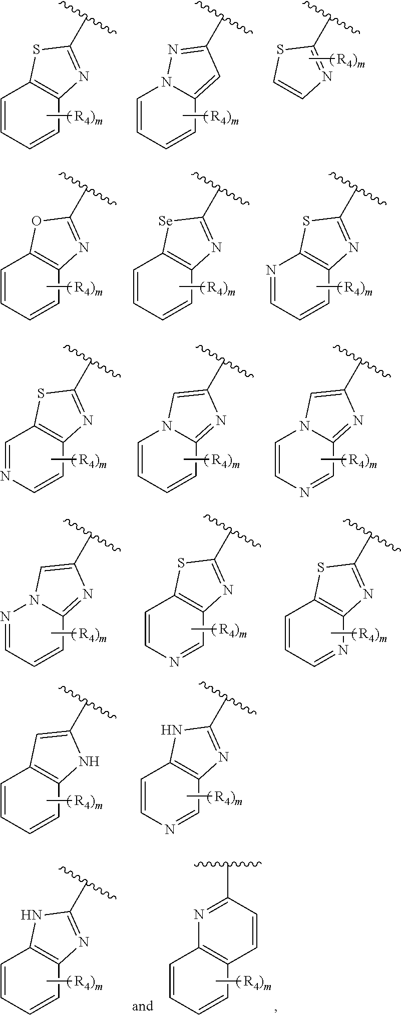
Het₂ represents a group selected from the group consisting of: 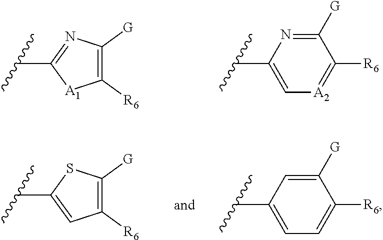
A₁ is —NH—, —N(C₁-C₃alkyl), O, S or Se,
A₂ is N, CH or C(R₅),
G is selected from the group consisting of:
—C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, a halogen, —NO₂, and —CN, in which:
R_{G1} and R_{G2} at each occurrence are each independently selected from the group consisting of hydrogen, a C₁-C₆alkyl optionally substituted by 1 to 3 halogen atoms, a C₂-C₆alkenyl, a C₂-C₆alkynyl, a C₃-C₆cycloalkyl, phenyl and —(CH₂)₁₋₄-phenyl;
R_{G3} is selected from the group consisting of a C₁-C₆alkyl optionally substituted by 1 to 3 halogen atoms, a C₂-C₆alkenyl, a C₂-C₆alkynyl, a C₃-C₆cycloalkyl, phenyl and —(CH₂)₁₋₄-phenyl; or R_{G1} and R_{G2}, together with the atom to which each is attached are combined to form a C₃-C₆heterocycloalkyl; or in the alternative, G is selected from the group consisting of: 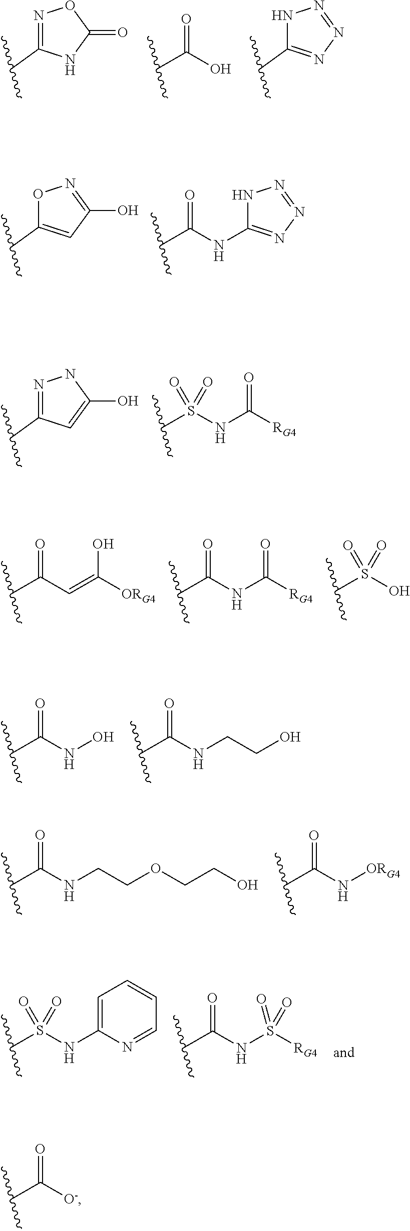
wherein R_{G4} is selected from the group consisting of a C₁-C₆alkyl optionally substituted by 1 to 3 halogen atoms, a C₂-C₆alkenyl, a C₂-C₆alkynyl and a C₃-C₆cycloalkyl,
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a C₂-C₆alkenyl; a C₂-C₆alkynyl; a halogen; and —CN,
Rₑ represents a group selected from the group consisting of:
hydrogen;
a linear or branched —C₁-C₆alkylene-R₈ group;
a —C₂-C₆alkenyl;
—X₂—O—R₇; 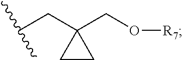
—X₂—NSO₂—R₇;
—C═C(R₉)—Y₁—O—R₇;
a C₃-C₆cycloalkyl;
a C₃-C₆heterocycloalkyl optionally substituted by a hydroxyl group;
a C₃-C₆cycloalkylene-Y₂—R₇;
a C₃-C₆heterocycloalkylene-Y₂—R₇ group, and
a heteroarylene-R₇ group optionally substituted by a linear or branched C₁-C₆alkyl group,
R₇ represents a group selected from the group consisting of: a linear or branched C₁-C₆alkyl group; a (C₃-C₆)cycloalkylene-R₈; 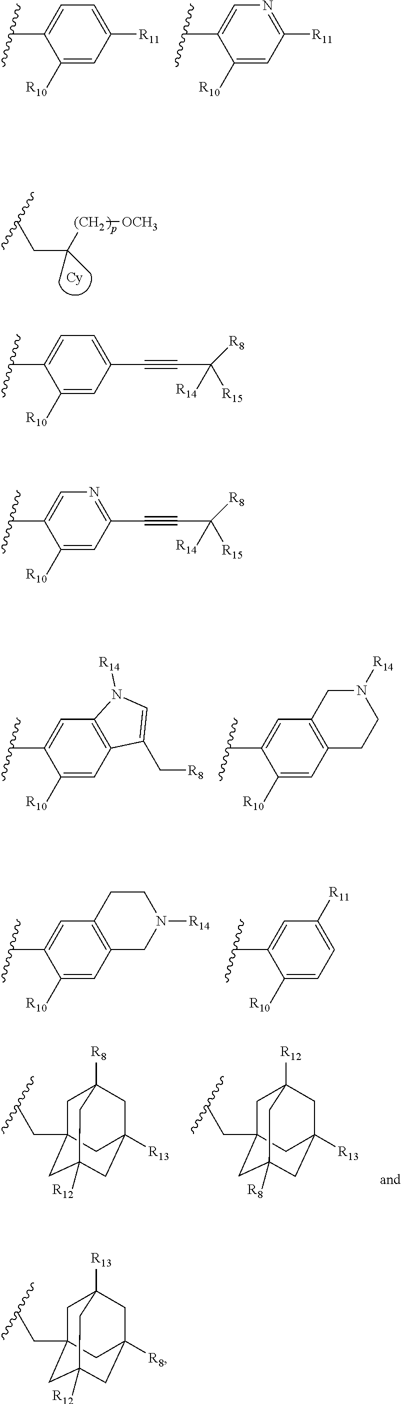
wherein Cy represents a C₃-C₈cycloalkyl,
R₈ represents a group selected from the group consisting of: hydrogen; linear or branched C₁-C₆ alkyl, —NR′ₐR′_{b}; —NRₐ—CO—OR_{c}; —NRₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}; —X′₂—NR′ₐR′_{b}; —NR′_{c}—X′₂—N₃ and:
—NR′_{c}—X′_{c}-≡CH,
R₉ represents a group selected from the group consisting of a linear or branched C₁-C₆ alkyl, trifluoromethyl, hydroxyl, a halogen, and a C₁-C₆ alkoxy,
R₁₀ represents a group selected from the group consisting of hydrogen, fluorine, chlorine, bromine, —CF₃ and methyl,
R₁₁ represents a group selected from the group consisting of hydrogen, a C₁-C₃alkylene-R₈, a —O—C₁-C₆alkylene-R₈, —CO—NRₕRᵢ and a —CH═CH—C₁-C₄alkylene-NRₕRᵢ, —CH═CH—CHO, a C₃-C₈cycloalkylene-CH₂—R₈, and a C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ and X₂ independently of one another, represent a linear or branched C₁-C₆alkylene group optionally substituted by one or two groups selected from the group consisting of trifluoromethyl, hydroxyl, a halogen, and a C₁-C₆alkoxy,
X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O⁻; a C₁-C₆alkylene-COOH; a C₁-C₆alkylene-PO(OH)₂; a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy group; and the group: 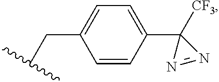 [SMILES: *Cc1ccc(C2(C(F)(F)F)N=N2)cc1]
or R′ₐ and R′_{b} form with the nitrogen atom carrying them a cycle B₃,
or R′ₐ, R′_{b} and R′_{c} form with the nitrogen atom carrying them a bridged C₃-C₈ hetero cycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈ heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, or —NH—SO₂—,
m=0, 1 or 2,
B₁, B₂, B₃ and B₄, independently of one another, represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl,
wherein one of the R₃ and R₈ groups, if present, is covalently attached to the linker, and wherein the valency of an atom is not exceeded by virtue of one or more substituents bonded thereto.

11. The compound of claim 9 or 10, wherein R, is a linear or branched C₁-C₆alkyl and R₂ is H.

12. The compound of any one of claims 1-8, where D comprises a compound of Formula (II): 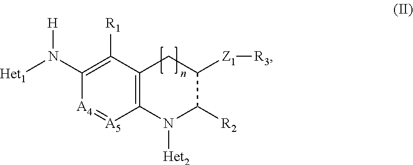
or an enantiomer, a diastereoisomer, and/or a pharmaceutically acceptable salt of any one of the foregoing, wherein:
n=0, 1 or 2,
------ represents a single or a double bond,
A₄ and A₅ independently of one another represent a carbon or a nitrogen atom,
Z₁ represents a bond, —N(R)—, or —O—, wherein R represents a hydrogen or a linear or branched C₁-C₆alkyl,
R₁ represents a group selected from the group consisting of: hydrogen; a linear or branched C₁-C₆alkyl optionally substituted by a hydroxyl or a C₁-C₆alkoxy group; a C₃-C₆cycloalkyl; trifluoromethyl; and a linear or branched C₁-C₆alkylene-heterocycloalkyl wherein the heterocycloalkyl group is optionally substituted by a linear or branched C₁-C₆alkyl group;
R₂ represents a hydrogen or a methyl;
R₃ represents a group selected from the group consisting of: hydrogen; a linear or branched C₁-C₄alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:
—X₁-≡CH,
Rₐ and R_{b} independently of one another represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl groups; a C₁-C₆alkylene-SO₂OH; a C₁-C₆alkylene-SO₂O⁻; a C₁-C₆alkylene-COOH; a C₁-C₆alkylene-PO(OH)₂; a C₁-C₆alkylene-NR_{d}Rₑ; a C₁-C₆alkylene-N⁺R_{d}RₑR_{f}; a C₁-C₆alkylene-phenyl wherein the phenyl may be substituted by a C₁-C₆alkoxy group; and the group: 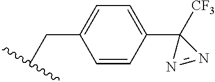 [SMILES: *Cc1ccc(C2(C(F)(F)F)N=N2)cc1]
or Rₐ and R_{b} form with the nitrogen atom carrying them a cycle B₁;
or Rₐ, R_{b} and R_{c} form with the nitrogen atom carrying them a bridged C₃-C₈ heterocycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a cycle B₂,
or R_{d}, Rₑ and R_{f} form with the nitrogen atom carrying them a bridged C₃-C₈ heterocycloalkyl,
Het₁ represents a group selected from the group consisting of: 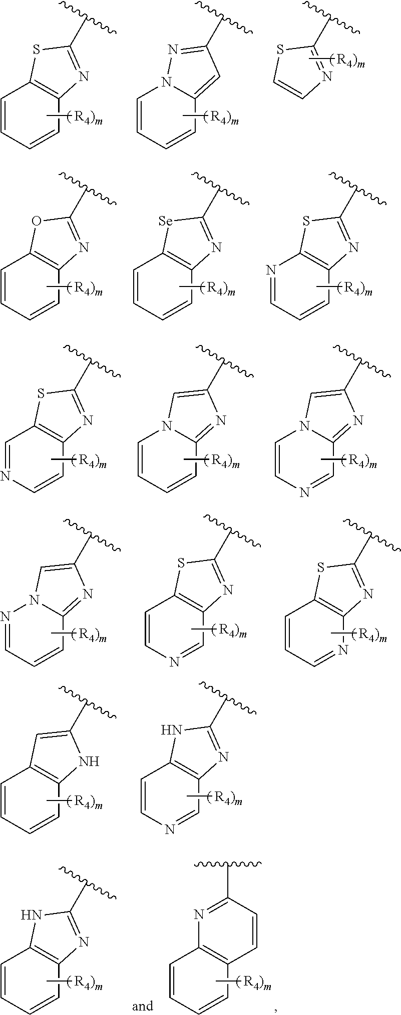
Het₂ represents a group selected from the group consisting of: 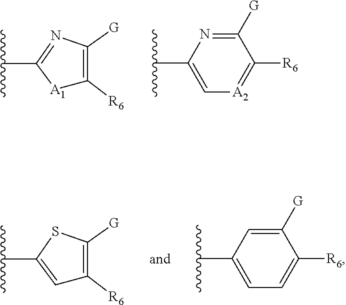
A₁, is —NH—, —N(C₁-C₆alkyl), O, S or Se,
A₂ is N, CH or C(R₅),
G is selected from the group consisting of:
—C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, —C₁-C₆alkyl optionally substituted by a hydroxyl group, a halogen, —NO₂, and —CN, in which:
R_{G1} and R_{G2} at each occurrence are each independently selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂-C₆ alkynyl, a C₃-C₆cycloalkyl, phenyl and —(CH₂)₁₋₄-phenyl;
R_{G3} is selected from the group consisting of a C₁-C₆alkyl optionally substituted by 1 to 3 halogen atoms, a C₂-C₆alkenyl, a C₂-C₆alkynyl, a C₃-C₆cycloalkyl, phenyl and —(CH₂)₁₋₄-phenyl; or R_{G1} and R_{G2}, together with the atom to which each is attached are combined to form a C₃-C₆heterocycloalkyl; or in the alternative, G is selected from the group consisting of: 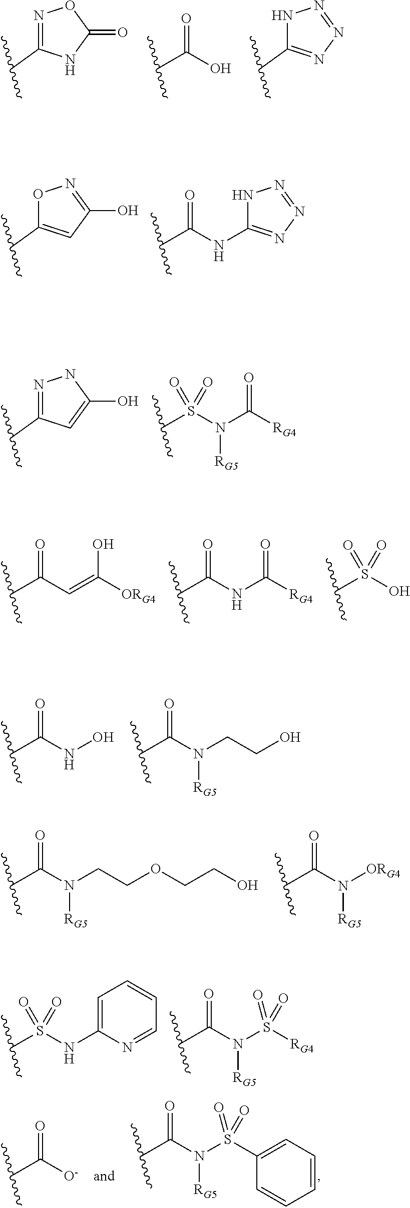
wherein R_{G4} is selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆ alkenyl, a C₂-C₆alkynyl and a C₃-C₆cycloalkyl, and R_{G5} represents a hydrogen atom or a C₁-C₆alkyl group optionally substituted by 1 to 3 halogen atoms,
R⁴ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a C₂-C₆alkenyl; a C₂-C₆alkynyl; a halogen and —CN,
R₆ represents a group selected from the group consisting of:
hydrogen;
a linear or branched —C₁-C₆alkylene-R₈ group;
a —C₂-C₆alkenyl;
—X₂—O—R₇; 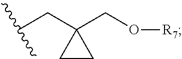
—X₂—NSO₂—R₇;
—C═C(R₉)—Y₁—O—R₇;
a C₃-C₆cycloalkyl;
a C₃-C₆heterocycloalkyl optionally substituted by a hydroxyl group;
a C₃-C₆cycloalkylene-Y₂—R₇;
a C₃-C₆heterocycloalkylene-Y₂—R₇ group, and
a heteroarylene-R₇ group optionally substituted by a linear or branched C₁-C₆alkyl group,
R₇ represents a group selected from the group consisting of: a linear or branched C₁-C₆alkyl group; a (C₃-C₆)cycloalkylene-R₈; 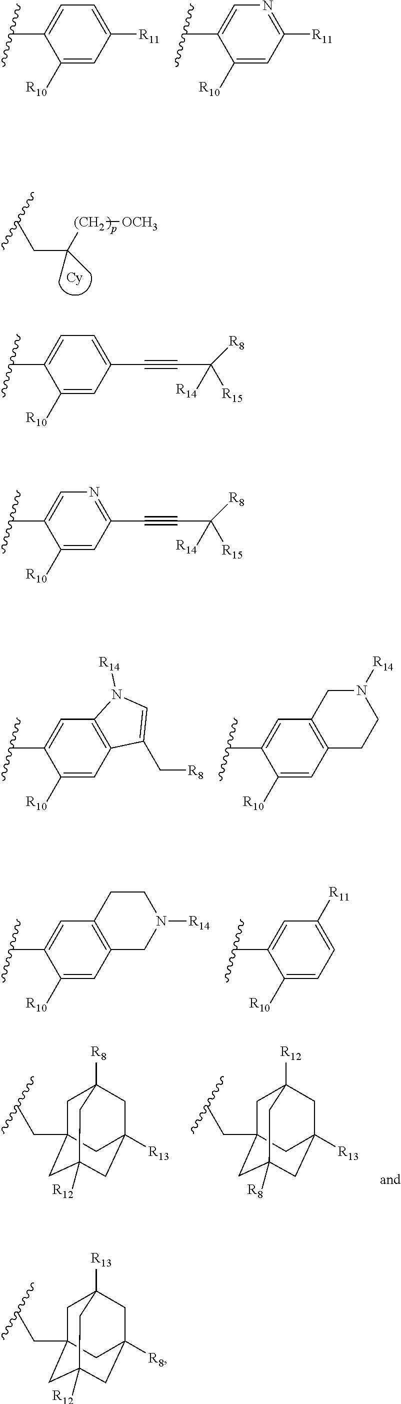
wherein Cy represents a C₃-C₆cycloalkyl,
R₈ represents a group selected from the group consisting of: hydrogen; a linear or branched C₁-C₆alkyl, —NR′ₐR′_{b}; —NR′ₐ—CO—OR′_{c}; —NR′ₐ—CO—R′_{c}; —N⁺R′_{c}R′_{b}R′_{c}; —O—R′_{c}; —NH—X′₂—N⁺R′ₐR′_{b}R′_{c}; —O—X′₂—NR′ₐR′_{b}, —X′₂—NR′ₐR′_{b}, —NR′_{c}—X′₂—N₃ and
—NR′_{c}—X′₂-≡CH,
R₉ represents a group selected from the group consisting of a linear or branched C₁-C₆alkyl, trifluoromethyl, hydroxyl, a halogen, and a C₁-C₆alkoxy,
R₁₀ represents a group selected from the group consisting of hydrogen, fluorine, chlorine, bromine, —CF₃ and methyl,
R₁₁ represents a group selected from the group consisting of hydrogen, a halogen, a C₁-C₃alkylene-R₈, a —O—C₁-C₆alkylene-R₈, —CO—NRₕRᵢ and a —CH═CH—C₁-C₄alkylene-NRₕRᵢ, —CH═CH—CHO, a C₃-C₈cycloalkylene-CH₂—R₈, and a C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ represents a linear or branched C₁-C₄alkylene group optionally substituted by one or two groups selected from the group consisting of trifluoromethyl, hydroxyl, a halogen, and a C₁-C₆alkoxy,
X₂ represents a linear or branched C₁-C₆alkylene group optionally substituted by one or two groups selected from the group consisting of trifluoromethyl, hydroxyl, a halogen, and a C₁-C₆alkoxy,
X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O⁻; a C₁-C₆alkylene-COOH; a C₁-C₆alkylene-PO(OH)₂; a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy group; and the group: 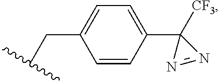 [SMILES: *Cc1ccc(C2(C(F)(F)F)N=N2)cc1]
or R′ₐ and R′_{b} form with the nitrogen atom carrying them a cycle B₃,
or R′ₐ, R′_{b} and R′_{c} form with the nitrogen atom carrying them a bridged C₃-C₈ heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈ heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, or —NH—SO₂—,
m=0, 1 or 2,
B₁, B₂, B₃ and B₄, independently of one another, represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl,
wherein one of the R₃, R₈ and G groups, if present, is covalently attached to the linker, and wherein the valency of an atom is not exceeded by virtue of one or more substituents bonded thereto.

13. The compound of any one of claims 1-8, where D comprises a compound of Formula (II): 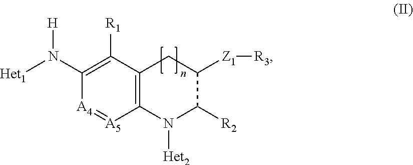
or an enantiomer, a diastereoisomer, and/or a pharmaceutically acceptable salt of any one of the foregoing, wherein:
n=0, 1 or 2,
------ represents a single or a double bond,
A₄ and A₅ independently of one another represent a carbon or a nitrogen atom,
Z₁ represents a bond, —N(R)—, or —O—, wherein R represents a hydrogen or a linear or branched C₁-C₆alkyl,
R₁ represents a group selected from the group consisting of: hydrogen; a linear or branched C₁-C₆alkyl optionally substituted by a hydroxyl or a C₁-C₆alkoxy group; a C₃-C₆cycloalkyl; trifluoromethyl; and a linear or branched C₁-C₆alkylene-heterocycloalkyl wherein the heterocycloalkyl group is optionally substituted by a linear or branched C₁-C₆alkyl group;
R₂ represents a hydrogen or a methyl;
R₃ represents a group selected from the group consisting of: hydrogen; a linear or branched C₁-C₄alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:
—X₁-≡CH,
Rₐ and R_{b} independently of one another represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl groups; a C₁-C₆alkylene-SO₂OH; a C₁-C₆alkylene-SO₂O⁻; a C₁-C₆alkylene-COOH; a C₁-C₆alkylene-PO(OH)₂; a C₁-C₆alkylene-NR_{d}Rₑ; a C₁-C₆alkylene-N⁺R_{d}RₑR_{f}; a C₁-C₆alkylene-phenyl wherein the phenyl may be substituted by a C₁-C₆alkoxy group; and the group: 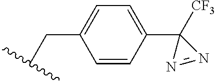 [SMILES: *Cc1ccc(C2(C(F)(F)F)N=N2)cc1]
or Rₐ and R_{b} form with the nitrogen atom carrying them a cycle B₁;
or Rₐ, R_{b} and R_{c} form with the nitrogen atom carrying them a bridged C₃-C₈ heterocycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a cycle B₂,
or R_{d}, Rₑ and R_{f} form with the nitrogen atom carrying them a bridged C₃-C₈ heterocycloalkyl,
Het₁ represents a group selected from the group consisting of: 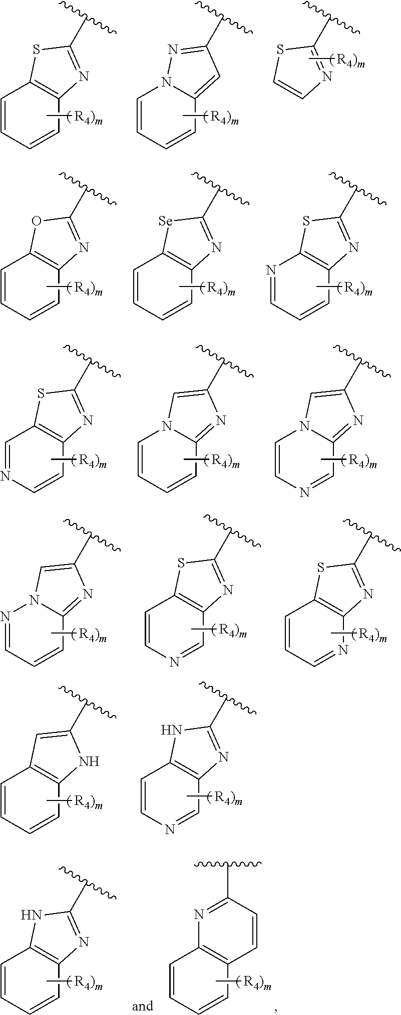
Het₂ represents a group selected from the group consisting of: 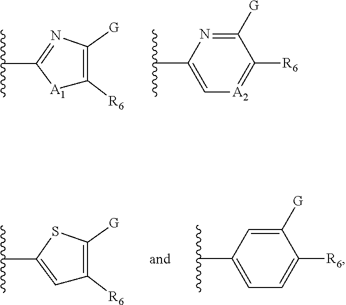
A₁ is —NH—, —N(C₁-C₃alkyl), O, S or Se,
A₂ is N, OH or C(R₅),
G is selected from the group consisting of:
—C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, a halogen, —NO₂, and —CN, in which:
R_{G1} and R_{G2} at each occurrence are each independently selected from the group consisting of hydrogen, a CO—C₆alkyl optionally substituted by 1 to 3 halogen atoms, a C₂-C₆alkenyl, a C₂-C₆ alkynyl, a C₃-C₆cycloalkyl, phenyl and —(CH₂)₁₋₄-phenyl;
R_{G3} is selected from the group consisting of a CO-C₆alkyl optionally substituted by 1 to 3 halogen atoms, a C₂-C₆alkenyl, a C₂-C₆alkynyl, a C₃-C₆cycloalkyl, phenyl and —(CH₂)₁₋₄-phenyl; or R_{G1} and R_{G2}, together with the atom to which each is attached are combined to form a C₃-C₈heterocycloalkyl; or in the alternative, G is selected from the group consisting of: 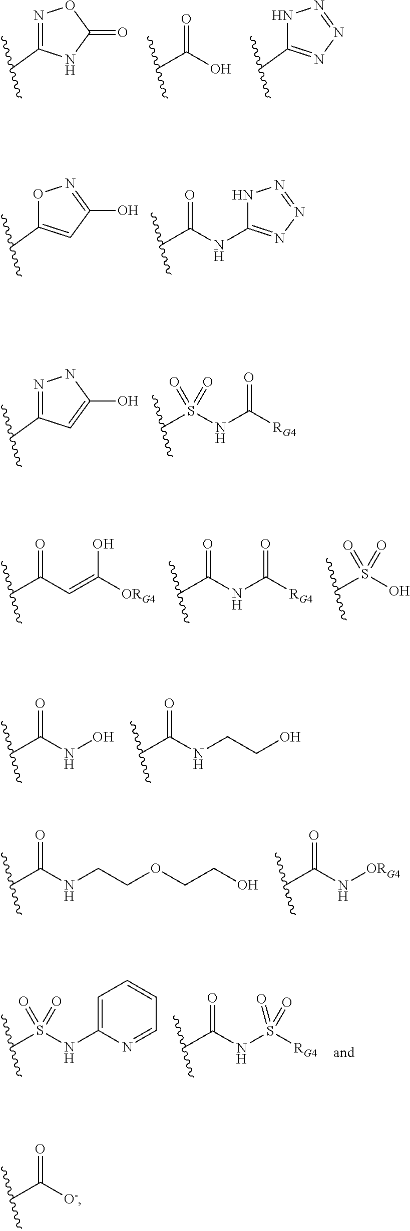
wherein R_{G4} is selected from the group consisting of a C₁-C₆alkyl optionally substituted by 1 to 3 halogen atoms, a C₂-C₆ alkenyl, a C₂-C₆alkynyl and a C₃-C₆cycloalkyl,
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a C₂-C₆alkenyl; a C₂-C₆alkynyl; a halogen and —CN,
Rₑ represents a group selected from the group consisting of:
hydrogen;
a linear or branched —C₁-C₆alkylene-R₈ group;
a —C₂-C₆alkenyl;
—X₂—O—R₇; 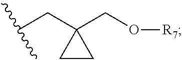
—X₂—NSO₂—R₇;
—C═C(R₉)—Y₁—O—R₇;
a C₃-C₆cycloalkyl;
a C₃-C₆heterocycloalkyl optionally substituted by a hydroxyl group;
a C₃-C₆cycloalkylene-Y₂—R₇;
a C₃-C₆heterocycloalkylene-Y₂—R₇ group, and
a heteroarylene-R₇ group optionally substituted by a linear or branched C₁-C₆alkyl group,
R₇ represents a group selected from the group consisting of: a linear or branched C₁-C₆alkyl group; a (C₃-C₆)cycloalkylene-R₈; 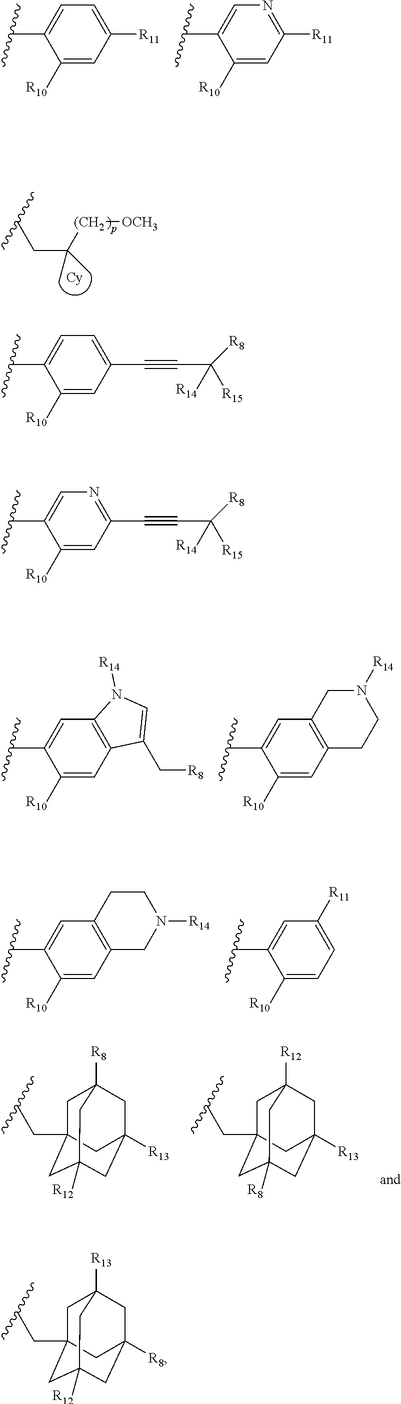
wherein Cy represents a C₃-C₈cycloalkyl,
R₈ represents a group selected from the group consisting of: hydrogen; a linear or branched C₁-C₆alkyl, —NR′ₐR′_{b}; —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}, —X′₂—NR′ₐR′_{b}, —NR′_{c}—X′₂—N₃ and:
—NR′_{c}—X′₂-≡CH,
R₉ represents a group selected from the group consisting of a linear or branched C₁-C₆alkyl, trifluoromethyl, hydroxyl, a halogen, and a C₁-C₆alkoxy,
R₁₀ represents a group selected from the group consisting of hydrogen, fluorine, chlorine, bromine, —CF₃ and methyl,
R₁₁ represents a group selected from the group consisting of hydrogen, a halogen, a C₁-C₃alkylene-R₈, a —O—C₁-C₃alkylene-R₈, —CO—NRₕRᵢ and a —CH═CH—C₁-C₄alkylene-NRₕRᵢ, —CH═CH—CHO, a C₃-C₈cycloalkylene-CH₂—R₈, and a C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ represents a linear or branched C₁-C₄alkylene group optionally substituted by one or two groups selected from the group consisting of trifluoromethyl, hydroxyl, a halogen, and a C₁-C₆alkoxy,
X₂ represents a linear or branched C₁-C₆alkylene group optionally substituted by one or two groups selected from the group consisting of trifluoromethyl, hydroxyl, a halogen, and a C₁-C₆alkoxy,
X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O⁻; a C₁-C₆alkylene-COOH; a C₁-C₆alkylene-PO(OH)₂; a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy group; and the group: 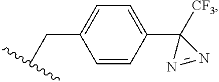 [SMILES: *Cc1ccc(C2(C(F)(F)F)N=N2)cc1]
or R′ₐ and R′_{b} form with the nitrogen atom carrying them a cycle B₃,
or R′ₐ, R′_{b} and R′_{c} form with the nitrogen atom carrying them a bridged C₃-C₈ heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈ heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, or —NH—SO₂—,
m=0, 1 or 2,
B₁, B₂, B₃ and B₄, independently of one another, represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched CO-C₆alkyl, hydroxyl, —NH₂, oxo and piperidinyl,
wherein one of the R₃ and R₈ groups, if present, is covalently attached to the linker, and wherein the valency of an atom is not exceeded by virtue of one or more substituents bonded thereto.

14. The compound of claim 12 or 13, wherein A₄ and A₅ both represent a nitrogen atom, R₁ is a linear or branched C₁₋₆alkyl; R₂ is H; n is 1; and ----- represents a single bond.

15. The compound of any one of claims 1-14, wherein D comprises a compound of formula (IA) or (IIA): 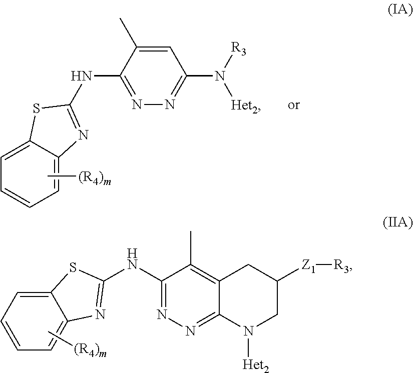
or an enantiomer, a diastereoisomer, and/or a pharmaceutically acceptable salt of any one of the foregoing, wherein:
Z₁ represents a bond or —O—,
R₃ represents a group selected from the group consisting of: hydrogen; a C₃-C₆cycloalkyl; a linear or branched C₁-C₆alkyl; —X₁—NRₐR_{b}; —X₁—N⁺RₐR_{b}R_{c}; —X₁—O—R_{c}; —X₁—N₃ and —X₁-≡CH,
Rₐ and R_{b} independently of one another represent a group selected from the group consisting of: hydrogen; a linear or branched C₁-C₆alkyl optionally substituted by one or two hydroxyl groups; and a C₁-C₆alkylene-SO₂O⁻,
R_{c} represents a hydrogen or a linear or branched C₁-C₆alkyl group,
Het₂ represents a group selected from the group consisting of: 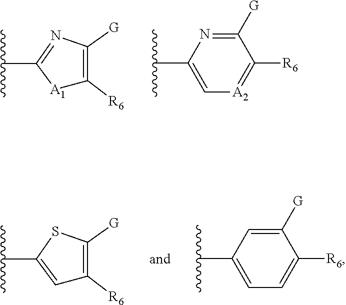
A₁ is —NH—, —N(C₁-C₆alkyl), O, S or Se,
A₂ is N, CH or C(R₅),
G is selected from the group consisting of:
—C(O)OH, —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, —C₁-C₆alkyl optionally substituted by a hydroxyl group, —C(O)NR_{G5}S(O)₂R_{G4}, halogen, —NO₂, and —CN, in which:
R_{G}, R_{G2}, R_{G4} and R_{G5} at each occurrence are each independently selected from the group consisting of hydrogen, and a C₁-C₆alkyl optionally substituted by 1 to 3 halogen atoms;
R_{G3} is a C₁-C₆alkyl optionally substituted by 1 to 3 halogen atoms; or
R_{G1} and R_{G2}, together with the atom to which each is attached are combined to form a C₃-C₈heterocycloalkyl;
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a halogen and —CN,
R₆ represents a group selected from the group consisting of:
a linear or branched —C₁-C₆alkylene-R₈ group;
—X₂—O—R₇; and
a heteroarylene-R₇ group optionally substituted by a linear or branched C₁-C₆alkyl group,
R₇ represents a group selected from the group consisting of: a linear or branched C₁-C₆alkyl group; (C₃-C₆)cycloalkylene-R₈; 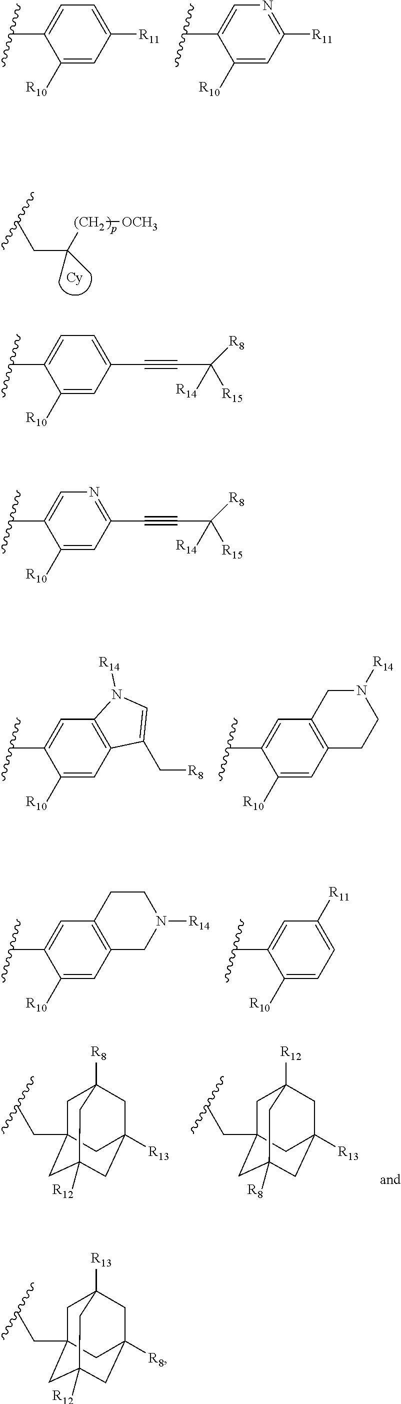
wherein Cy represents a C₃-C₈cycloalkyl,
R₈ represents a group selected from the group consisting of: hydrogen; a linear or branched C₁-C₆alkyl, —NR′ₐR′_{b}; NR′ₐ—CO—OR′_{c} NR′ₐCO—R′_{c}; —N⁺R′ₐR′_{b}R′_{c}—O—R′_{c}; —NH—X′₂—N⁺R′ₐR′_{b}R′_{c}; —O—X′₂—NR′ₐR′_{b}; —X′₂—NR′ₐR′_{b}; —NR′_{c}—X′₂—N₃ and:
—NR′_{c}—X′₂-≡CH,
R₁₀ represents a group selected from the group consisting of hydrogen, fluorine, chlorine, bromine, —CF₃ and methyl,
R₁₁ represents a group selected from the group consisting of hydrogen, a C₁-C₃alkylene-R₈, —O—C₁-C₆alkylene-R₈, —CO—NRₕRᵢ, —CH═CH—C₁-C₄alkylene-NRₕRᵢ, —CH═CH—CHO, a C₃-C₈cycloalkylene-CH₂—R₈, and a C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group,
or R₁₄ and R₁₅ form with the carbon atom carrying them a
a cyclohexyl,
Rₕ and Rᵢ, independently of one another, represent a hydrogen or a linear or branched C₁-C₆alkyl group,
X₁ and X₂ independently of one another, represent a linear or branched C₁-C₆alkylene group optionally substituted by one or two groups selected from the group consisting of trifluoromethyl, hydroxyl, a halogen, and C₁-C₆alkoxy,
X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O⁻; a C₁-C₆alkylene-COOH; a C₁-C₆alkylene-PO(OH)₂; a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy group; and the group: 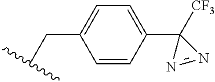 [SMILES: *Cc1ccc(C2(C(F)(F)F)N=N2)cc1]
or R′ₐ and R′_{b} form with the nitrogen atom carrying them a cycle B₃,
or R′ₐ, R′_{b} and R′_{c} form with the nitrogen atom carrying them a bridged C₃-C₈heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₆heterocycloalkyl,
m=0, 1 or 2,
p=1, 2, 3 or 4,
B₃ and B₄, independently of one another, represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl.

16. The compound of any one of claims 1-14, wherein D comprises a compound of formula (IA) or (IIA): 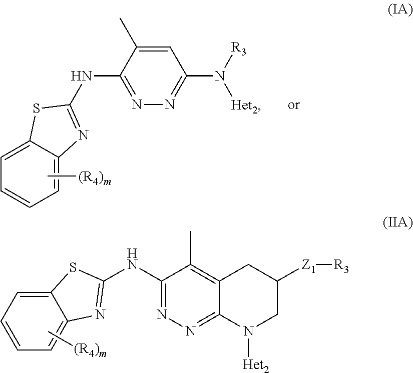
or an enantiomer, a diastereoisomer, and/or a pharmaceutically acceptable salt of any one of the foregoing, wherein:
Z₁ represents a bond or —O—,
R₃ represents a group selected from the group consisting of: hydrogen; a C₃-C₆cycloalkyl; a linear or branched C₁-C₆alkyl; —X₁—NRₐR_{b}; —X₁—N⁺RₐR_{b}R_{c}; —X₁—O—R_{c}; —X₁—N₃ and —X₁-≡CH,
Rₐ and R_{b} independently of one another represent a group selected from the group consisting of: hydrogen; a linear or branched C₁-C₆alkyl optionally substituted by one or two hydroxyl groups; and a C₁-C₆alkylene-SO₂O⁻,
R_{c} represents a hydrogen or a linear or branched C₁-C₆alkyl group,
Het₂ represents a group selected from the group consisting of: 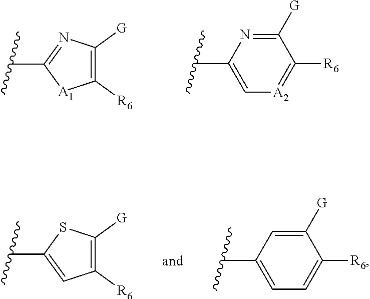
A₁ is —NH—, —N(C₁-C₆alkyl), O, S or Se,
A₂ is N, CH or C(R₅),
G is selected from the group consisting of:
—C(O)OH, —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, halogen, —NO₂, and —CN, in which:
R_{G1} and R_{G2} at each occurrence are each independently selected from the group consisting of hydrogen, and a C₁-C₆alkyl optionally substituted by 1 to 3 halogen atoms;
R_{G3} is a C₁-C₆alkyl optionally substituted by 1 to 3 halogen atoms; or
R_{G1} and R_{G2}, together with the atom to which each is attached are combined to form a C₃-C₈heterocycloalkyl;
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a halogen and —CN,
R₆ represents a group selected from the group consisting of:
a linear or branched —C₁-C₆alkylene-R₈ group;
—X₂—O—R₇; and
a heteroarylene-R₇ group optionally substituted by a linear or branched C₁-C₆alkyl group,
R₇ represents a group selected from the group consisting of: a linear or branched C₁-C₆alkyl group; (C₃-C₆)cycloalkylene-R₆; 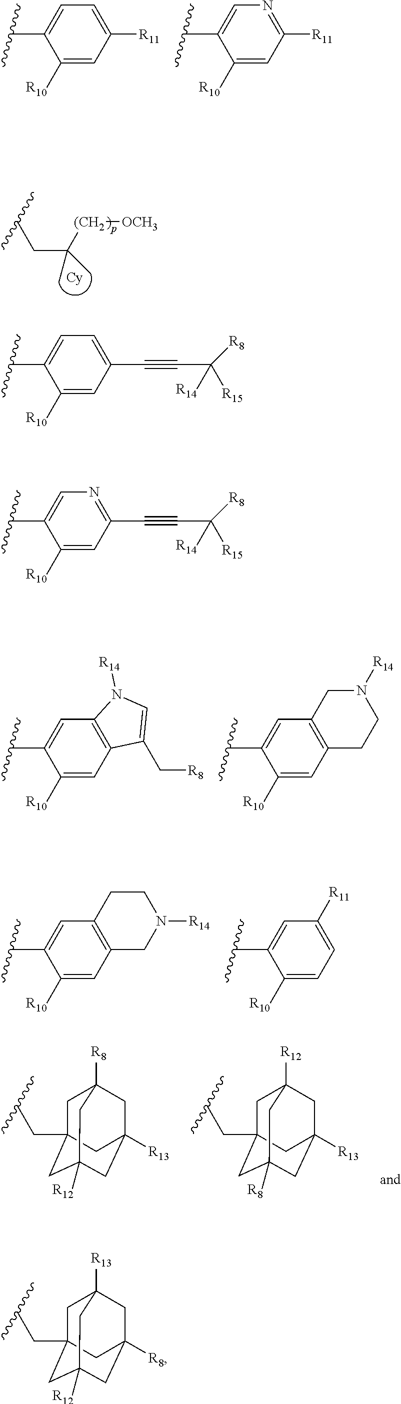
wherein Cy represents a C₃-C₆cycloalkyl,
R₈ represents a group selected from the group consisting of: hydrogen; a linear or branched C₁-C₆alkyl, —NR′ₐR′_{b}; —NR′ₐ—CO—OR′_{c}; —NR′ₐ—CO—R′_{c}; —N⁺R′_{c}R′_{b}R′_{c}; —O—R′_{c}; —NH—X′₂—N⁺R′ₐR′_{b}R′_{c}; —O—X′₂—NR′ₐR′_{b}, —X′₂—NR′ₐR′_{b}, —NR′_{c}—X′₂—N₃ and
—NR′_{c}—X′₂-≡CH,
R₁₀ represents a group selected from the group consisting of hydrogen, fluorine, chlorine, bromine, —CF₃ and methyl,
R₁₁ represents a group selected from the group consisting of hydrogen, a halogen, a C₁-C₃alkylene-R₈, a —O—C₁-C₆alkylene-R₈, —CO—NRₕRᵢ and a —CH═CH—C₁-C₄alkylene-NRₕRᵢ, —CH═CH—CHO, a C₃-C₈cycloalkylene-CH₂—R₈, and a C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a a cyclohexyl,
Rₕ and Rᵢ, independently of one another, represent a hydrogen or a linear or branched C₁-C₆alkyl group,
X₁ and X₂ independently of one another, represent a linear or branched C₁-C₆alkylene group optionally substituted by one or two groups selected from the group consisting of trifluoromethyl, hydroxyl, a halogen, and C₁-C₆alkoxy,
X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O⁻; a C₁-C₆alkylene-COOH; a C₁-C₆alkylene-PO(OH)₂; a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy group; and the group: 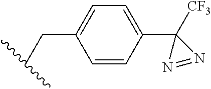 [SMILES: *Cc1ccc(C2(C(F)(F)F)N=N2)cc1]
or R′ₐ and R′_{b} form with the nitrogen atom carrying them a cycle B₃,
or R′ₐ, R′_{b} and R′_{c} form with the nitrogen atom carrying them a bridged C₃-C₆heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₆heterocycloalkyl,
m=0, 1 or 2,
p=1, 2, 3 or 4,
B₃ and B₄, independently of one another, represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl.

17. The compound of any one of claims 1-16, wherein R, represents a group selected from the group consisting of: a linear or branched C₁-C₆alkyl group; a (C₃-C₆)cycloalkylene-R₈; 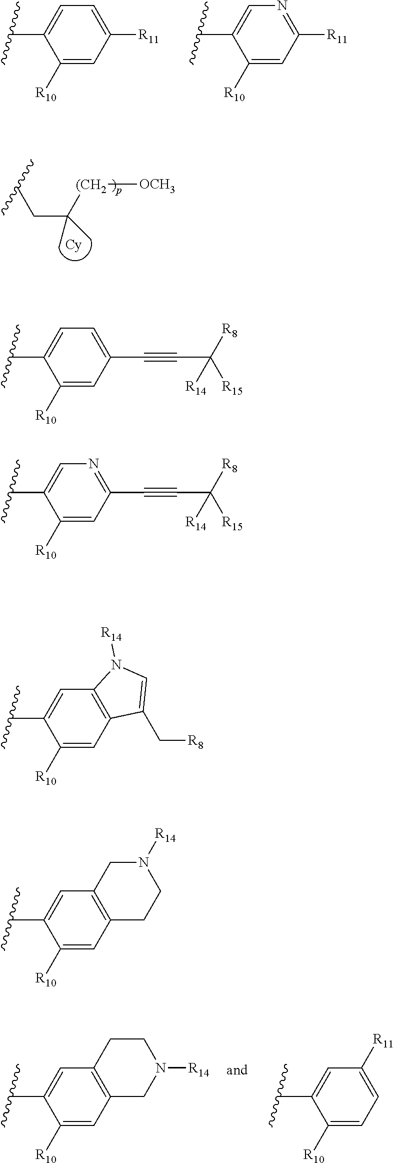
wherein Cy represents a C₃-C₈cycloalkyl.

18. The compound of any one of claims 1-17, wherein R, represents a group selected from the group consisting of:

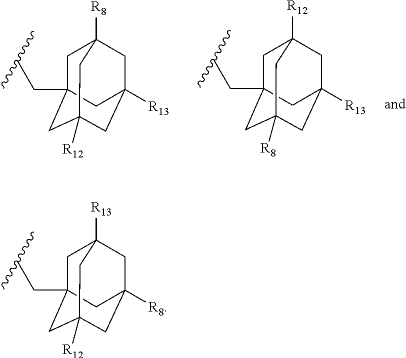

19. The compound of any one of claims 1-18, wherein D comprises a compound of formula (IB), (IC-1), (IIB) or (IIC-1): 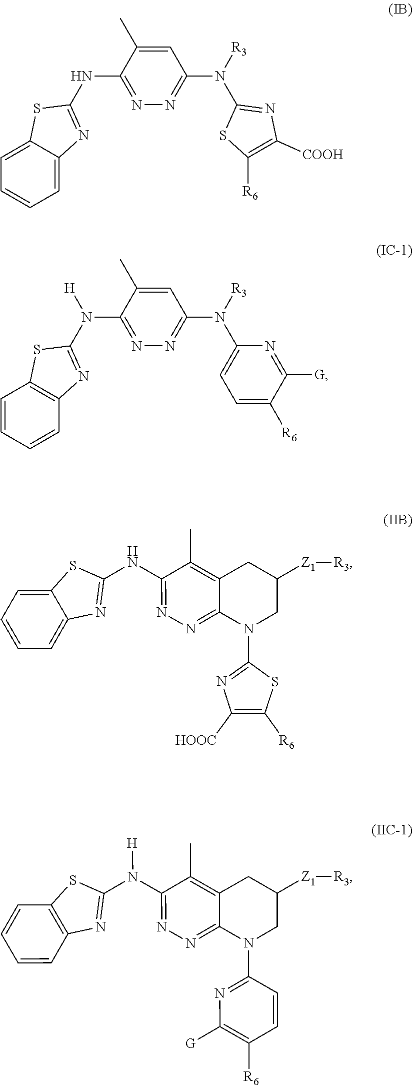
or an enantiomer, a diastereoisomer, and/or a pharmaceutically acceptable salt of any one of the foregoing, wherein:
for formula (IB) or (IC-1), R₃ represents a group selected from the group consisting of: hydrogen; a linear or branched C₁-C₆alkyl; —X₁—NRₐR_{b}; —X₁—N⁺RₐR_{b}R_{c}; —X₁—O—R_{c}; —X₁—N₃ and —X₁-≡CH,
for formula (IIB) or (IIC-1), Z₁ represents a bond, and R₃ represents hydrogen; or Z₁ represents —O—, and R₃ represents —X₁—NRₐR_{b},
for formula (IC-1), G is selected from the group consisting of —C(O)OH and —C(O)NR_{G1}R_{G2};
for formula (IIC-1), G is selected from the group consisting of —C(O)OH, —C(O)NR_{G1}R_{G2}, —C(O)R_{G2}, —C₁-C₆alkyl optionally substituted by a hydroxyl group and —C(O)NR_{G5}S(O)₂R_{G4}, in which R_{G1}, R_{G2}, R_{G4} and R_{G5} at each occurrence are each independently selected from the group consisting of hydrogen and a C₁-C₆alkyl optionally substituted by 1 to 3 halogen atoms;
Rₐ and R_{b} independently of one another represent a group selected from the group consisting of: hydrogen; a linear or branched C₁-C₆alkyl optionally substituted by one or two hydroxyl groups; and a C₁-C₆alkylene-SO₂O⁻,
R_{c} represents a hydrogen or a linear or branched C₁-C₆alkyl group
R₆ represents a linear or branched —C₁-C₆alkylene-R₈ group, —X₂—O—R₇, or a heteroarylene-R₇ group optionally substituted by a linear or branched C₁-C₆alkyl group,
R₇ represents a group selected from the group consisting of: 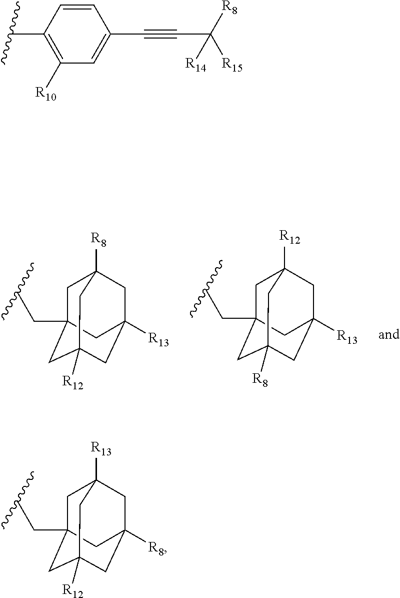
R₈ represents a group selected from the group consisting of: —NR′ₐR′_{b}; —O—X′₂—NR′ₐR′_{b};
and —X′₂—NR′ₐR′_{b},
R₁₀ represents fluorine,
R₁₂ and R₁₃, independently of one another, represent a hydrogen atom or a methyl group,
R₁₄ and R₁₅, independently of one another, represent a hydrogen or a methyl group,
X₁ and X₂ independently of one another, represent a linear or branched C₁-C₆alkylene group optionally substituted by one or two groups selected from the group consisting of trifluoromethyl, hydroxyl, a halogen, and a C₁-C₆alkoxy,
X′₂ represents a linear or branched C₁-C₆alkylene,
R′ₐ and R′_{b} independently of one another, represent a group selected from the group consisting of: hydrogen; a linear or branched C₁-C₆alkyl optionally substituted by one or two hydroxyl or C₁-C₆alkoxy groups; and a C₁-C₆alkylene-NR′_{d}R′ₑ;
or R′ₐ and R′_{b} form with the nitrogen atom carrying them a cycle B₃,
R′_{d}, R′ₑ independently of one another, represents a hydrogen or a linear or branched C₁-C₆alkyl group,
B₃ represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen and nitrogen, (iii) be substituted by one or two groups selected from the group consisting of: fluorine, bromine, chlorine, a linear or branched C₁-C₆alkyl, hydroxyl, and oxo.

20. The compound of any one of claims 1-18, wherein D comprises a compound of formula (IB), (IC), (IIB) or (IIC): 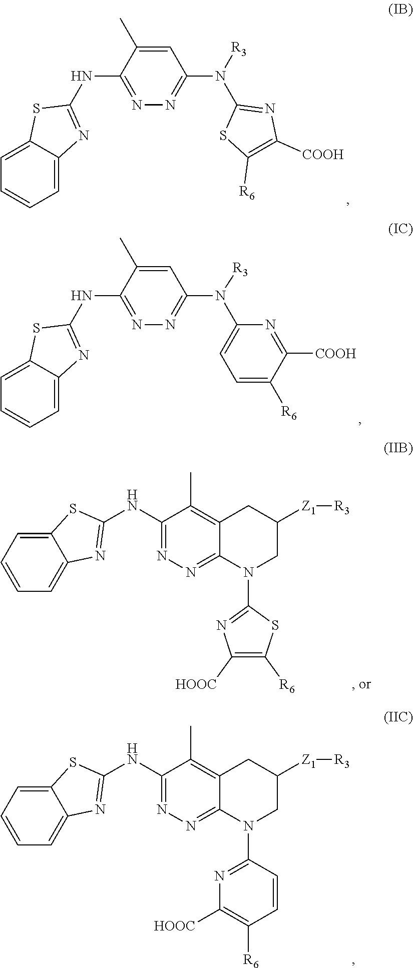
or an enantiomer, a diastereoisomer, and/or a pharmaceutically acceptable salt of any one of the foregoing, wherein:
for formula (IB) or (IC), R₃ represents a group selected from the group consisting of:
hydrogen; a linear or branched C₁-C₆alkyl; —X₁—NRₐR_{b}; —X₁—N⁺RₐR_{b}R_{c}; —X₁—O—R_{c}; —X₁—N₃ and —X₁-≡CH, for formula (IIB) or (IIC), Z₁ represents a bond, and R₃ represents hydrogen; or Z₁ represents —O—, and R₃ represents —X₁—NRₐR_{b},
Rₐ and R_{b} independently of one another represent a group selected from the group consisting of: hydrogen; a linear or branched C₁-C₆alkyl optionally substituted by one or two hydroxyl groups; and a C₁-C₆alkylene-SO₂O⁻,
R_{c} represents a hydrogen or a linear or branched C₁-C₆alkyl group
R₆ represents a linear or branched —C₁-C₆alkylene-R₈ group, —X₂—O—R₇, or a heteroarylene-R₇ group optionally substituted by a linear or branched C₁-C₆alkyl group,
R₇ represents a group selected from the group consisting of: 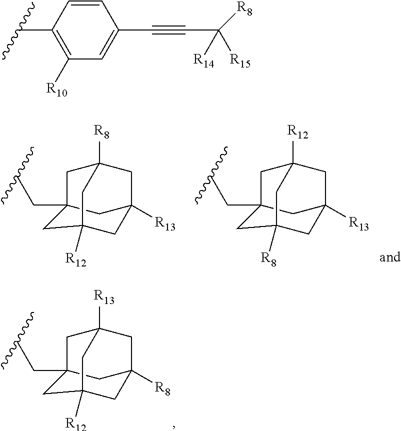
R₈ represents a group selected from the group consisting of: —NR′ₐR′_{b}; —O—X′₂—NR′ₐR′_{b}; and —X′₂—NR′ₐR′_{b},
R₁₀ represents fluorine,
R₁₂ and R₁₃, independently of one another, represent a hydrogen atom or a methyl group,
R₁₄ and R₁₅, independently of one another, represent a hydrogen or a methyl group,
X₁ and X₂ independently of one another, represent a linear or branched C₁-C₆alkylene group optionally substituted by one or two groups selected from the group consisting of trifluoromethyl, hydroxyl, a halogen, and a C₁-C₆alkoxy,
X′₂ represents a linear or branched C₁-C₆alkylene,
R′ₐ and R′_{b} independently of one another, represent a group selected from the group consisting of: hydrogen; a linear or branched C₁-C₆alkyl optionally substituted by one or two hydroxyl or C₁-C₆alkoxy groups; and a C₁-C₆alkylene-NR′_{d}R′ₑ;
or R′ₐ and R′_{b} form with the nitrogen atom carrying them a cycle B3,
R′d, R′ₑ independently of one another, represents a hydrogen or a linear or branched C₁-C₆alkyl group,
B₃ represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen and nitrogen, (iii) be substituted by one or two groups selected from the group consisting of: fluorine, bromine, chlorine, a linear or branched C₁-C₆alkyl, hydroxyl, and oxo.

21. The compound of any one of claims 1-20, wherein R₆ represents —X₂—O—R₇, and R₇ represents the following group:

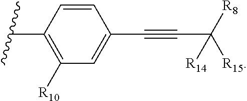

22. The compound of any one of claims 1-20, wherein R₆ represents a heteroarylene-R₇ group optionally substituted by a linear or branched C₁-C₆alkyl group, and R₇ represents a group selected from the group consisting of:

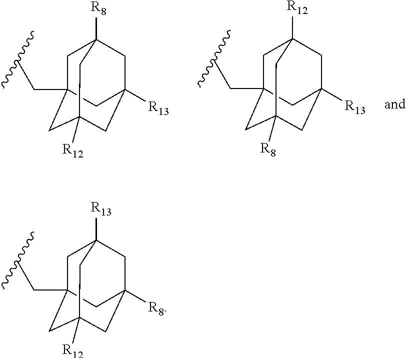

23. The compound of any one of claims 1-22, wherein B₃ represents a C₃-C₆heterocycloalkyl group selected from the group consisting of a pyrrolidinyl group, a piperidinyl group, a piperazinyl group, a morpholinyl group, an azepanyl group, and a 4,4-difluoropiperidin-1-yl group.

24. The compound of claim 23, wherein B₃ represents a pyrrolidinyl group or a piperazinyl group.

25. The compound of claim 23, wherein B₃ represents a piperazinyl group.

26. The compound of any one of claims 1-22, wherein R₈ represents a group selected from the group consisting of: 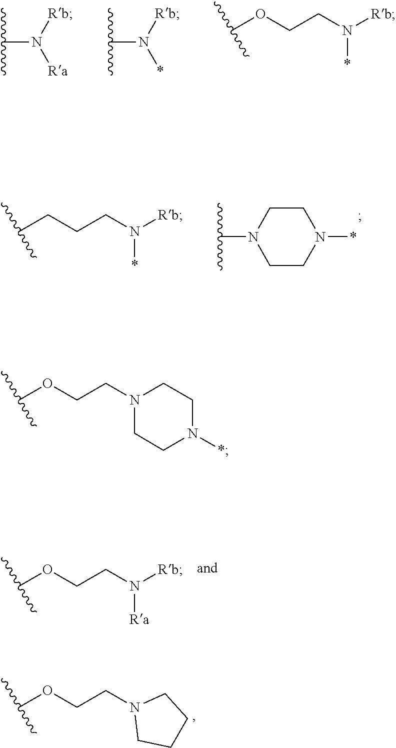
wherein:
-* is a bond to the linker.

27. The compound of any one of claims 1-22, wherein R₈ represents a group selected from the group consisting of: 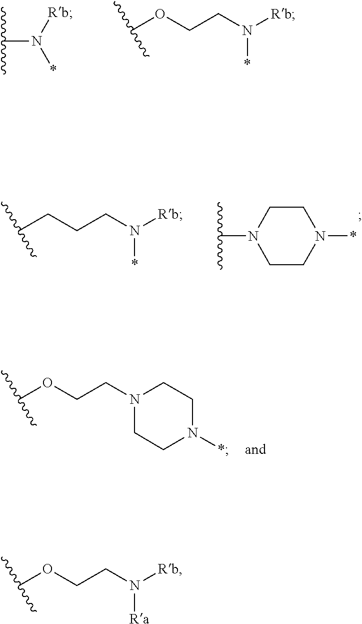
wherein:
-* is a bond to the linker.

28. The compound of claim 19, wherein:
for formula (IB) or (IC-1), R₃ represents a group selected from the group consisting of: hydrogen; a linear or branched C₁-C₆alkyl; —X₁—NRₐR_{b}; —X₁—N₃ and
—X₁-≡CH,
for formula (IIB) or (IIC-1), Z₁ represents a bond, and R₃ represents hydrogen,
for formula (IC-1), G is selected from the group consisting of —C(O)OH and —C(O)N(CH₃)₂;
for formula (IIC-1), G is selected from the group consisting of —C(O)NHS(O)₂H, —C(O)NH₂, —C(O)NHCH₃, —C(O)NHC(CH₃)₂, —C(O)N(CH₃)₂, —C(O)OH, and —CH₂OH;
R₆ represents a linear or branched —C₁-C₆alkylene-R₈ group, —X₂—O—R₇ or a heteroarylene-R₇ group optionally substituted by a linear or branched C₁-C₆alkyl group,
R₇ represents a group selected from the group consisting of: 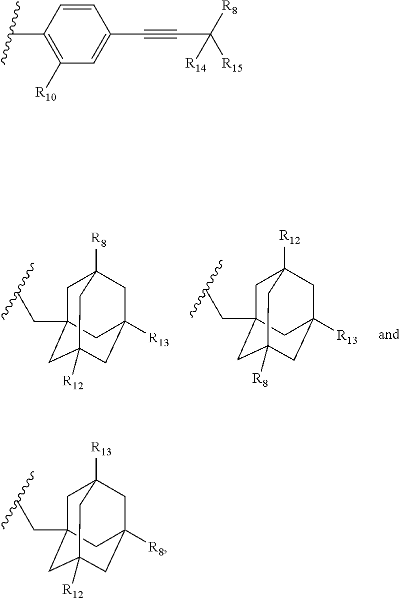
R₈ represents a group selected from the group consisting of: —NR′ₐR′_{b}; and —O—X′₂—NR′ₐR′_{b},
R₁₀ represents fluorine,
R₁₂ and R₁₃, independently of one another, represent a hydrogen atom or a methyl group,
R₁₄ and R₁₅, independently of one another, represent a hydrogen or a methyl group,
X₁ and X₂ independently of one another, represent a linear or branched C₁-C₆alkylene group optionally substituted by one or two groups selected from the group consisting of trifluoromethyl, hydroxyl, a halogen, and C₁-C₆alkoxy,
X′₂ represents a linear or branched C₁-C₆alkylene,
R′ₐ and R′_{b} independently of one another, represent a group selected from the group consisting of: hydrogen; a linear or branched C₁-C₆alkyl optionally substituted by one or two hydroxyl or C₁-C₆alkoxy groups; and a C₁-C₆alkylene-NR′_{d}R′ₑ;
or R′ₐ and R′_{b} form with the nitrogen atom carrying them a cycle B₃,
R′_{d}, R′ₑ independently of one another, represents a hydrogen or a linear or branched C₁-C₆alkyl group,
B₃ represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen and nitrogen, (iii) be substituted by one or two groups selected from the group consisting of: fluorine, bromine, chlorine, linear or a branched C₁-C₆alkyl, hydroxyl, and oxo.

29. The compound of claim 20, wherein:
for formula (IB) or (IC), R₃ represents a group selected from the group consisting of: hydrogen; a linear or branched C₁-C₆alkyl; —X₁—N₃ and —X₁-≡CH, for formula (IIB) or (IIC), Z₁ represents a bond, and R₃ represents hydrogen,
R₆ represents a linear or branched —C₁-C₆alkylene-R₈ group, —X₂—O—R₇ or a heteroarylene-R₇ group optionally substituted by a linear or branched C₁-C₆alkyl group,
R₇ represents a group selected from the group consisting of: 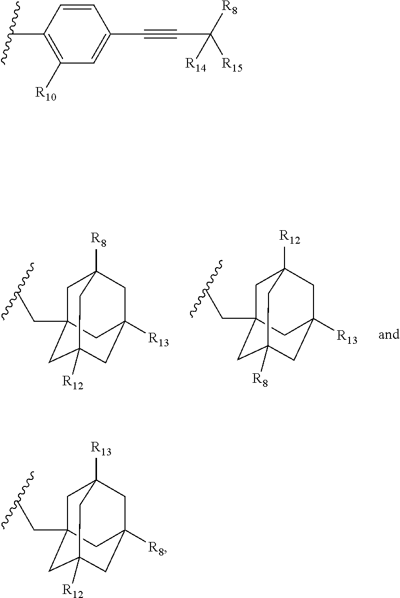
R₈ represents a group selected from the group consisting of: —NR′ₐR′_{b}; and —O—X′₂—NR′ₐR′_{b},
R₁₀ represents fluorine,
R₁₂ and R₁₃, independently of one another, represent a hydrogen atom or a methyl group,
R₁₄ and R₁₅, independently of one another, represent a hydrogen or a methyl group,
X₁ and X₂ independently of one another, represent a linear or branched C₁-C₆alkylene group optionally substituted by one or two groups selected from the group consisting of trifluoromethyl, hydroxyl, a halogen, and C₁-C₆alkoxy,
X′₂ represents a linear or branched C₁-C₆alkylene,
R′ₐ and R′_{b} independently of one another, represent a group selected from the group consisting of: hydrogen; a linear or branched C₁-C₆alkyl optionally substituted by one or two hydroxyl or C₁-C₆alkoxy groups; and a C₁-C₆alkylene-NR′_{d}R′ₑ;
or R′ₐ and R′_{b} form with the nitrogen atom carrying them a cycle B₃,
R′_{d}, R′ₑ independently of one another, represents a hydrogen or a linear or branched C₁-C₆alkyl group,
B₃ represents a C₃-C₆heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen and nitrogen, (iii) be substituted by one or two groups selected from the group consisting of: fluorine, bromine, chlorine, linear or a branched C₁-C₆alkyl, hydroxyl, and oxo.

30. The compound of claim 28 or 29, wherein B₃ represents pyrrolidinyl group or a piperazinyl group.

31. The compound of claim 28 or 29, where B₃ represents a piperazinyl group.

32. The compound of claim 28 or 29, wherein R₈ represents a group selected from the group consisting of: 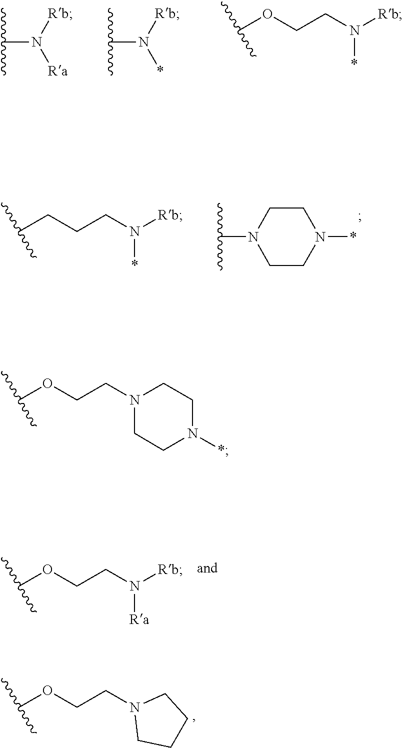
wherein:
-* is a bond to the linker.

33. The compound of claim 28 or 29, wherein R₈ represents a group selected from the group consisting of: 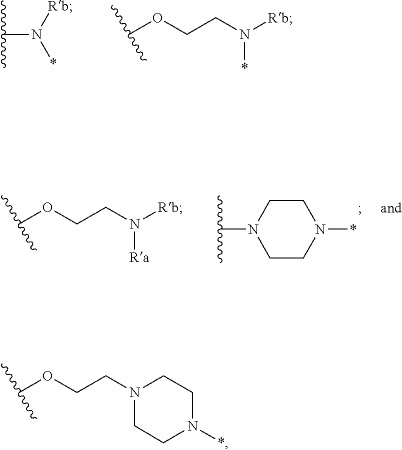
wherein:
-* is a bond to the linker.

34. The compound of any one of claims 1 to 33, wherein 0 represents any one of the following attached to L: 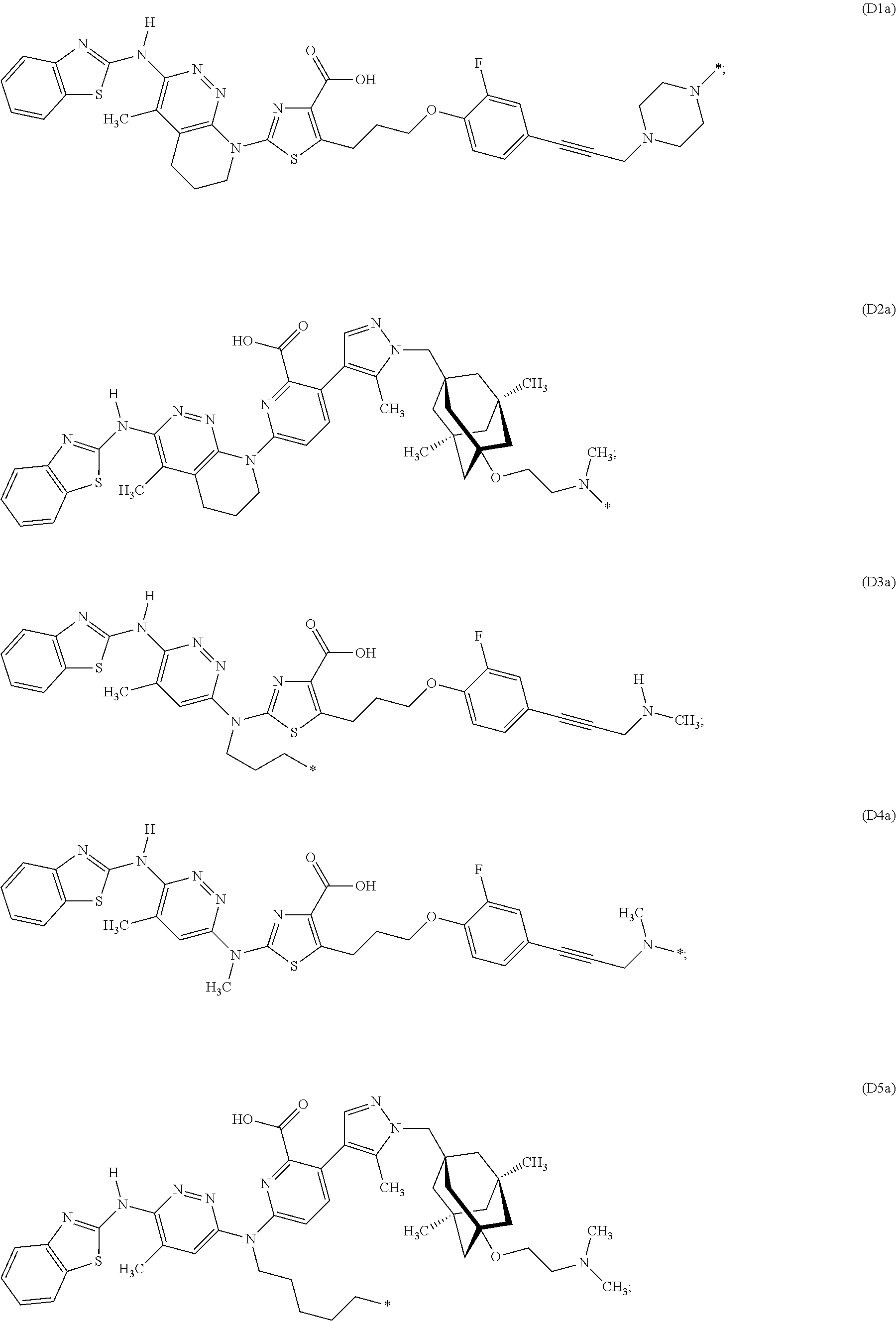 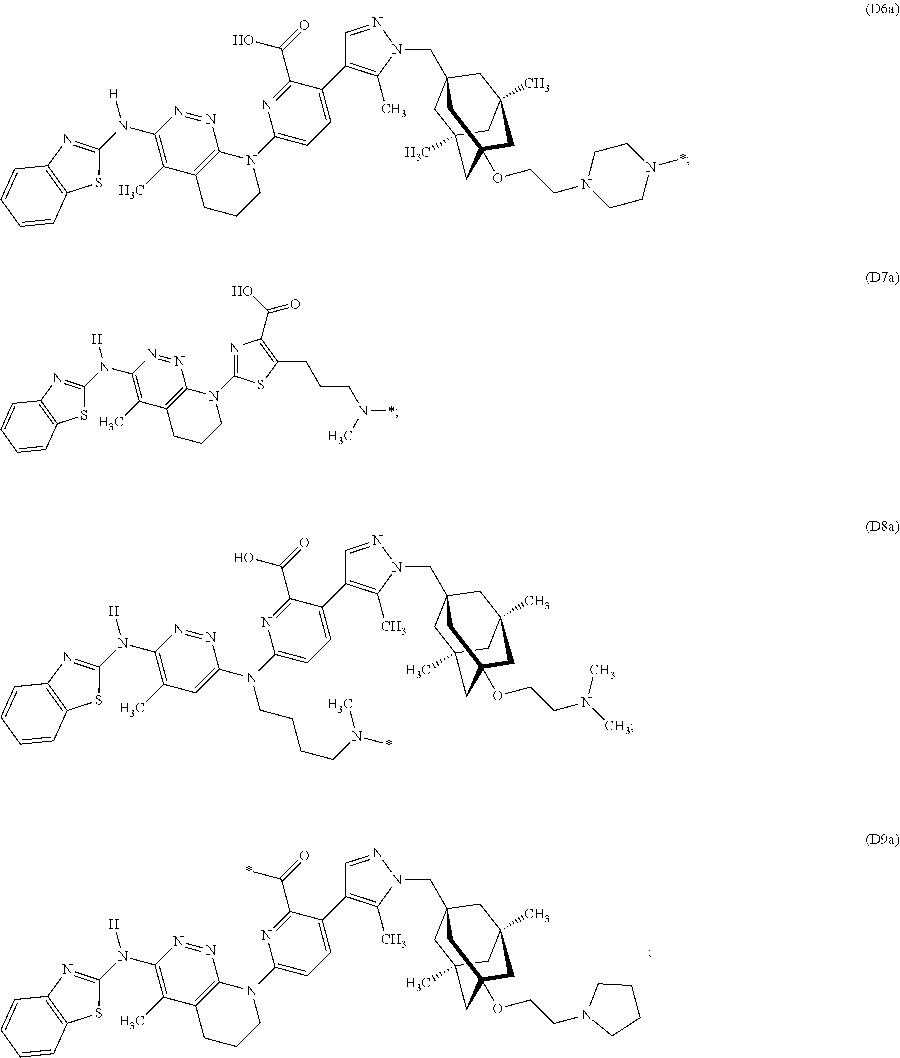 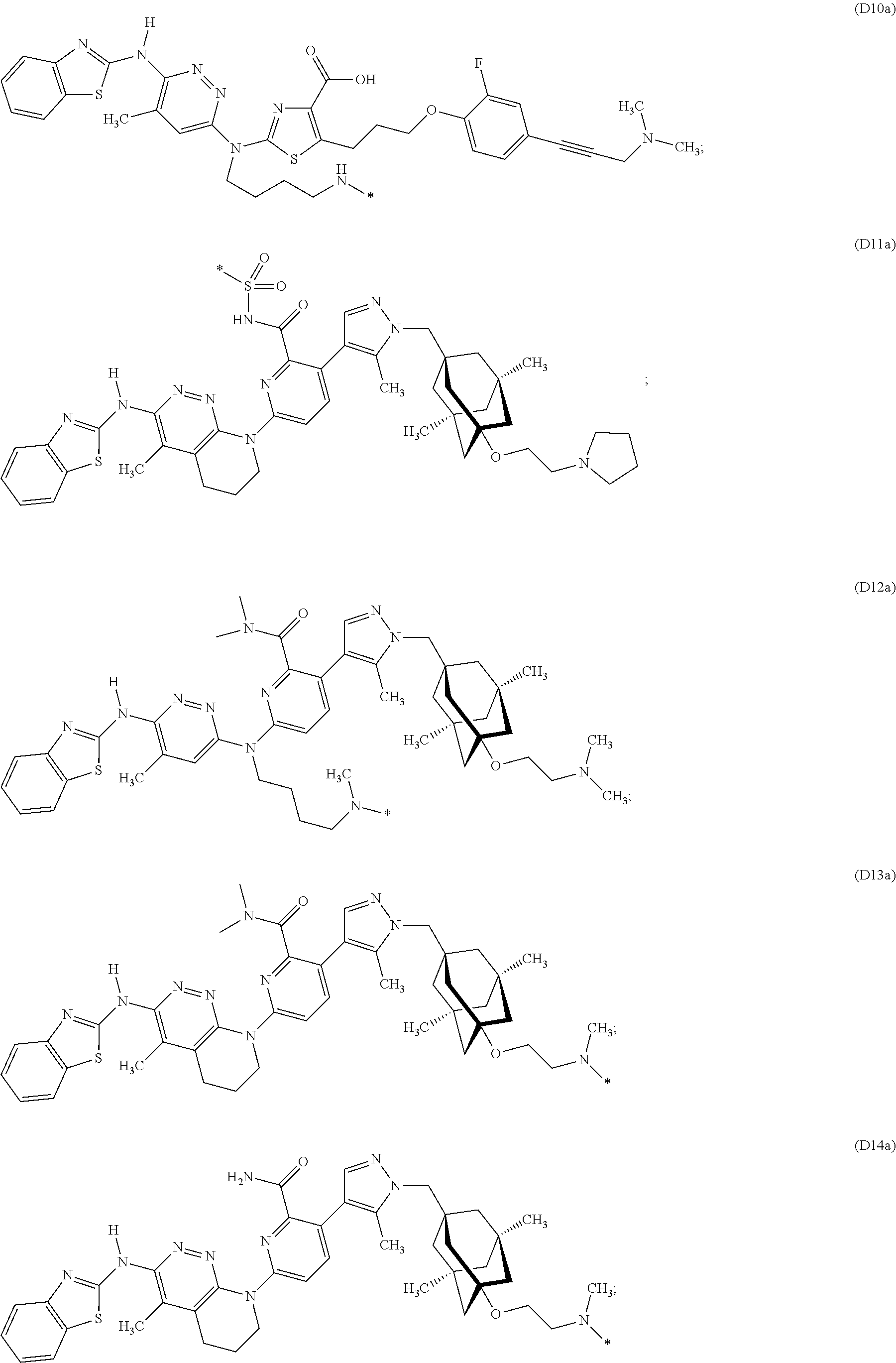 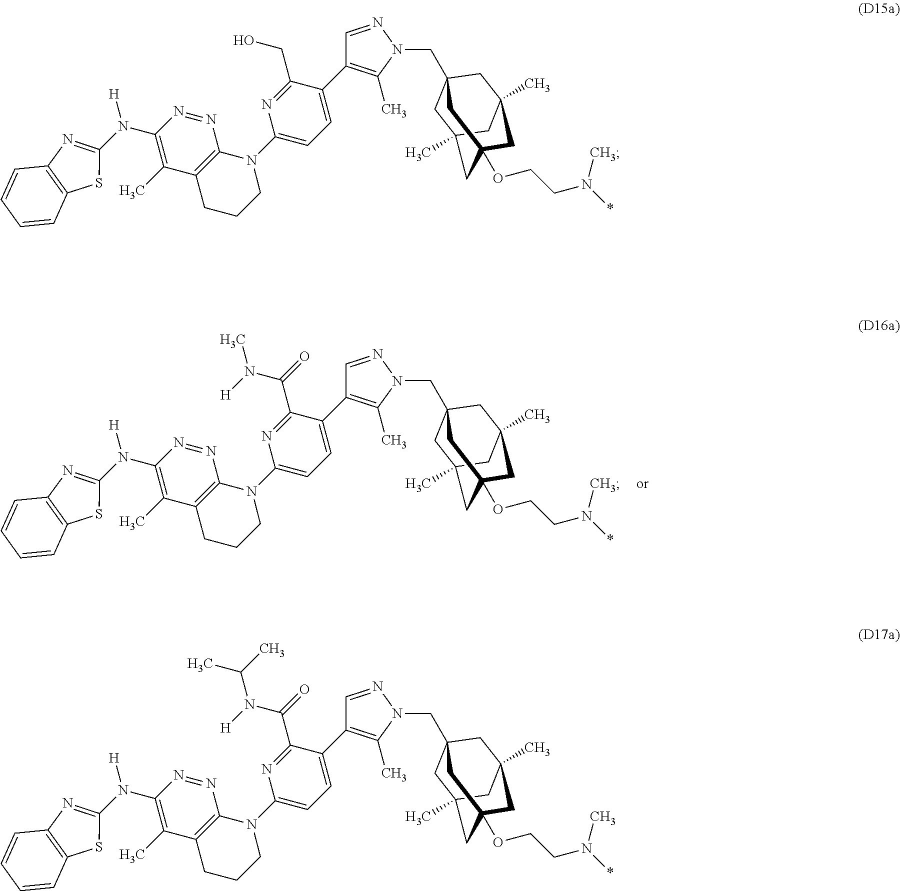
or an enantiomer, a diastereoisomer, and/or a pharmaceutically acceptable salt of any one of the foregoing, wherein:
—* is a bond to the linker.

35. The compound of claim 1, wherein D-L comprises a formula selected from the group consisting of: 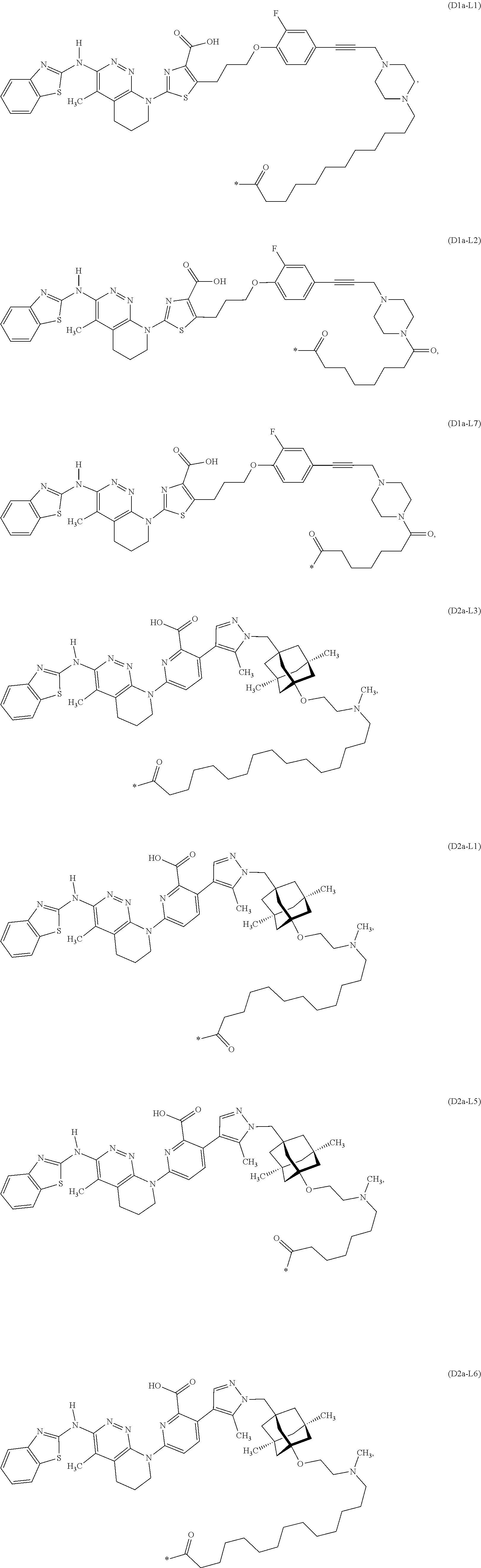 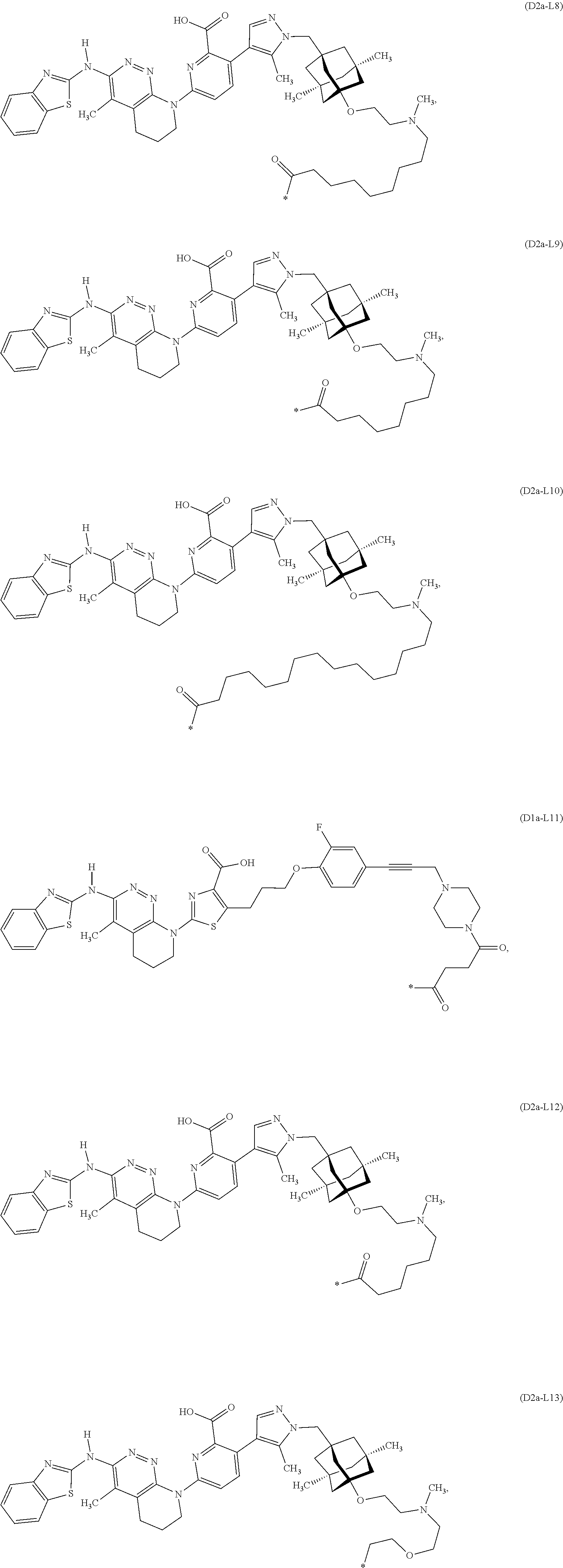 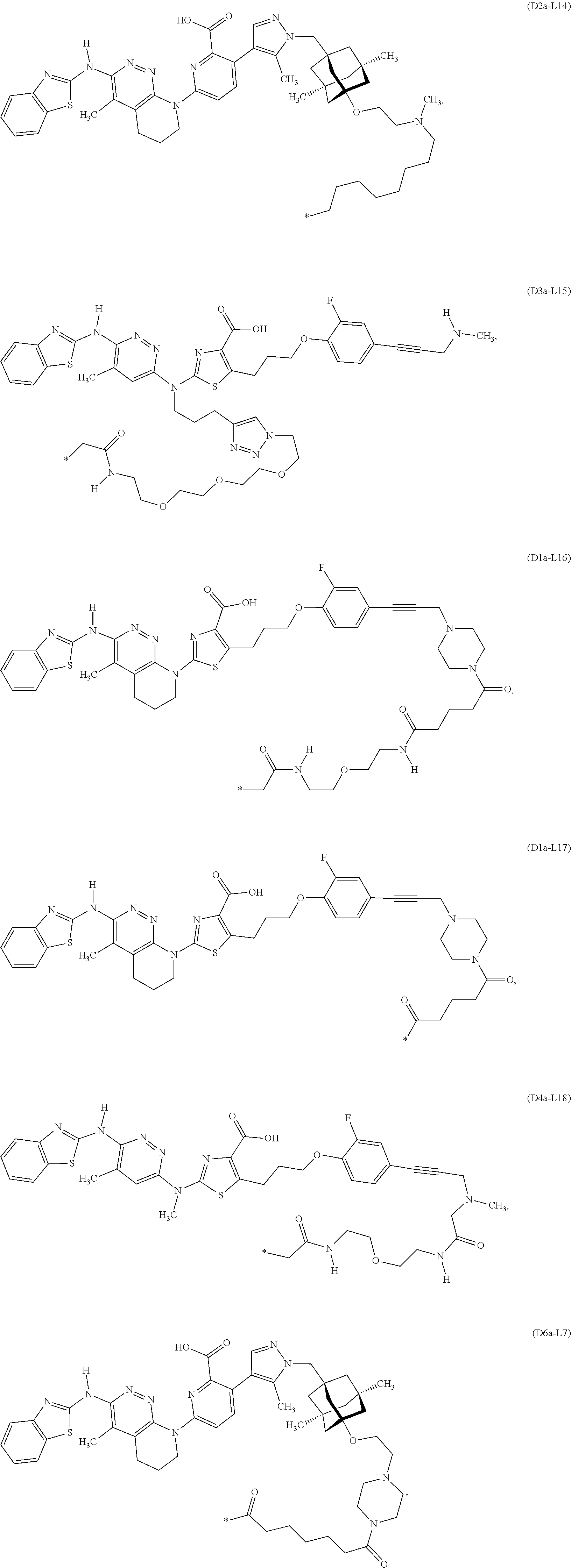 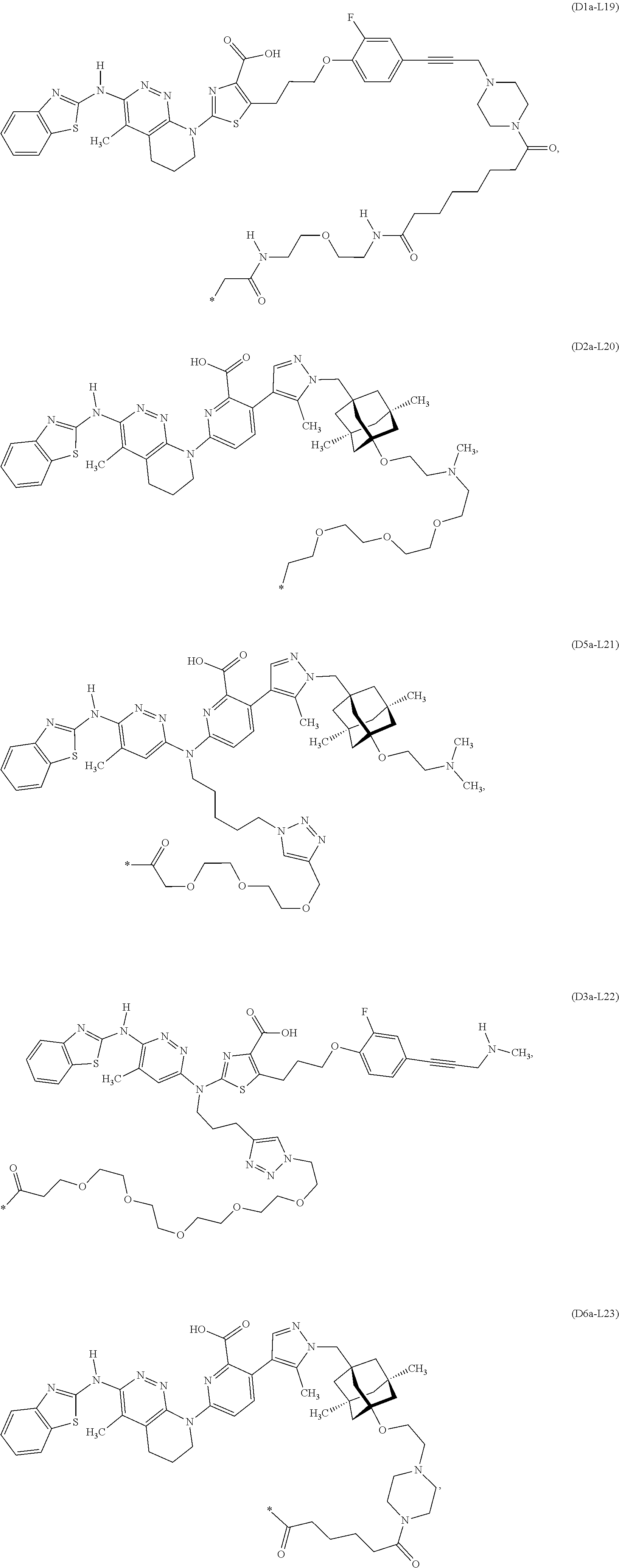 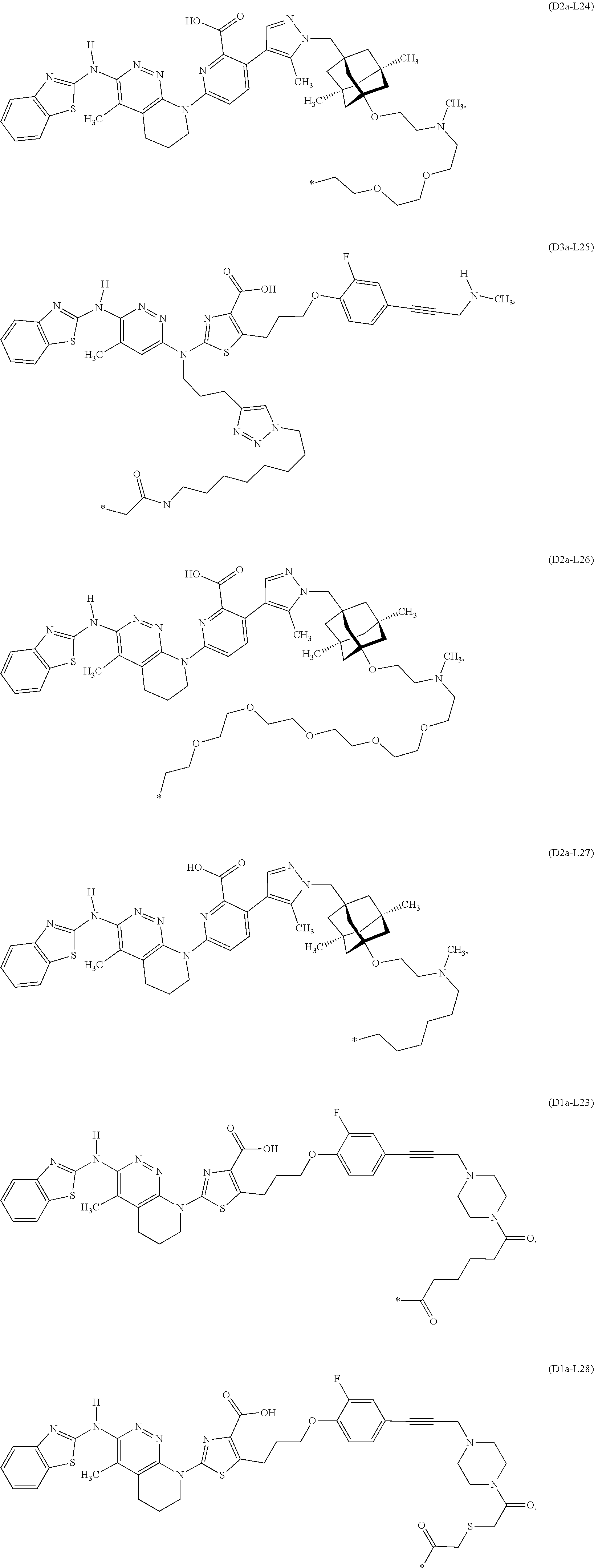 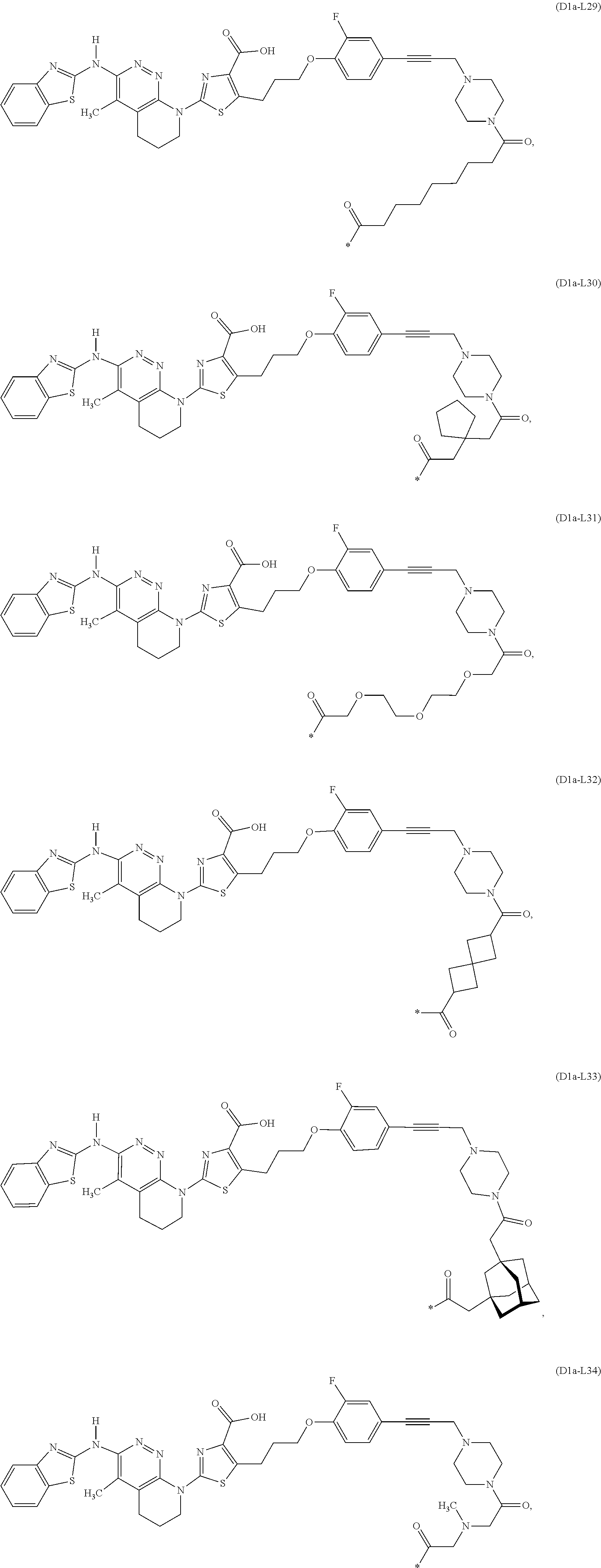 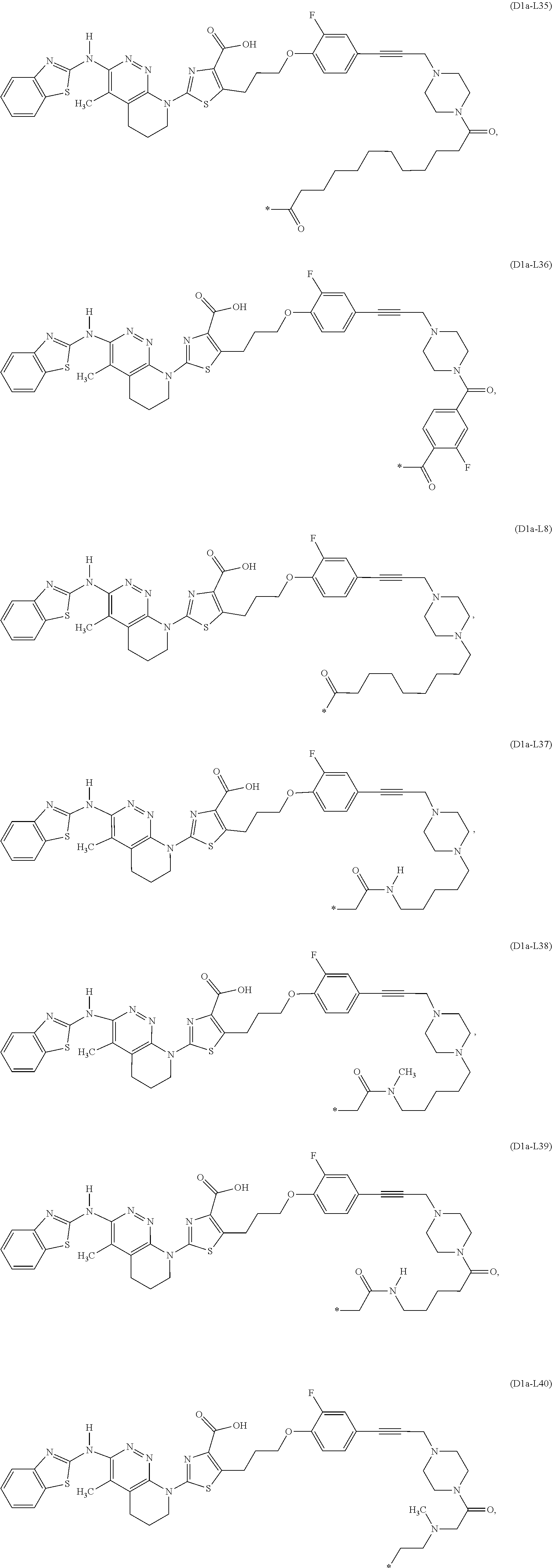 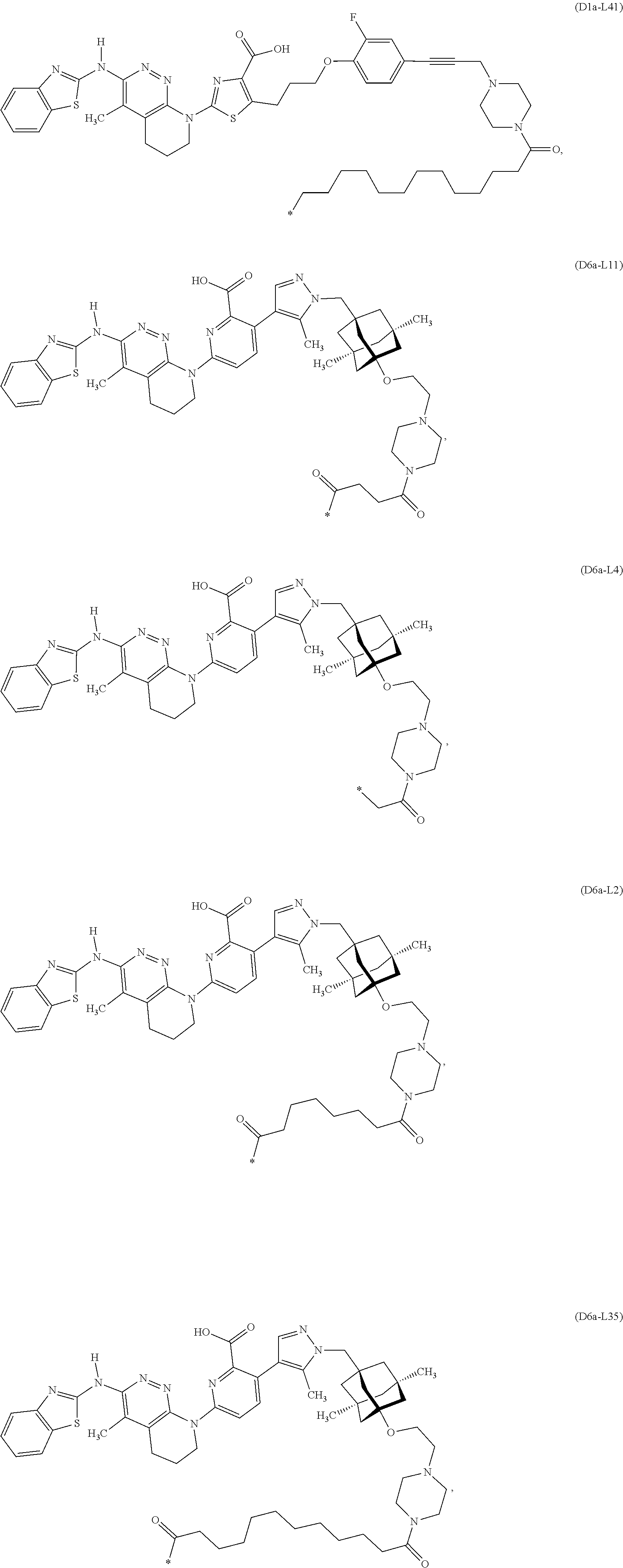 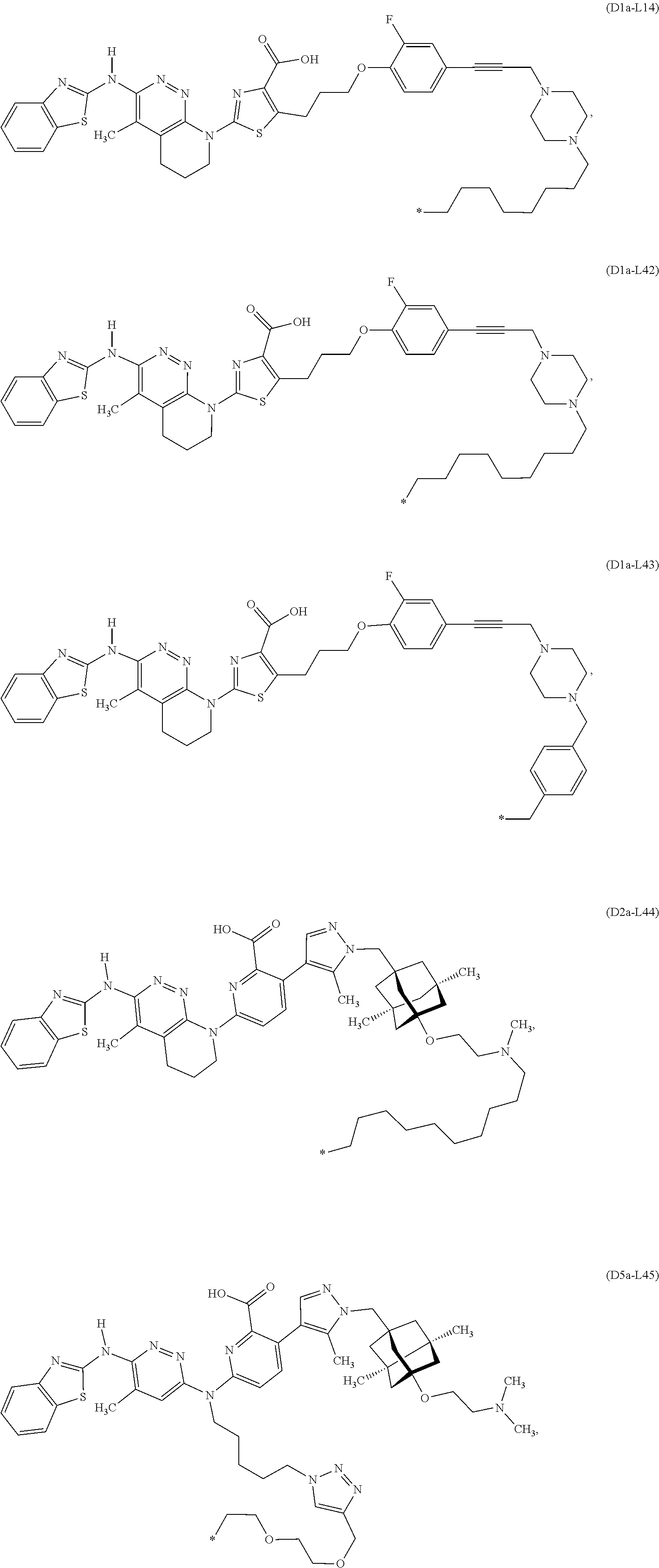 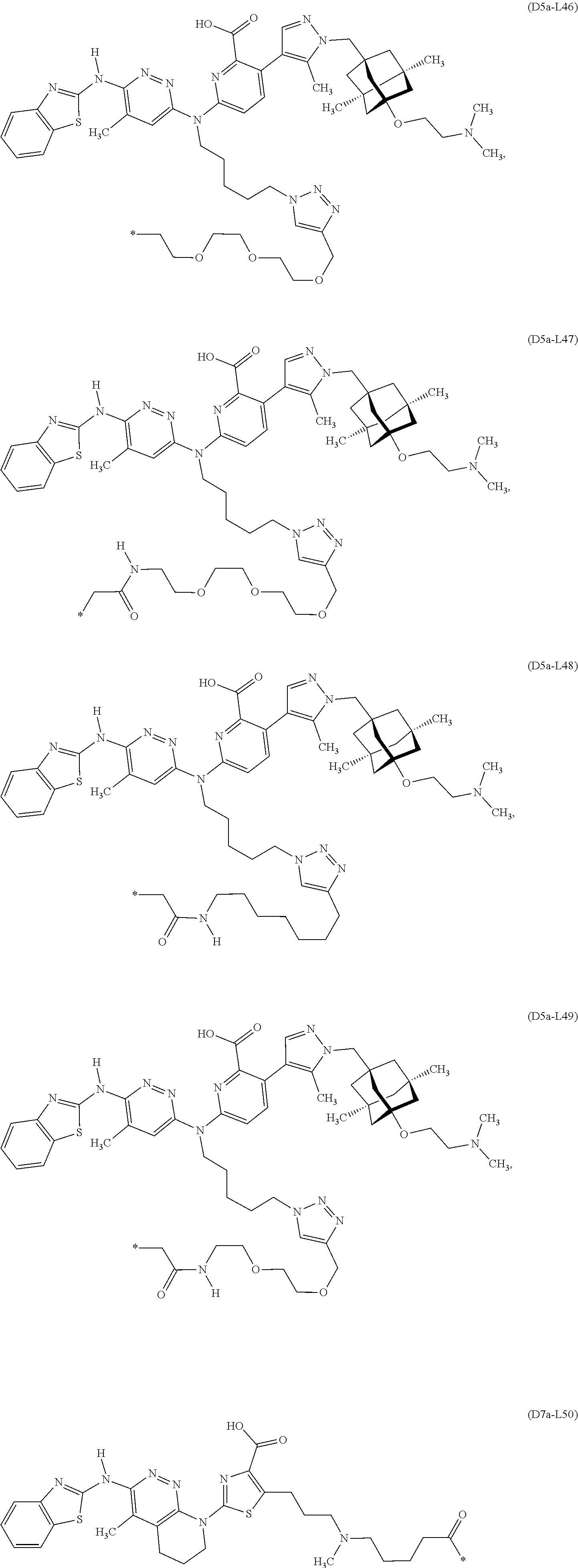 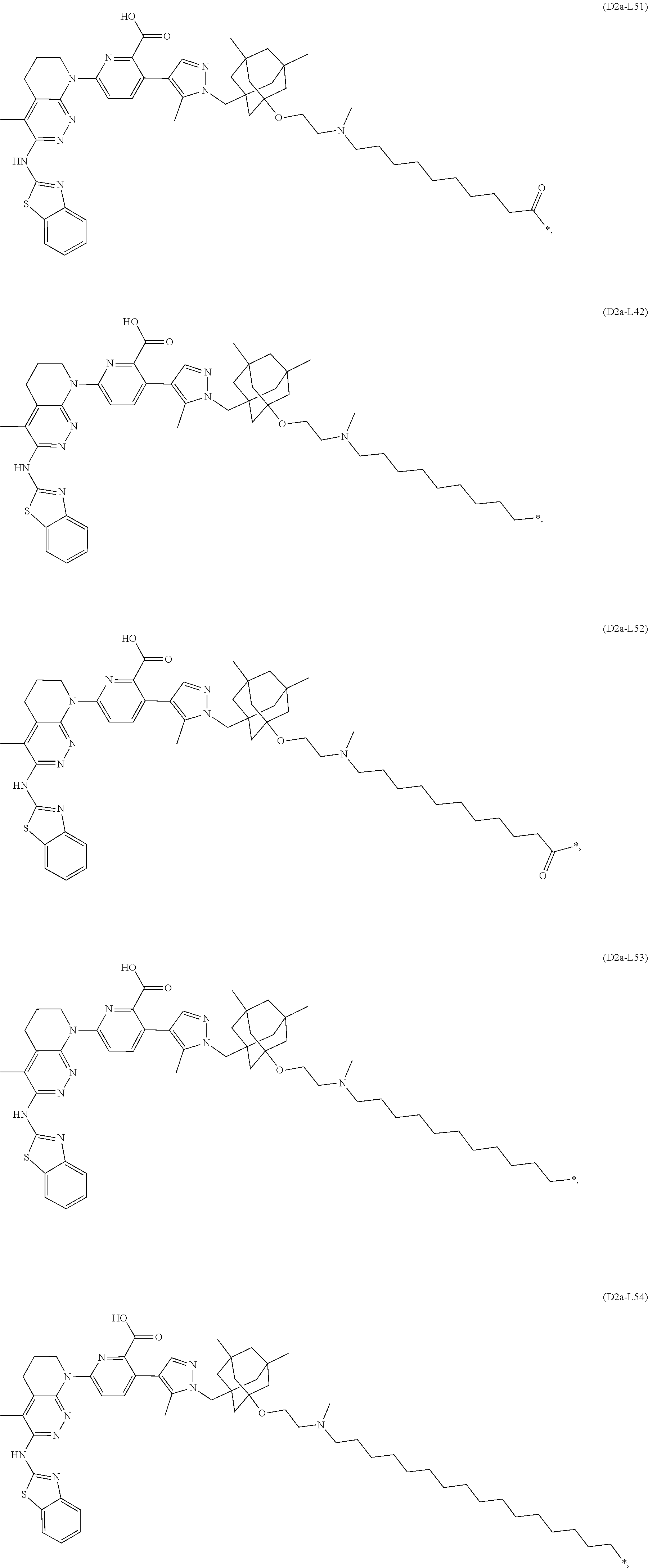 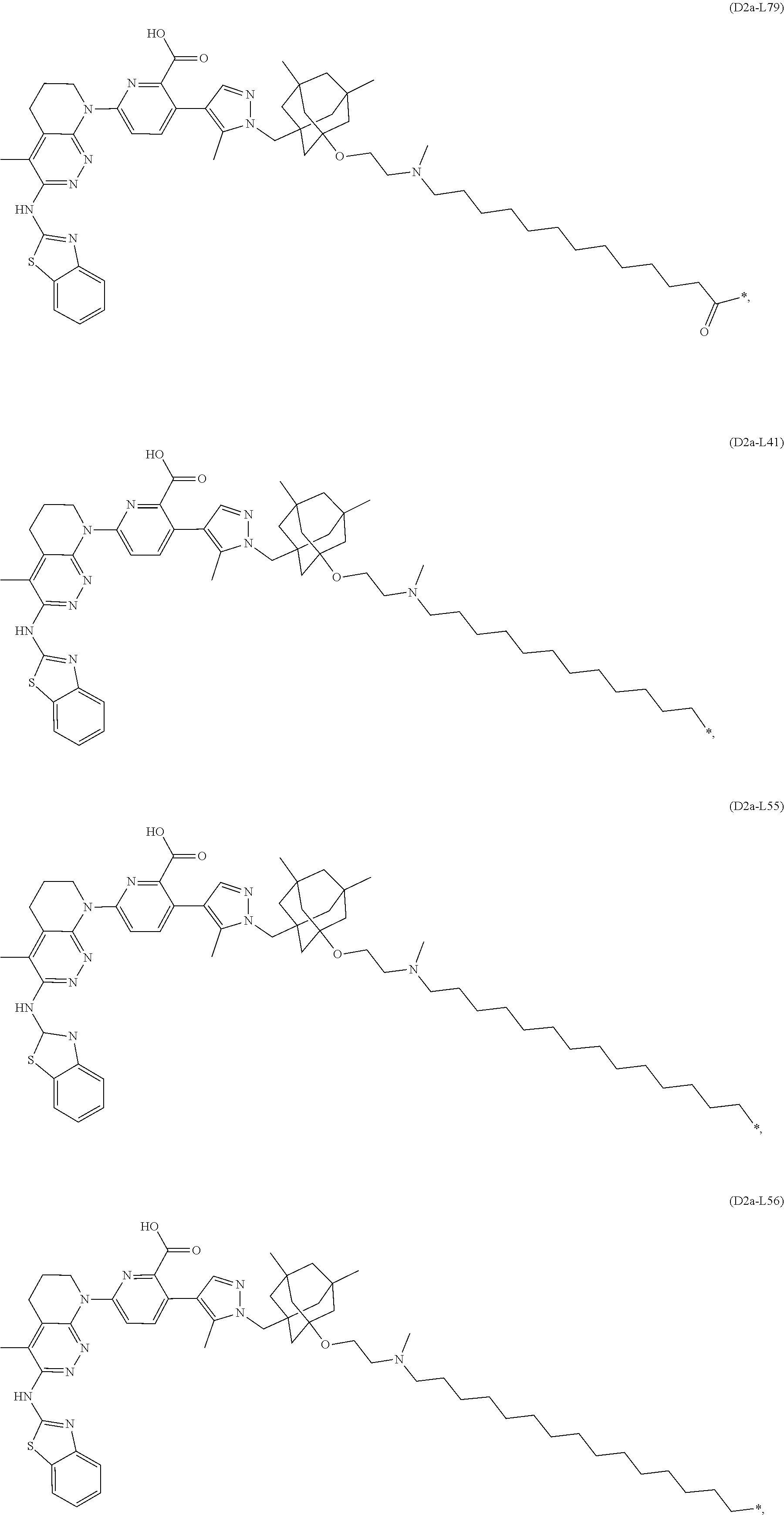 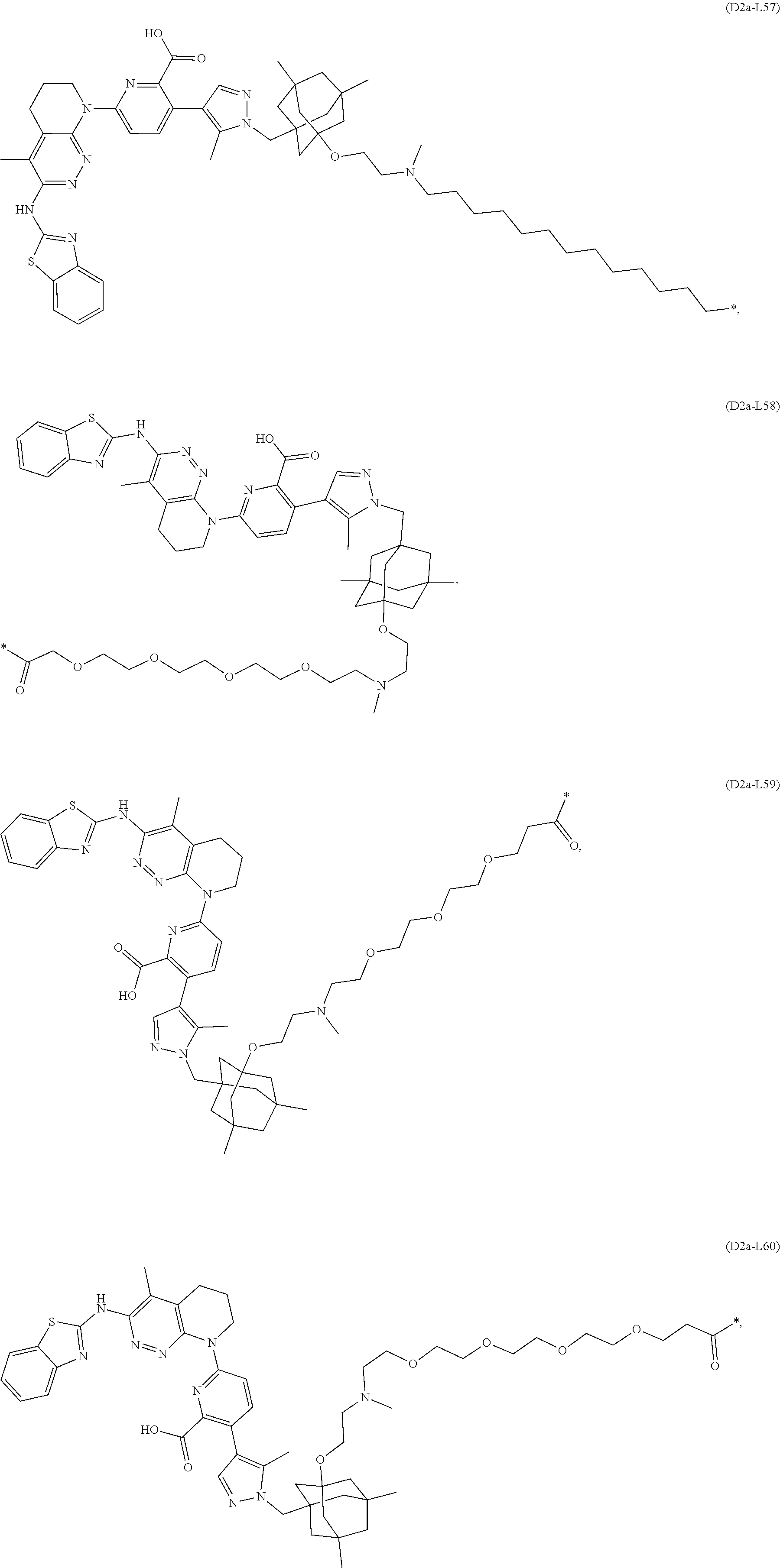 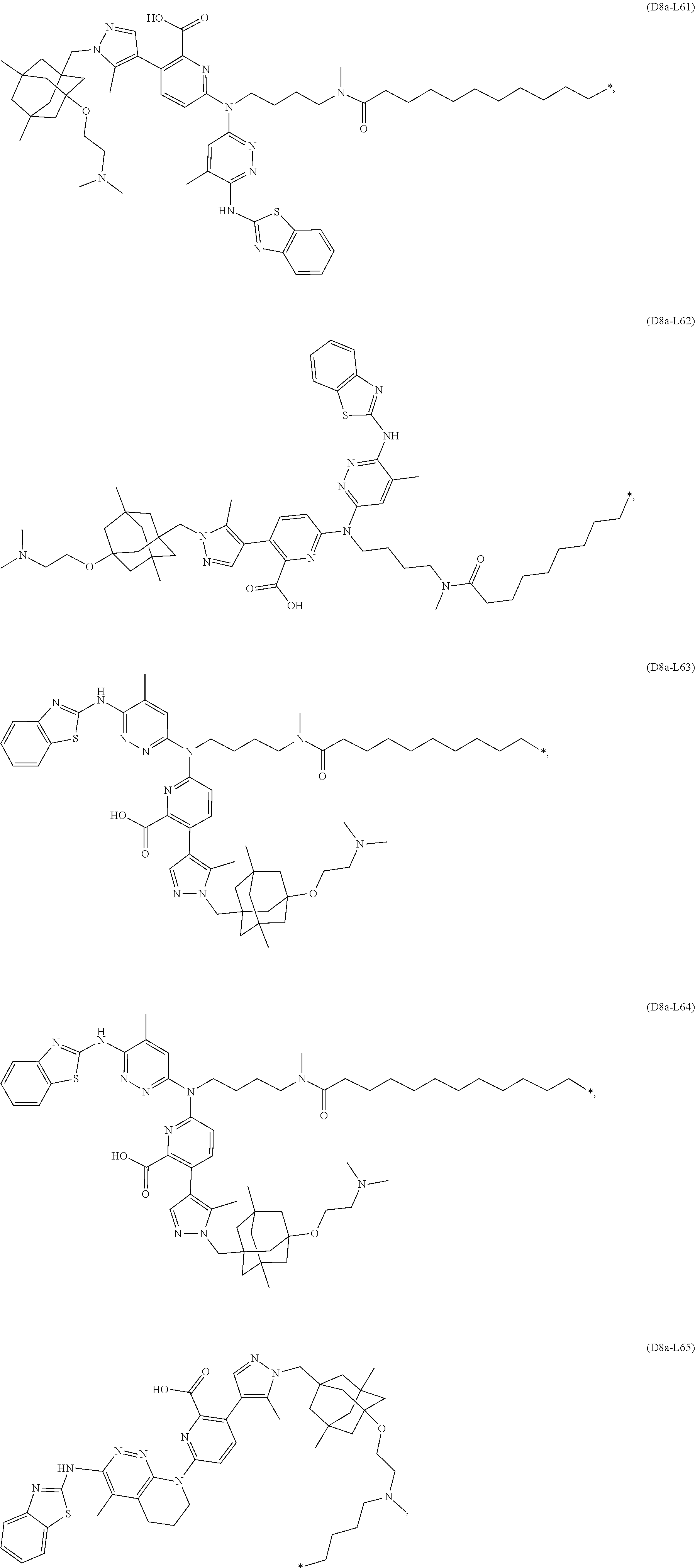 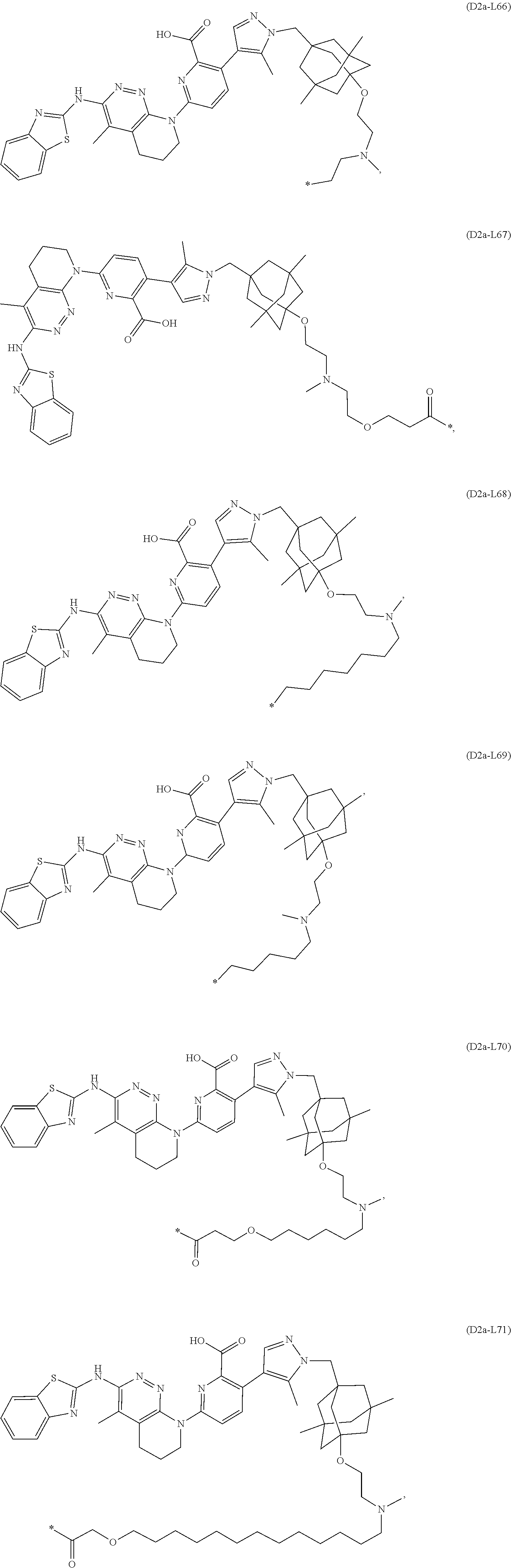 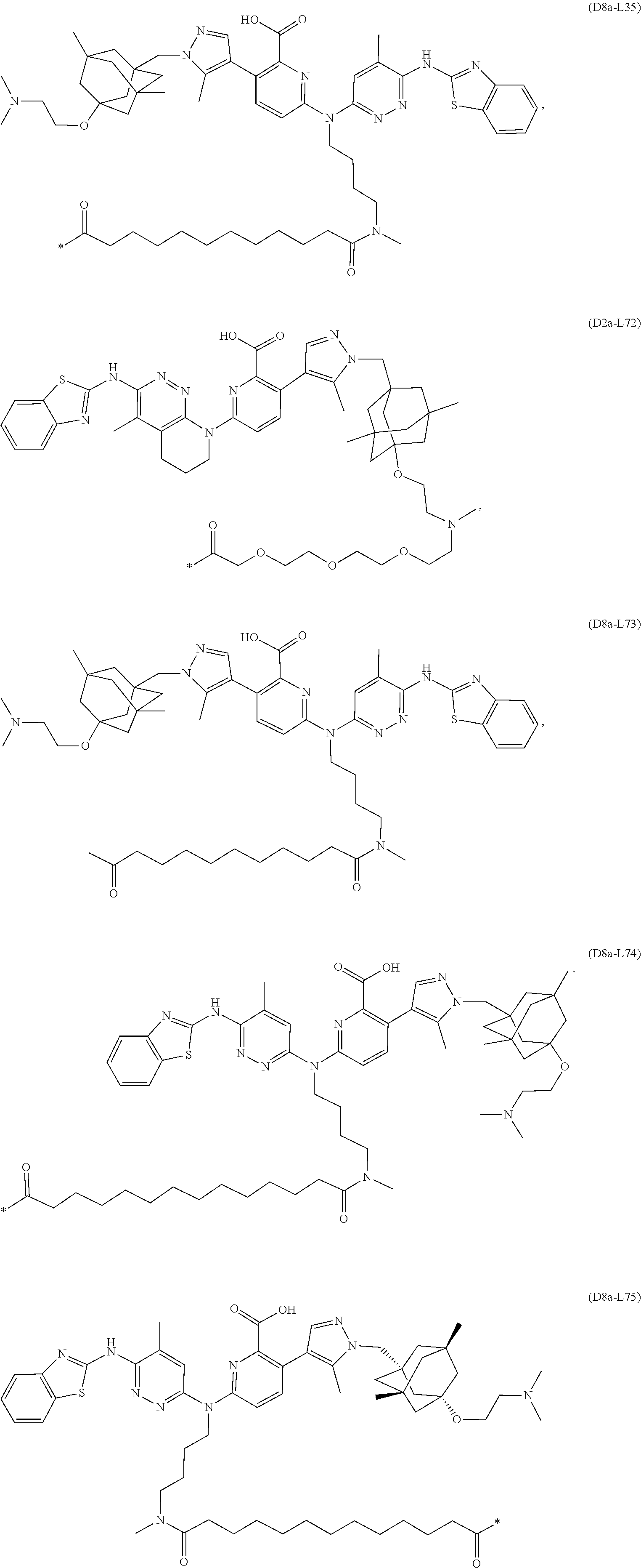 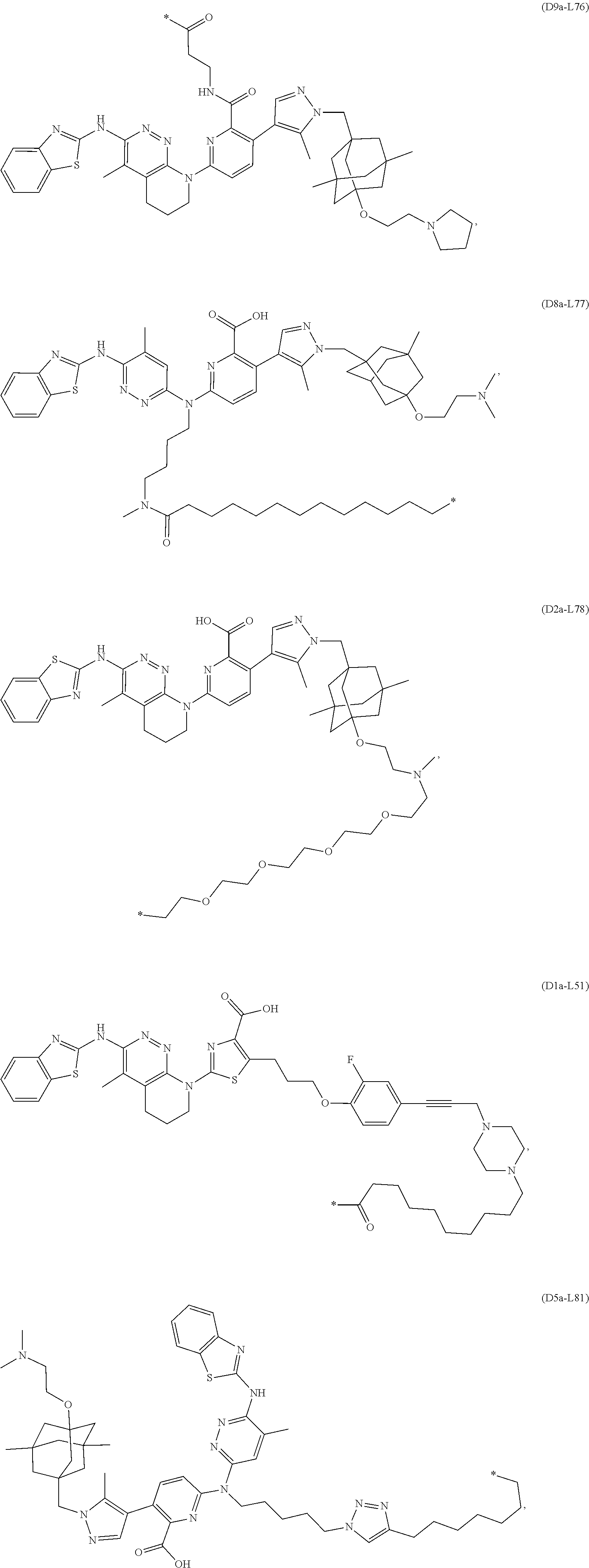 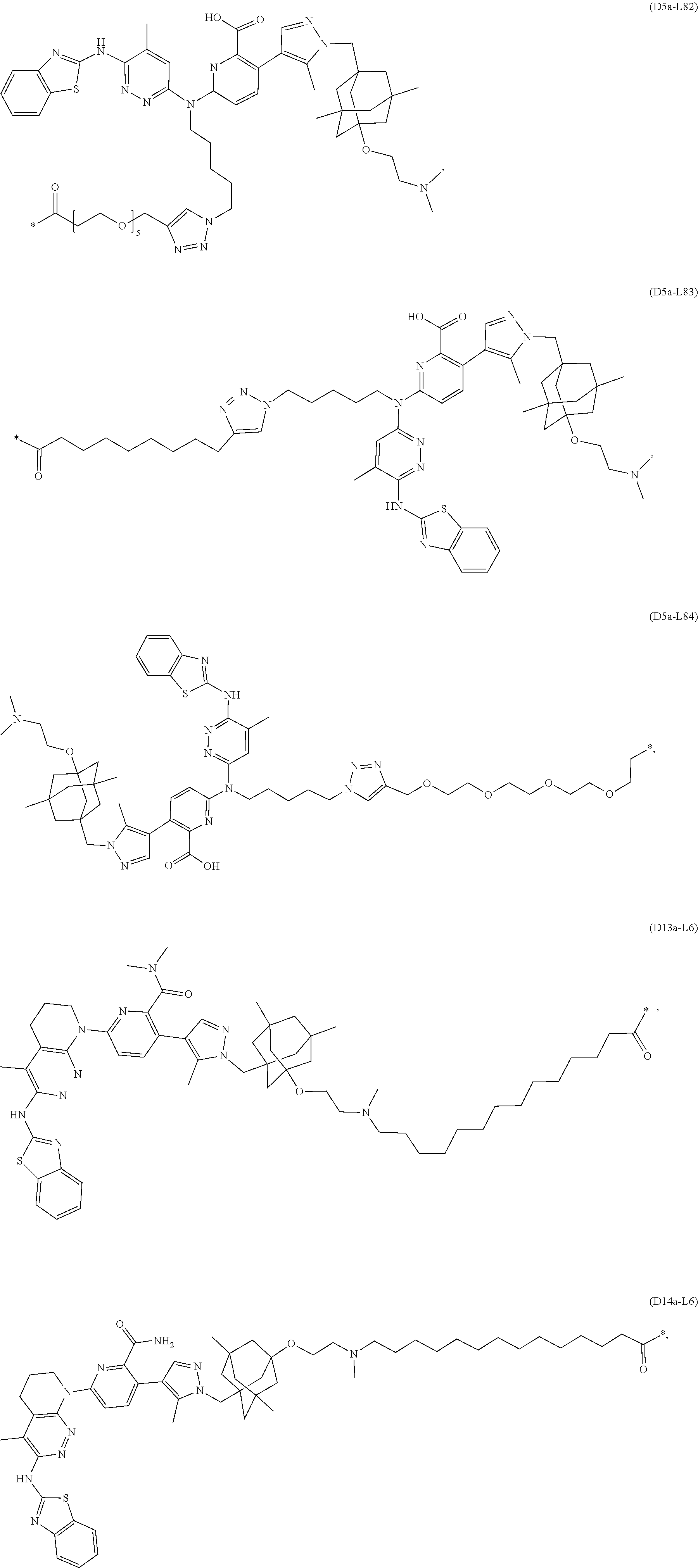 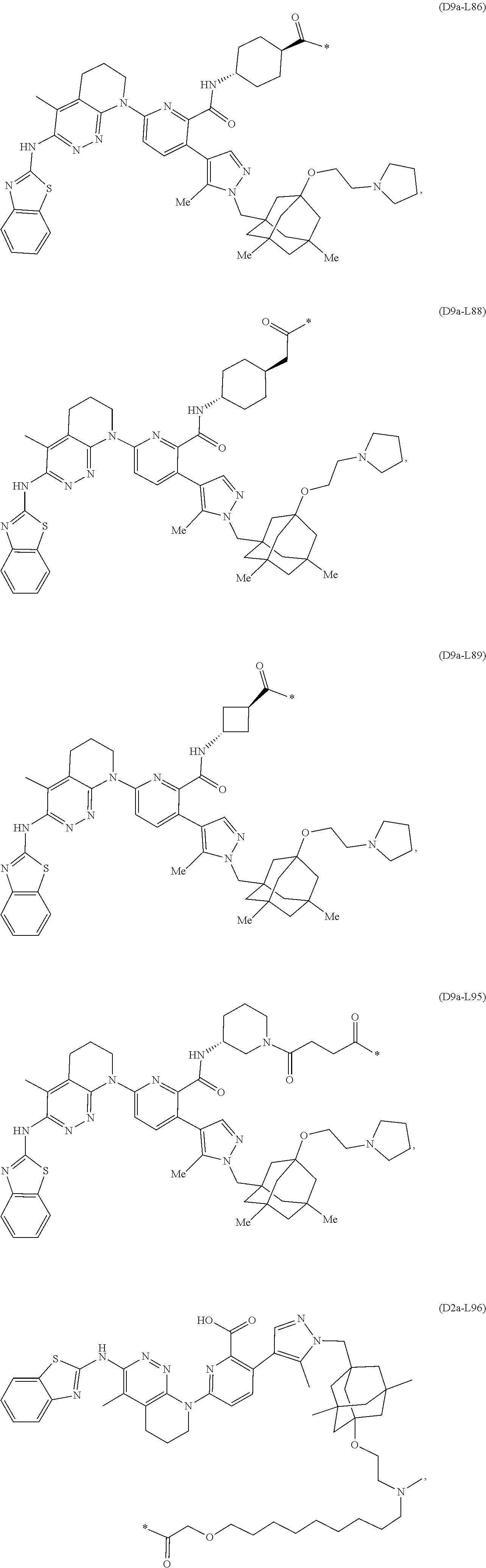 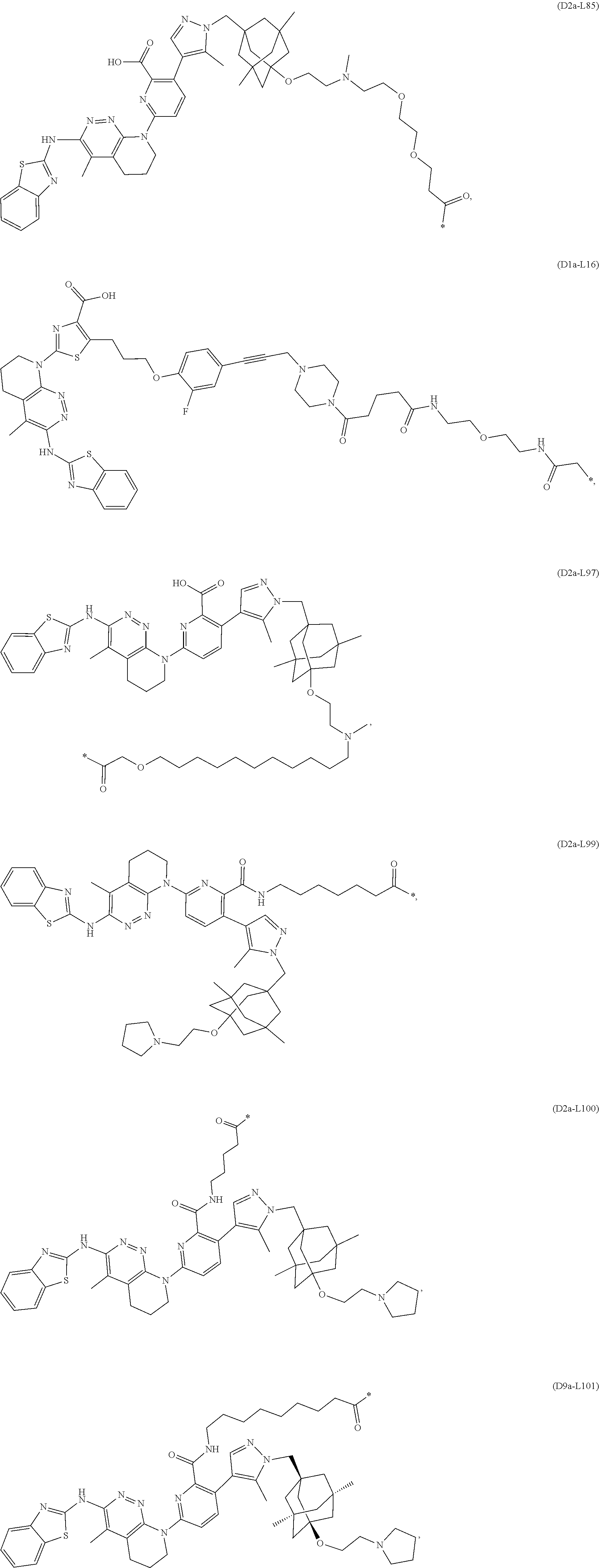 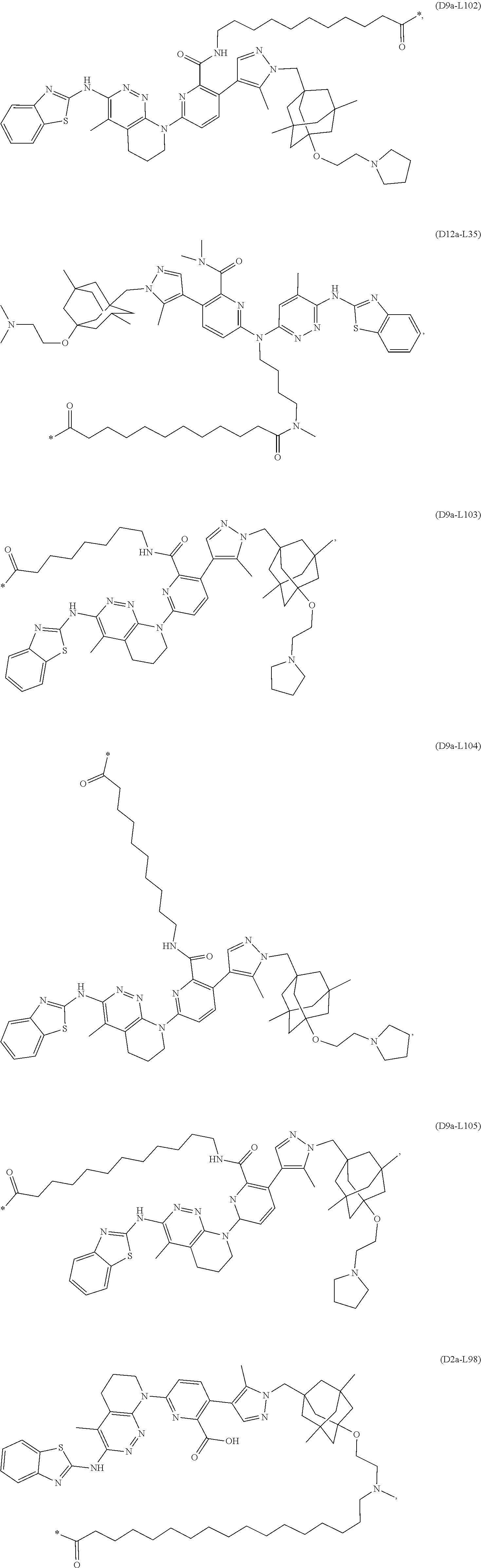 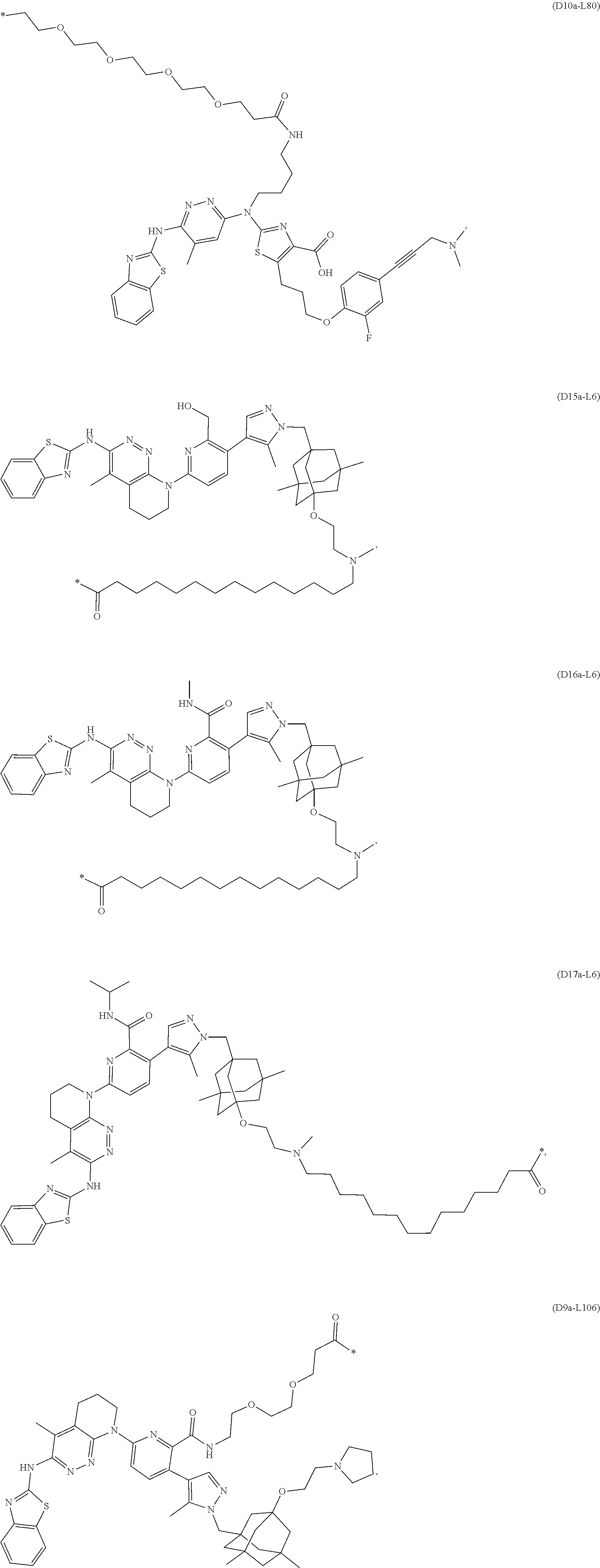 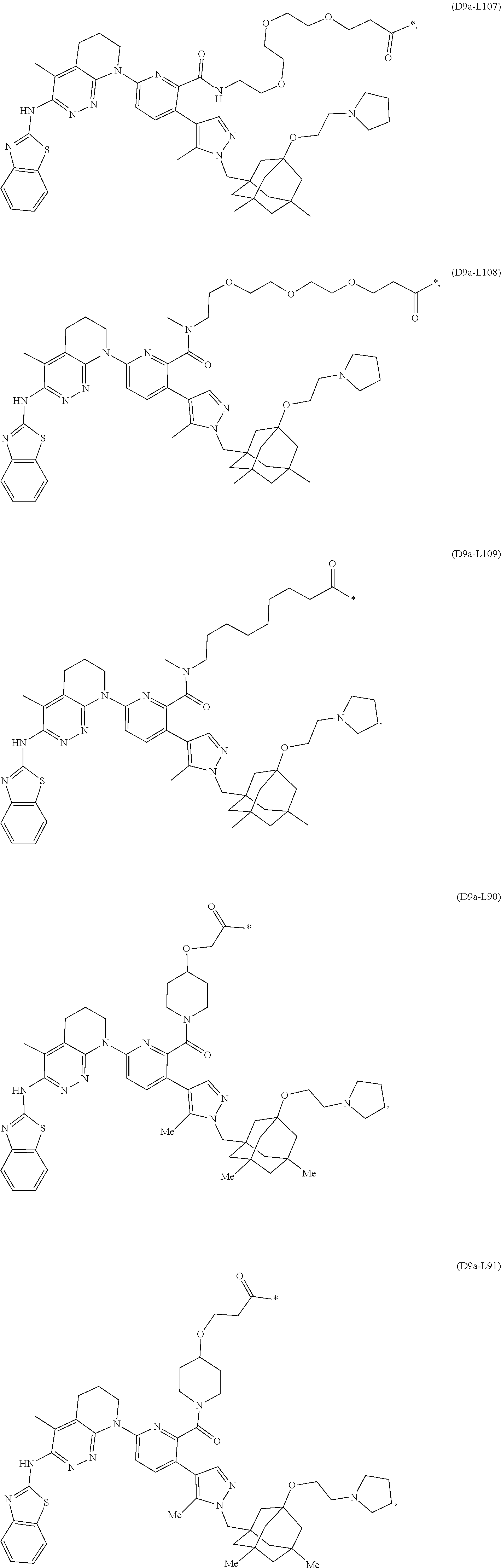 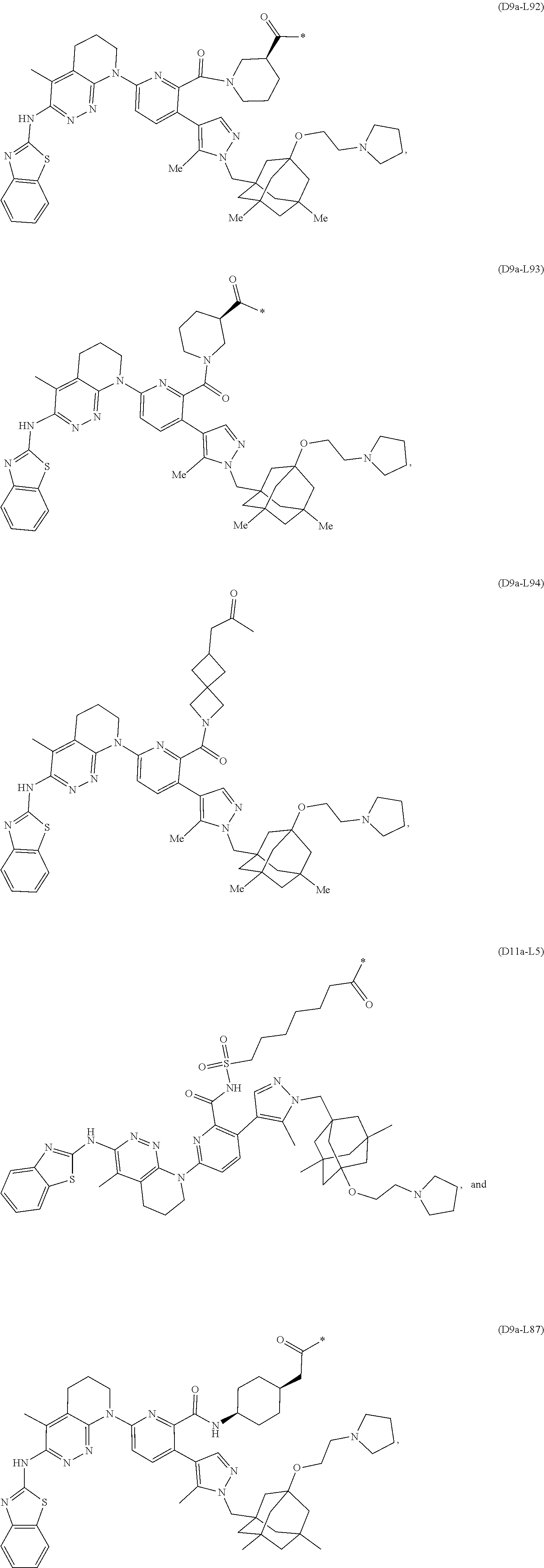
wherein -* is a bond to the DSM.

36. The compound of any one of claims 1 to 35, wherein the degradation signaling compound is a E3 ligase recognition agent.

37. The compound of any one of claims 1 to 35, wherein the degradation signaling compound is a VHL ligand, a thalidomide cereblon binder, or an inhibitor of apoptosis (IAP) E3 ligases.

38. The compound of any one of claims 1 to 35, wherein DSM represents any one of the following attached to L: 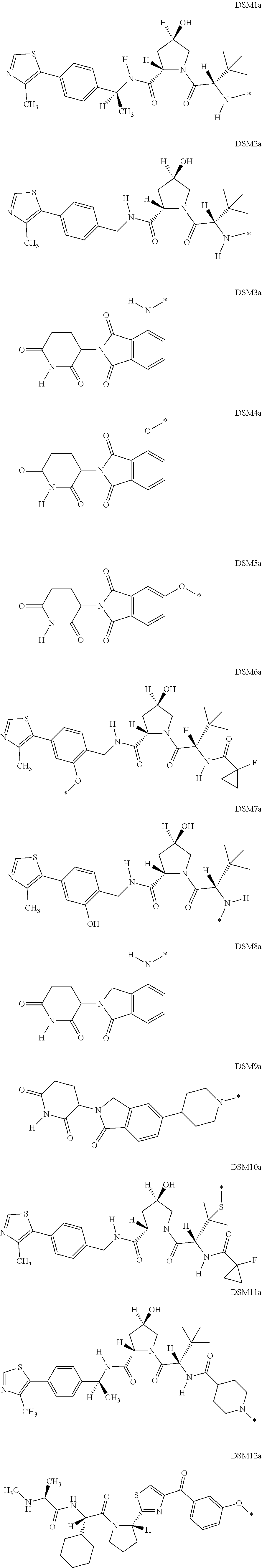
or an enantiomer, a diastereoisomer, and/or a pharmaceutically acceptable salt of any one of the foregoing, wherein -* represents a bond to the linker (L).

39. The compound of any one of claims 1 to 38, wherein DSM represents the following attached to L: 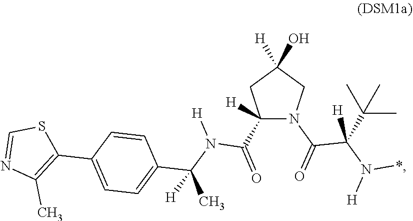
or an enantiomer, a diastereoisomer, and/or a pharmaceutically acceptable salt of any one of the foregoing,
wherein -* represents a bond to the linker (L).

40. The compound of claim 1, wherein the compound is any one of the compounds in Table 7 or an enantiomer, a diastereoisomer, and/or a pharmaceutically acceptable salt of any one of the foregoing.

41. A pharmaceutical composition comprising the compound of any one of claims 1 to 40 and a pharmaceutically acceptable carrier.

42. A method of treating a subject having or suspected of having a cancer, comprising administering to the subject a therapeutically effective amount of the compound of any one of claims 1 to 40 or the pharmaceutical composition of claim 41.

43. The method of claim 42, wherein the cancer is a solid tumor or a hematological cancer.

44. The method of claim 42, wherein the cancer is a breast cancer, multiple myeloma, plasma cell myeloma, leukemia, lymphoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, chronic lymphocytic leukemia, prostate cancer, small cell lung cancer, or spleen cancer.

45. The method of any one of claims 42-44, wherein the compound is administered as monotherapy.

46. The method of any one of claims 42-44, wherein the compound is administered adjunctive to another therapeutic agent or radiation therapy.

47. The method of claim 46, wherein the compound is administered in an amount effective to sensitize the tumor cells to one or more additional therapeutic agents and/or radiation therapy.

48. The method of any one of claims 42-43, further comprising administering to the subject in need thereof at least one additional therapeutic agent.

49. The method of claim 48, wherein the additional therapeutic agent is a Bcl-2 inhibitor, a taxane, a MEK inhibitor, an ERK inhibitor, or a RAF inhibitor.
